(19) United States
(12) Patent Application Publication
(10) Pub. No.: US 2026/0217888 A1
(43) Pub. Date: Jul. 30, 2026

(54) SULFONIUM SALT MONOMER, POLYMER, CHEMICALLY AMPLIFIED RESIST COMPOSITION, AND PATTERN FORMING METHOD

(71) Applicant: Shin-Etsu Chemical Co., Ltd., Tokyo (JP)

(72) Inventor: Masahiro Fukushima, Joetsu-shi (JP)

(73) Assignee: Shin-Etsu Chemical Co., Ltd., Tokyo (JP)

(21) Appl. No.: 19/449,843

(22) Filed: Jan. 15, 2026

(30) Foreign Application Priority Data
Jan. 24, 2025 (JP) 2025-010463

(51) Int. Cl. C08F 220/22; C08F 212/14; C08F 220/18; C08F 220/30; G03F 7/00; G03F 7/004; G03F 7/039

(52) U.S. Cl. CPC C08F 220/22; C08F 212/24; C08F 220/1806; C08F 220/1807; C08F 220/301; G03F 7/0045; G03F 7/0397; G03F 7/70033

(57) ABSTRACT

The polymer contains a repeat unit derived from a sulfonium salt containing a carboxylic acid anion having a styrene or vinylnaphthalene structure as a polymerizable group and an iodine atom and a sulfonium cation having a pentafluorosulfanyl group and an acid-unstable group having a tertiary/secondary ether structure, a tertiary/secondary carbonate structure, or an acetal structure as a polymer-bound quencher. The sulfonium salt monomer having the following formula (a), wherein Z⁻ is a carboxylic acid anion having an aromatic vinyl structure and an iodine atom.

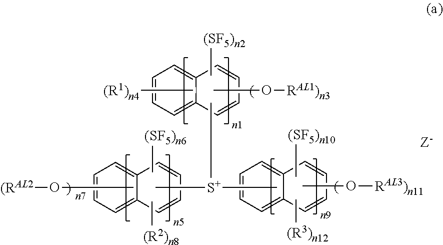

### CROSS-REFERENCE TO RELATED APPLICATION

This non-provisional application claims priority under 35 U.S.C. § 119(a) on Patent Application No. 2025-010463 filed in Japan on Jan. 24, 2025, the entire contents of which are hereby incorporated by reference.

### TECHNICAL FIELD

The present invention relates to a sulfonium salt monomer, a polymer, a chemically amplified resist composition, and a pattern forming method.

### BACKGROUND ART

With high integration and high speed of LSI, miniaturization of a pattern rule is rapidly progressing. In particular, expansion of a flash memory market and an increase in storage capacity lead to miniaturization. As the most advanced miniaturization technique, mass production of a 65 nm node device by ArF lithography is performed, and preparation for mass production of a 45 nm node device by next-generation ArF immersion lithography is in progress. For a next-generation 32 nm node device, immersion lithography using an ultra-high NA lens in which a liquid having a refractive index higher than water, a high refractive index lens, and a high refractive index resist film are combined, extreme ultraviolet (EUV) lithography with a wavelength of 13.5 nm, double exposure (double patterning lithography) of ArF lithography, and the like are candidates, and studies are being conducted.

As miniaturization progresses and a diffraction limit of light is approached, contrast of light decreases. Due to a decrease in contrast of light, resolution of a hole pattern or a trench pattern, and a focus margin decrease in a positive resist film.

Line width roughness (LWR) of a line pattern and dimensional uniformity (CDU) of a hole pattern have been regarded as problems along with miniaturization of a pattern. An influence of uneven distribution and aggregation of a base polymer and an acid generator and an influence of acid diffusion have been pointed out. Furthermore, LWR tends to increase as a resist film is thinned, and deterioration of LWR due to thinning with progress of miniaturization has become a serious problem.

In a resist composition for EUV lithography, it is necessary to simultaneously achieve high sensitivity, high resolution, and low LWR. If an acid diffusion distance is shortened, LWR is decreased but sensitivity is lowered. For example, by lowering a post exposure bake (PEB) temperature, LWR is decreased but sensitivity is lowered. By increasing the addition amount of a quencher, LWR is decreased but sensitivity is lowered. It is necessary to break a trade-off relationship between sensitivity and LWR.

In order to suppress acid diffusion, a resist compound containing a repeat unit derived from a sulfonic acid onium salt having a polymerizable unsaturated bond has been proposed (Patent Document 1). Such a so-called polymer-bound acid generator is characterized by very short acid diffusion because a polymeric sulfonic acid is generated by exposure. In addition, sensitivity can also be improved by increasing a ratio of the acid generator. If the addition amount of an addition-type acid generator is increased, sensitivity is increased, but in this case, an acid diffusion distance is also increased. Since an acid diffuses non-uniformly, LWR and CDU deteriorate as acid diffusion increases. It can be said that the polymeric acid generator has high ability in a balance among sensitivity, LWR, and CDU.

Since an iodine atom has very large absorption of EUV having a wavelength of 13.5 nm, an effect of generating secondary electrons from the iodine atom during exposure has been confirmed, and the iodine atom has attracted attention in EUV lithography. Patent Document 2 describes a photoacid generator in which an iodine atom is introduced into an anion, and Patent Document 3 describes a polymerizable group-containing photoacid generator in which an iodine atom is introduced into an anion. As a result, although improvement in lithographic performance to some extent has been confirmed, an iodine atom does not have high organic solvent solubility, and there is a concern about deposition thereof in a solvent.

In order to further suppress acid diffusion, a resist composition using a polymer containing a repeat unit derived from a sulfonium salt of a weak acid having a pKa of −0.8 or more, the sulfonium salt having a polymerizable group, as a polymer-bound quencher has been proposed (Patent Documents 4 to 8). In Patent Document 4, examples of the weak acid include carboxylic acid, sulfonamide, phenol, and hexafluoroalcohol.

### CITATION LIST

Patent Document 1: JP 4425776
Patent Document 2: JP 6720926
Patent Document 3: JP 6973274
Patent Document 4: WO 2019/167737
Patent Document 5: WO 2022/264845
Patent Document 6: JP-A 2022-115072
Patent Document 7: JP 7433394
Patent Document 8: JP-A 2024-178693

### SUMMARY OF THE INVENTION

In a chemically amplified resist composition using an acid as a catalyst, it is desired to develop a resist composition which has higher sensitivity, can improve lithographic performance such as LWR, CDU, exposure latitude (EL), or depth of focus (DOF), and is excellent in etching resistance after pattern formation.

The present invention has been made in view of the above circumstances, and an object thereof is to provide: a sulfonium salt monomer used in a chemically amplified resist composition which is excellent in solvent solubility, has high sensitivity and high contrast, is excellent in lithographic performance such as LWR, CDU, EL, or DOF, is resistant to pattern collapse even in fine pattern formation, and is excellent in etching resistance in photolithography using a high energy ray such as KrF excimer laser, ArF excimer laser, an electron beam (EB), or EUV; a polymer containing a repeat unit derived from the sulfonium salt monomer; a chemically amplified resist composition containing the polymer; and a pattern forming method using the chemically amplified resist composition.

As a result of intensive studies to achieve the above object, the present inventor has found that by using a polymer containing a repeat unit derived from a sulfonium salt containing a carboxylic acid anion having a styrene or vinylnaphthalene structure as a polymerizable group and an iodine atom and a sulfonium cation having a pentafluorosulfanyl group and an acid-unstable group having a tertiary/secondary ether structure, a tertiary/secondary carbonate structure, or an acetal structure as a polymer-bound quencher, a chemically amplified resist composition which is excellent in solvent solubility, has high sensitivity, has improved lithographic performance such as LWR, CDU, EL, or DOF, has high contrast, has high resolution, and is excellent in etching resistance can be obtained, and has completed the present invention.

That is, the present invention provides the following sulfonium salt monomer, polymer, chemically amplified resist composition, and pattern forming method.
1. A sulfonium salt monomer having the following formula (a):

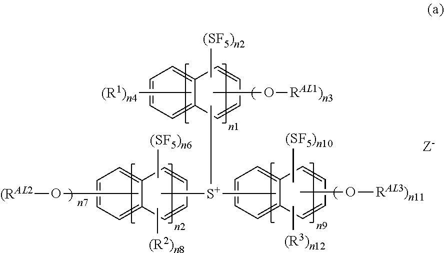

wherein n1 is 0 or 1, n2 is 0, 1, or 2, n3 is 0, 1, or 2, n4 is 0, 1, or 2, provided that 0≤n2+n3+n4≤5 when n1 is 0, and 0≤n2+n3+n4≤7 when n1 is 1, n5 is 0 or 1, n6 is 0, 1, or 2, n7 is 0, 1, or 2, n8 is 0, 1, or 2, provided that 0≤n6+n7+n8≤5 when n5 is 0, and 0≤n6+n7+n8≤7 when n5 is 1, n9 is 0 or 1, n10 is 0, 1, or 2, nll is 0, 1, or 2, n12 is 0, 1, or 2, provided that 0≤n10+nl1+n12≤5 when n9 is 0, and 0≤n10+n11+n12≤7 when n9 is 1, 1≤n2+n6+n10≤6, 1≤n3+n7+n11≤6,
   R¹, R², and R³ are each independently a halogen atom, a nitro group, a cyano group, a hydroxy group, a carboxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n4 is 2, R¹s may be the same as or different from each other, and two R¹s may be bonded to each other to form a ring together with carbon atoms to which they are bonded, when n8 is 2, R²s may be the same as or different from each other, and two R²s may be bonded to each other to form a ring together with carbon atoms to which they are bonded, when n12 is 2, R³s may be the same as or different from each other, and two R³s may be bonded to each other to form a ring together with carbon atoms to which they are bonded,
   R^{AL1}, R^{AL2}, and R^{AL3} are each independently an acid-unstable group, when n3 is 2, R^{AL1}s may be the same as or different from each other, when n7 is 2, R^{AL2}s may be the same as or different from each other, when n11 is 2, R^{AL3}s may be the same as or different from each other,
   at least one —SF₅ group is bonded to a carbon atom adjacent to a carbon atom to which —O—R^{AL1}, —O—R^{AL2}, or —O—R^{AL3} is bonded,
   two of the three aromatic rings bonded to S⁺ may be bonded to each other to form a ring together with the sulfur atom to which they are bonded, and
   Z⁻ is a carboxylic acid anion having an aromatic vinyl structure and an iodine atom.
2. The sulfonium salt monomer according to the item 1, having the following formula (al):

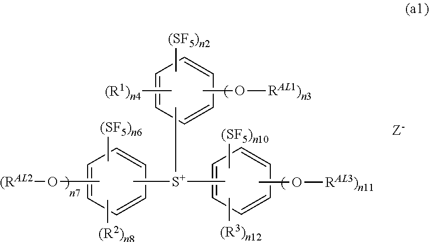

wherein n2 to n4, n6 to n8, n10 to n12, R^{AL1} to R^{AL3}, R¹ to R³, and Z⁻ are as defined above.
3. The sulfonium salt monomer according to the item 1 or 2, wherein the acid-unstable group has the following formula (AL-1) or (AL-2):

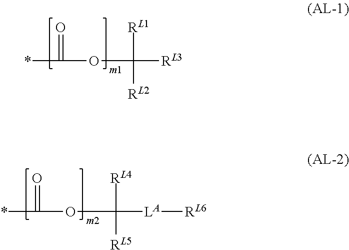

wherein m1 and m2 are each independently 0 or 1,
   R^{L1} and R^{L2} are each independently a C₁-C₁₂ hydrocarbyl group, R^{L3} is a hydrogen atom or a C₁-C₁₂ hydrocarbyl group, some of (—CH2-)s of the hydrocarbyl group of R^{L1}, R^{L2}, and R^{L3} may be replaced with —O— or —S—, when the hydrocarbyl group contains an aromatic ring, some or all of hydrogen atoms of the aromatic ring may be replaced with a halogen atom, a cyano group, a nitro group, a C₁-C₄ alkyl group which may contain a halogen atom, or a C₁-C₄ alkoxy group which may contain a halogen atom, R^{L1} and R^{L2} may be bonded to each other to form a ring together with a carbon atom to which they are bonded, and some of (—CH₂-)s of the ring may be replaced with —O— or —S—, provided that when R^{L3} is a hydrogen atom, R^{L1} and R^{L2} are bonded to each other to form an alicyclic ring containing a multiple bond together with a carbon atom to which they are bonded,
   R^{L4} and R^{L5} are each independently a hydrogen atom or a C₁-C₁₀ hydrocarbyl group, R^{L6} is a C₁-C₂₀ hydrocarbyl group, and some of (—CH₂—)s of the hydrocarbyl group may be replaced with —O— or —S—, R^{L5} and R^{L6} may be bonded to each other to form a C₃-C₂₀ heterocyclic group together with a carbon atom and L^{A} to which they are bonded, and some of (—CH₂—)s of the heterocyclic group may be replaced with —O— or —S—,
   L^{A} is —O— or —S—, and
   * represents a bond with adjacent —O—.
4. The sulfonium salt monomer according to any one of the items 1 to 3, wherein Z⁻ is an anion having the following formula (Z):

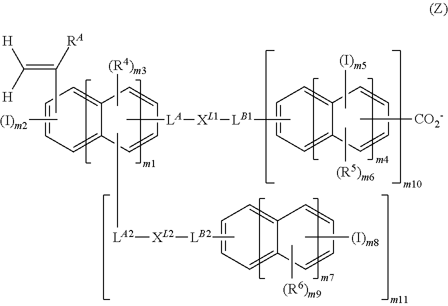

wherein m1 is 0 or 1, m2 is 0, 1, 2, 3, or 4, m3 is 0, 1, 2, or 3, m4 is 0 or 1, m5 is 0, 1, 2, 3, or 4, m6 is 0, 1, 2, or 3, m7 is 0 or 1, m8 is 1, 2, 3, or 4, m9 is 0, 1, 2, or 3, m10 is 0 or 1, m11 is 0 or 1, provided that 0≤m2+m3+m11≤4 when m1 is 0, and 0≤m2+m3+m11≤6 when m1 is 1, 0≤m5+m6≤4 when m4 is 0, and 0≤m5+m6≤6 when m4 is 1, 0≤m8+m9≤5 when m7 is 0, and 0≤m8+m9≤7 when m7 is 1, 1≤m2+m5+m8≤4,
   R^{A} is a hydrogen atom, a fluorine atom, a methyl group, or a trifluoromethyl group,
   R⁴, R⁵, and R⁶ are each independently a halogen atom other than an iodine atom, a nitro group, a cyano group, a hydroxy group, a carboxy group, a pentafluorosulfanyl group, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₁-C₂₀ hydrocarbyloxycarbonyl group which may contain a heteroatom, when m3 is 2 or 3, R⁴s may be the same as or different from each other, and two R⁴s may be bonded to each other to form a ring together with carbon atoms to which they are bonded, when m6 is 2 or 3, R⁵s may be the same as or different from each other, and two R⁵s may be bonded to each other to form a ring together with carbon atoms to which they are bonded, when m9 is 2 or 3, R⁶s may be the same as or different from each other, and two R⁶s may be bonded to each other to form a ring together with carbon atoms to which they are bonded,
   L^{A1}, L^{A2}, L^{B1}, and L^{B2} are each independently a single bond, an ether bond, a carbonyl group, an ester bond, a sulfonate bond, an amide bond, a sulfonamide bond, a carbonate bond, or a carbamate bond, and
   X^{L1} and X^{L2} are each independently a single bond or a C₁-C₄₀ hydrocarbylene group which may contain a heteroatom.
5. A sulfonium salt quencher containing the sulfonium salt monomer according to any one of the items 1 to 4.
6. A polymer containing a repeat unit derived from the sulfonium salt quencher according to the item 5.
7. The polymer according to the item 6, further containing at least one selected from a repeat unit having the following formula (b1), a repeat unit having the following formula (b2), and a repeat unit having the following formula (b3):

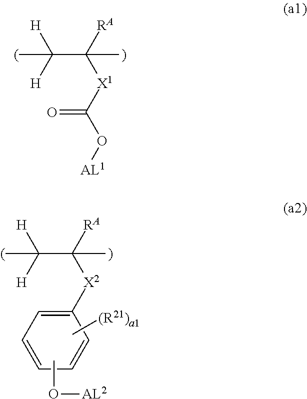

wherein R^{A}s are each independently a hydrogen atom, a fluorine atom, a methyl group, or a trifluoromethyl group,
   X¹ is a single bond, a phenylene group, a naphthylene group, *—C(═O)—O—X¹¹—, or *—C(═O)—N(H)—X¹¹—, and the phenylene group or the naphthylene group may be substituted with a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom, X¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring,
   X² is a single bond, *—C(═O)—O—, or *—C(—O)—N(H)—,
   * represents a bond with a carbon atom of a main chain,
   R¹¹ is a halogen atom, a cyano group, a hydroxy group, a nitro group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, when al is 2, 3, or 4, R¹¹s may be the same as or different from each other,
   AL¹ and AL² are each independently an acid-unstable group, and
   a1 is 0, 1, 2, 3, or 4.

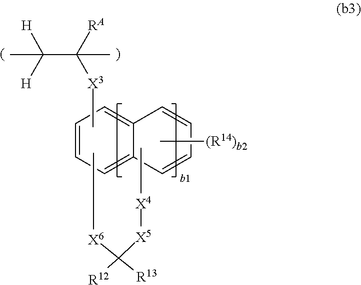

wherein b1 is 0 or 1, when b1 is 0, b2 is 0, 1, 2, or 3, and when b1 is 1, b2 is 0, 1, 2, 3, 4, or 5,
   R^{A} is a hydrogen atom, a fluorine atom, a methyl group, or a trifluoromethyl group,
   X³ is a single bond, *—C(═O)—O—, or *—C(═O)—N(H)—, * represents a bond with a carbon atom of a main chain,
   X⁴ is a single bond, a C₁-C₄ aliphatic hydrocarbylene group, a carbonyl group, a sulfonyl group, or a group obtained by combining these groups,
   X⁵ and X⁶ are each independently an oxygen atom or a sulfur atom, provided that X⁴ and X⁶ are bonded to adjacent aromatic ring carbon atoms.
   R¹² and R¹³ are each independently a hydrogen atom or a C₁-C₂₀ hydrocarbyl group which may contain a heteroatom, R¹² and R¹³ may be bonded to each other to form a ring together with a carbon atom to which they are bonded,
   R¹⁴ is a halogen atom, a hydroxy group, a cyano group, a nitro group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A})(R^{14B}), R^{14A} and R^{14B} are each independently a hydrogen atom or a C₁-C₆ hydrocarbyl group, when b2 is 2 or more, R¹⁴s may be the same as or different from each other, and a plurality of R¹⁴s may be bonded to each other to form a ring together with carbon atoms to which they are bonded.
8. The polymer according to the item 6 or 7, further containing a repeat unit having the following formula (c):

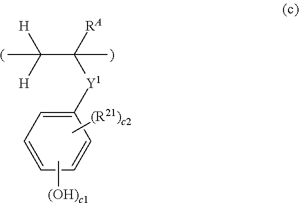

wherein R^{A} is a hydrogen atom, a fluorine atom, a methyl group, or a trifluoromethyl group,
   Y¹ is a single bond, *—C(═O)—O—, or *—C(═O)—N(H)—, * represents a bond with a carbon atom of a main chain,
   R²¹ is a halogen atom, a carboxy group, a nitro group, a cyano group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom,
   c1 is 1, 2, 3, or 4, and c2 is 0, 1, 2, or 3, provided that 1≤c1+c2≤5.
9. The polymer according to any of the items 6 to 8, further containing a repeat unit derived from an onium salt monomer containing a fluorosulfonic acid anion having a polymerizable group and at least one iodine atom and a sulfonium cation.
10. The polymer according to any one of the items 6 to 9, further containing a repeat unit having the following formula (e):

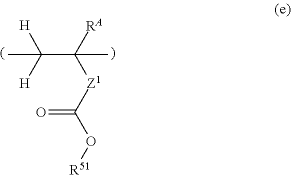

wherein R^{A} is a hydrogen atom, a fluorine atom, a methyl group, or a trifluoromethyl group,
   Z¹ is a single bond, a phenylene group, a naphthylene group, *—C(═O)—O—Z¹¹—, or *—C(═O)—N(H)—Z¹¹—, and the phenylene group or the naphthylene group may have a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom as a substituent, * represents a bond with a carbon atom of a main chain, Z¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring,
   R⁵¹ is a hydrogen atom or a C₁-C₂₀ group containing at least one structure selected from a hydroxy group other than a phenolic hydroxy group, a cyano group, a carbonyl group, a carboxy group, an ether bond, an ester bond, a sulfonate bond, a carbonate bond, a lactone ring, a sultone ring, and a carboxylic anhydride (—C(═O)—O—C(═O)—),
11. A chemically amplified resist composition containing (A) a base polymer containing the polymer according to any one of the items 6 to 10.
12. The chemically amplified resist composition according to the item 11, further containing (B) an organic solvent.
13. The chemically amplified resist composition according to the item 11 or 12, further containing (C) a quencher.
14. The chemically amplified resist composition according to any one of the items 11 to 13, further containing (D) an acid generator.
15. The chemically amplified resist composition according to any one of the items 11 to 14, further containing (E) a surfactant.
16. A pattern forming method including: a step of forming a resist film on a substrate using the chemically amplified resist composition according to any one of the items 11 to 15; a step of exposing the resist film to a high energy ray; and a step of developing the exposed resist film using a developer.
17. The pattern forming method according to the item 16, wherein the high energy ray is ArF excimer laser having a wavelength of 193 nm, KrF excimer laser having a wavelength of 248 nm, an electron beam, or an extreme ultraviolet ray having a wavelength of 3 to 15 nm.

### Advantageous Effects of the Invention

A resist film containing a polymer containing a repeat unit derived from the sulfonium salt monomer having the formula (a) has good solvent solubility, and has small acid diffusion because the atomic weight of an iodine atom is large. This makes it possible to prevent a decrease in resolution due to blurring of acid diffusion and to improve lithographic performance such as LWR, CDU, EL, or DOF. In addition, since absorption of EUV having a wavelength of 13.5 nm by an iodine atom is very large, secondary electrons are generated from the iodine atom during exposure, and sensitivity is increased. Furthermore, a pentafluorosulfanyl group contained in the cation is a strong electron-withdrawing group, and is bonded to an aromatic ring of the sulfonium cation, whereby LUMO in the frontier molecule activation theory is lowered, and the sulfonium cation easily receives secondary electrons, which promotes decomposition of the sulfonium cation. In addition, the acid-unstable group having a tertiary/secondary ether structure, a tertiary/secondary carbonate structure, or an acetal structure, bonded to an aromatic ring of the sulfonium cation, undergoes a deprotection reaction with an acid generated by exposure to generate a corresponding phenol. By change of the structure from a fat-soluble structure to a hydrophilic structure, contrast between an exposed portion and an unexposed portion is improved. In addition, a pentafluorosulfanyl group is bonded to a carbon atom adjacent to a carbon atom to which a phenolic hydroxy group to be generated is bonded, whereby acidity of the phenol is improved due to an electron withdrawing property of the pentafluorosulfanyl group. When an exposed portion in which the deprotection reaction has progressed is developed with an alkaline developer, the phenol having an improved acidity has improved solubility in the alkaline developer, whereby development residues can be suppressed, and collapse of a resist pattern can be suppressed by suppression of swelling by the alkaline developer. On the other hand, the carboxylic acid anion has relatively high basicity, and can effectively trap an acid generated from a strong acid. This makes it possible to construct a chemically amplified resist composition having improved lithographic performance such as LWR, CDU, EL, or DOF while having high sensitivity. In addition, the aromatic ring acts as a good etching-resistant group, and is suitable for fine pattern formation.

### DESCRIPTION OF THE PREFERRED EMBODIMENT

### [Sulfonium Salt Monomer]

A sulfonium salt monomer of the present invention has the following formula (a):

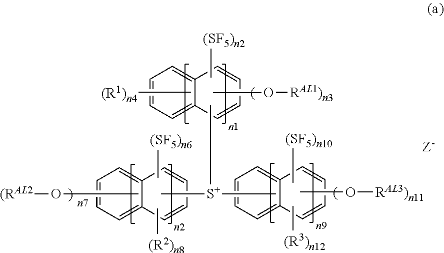

In the formula (a), n1 is 0 or 1. When n1 is 0, it is a benzene ring, and when n1 is 1, it is a naphthalene ring, but a benzene ring in which n1 is 0 is preferable from a viewpoint of solvent solubility. n2 is 0, 1, or 2. n3 is 0, 1, or 2. n4 is 0, 1, or 2. n4 is preferably 0 or 1 from a viewpoint of raw material procurement. Provided that 0≤n2+n3+n4≤5 when n1 is 0, and 0≤n2+n3+n4≤7 when n1 is 1.

In the formula (a), n5 is 0 or 1. When n5 is 0, it is a benzene ring, and when n5 is 1, it is a naphthalene ring, but a benzene ring in which n5 is 0 is preferable from a viewpoint of solvent solubility. n6 is 0, 1, or 2. n7 is 0, 1, or 2. n8 is 0, 1, or 2. n8 is preferably 0 or 1 from a viewpoint of raw material procurement. Provided that 0≤n6+n7+n8≤5 when n5 is 0, and 0≤n6+n7+n8≤7 when n5 is 1.

In the formula (a), n9 is 0 or 1. When n9 is 0, it is a benzene ring, and when n9 is 1, it is a naphthalene ring, but a benzene ring in which n9 is 0 is preferable from a viewpoint of solvent solubility. n10 is 0, 1, or 2. n11 is 0, 1, or 2. n12 is 0, 1, or 2. n12 is preferably 0 or 1 from a viewpoint of raw material procurement. Provided that 0≤n10+n11+n12≤5 when n9 is 0, and 0≤n10+n11+n12≤7 when n9 is 1.

In the formula (a), 1≤n2+n6+n10≤6, preferably 1≤n2+n6+n10≤3, and more preferably 1≤n2+n6+n10≤2. 1≤n3+n7+n11≤6, preferably 1≤n3+n7+n11≤3, and more preferably 1≤n3+n7+n11≤2.

In the formula (a), R¹, R², and R³ are each independently a halogen atom, a nitro group, a cyano group, a hydroxy group, a carboxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. Examples of the halogen atom include a fluorine atom, a chlorine atom, a bromine atom, and an iodine atom. The hydrocarbyl group and hydrocarbyl moieties of the hydrocarbyloxy group and the hydrocarbylthio group may be saturated or unsaturated, and may be linear, branched, or cyclic. Specific examples thereof include: a C₁-C₂₀ alkyl group such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, or an icosyl group; a C₃-C₂₀ cyclic saturated hydrocarbyl group such as a cyclopropyl group, a cyclopentyl group, a cyclohexyl group, a cyclopropylmethyl group, a 4-methylcyclohexyl group, a cyclohexylmethyl group, a norbornyl group, or an adamantyl group; a C₂-C₂₀ alkenyl group such as a vinyl group, a 1-propenyl group, a 2-propenyl group, a butenyl group, or a hexenyl group; a C₃-C₂₀ cyclic unsaturated hydrocarbyl group such as a cyclohexenyl group; a C₆-C₂₀ aryl group such as a phenyl group or a naphthyl group; a C₇-C₂₀ aralkyl group such as a benzyl group, a 1-phenylethyl group, or a 2-phenylethyl group; and a group obtained by combining these groups. Among these, an aryl group is preferable. Some or all of hydrogen atoms of the hydrocarbyl group may be replaced with a group containing a heteroatom such as an oxygen atom, a sulfur atom, a nitrogen atom, or a halogen atom, some of (—CH₂—)s in the hydrocarbyl group may be replaced with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic acid anhydride (—C(═O)—O—C(—O)—), a haloalkyl group, or the like.

When n4 is 2, R¹s may be the same as or different from each other. When n4 is 2, two R¹s may be bonded to each other to form a ring together with carbon atoms to which they are bonded. The ring is preferably a 5-to 8-membered ring. When n8 is 2, R²s may be the same as or different from each other. When n8 is 2, two R²s may be bonded to each other to form a ring together with carbon atoms to which they are bonded. The ring is preferably a 5- to 8-membered ring. When n12 is 2, R³s may be the same as or different from each other. When n12 is 2, two R³s may be bonded to each other to form a ring together with carbon atoms to which they are bonded. The ring is preferably a 5- to 8-membered ring.

In the formula (a), R^{AL1}, R^{AL2}, and R^{AL3} are each independently an acid-unstable group. When n3 is 2, R^{AL1}s may be the same as or different from each other. When n7 is 2, R^{AL2}s may be the same as or different from each other. When n11 is 2, R^{AL3}s may be the same as or different from each other.

The acid-unstable group preferably has the following formula (AL-1) or (AL-2):

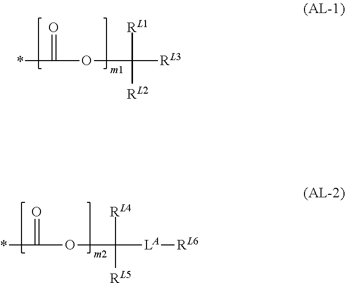

wherein * represents a bond.

In the formulas (AL-1) and (AL-2), m1 and m2 are each independently 0 or 1.

In the formula (AL-1), R^{L1} and R^{L2} are each independently a C₁-C₁₂ hydrocarbyl group. R^{L3} is a hydrogen atom or a C₁-C₁₂ hydrocarbyl group, some of (—CH₂—)s of the hydrocarbyl group of R^{L1}, R^{L2}, and R^{L3} may be replaced with —O— or —S—, when the hydrocarbyl group contains an aromatic ring, some or all of hydrogen atoms of the aromatic ring may be replaced with a halogen atom, a cyano group, a nitro group, a C₁-C₄ alkyl group which may contain a halogen atom, or a C₁-C₄ alkoxy group which may contain a halogen atom, The C₁-C₁₂ hydrocarbyl group of R^{L1}, R^{L2}, and R^{L3} may be saturated or unsaturated, and may be linear, branched, or cyclic. Specific examples thereof include those having 1 to 12 carbon atoms among those exemplified as the hydrocarbyl group of R¹, R², and R³. R^{L1} and R^{L2} may be bonded to each other to form a ring together with a carbon atom to which they are bonded, and some of (—CH₂—)s of the ring may be replaced with —O— or —S—. Provided that when R^{L3} is a hydrogen atom, R^{L1} and R^{L2} are bonded to each other to form an alicyclic ring containing a multiple bond together with a carbon atom to which they are bonded.

In the formula (AL-2), R^{L4} and R^{L5} are each independently a hydrogen atom or a C₁-C₁₀ hydrocarbyl group. The C₁-C₁₀ hydrocarbyl group of R^{L4} and R^{L5} may be saturated or unsaturated, and may be linear, branched, or cyclic. Specific examples thereof include those similar to those having 1 to 10 carbon atoms among those exemplified as the hydrocarbyl group of R¹, R², and R³. R^{L6} is a C₁-C₂₀ hydrocarbyl group, and some of (—CH₂—)s of the hydrocarbyl group may be replaced with —O— or —S—. R^{L5} and R^{L6} may be bonded to each other to form a C₃-C₂₀ heterocyclic group together with a carbon atom and L^{A} to which they are bonded, and some of (—CH₂—)s of the heterocyclic group may be replaced with —O— or —S—. The C₁-C₂₀ hydrocarbyl group of R^{L6} may be saturated or unsaturated, and may be linear, branched, or cyclic. Specific examples thereof include those similar to those exemplified as the hydrocarbyl group of R¹, R², and R³.

In the formula (AL-2), L^{A} is —O— or —S—.

Specific examples of the acid-unstable group having the formula (AL-1) include, but are not limited to, those listed below. Note that * represents a bond with adjacent —O— in the following formulas.

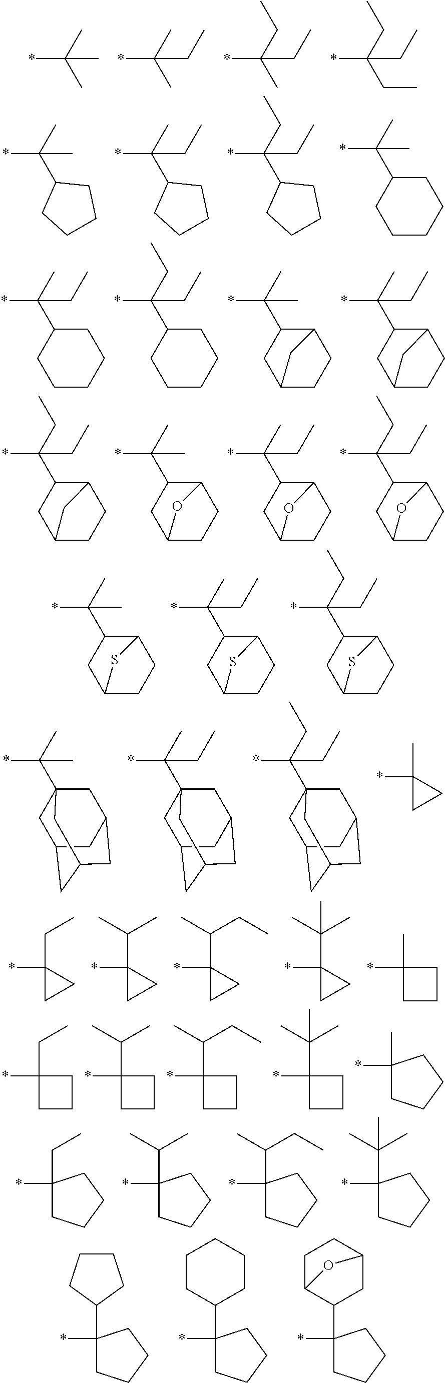

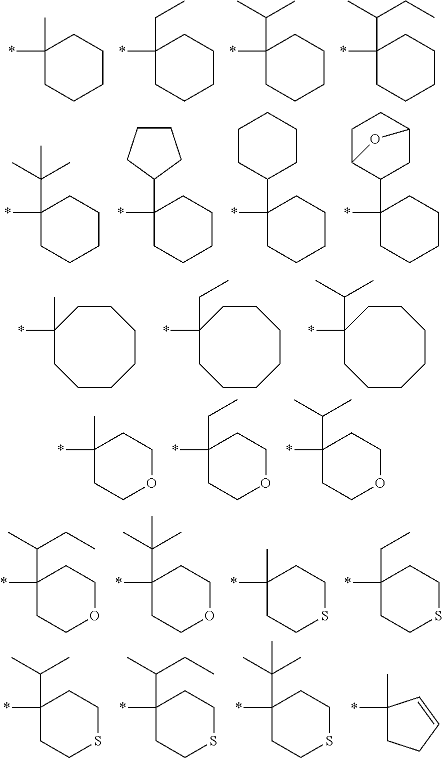

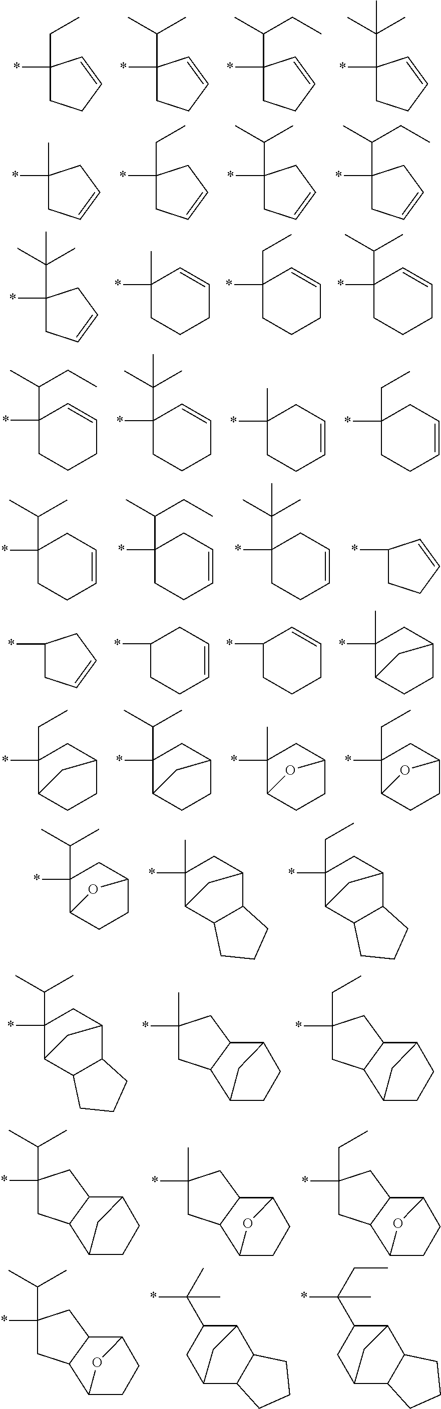

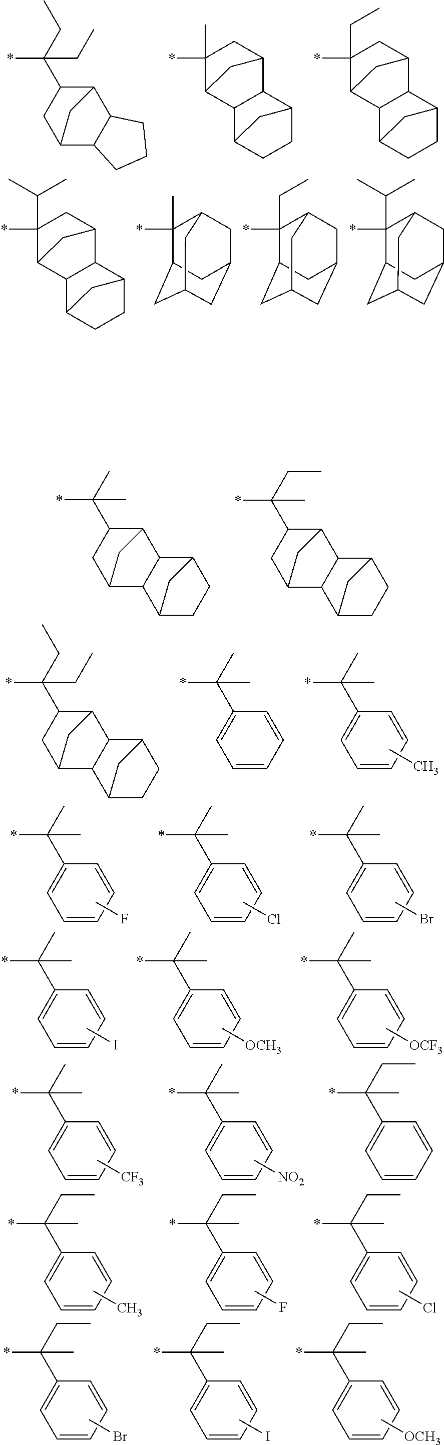

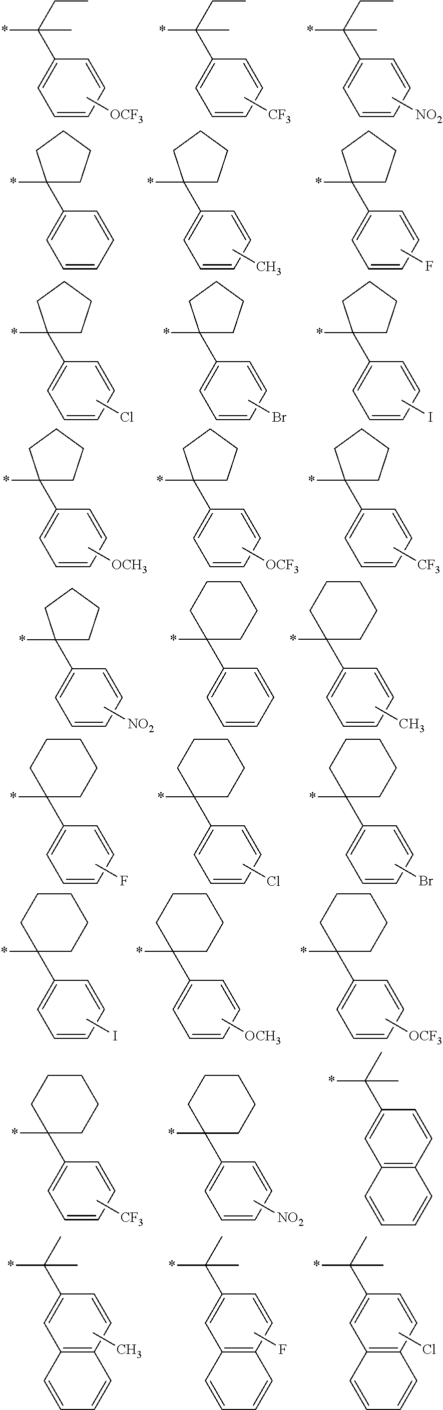

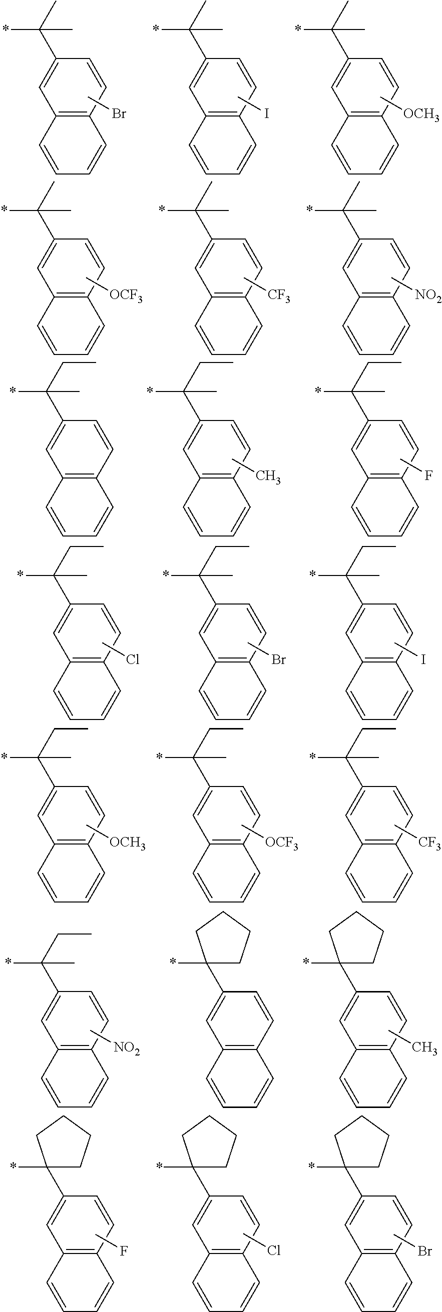

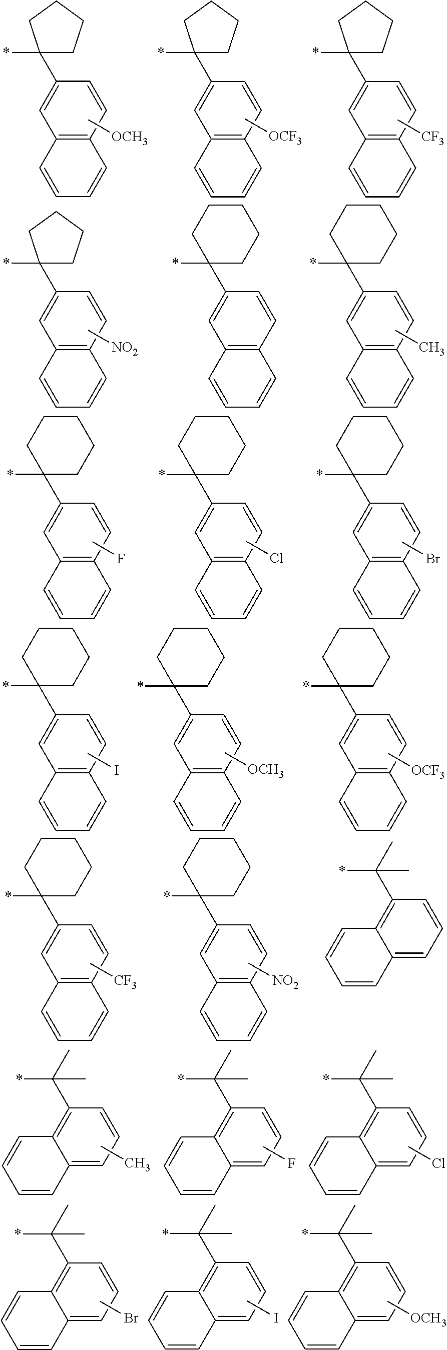

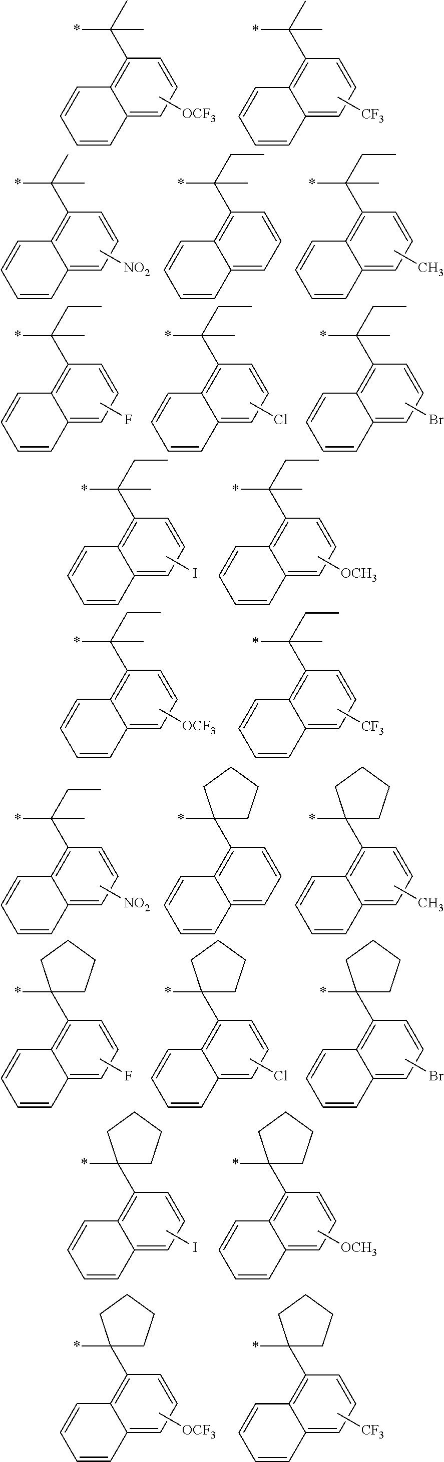

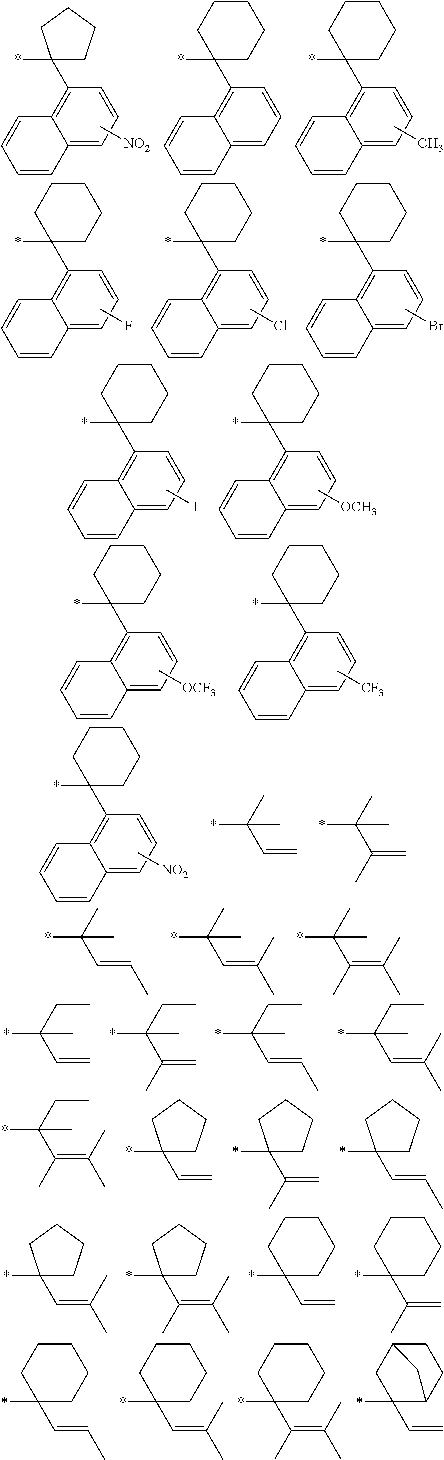

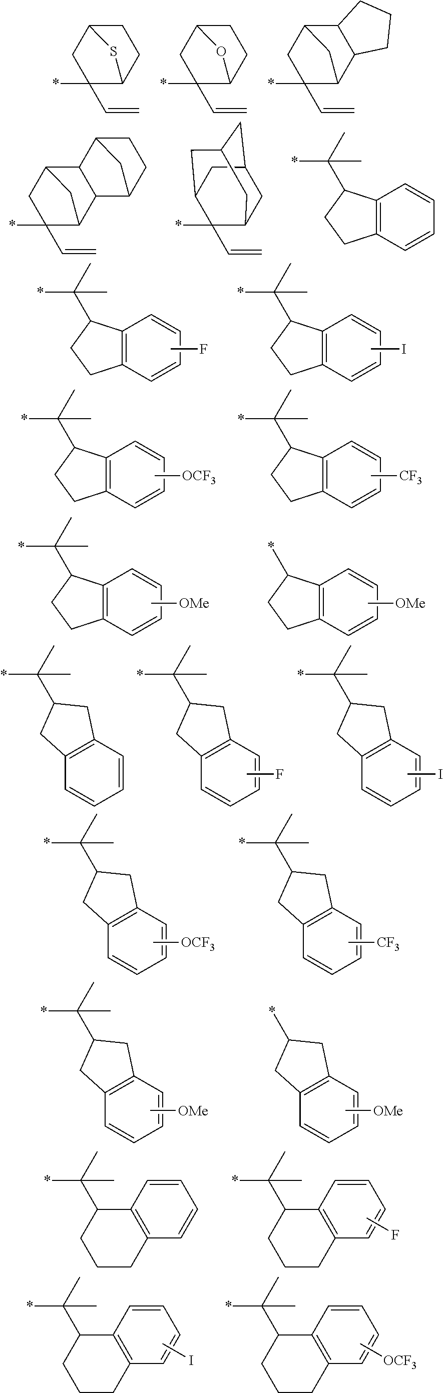

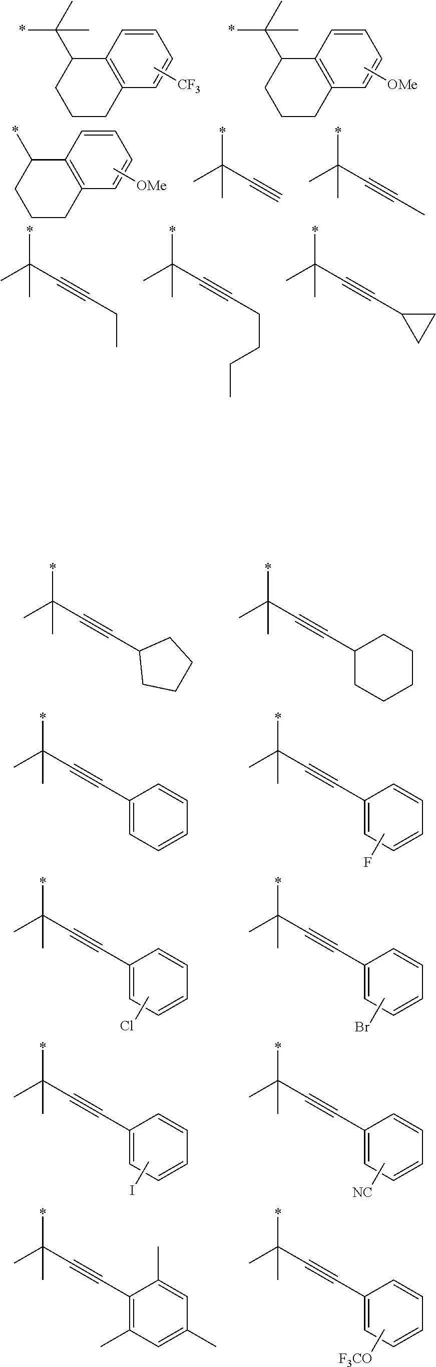

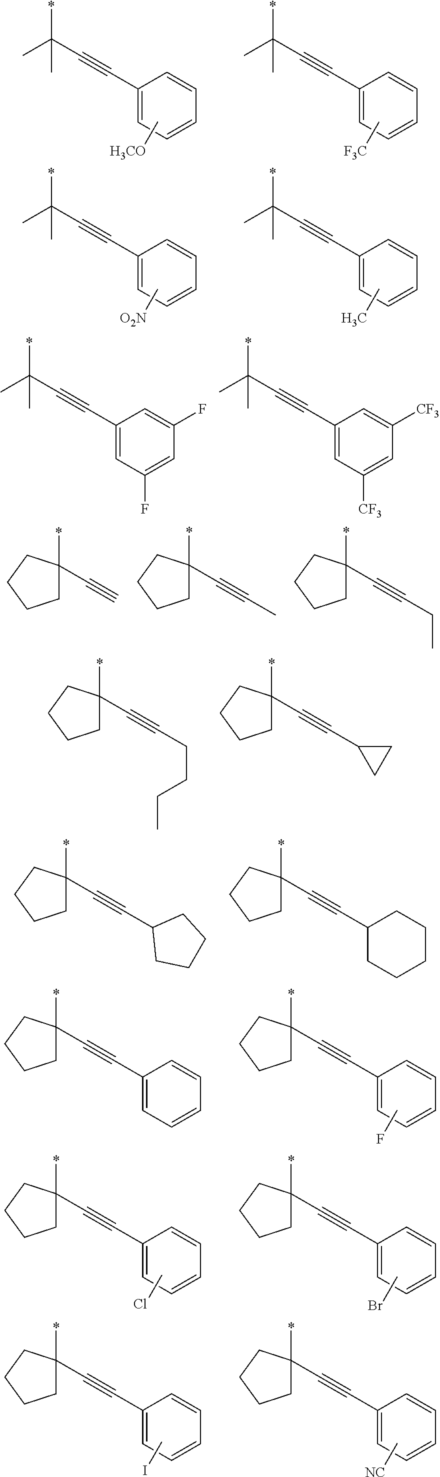

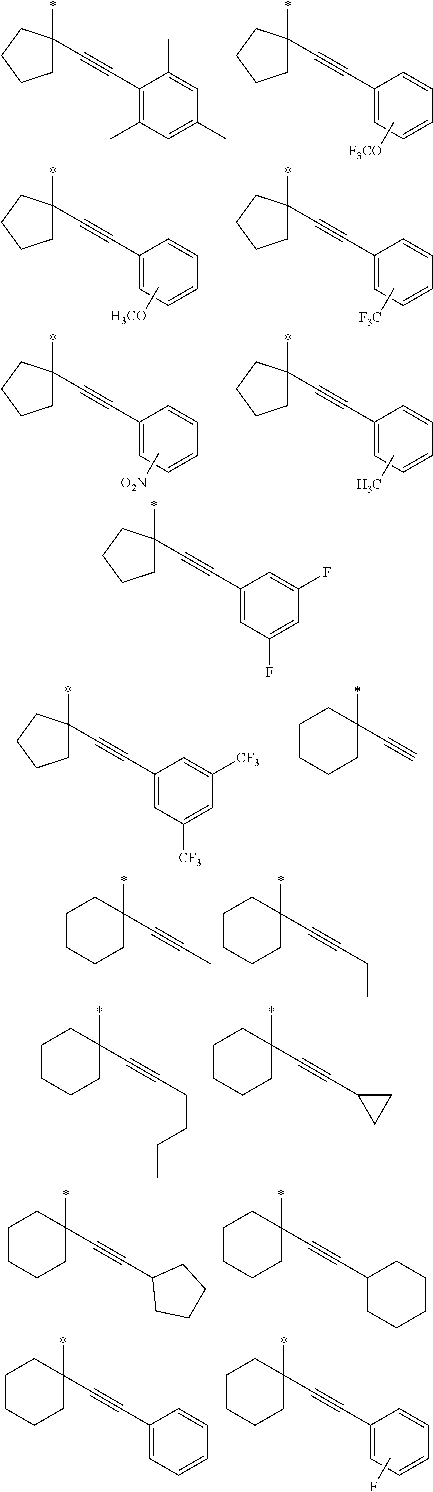

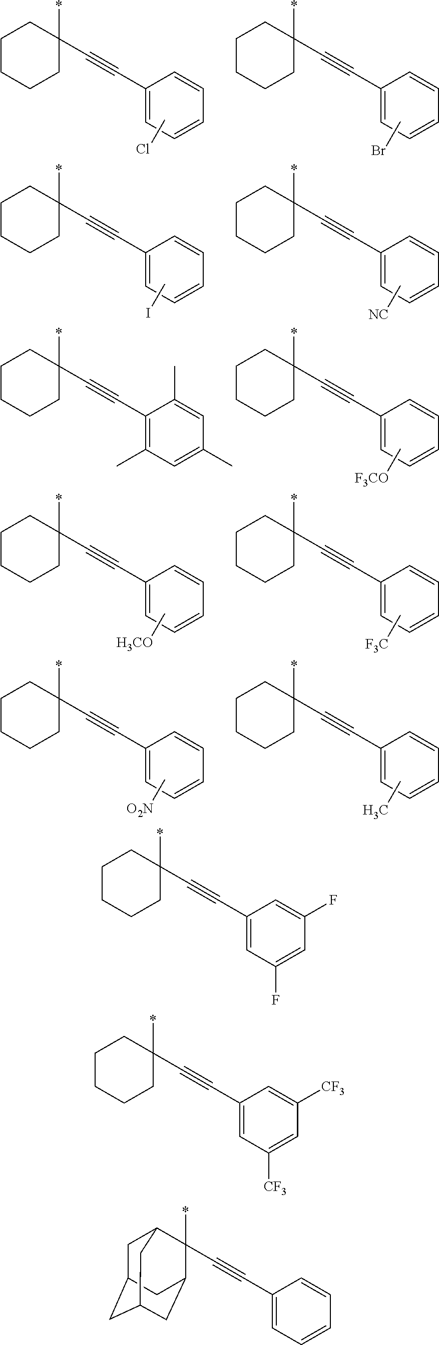

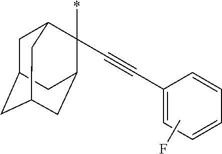

Specific examples of the acid-unstable group having the formula (AL-2) include, but are not limited to, those listed below. Note that * represents a bond with adjacent —O— in the following formulas.

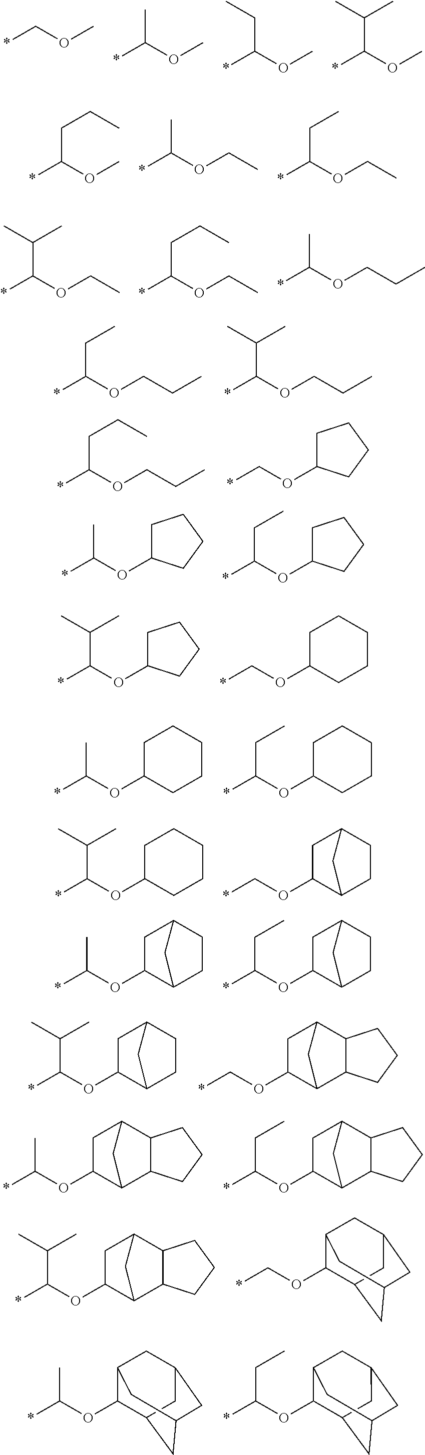

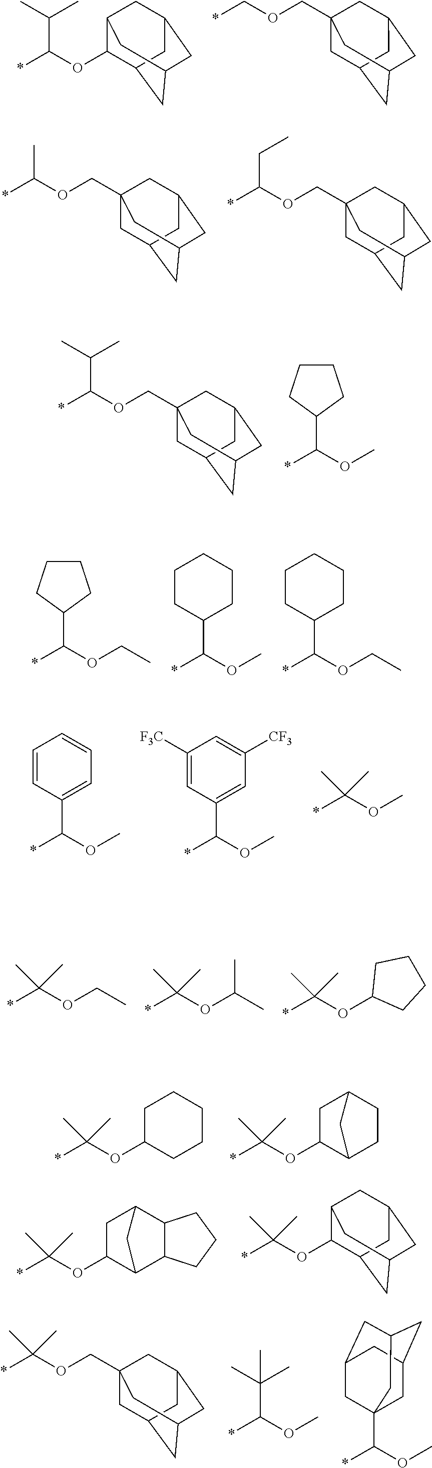

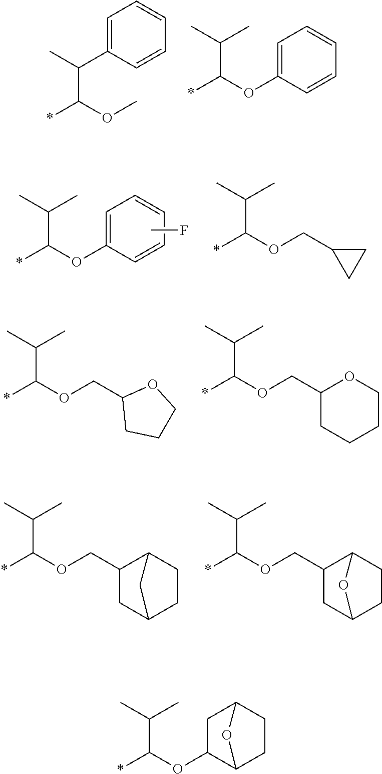

In the formula (a), at least one —SF₅ group is bonded to a carbon atom adjacent to a carbon atom to which —O—R^{AL1}, —O—R^{AL2}, or —O—R^{AL3} is bonded. The —SF₅ group and —O—R^{AL1}, —O—R^{AL2}, or —O—R^{AL3} are bonded to carbon atoms adjacent to each other, whereby acidity of a phenol after —R^{AL1}, —R^{AL2}, or —R^{AL3} is deprotected is improved, and dissolution contrast is improved.

Two of the three aromatic rings bonded to S⁺ may be bonded to each other to form a ring together with the sulfur atom to which they are bonded. At this time, specific examples of the structure of the ring include those having the following formulas:

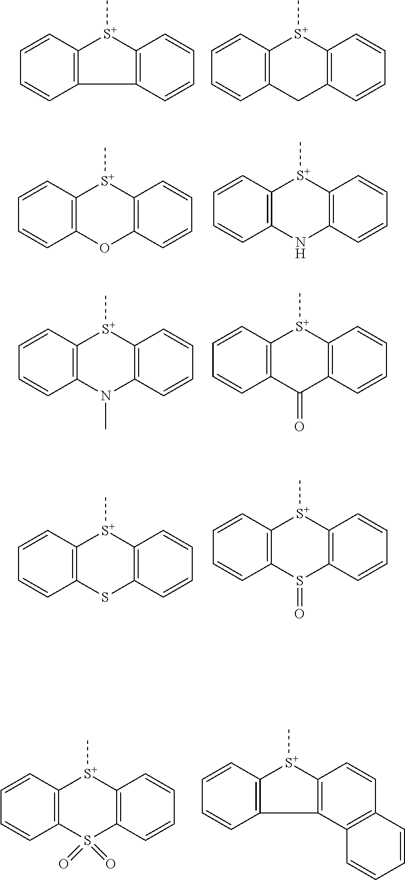

wherein the dashed line is a bond.

The sulfonium salt having the formula (a) preferably has the following formula (a1):

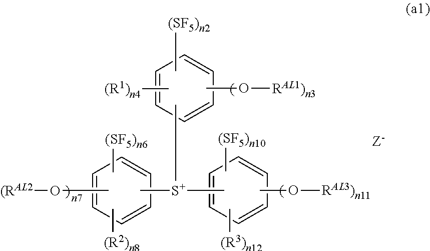

wherein n2 to n4, n6 to n8, n10 to n12, R^{AL1} to R^{AL3}, and R¹ to R³ are as defined above. Z⁻ is described below.

Specific examples of the cation of the sulfonium salt having the formula (a) include, but are not limited to, those listed below. Note that, in the following formulas, Me is a methyl group.

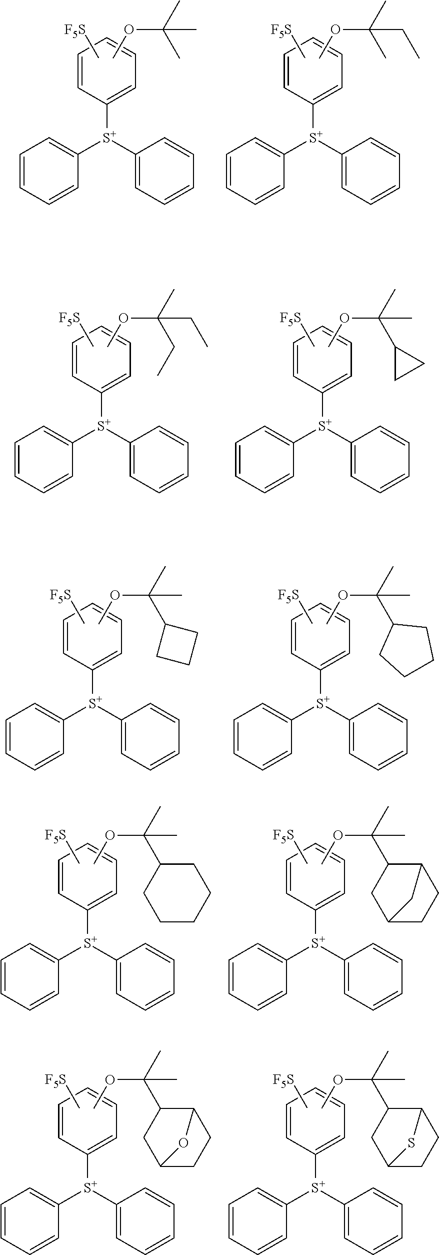

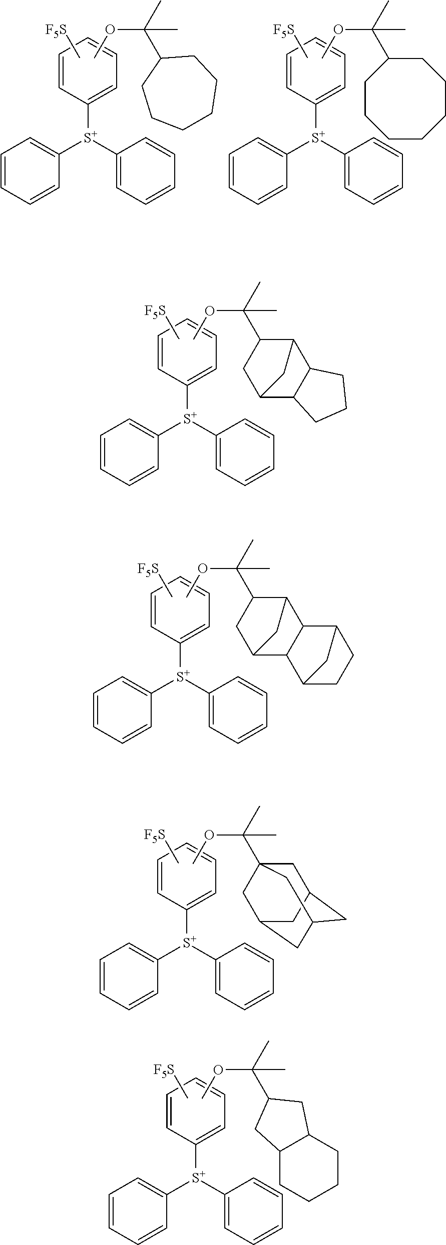

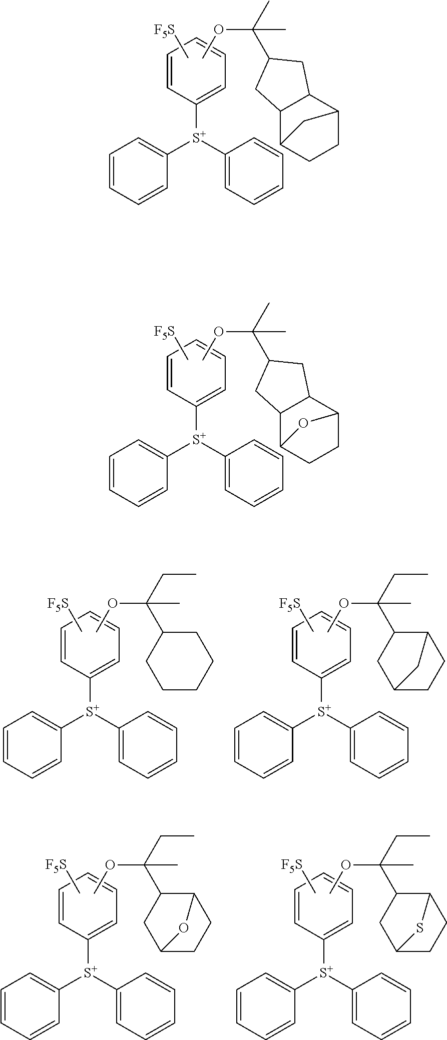

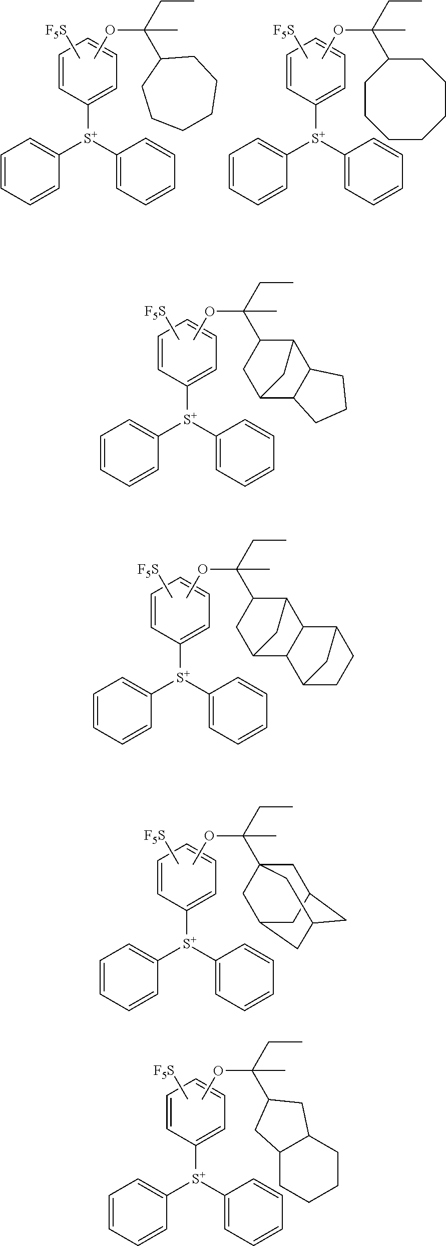

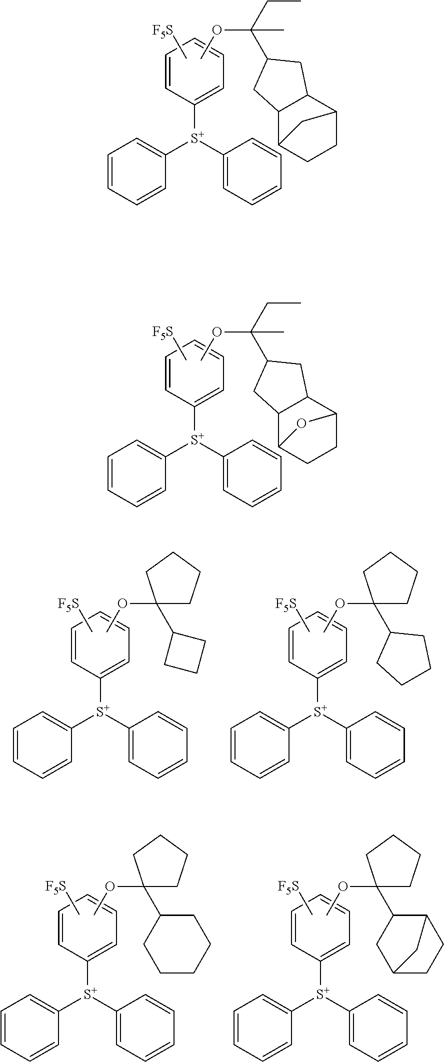

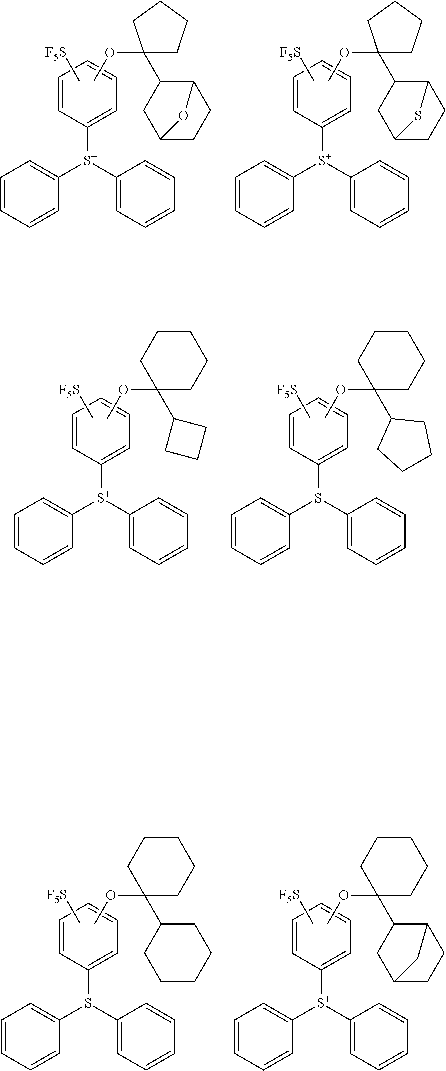

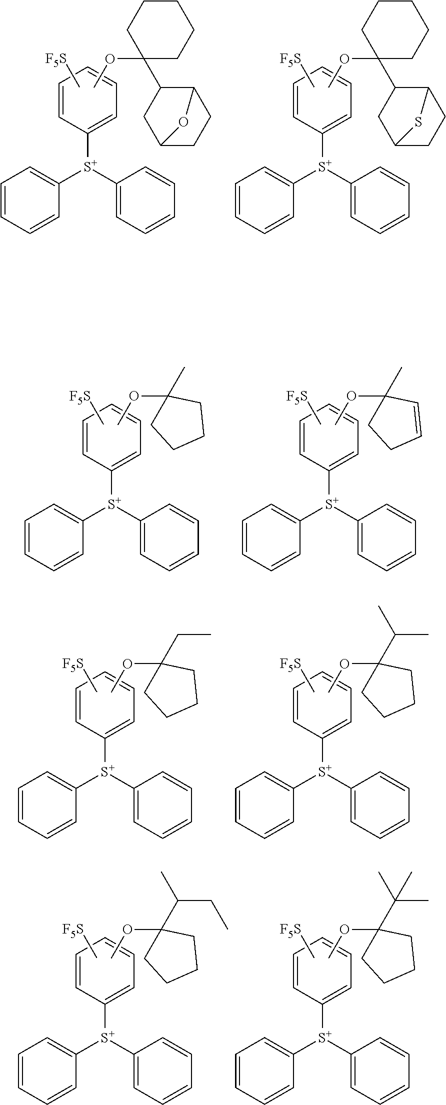

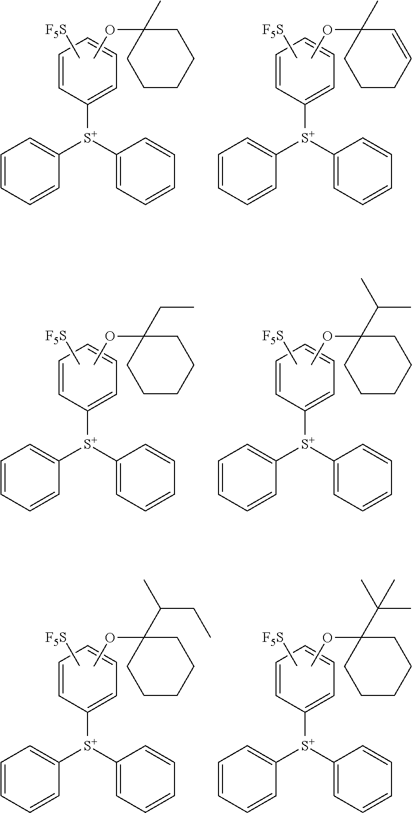

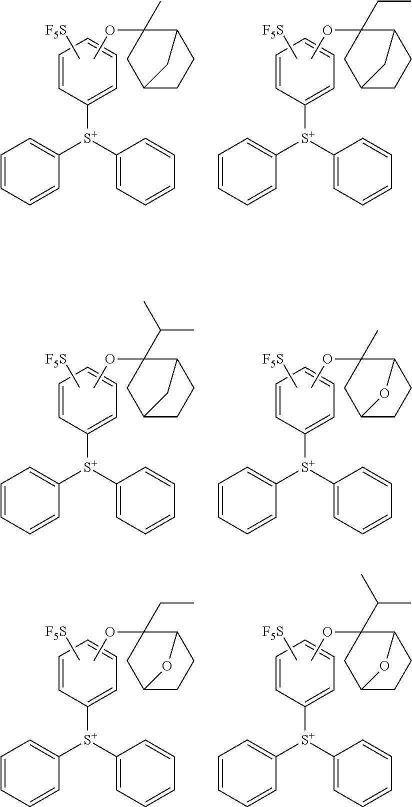

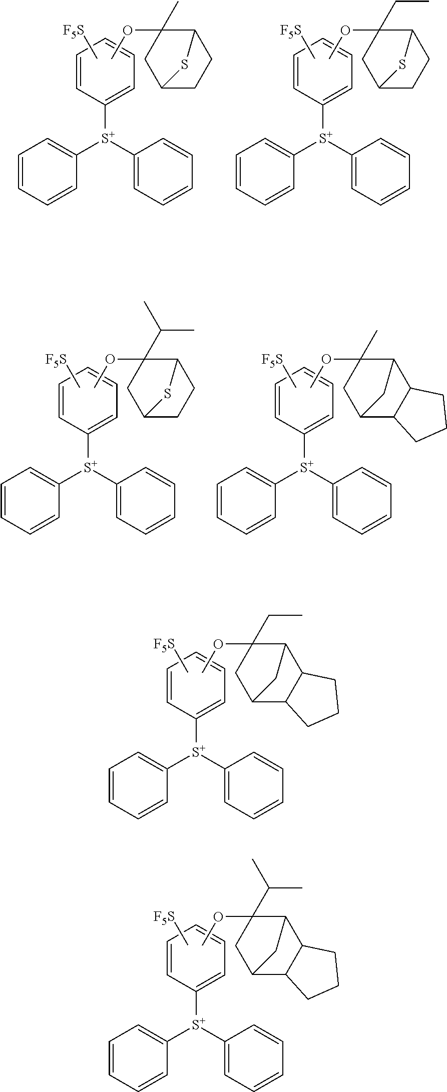

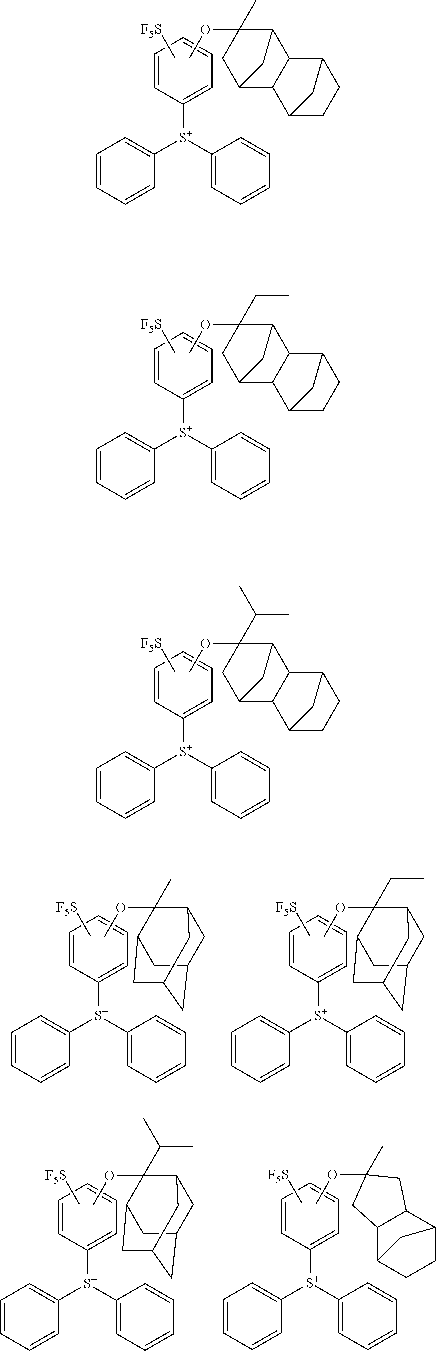

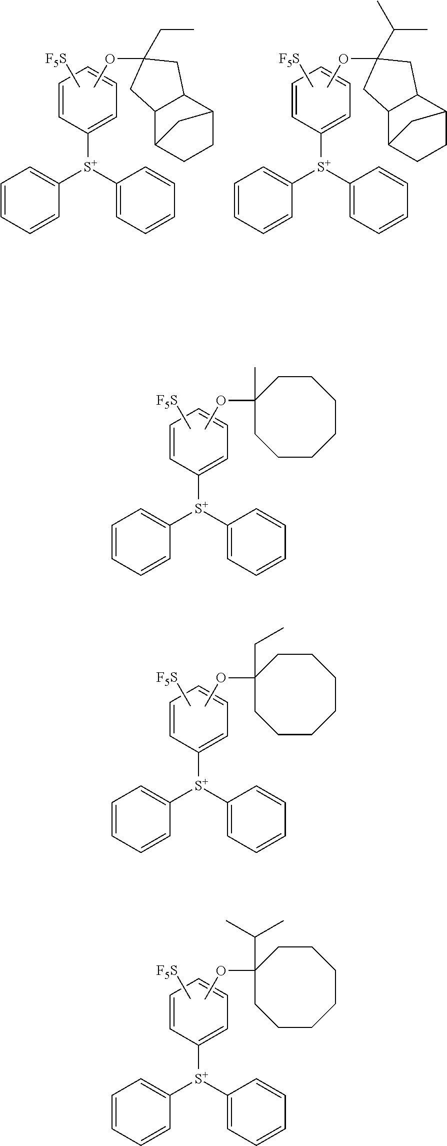

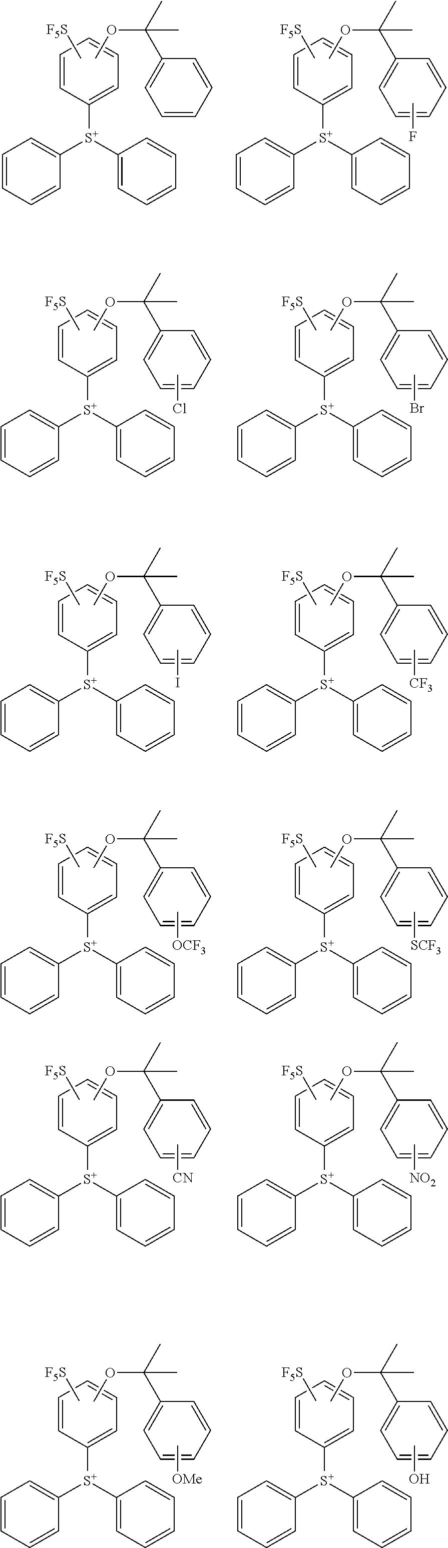

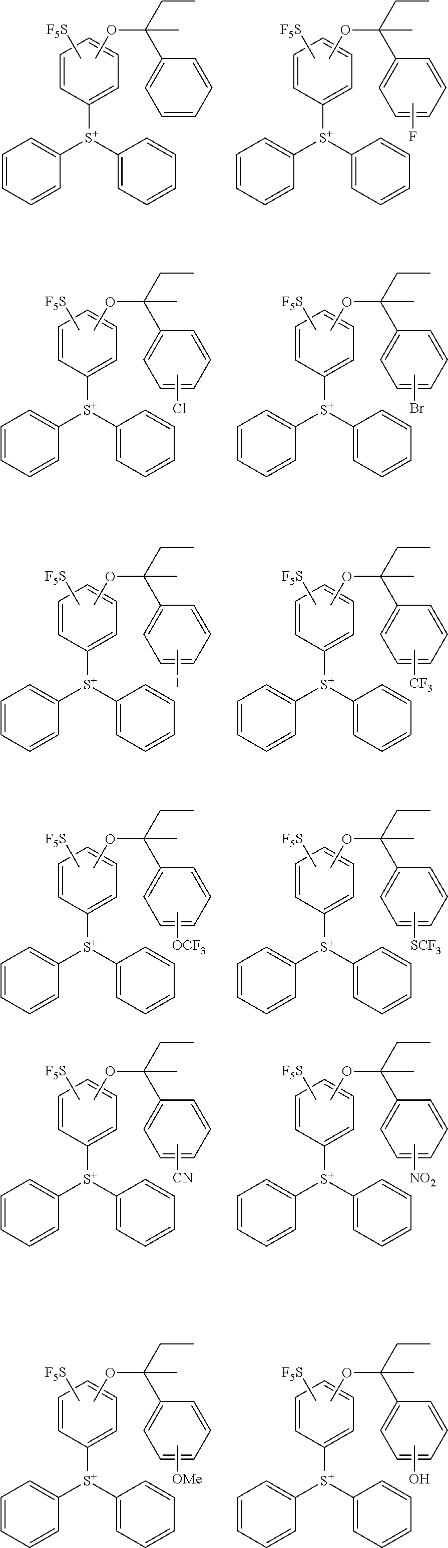

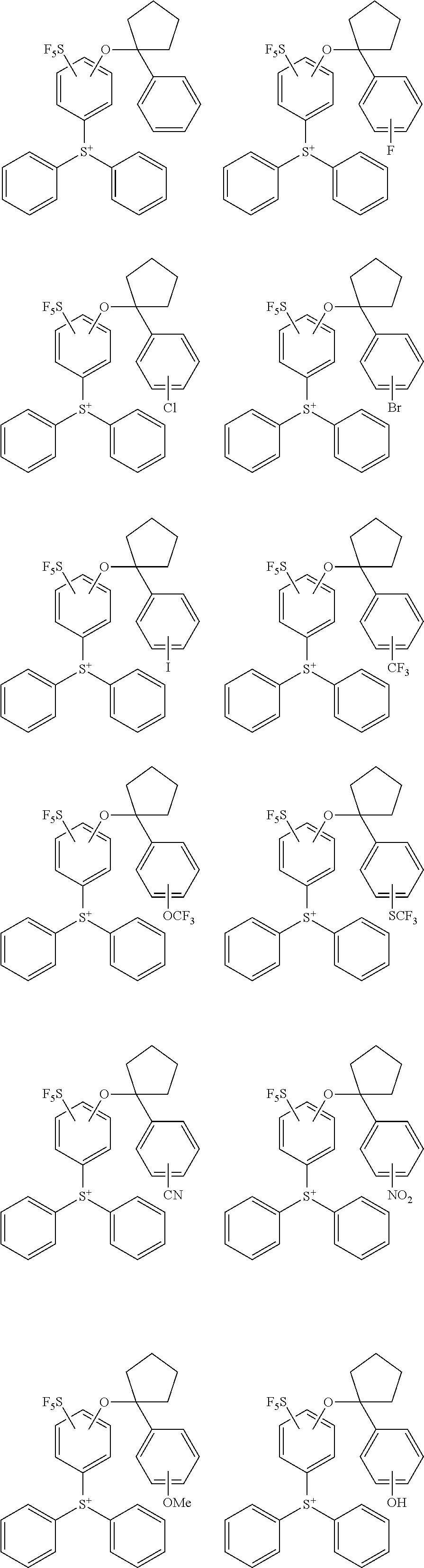

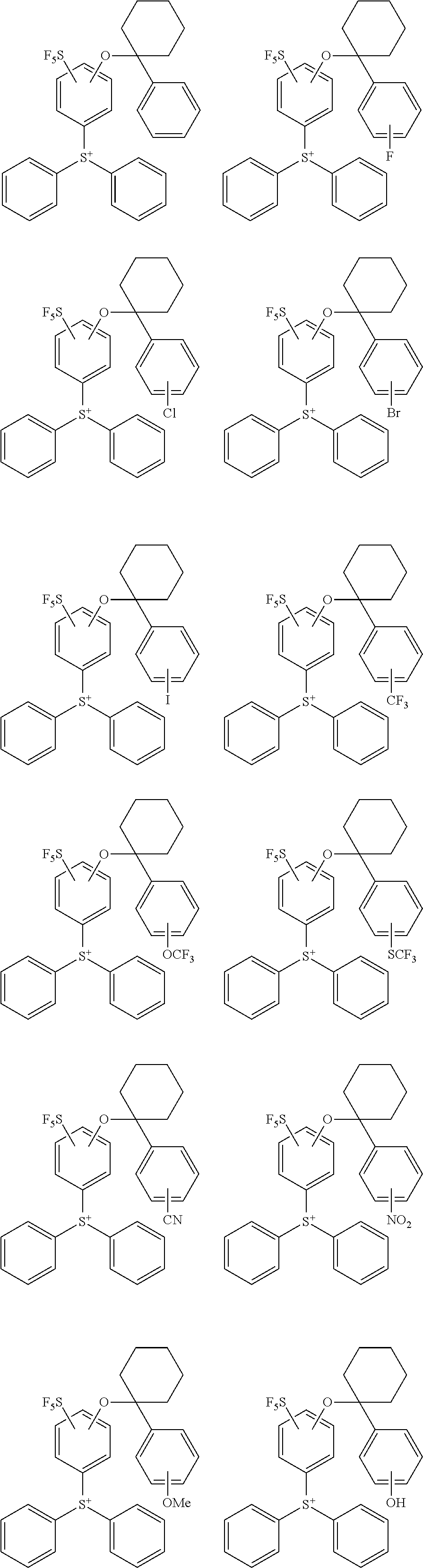

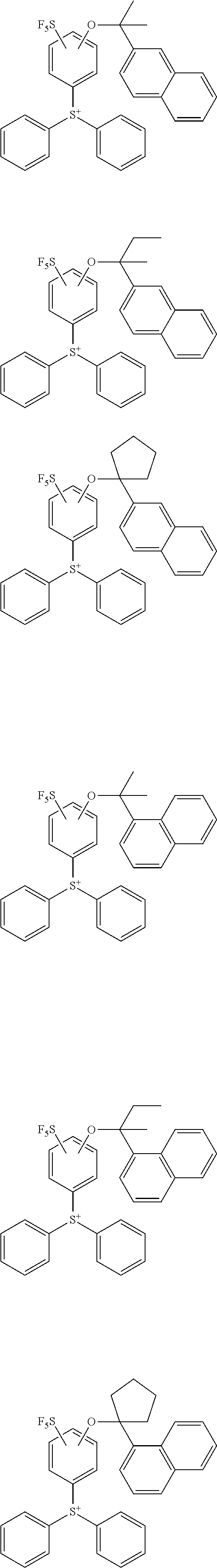

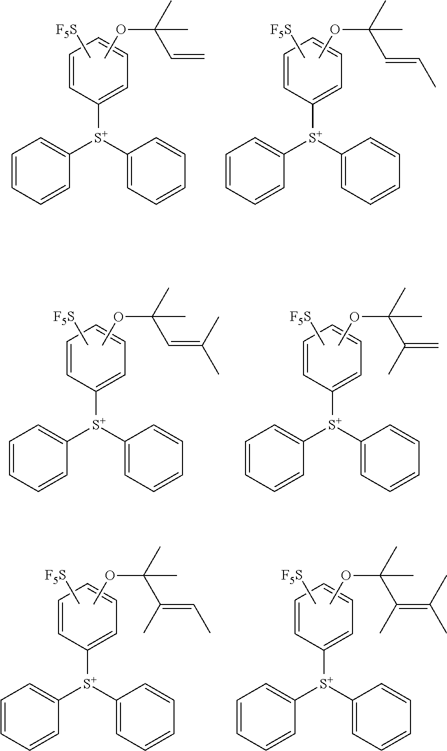

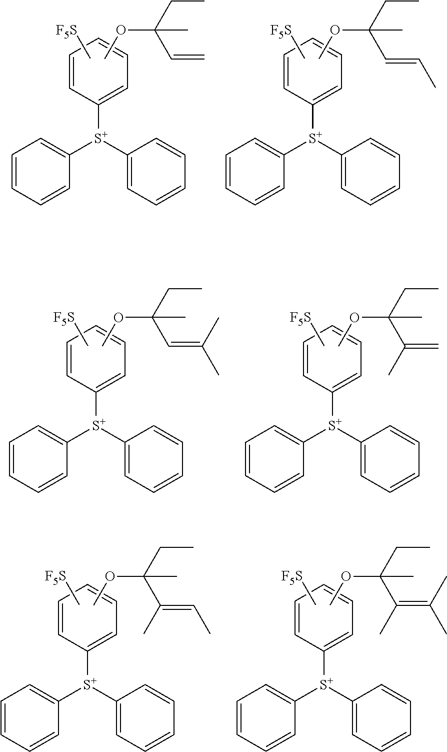

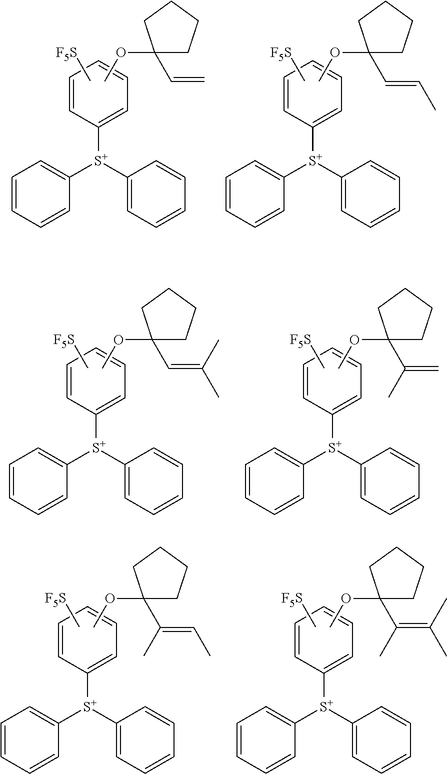

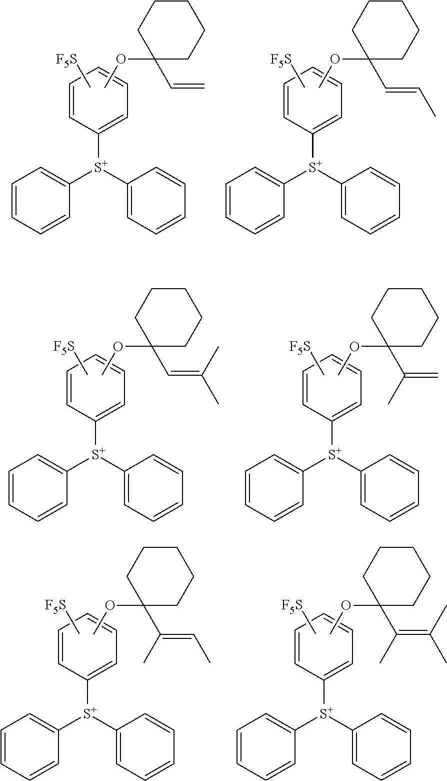

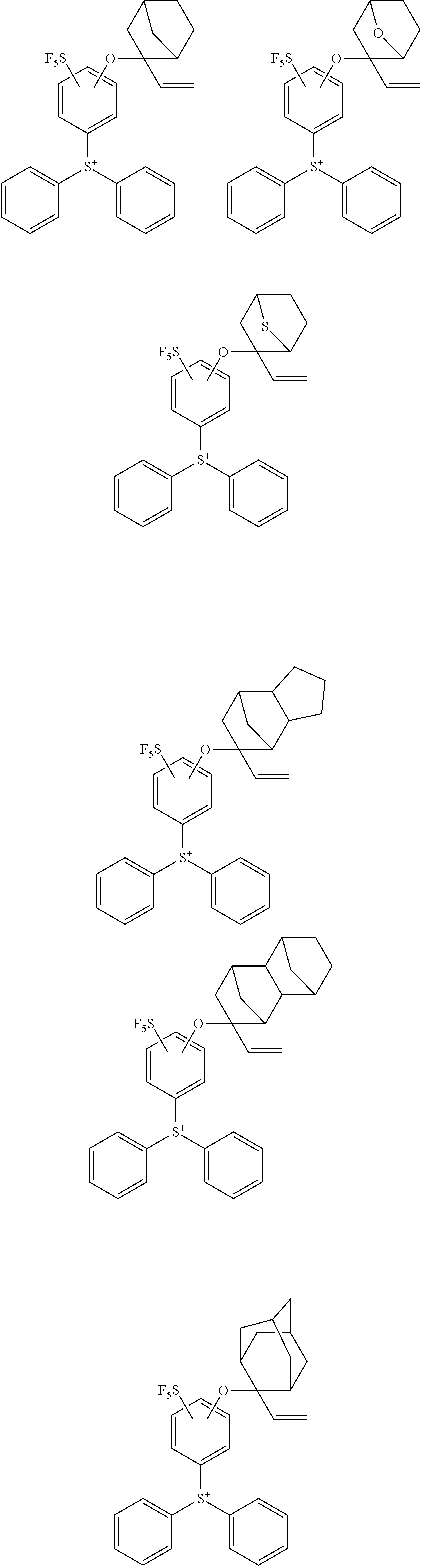

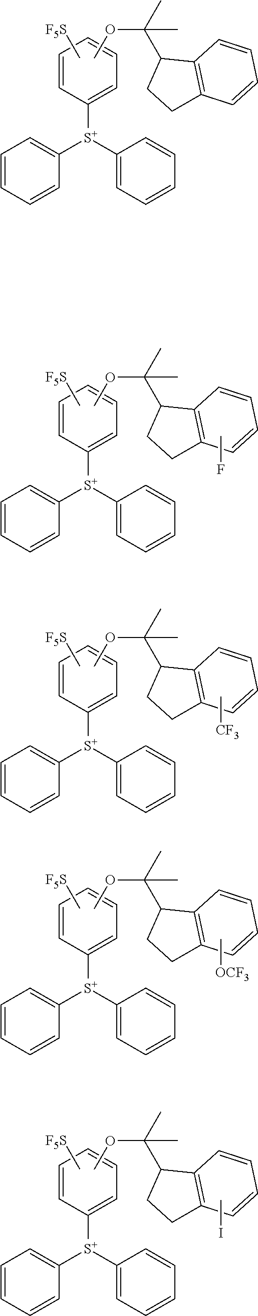

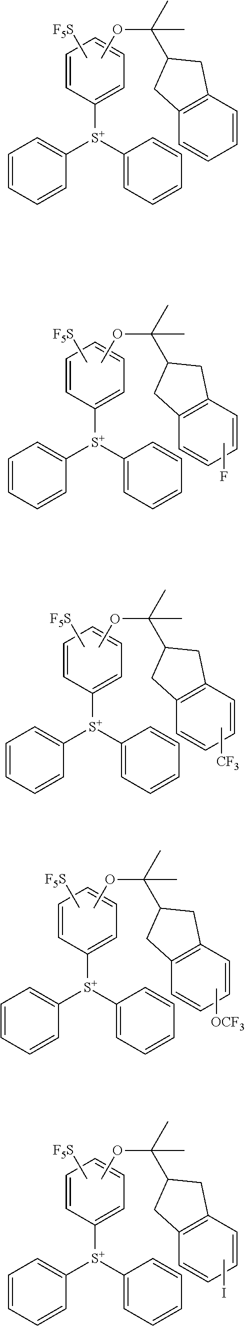

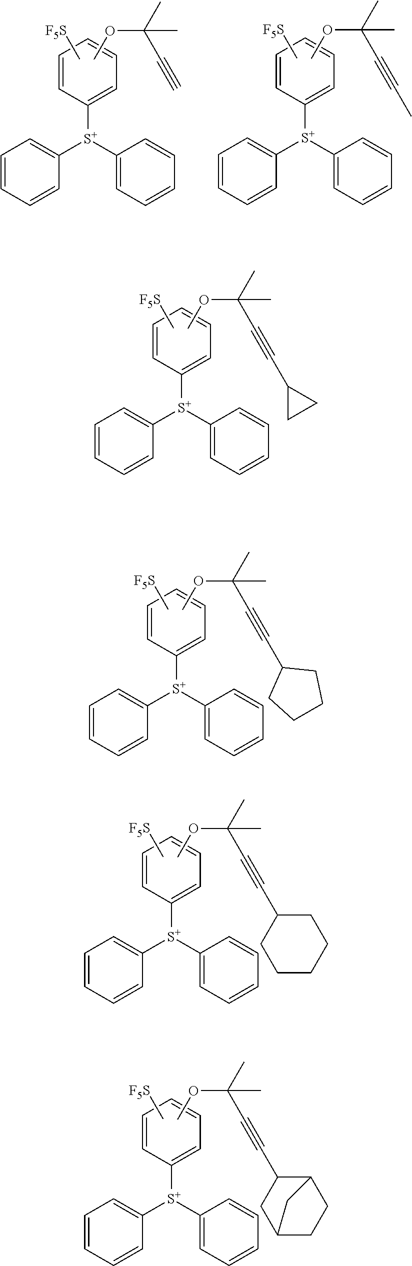

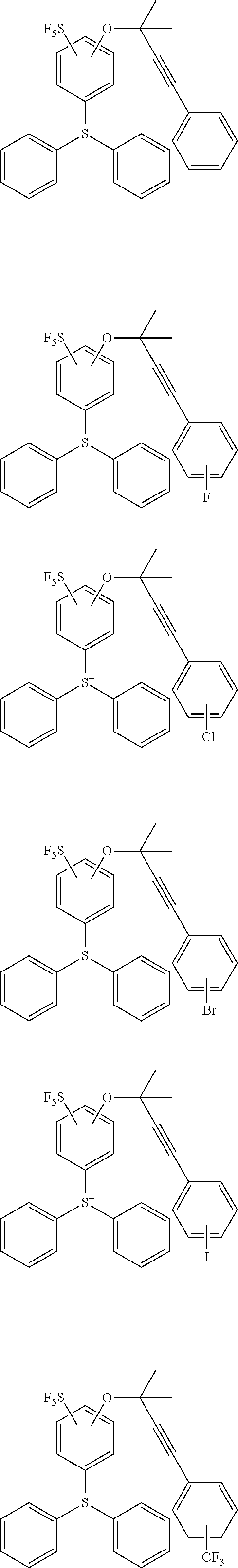

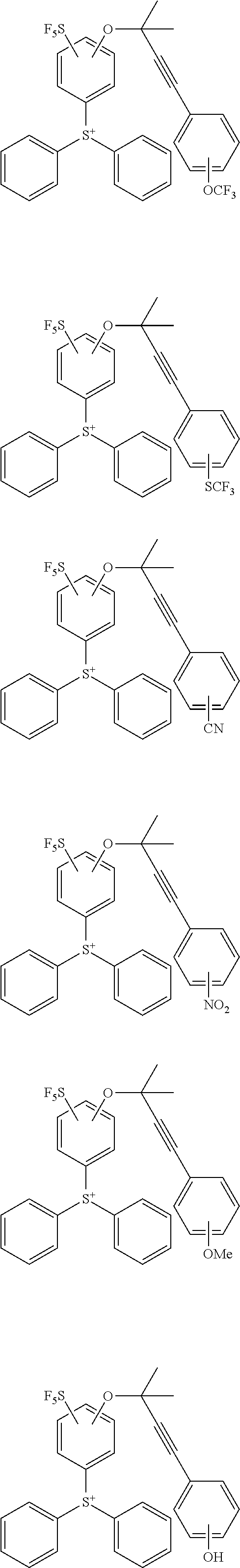

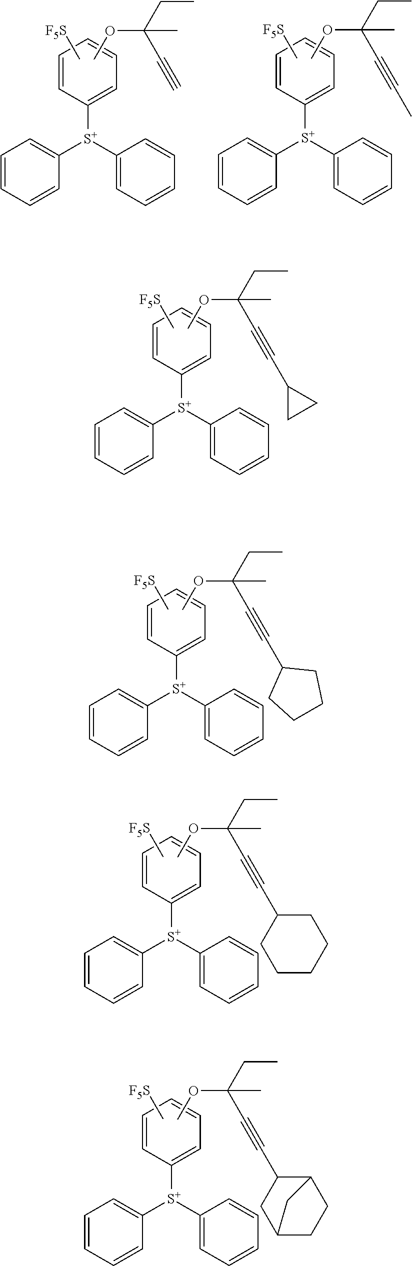

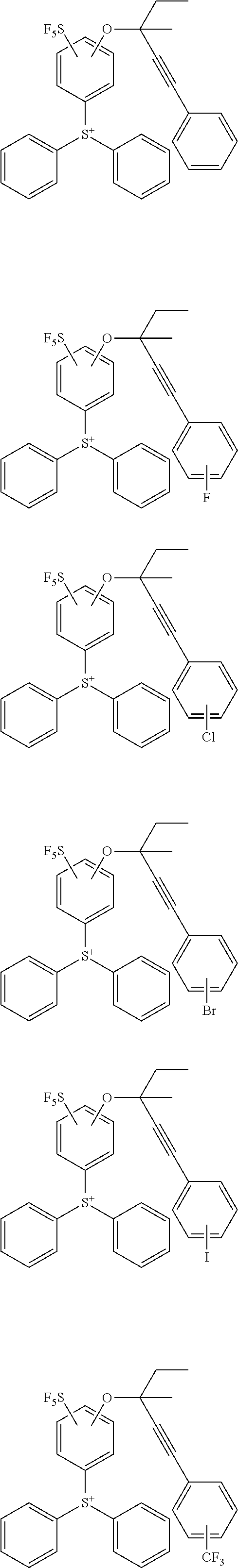

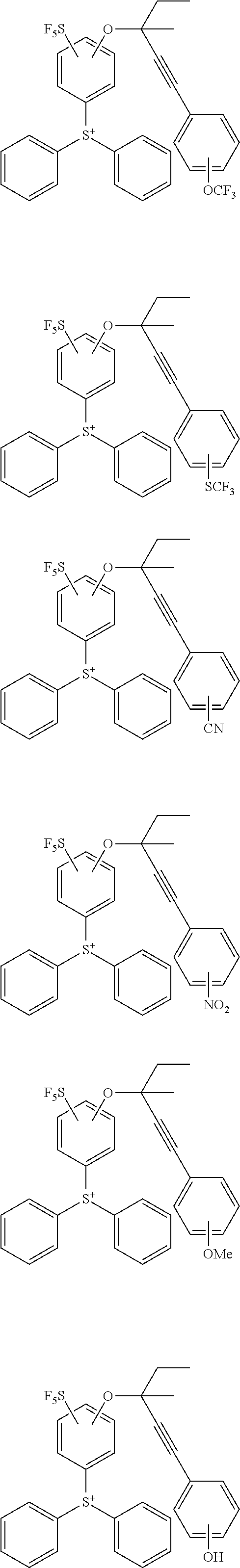

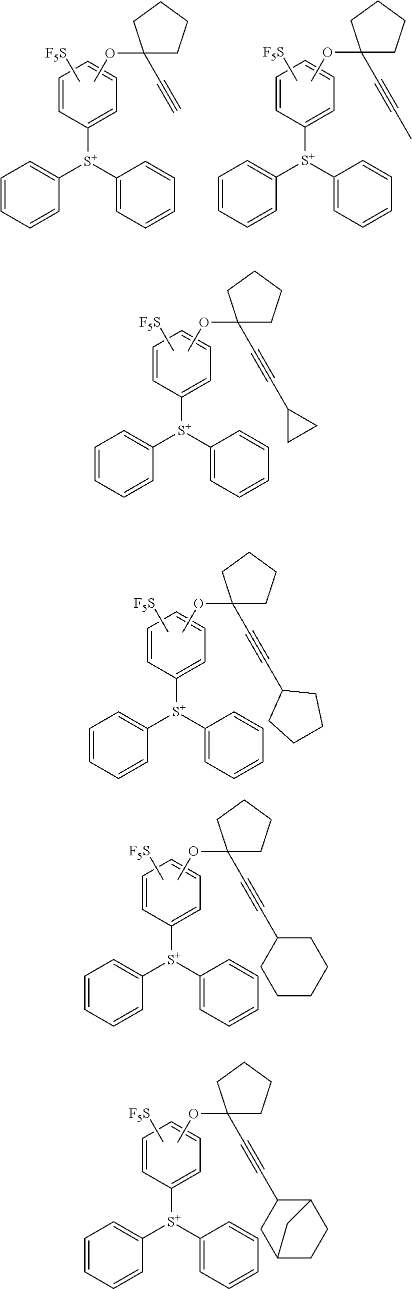

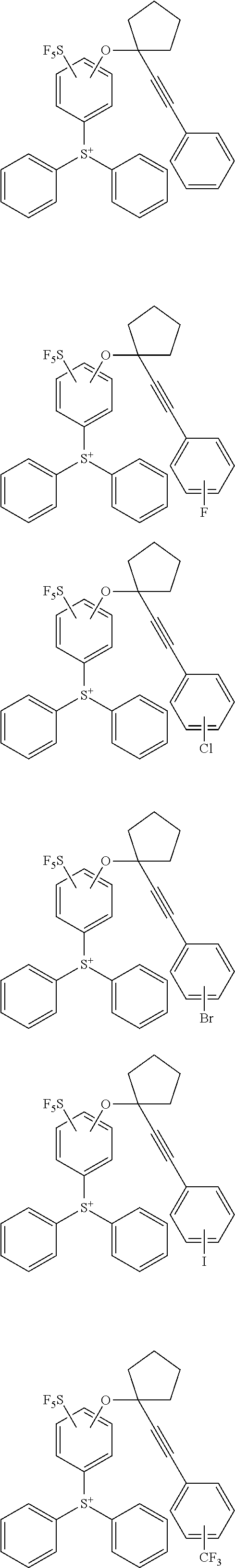

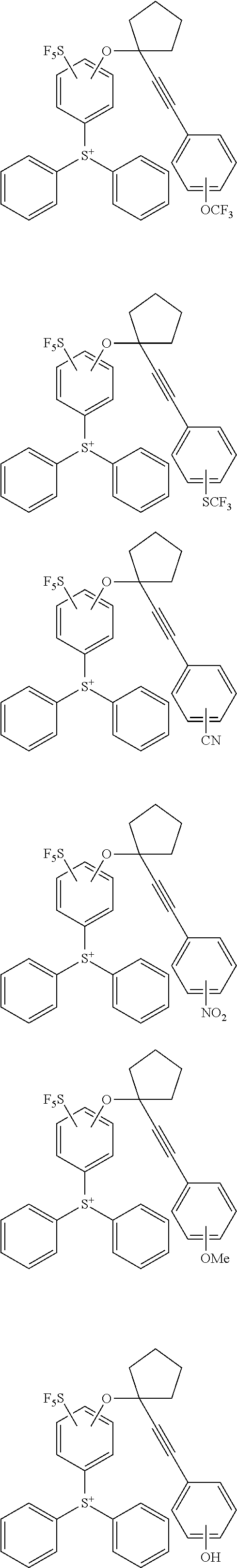

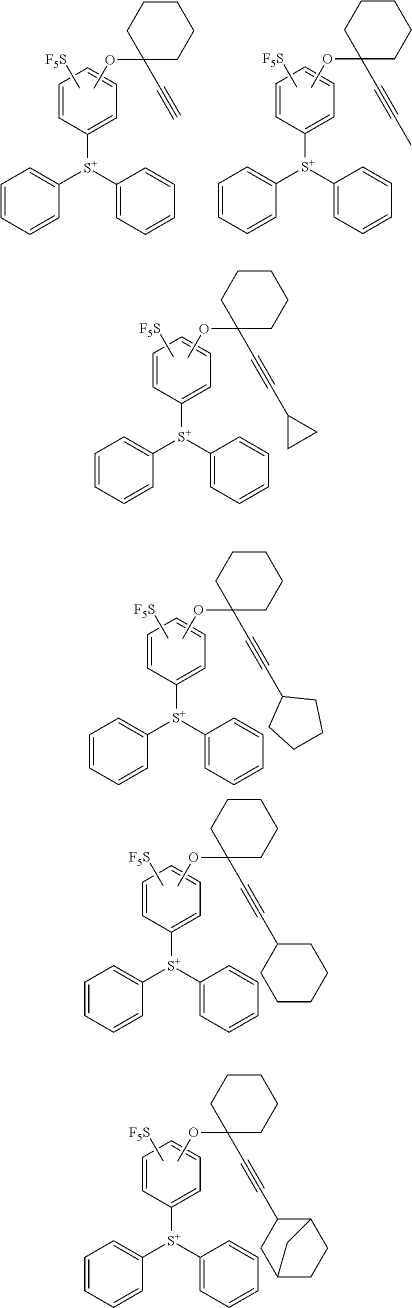

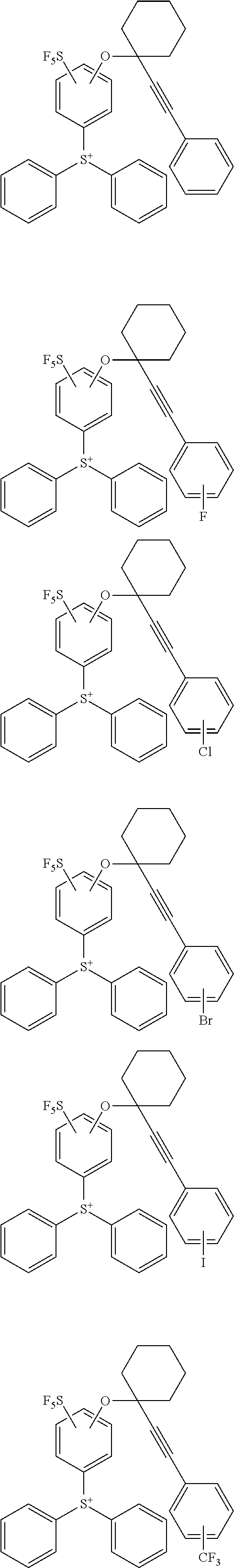

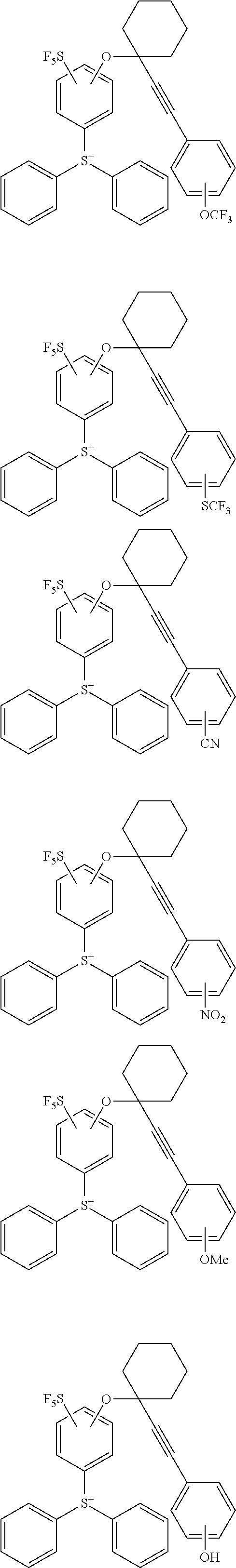

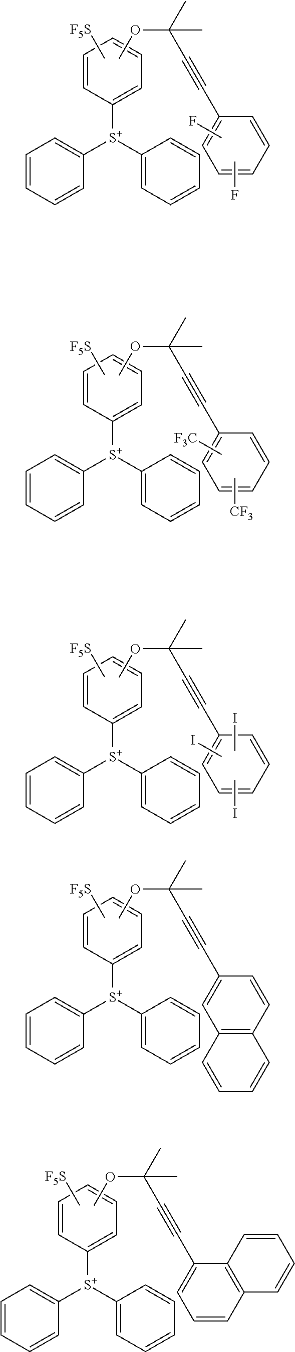

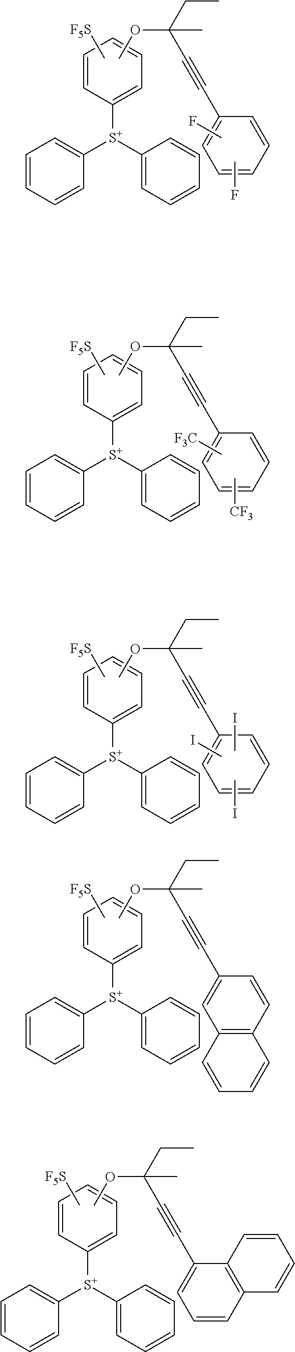

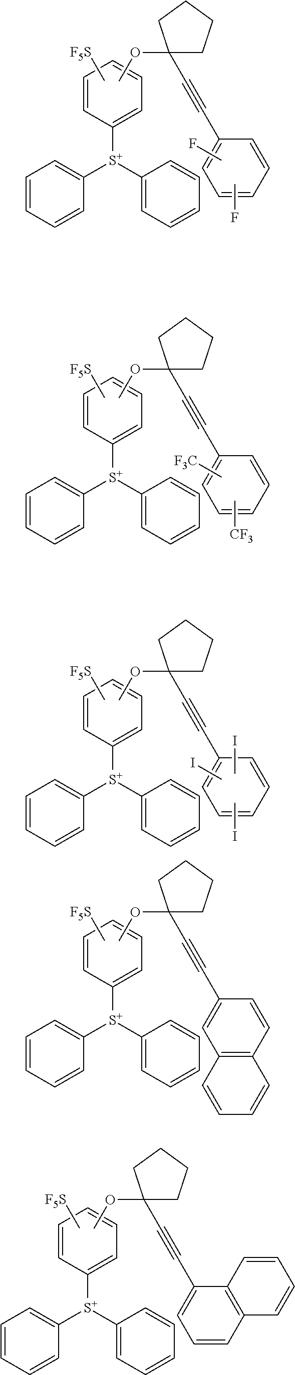

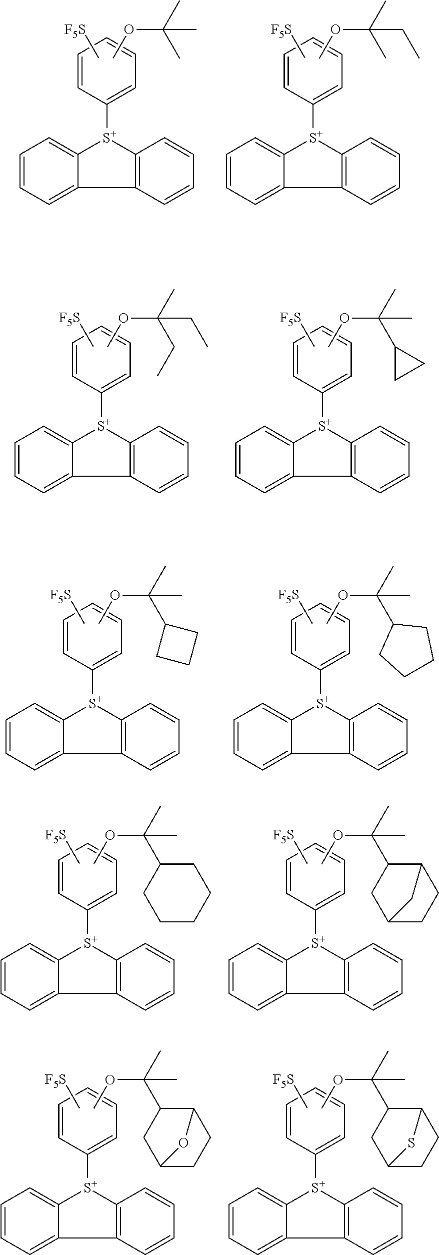

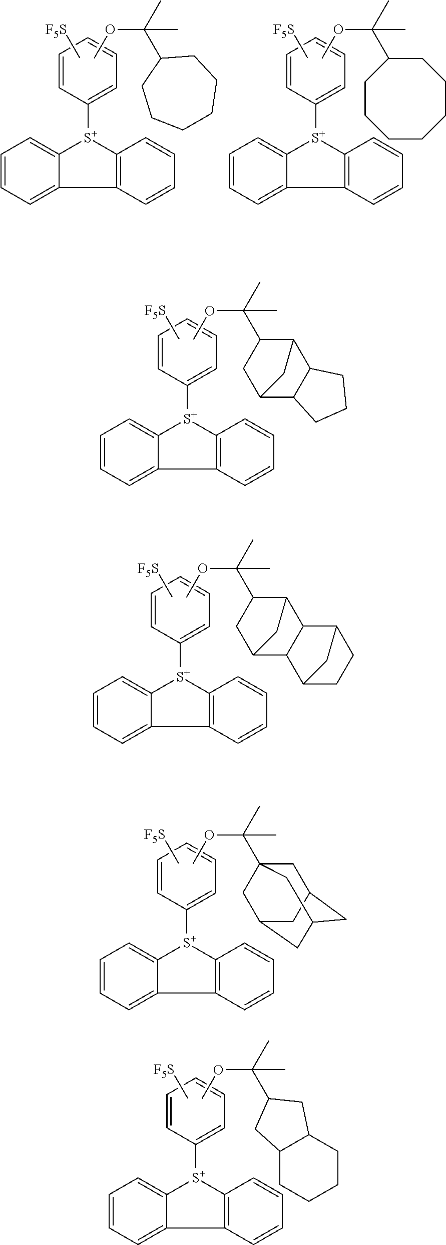

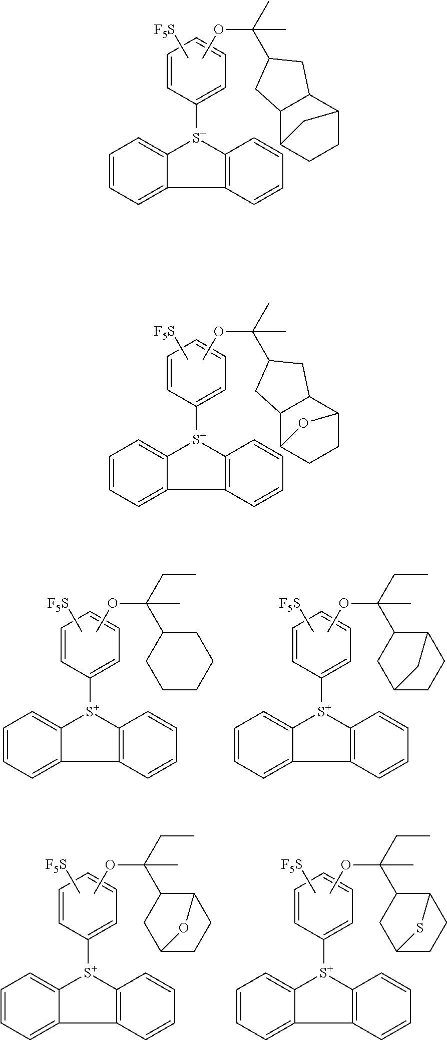

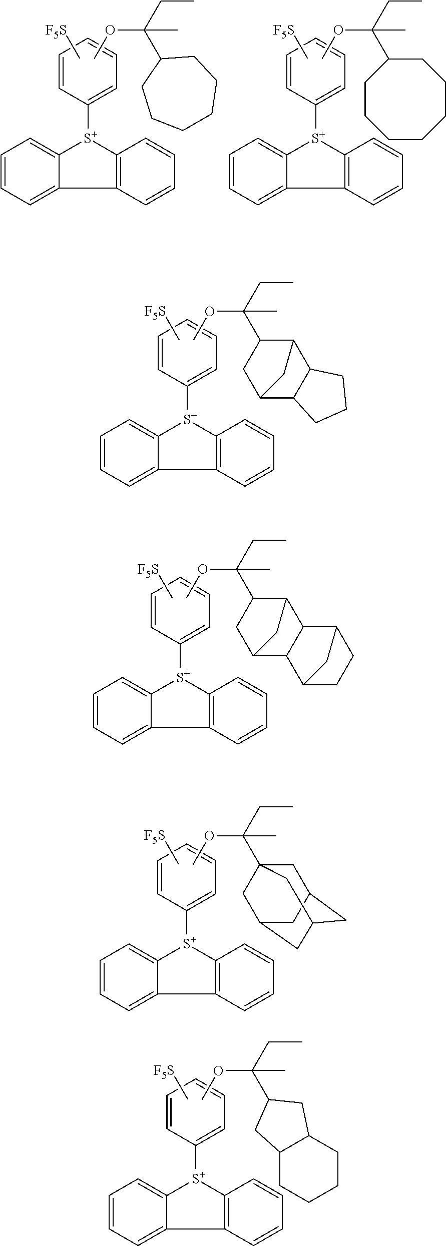

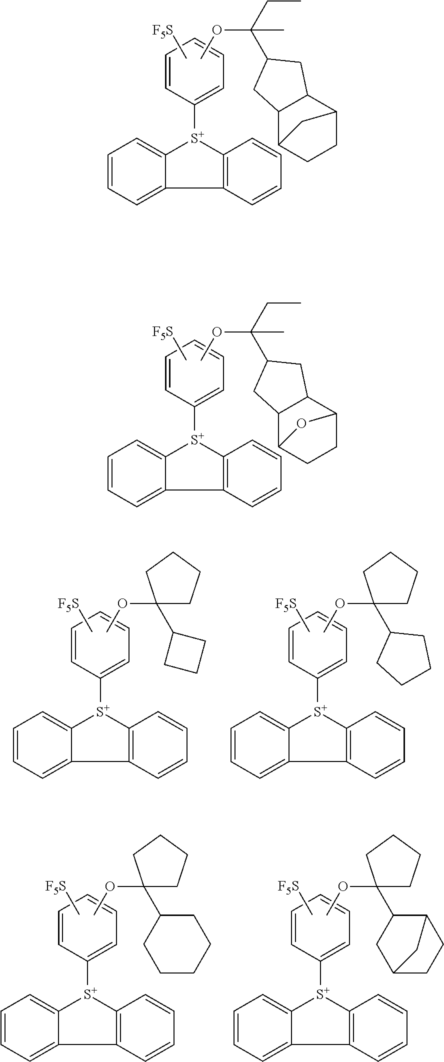

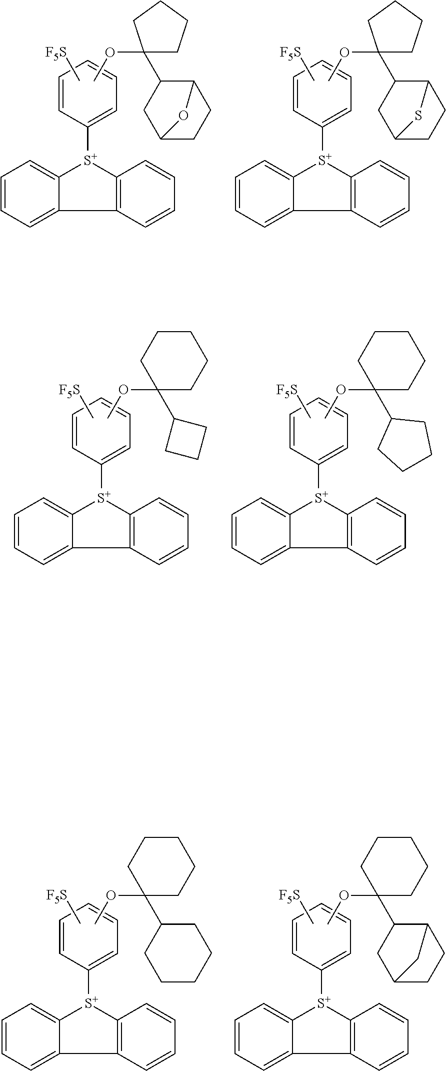

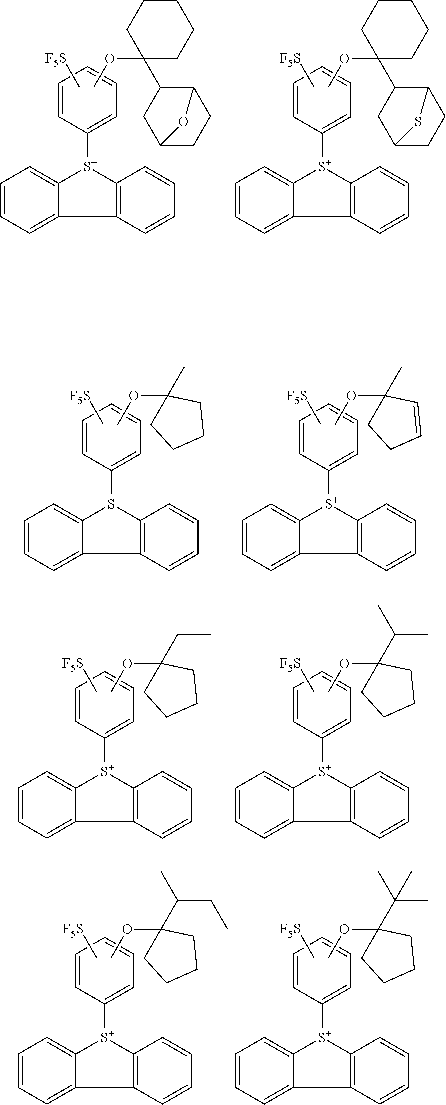

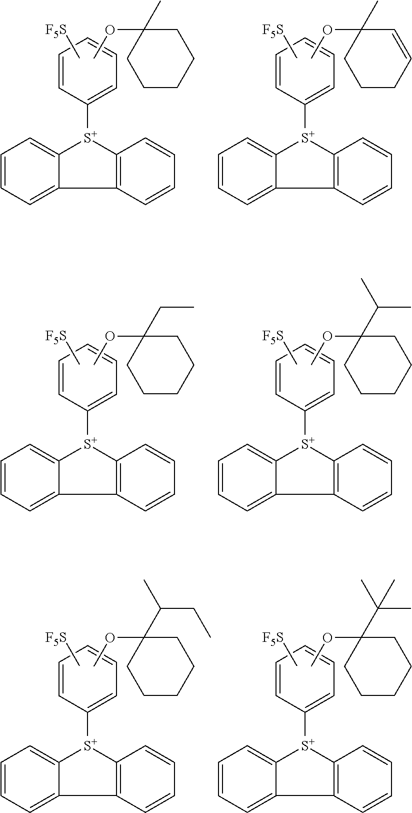

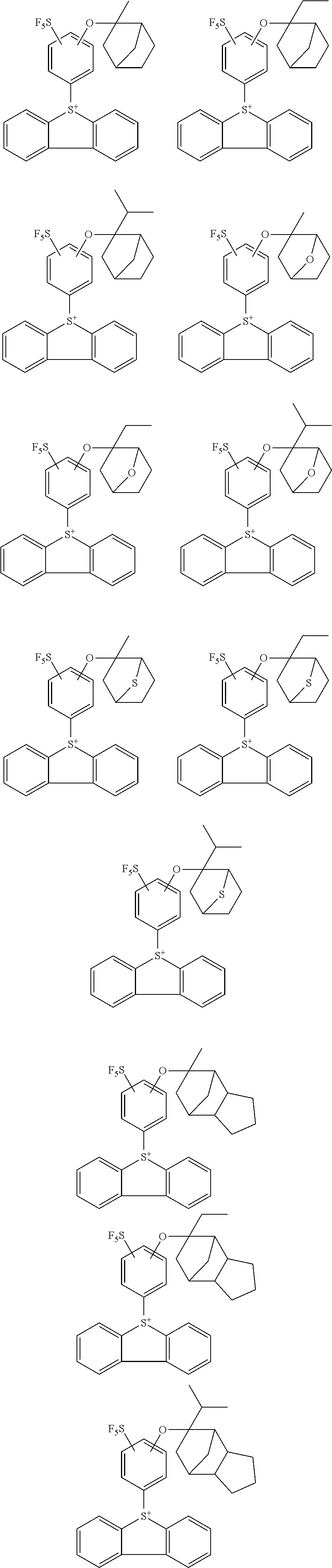

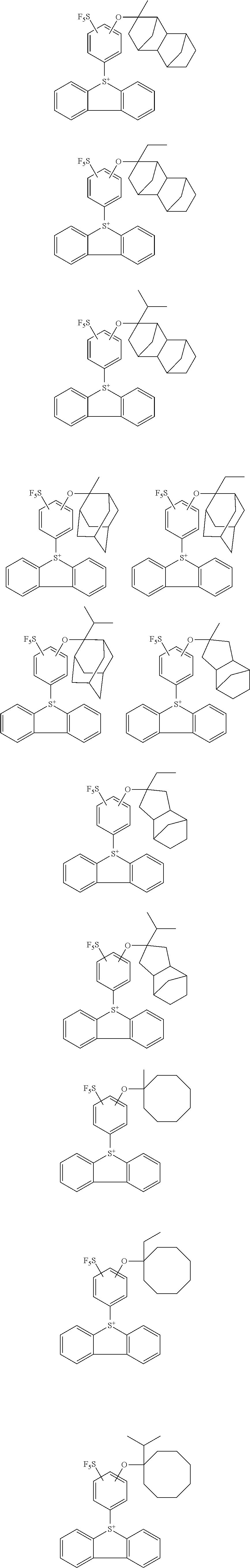

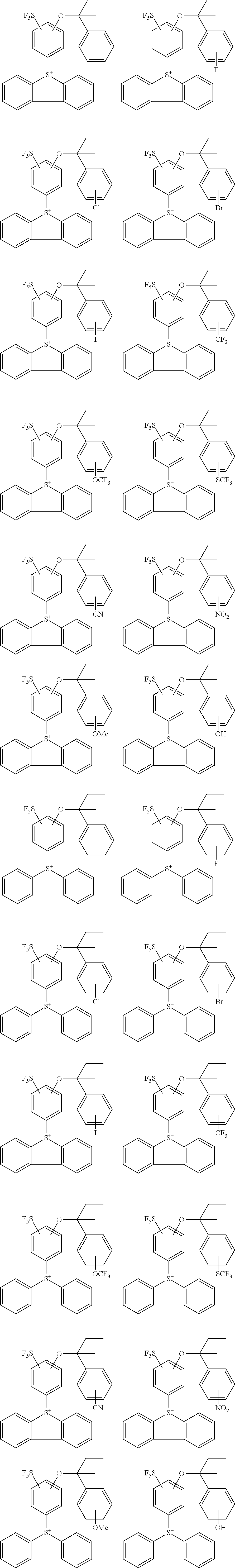

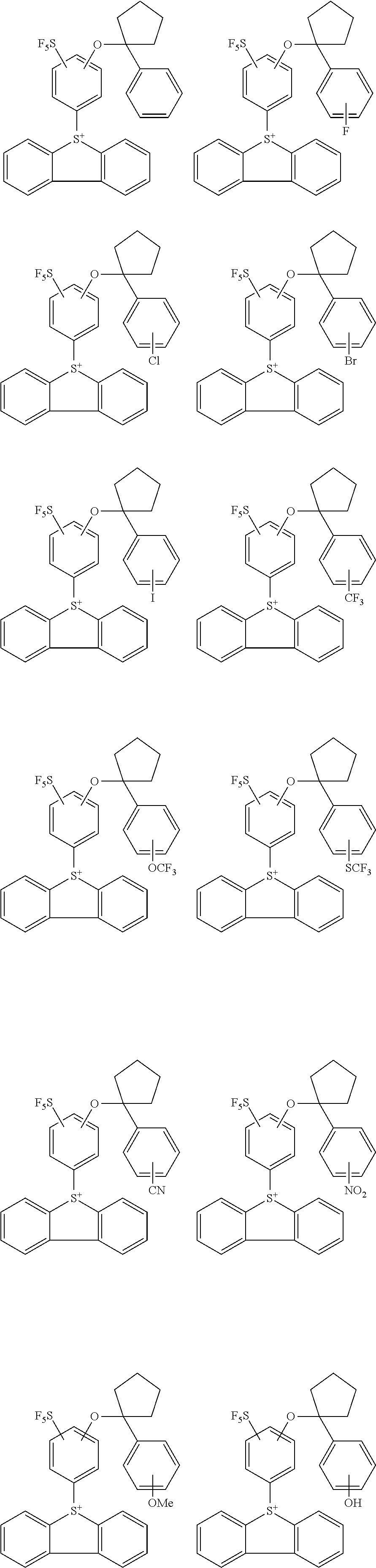

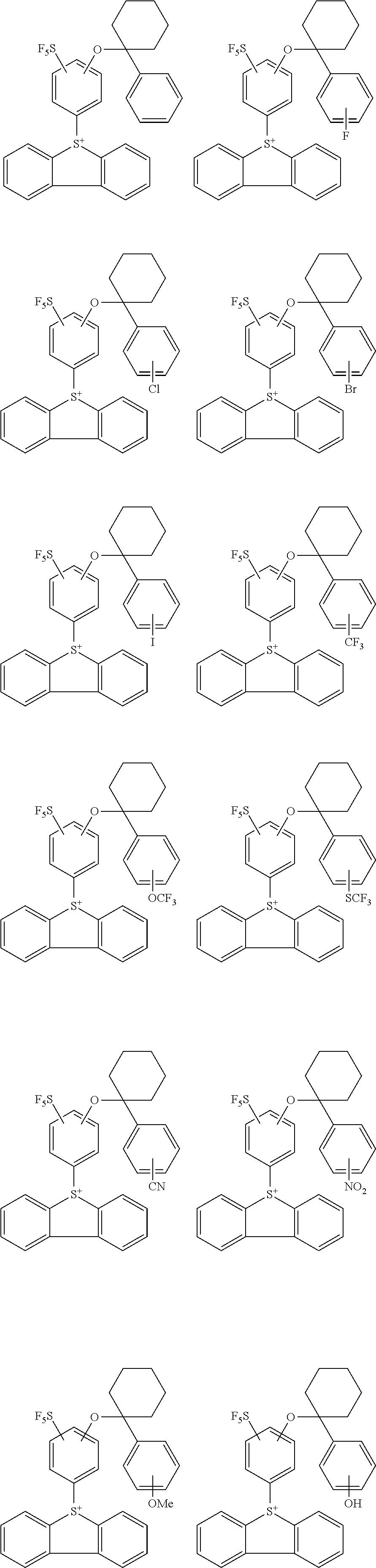

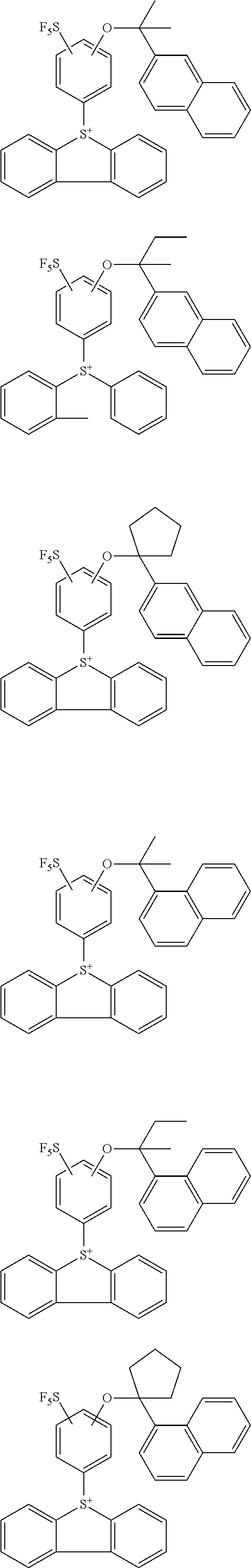

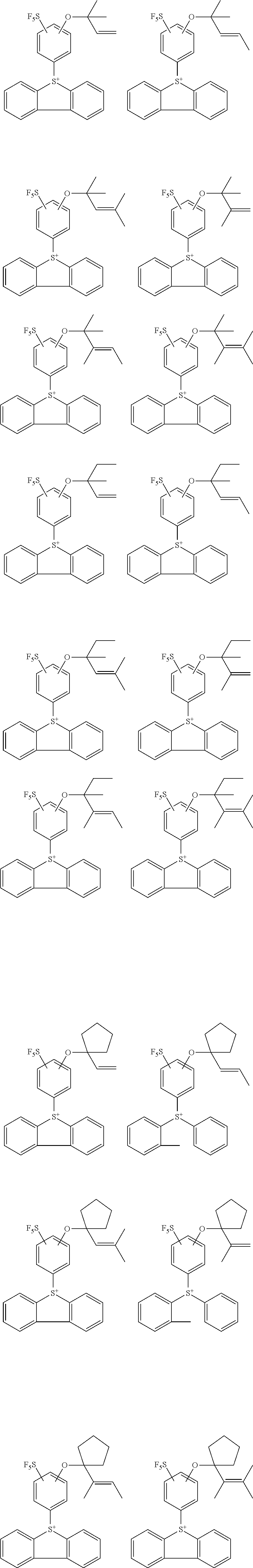

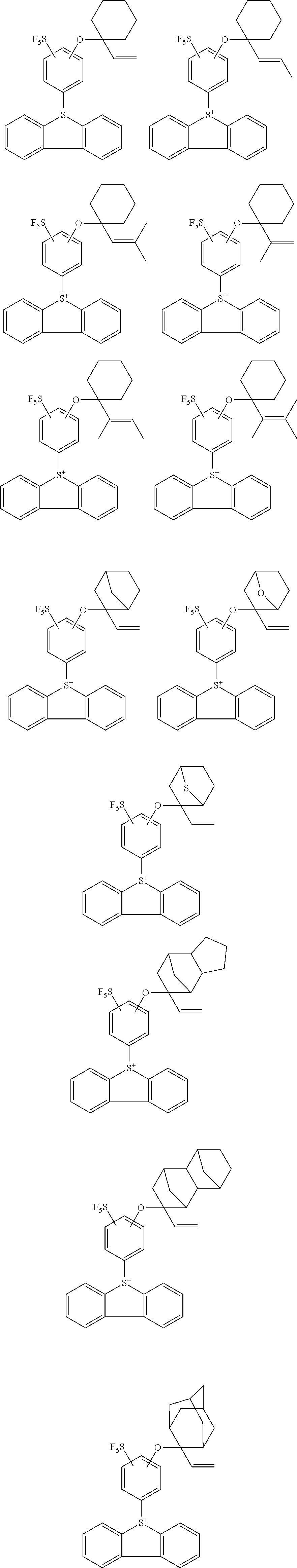

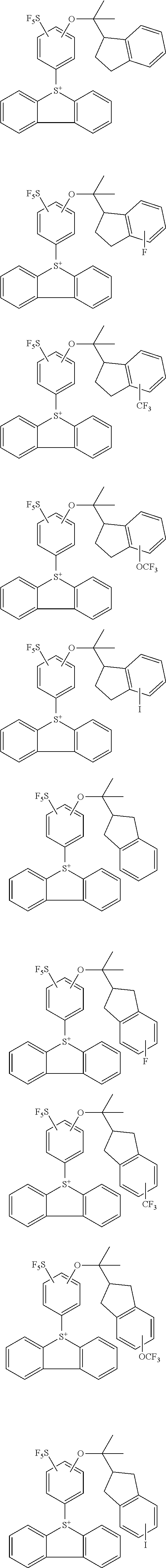

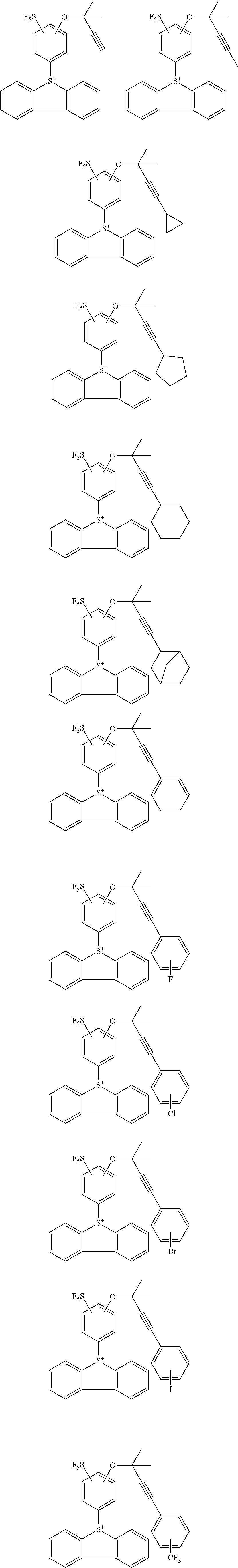

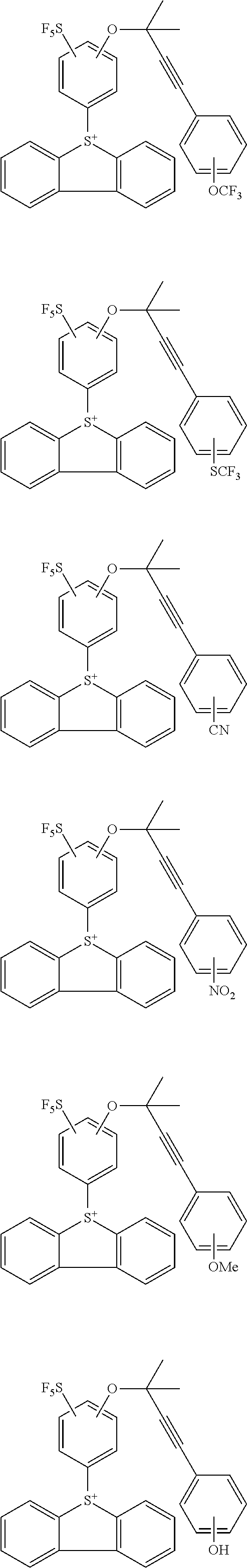

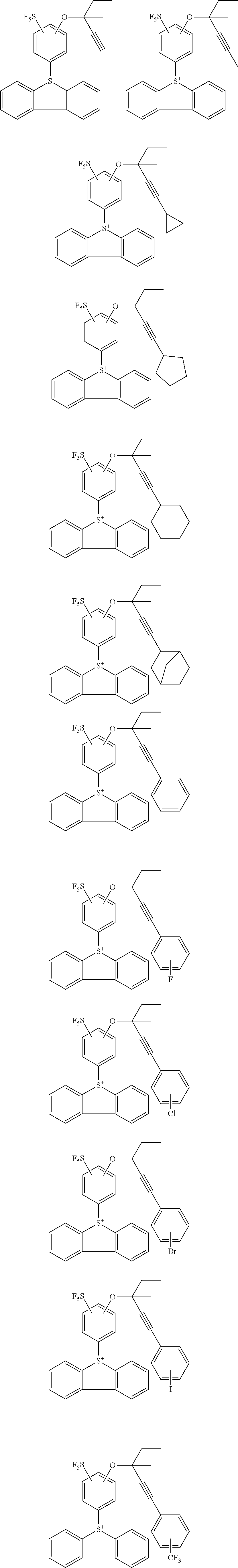

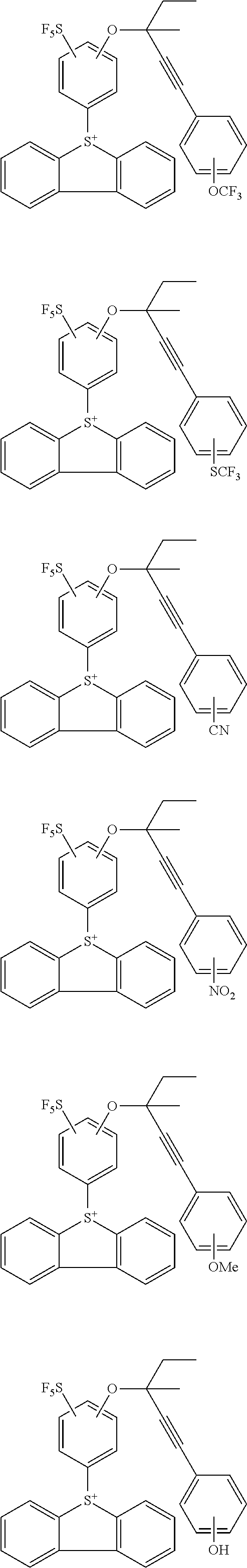

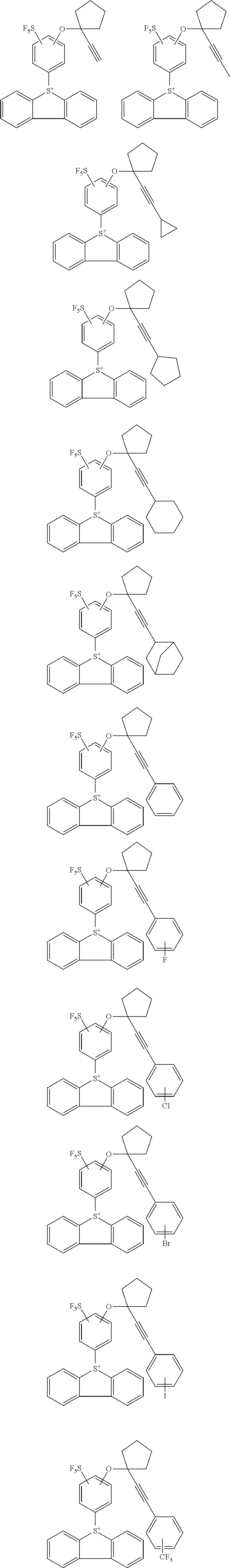

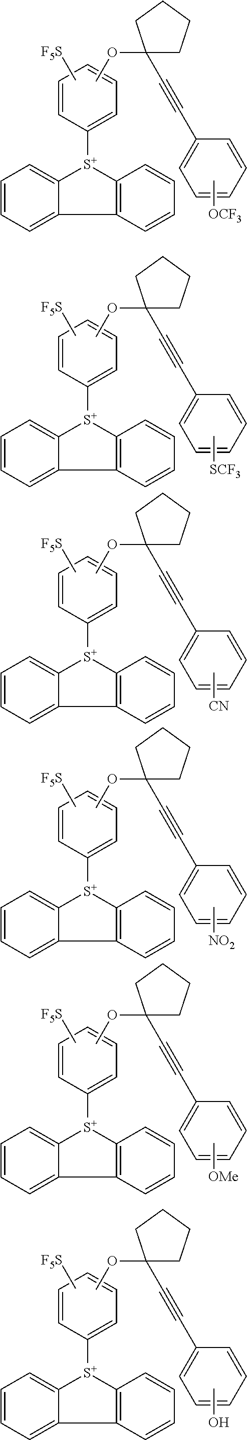

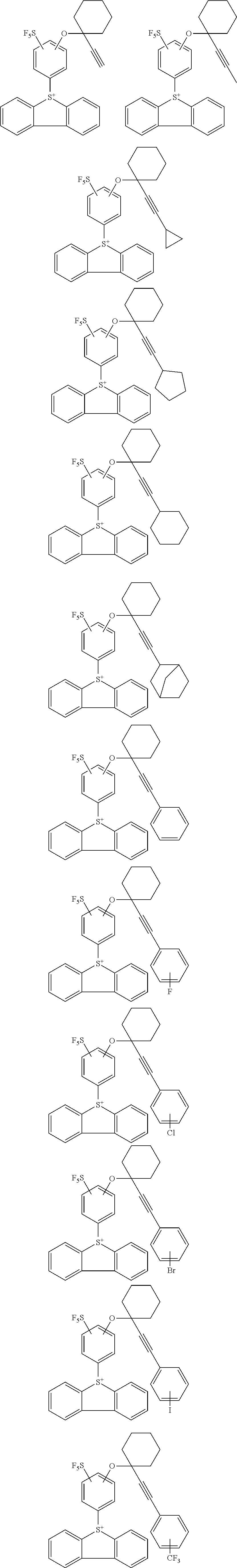

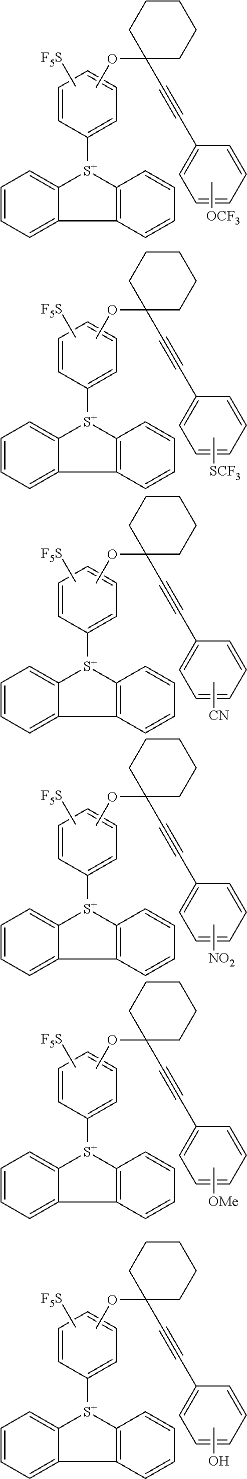

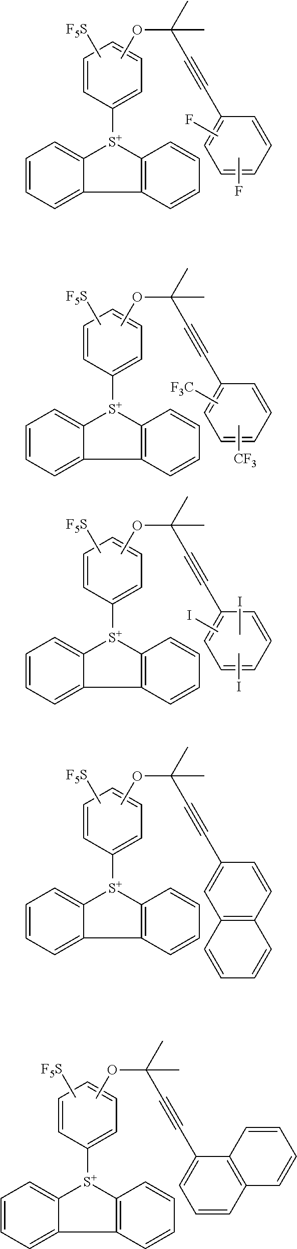

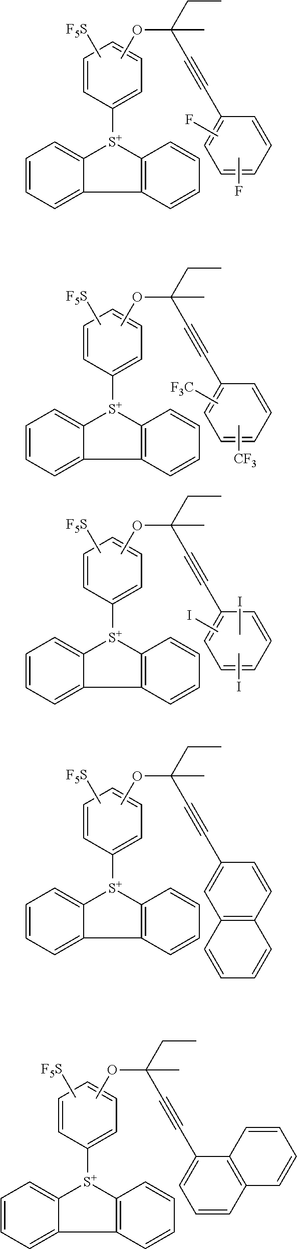

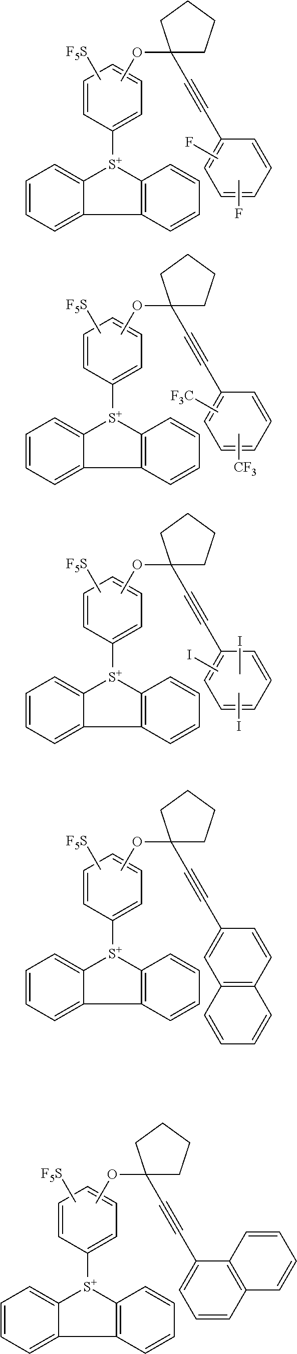

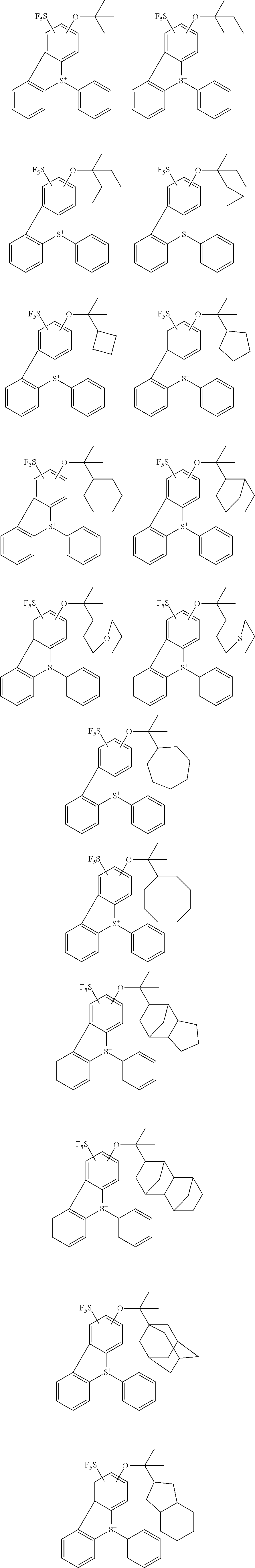

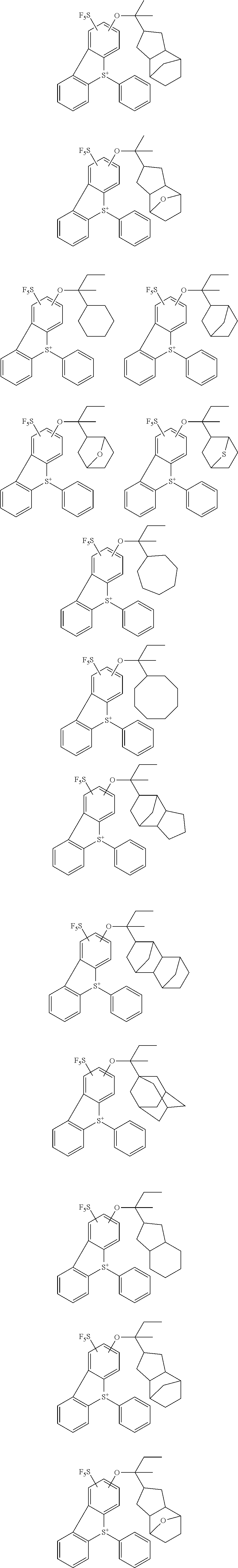

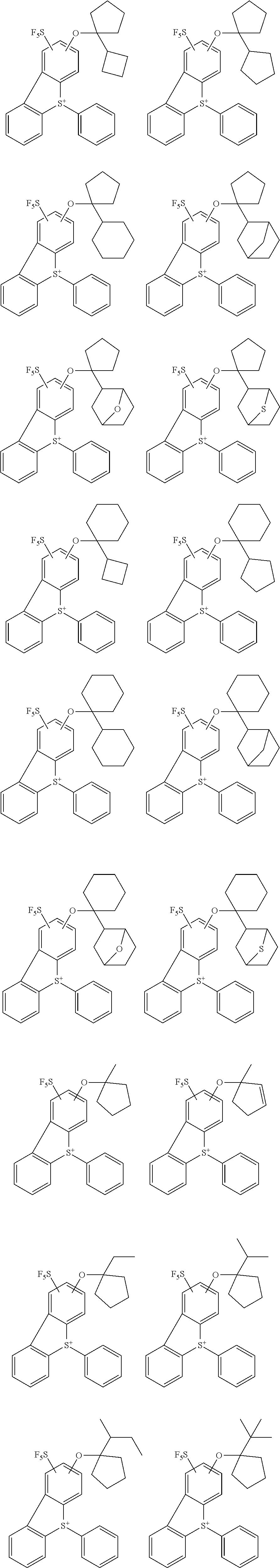

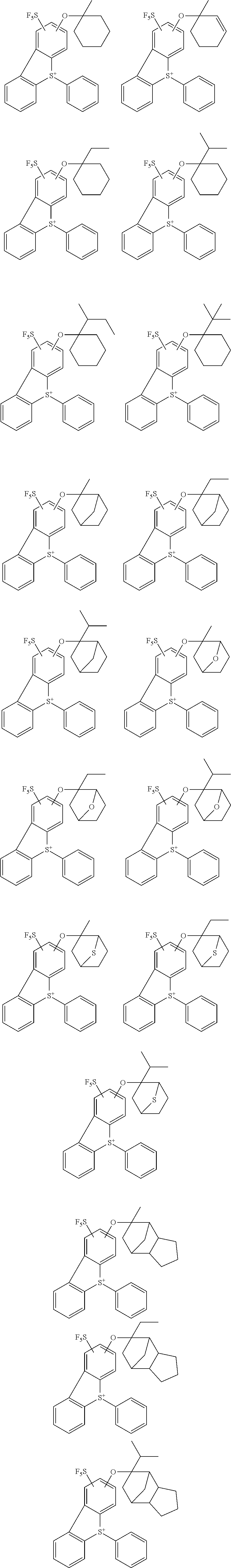

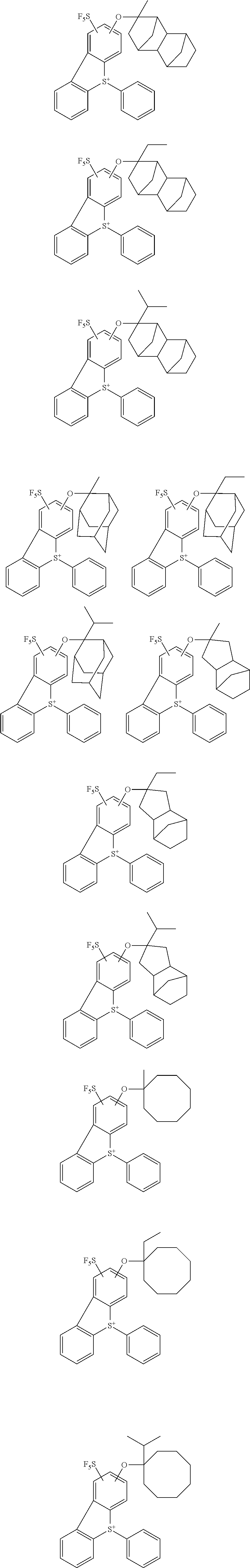

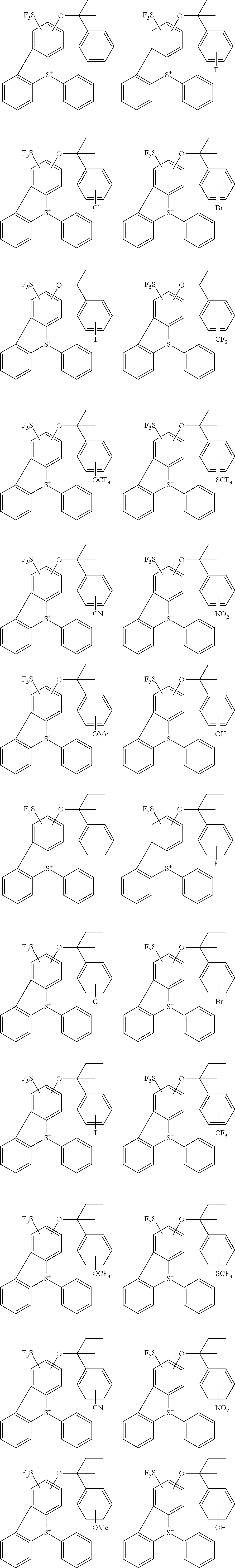

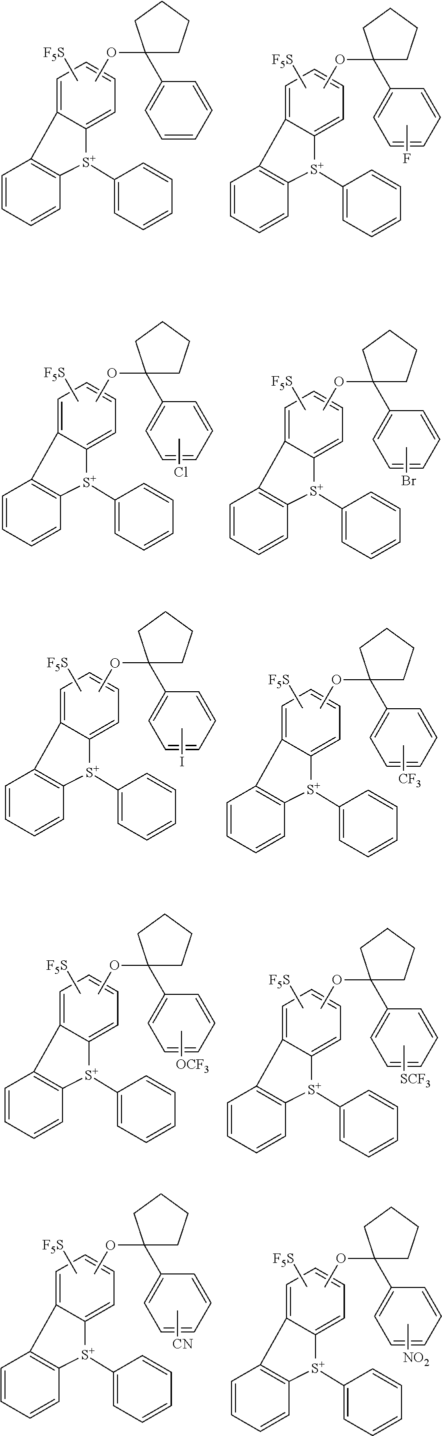

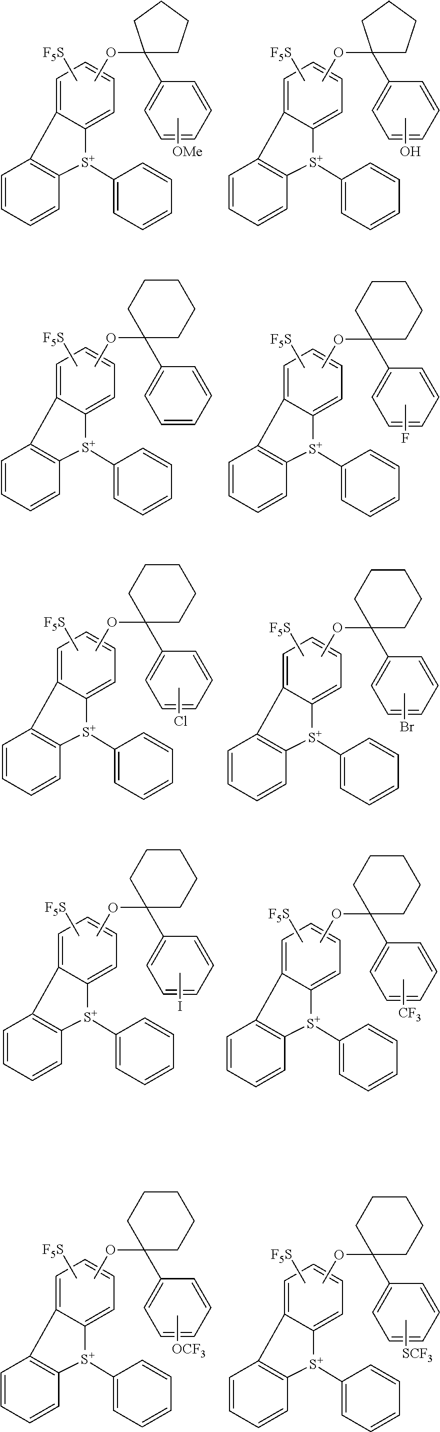

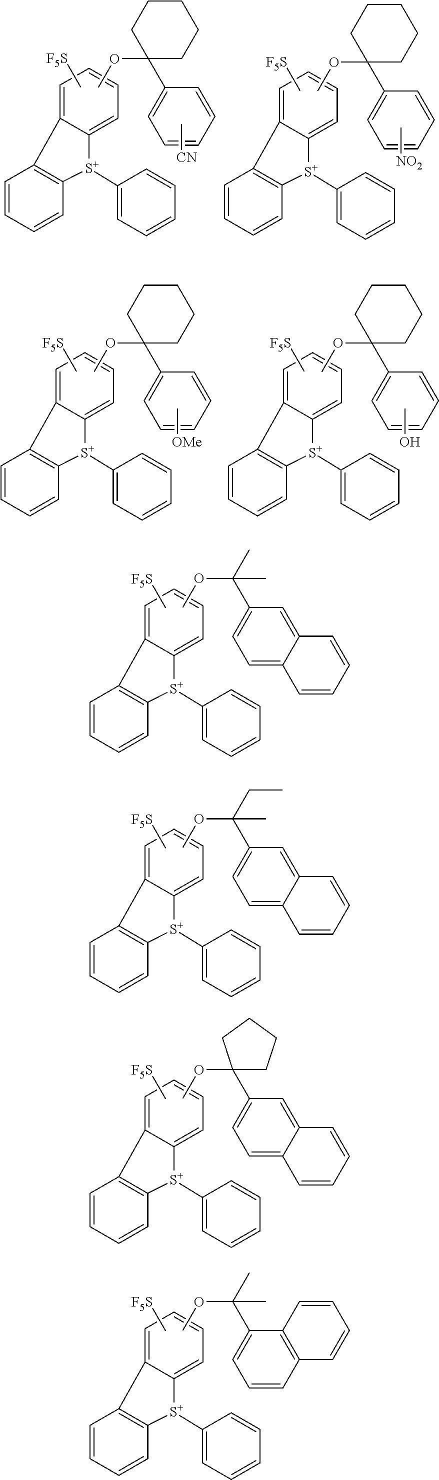

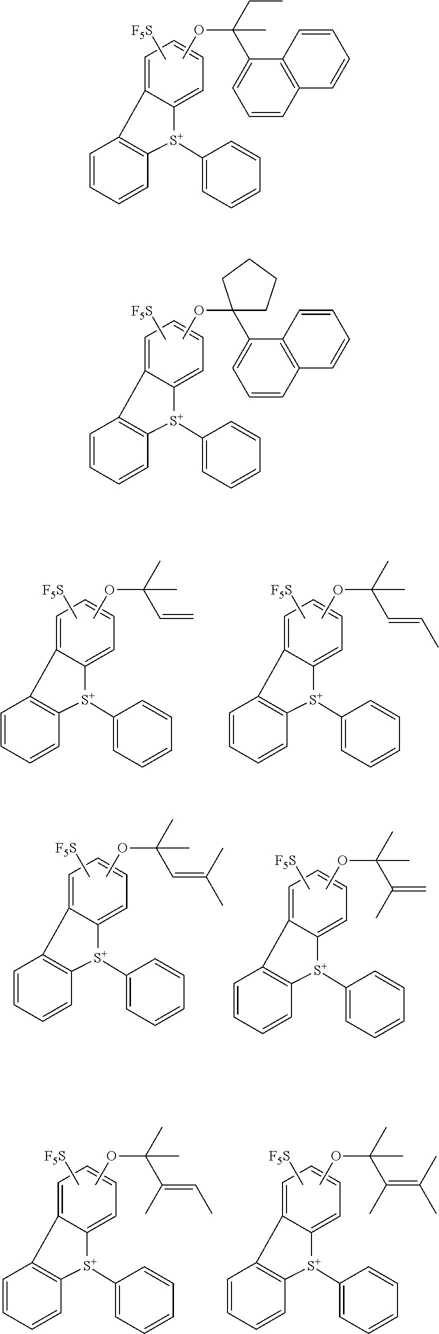

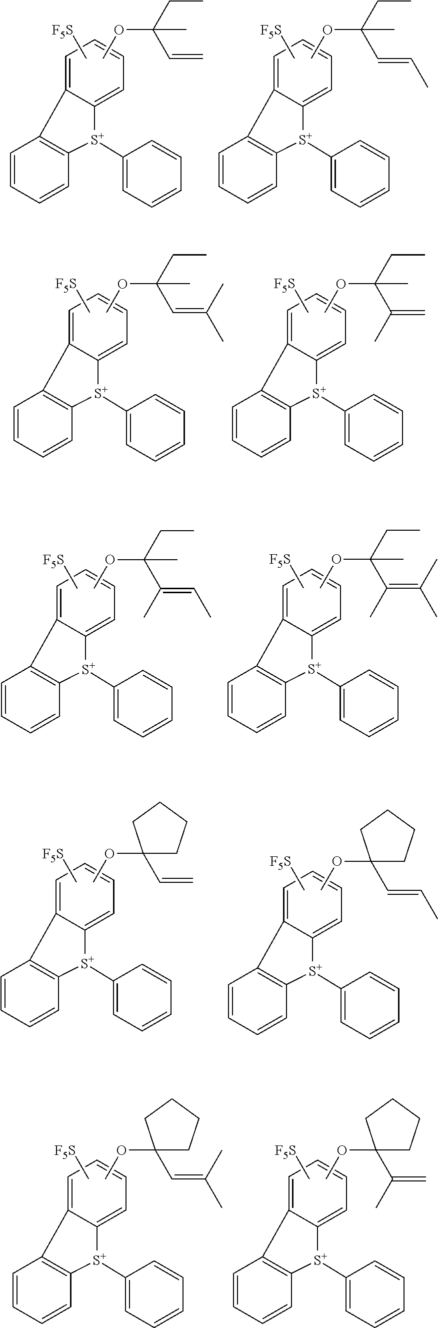

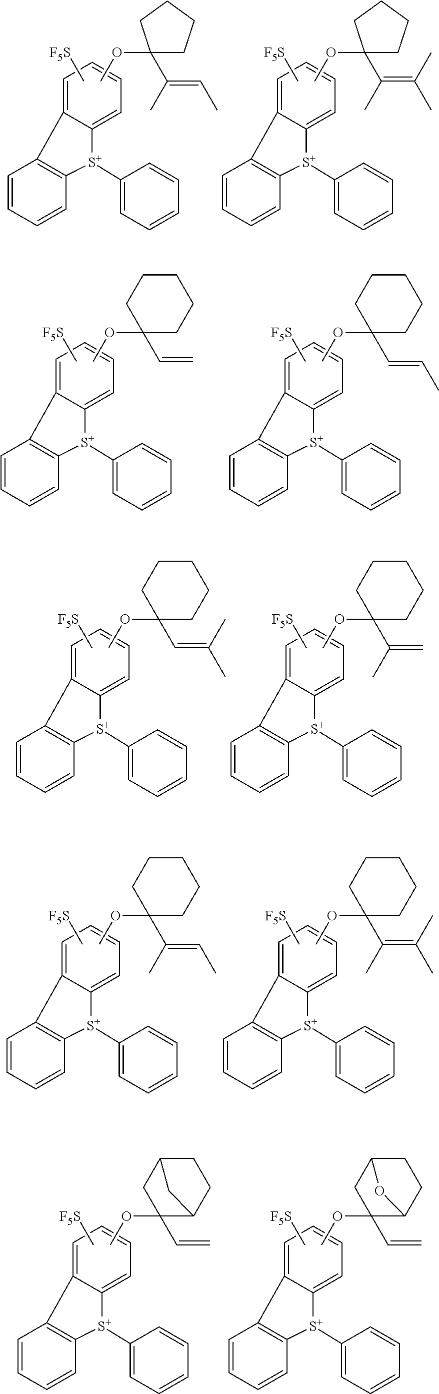

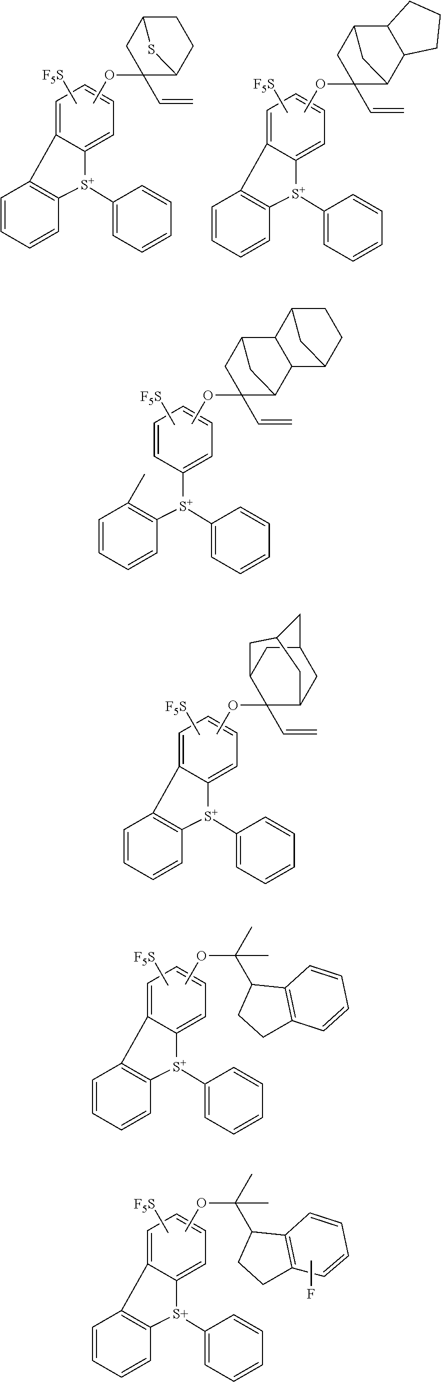

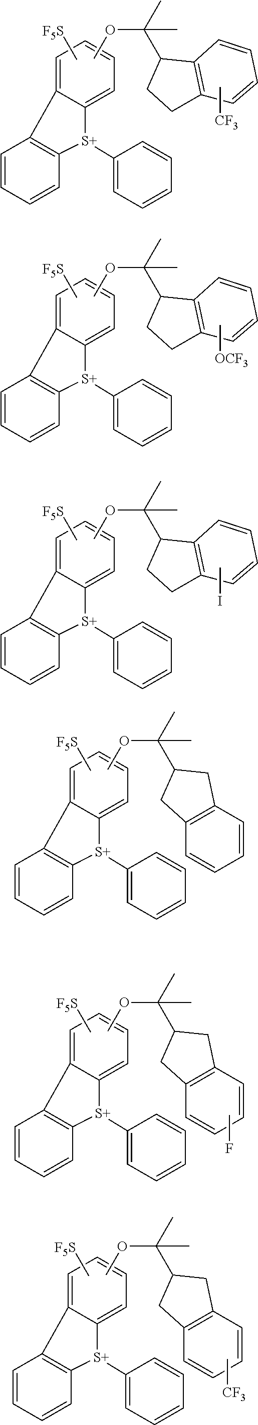

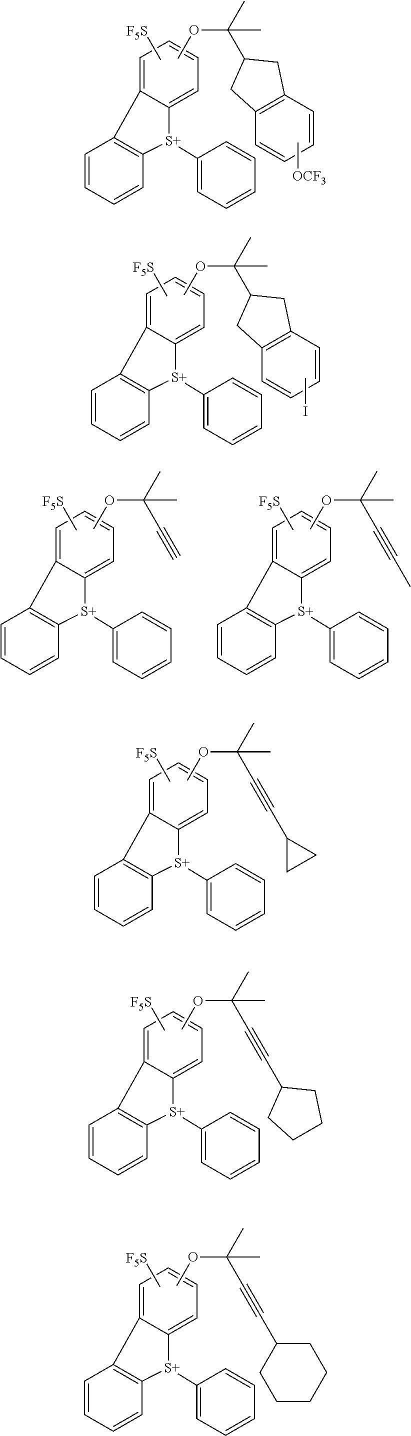

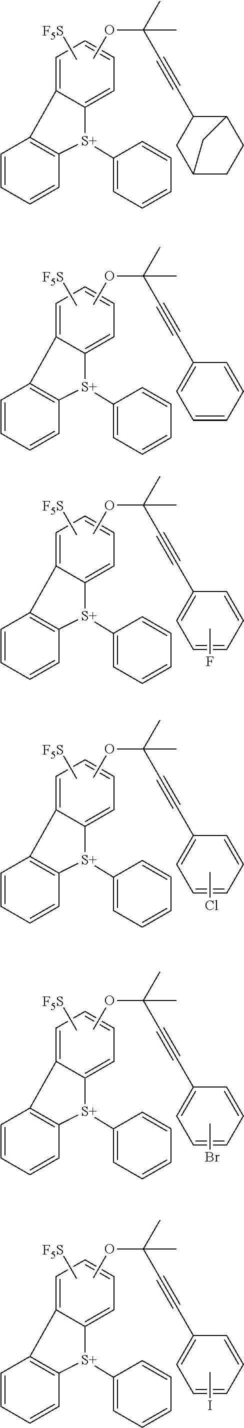

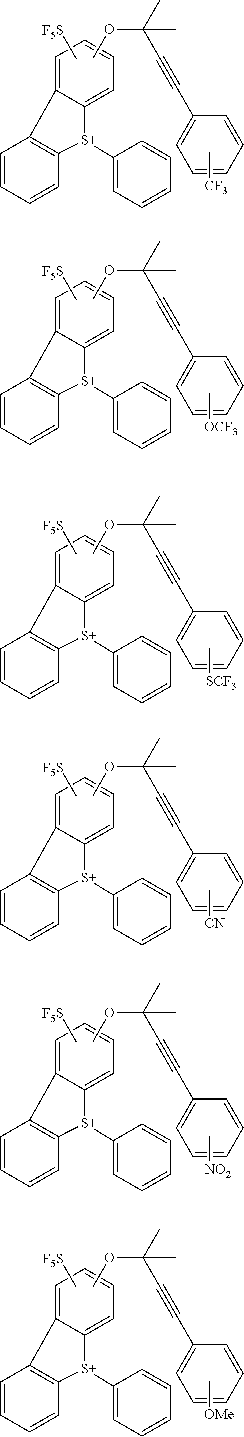

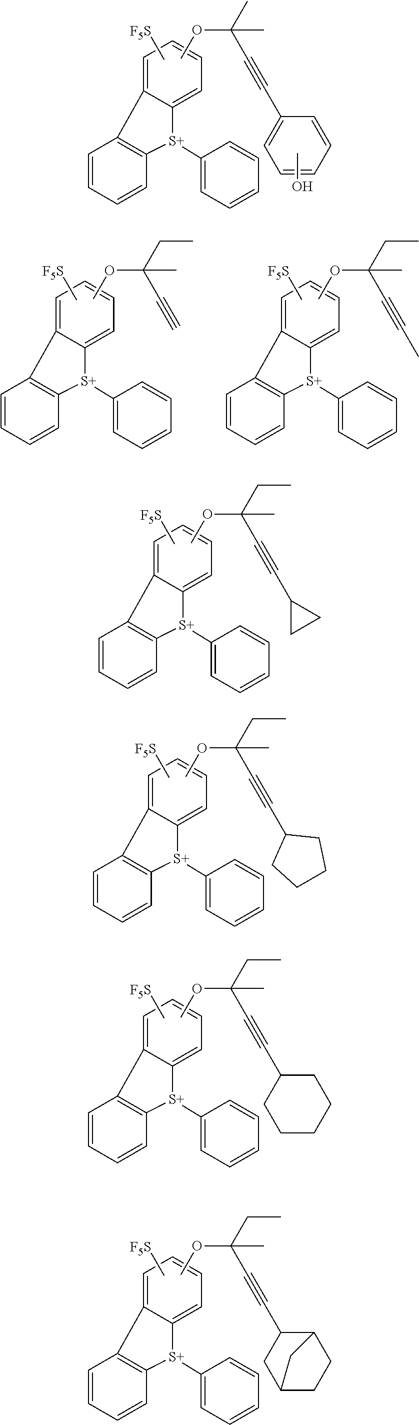

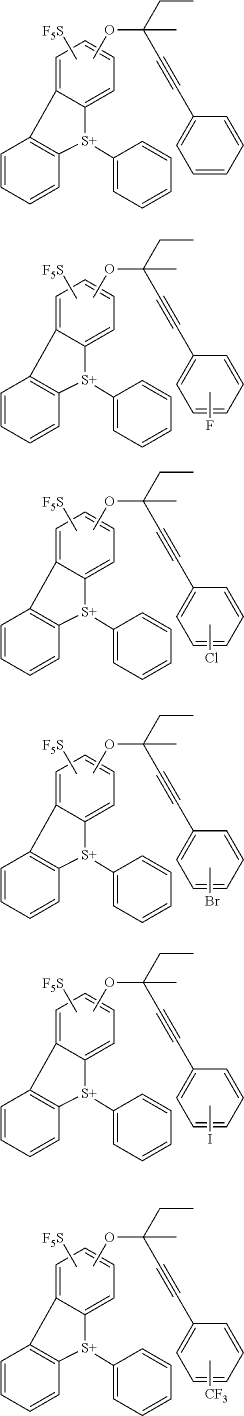

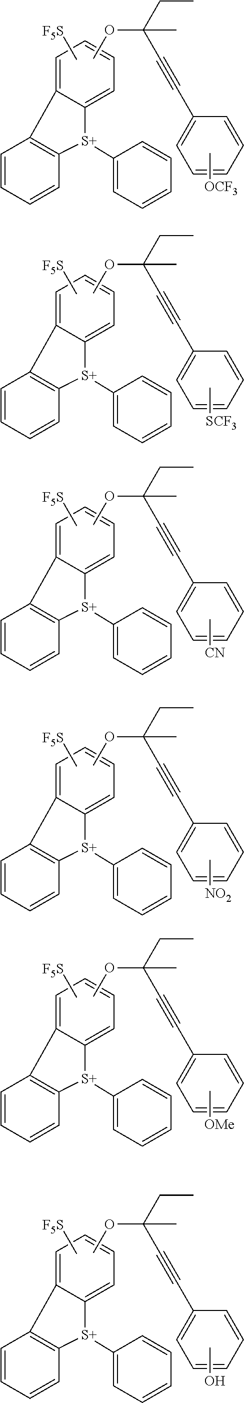

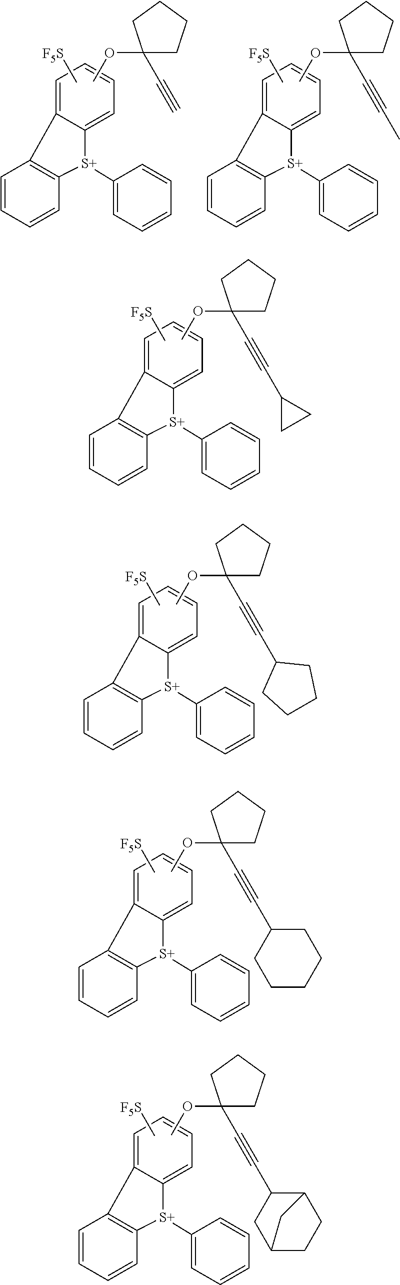

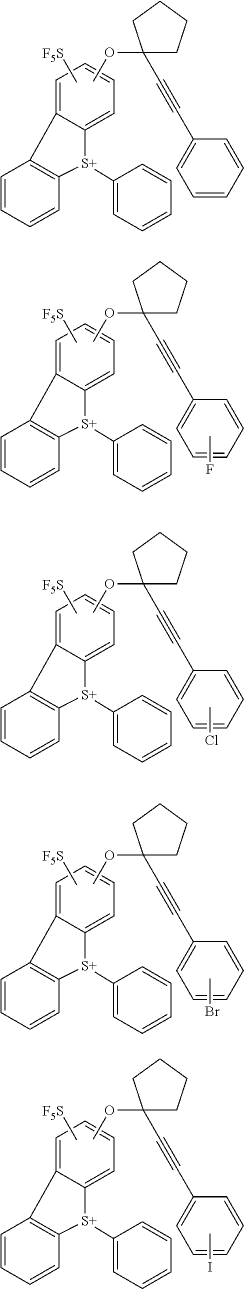

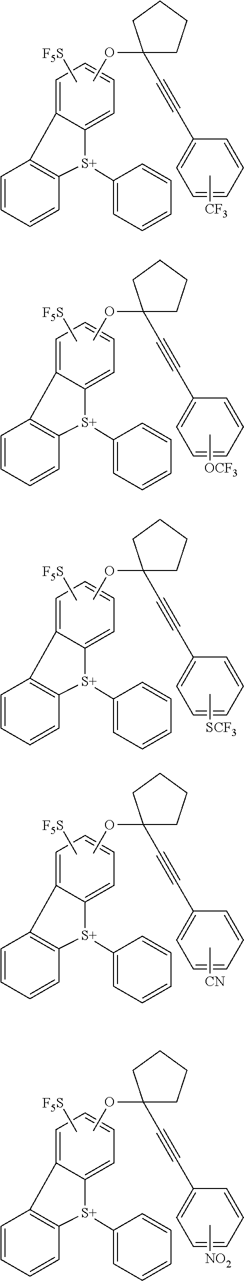

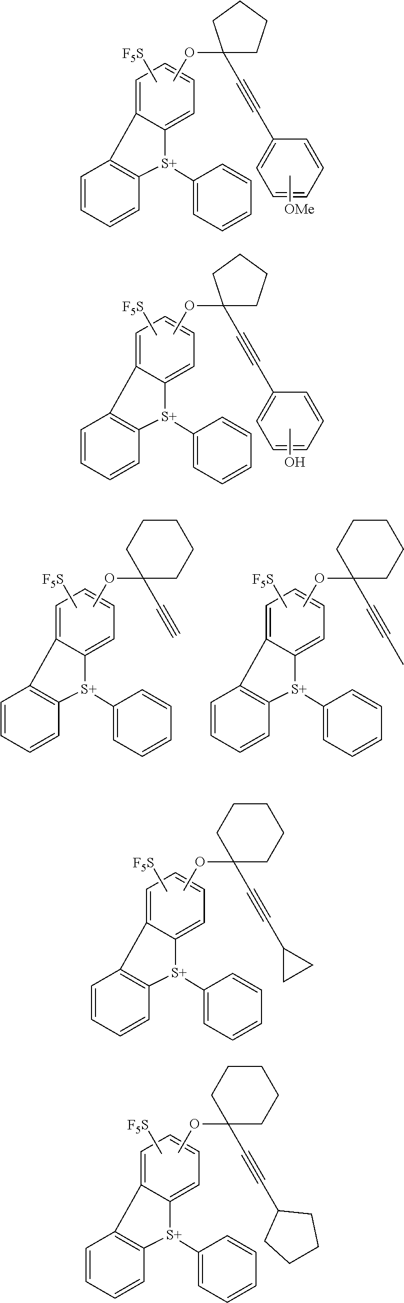

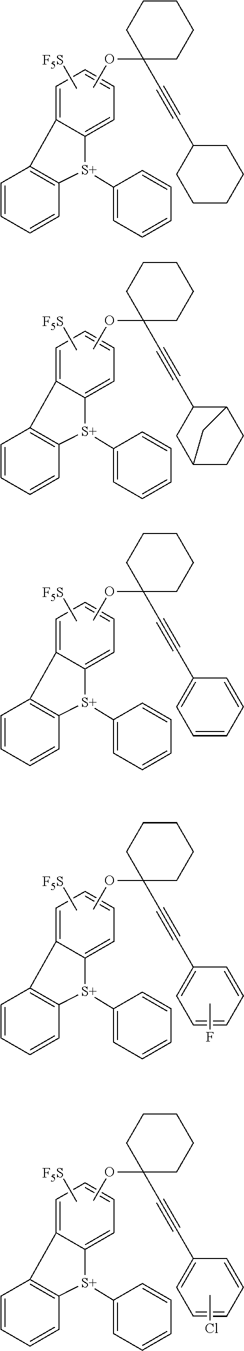

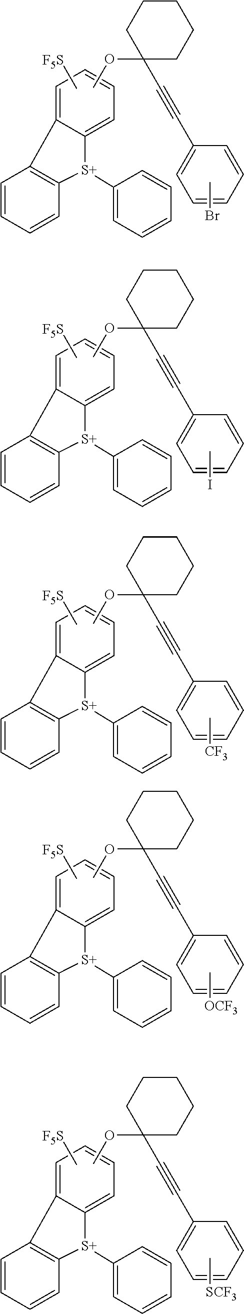

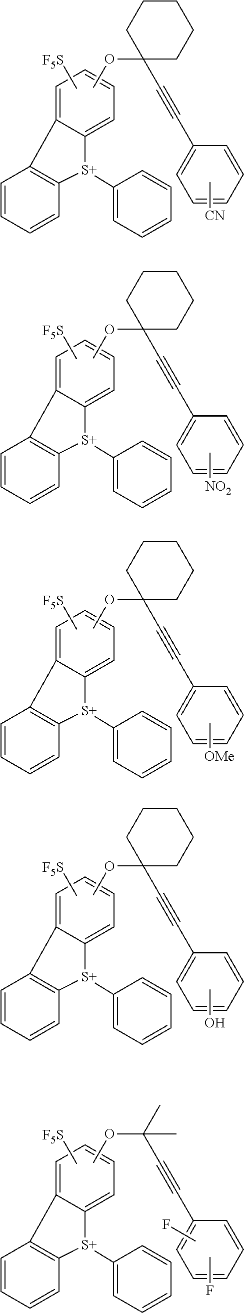

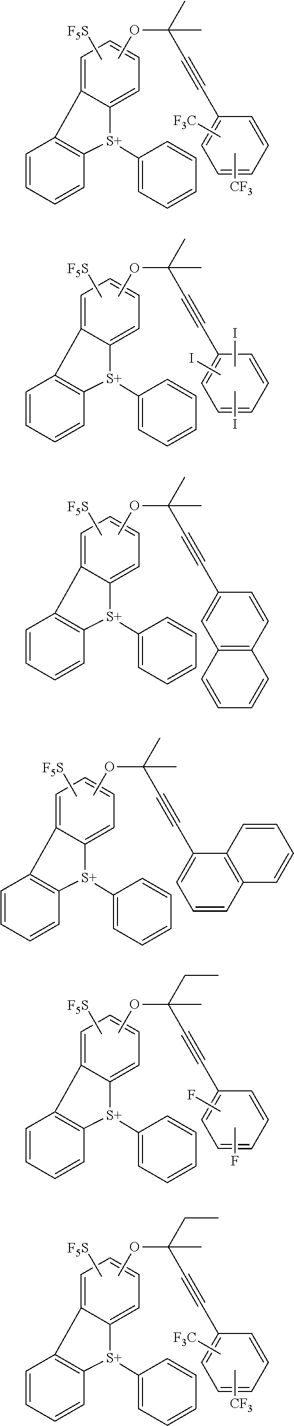

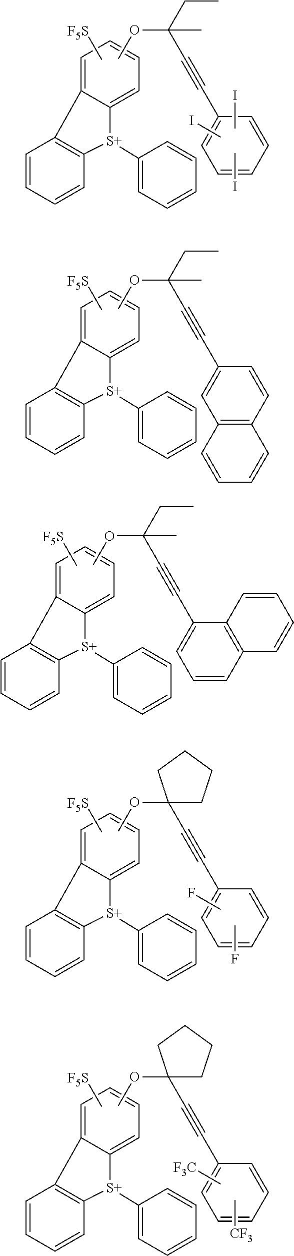

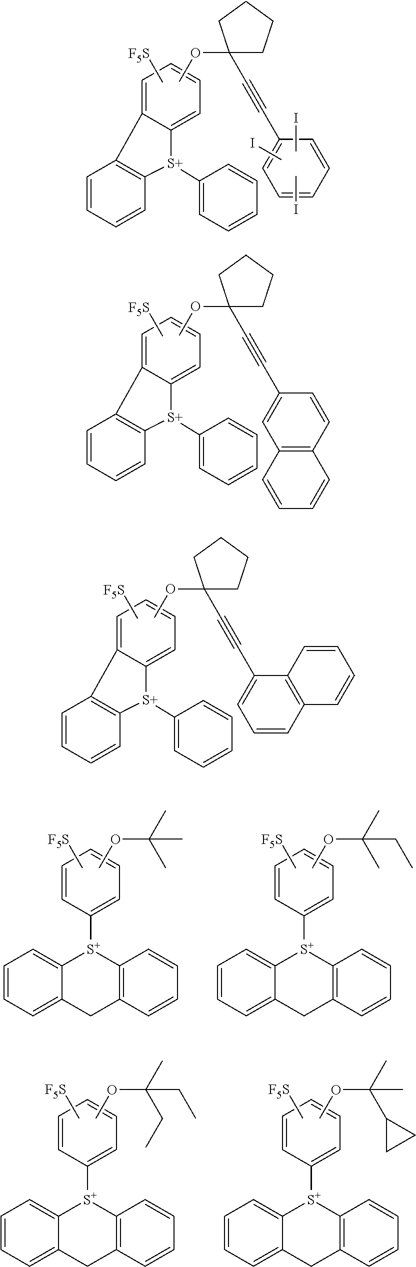

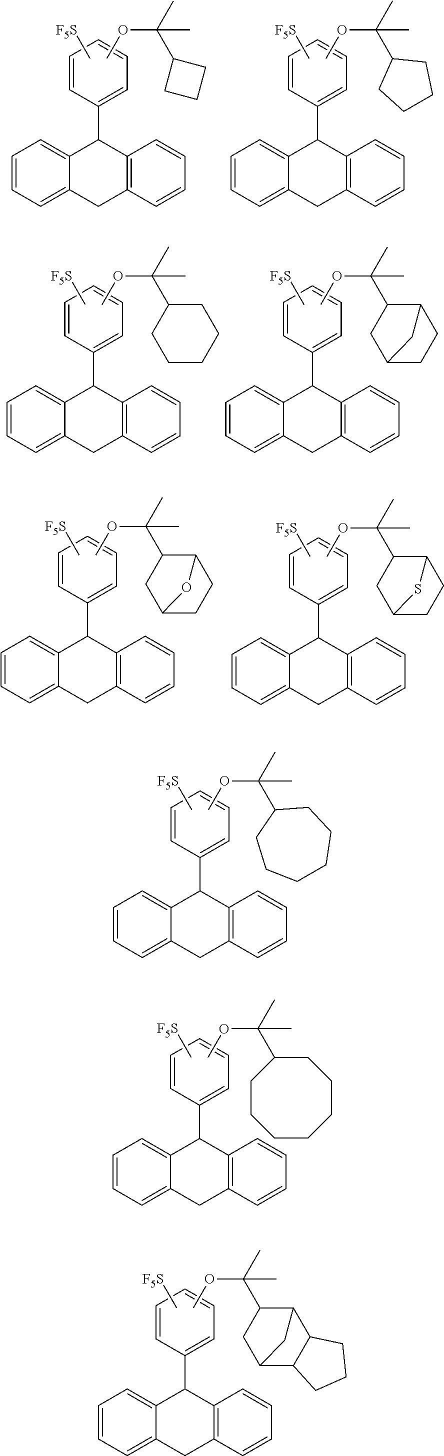

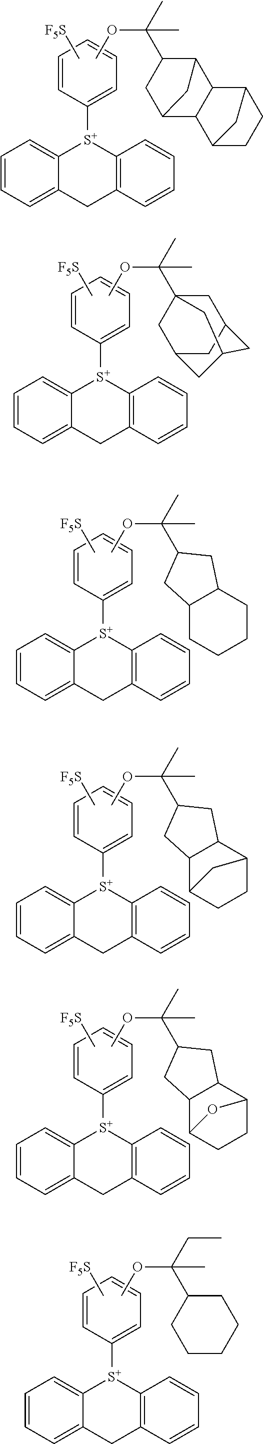

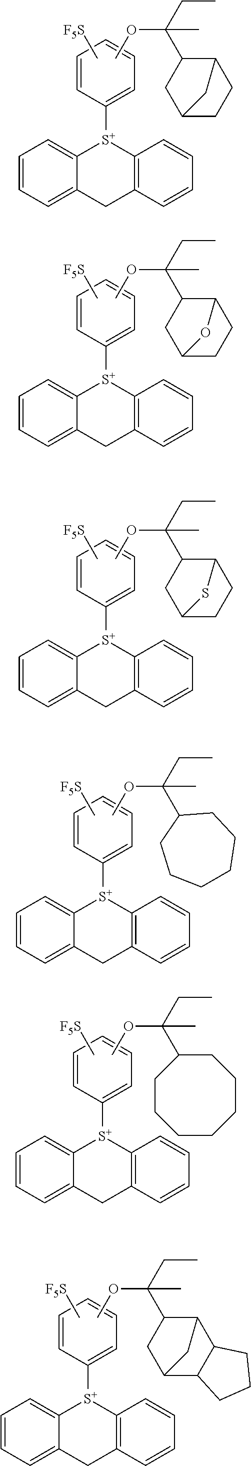

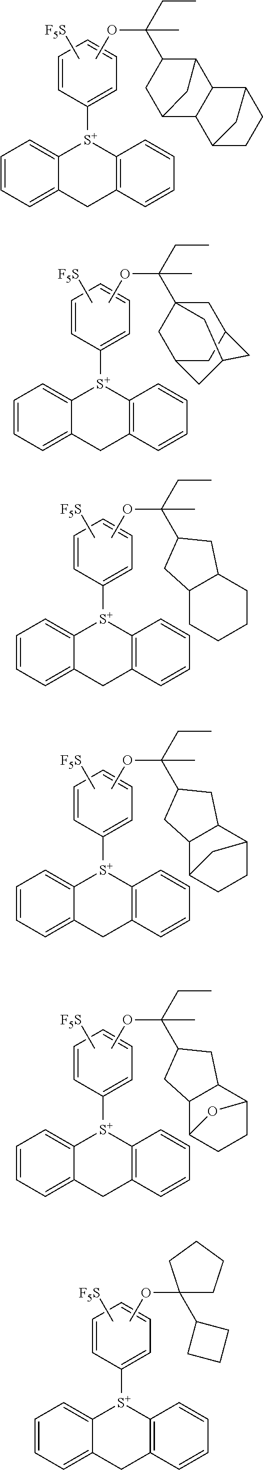

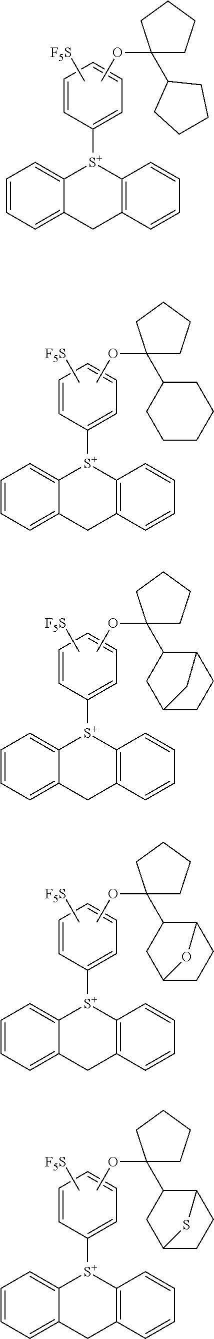

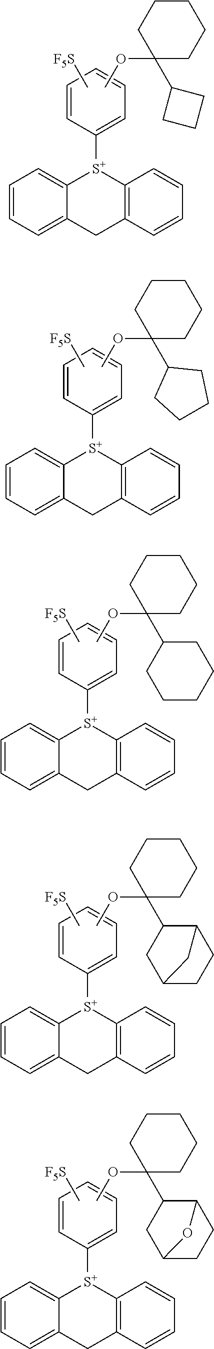

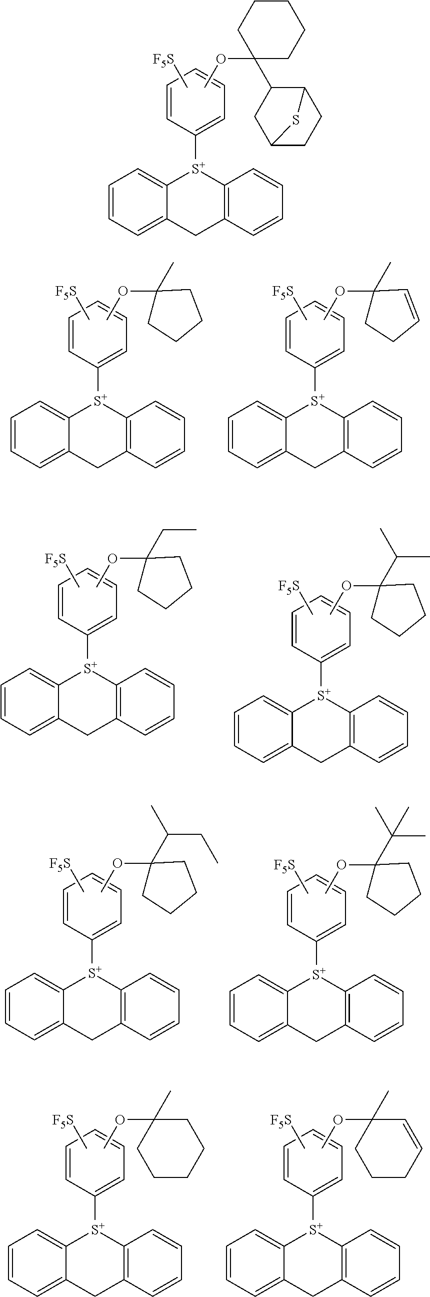

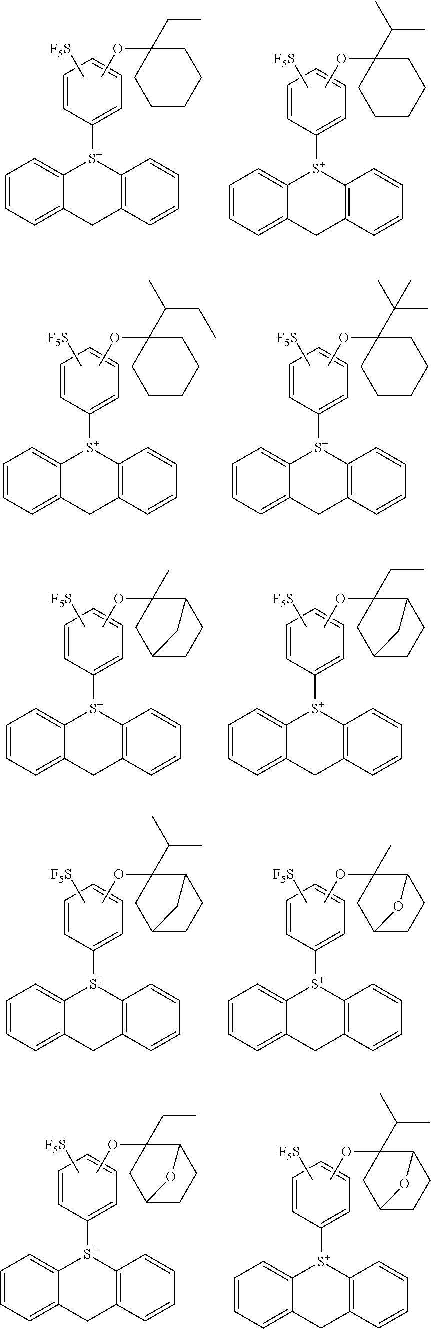

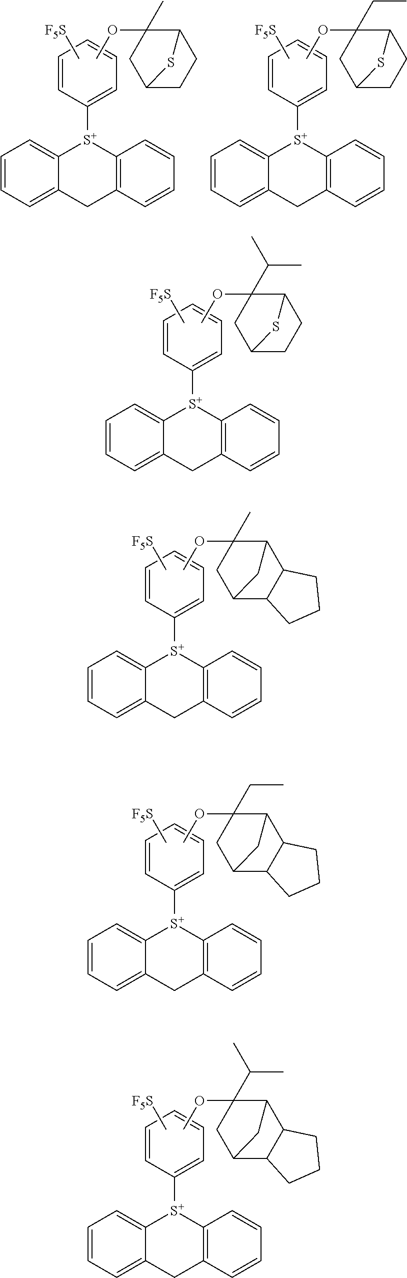

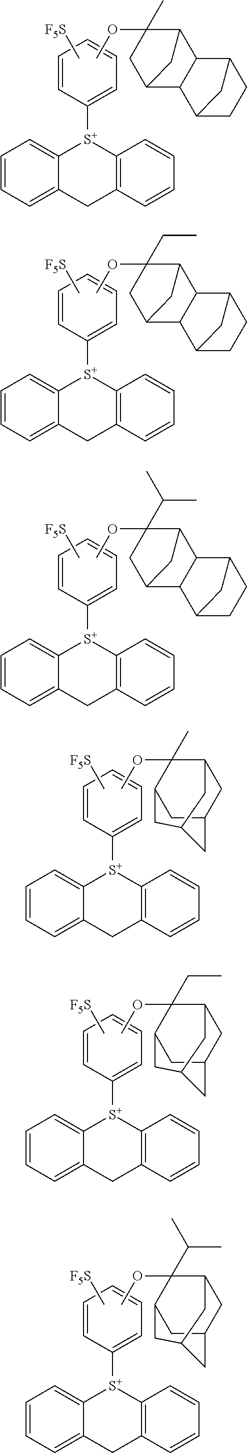

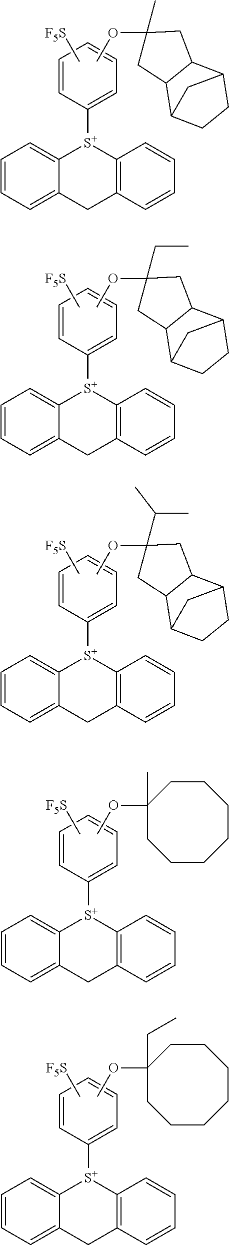

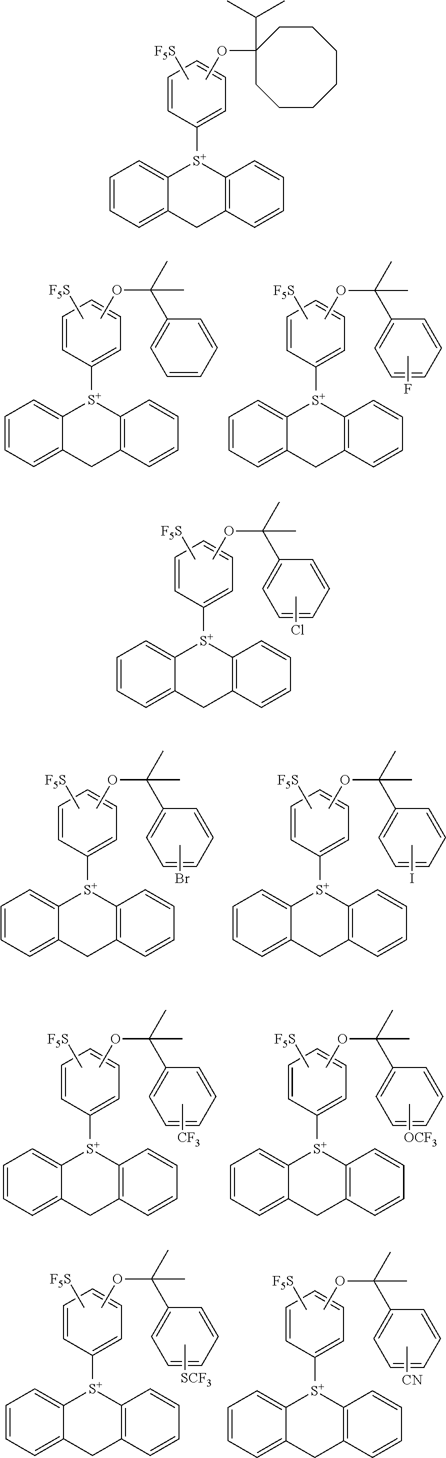

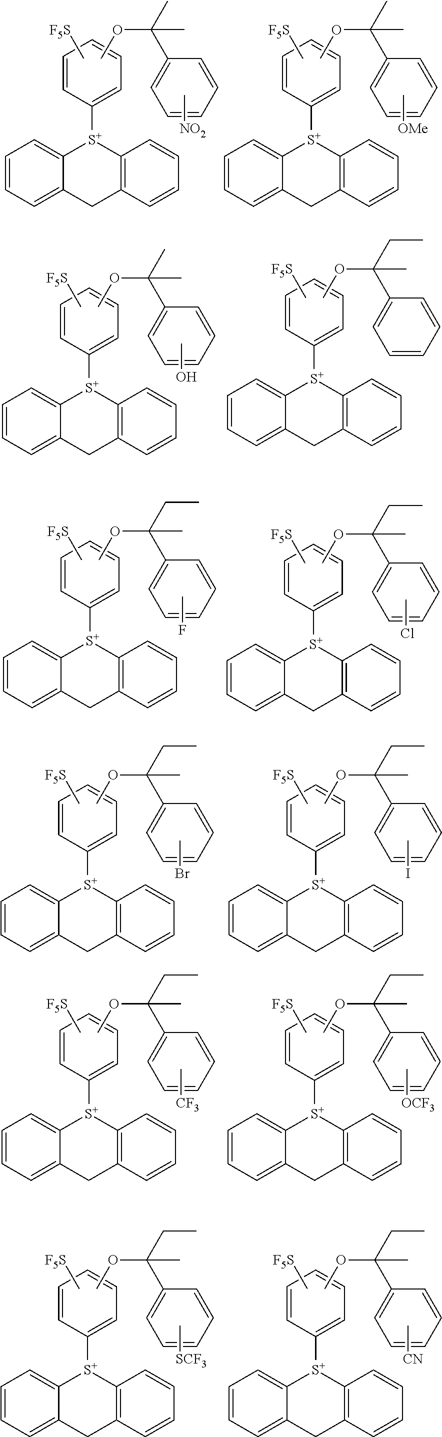

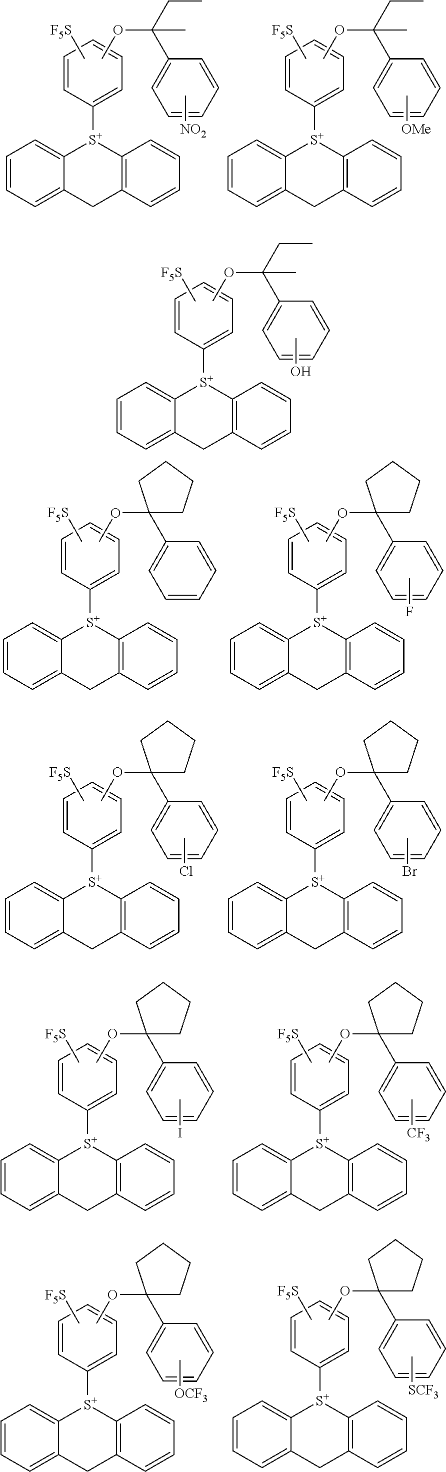

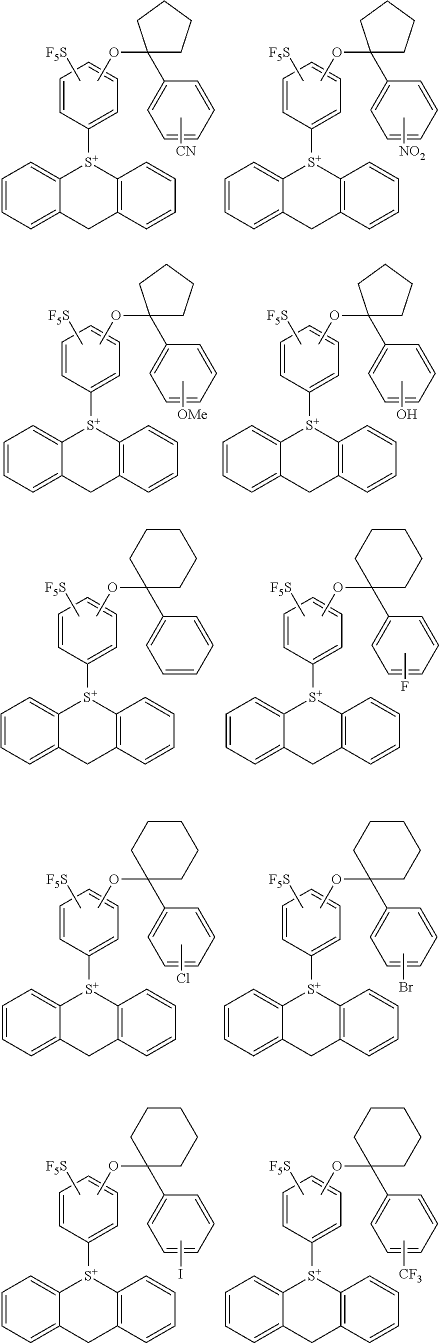

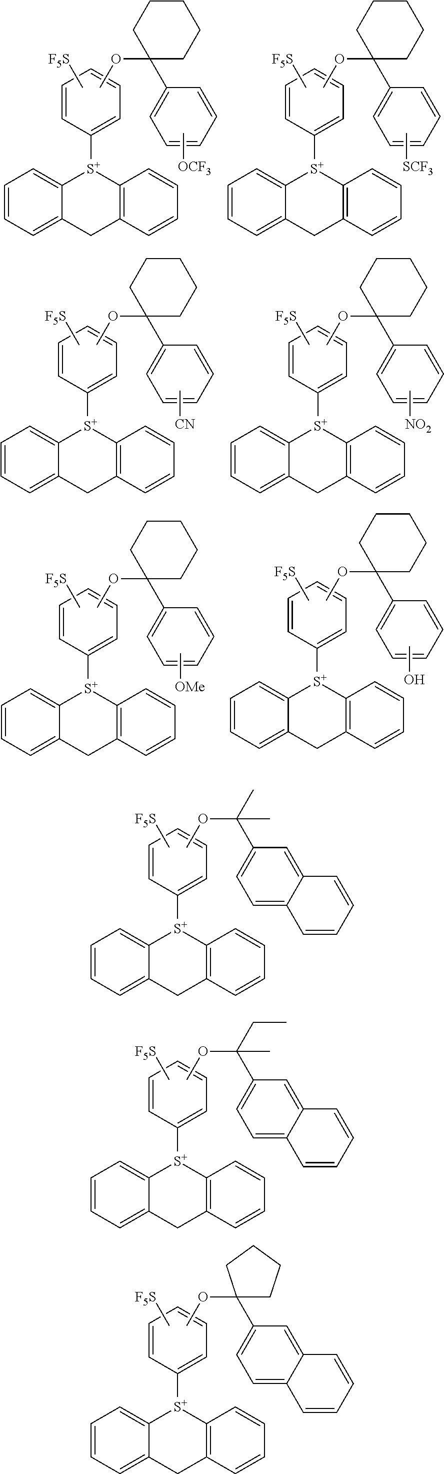

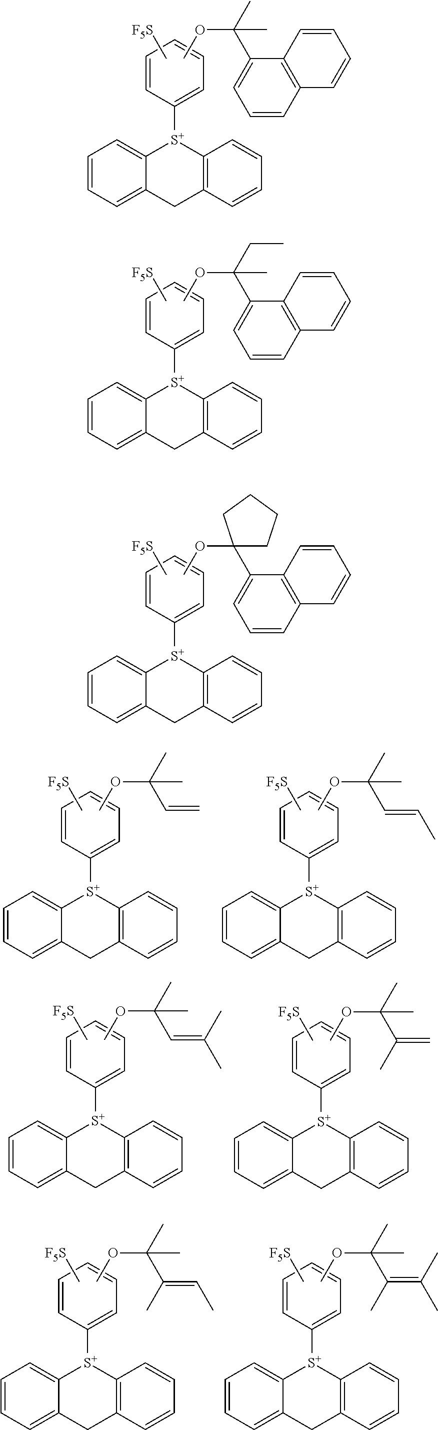

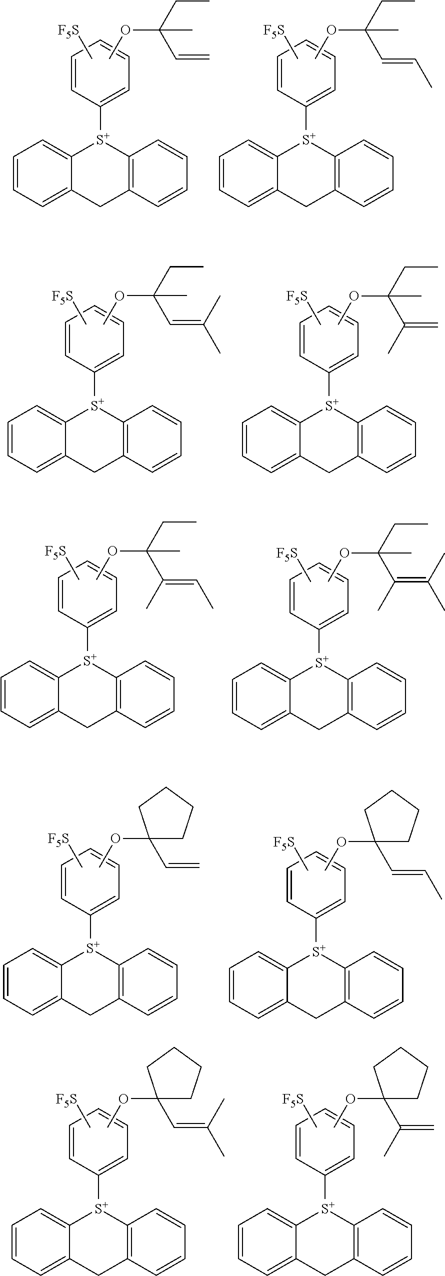

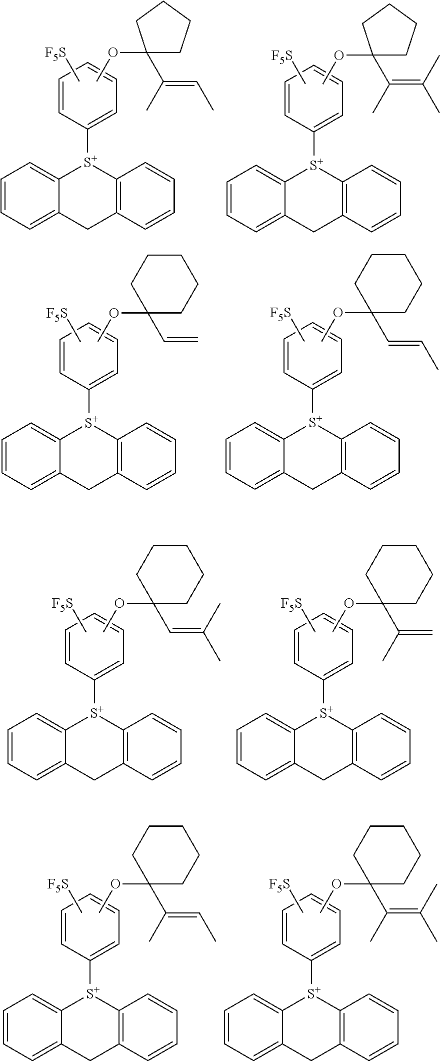

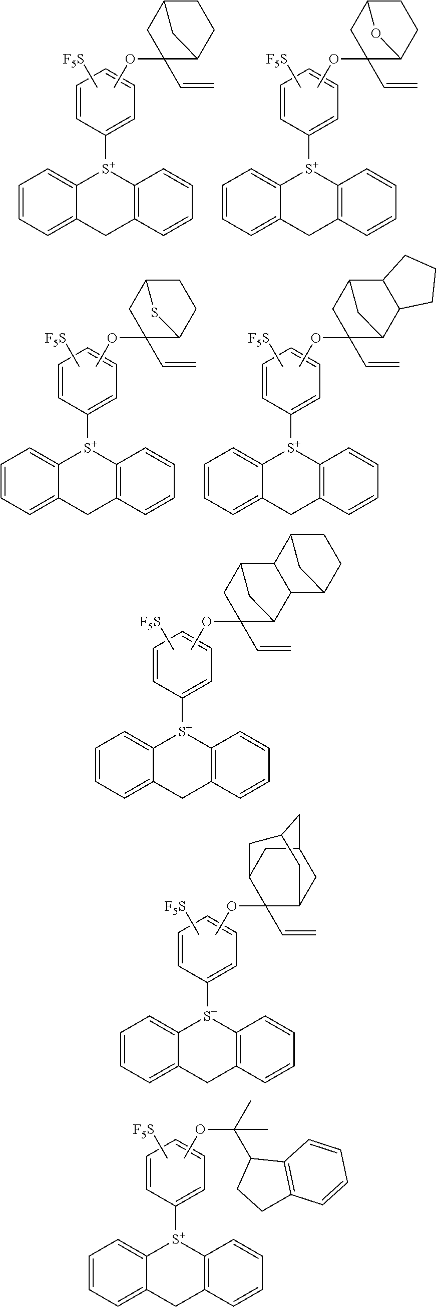

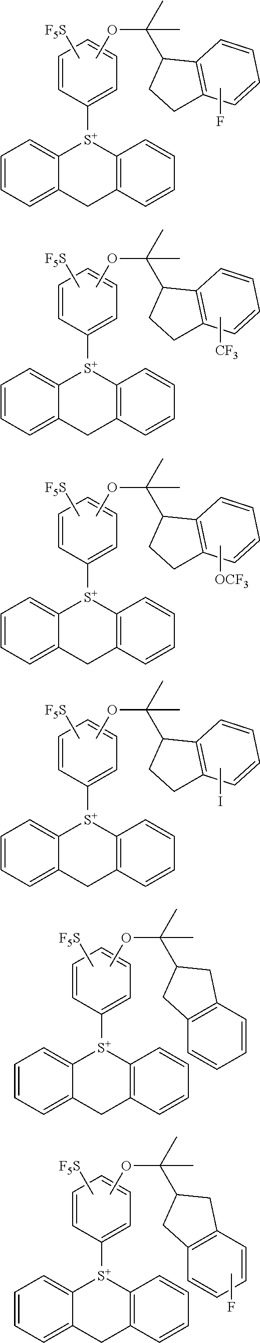

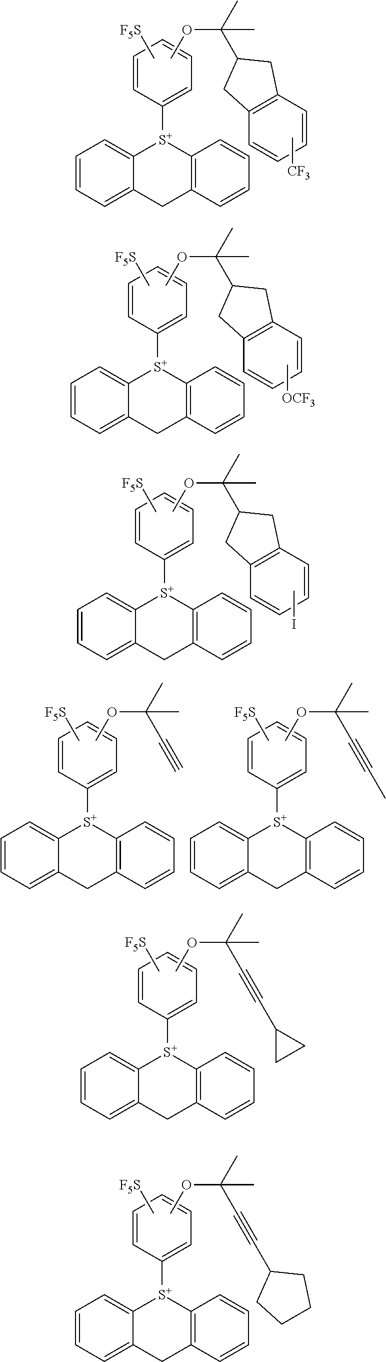

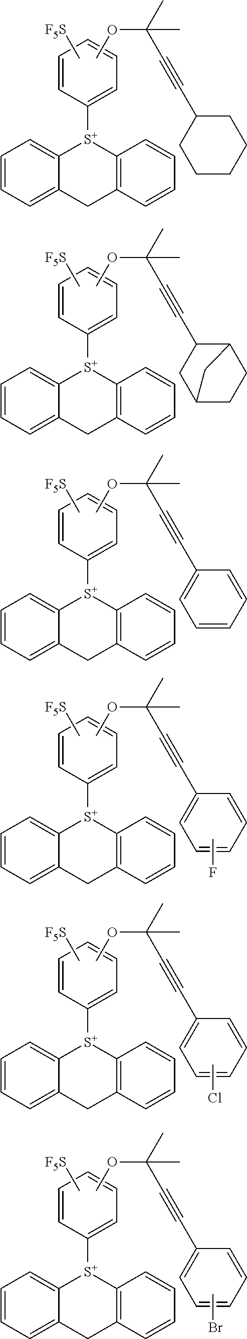

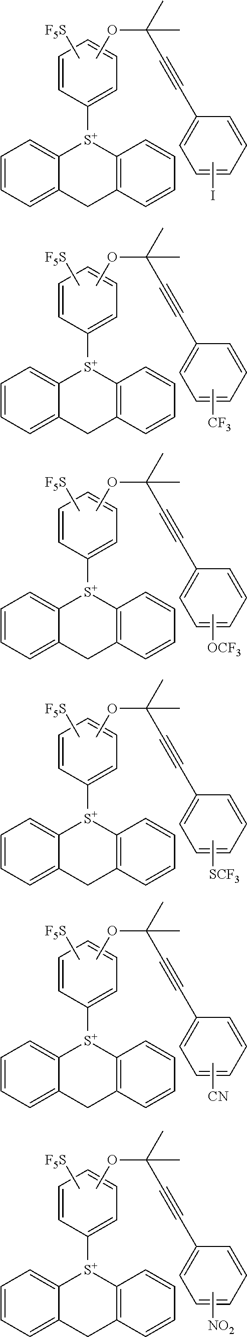

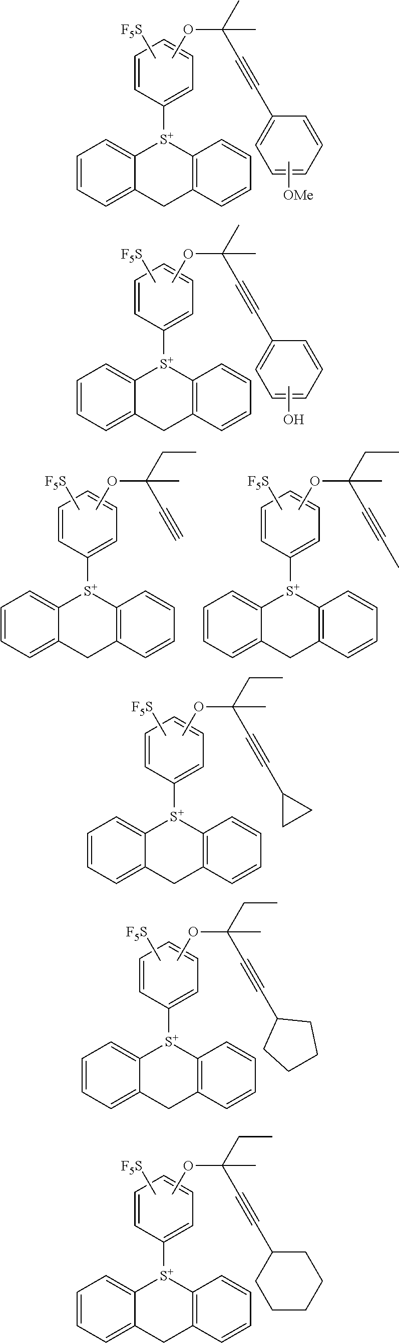

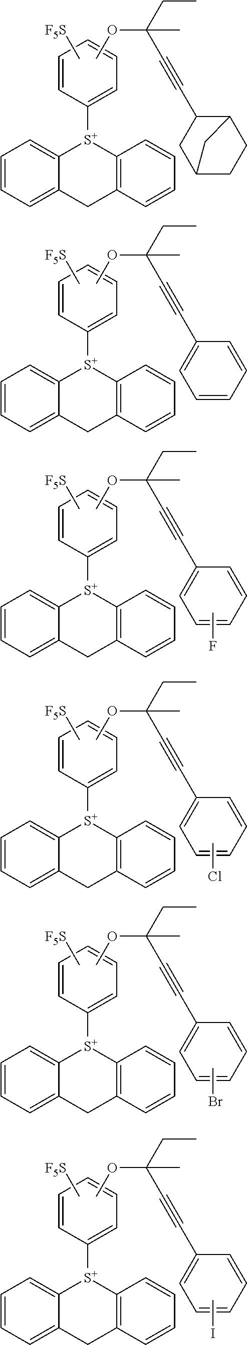

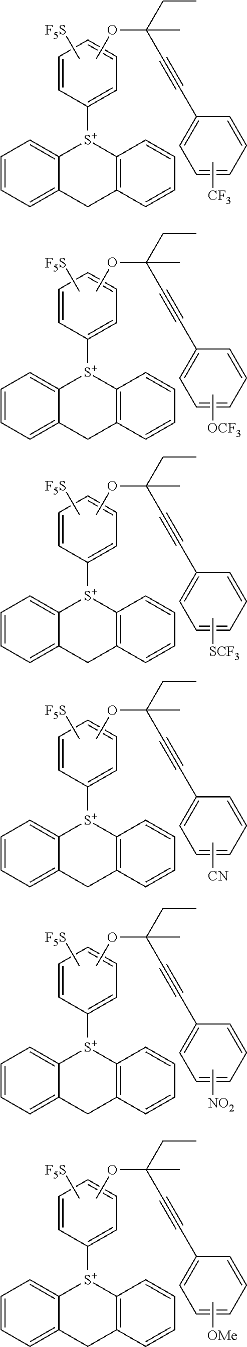

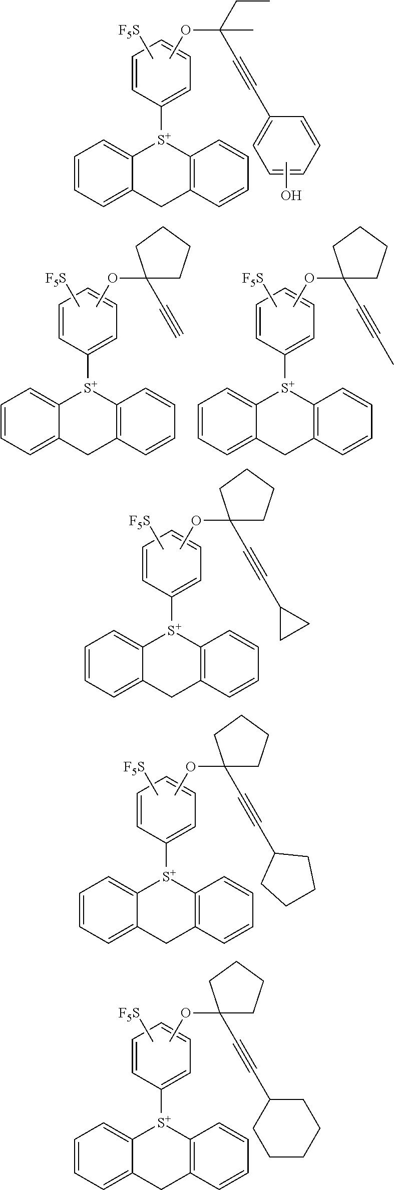

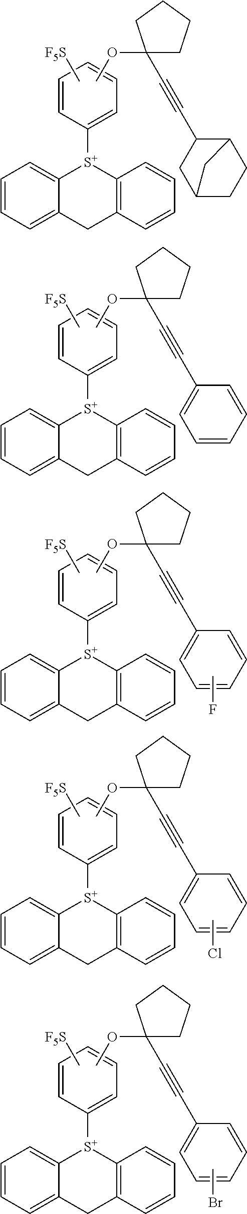

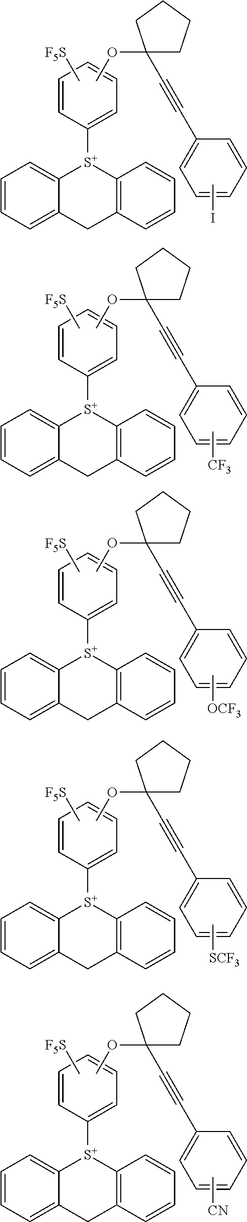

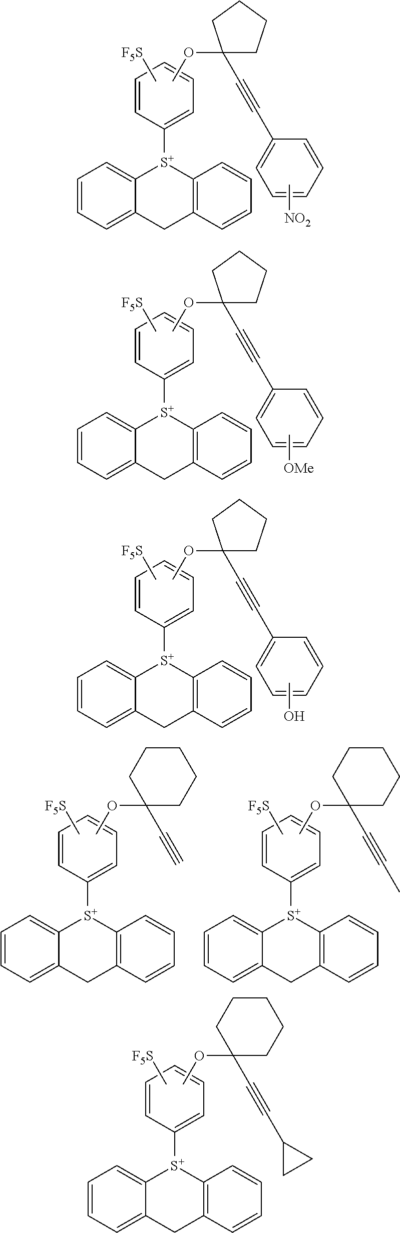

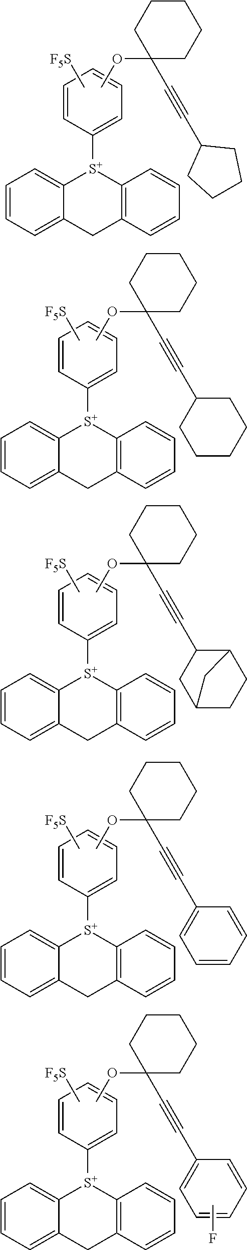

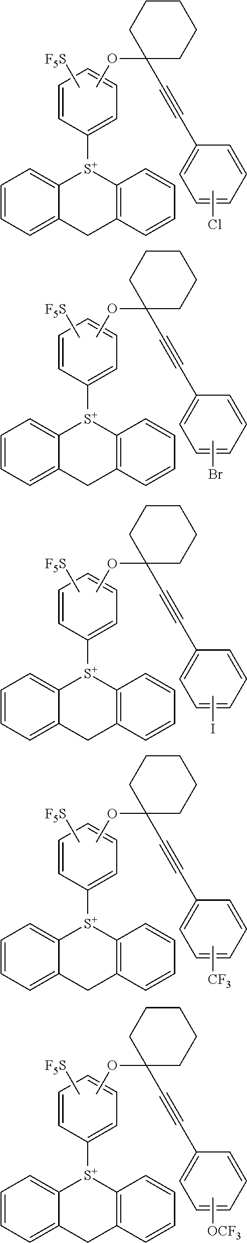

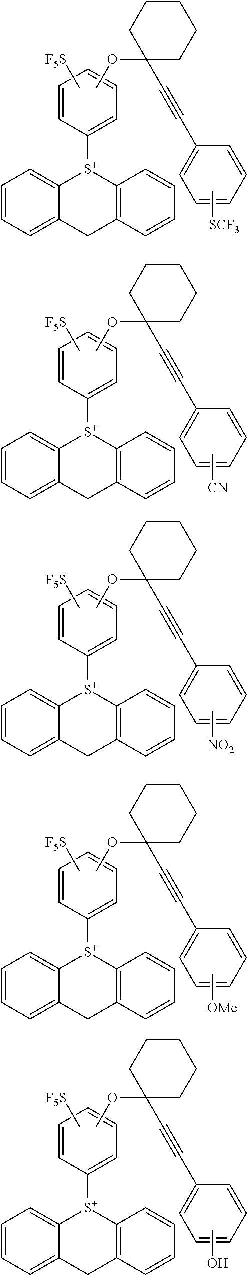

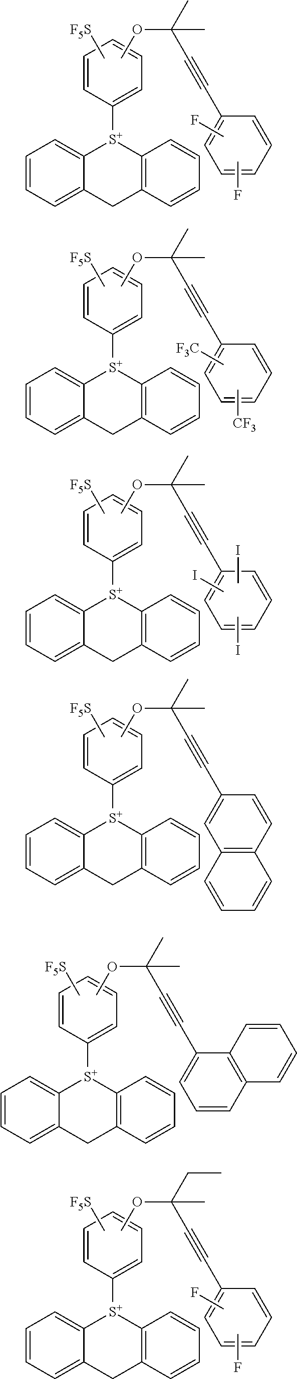

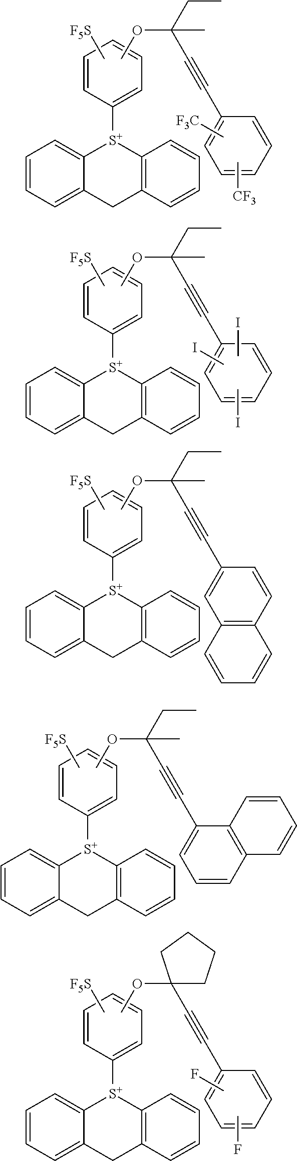

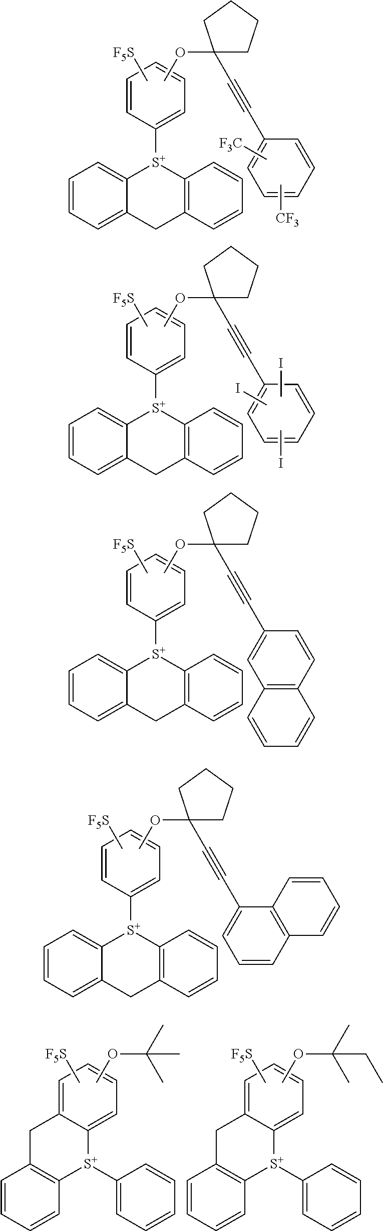

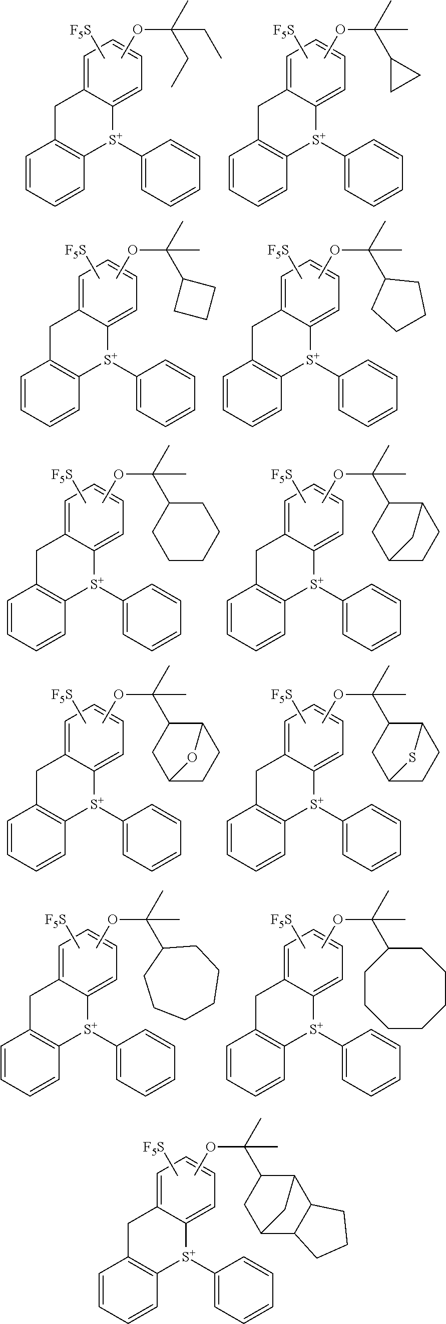

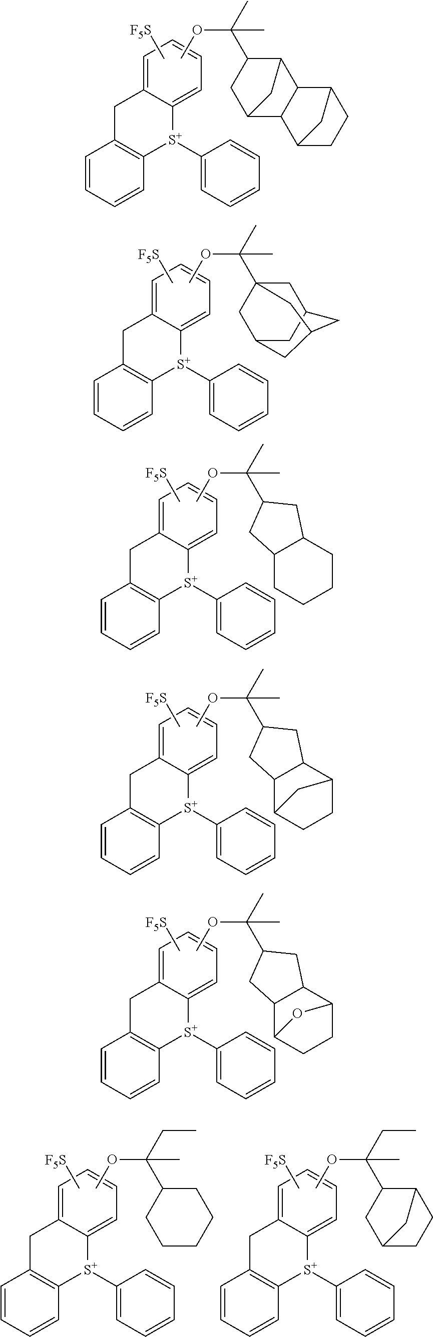

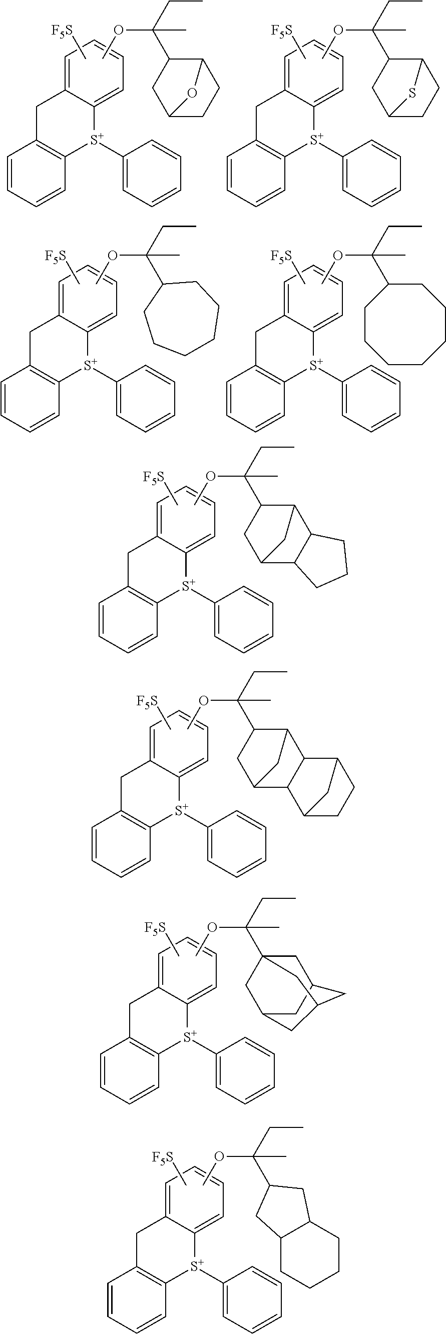

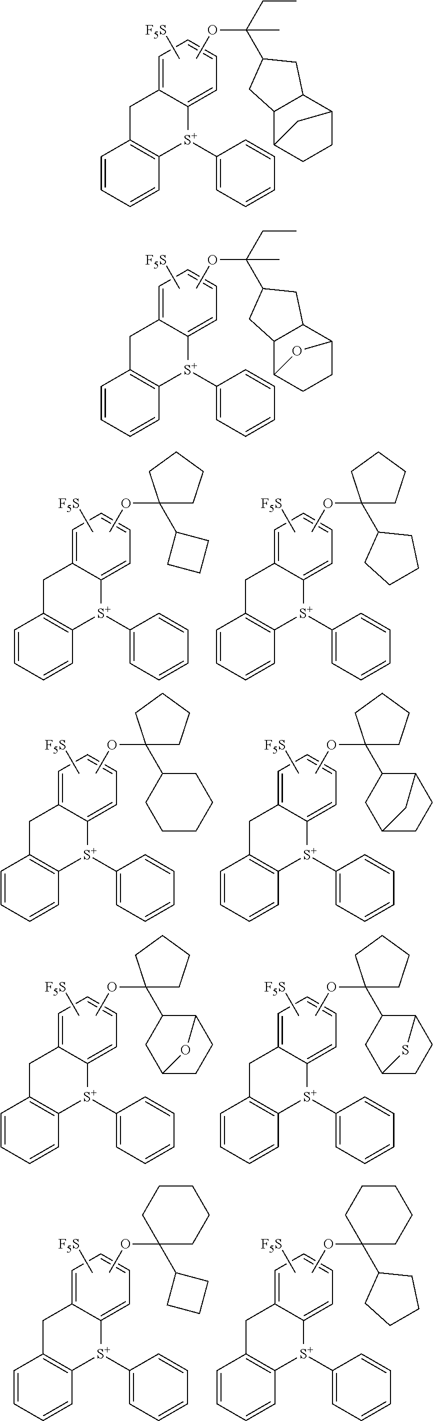

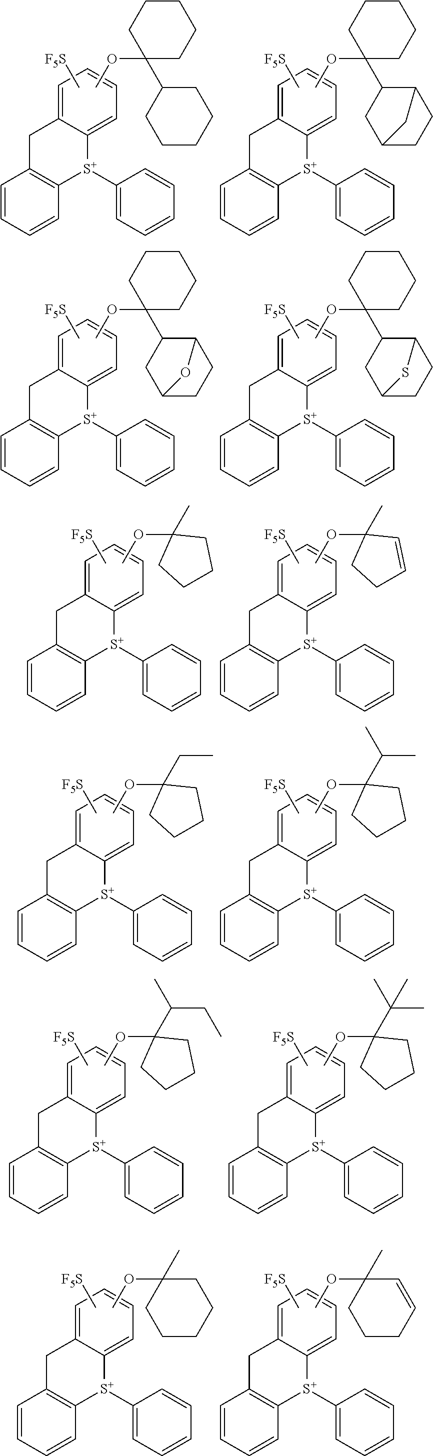

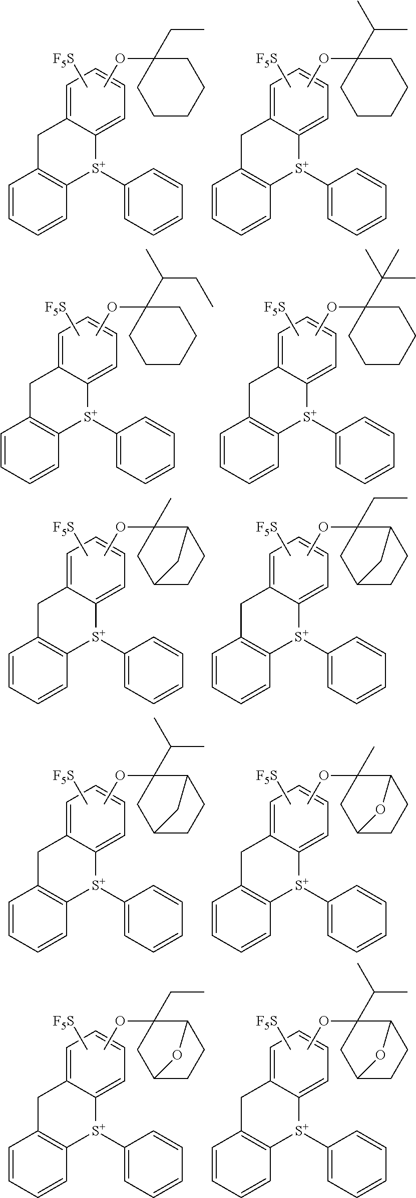

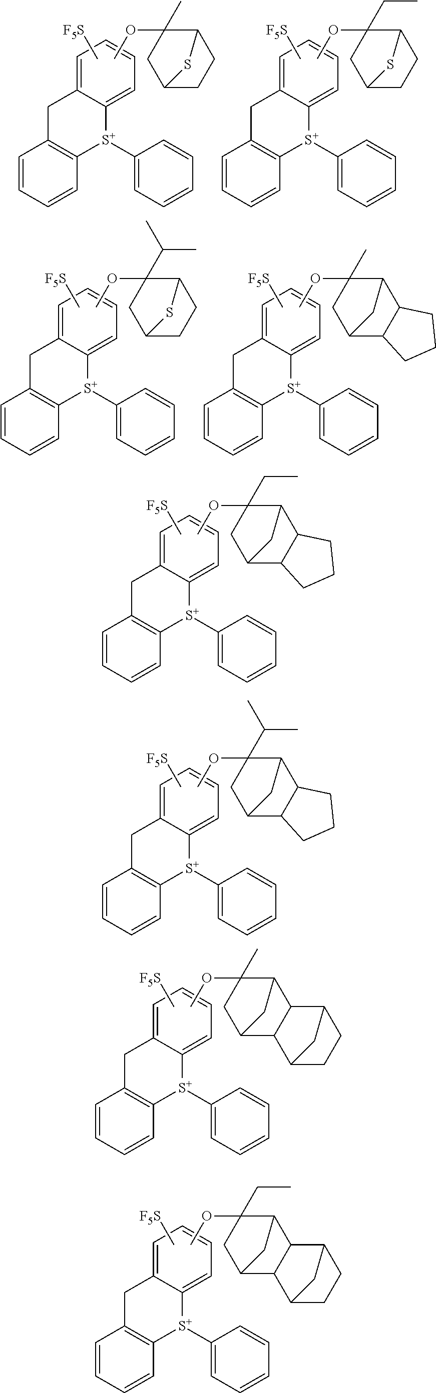

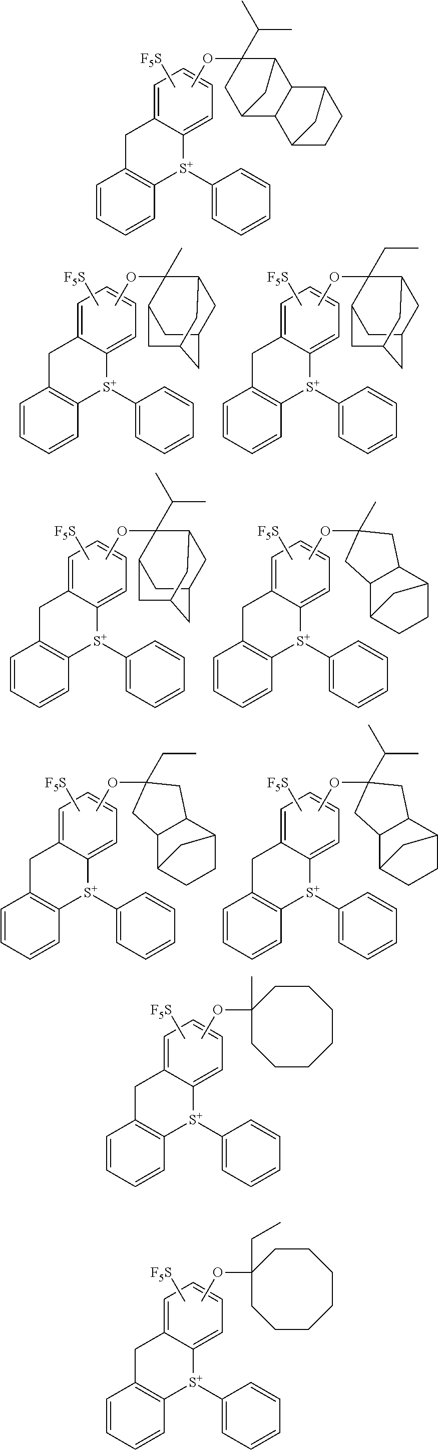

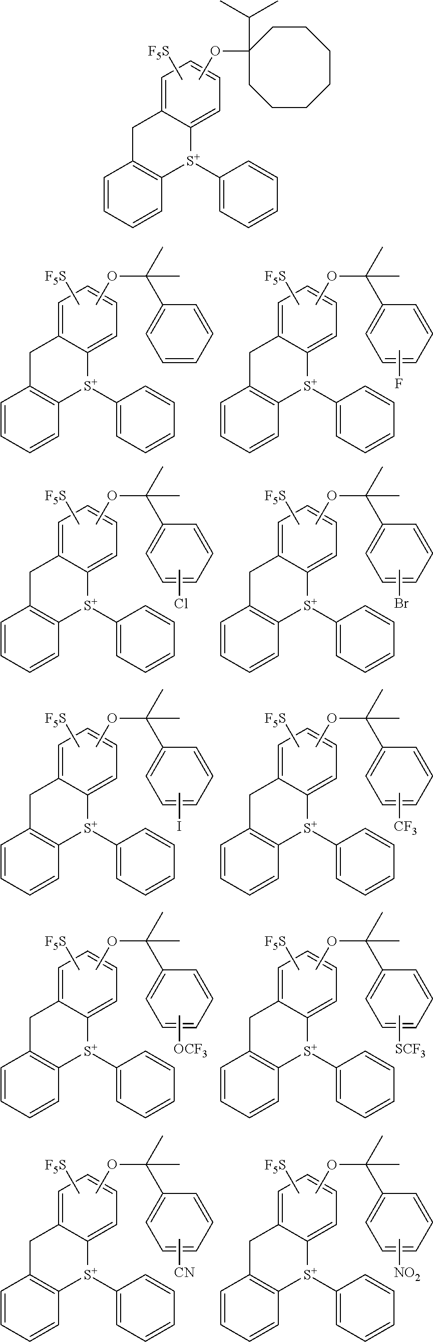

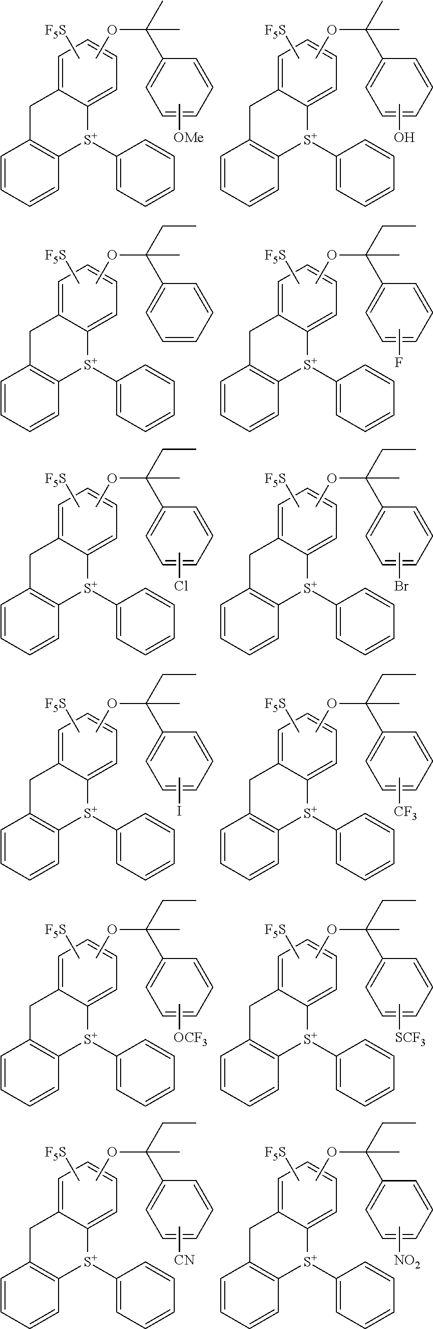

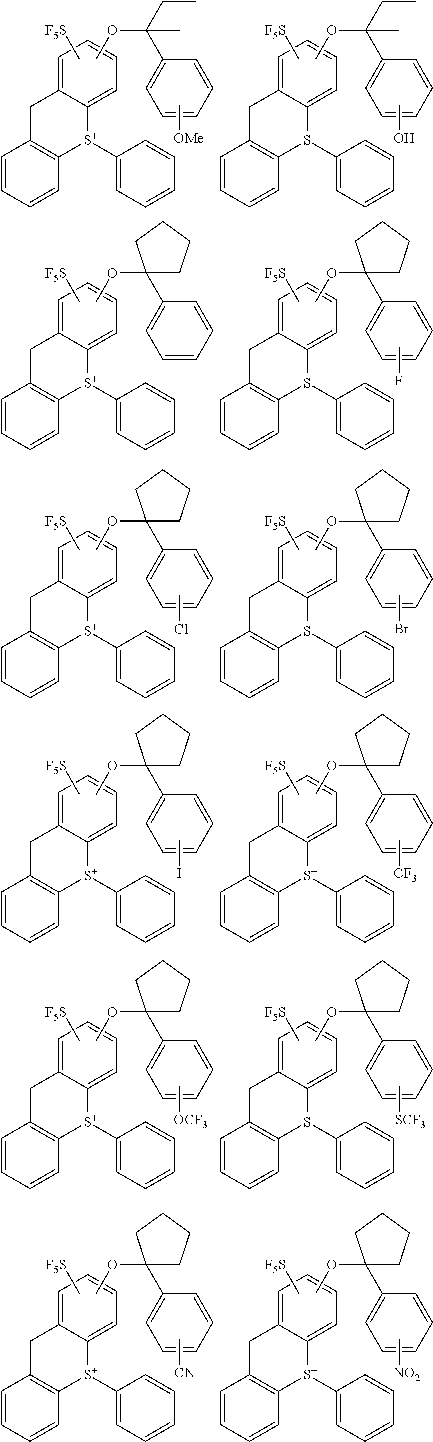

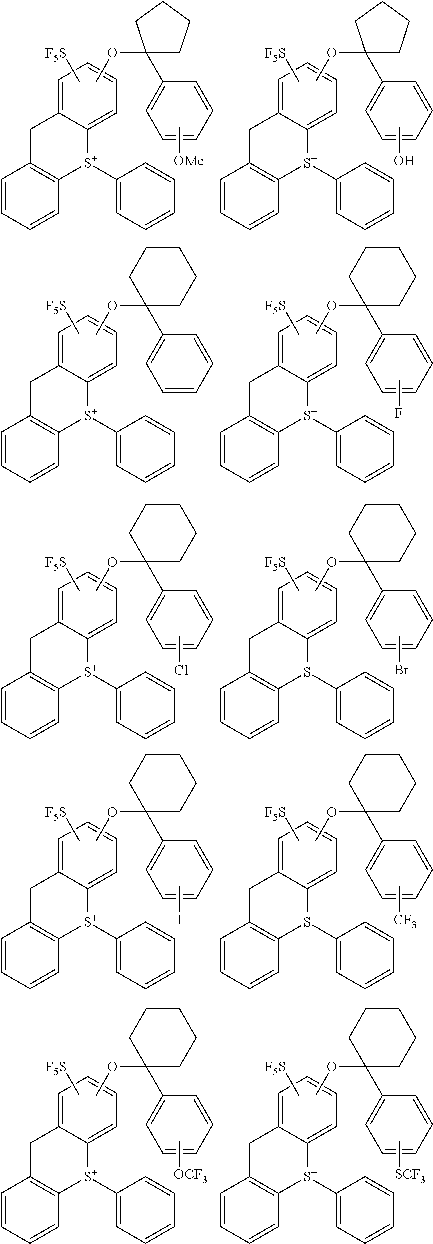

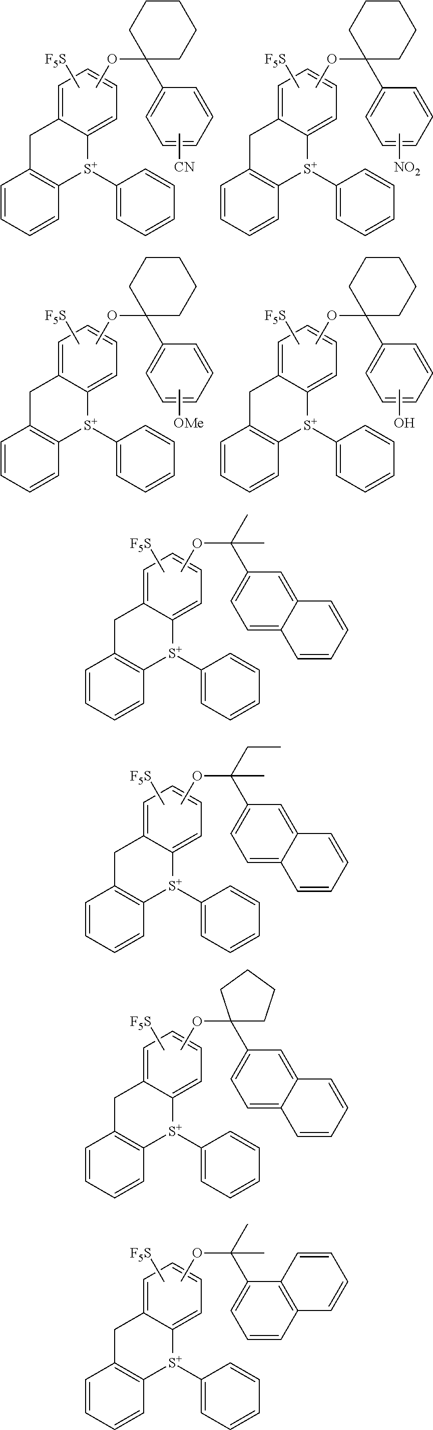

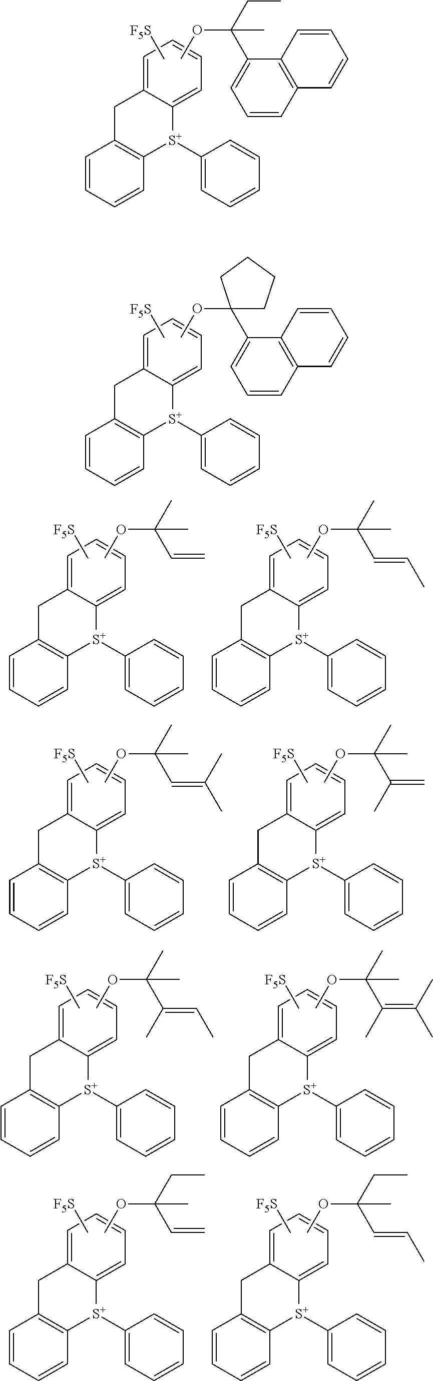

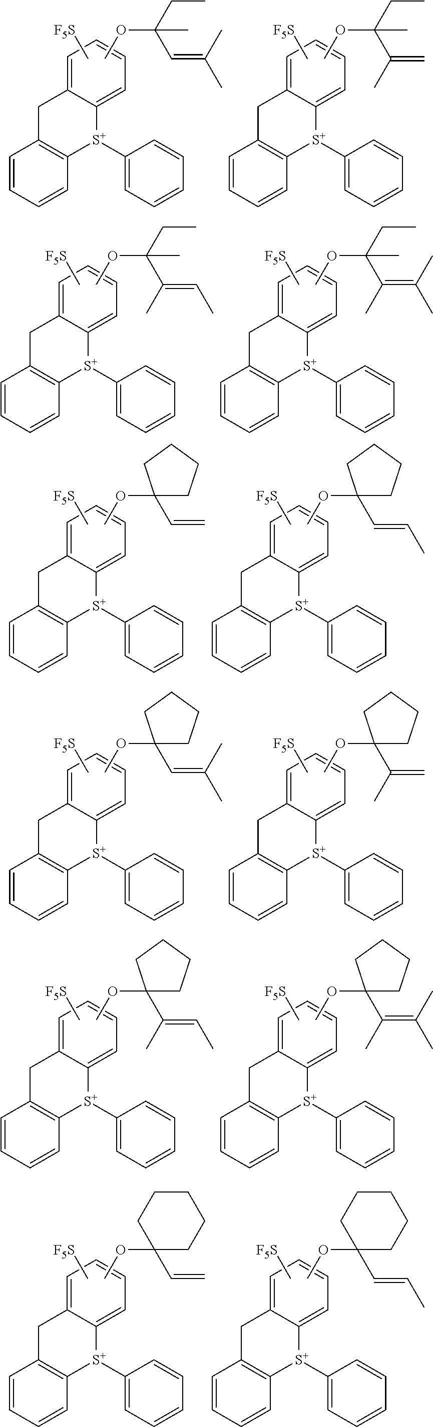

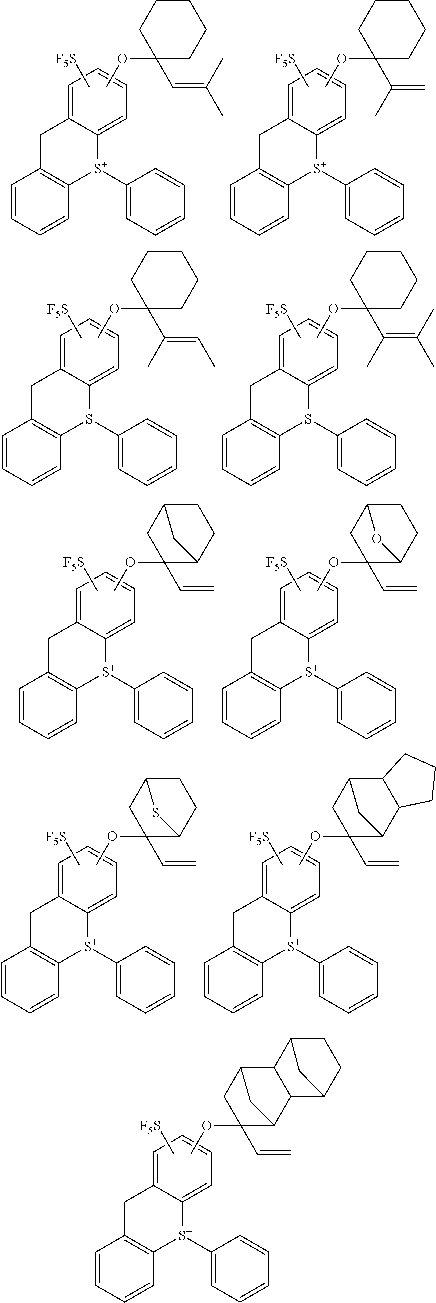

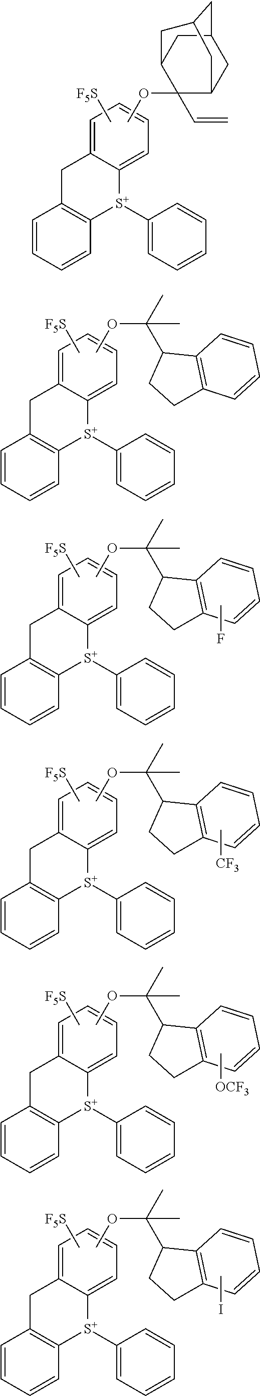

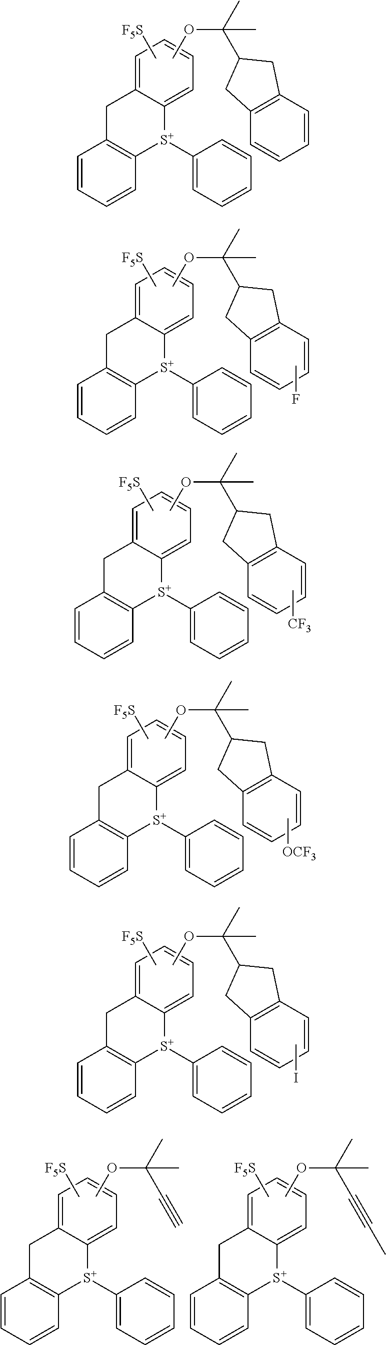

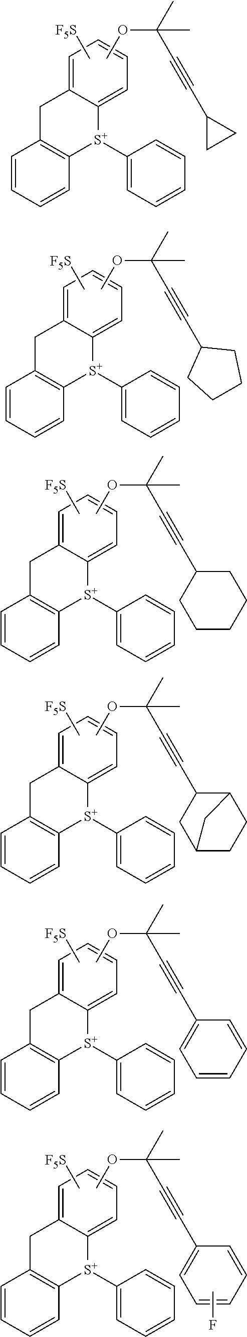

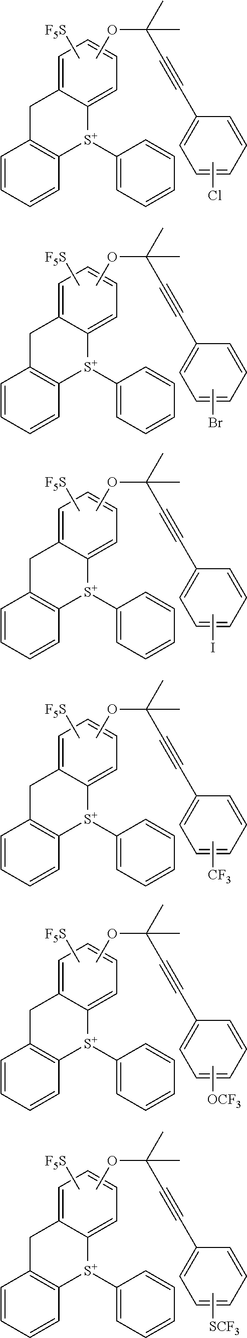

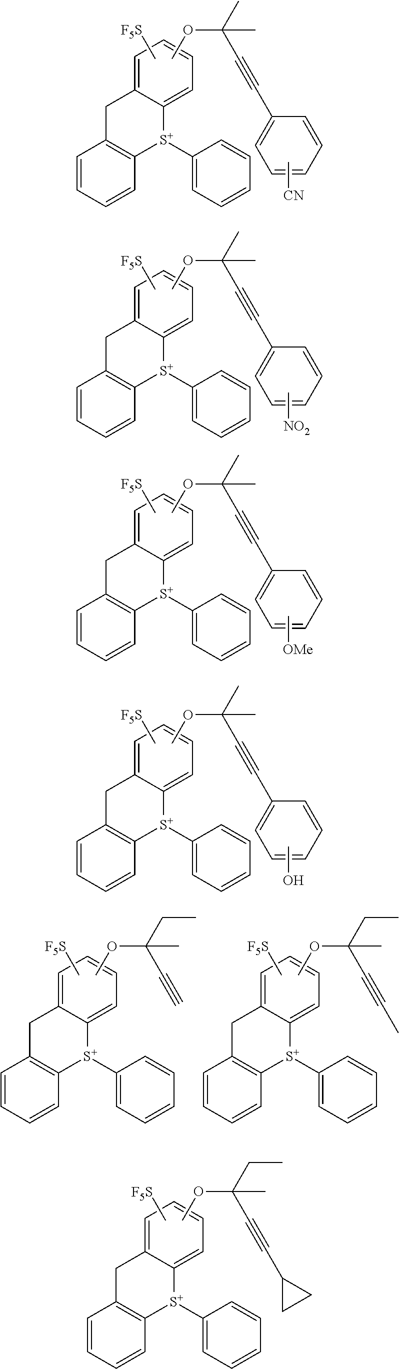

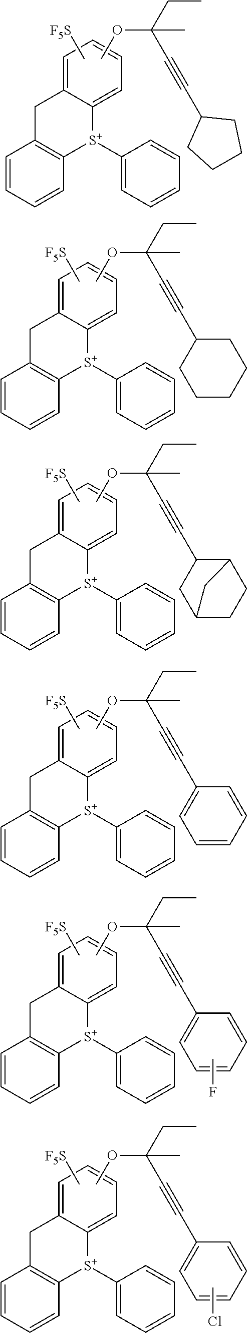

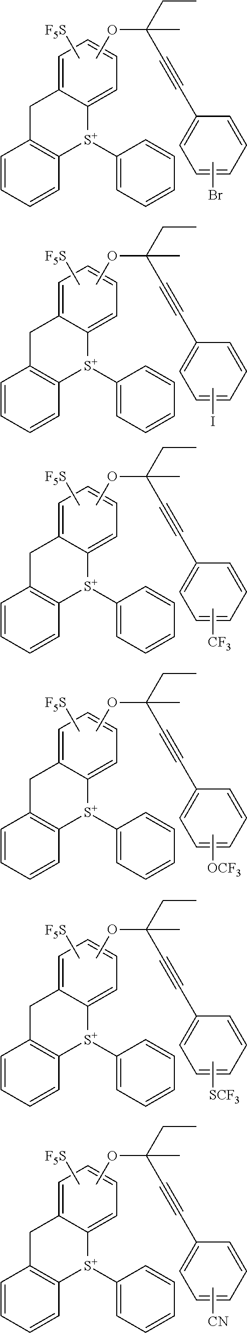

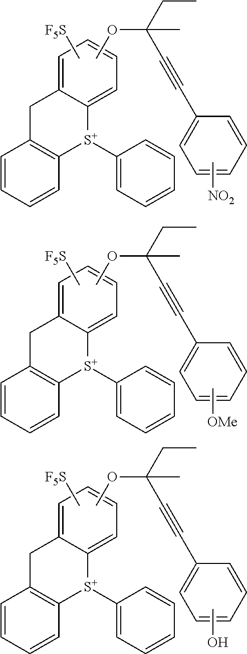

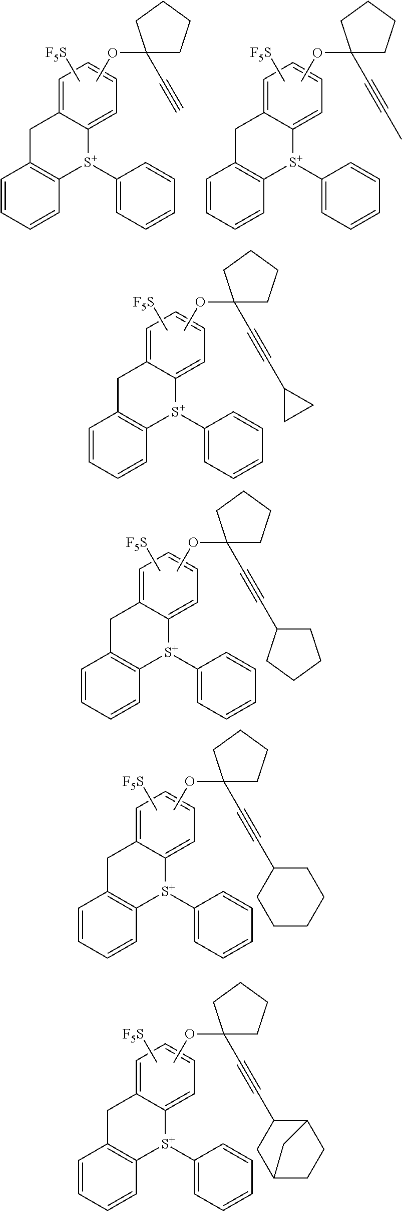

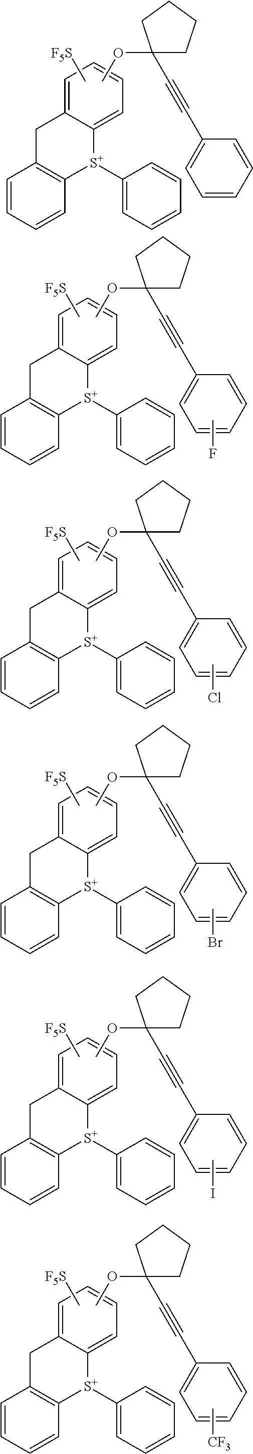

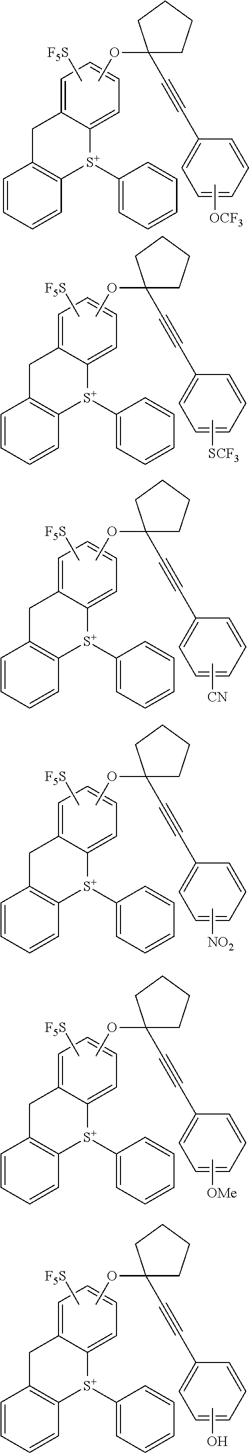

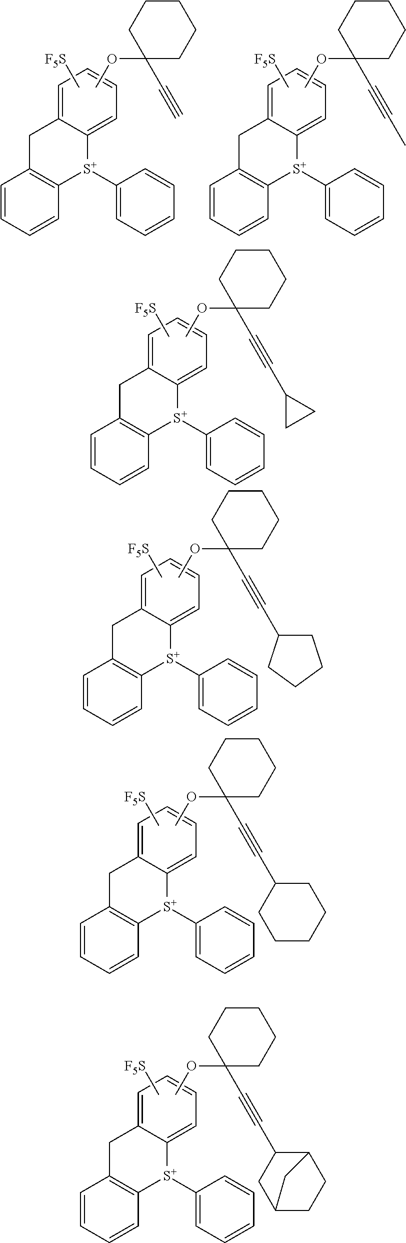

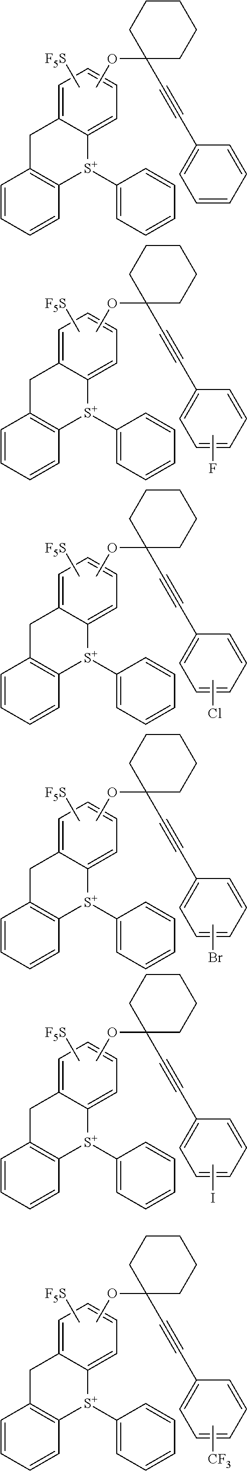

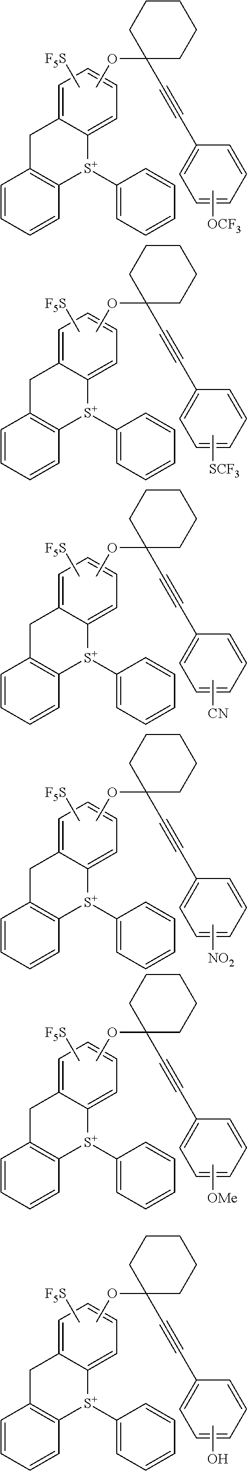

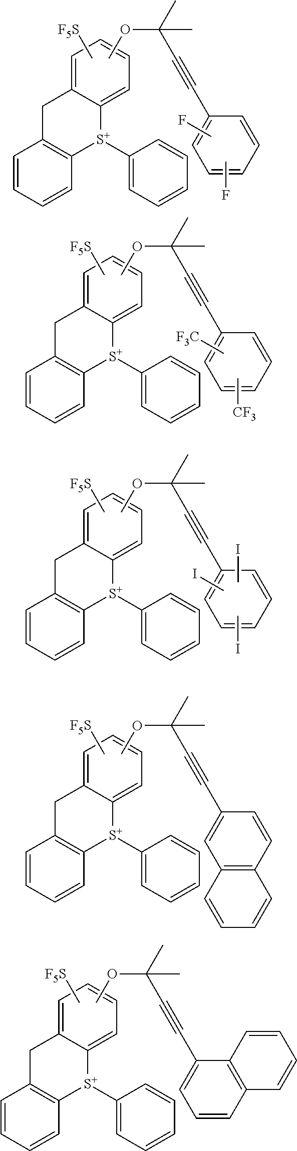

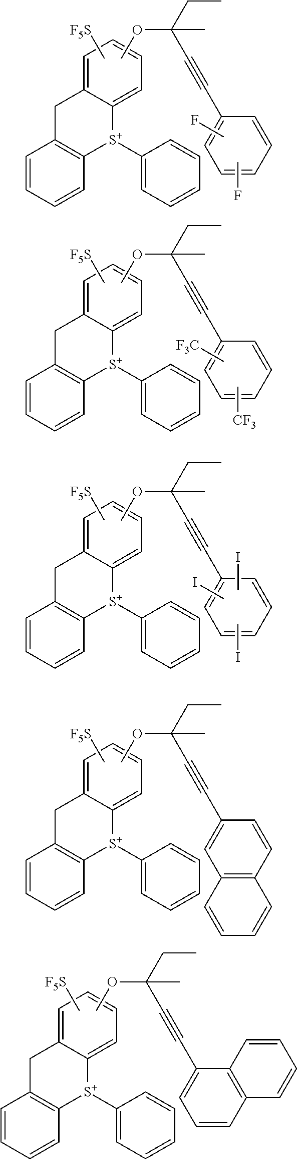

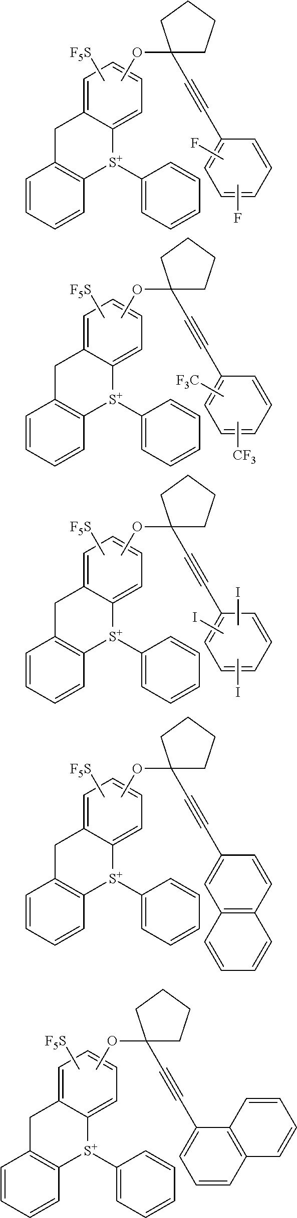

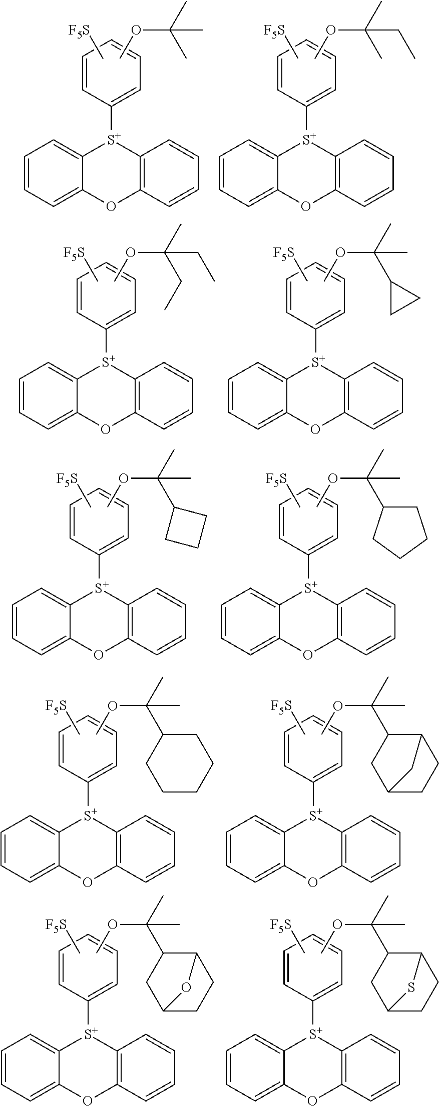

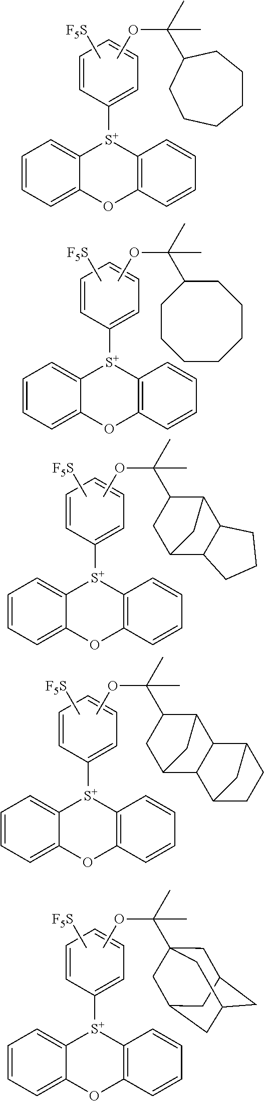

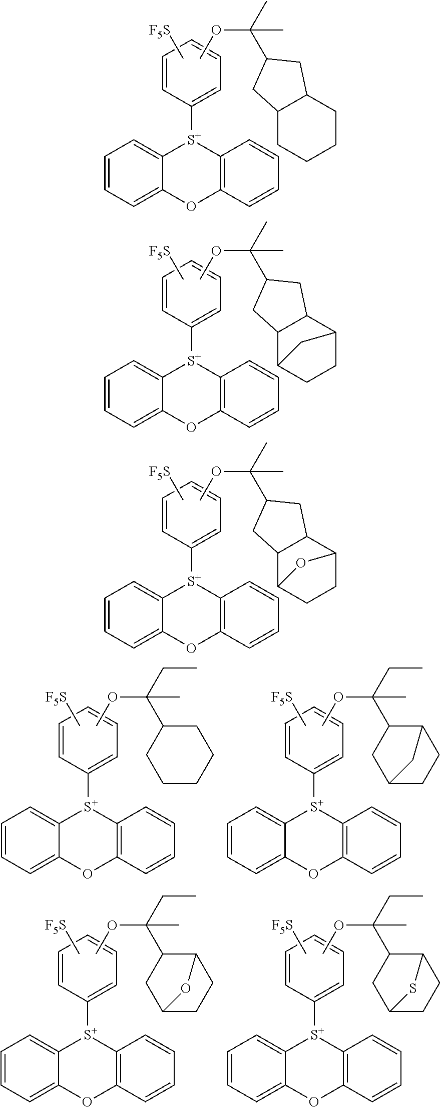

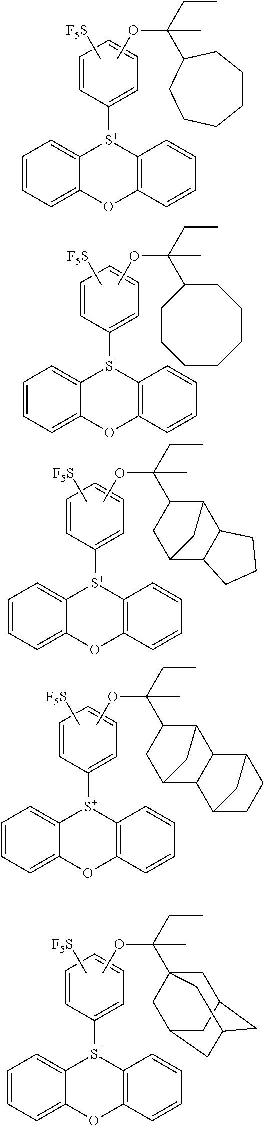

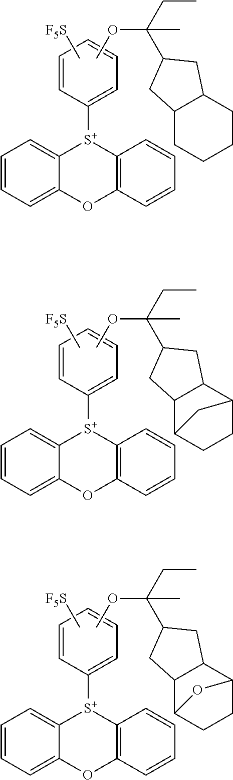

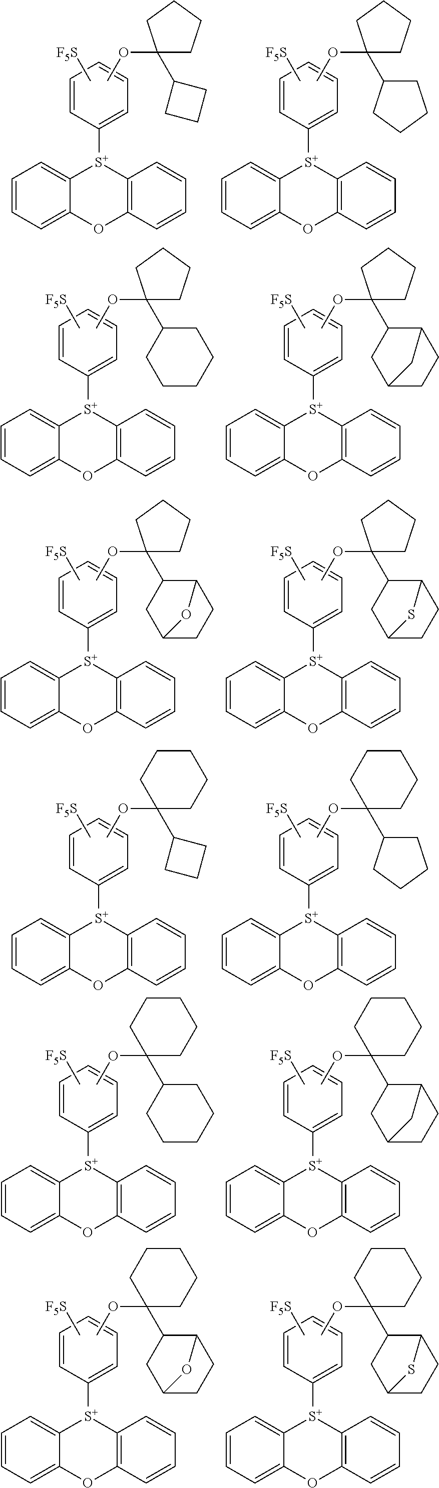

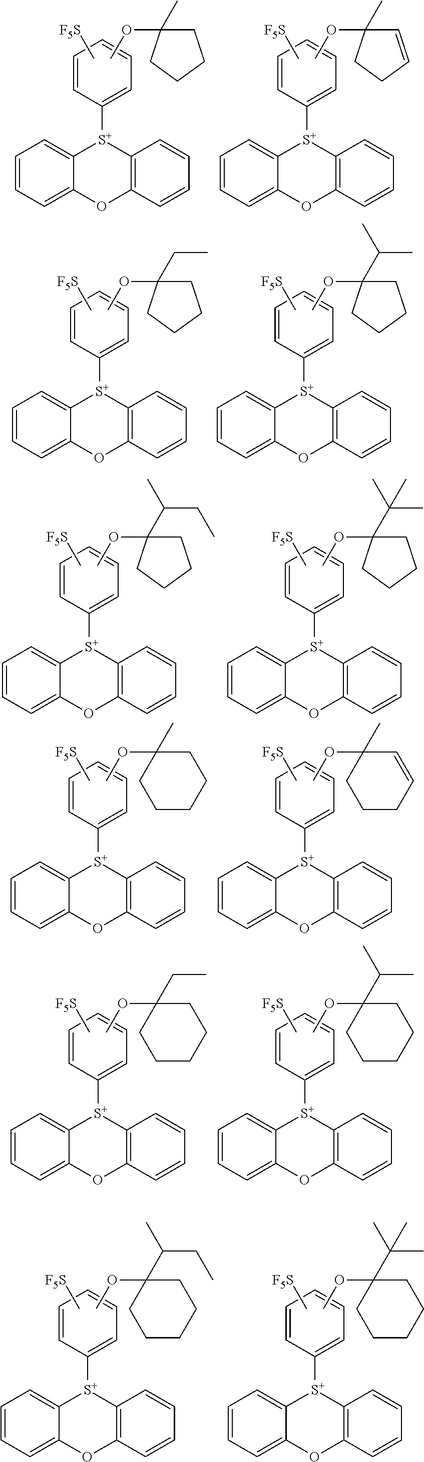

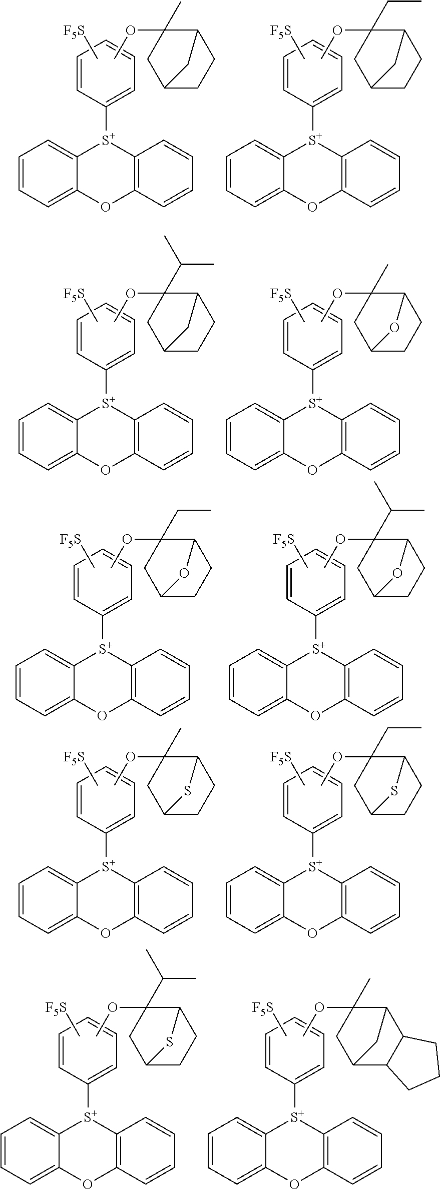

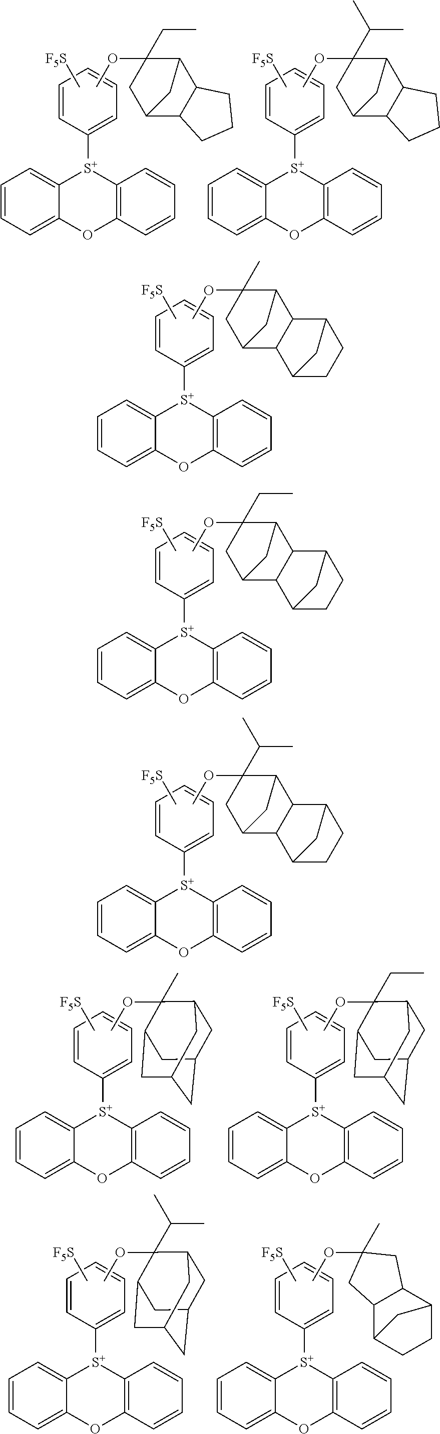

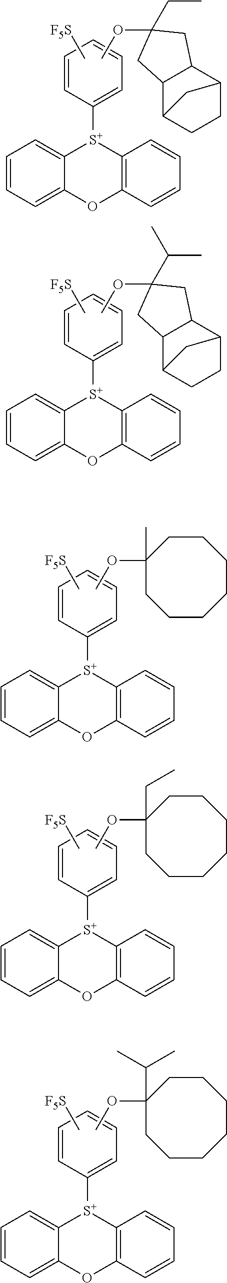

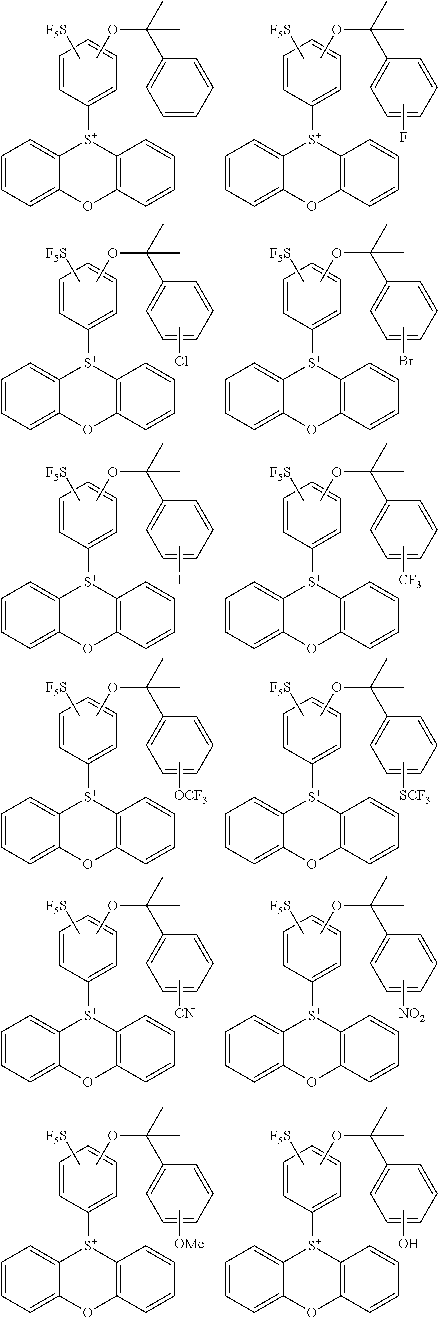

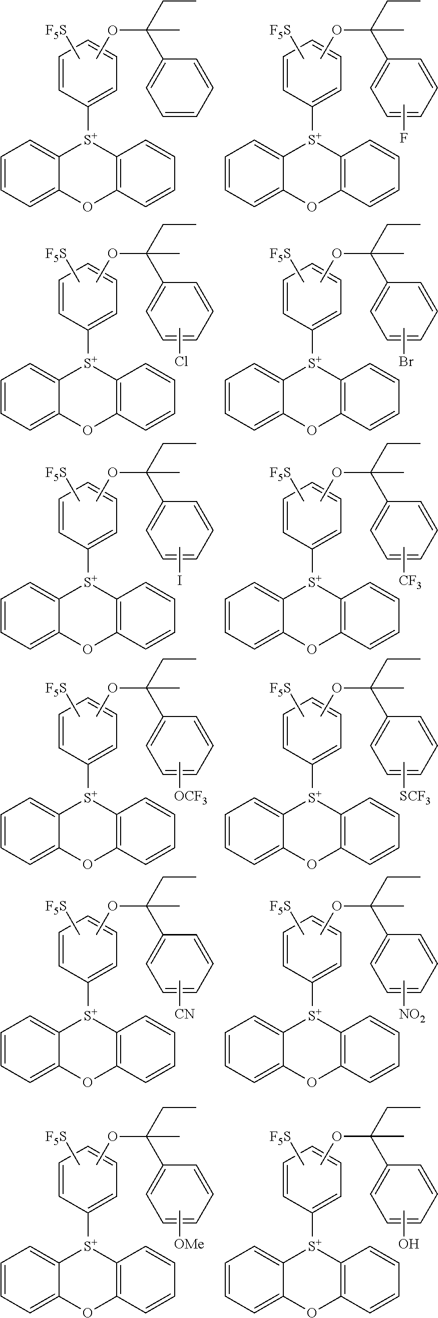

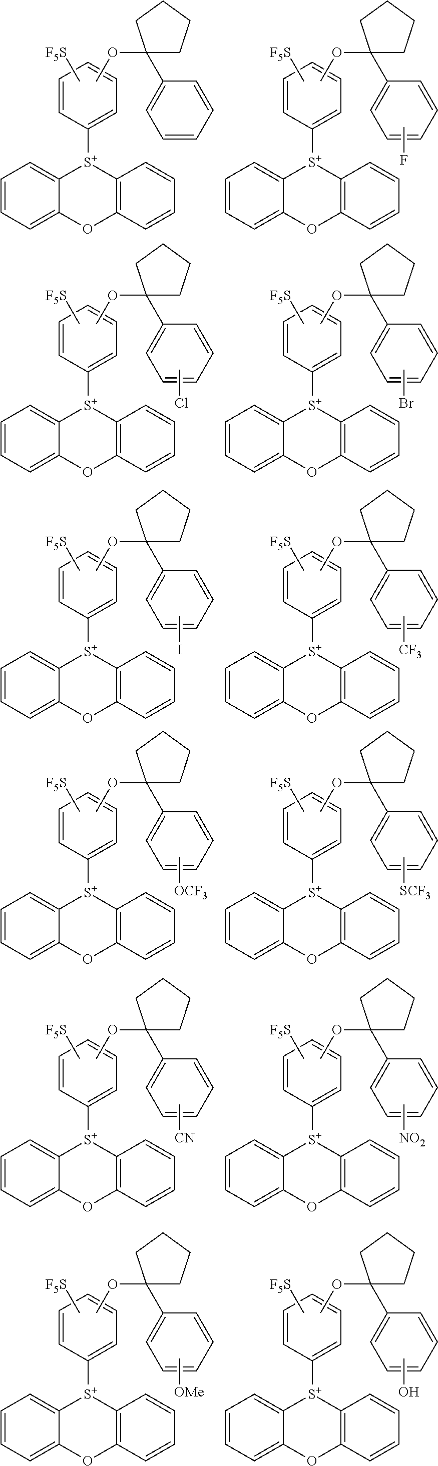

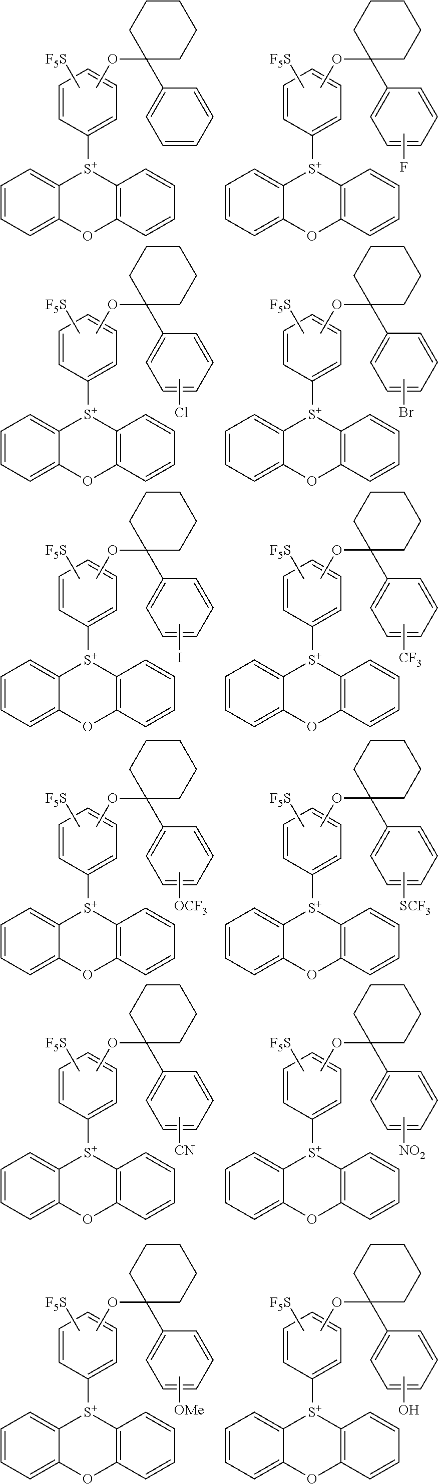

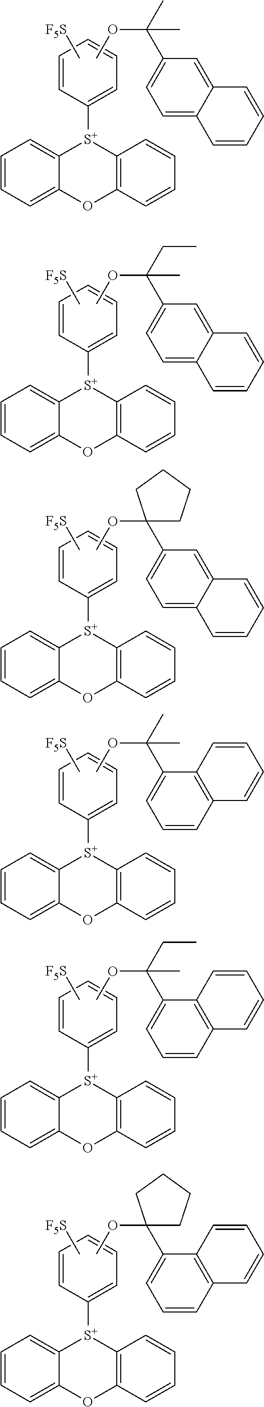

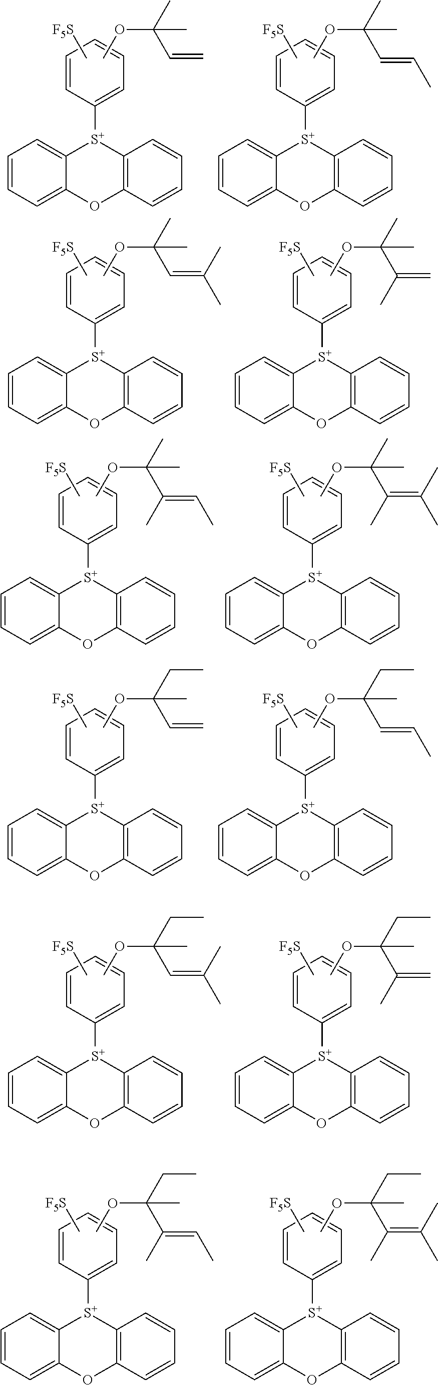

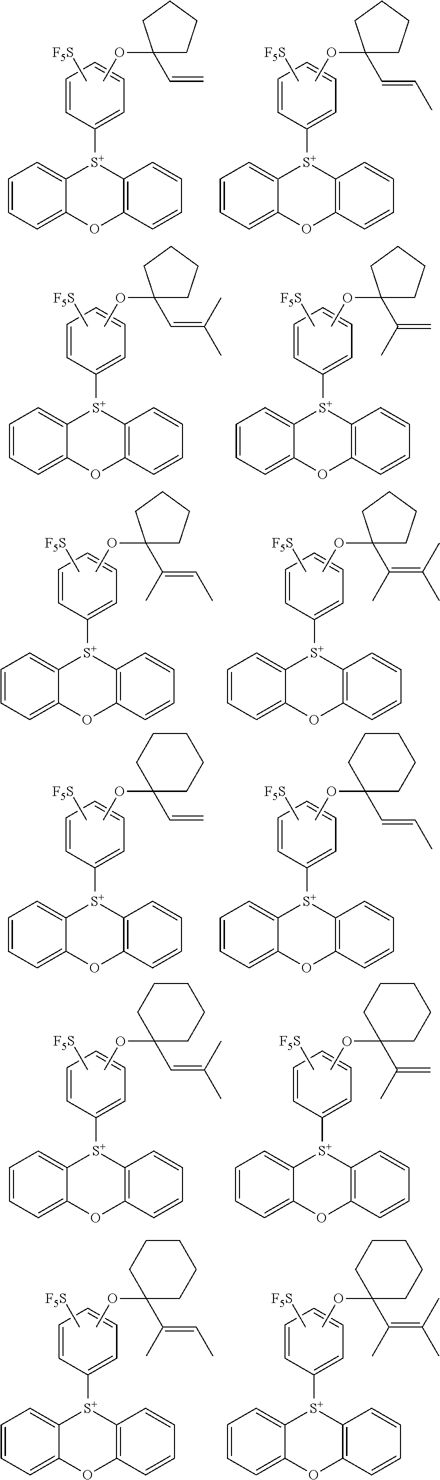

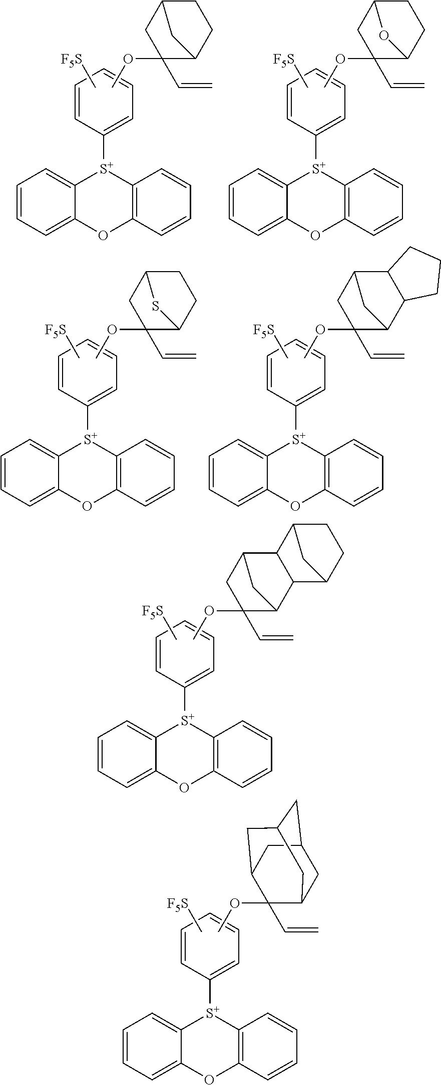

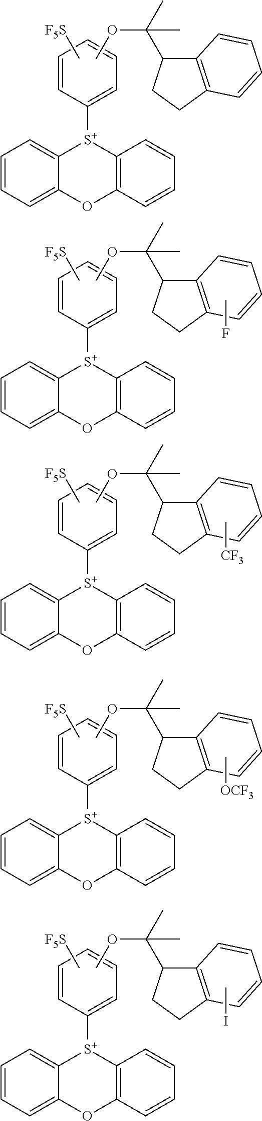

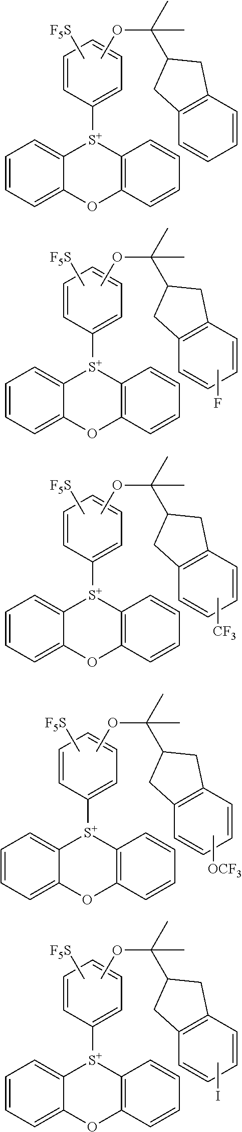

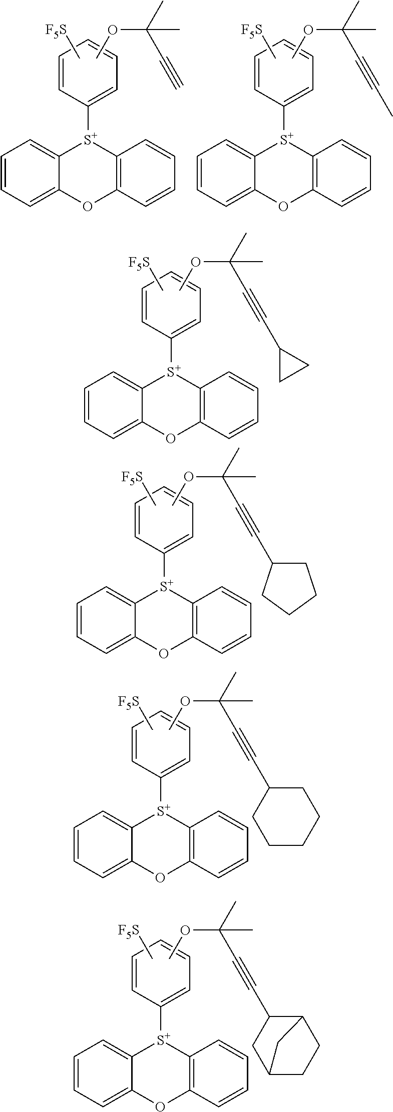

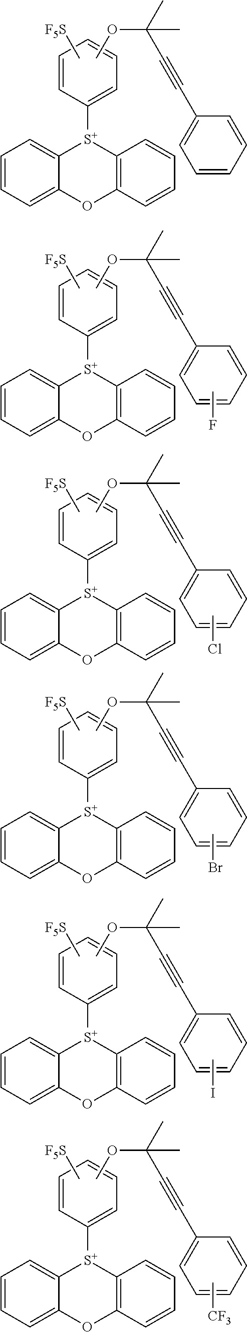

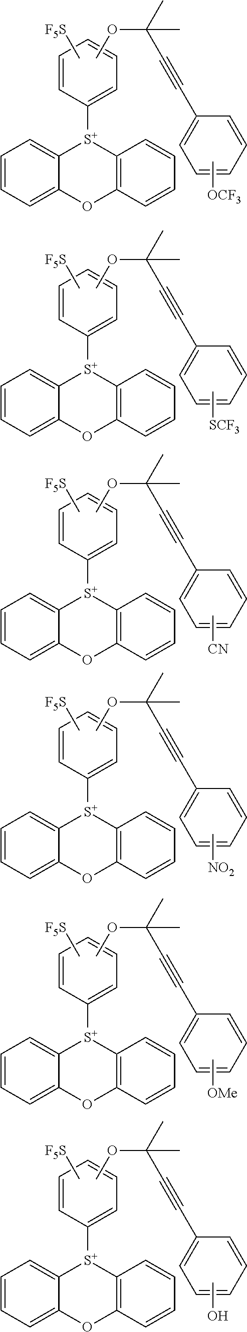

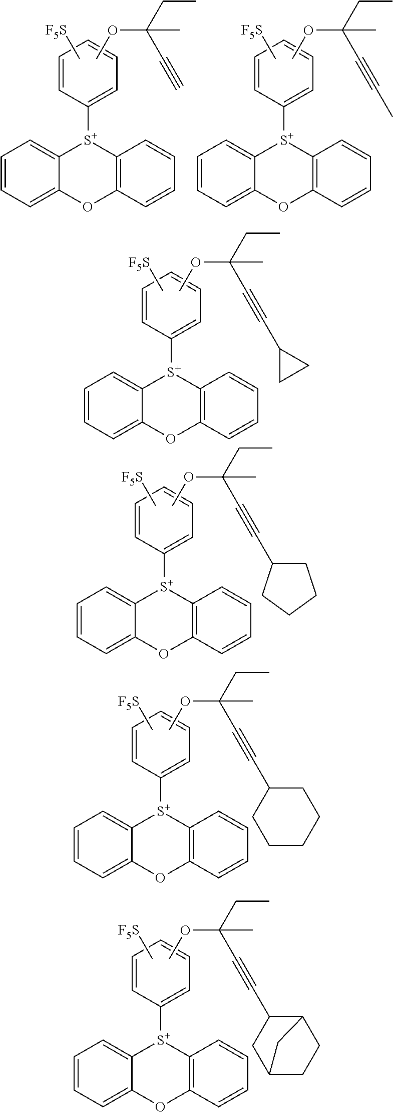

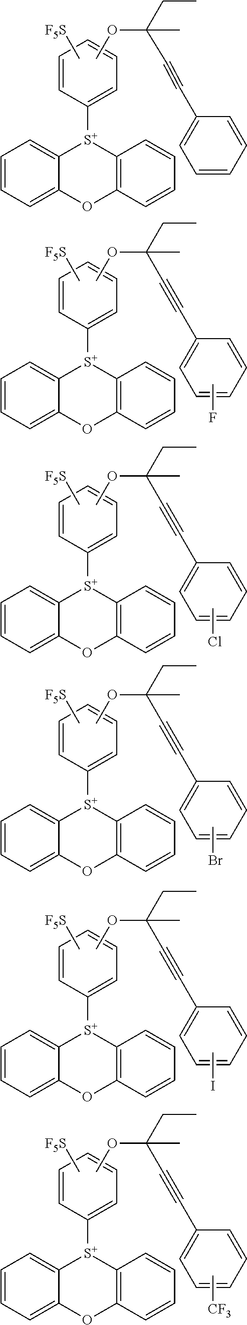

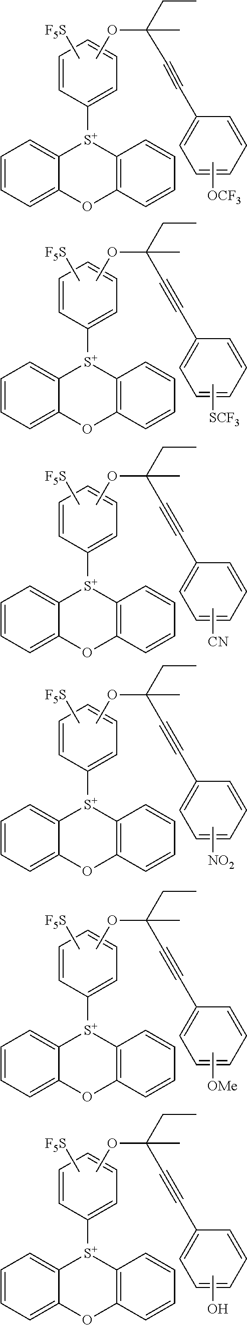

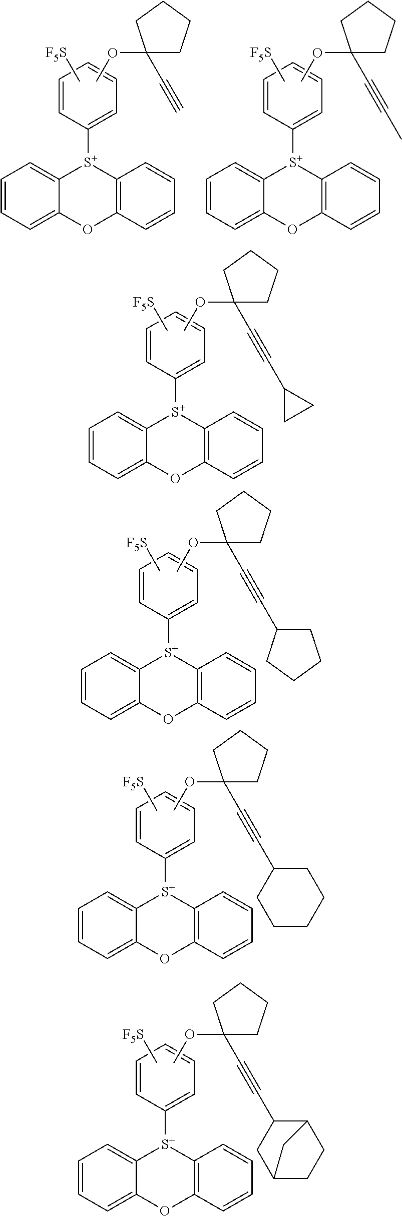

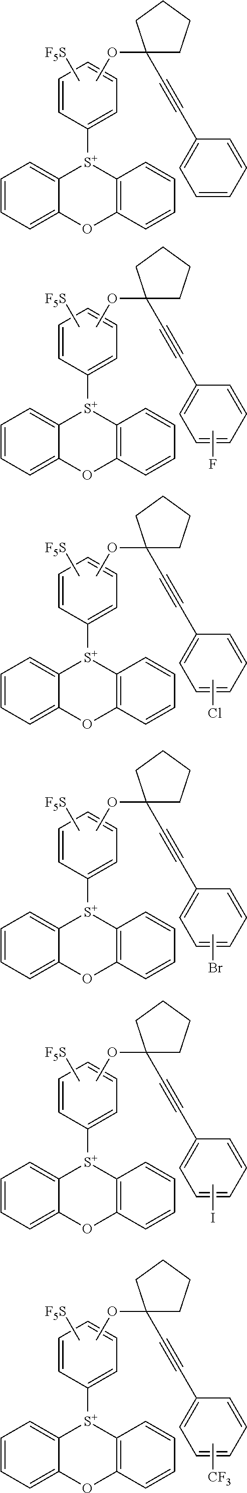

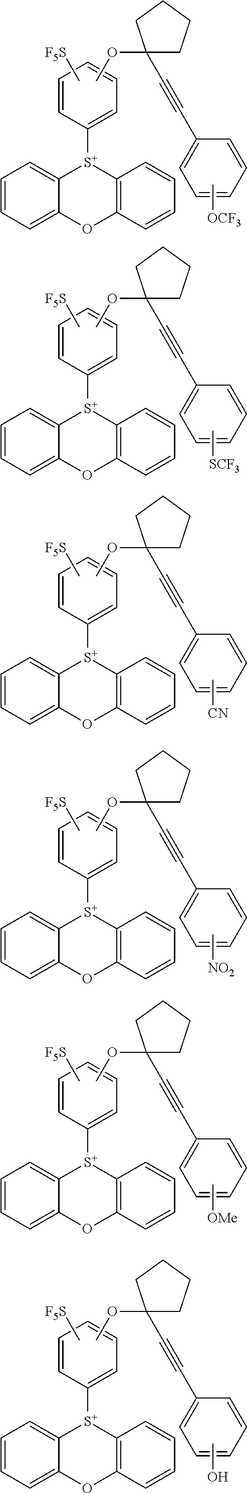

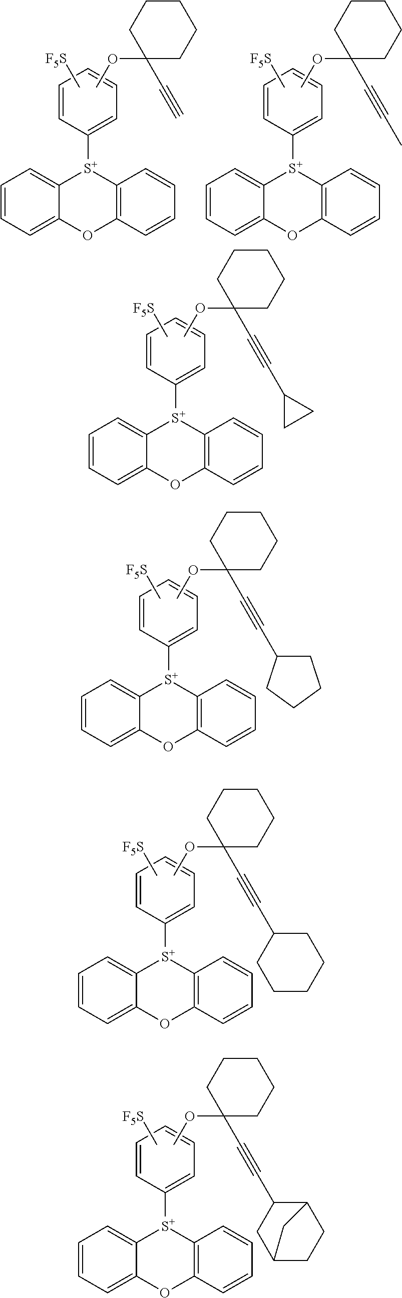

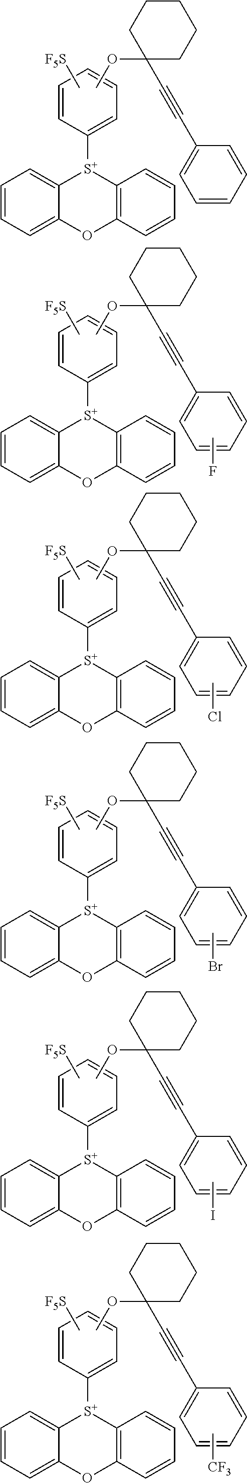

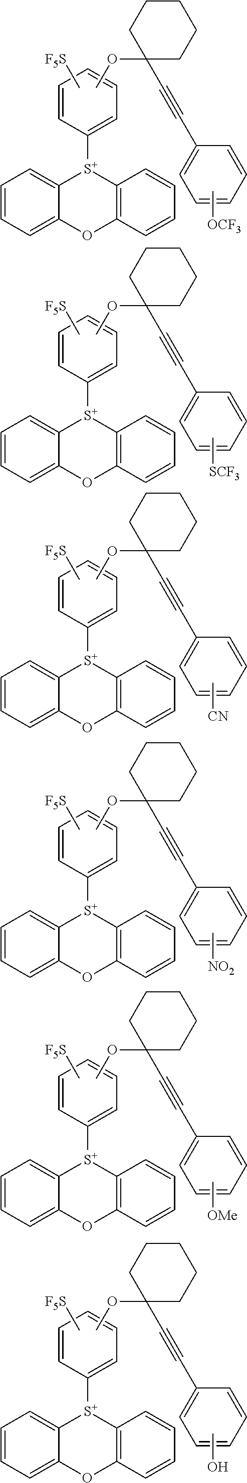

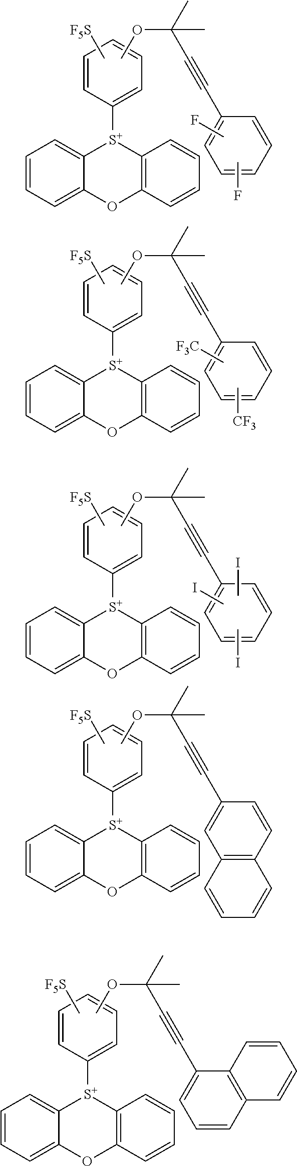

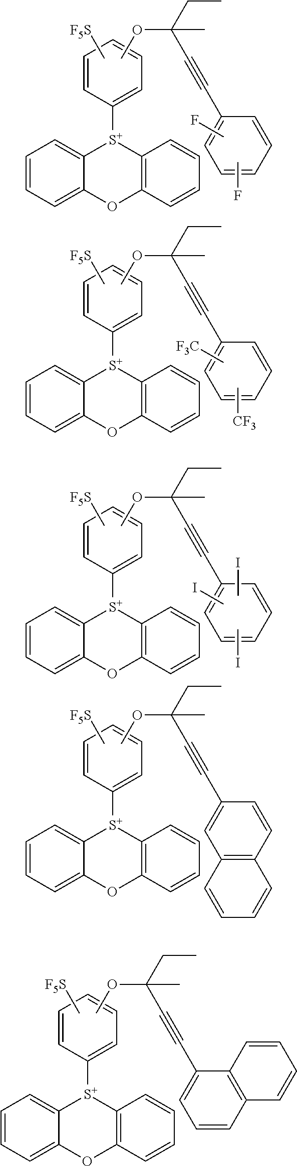

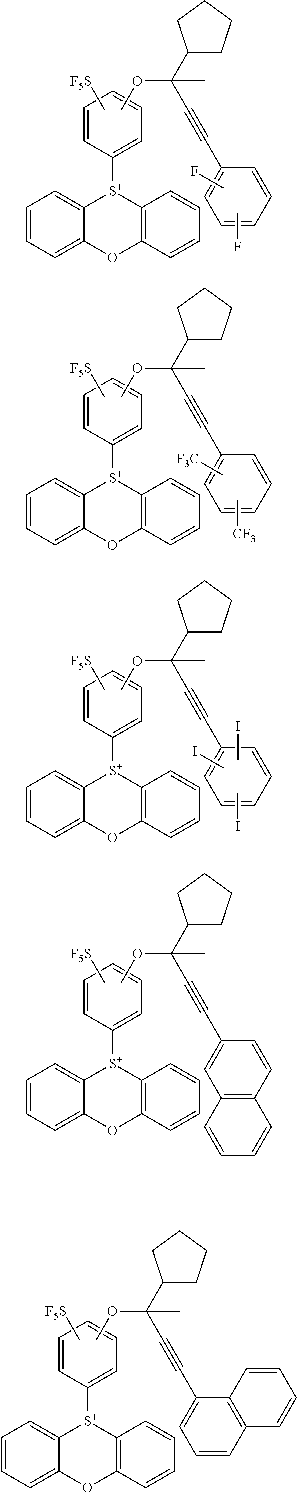

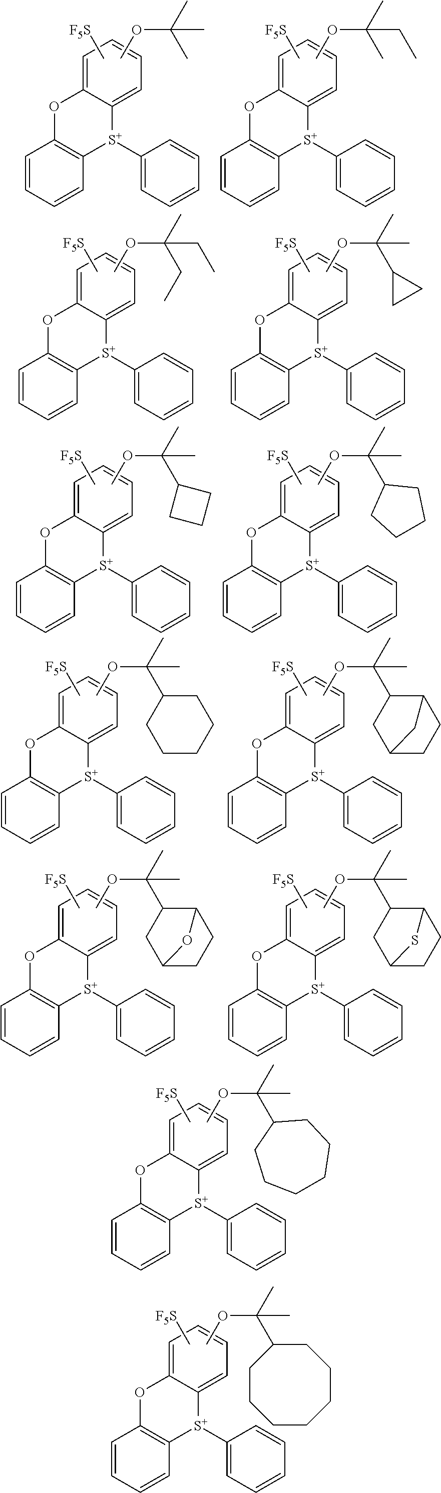

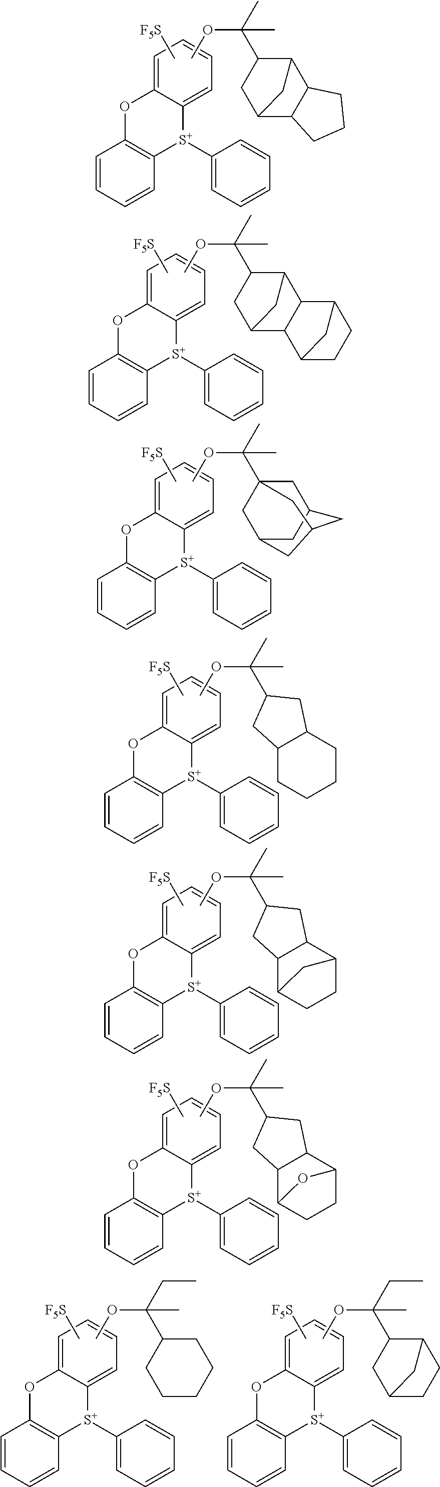

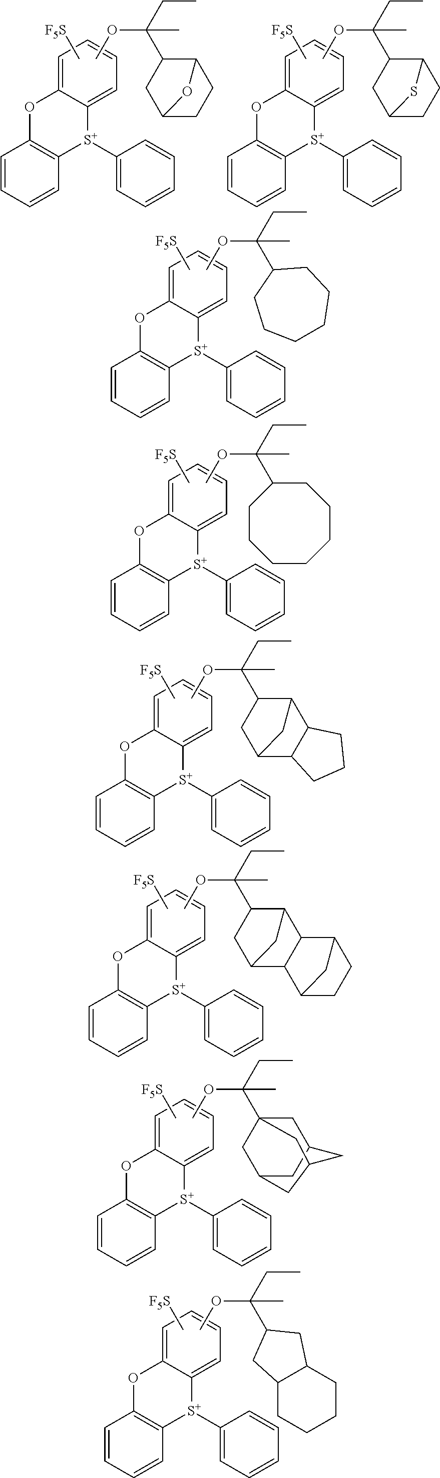

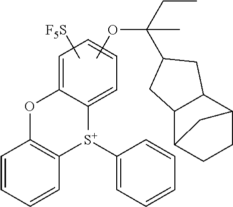

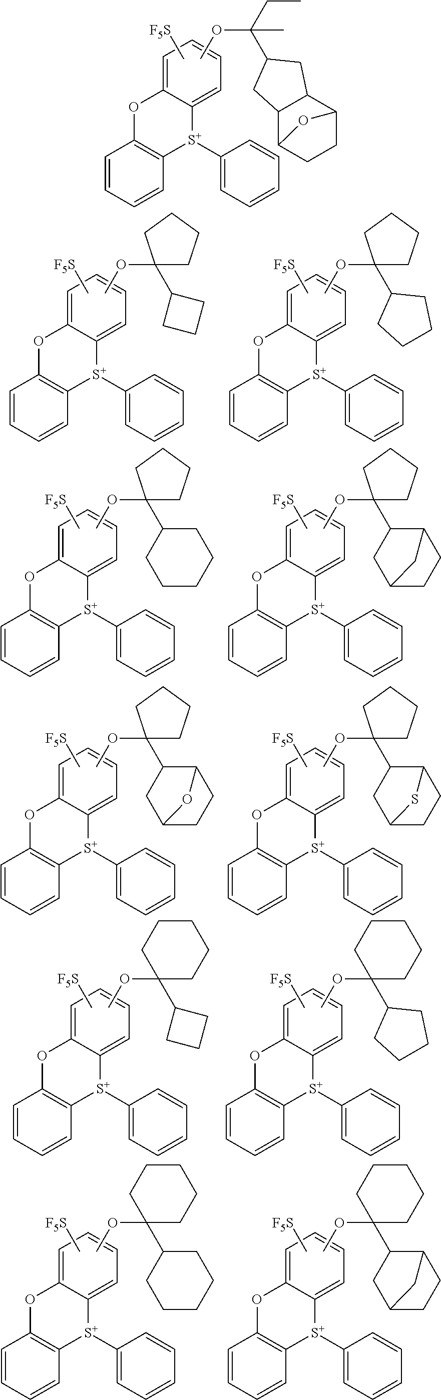

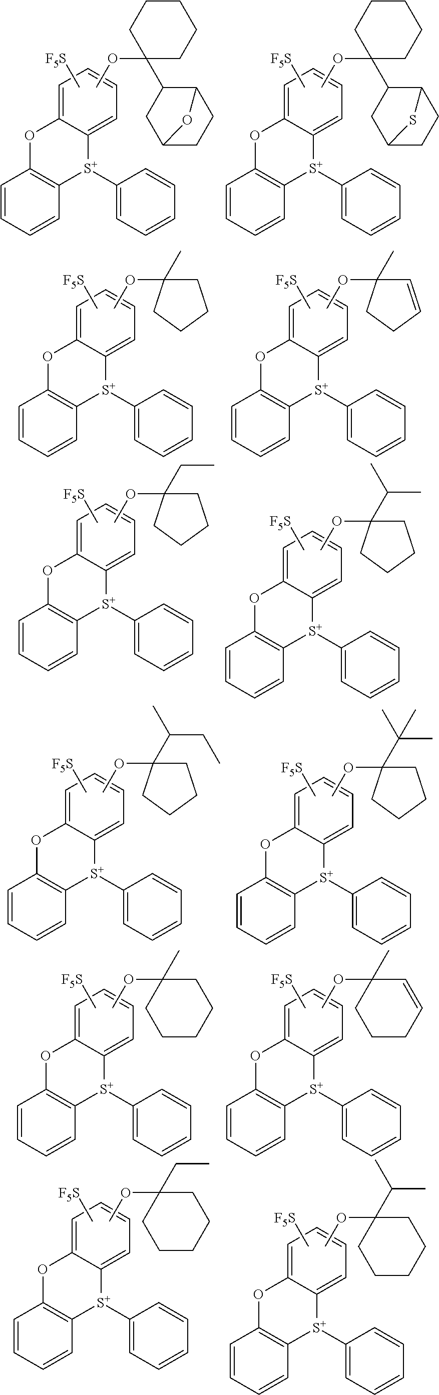

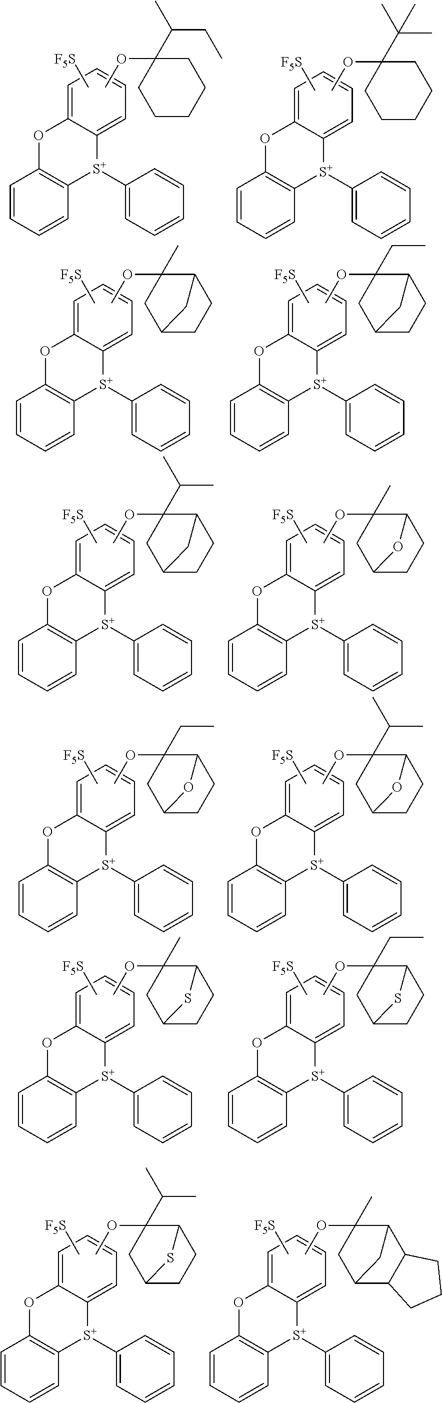

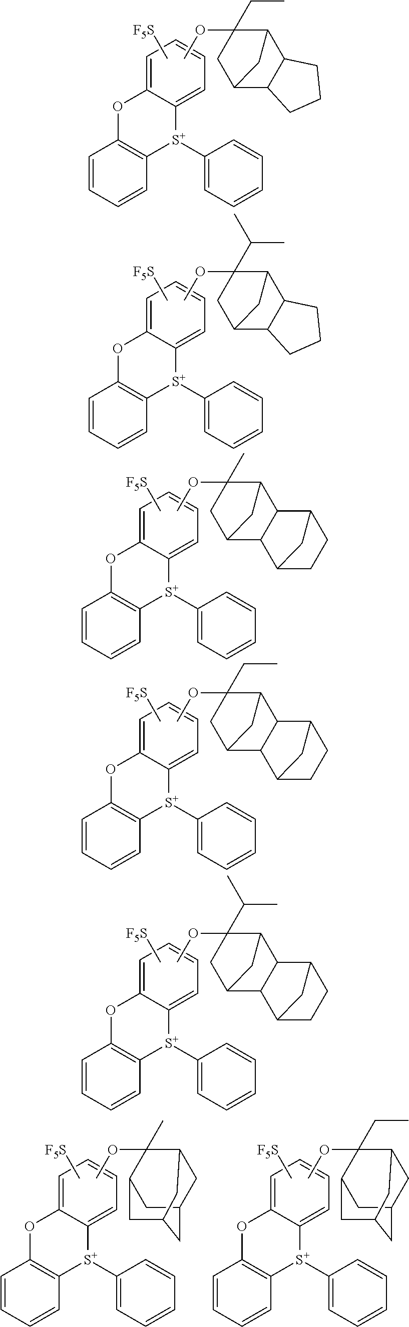

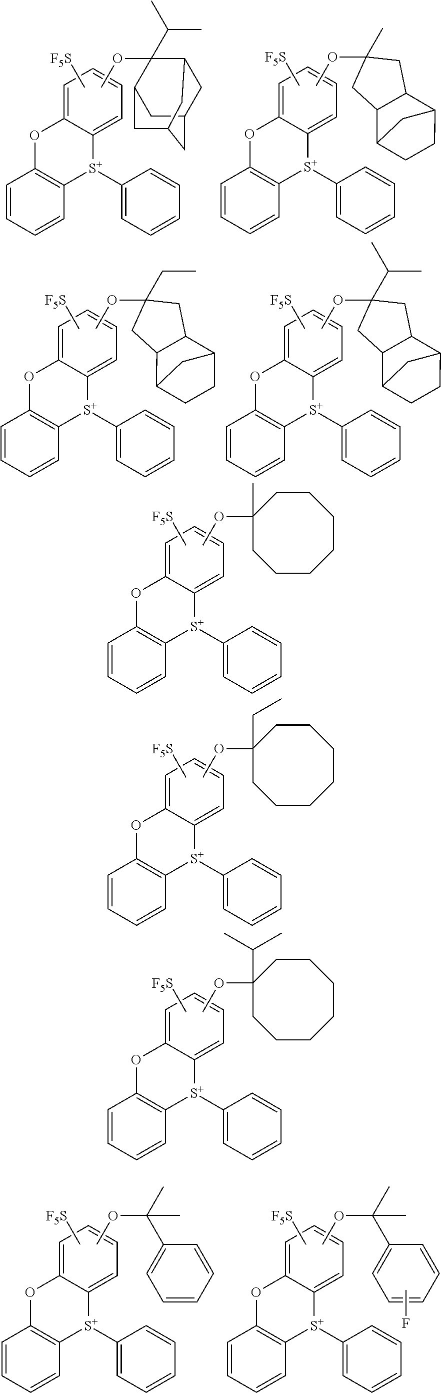

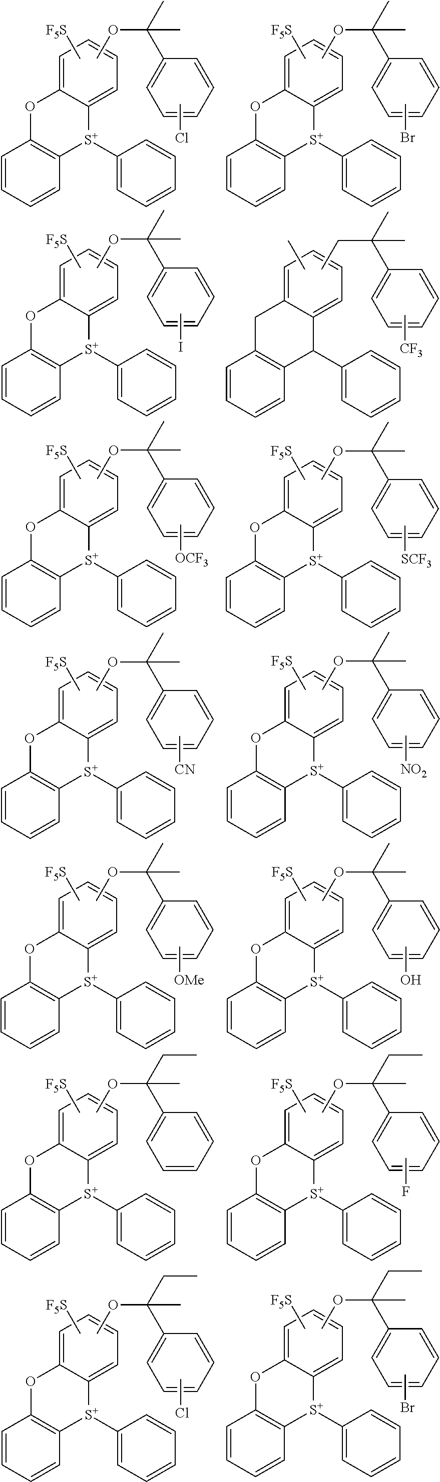

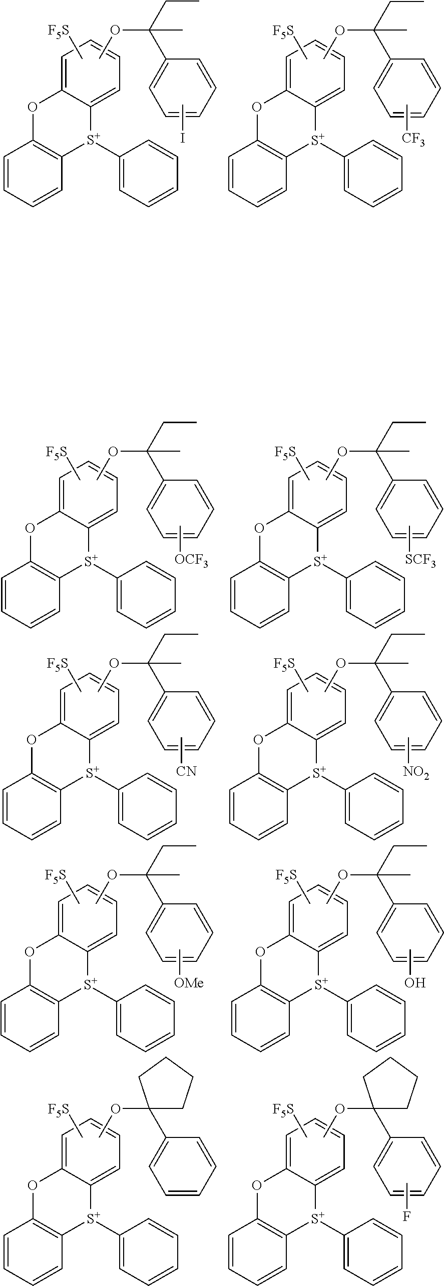

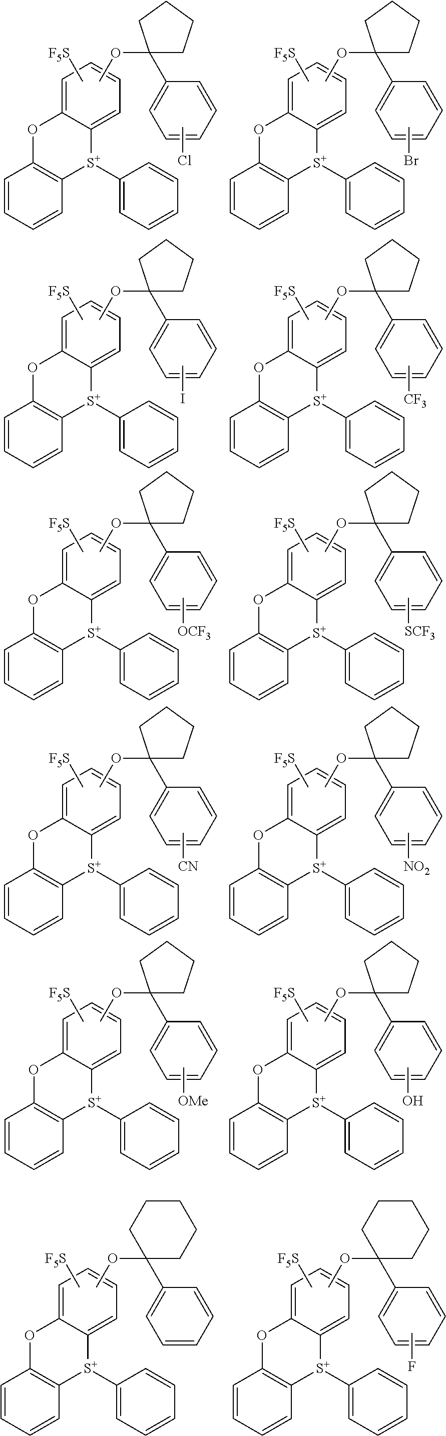

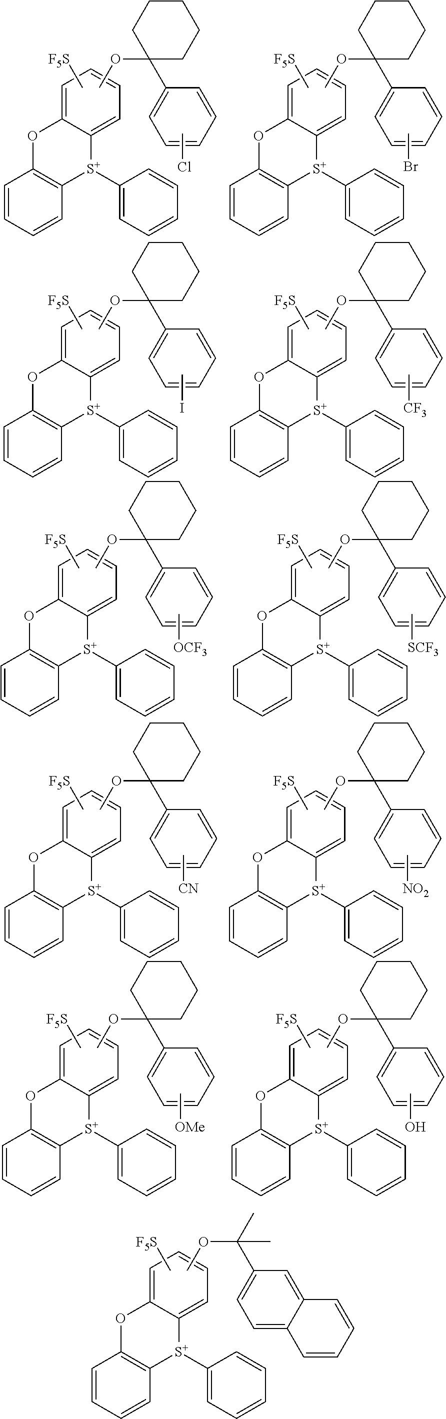

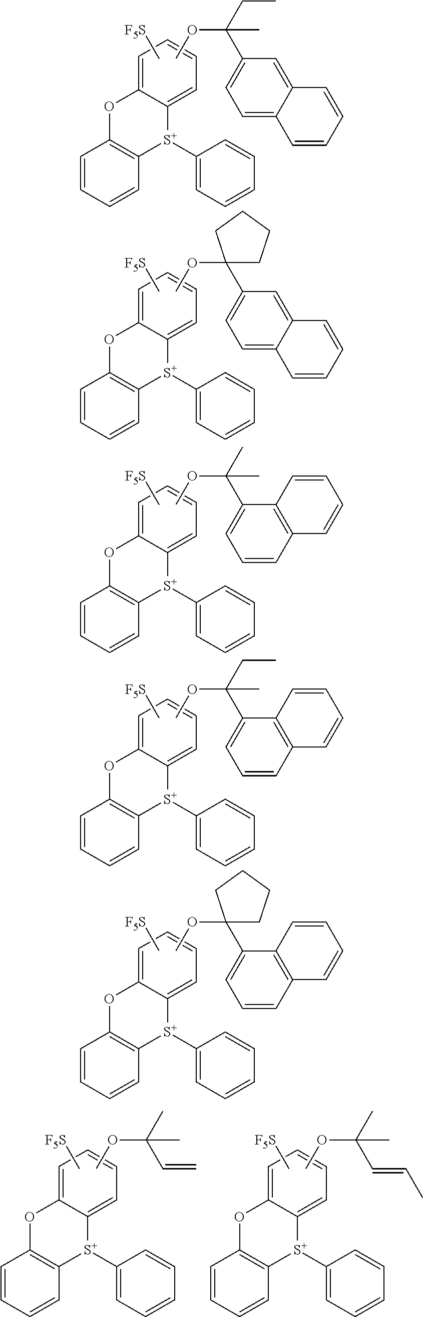

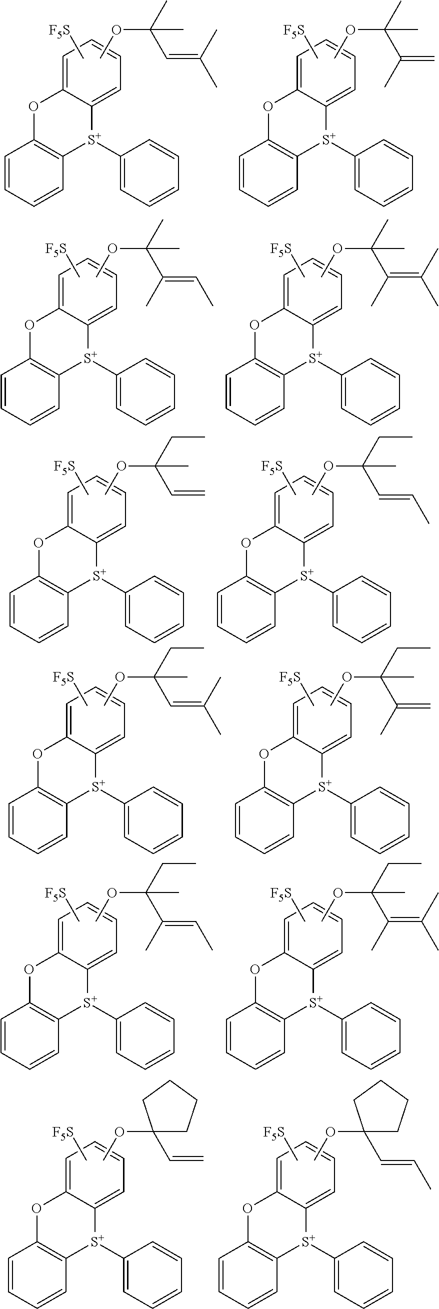

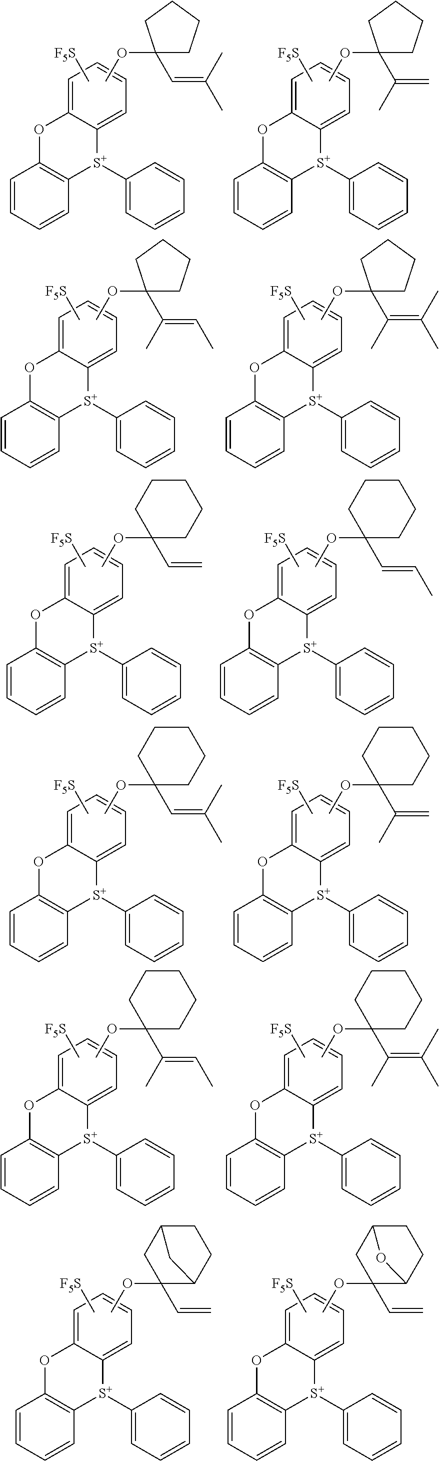

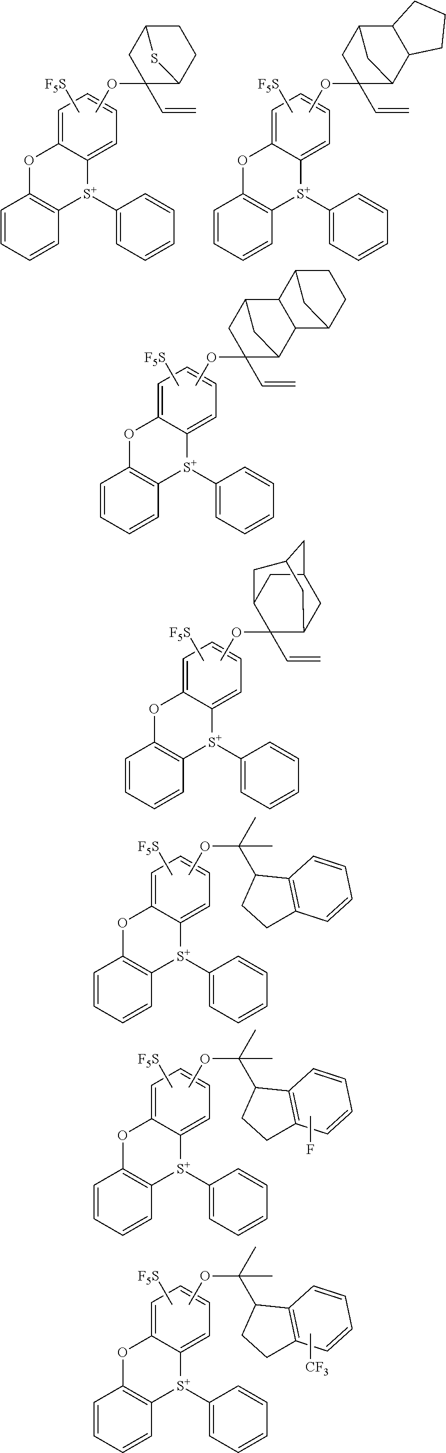

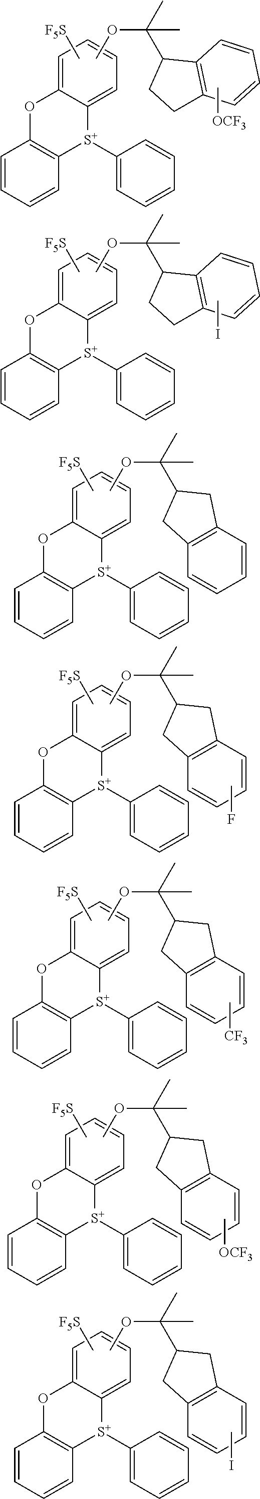

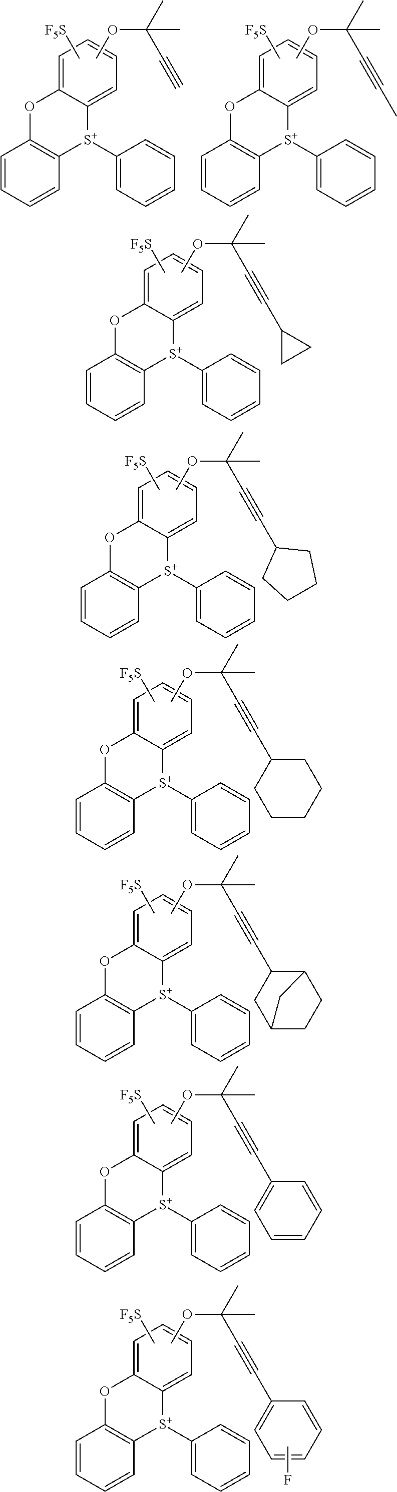

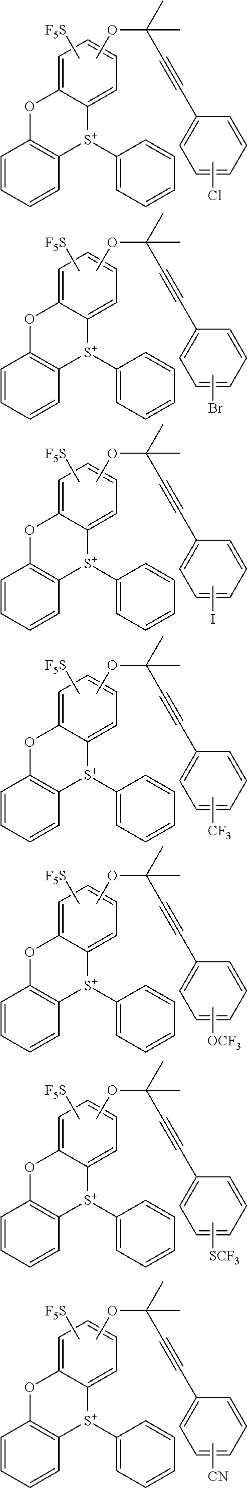

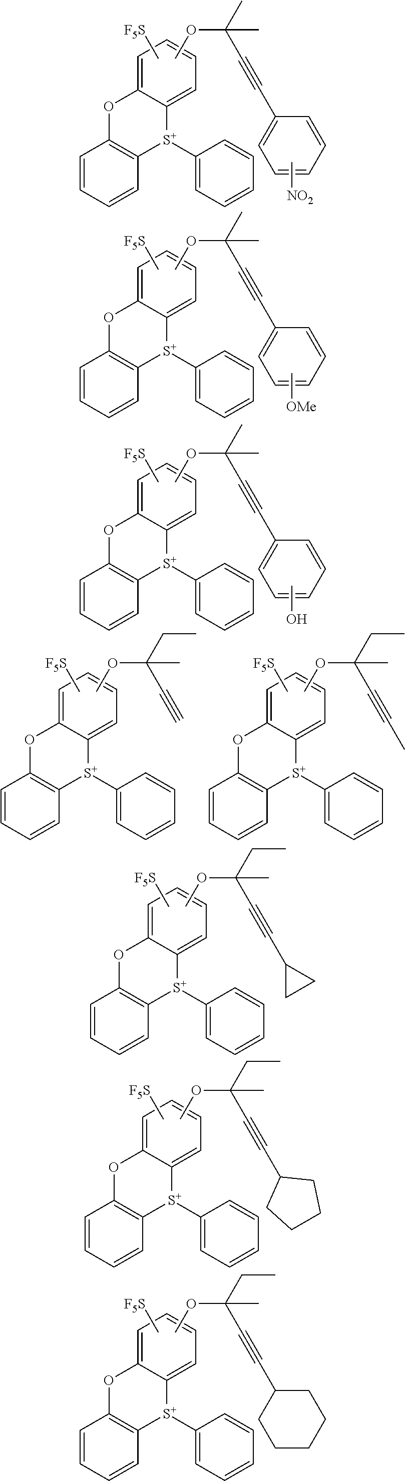

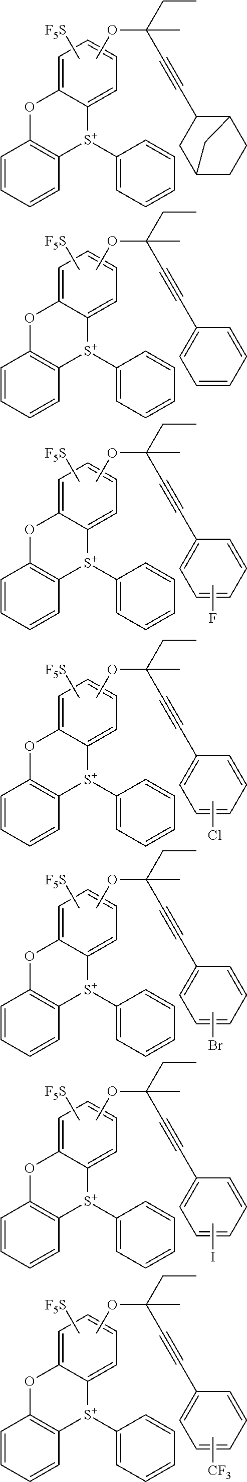

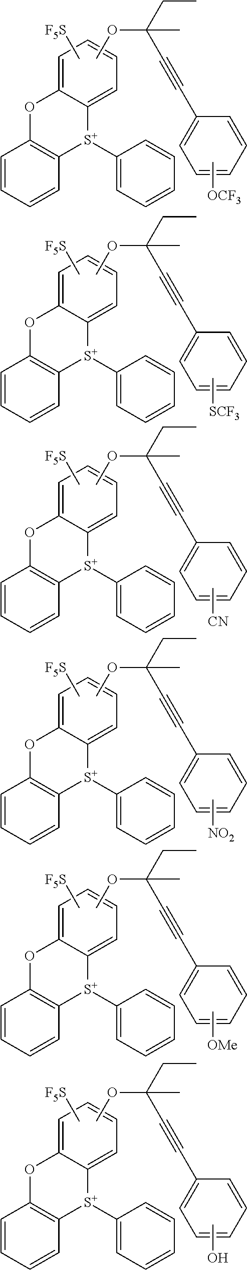

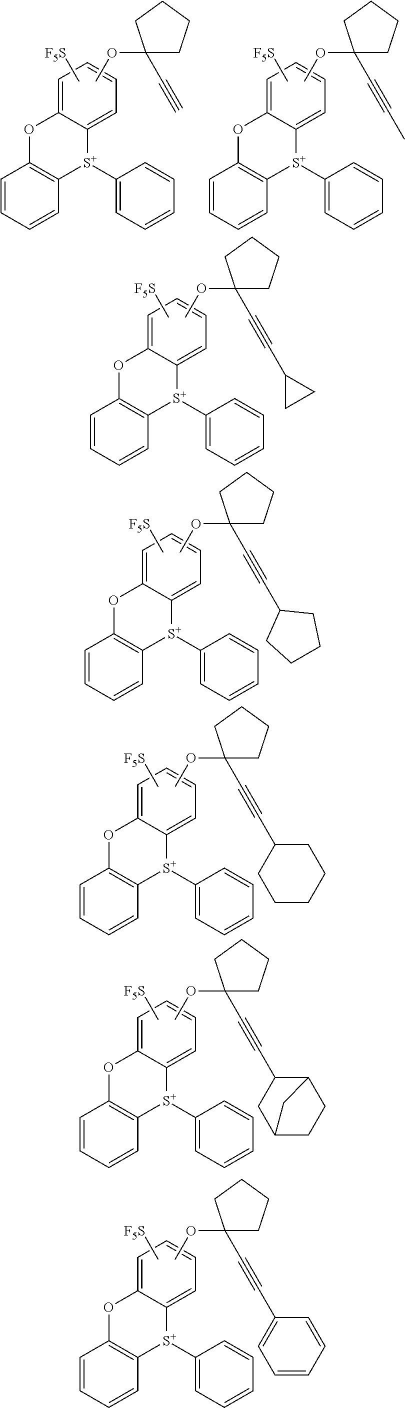

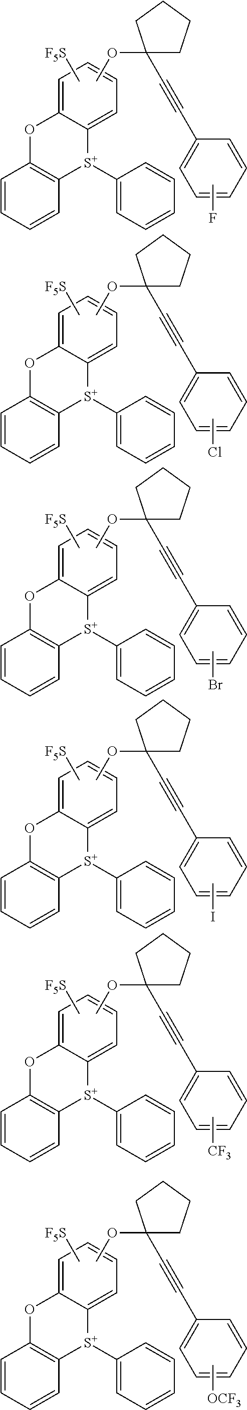

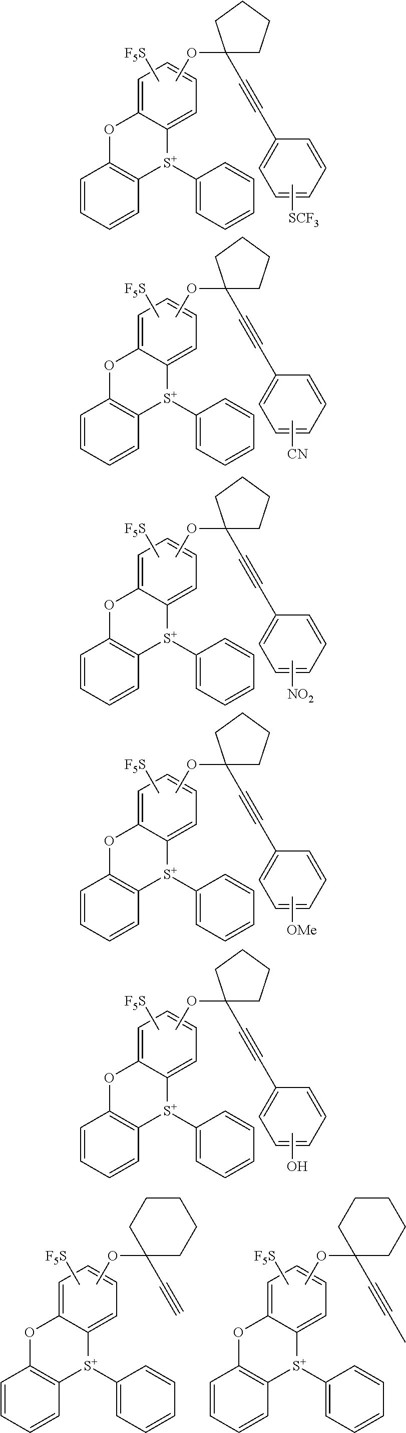

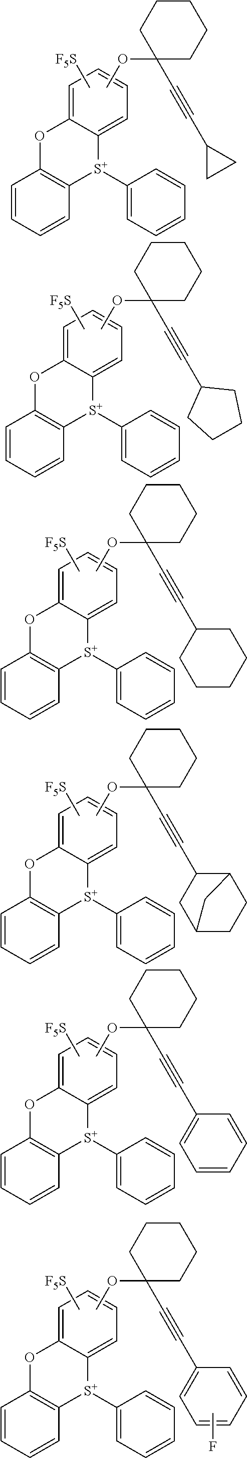

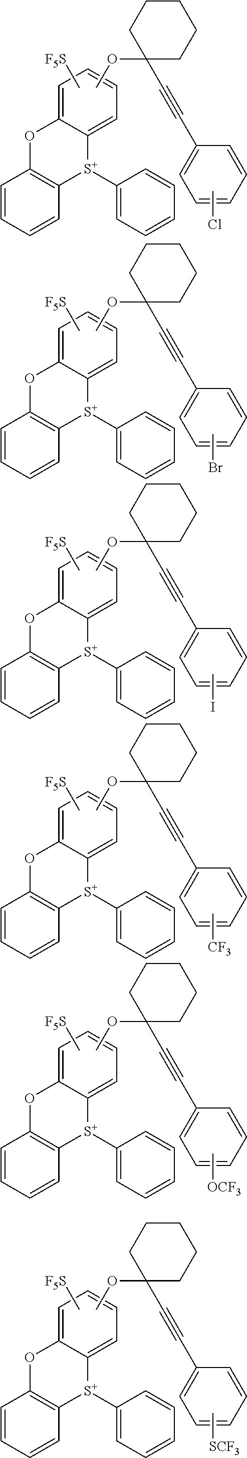

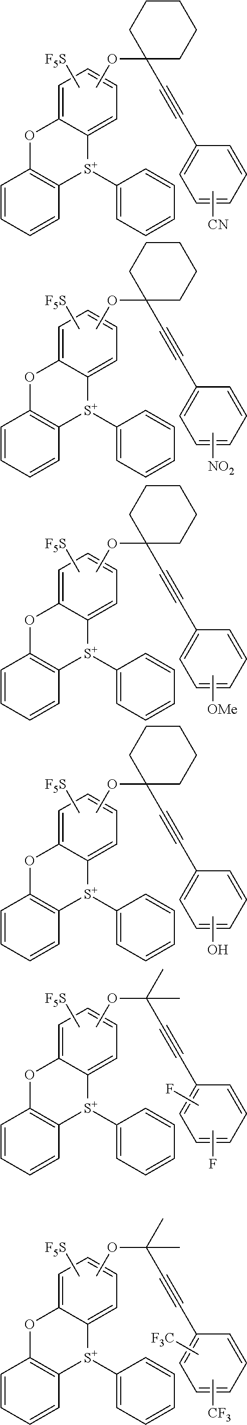

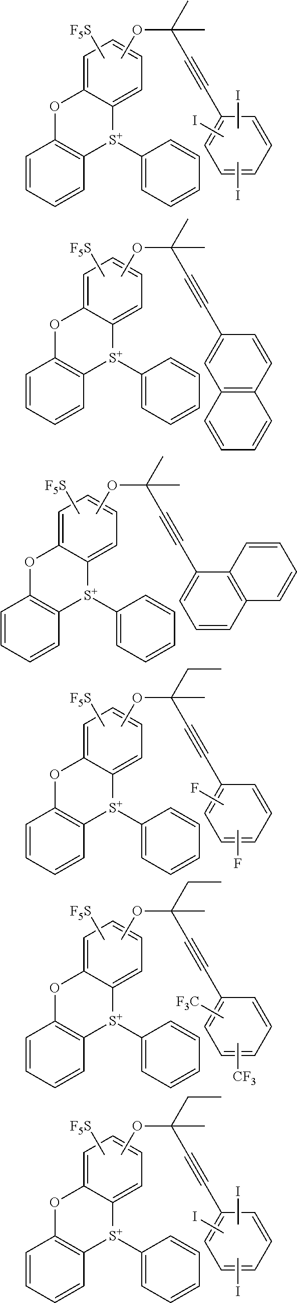

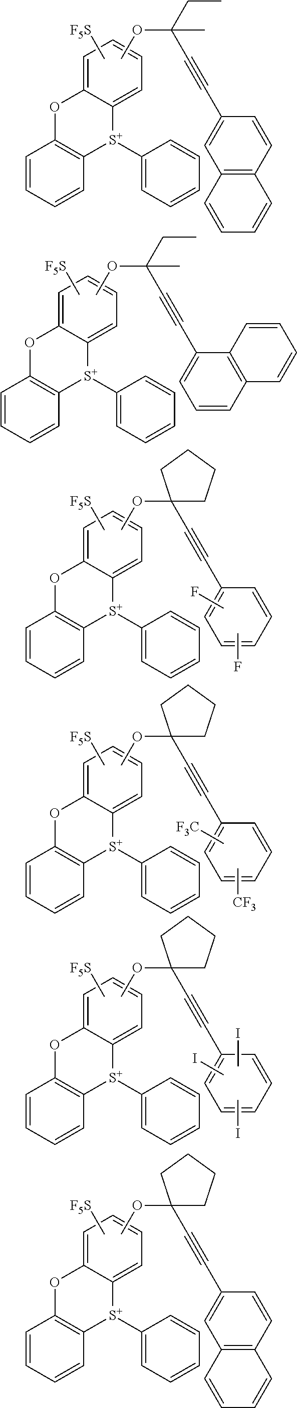

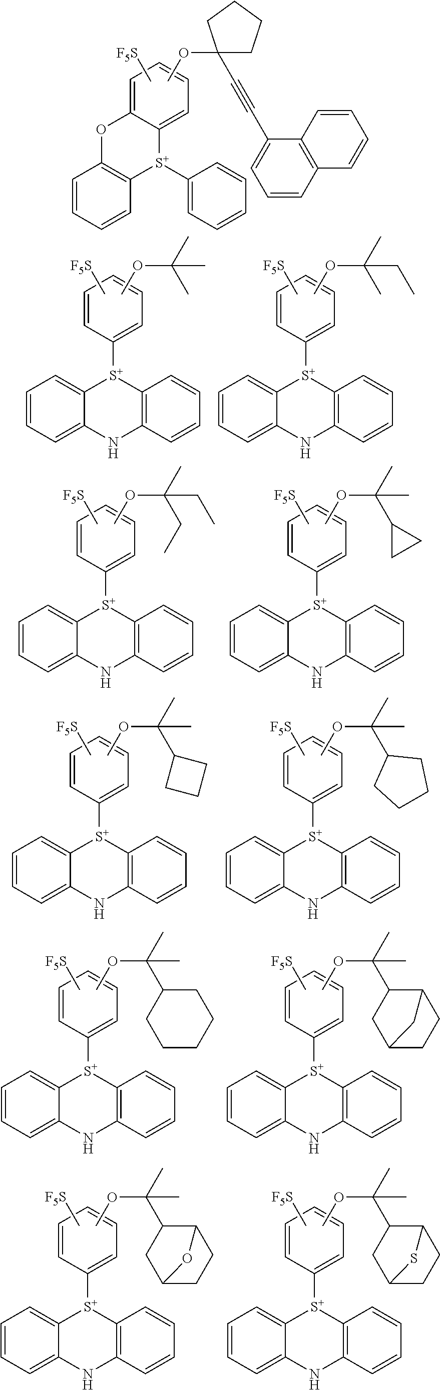

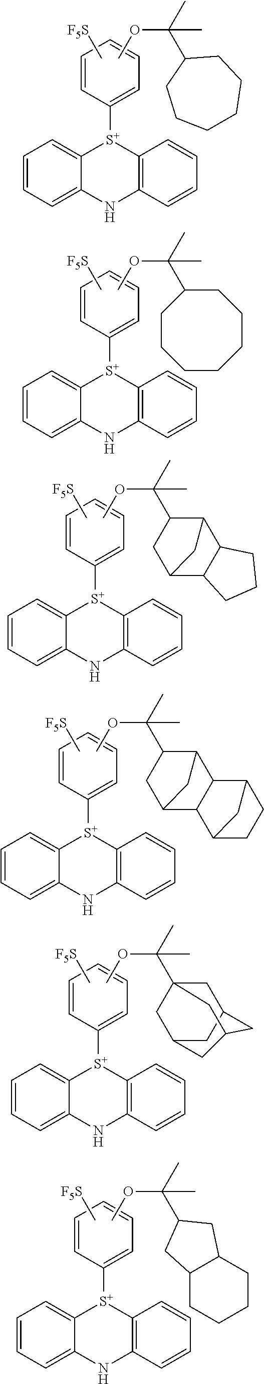

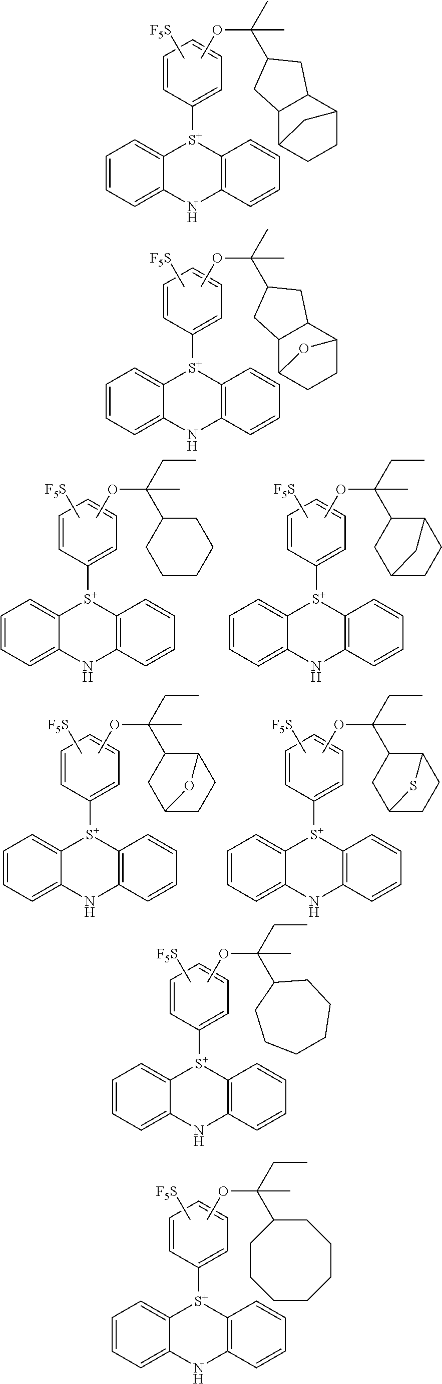

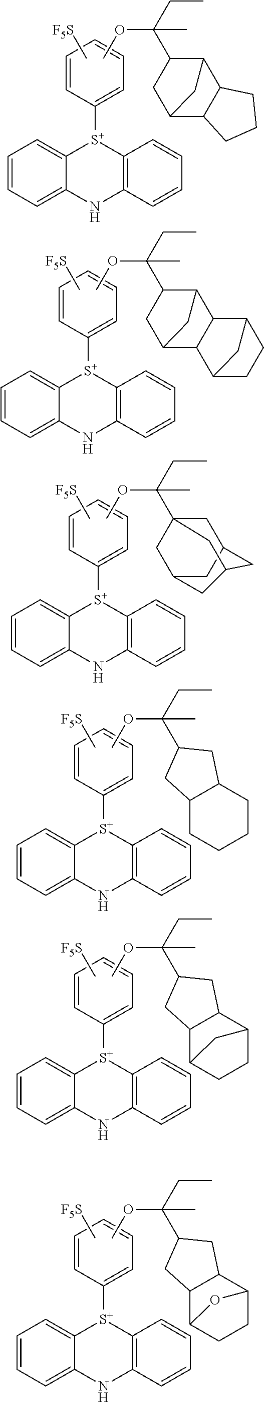

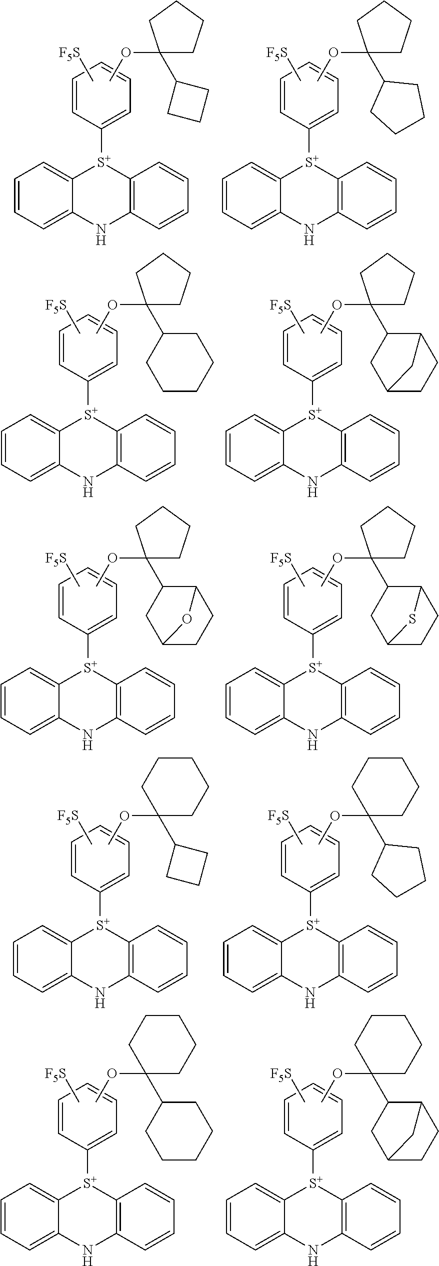

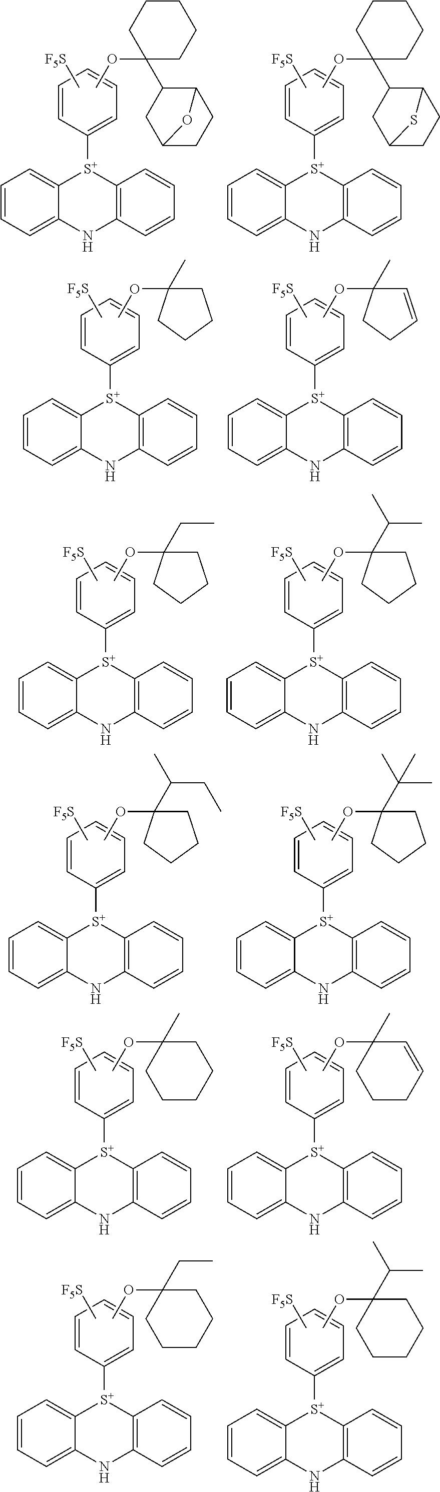

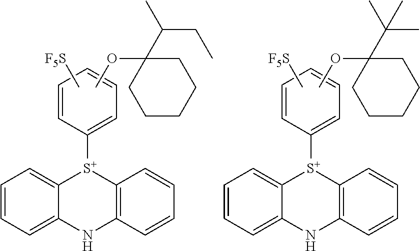

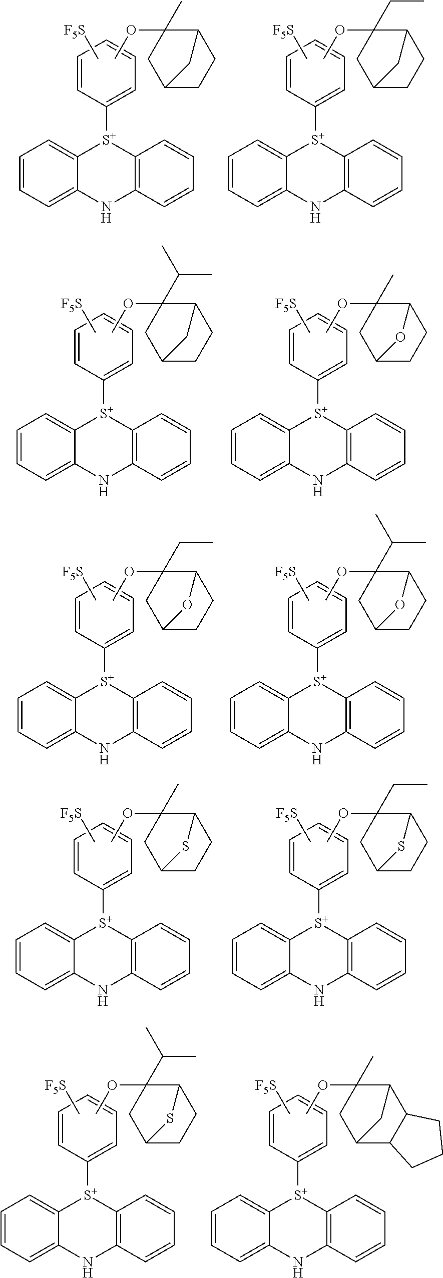

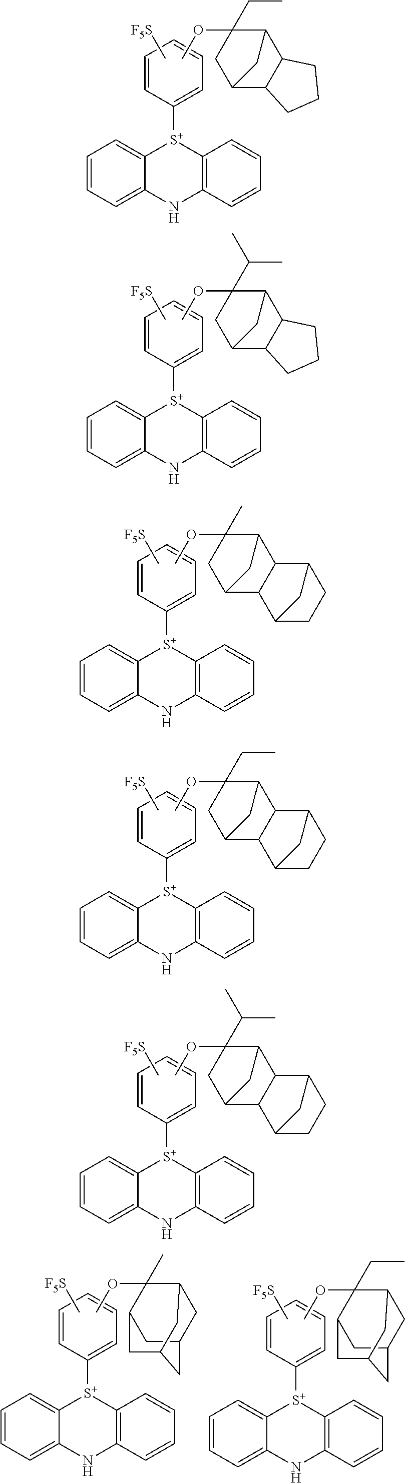

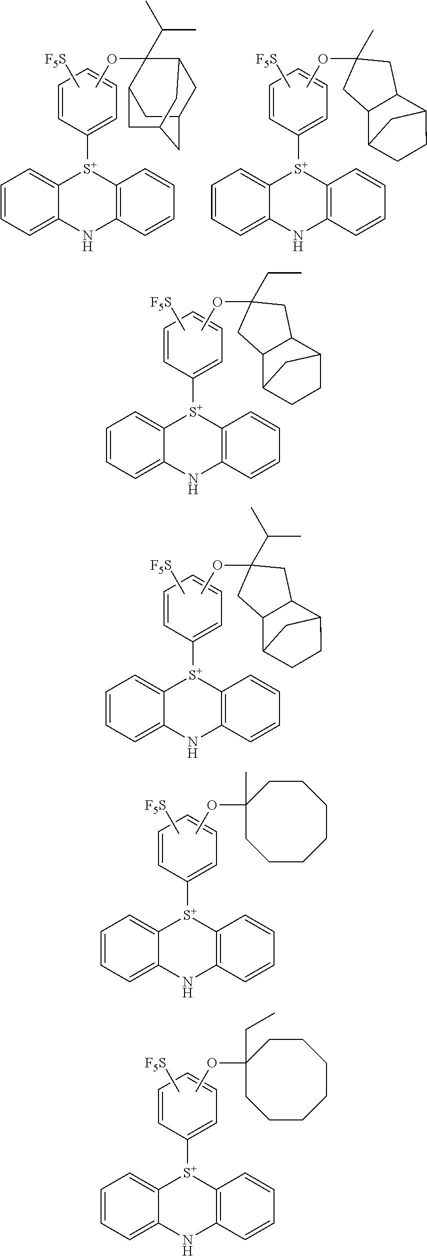

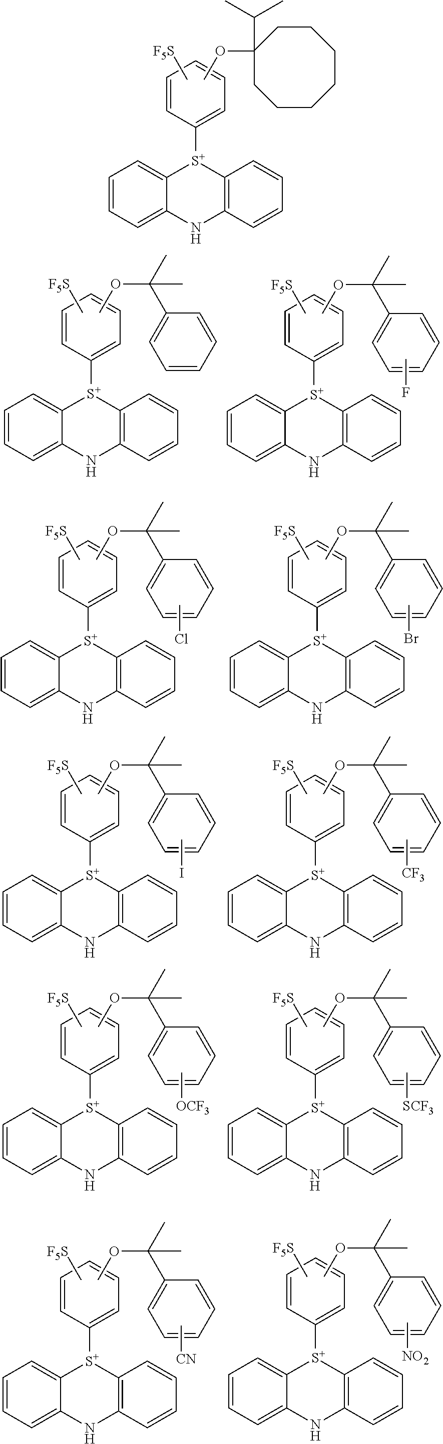

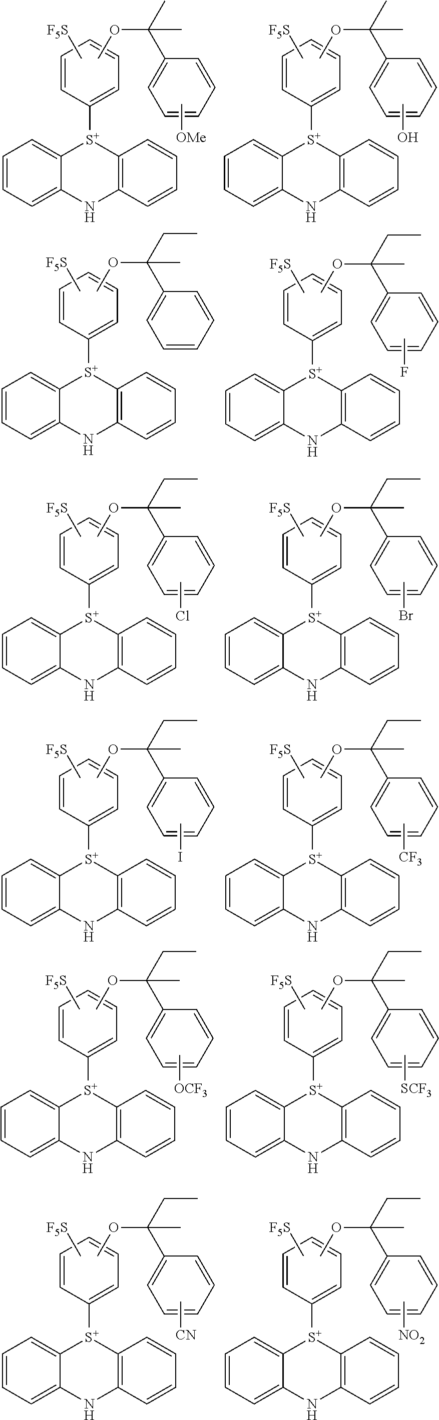

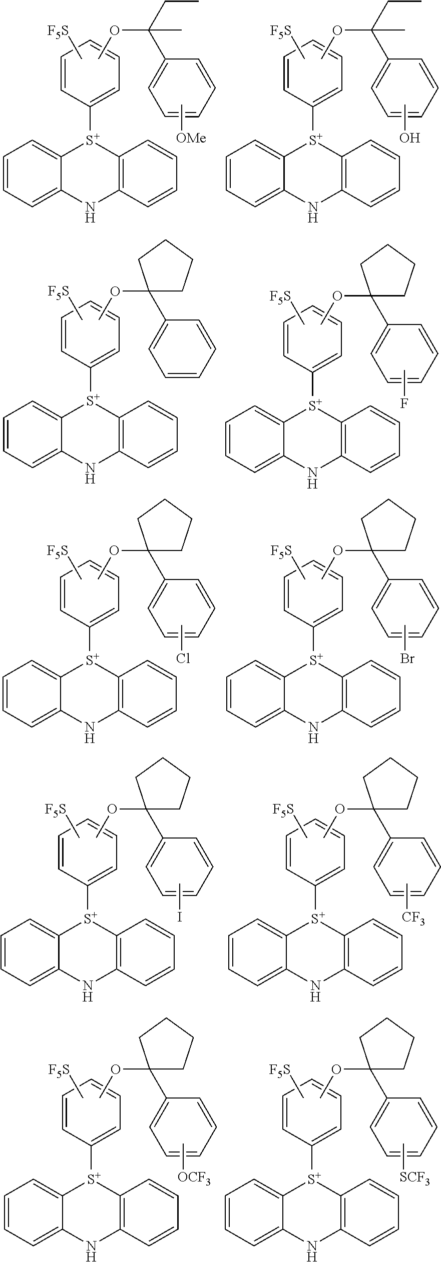

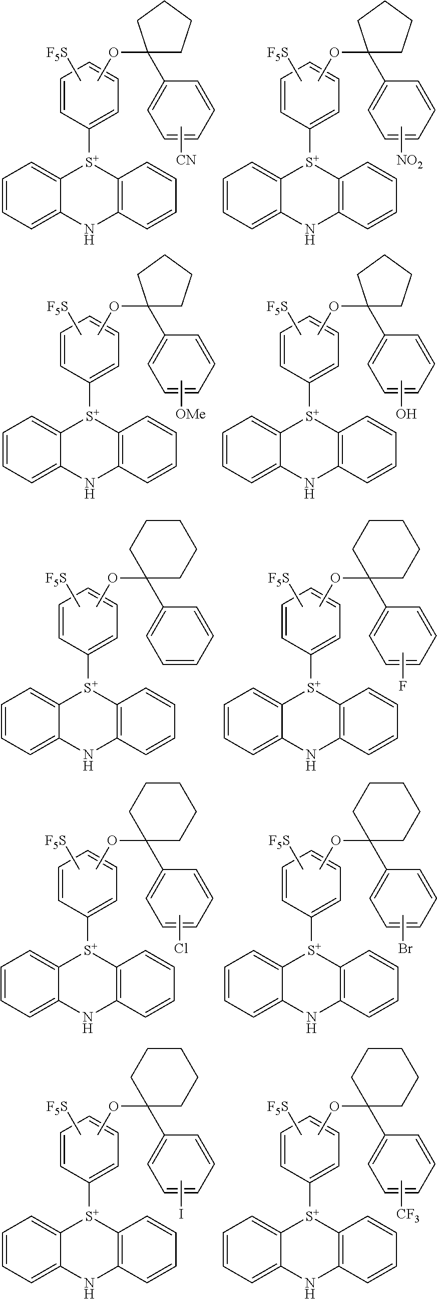

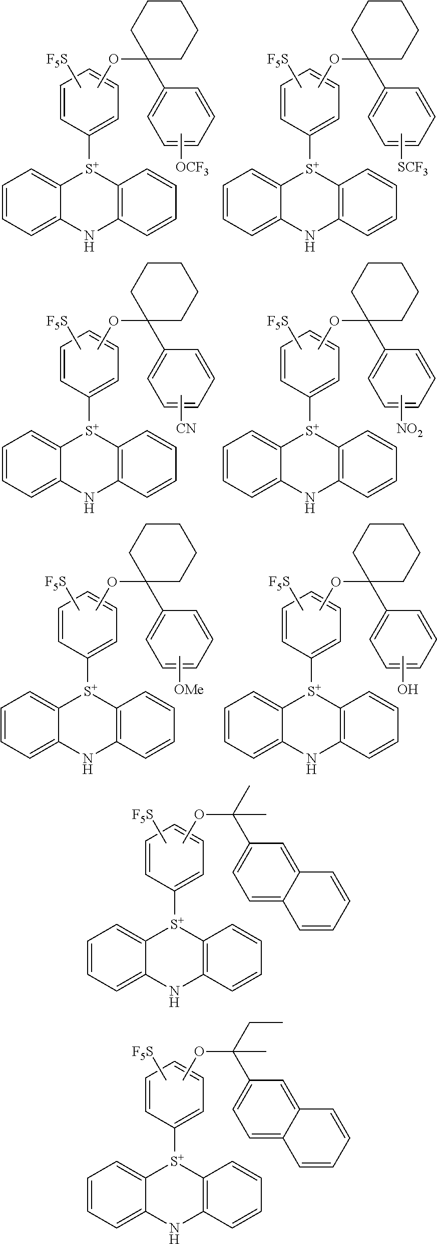

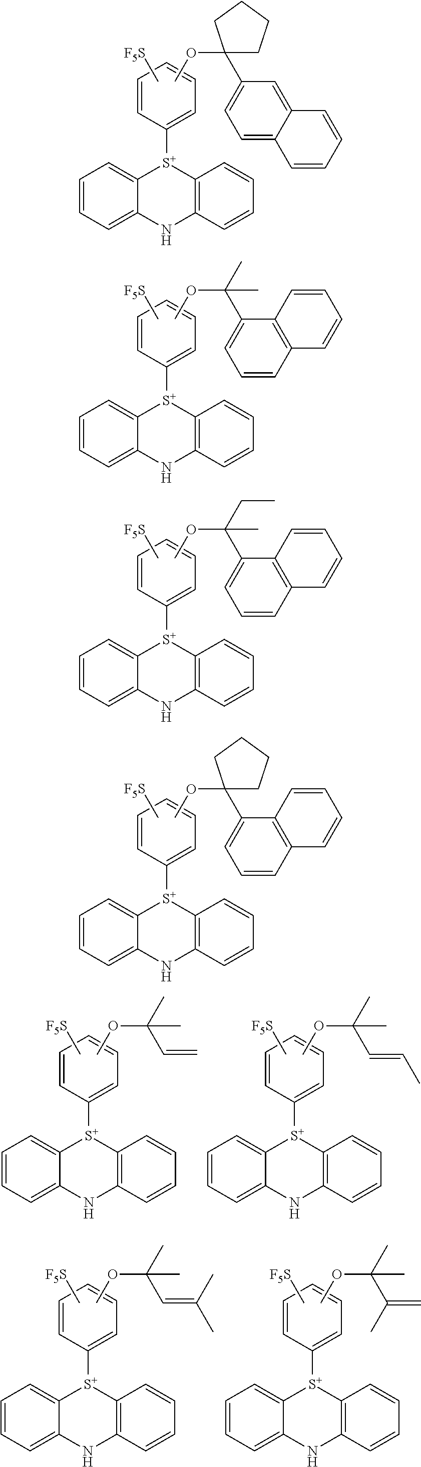

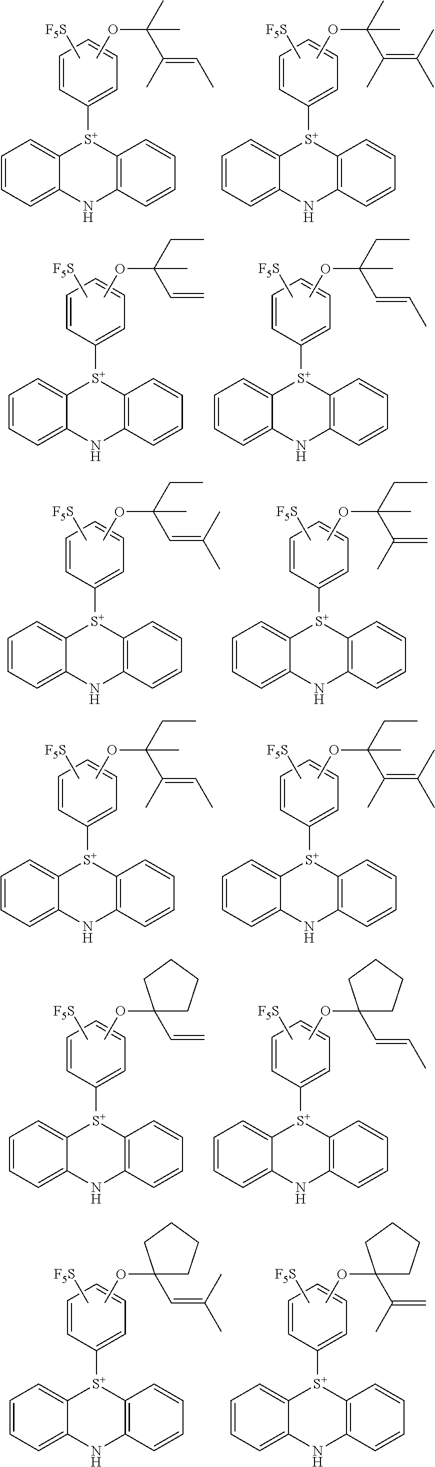

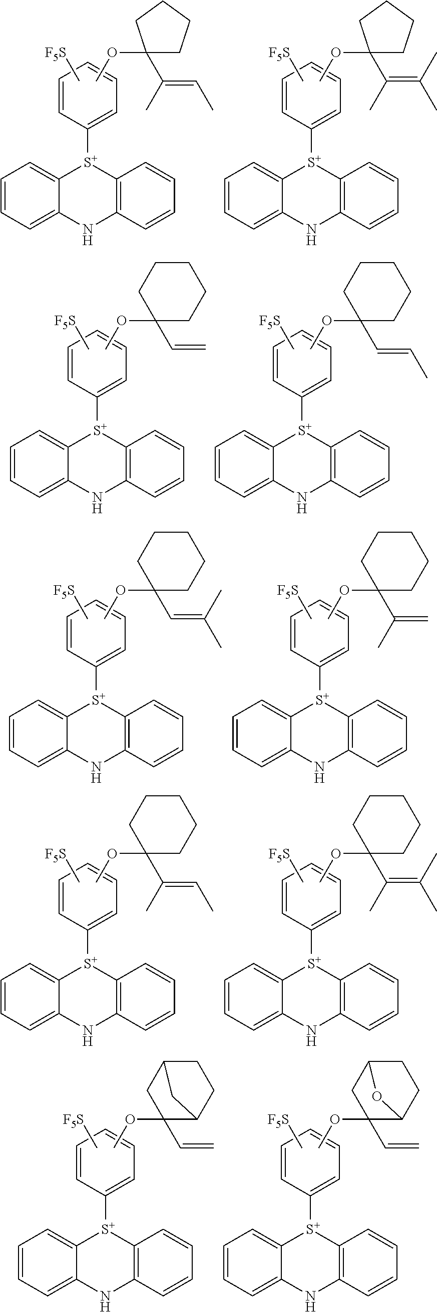

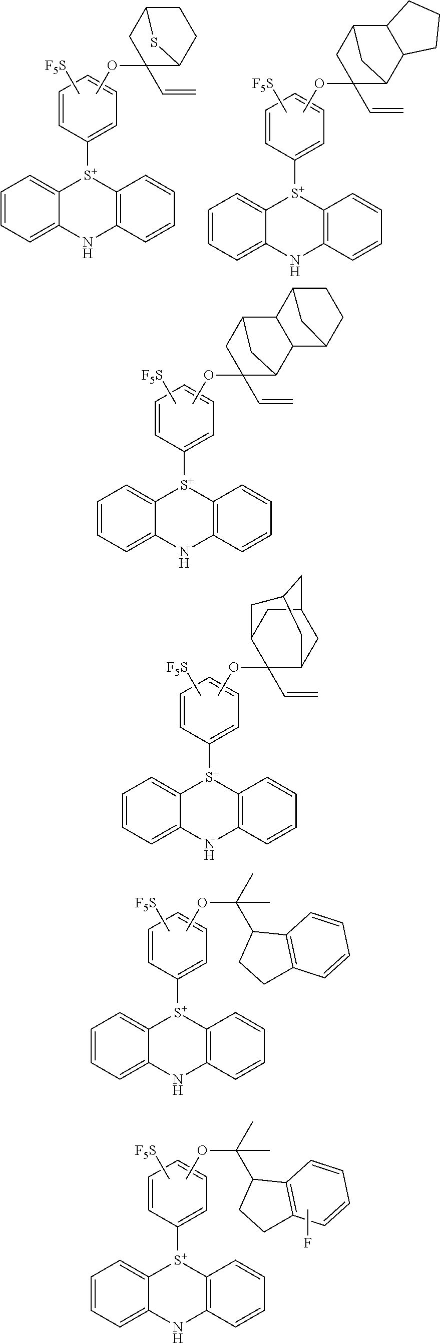

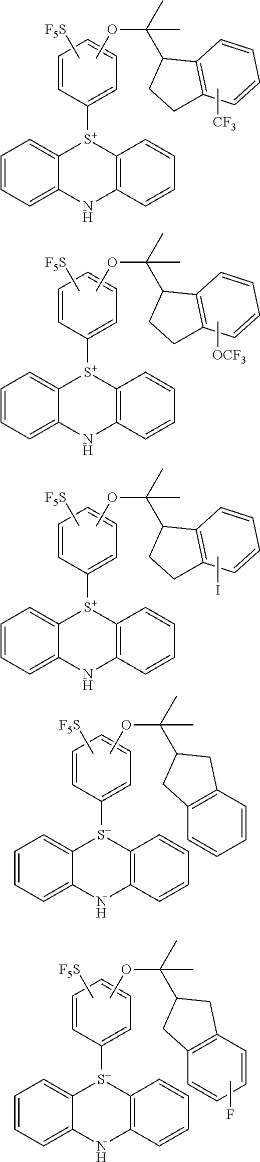

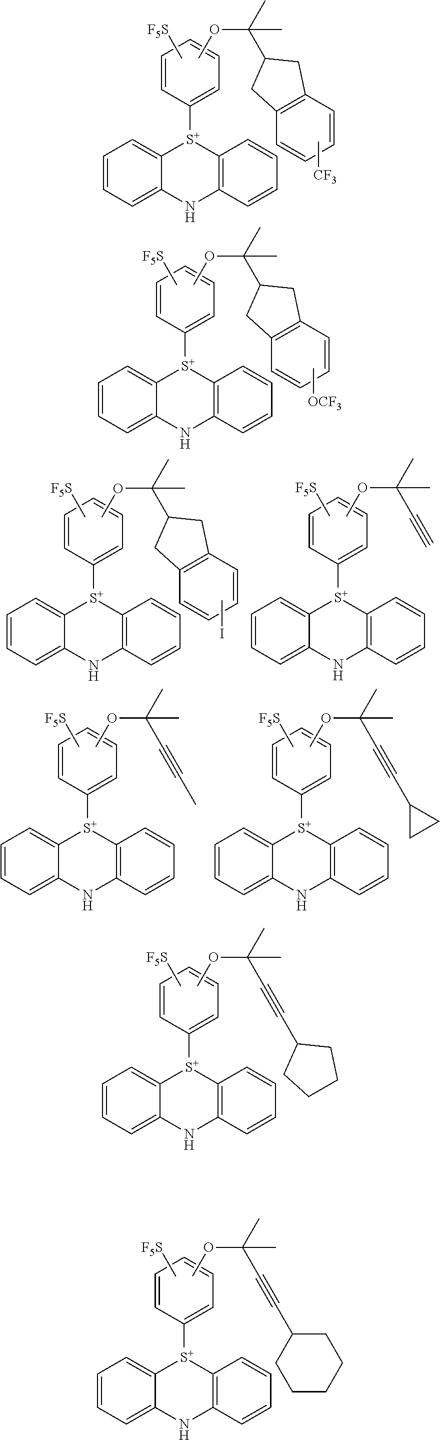

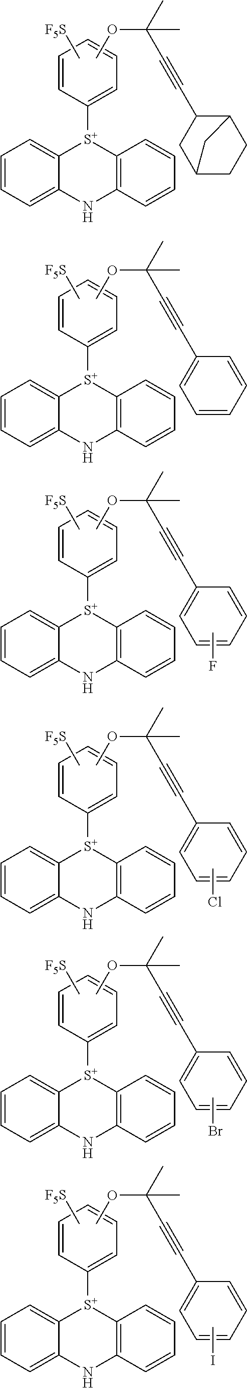

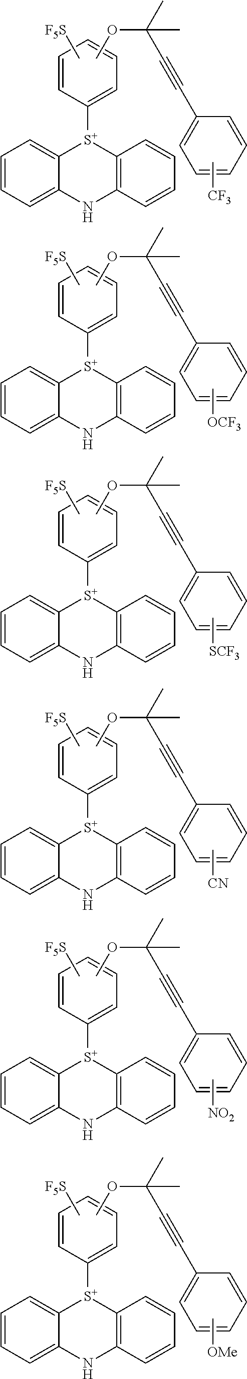

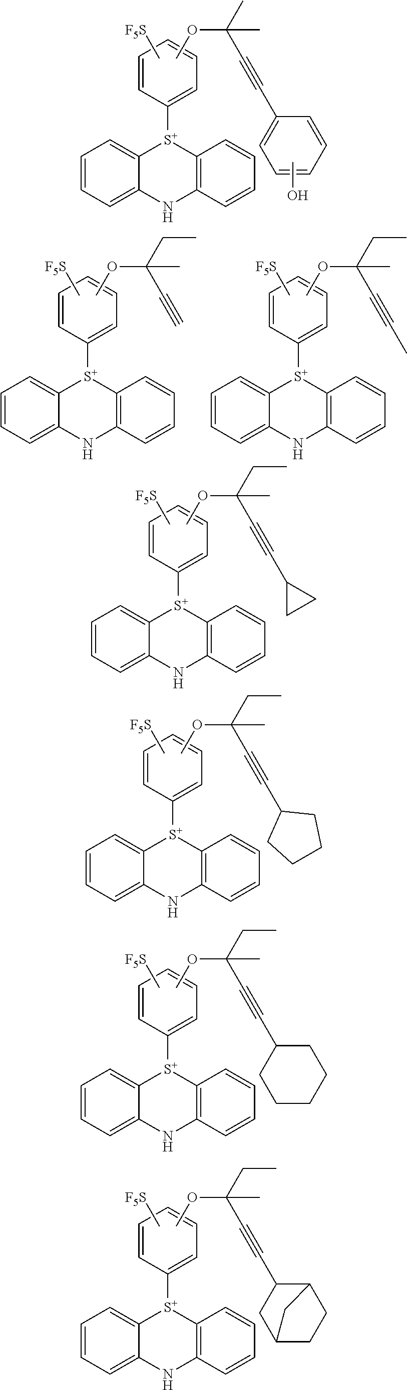

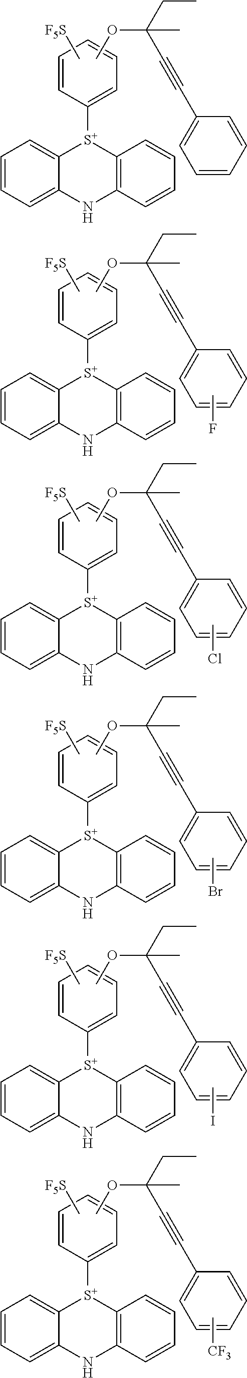

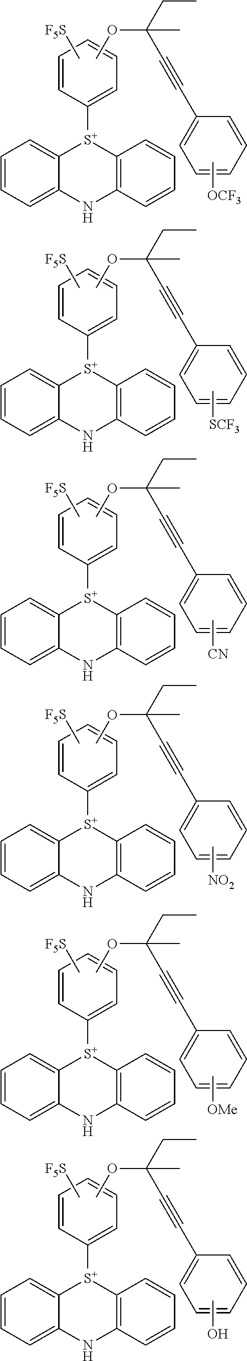

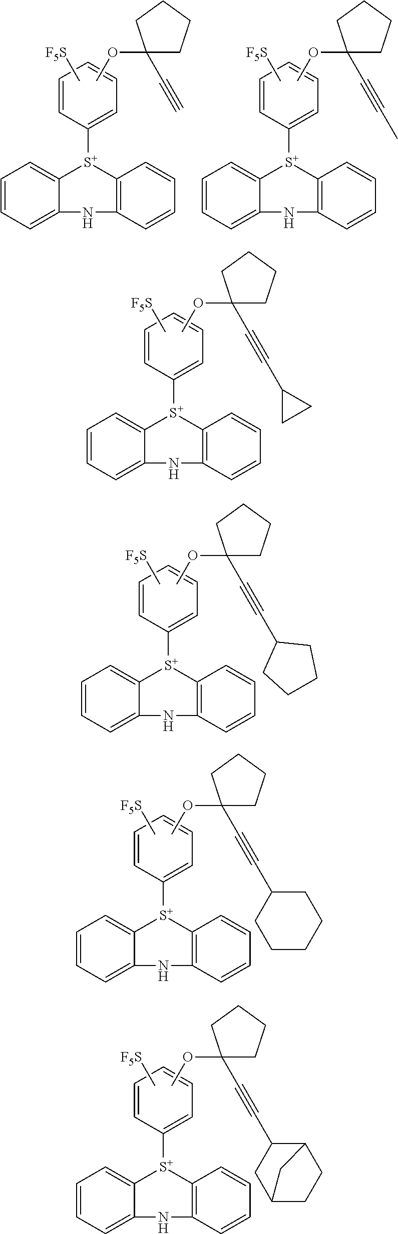

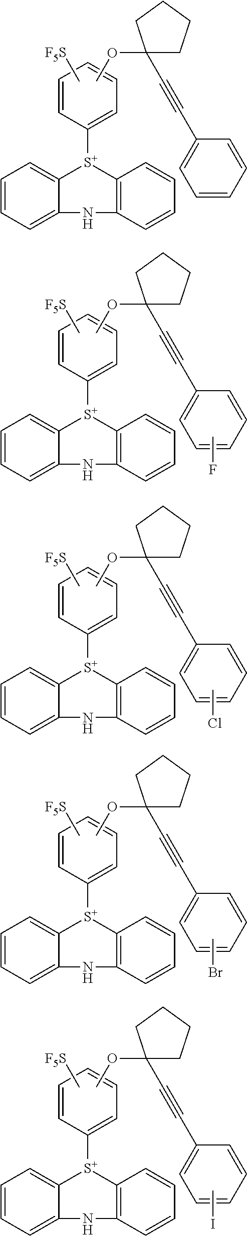

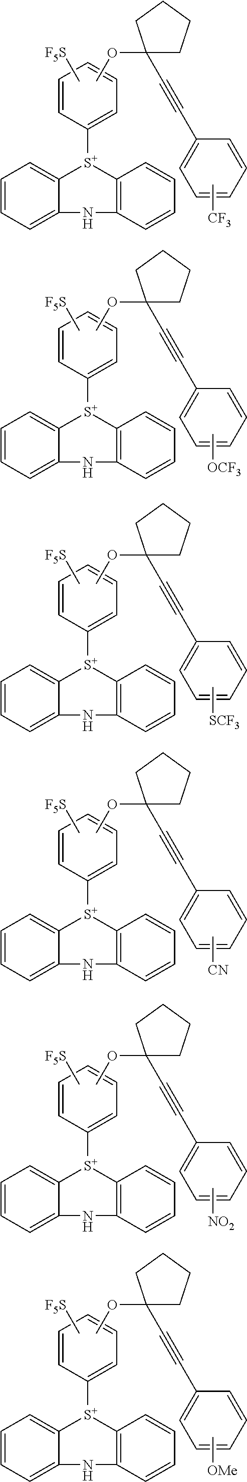

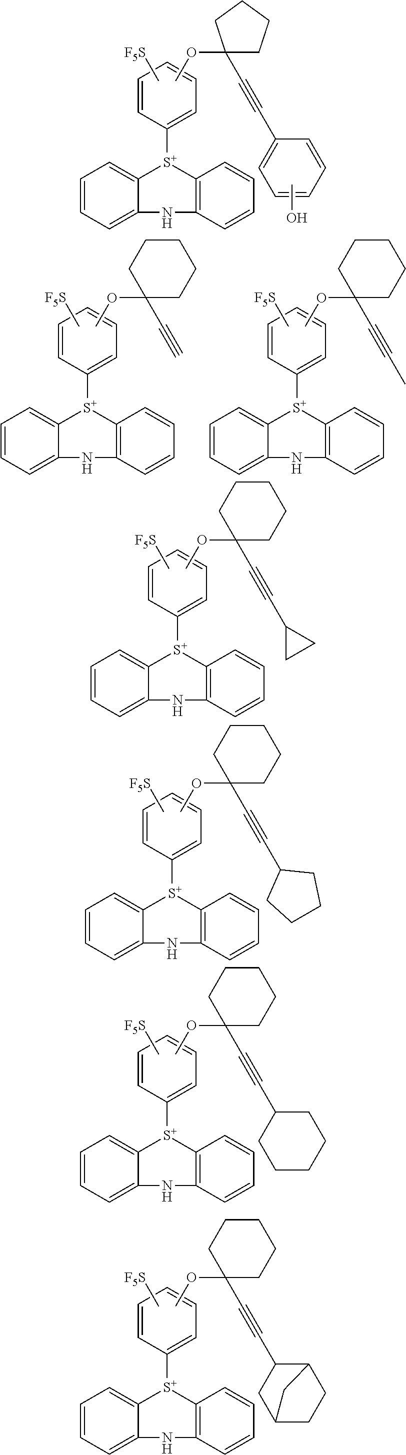

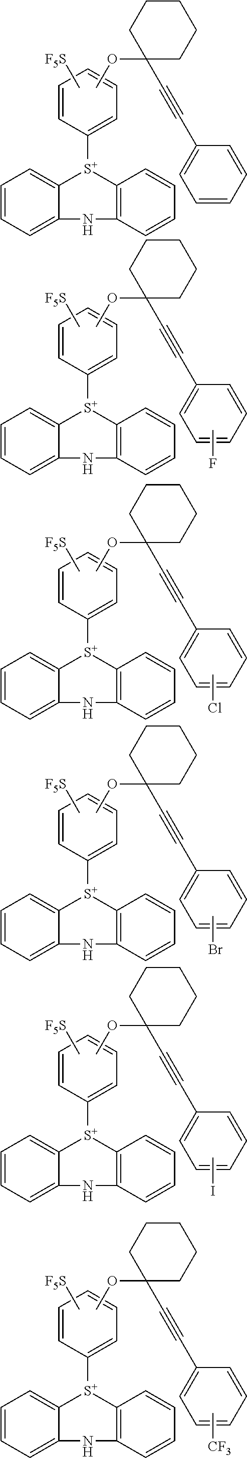

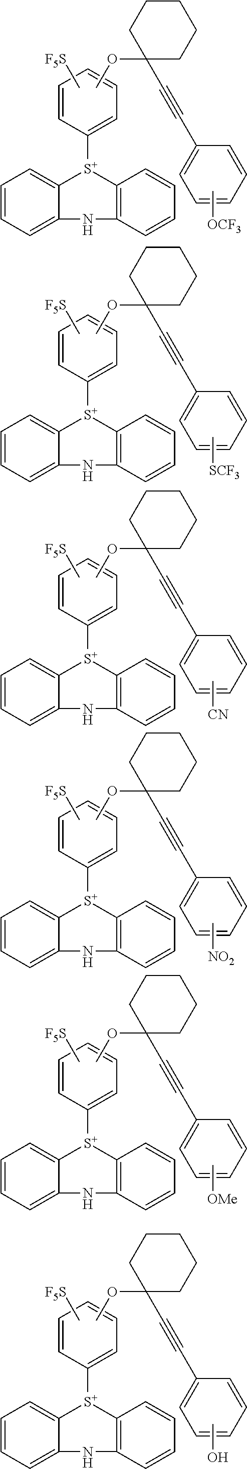

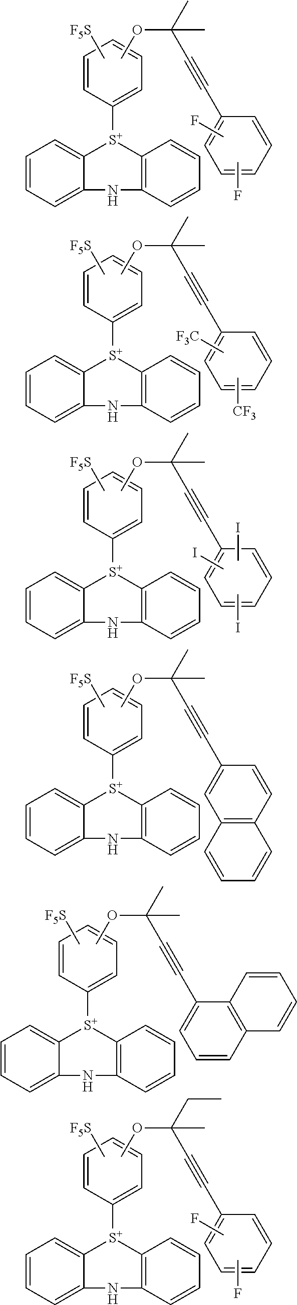

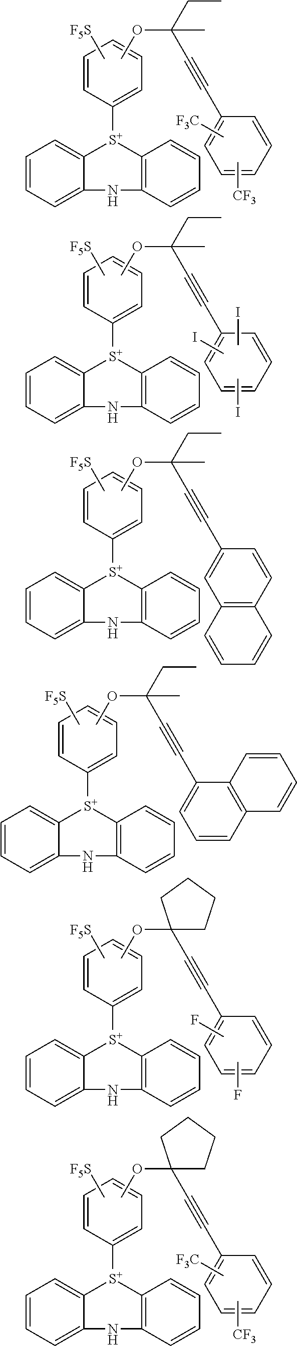

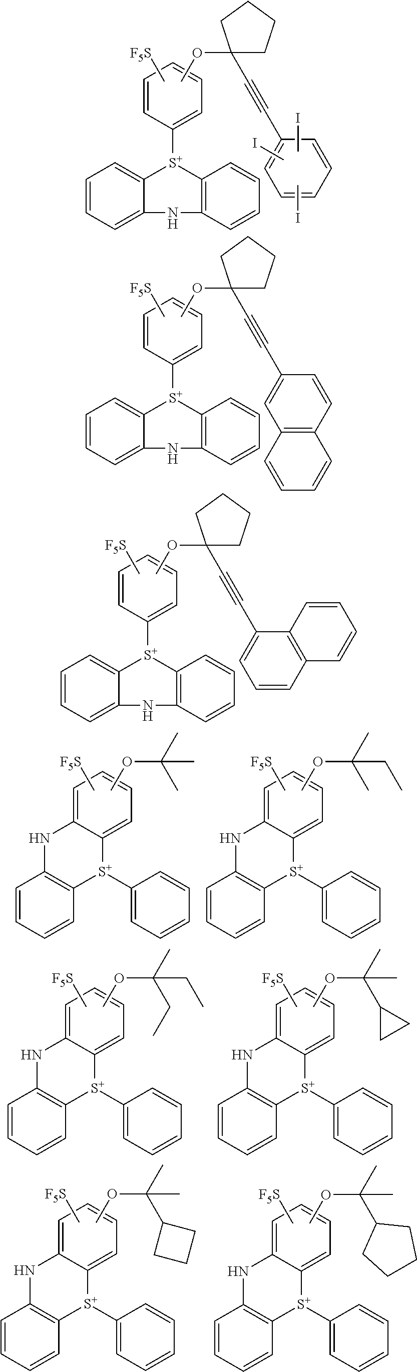

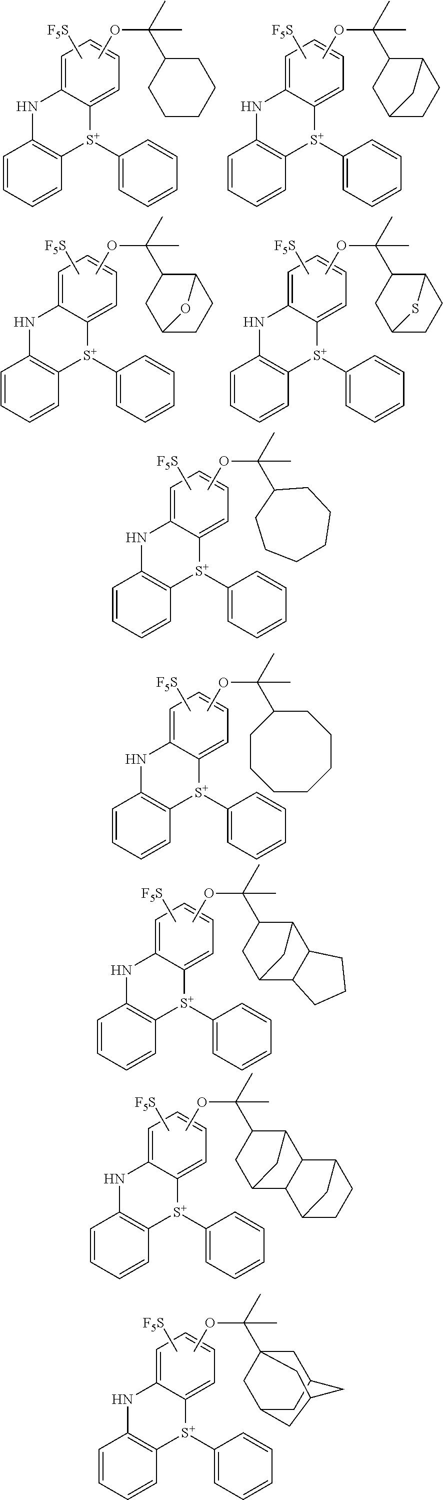

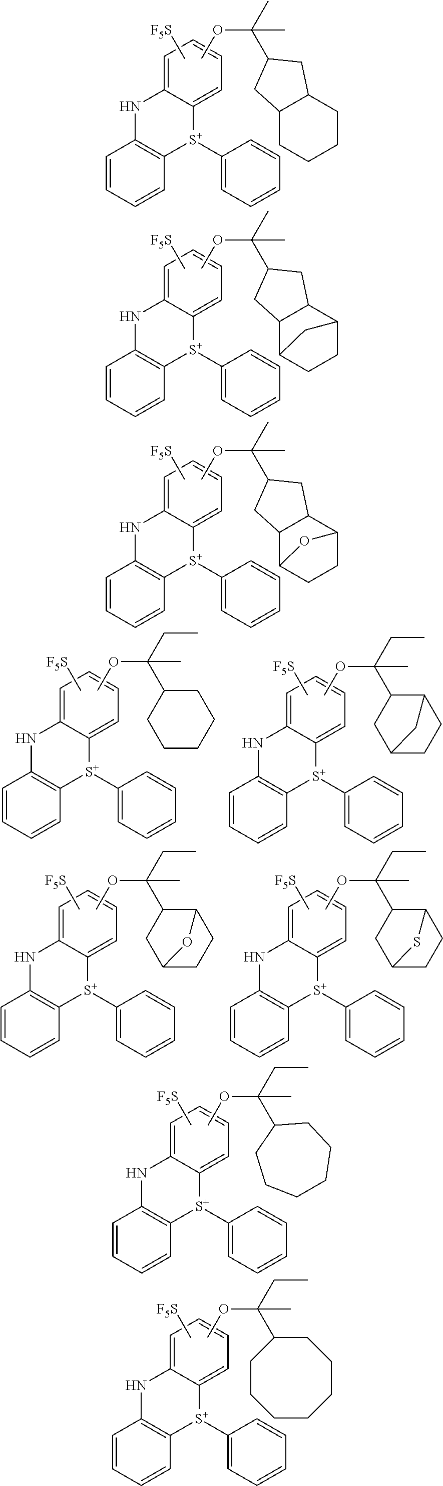

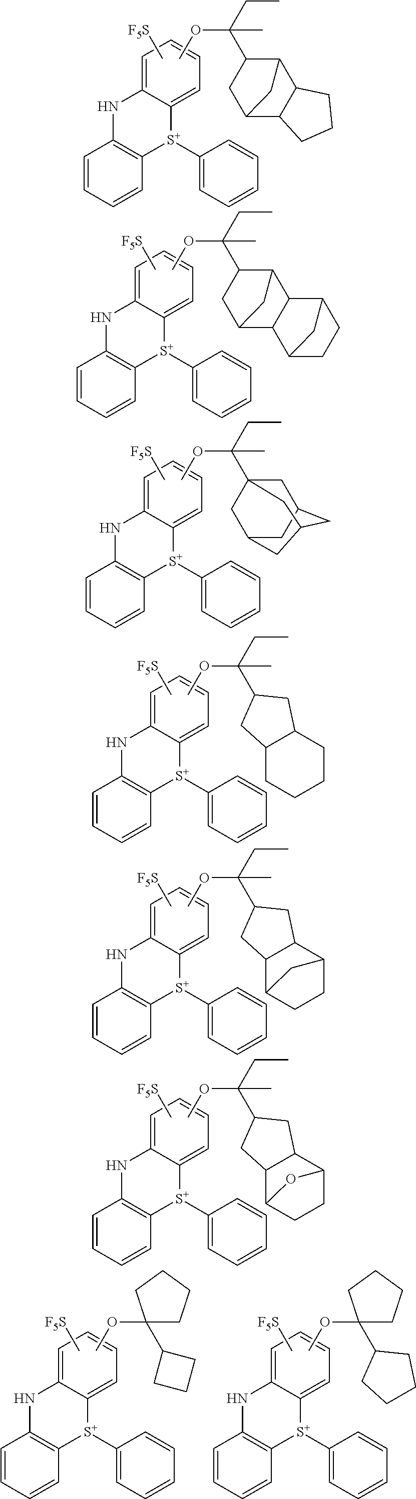

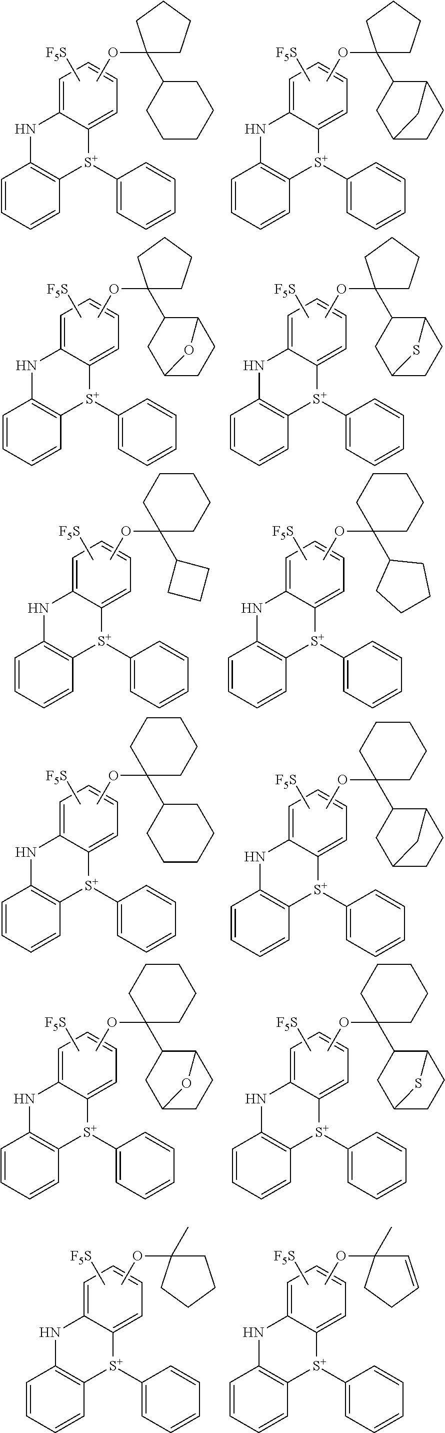

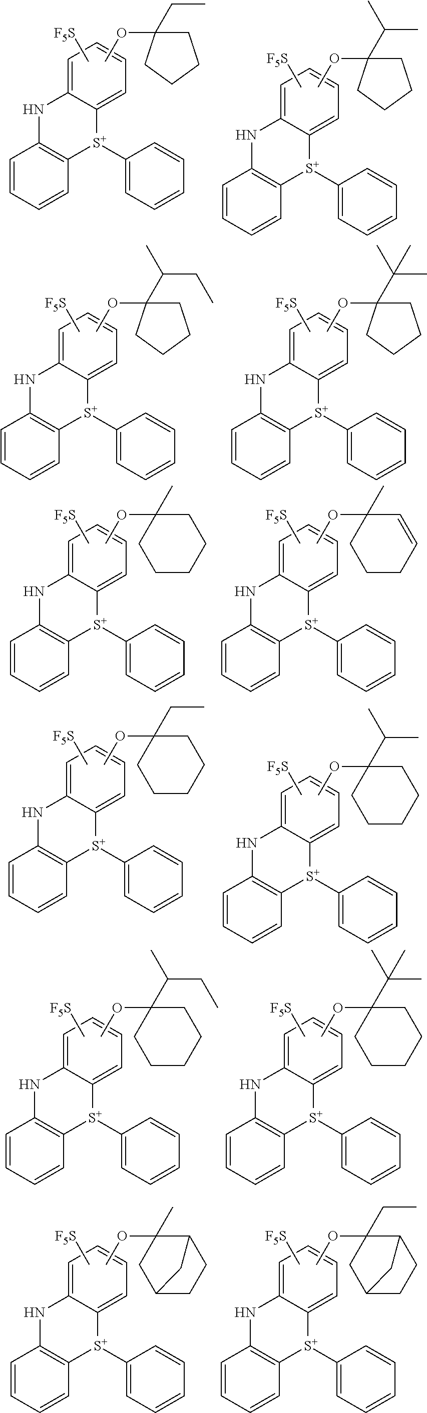

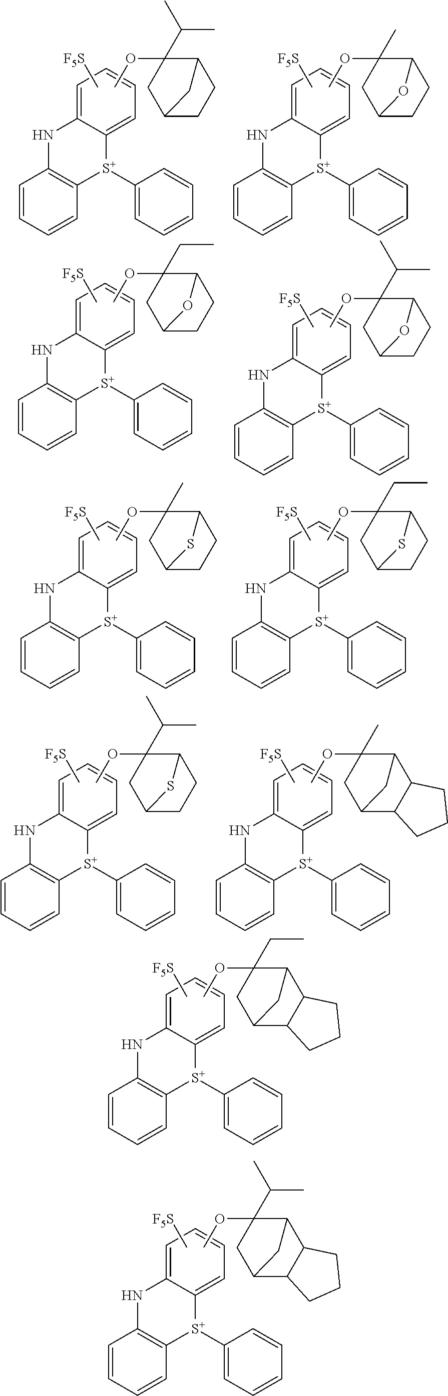

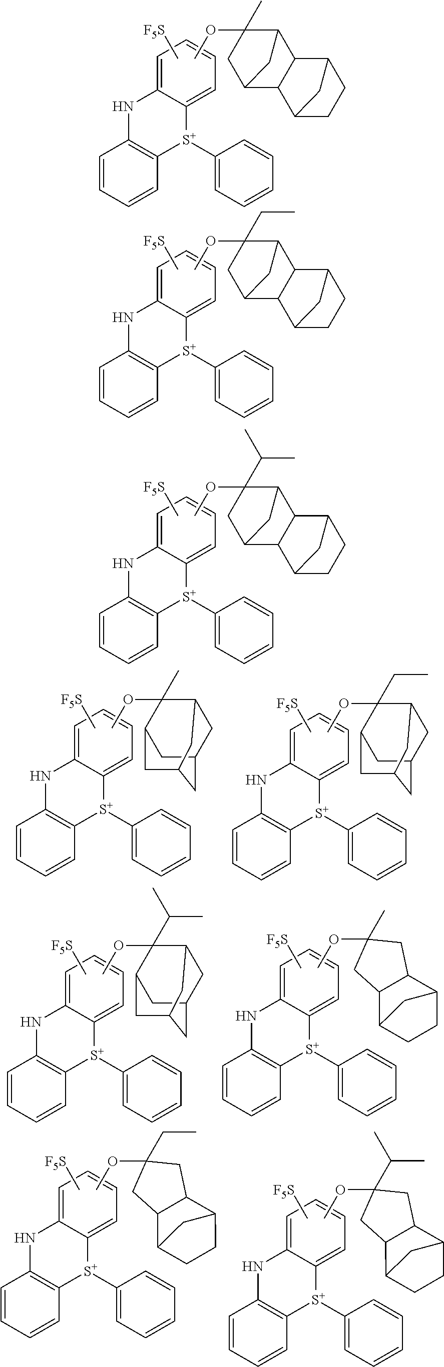

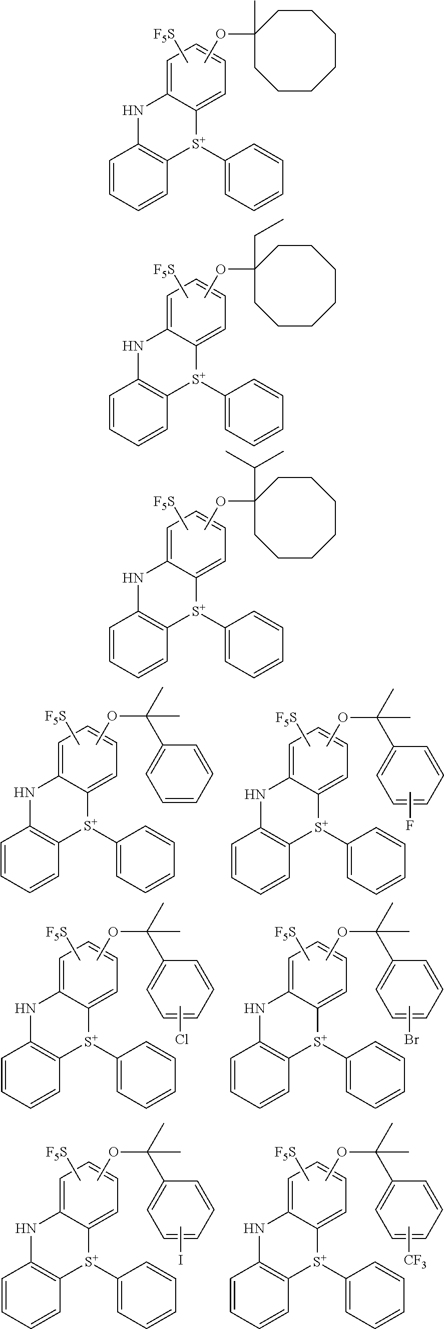

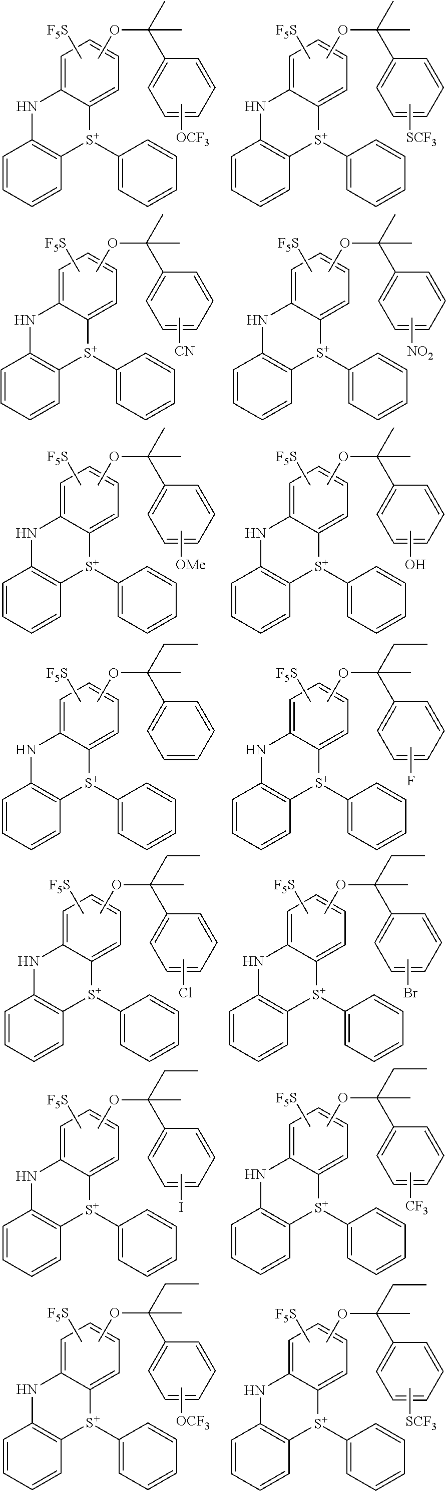

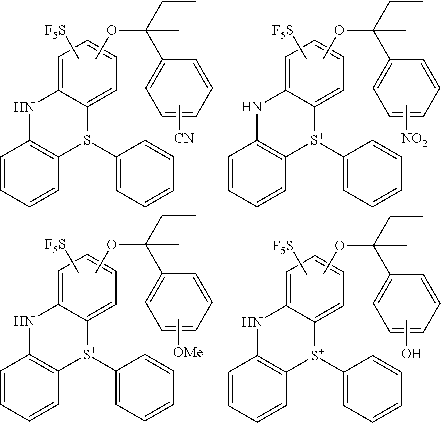

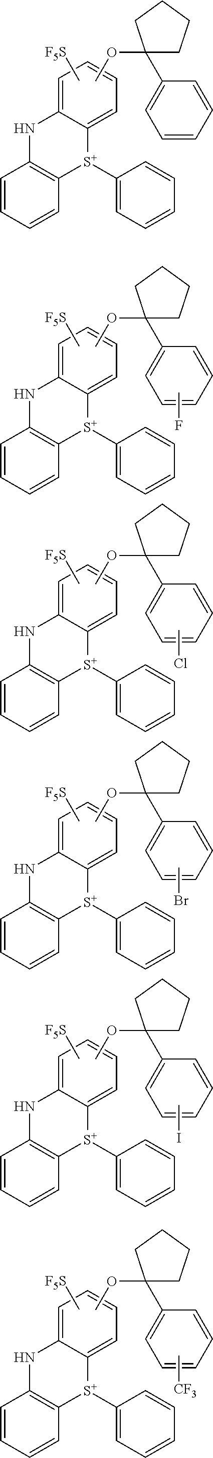

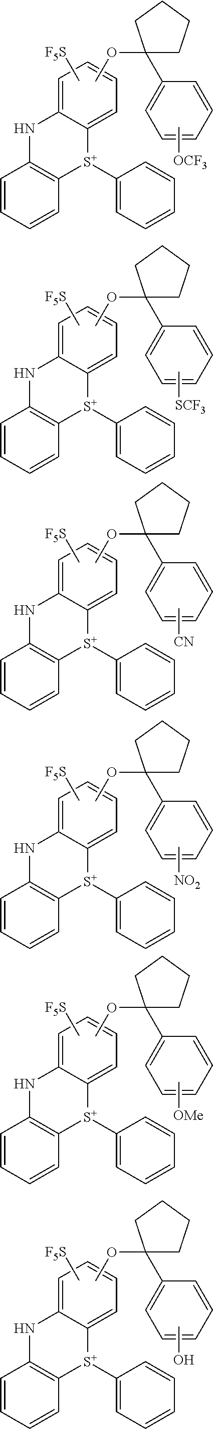

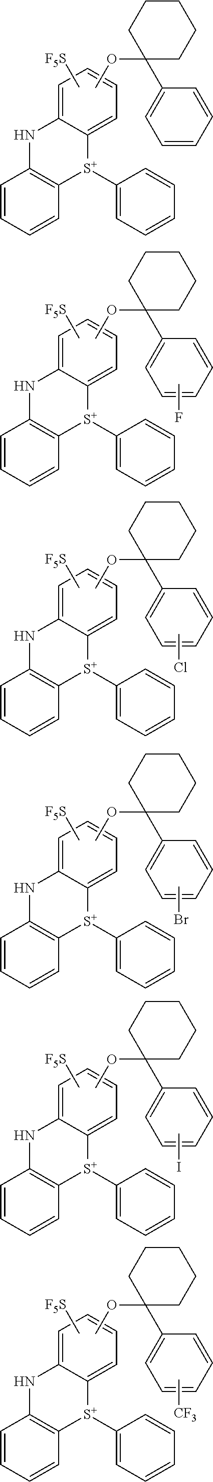

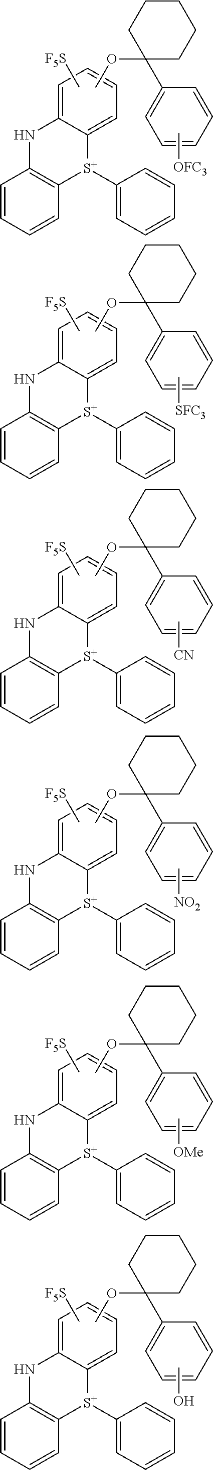

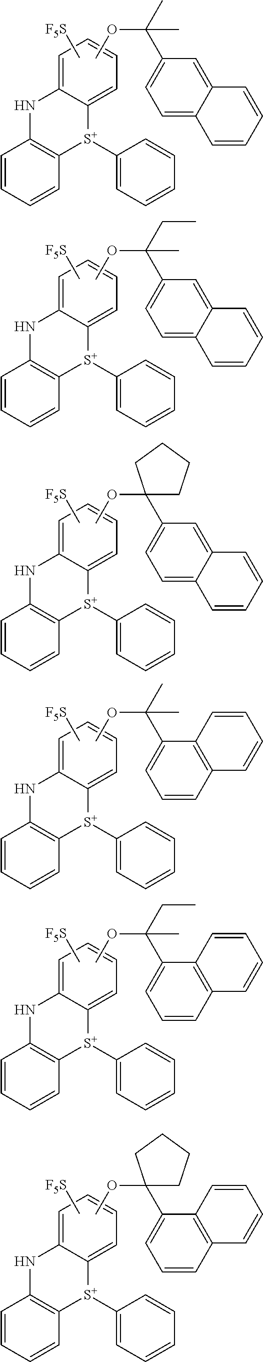

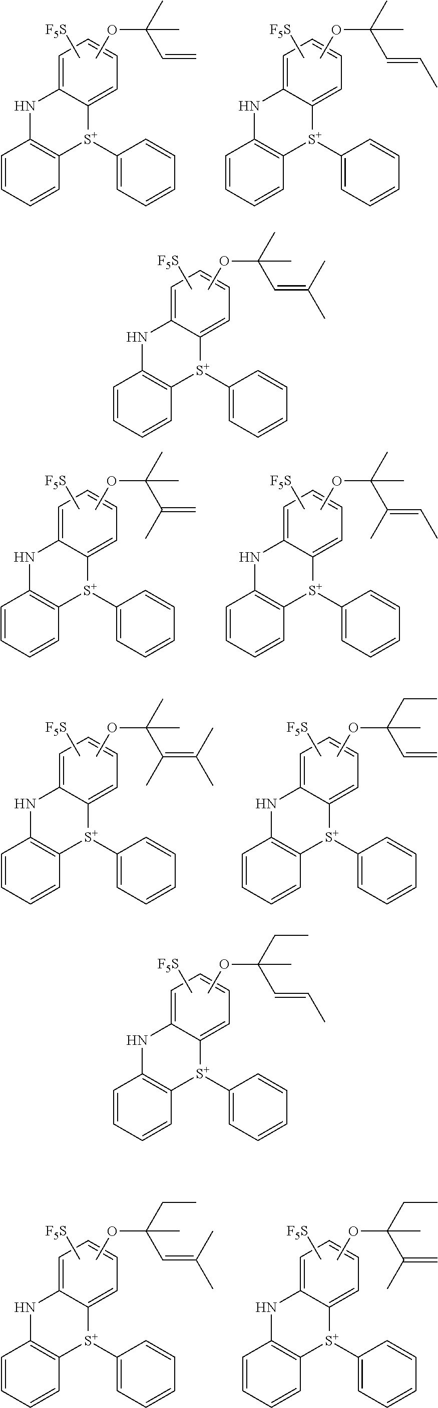

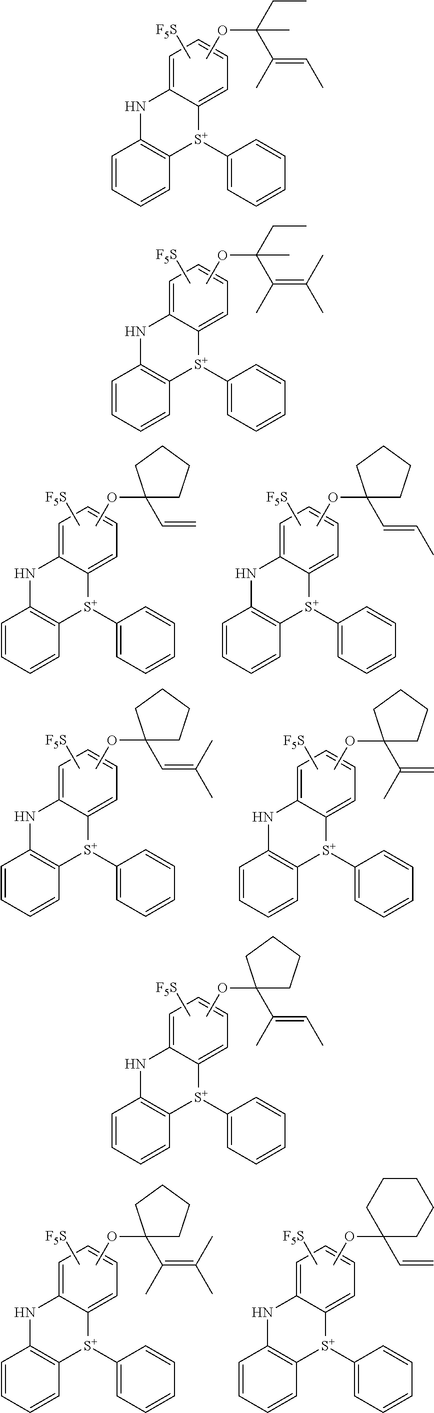

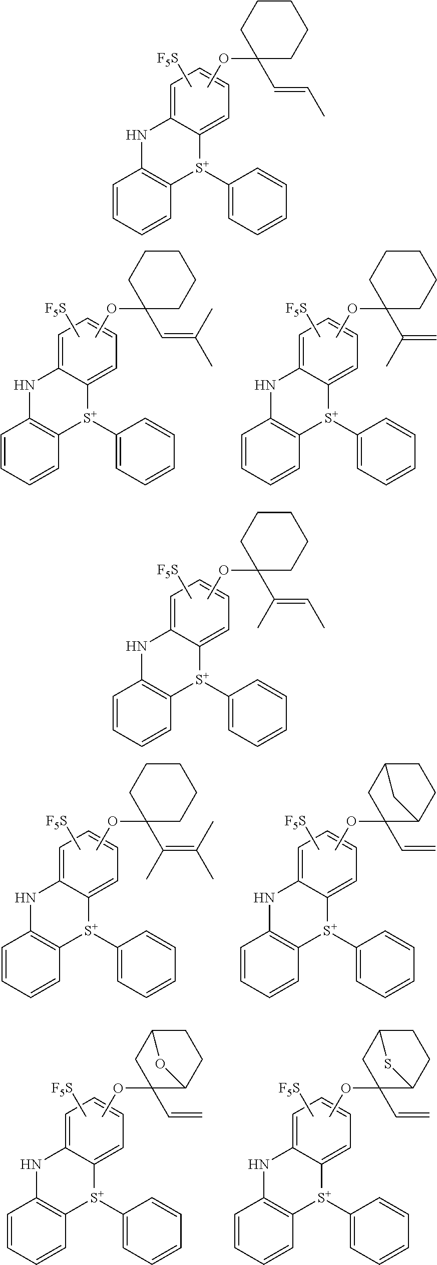

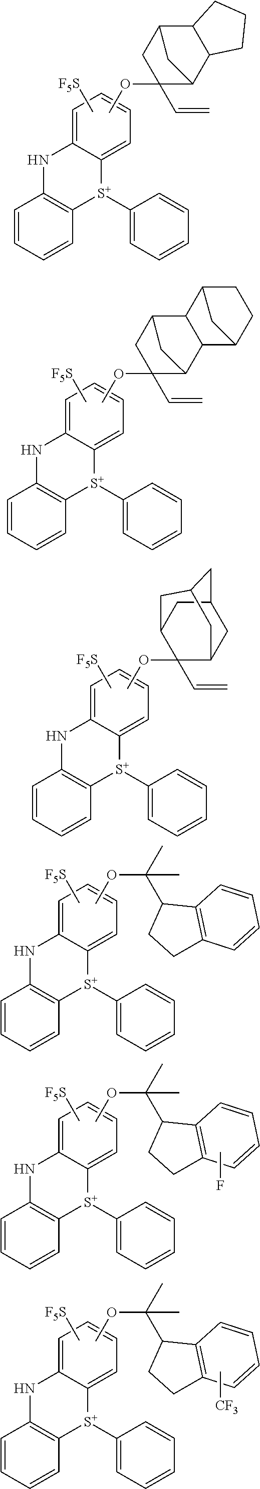

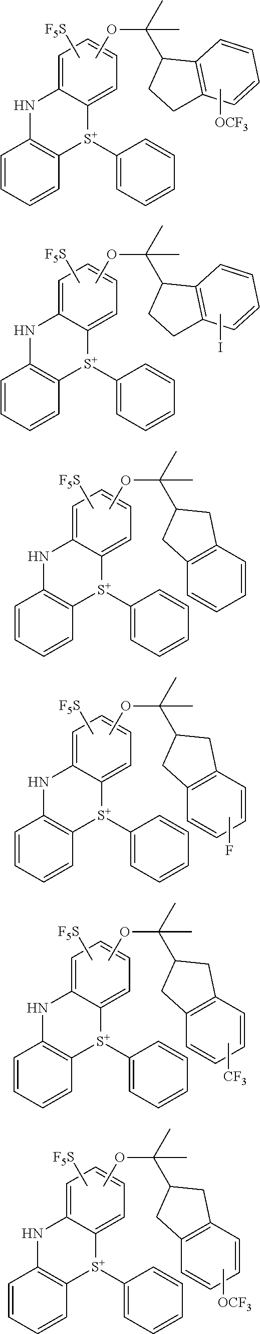

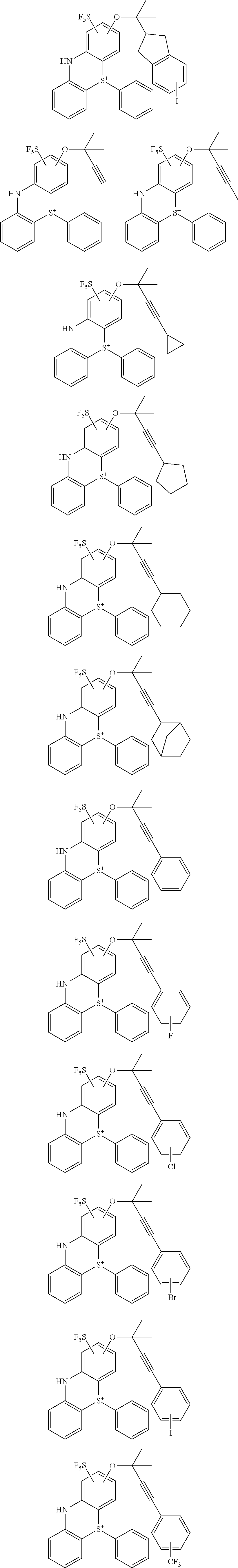

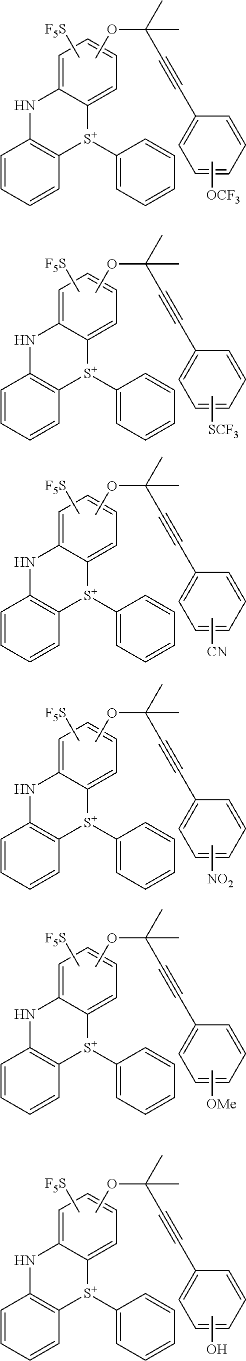

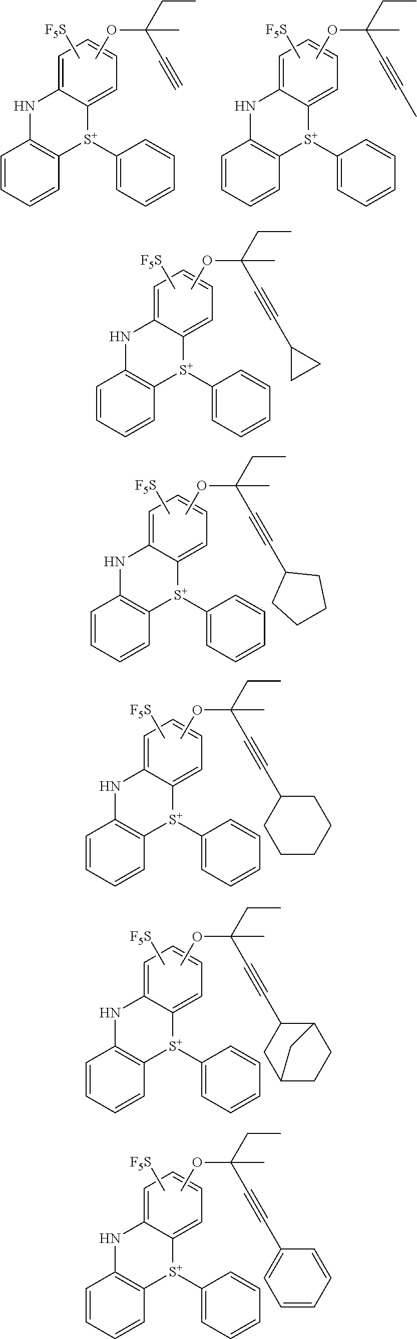

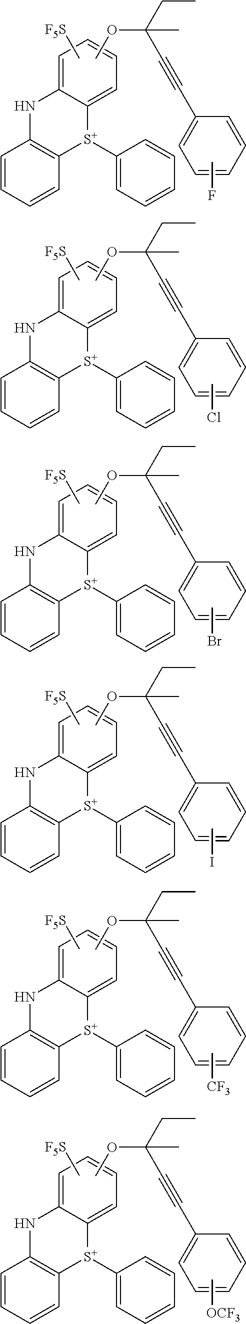

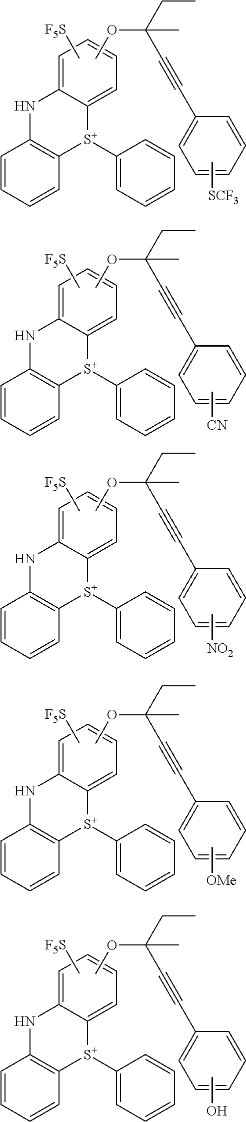

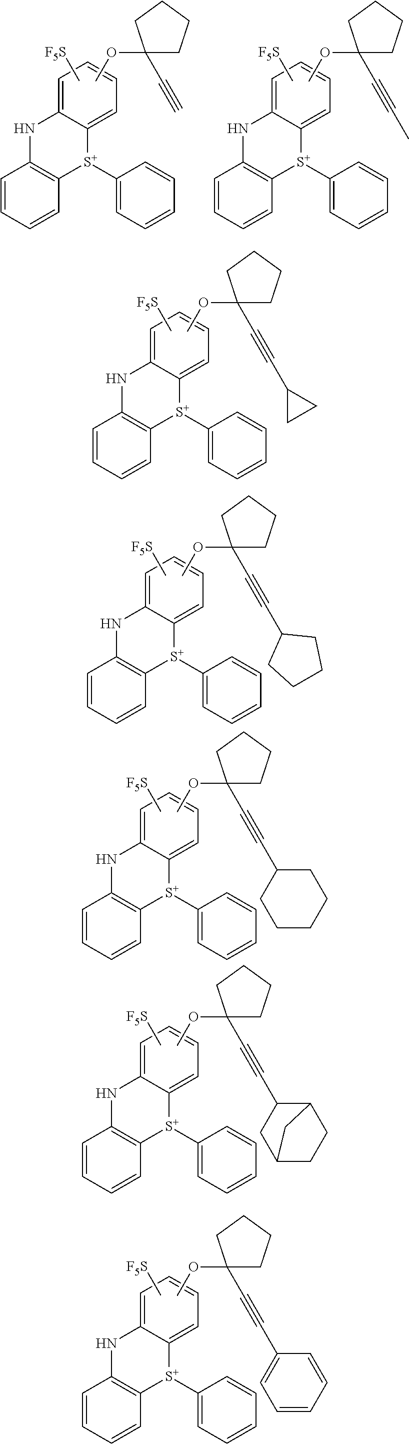

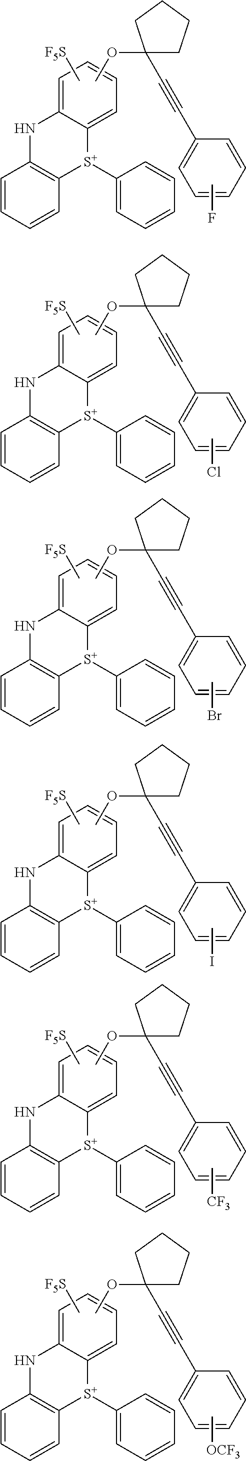

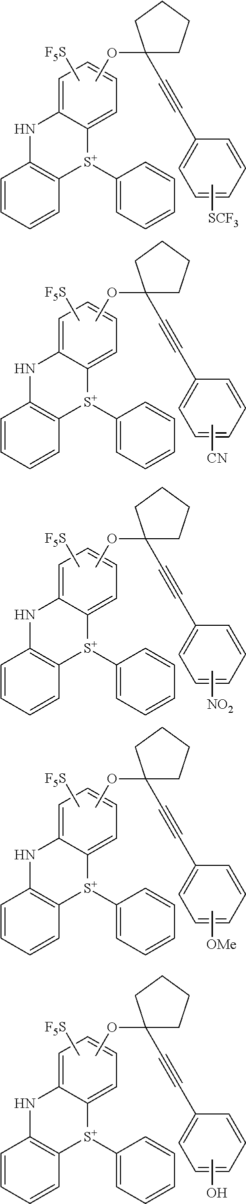

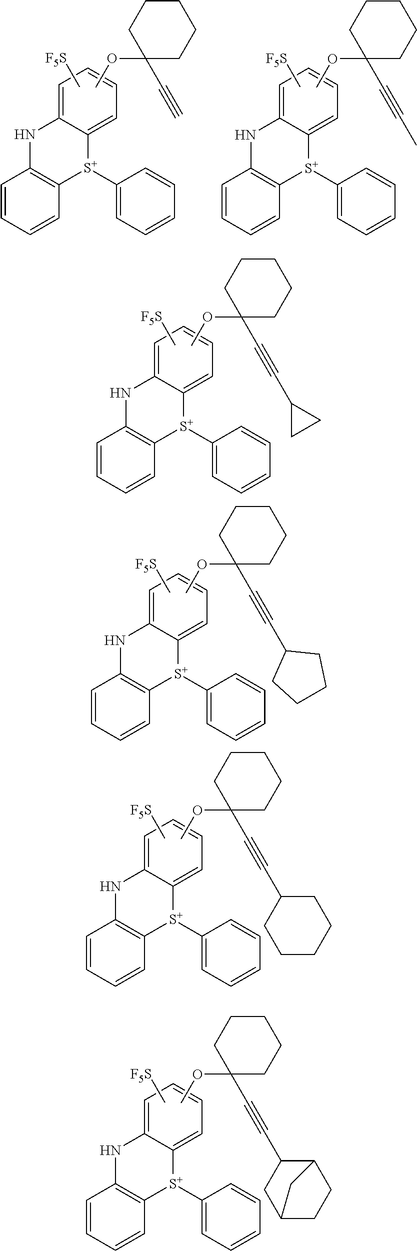

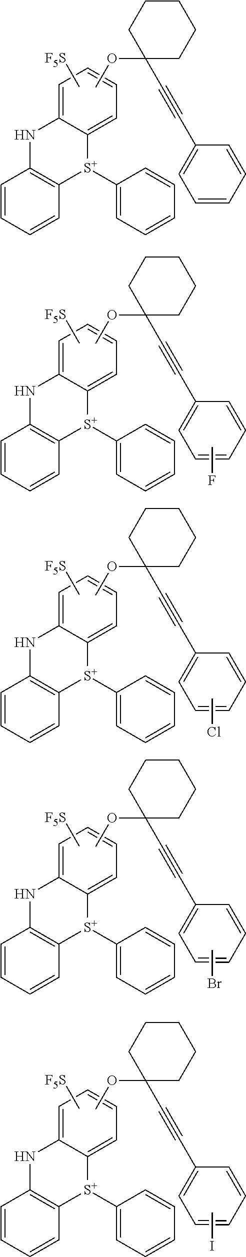

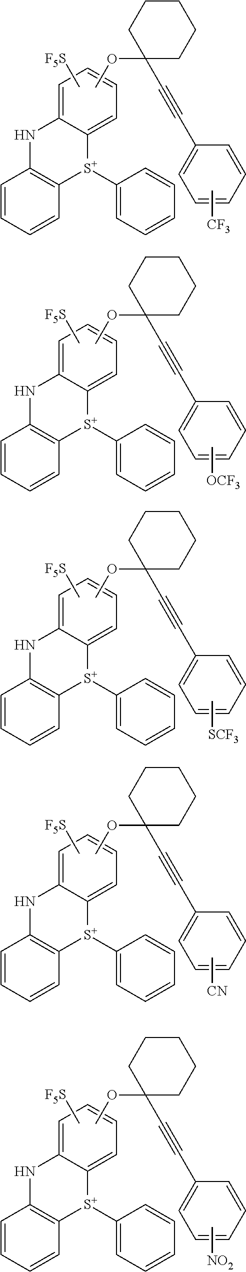

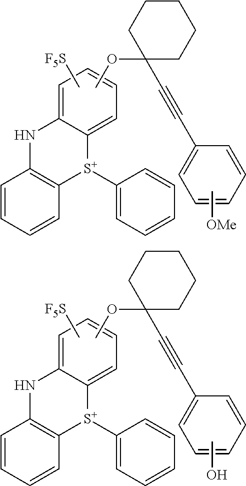

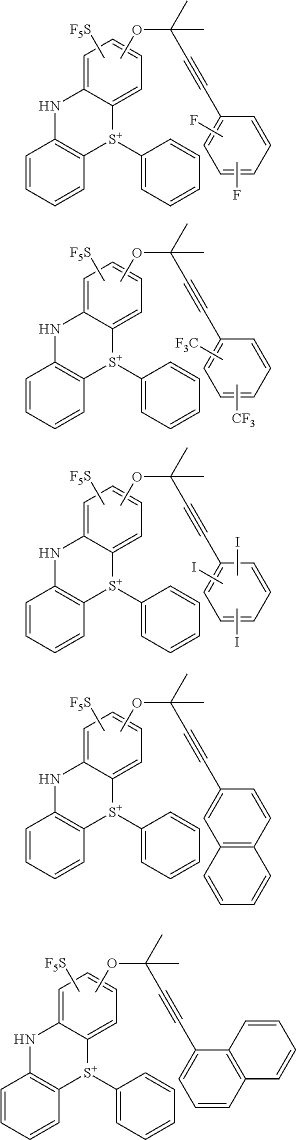

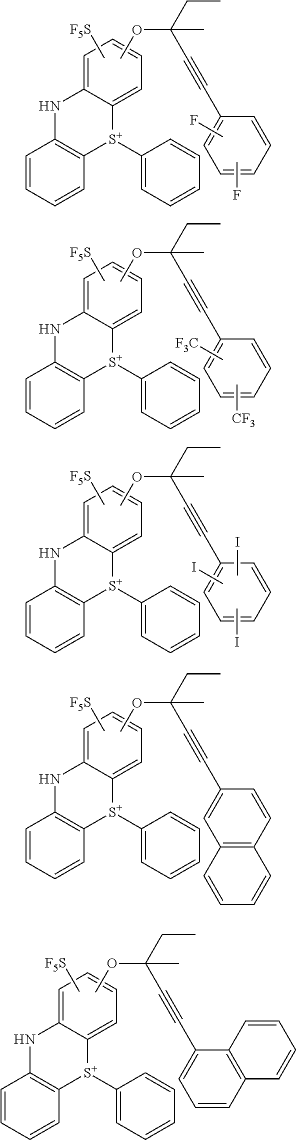

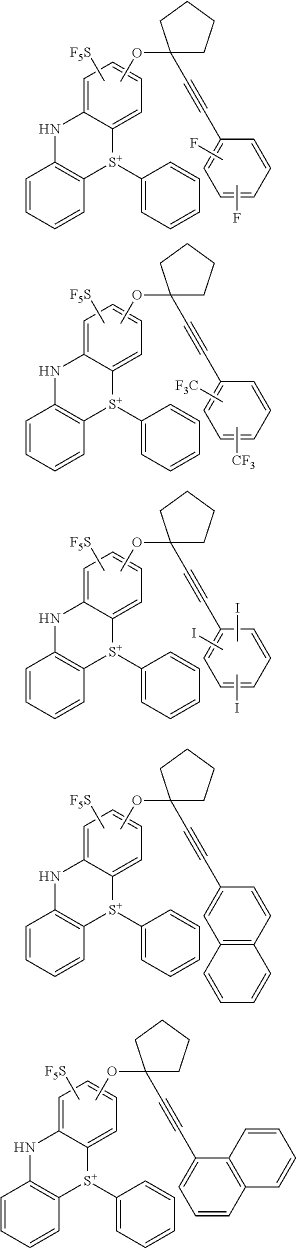

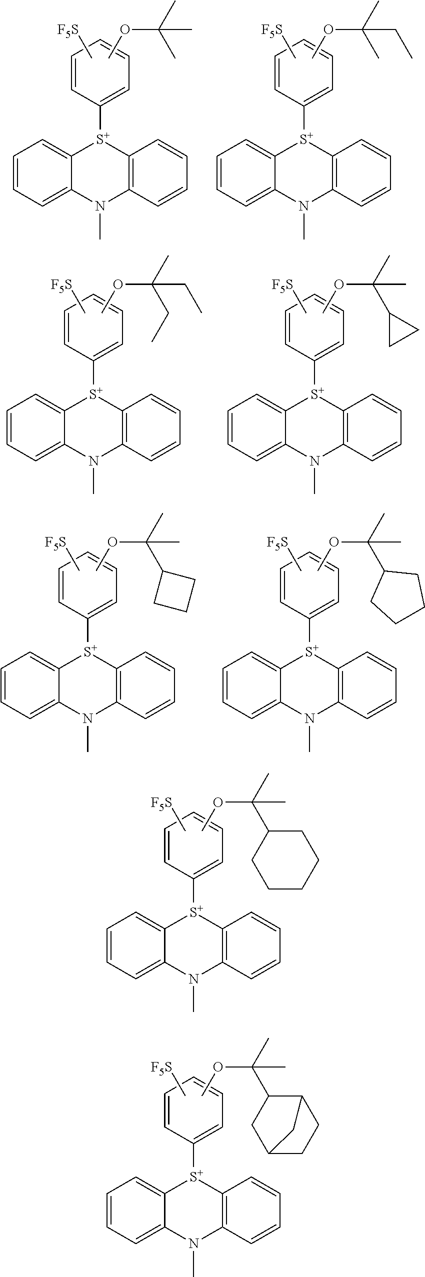

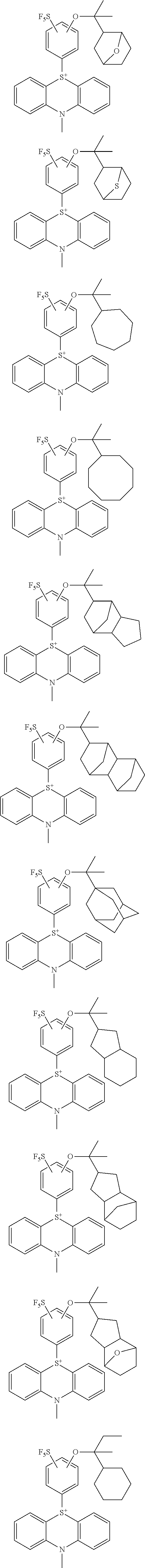

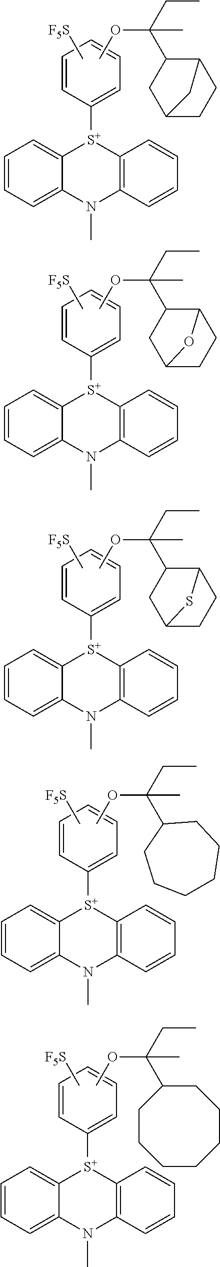

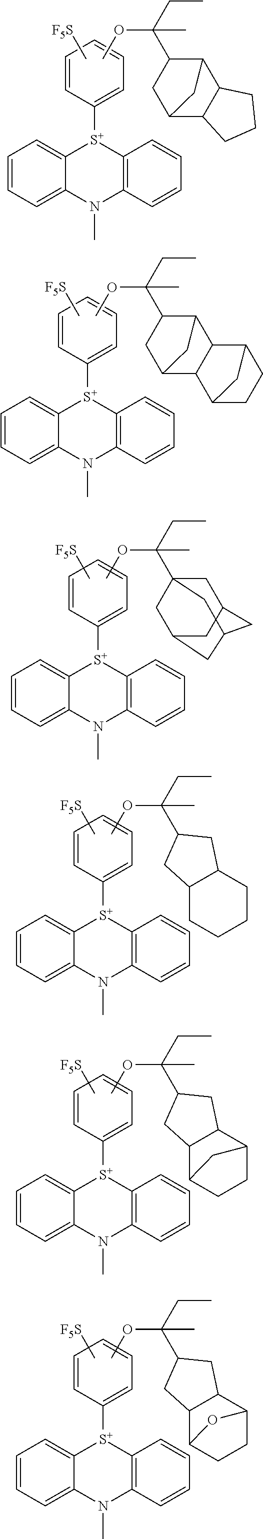

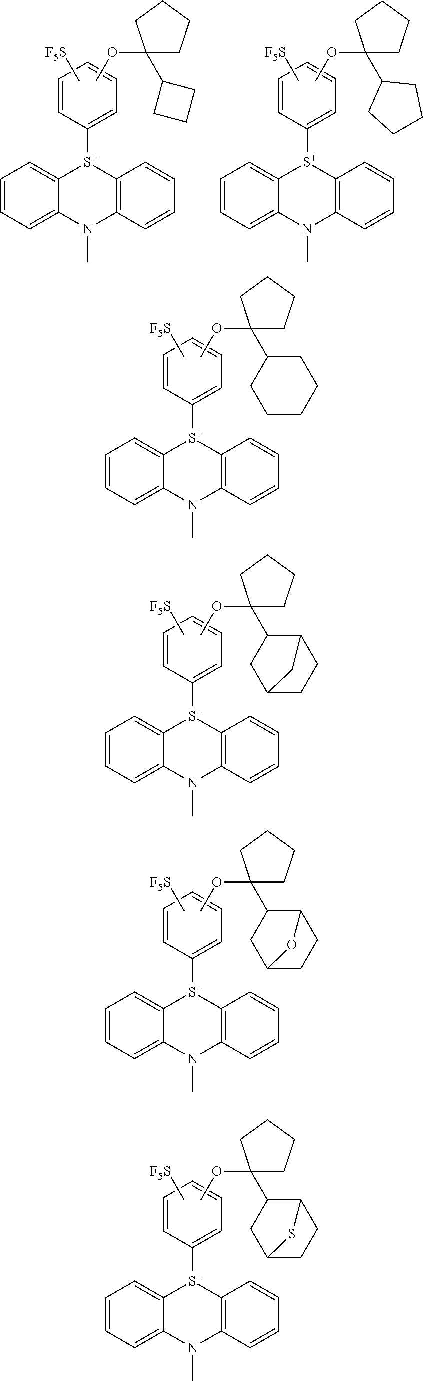

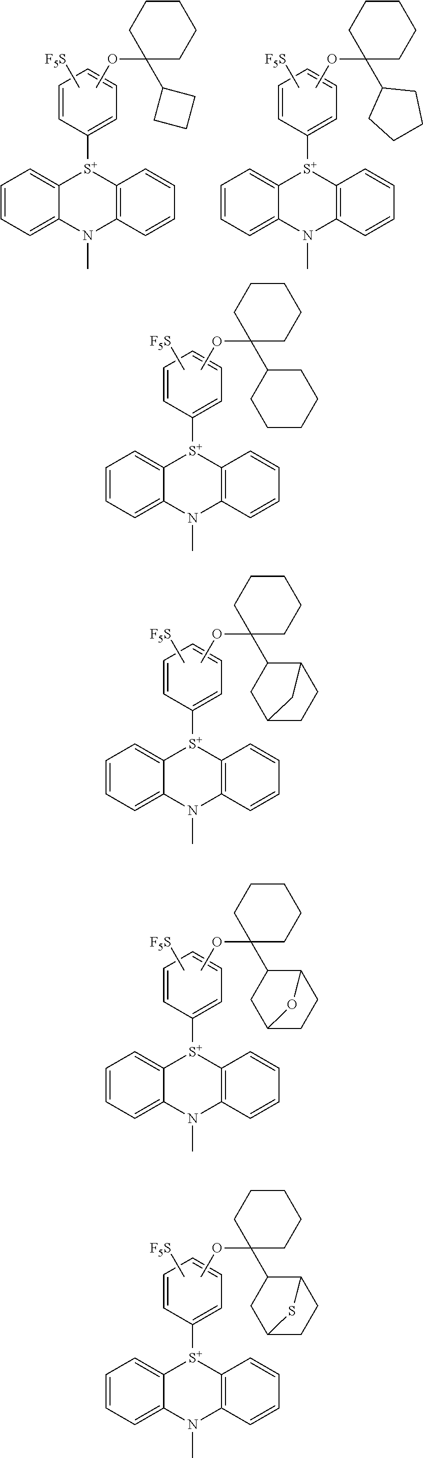

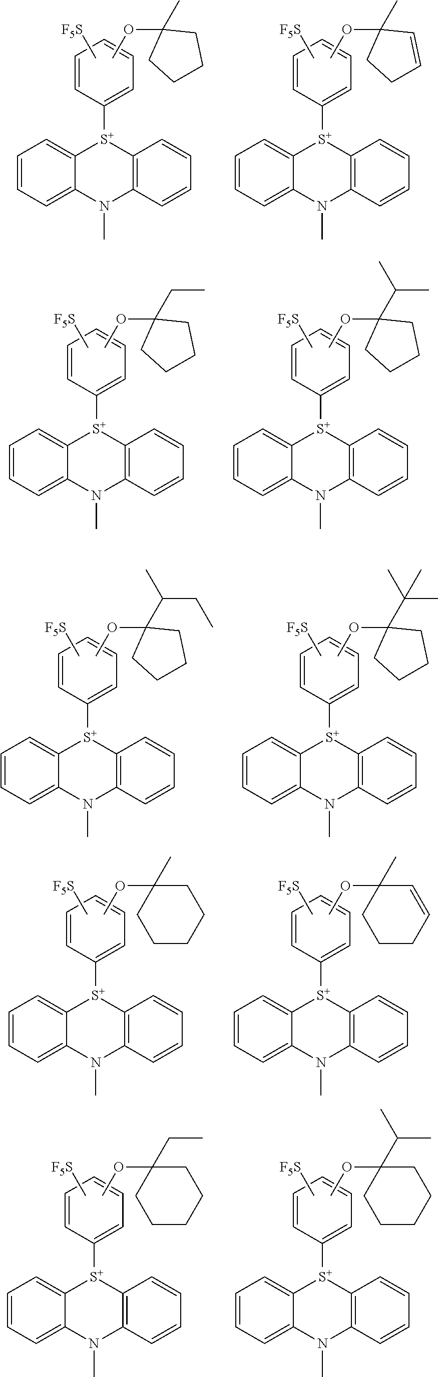

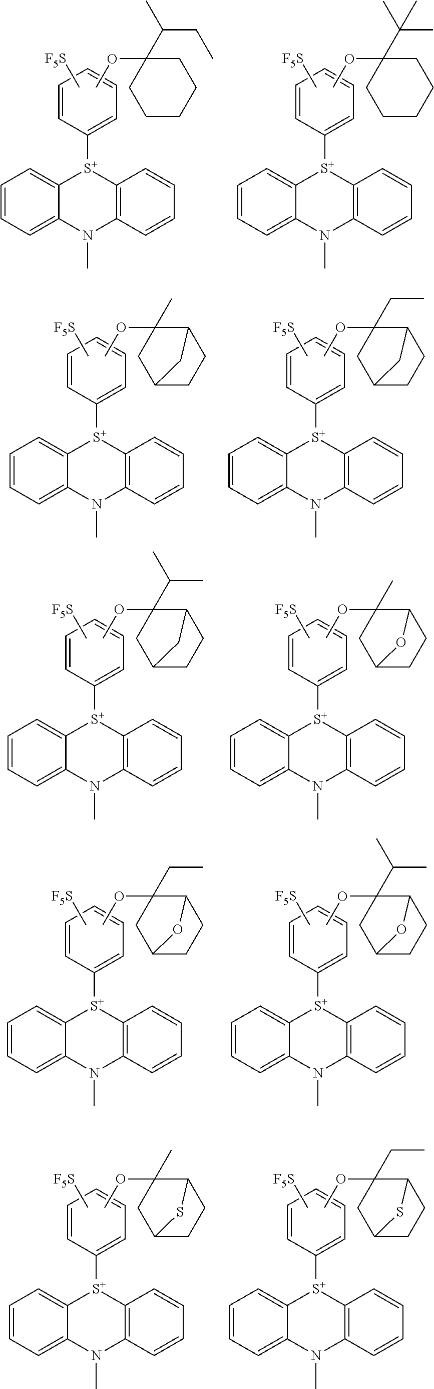

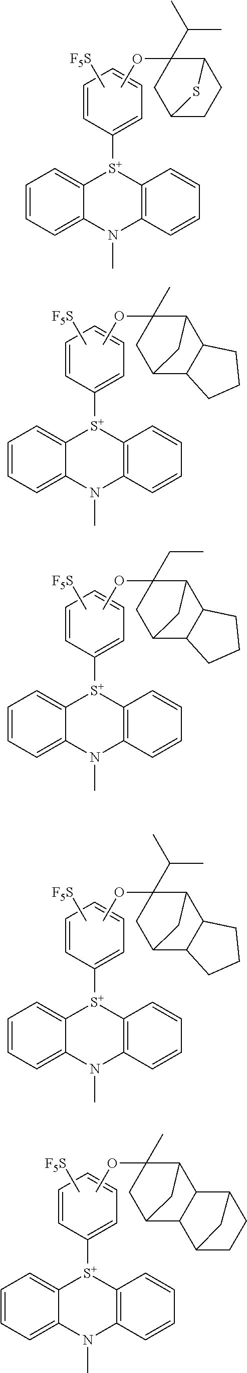

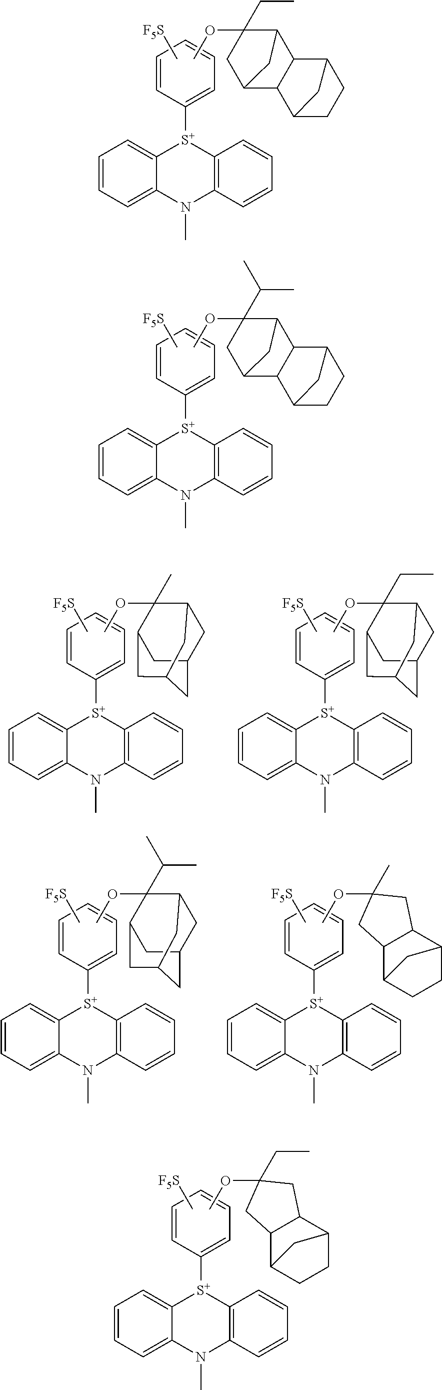

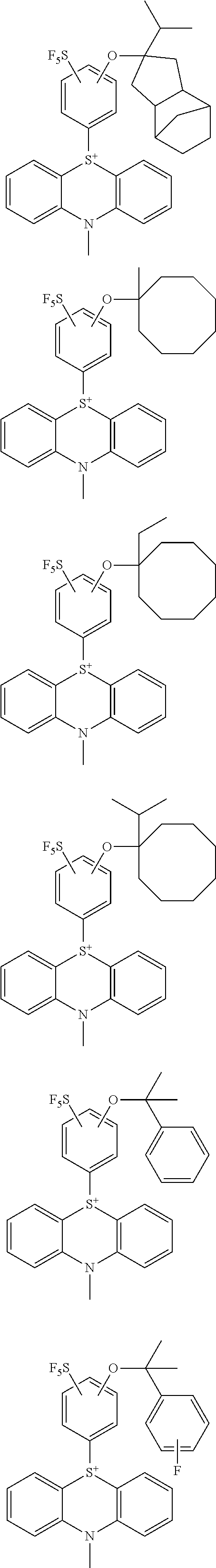

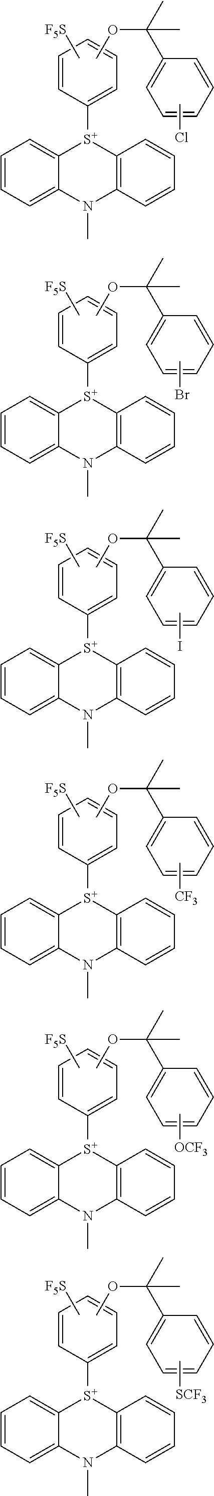

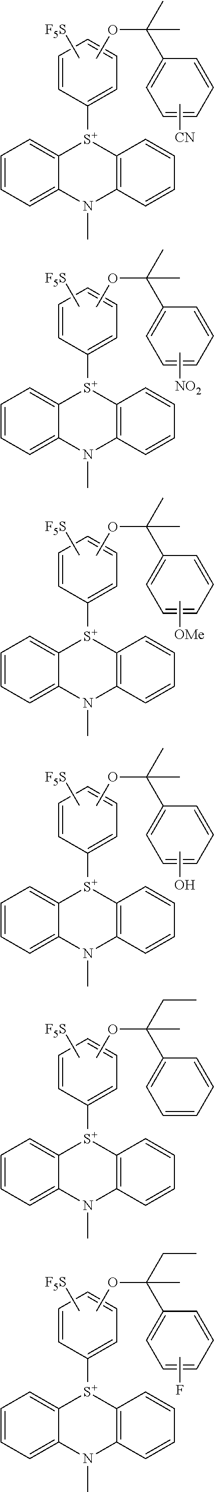

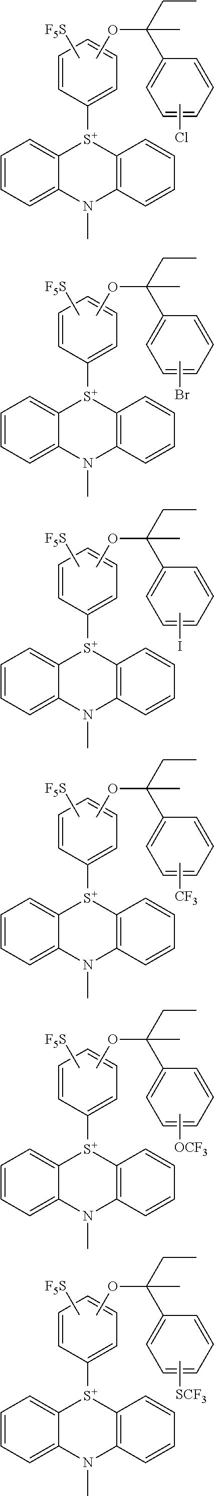

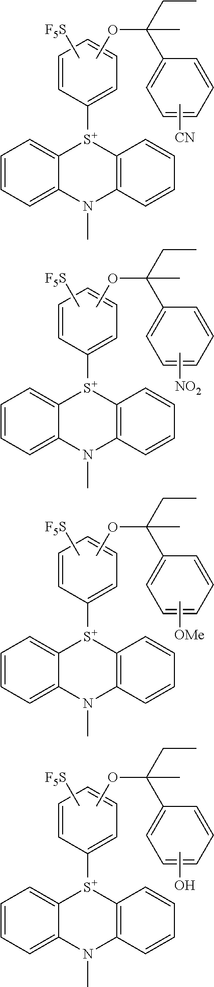

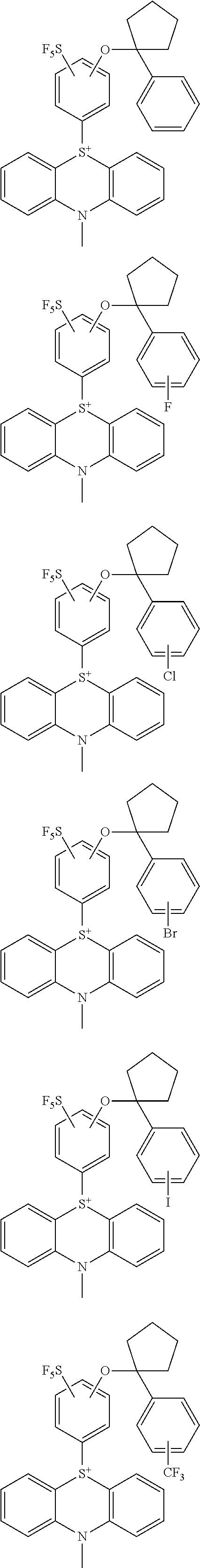

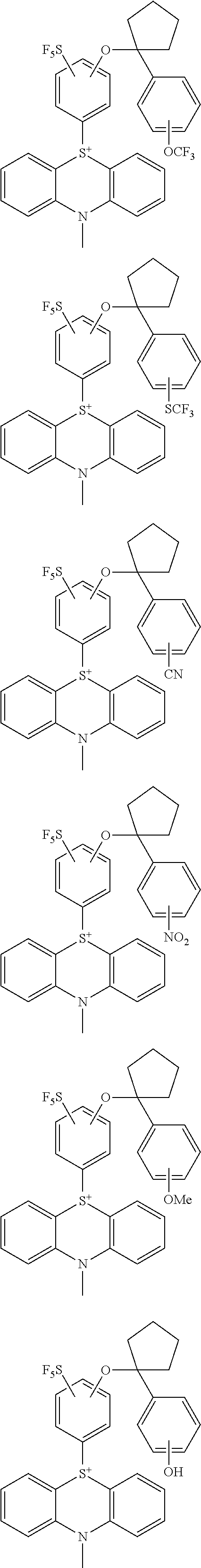

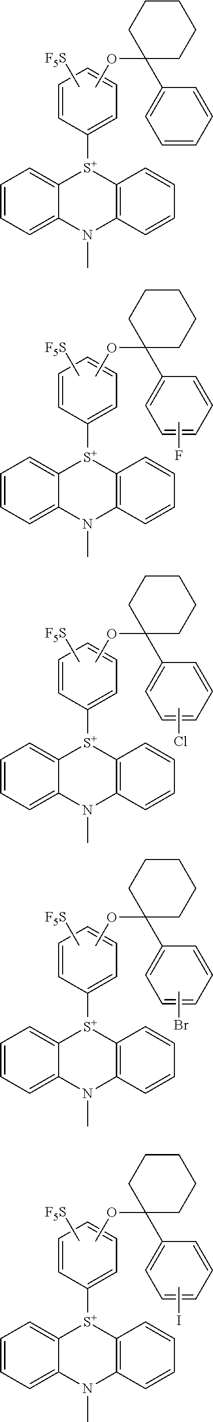

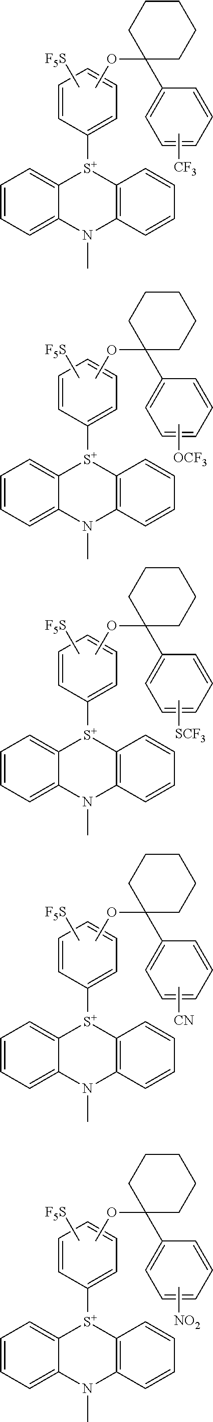

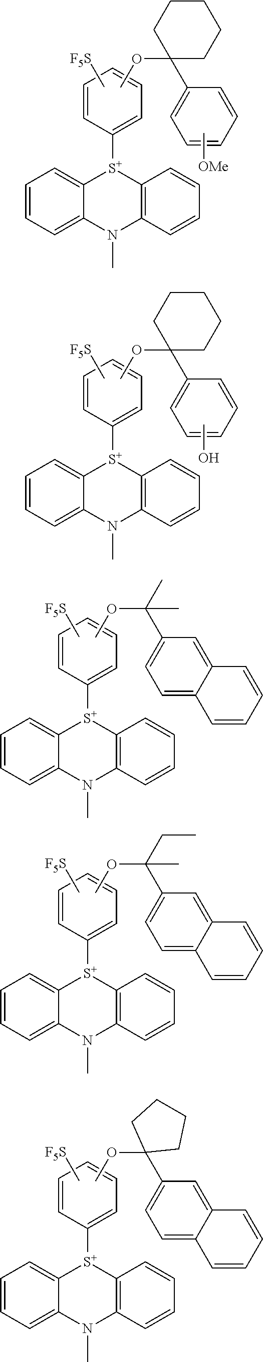

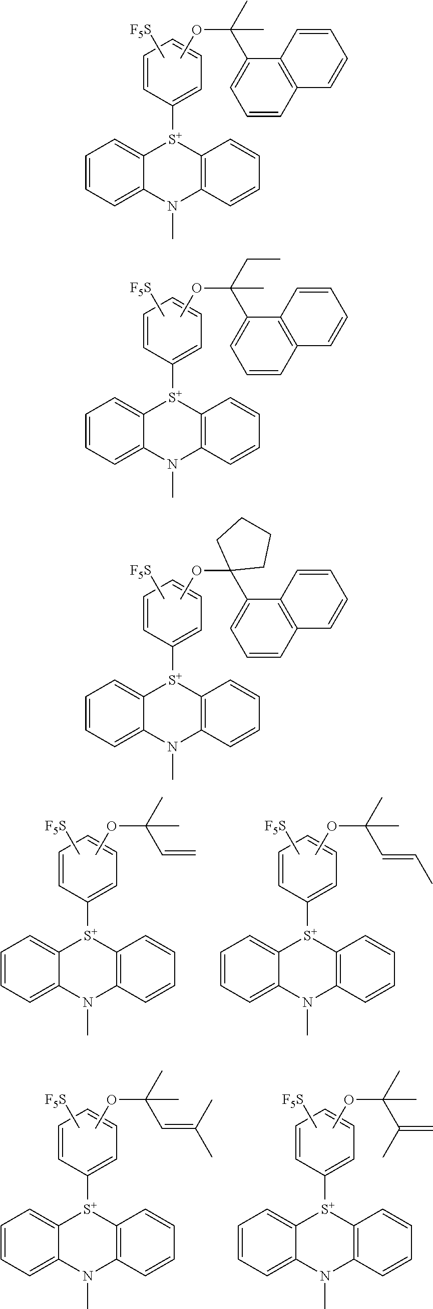

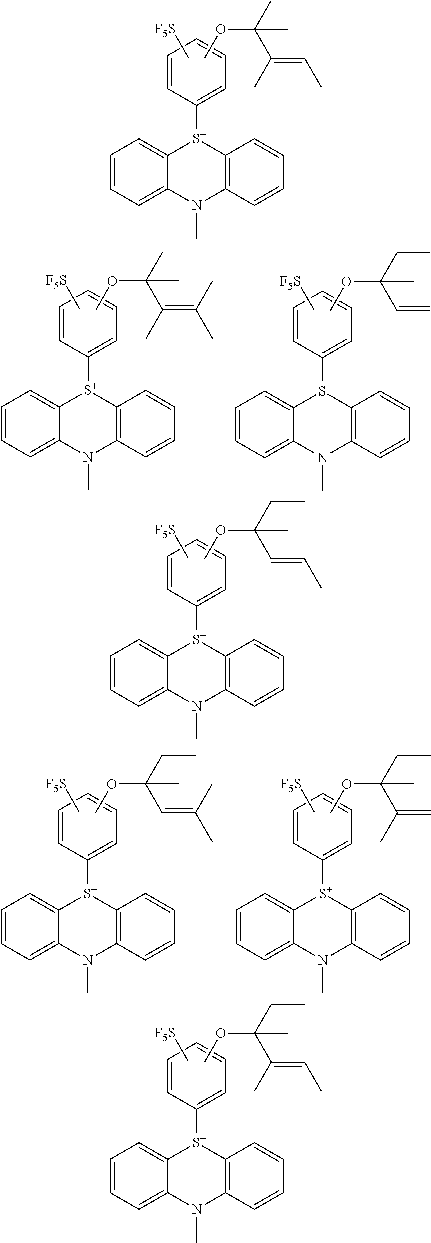

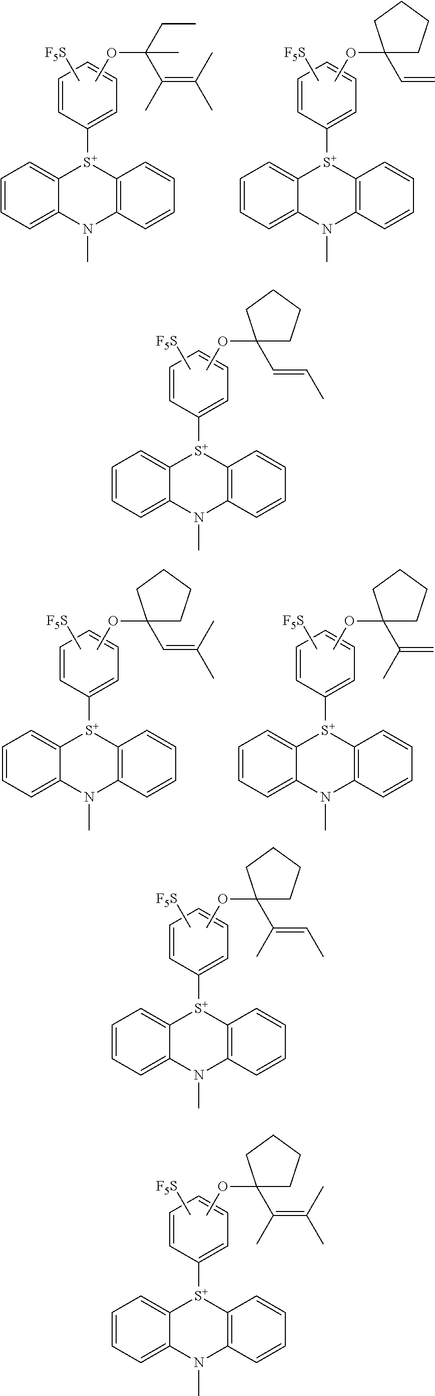

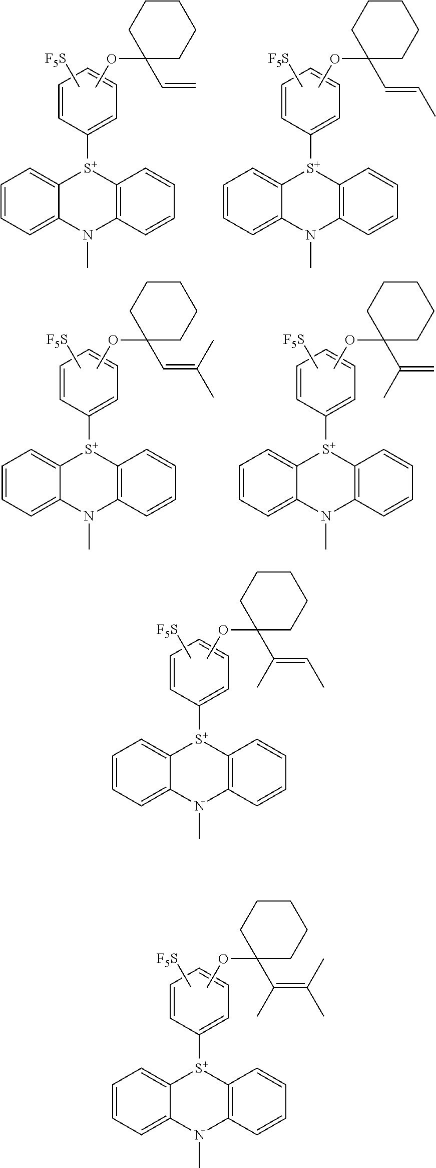

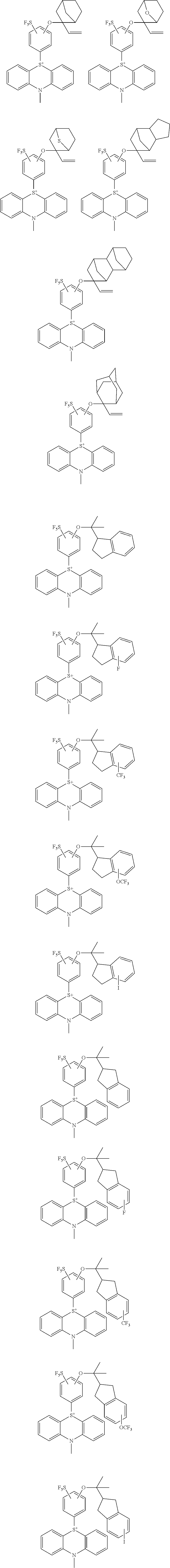

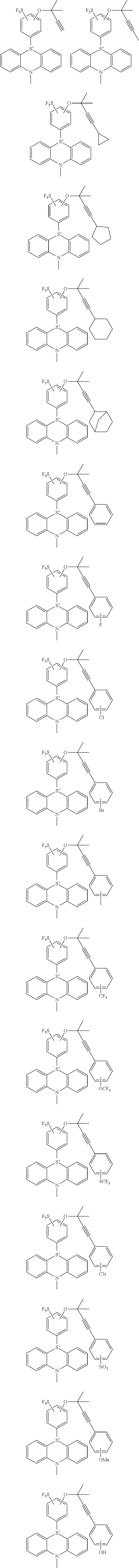

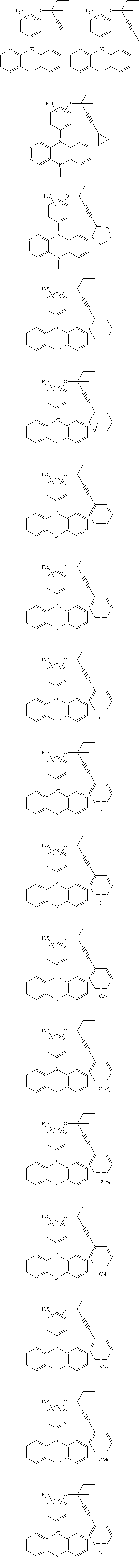

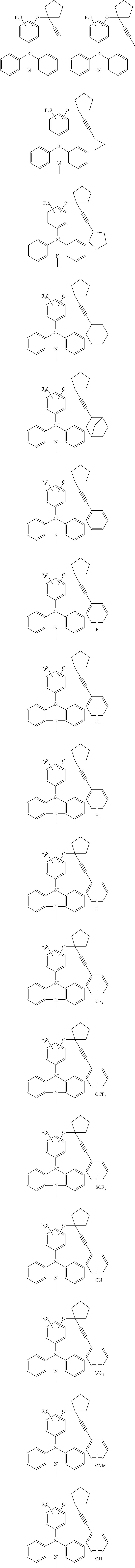

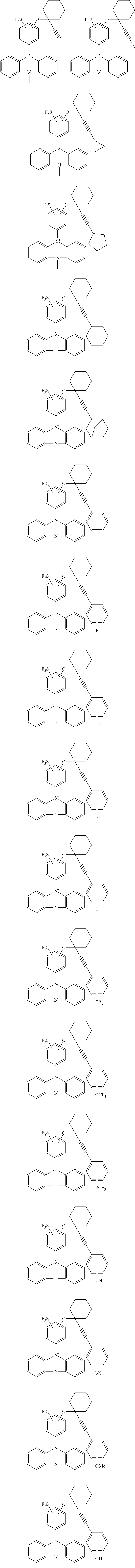

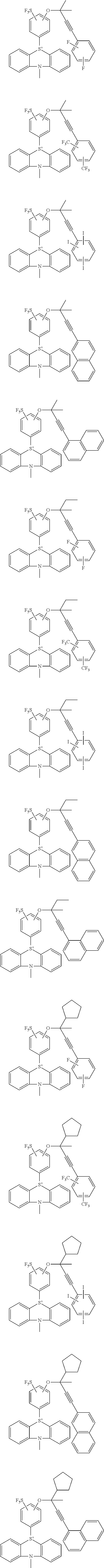

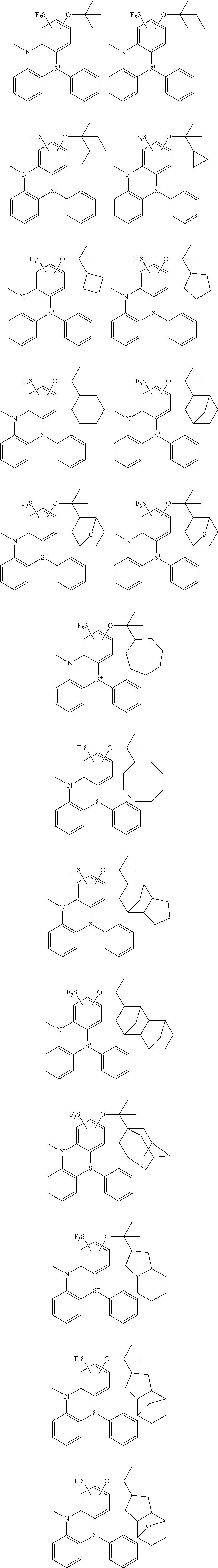

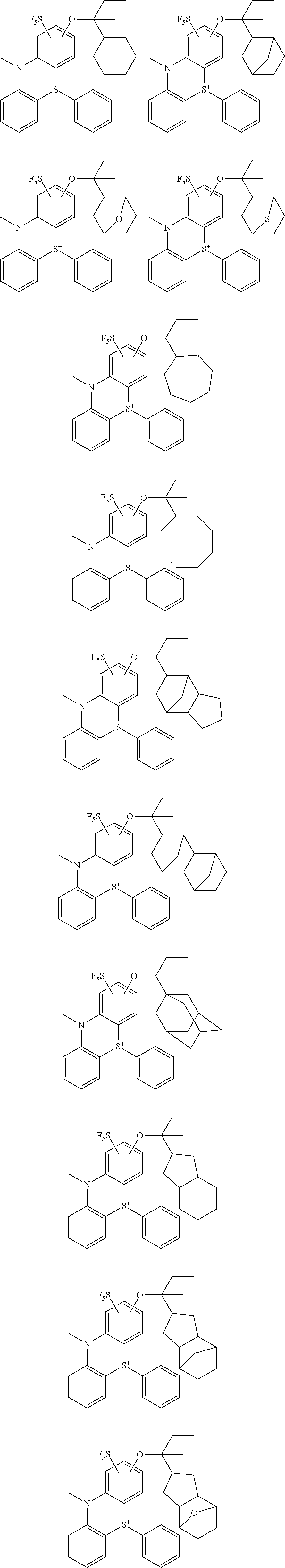

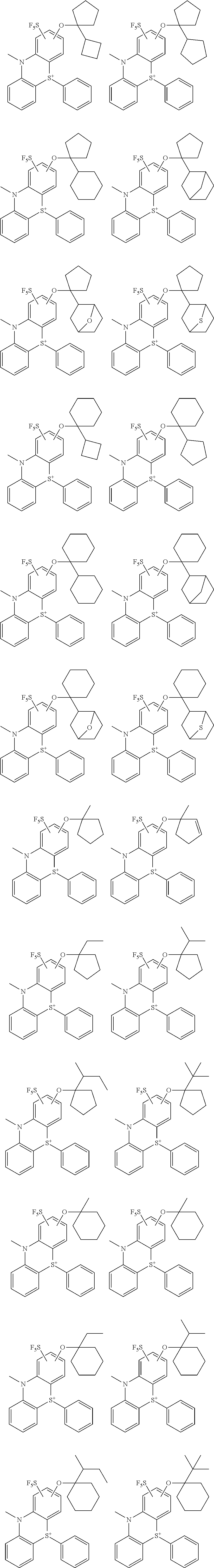

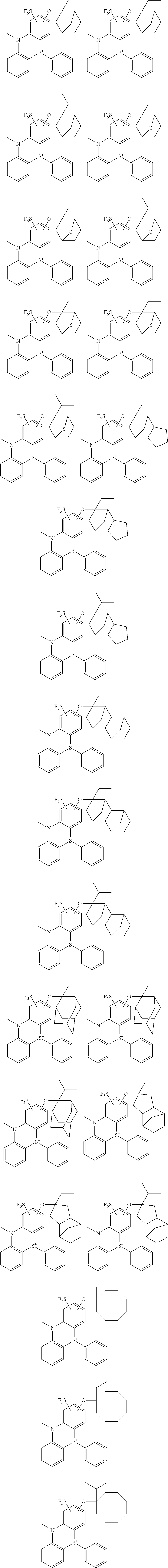

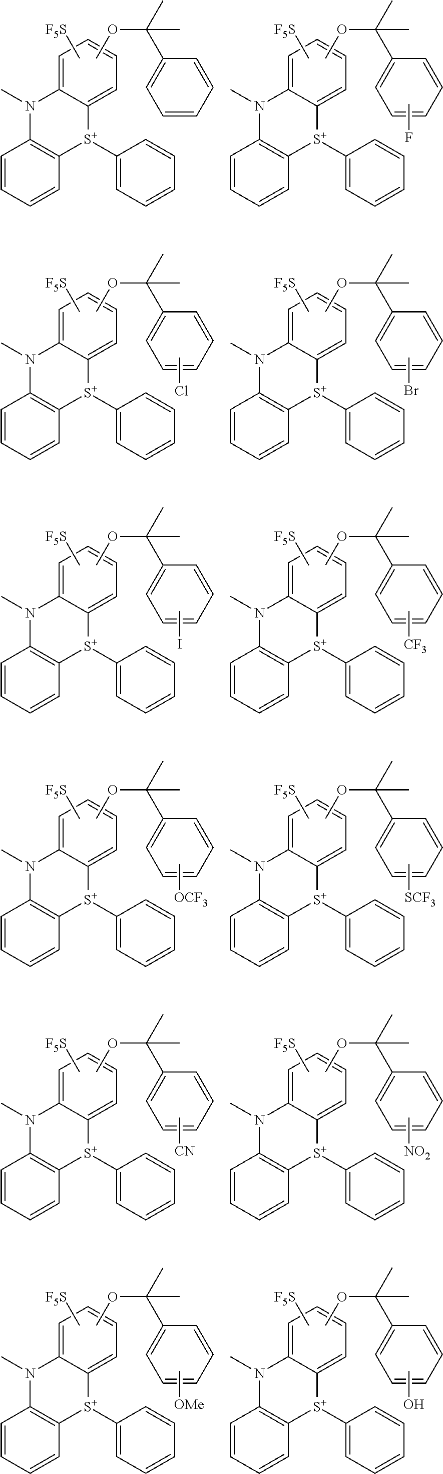

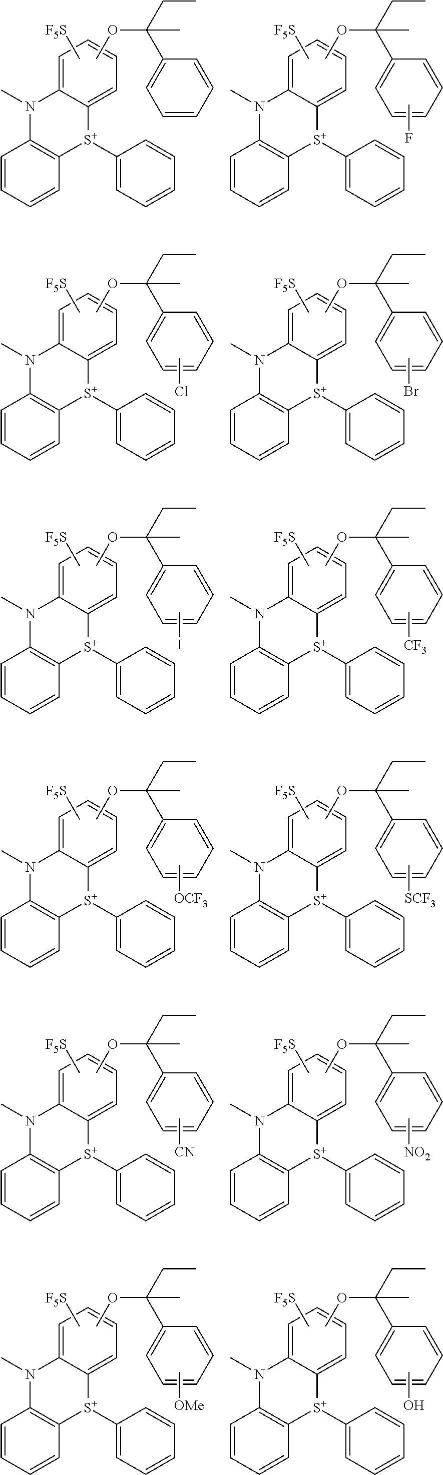

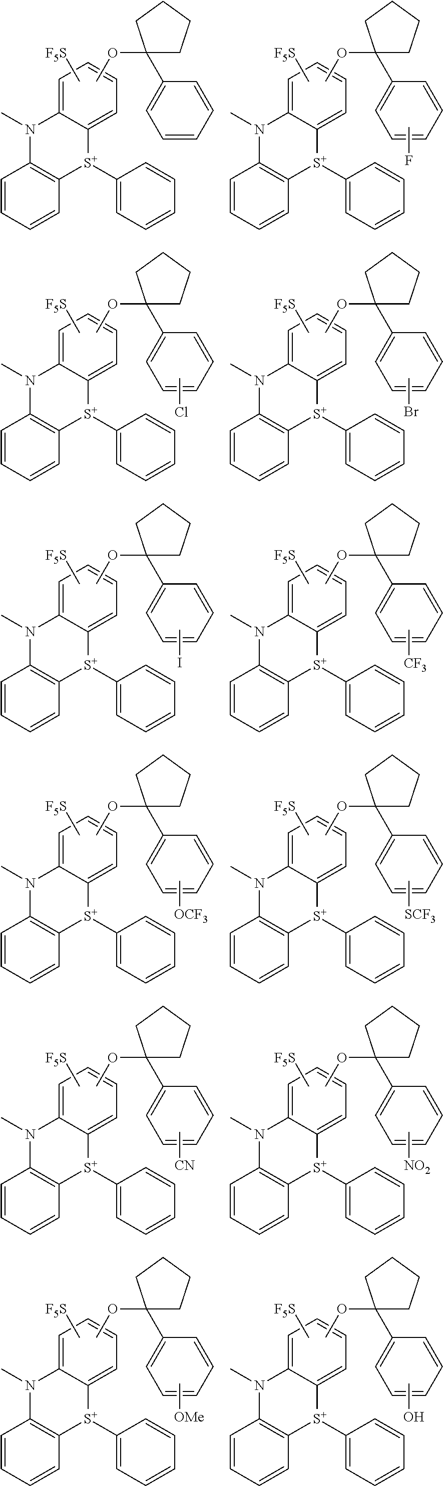

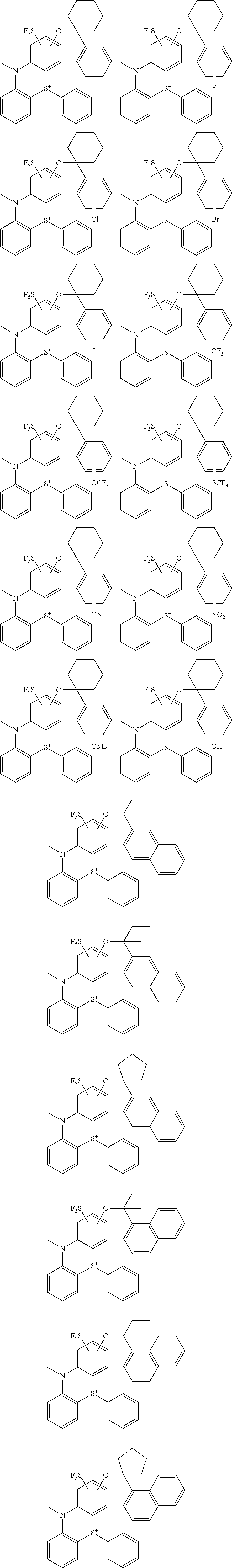

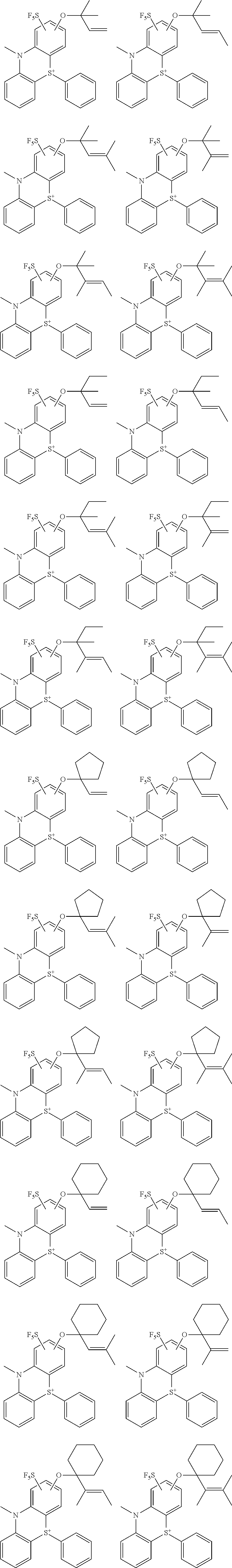

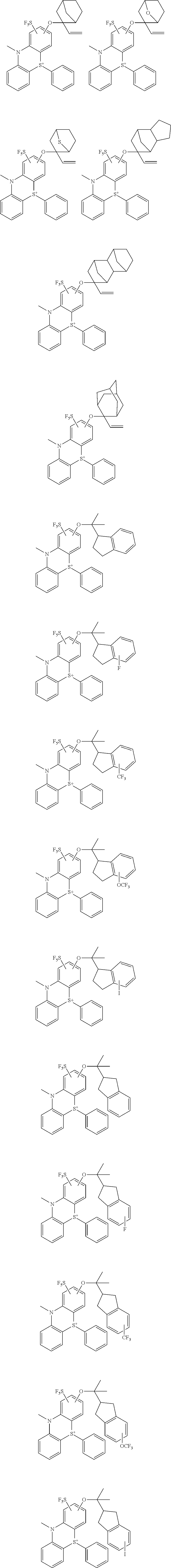

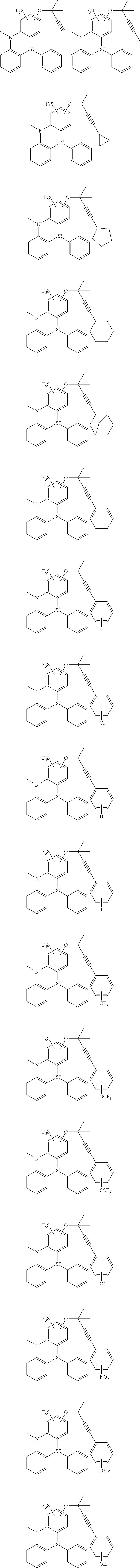

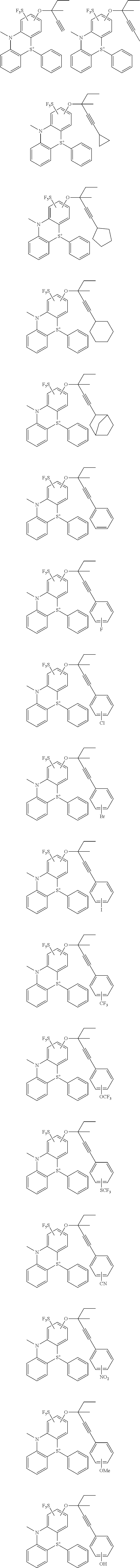

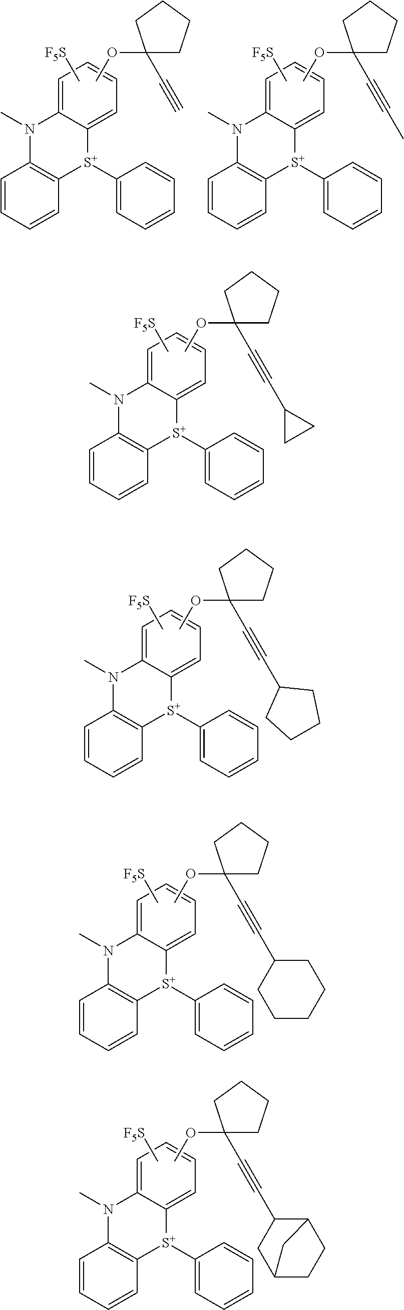

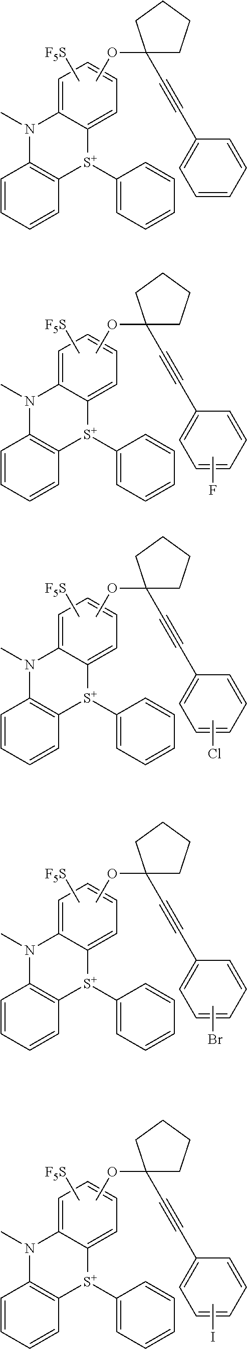

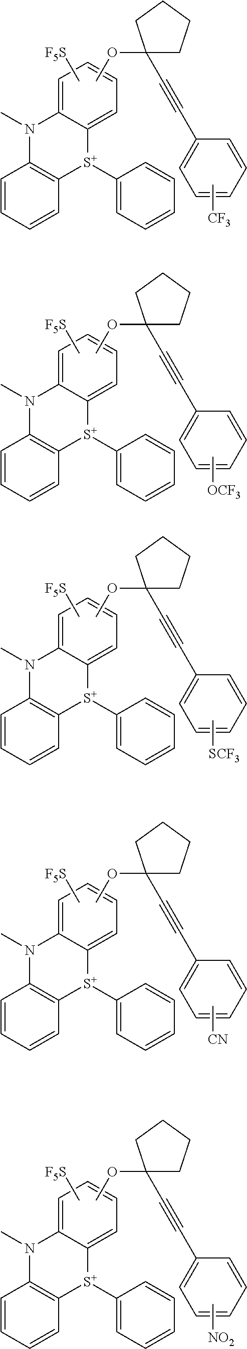

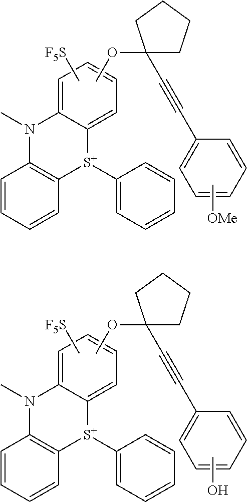

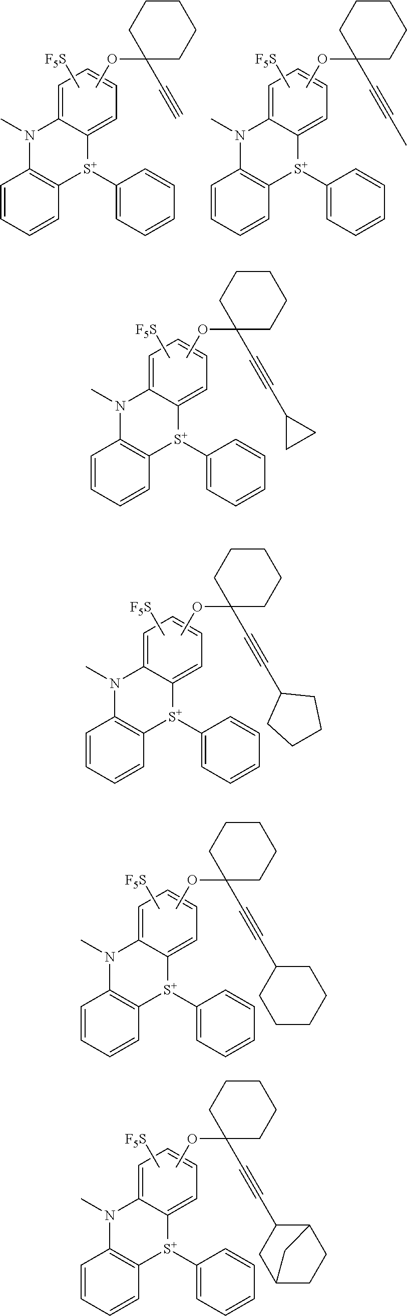

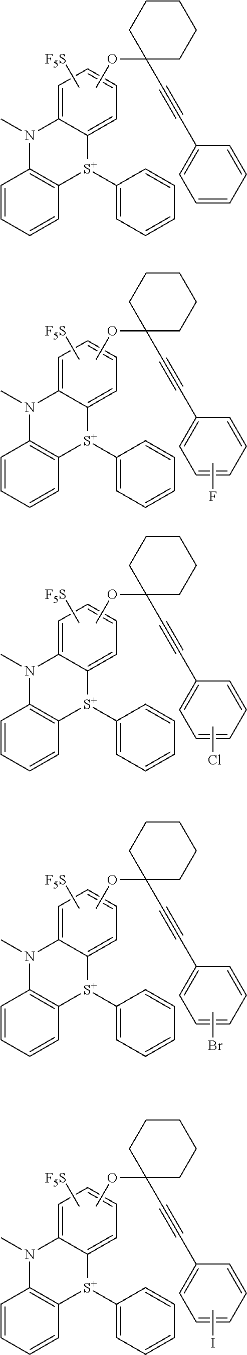

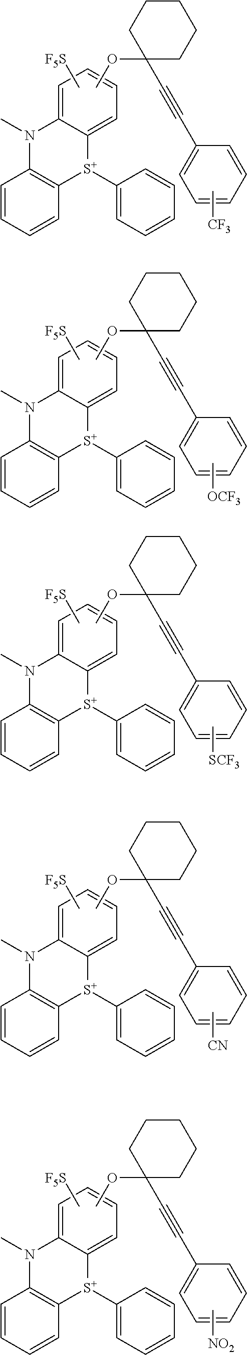

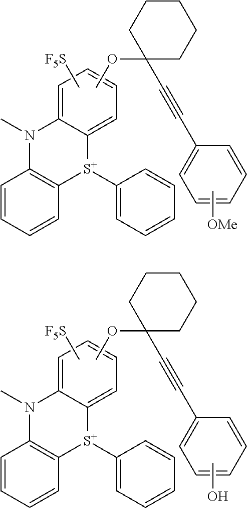

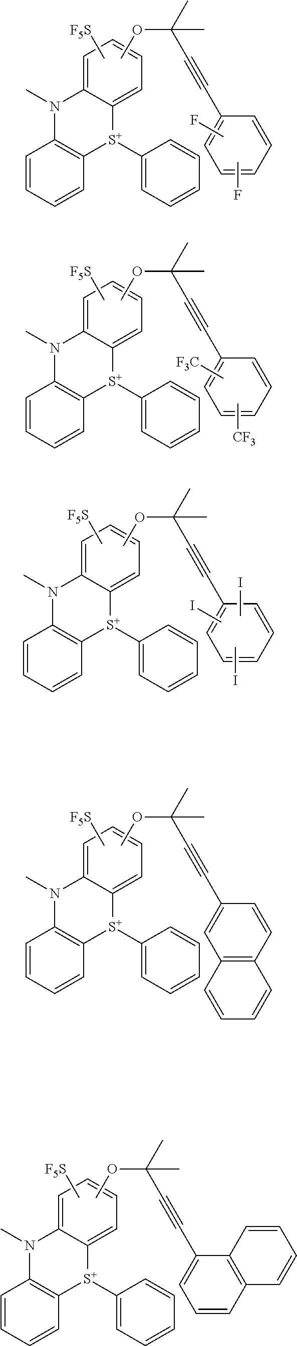

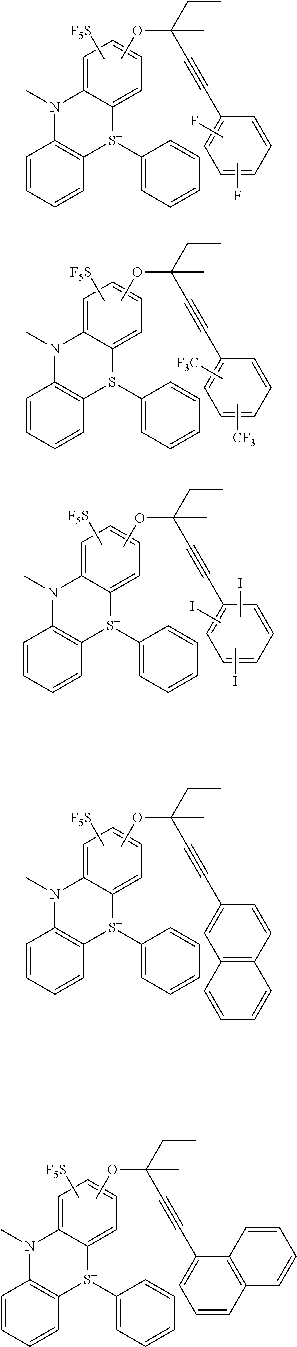

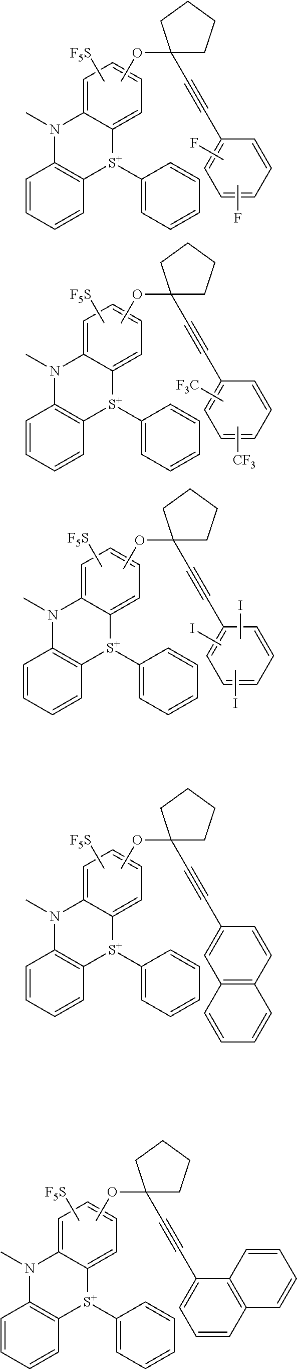

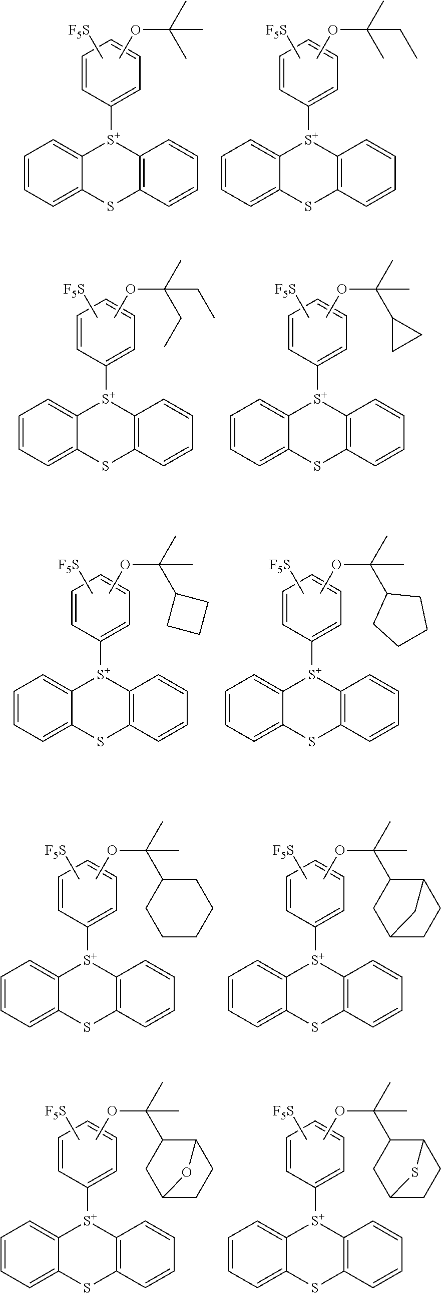

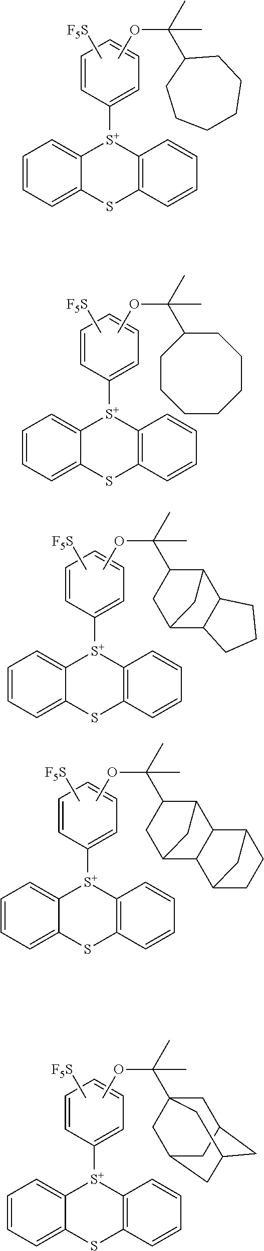

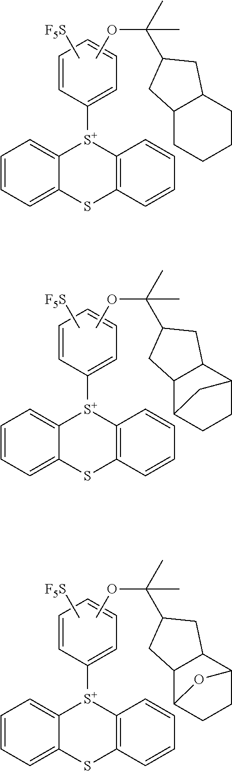

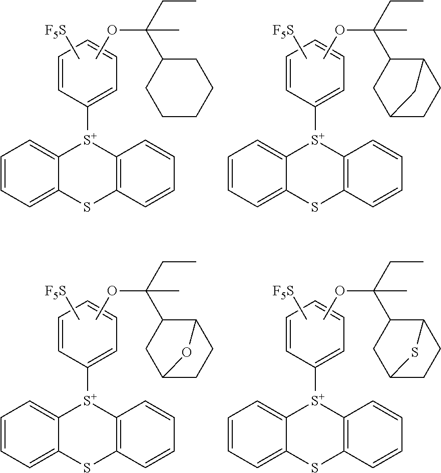

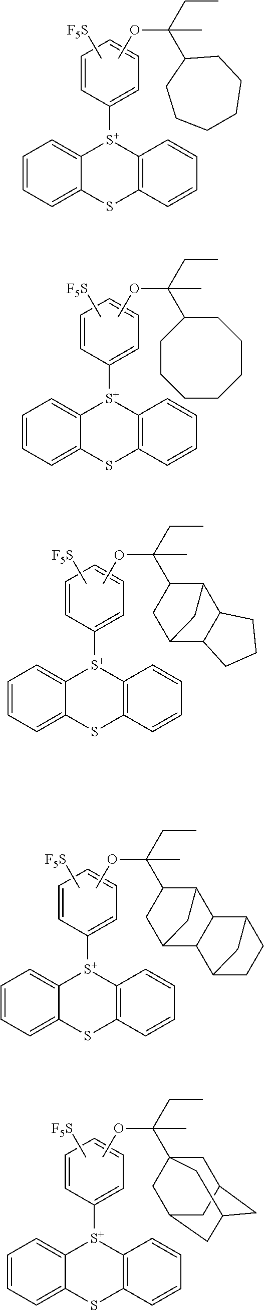

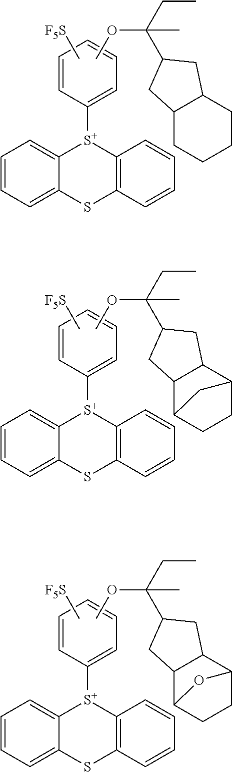

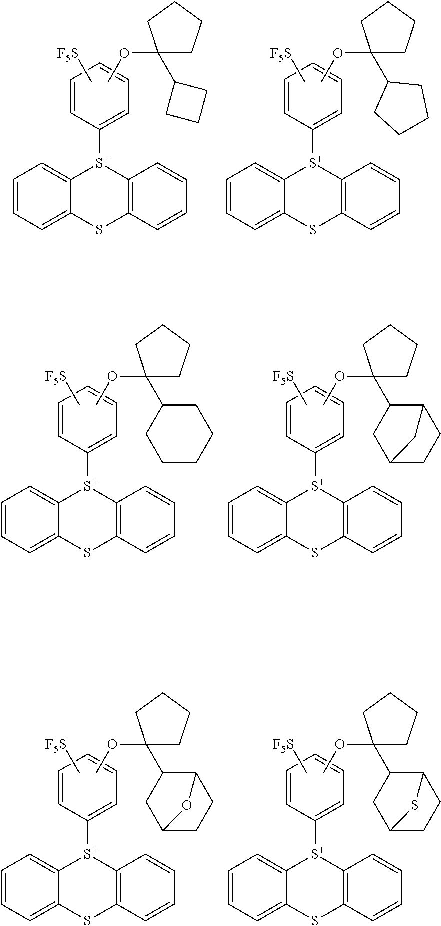

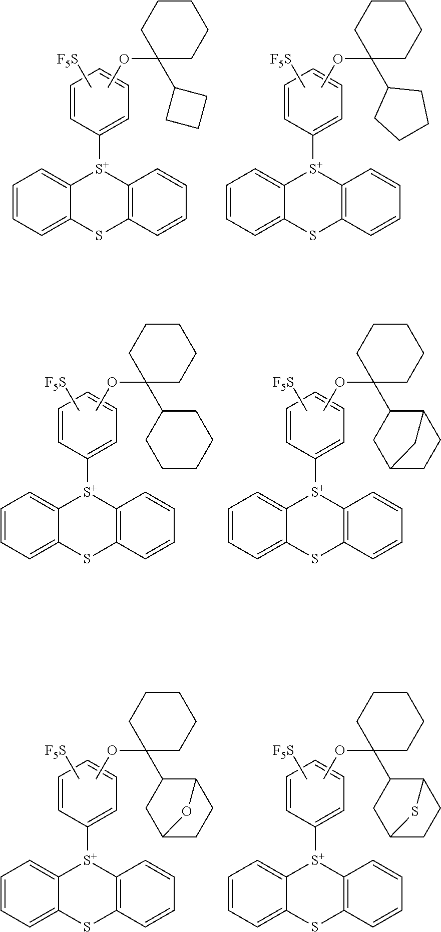

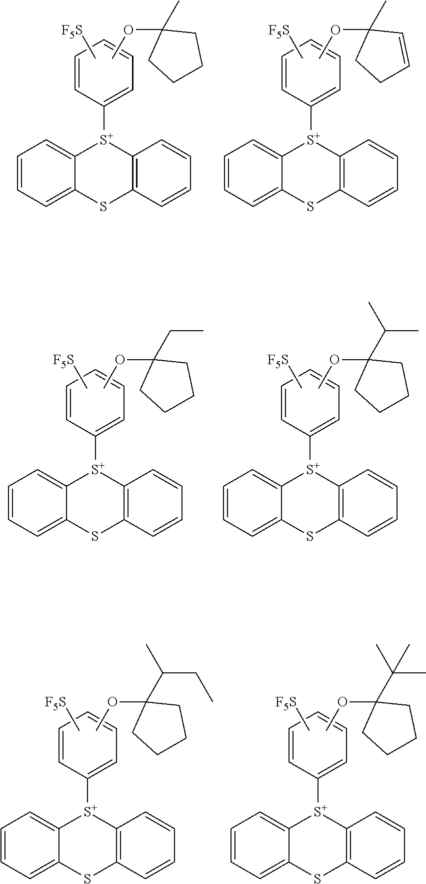

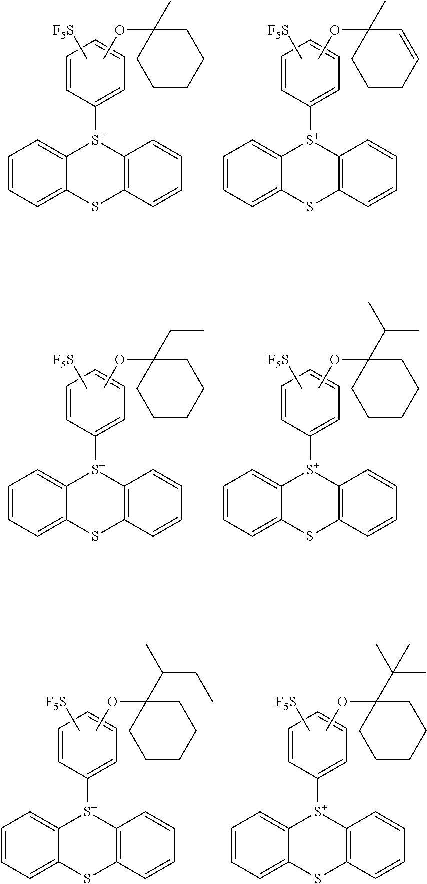

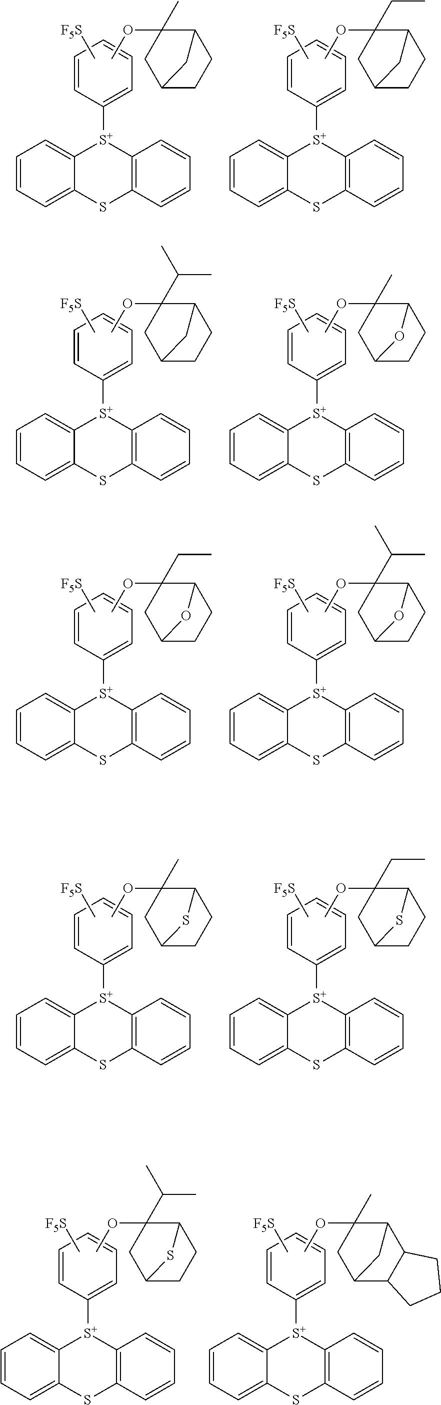

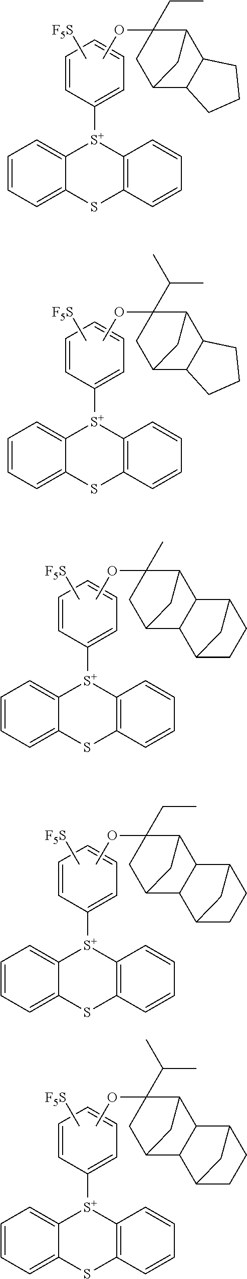

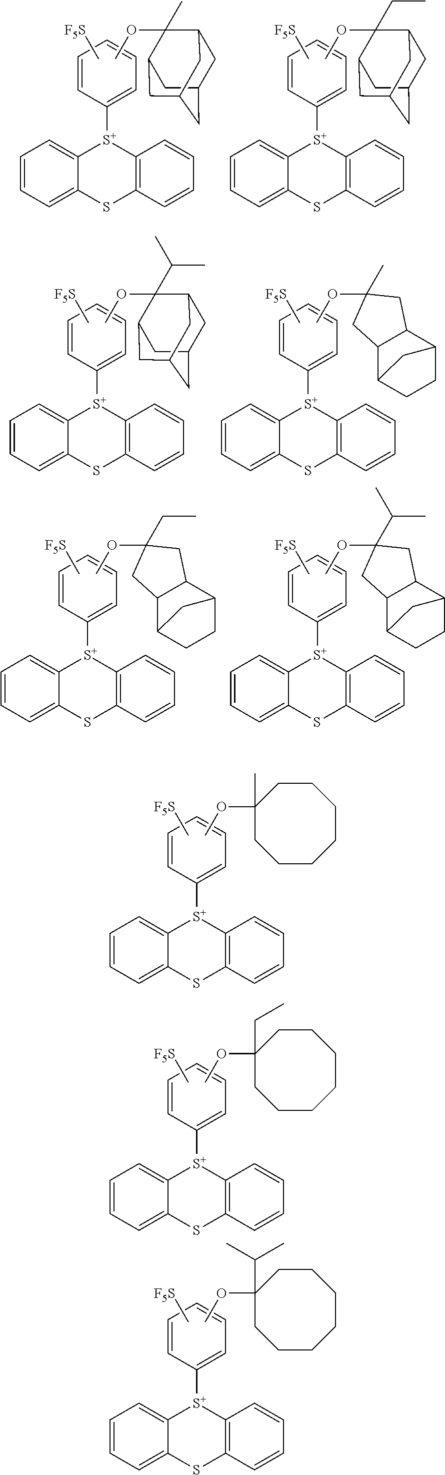

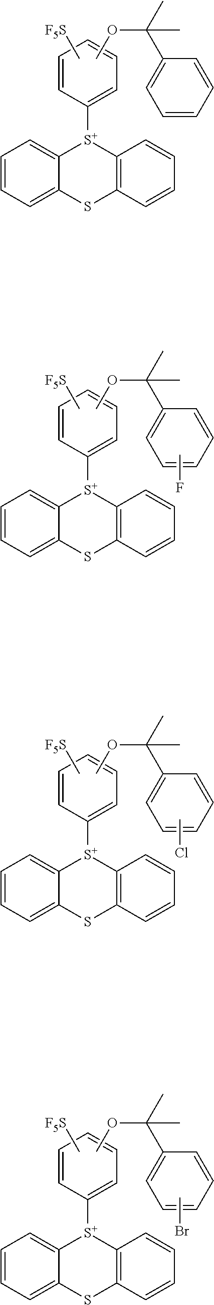

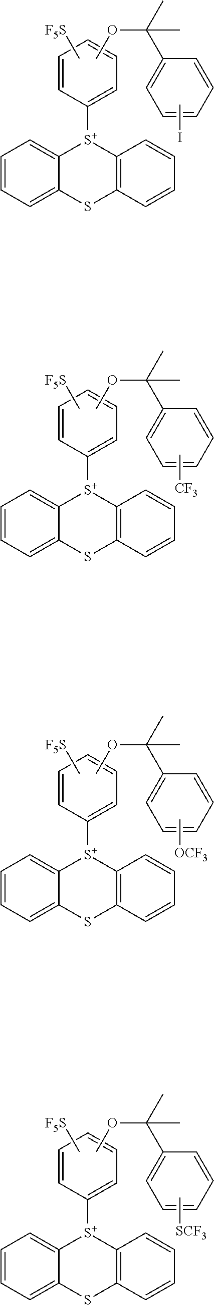

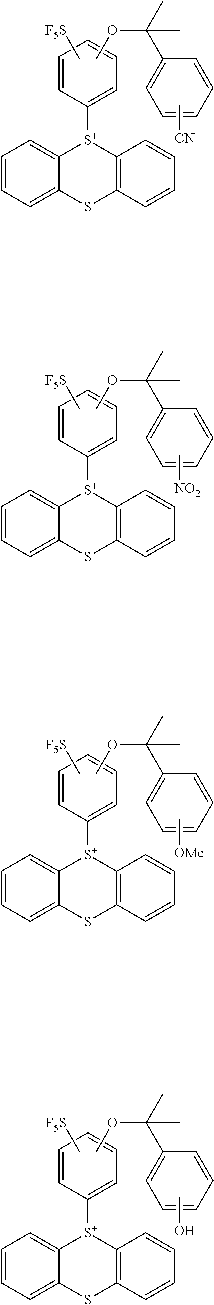

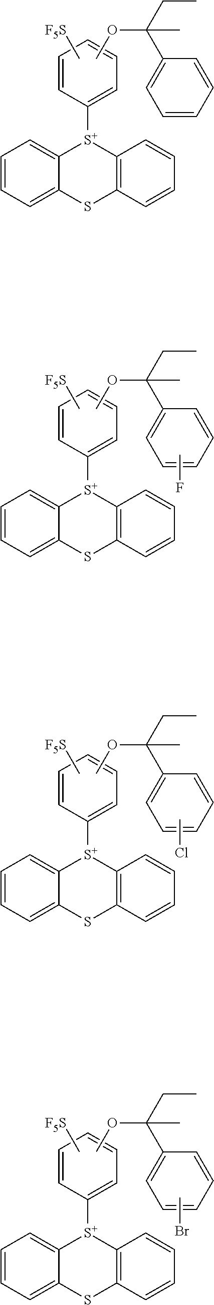

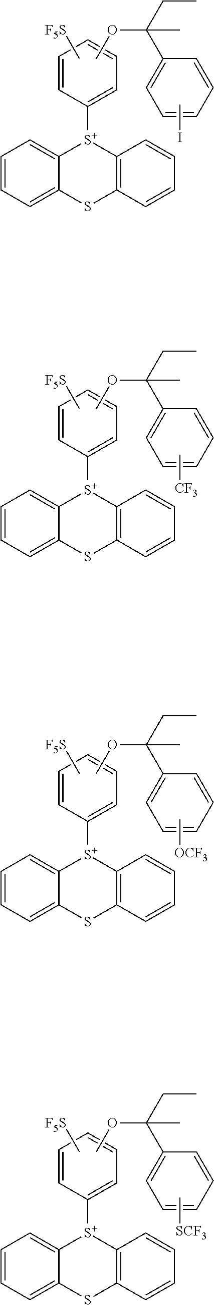

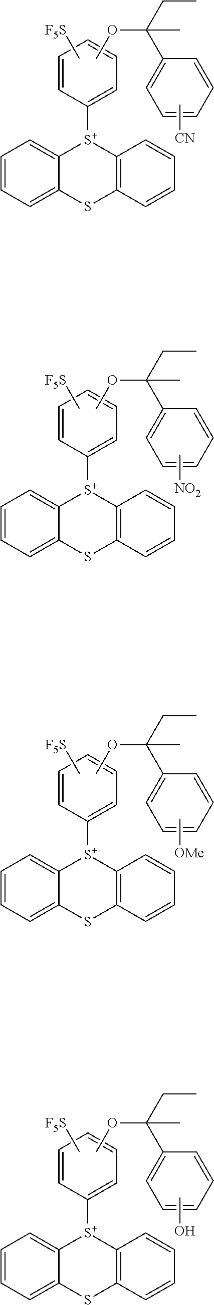

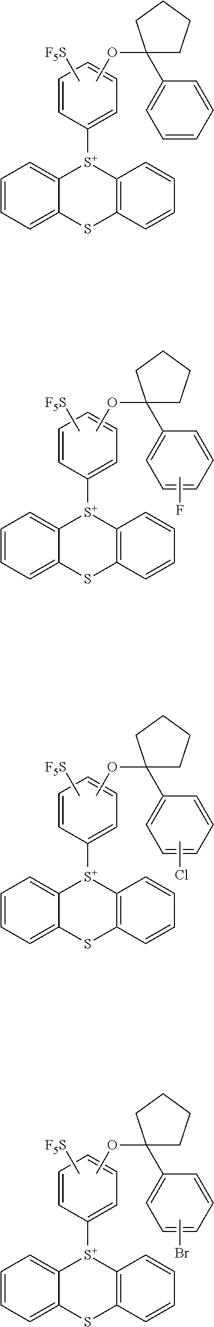

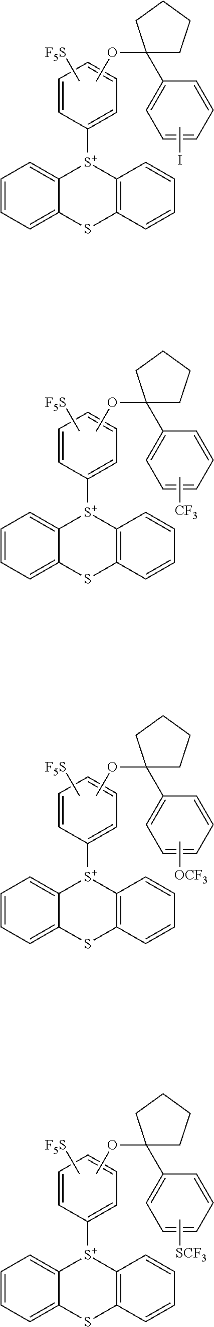

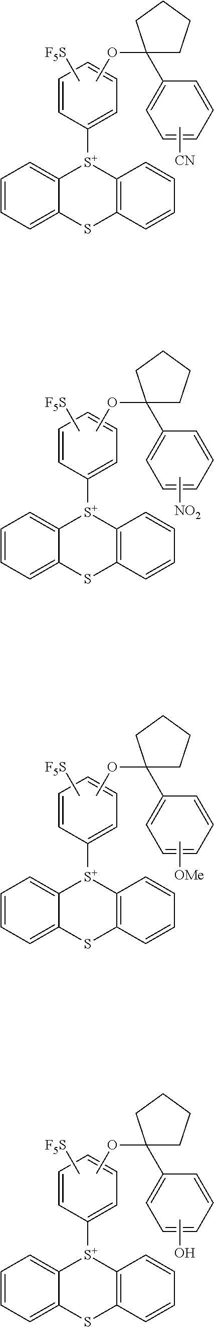

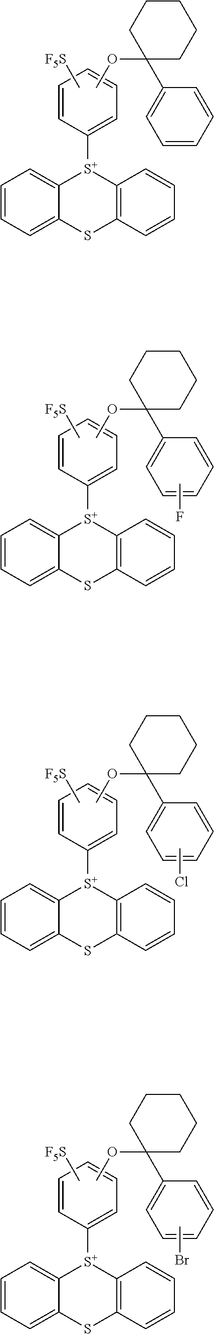

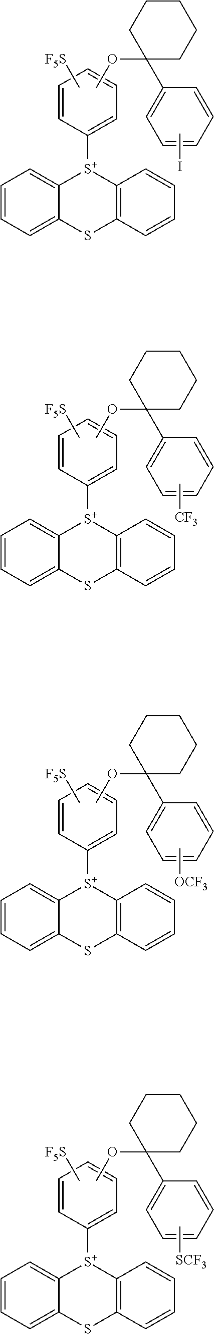

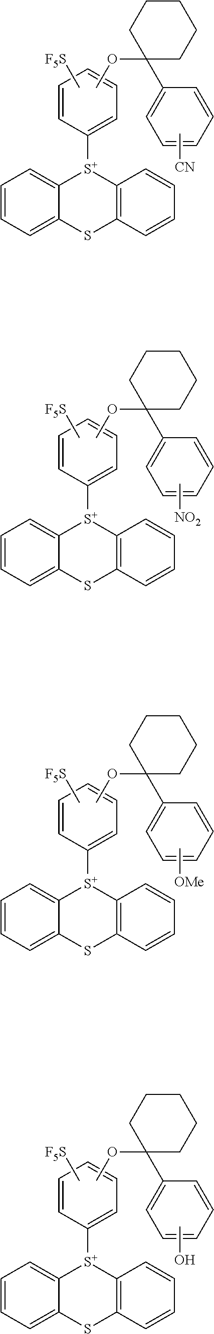

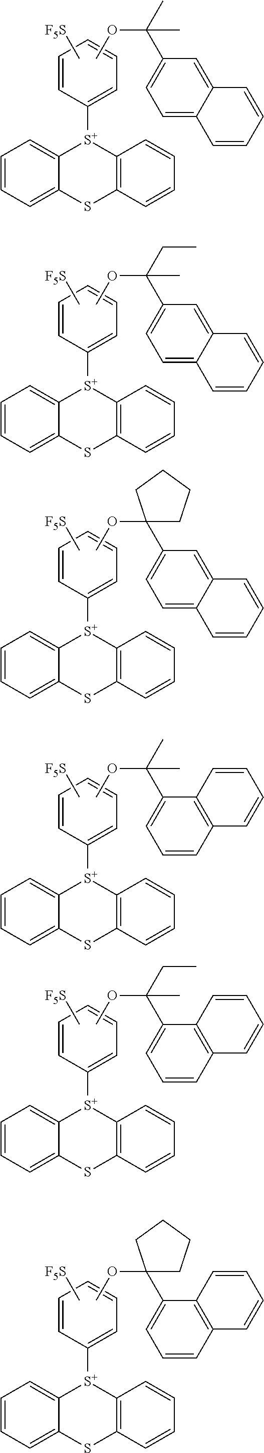

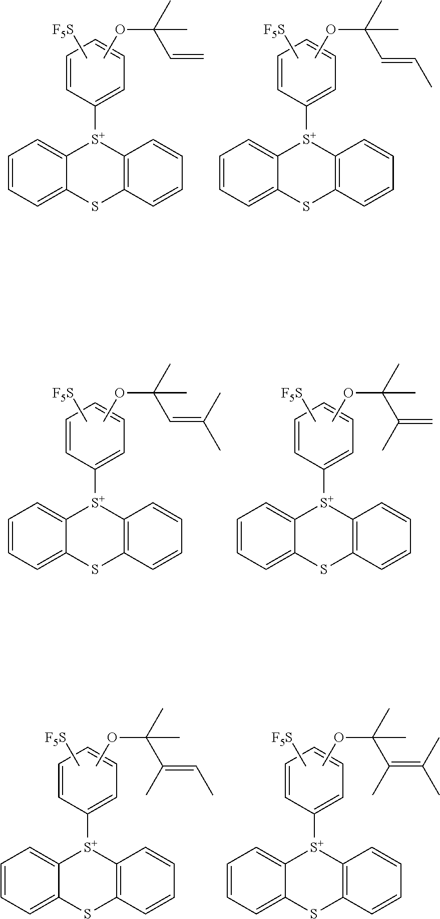

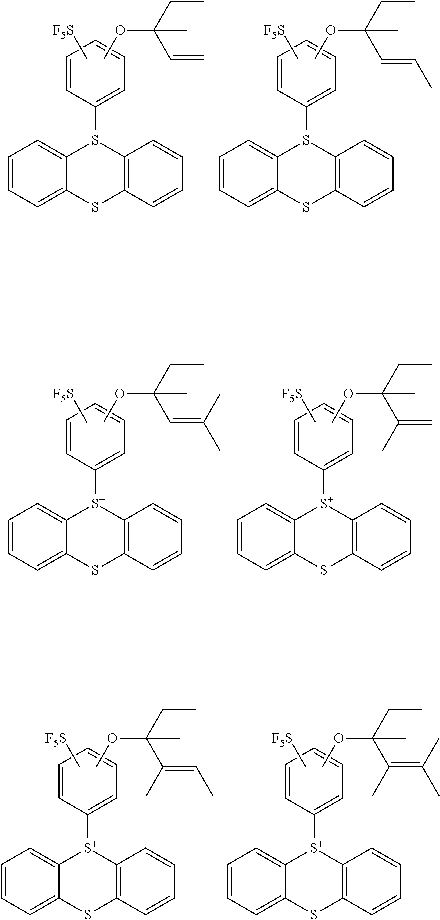

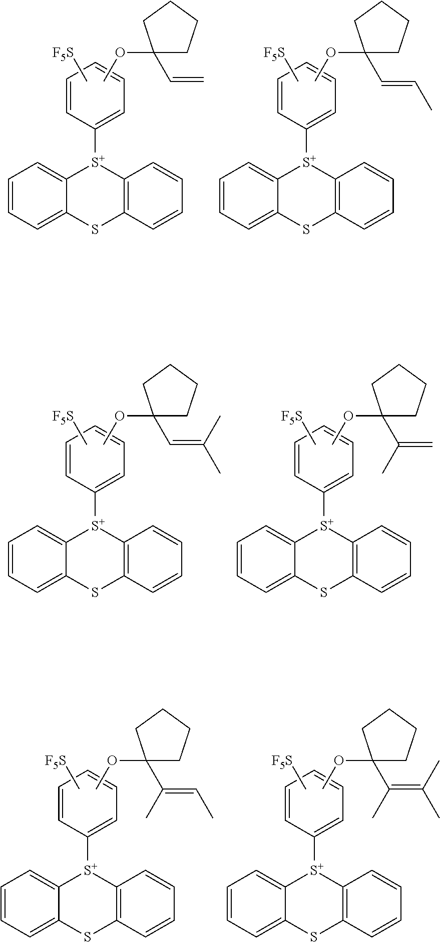

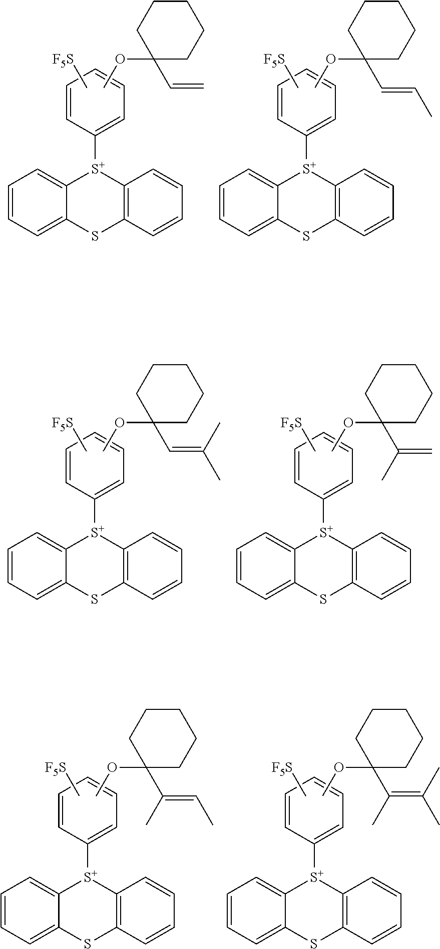

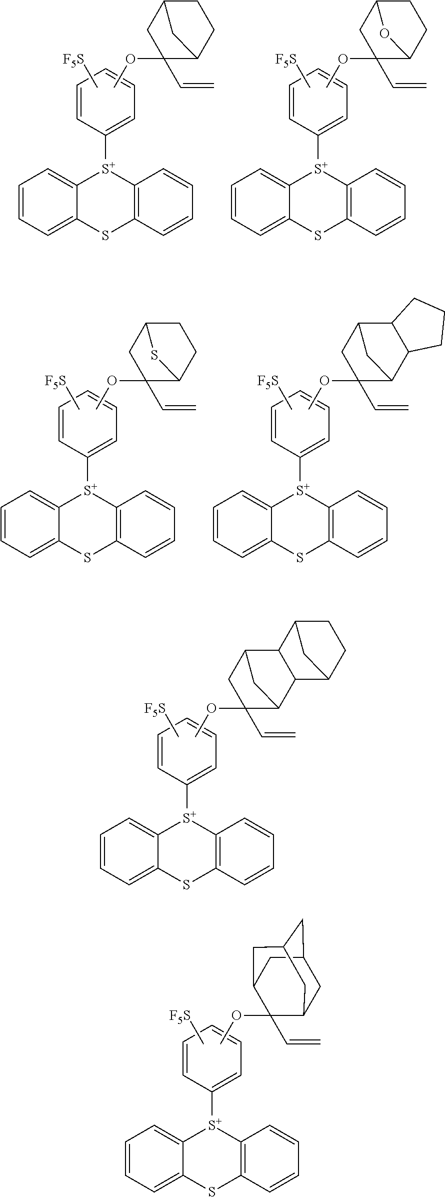

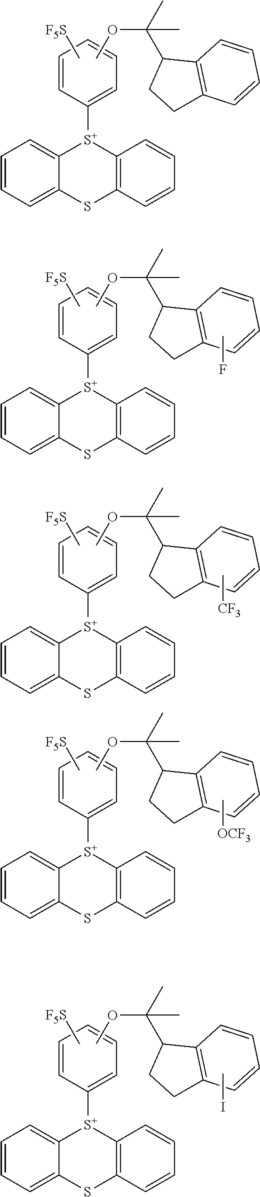

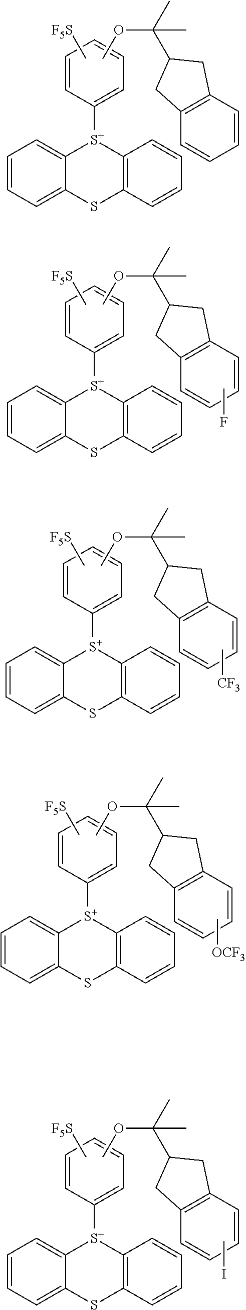

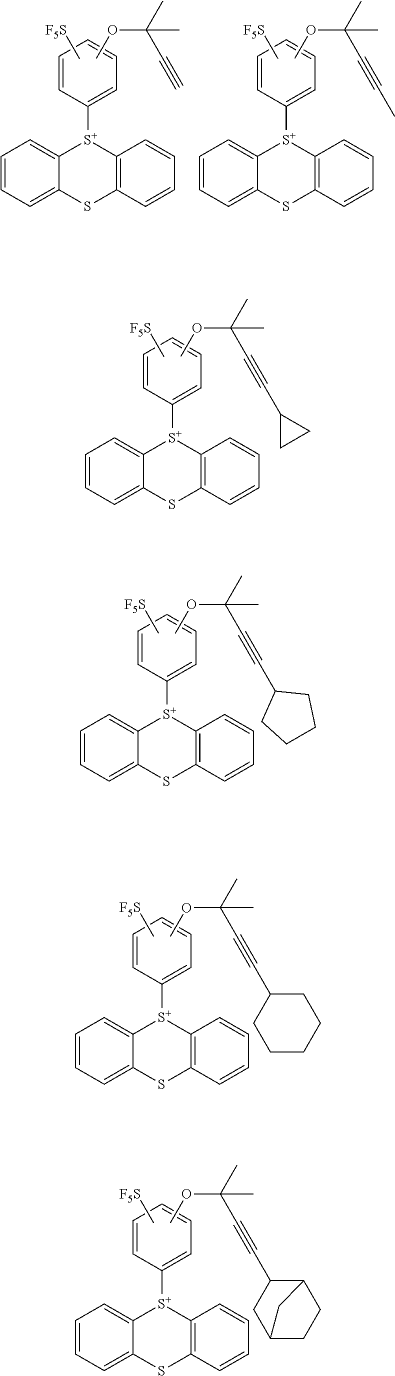

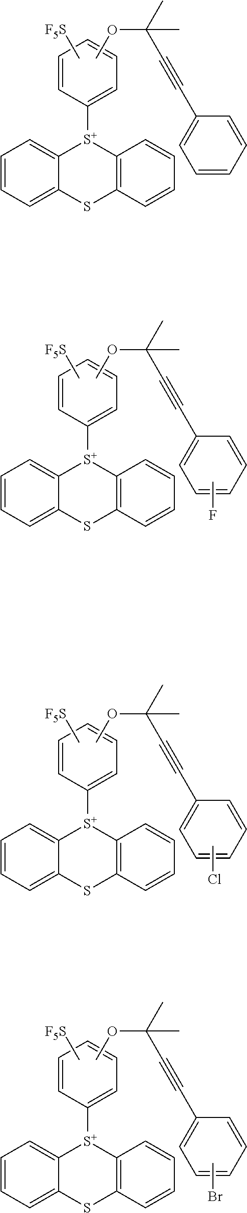

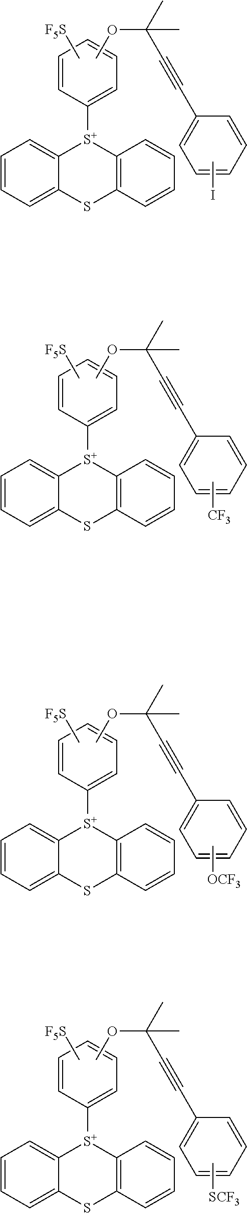

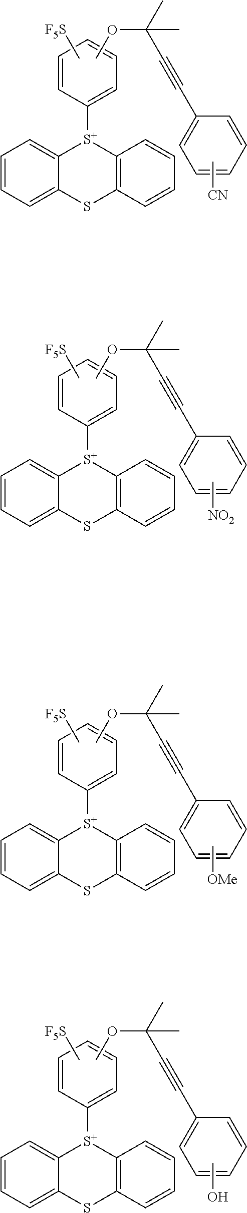

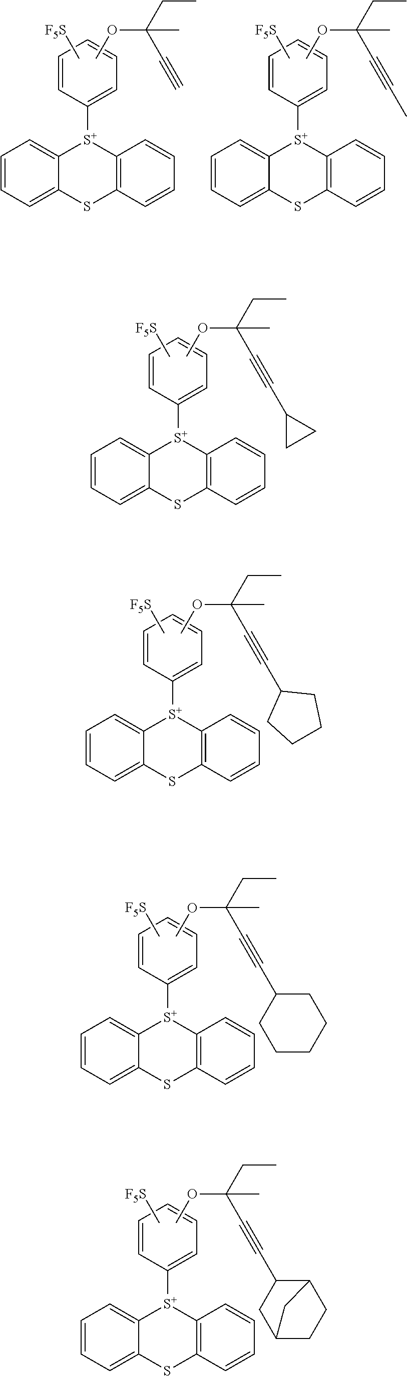

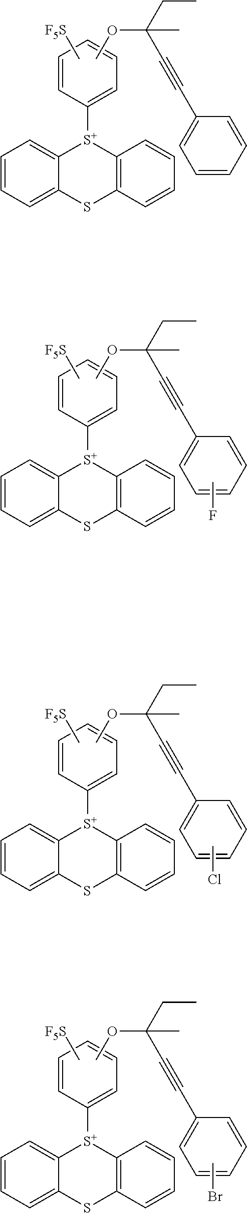

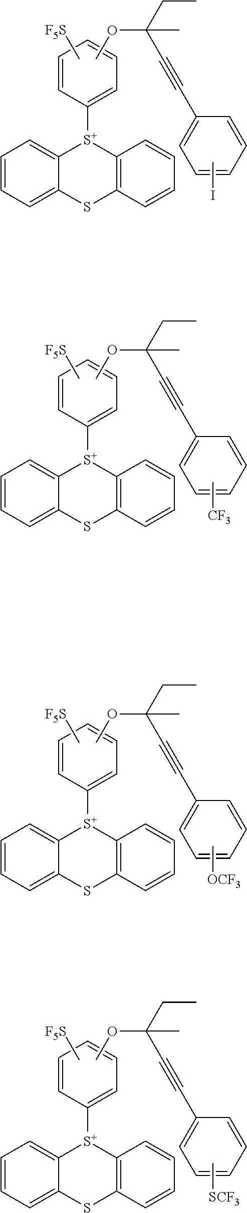

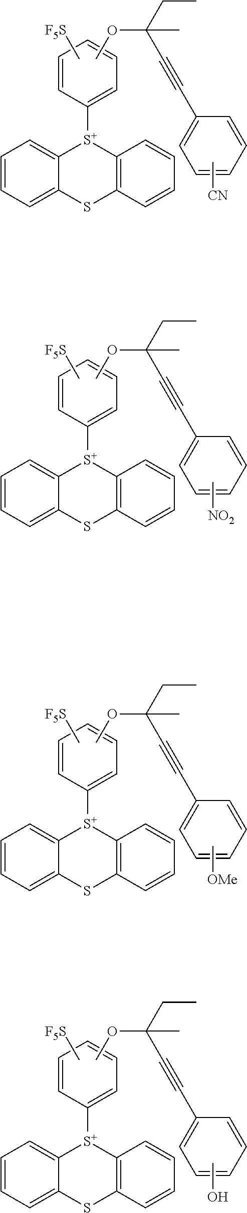

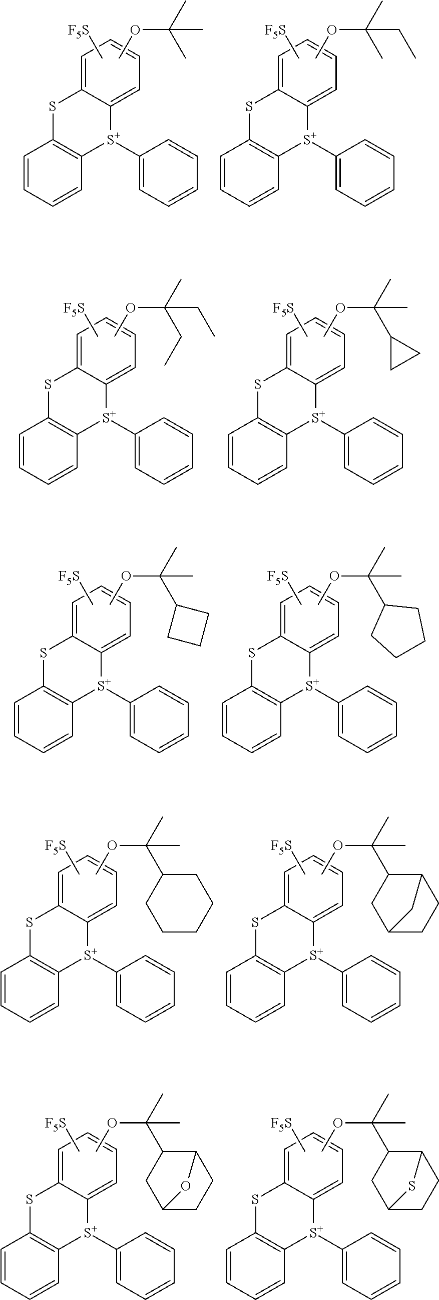

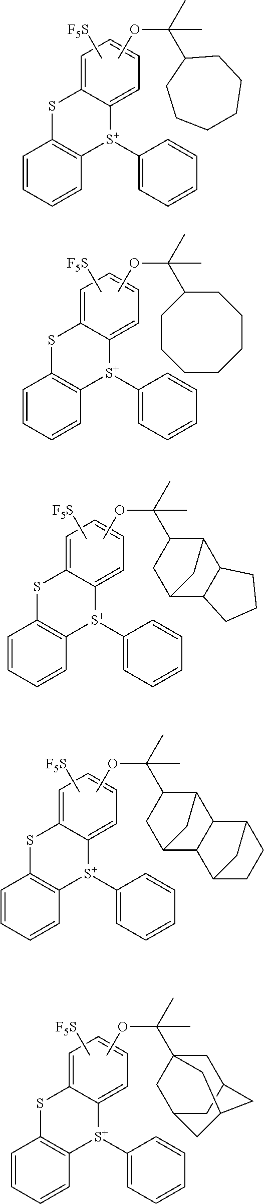

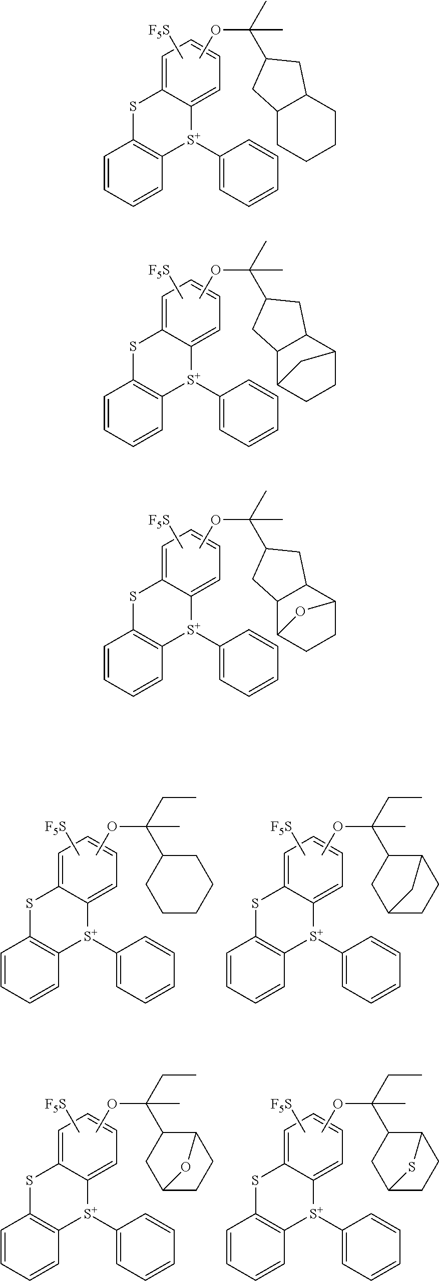

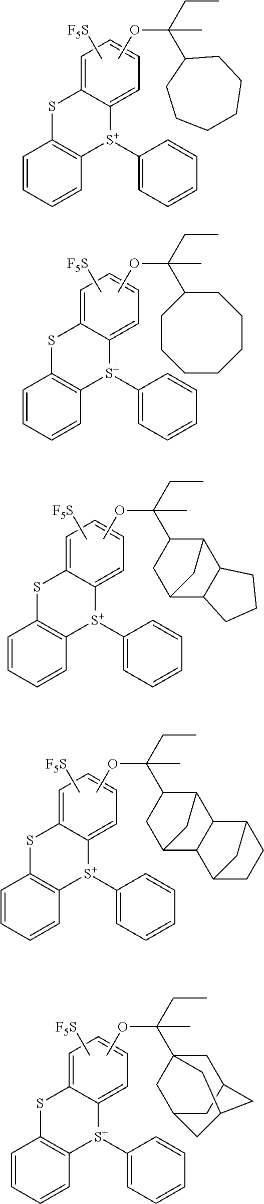

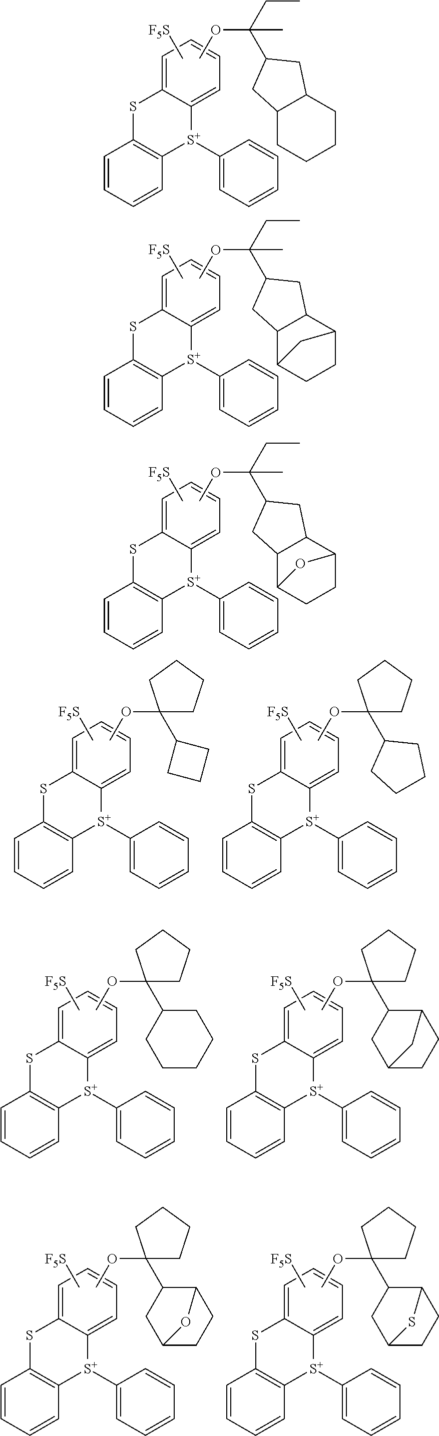

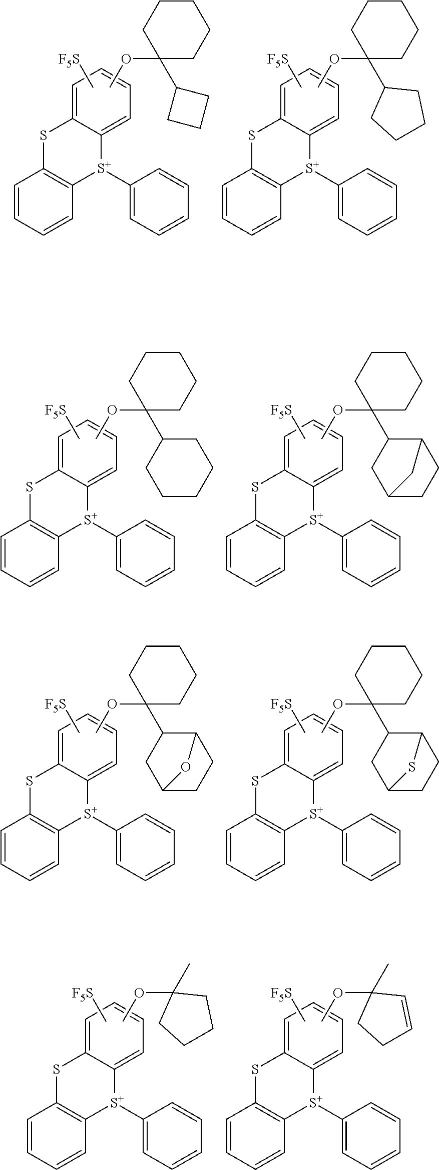

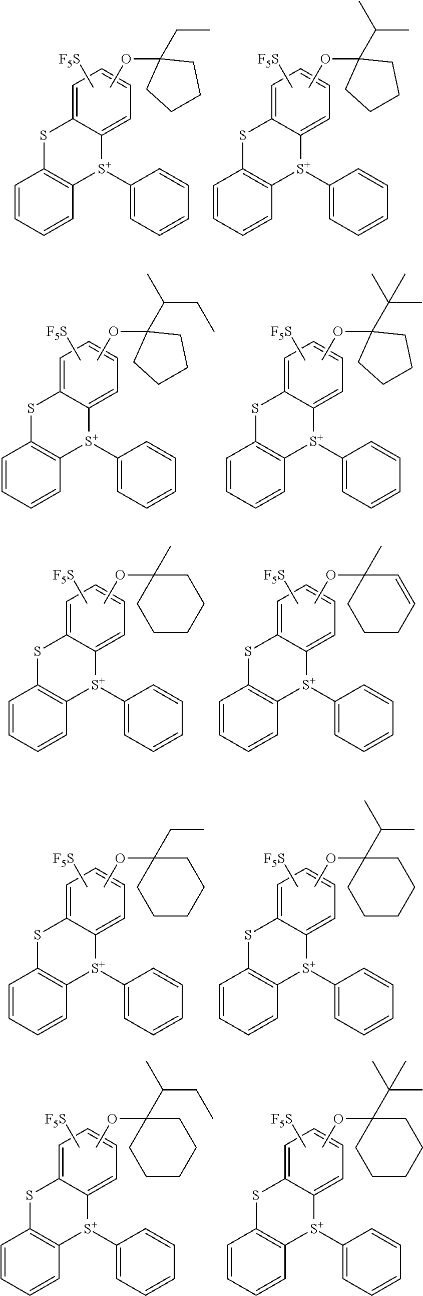

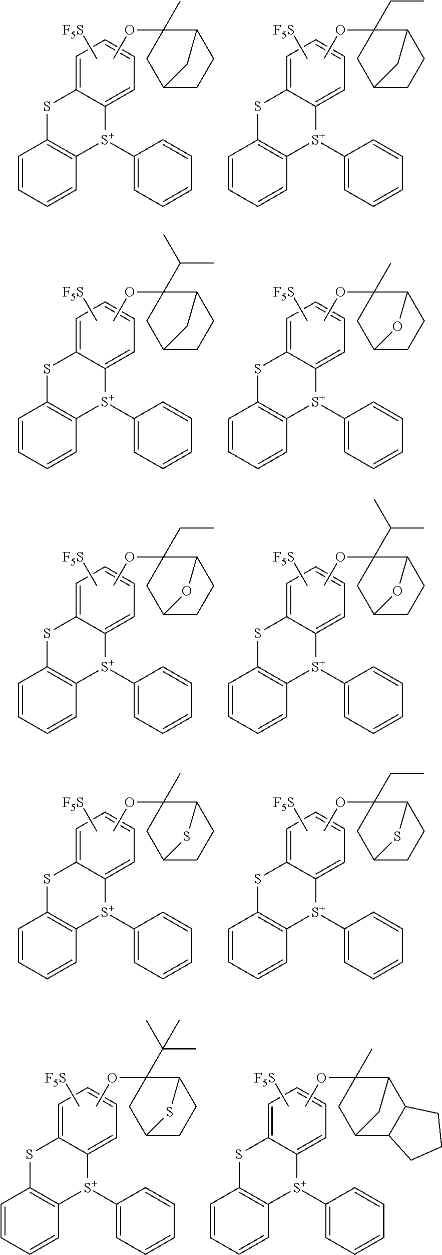

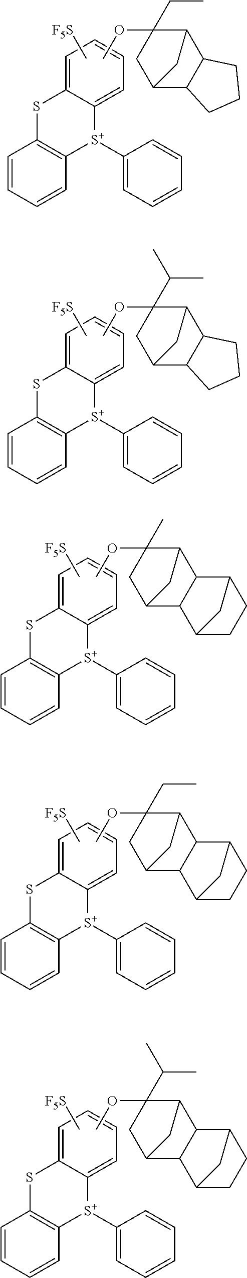

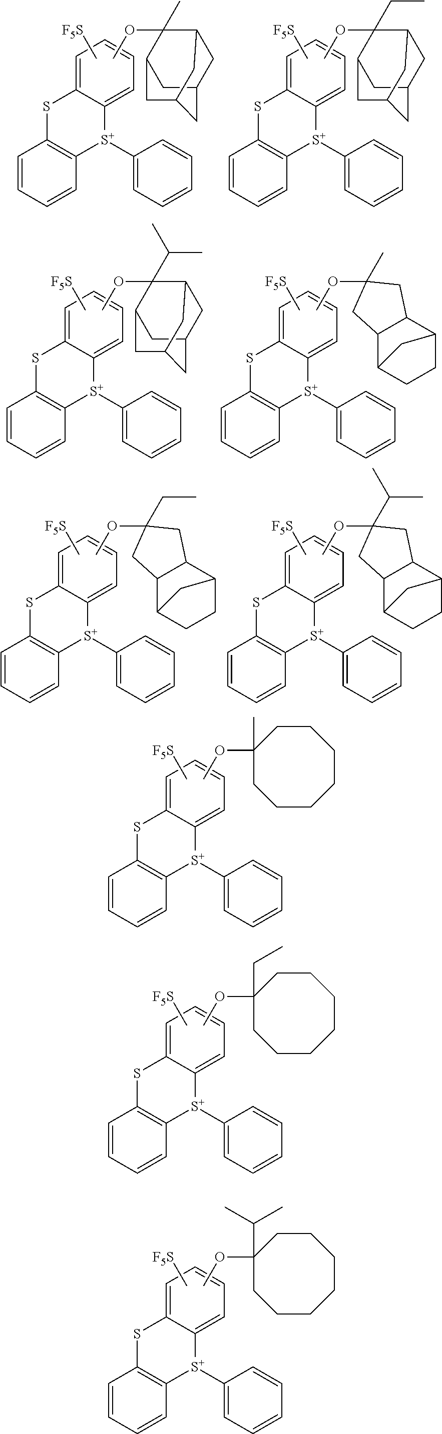

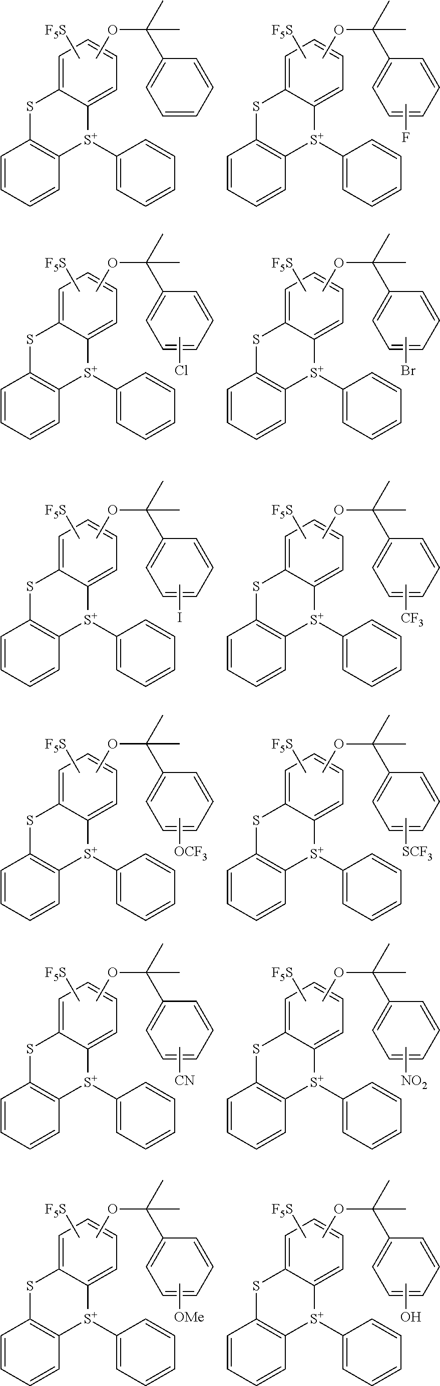

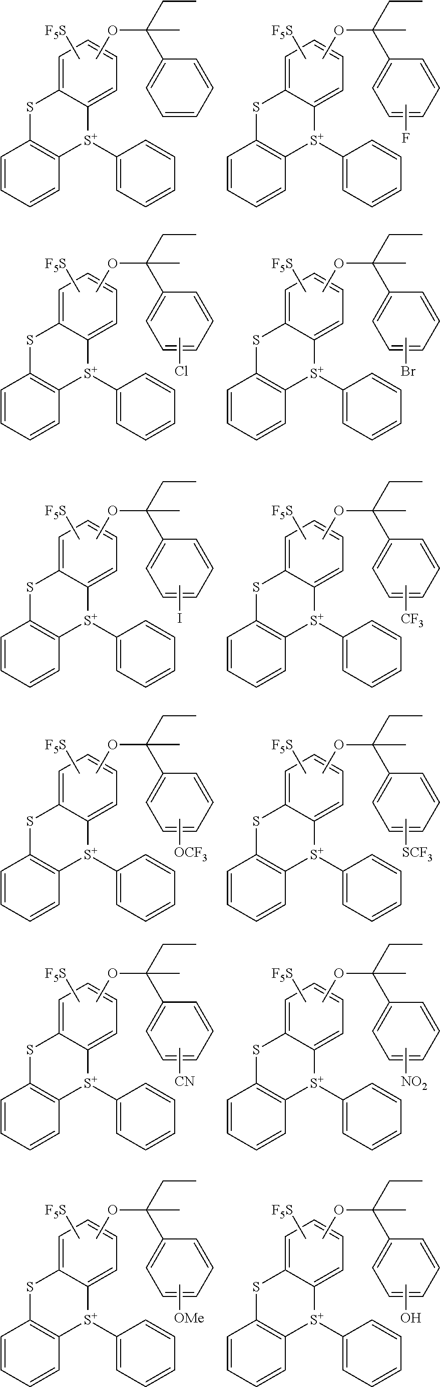

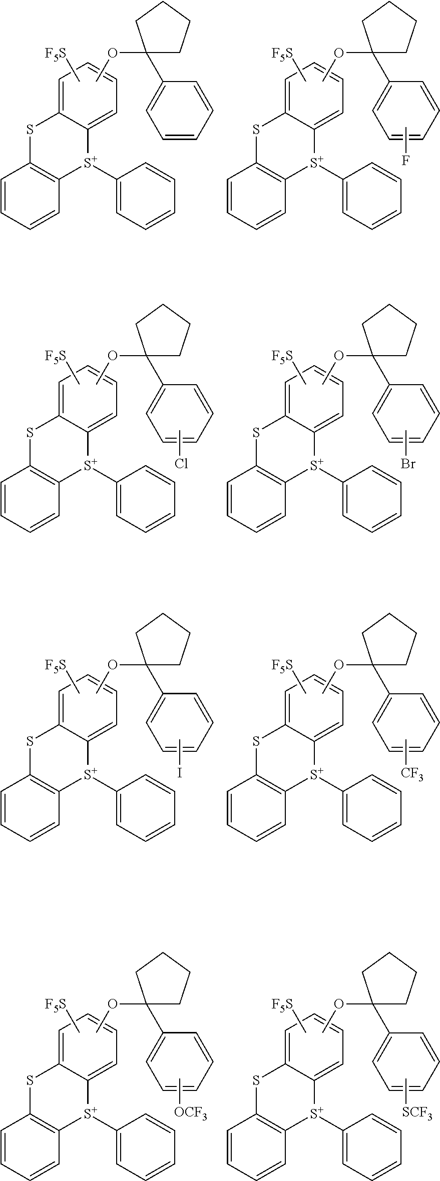

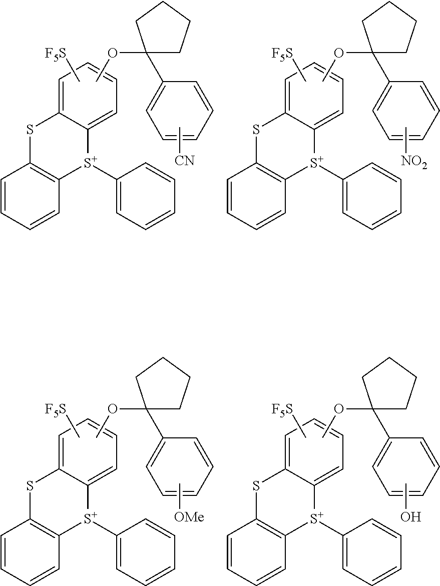

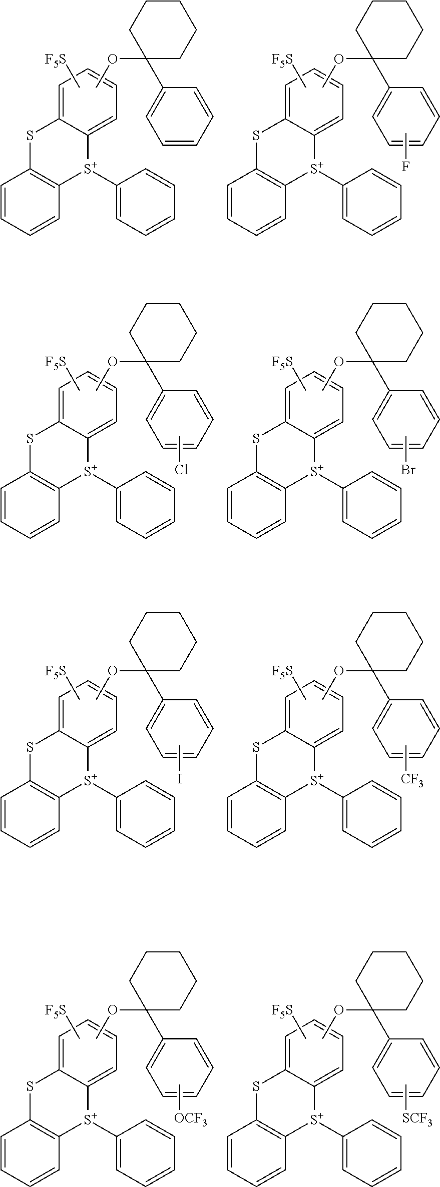

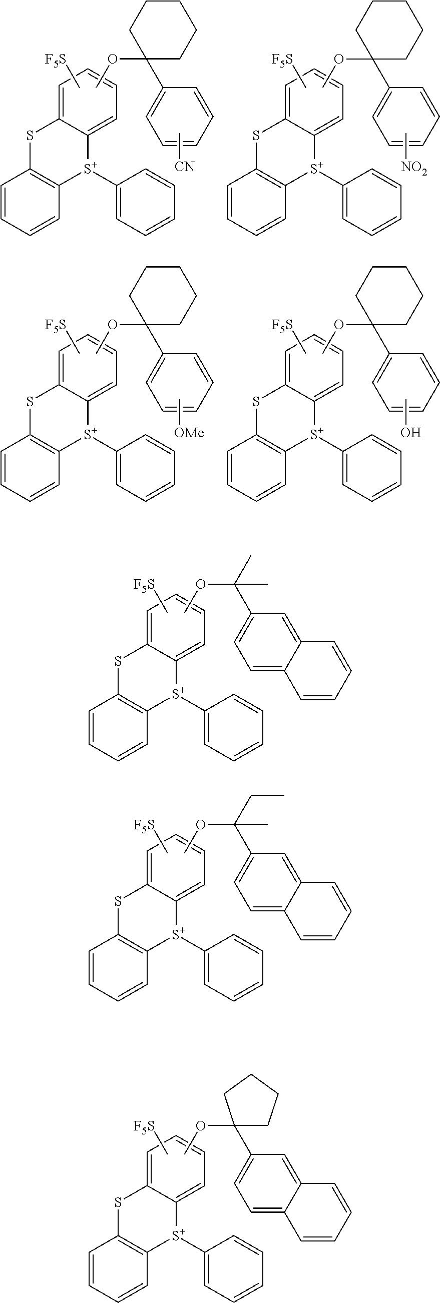

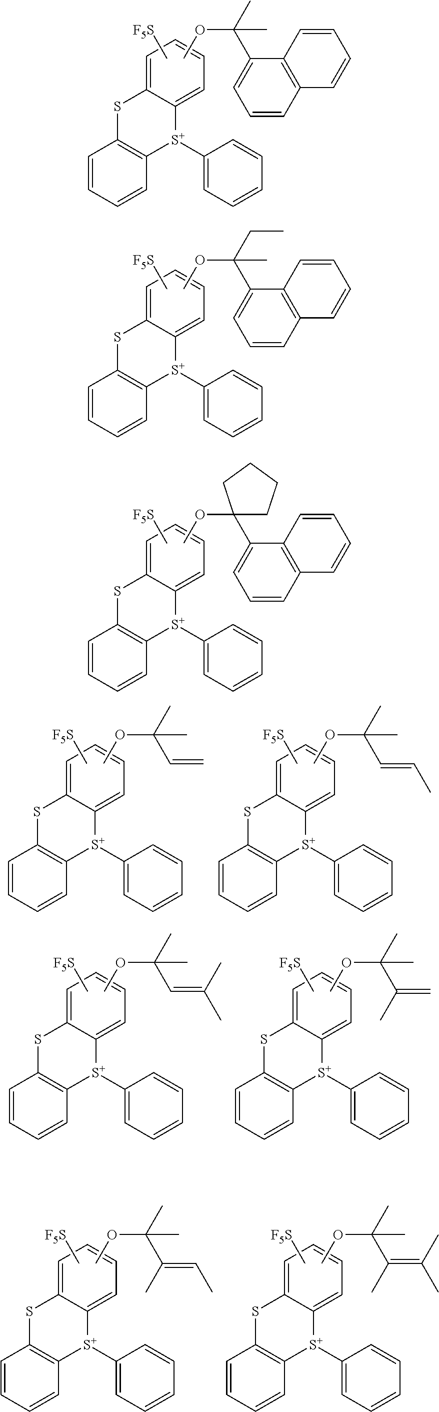

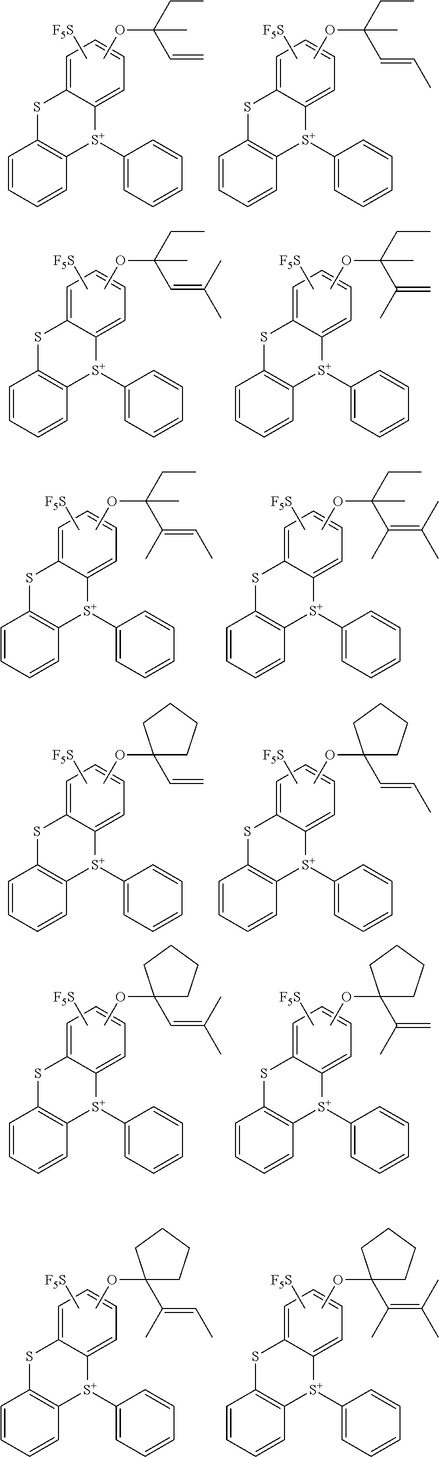

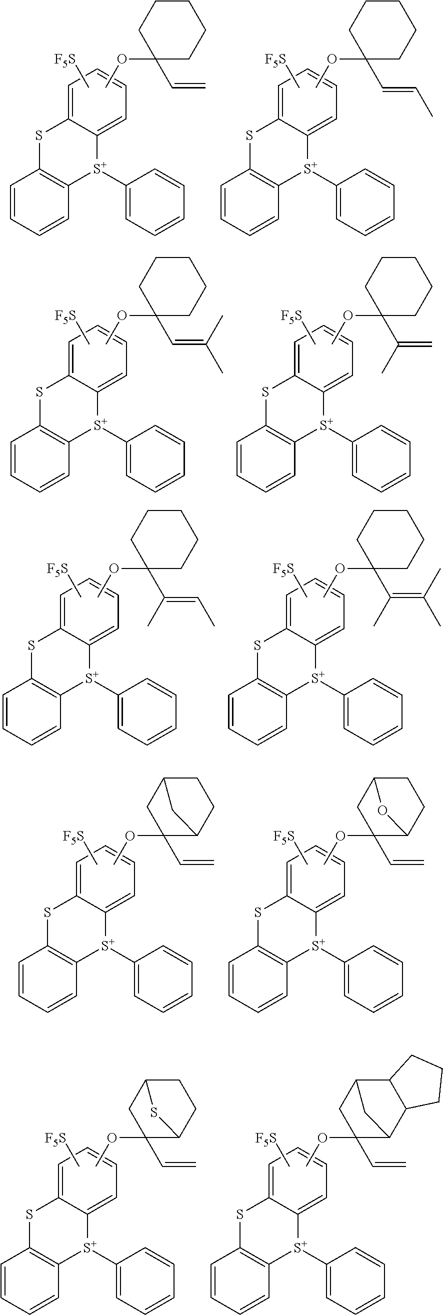

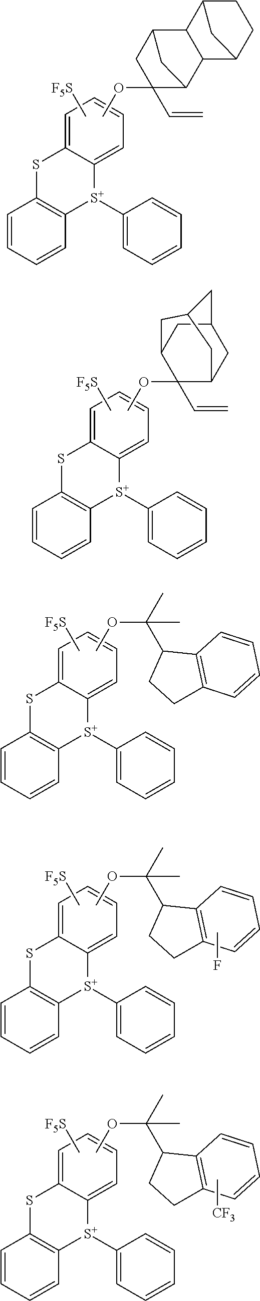

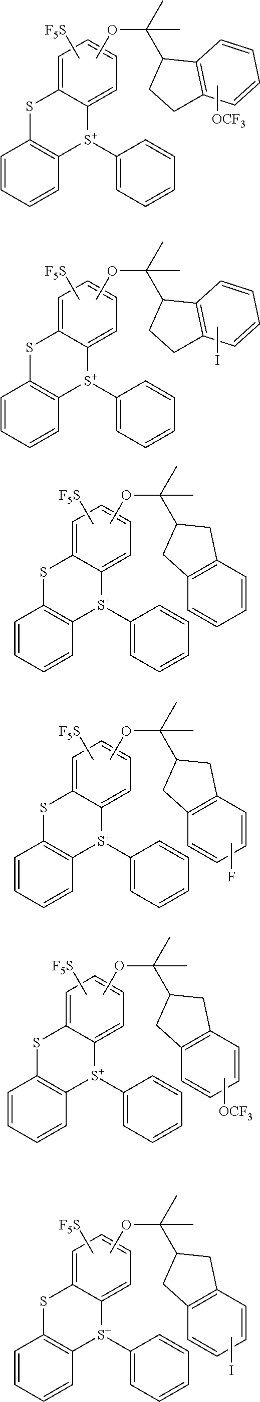

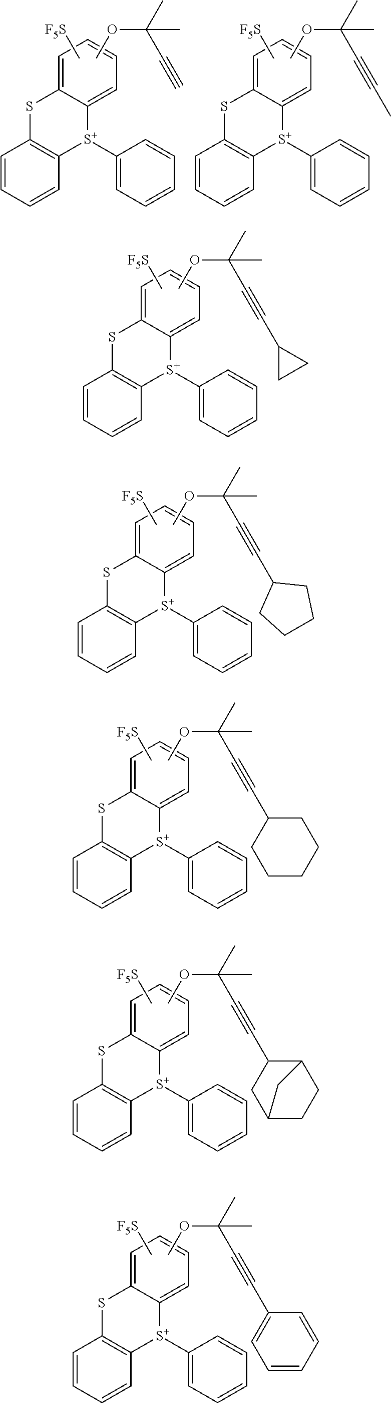

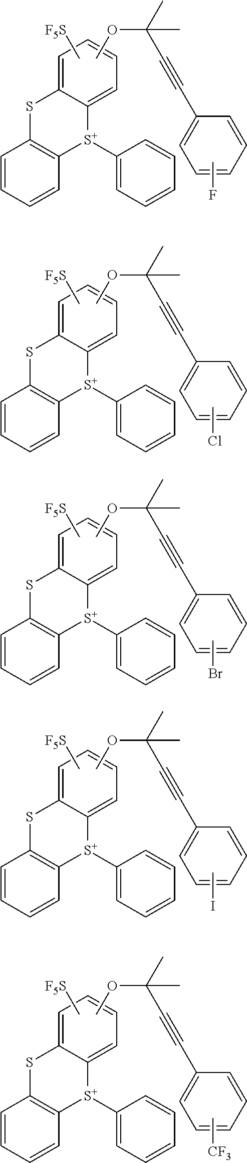

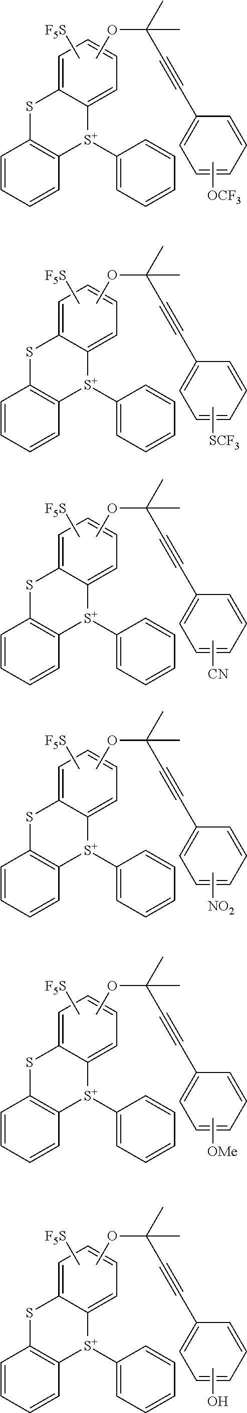

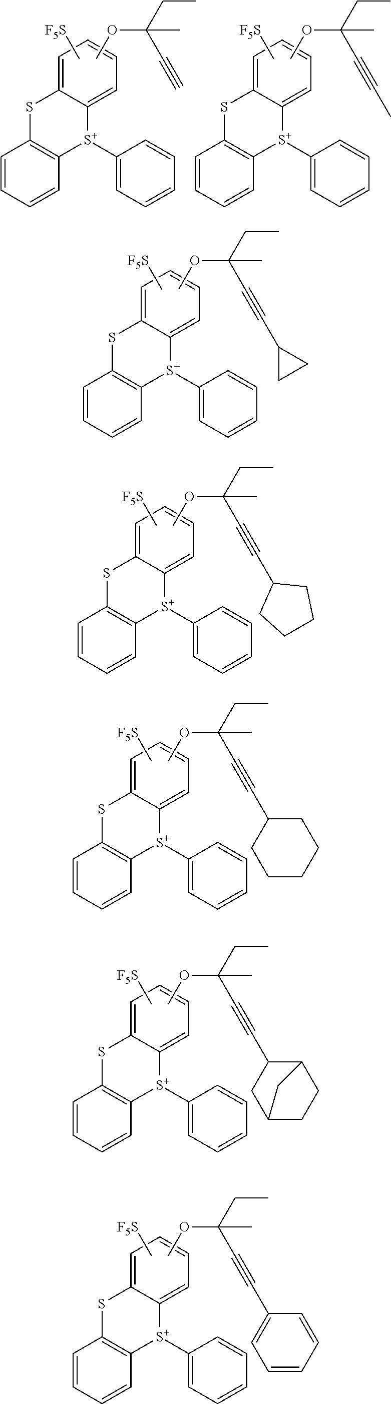

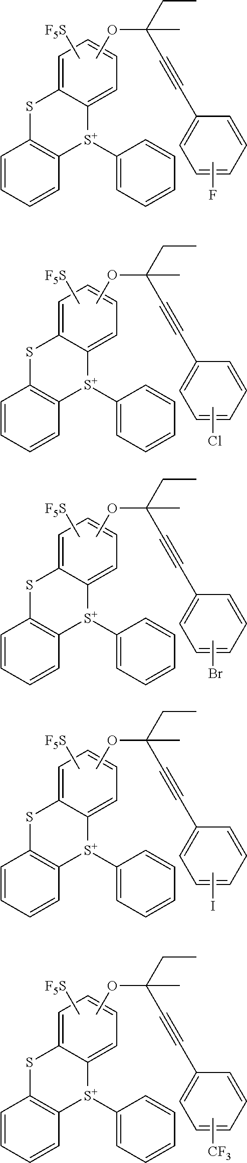

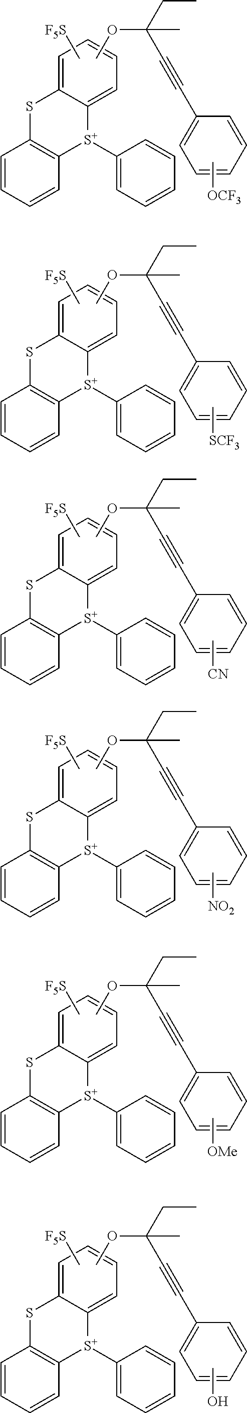

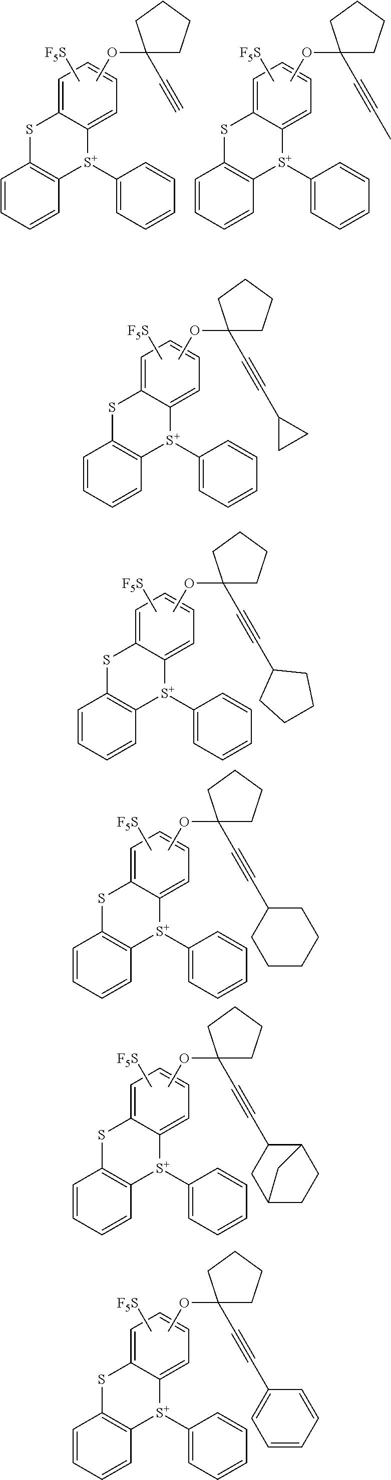

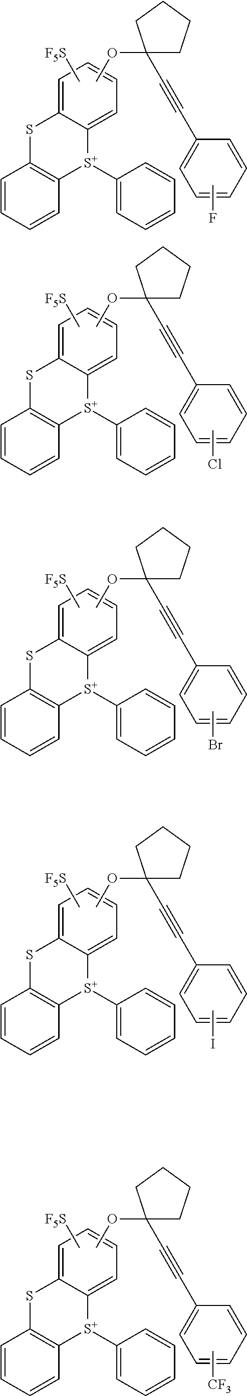

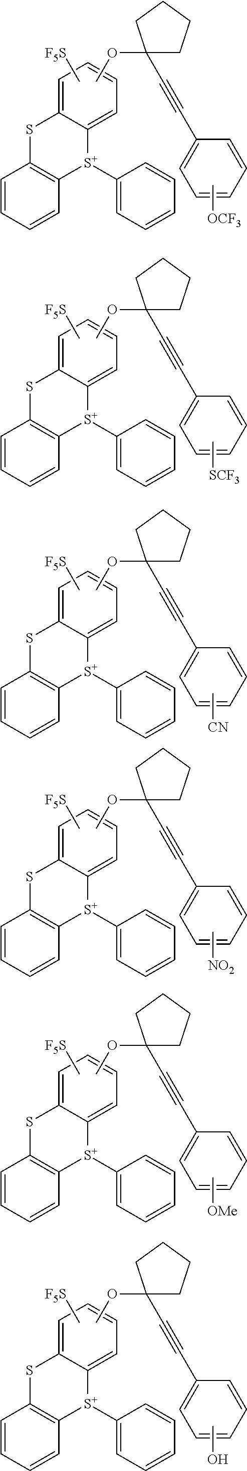

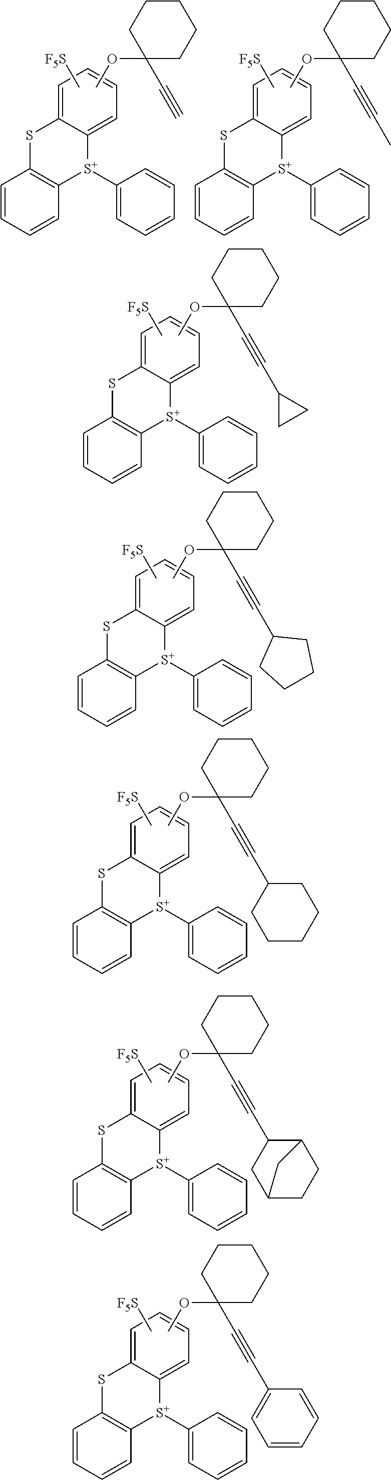

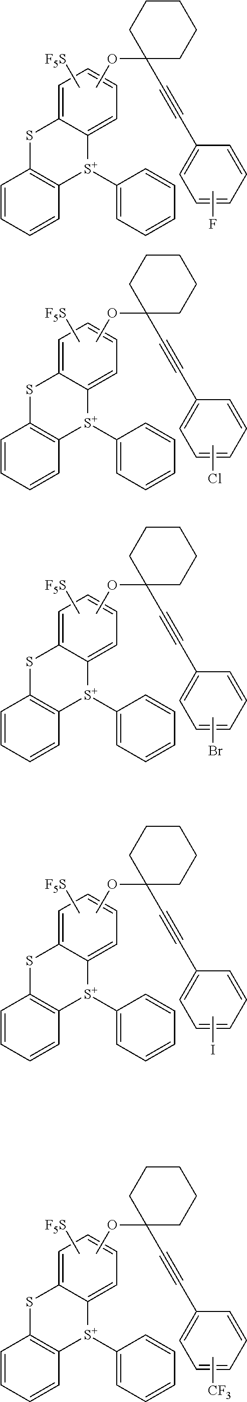

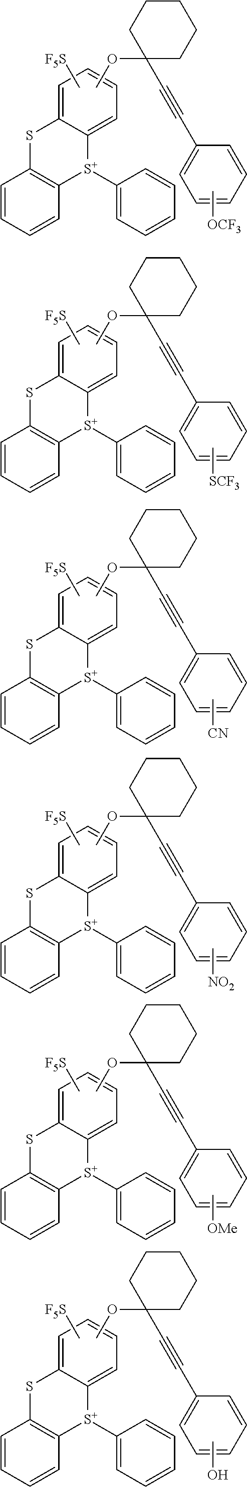

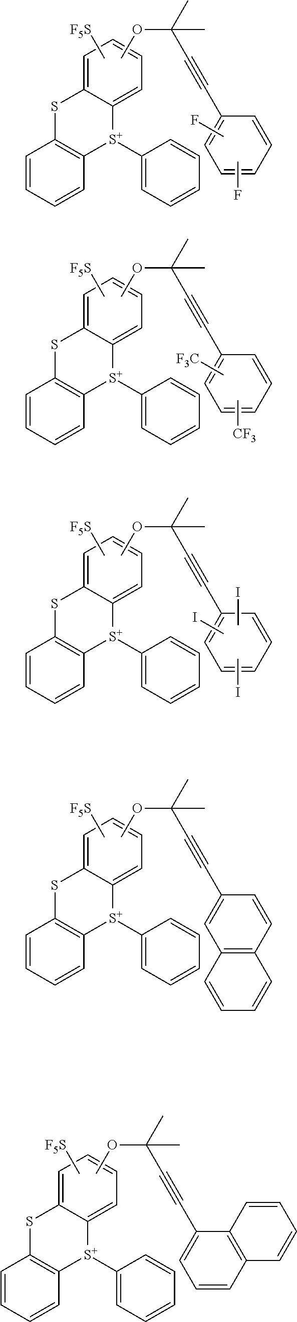

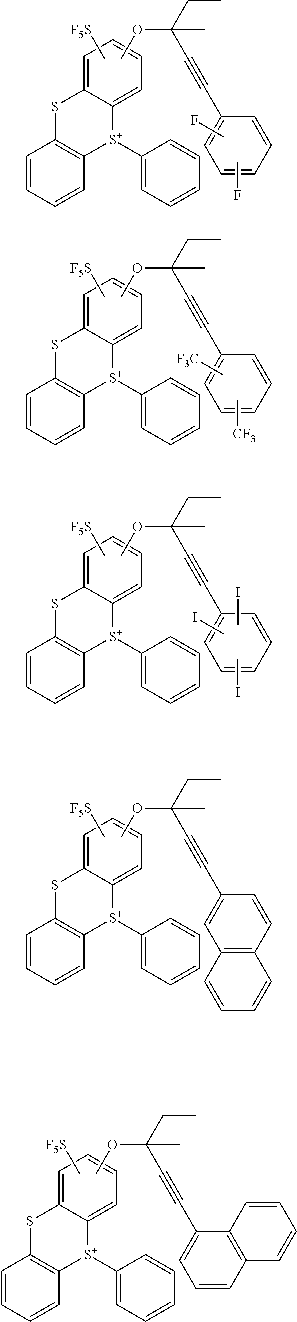

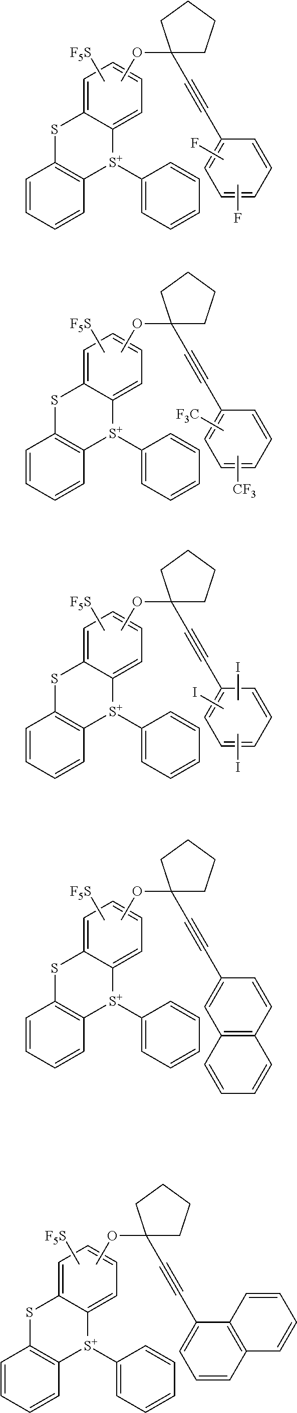

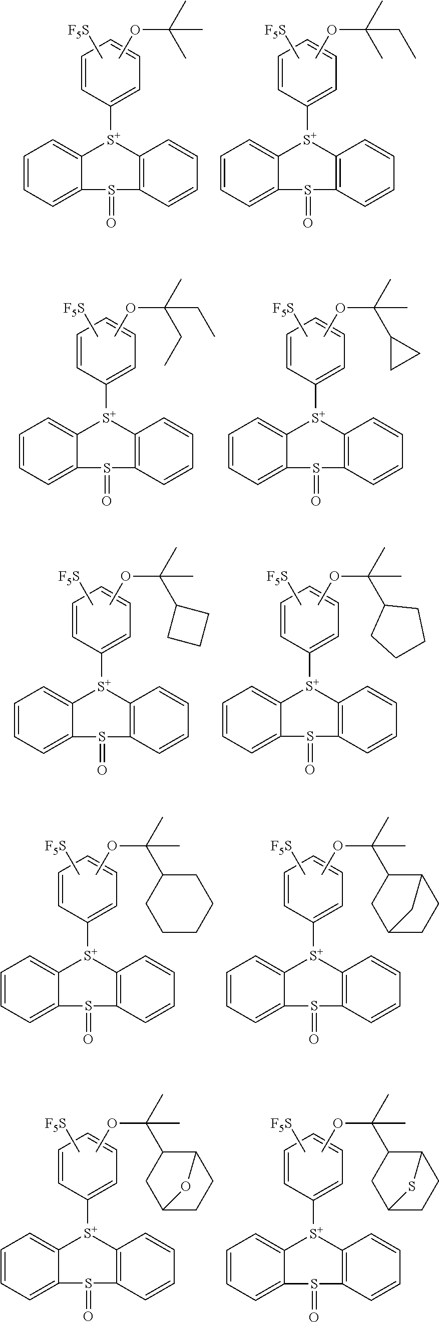

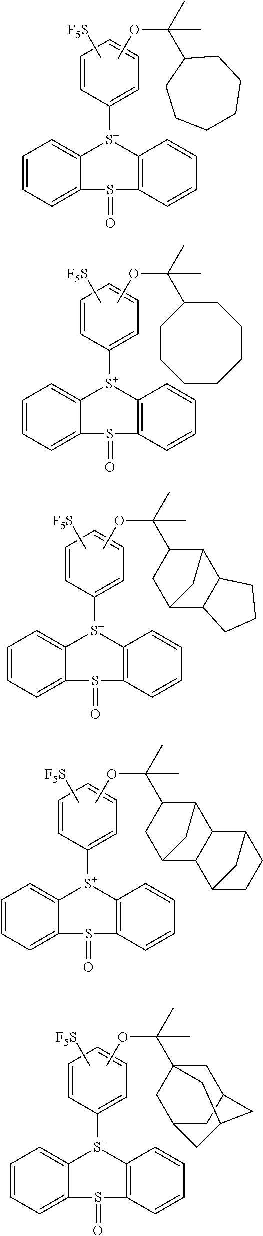

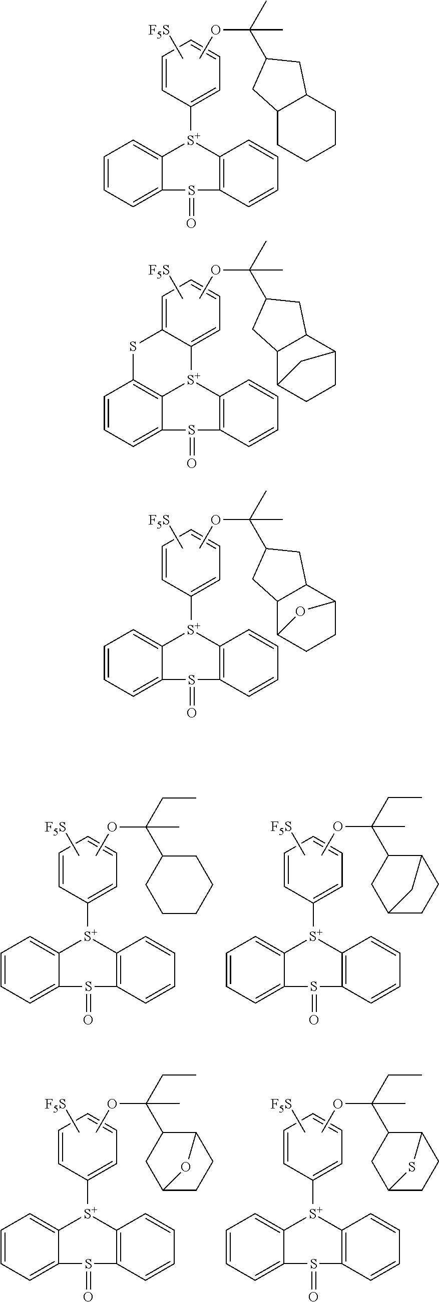

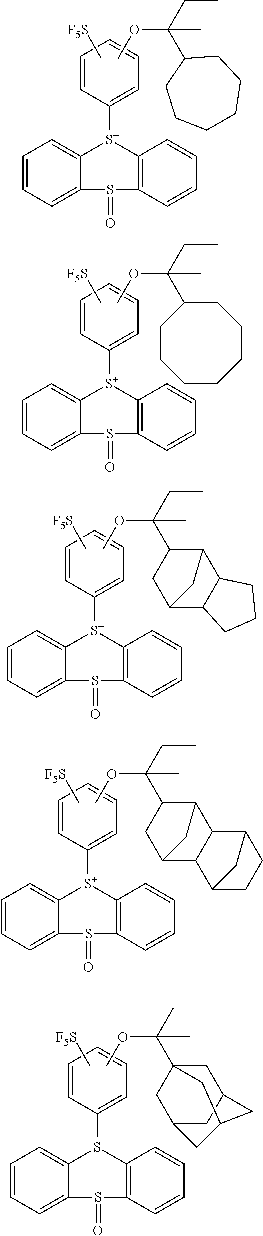

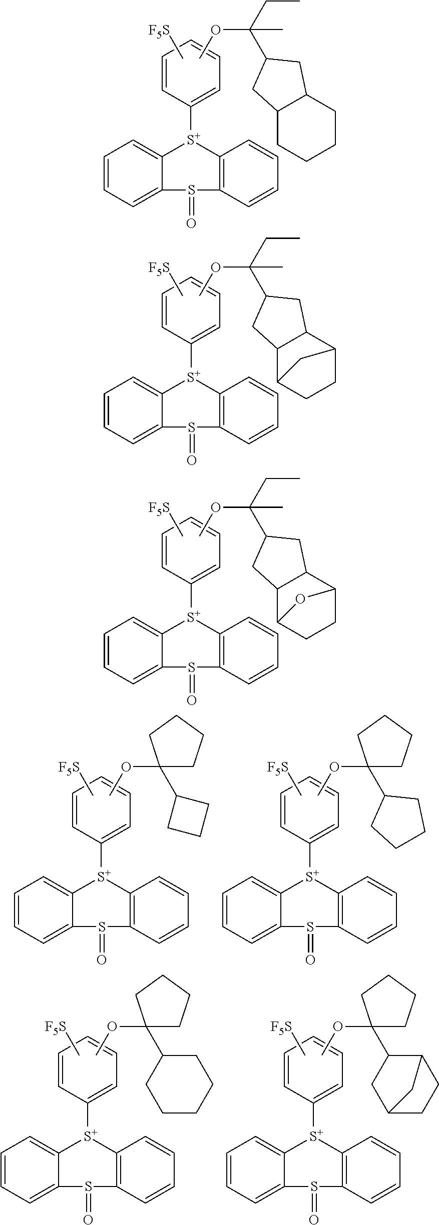

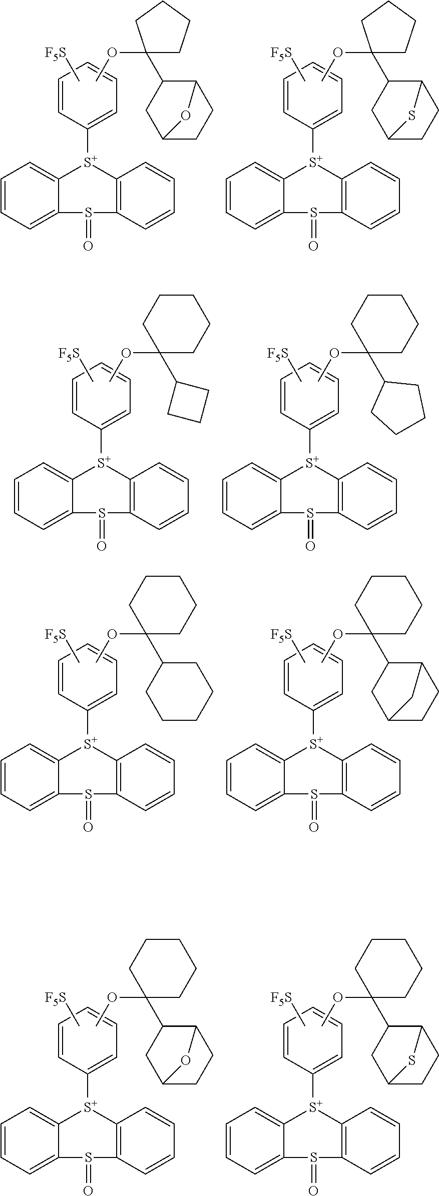

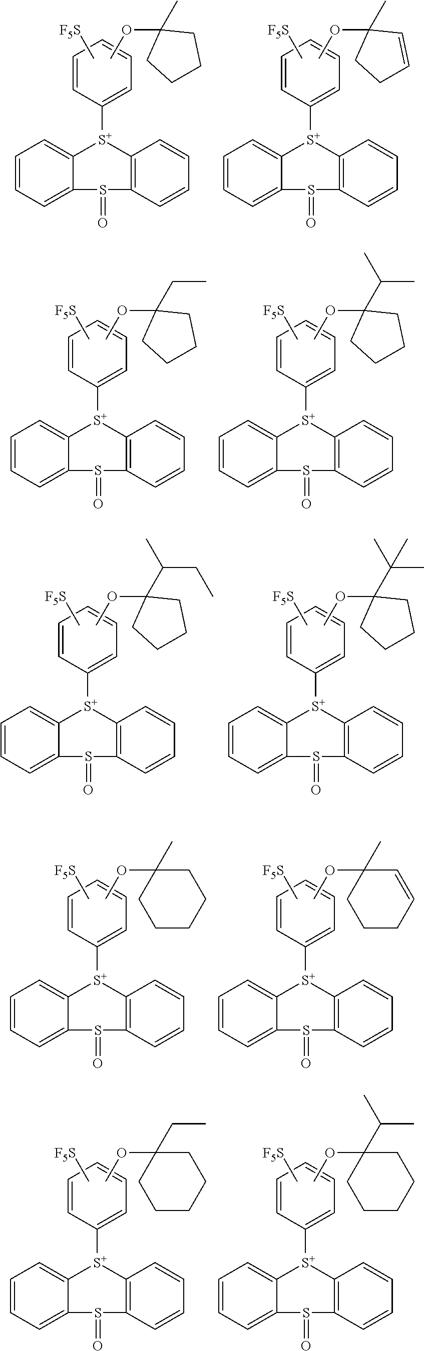

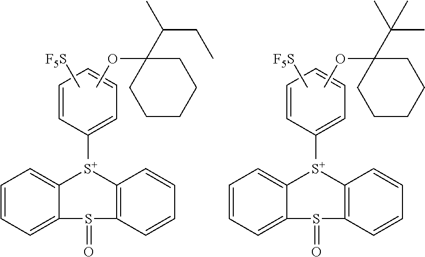

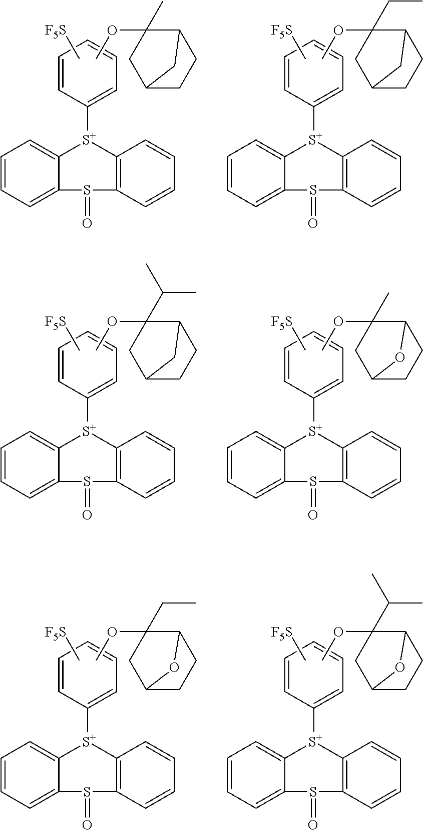

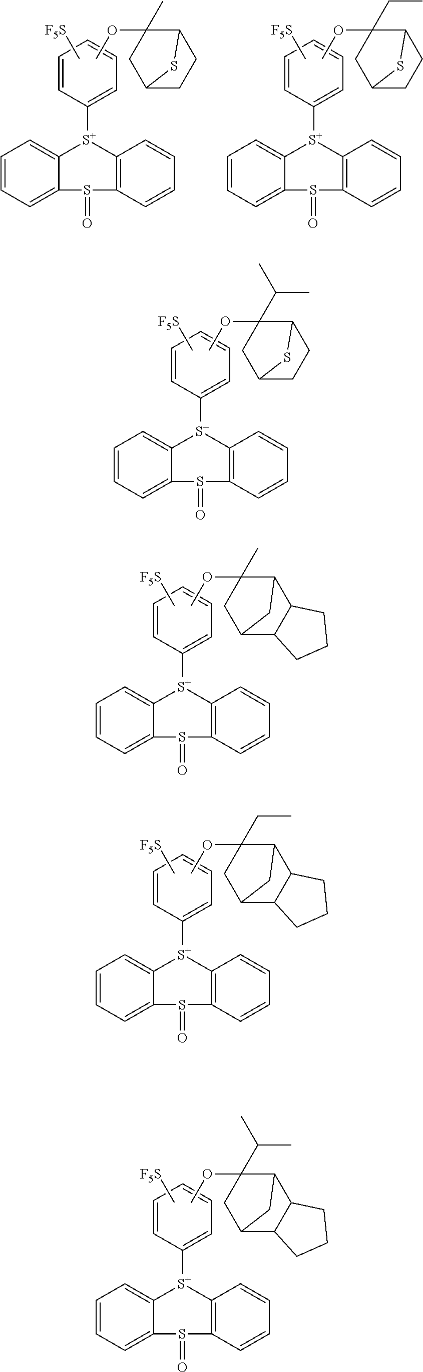

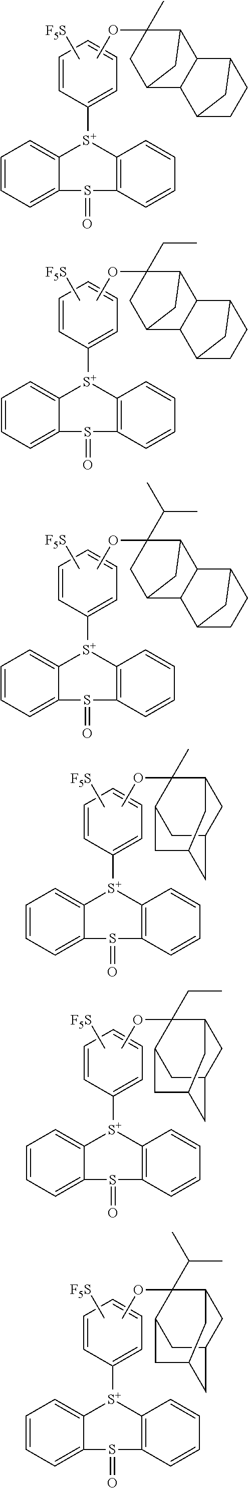

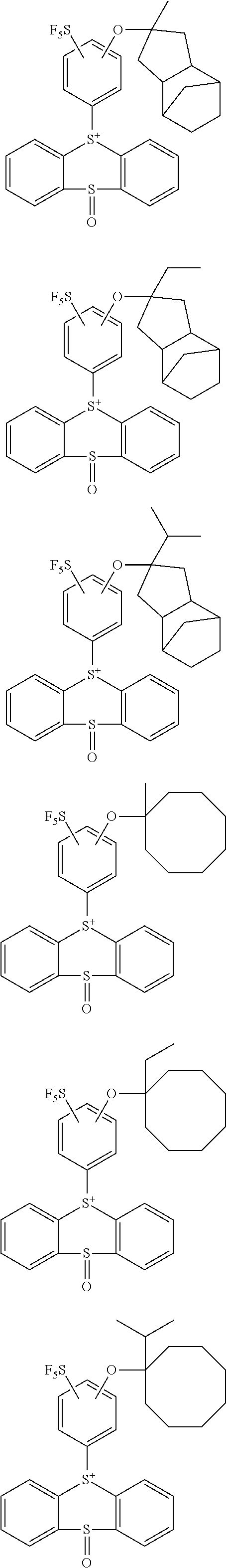

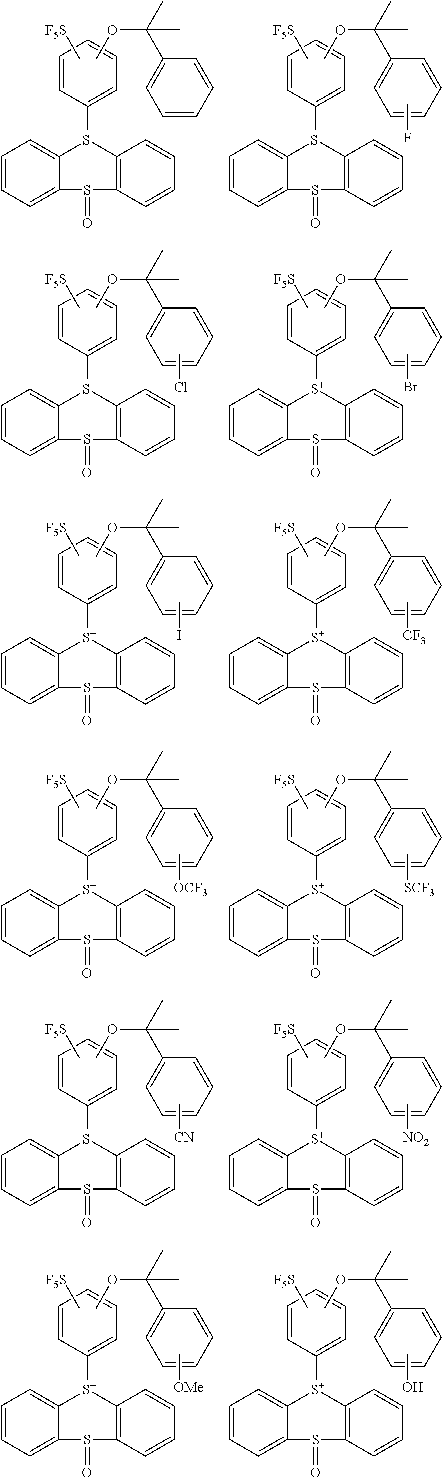

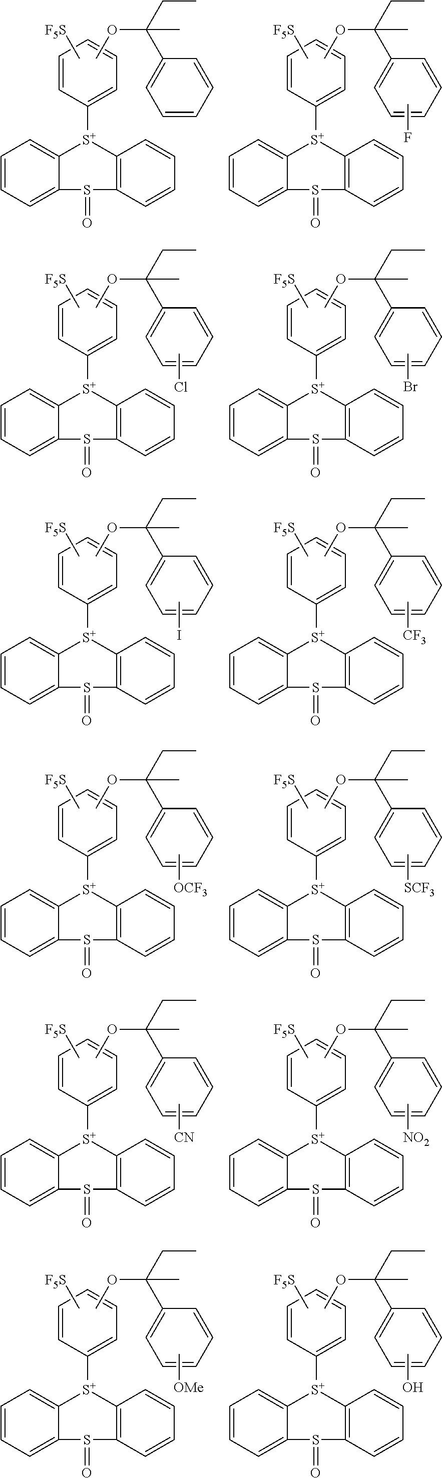

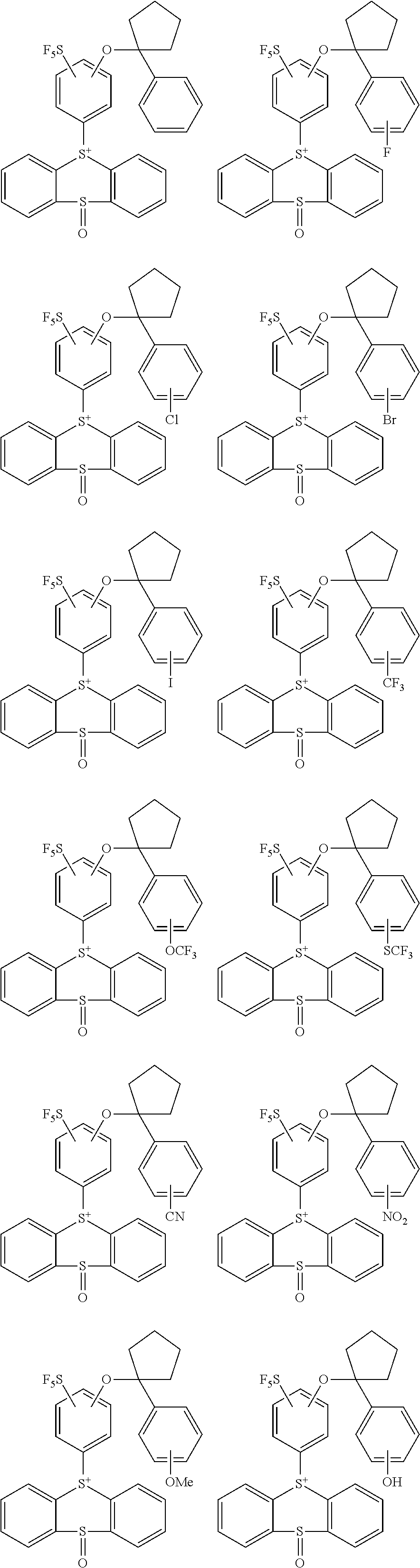

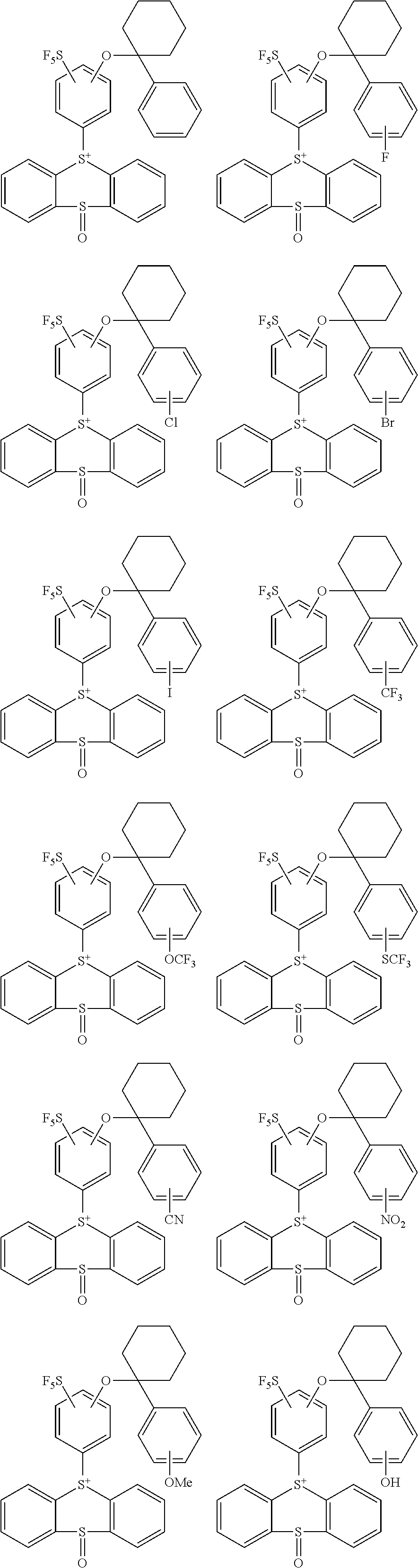

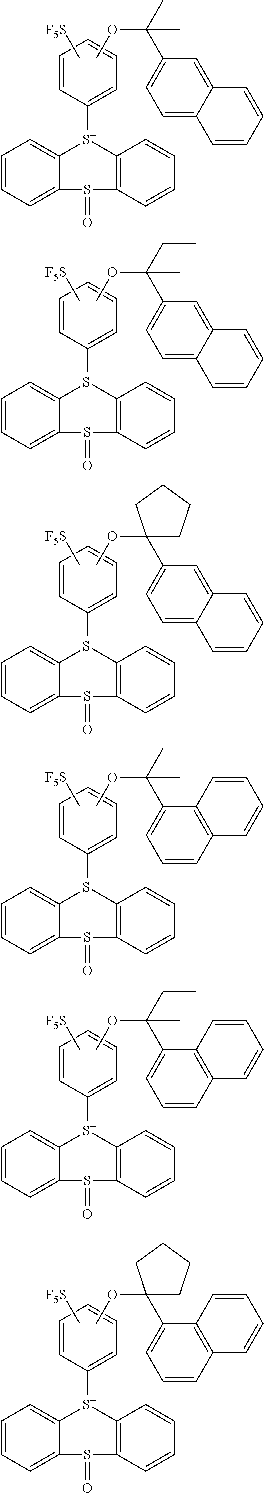

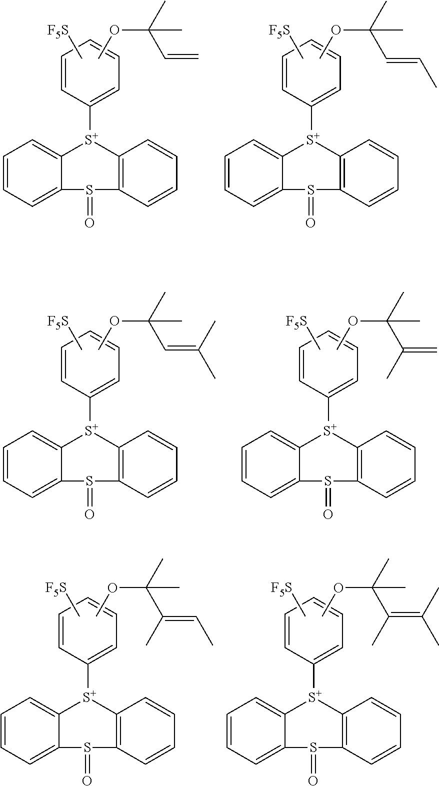

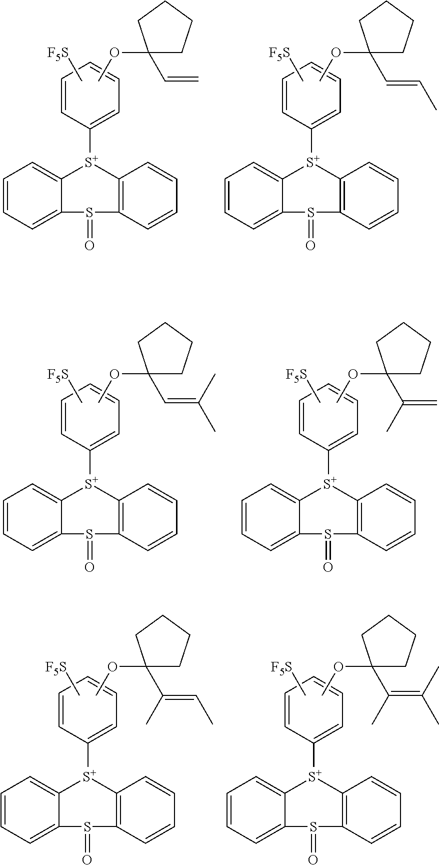

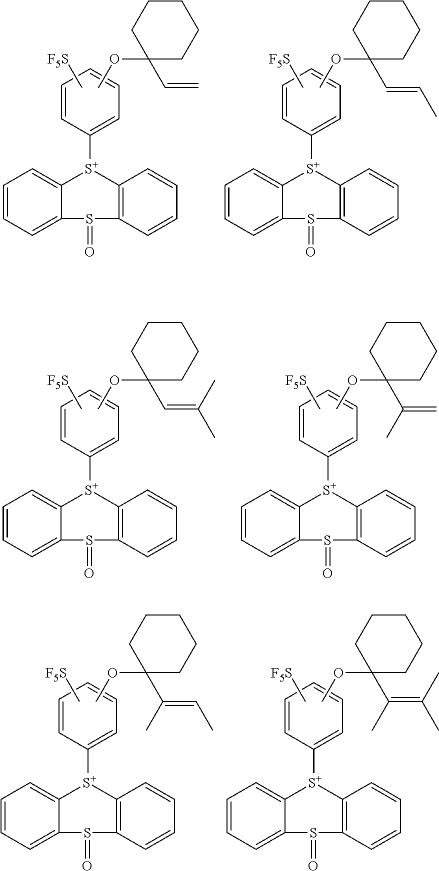

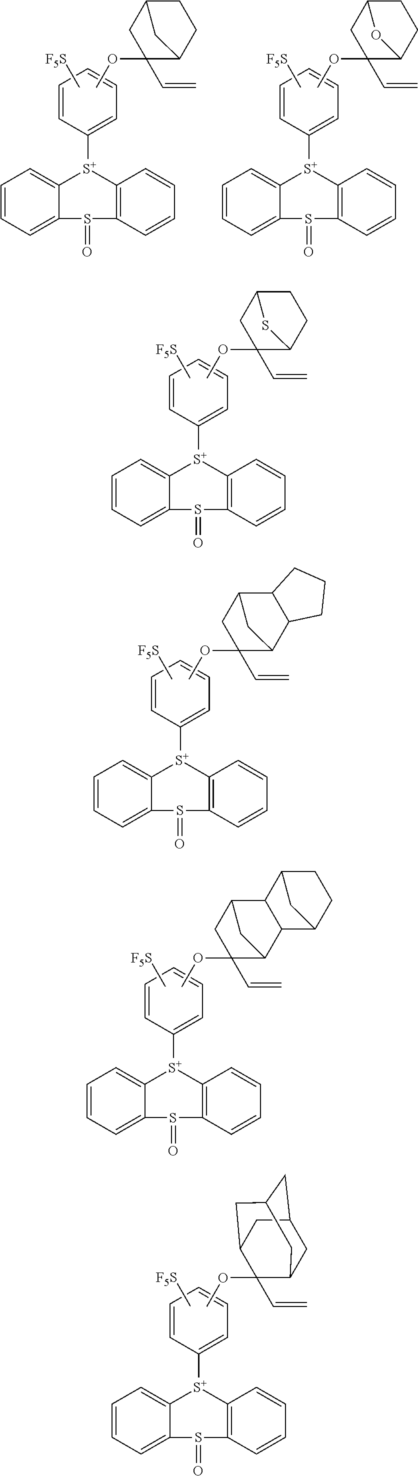

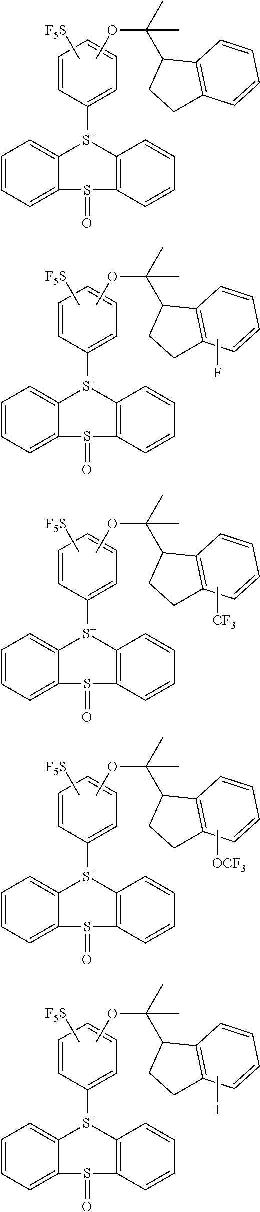

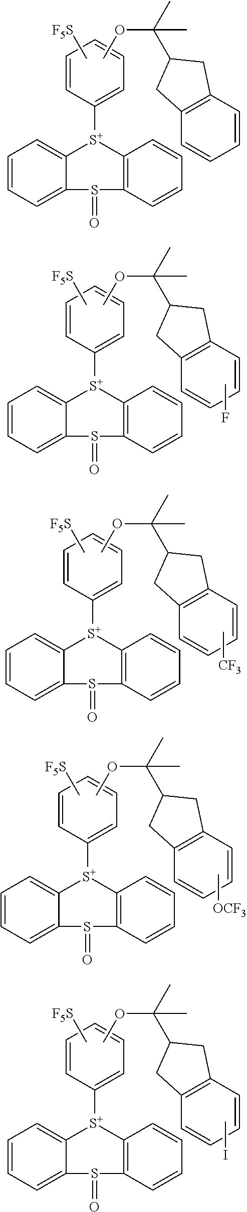

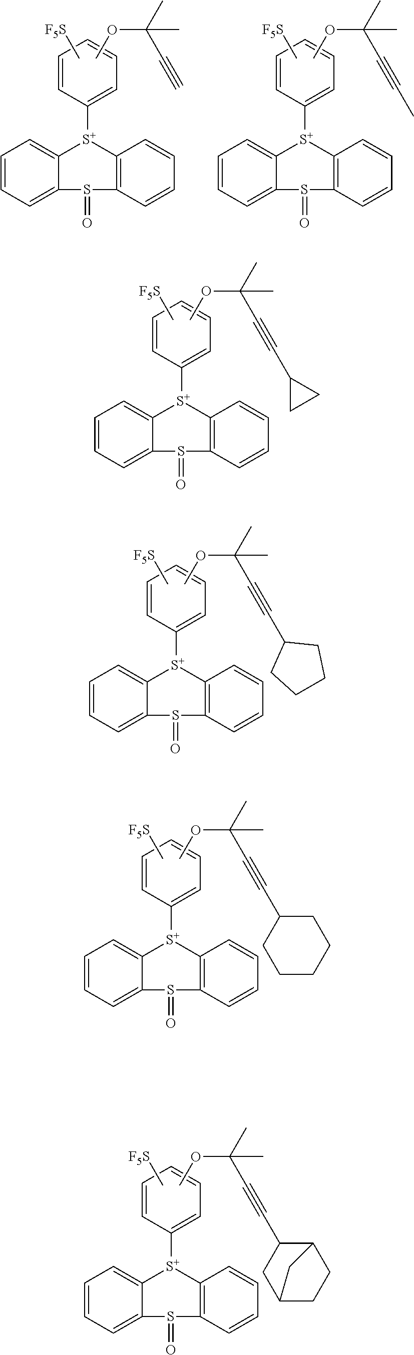

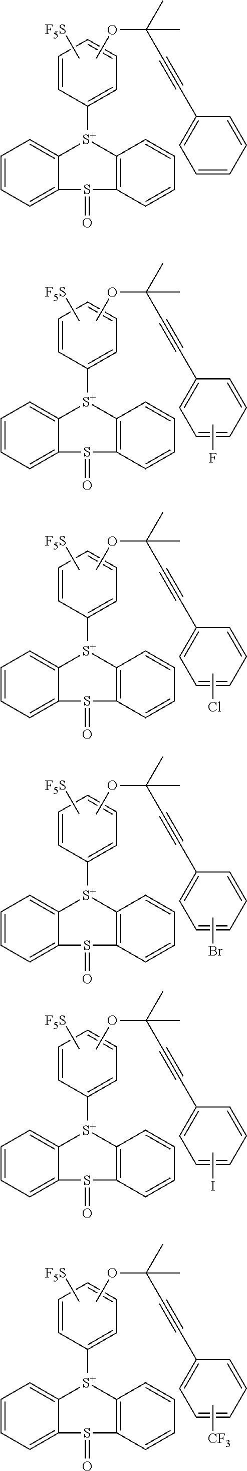

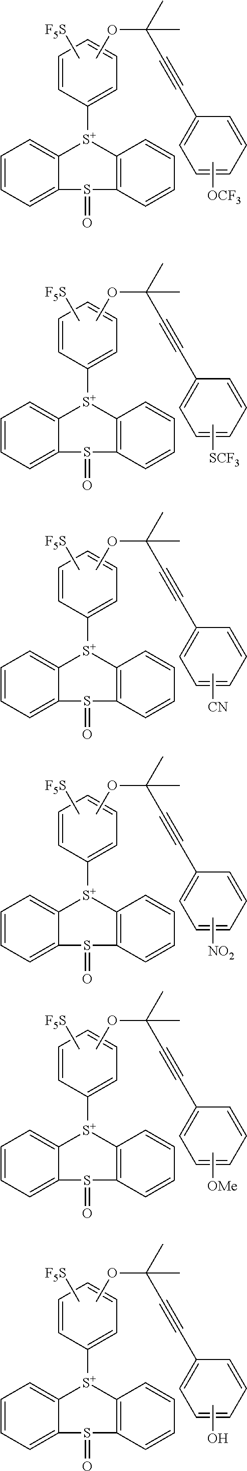

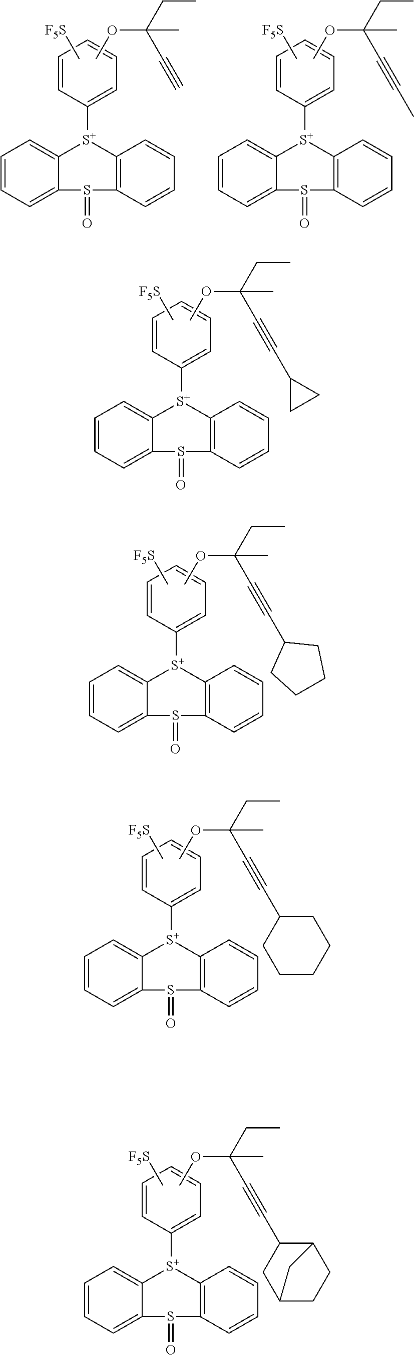

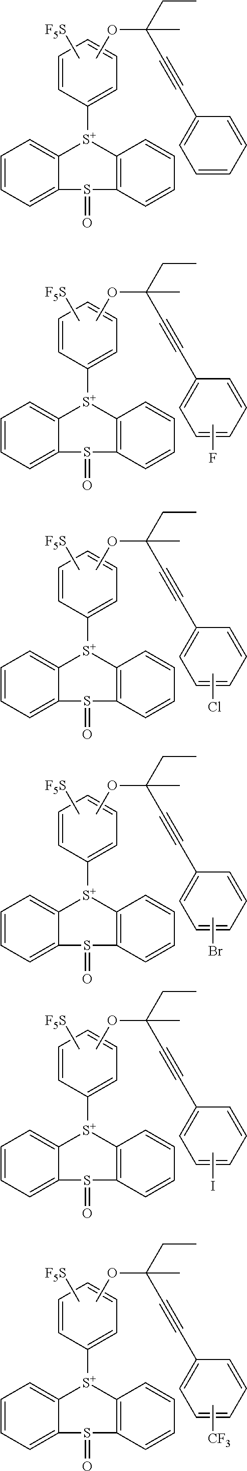

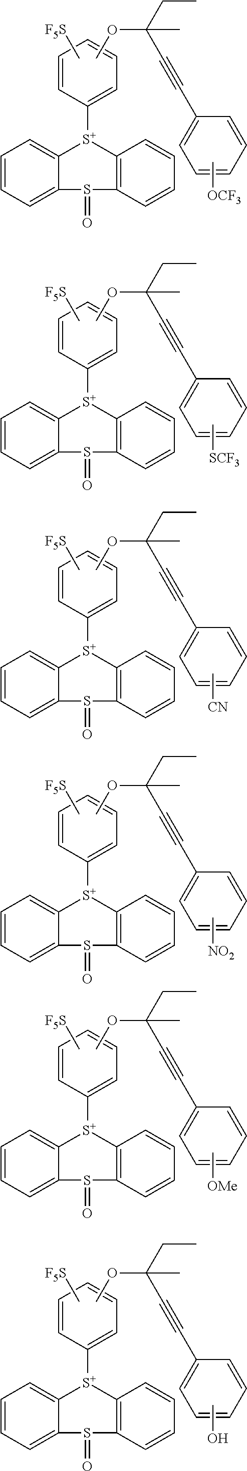

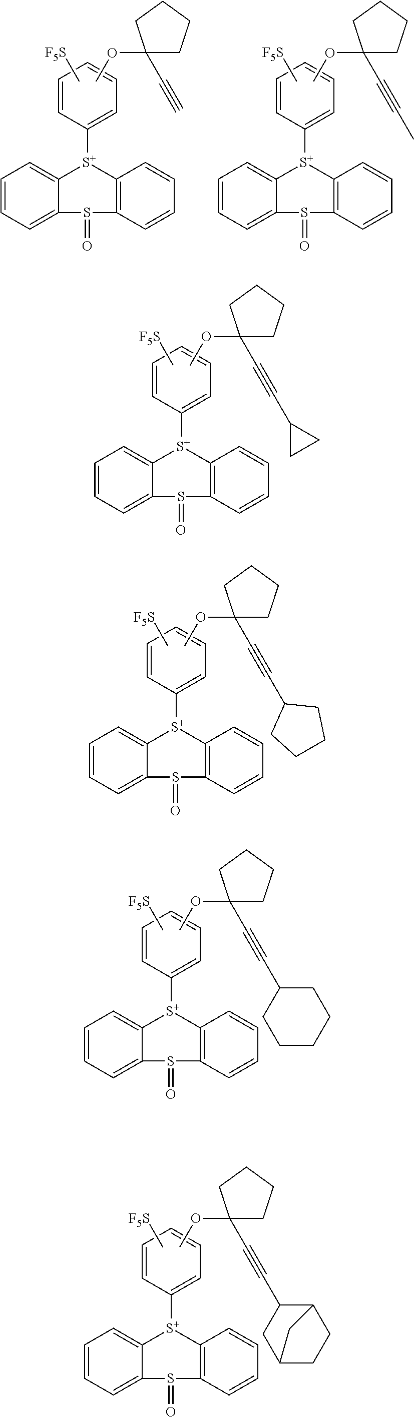

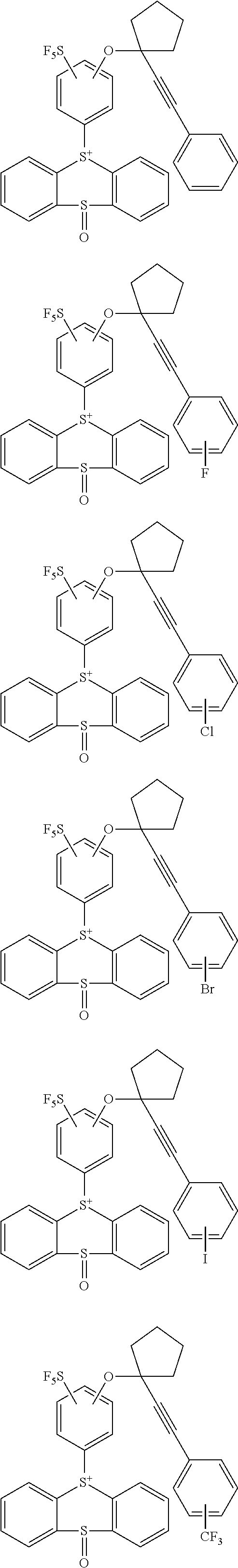

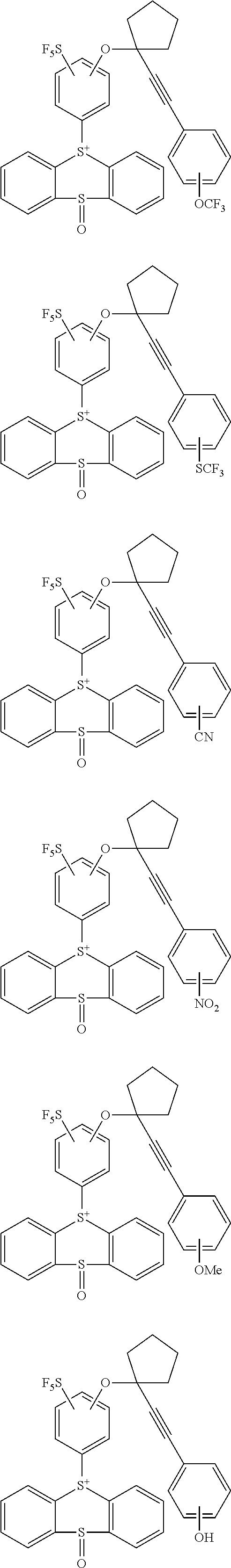

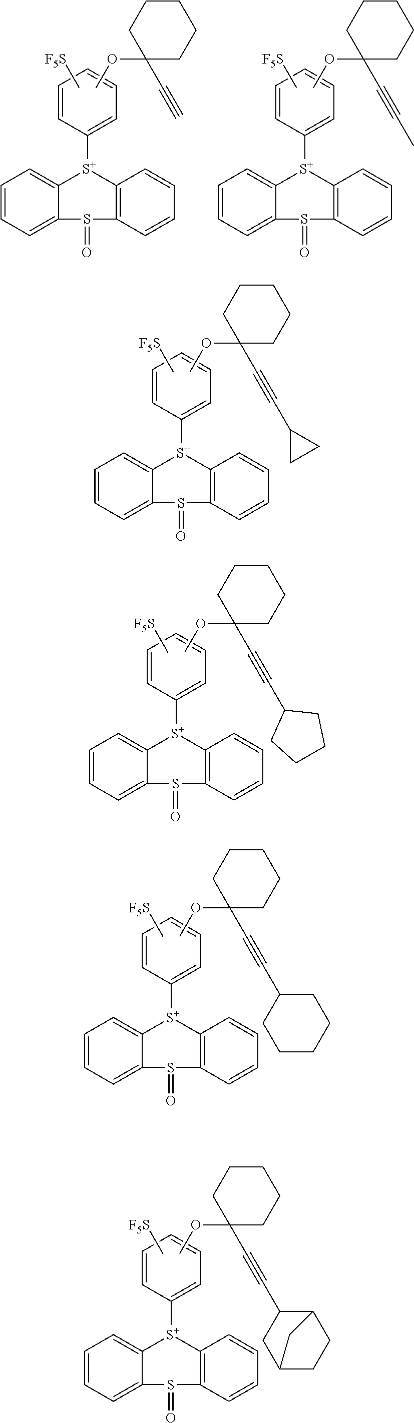

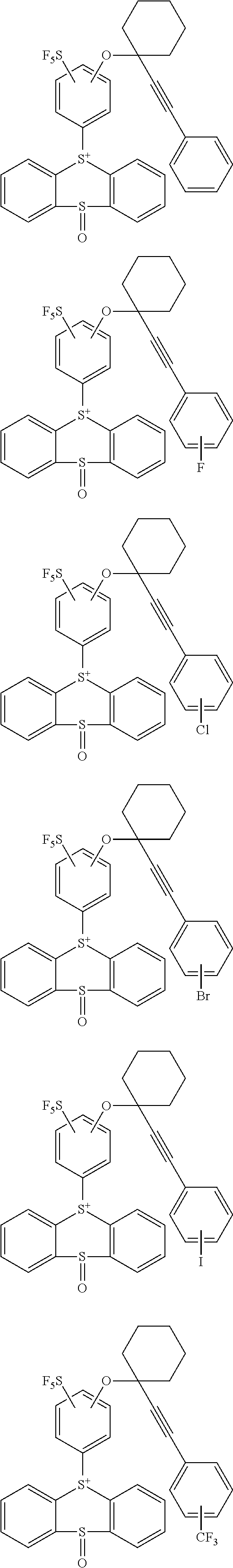

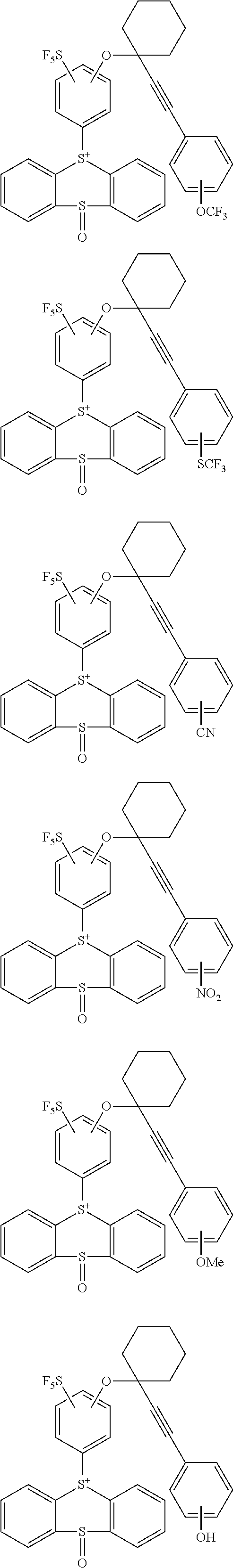

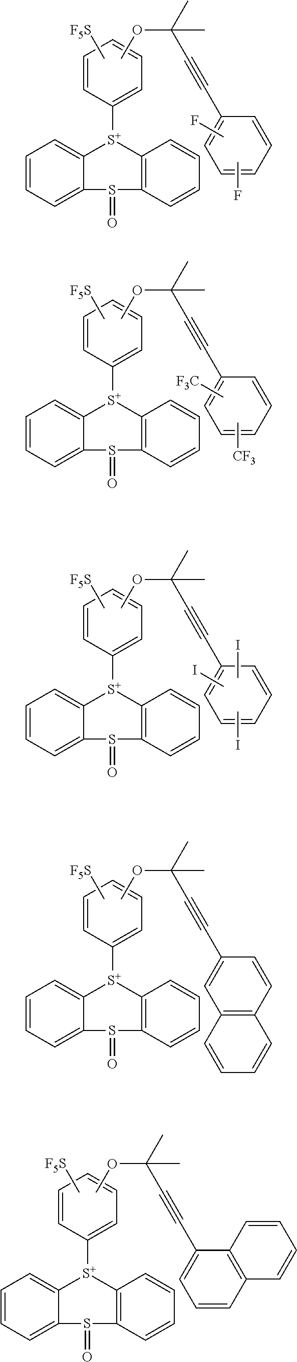

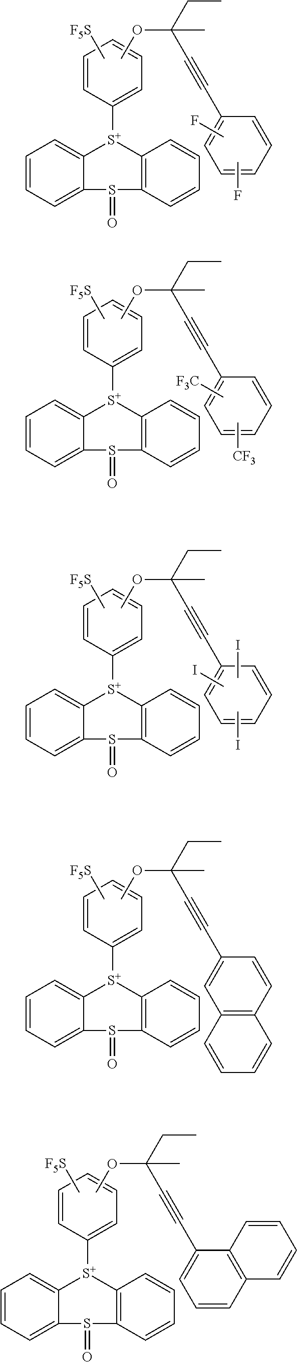

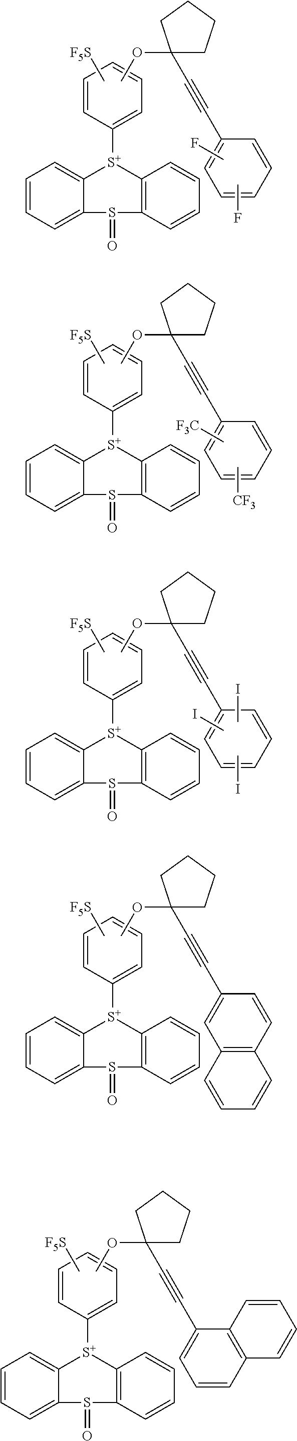

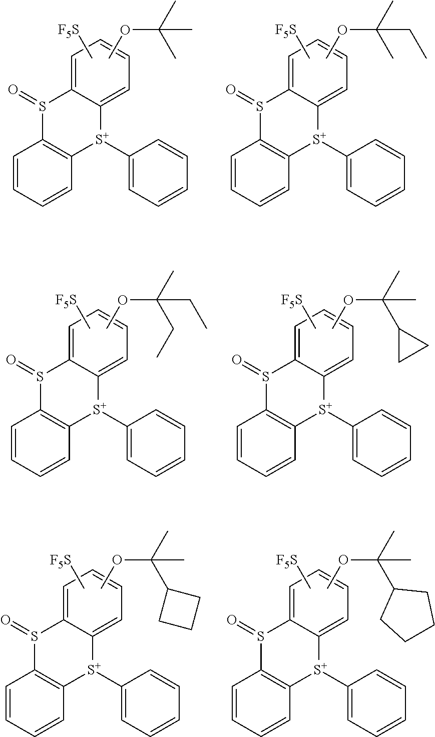

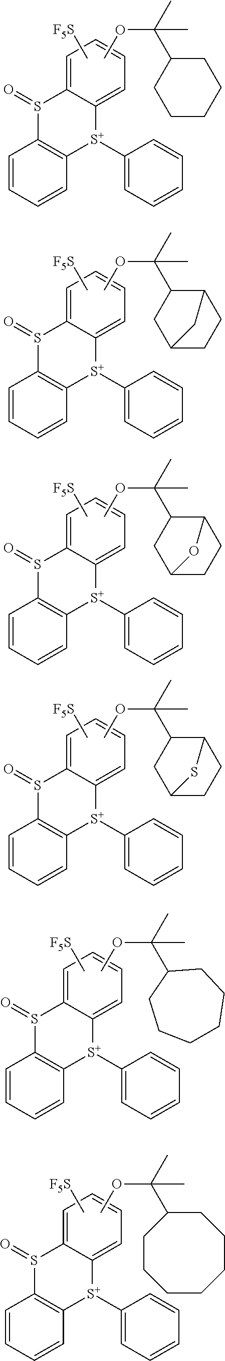

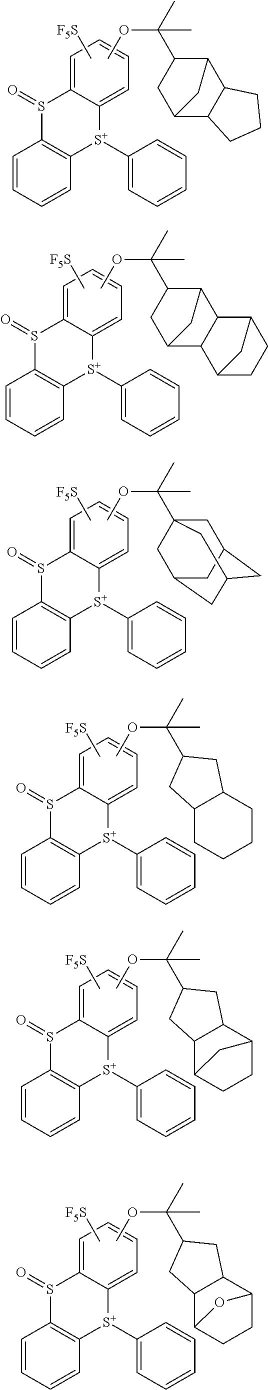

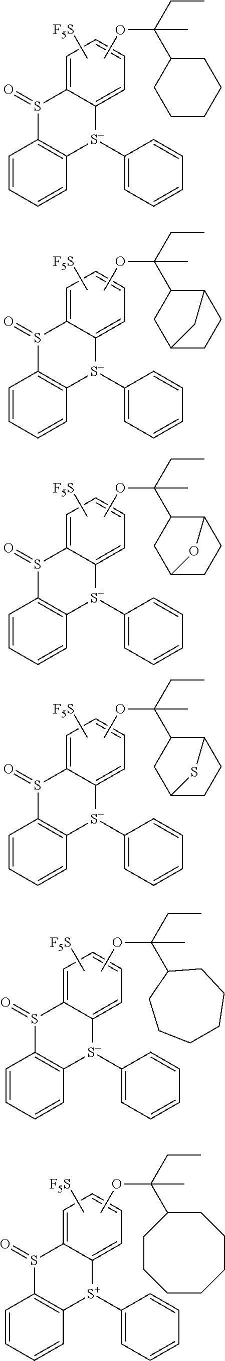

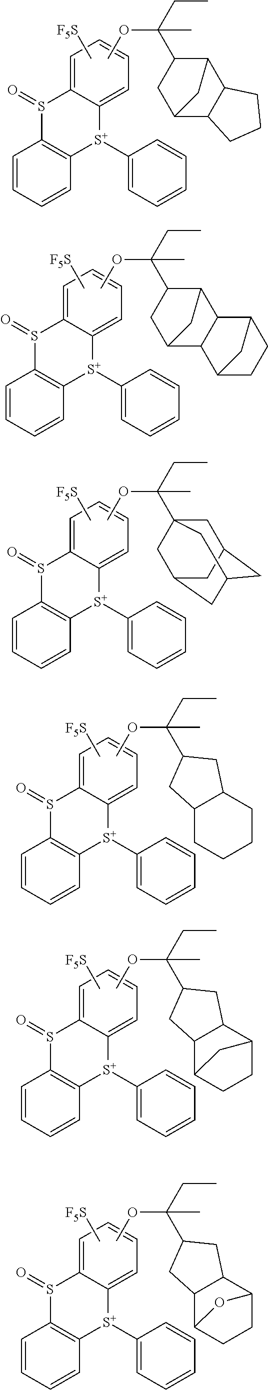

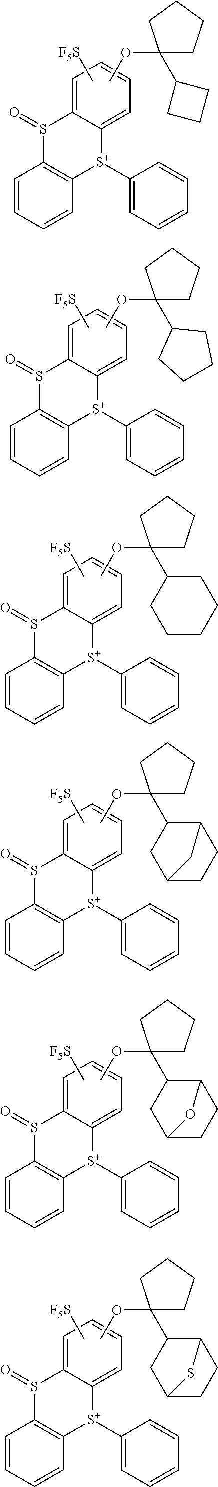

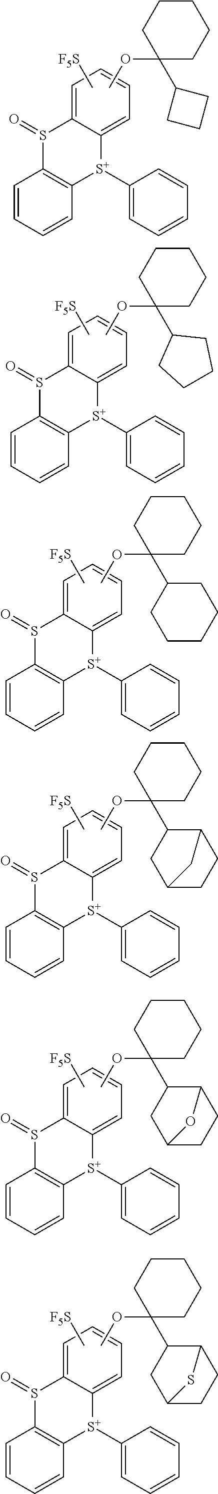

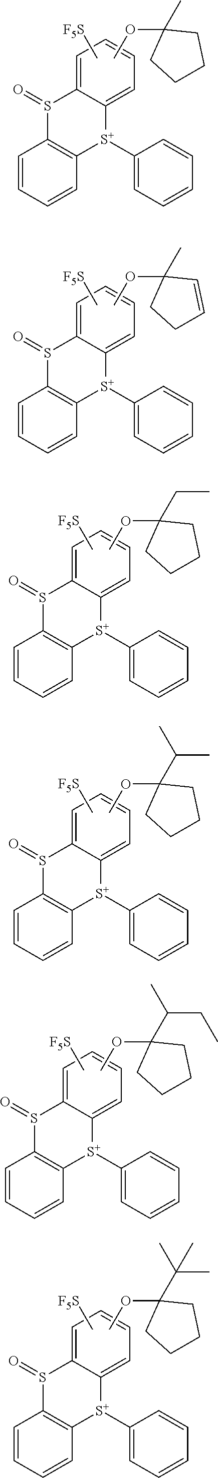

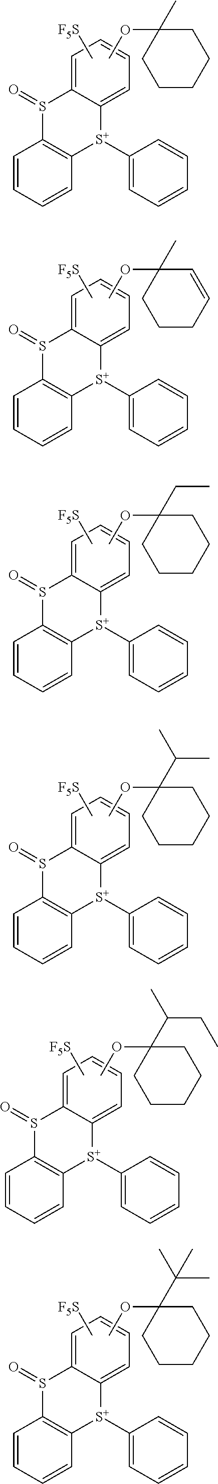

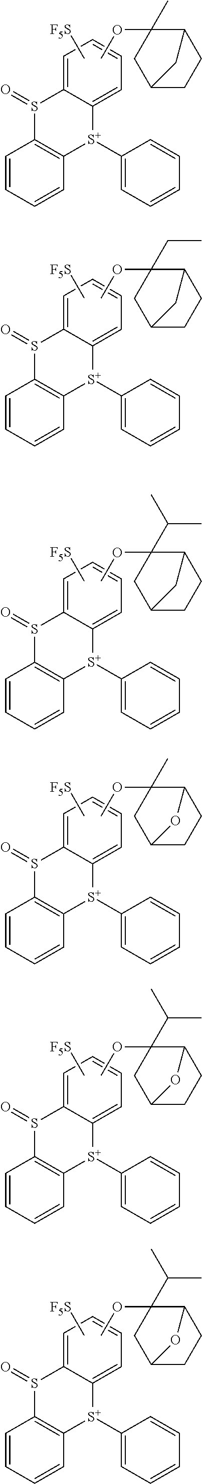

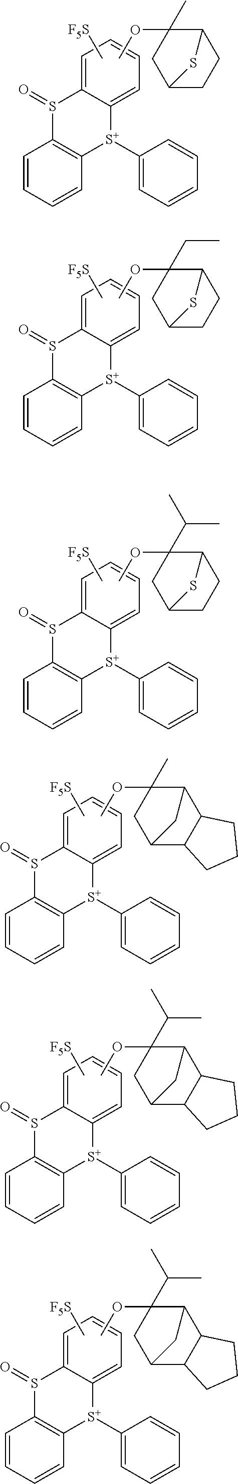

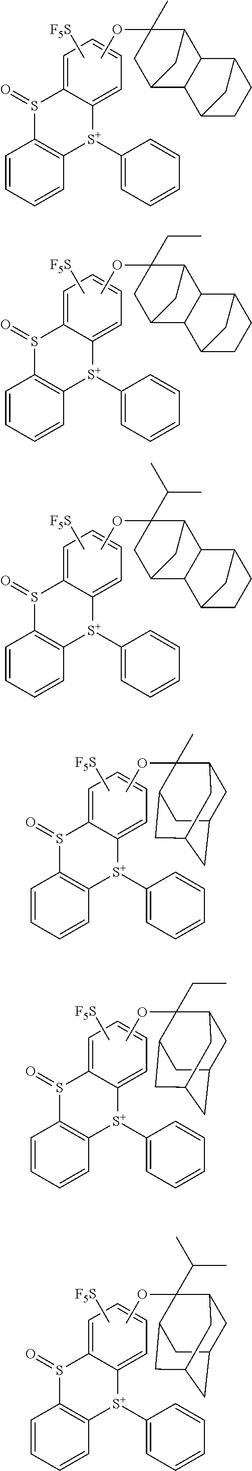

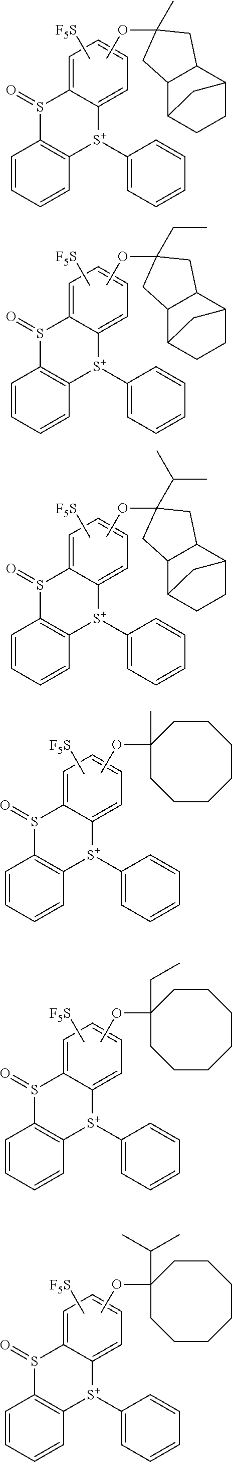

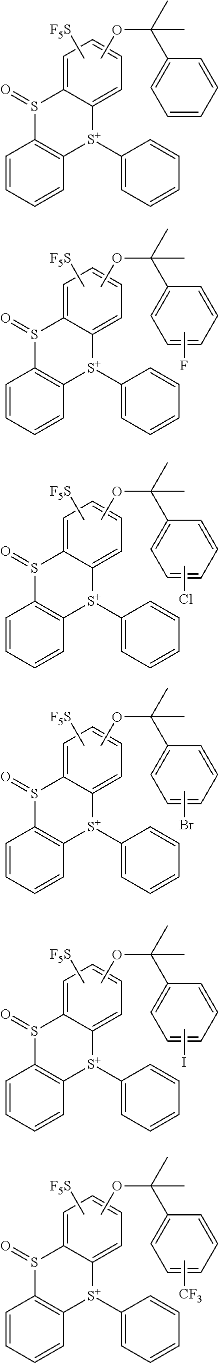

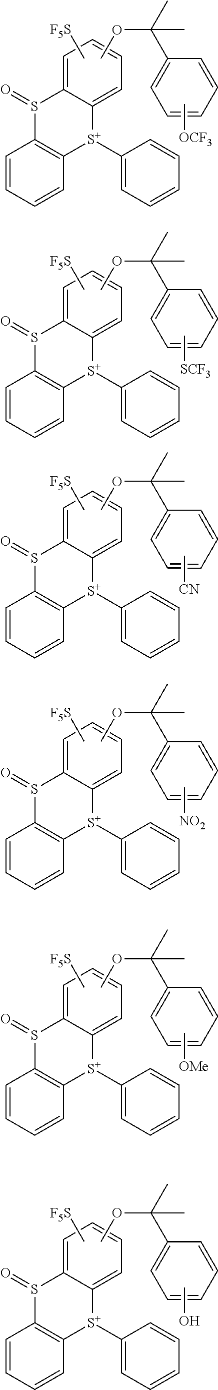

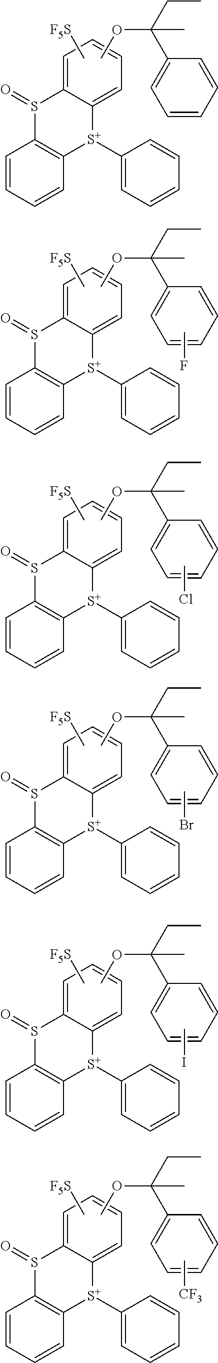

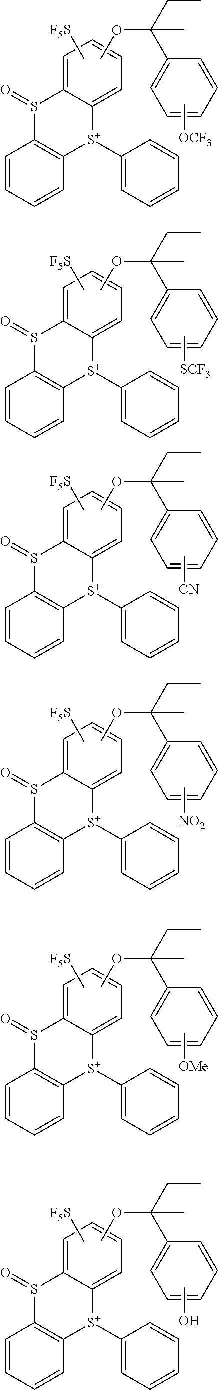

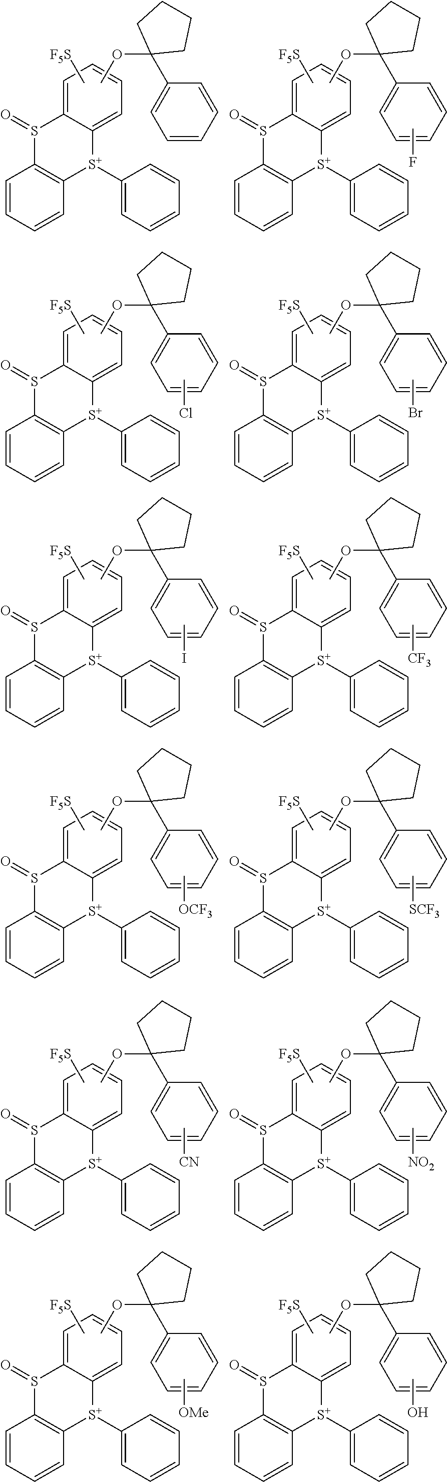

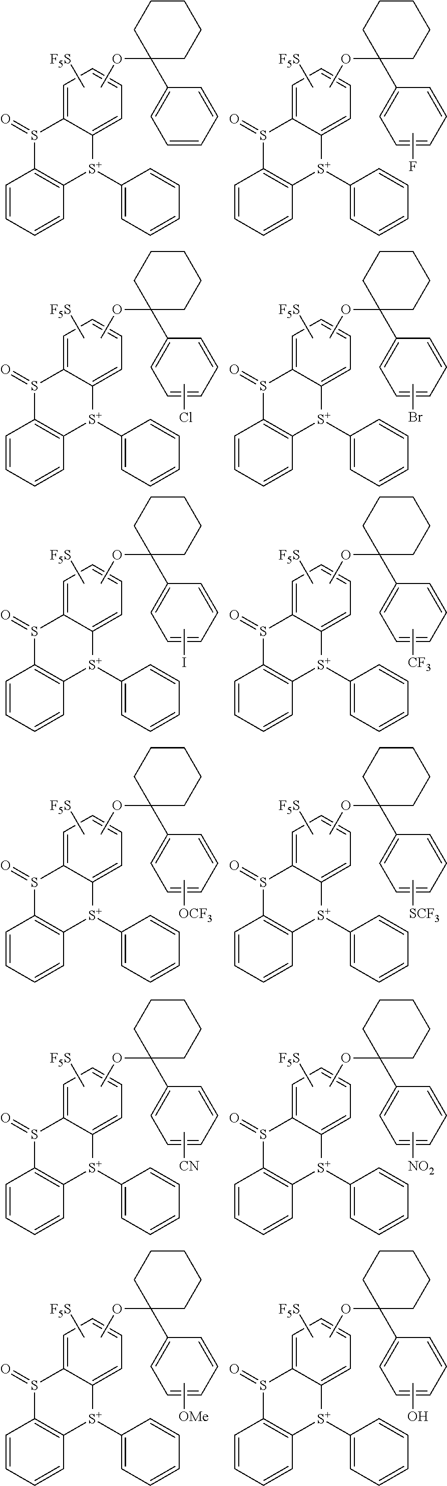

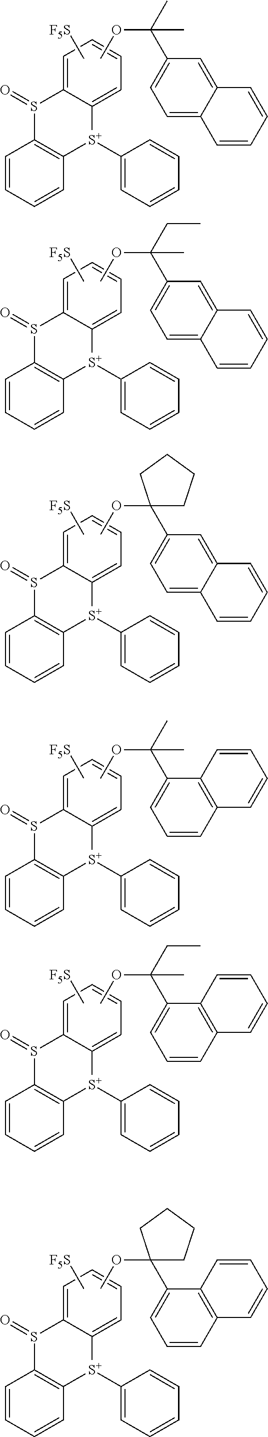

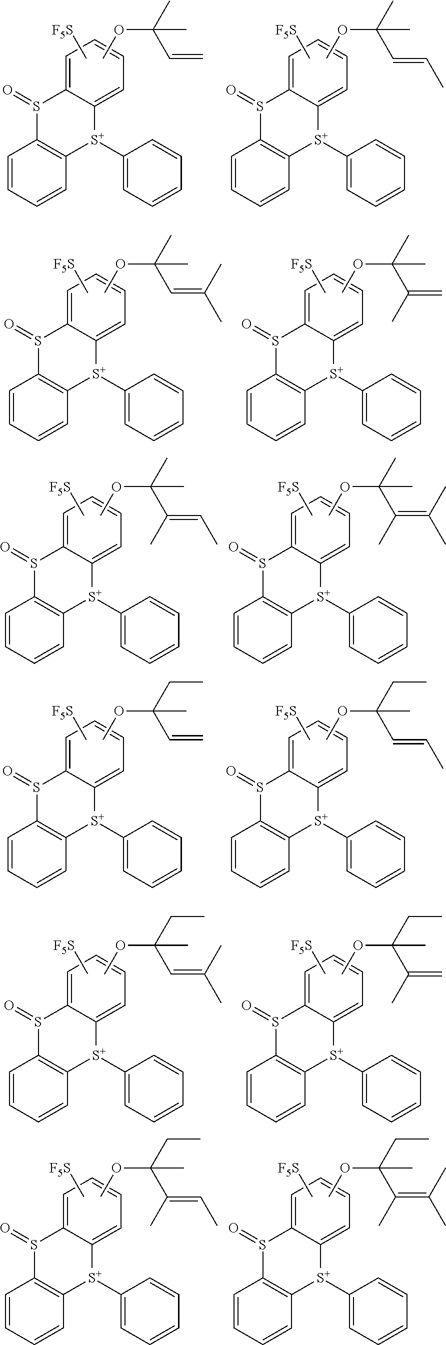

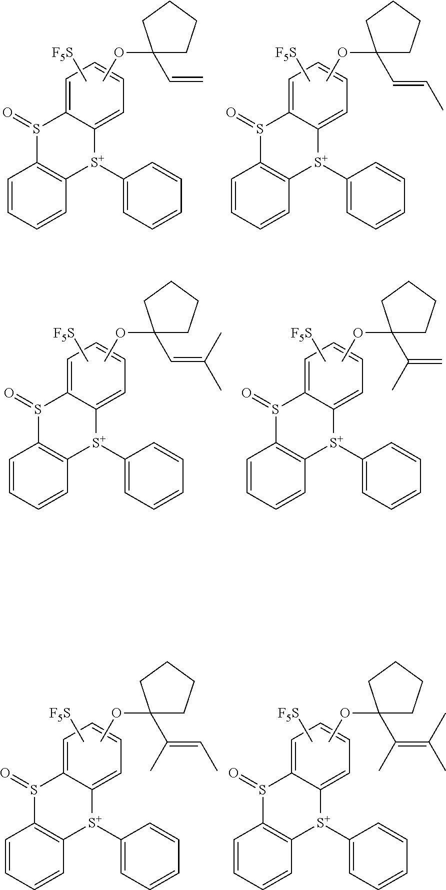

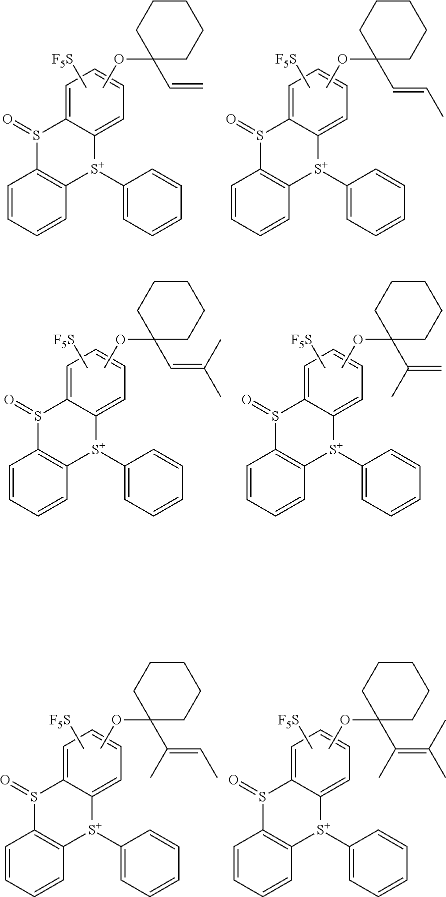

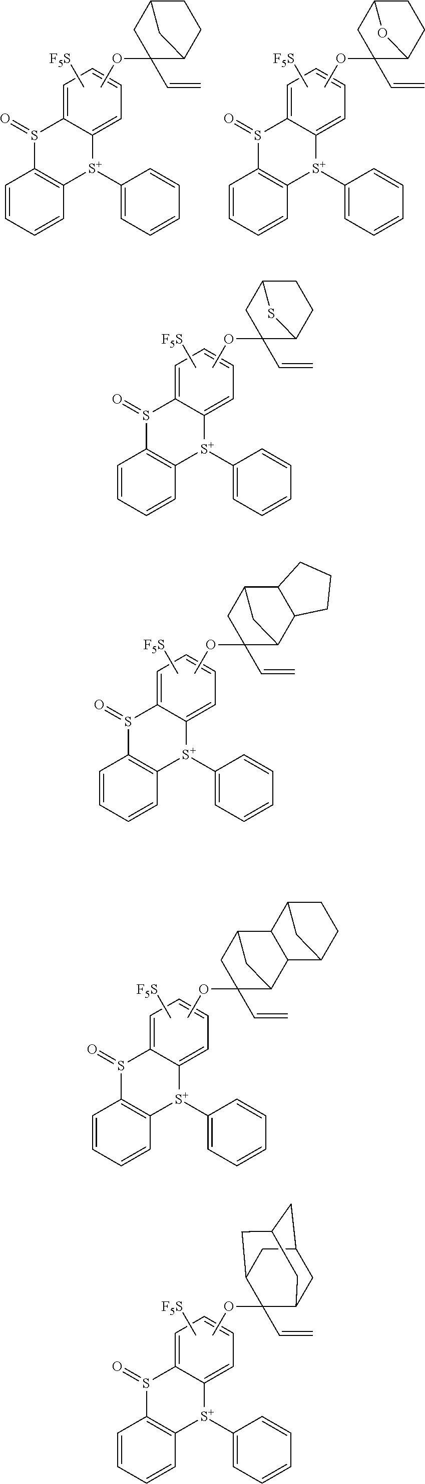

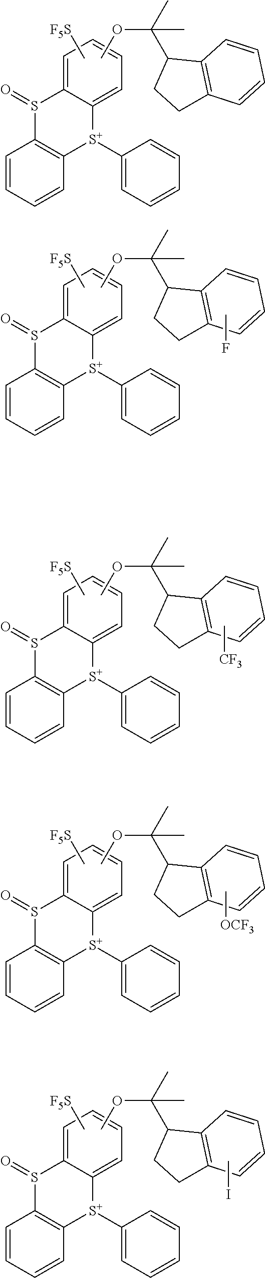

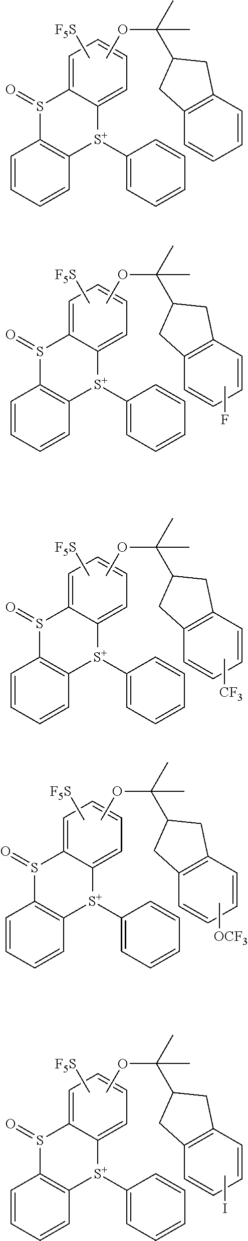

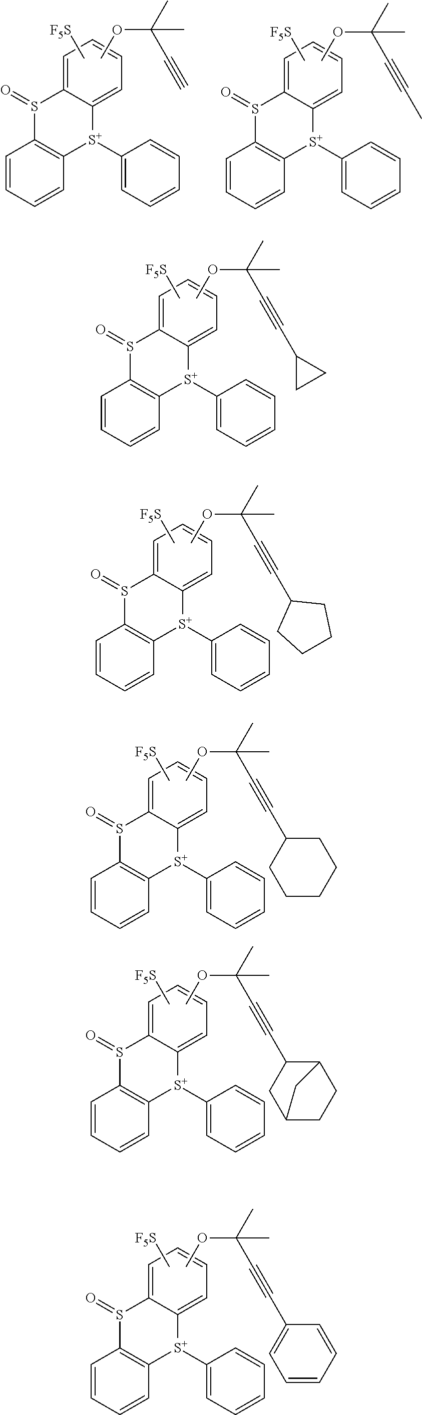

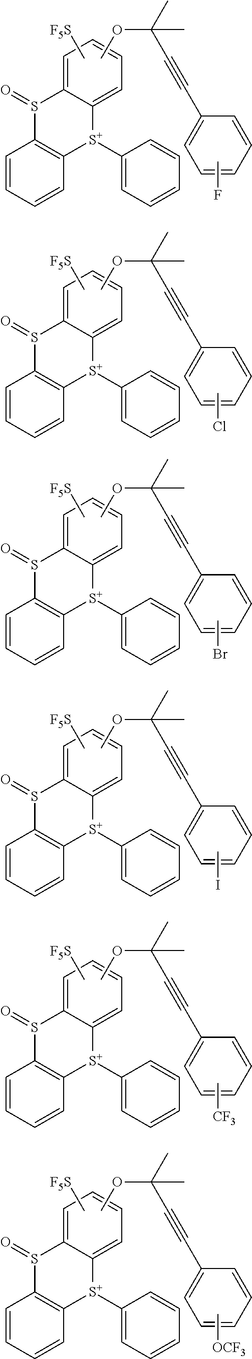

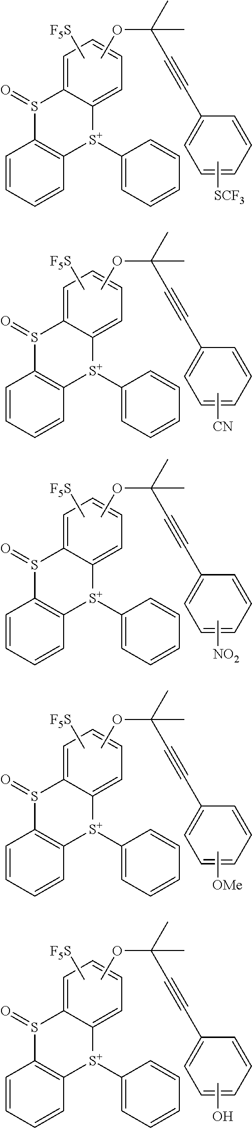

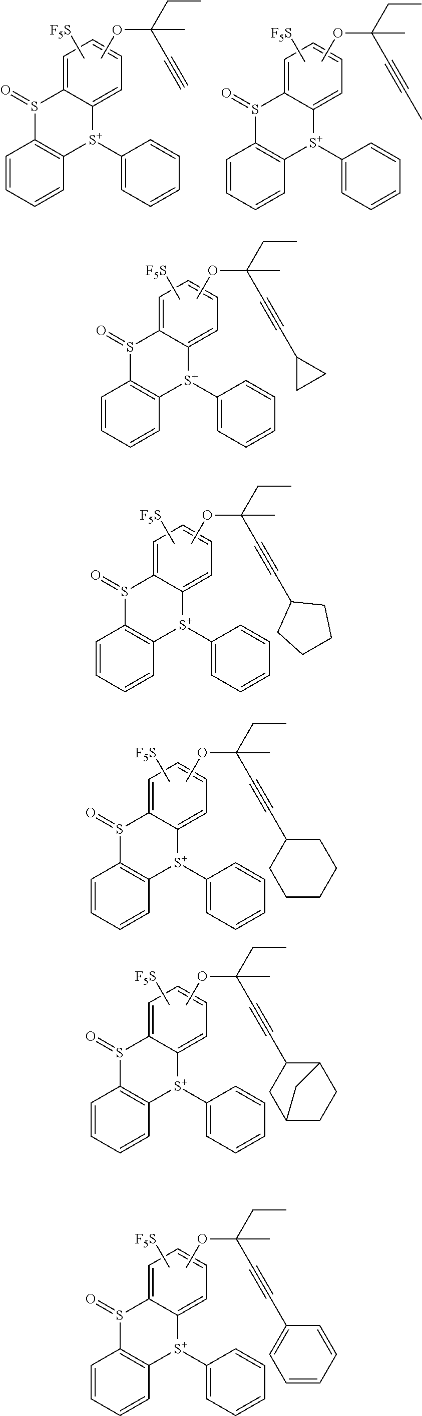

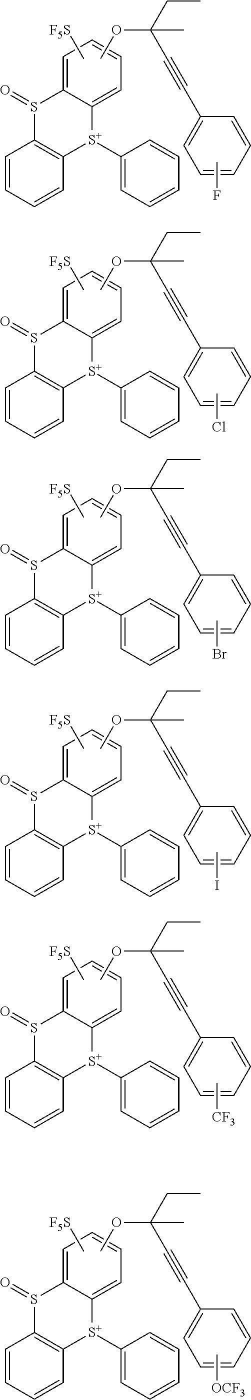

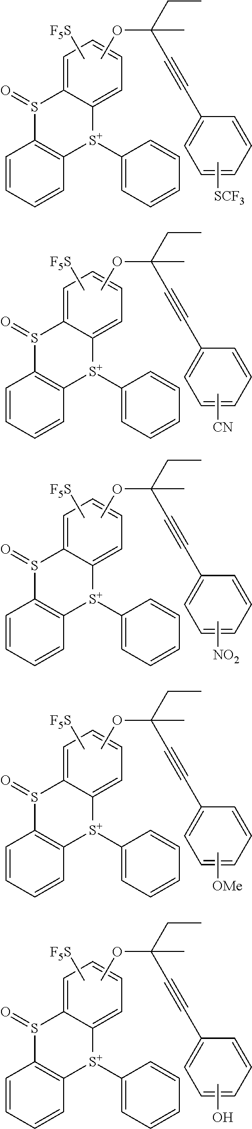

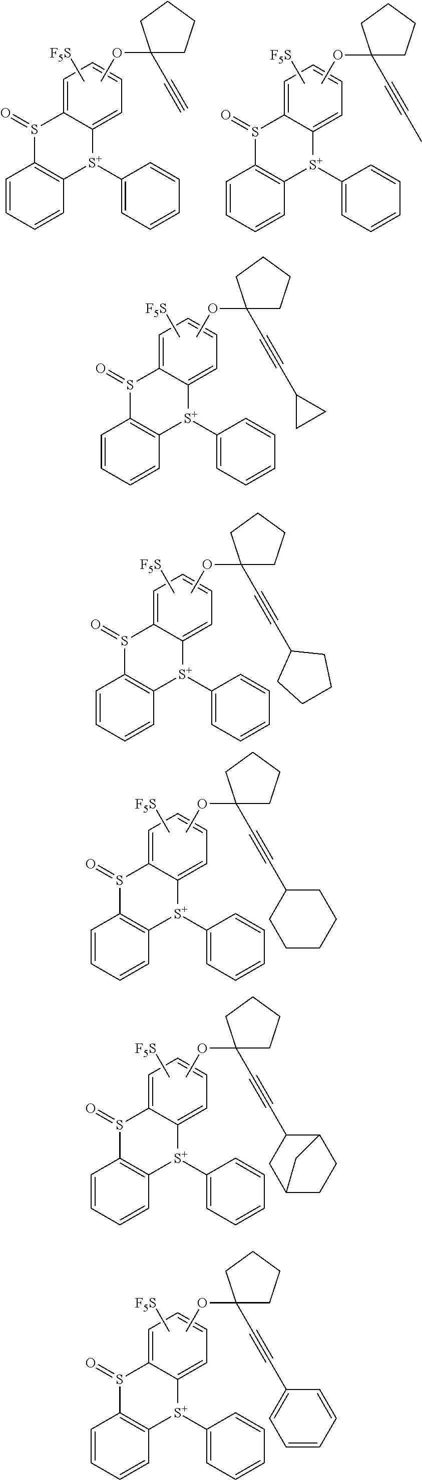

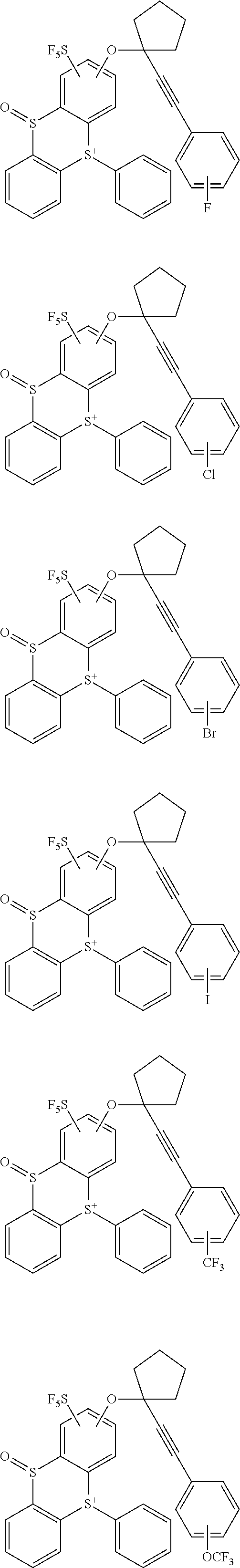

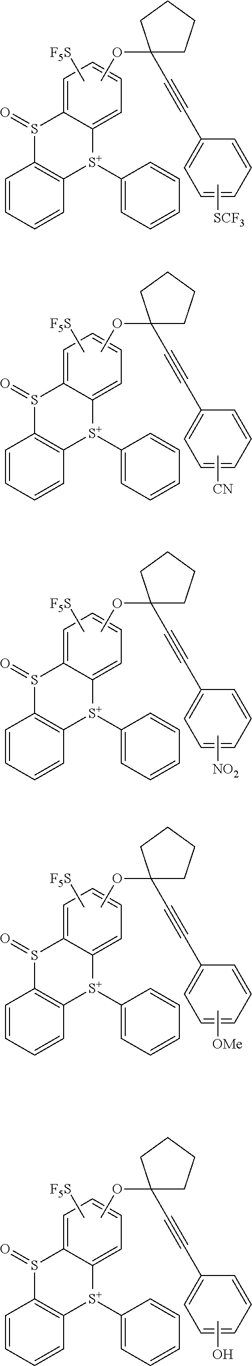

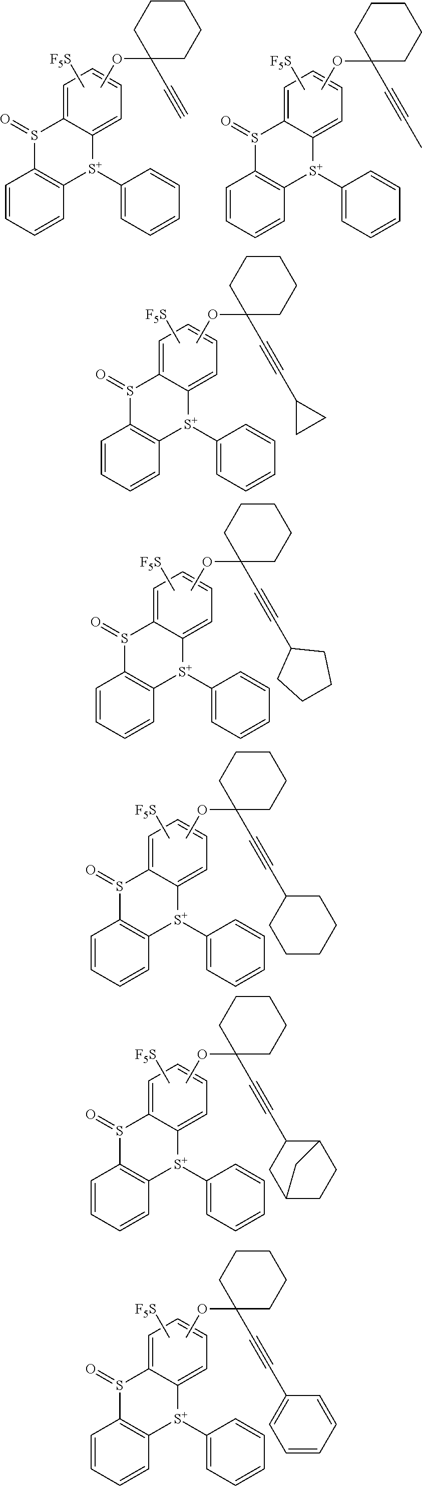

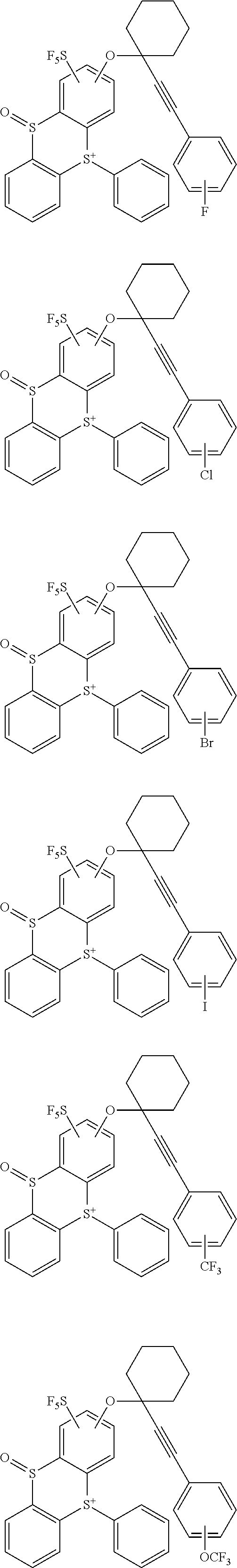

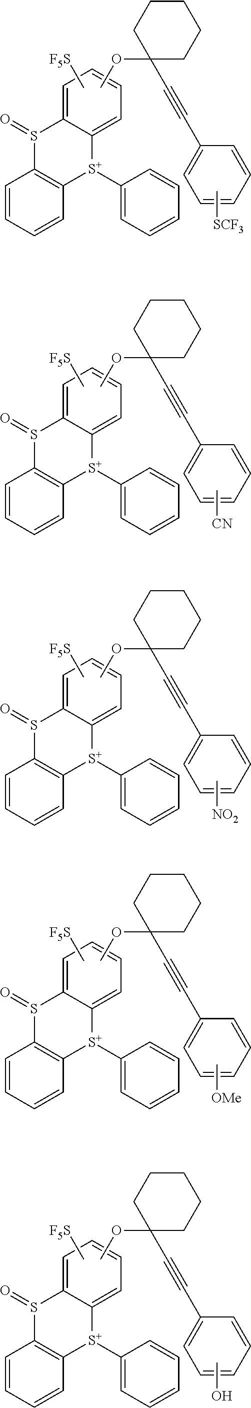

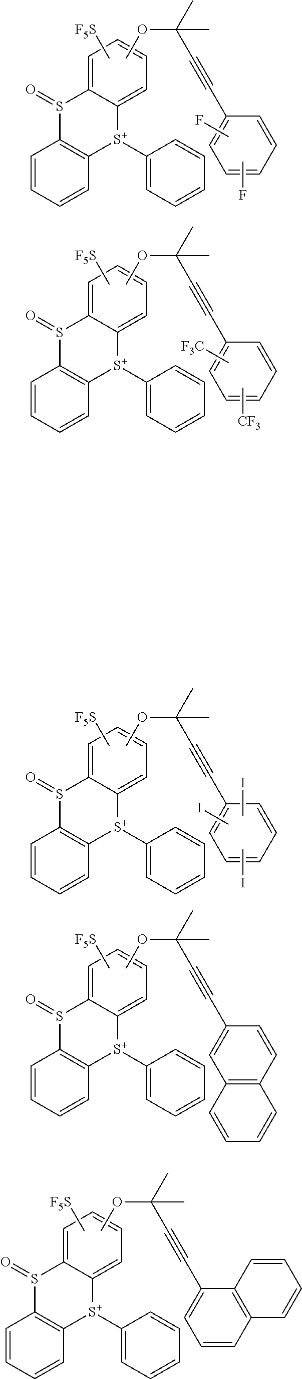

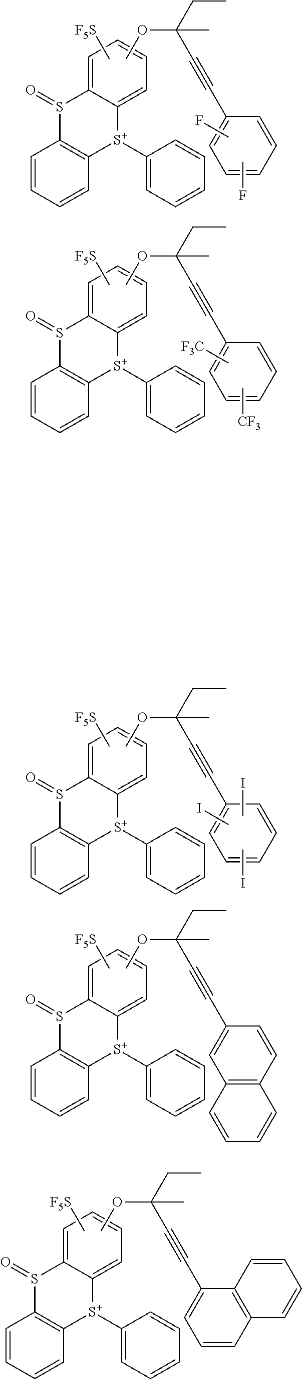

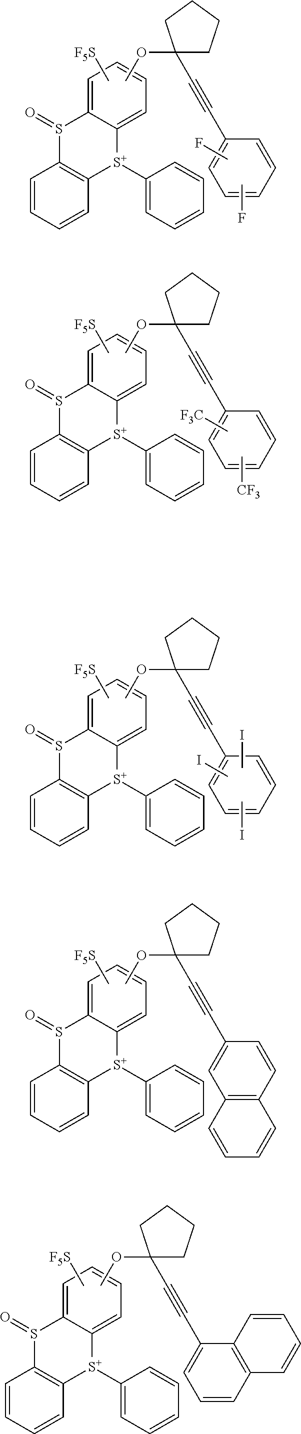

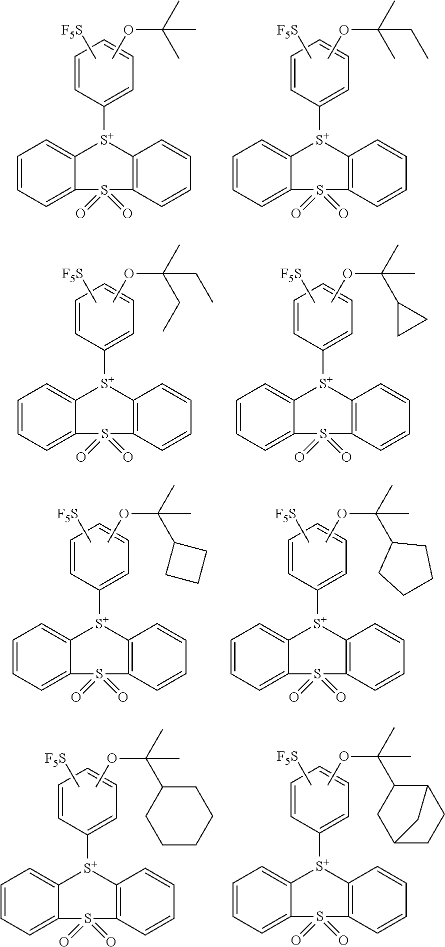

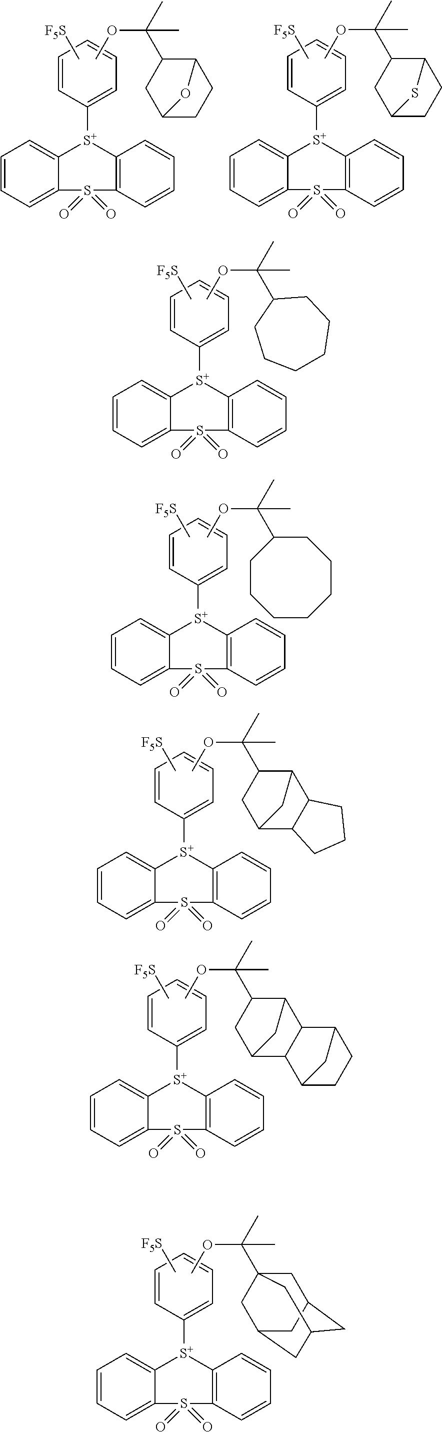

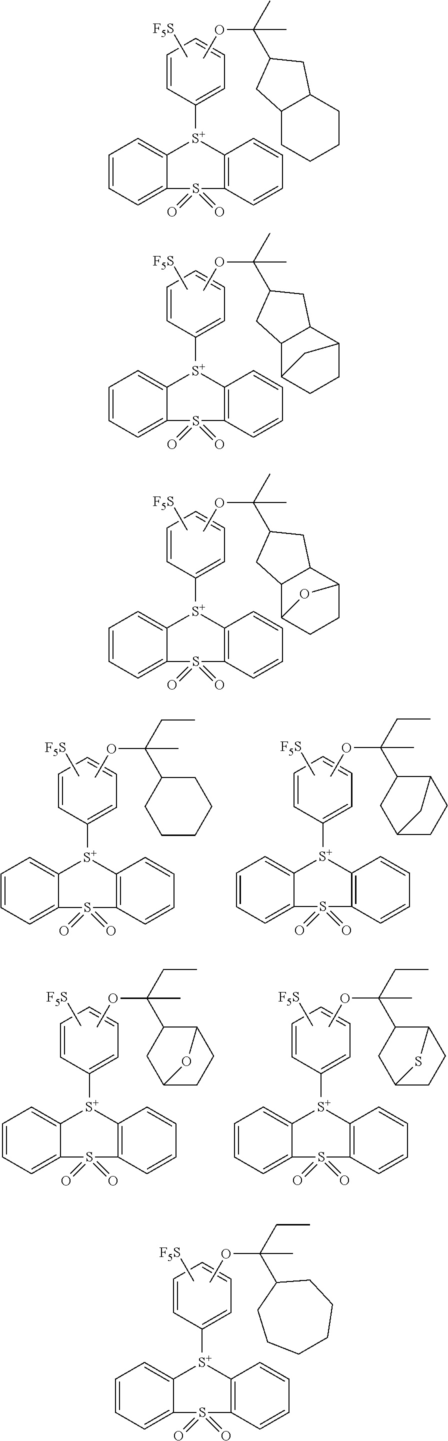

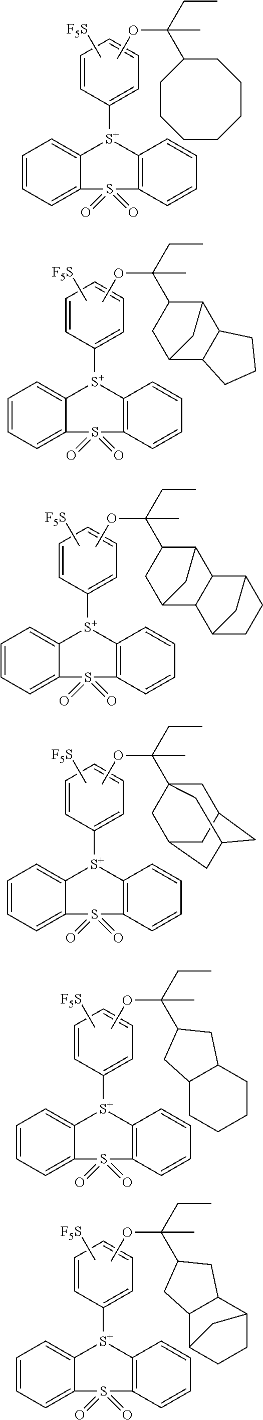

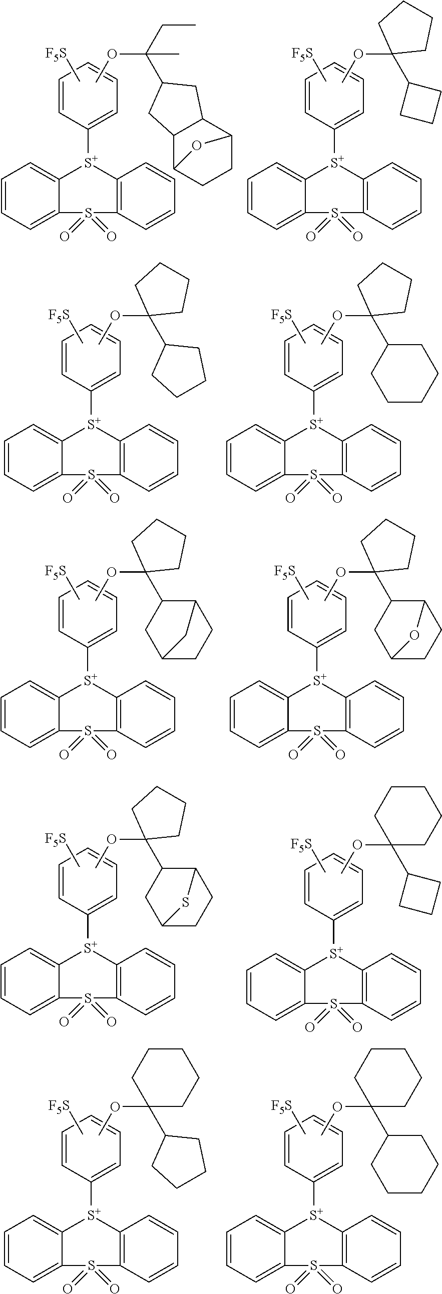

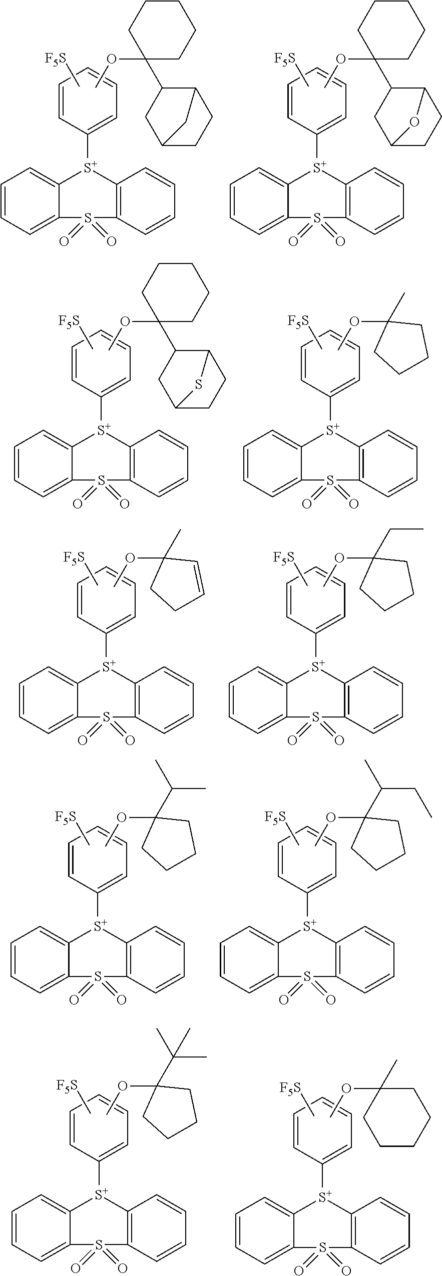

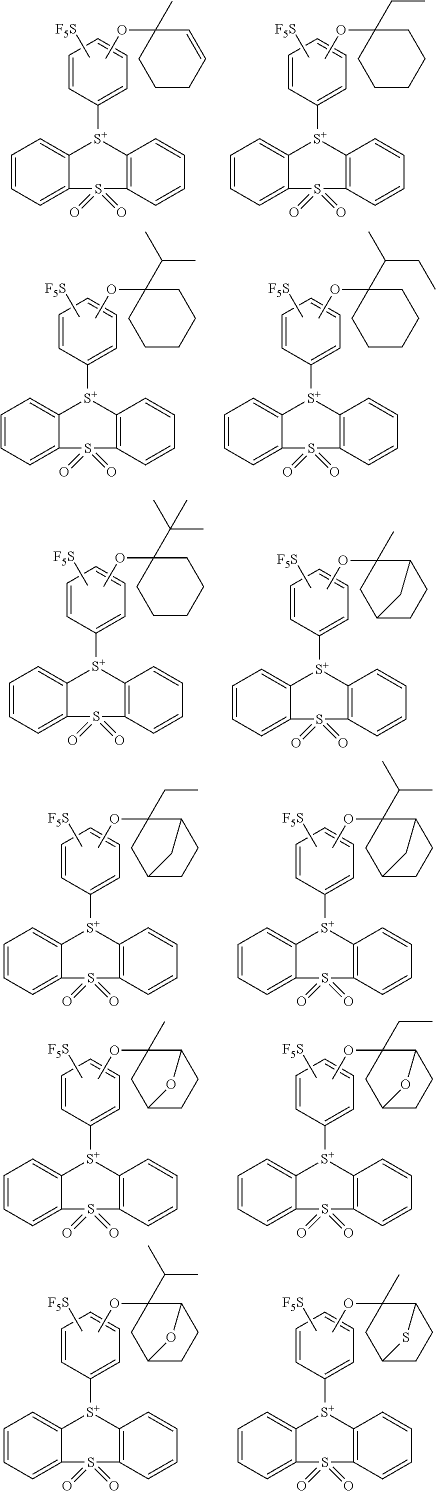

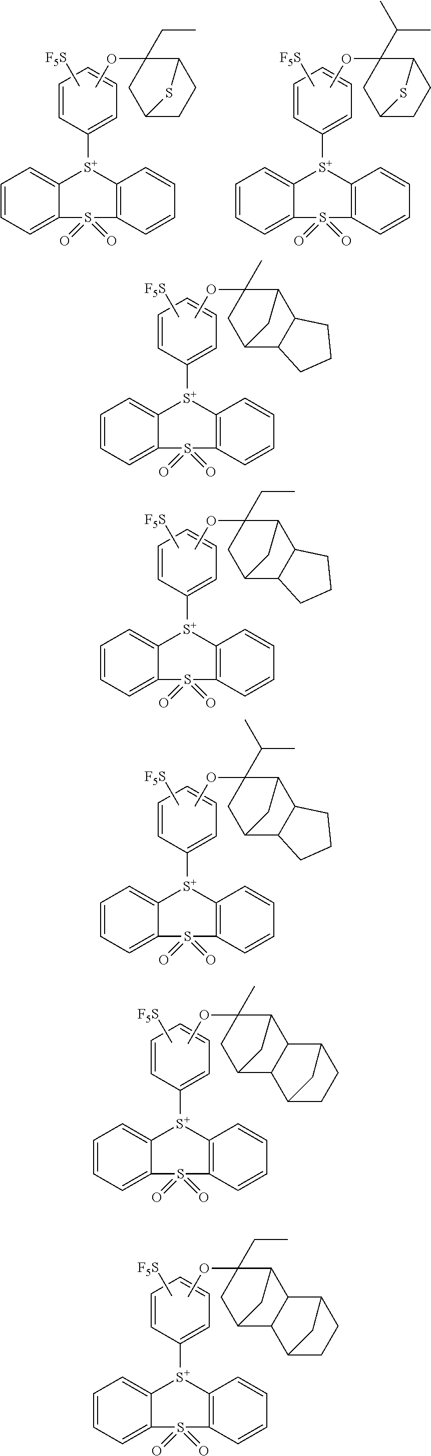

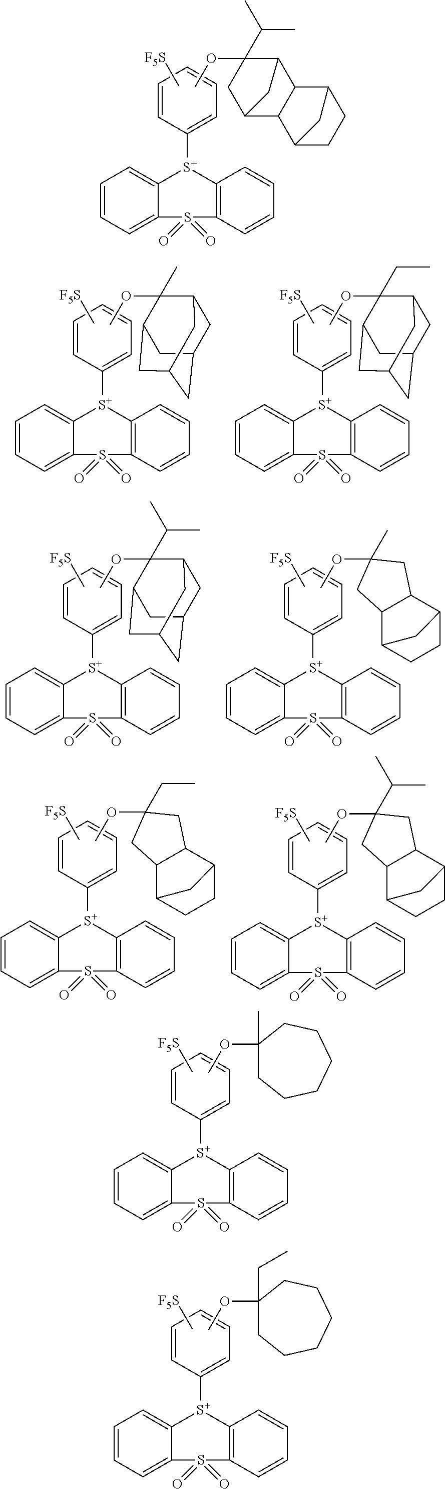

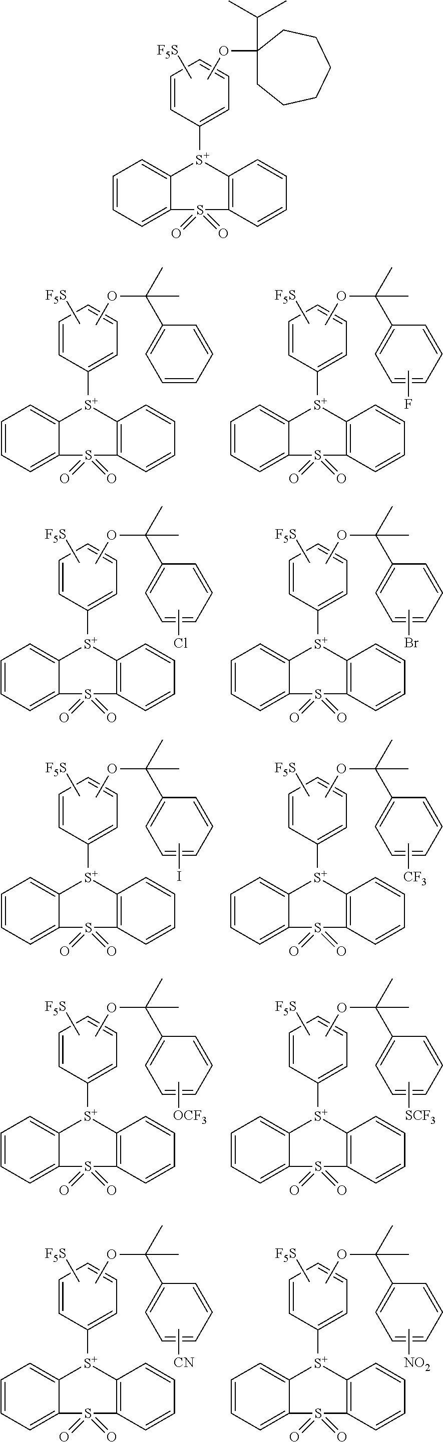

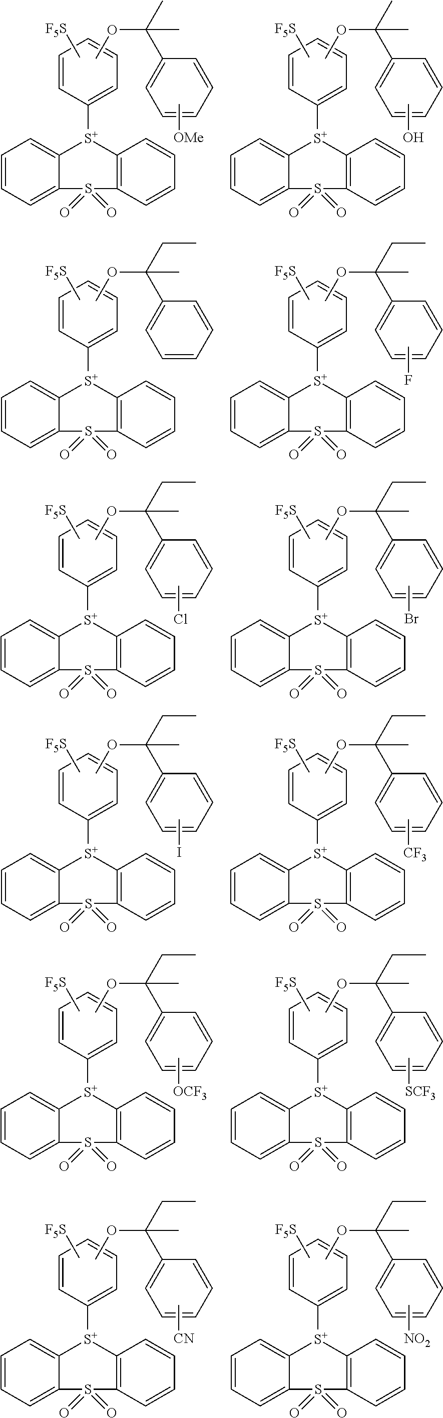

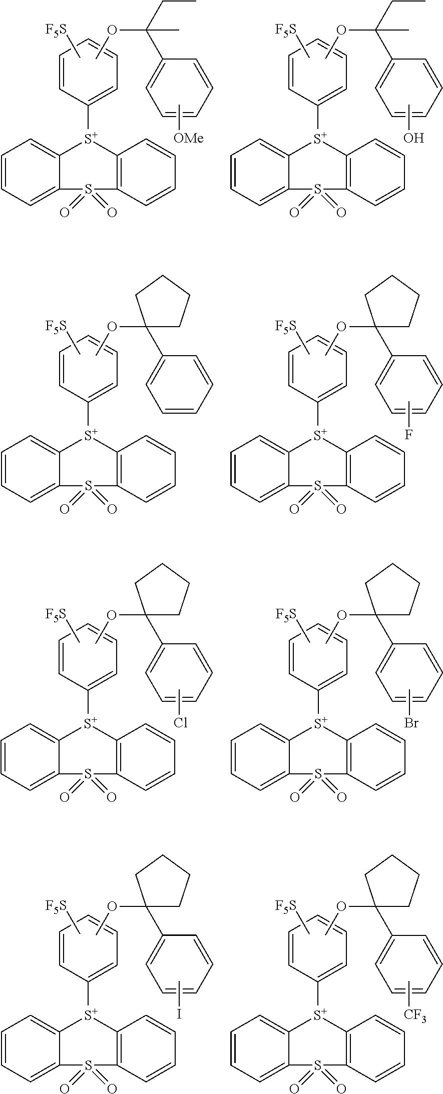

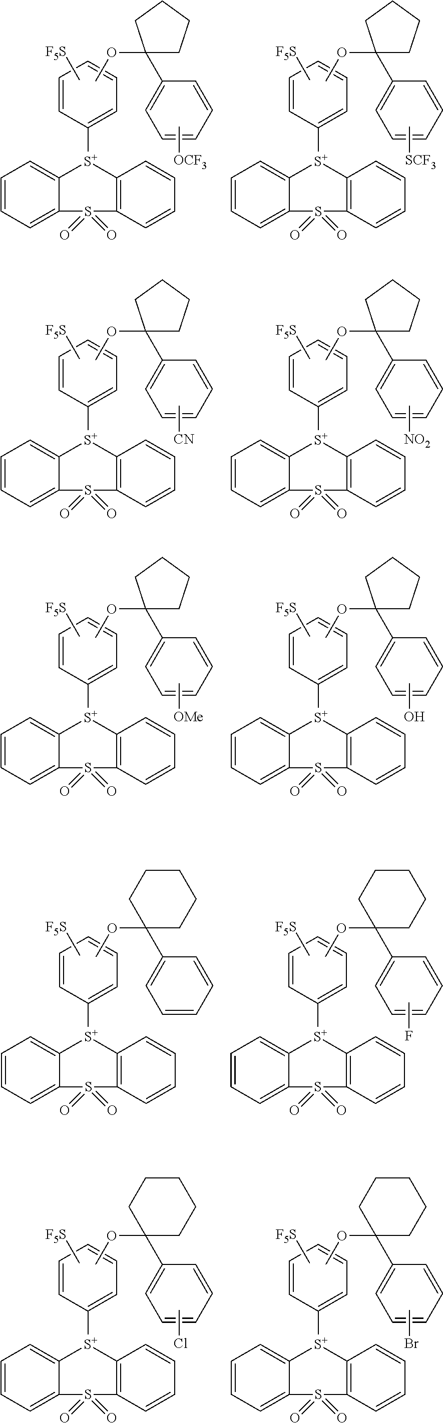

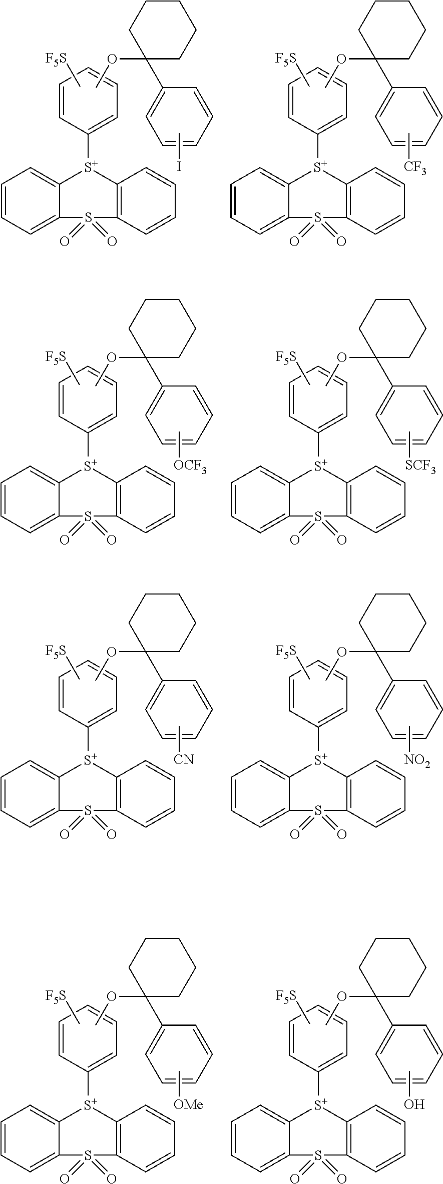

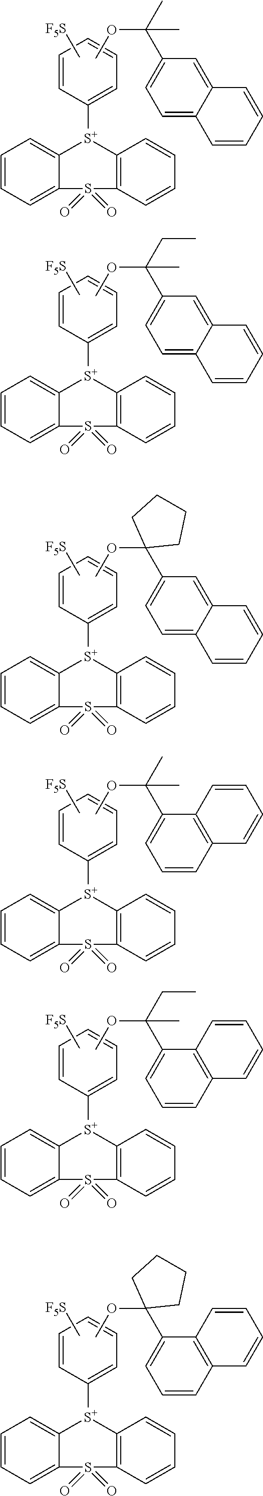

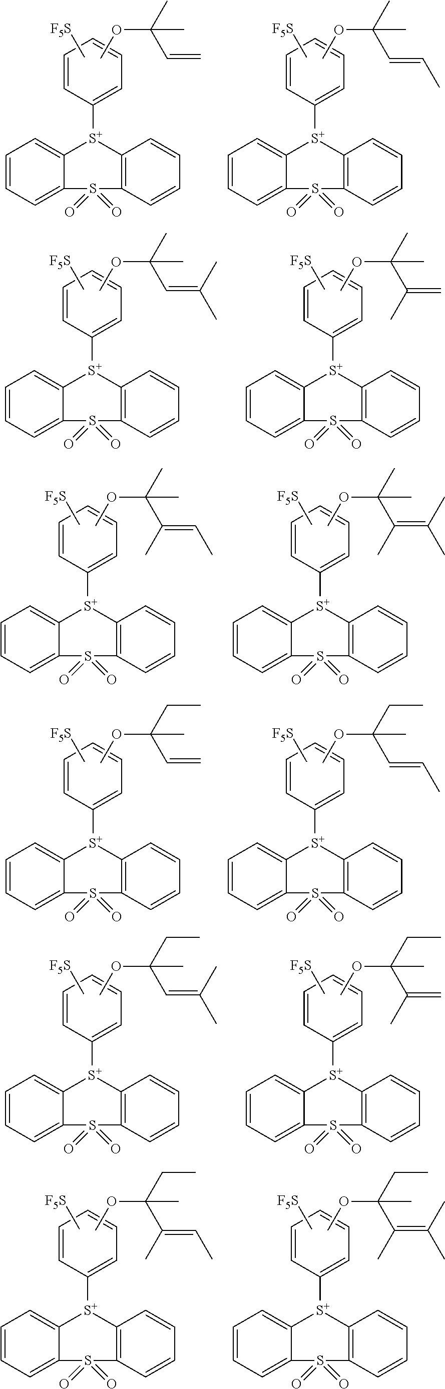

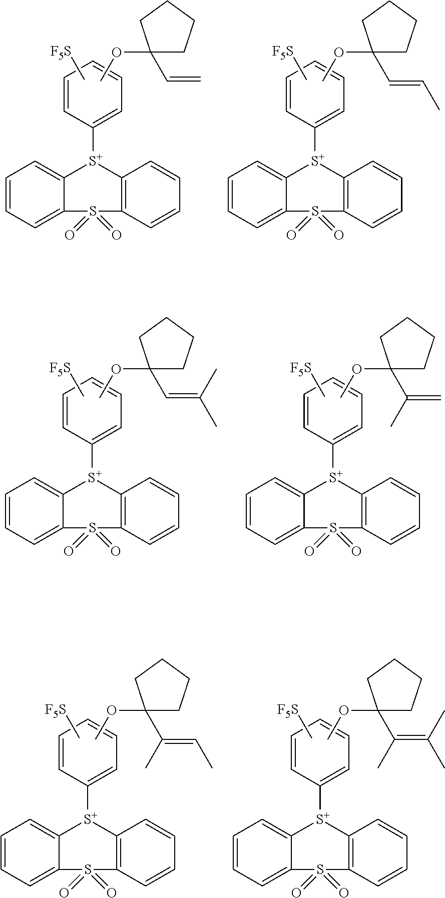

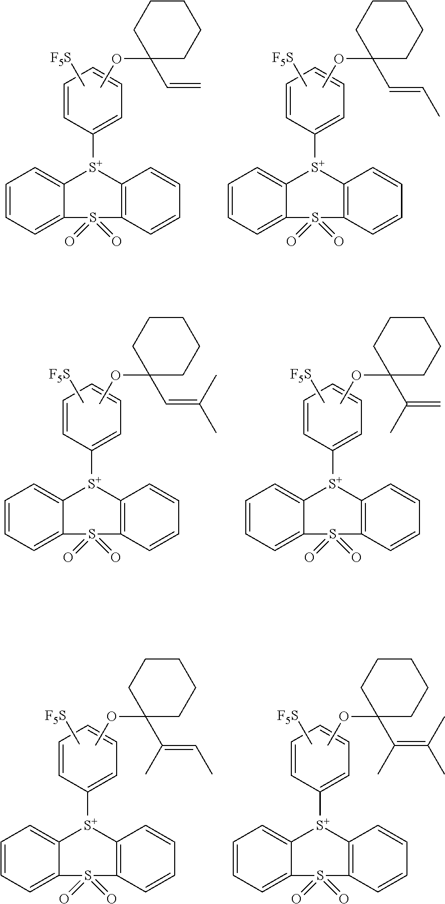

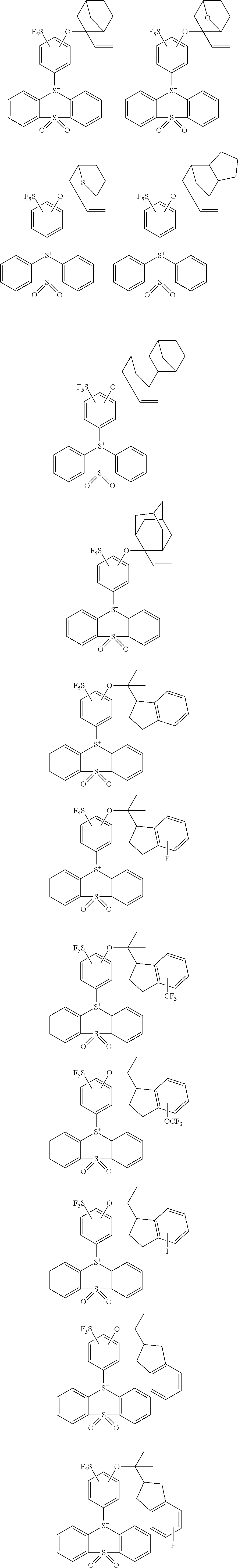

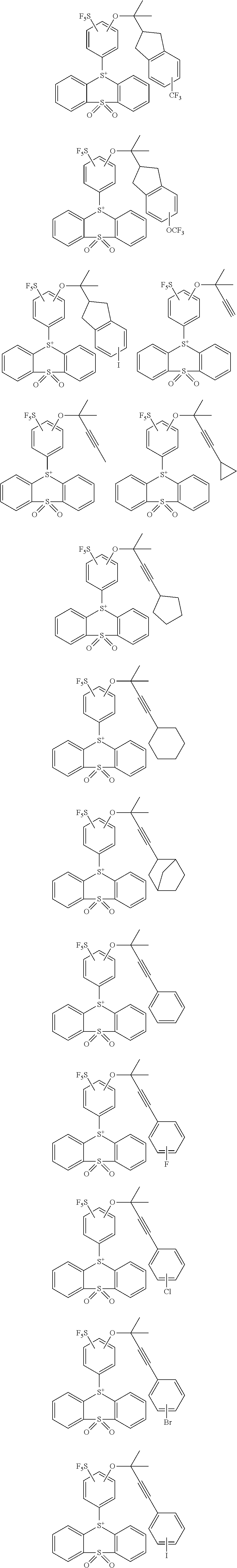

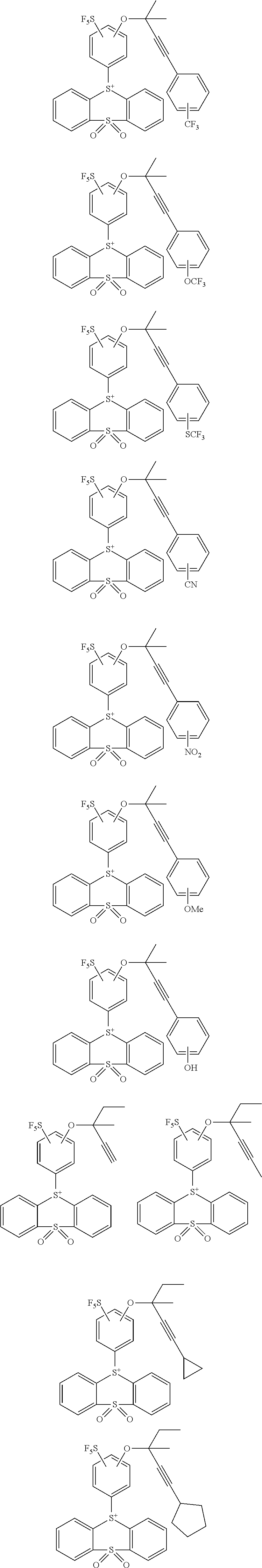

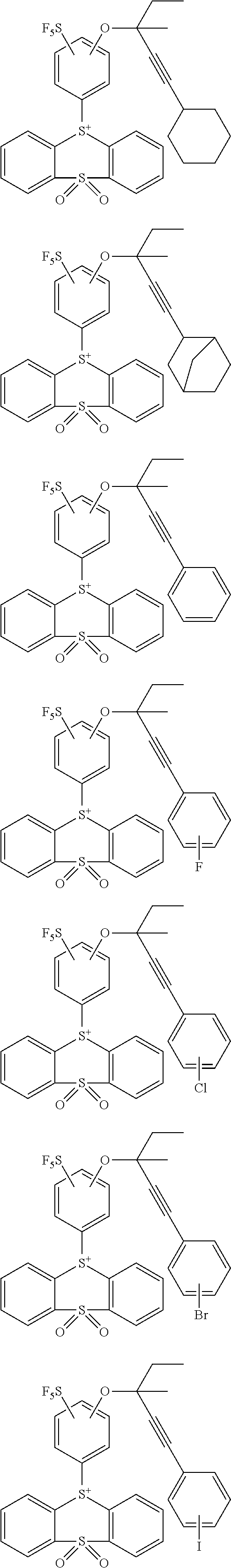

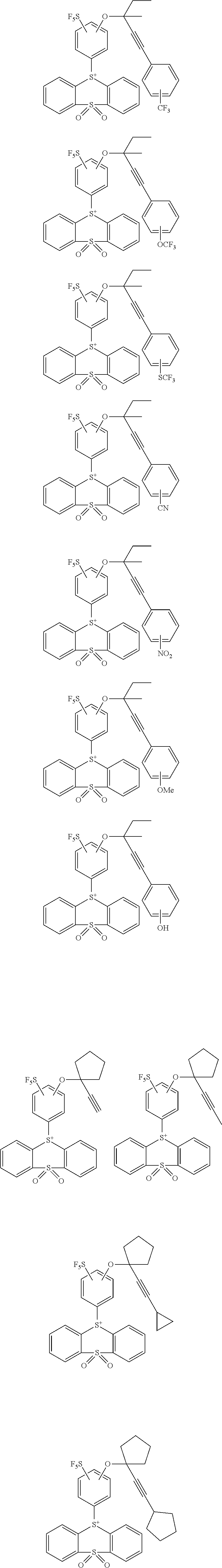

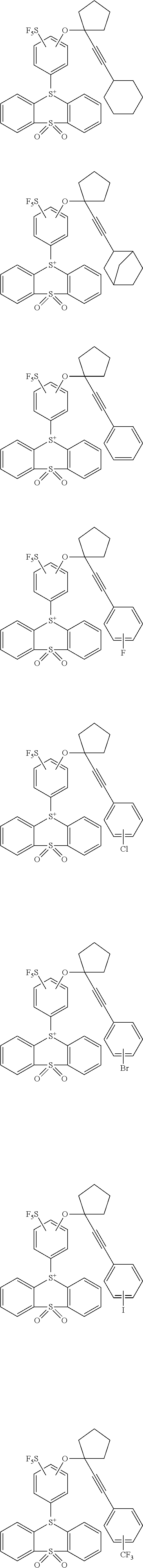

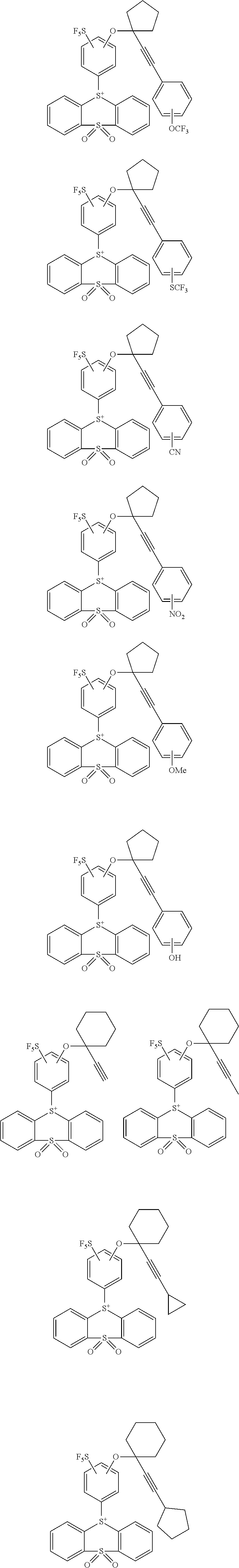

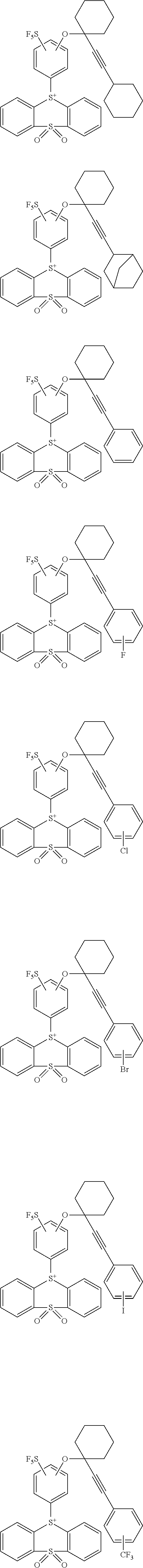

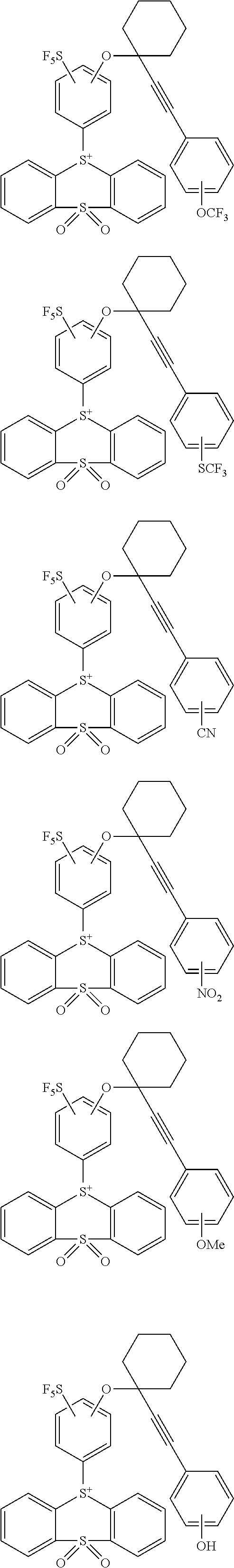

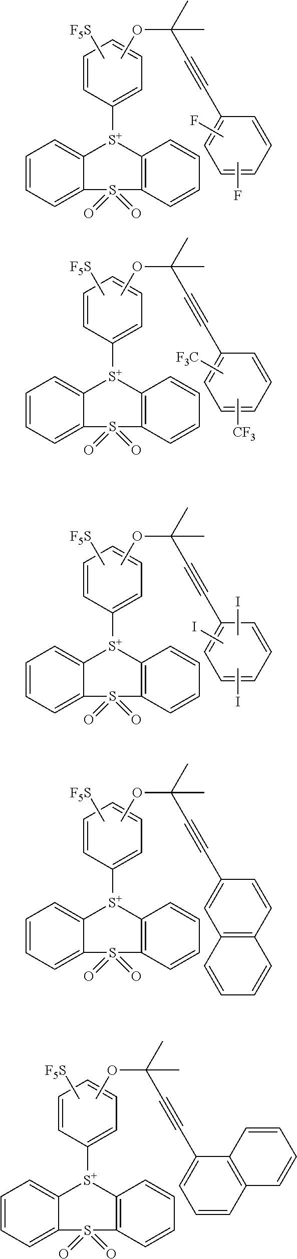

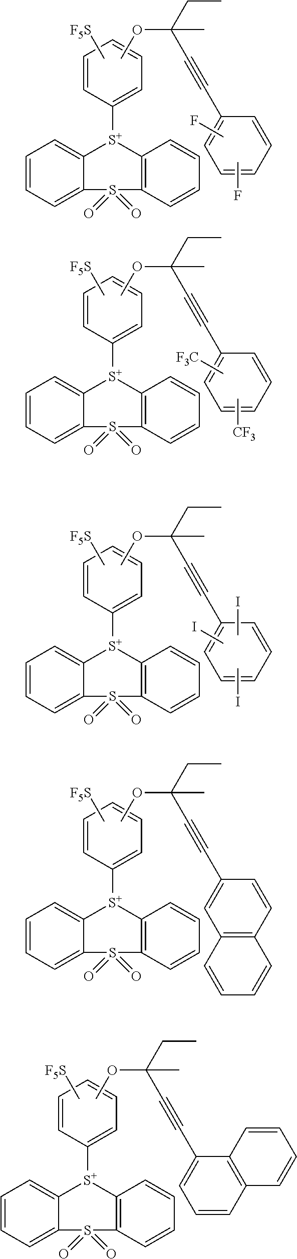

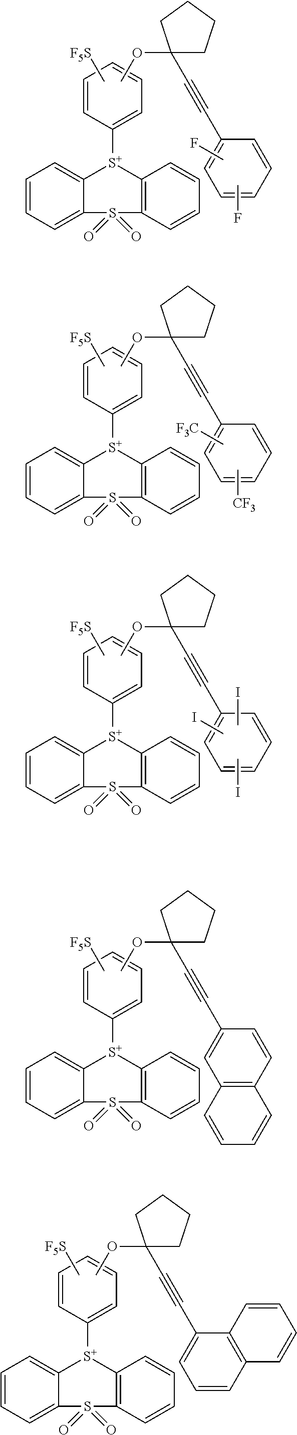

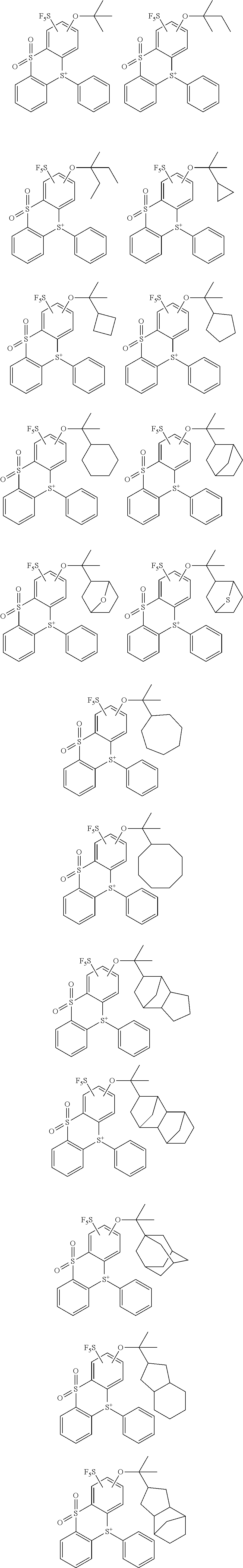

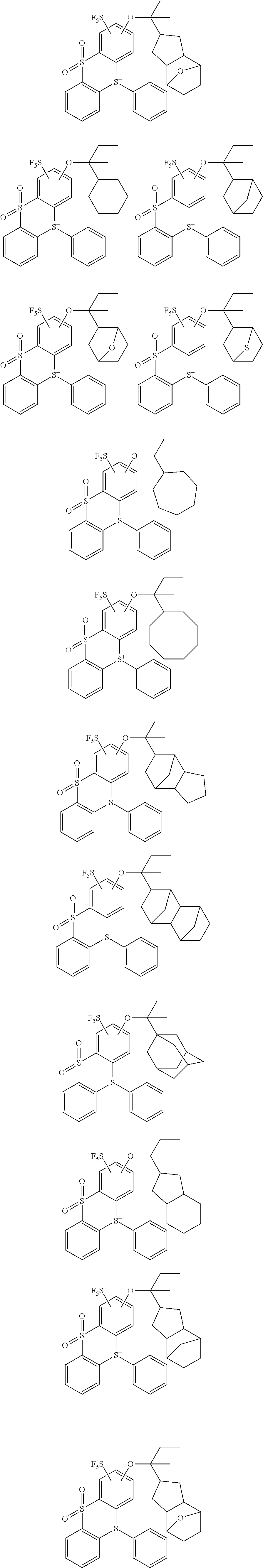

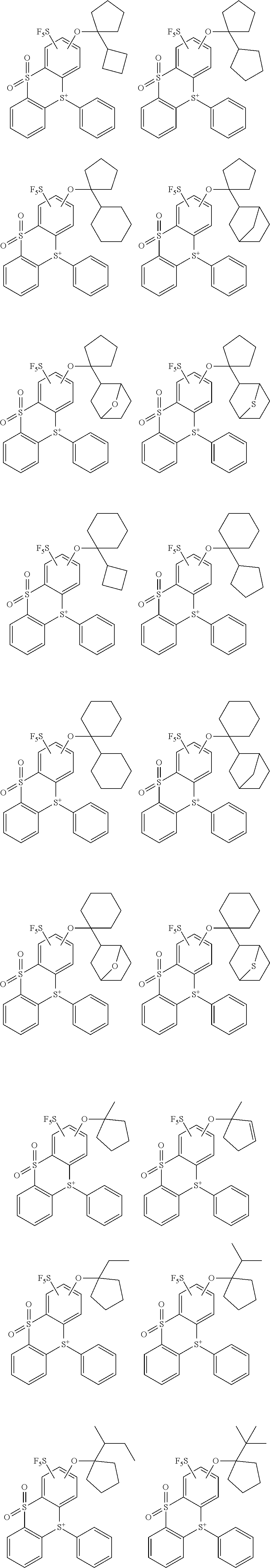

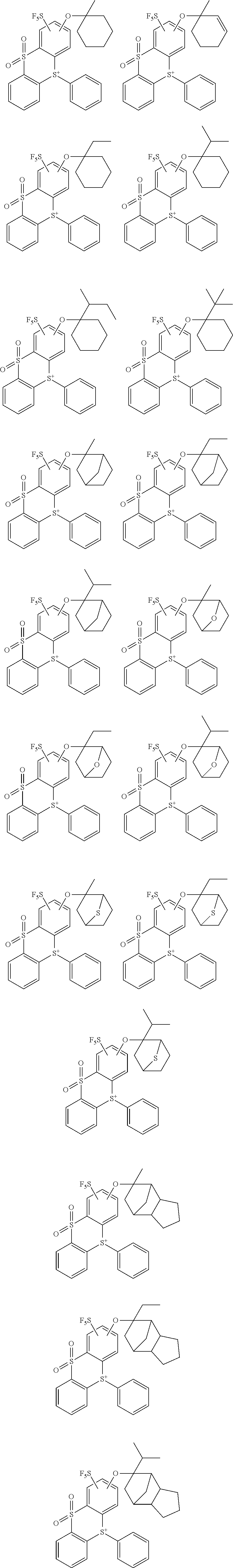

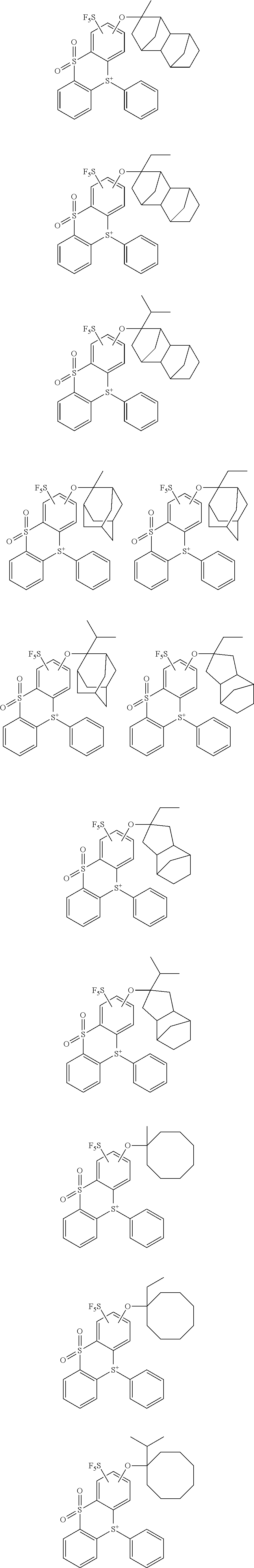

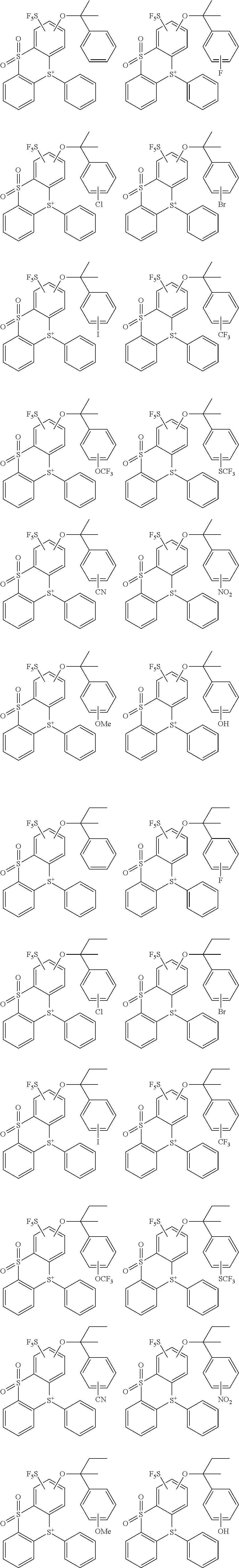

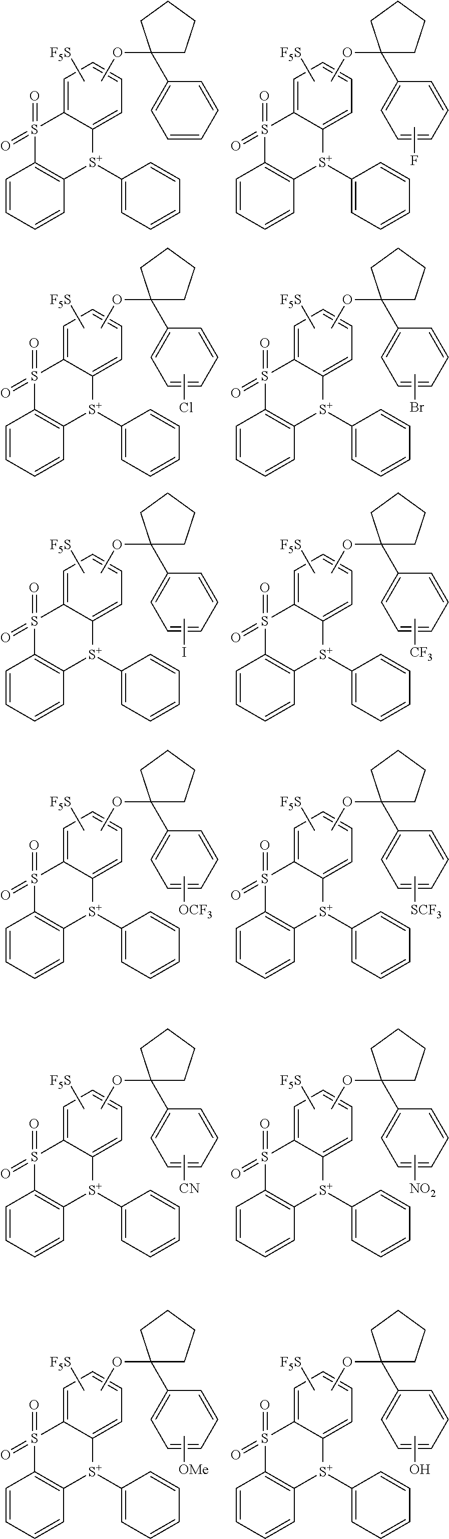

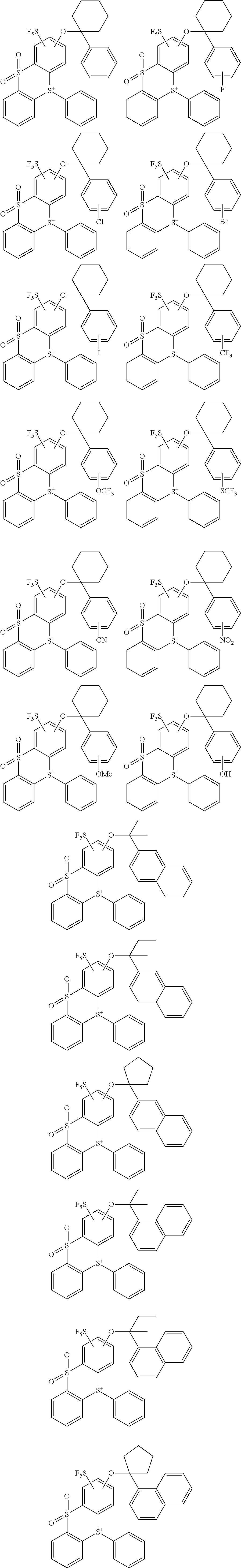

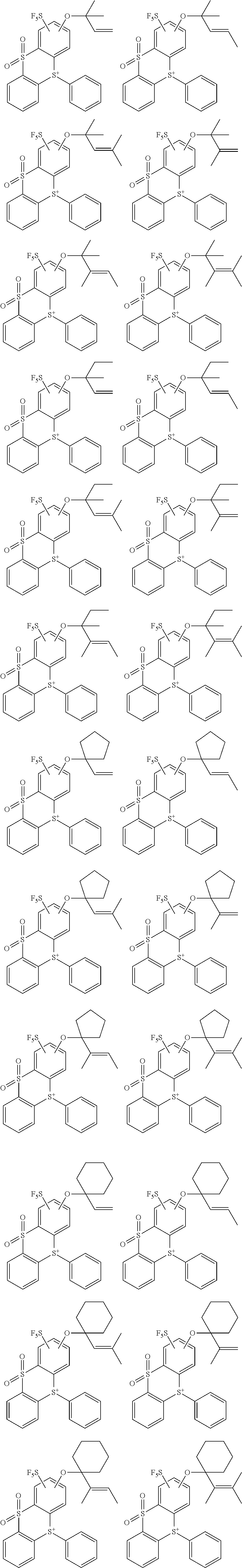

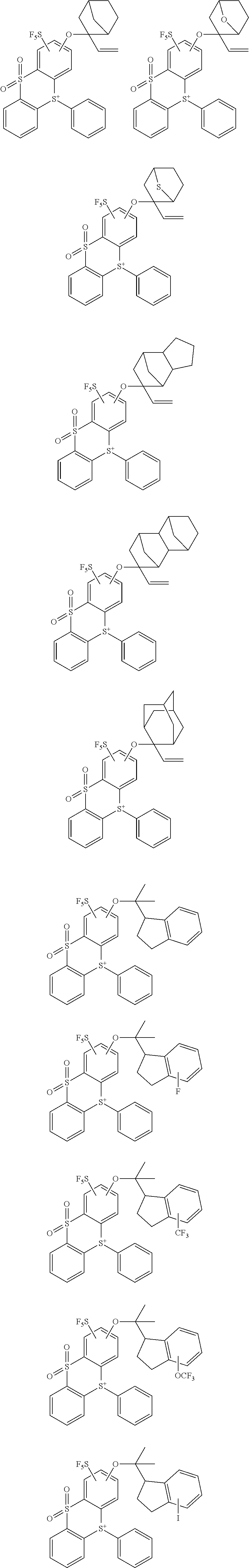

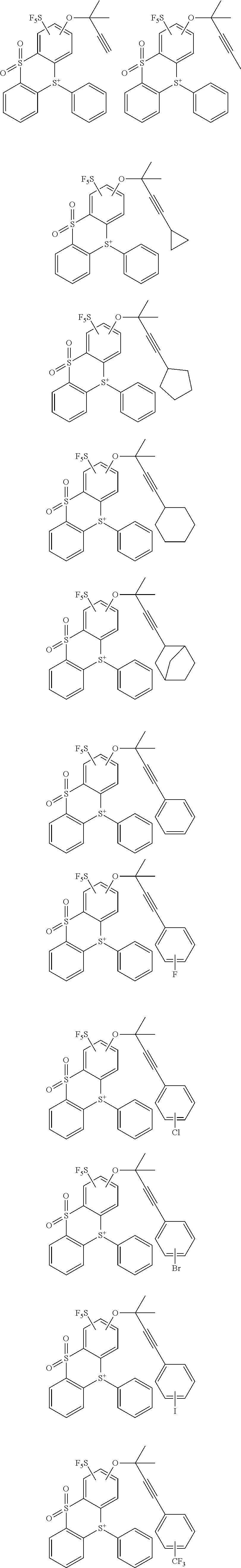

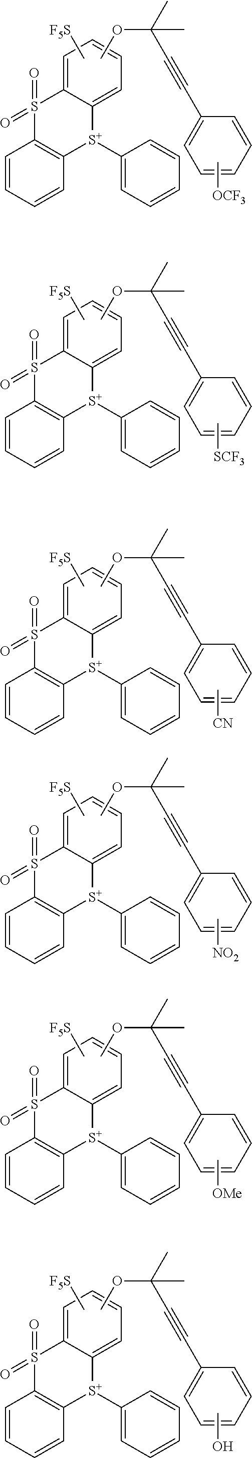

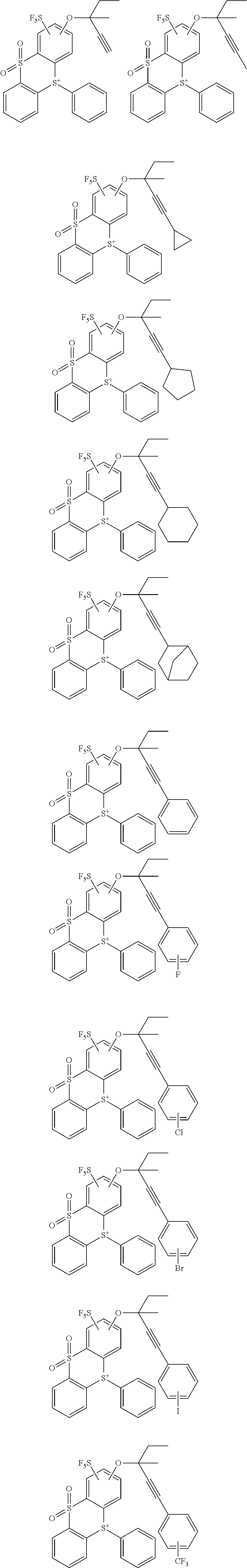

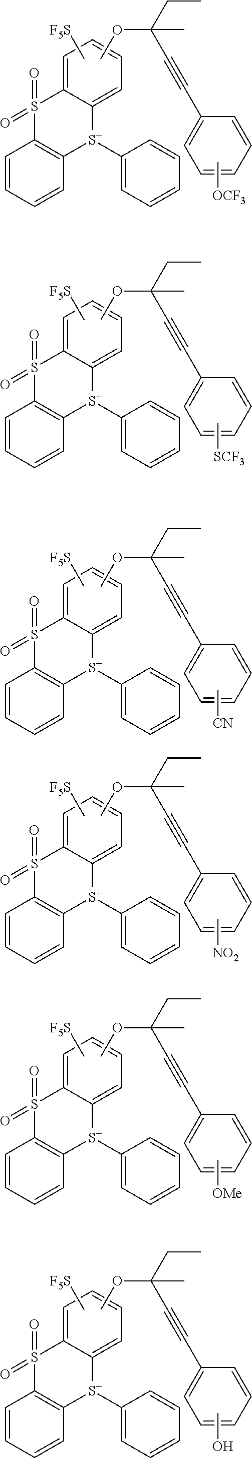

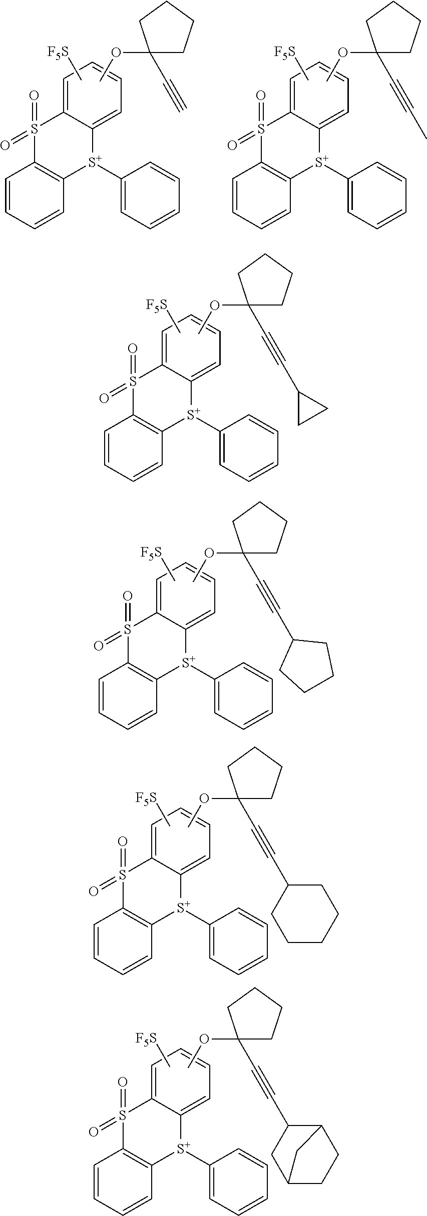

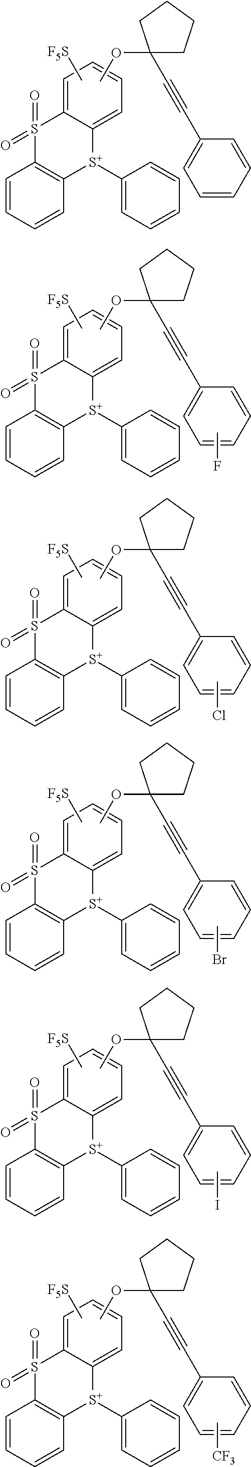

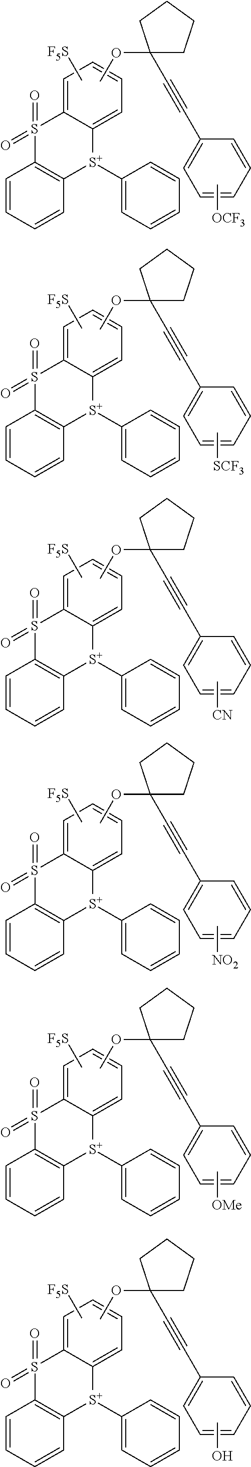

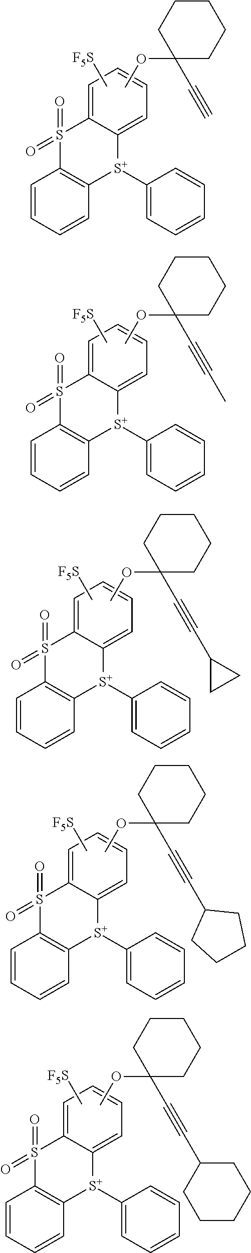

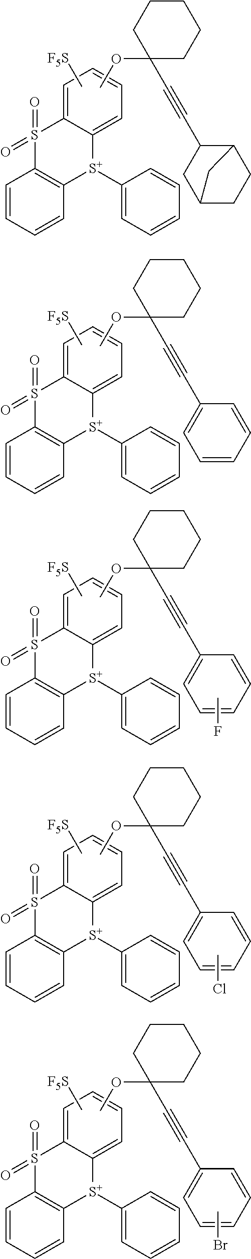

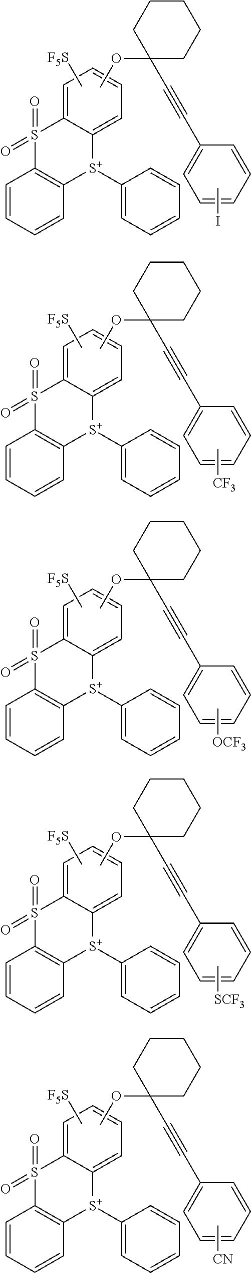

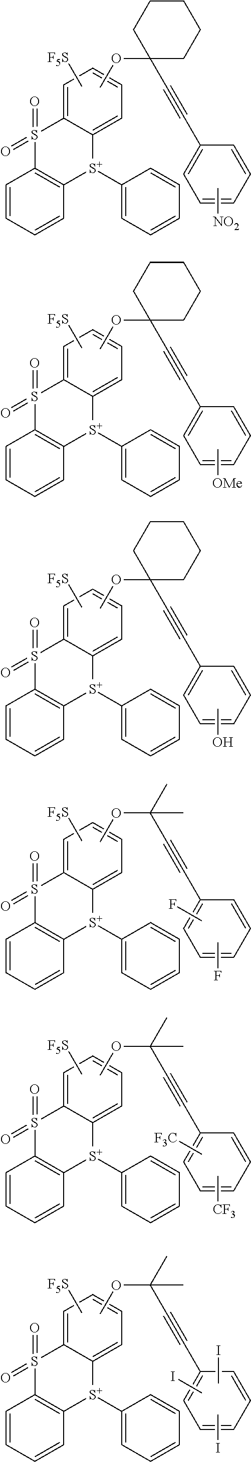

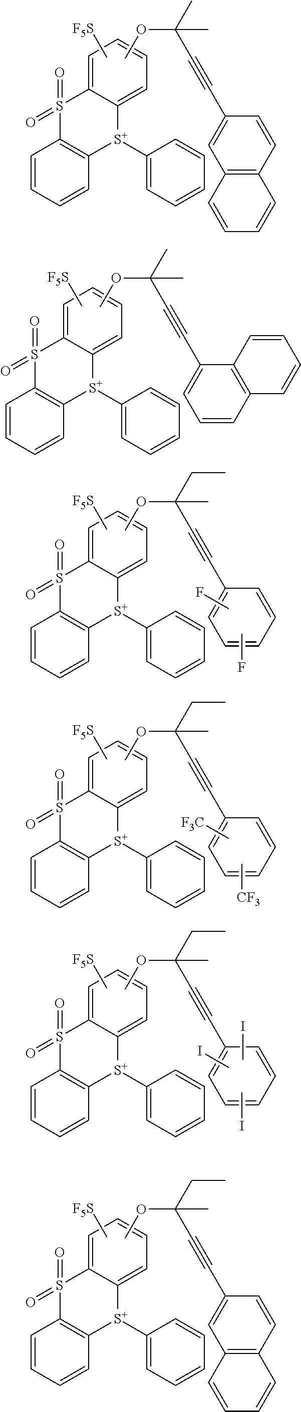

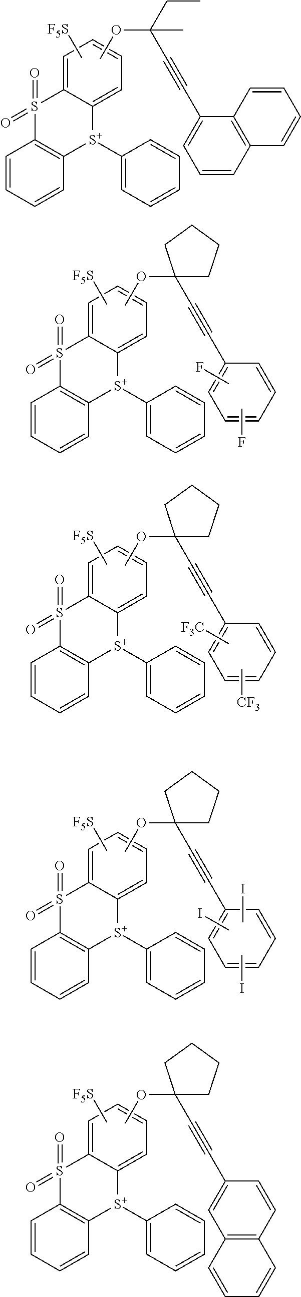

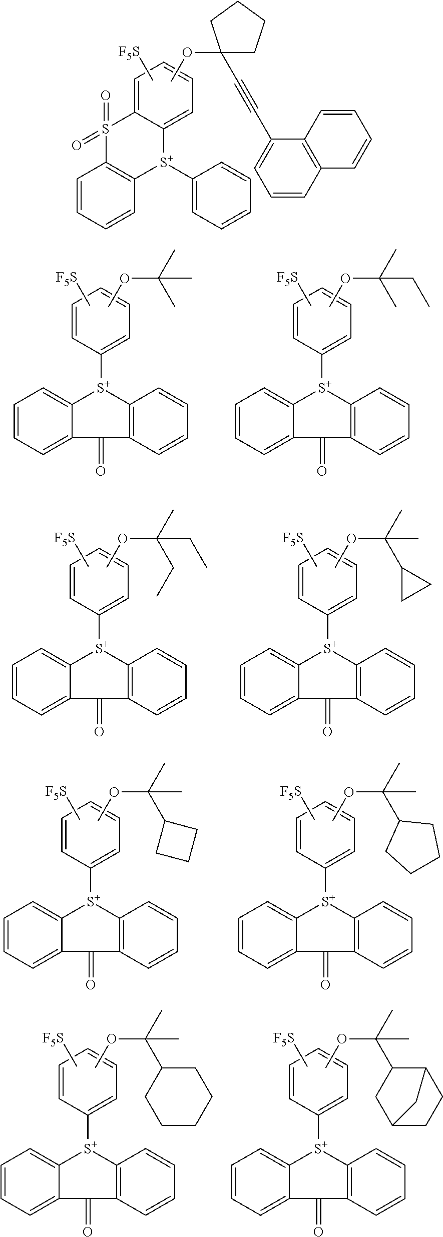

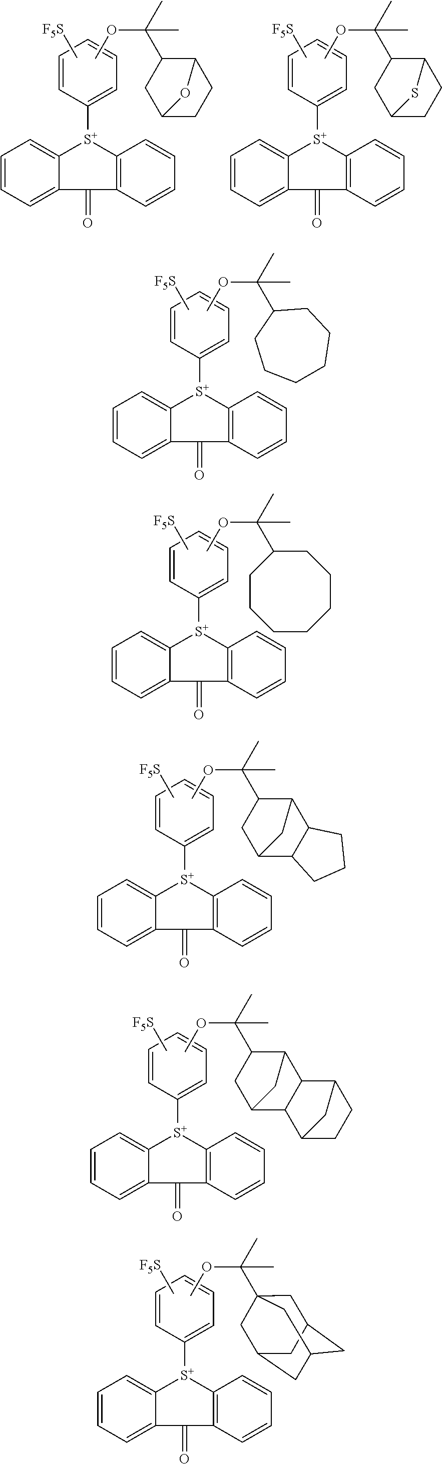

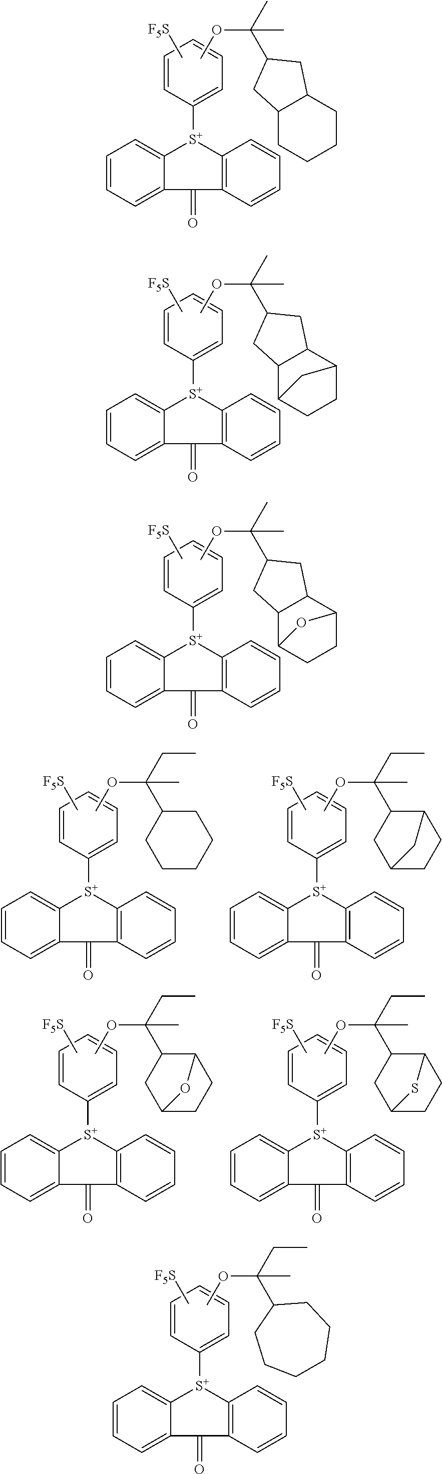

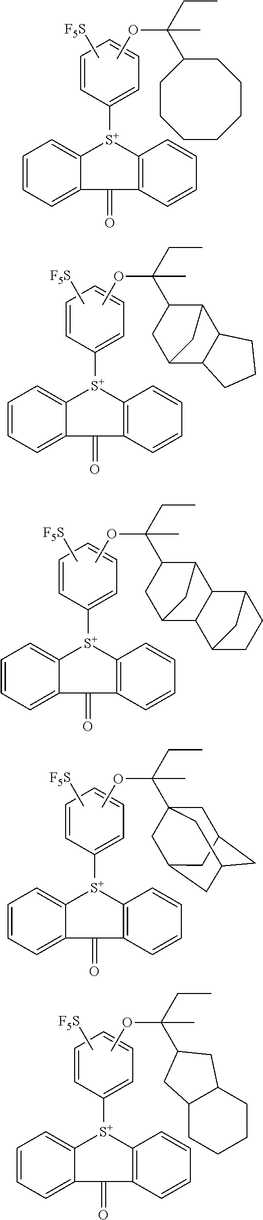

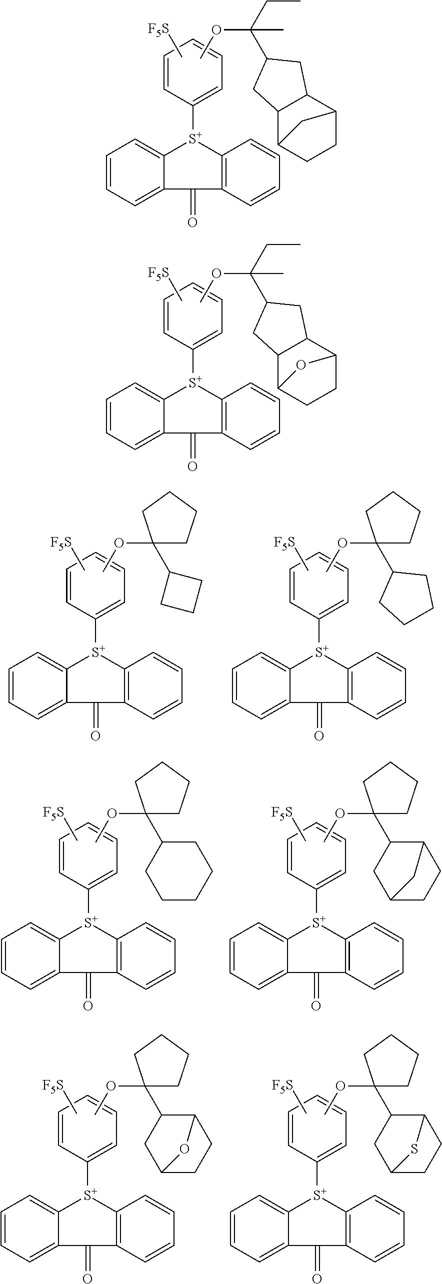

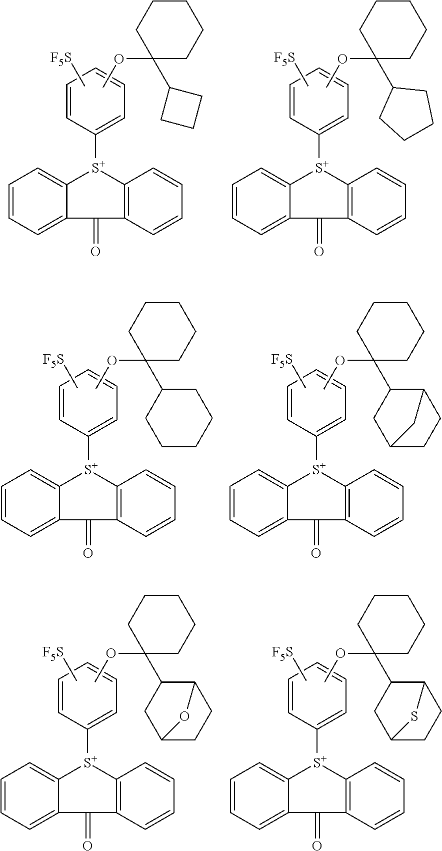

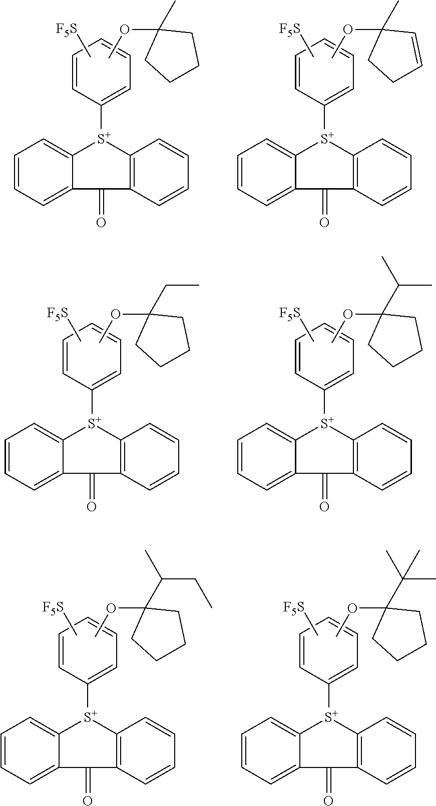

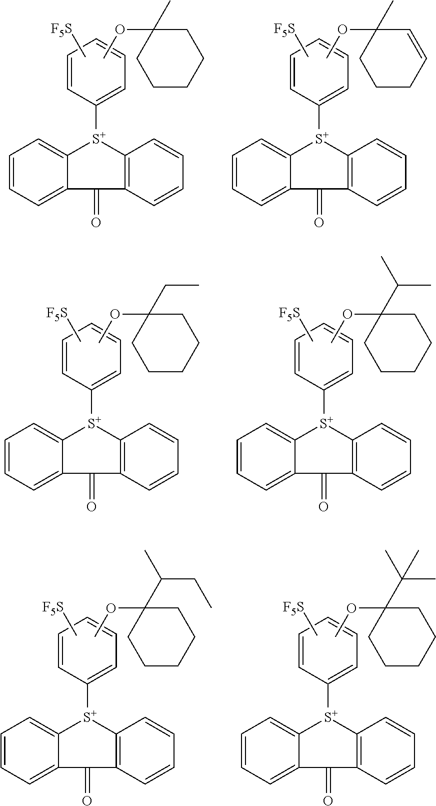

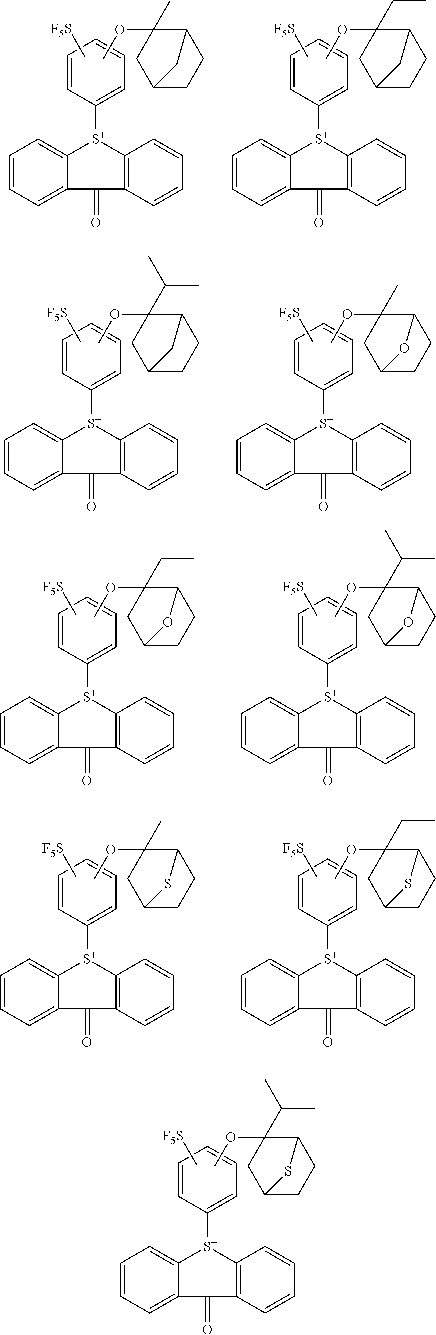

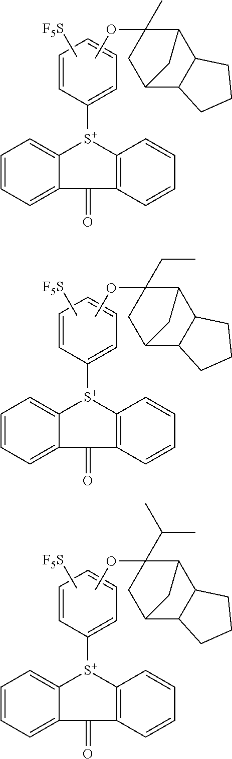

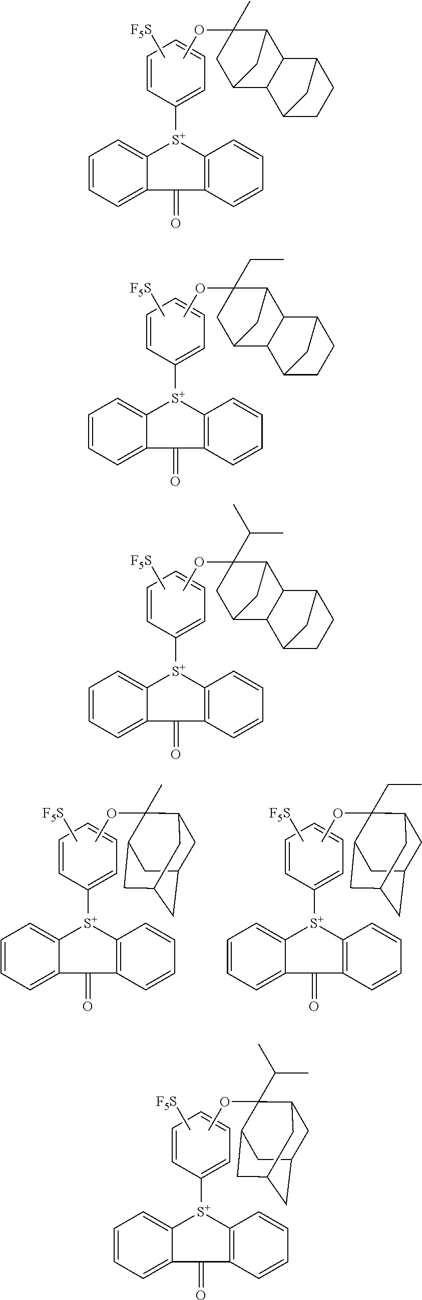

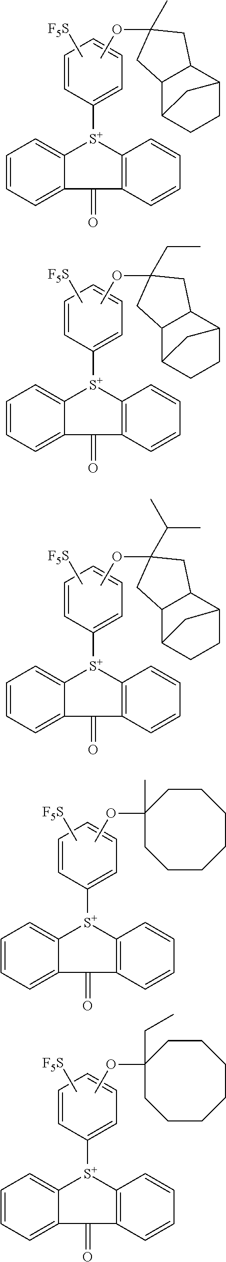

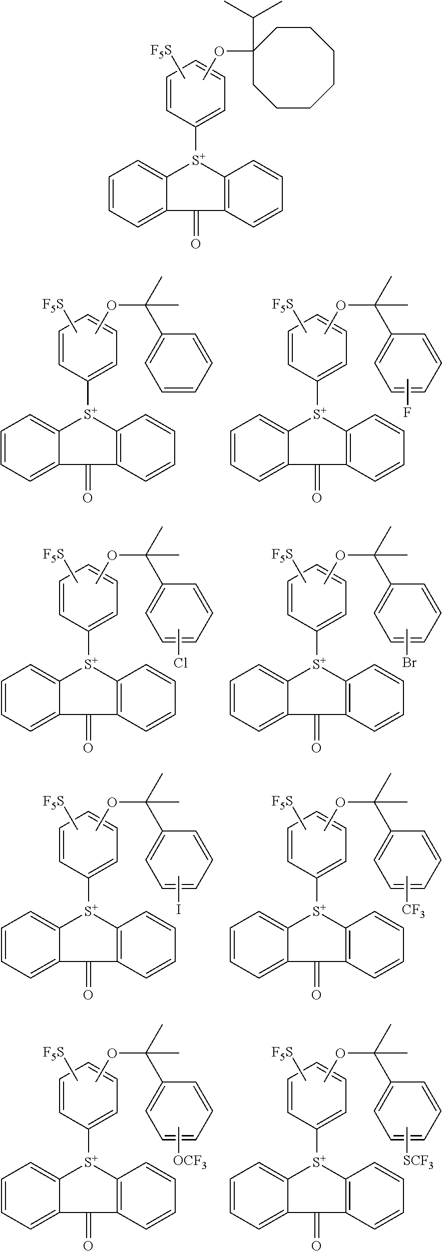

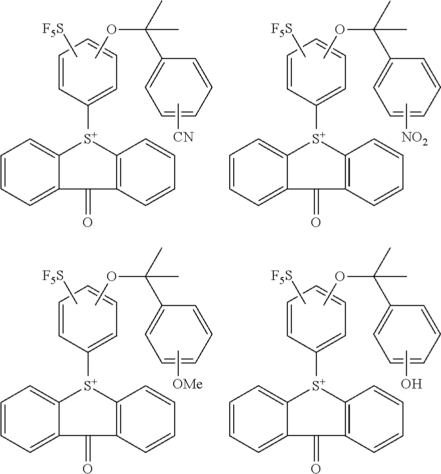

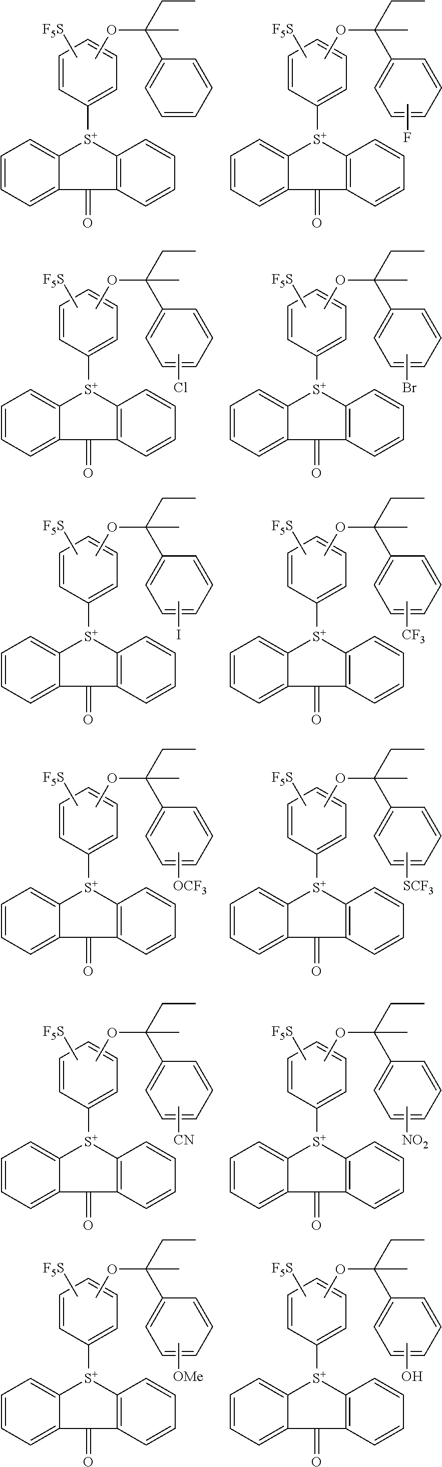

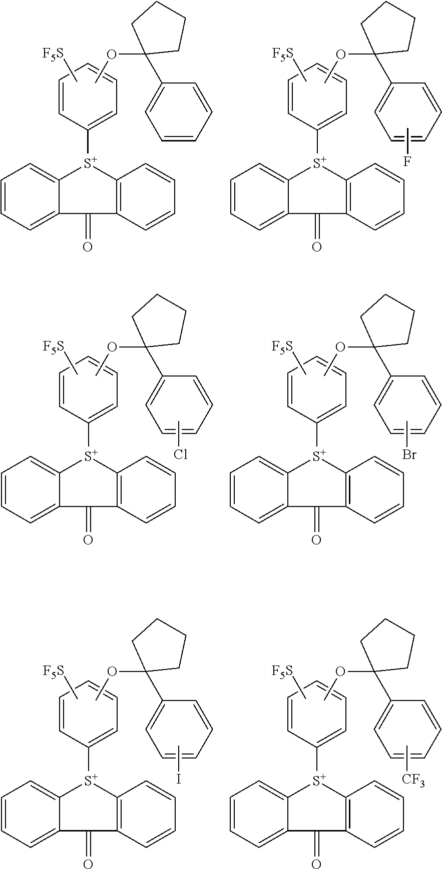

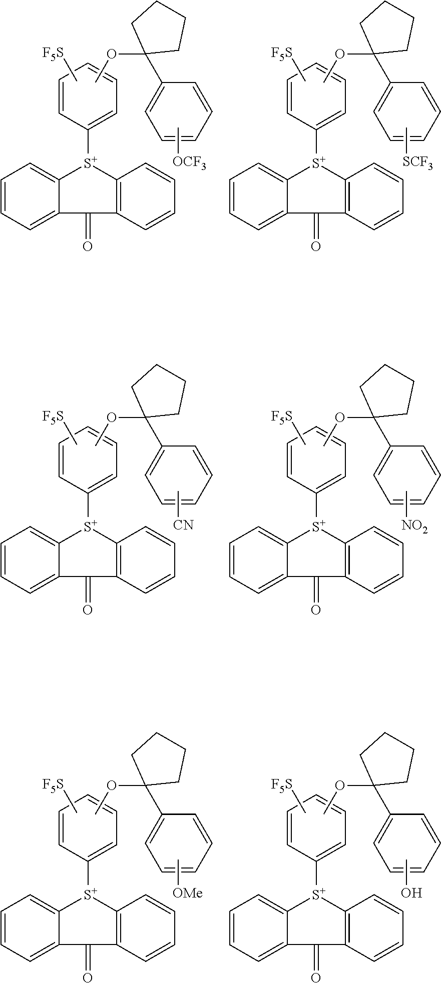

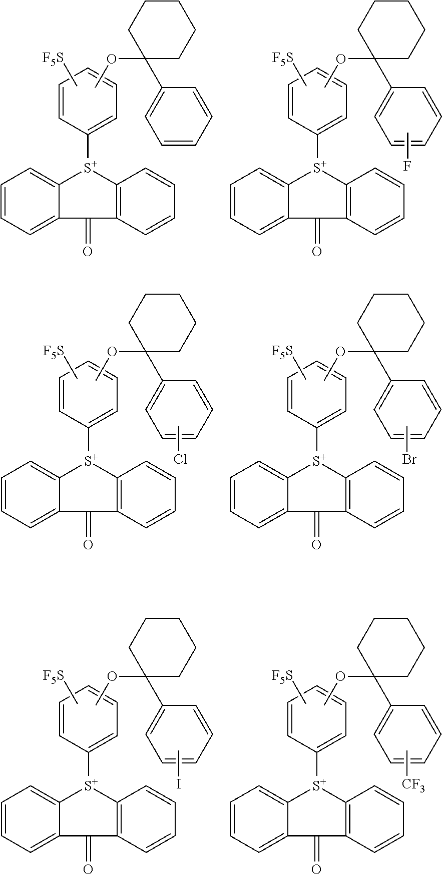

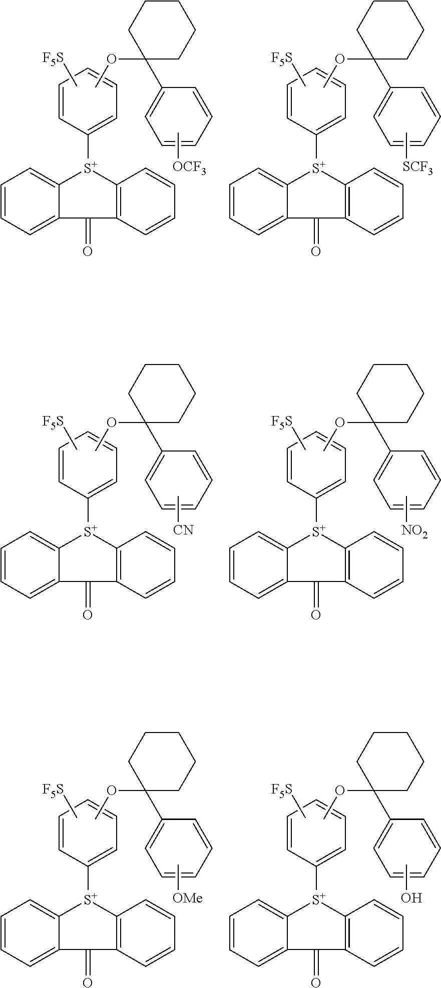

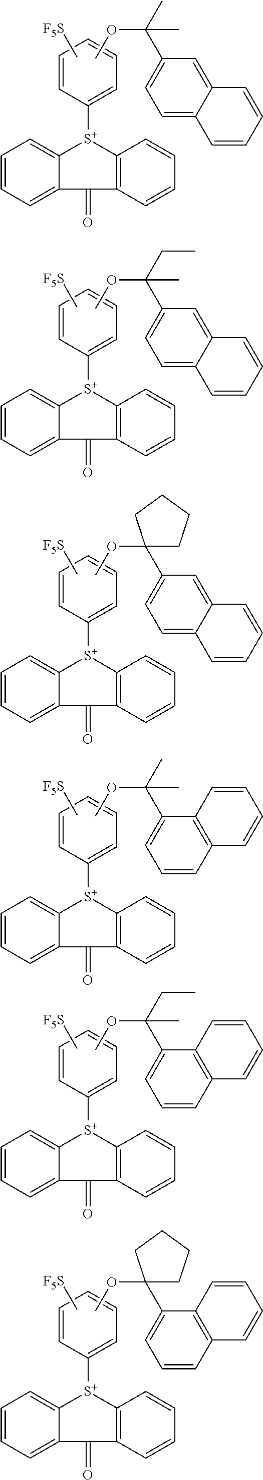

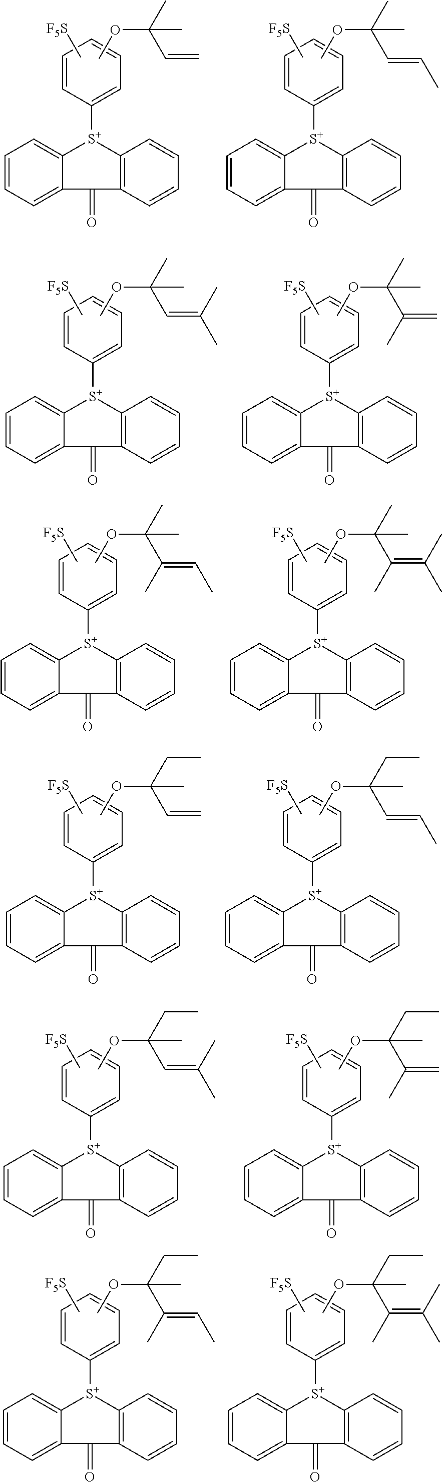

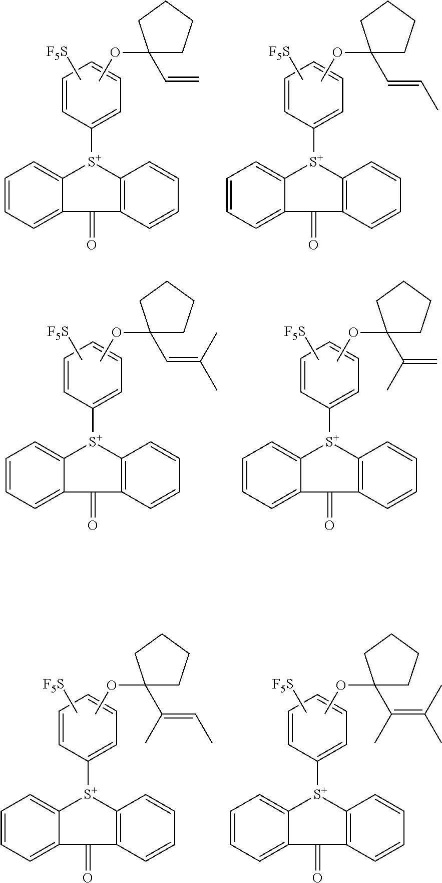

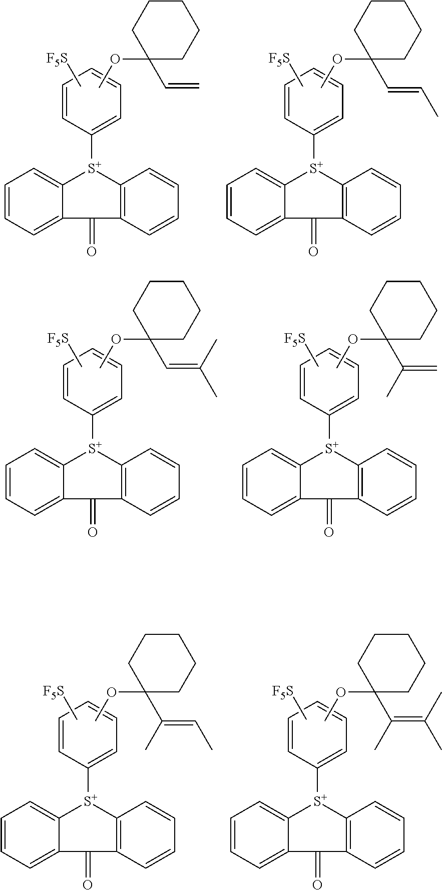

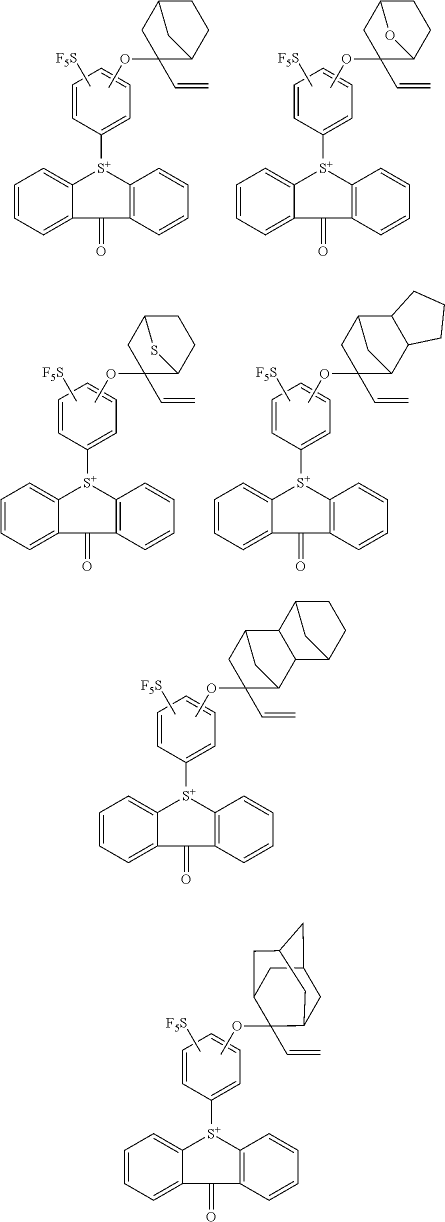

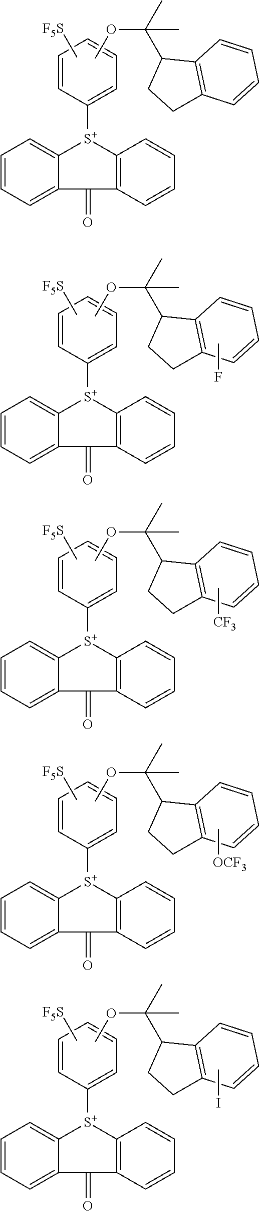

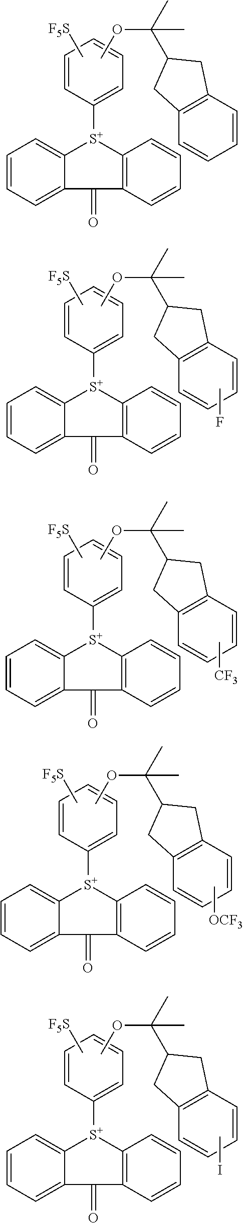

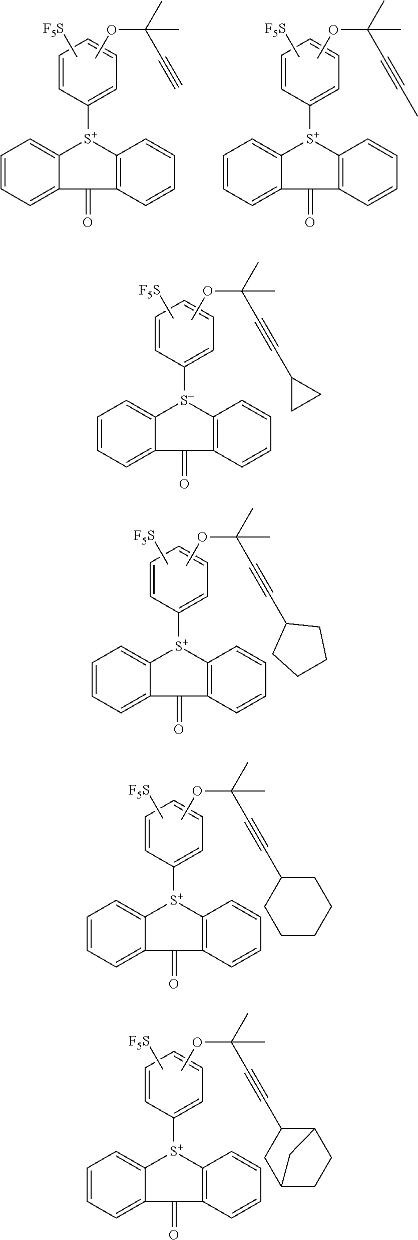

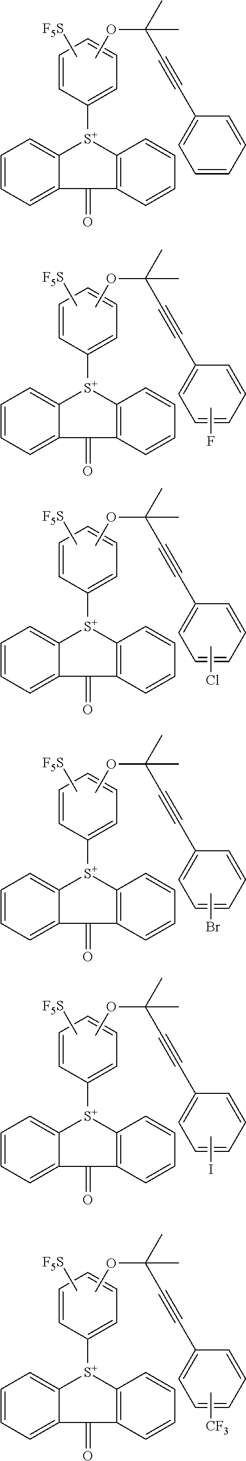

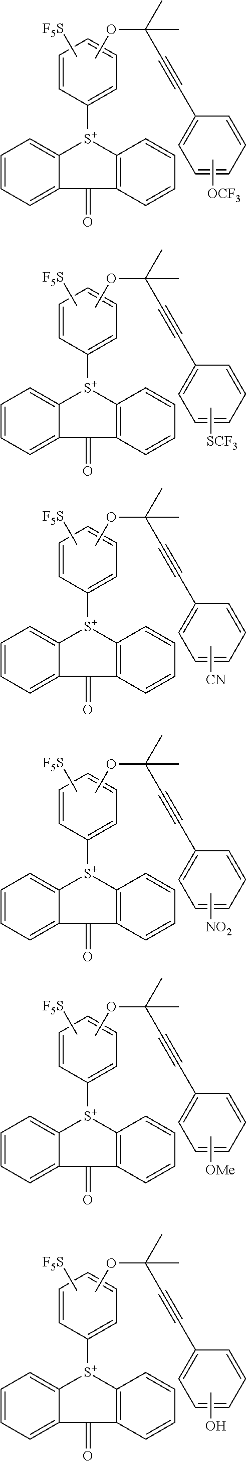

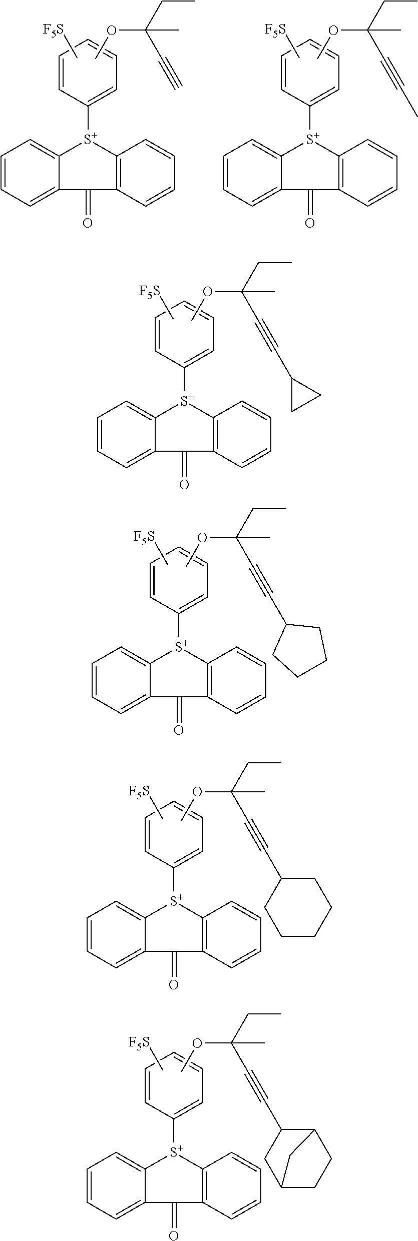

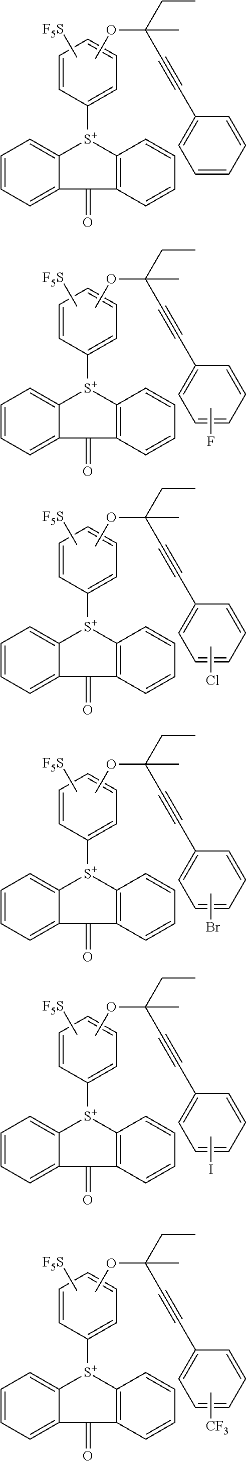

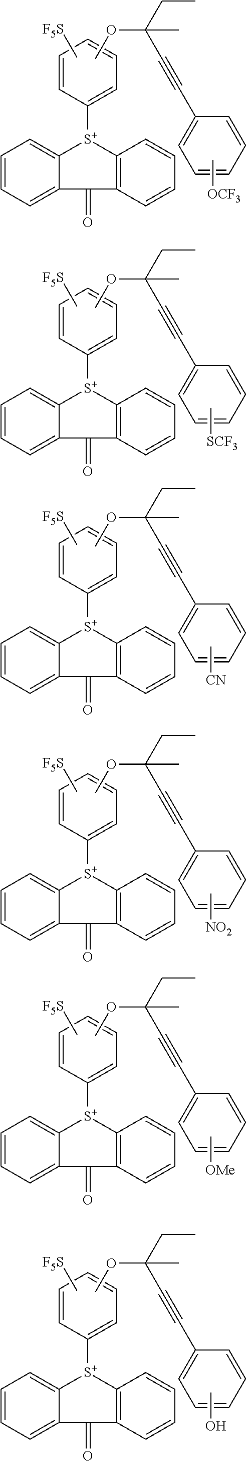

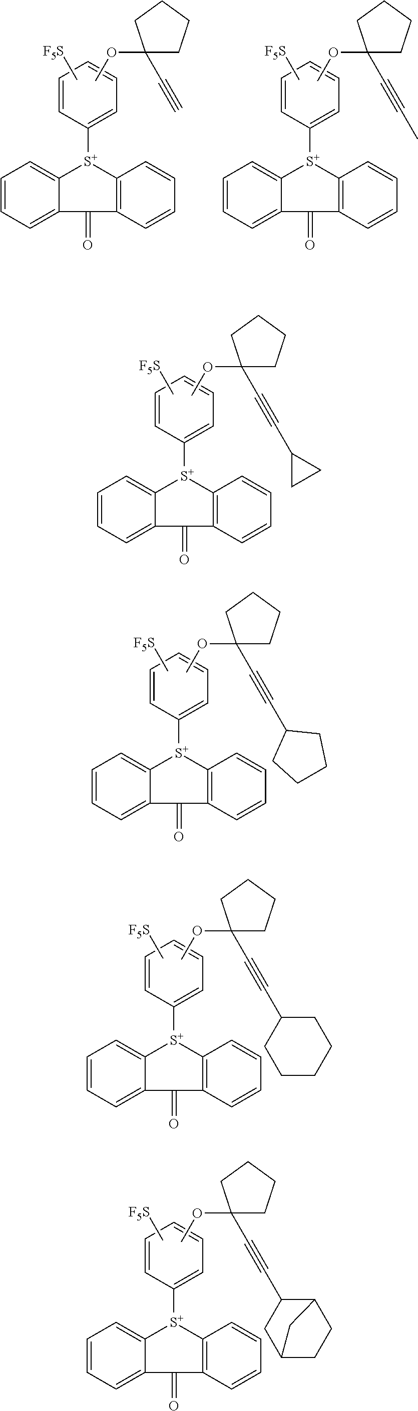

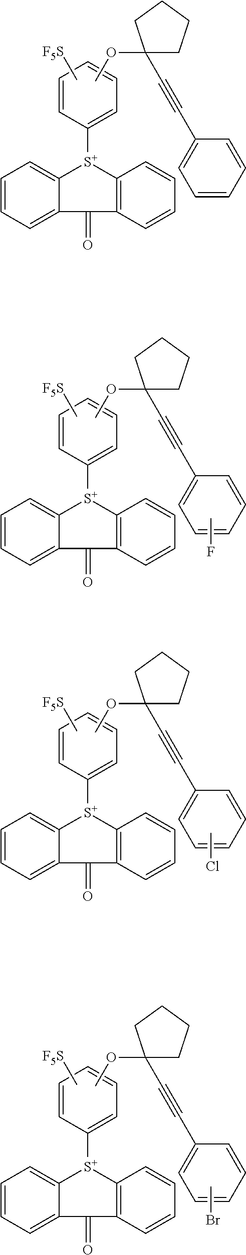

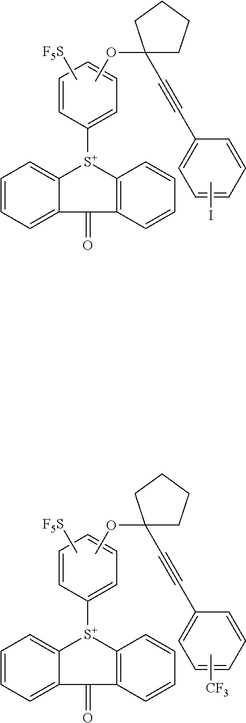

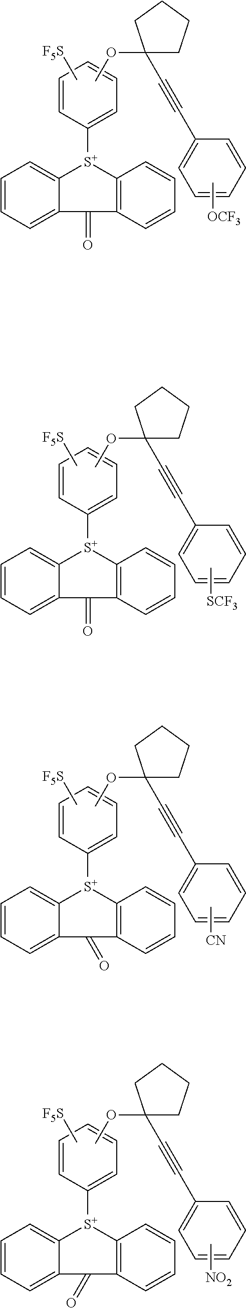

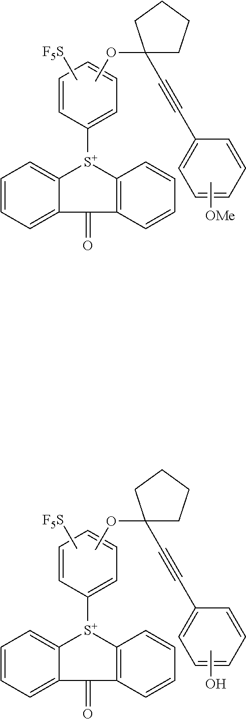

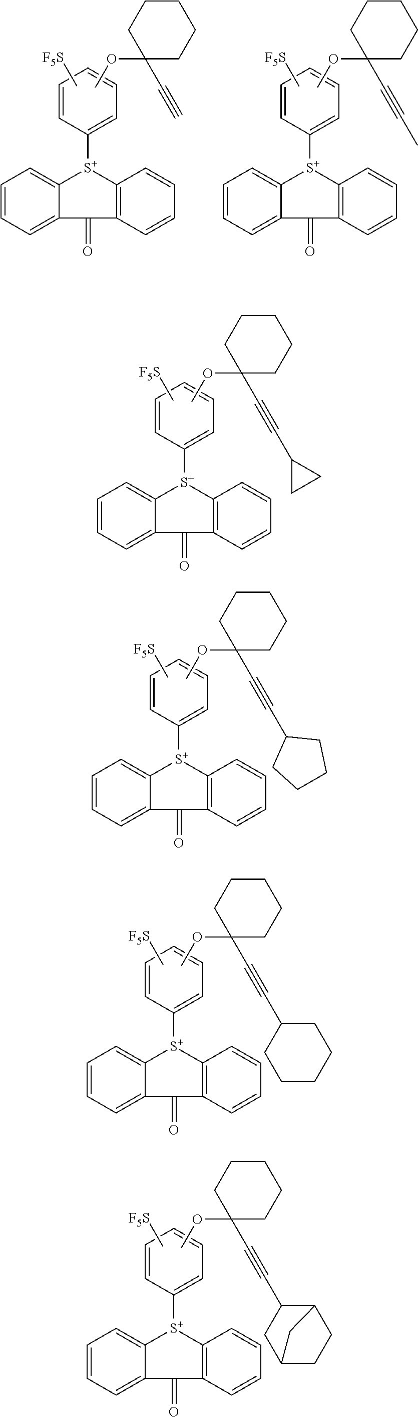

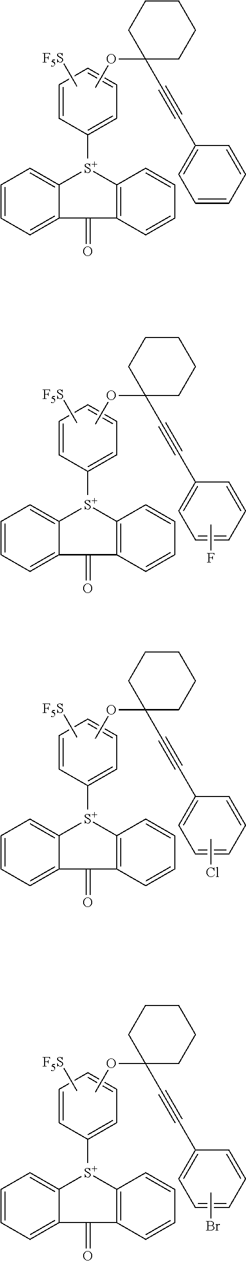

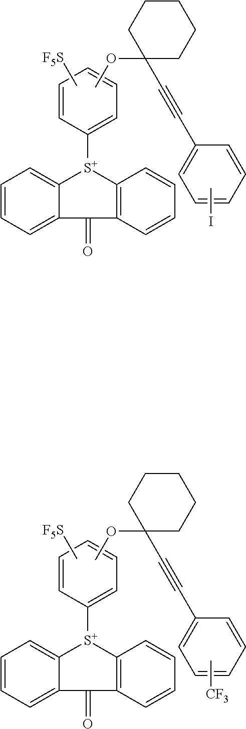

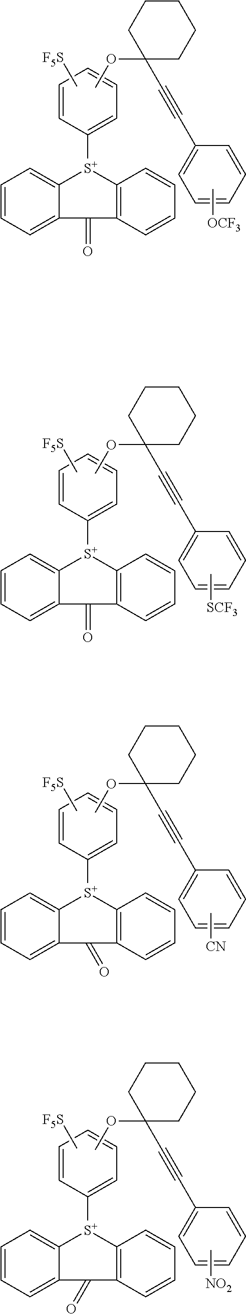

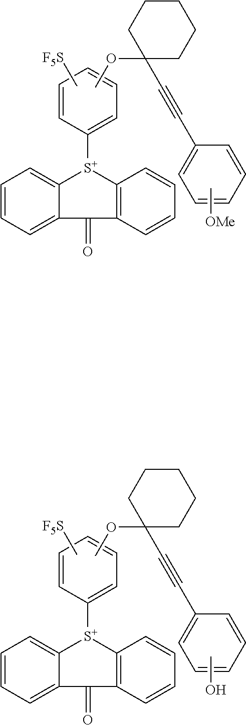

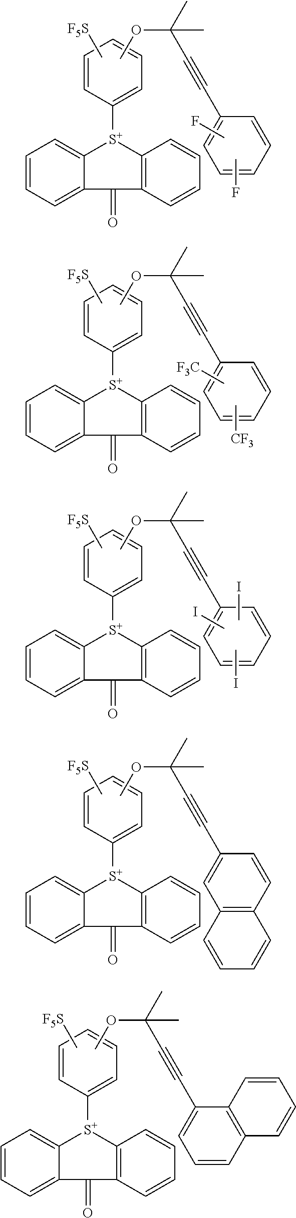

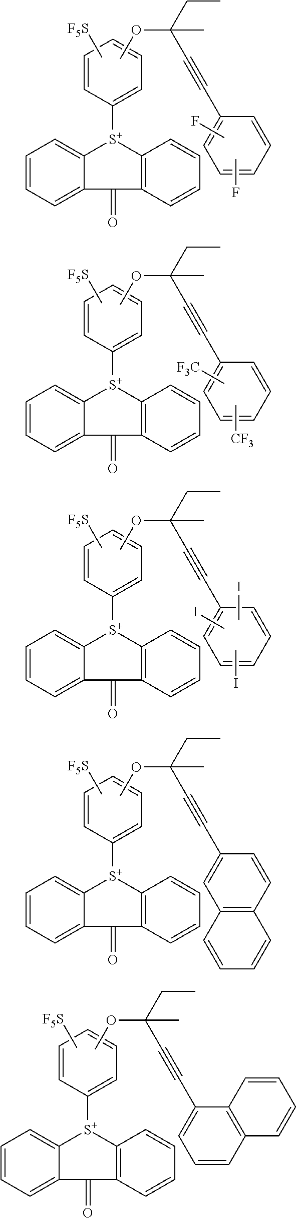

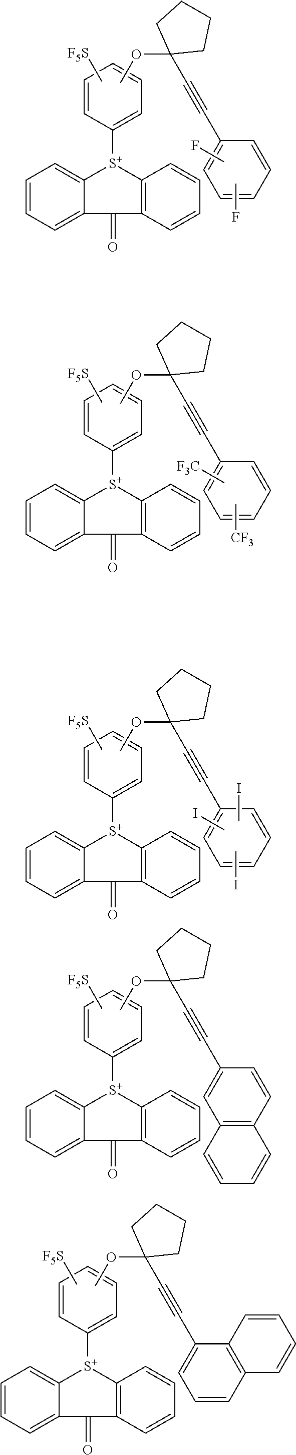

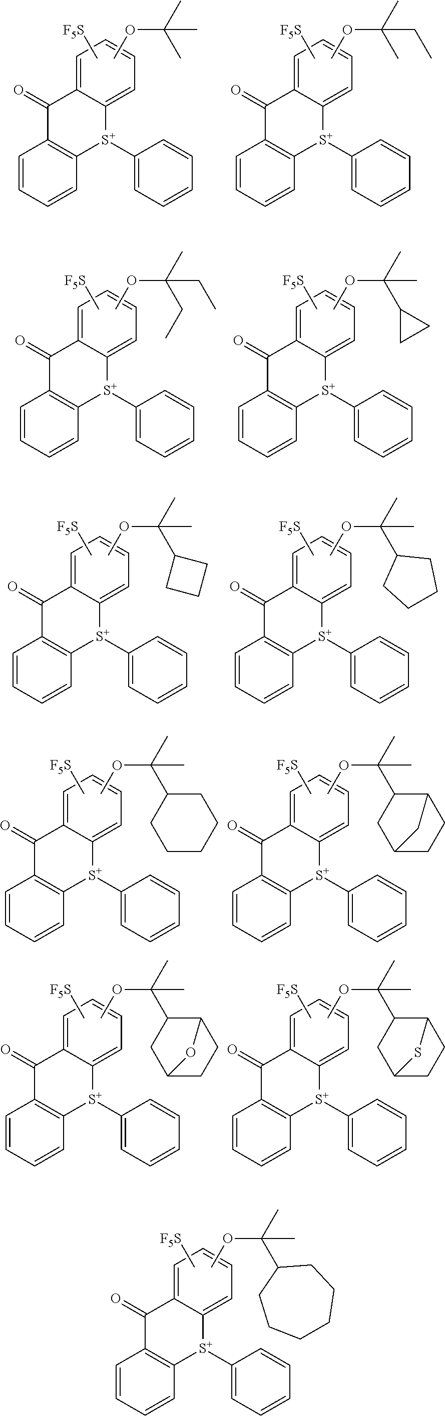

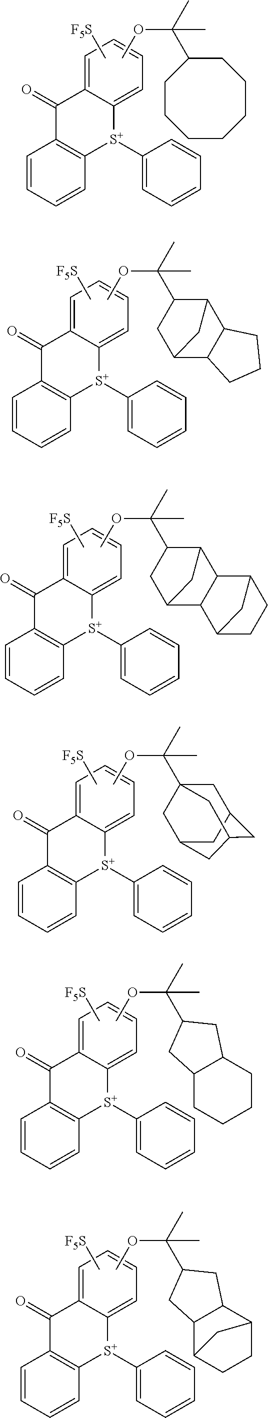

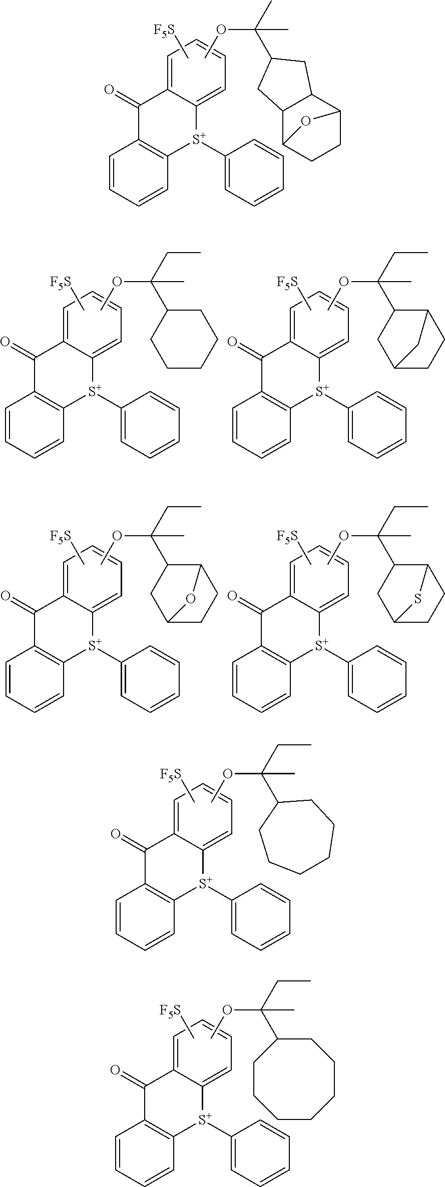

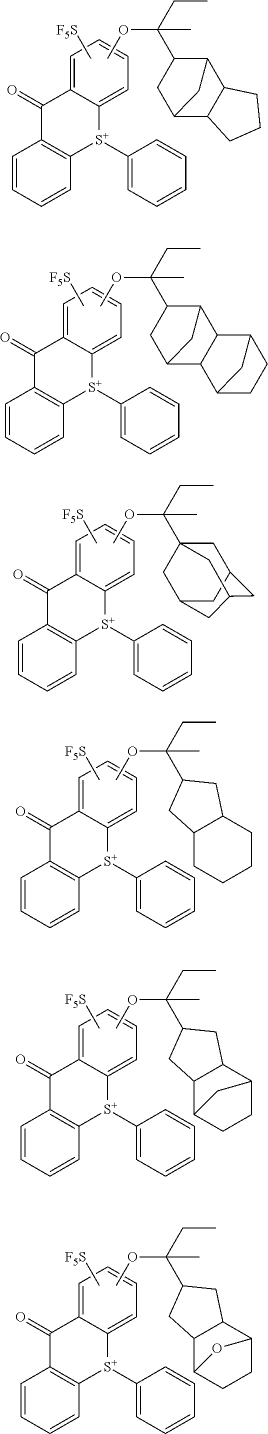

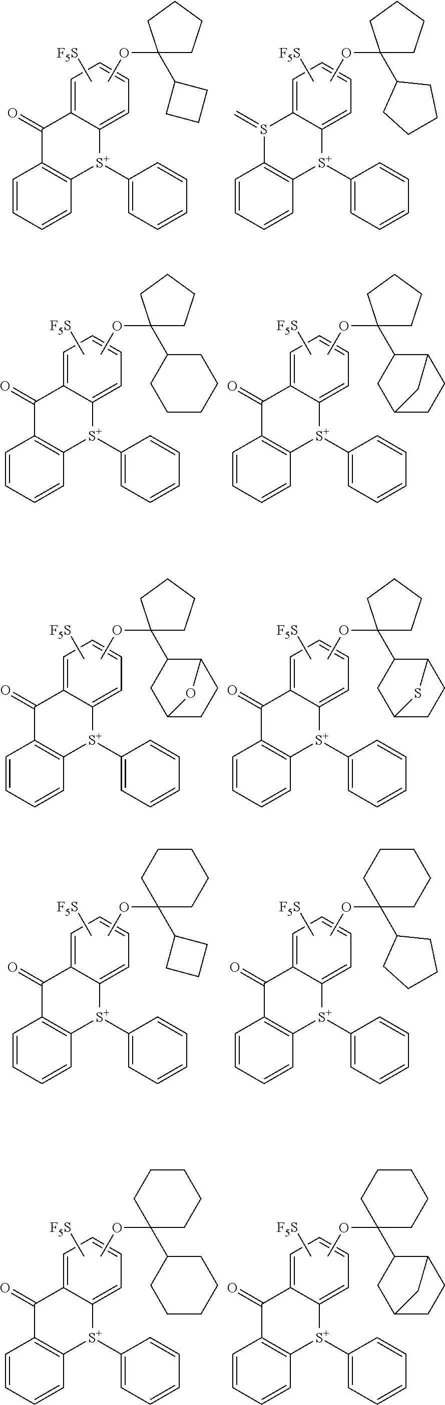

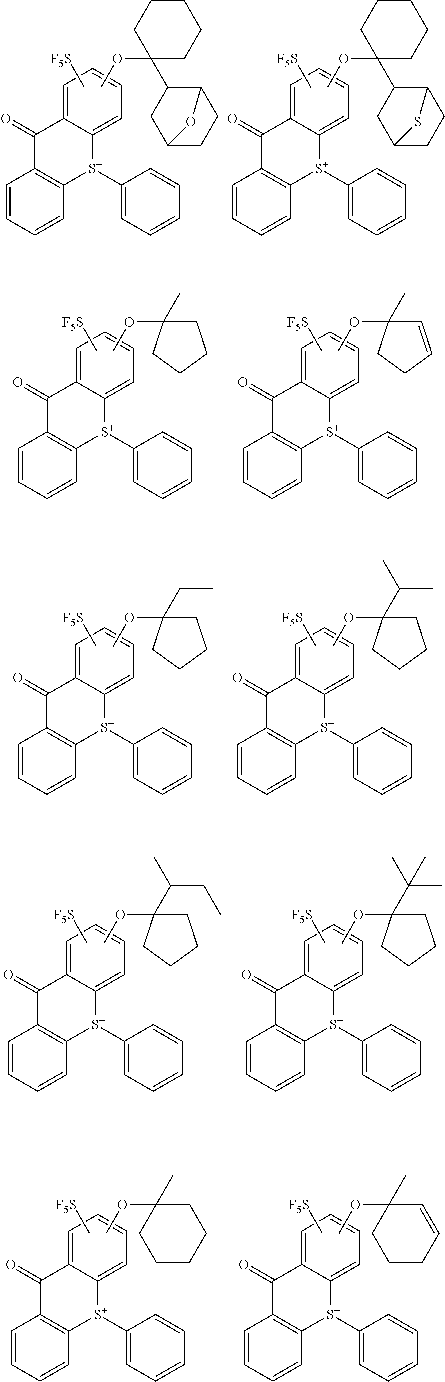

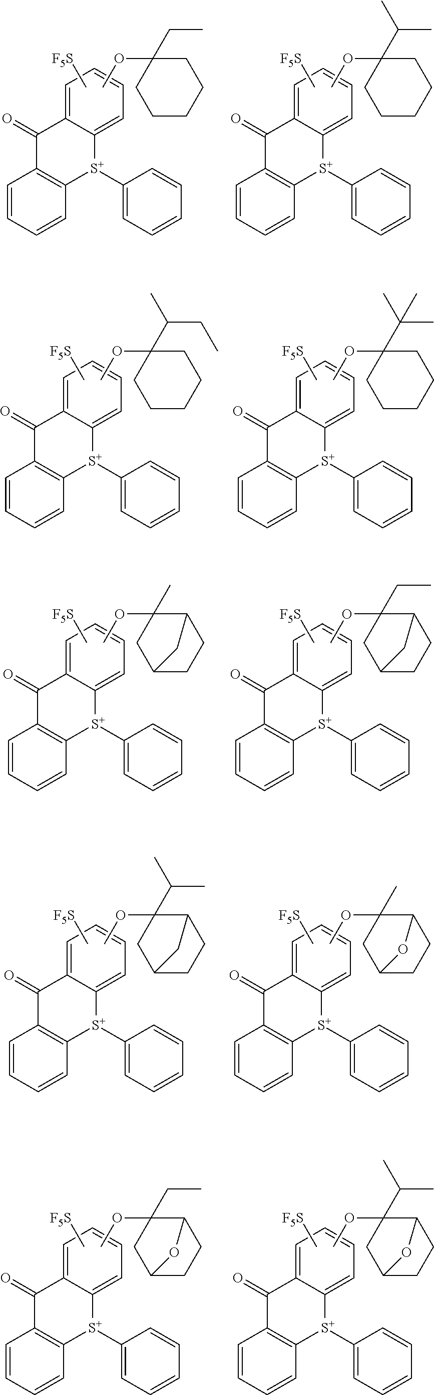

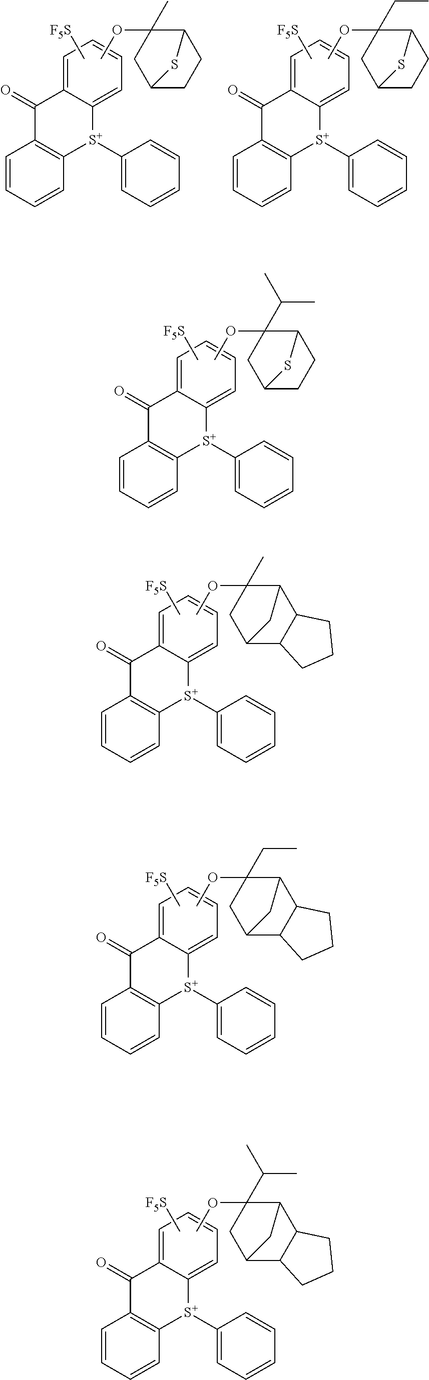

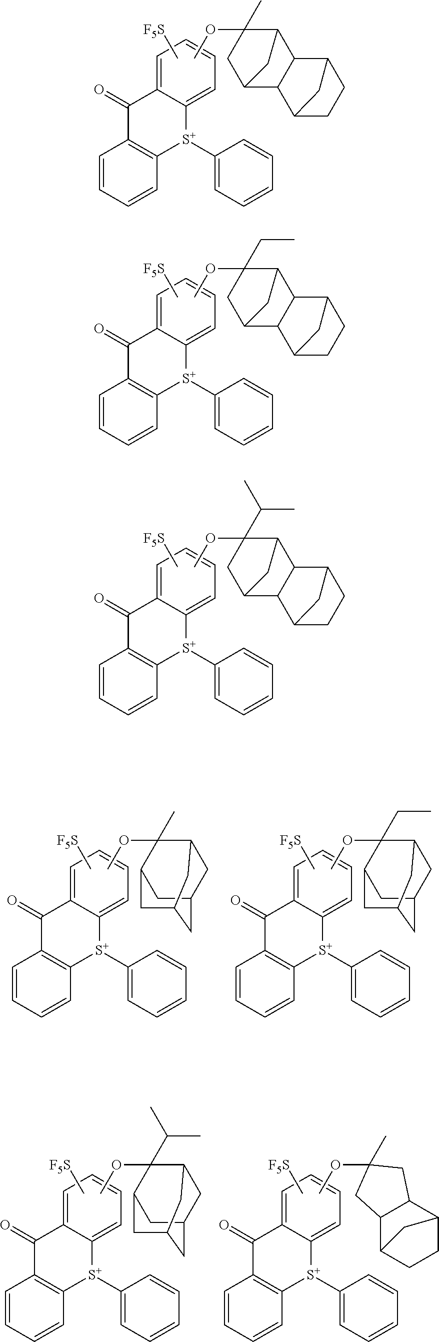

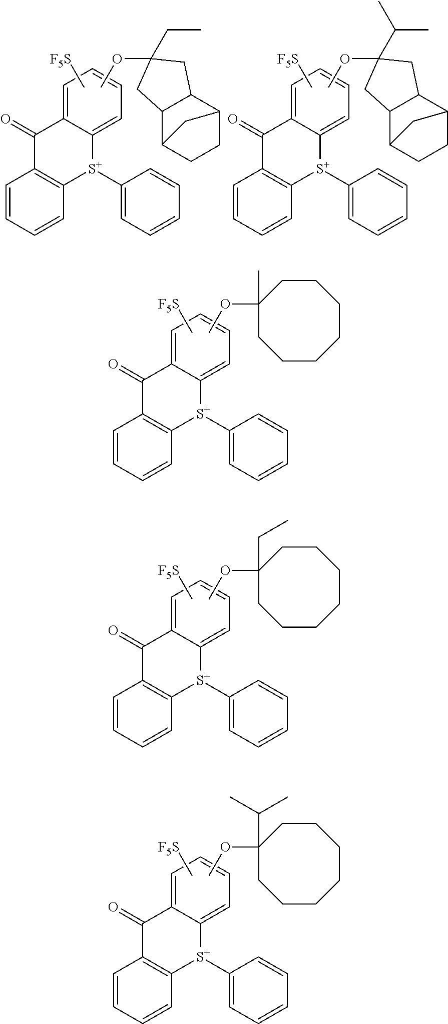

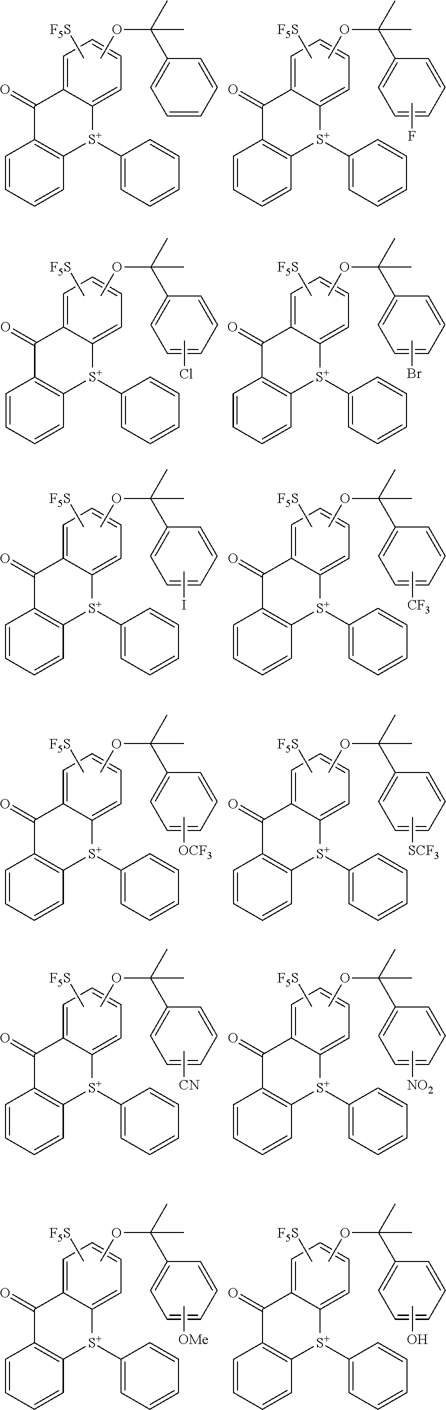

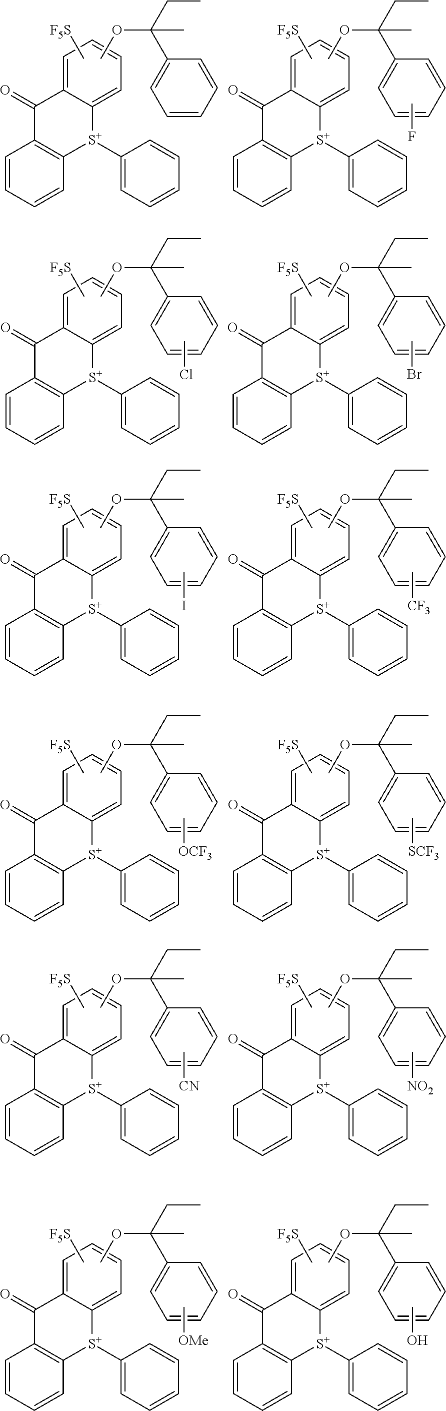

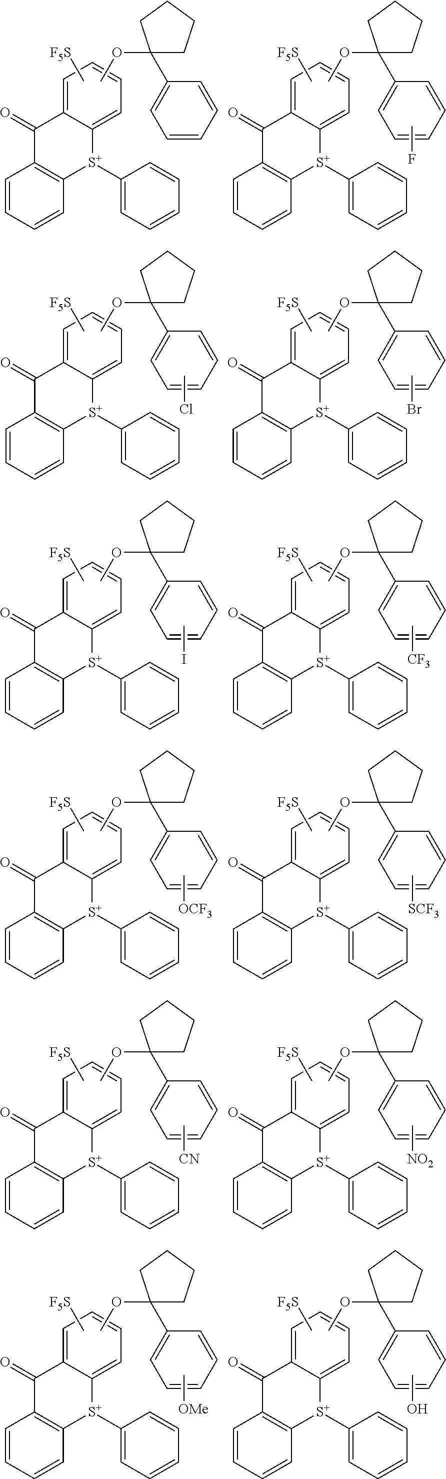

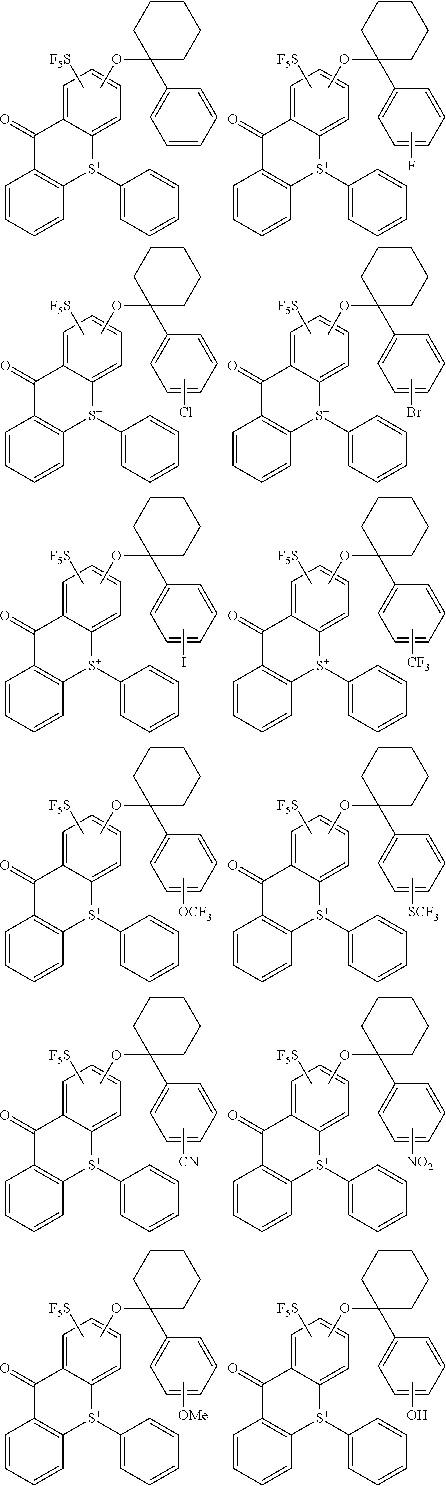

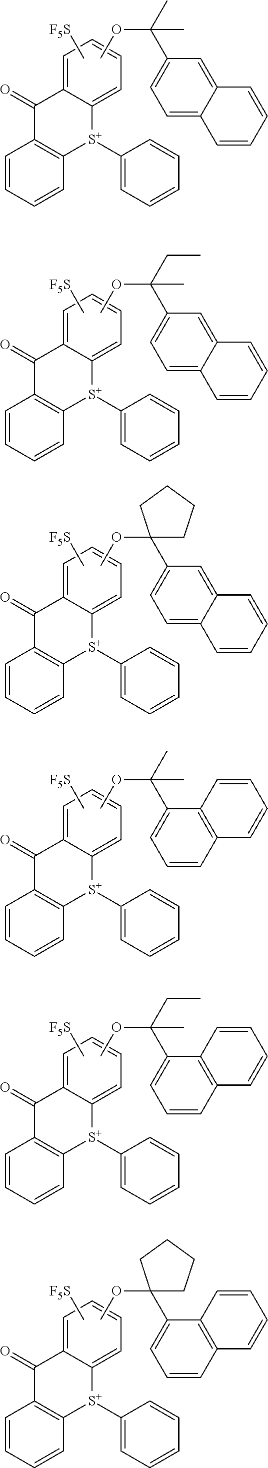

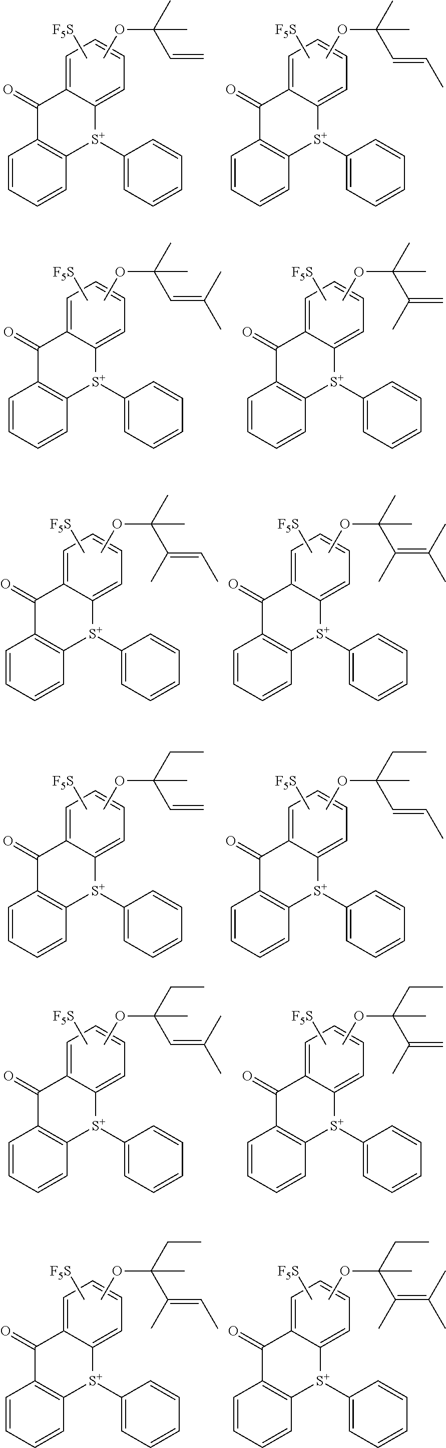

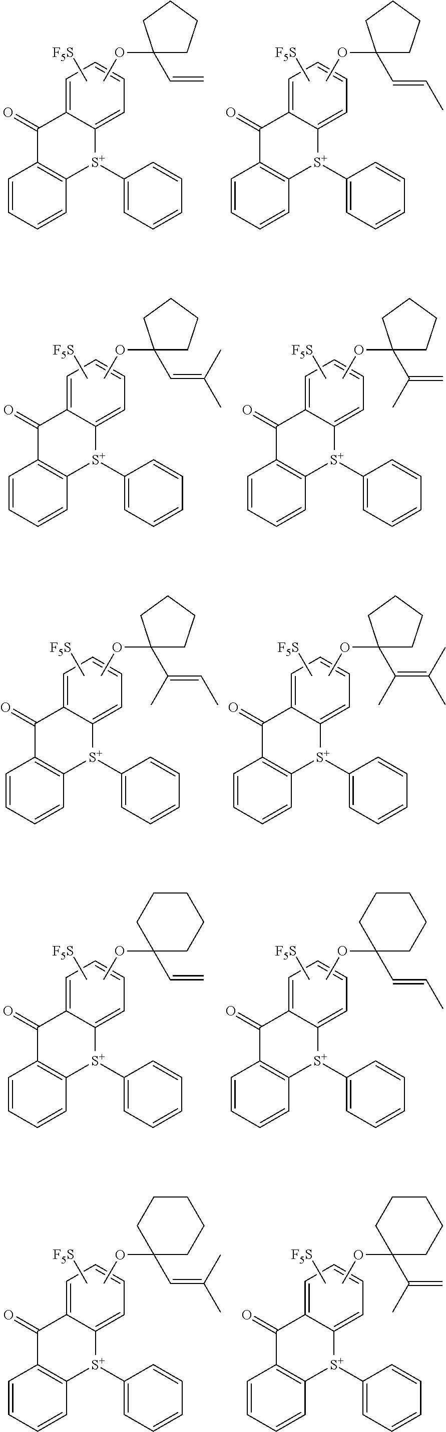

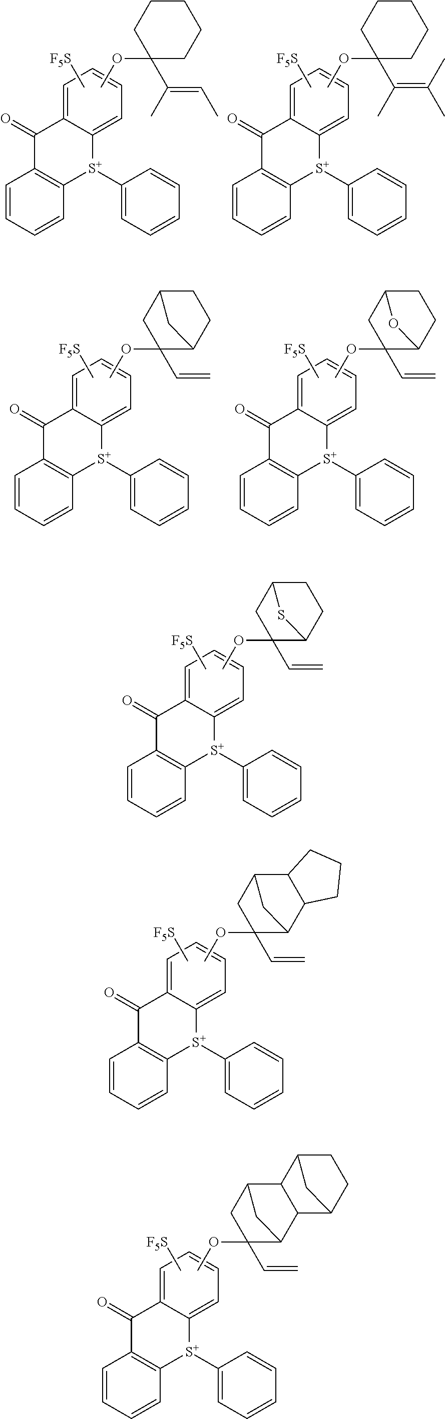

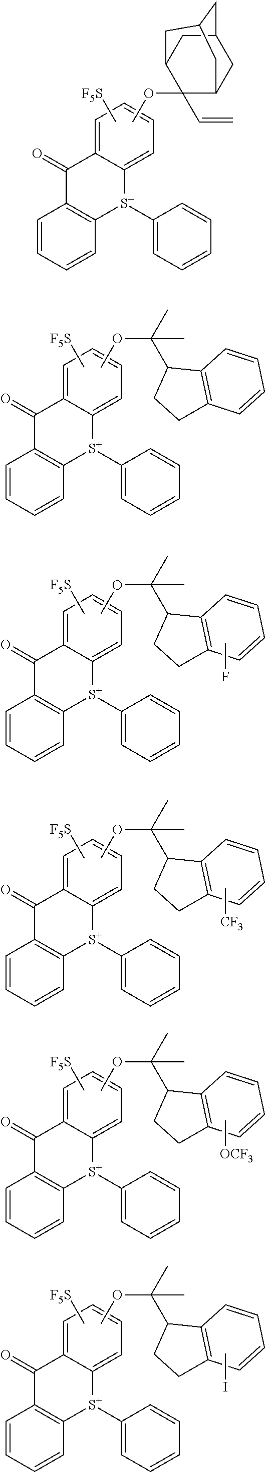

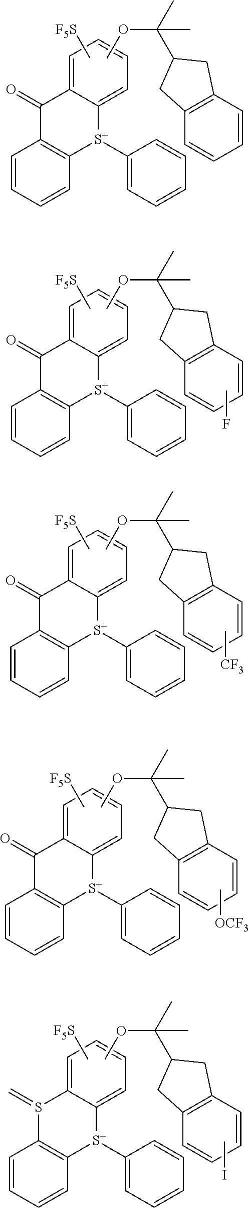

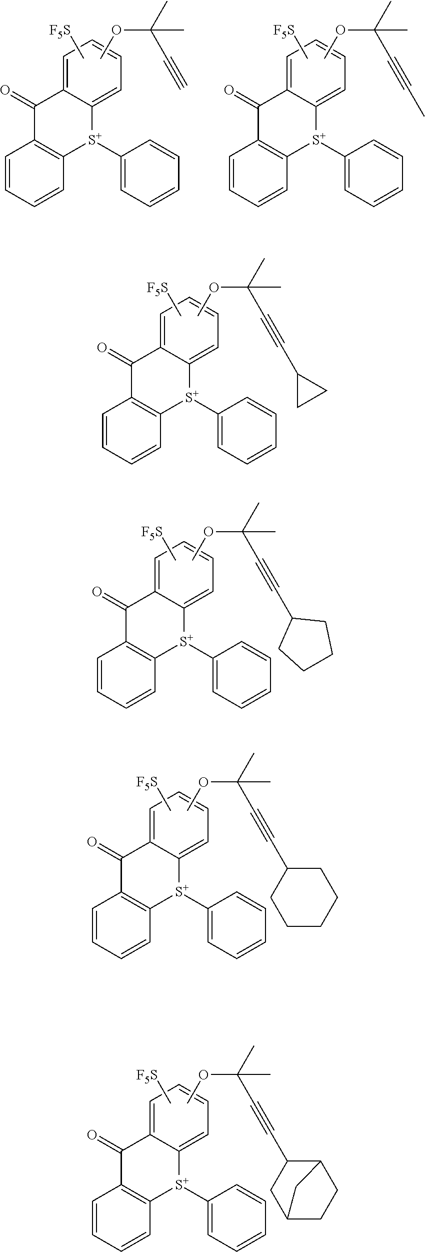

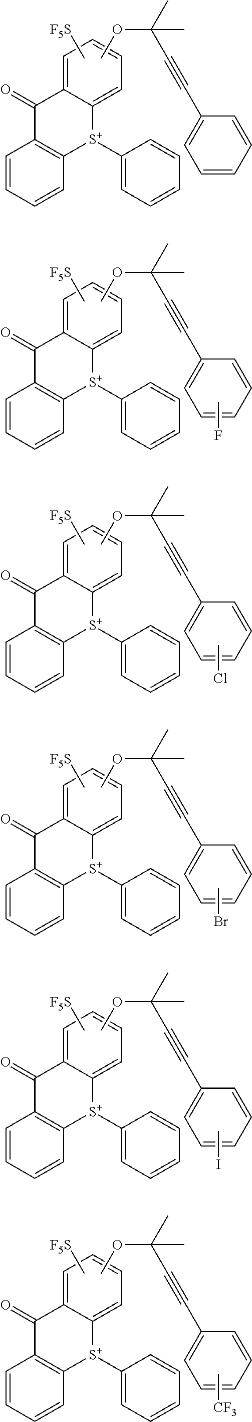

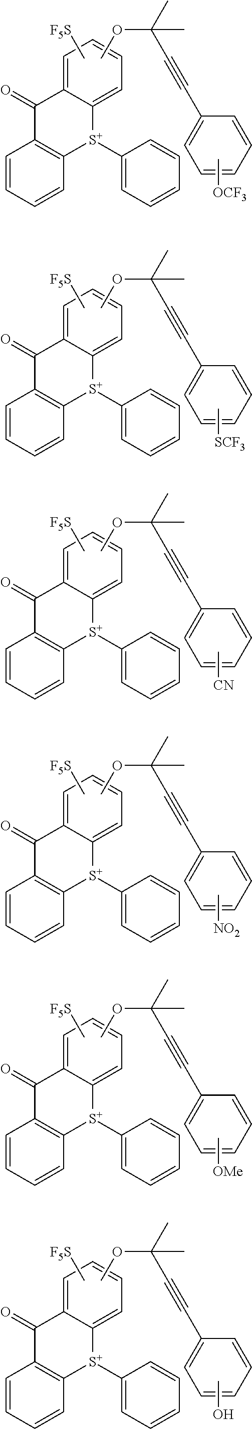

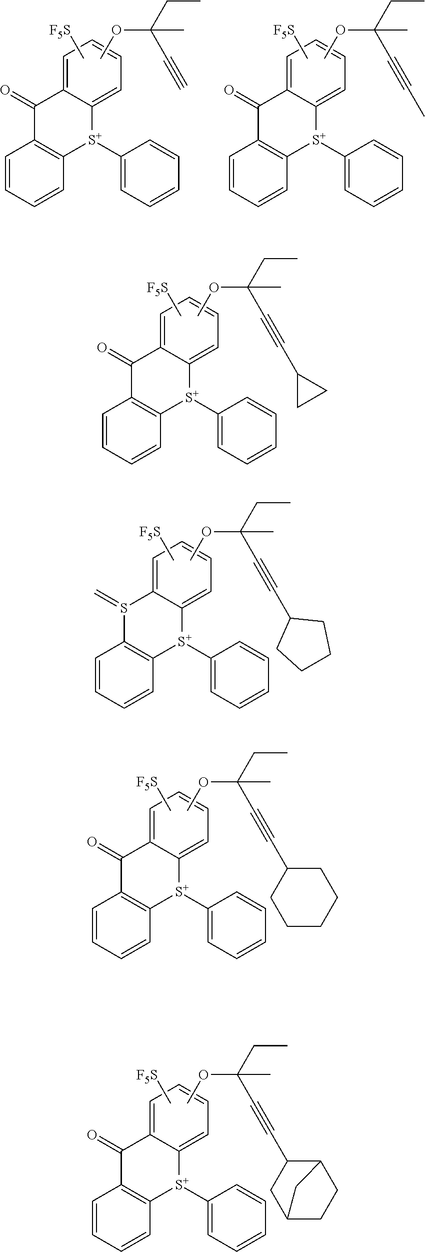

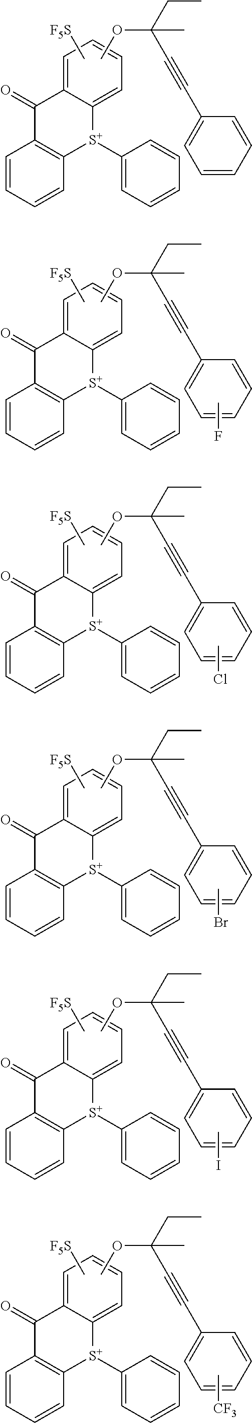

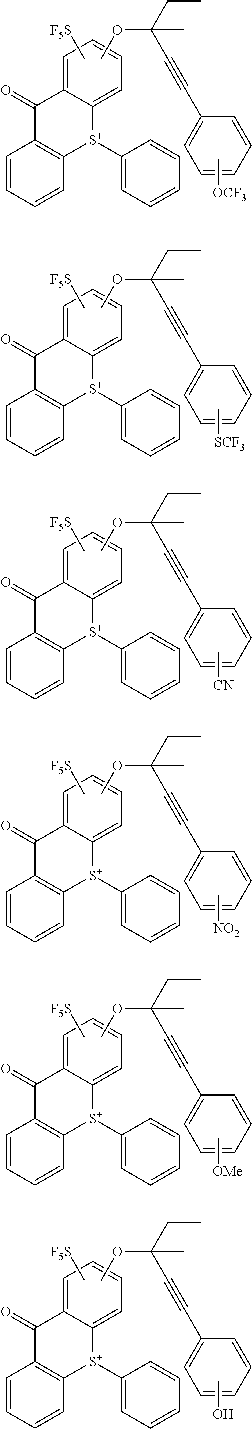

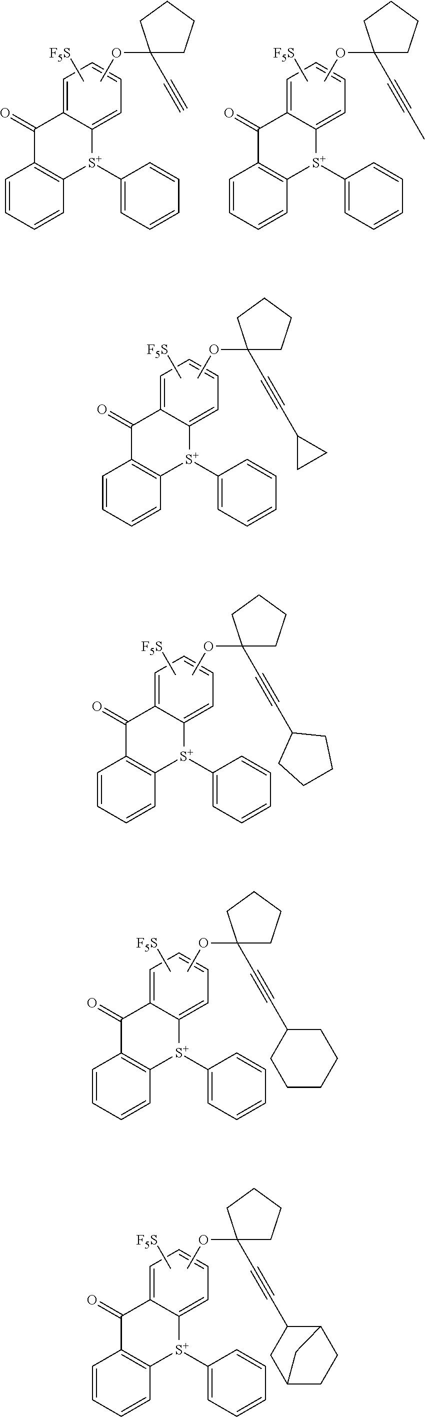

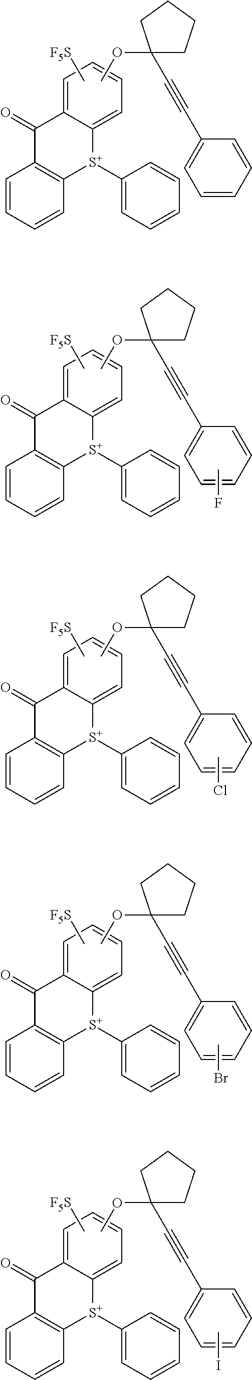

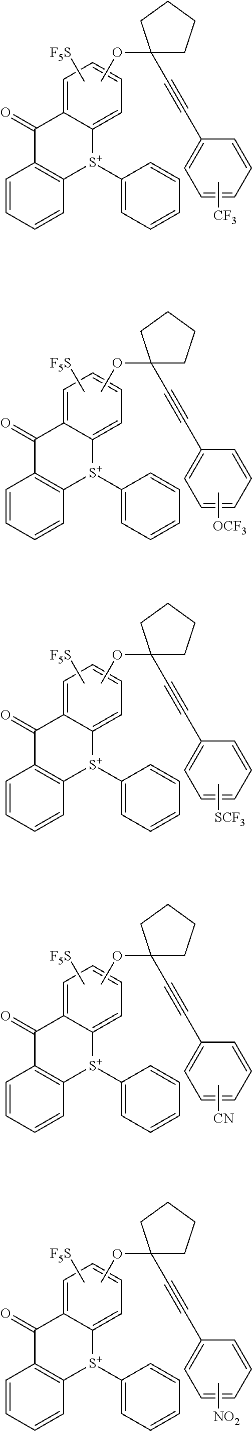

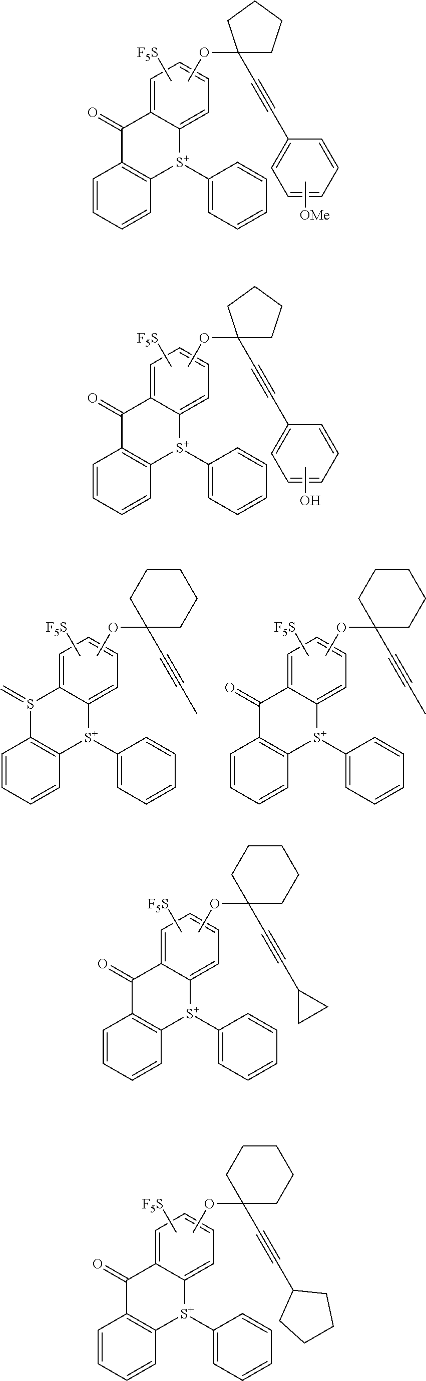

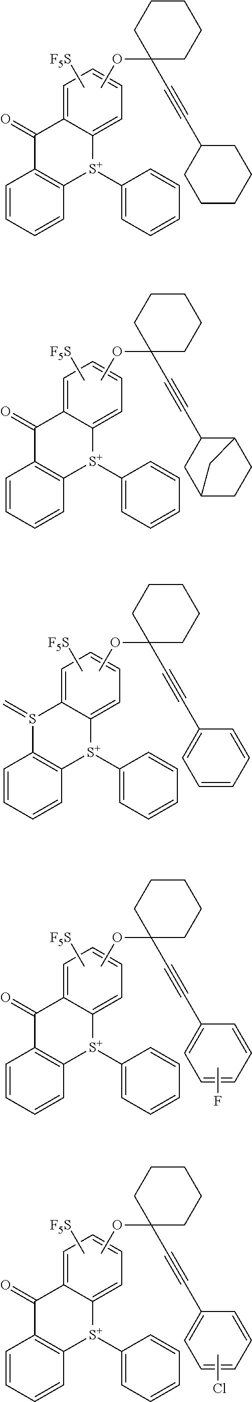

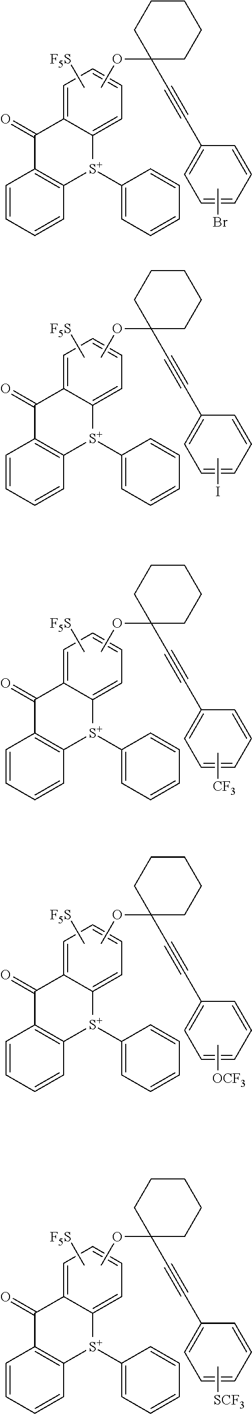

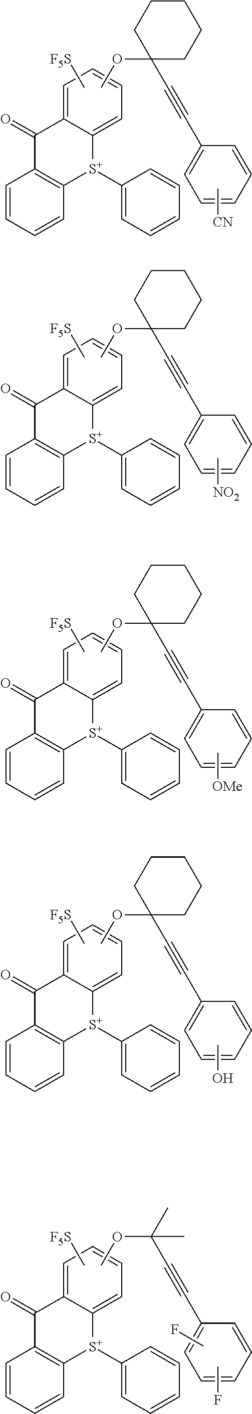

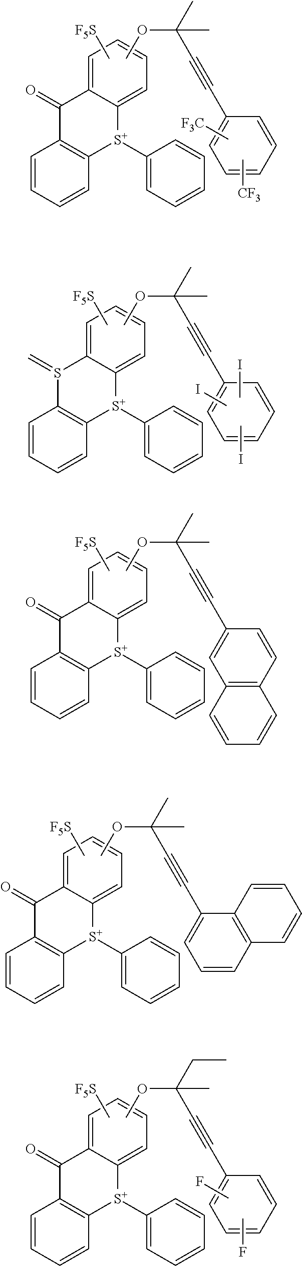

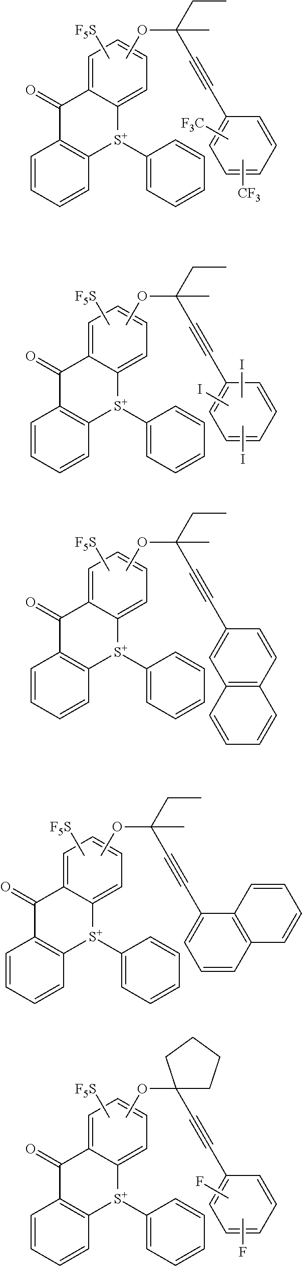

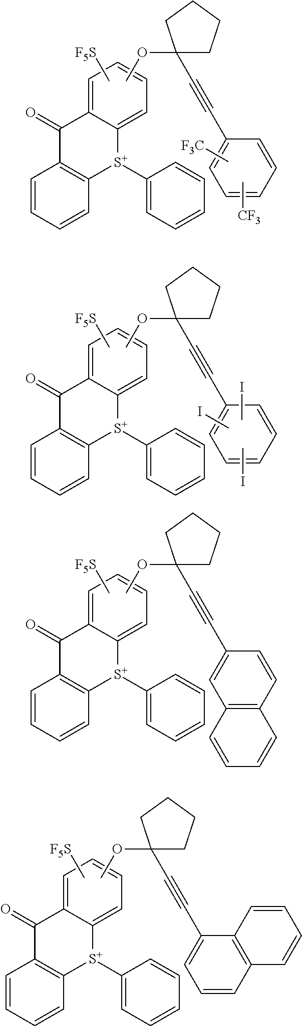

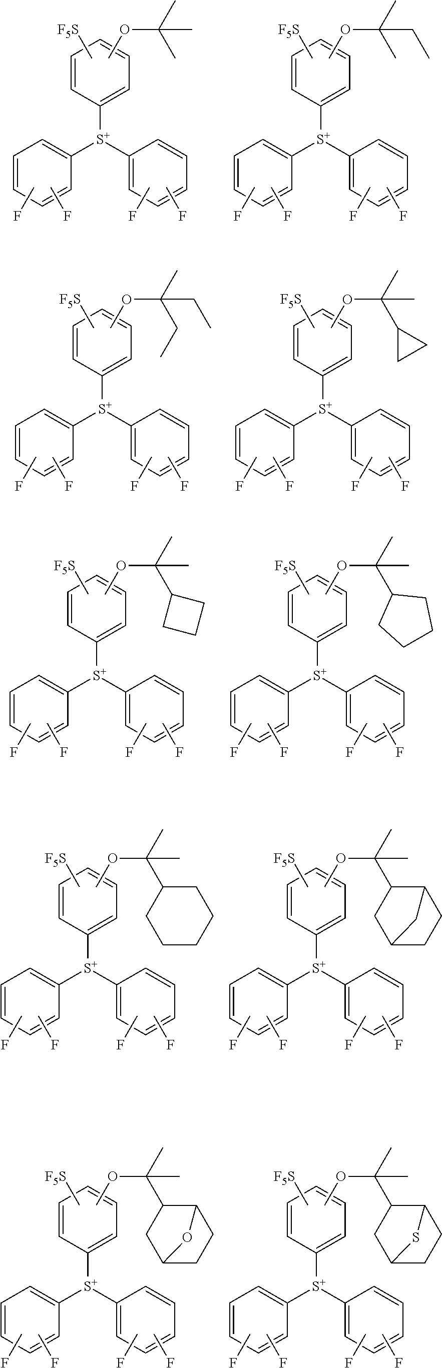

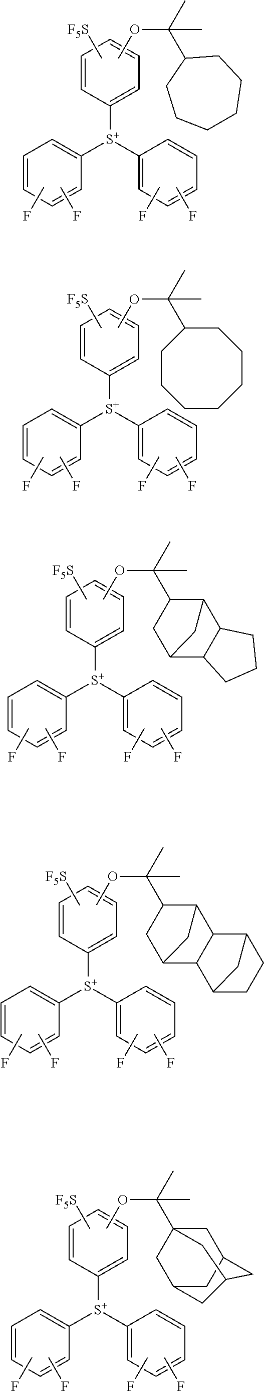

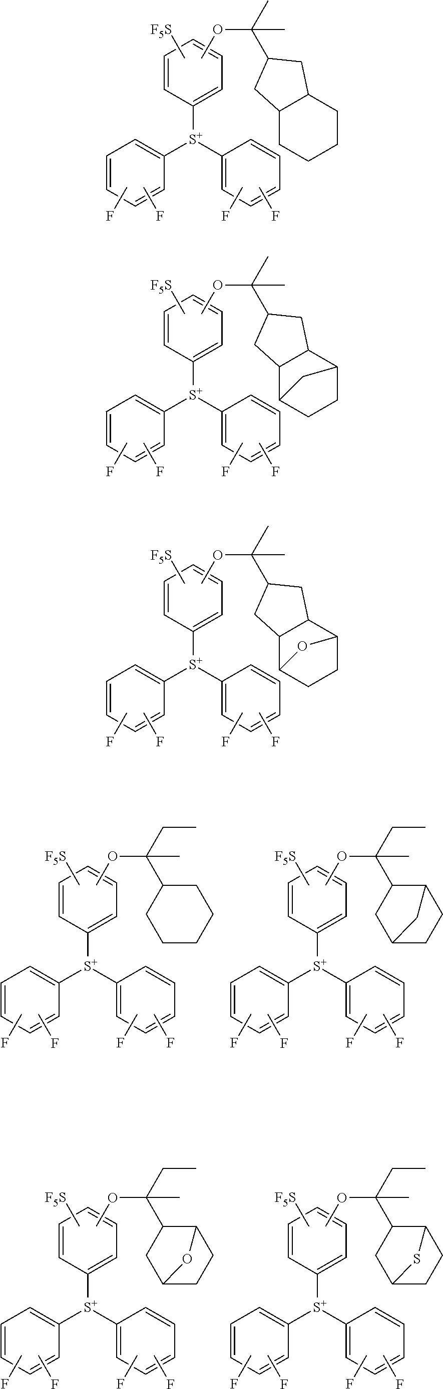

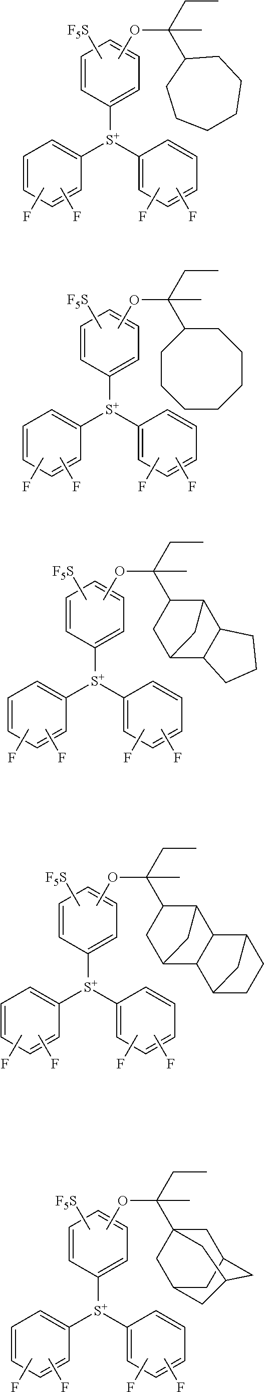

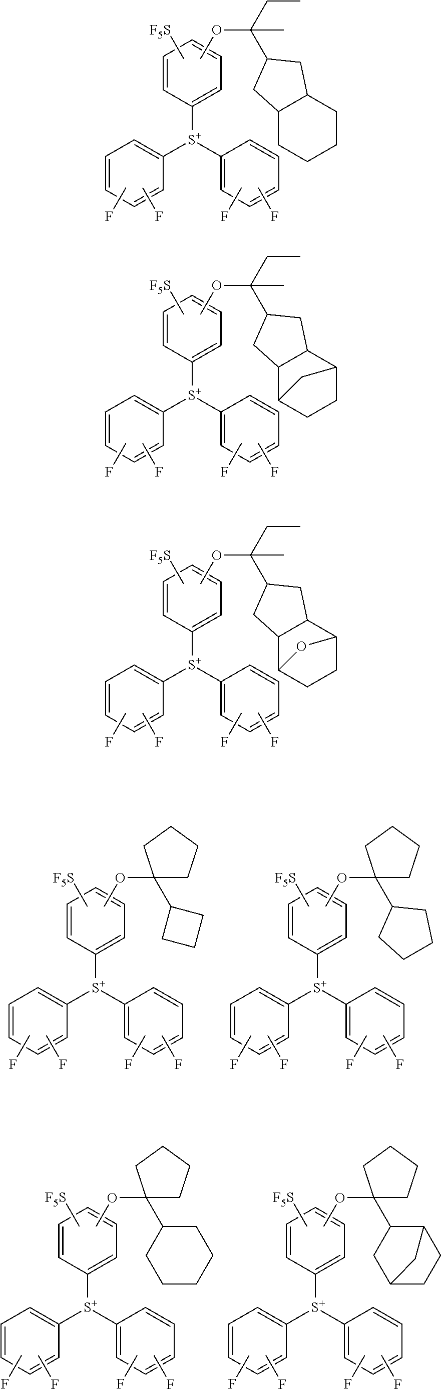

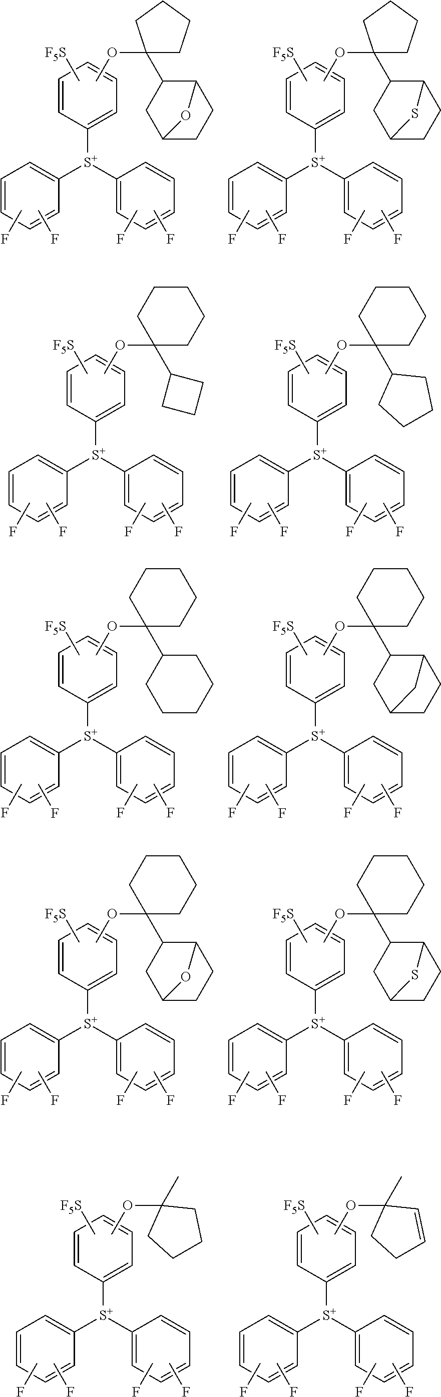

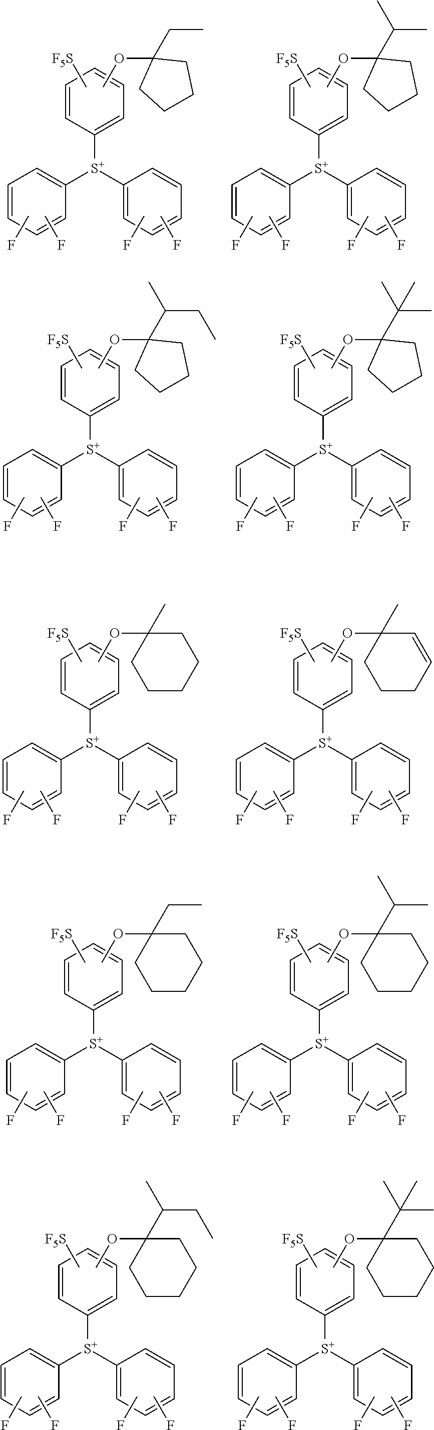

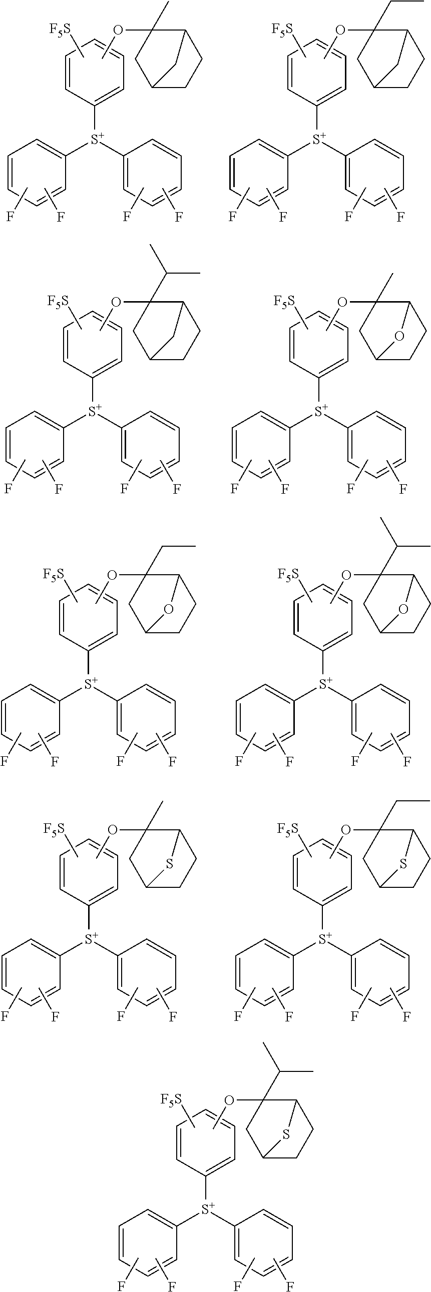

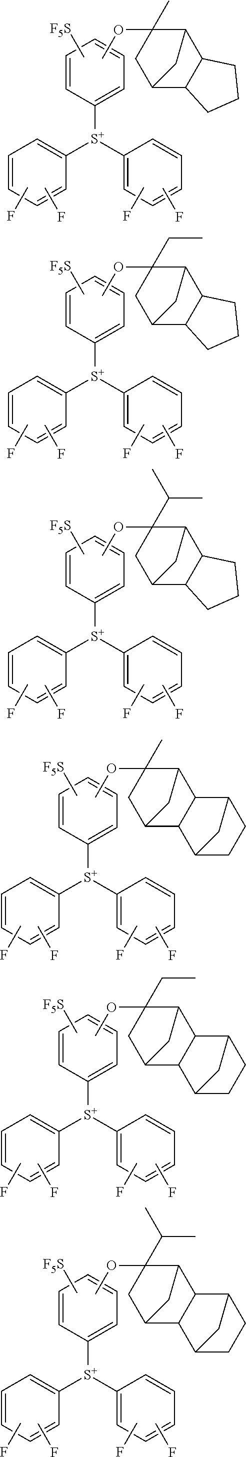

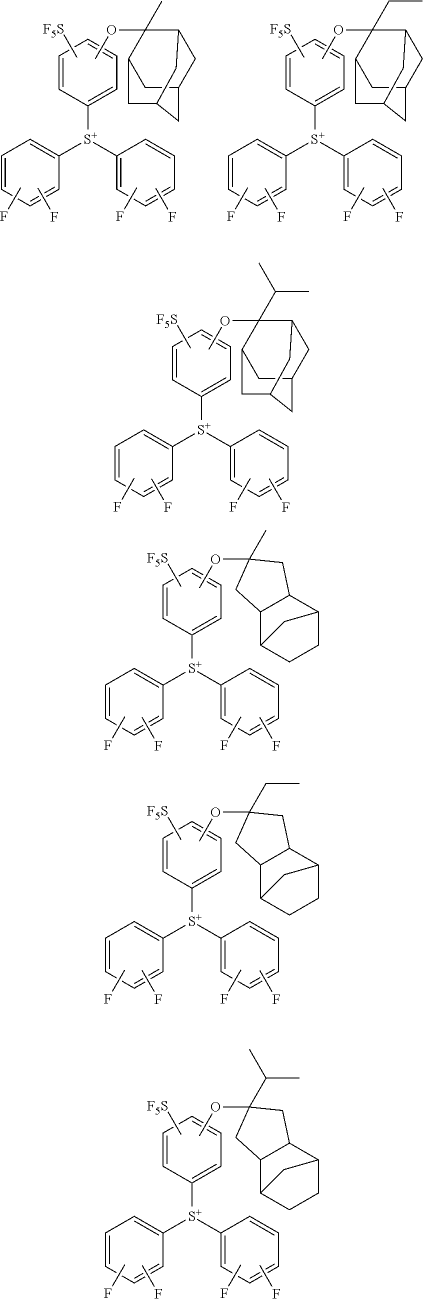

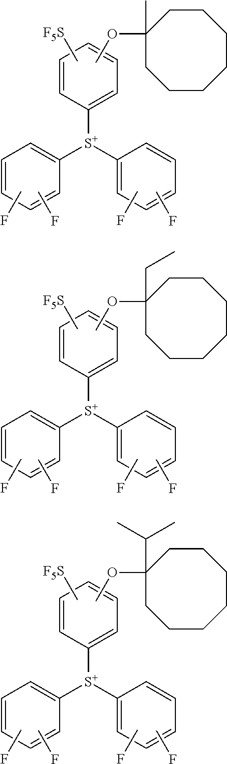

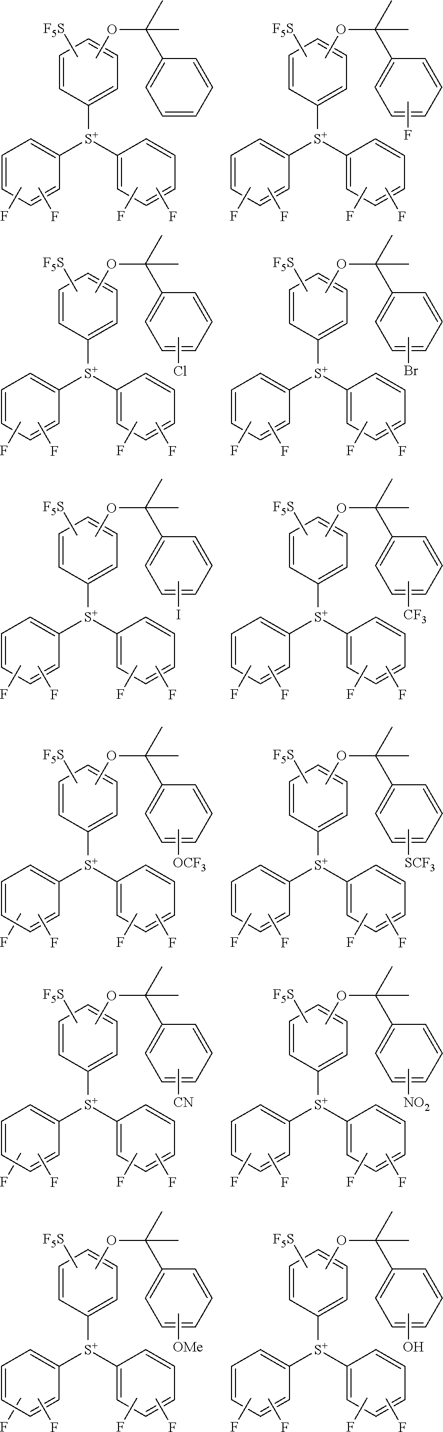

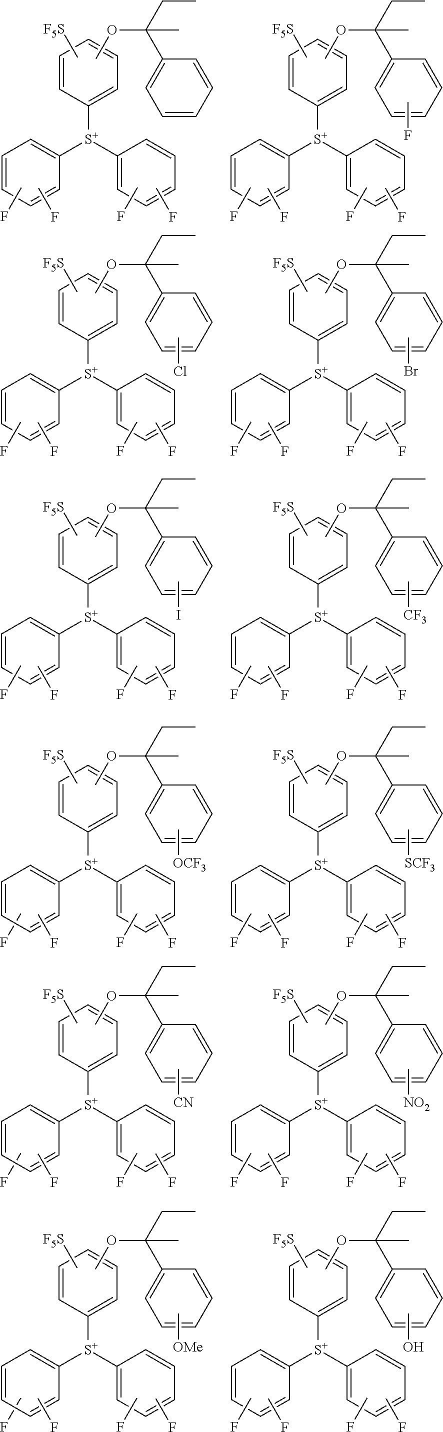

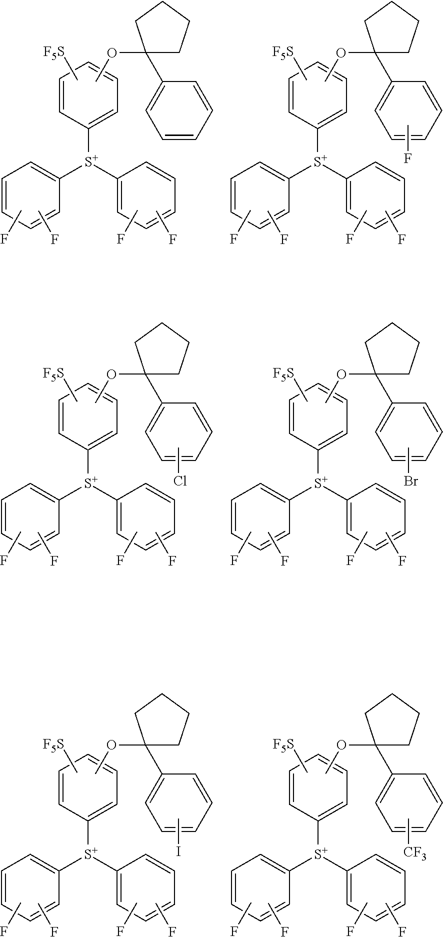

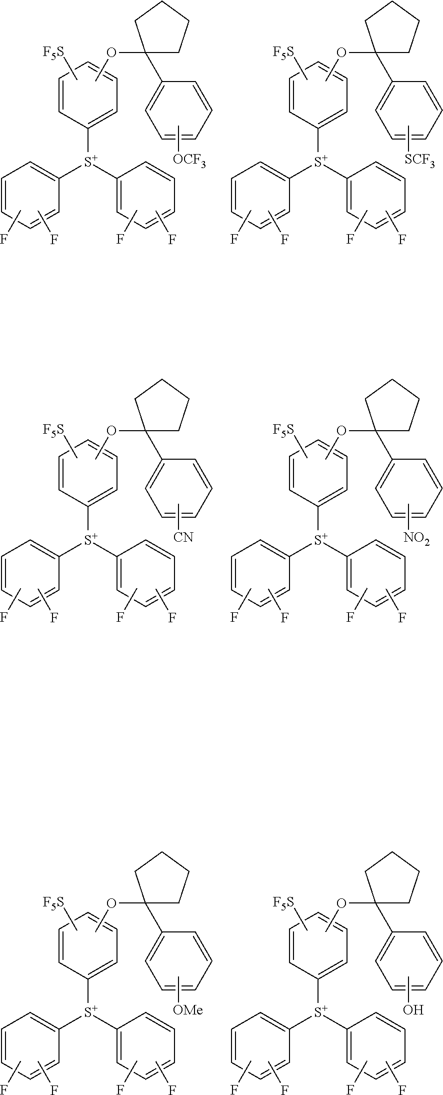

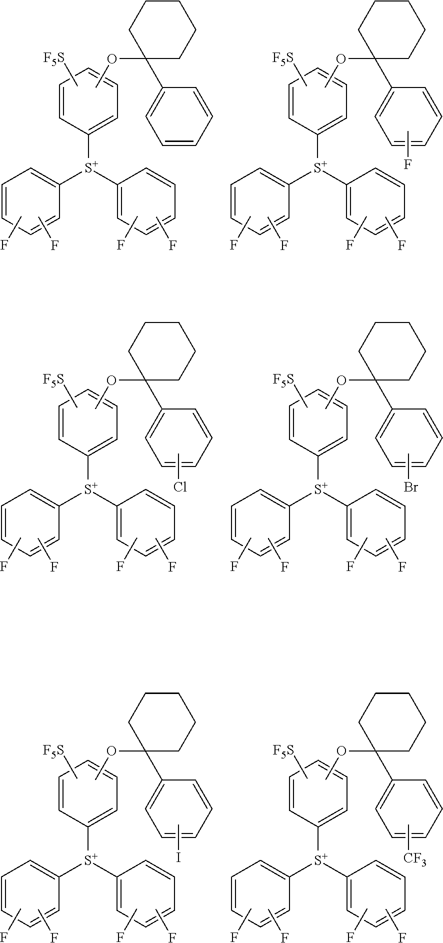

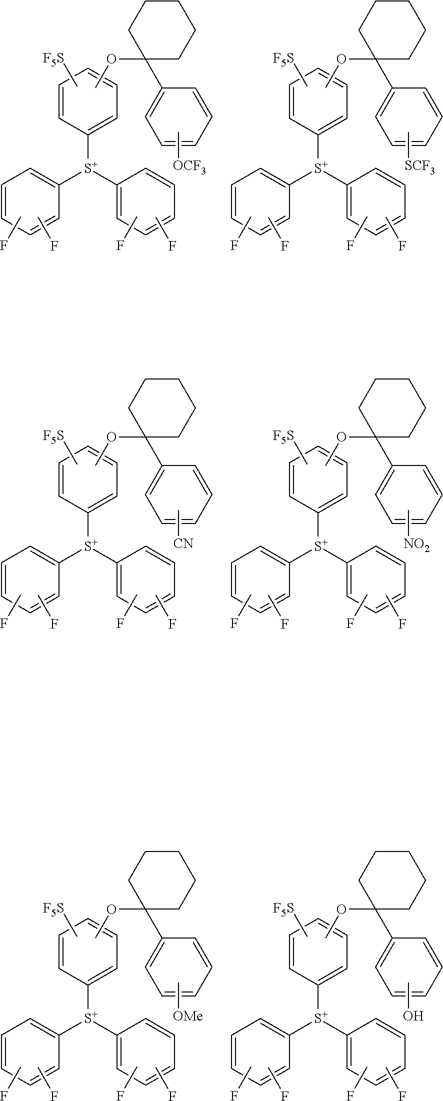

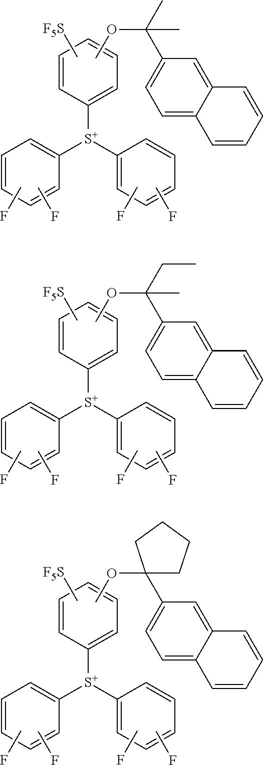

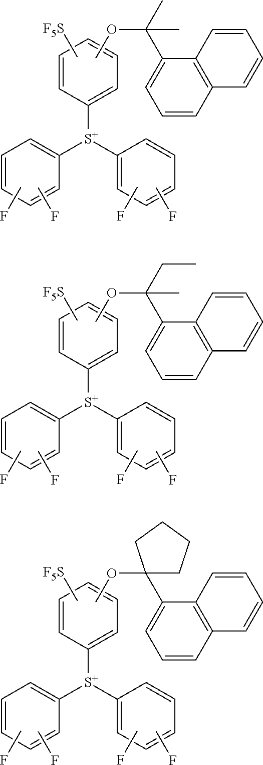

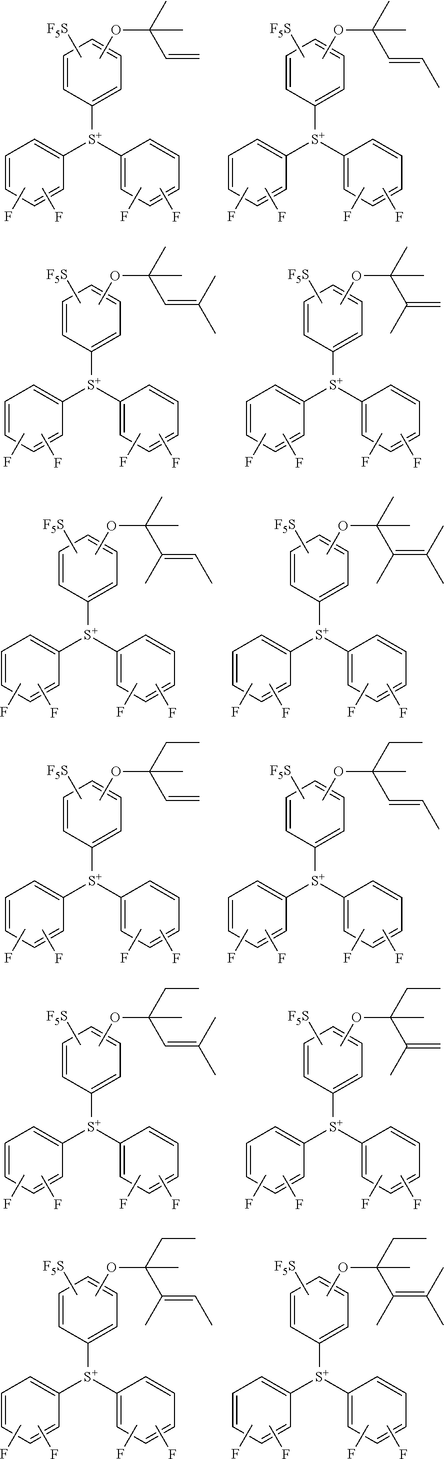

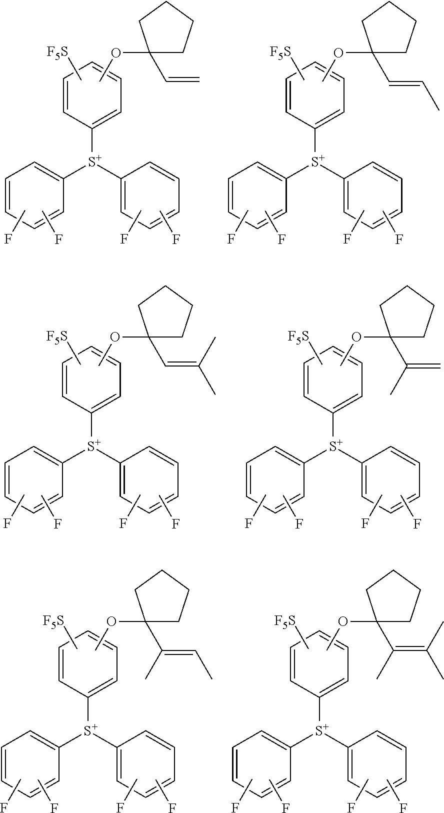

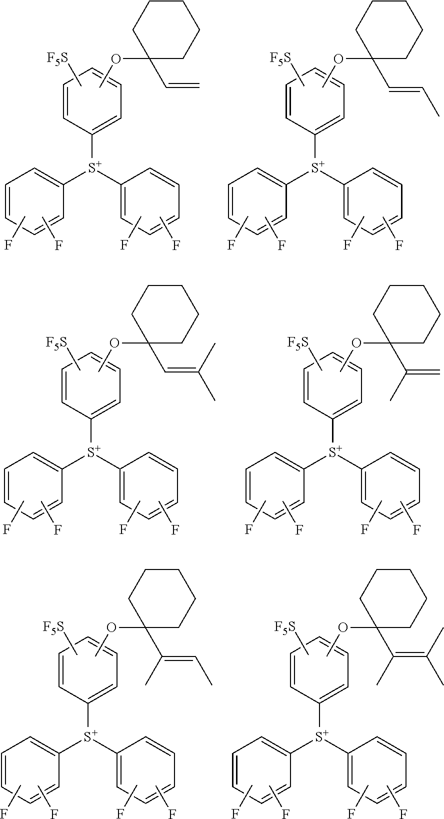

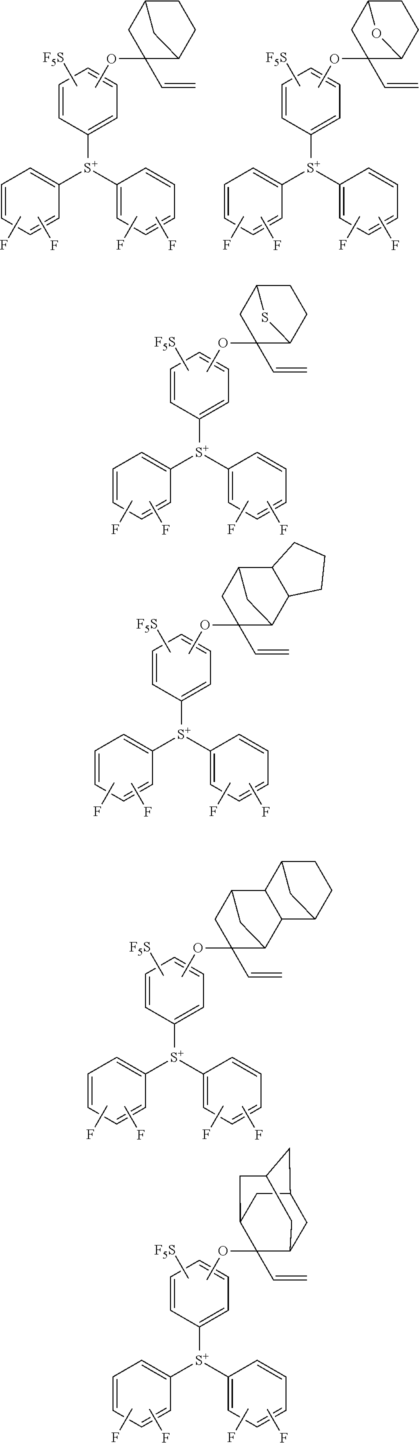

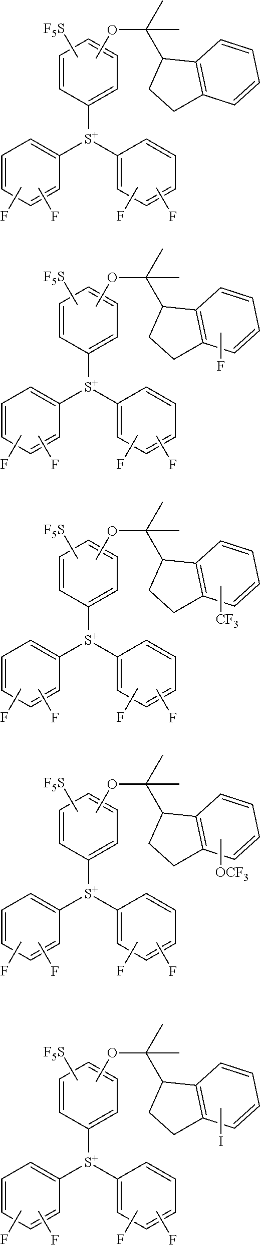

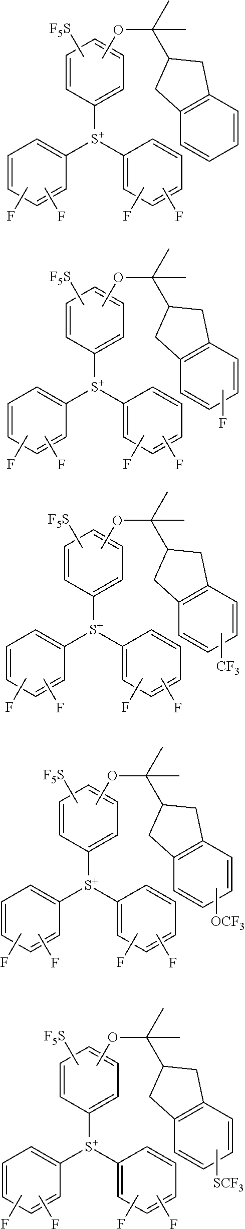

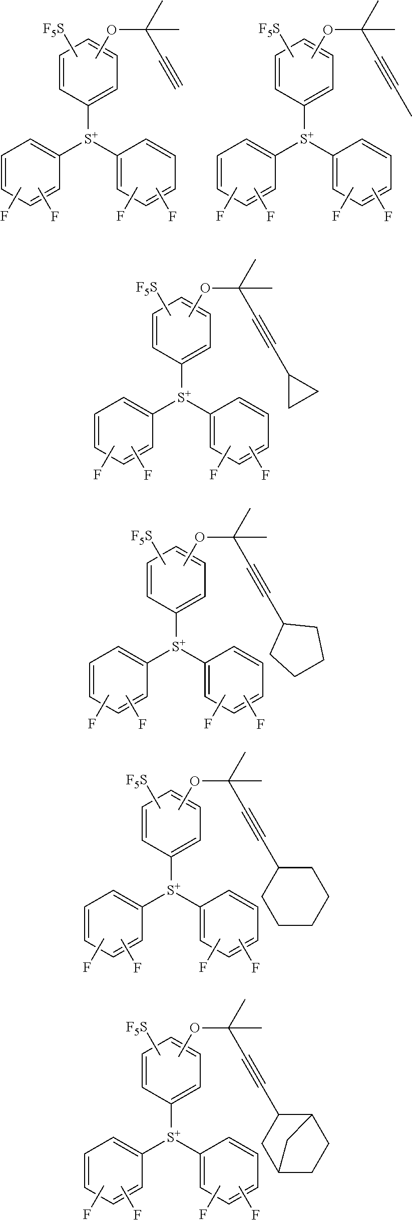

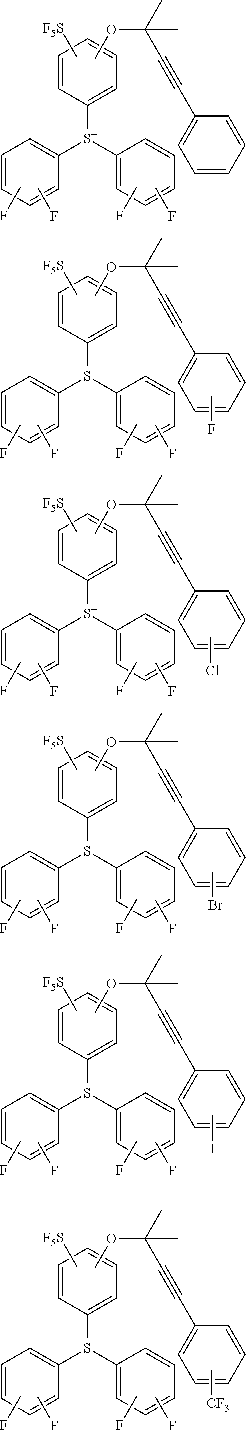

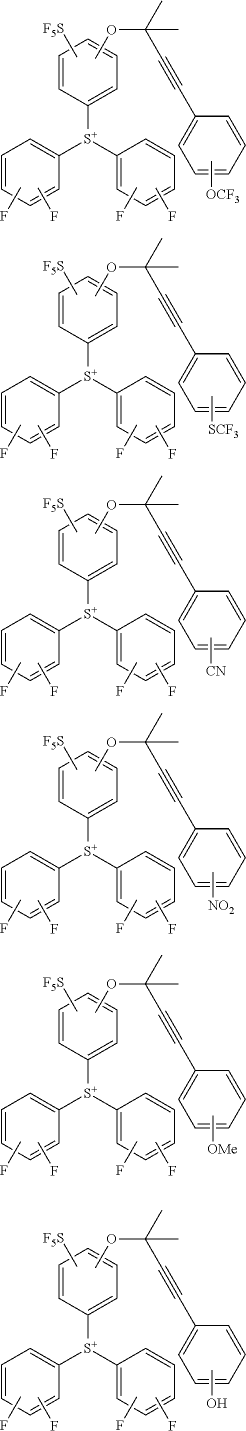

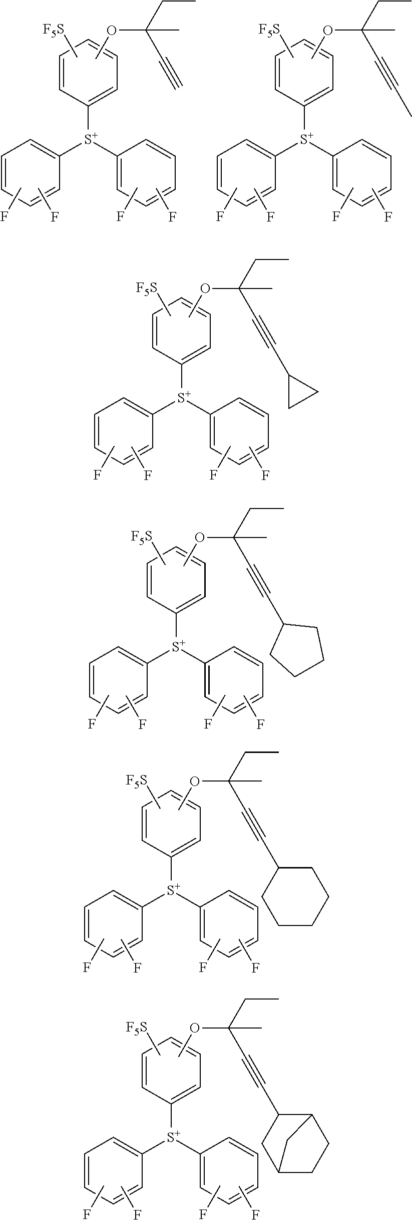

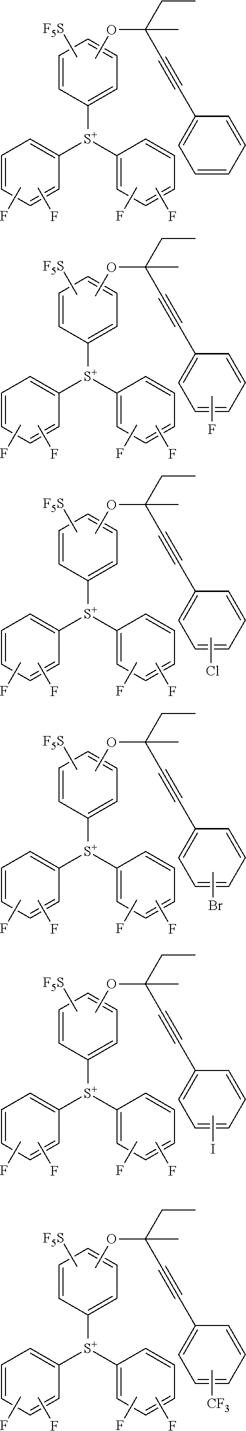

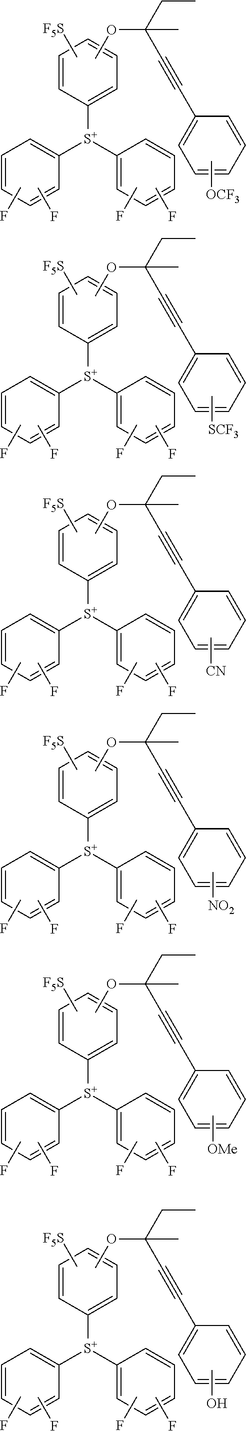

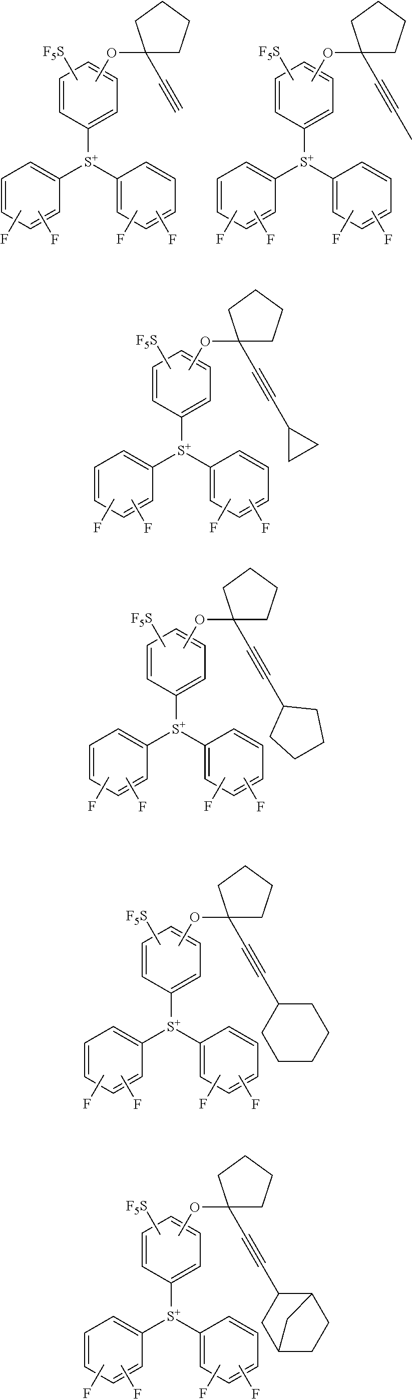

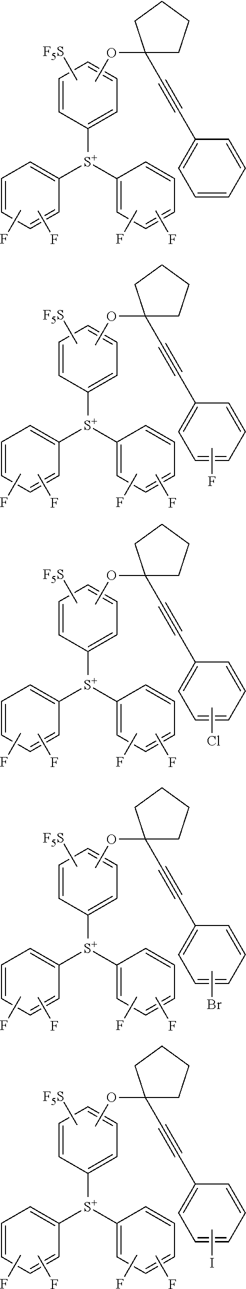

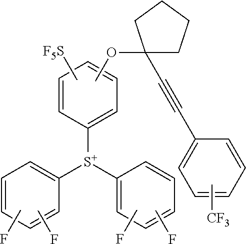

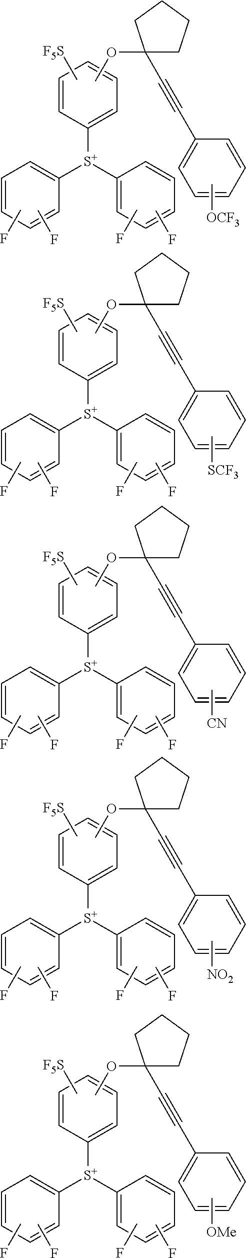

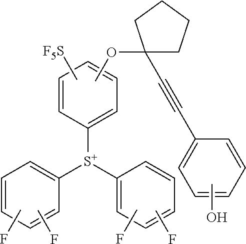

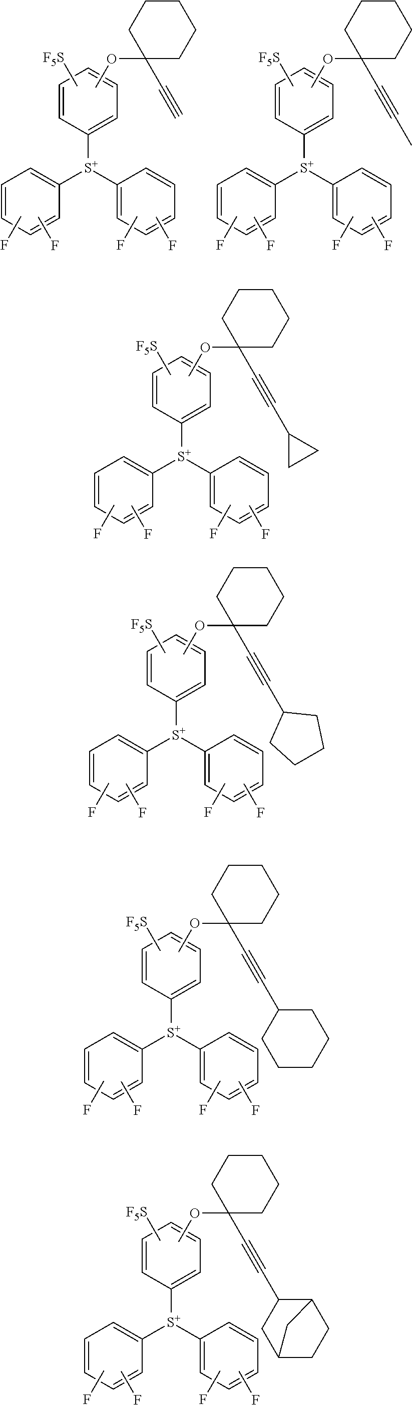

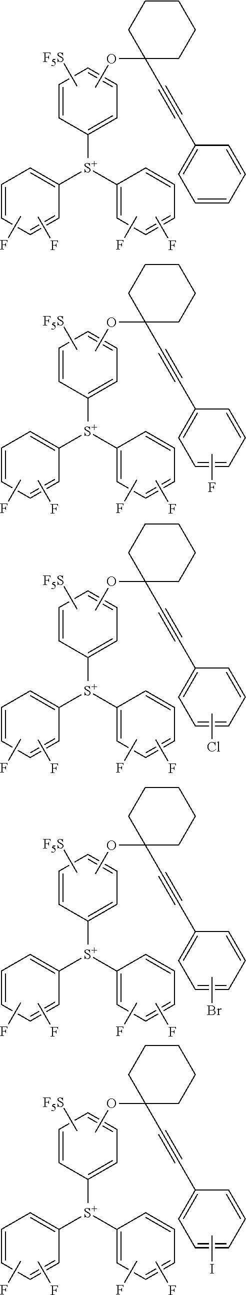

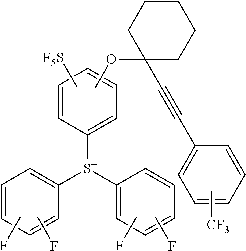

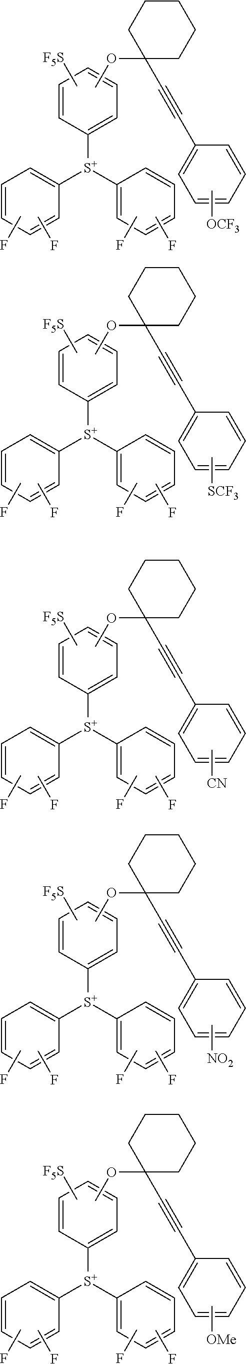

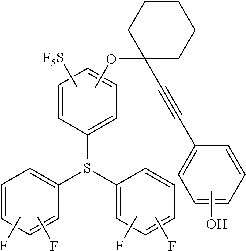

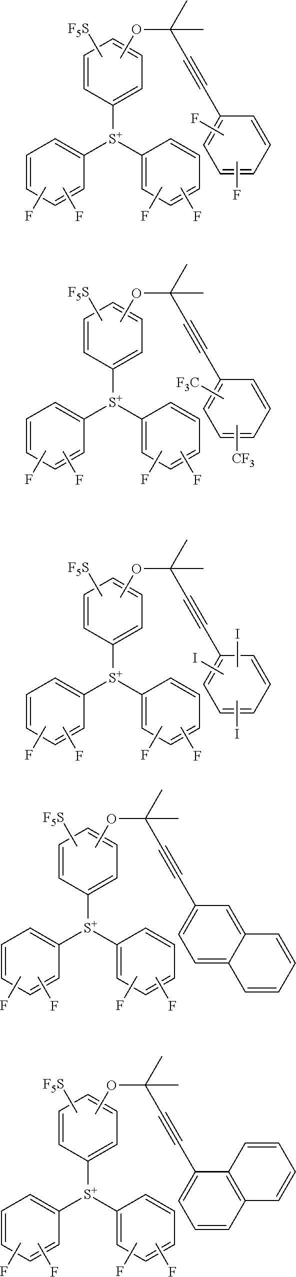

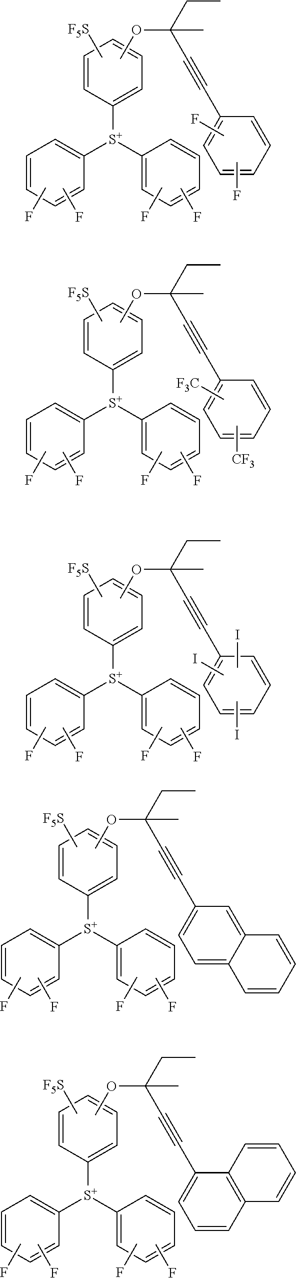

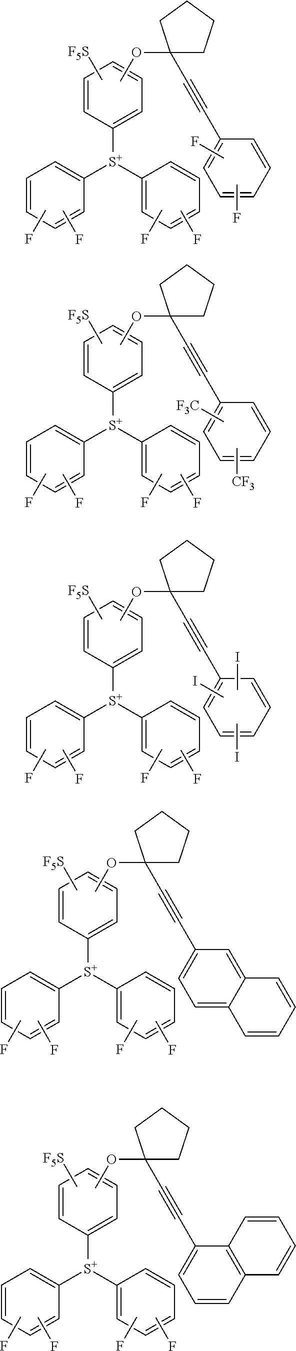

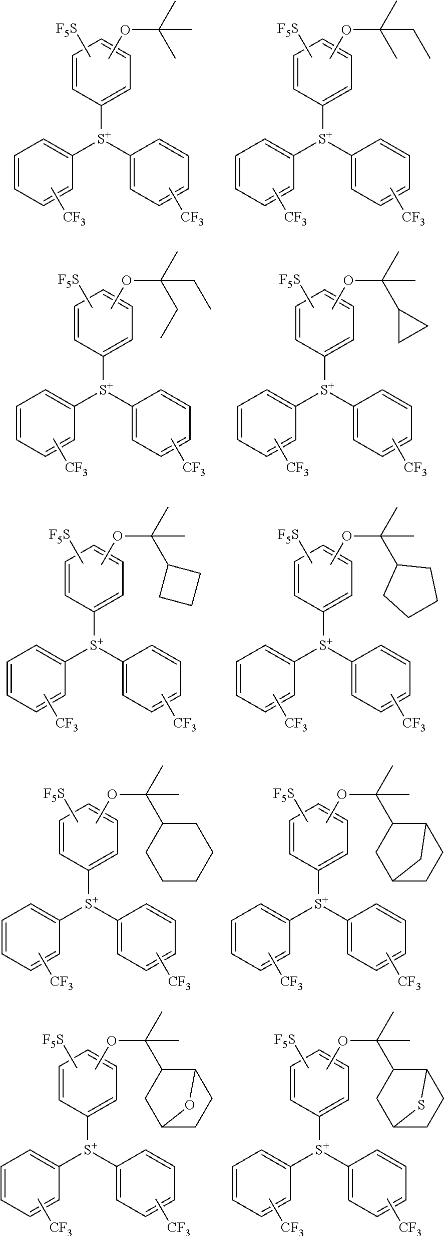

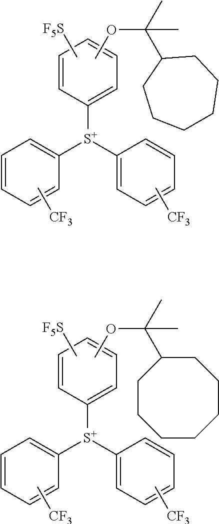

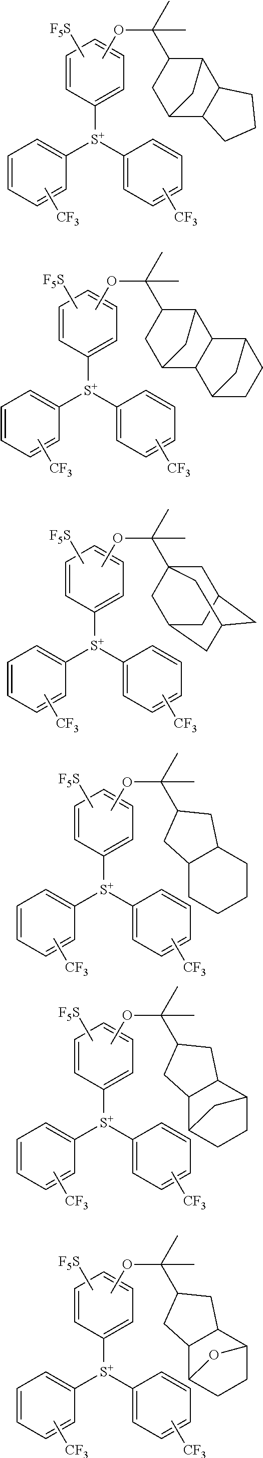

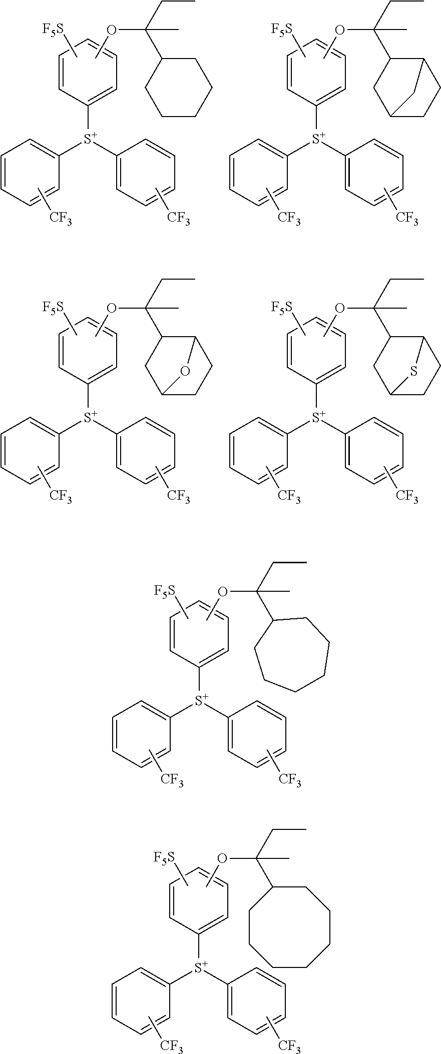

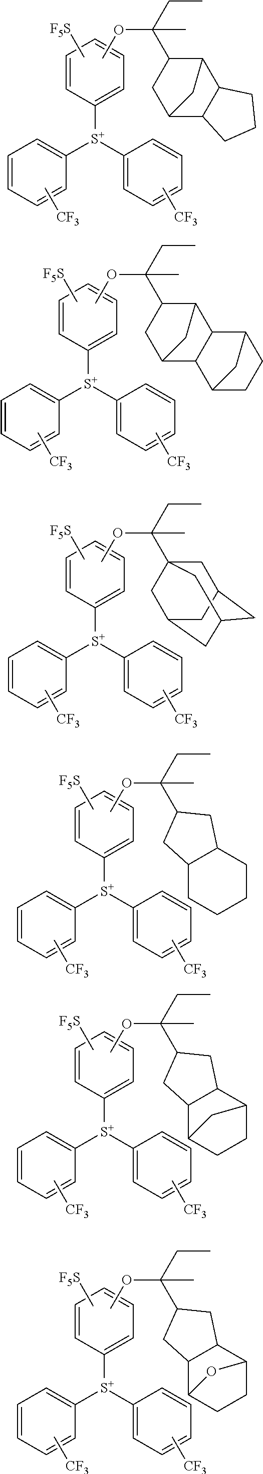

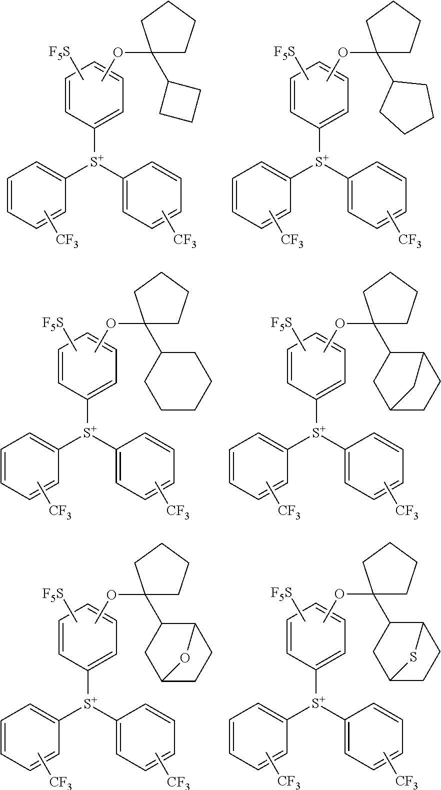

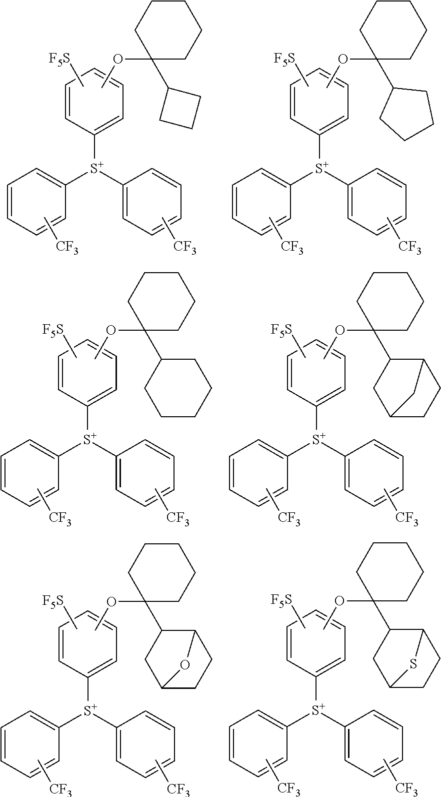

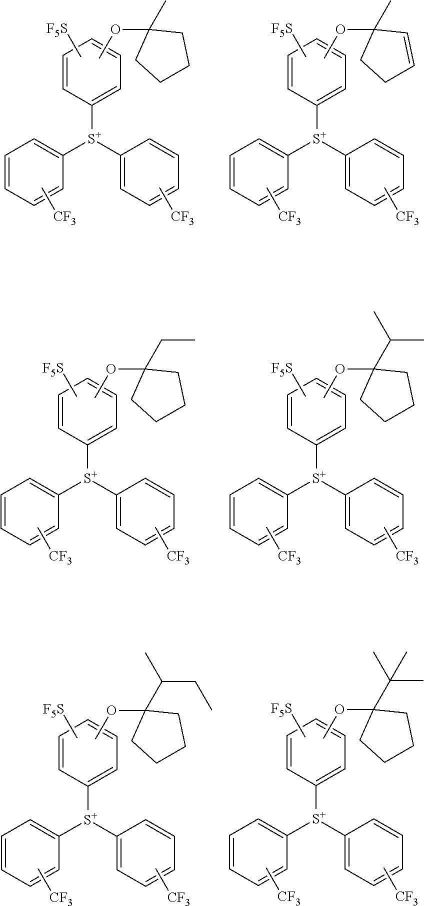

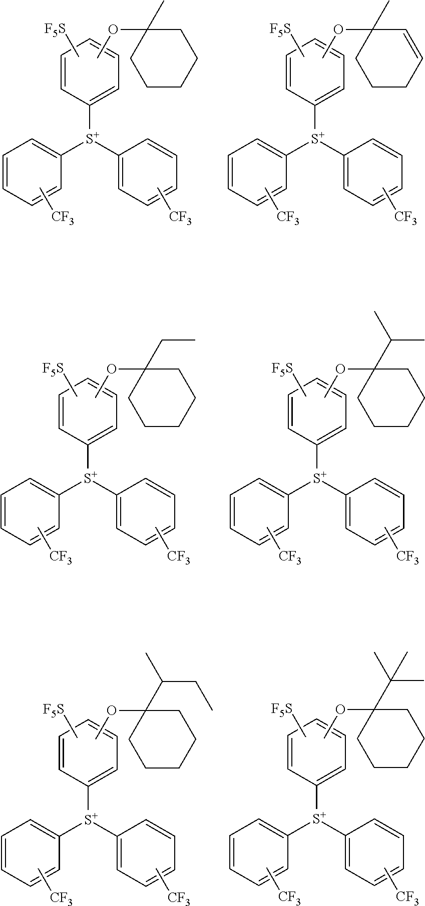

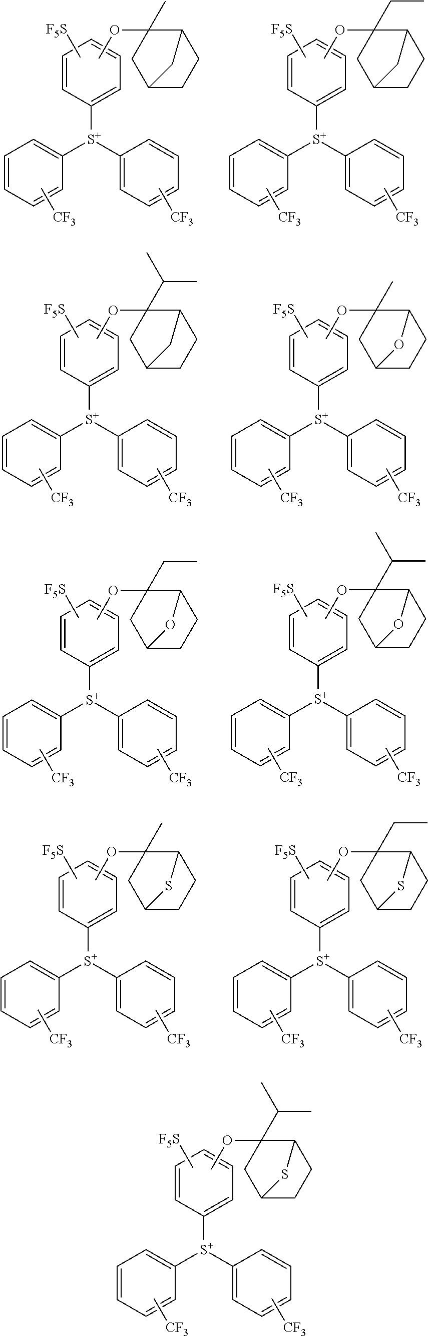

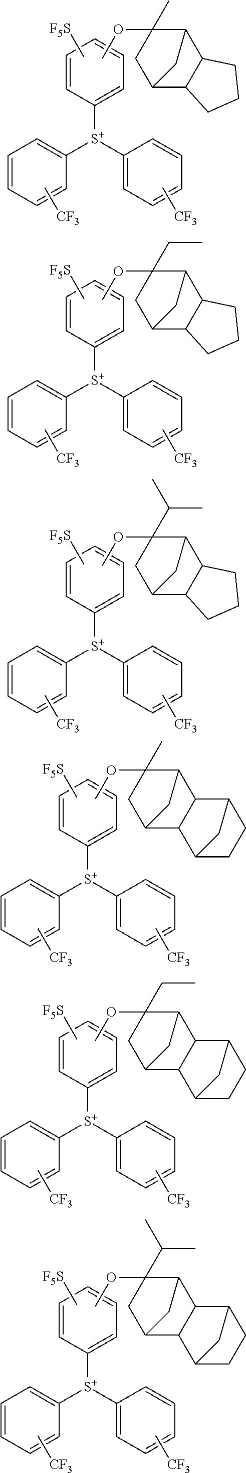

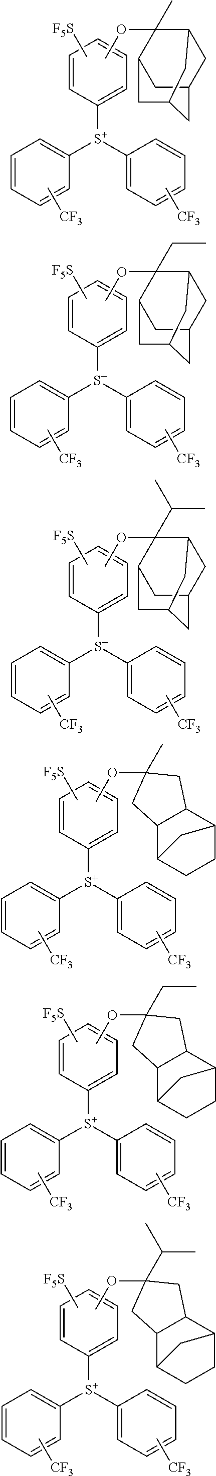

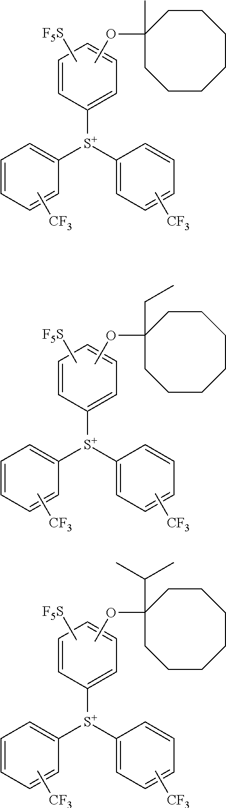

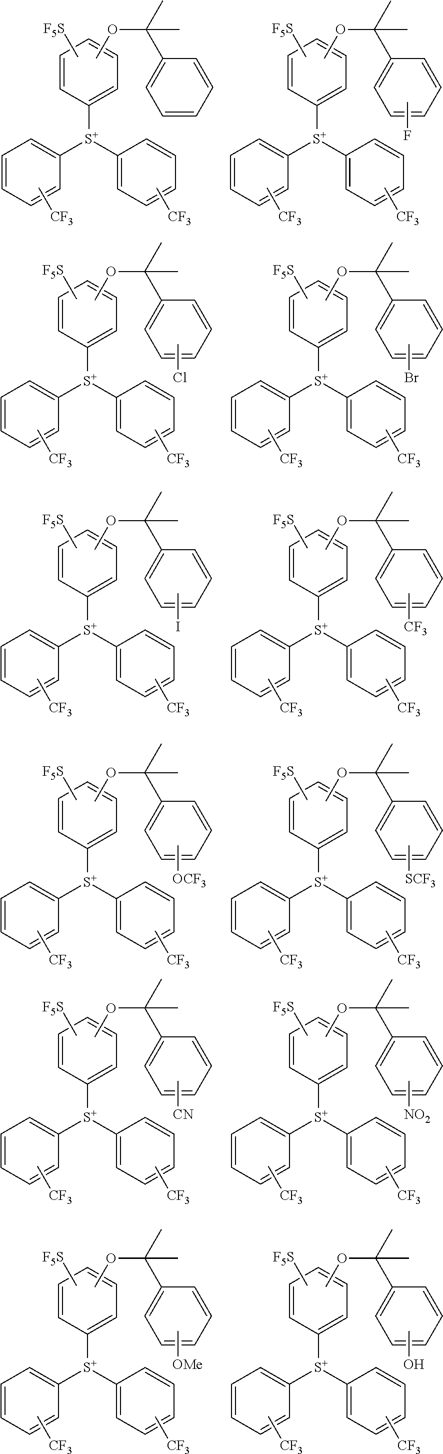

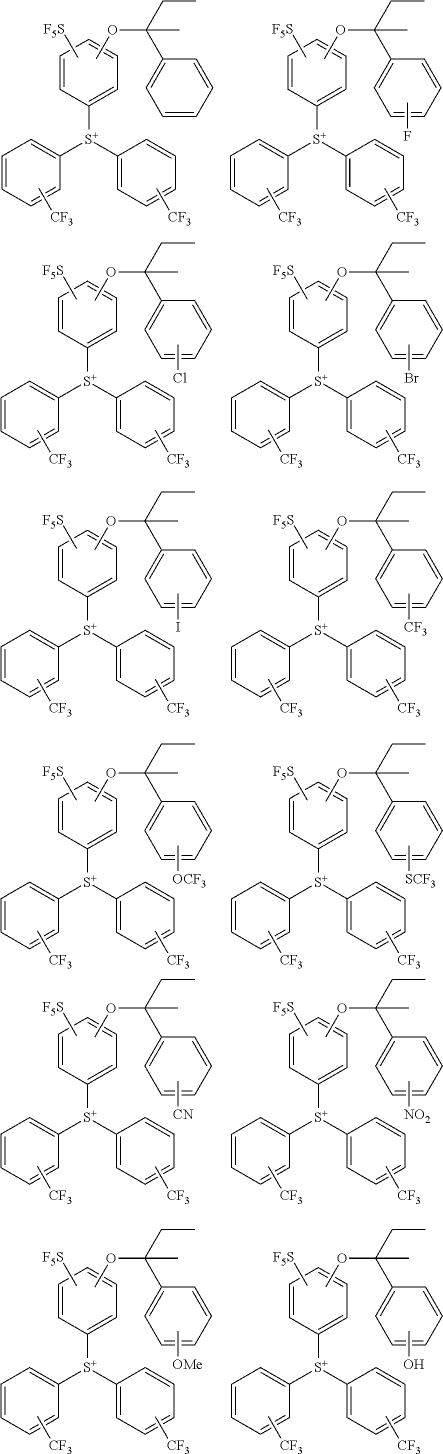

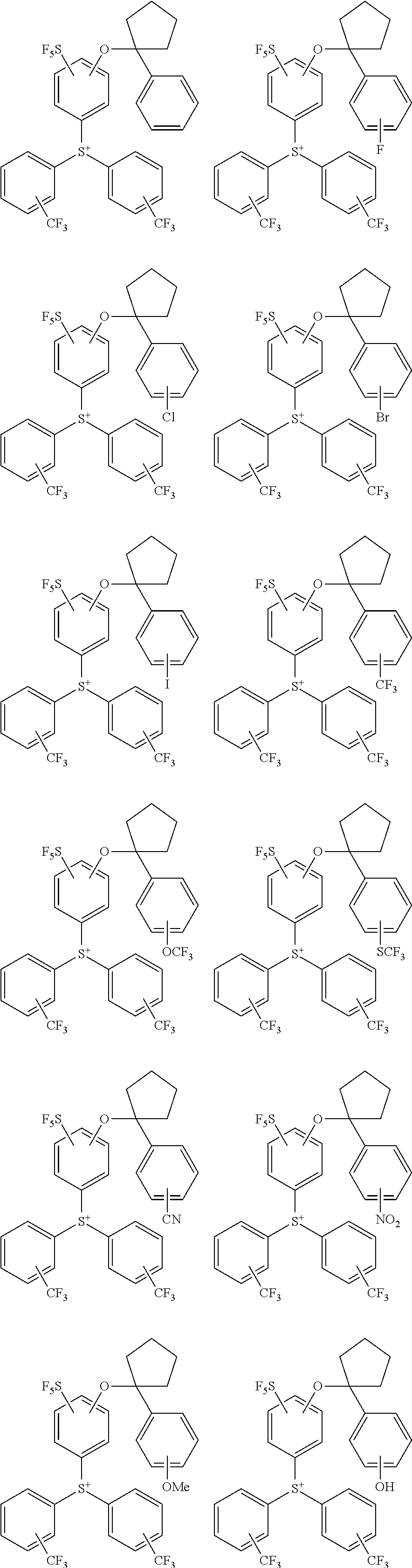

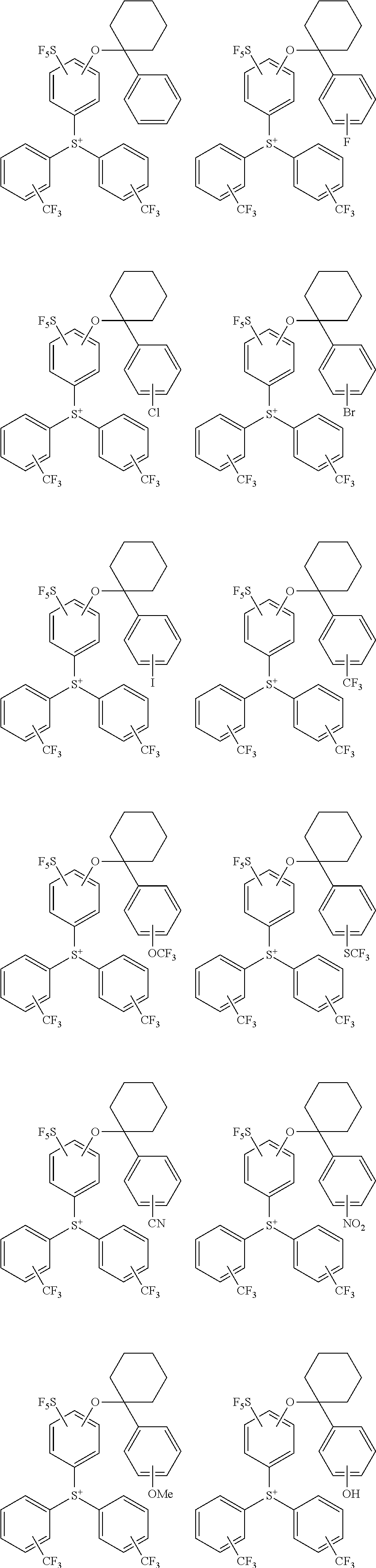

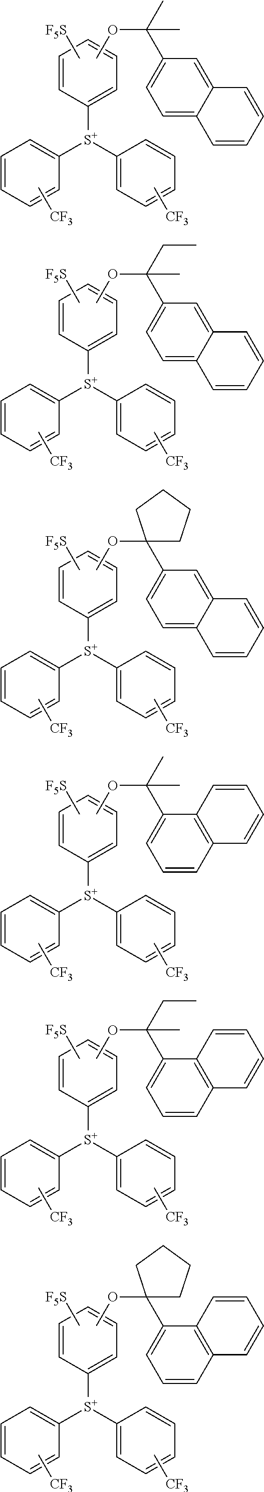

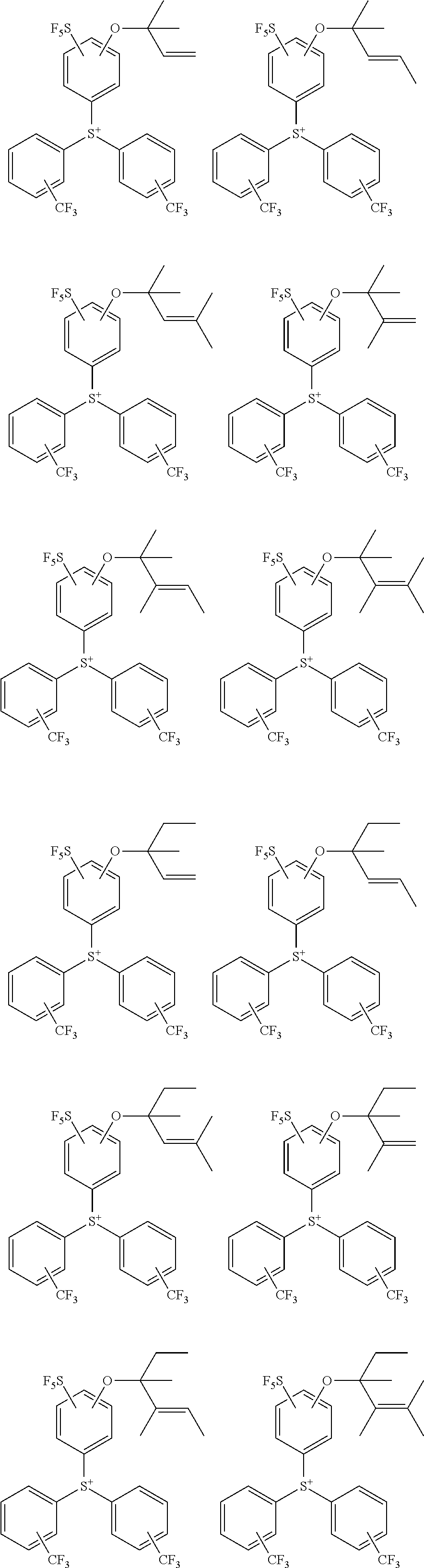

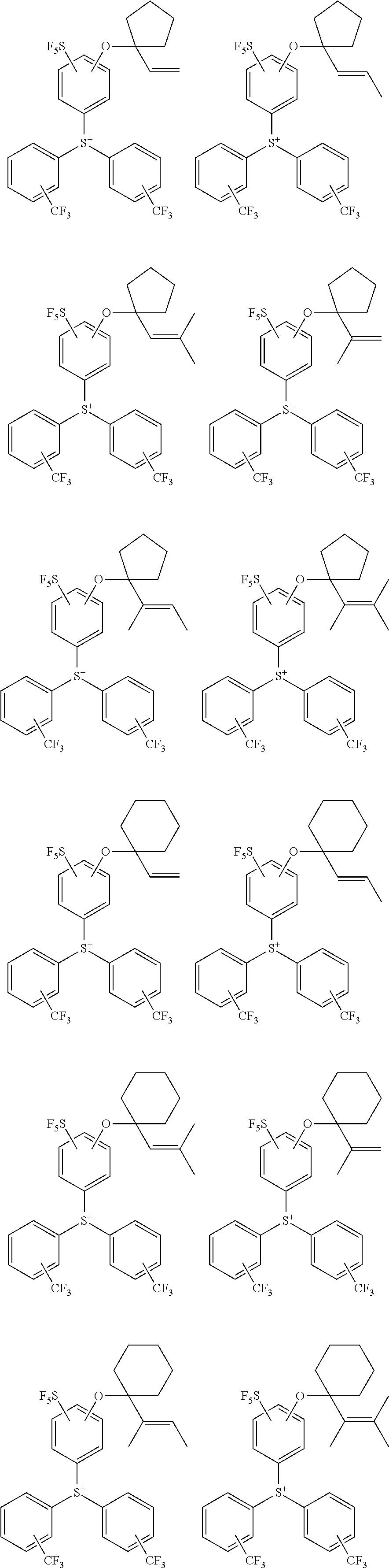

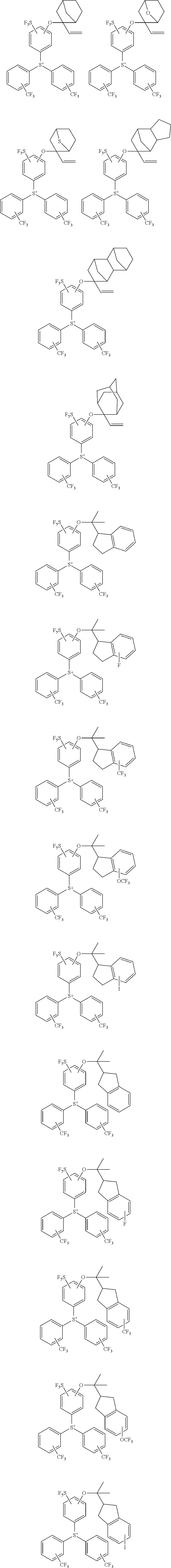

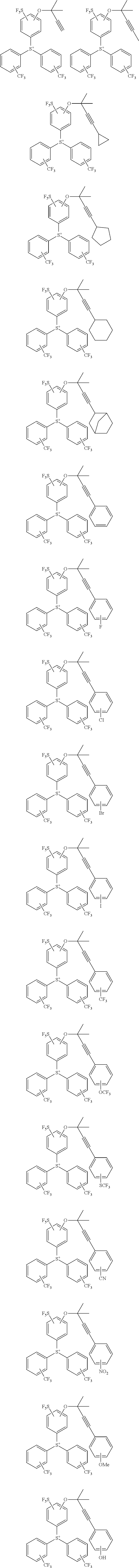

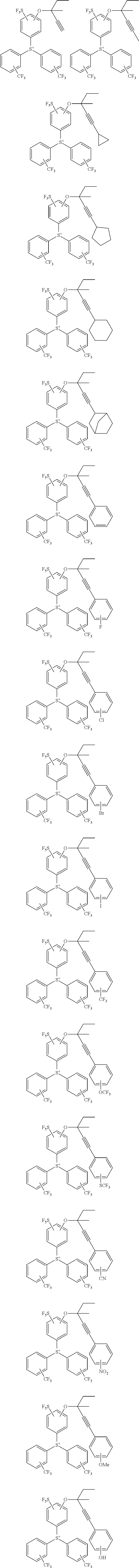

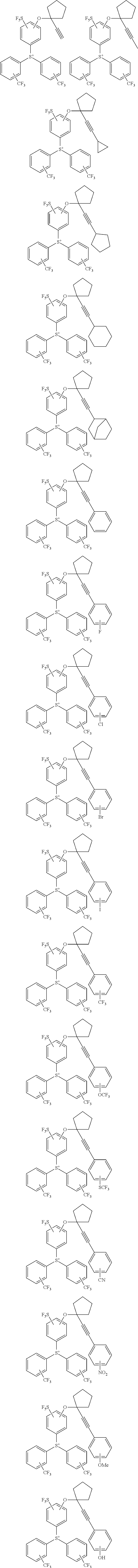

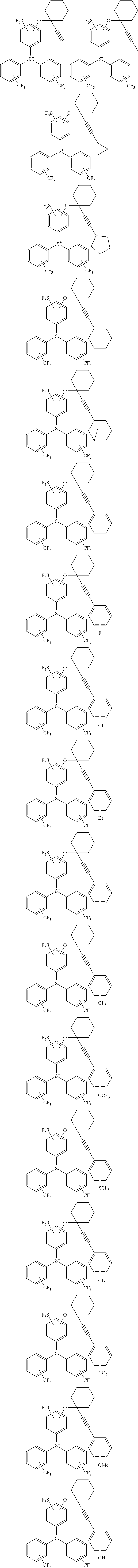

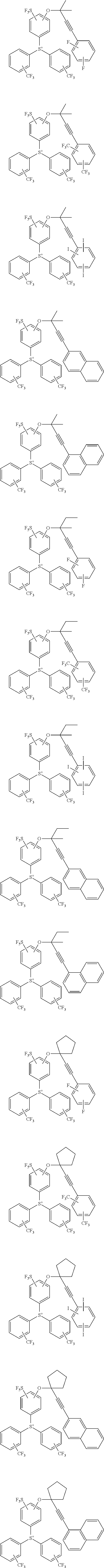

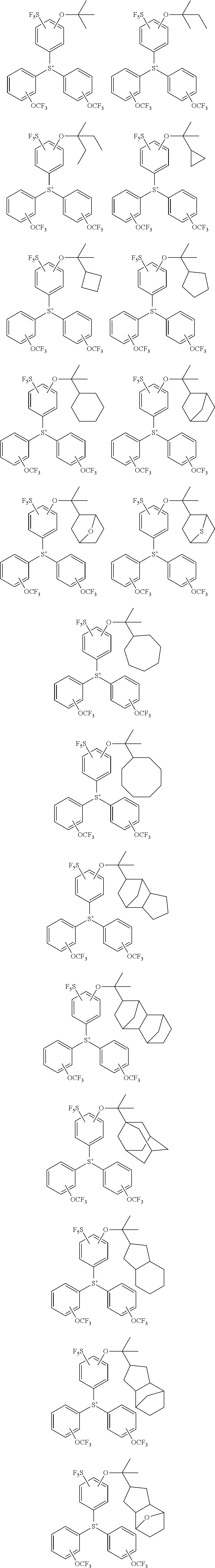

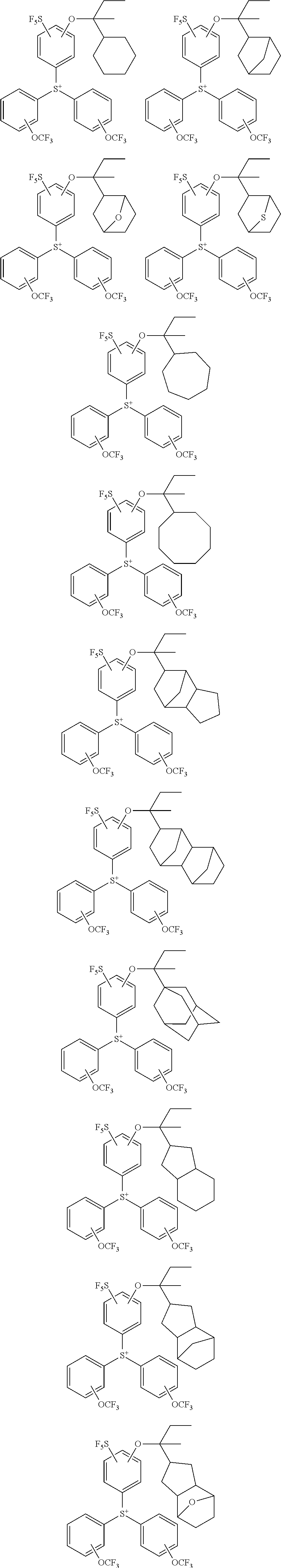

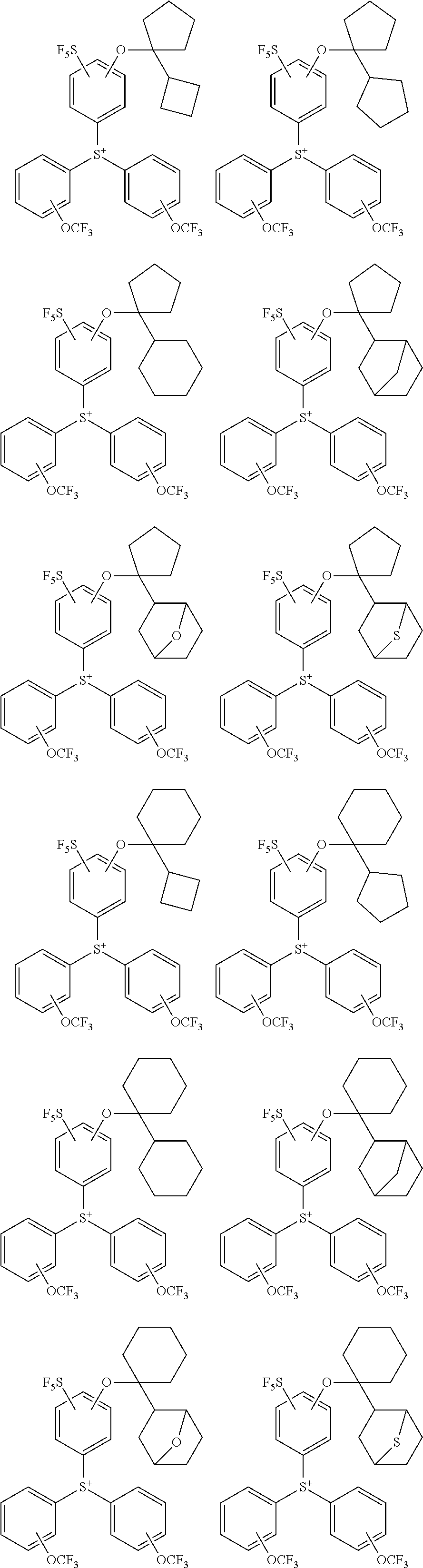

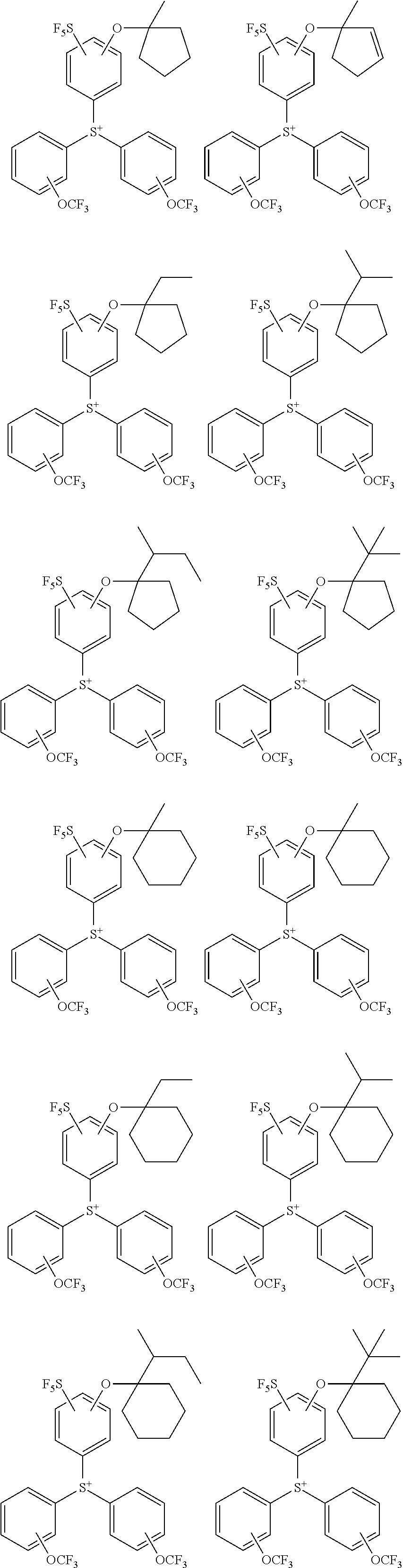

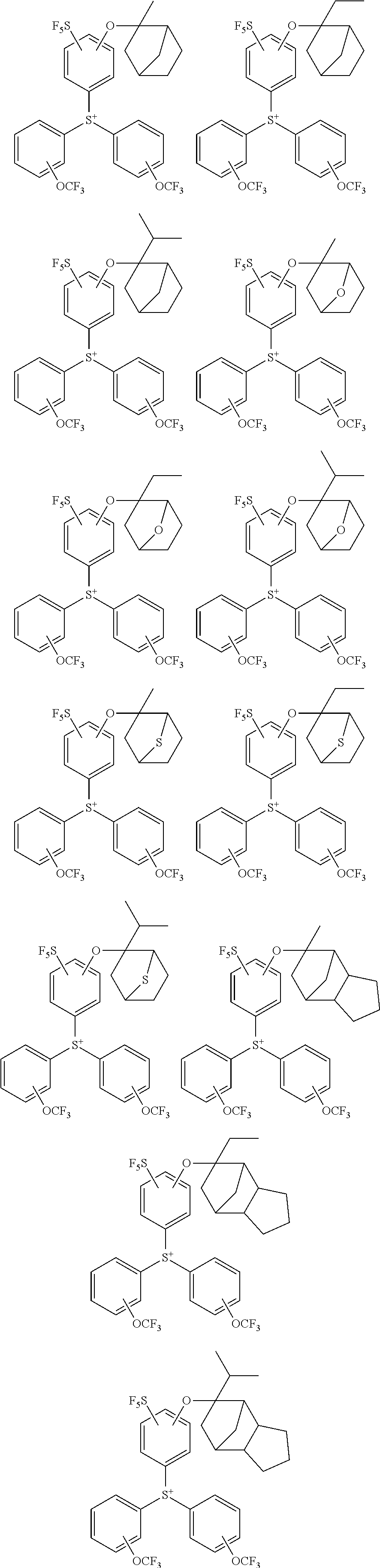

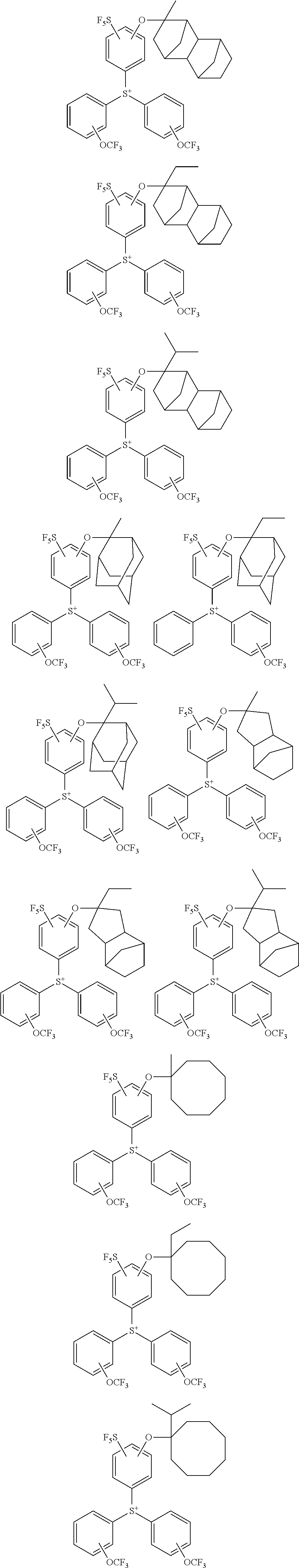

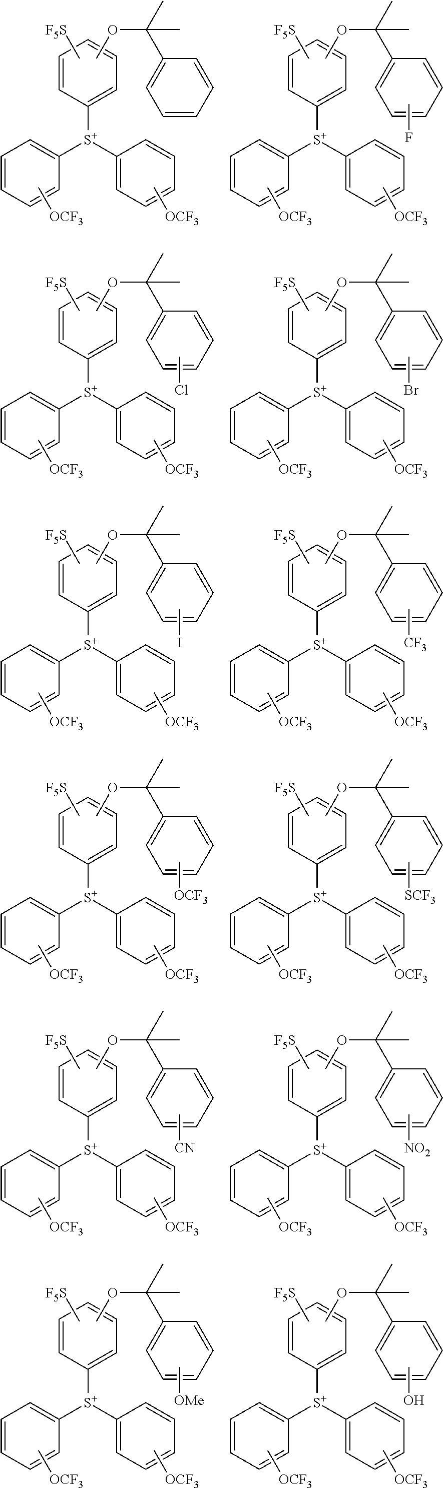

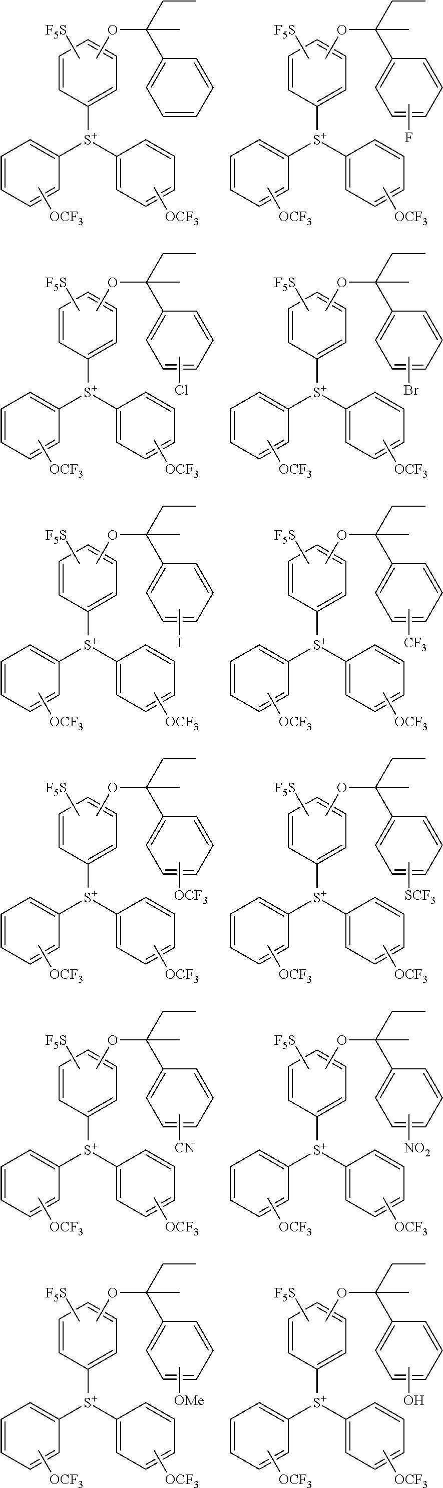

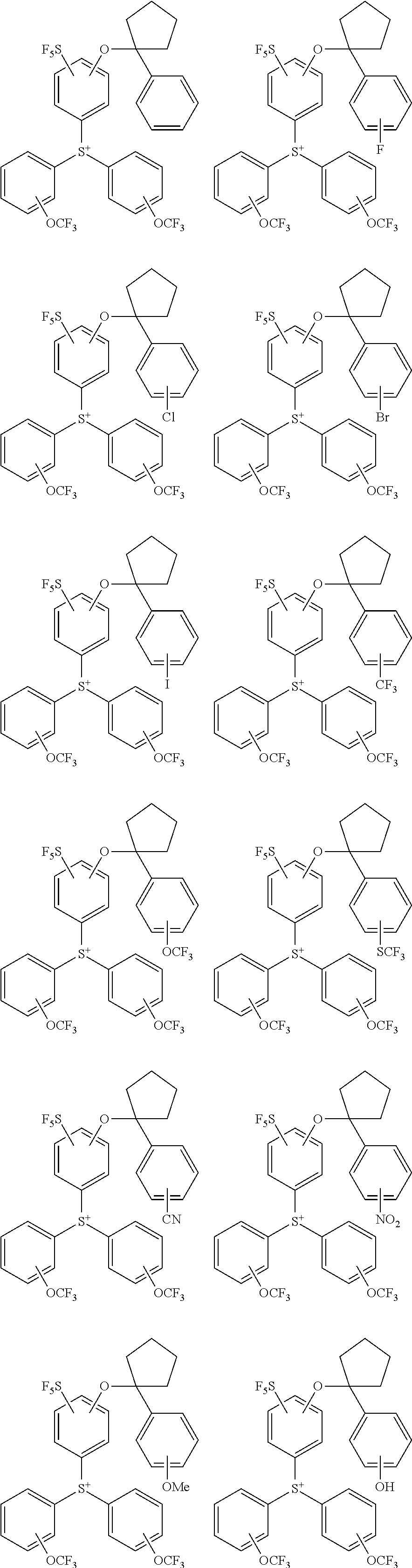

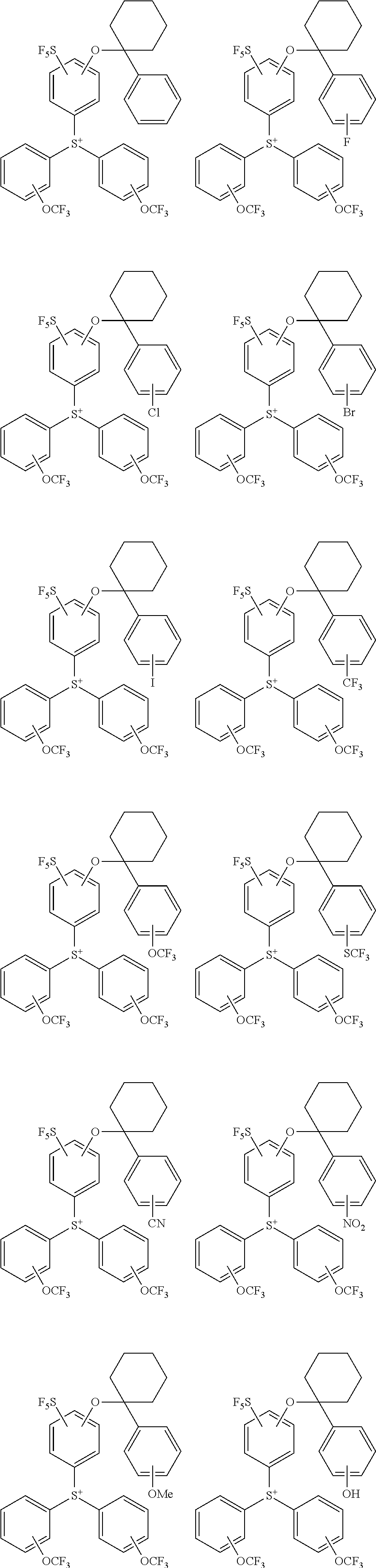

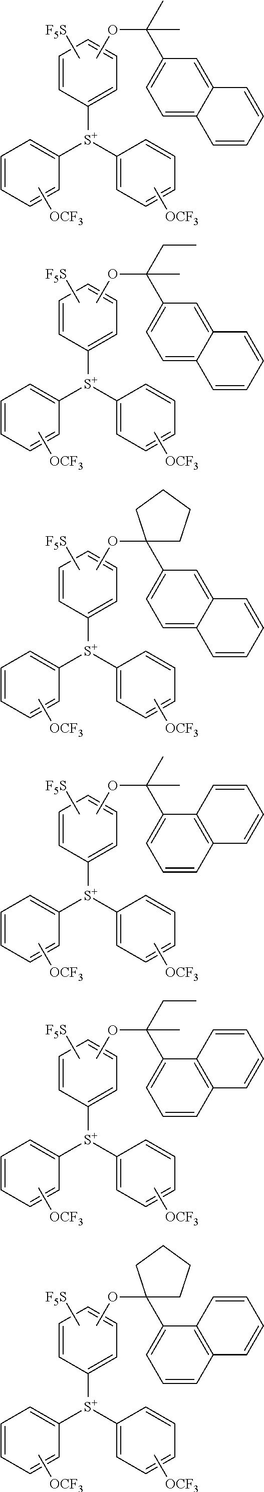

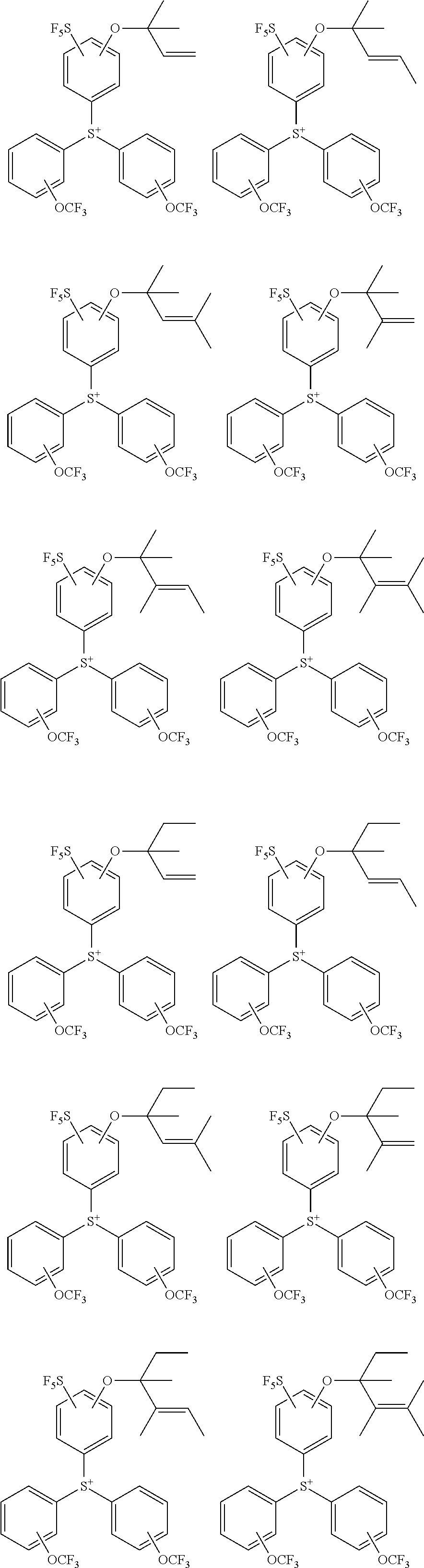

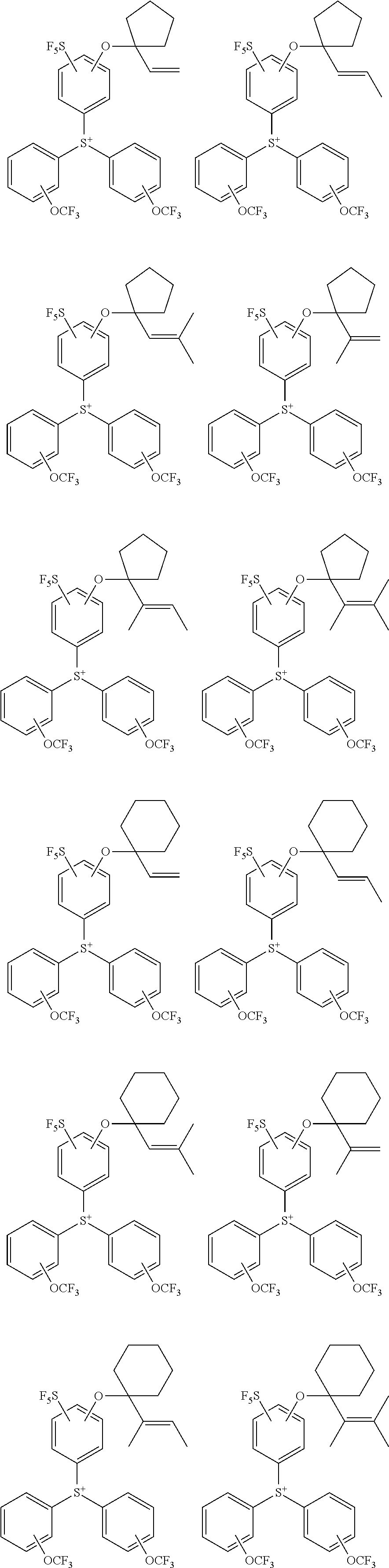

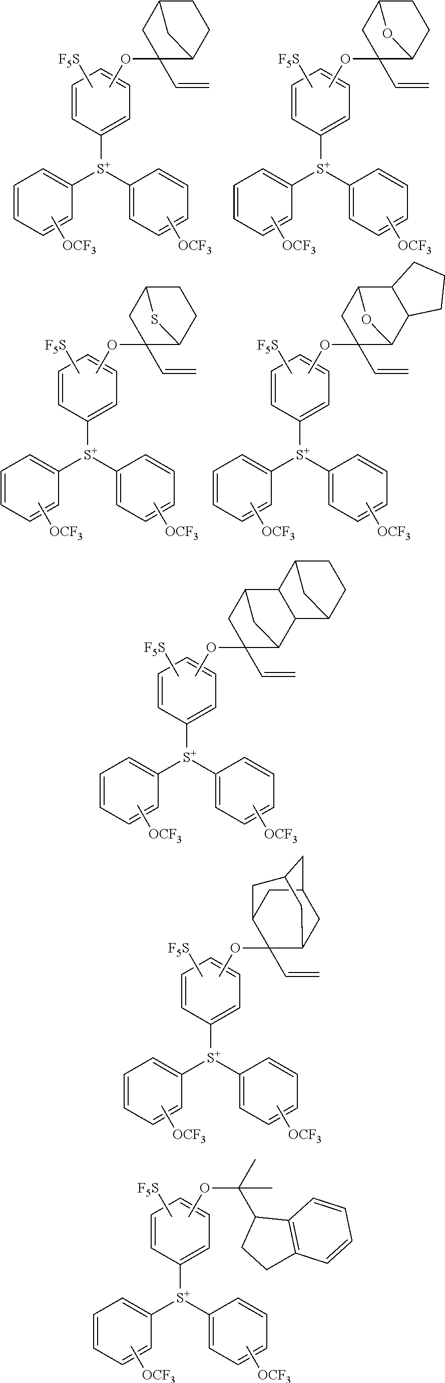

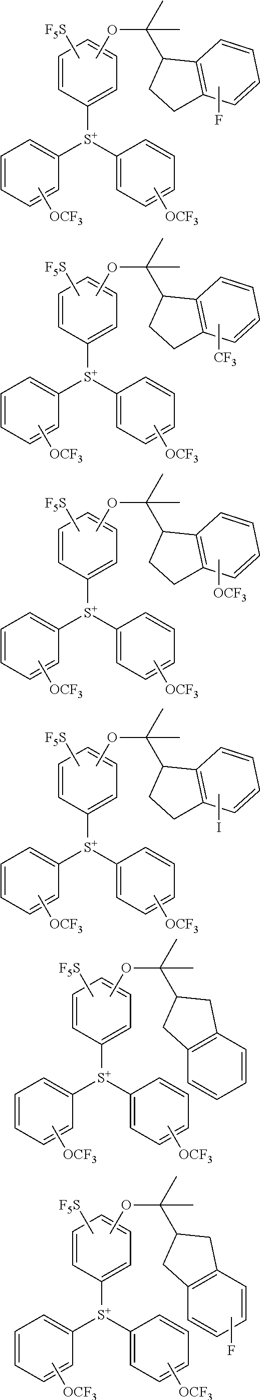

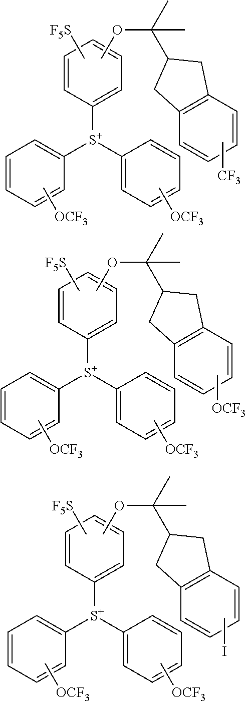

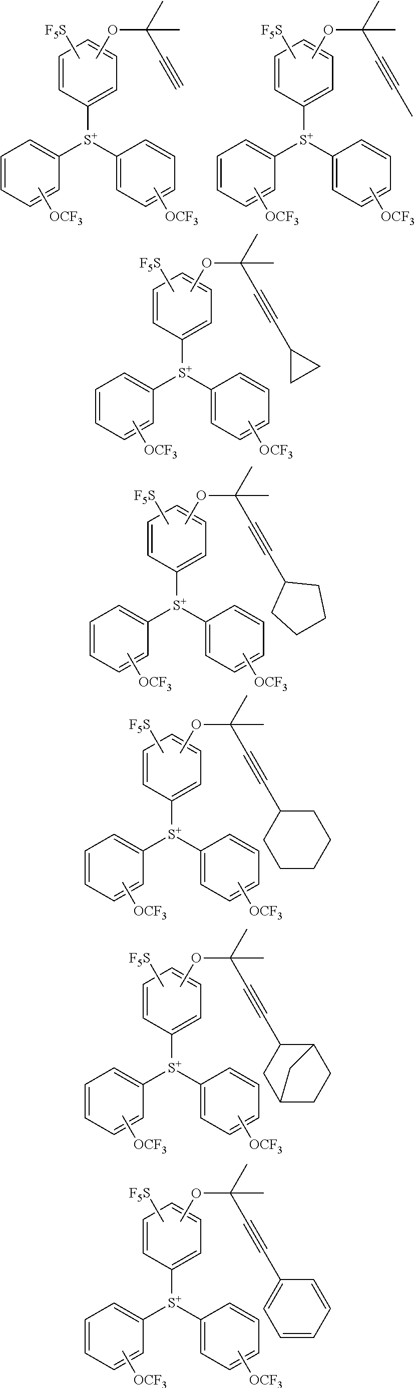

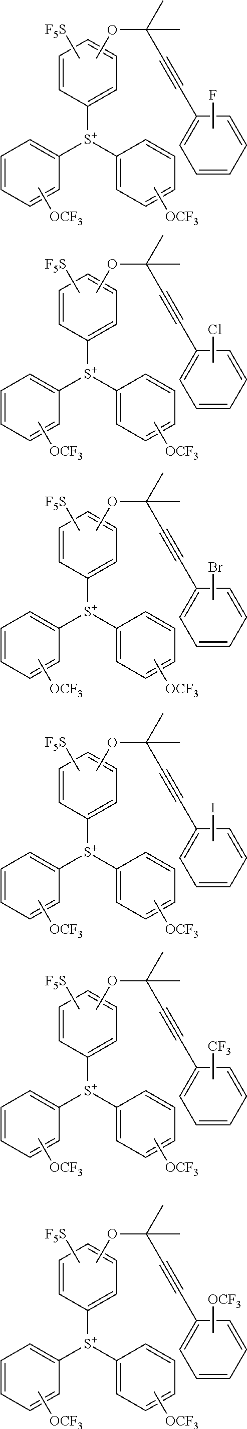

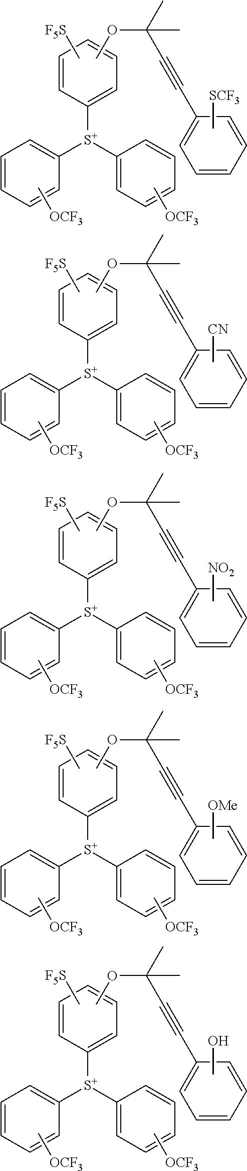

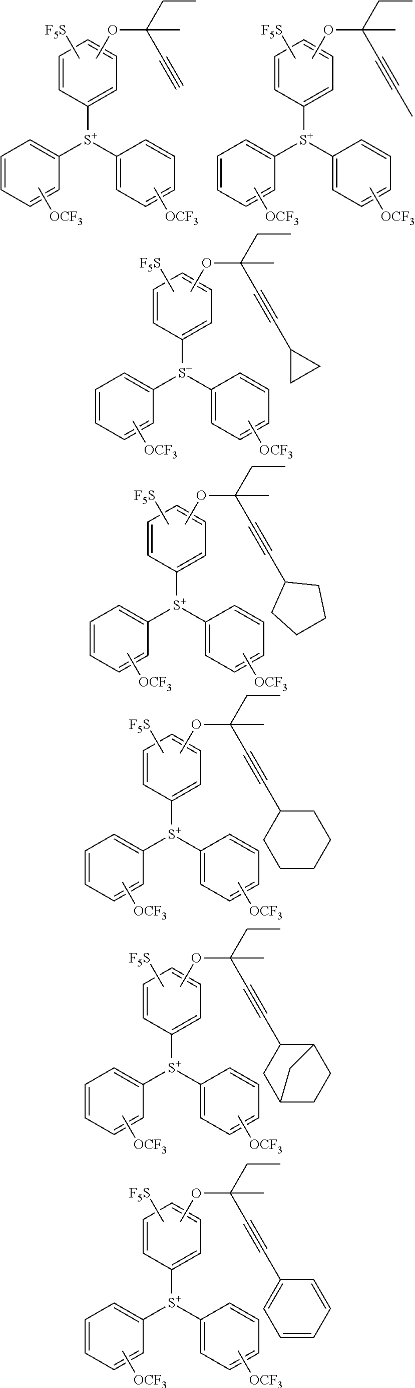

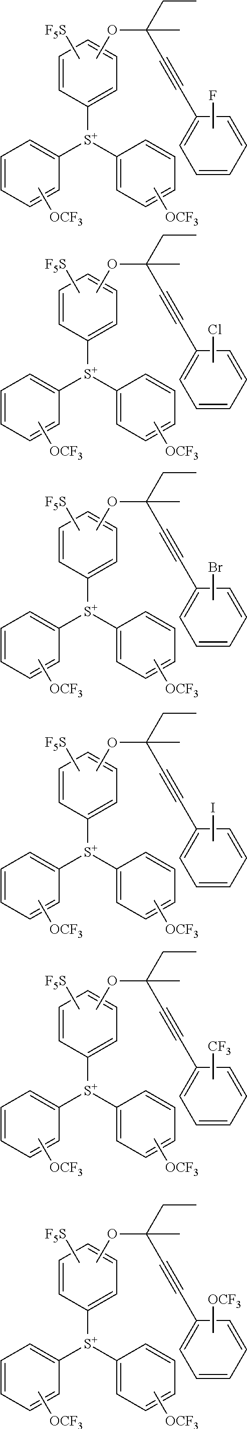

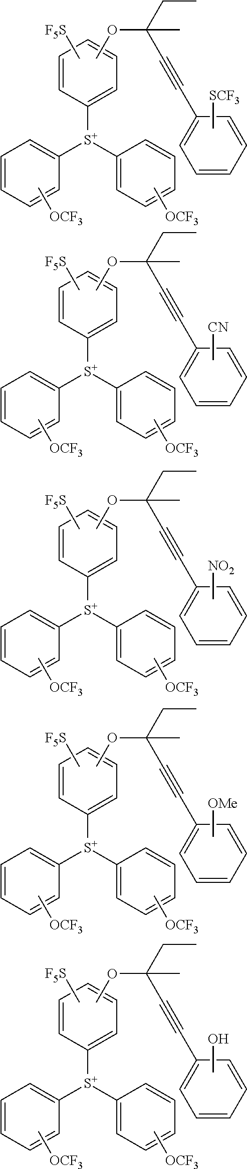

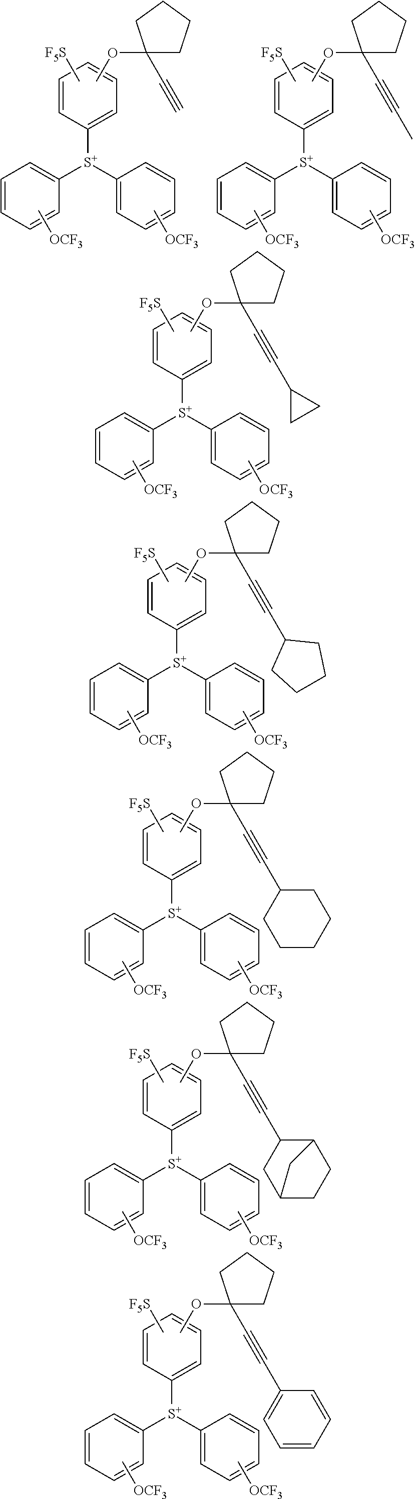

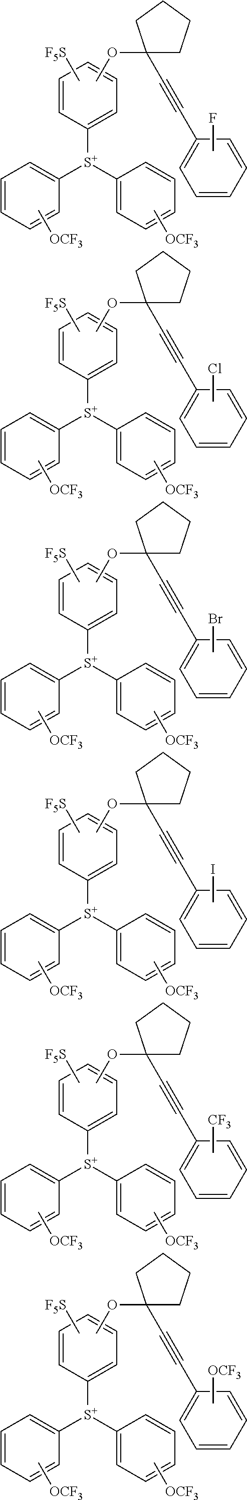

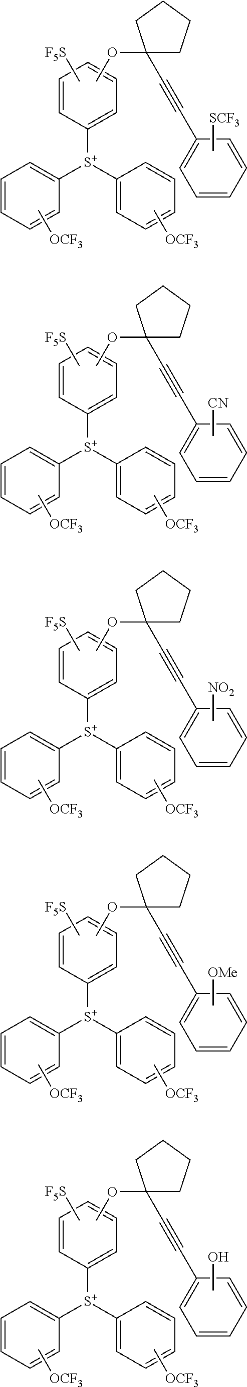

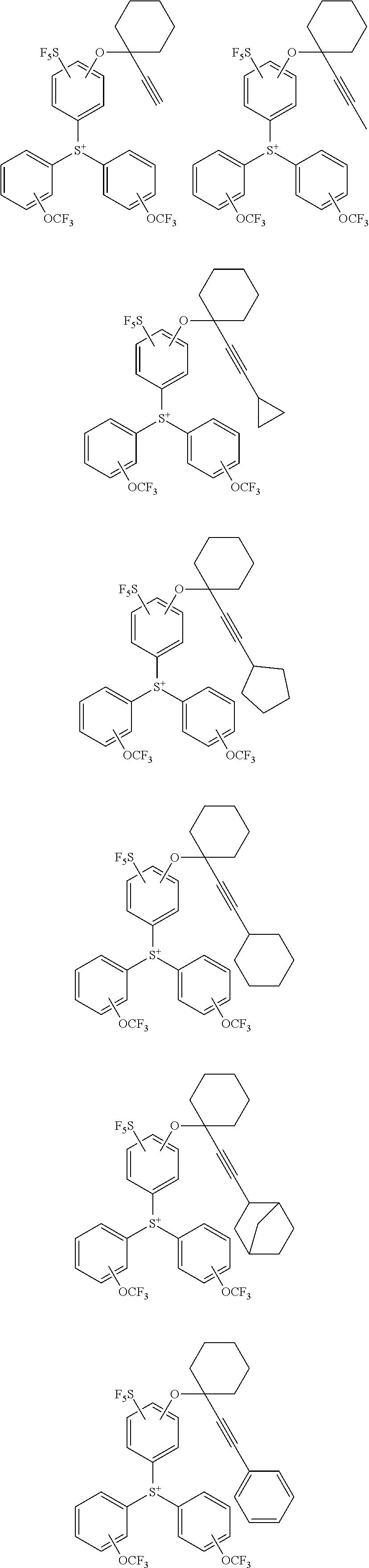

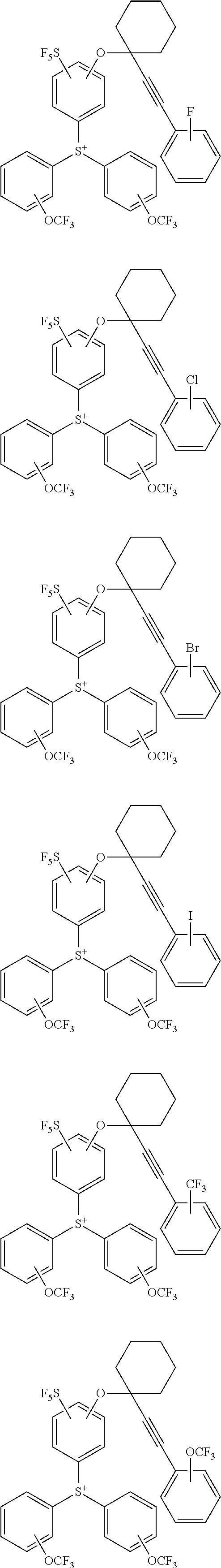

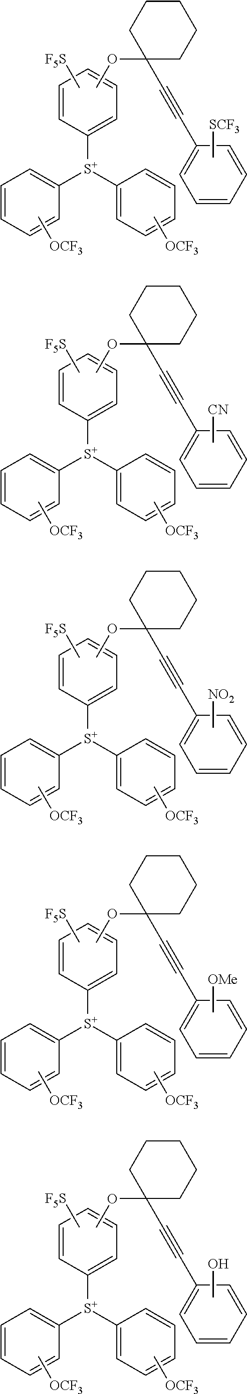

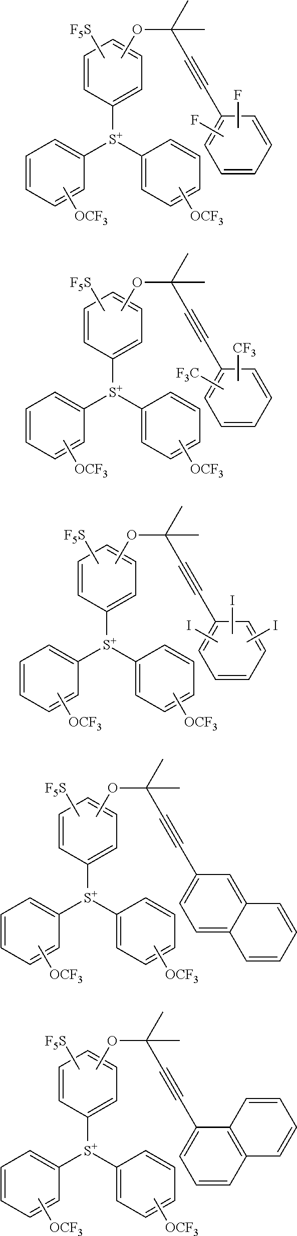

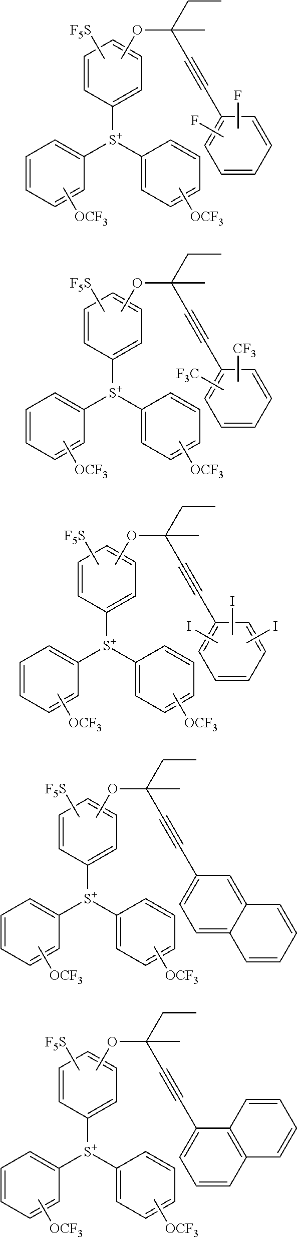

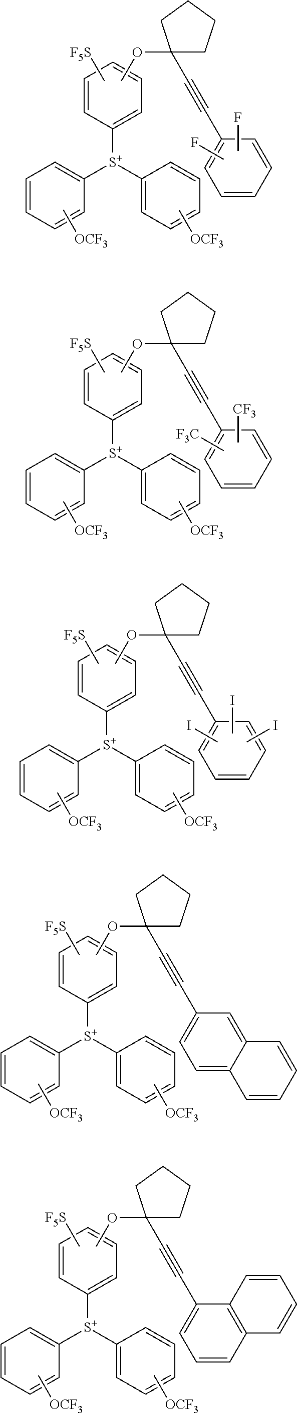

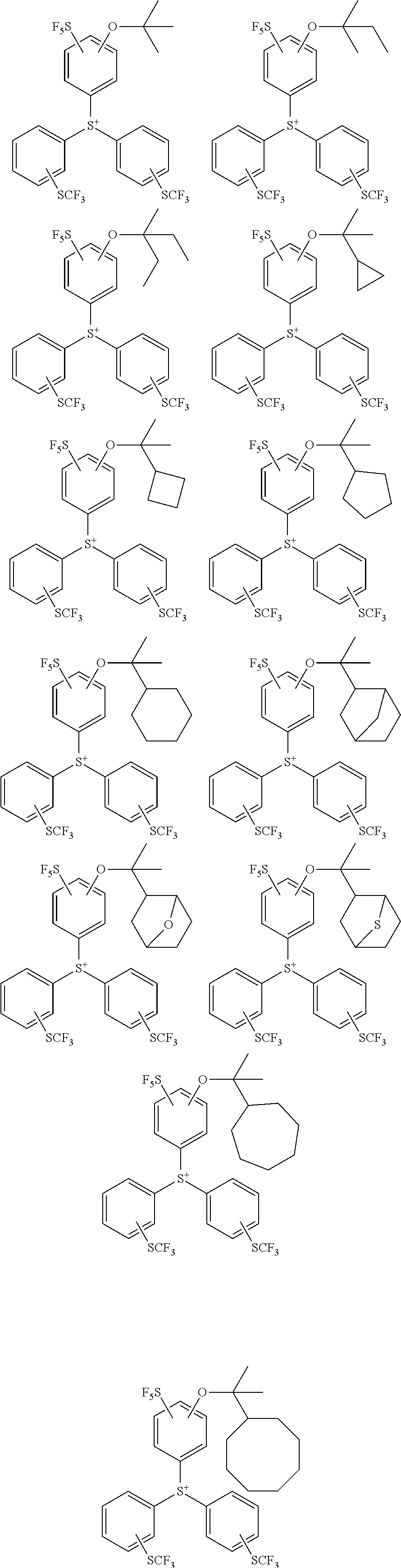

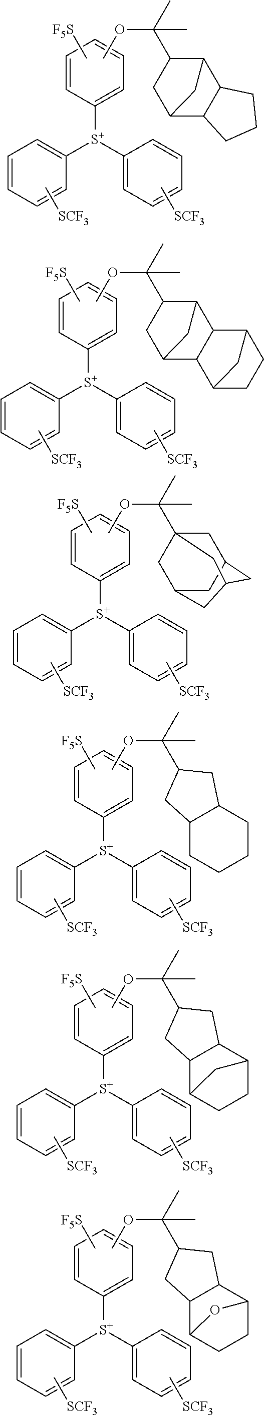

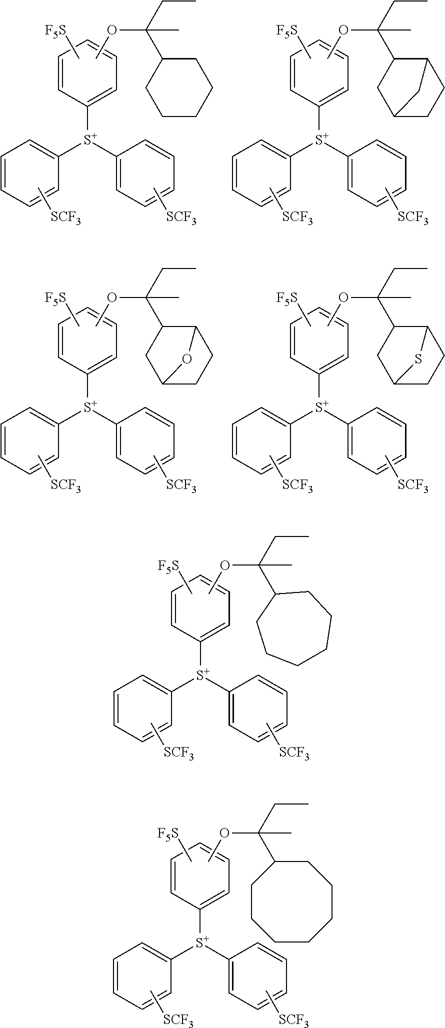

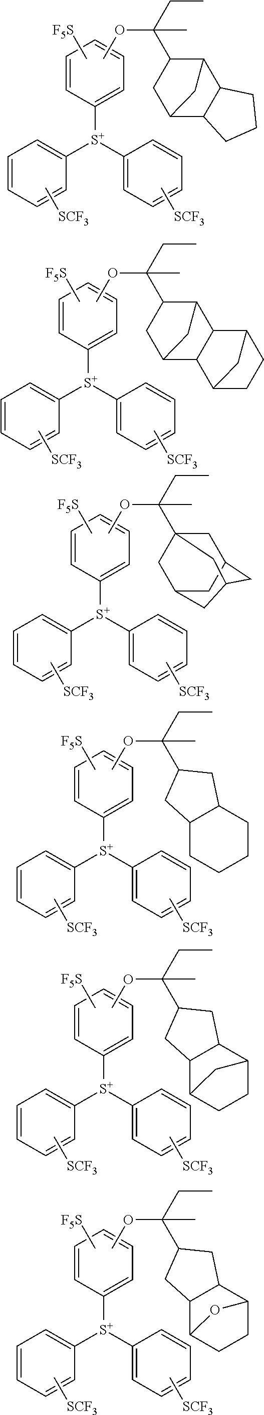

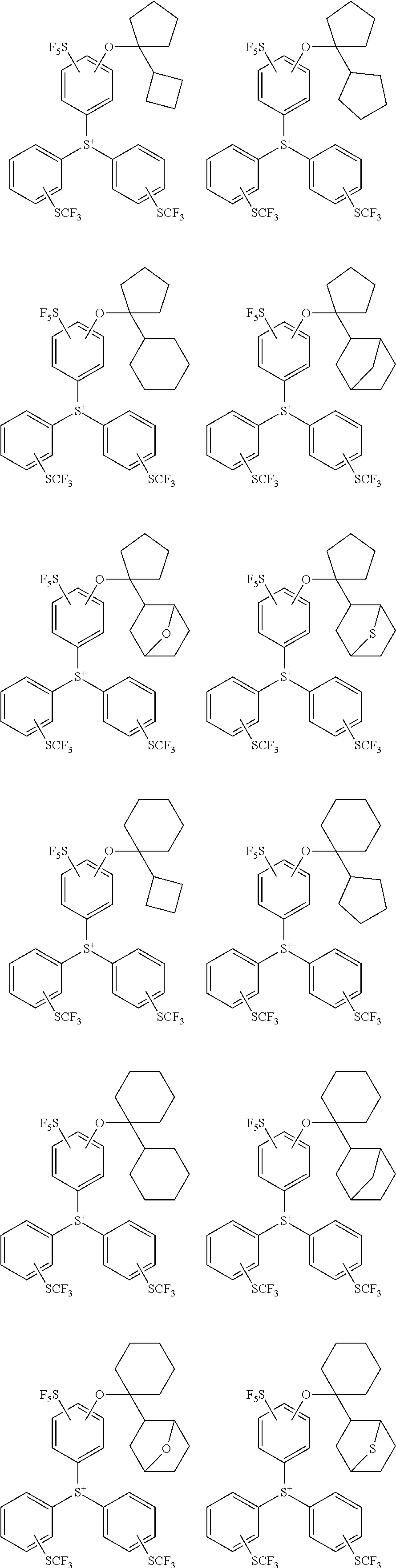

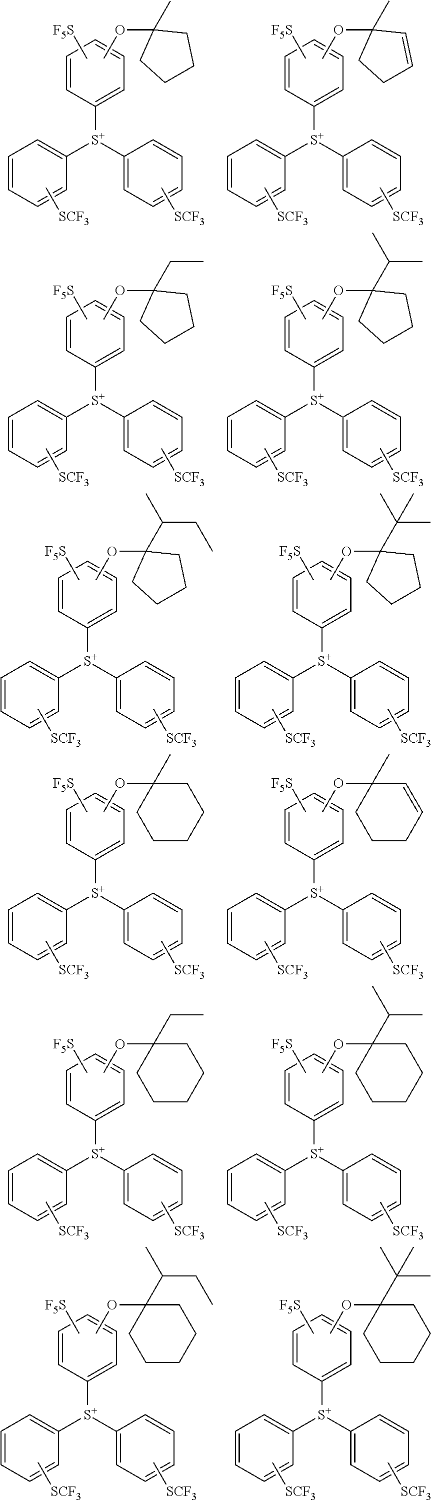

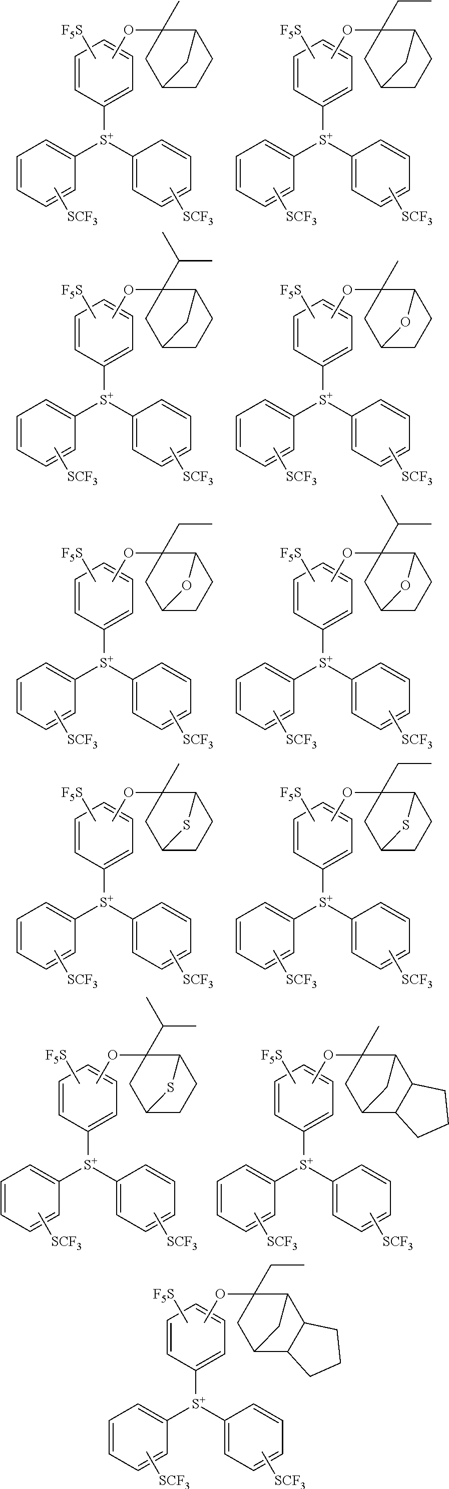

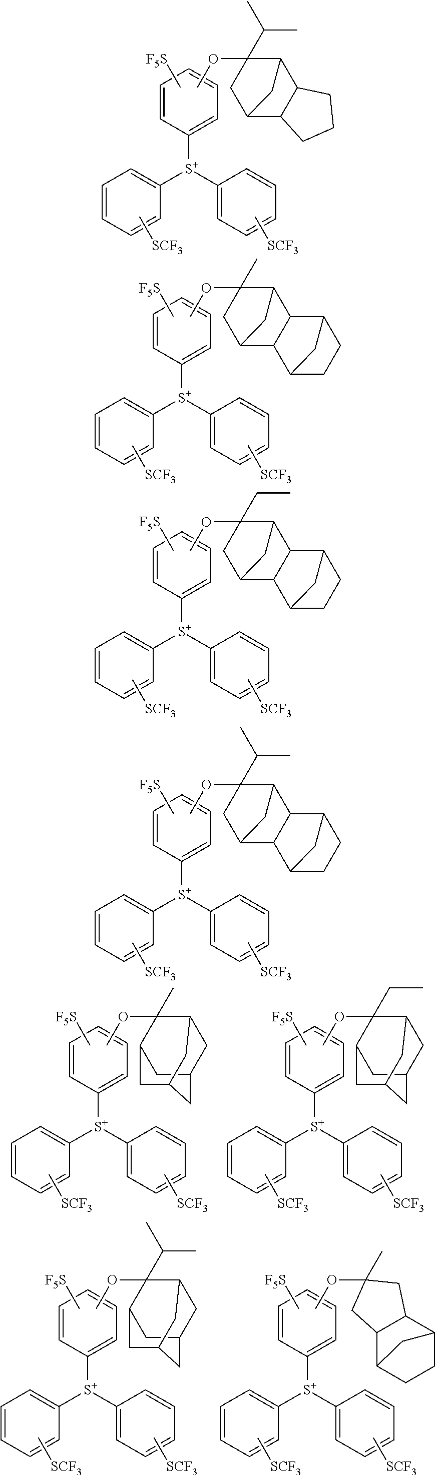

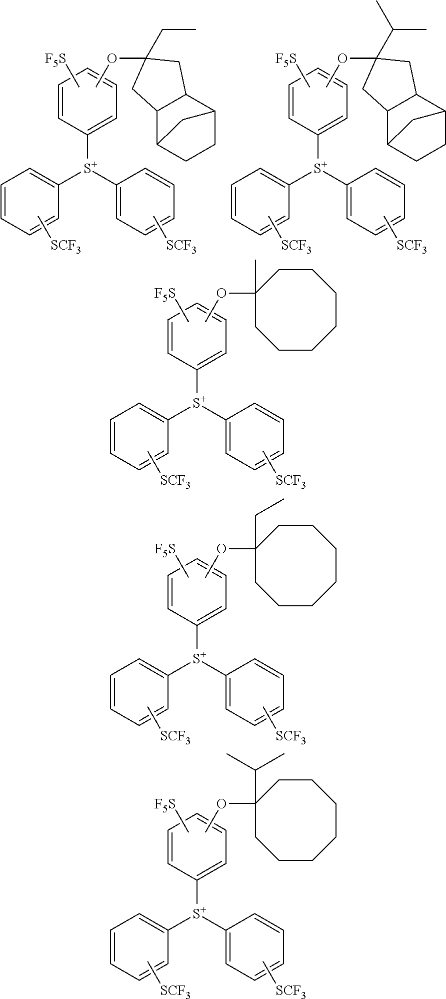

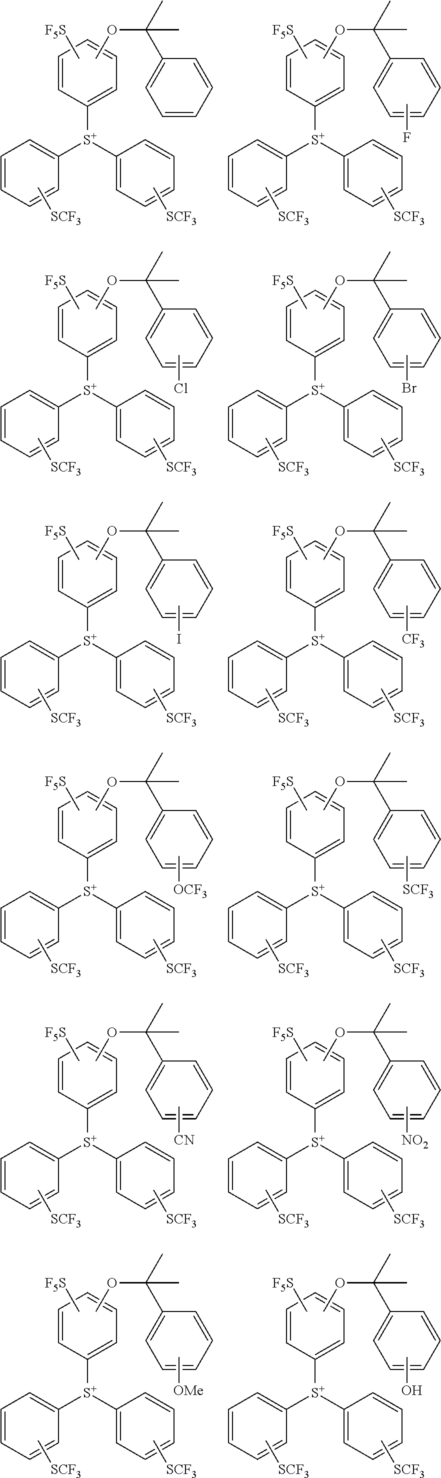

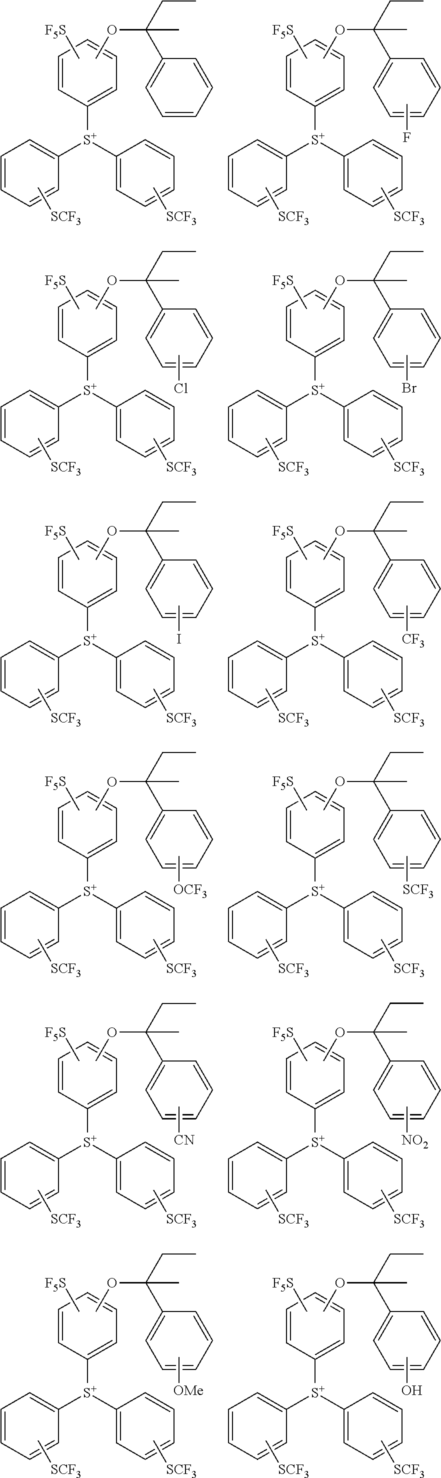

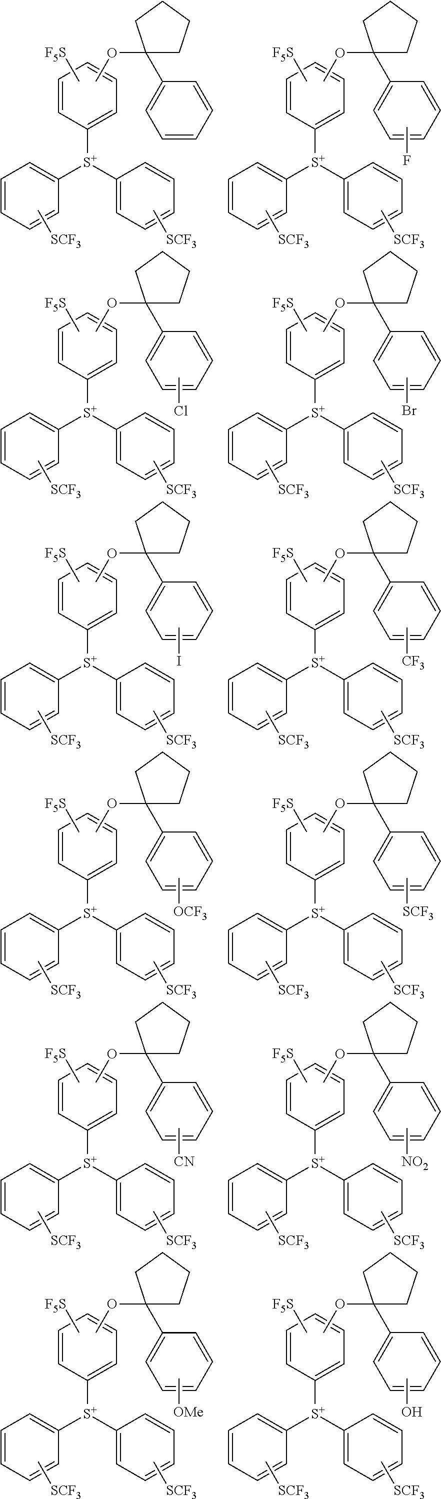

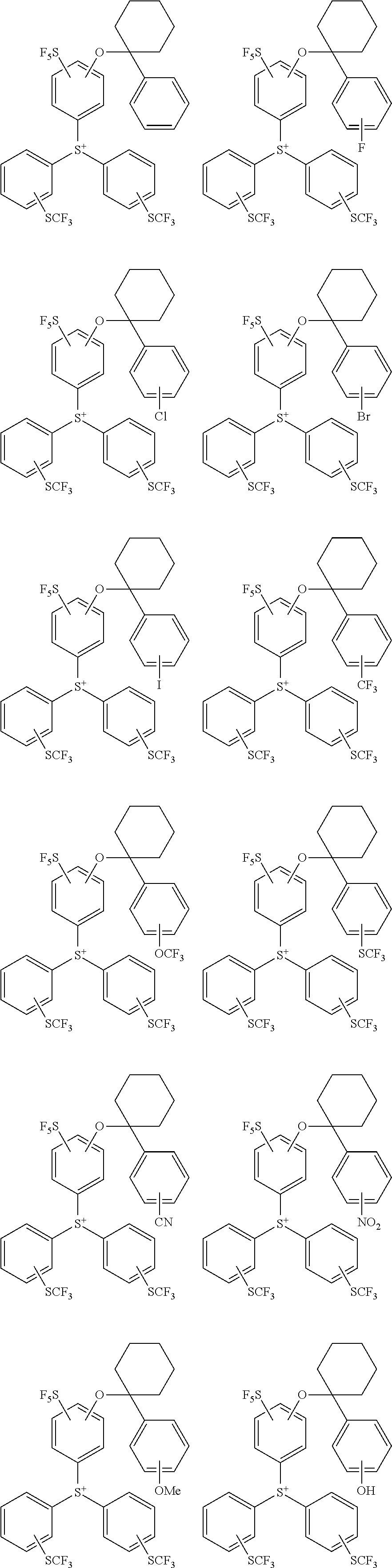

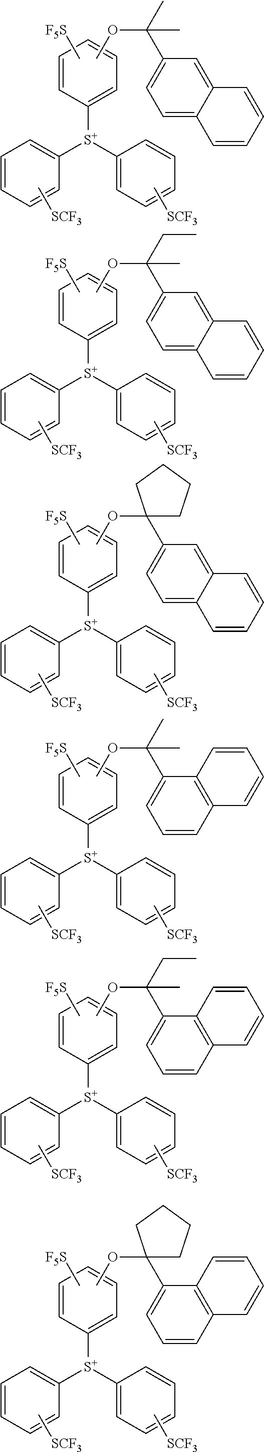

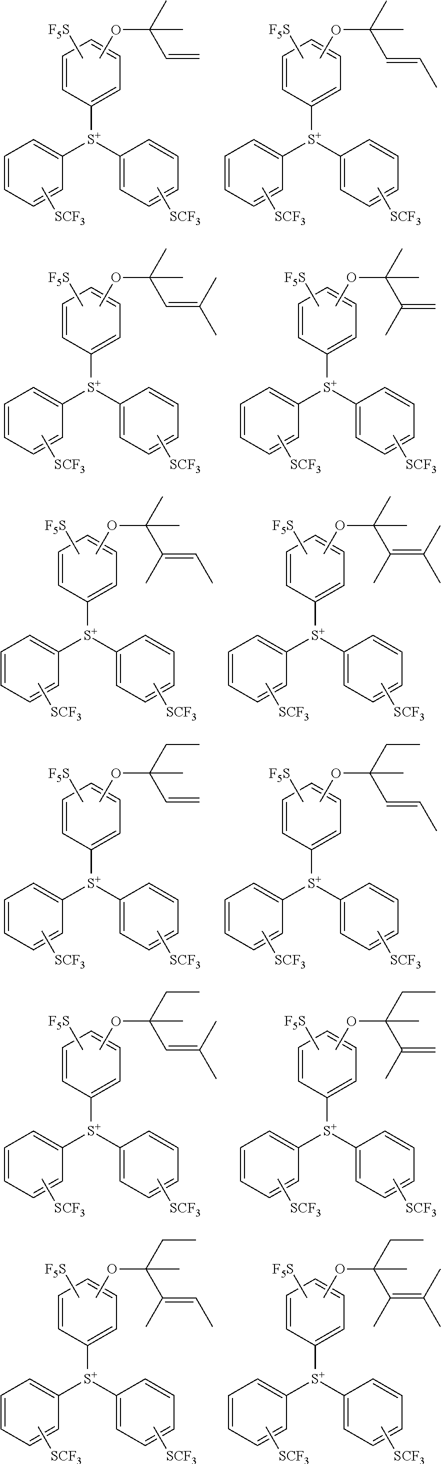

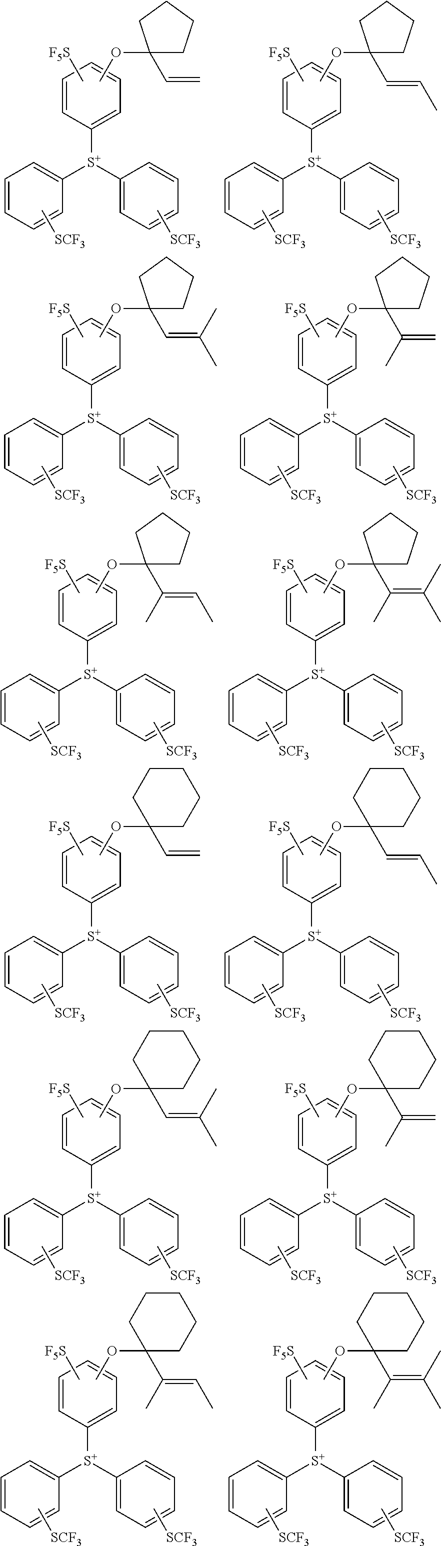

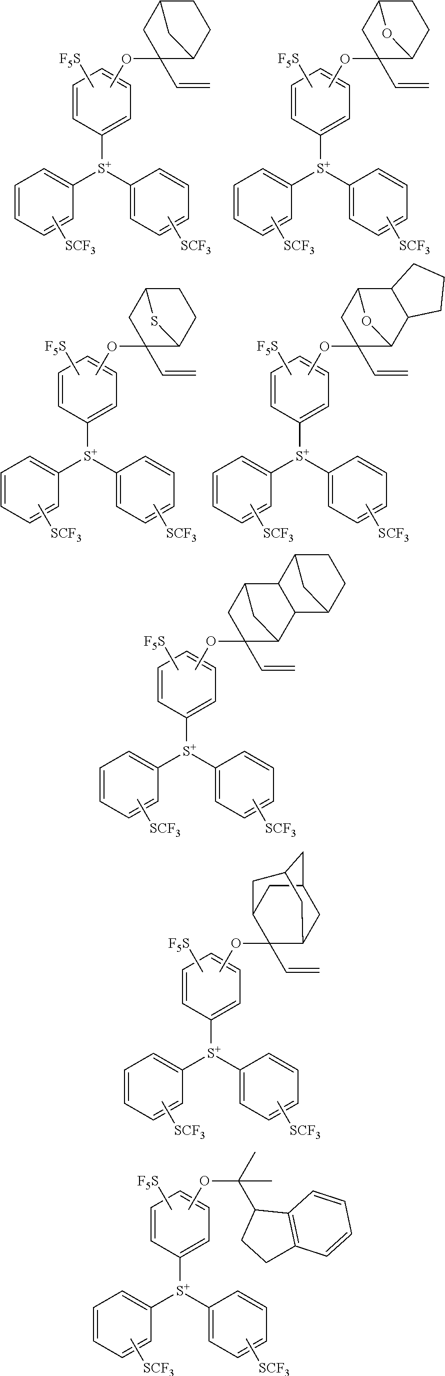

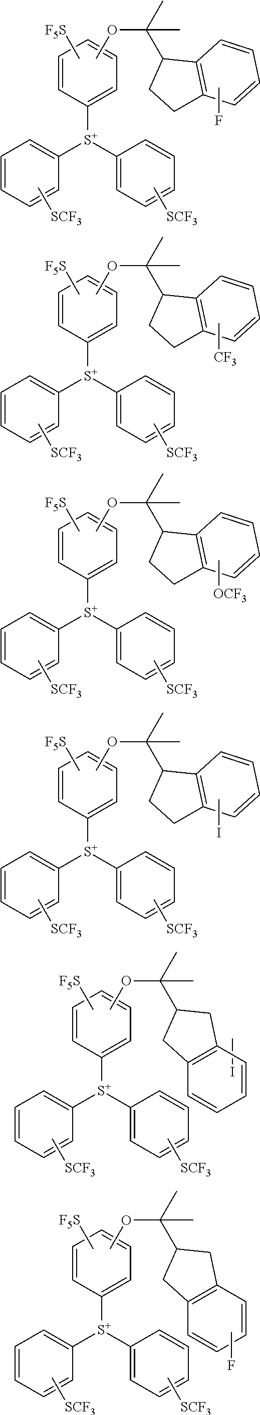

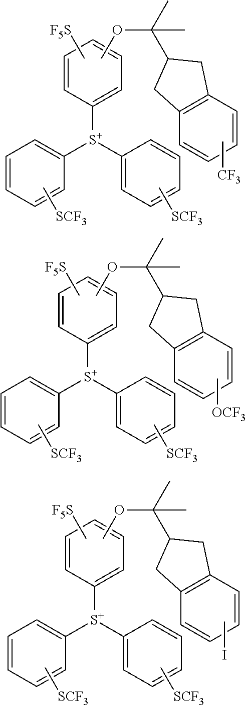

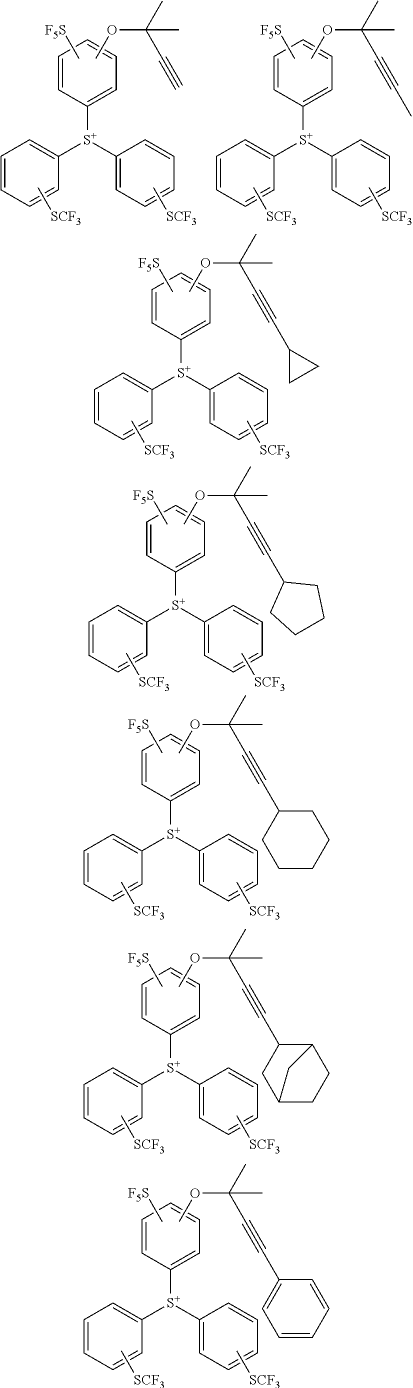

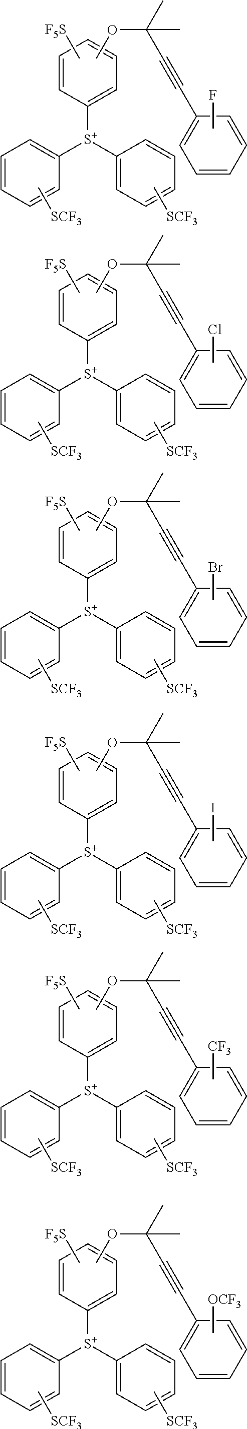

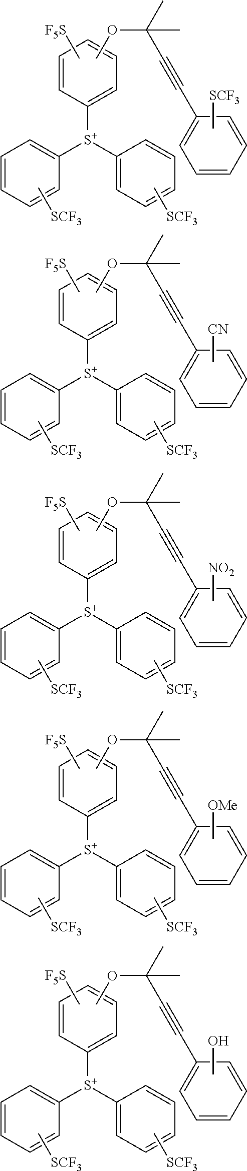

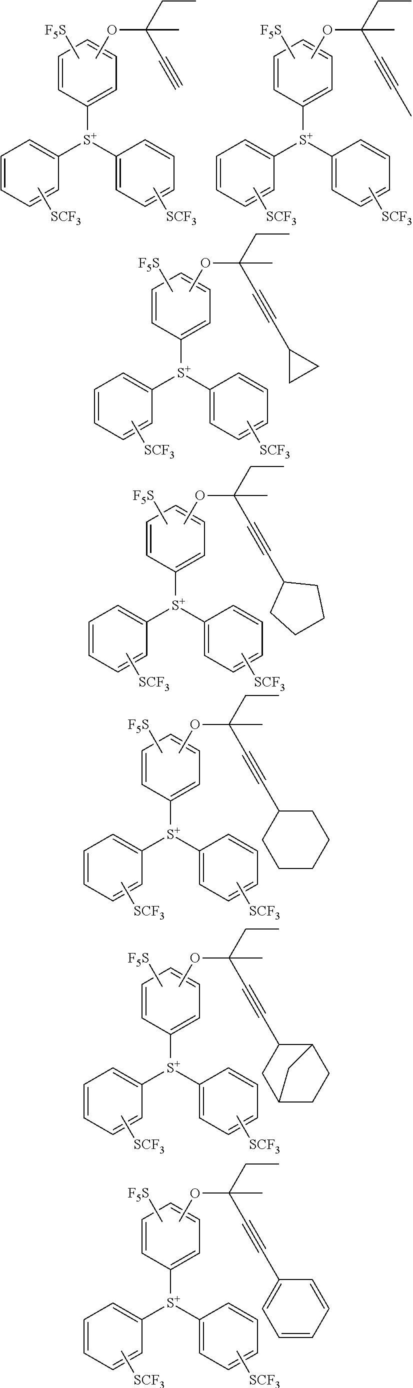

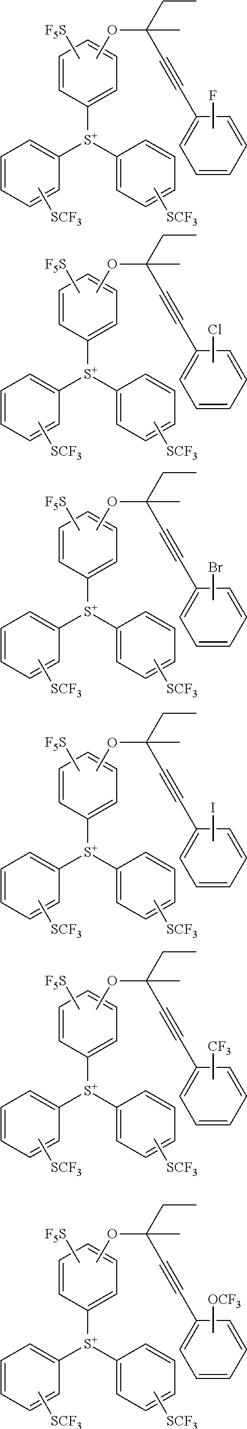

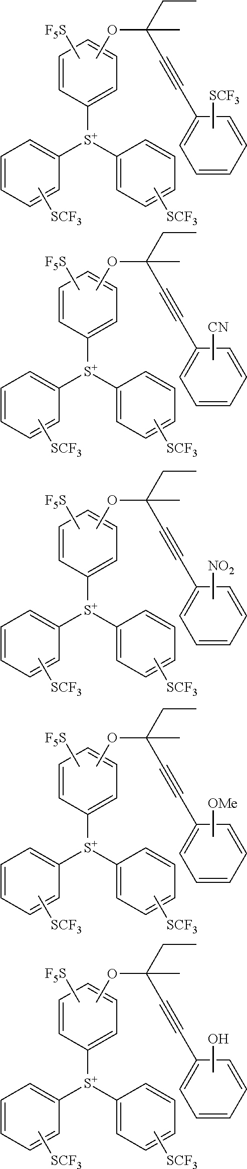

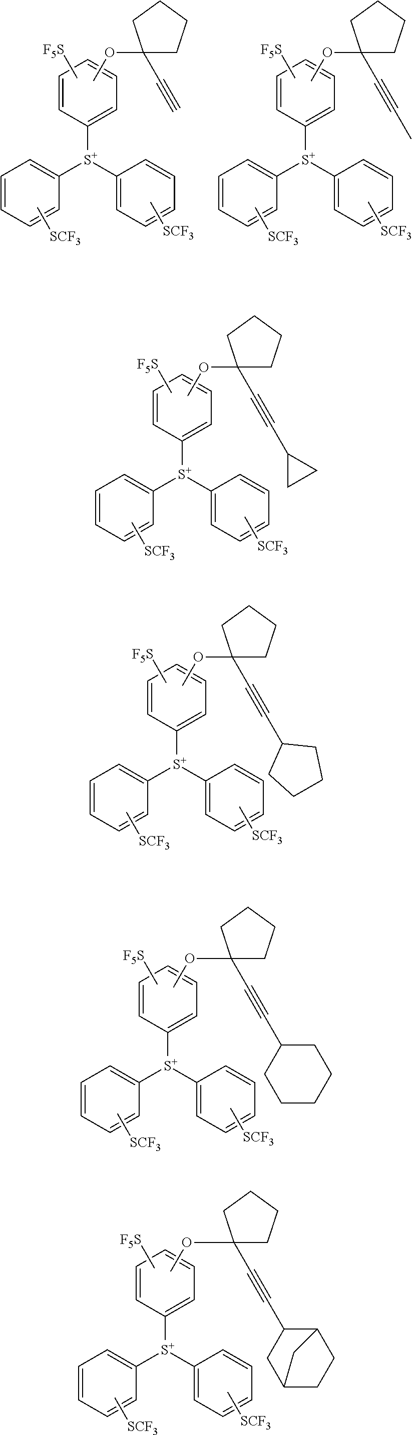

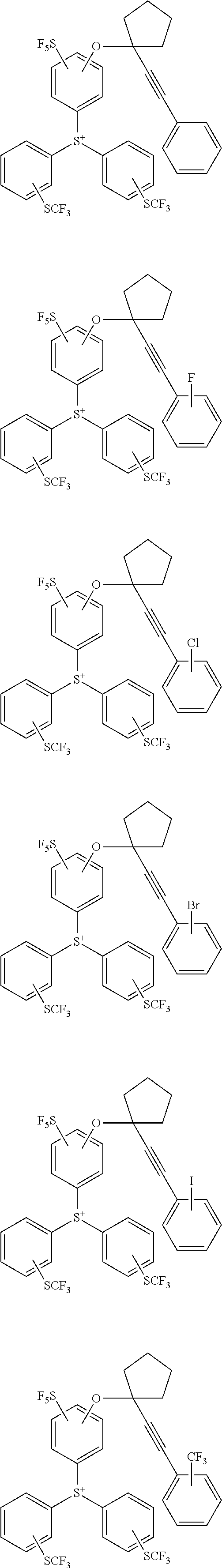

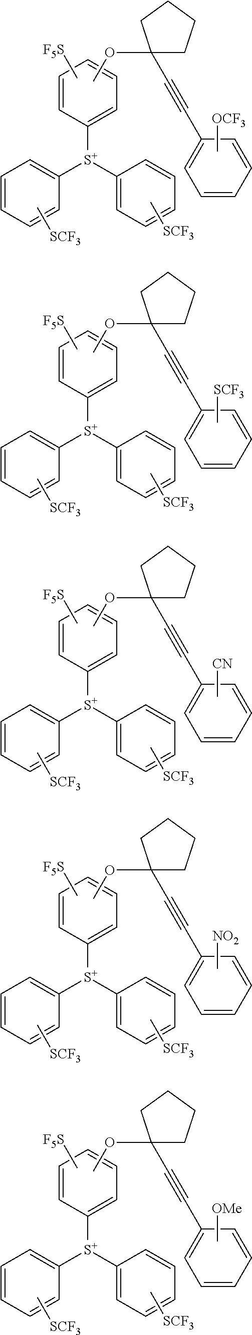

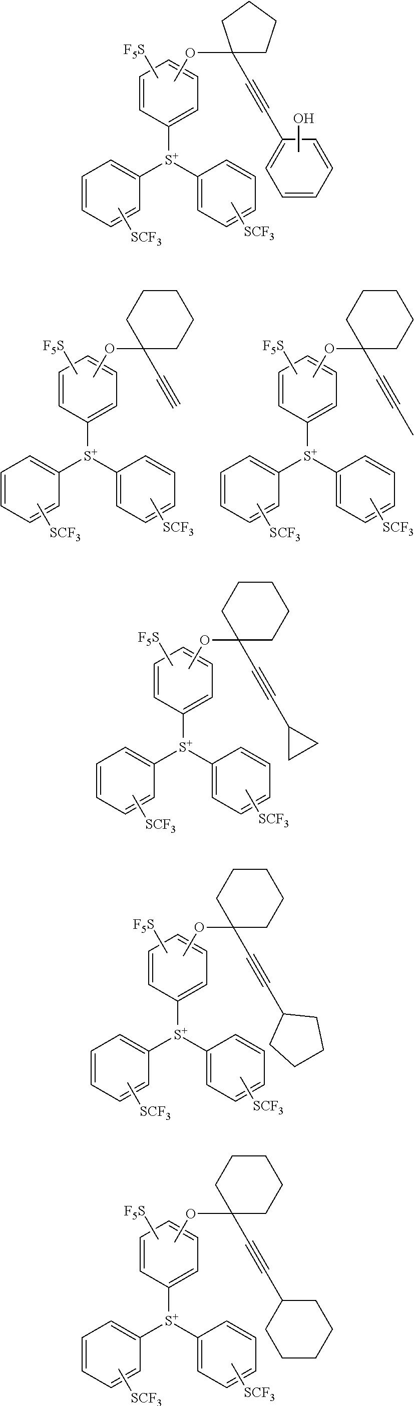

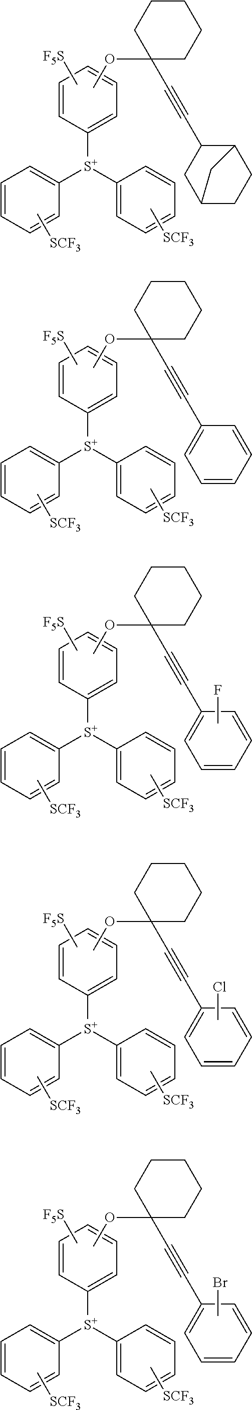

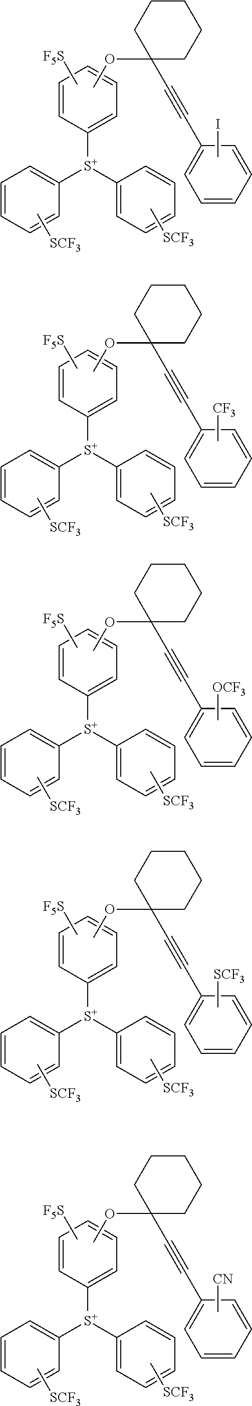

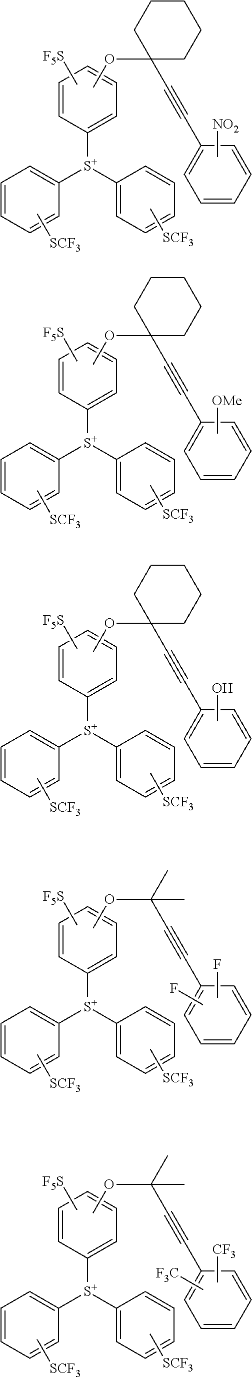

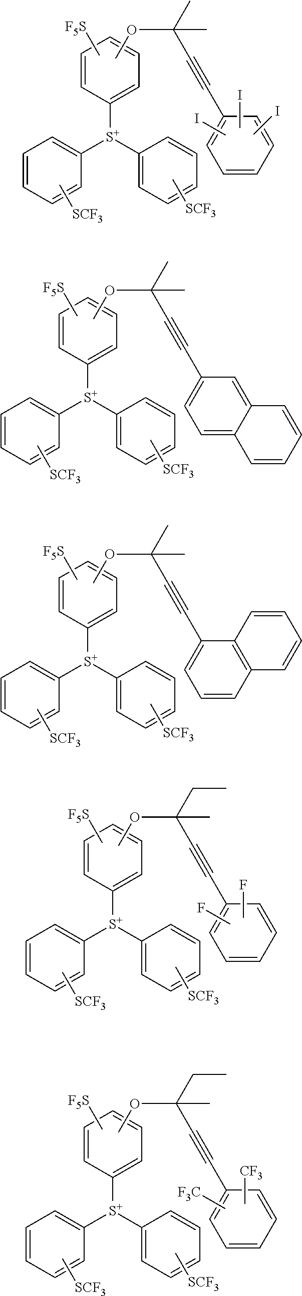

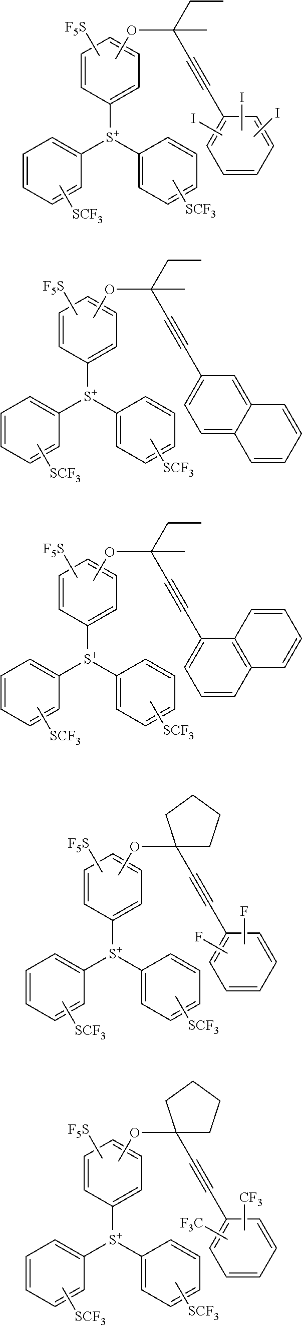

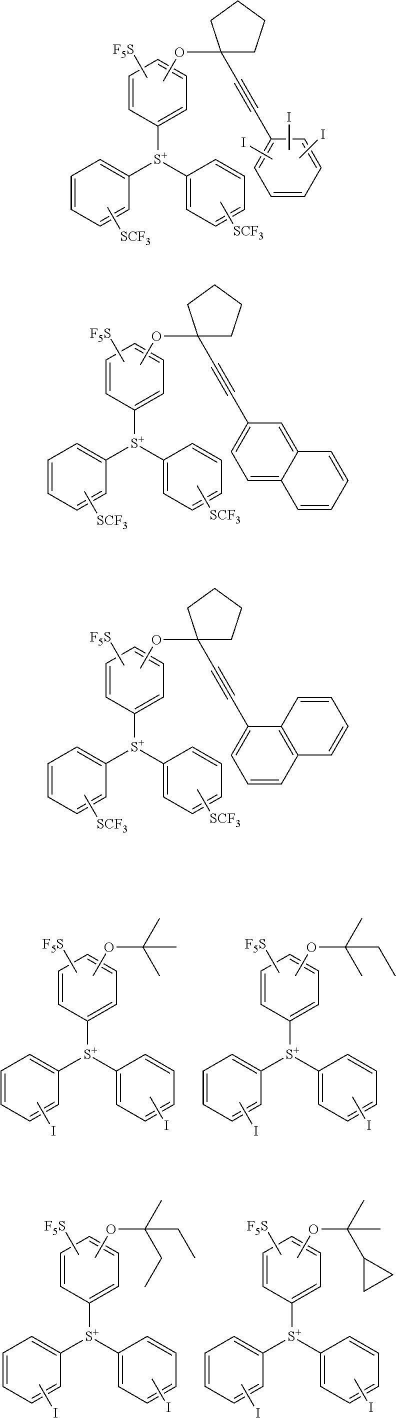

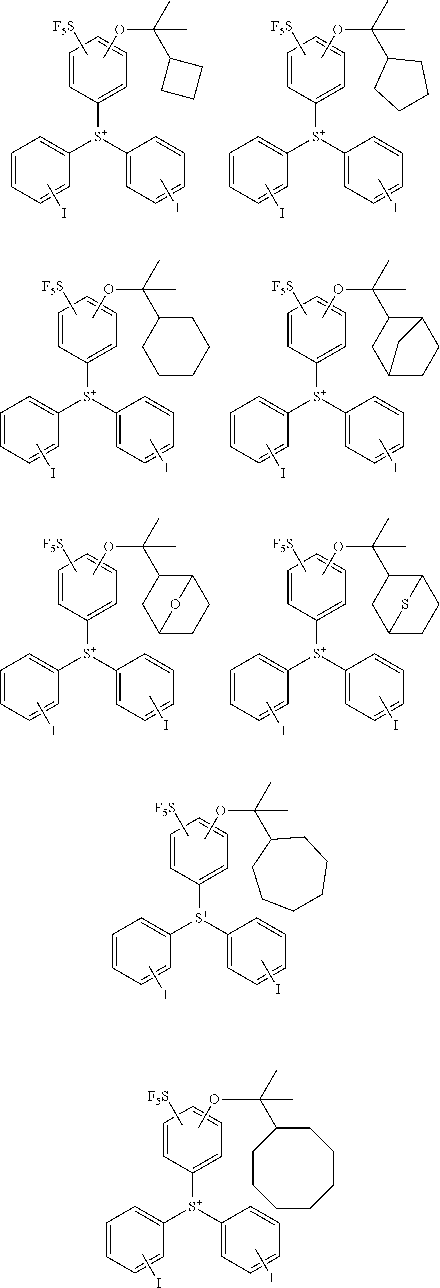

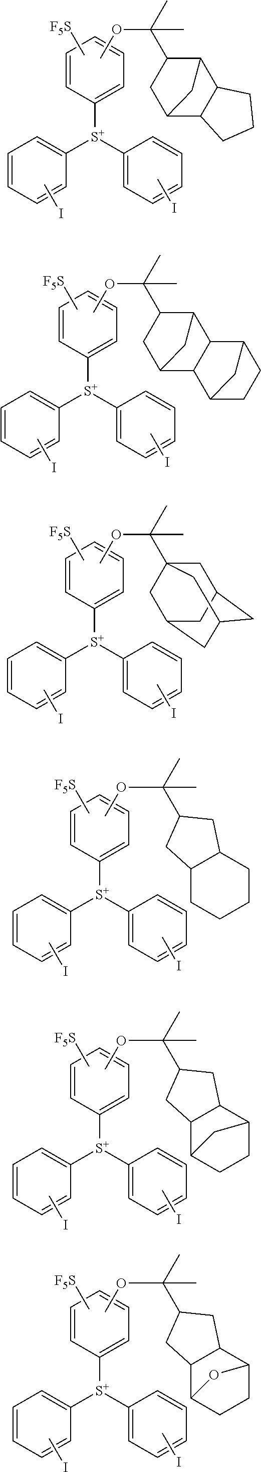

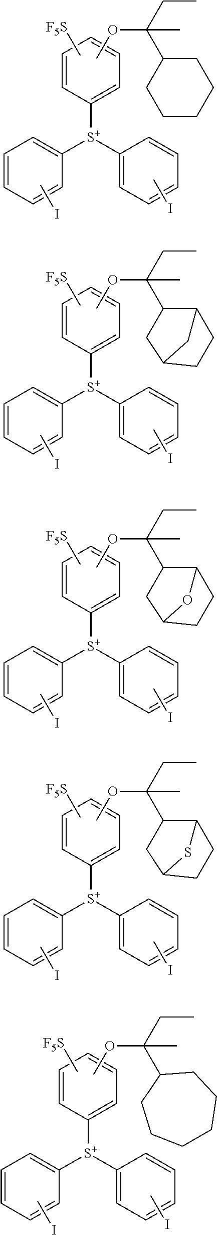

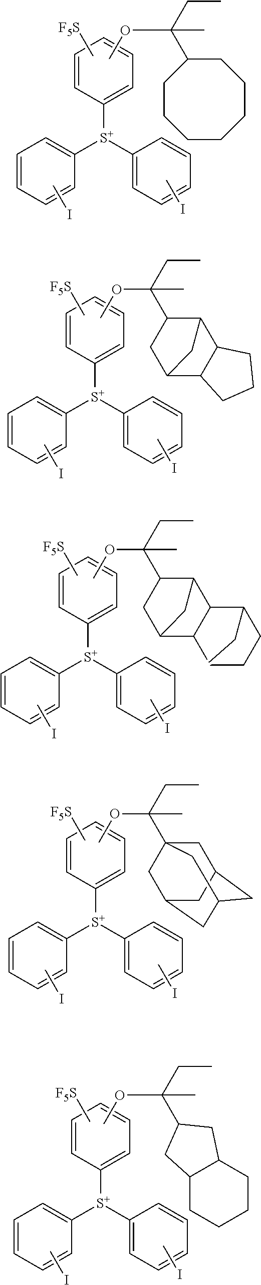

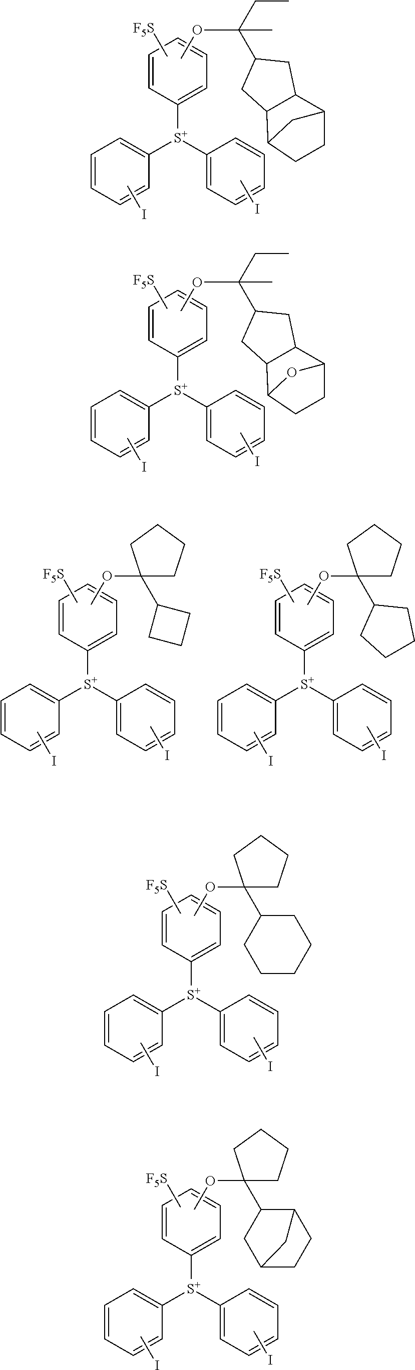

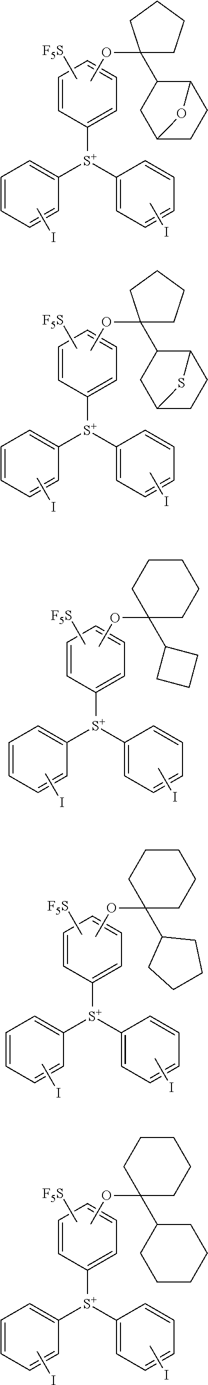

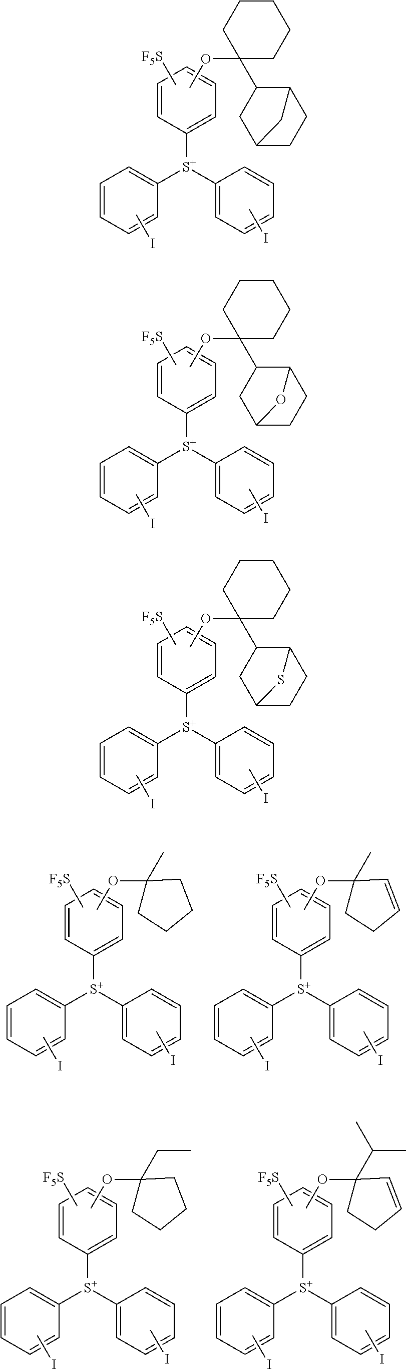

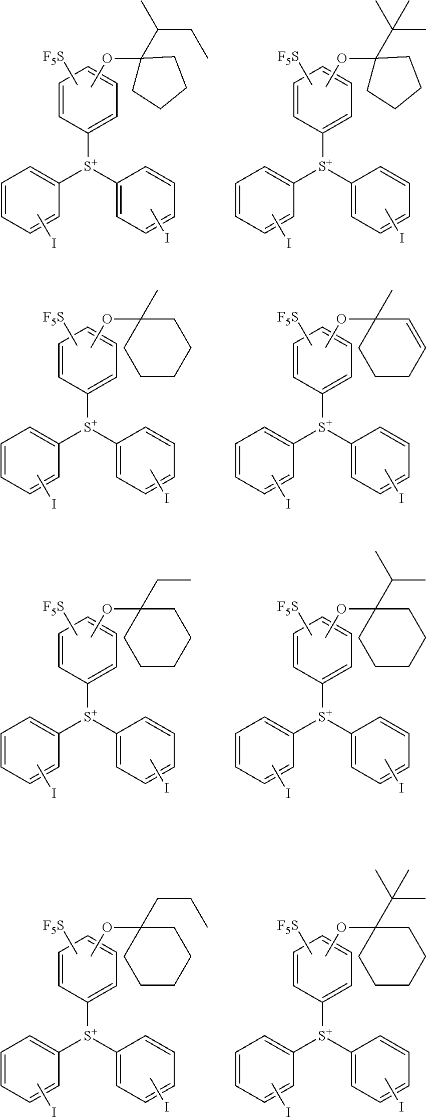

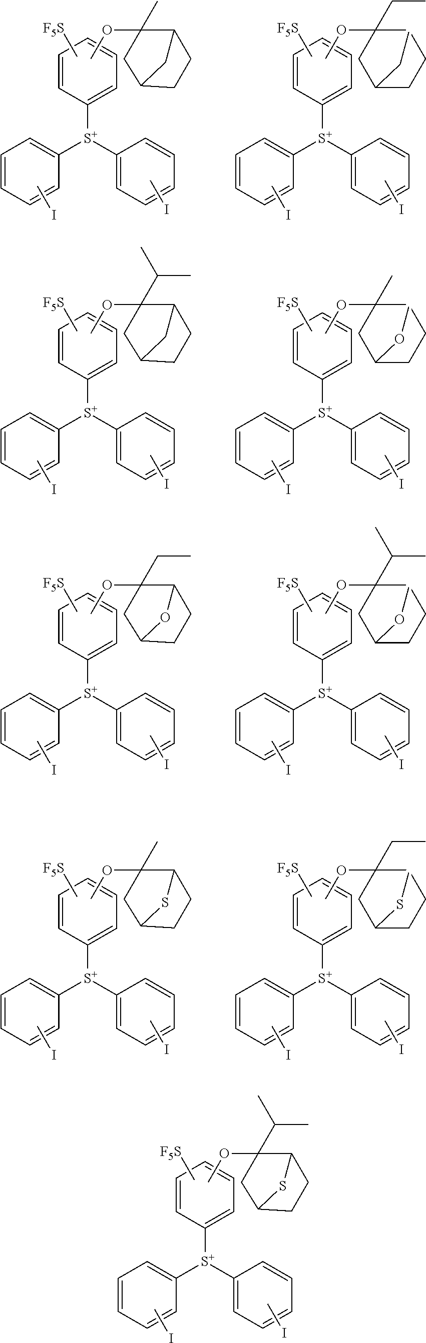

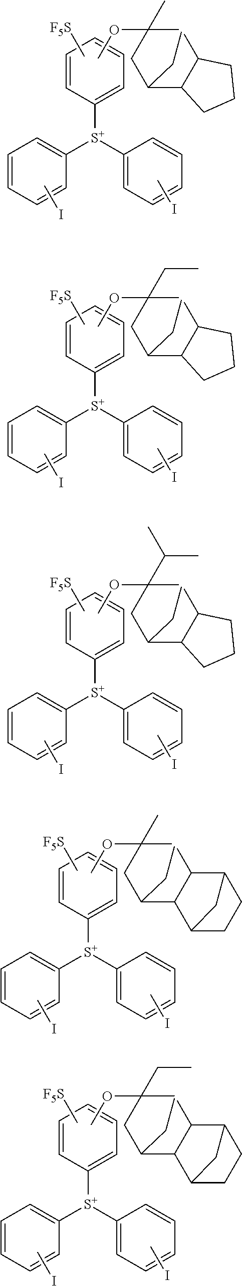

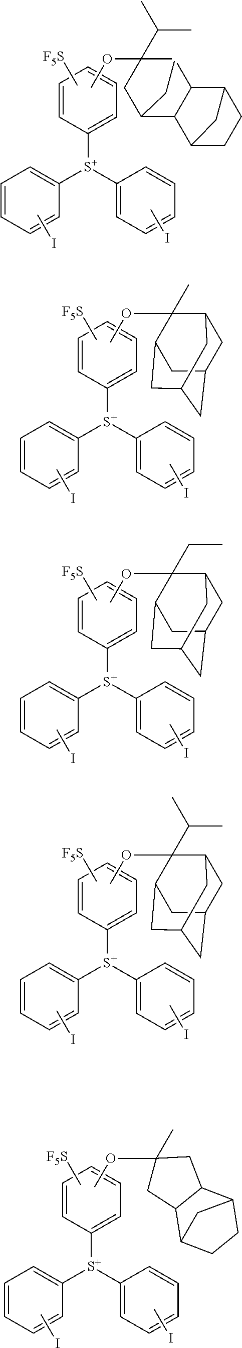

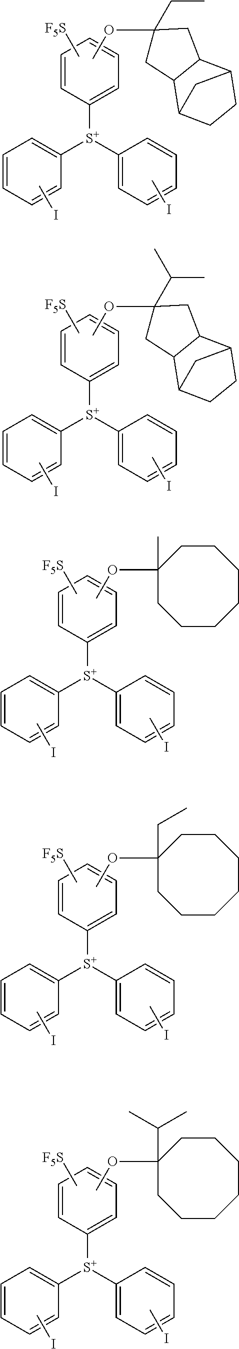

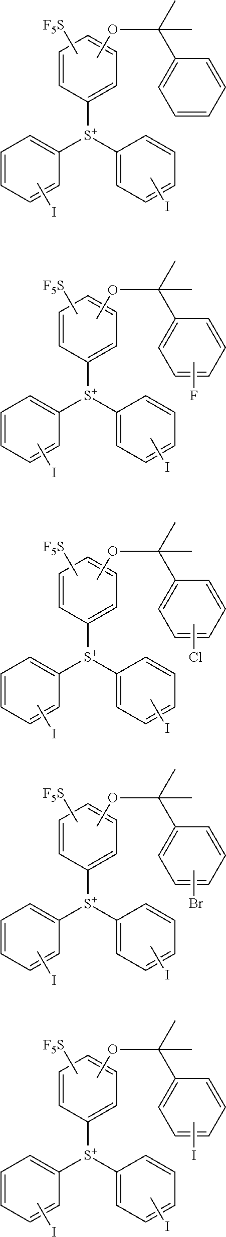

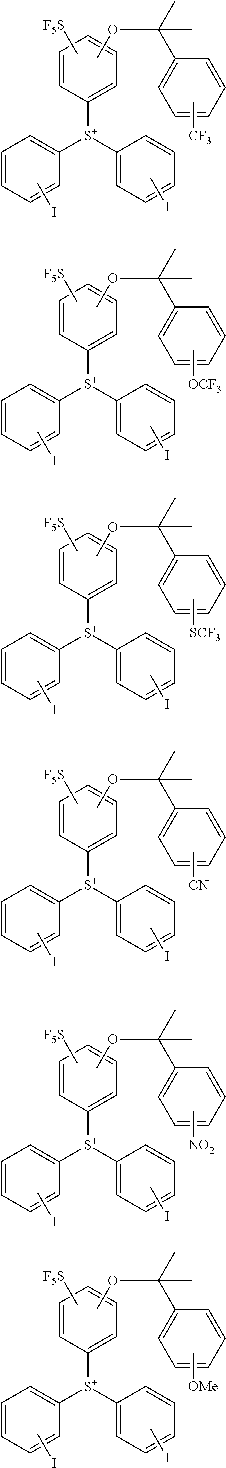

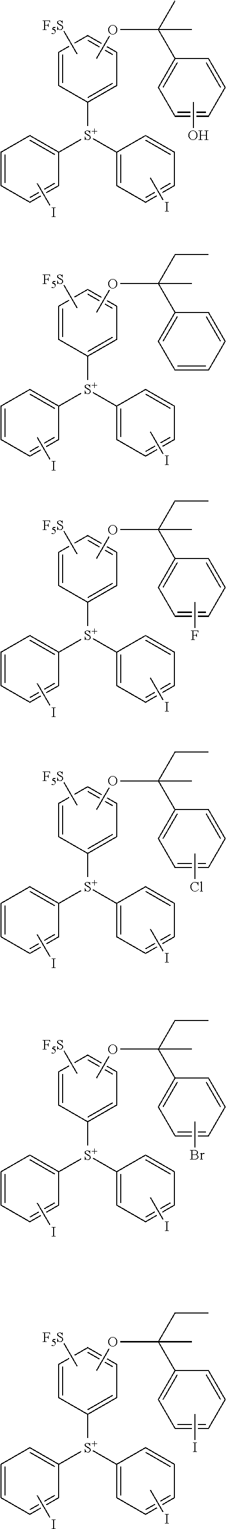

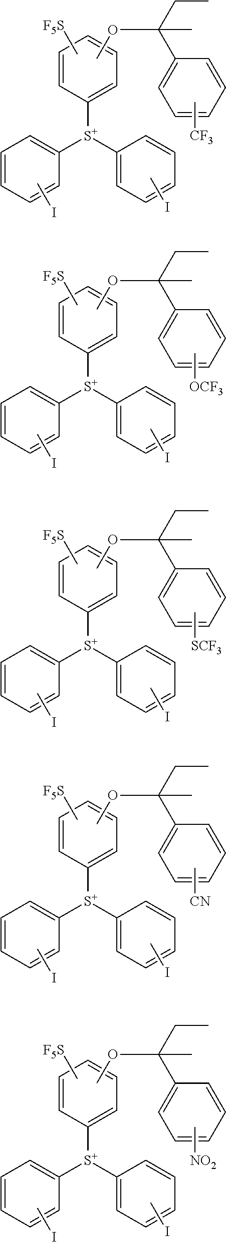

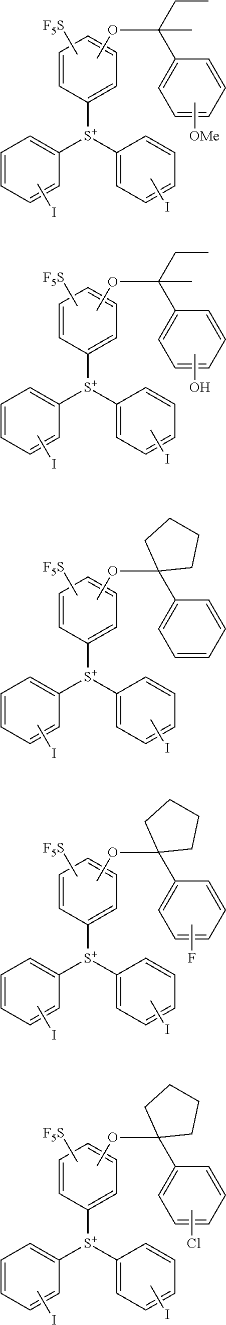

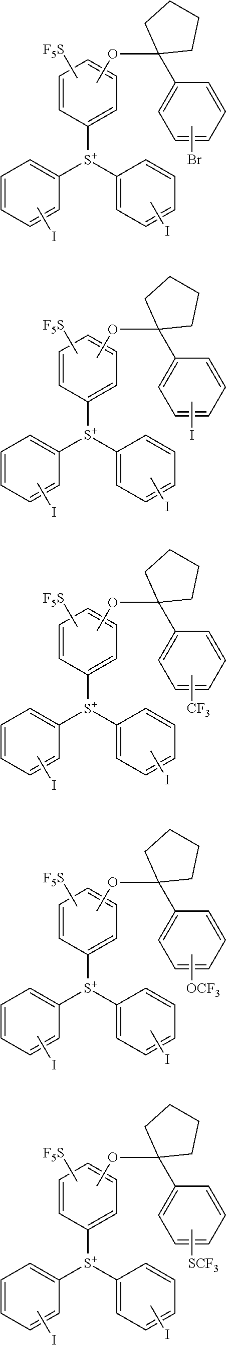

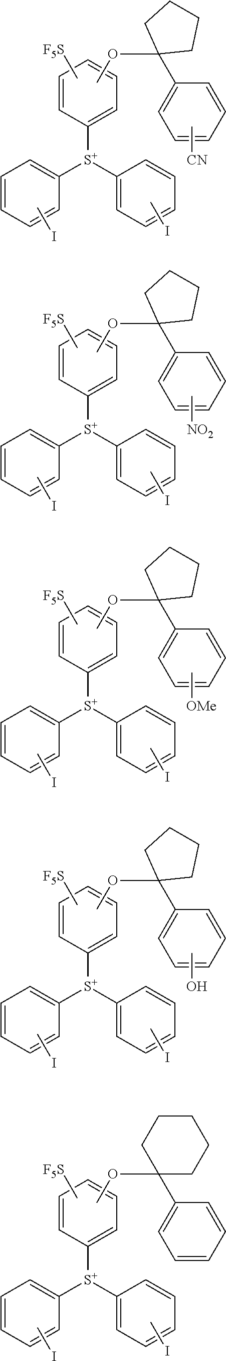

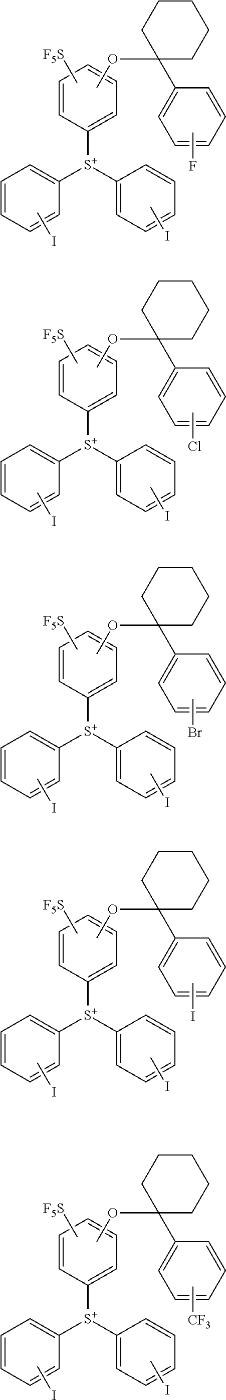

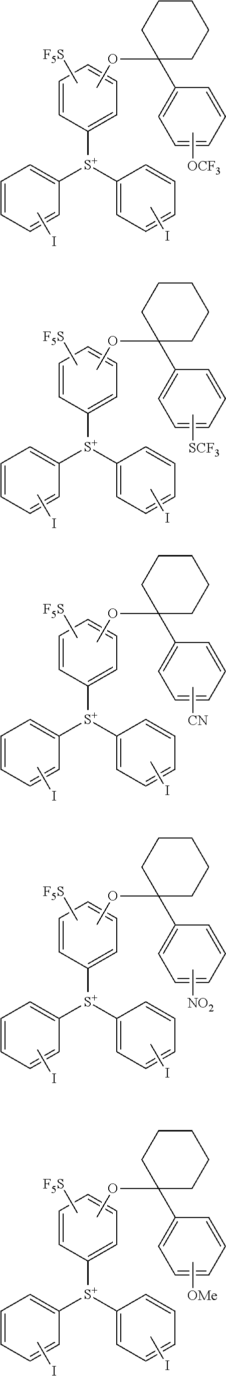

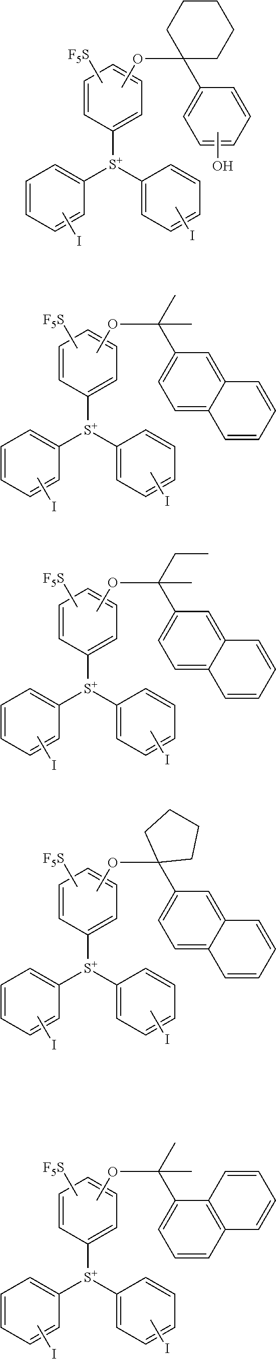

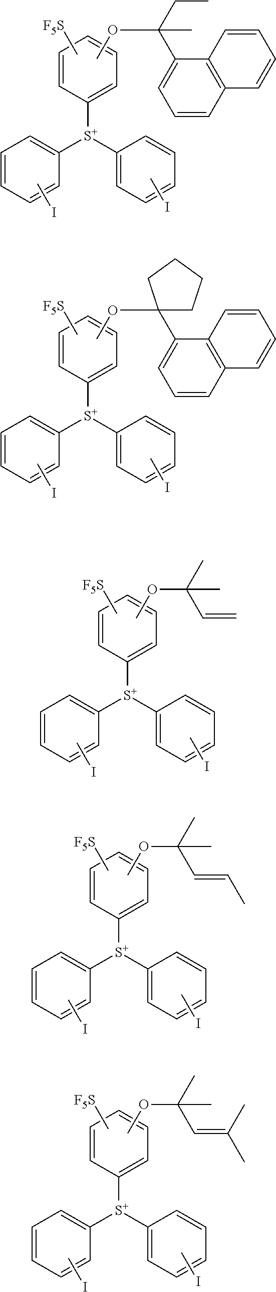

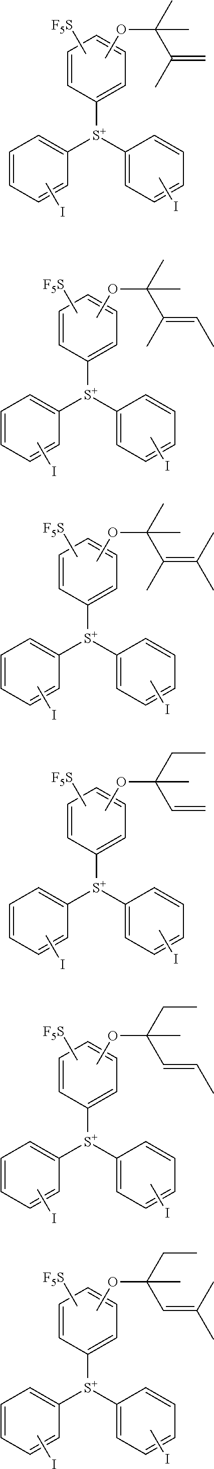

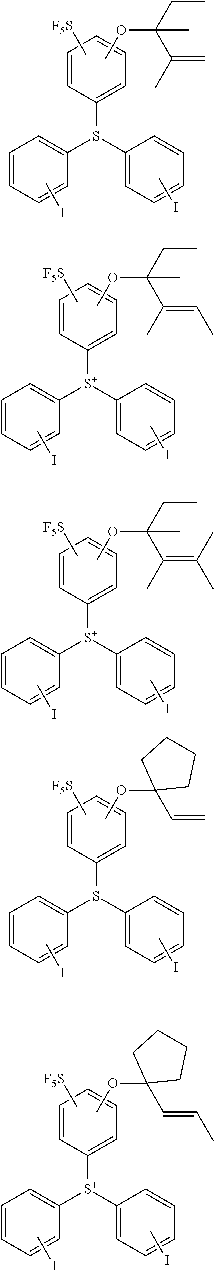

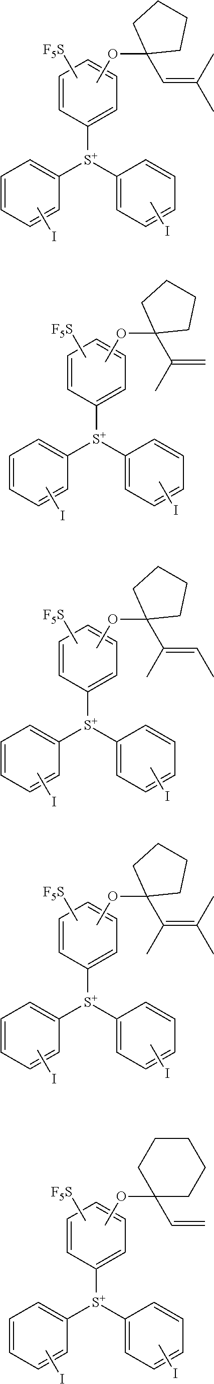

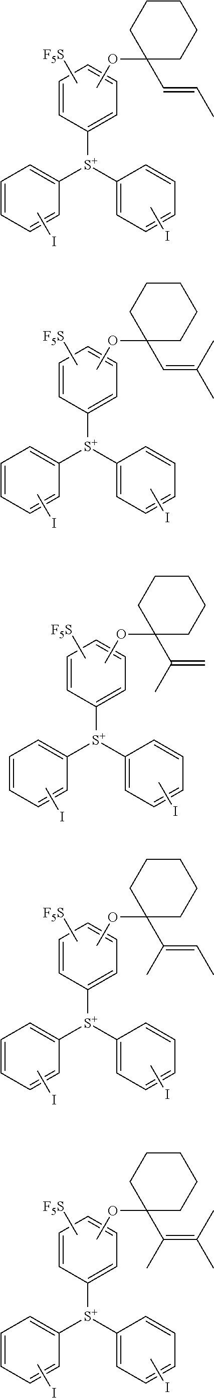

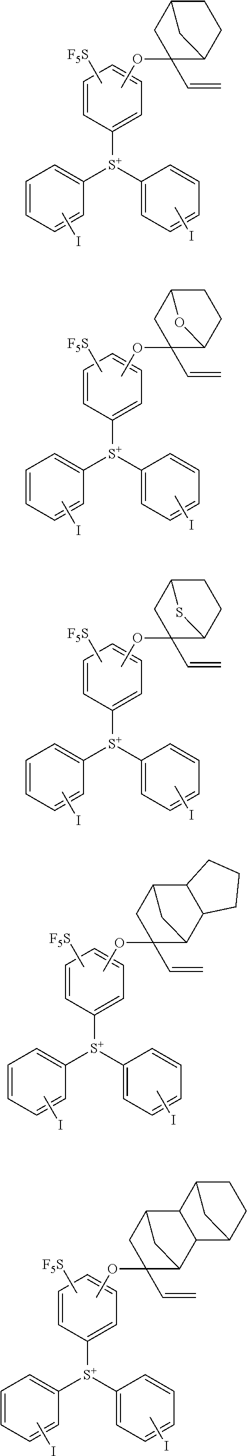

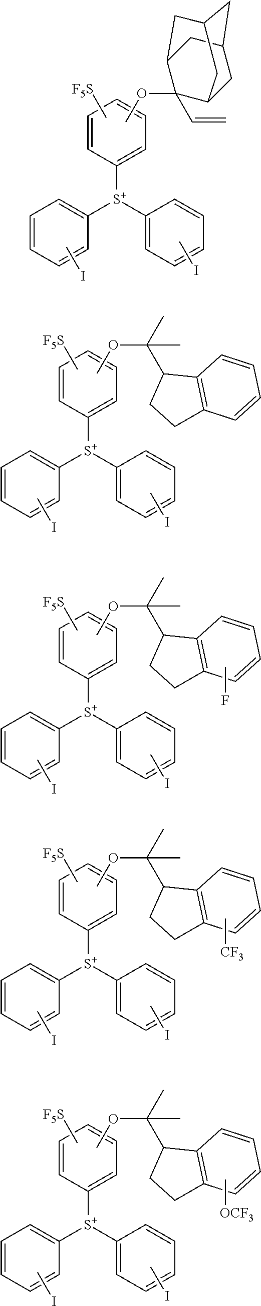

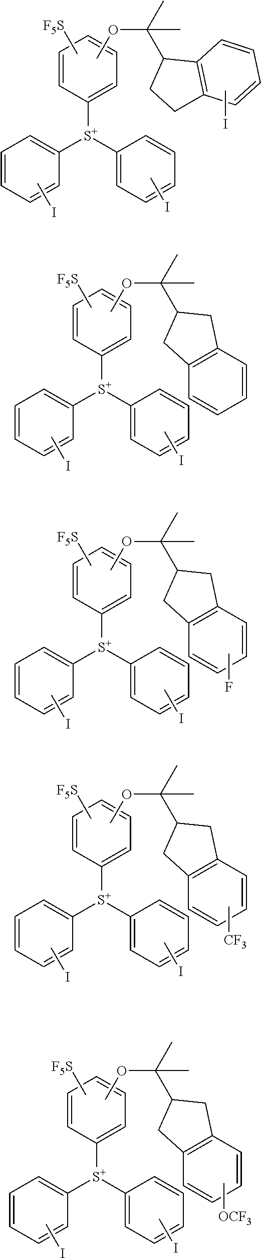

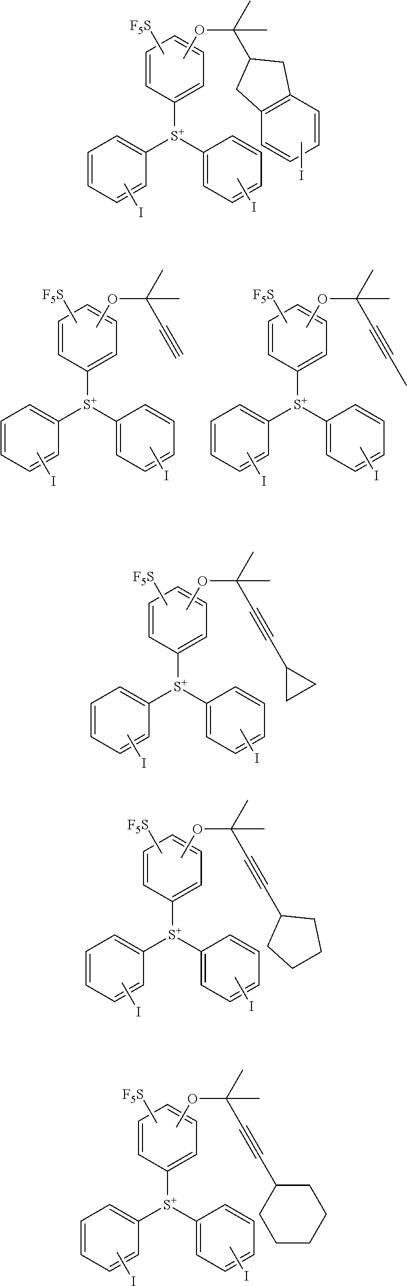

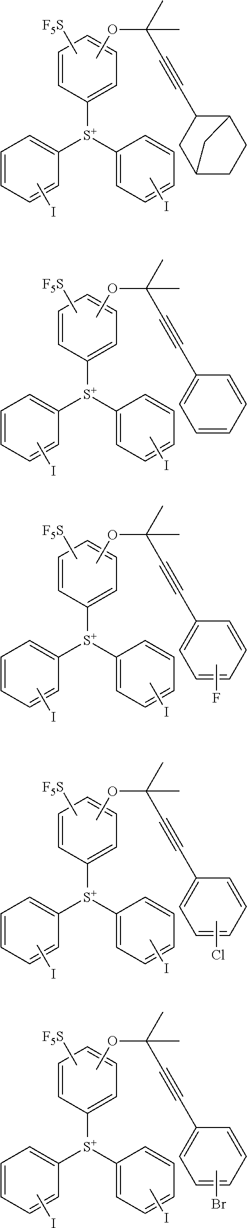

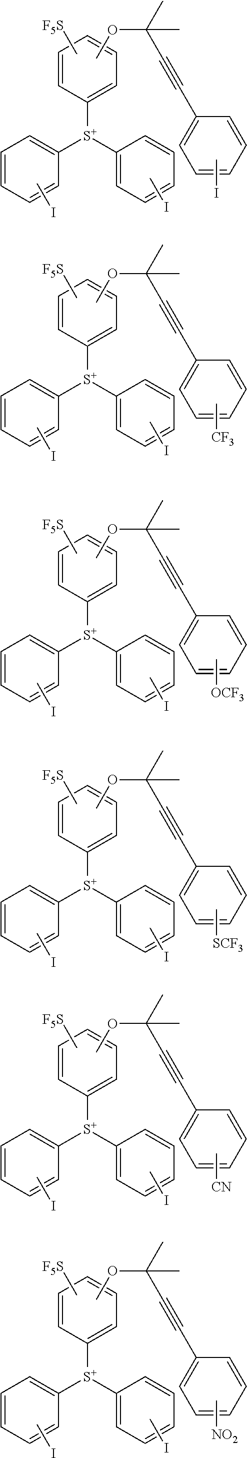

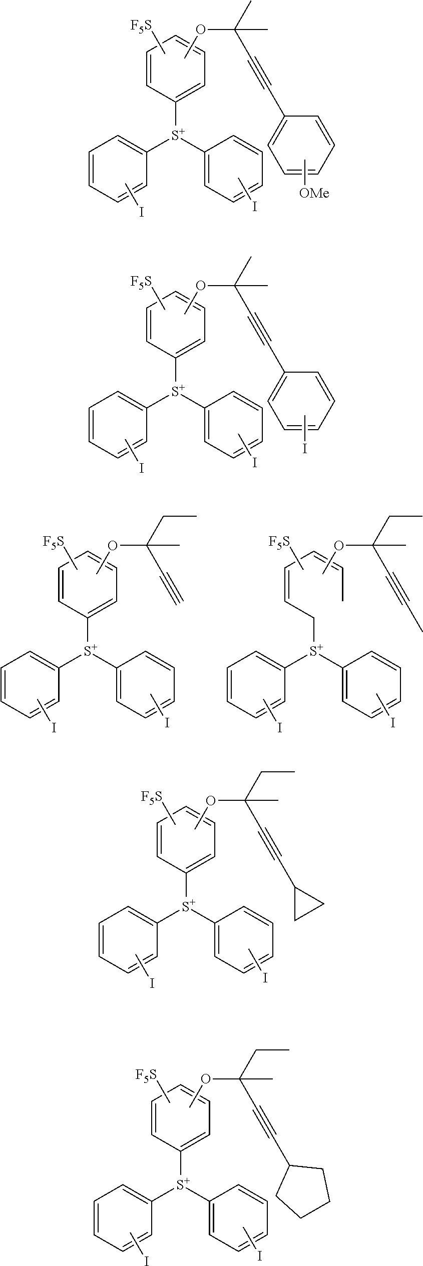

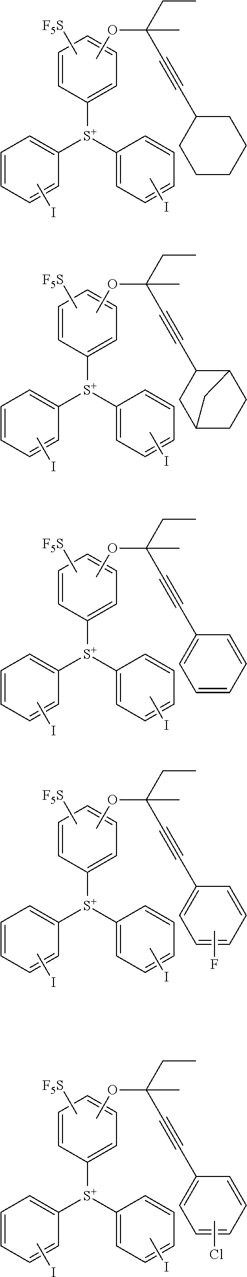

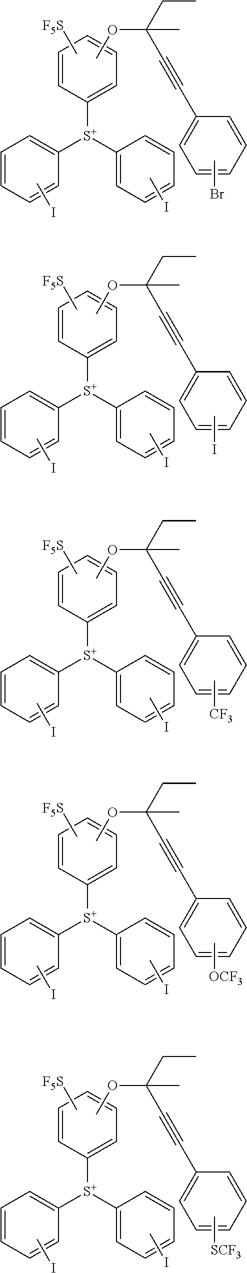

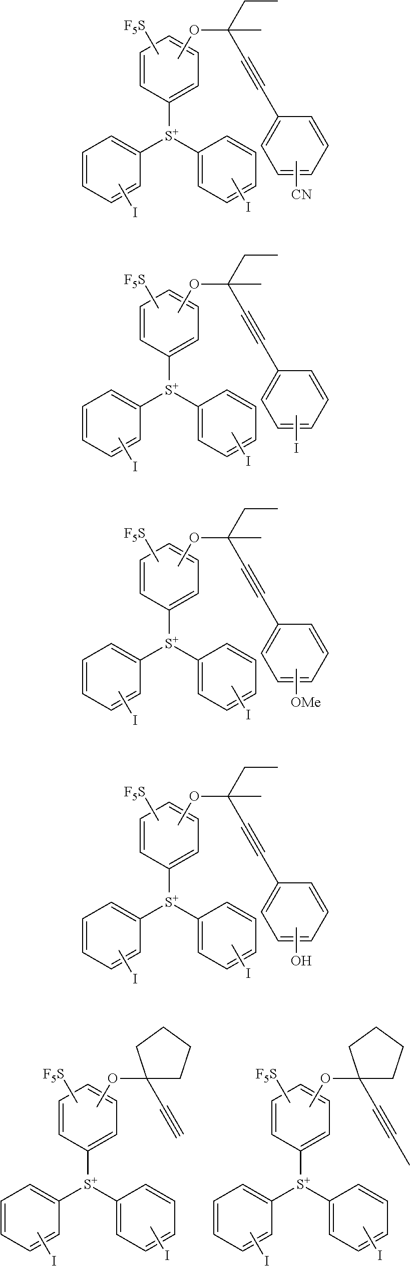

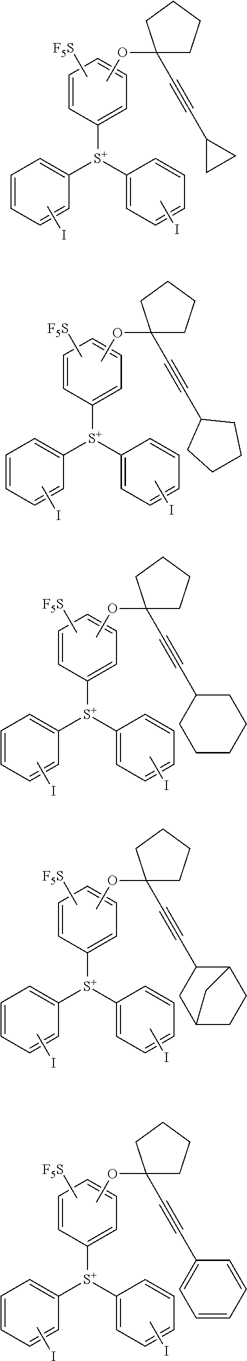

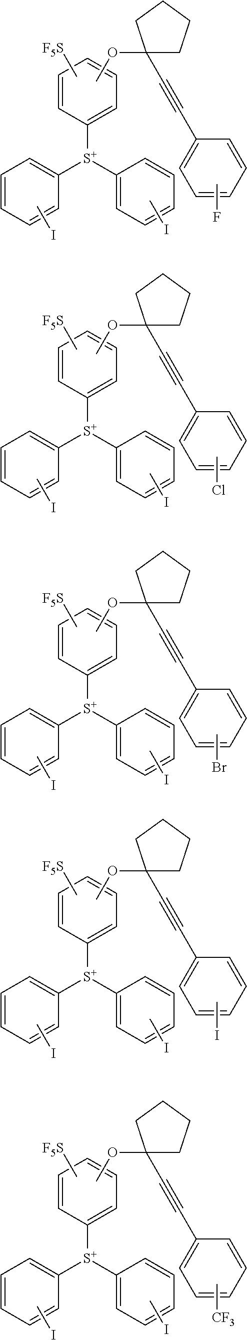

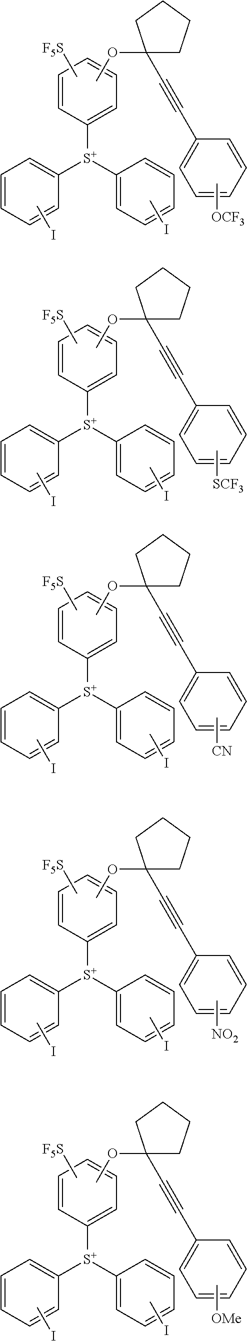

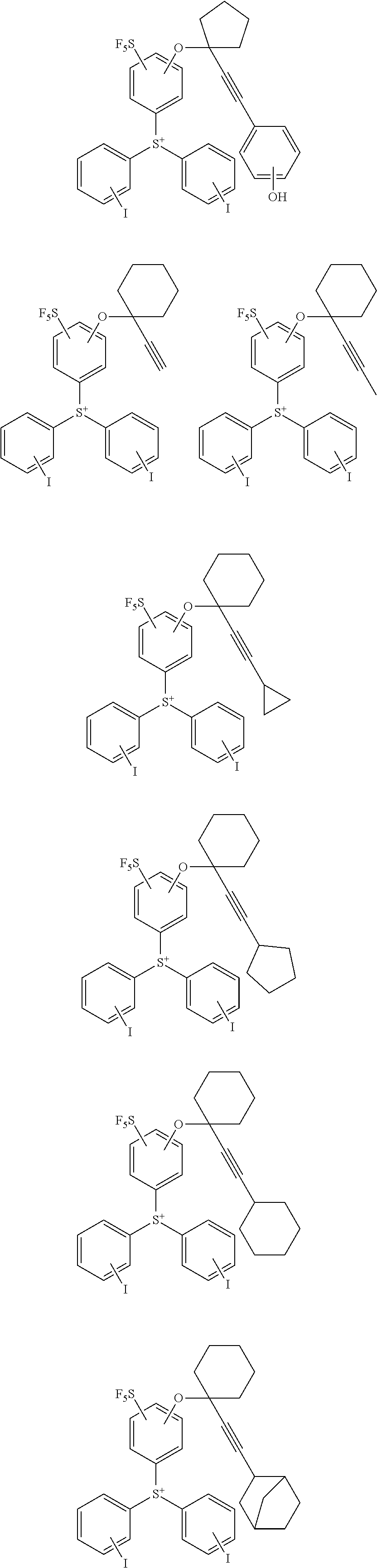

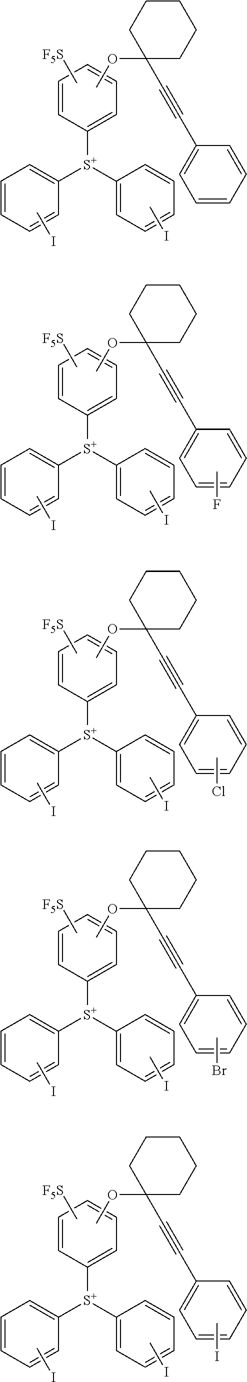

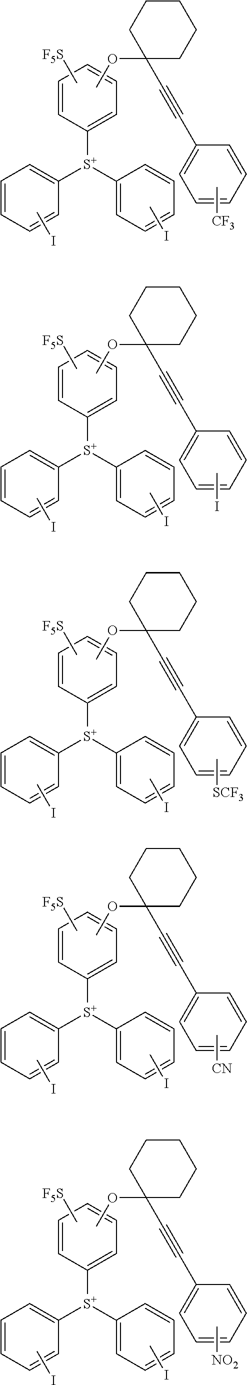

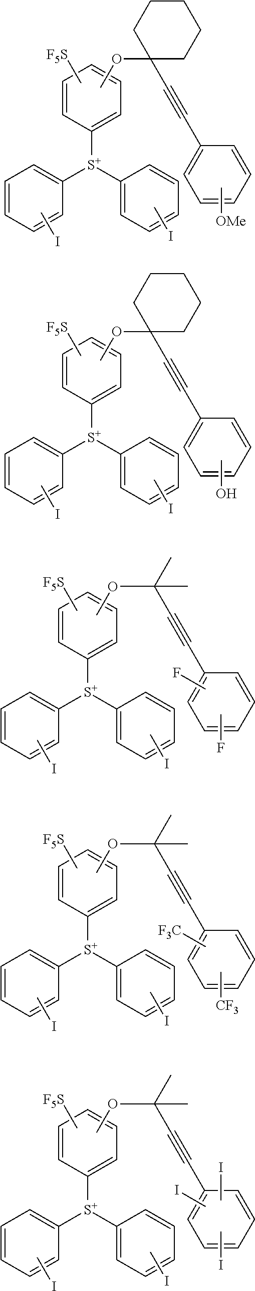

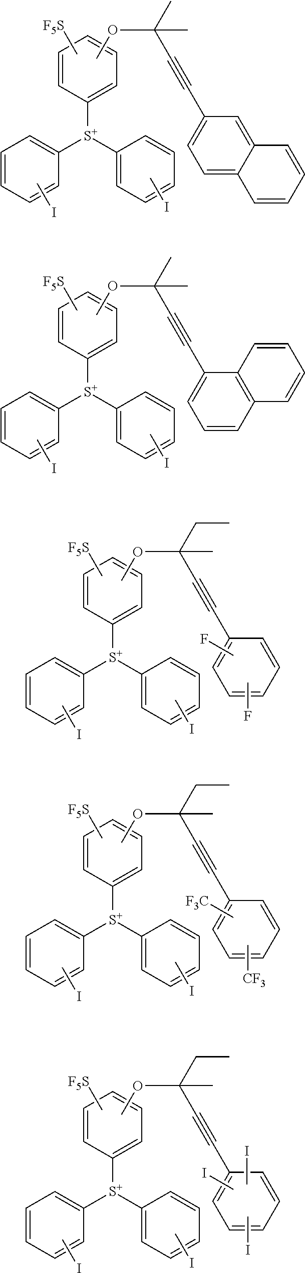

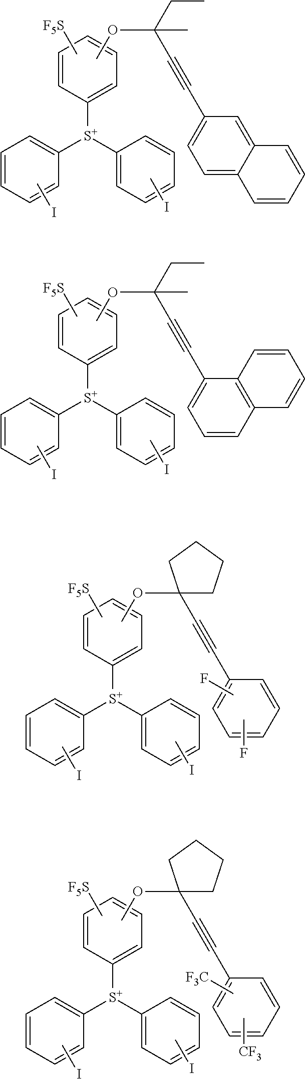

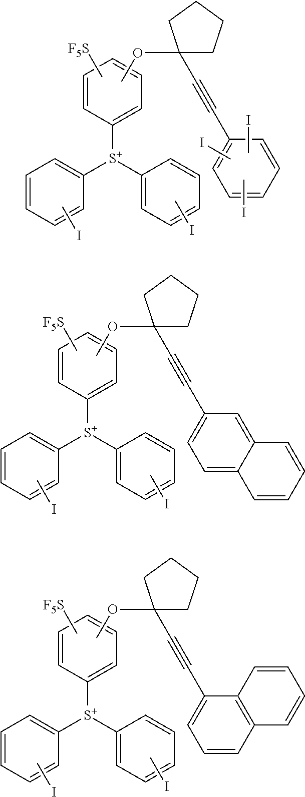

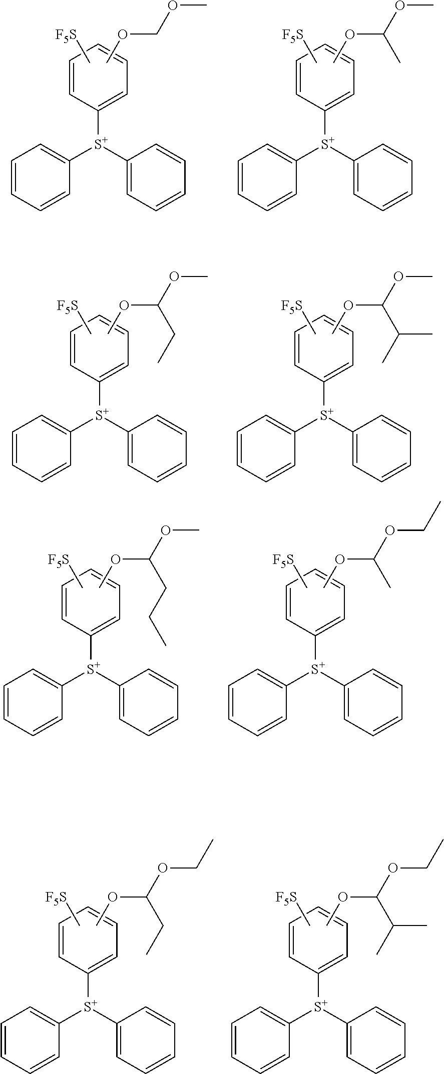

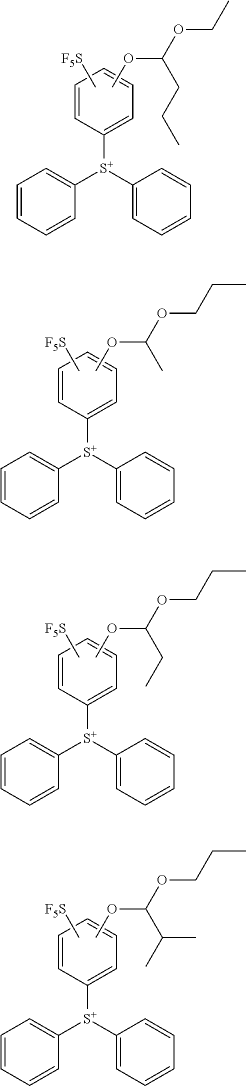

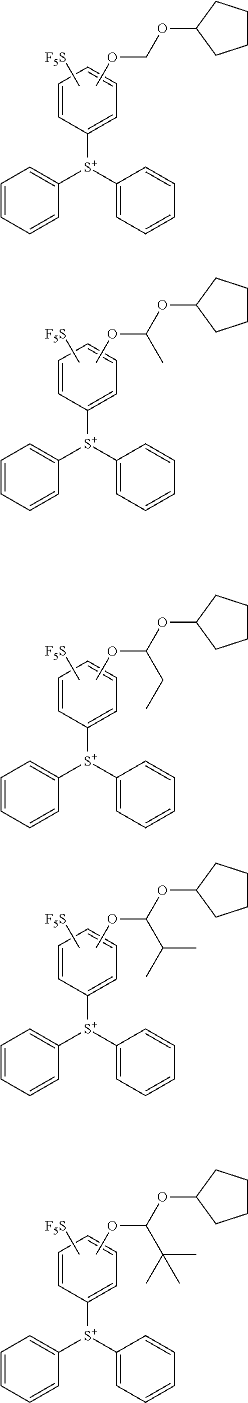

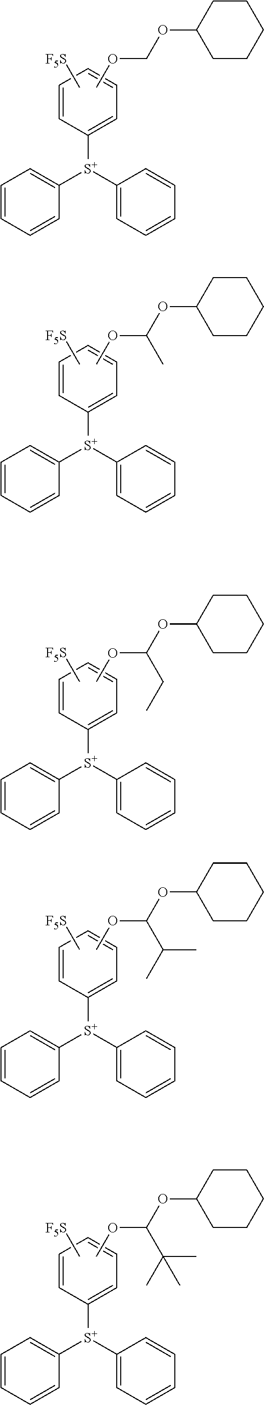

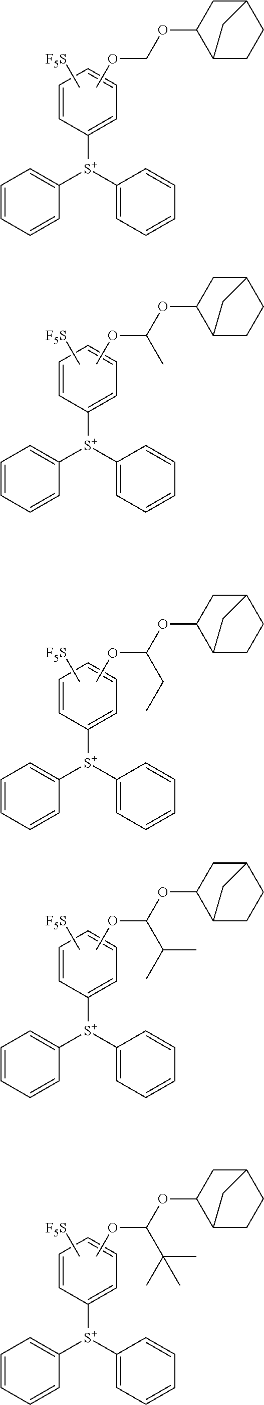

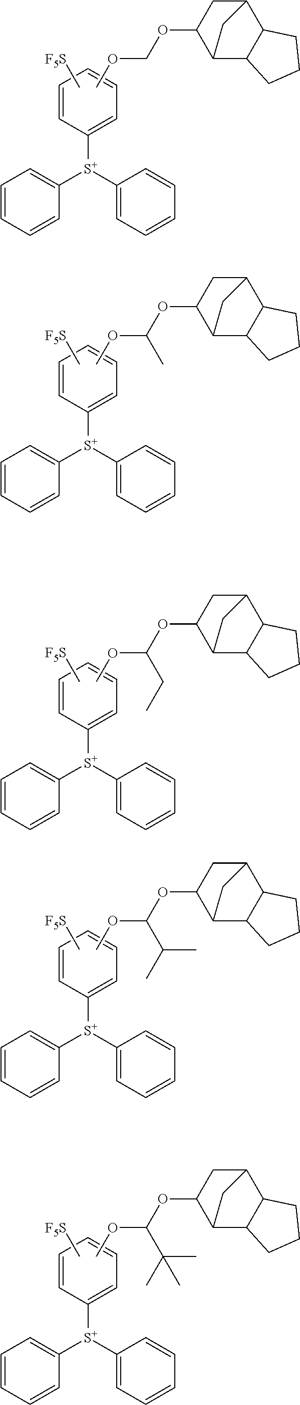

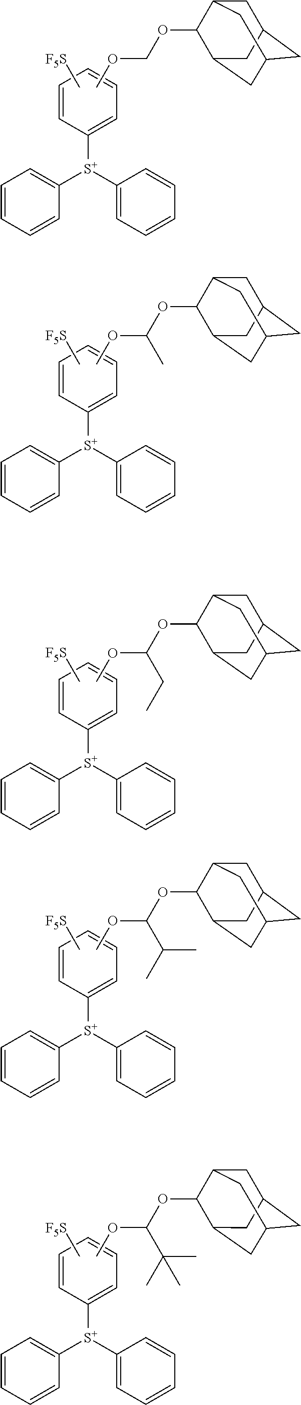

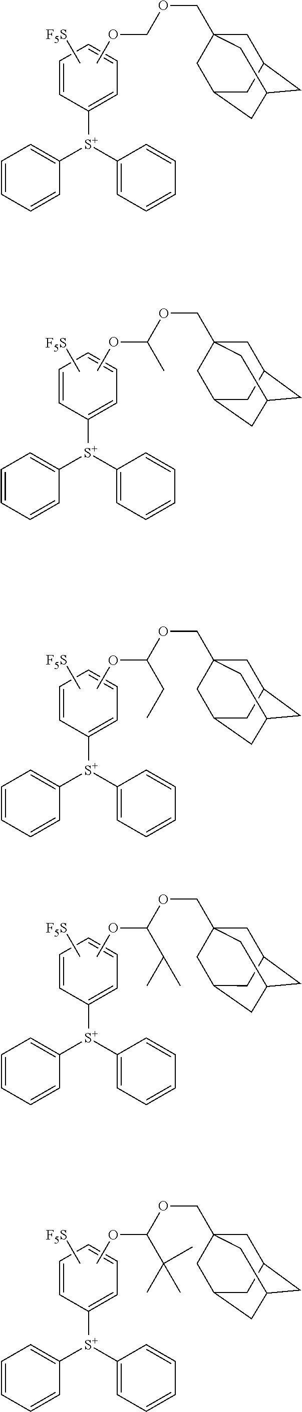

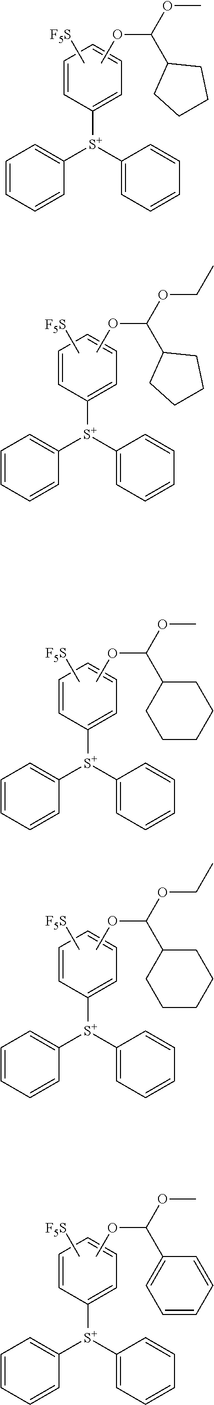

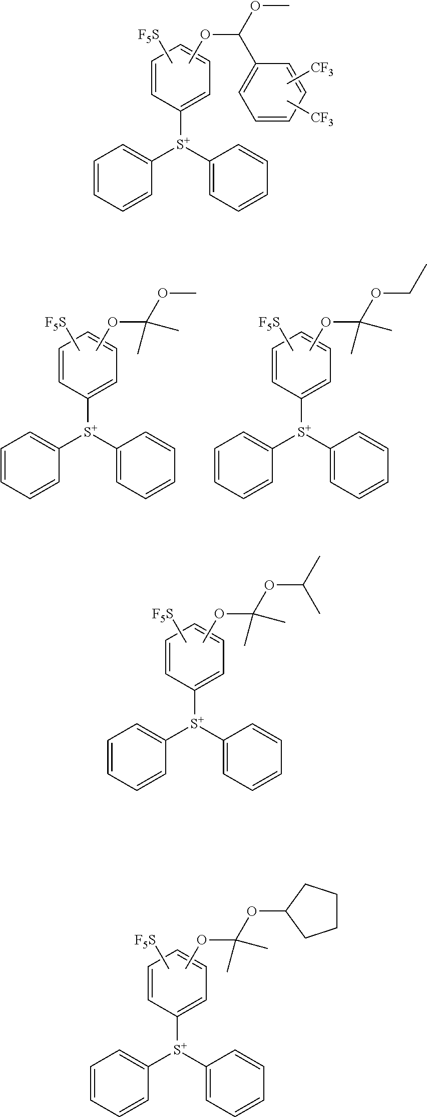

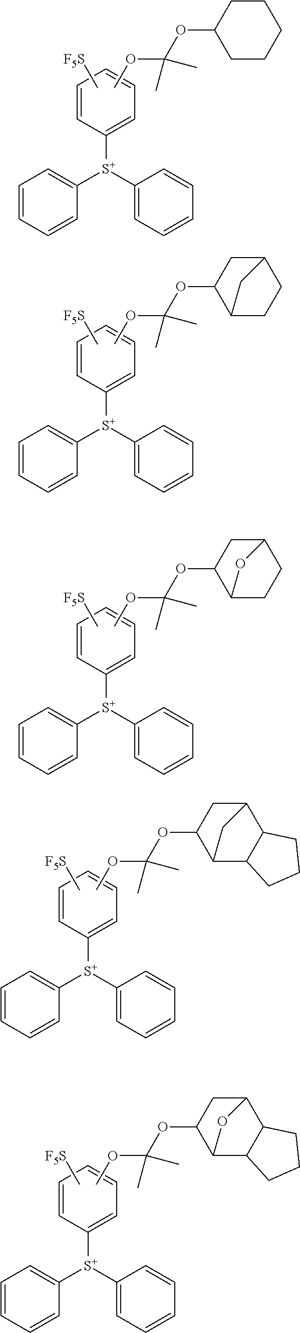

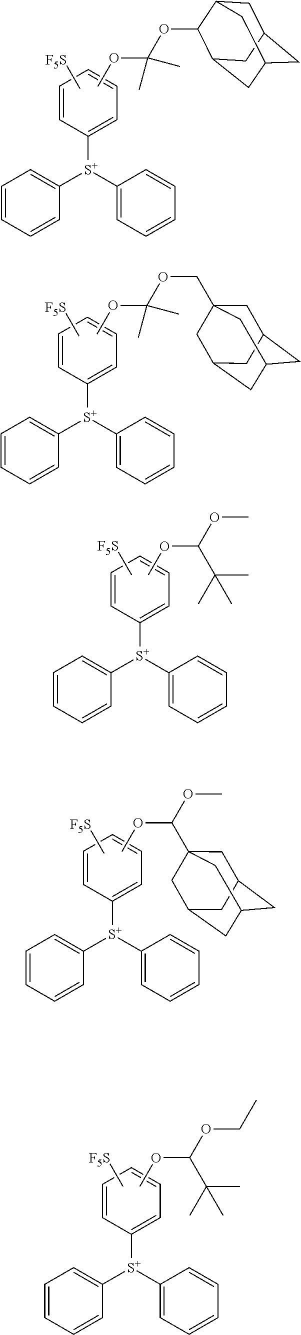

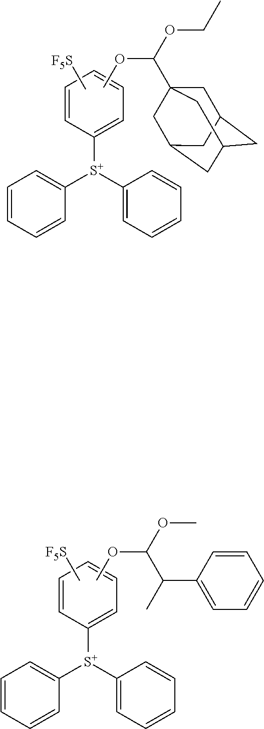

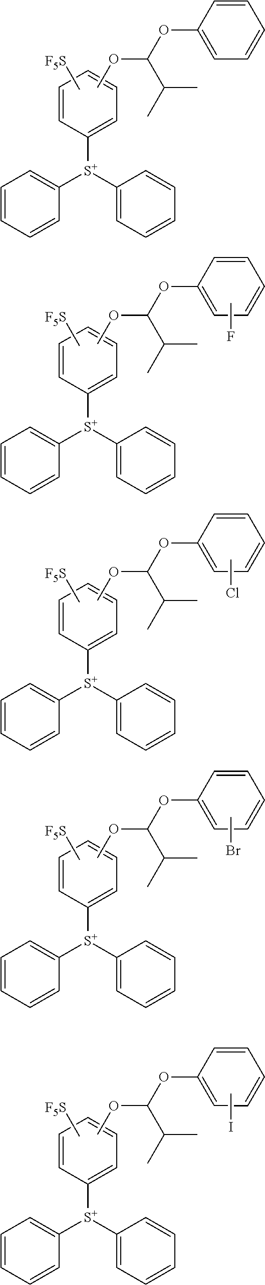

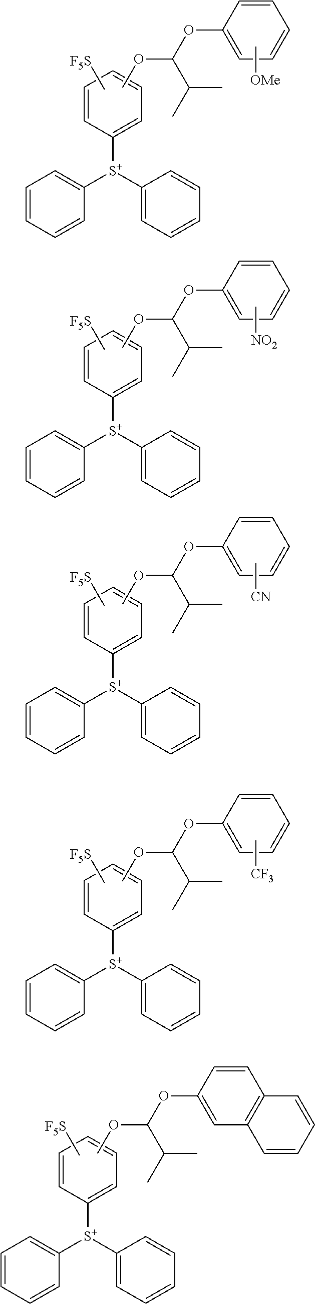

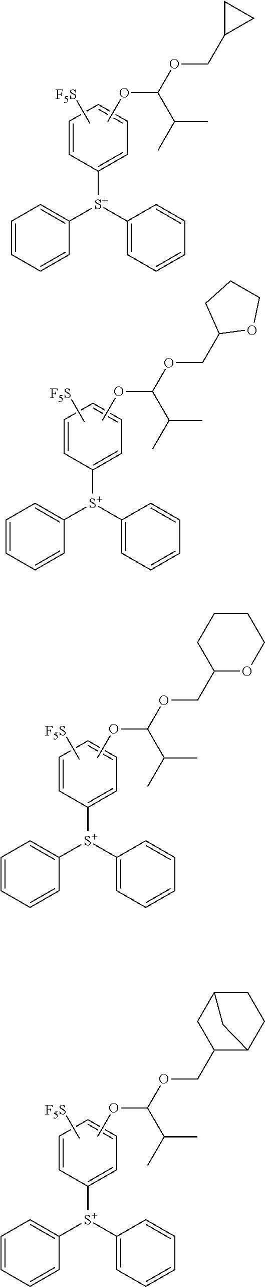

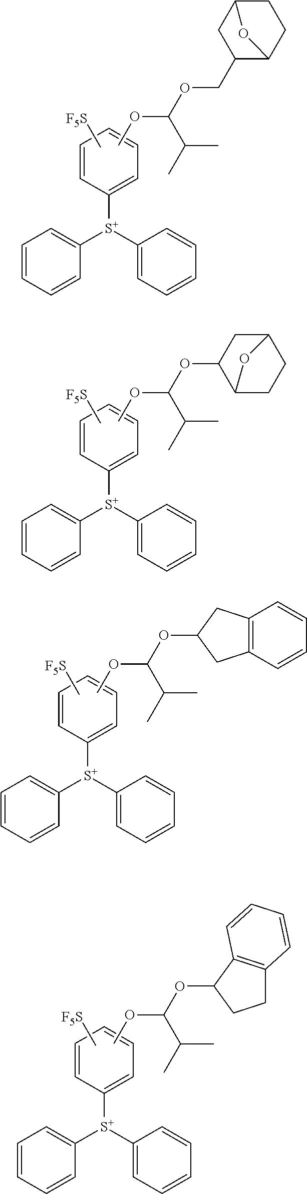

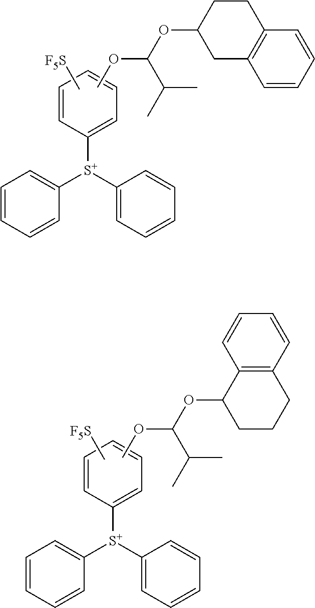

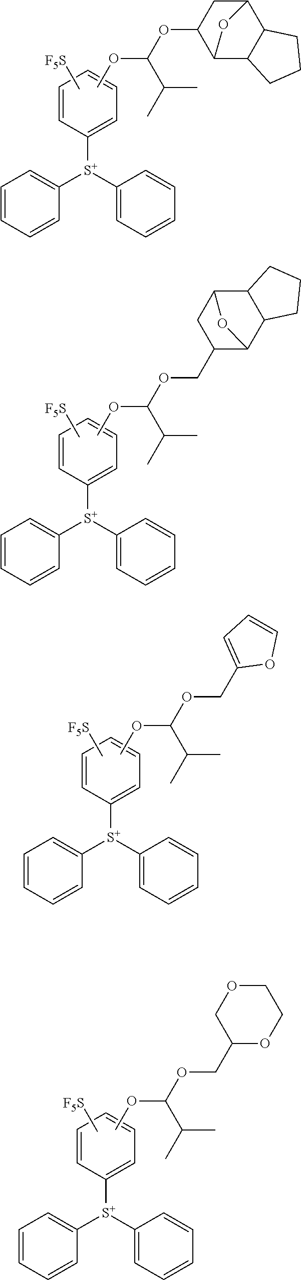

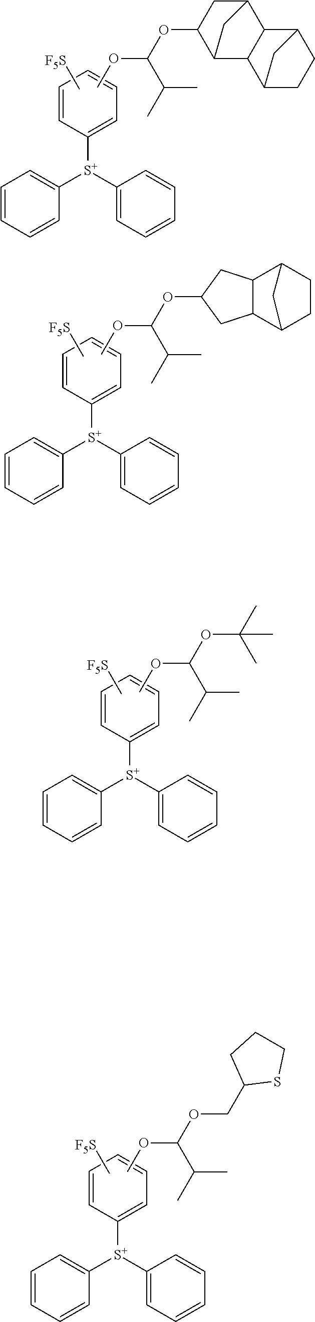

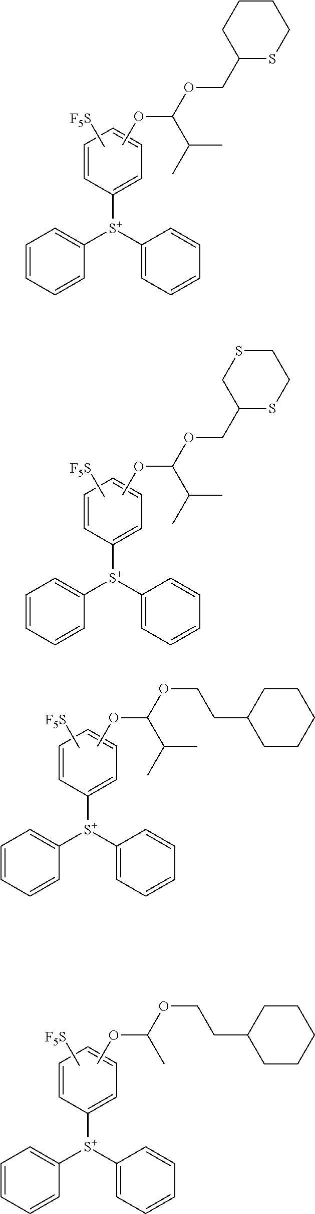

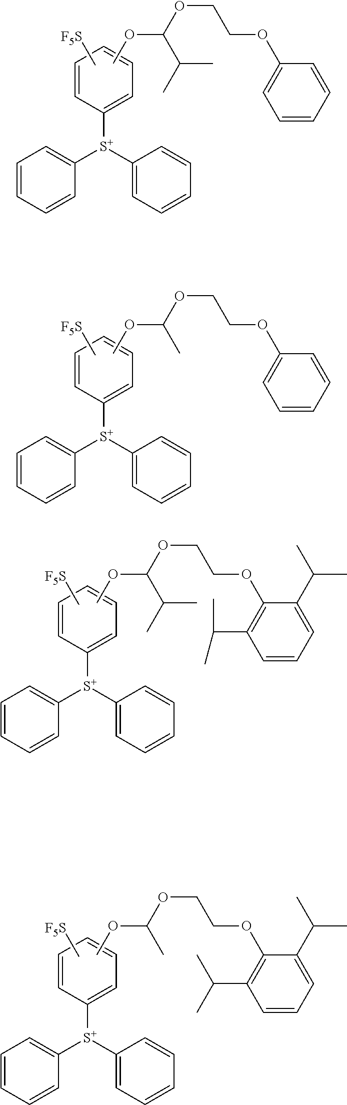

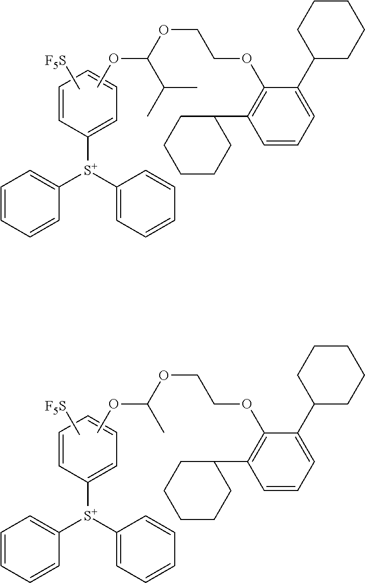

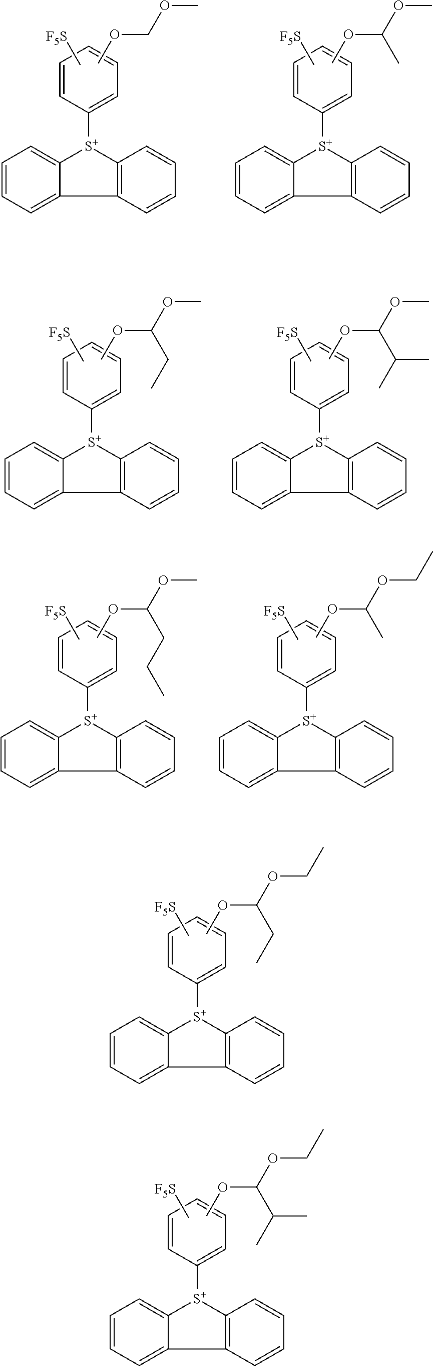

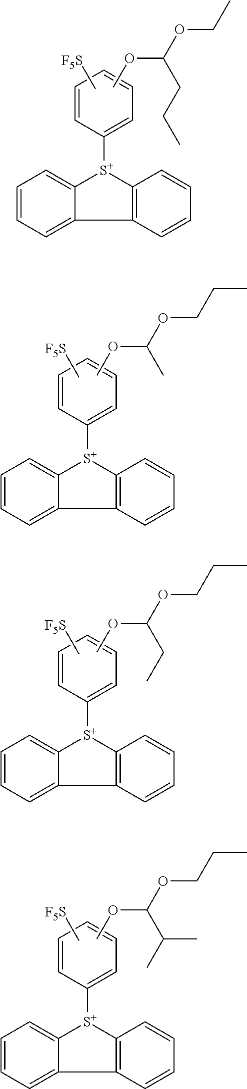

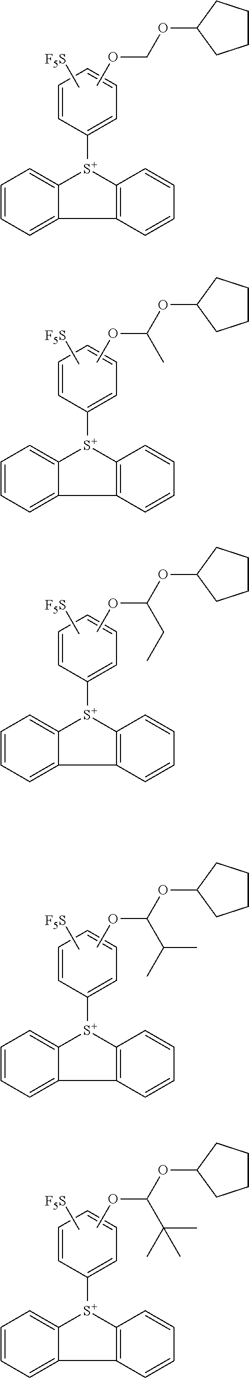

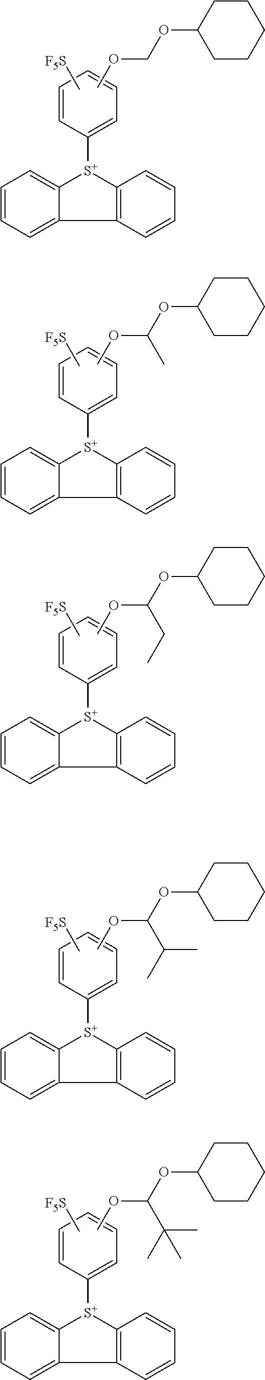

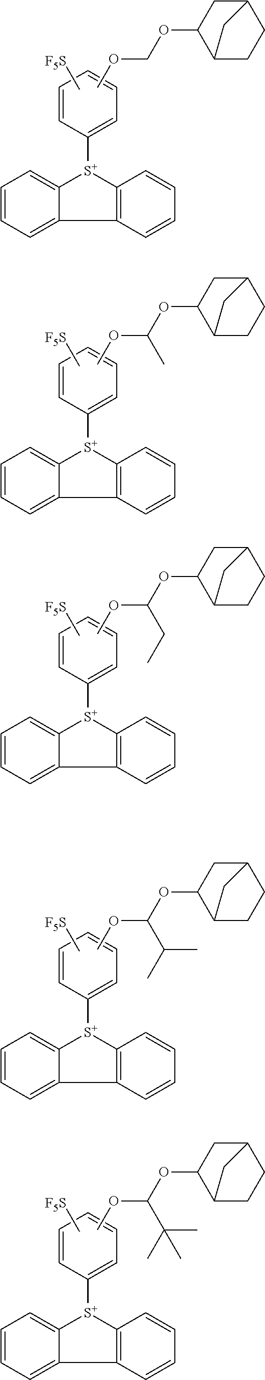

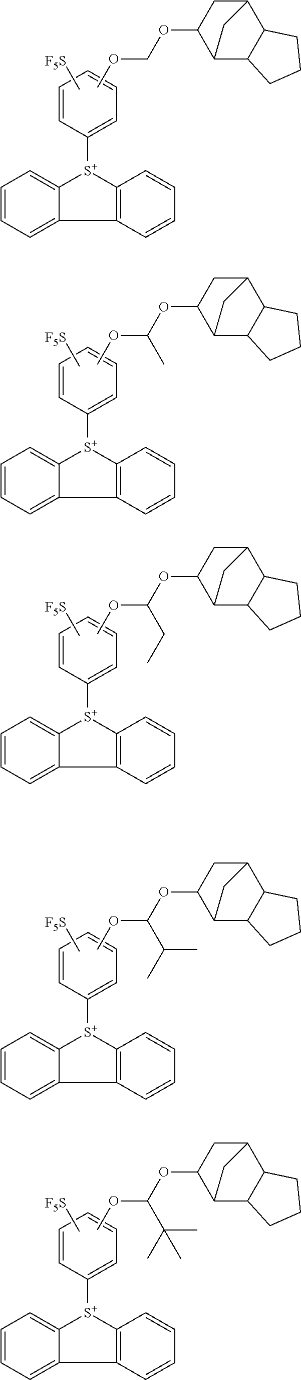

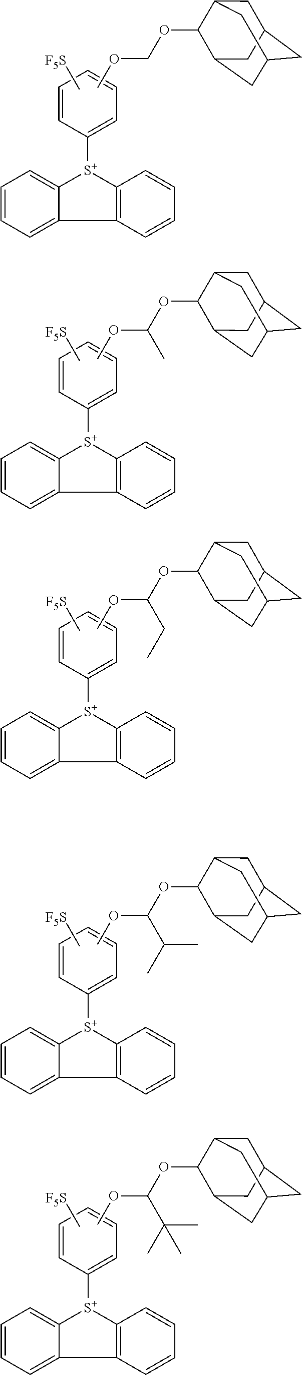

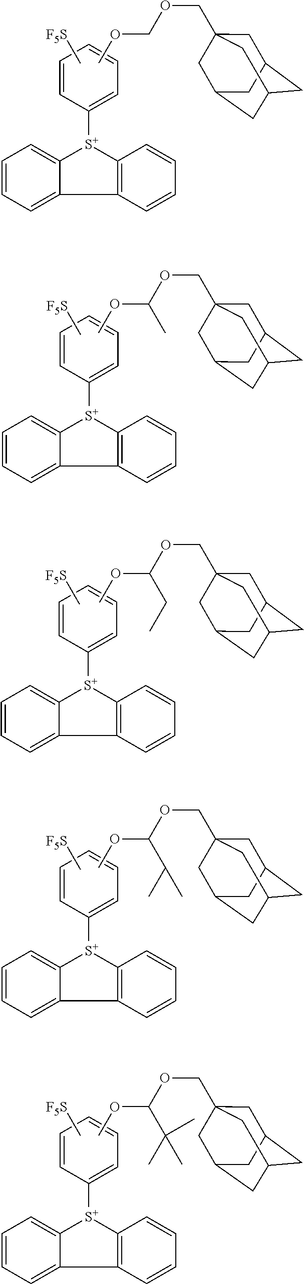

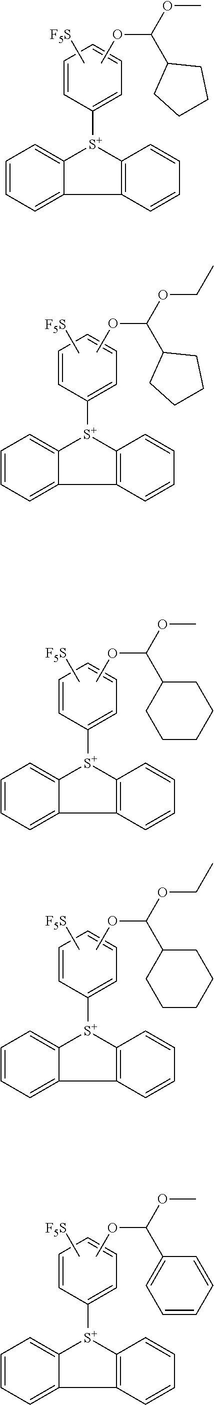

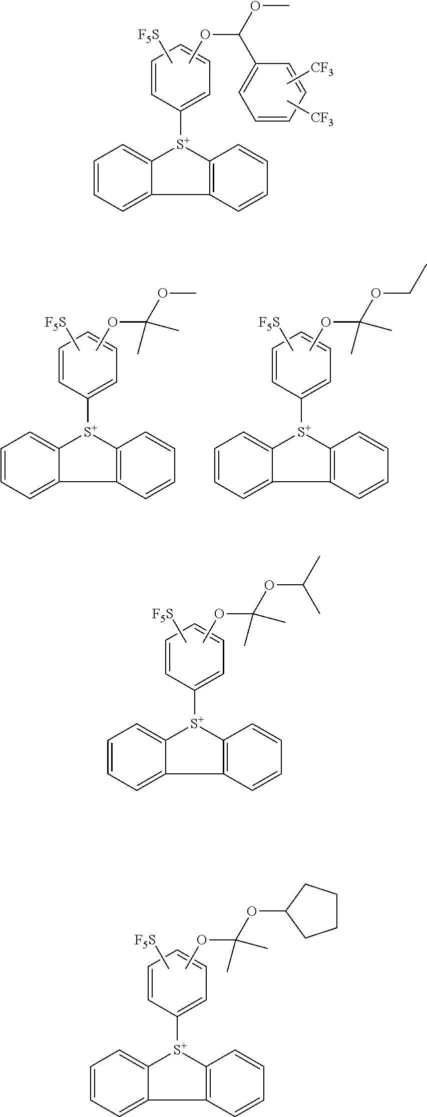

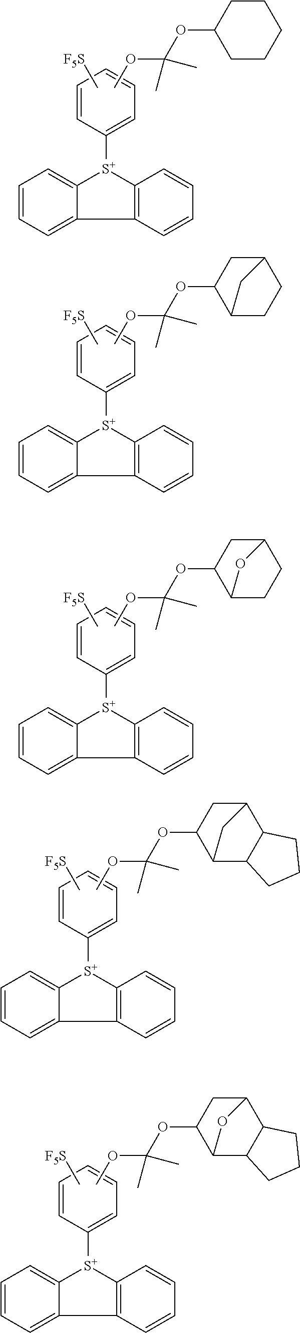

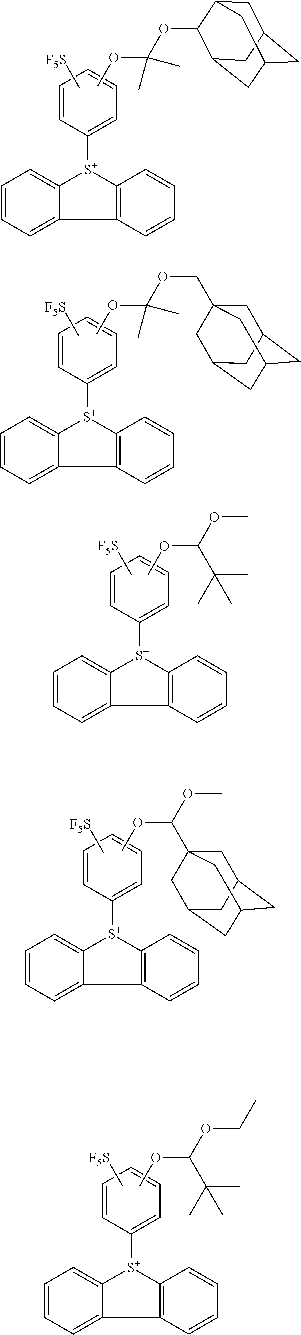

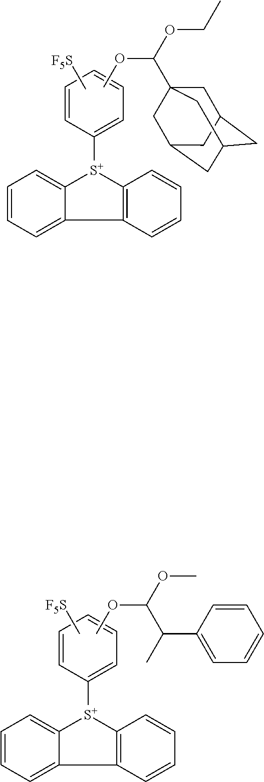

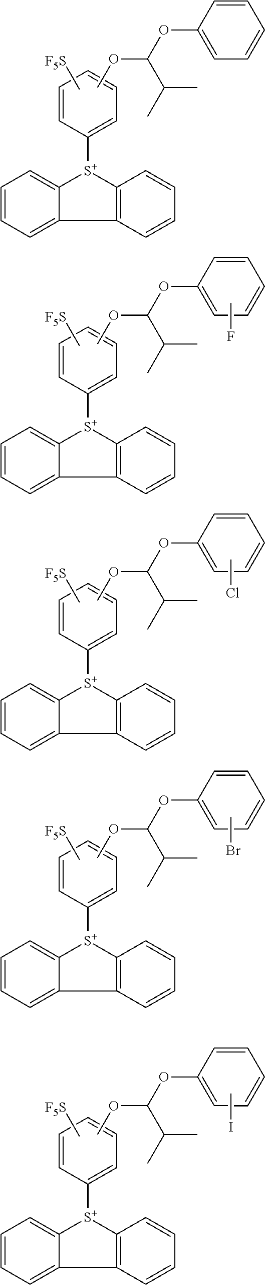

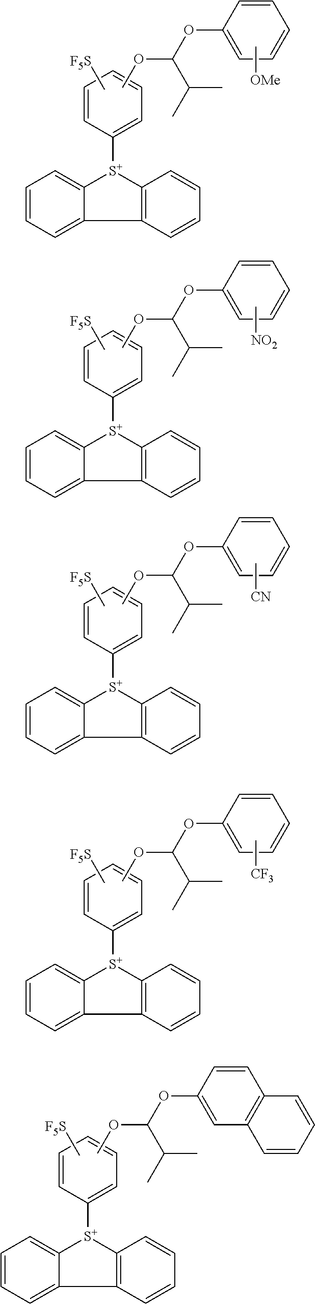

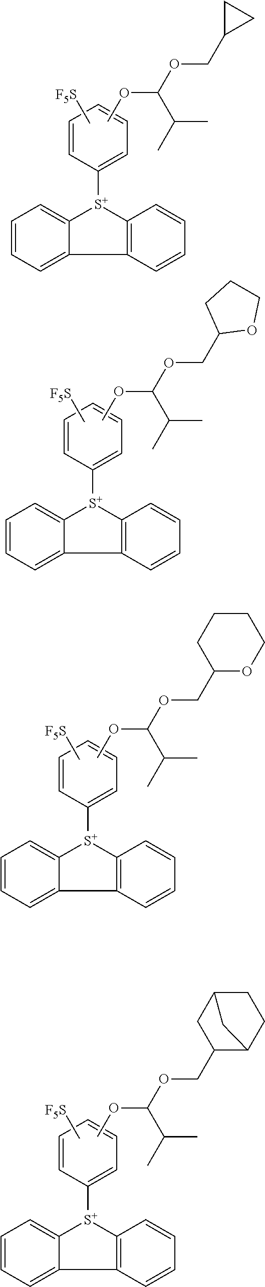

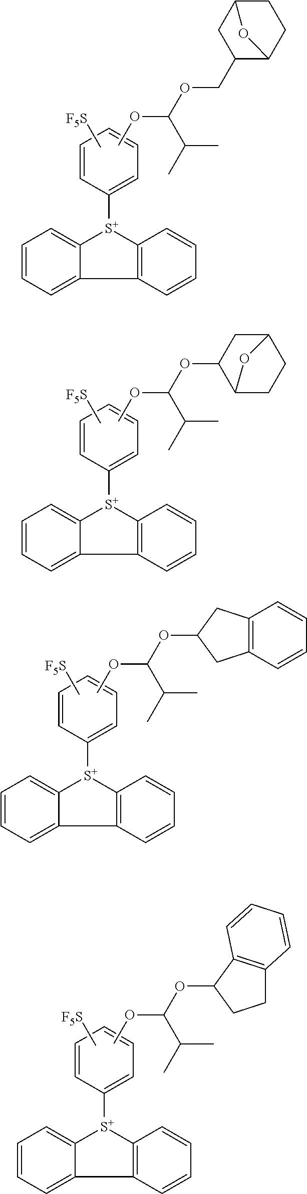

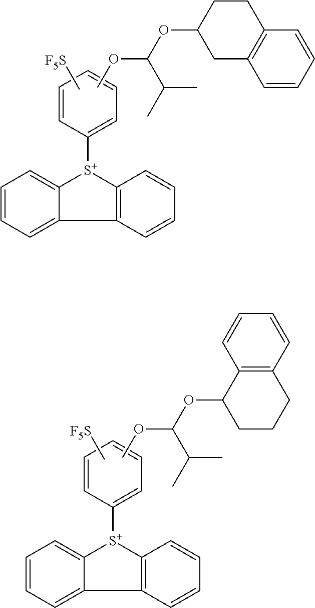

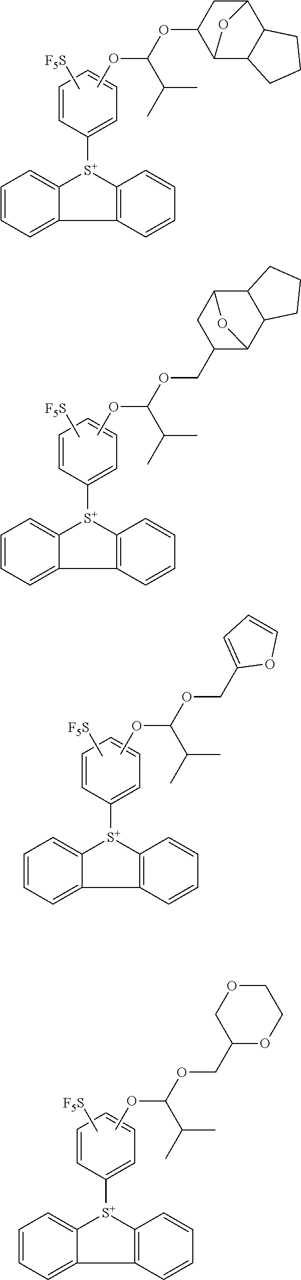

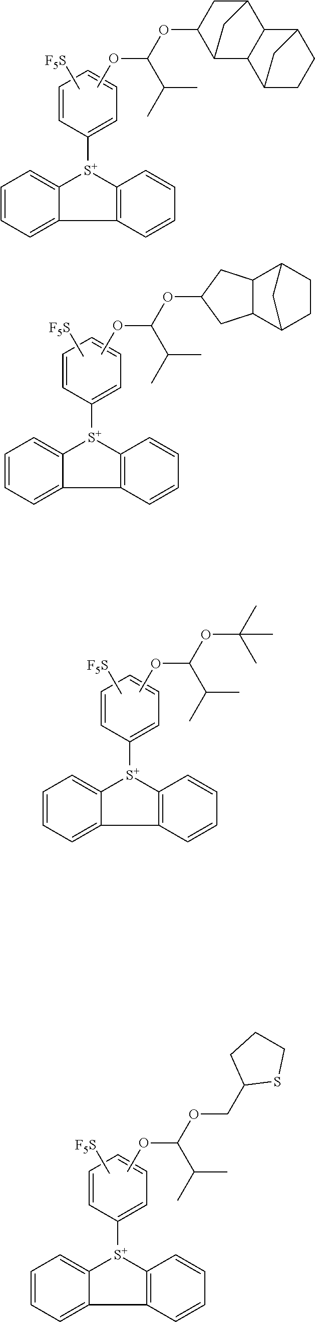

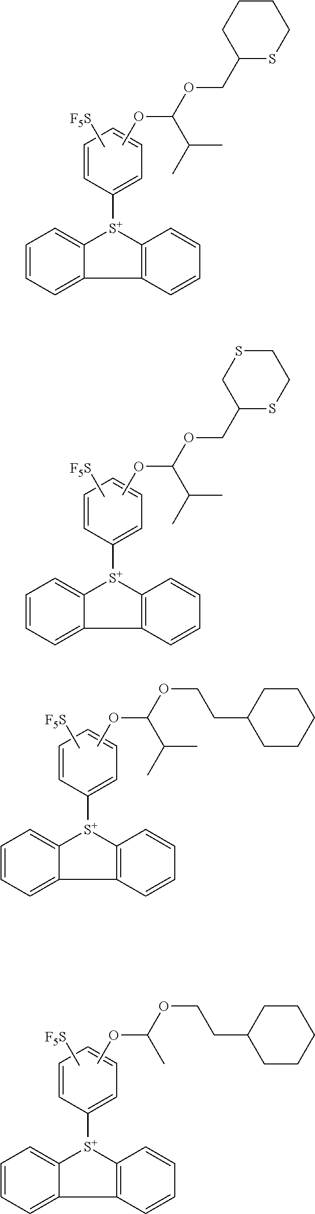

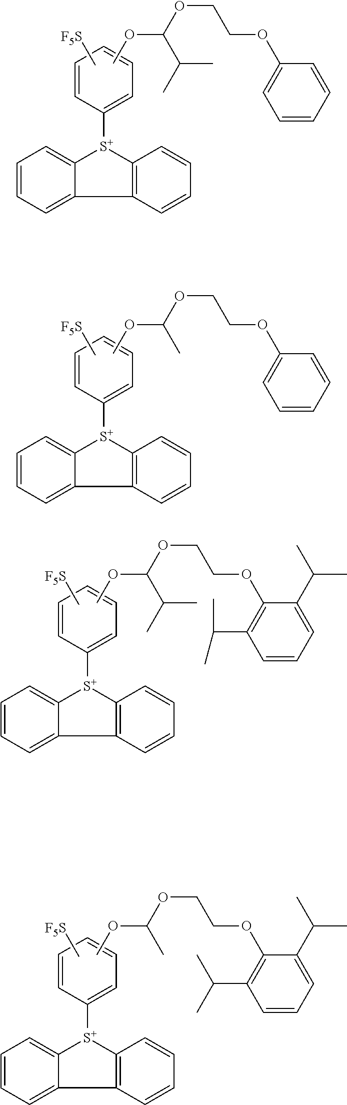

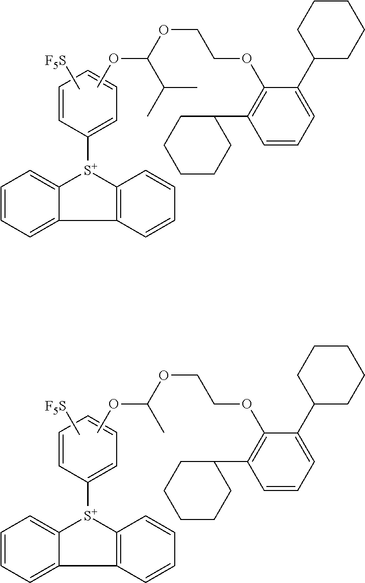

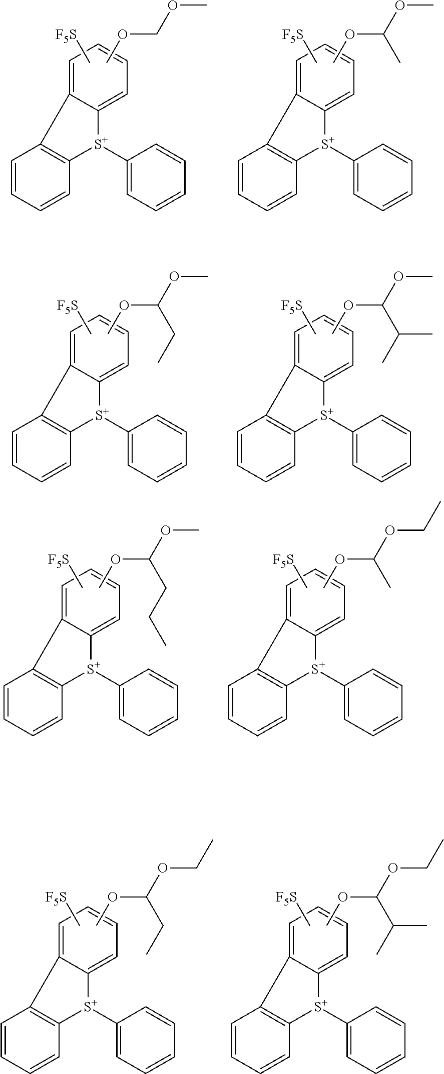

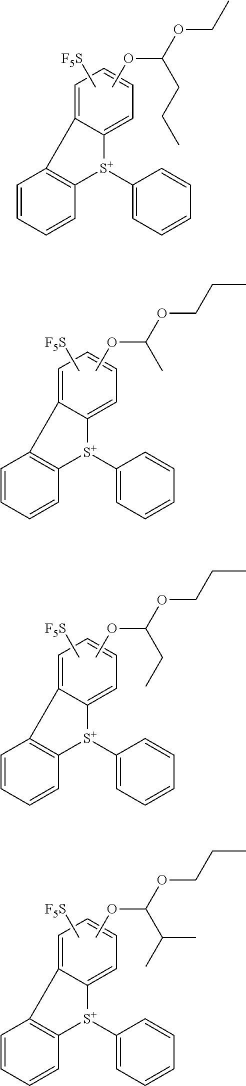

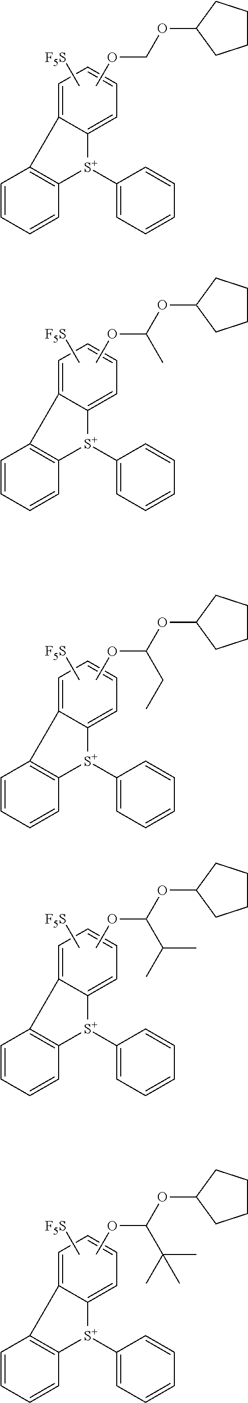

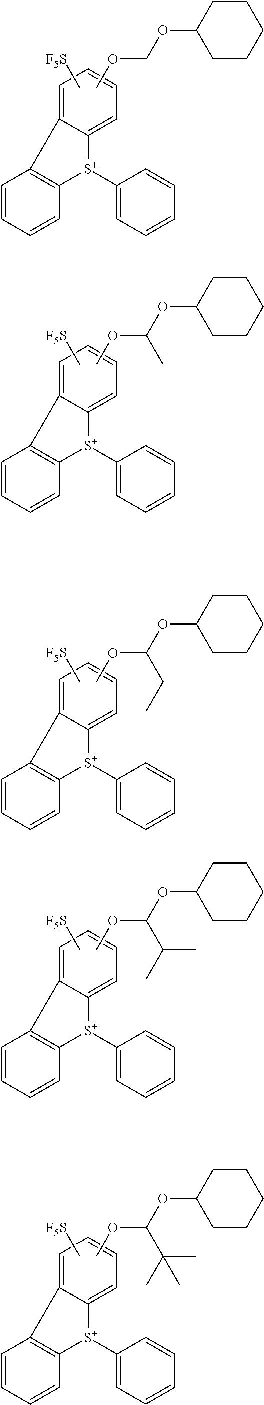

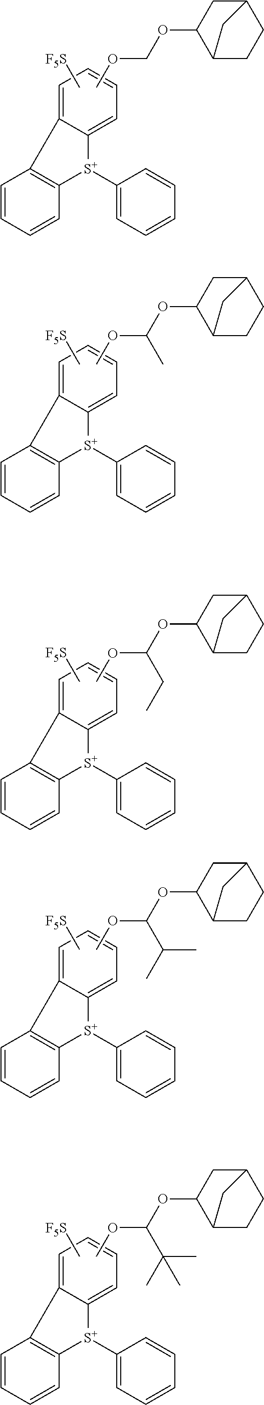

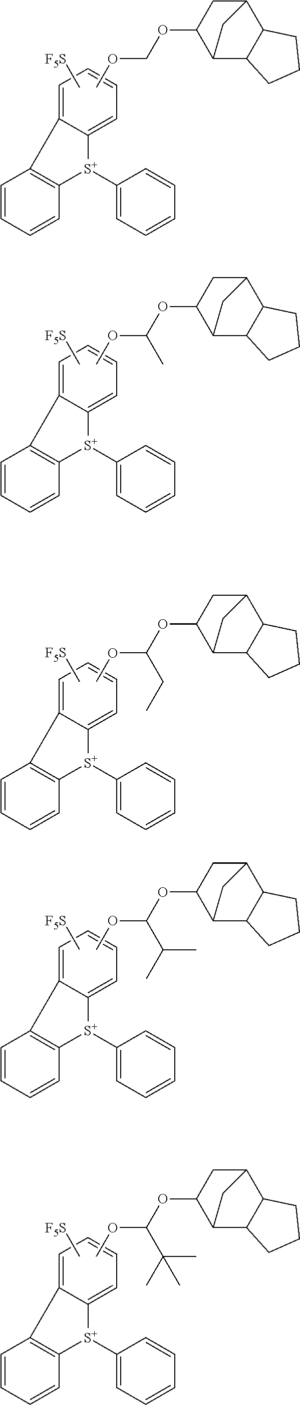

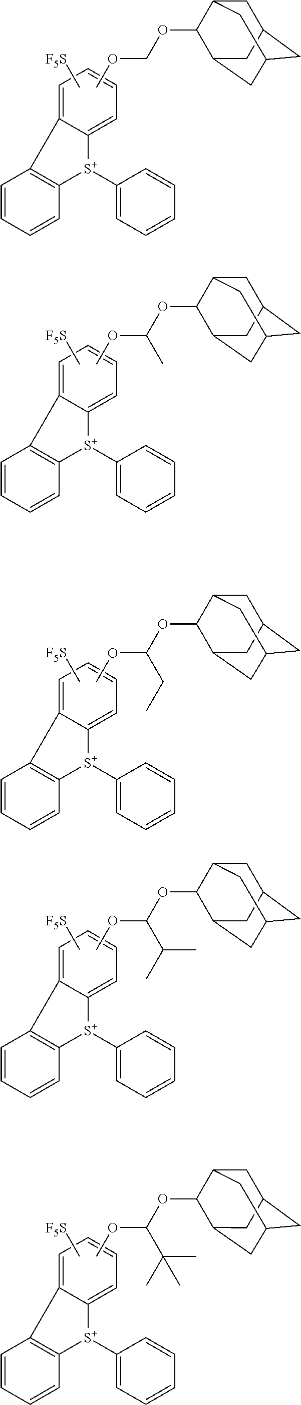

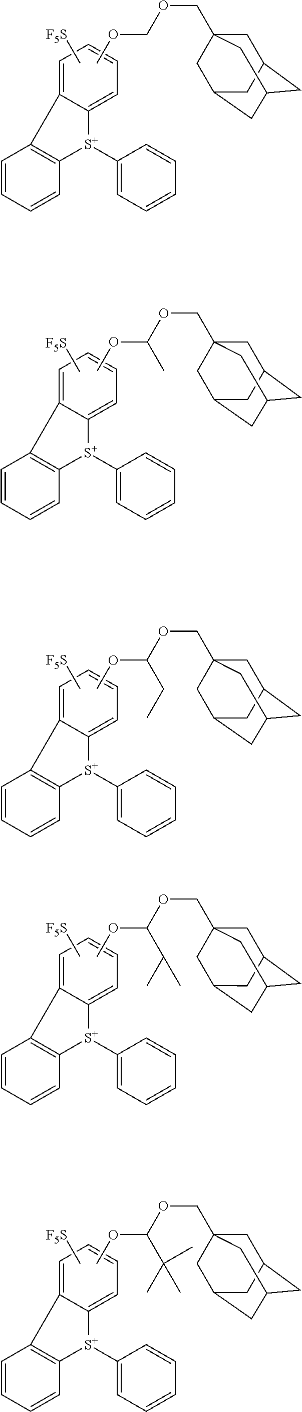

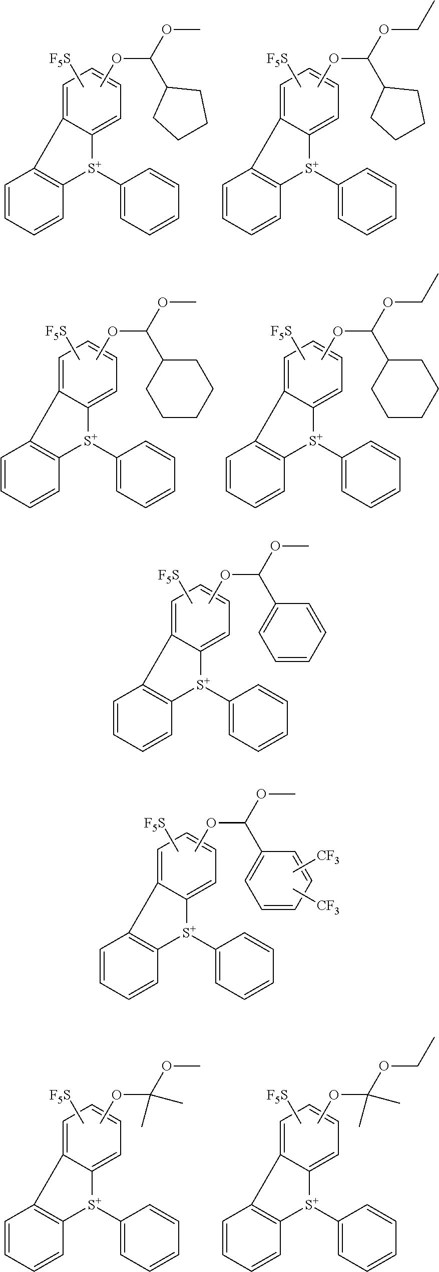

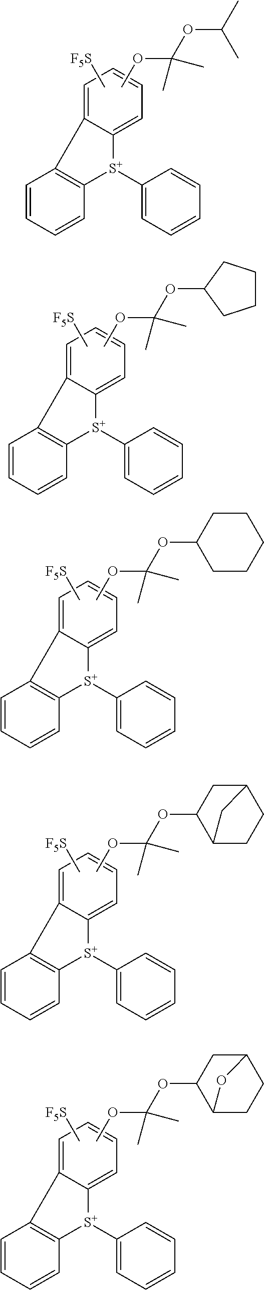

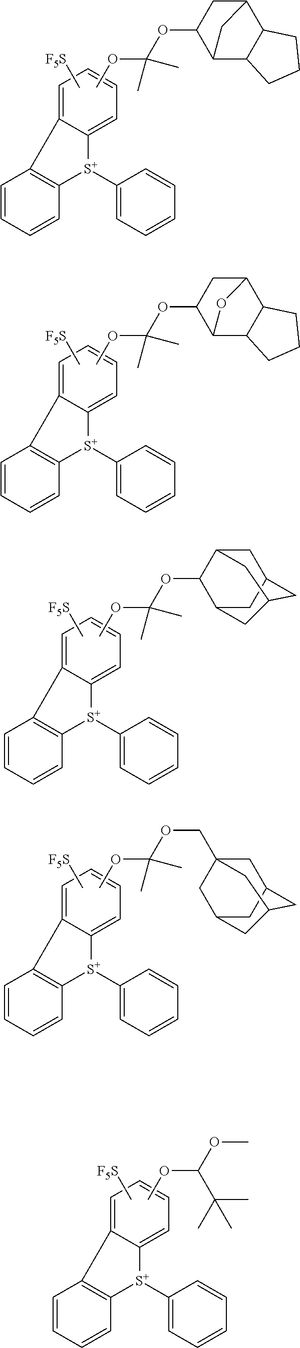

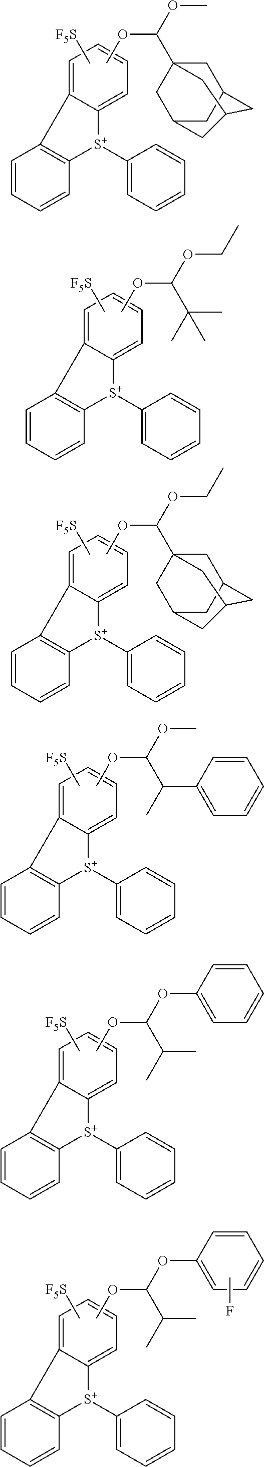

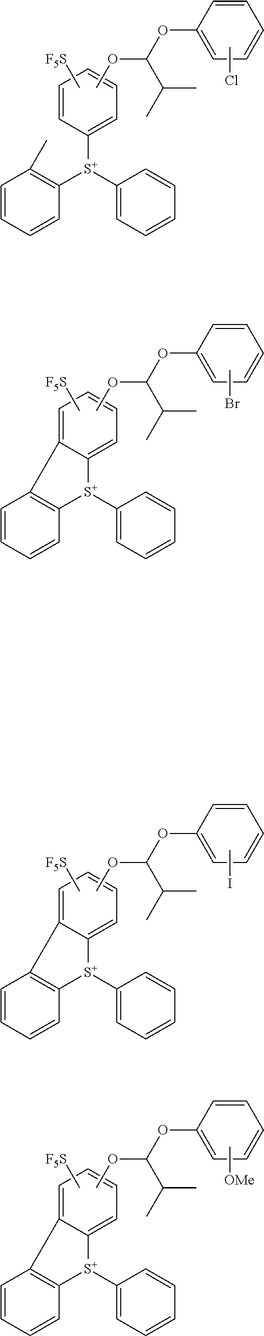

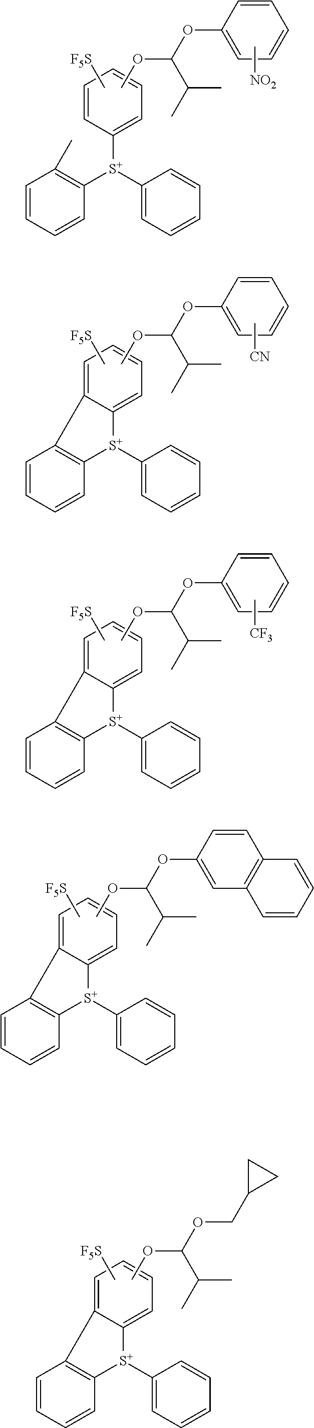

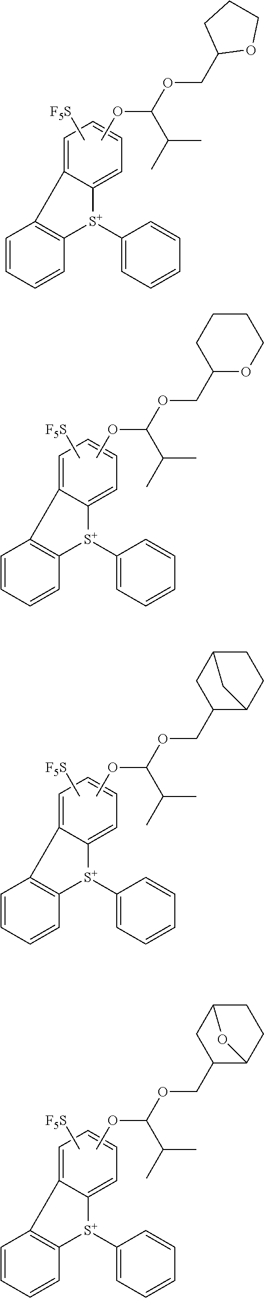

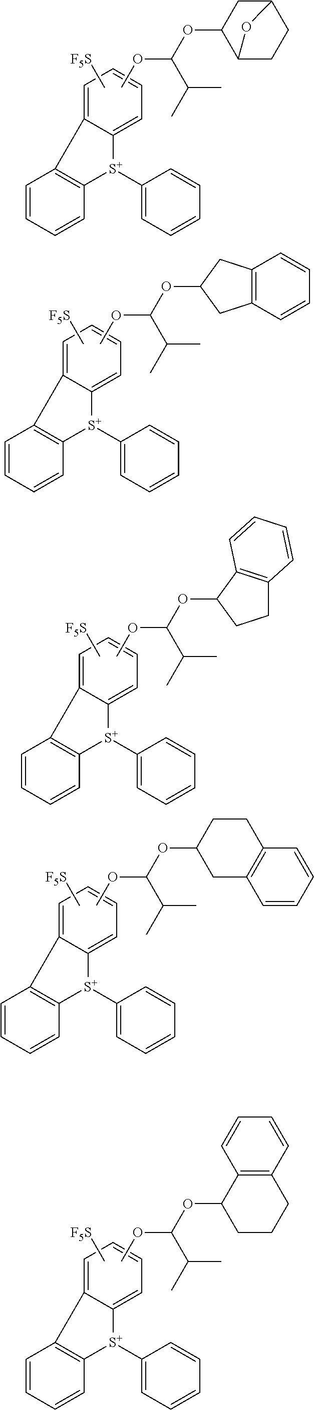

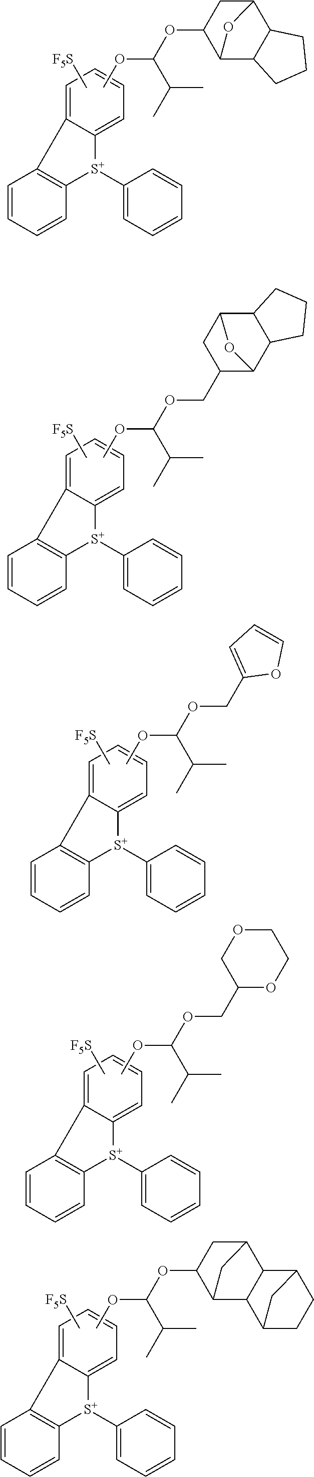

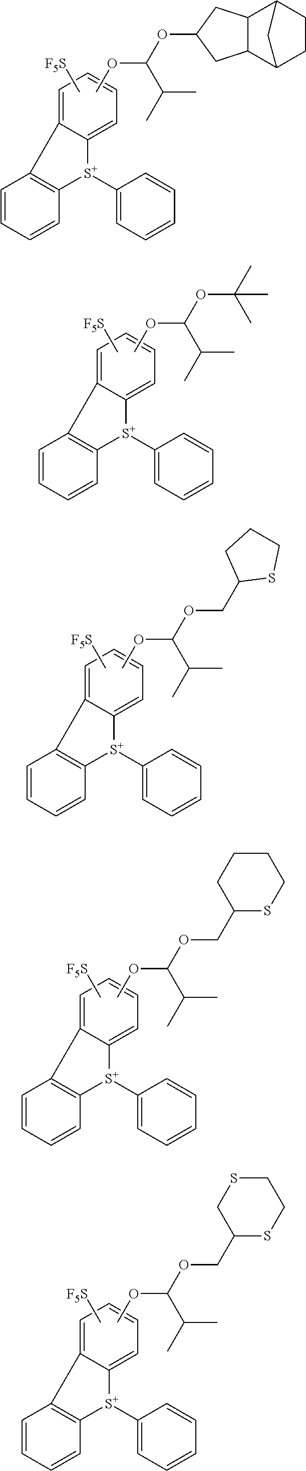

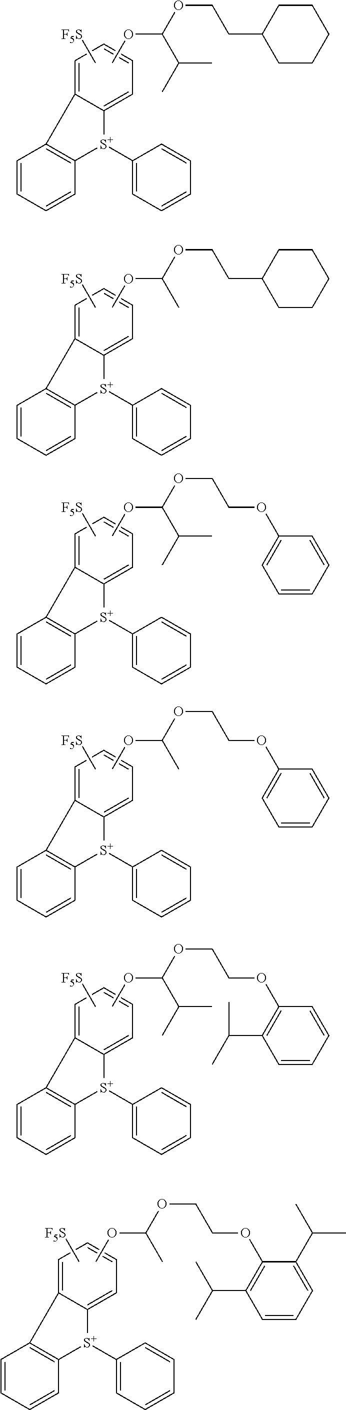

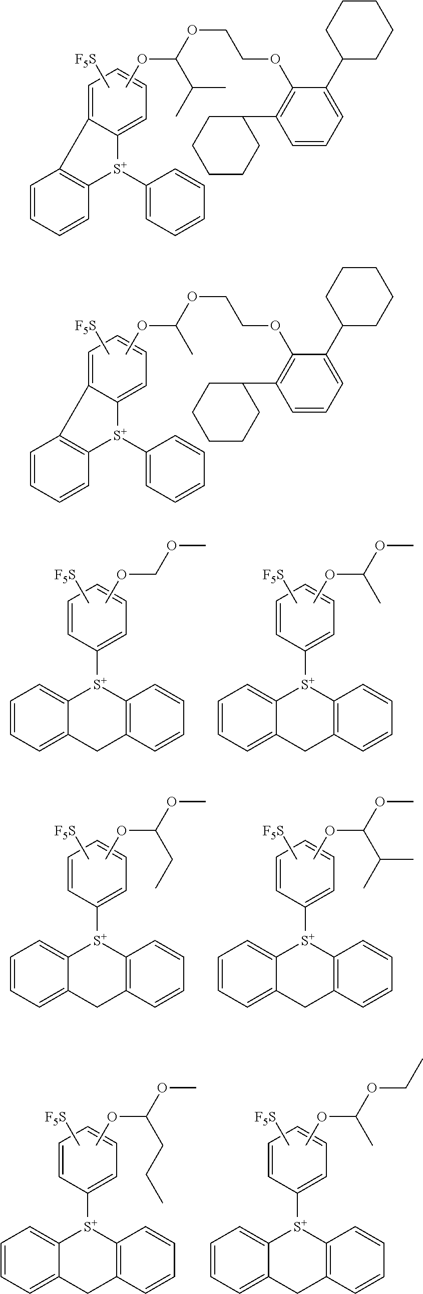

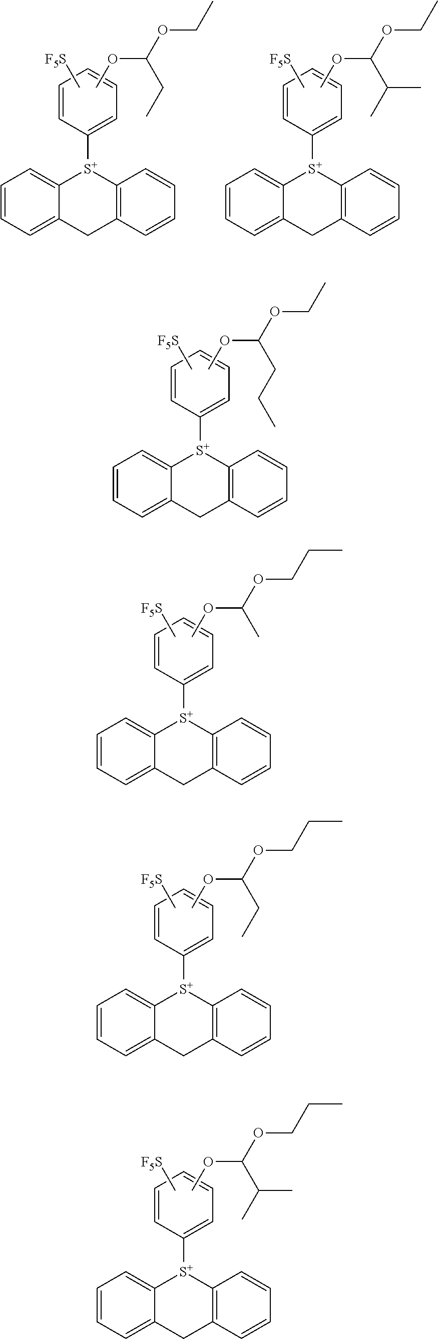

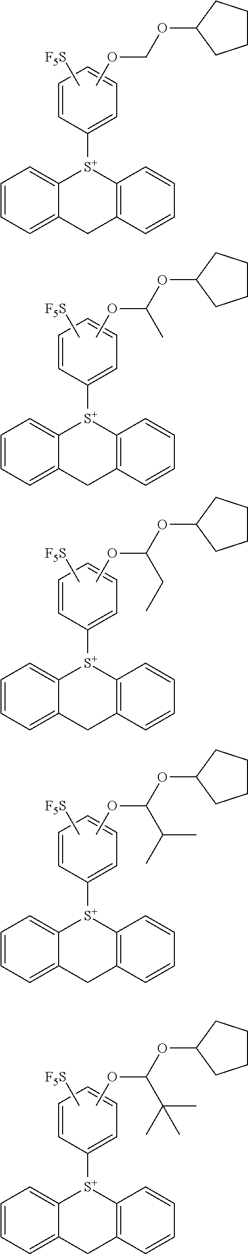

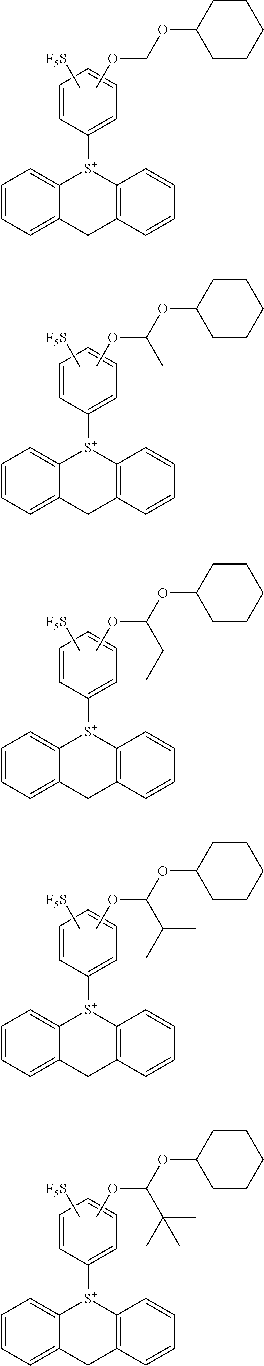

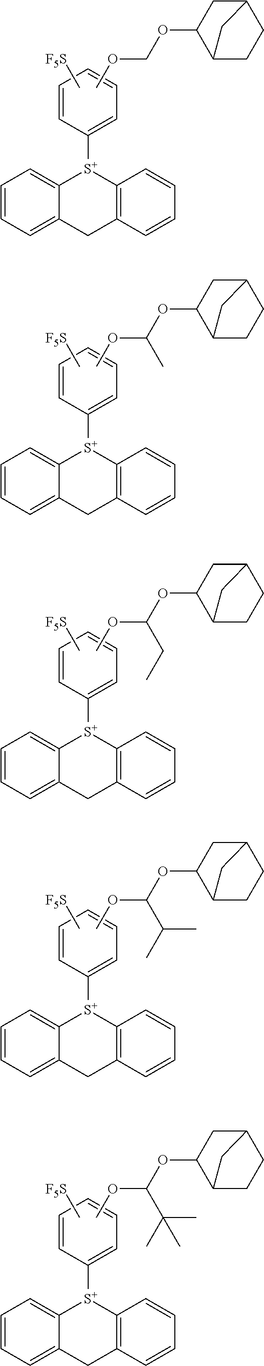

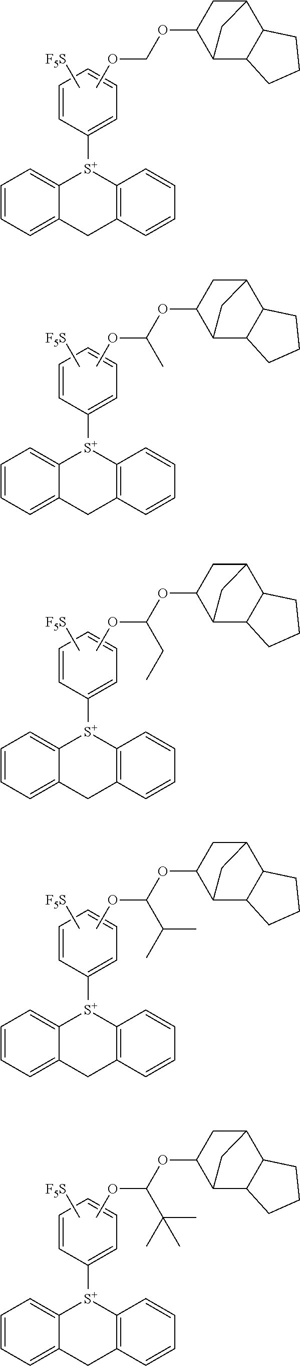

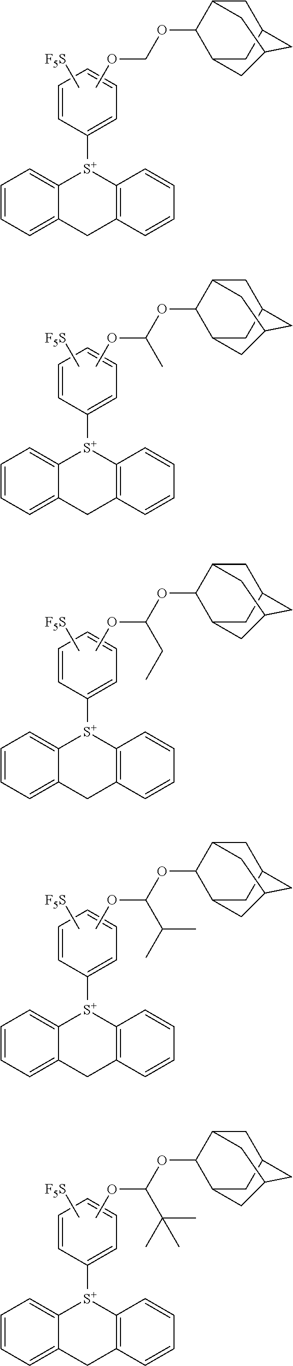

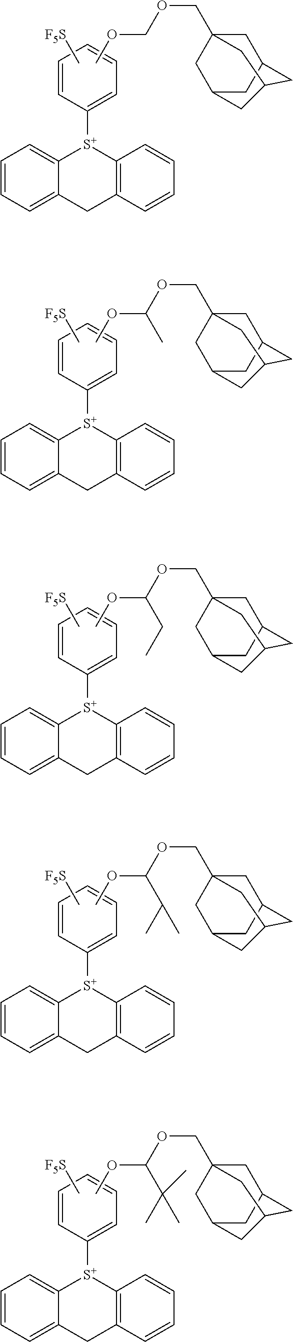

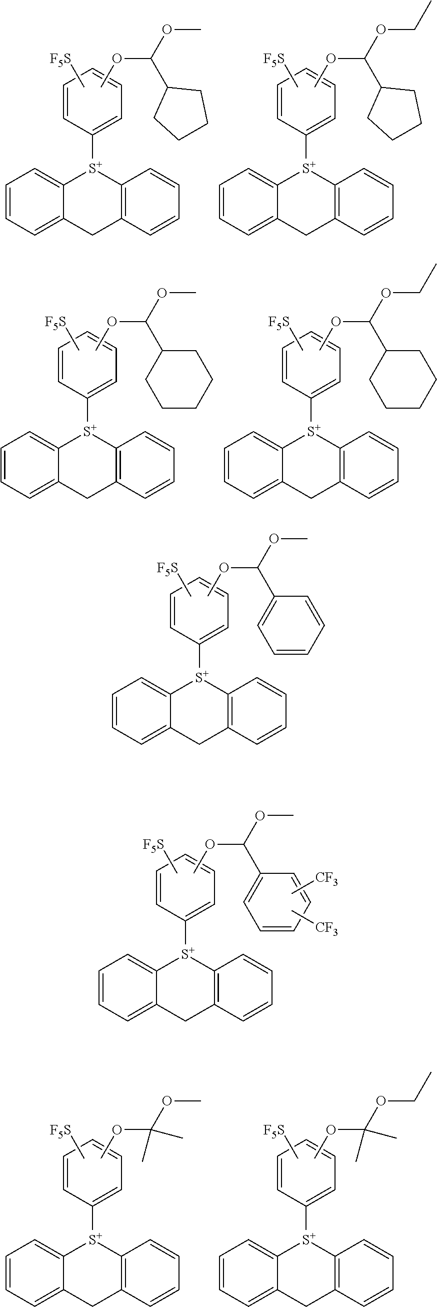

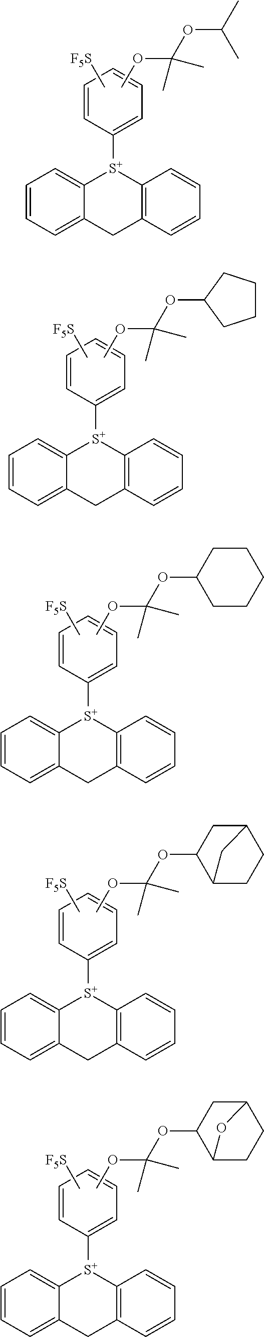

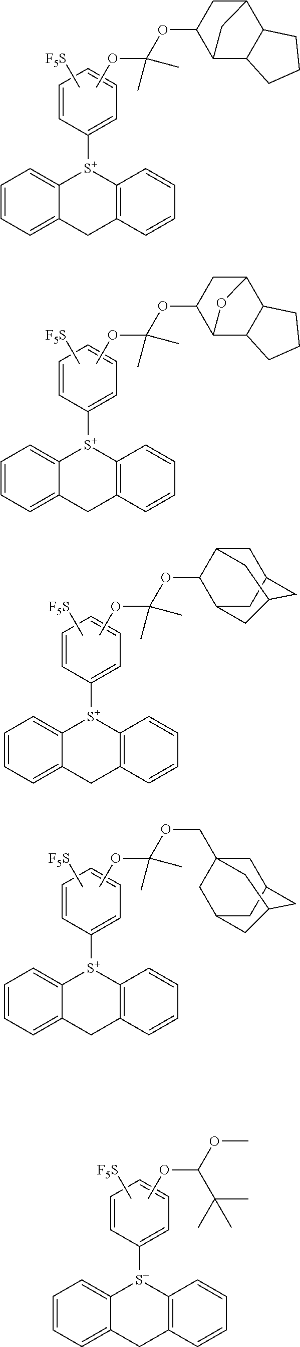

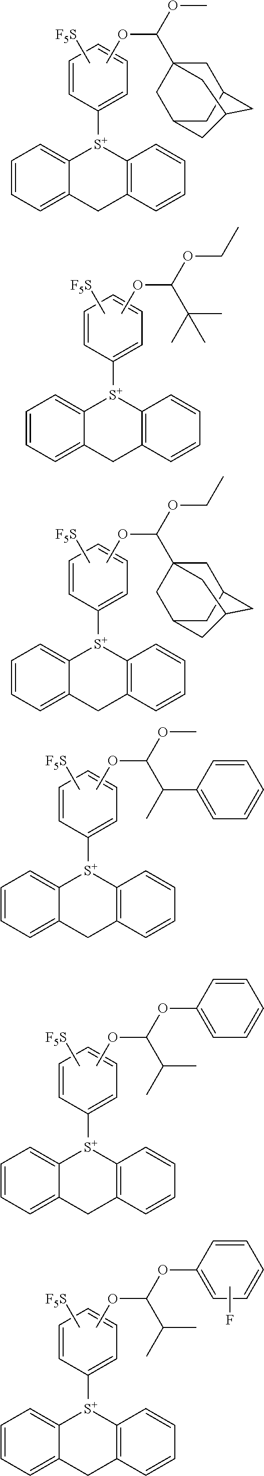

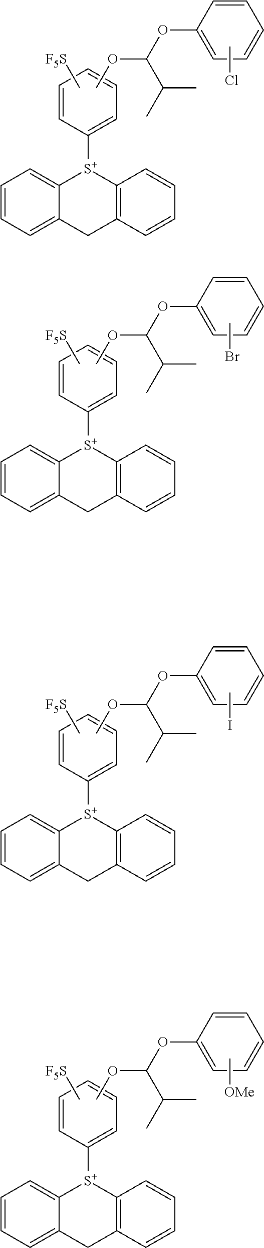

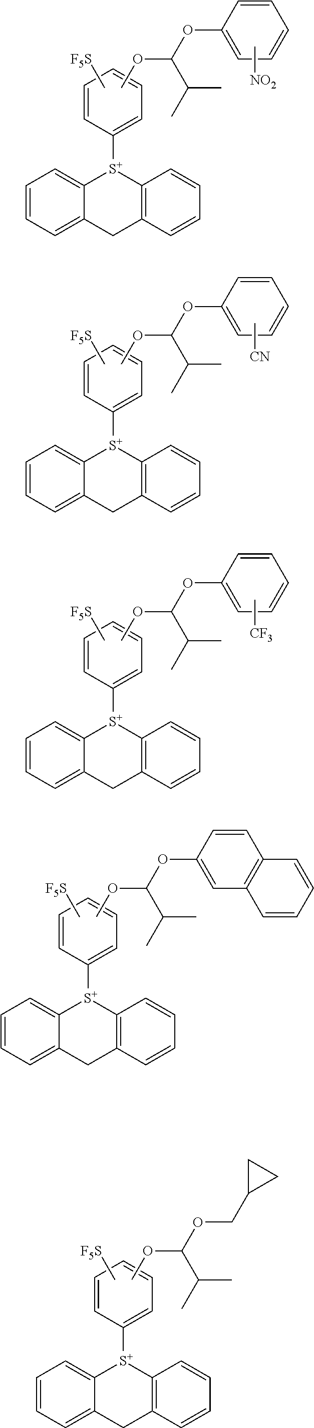

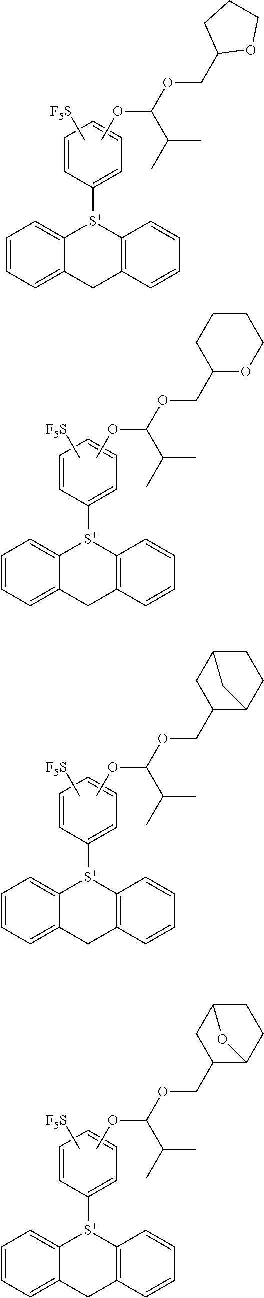

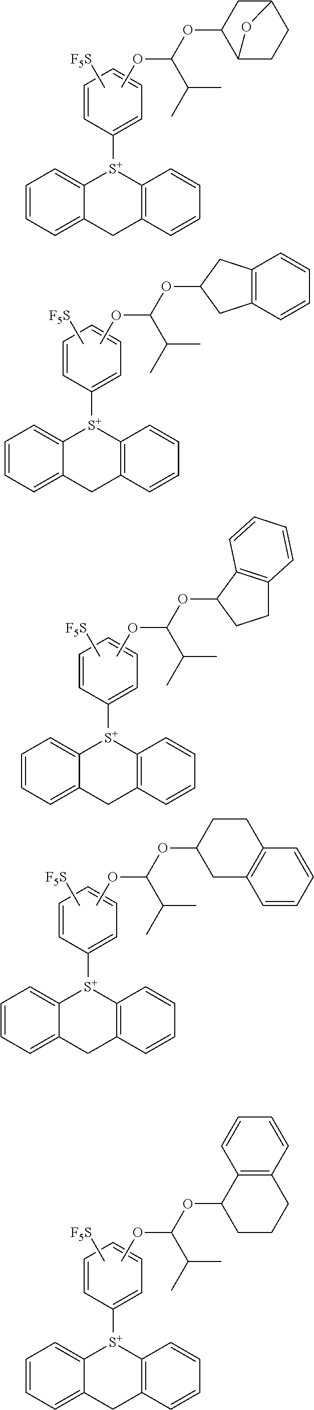

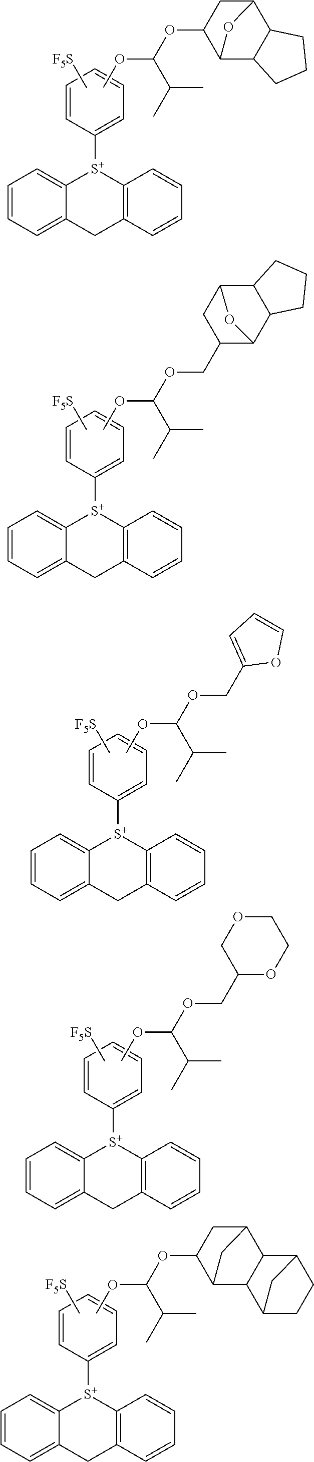

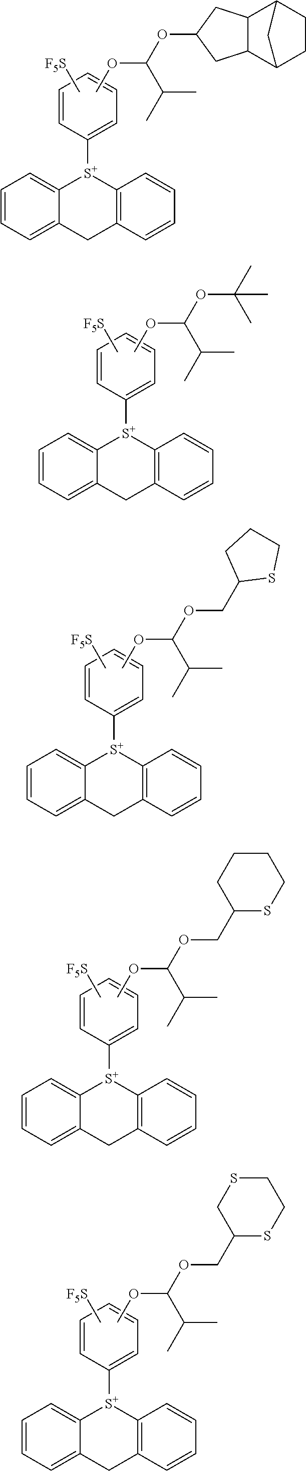

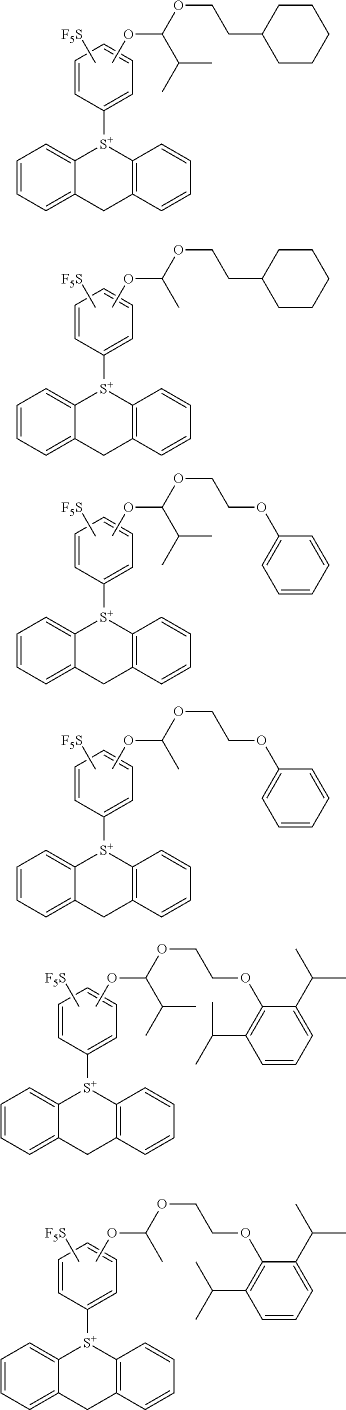

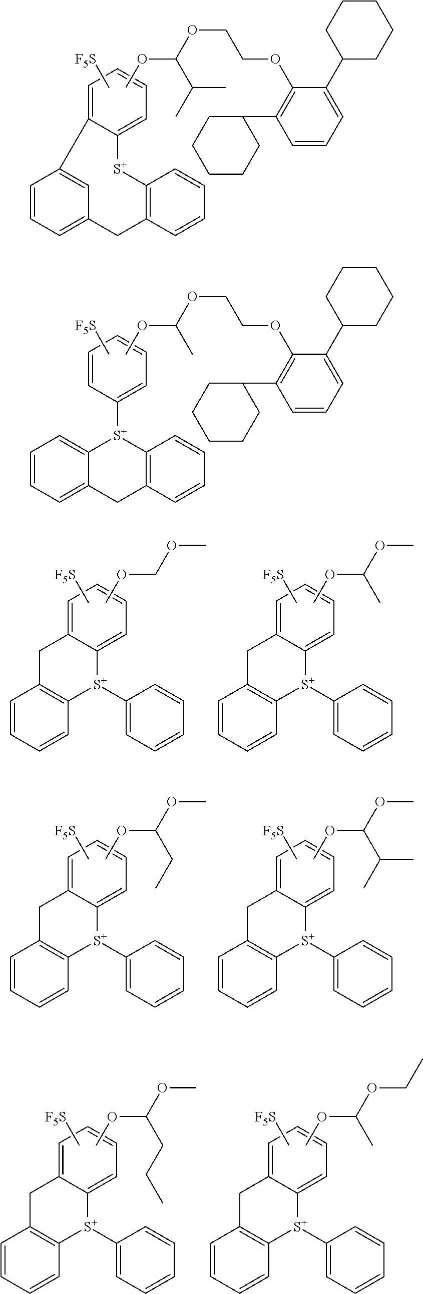

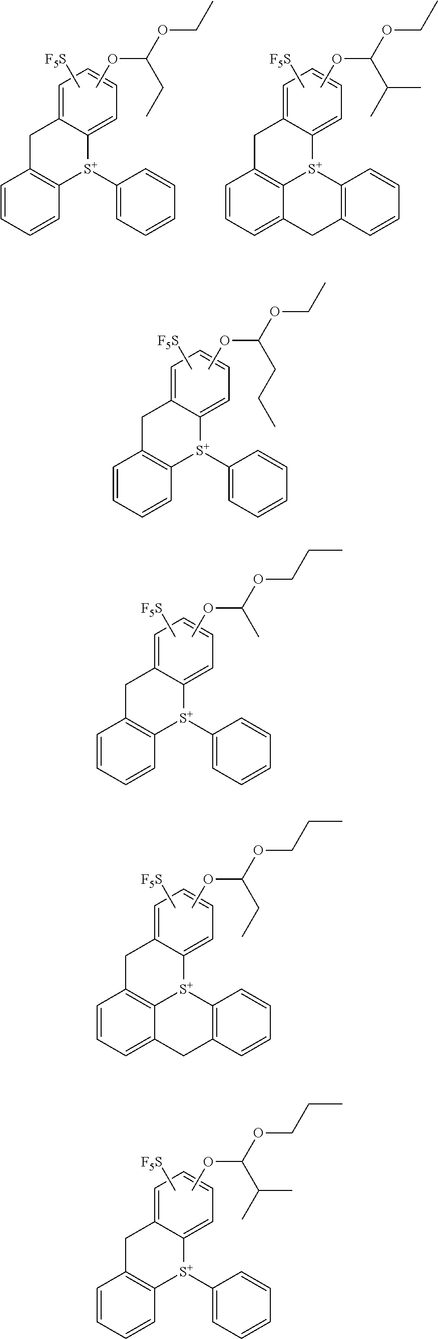

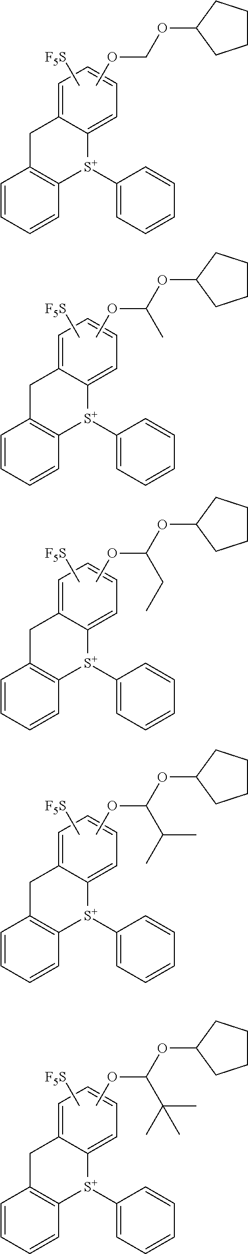

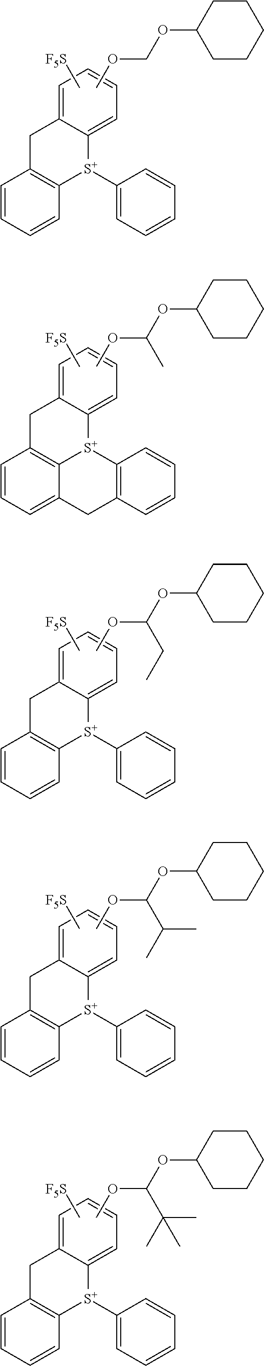

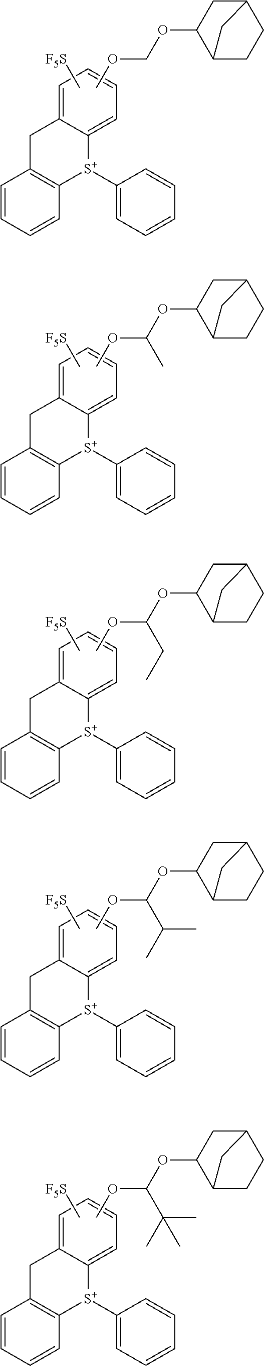

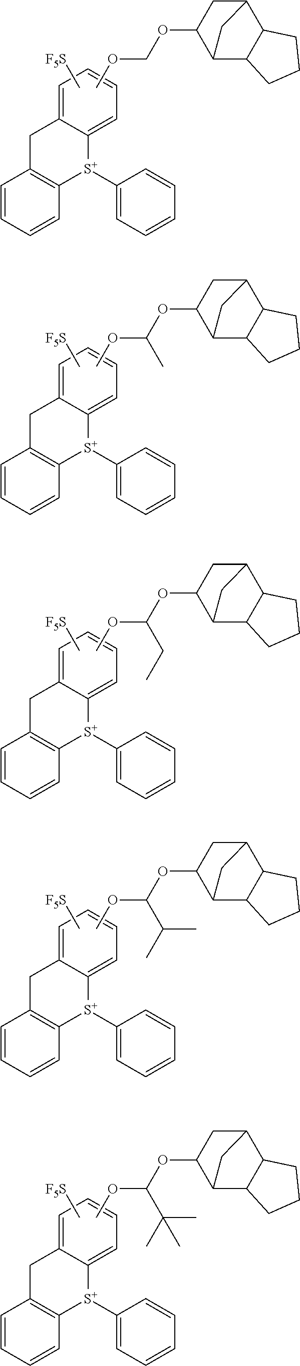

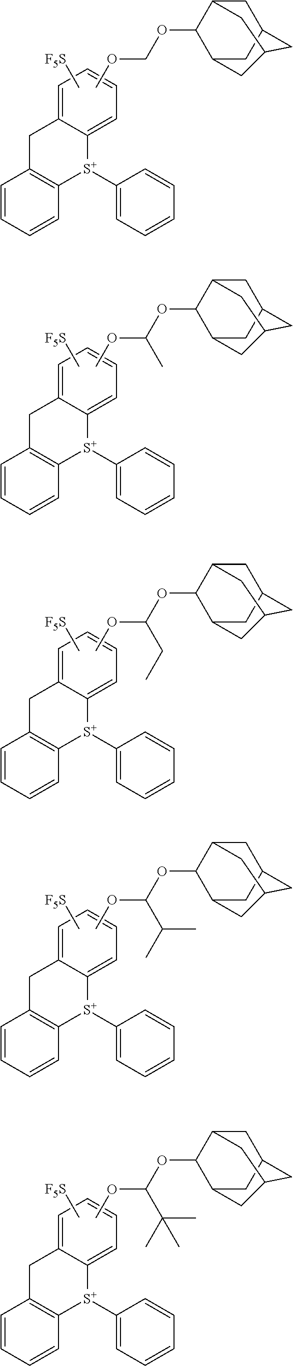

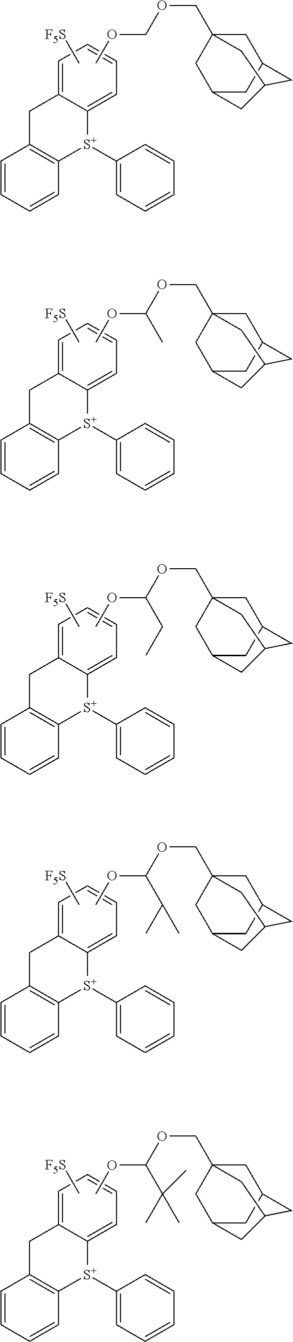

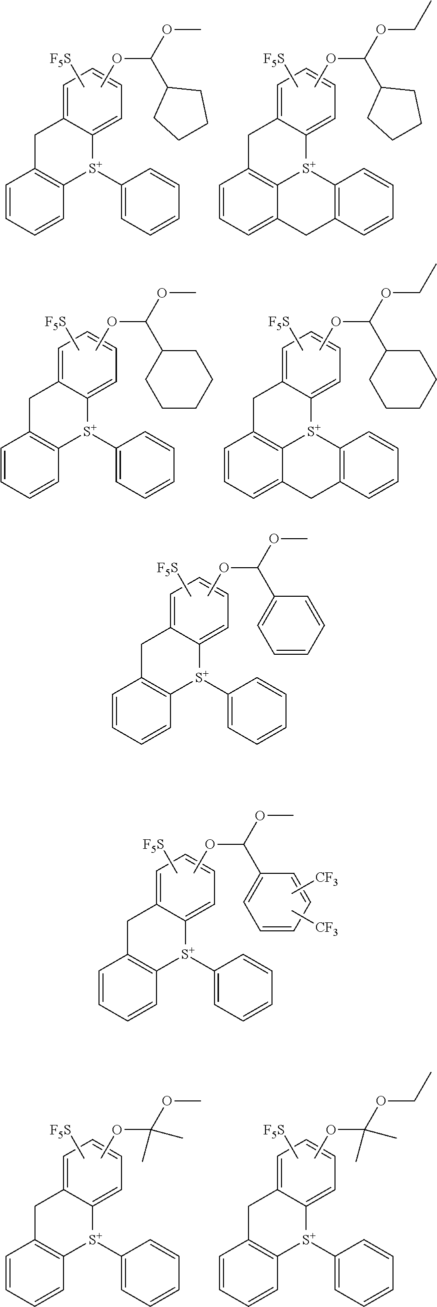

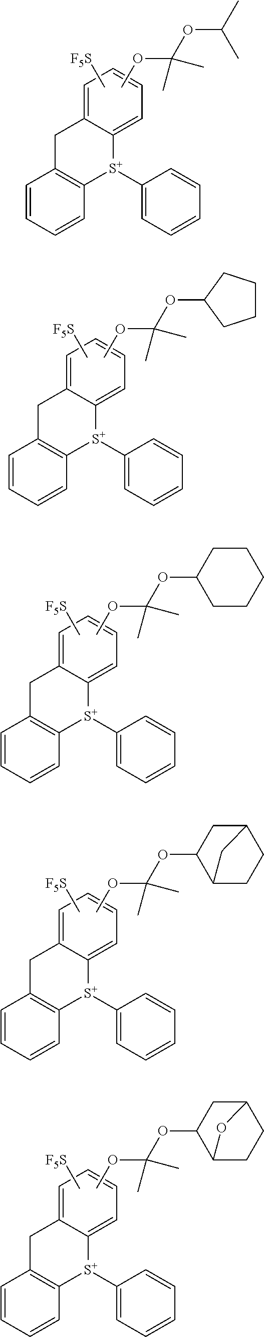

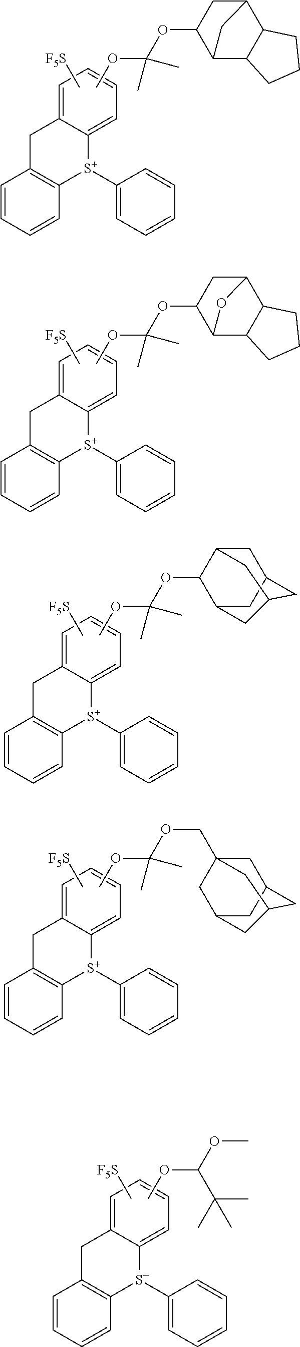

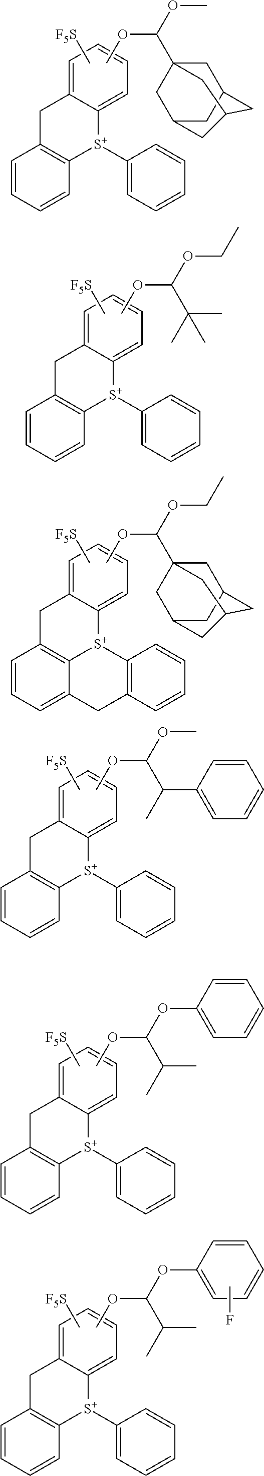

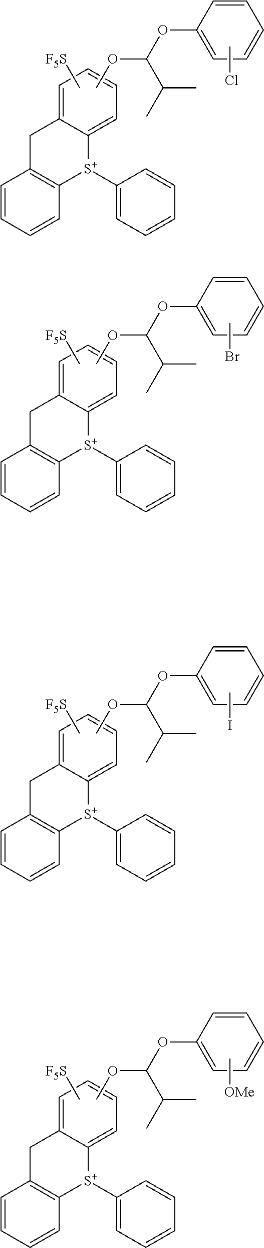

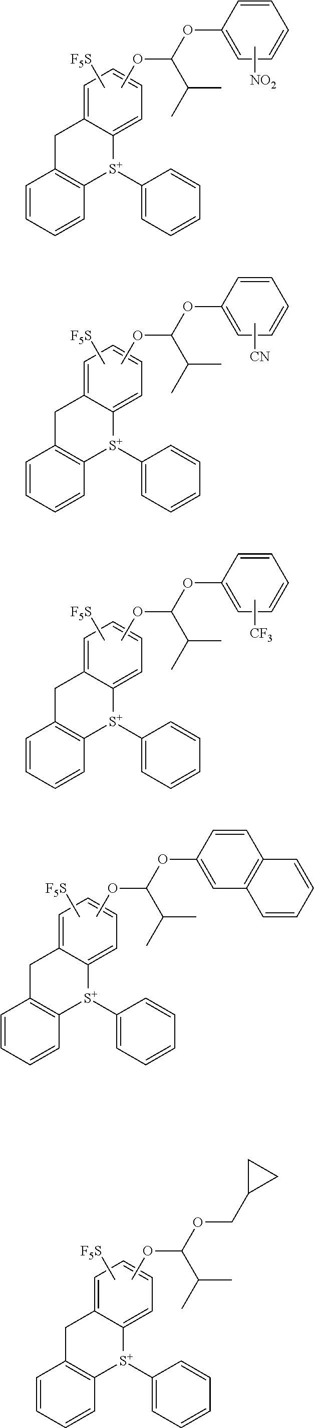

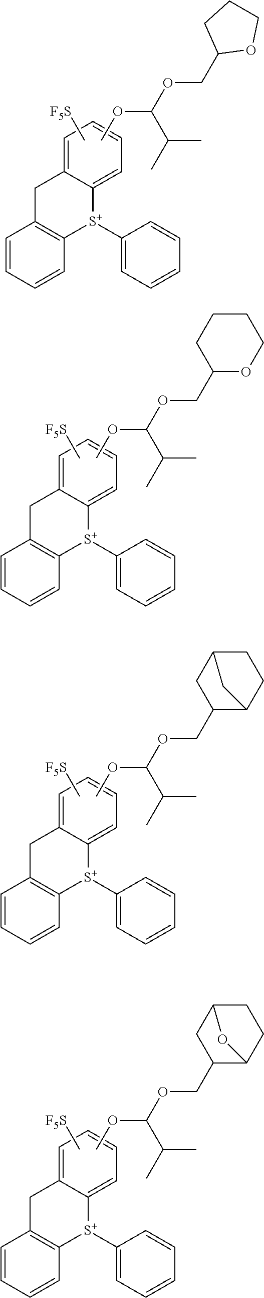

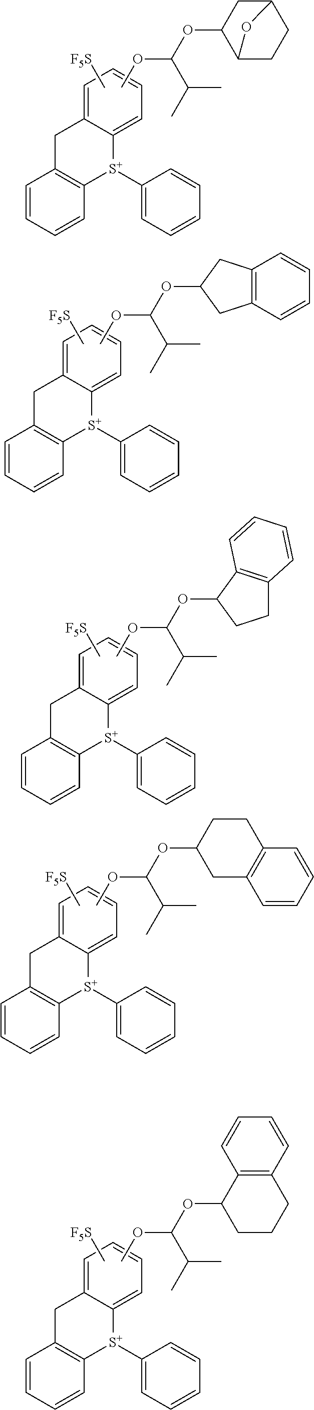

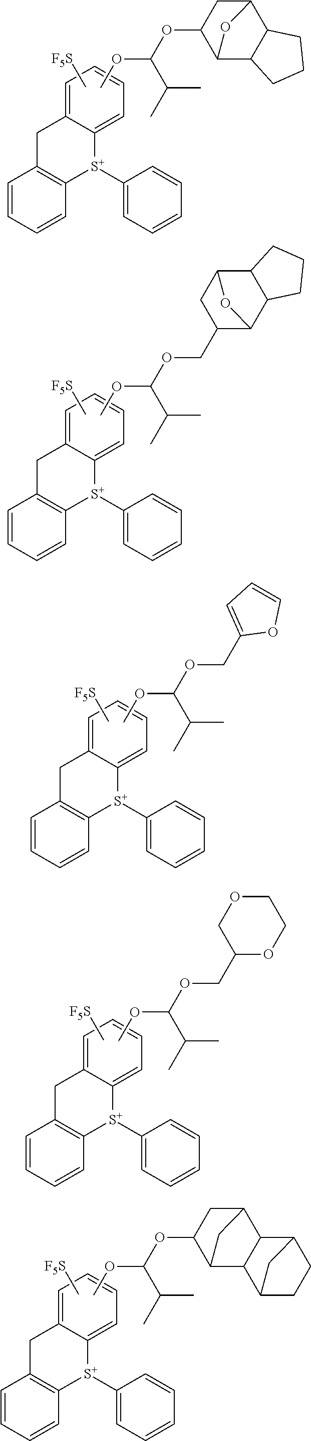

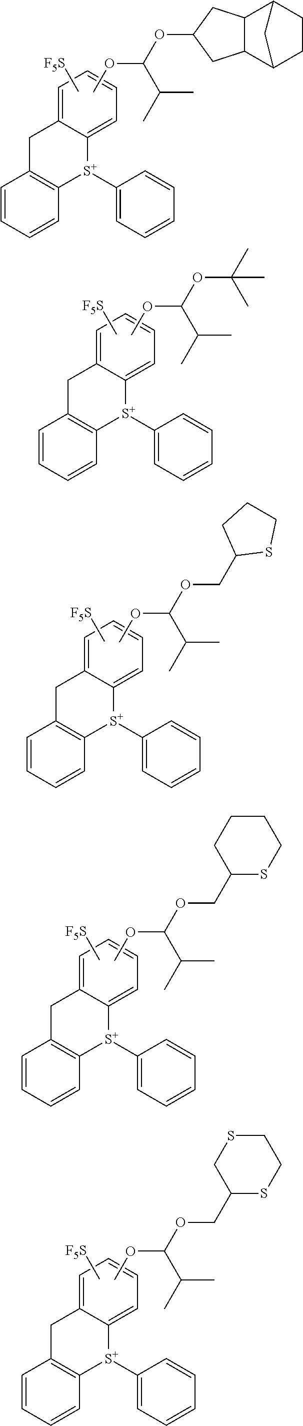

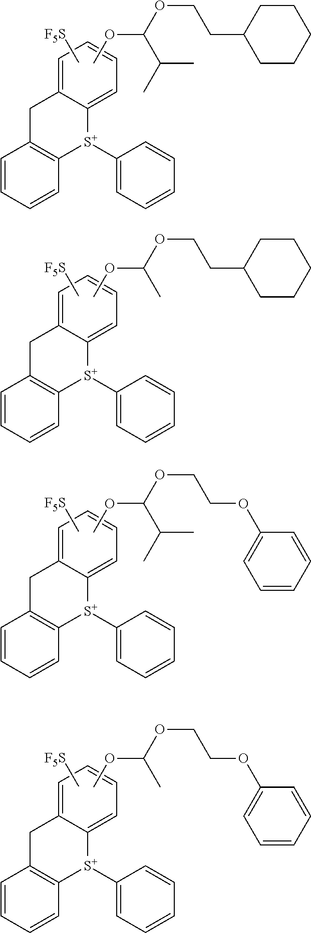

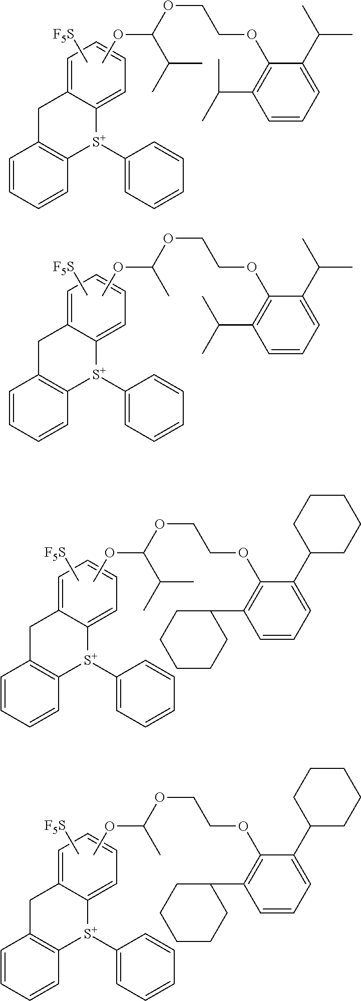

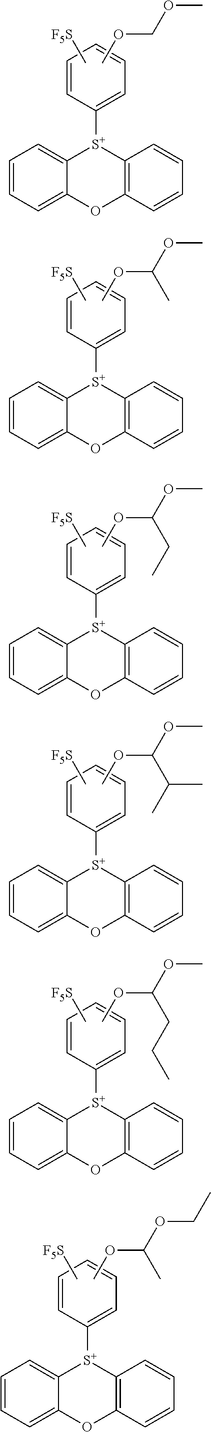

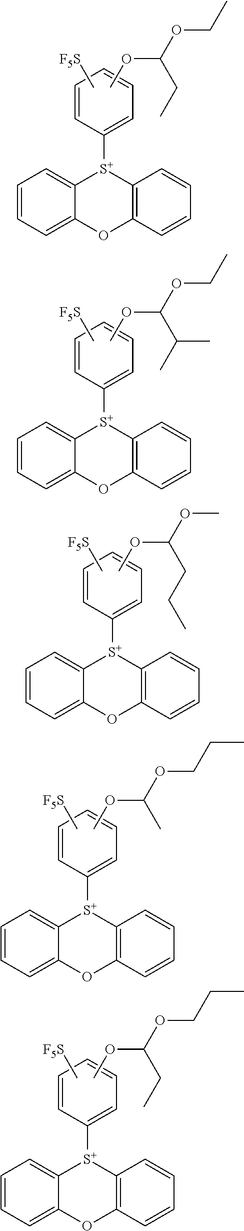

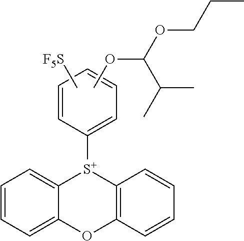

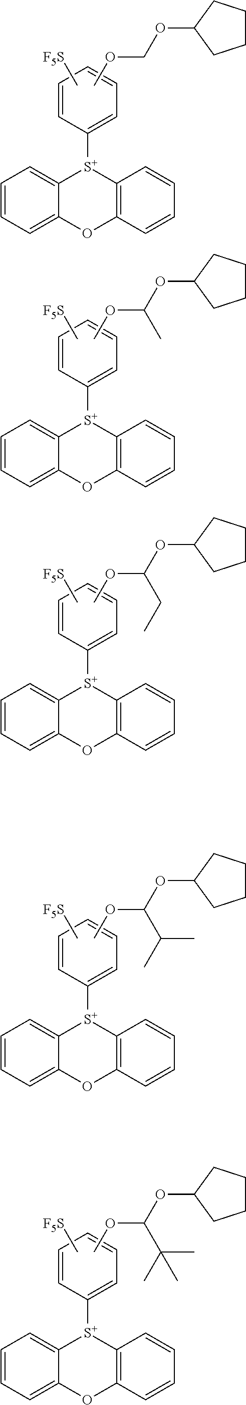

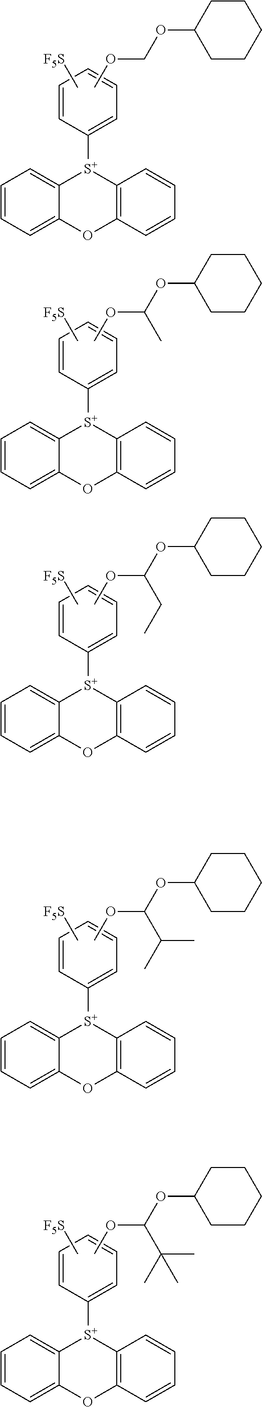

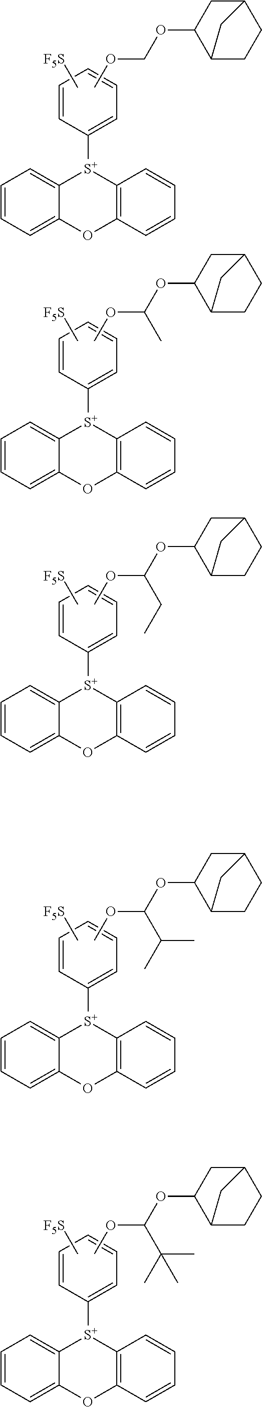

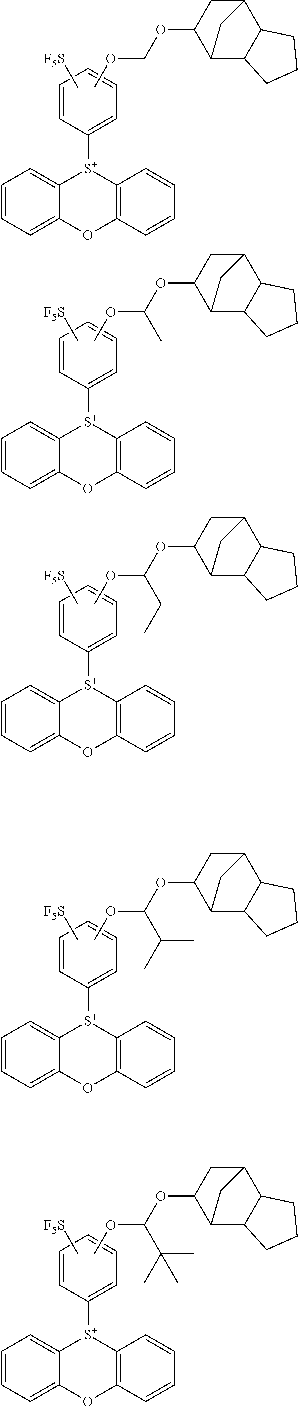

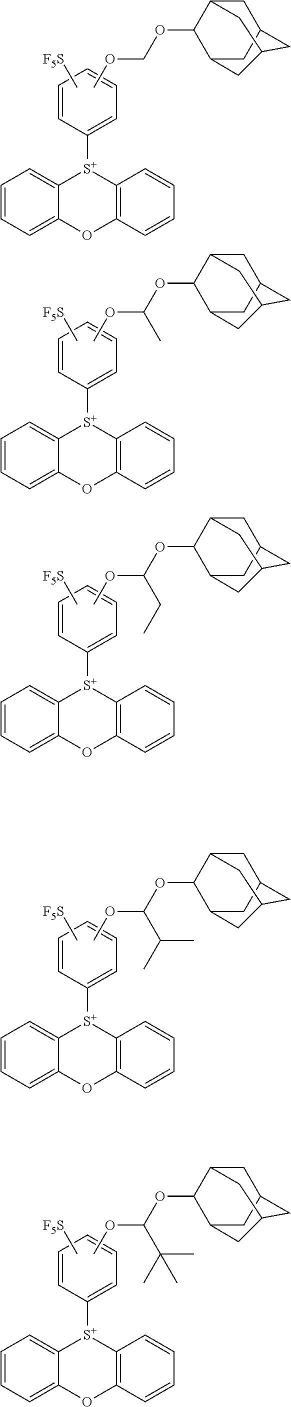

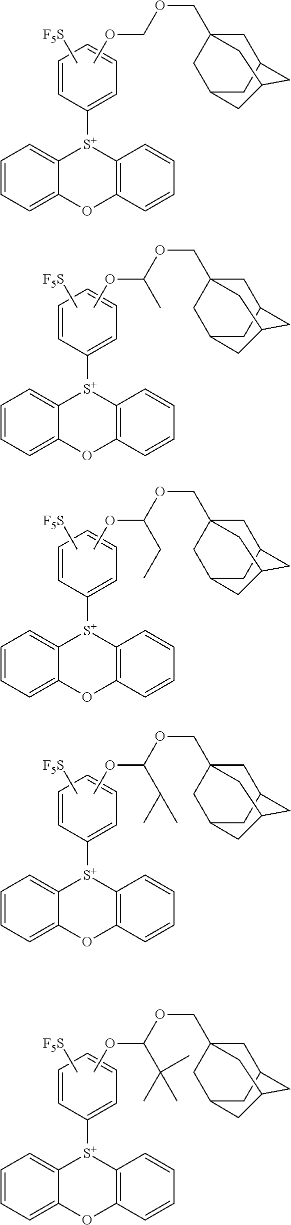

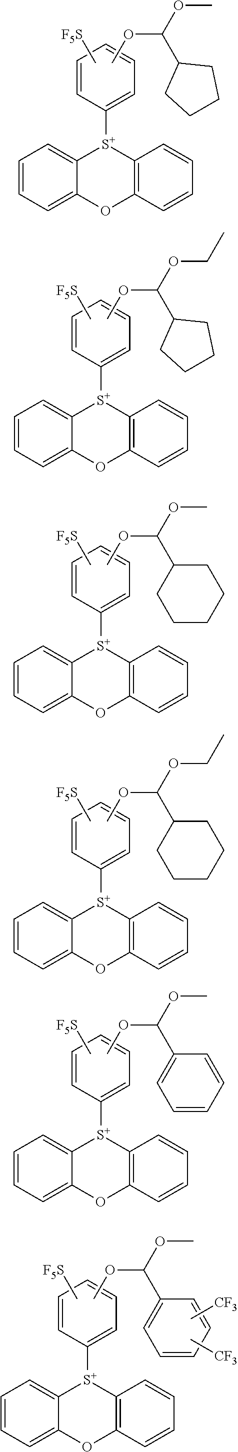

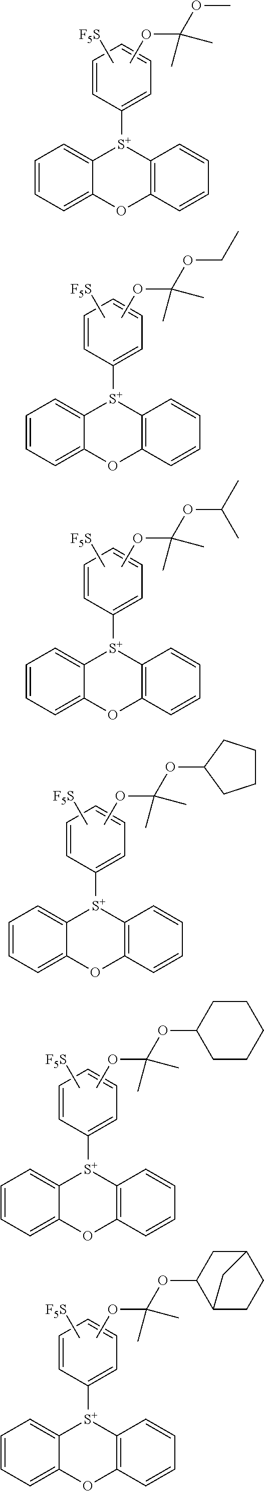

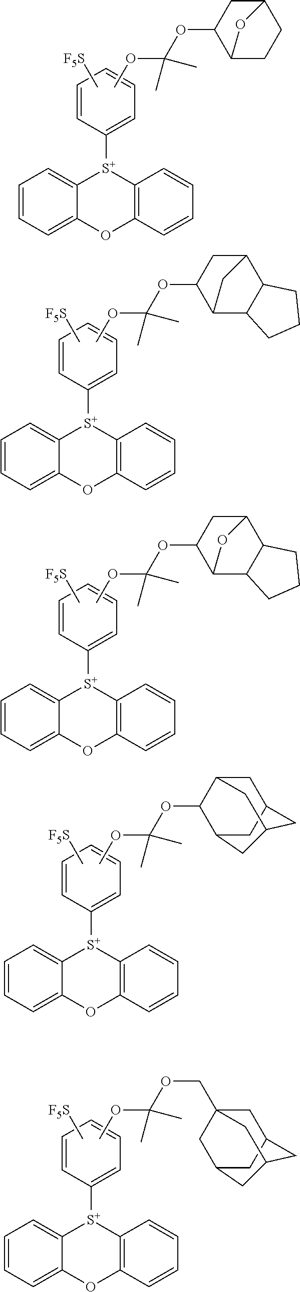

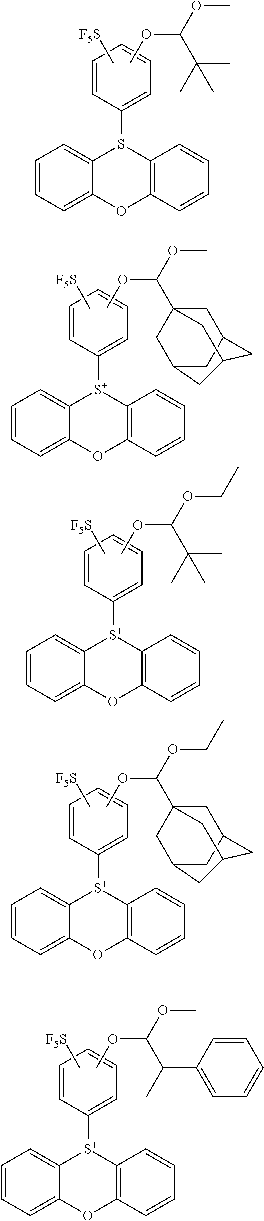

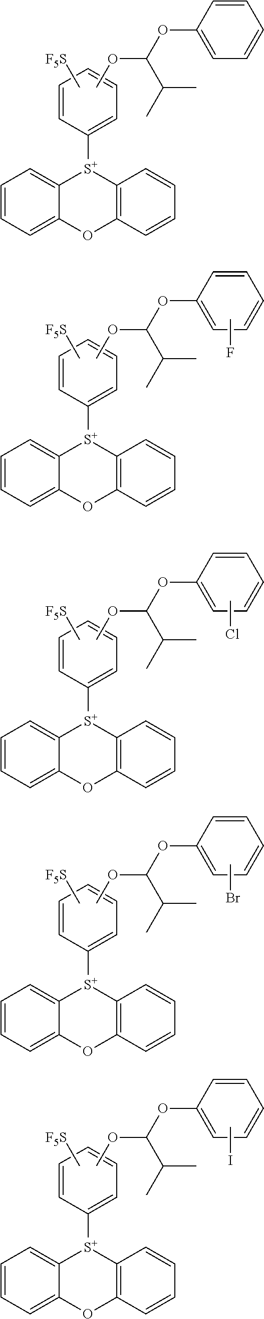

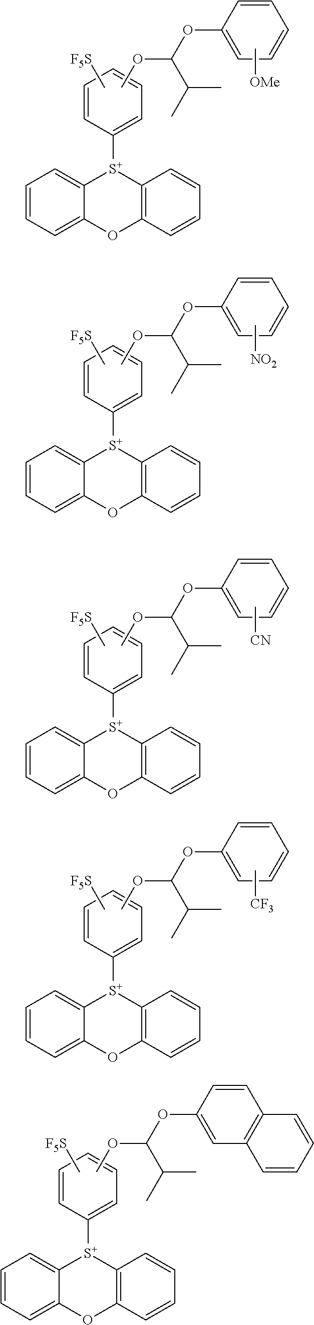

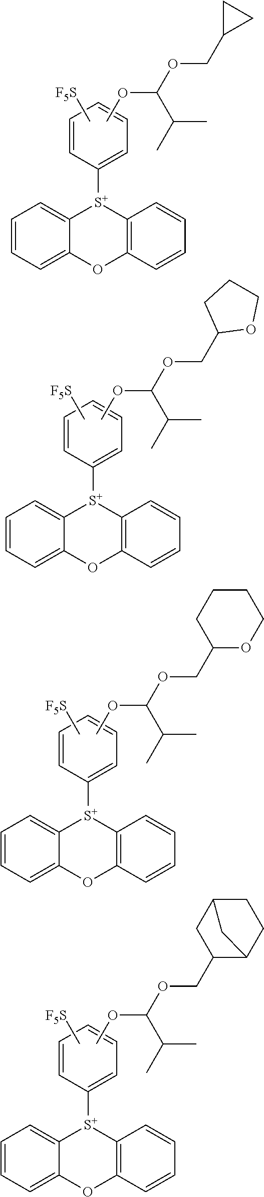

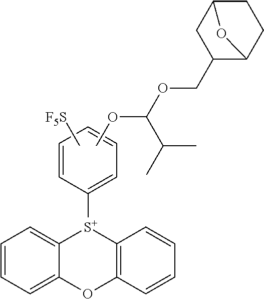

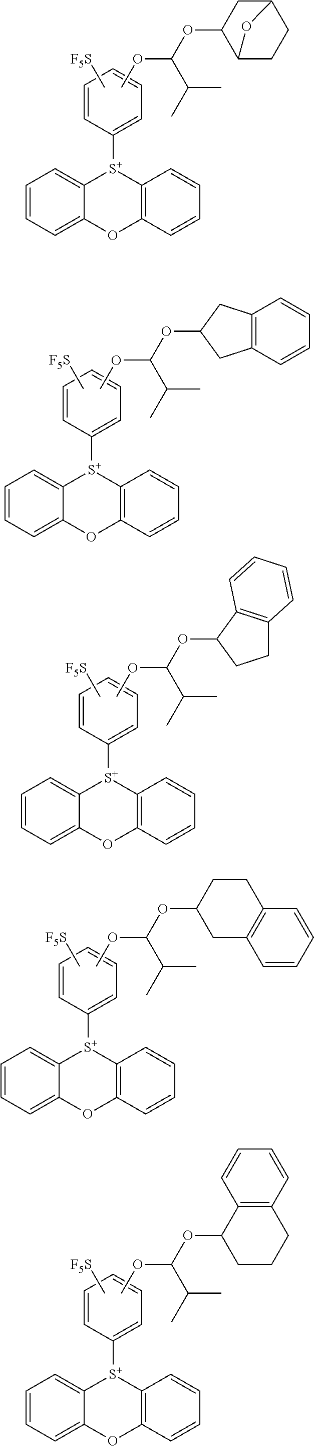

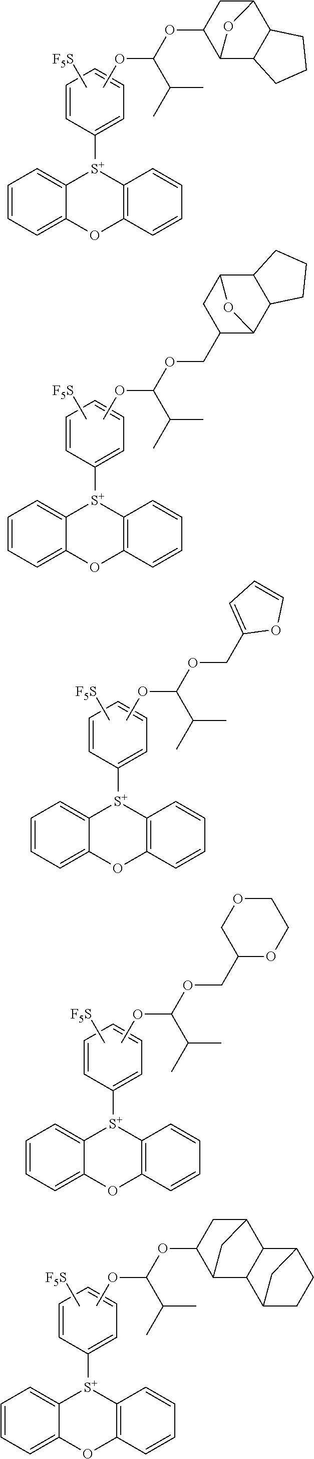

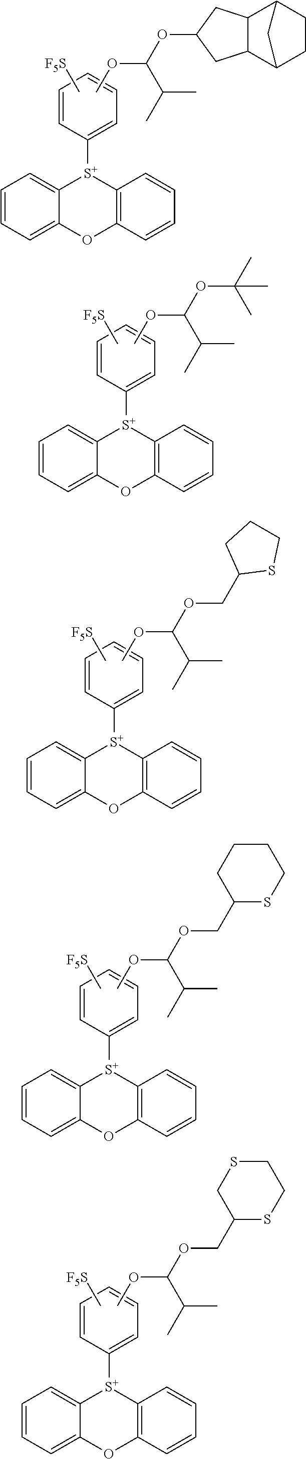

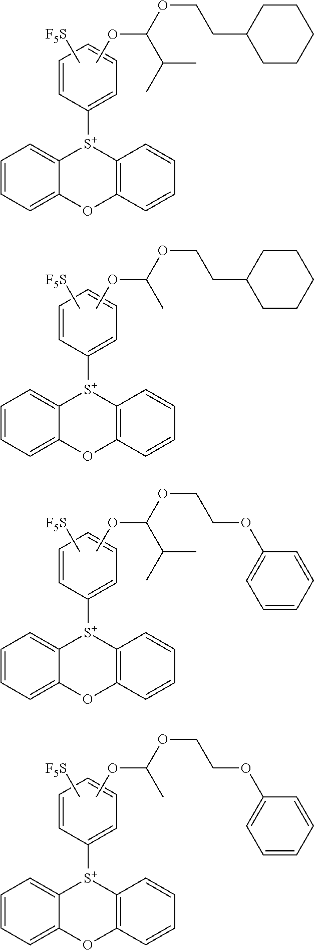

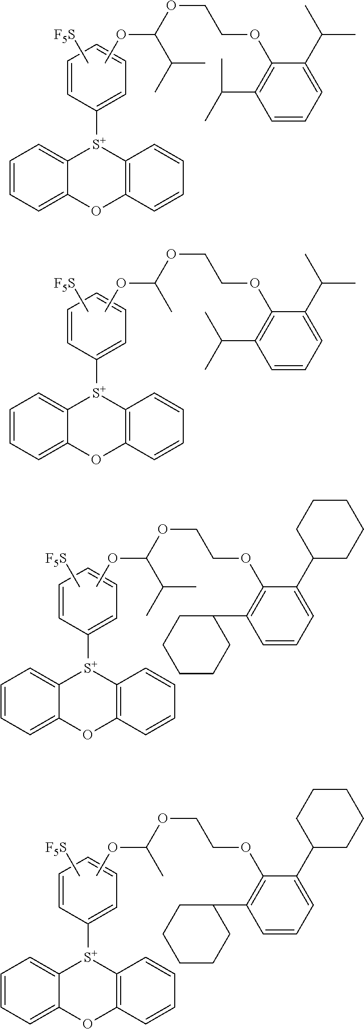

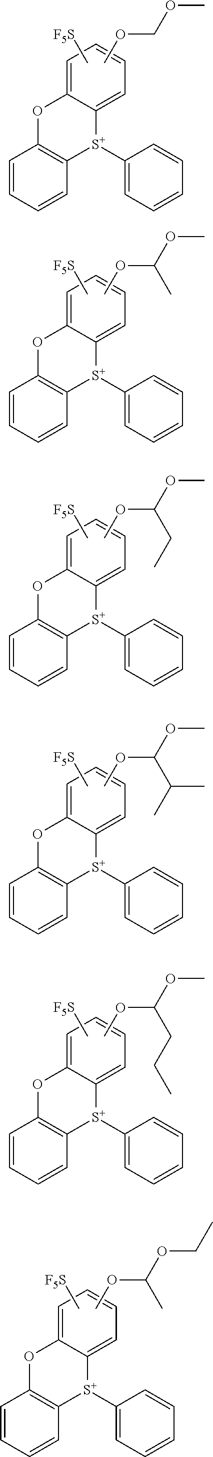

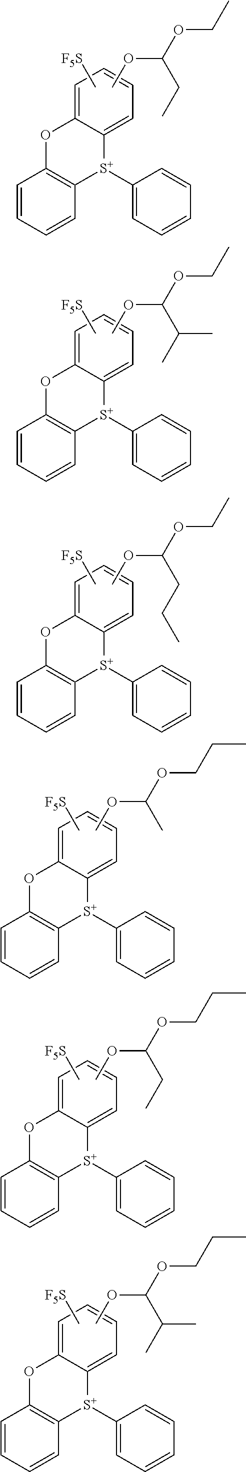

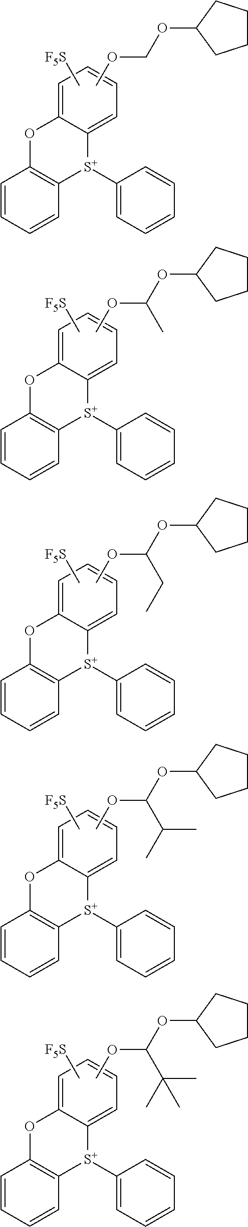

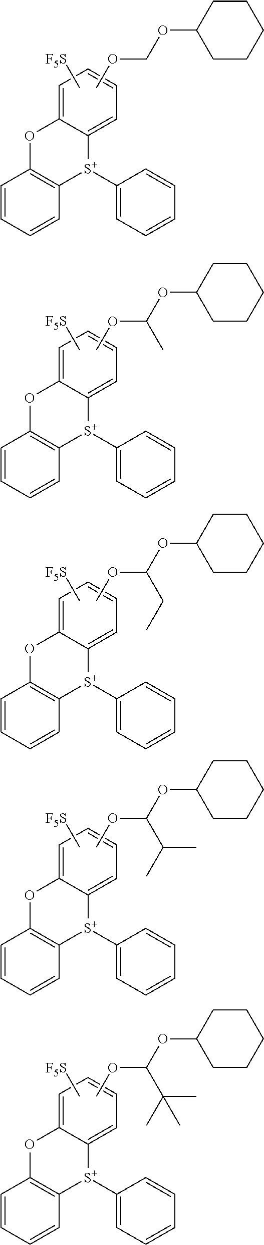

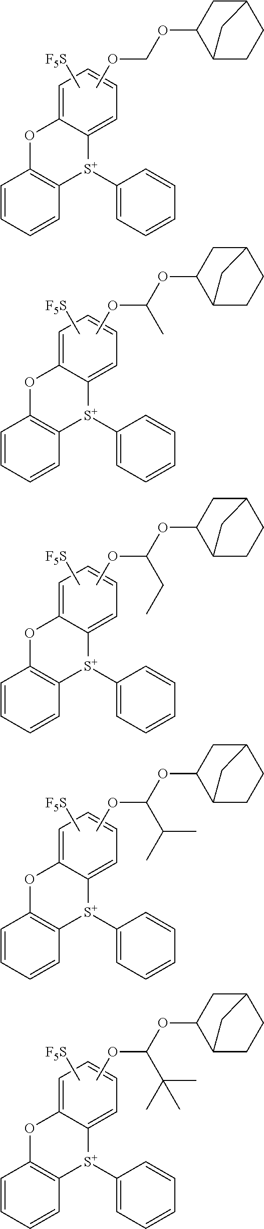

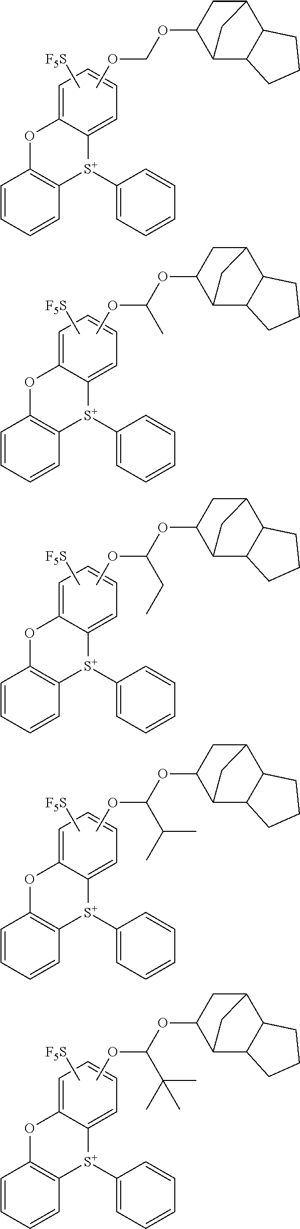

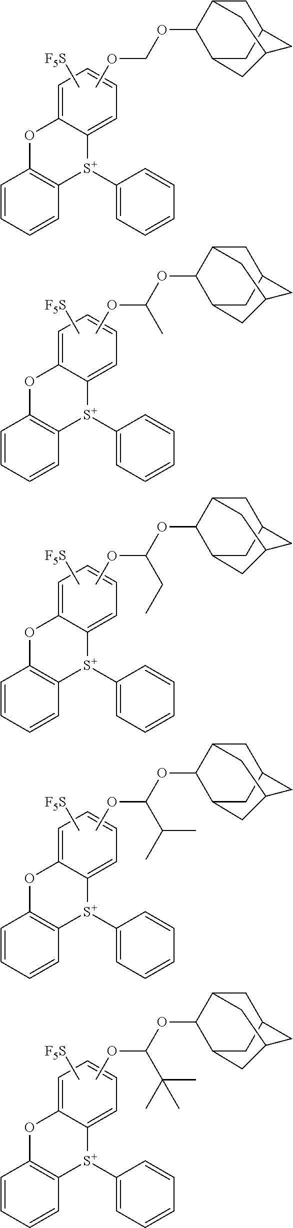

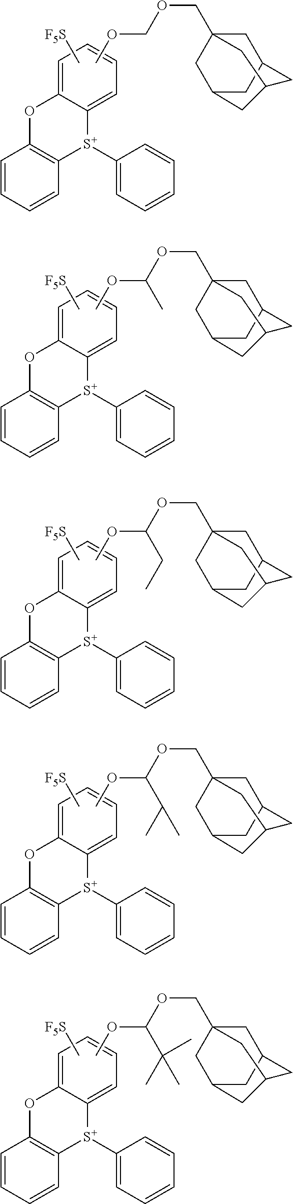

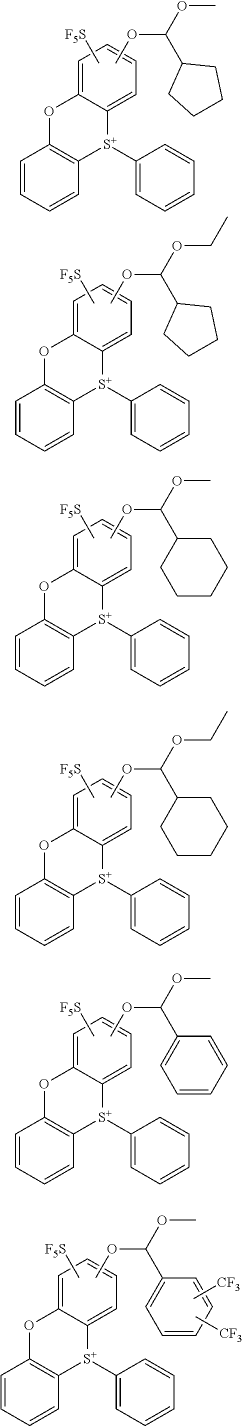

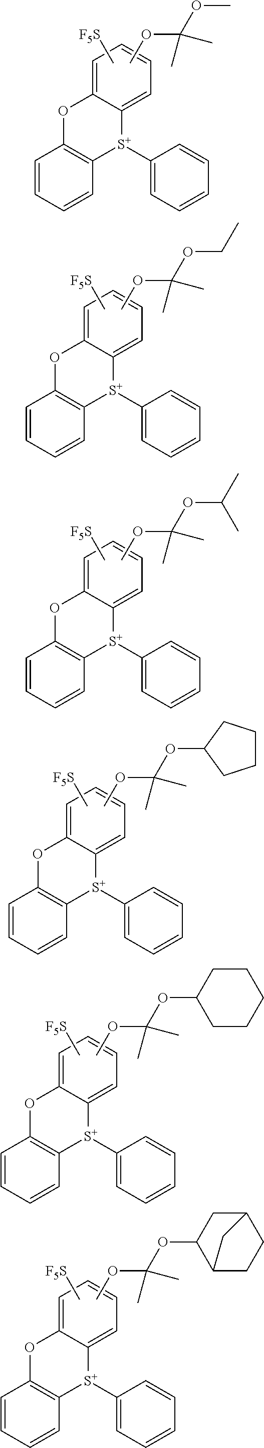

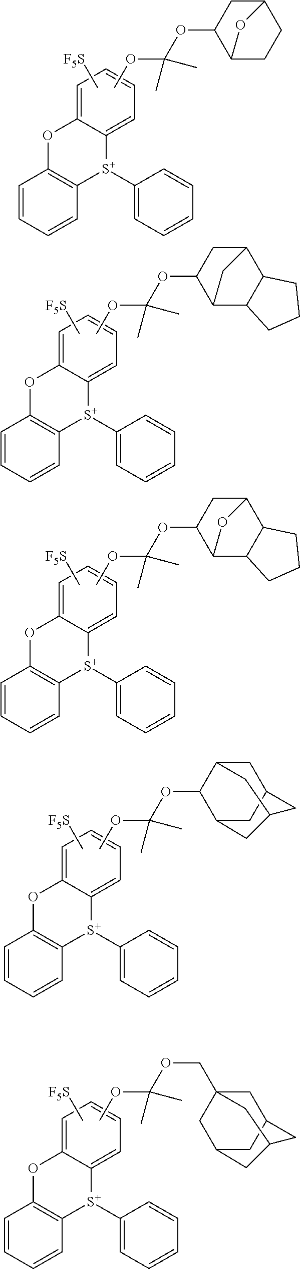

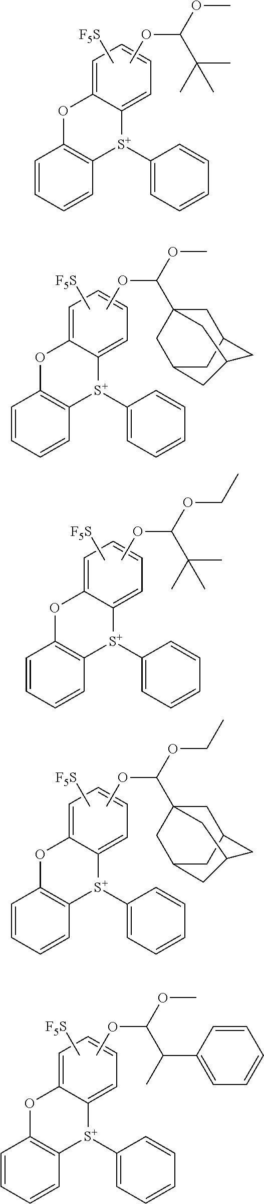

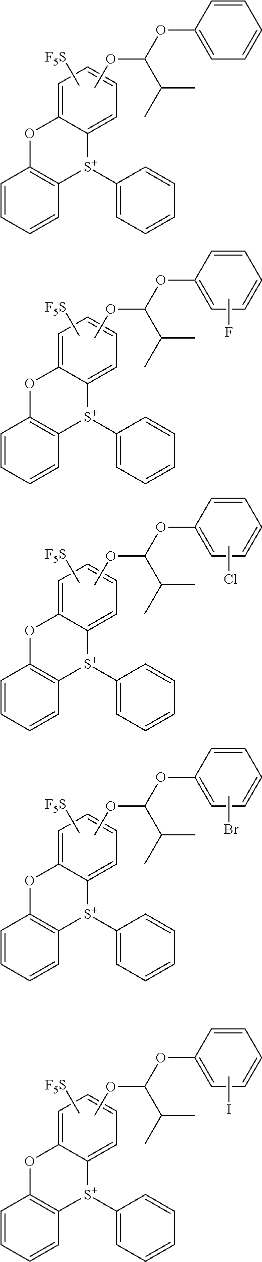

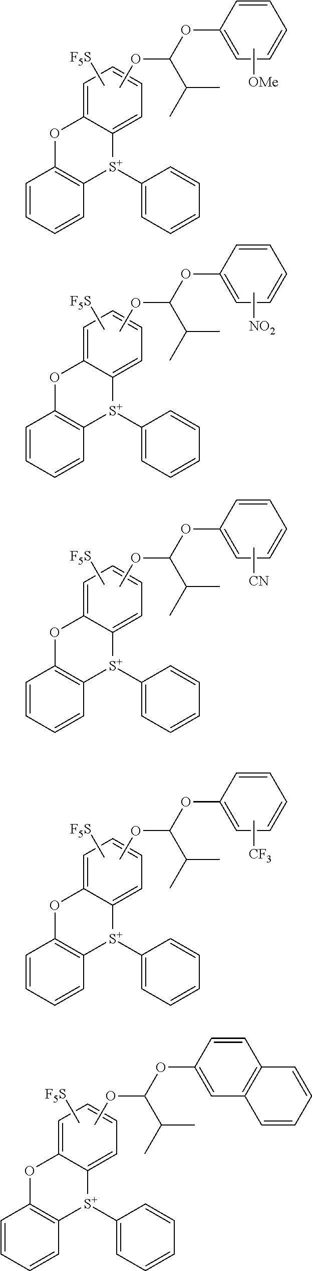

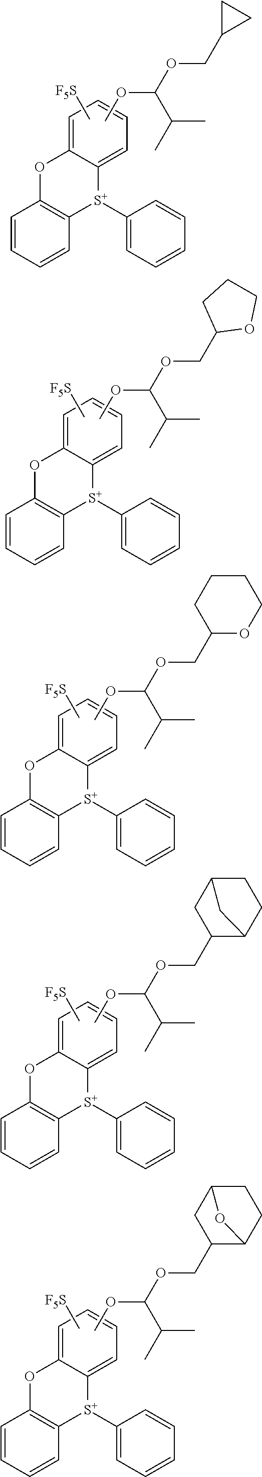

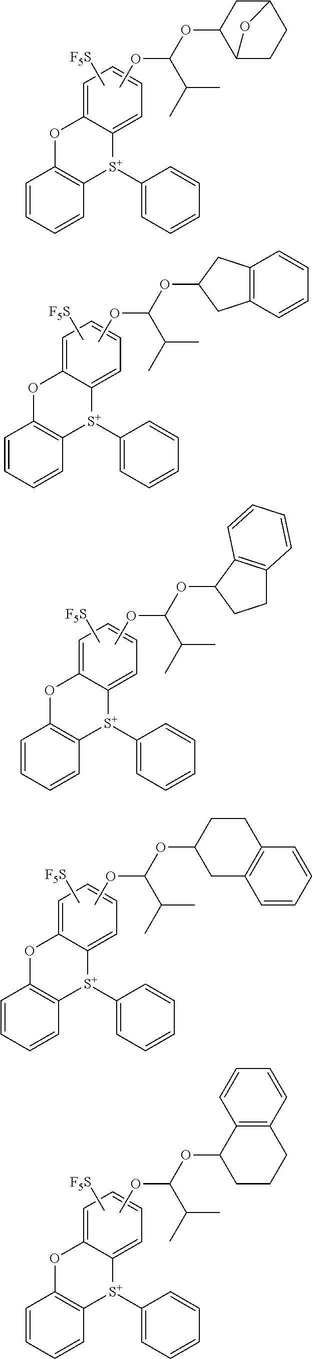

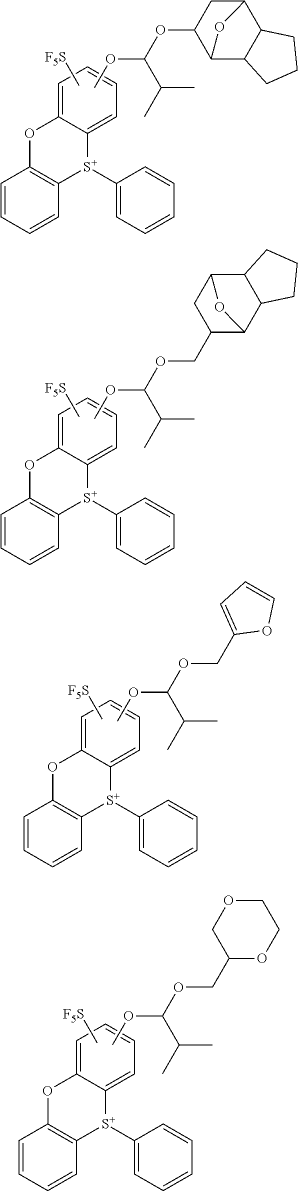

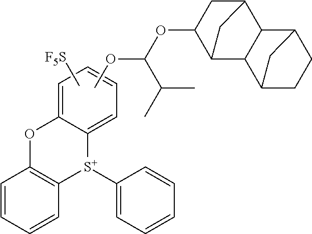

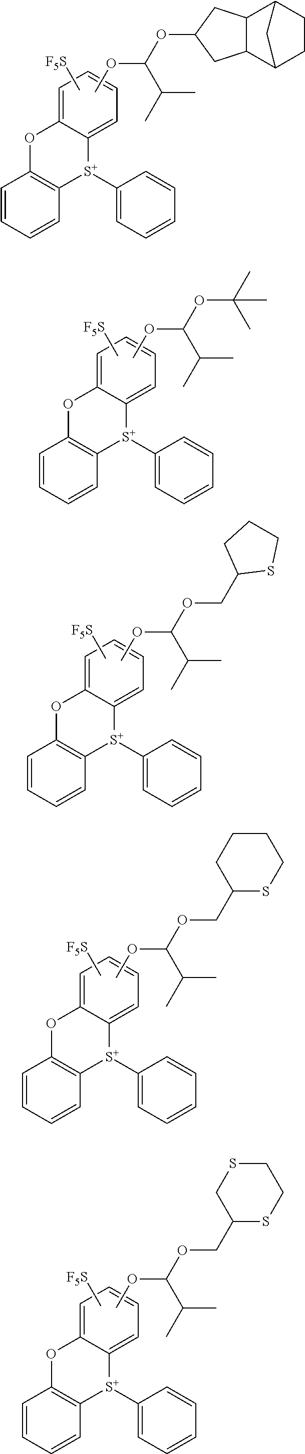

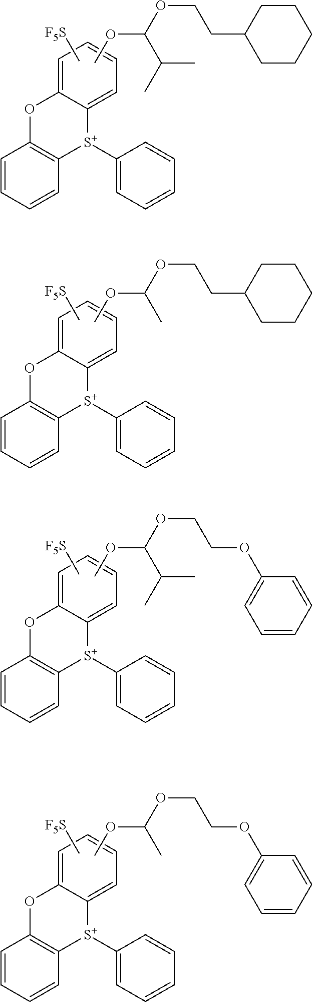

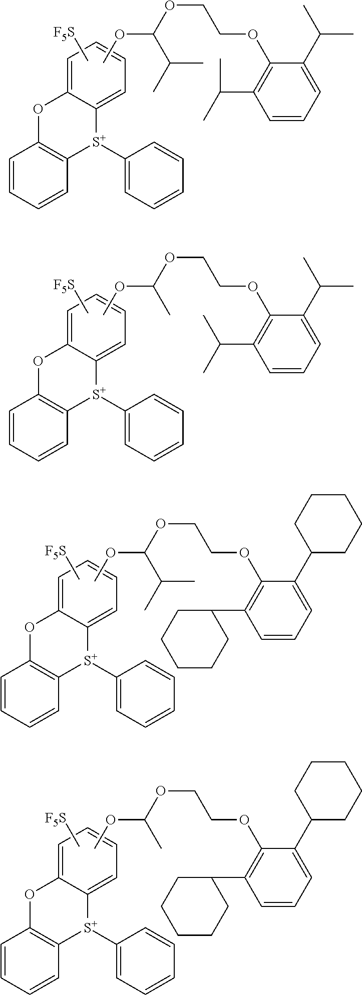

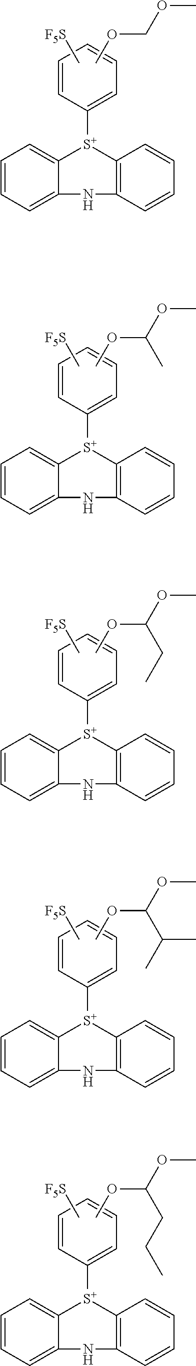

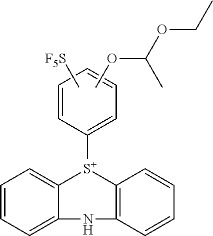

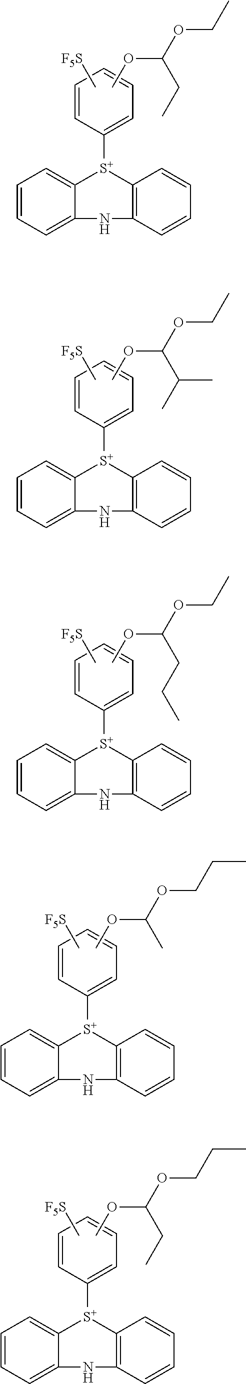

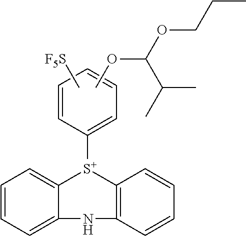

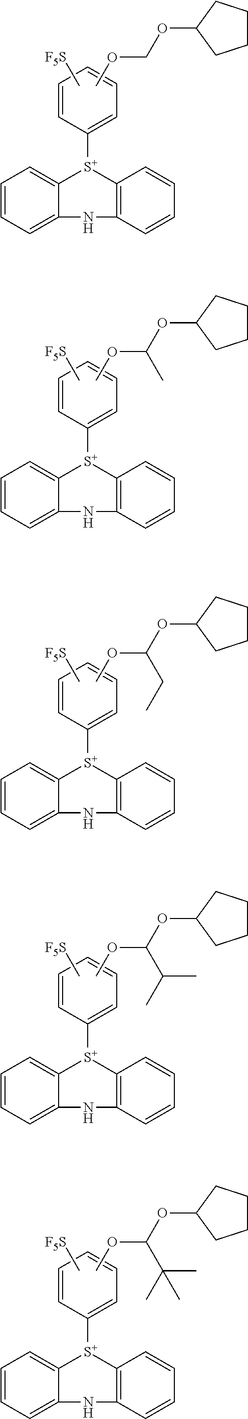

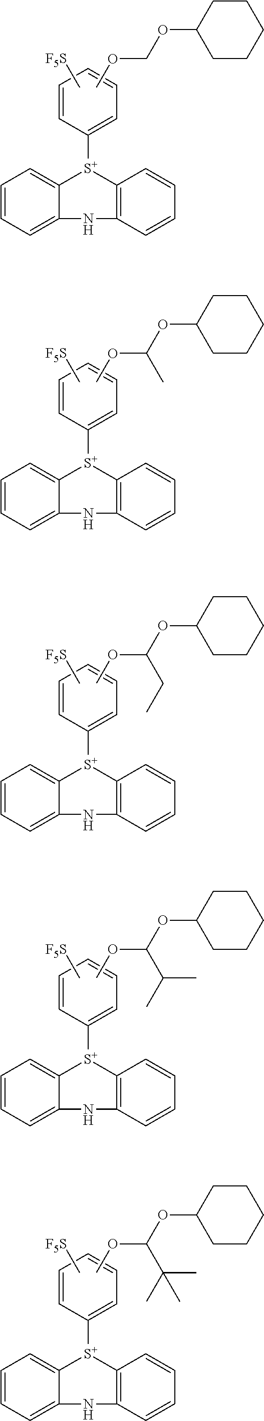

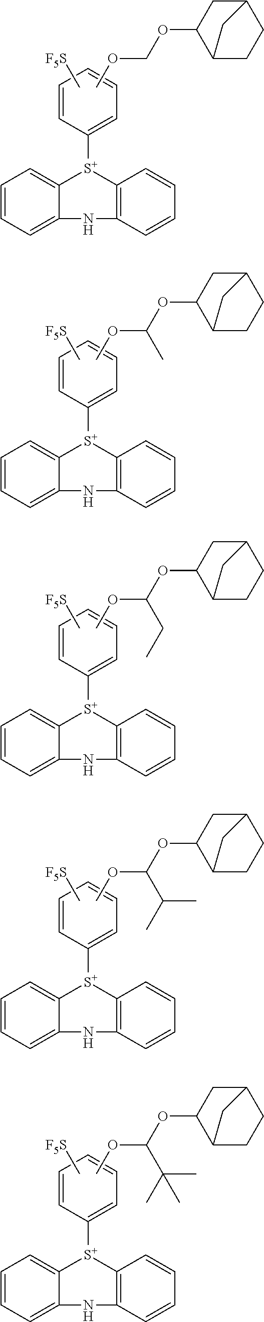

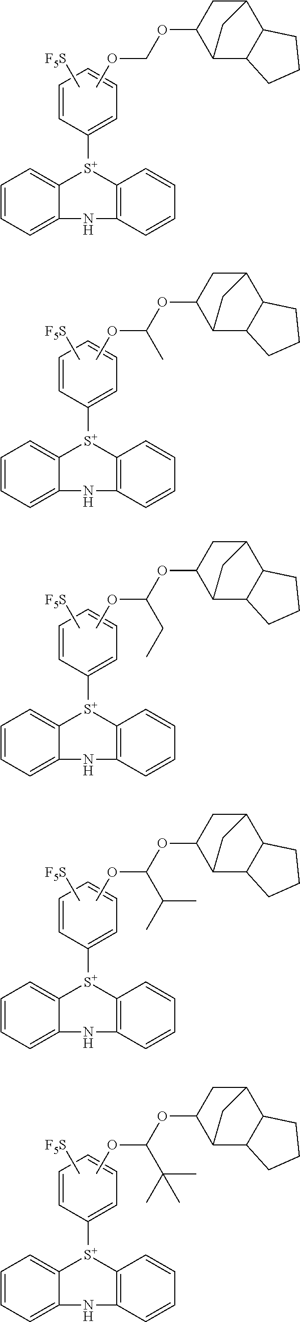

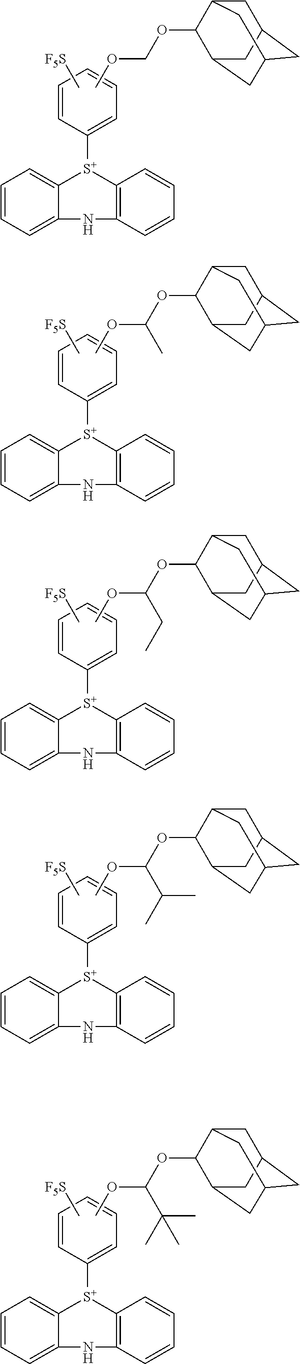

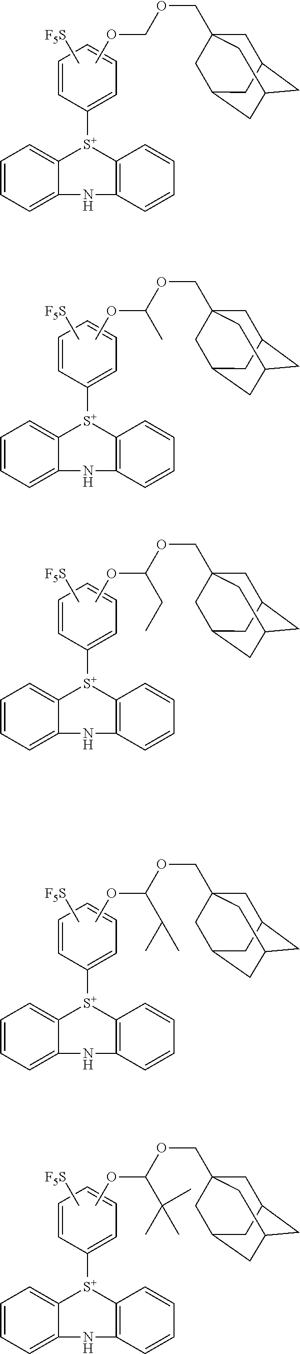

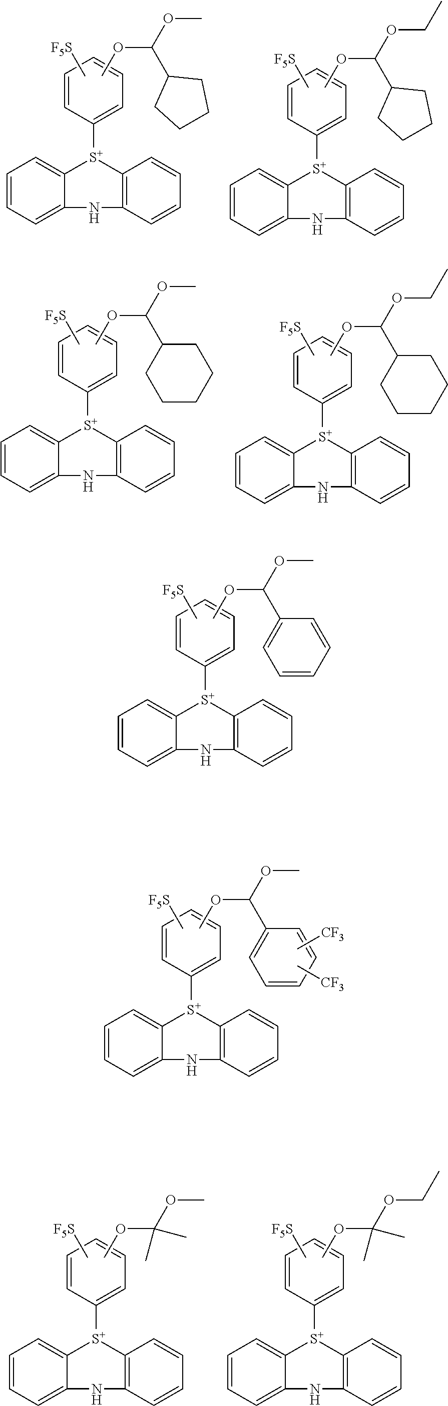

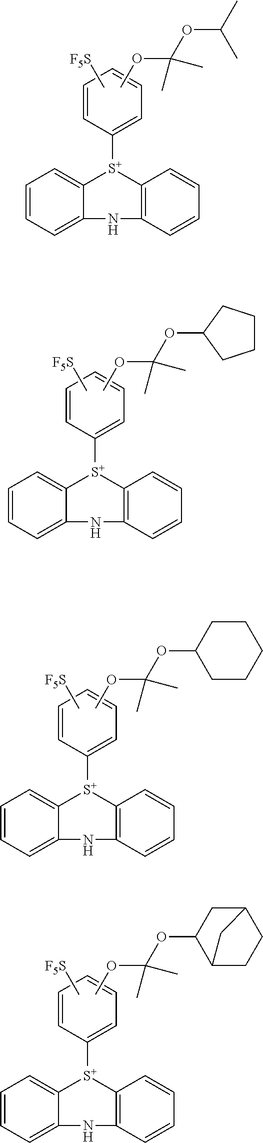

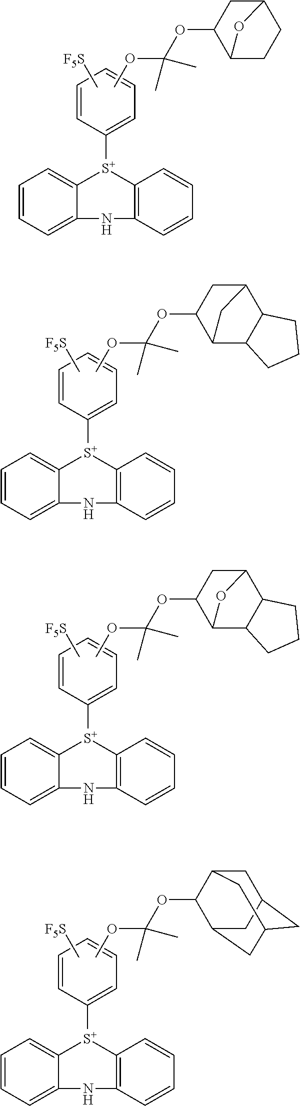

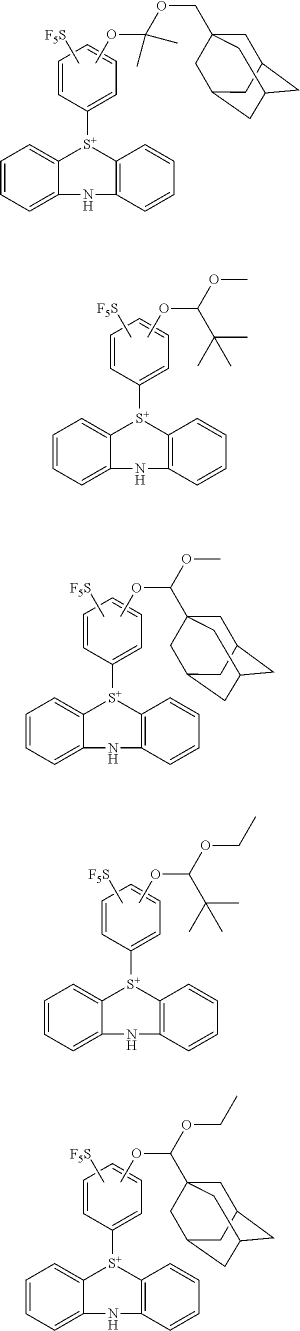

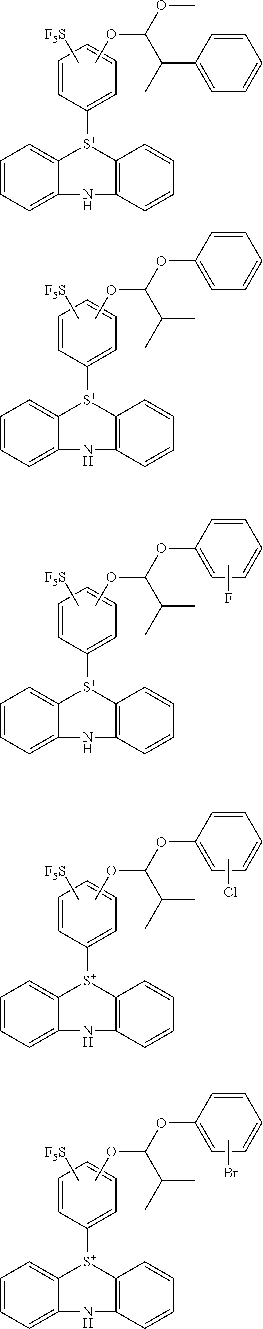

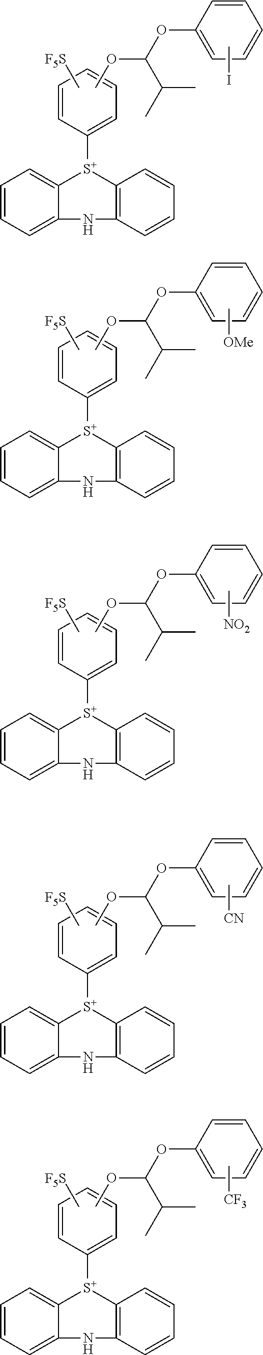

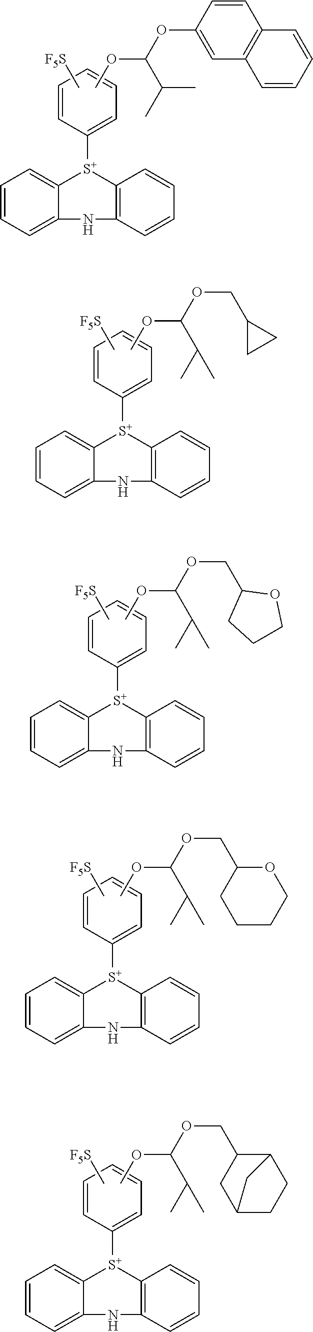

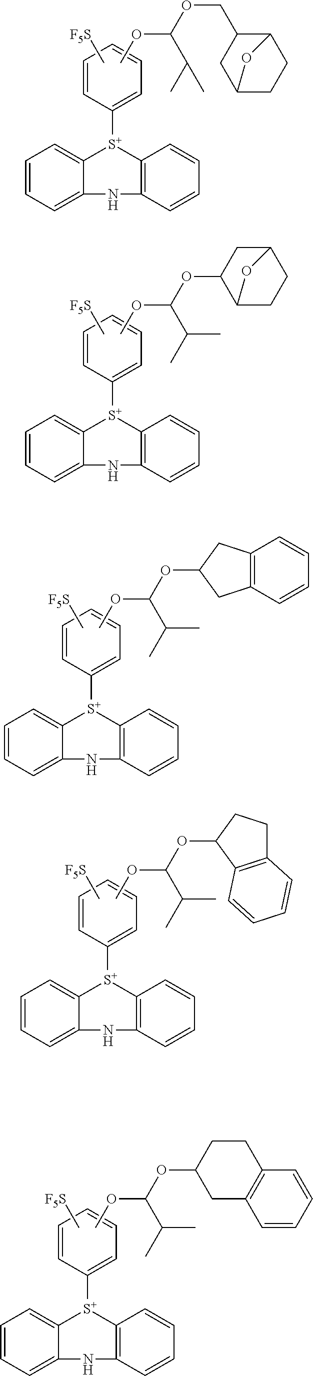

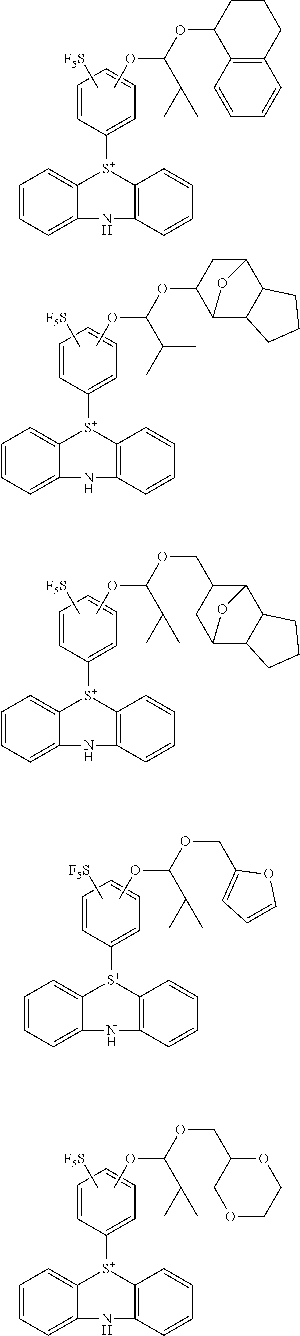

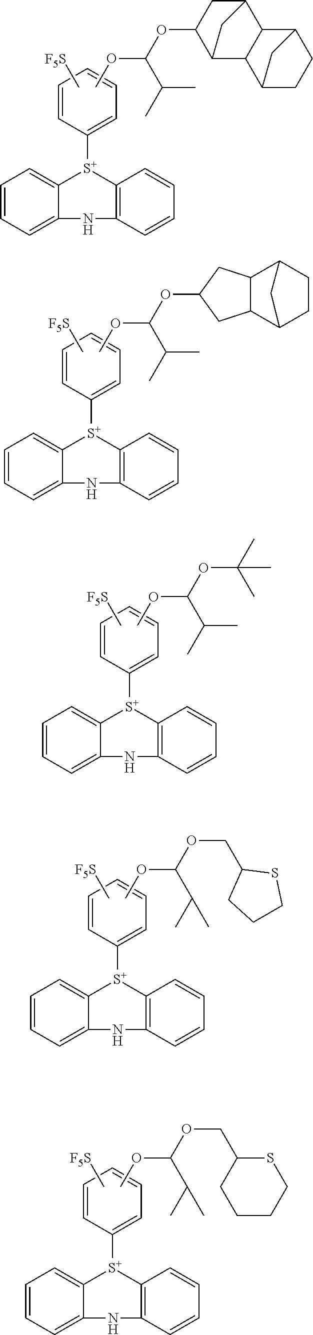

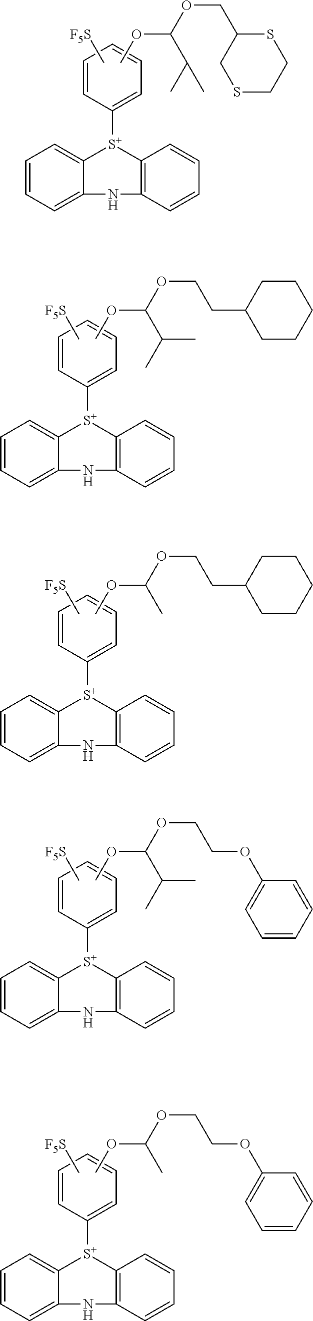

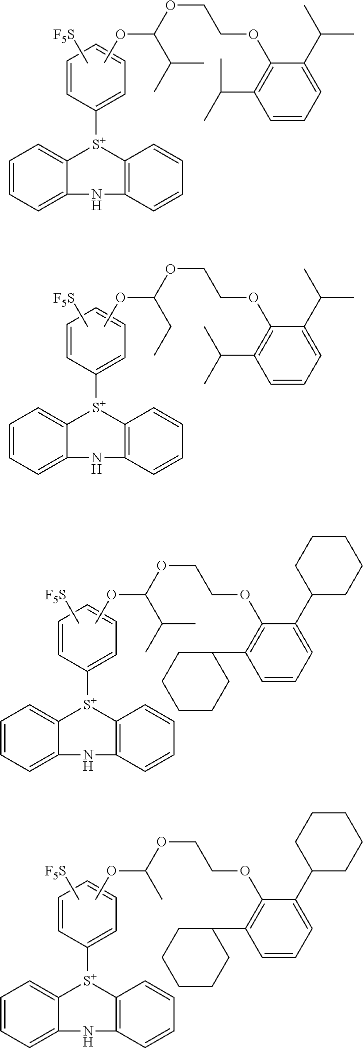

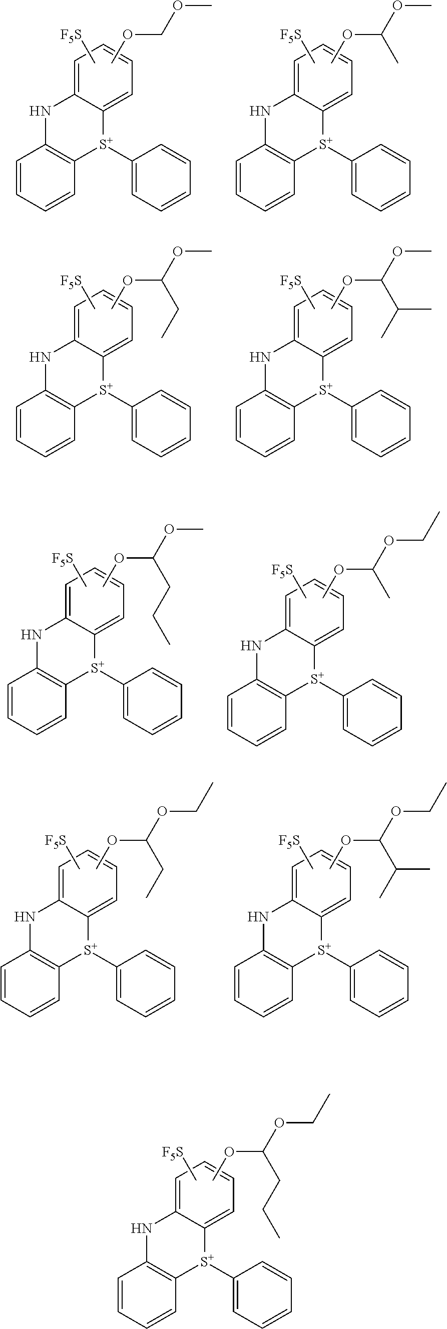

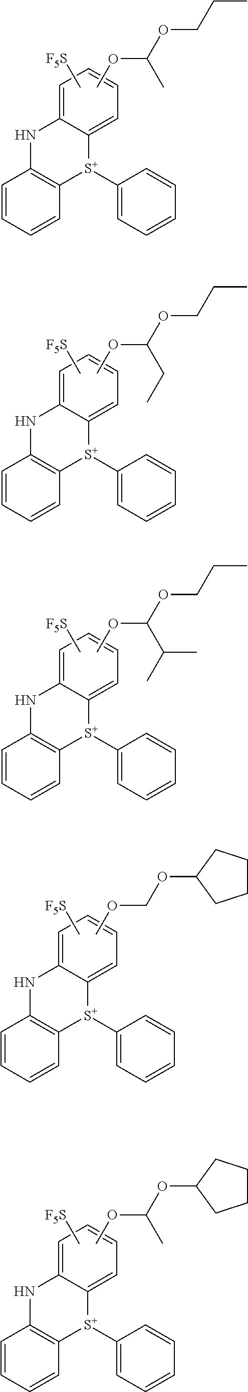

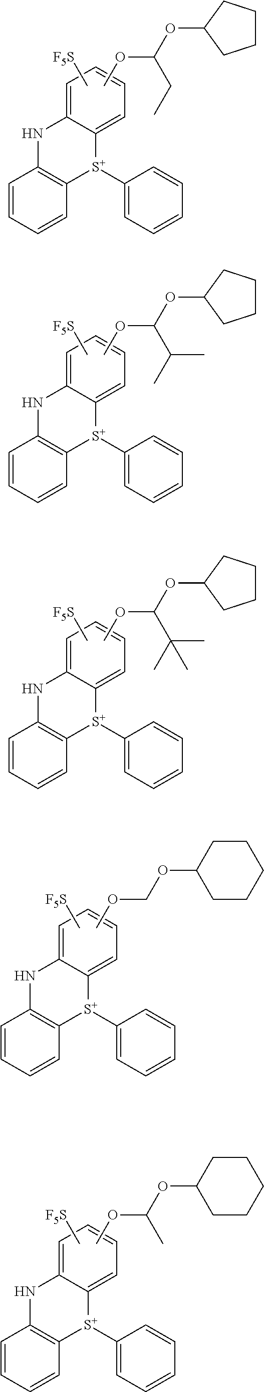

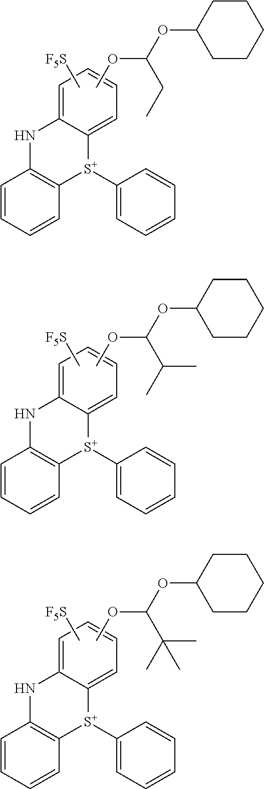

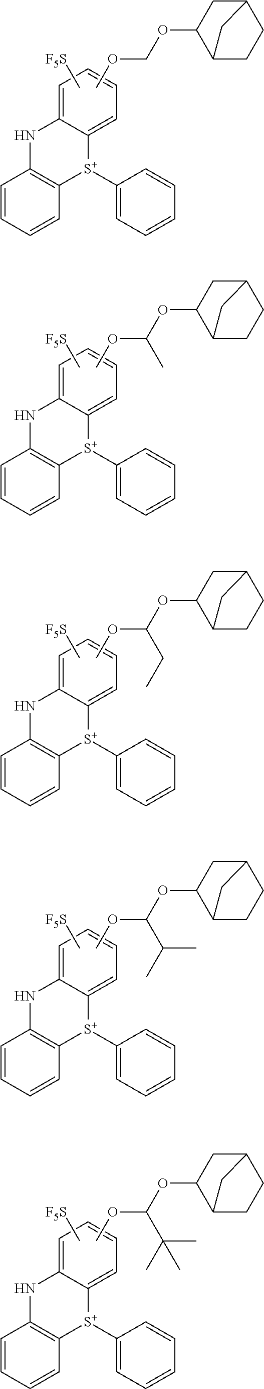

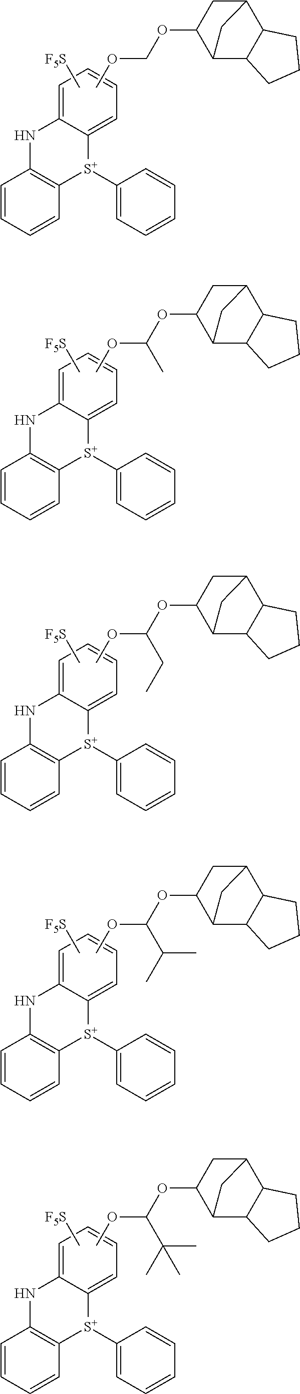

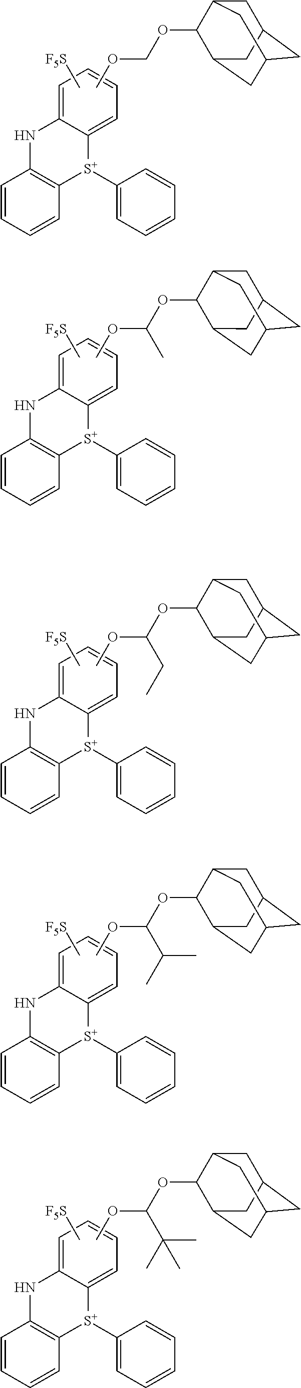

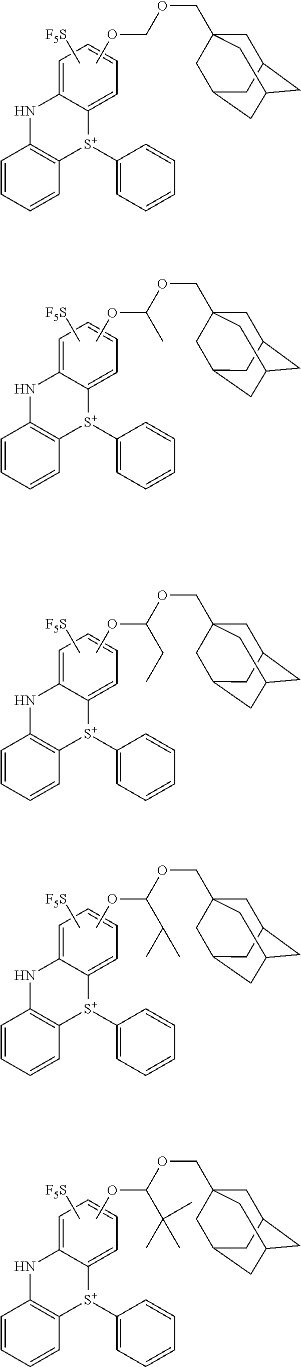

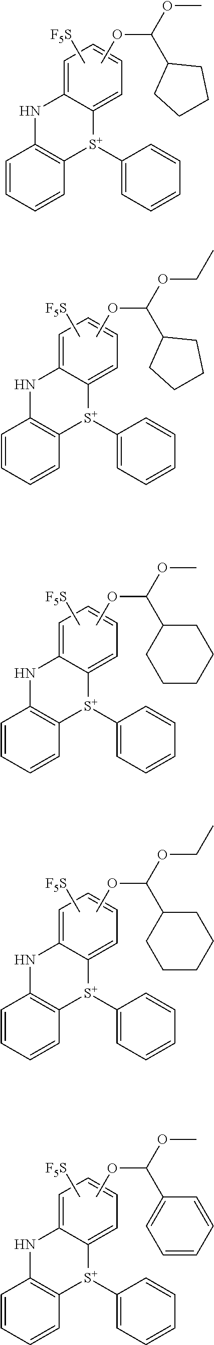

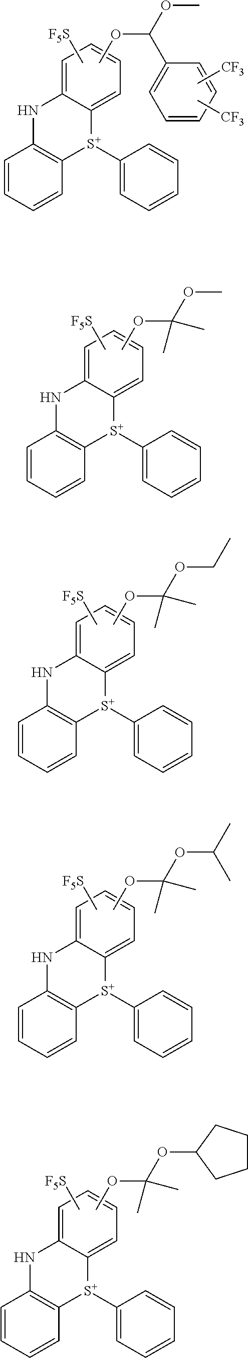

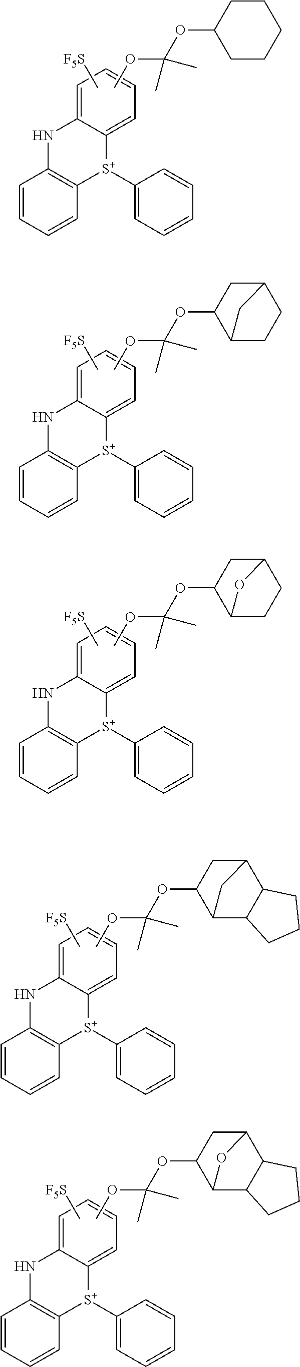

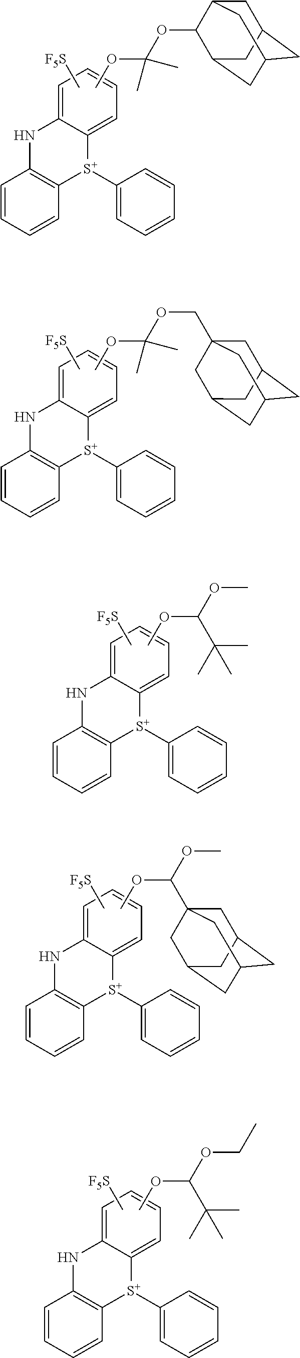

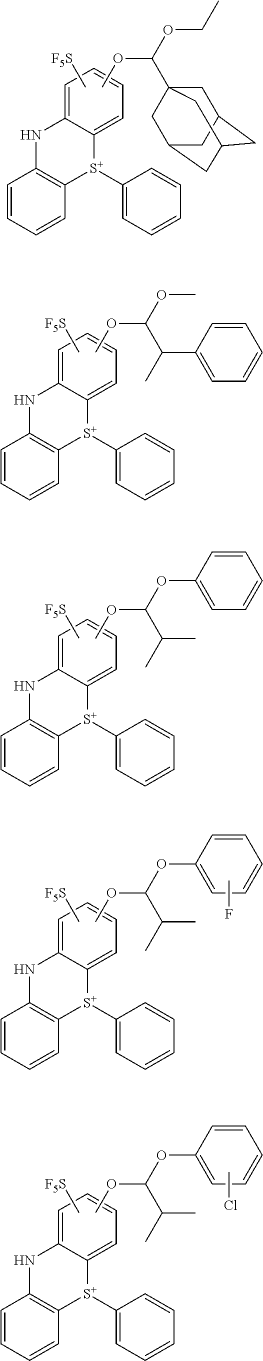

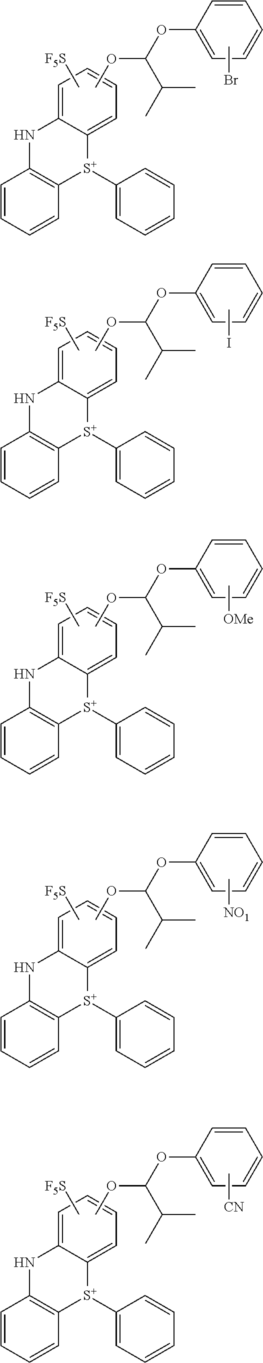

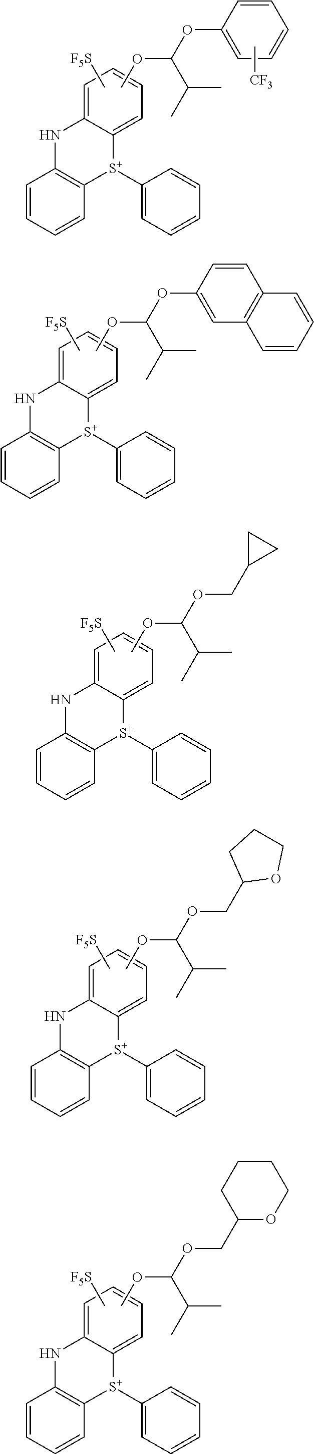

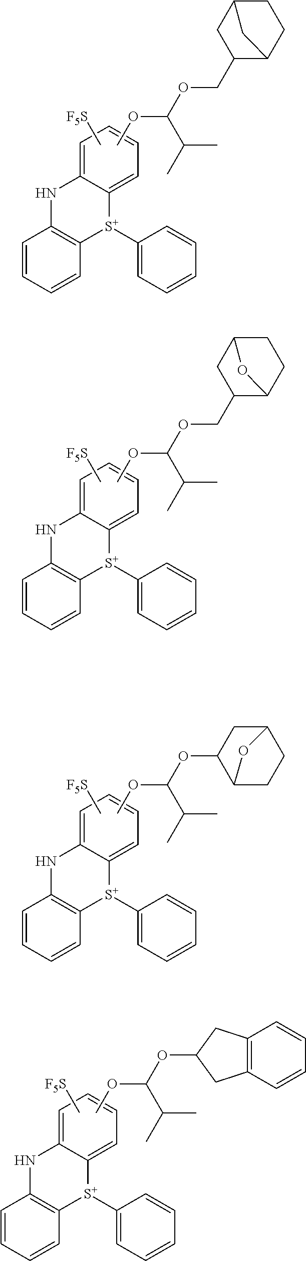

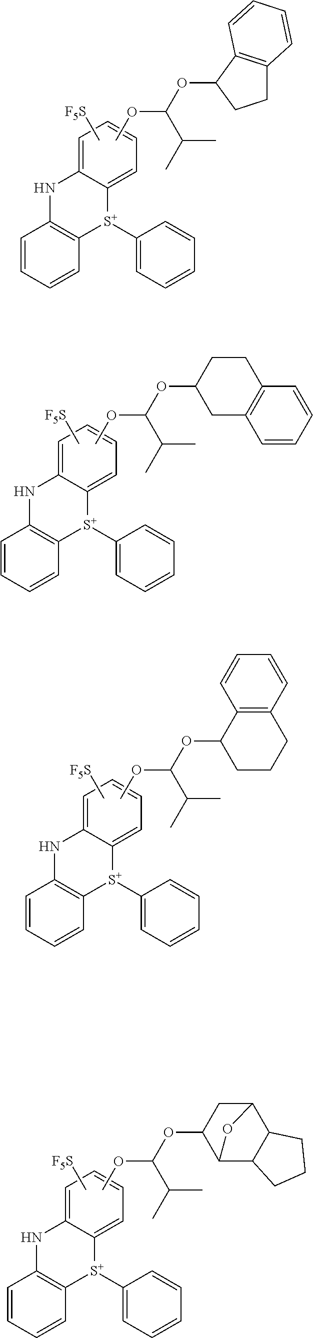

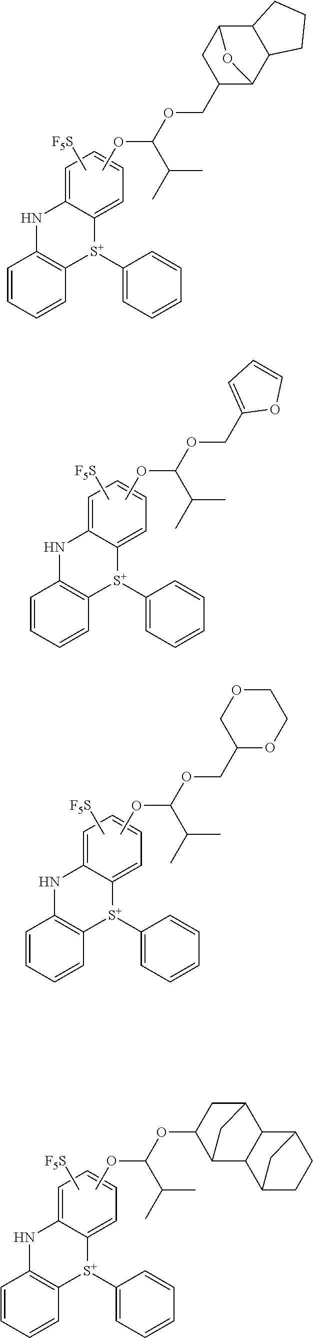

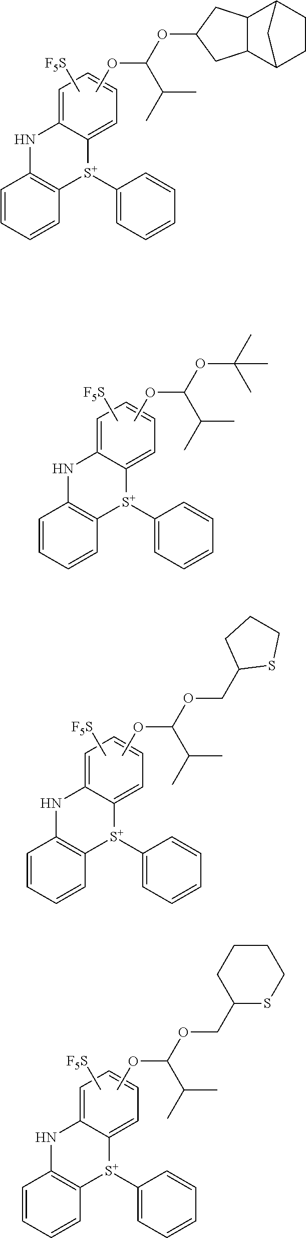

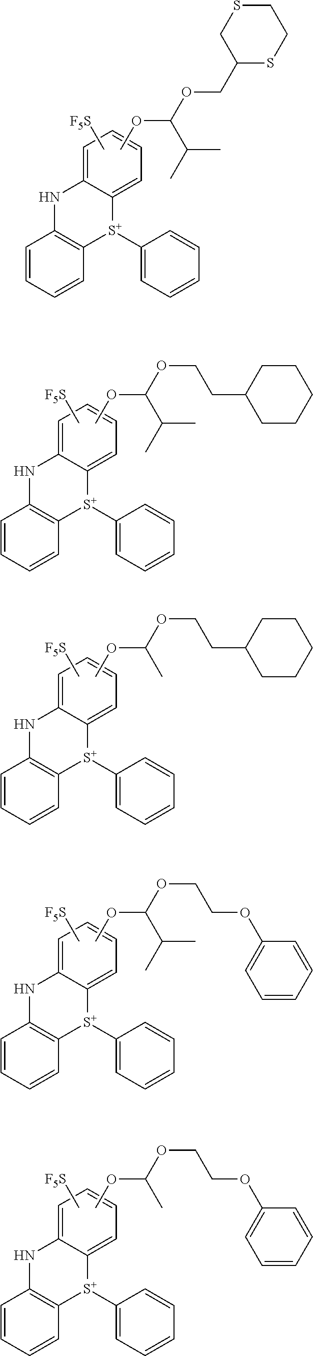

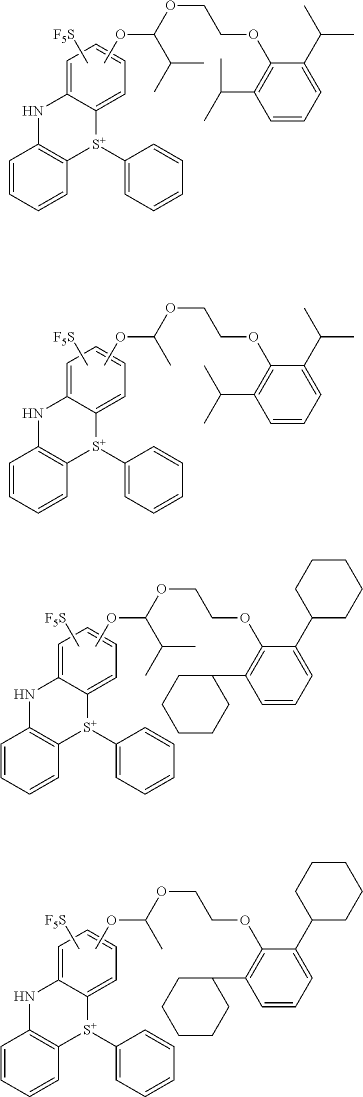

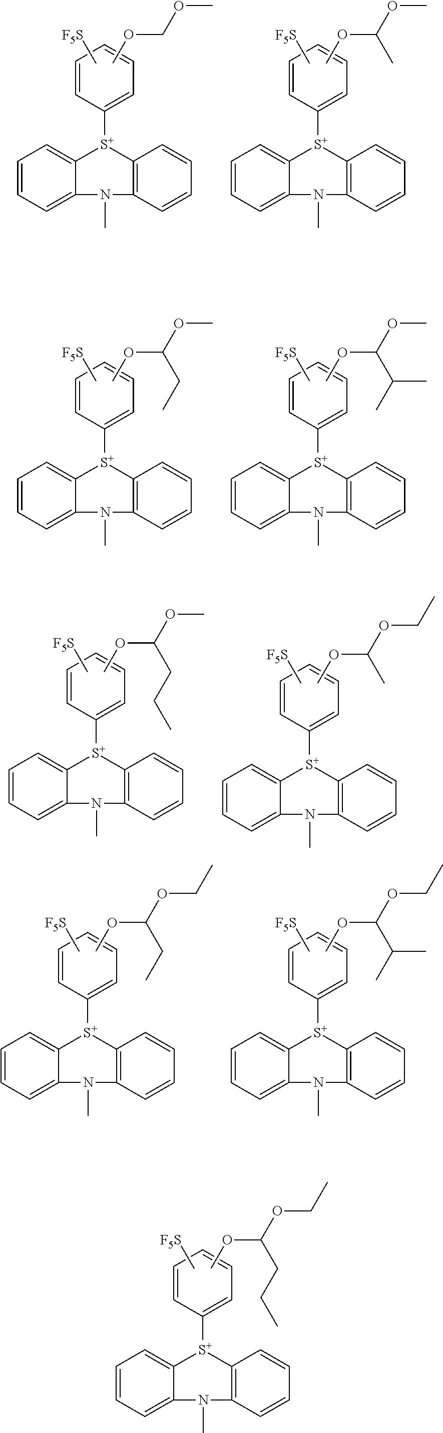

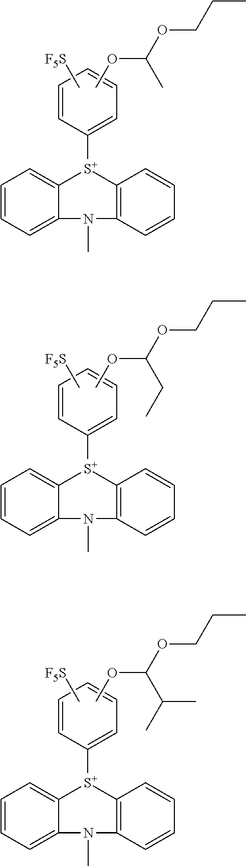

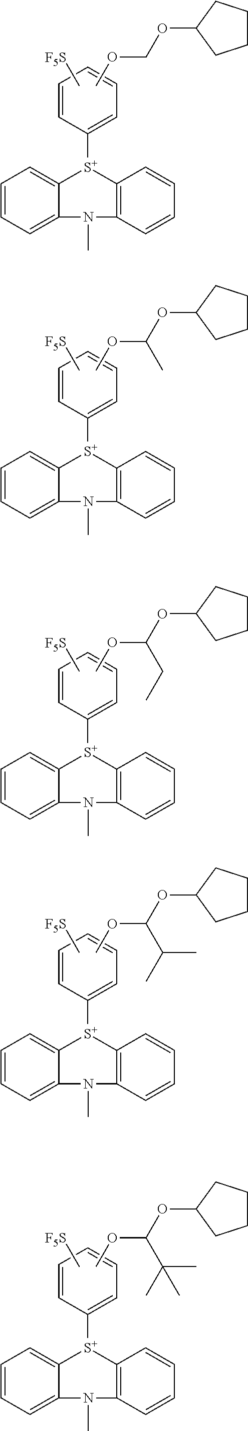

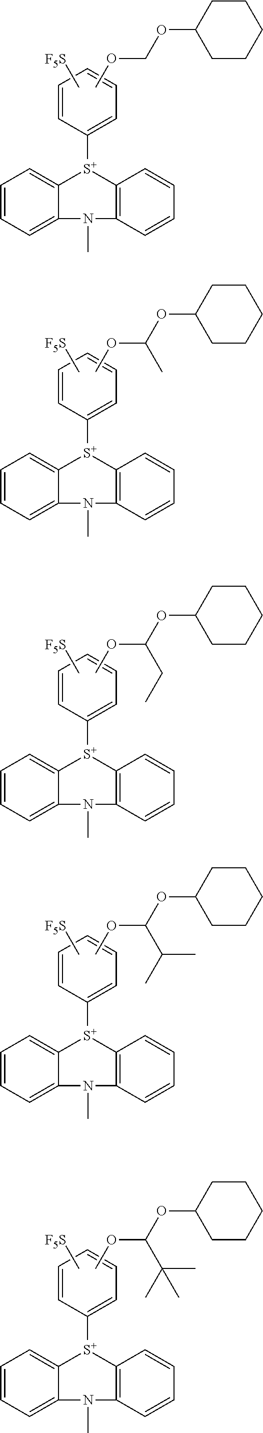

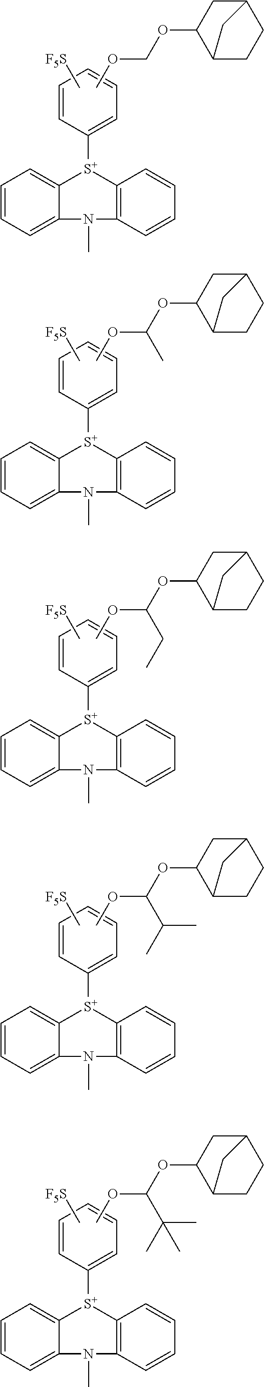

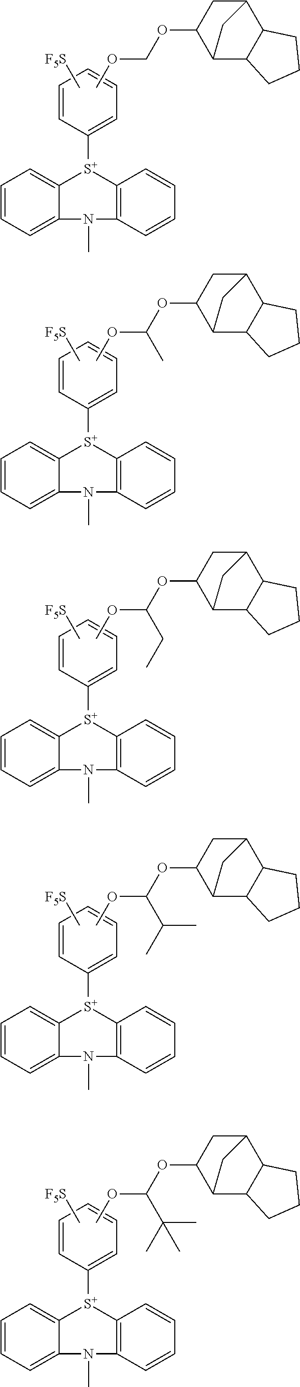

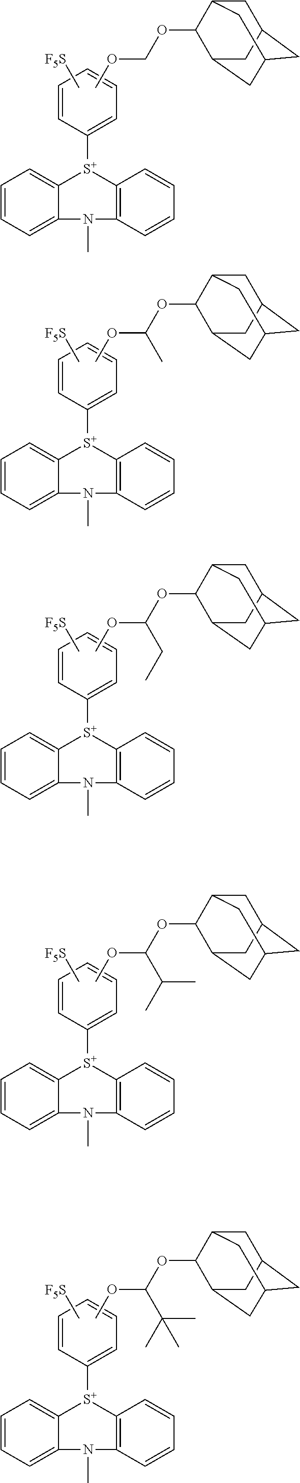

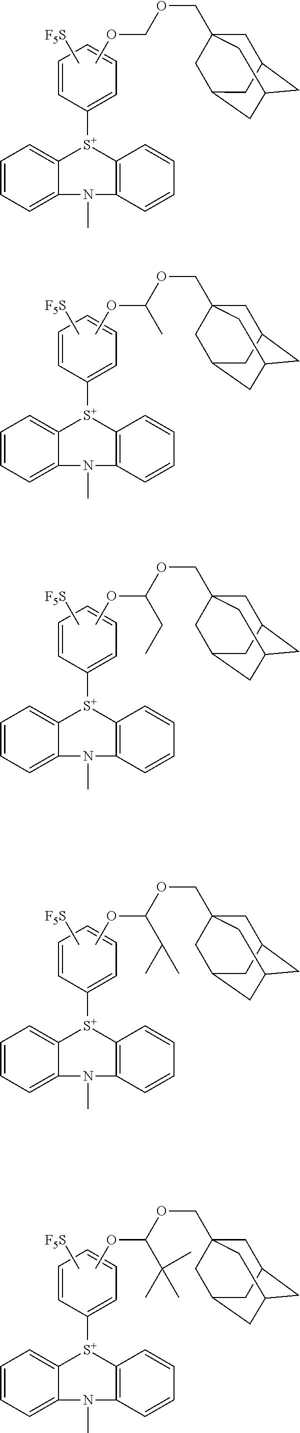

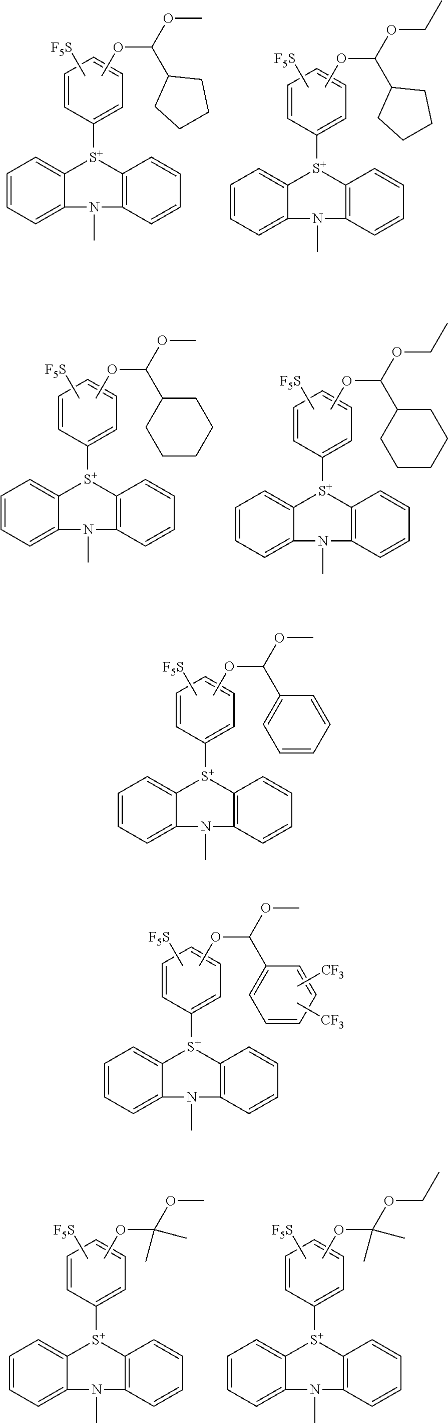

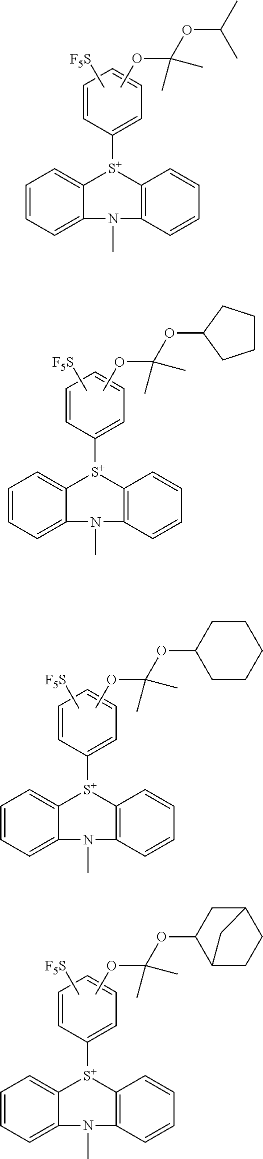

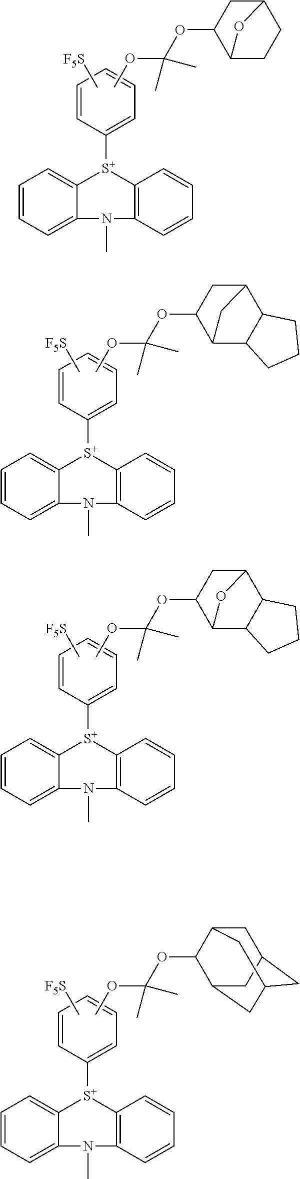

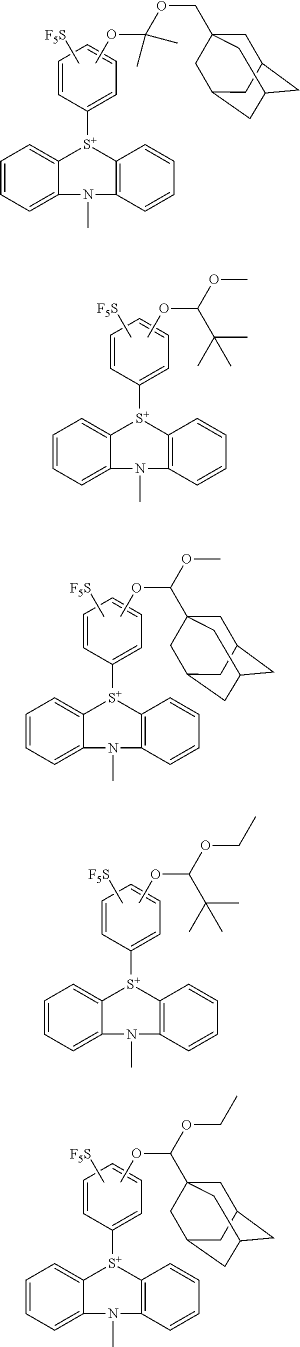

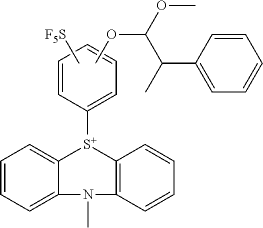

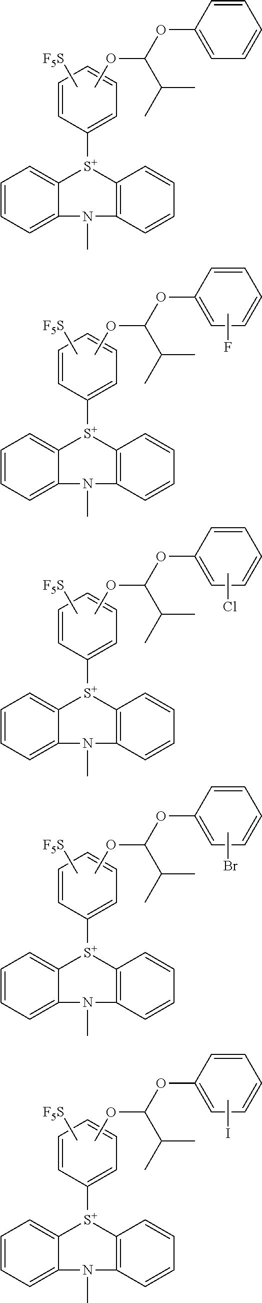

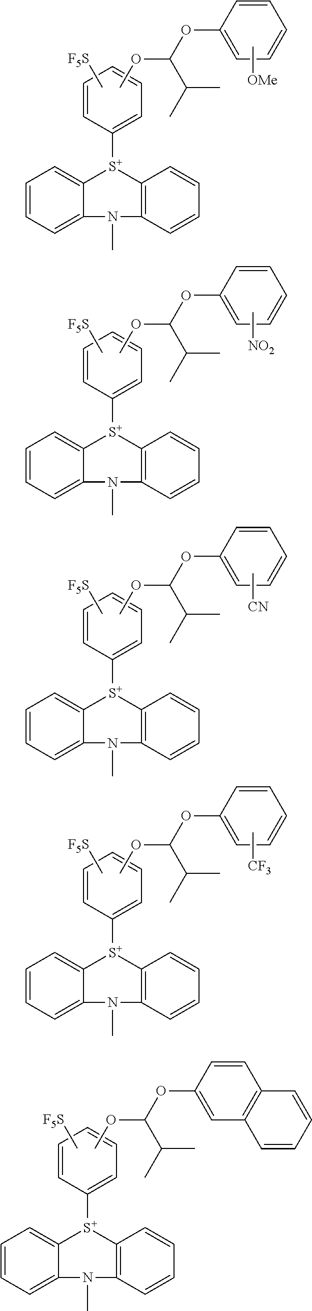

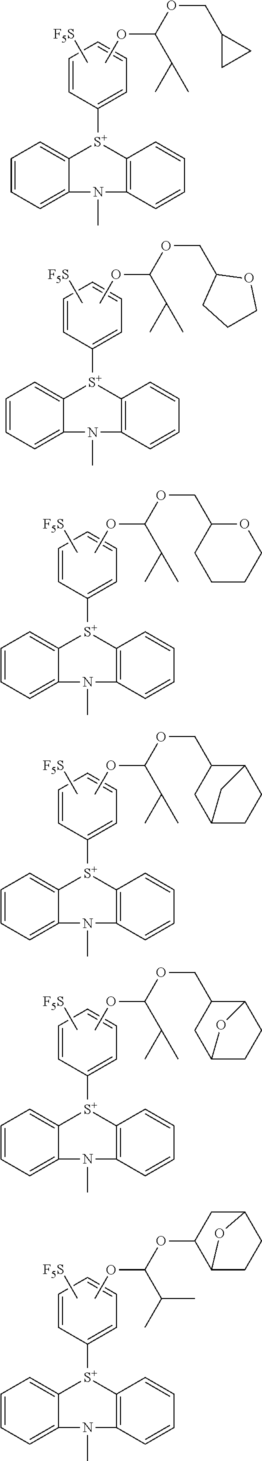

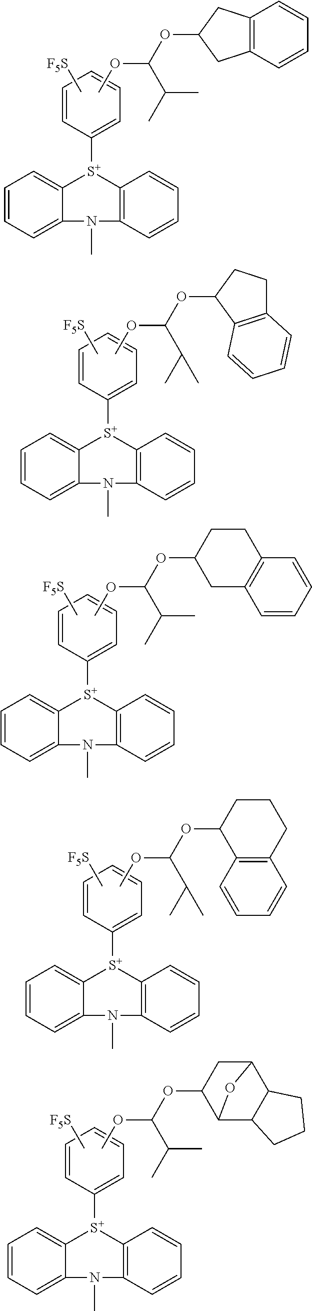

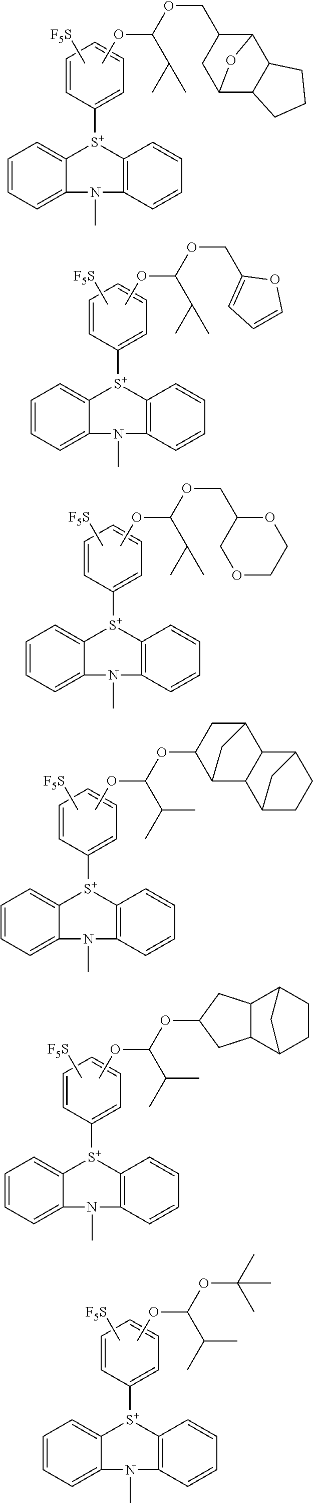

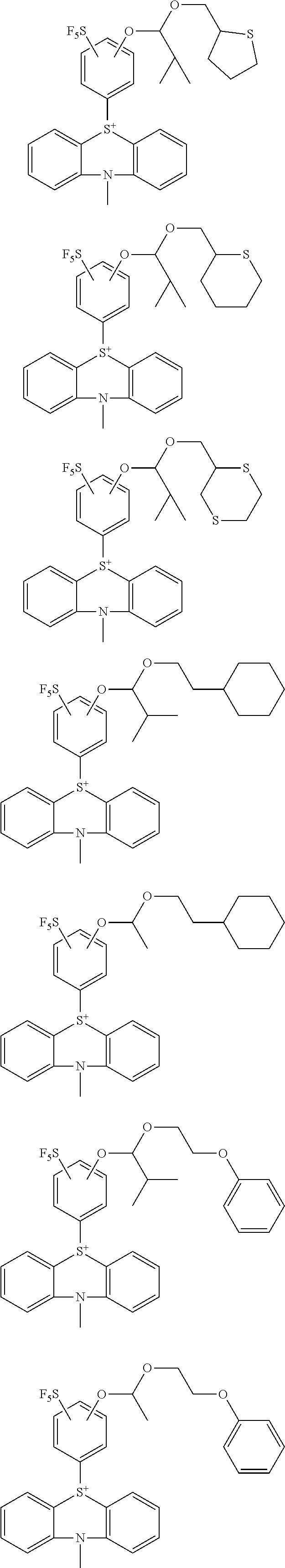

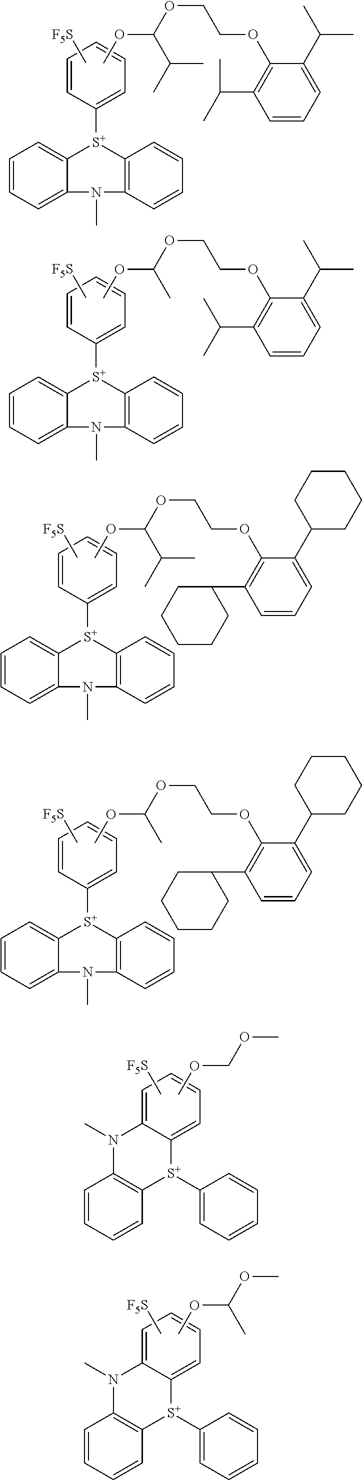

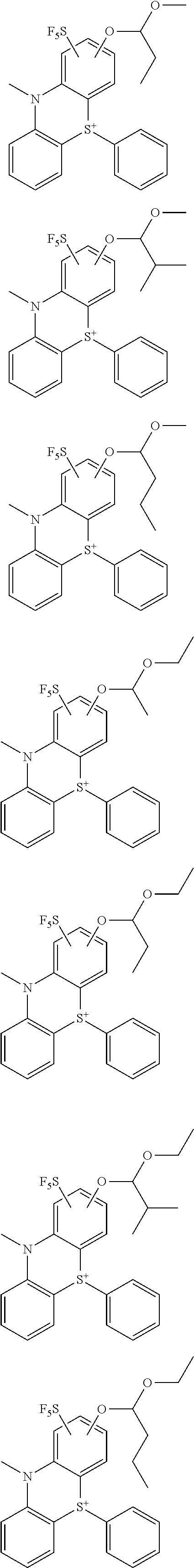

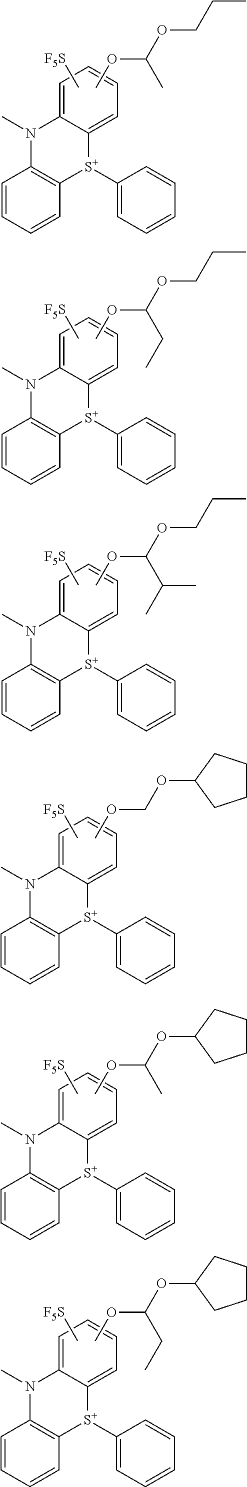

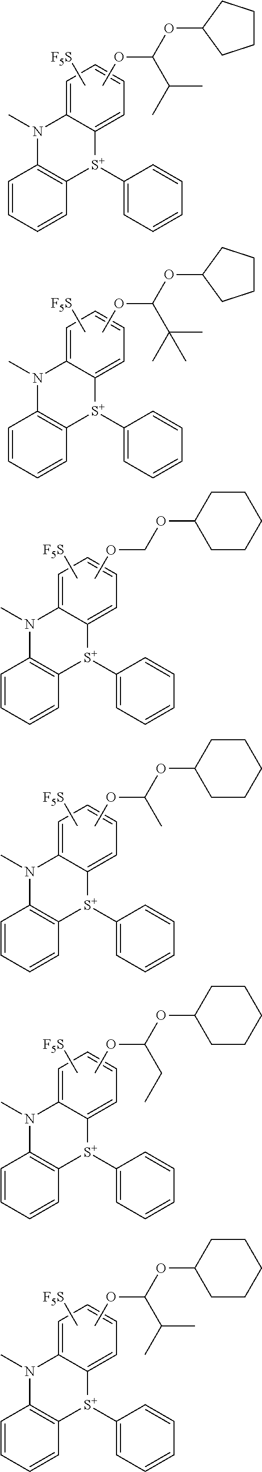

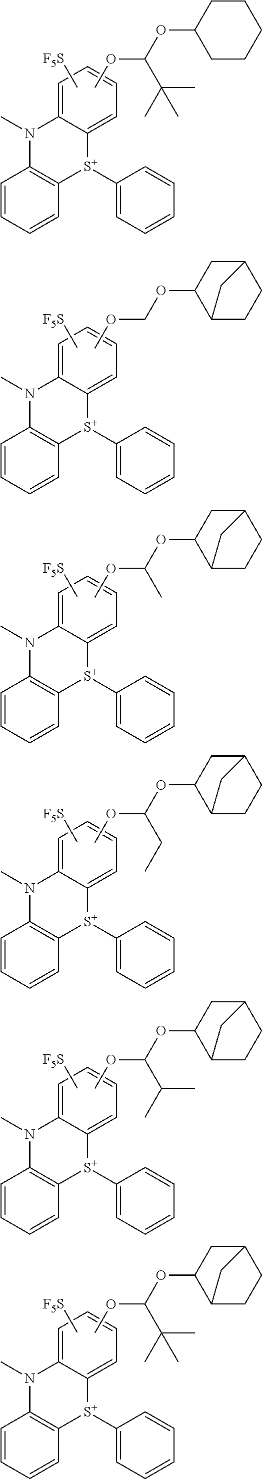

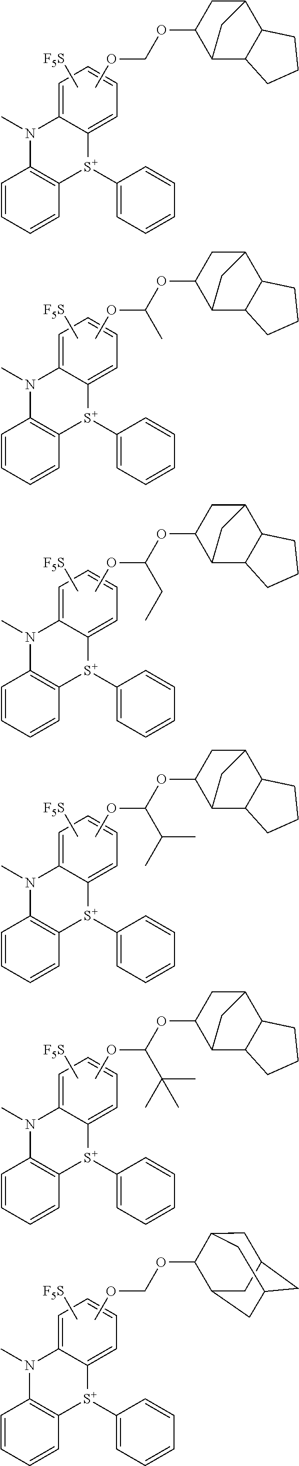

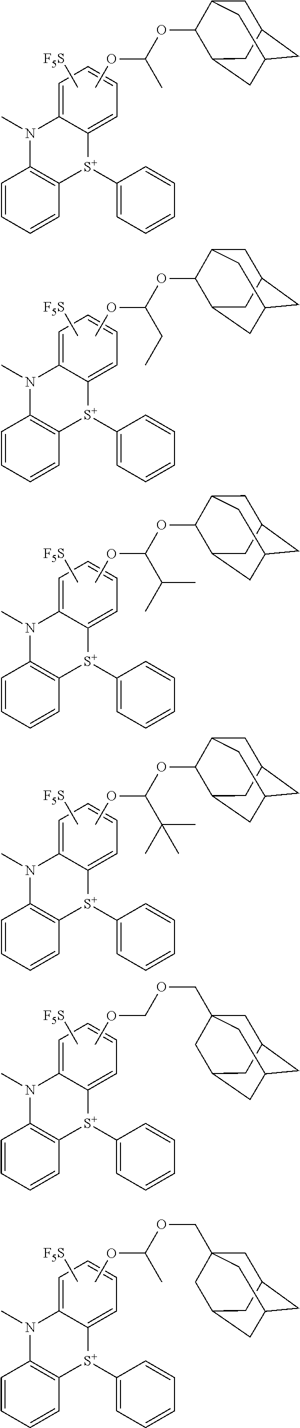

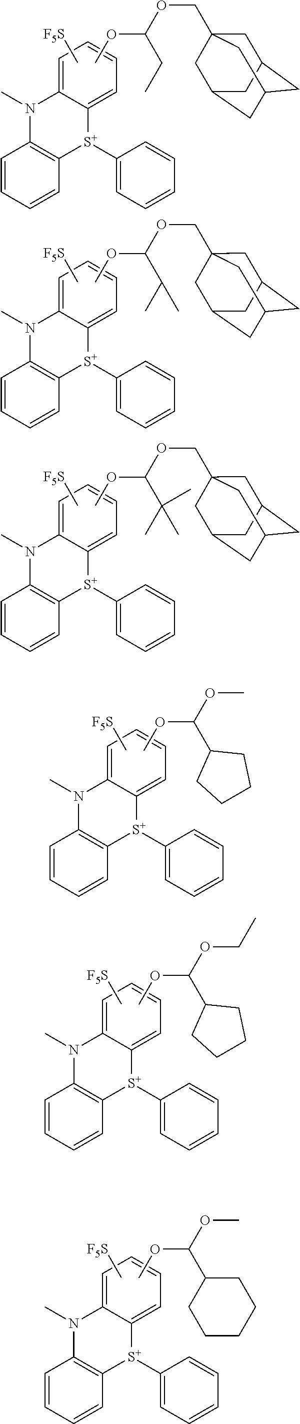

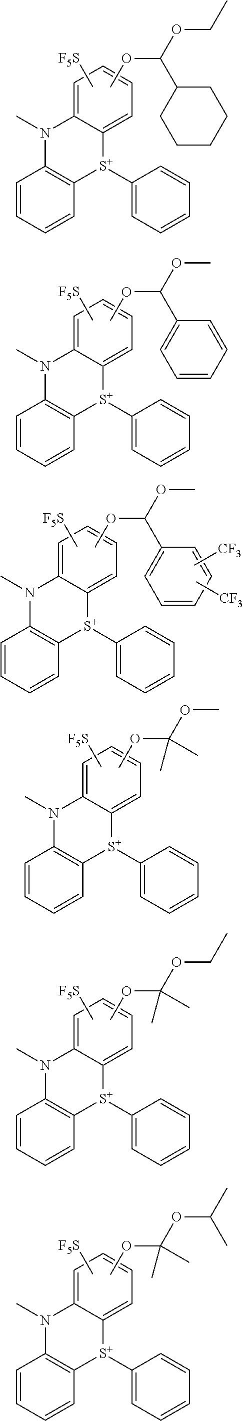

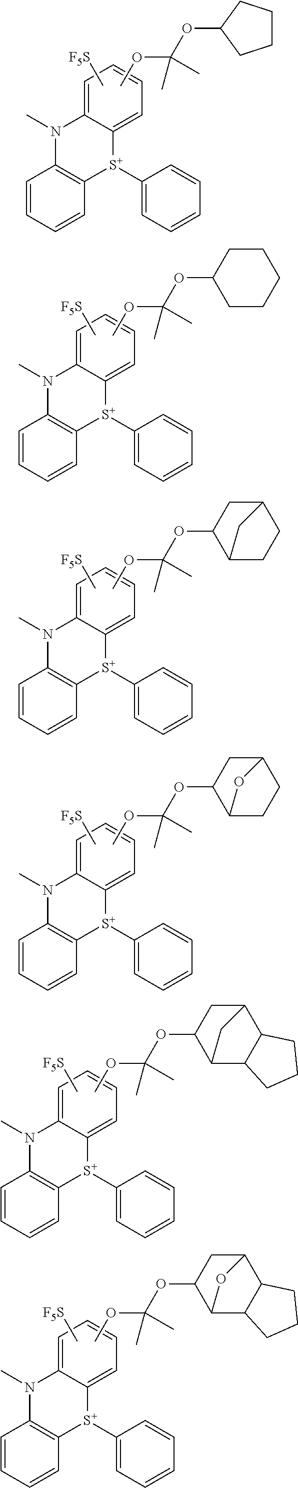

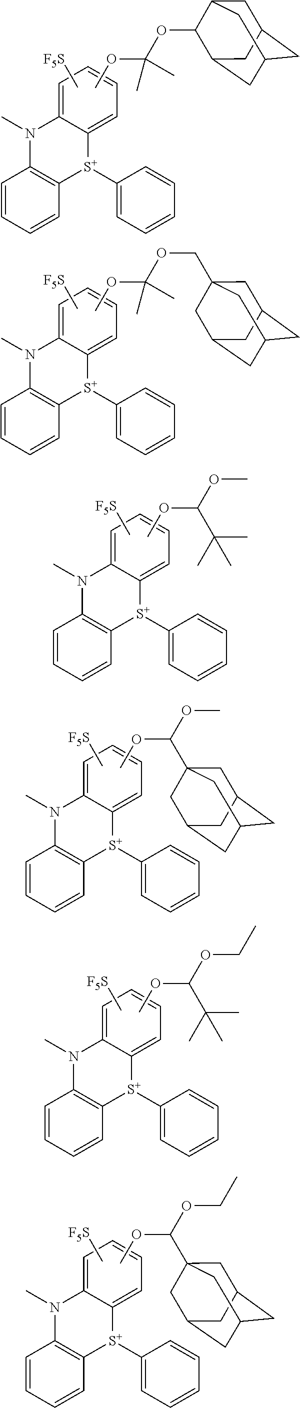

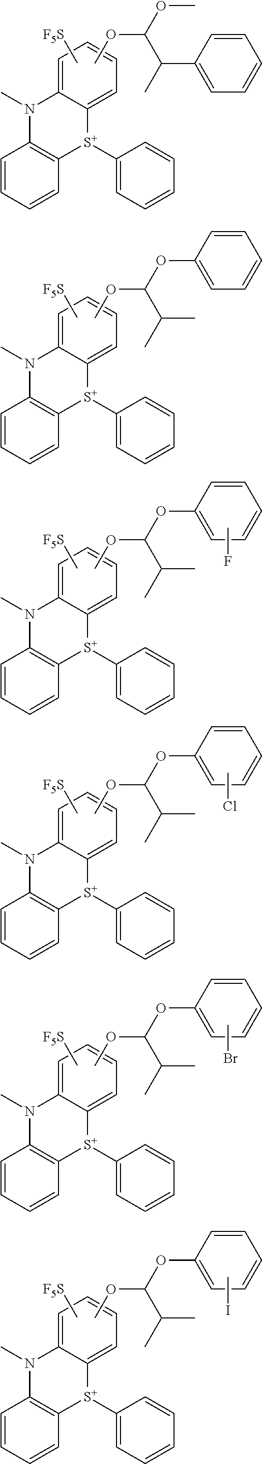

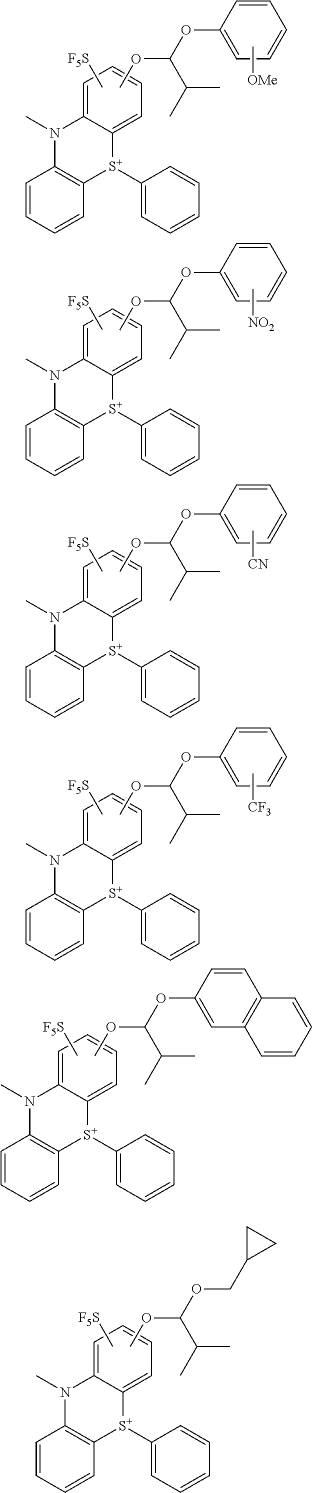

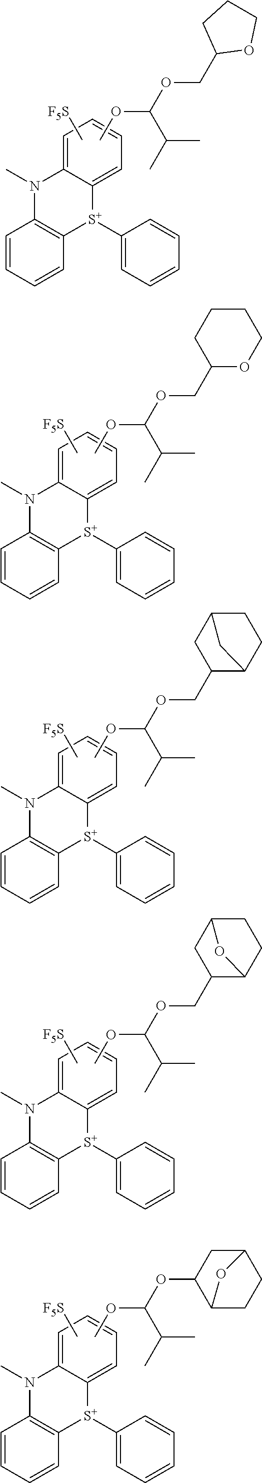

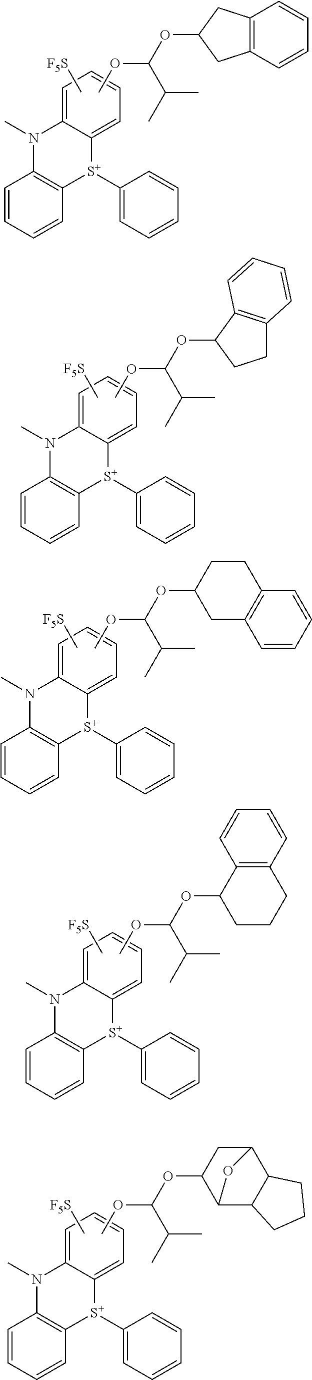

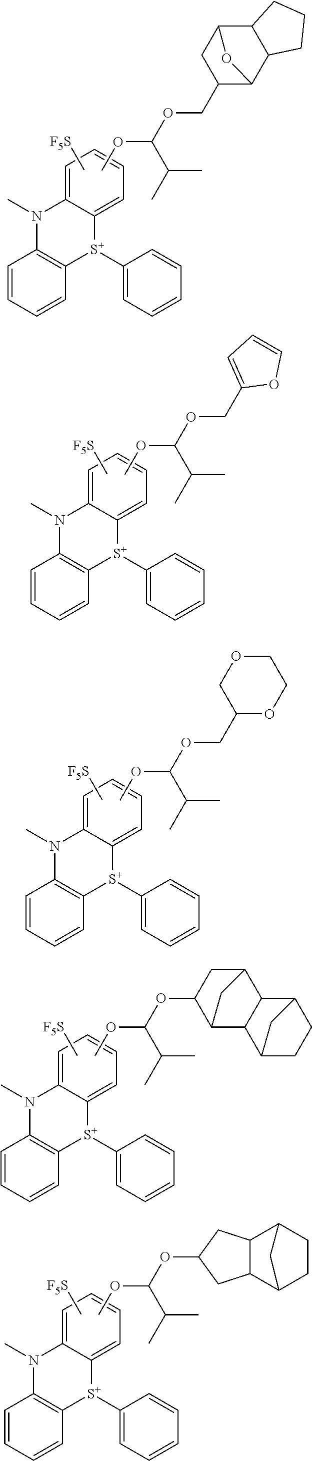

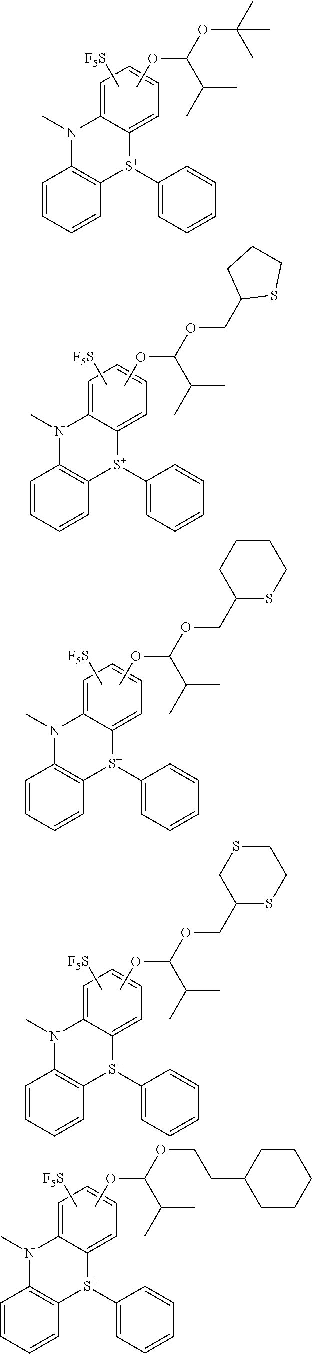

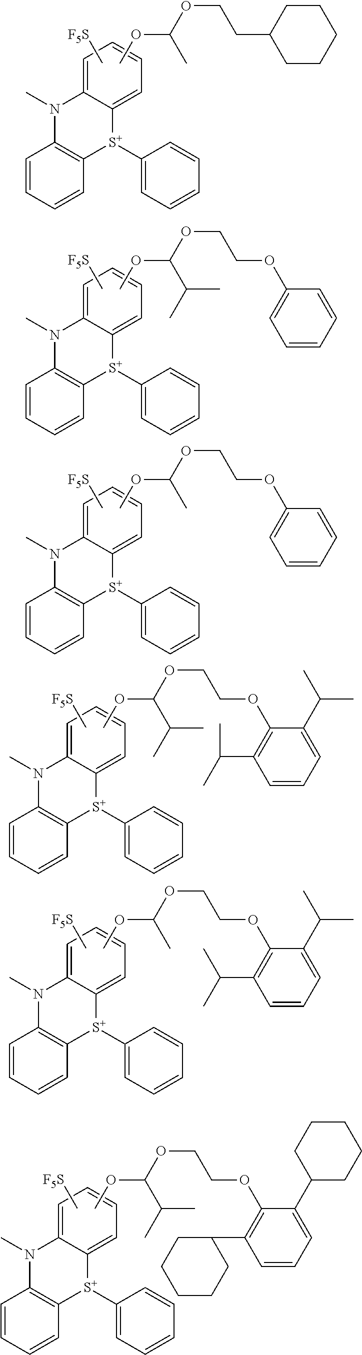

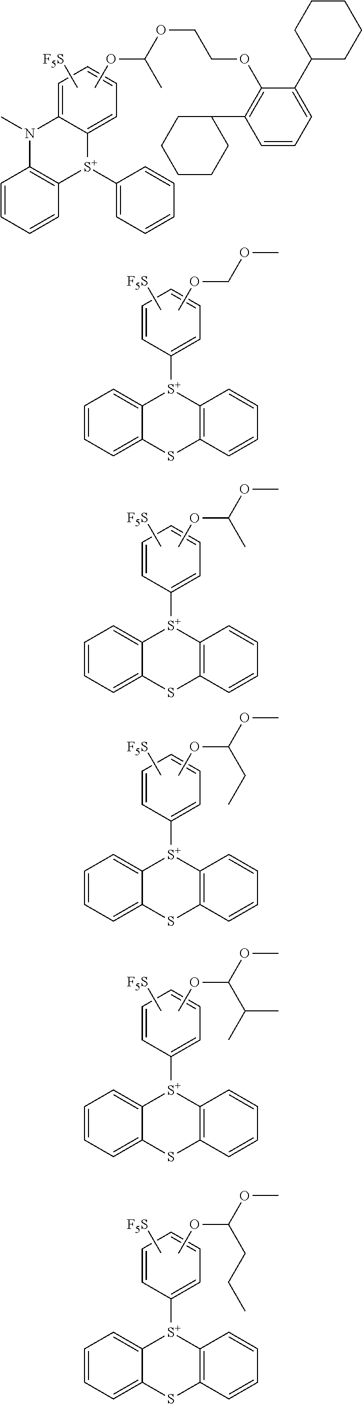

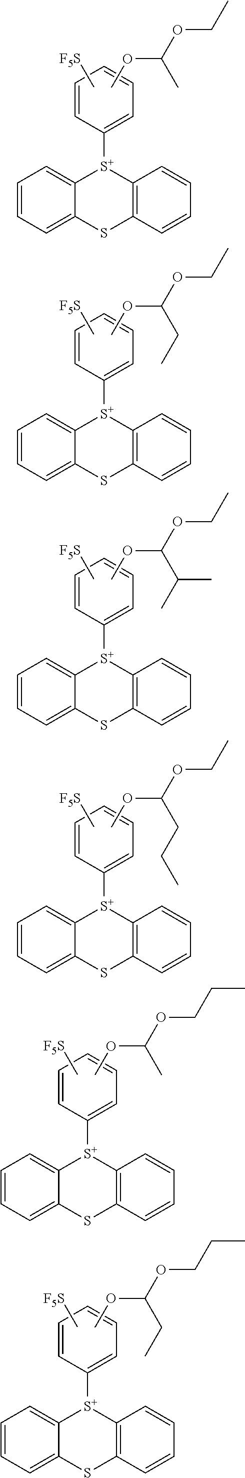

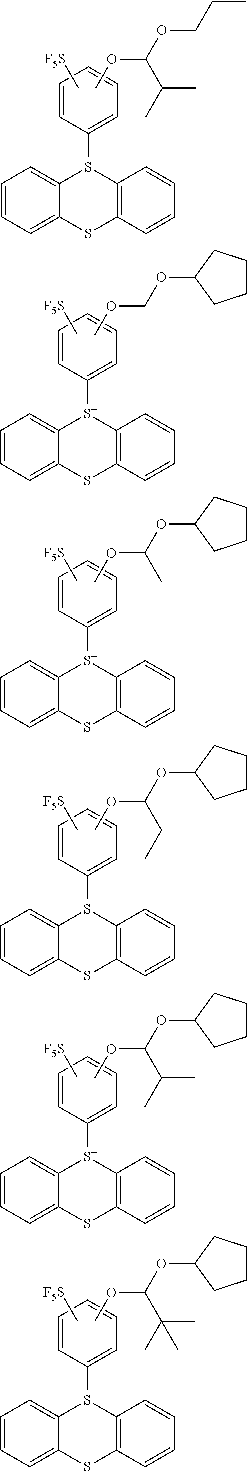

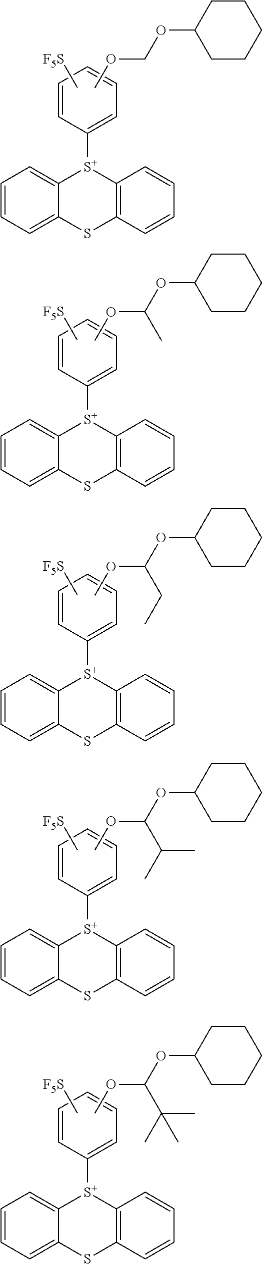

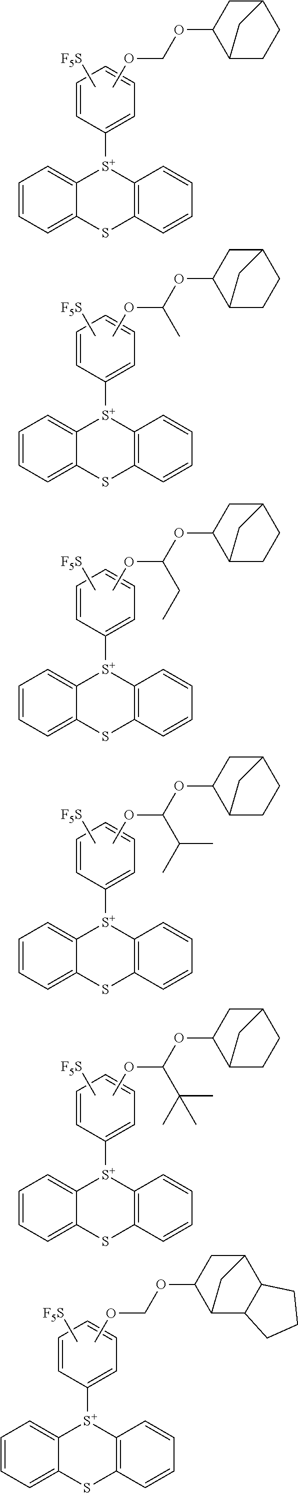

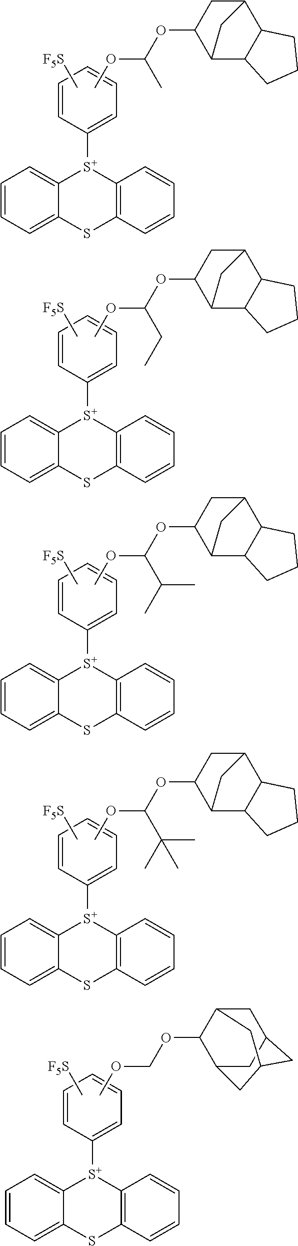

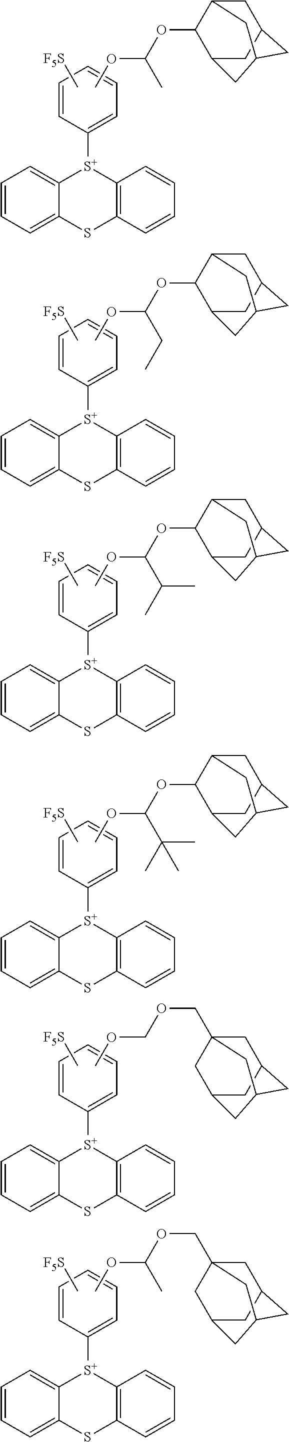

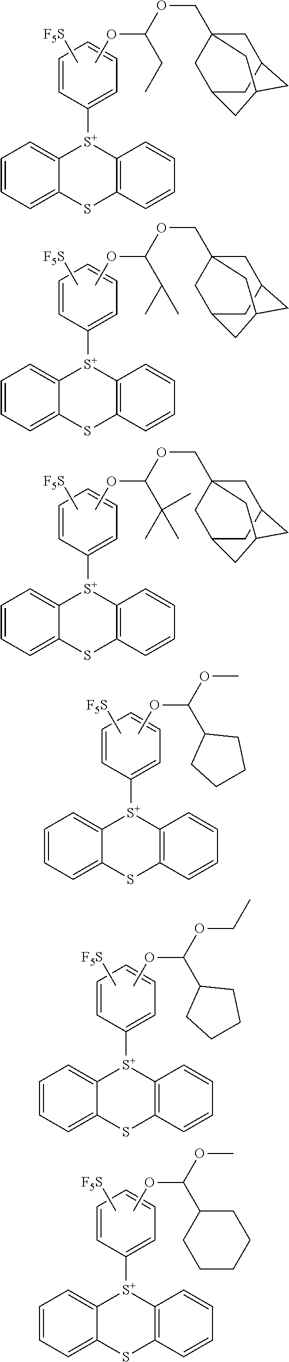

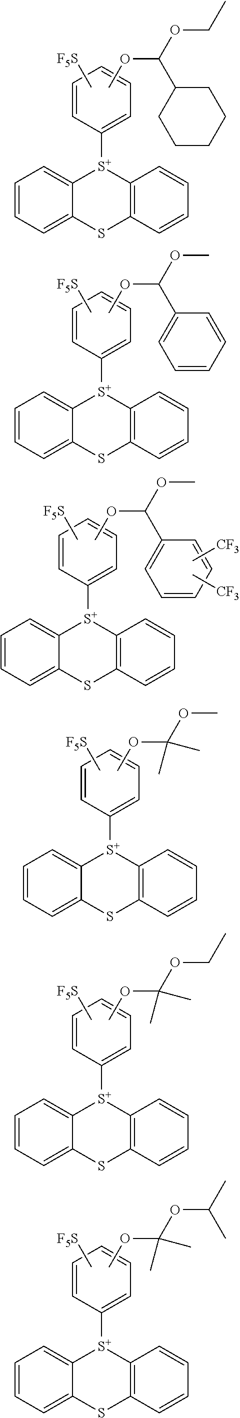

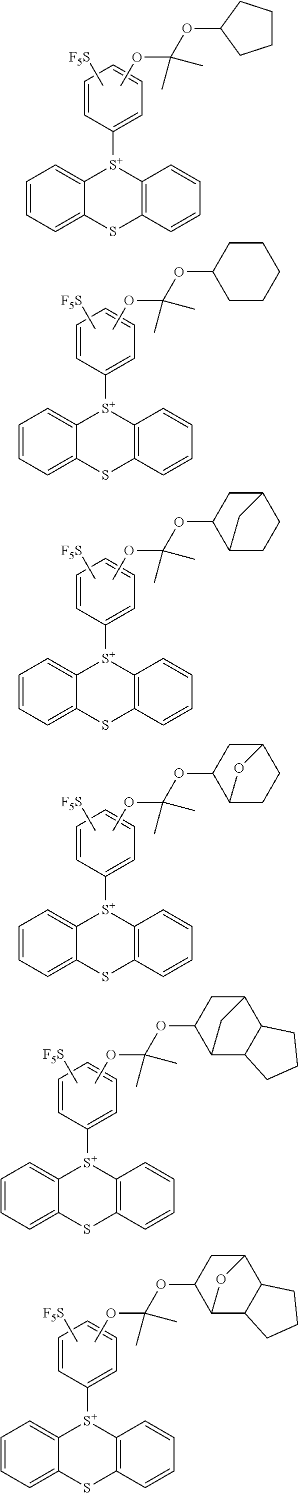

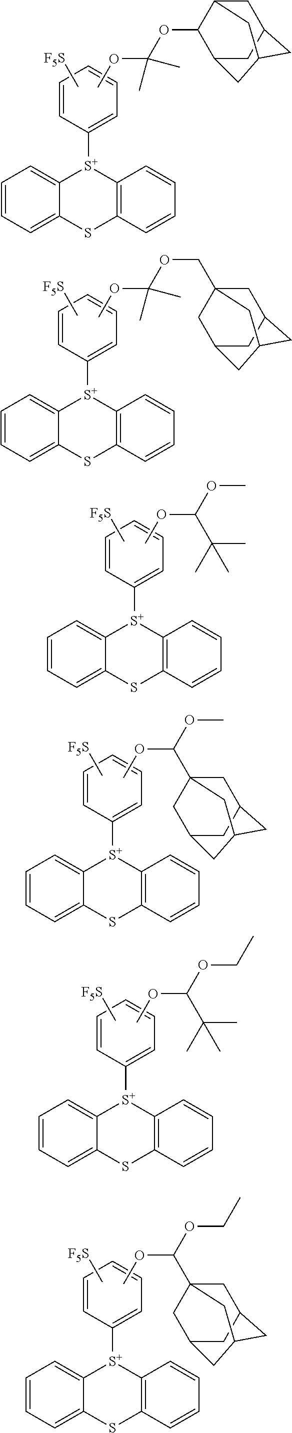

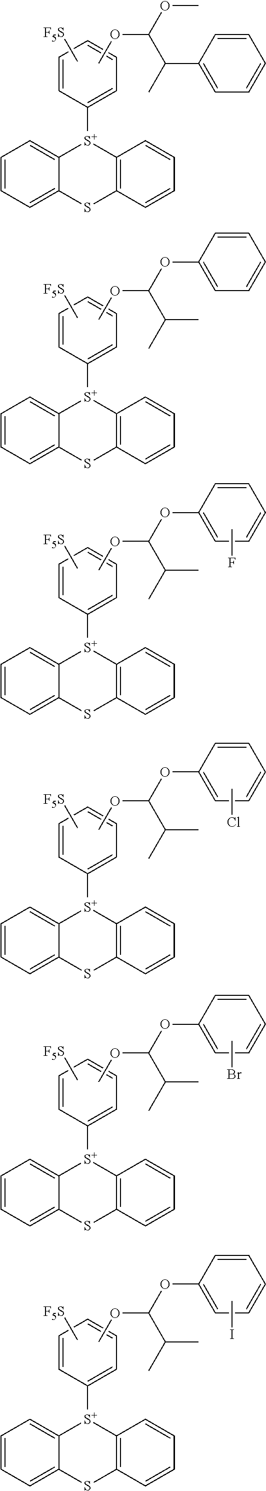

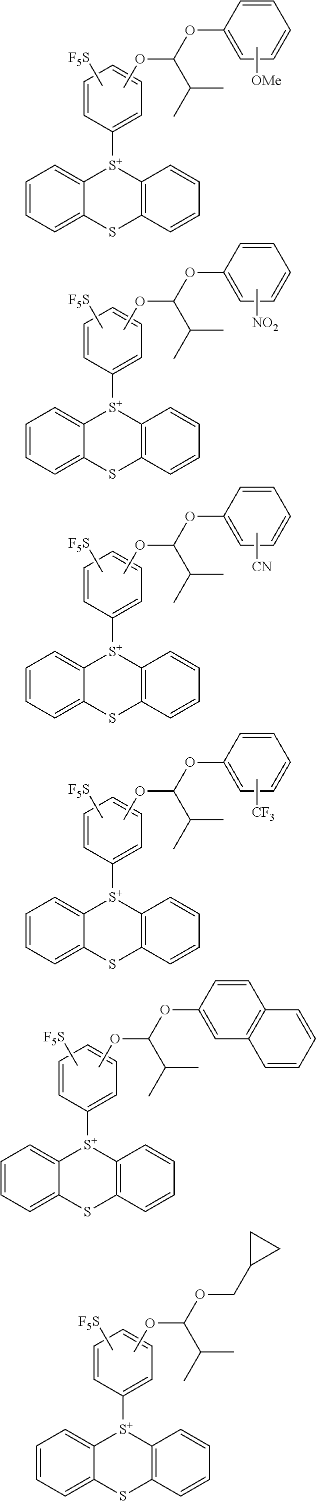

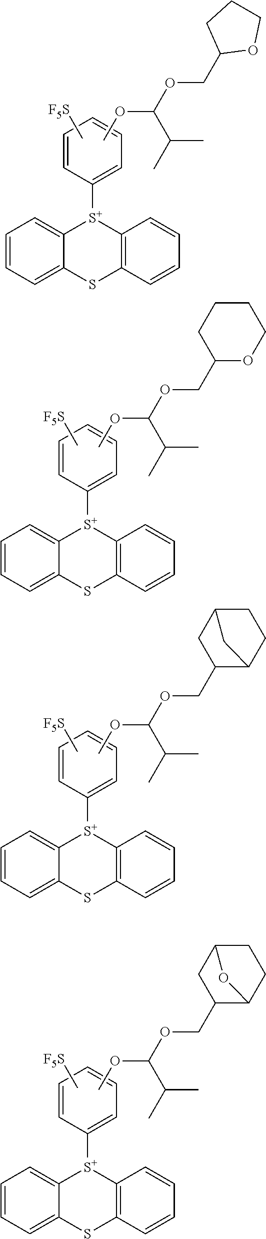

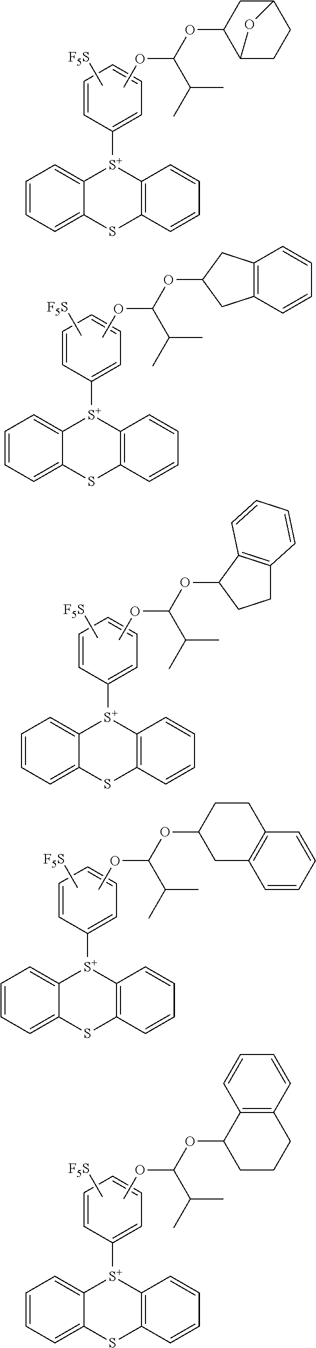

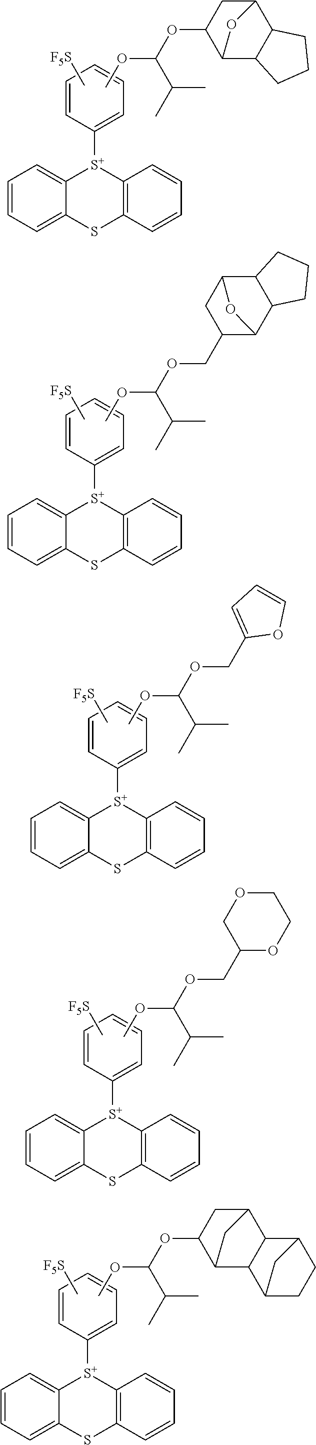

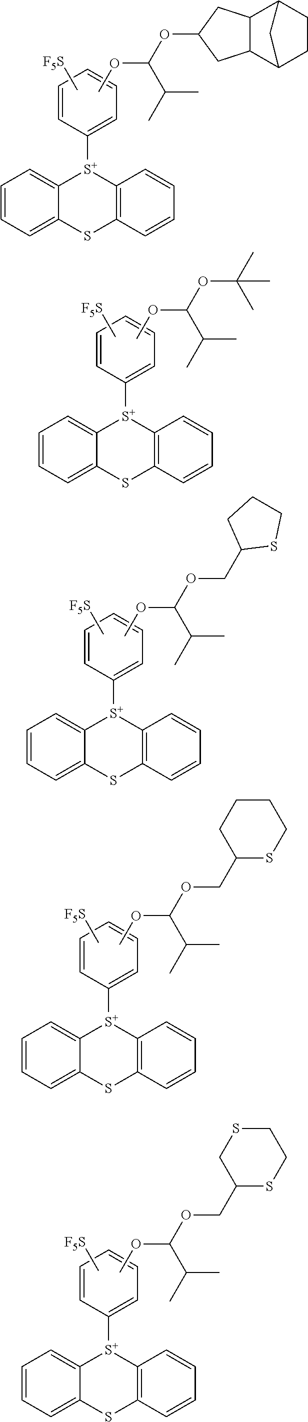

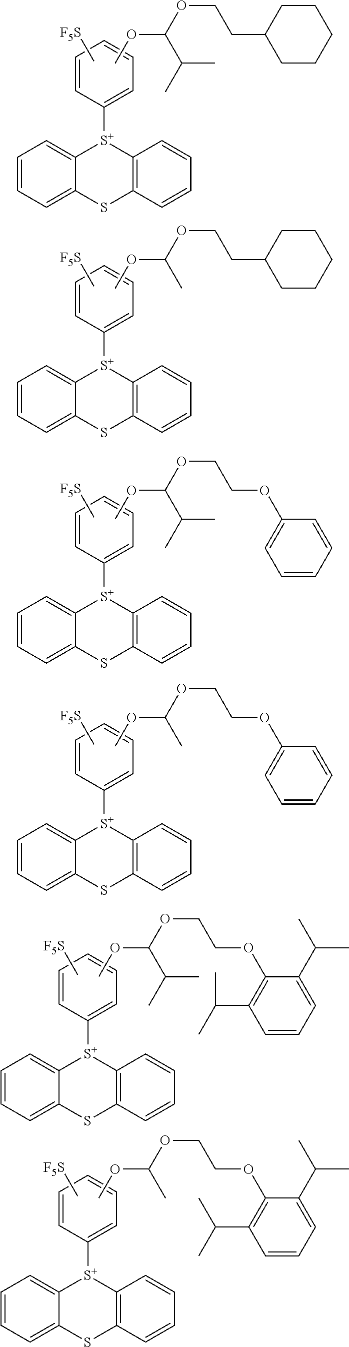

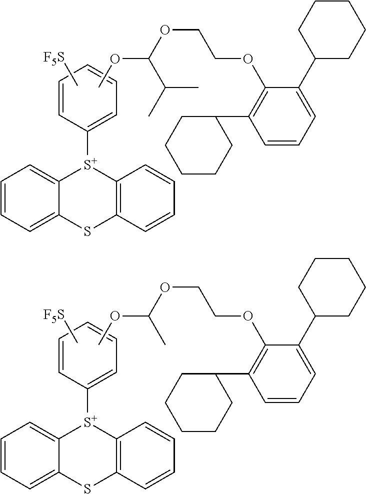

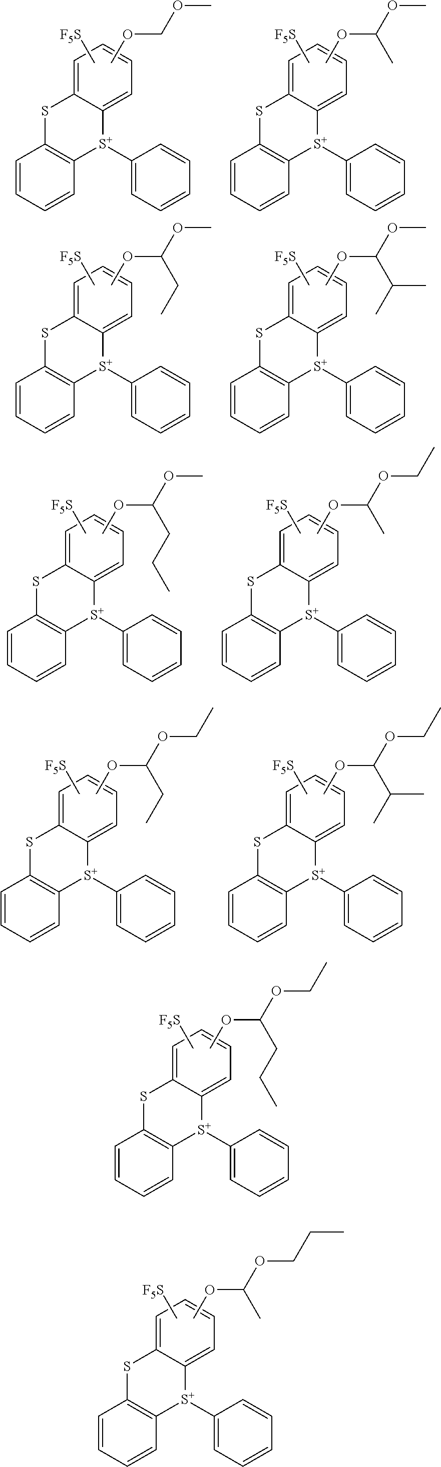

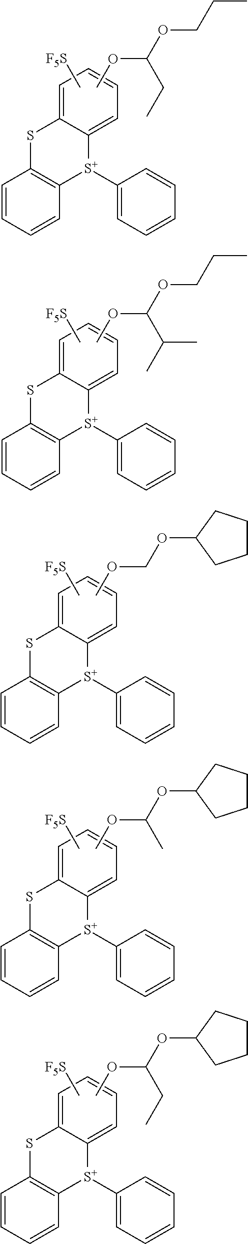

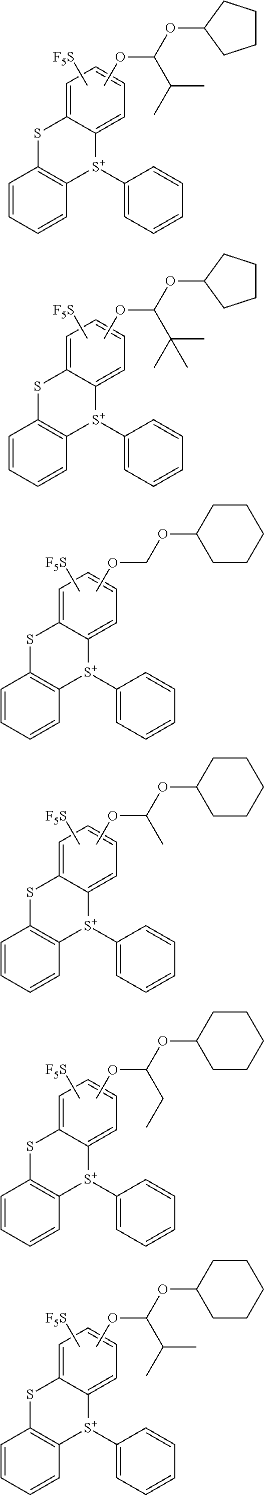

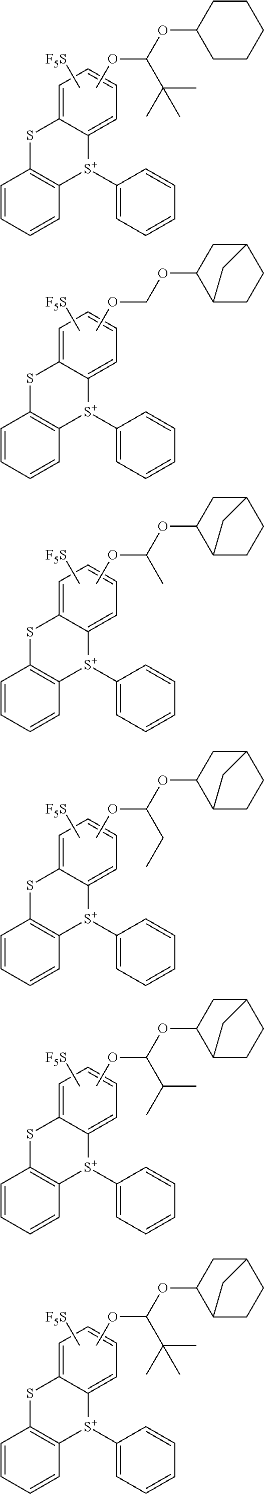

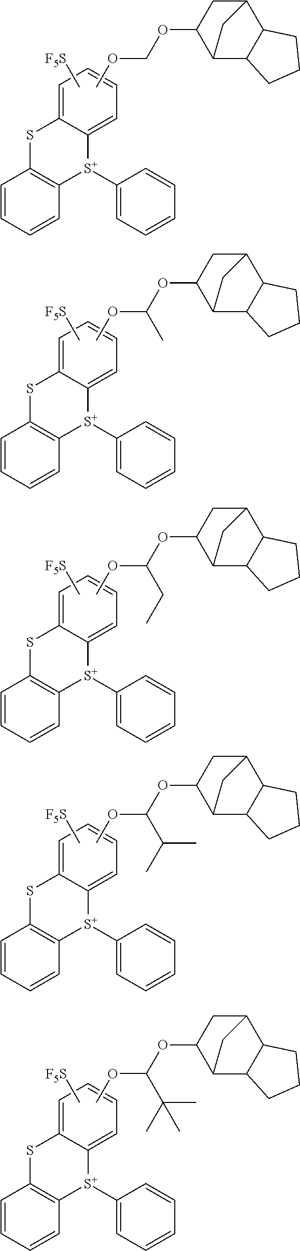

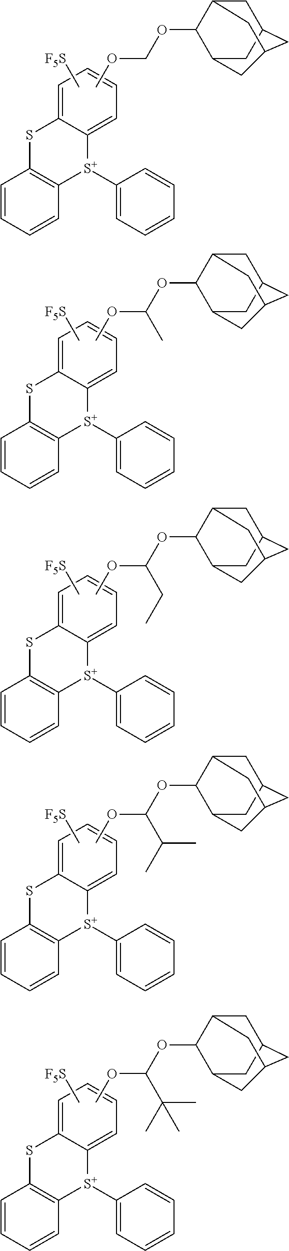

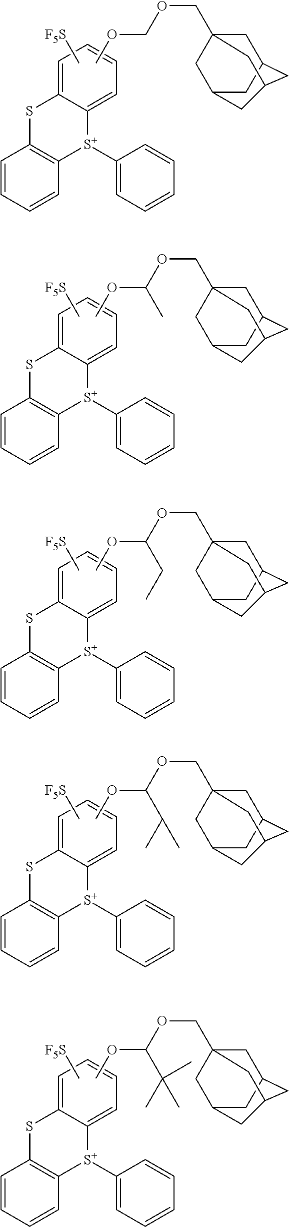

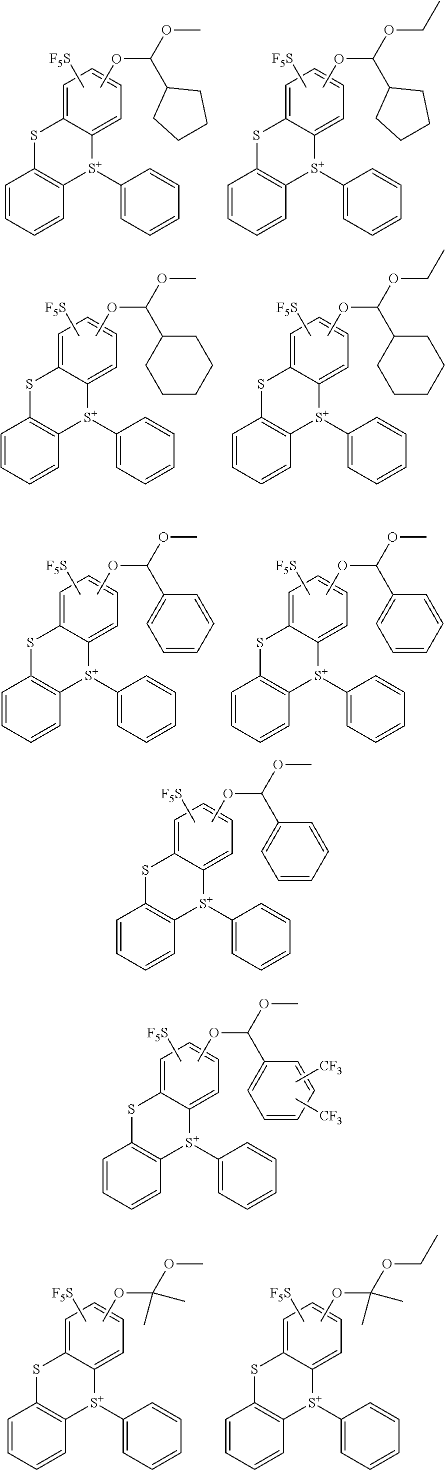

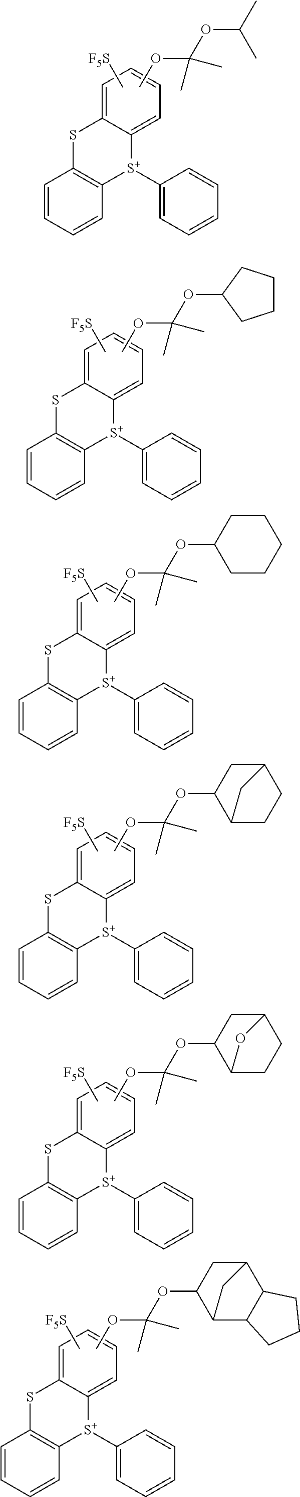

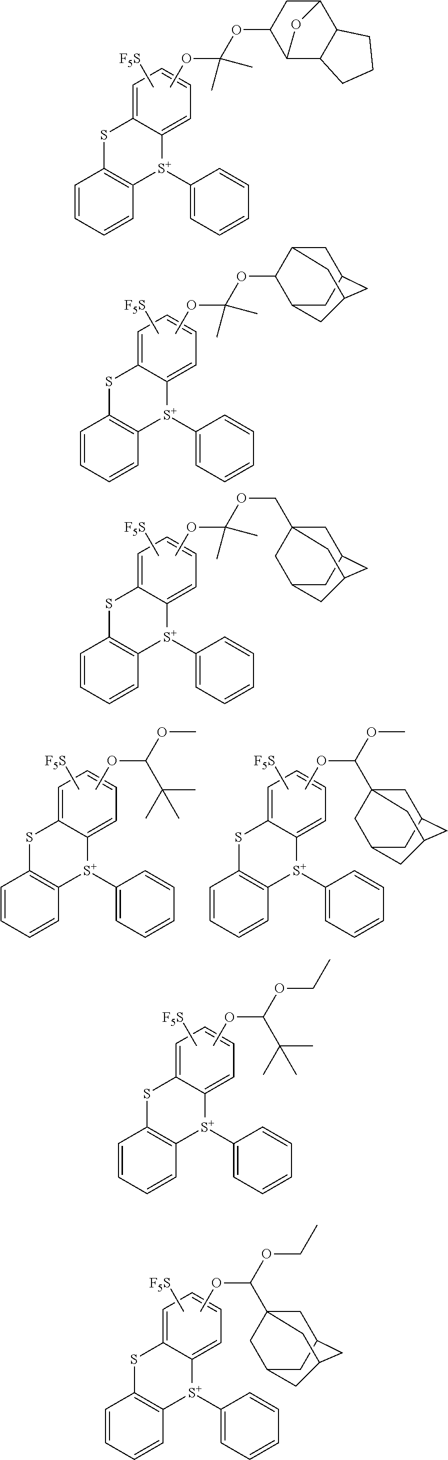

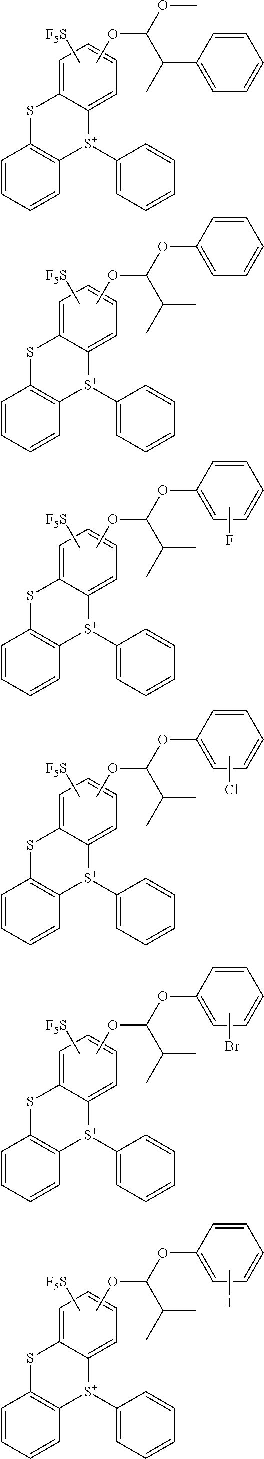

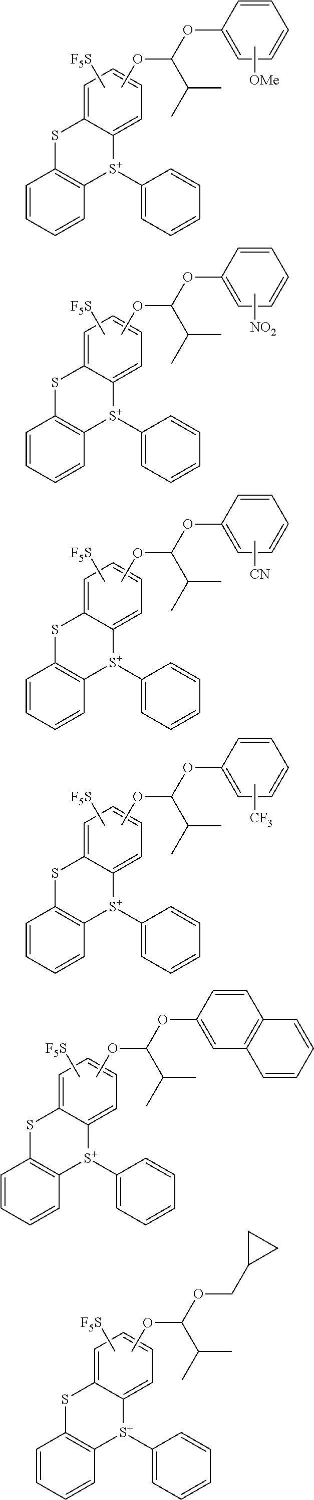

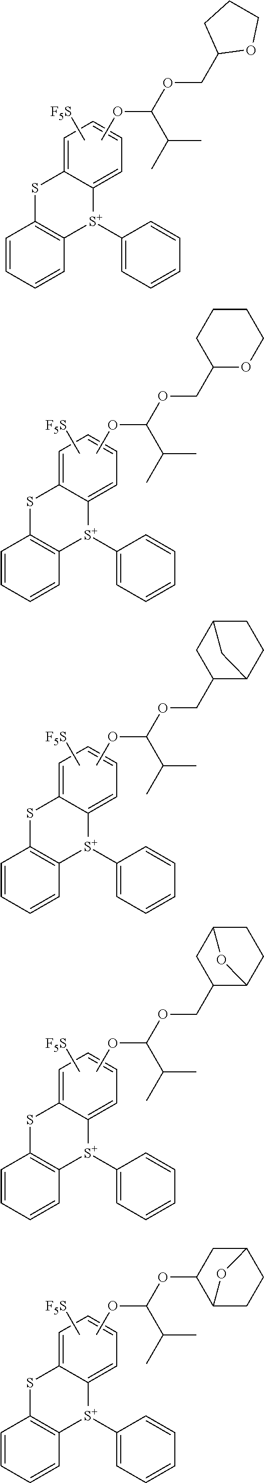

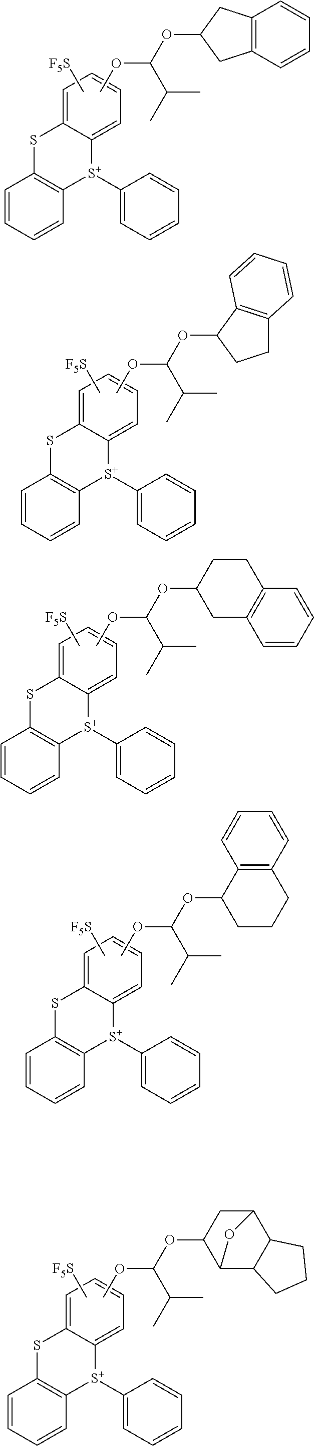

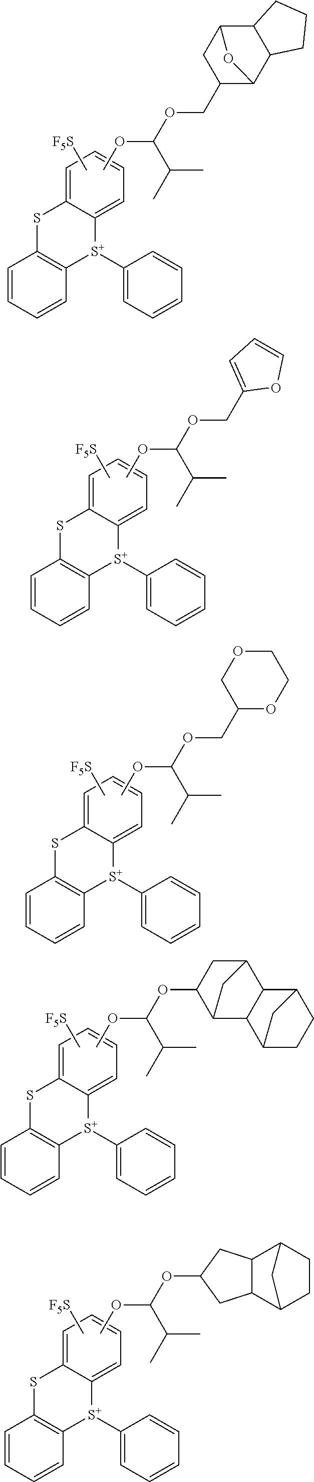

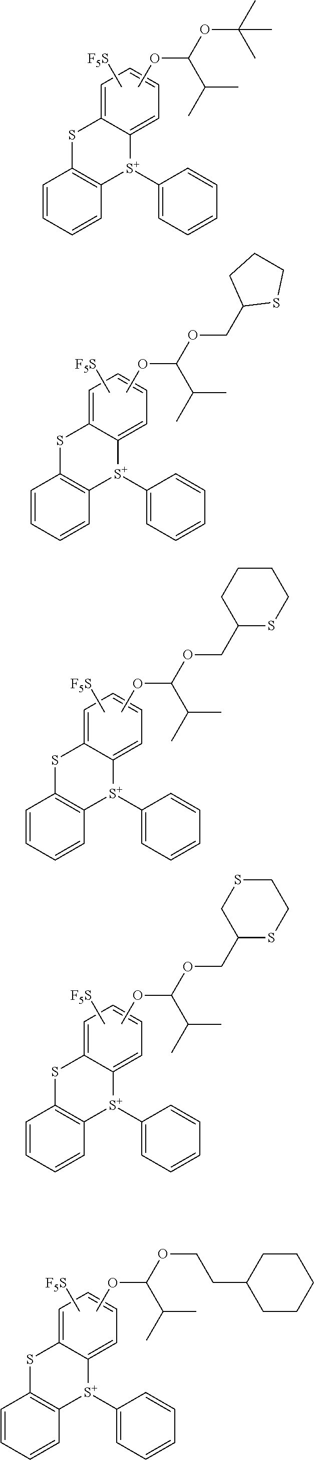

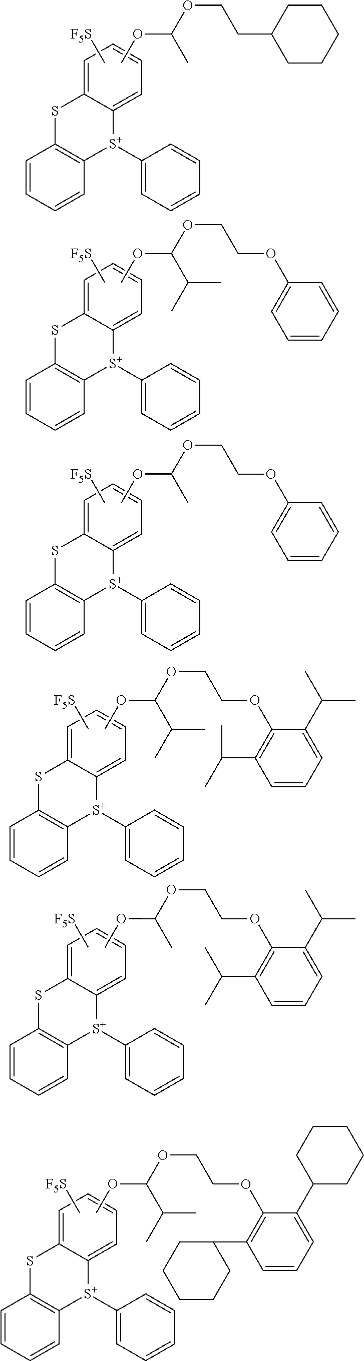

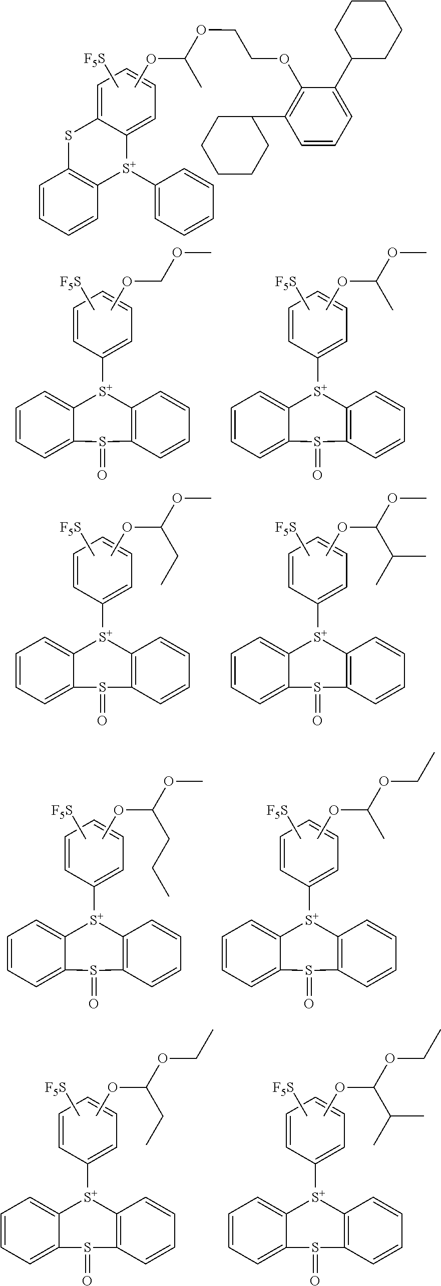

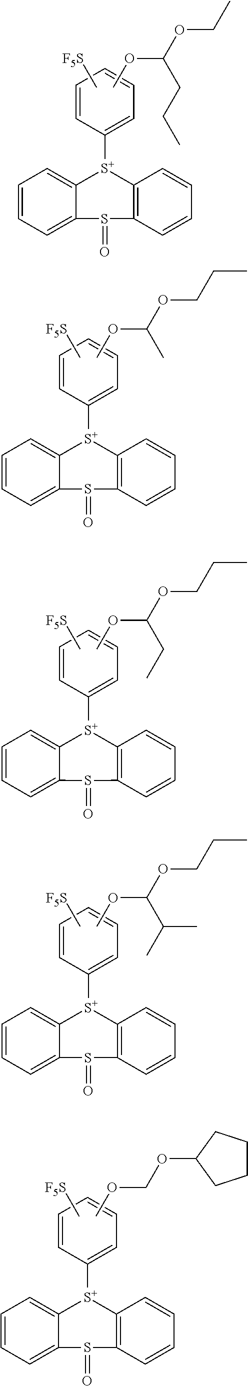

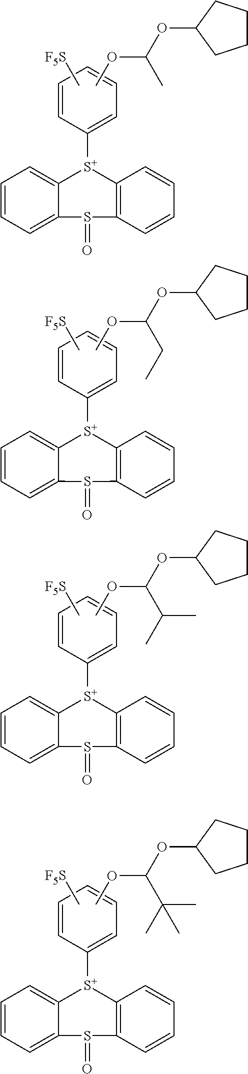

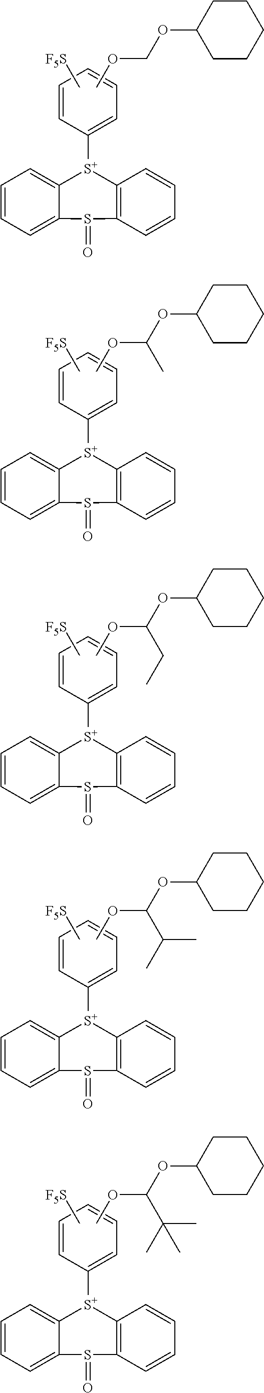

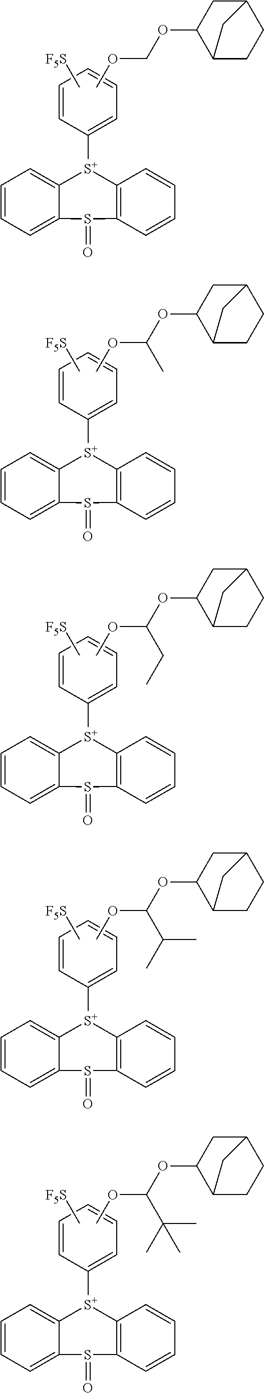

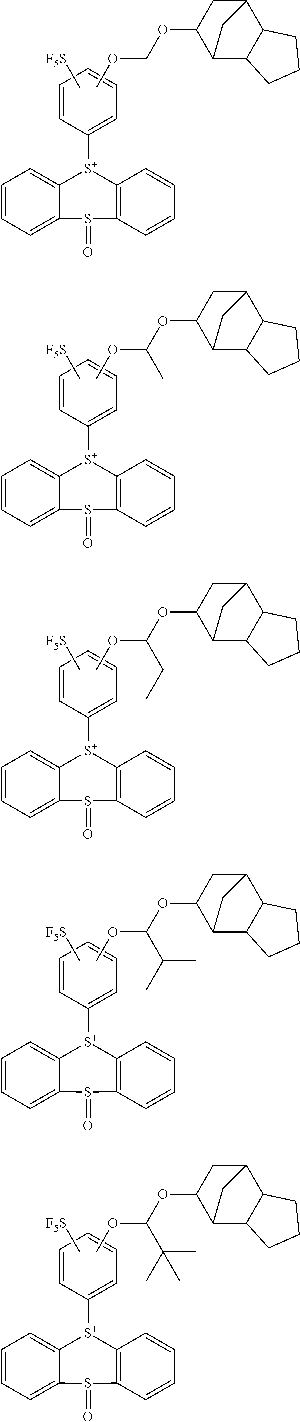

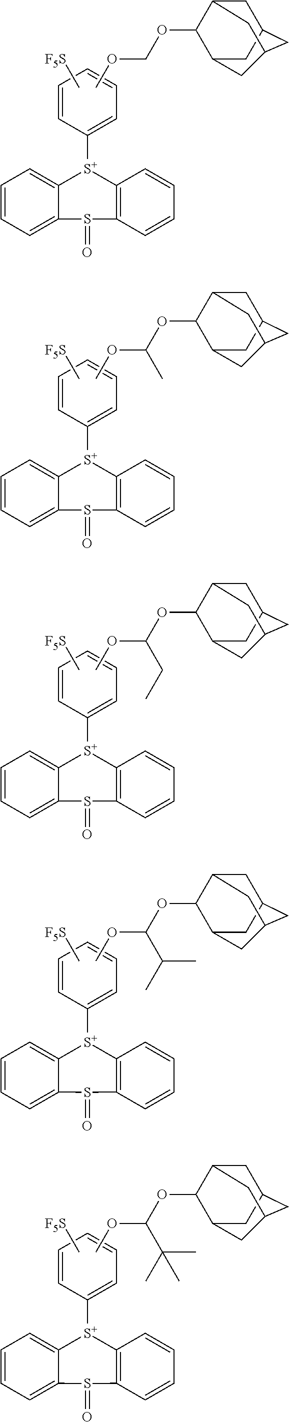

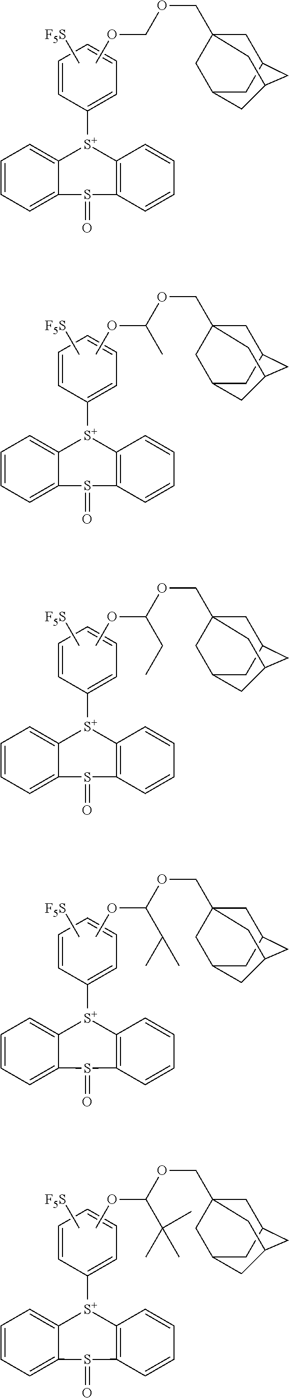

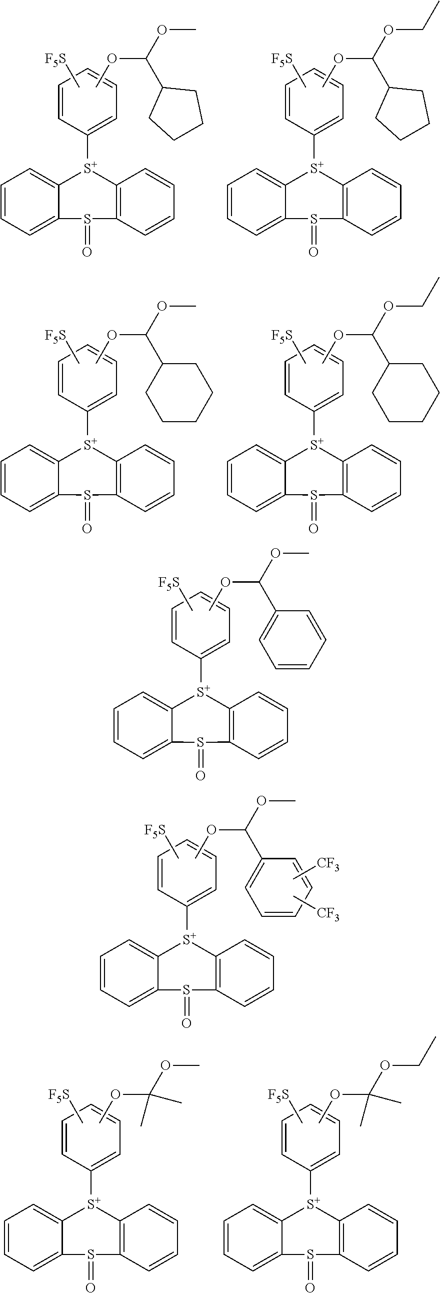

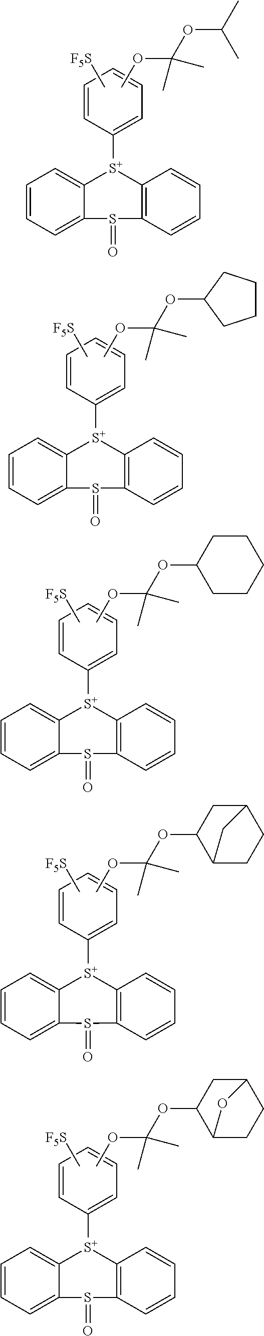

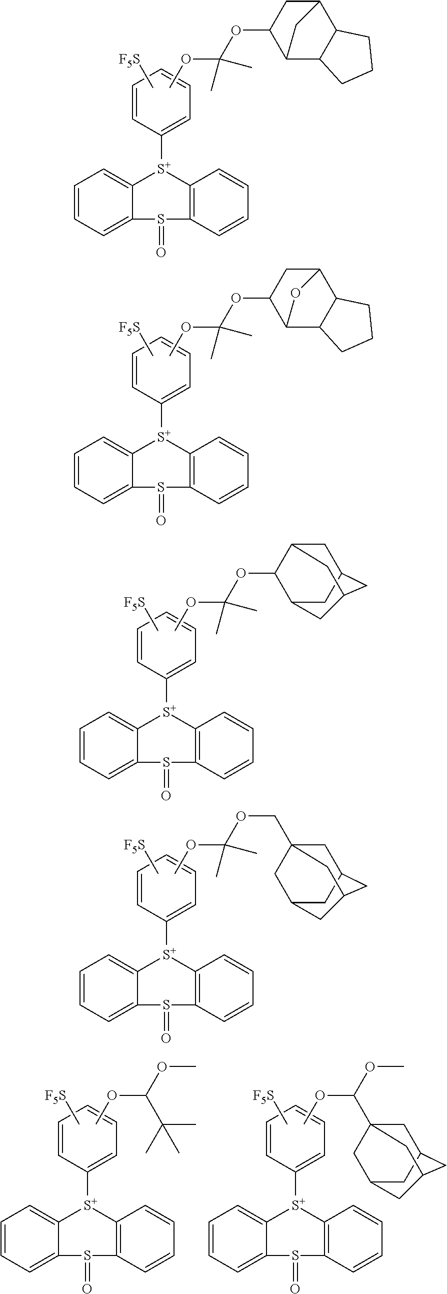

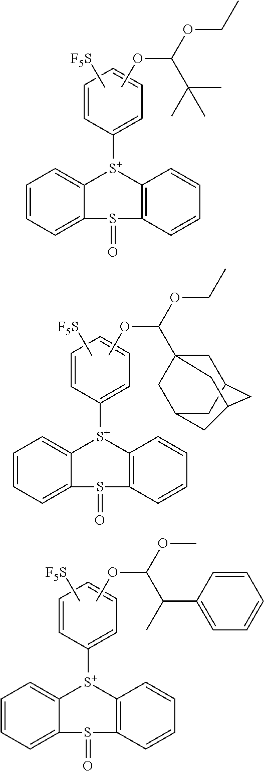

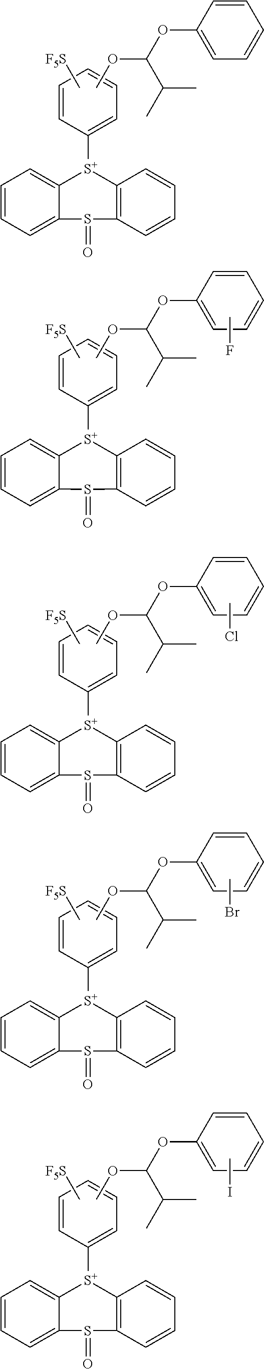

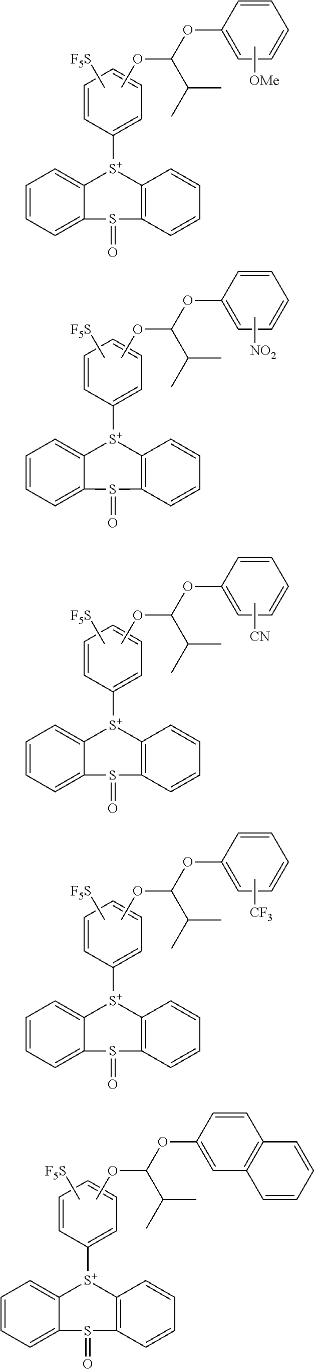

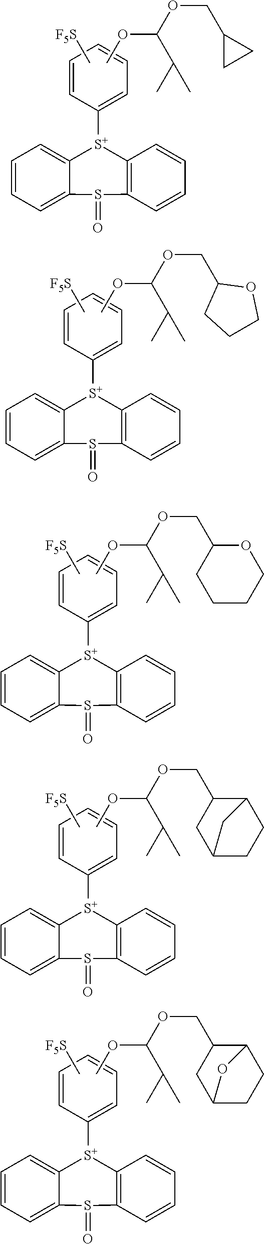

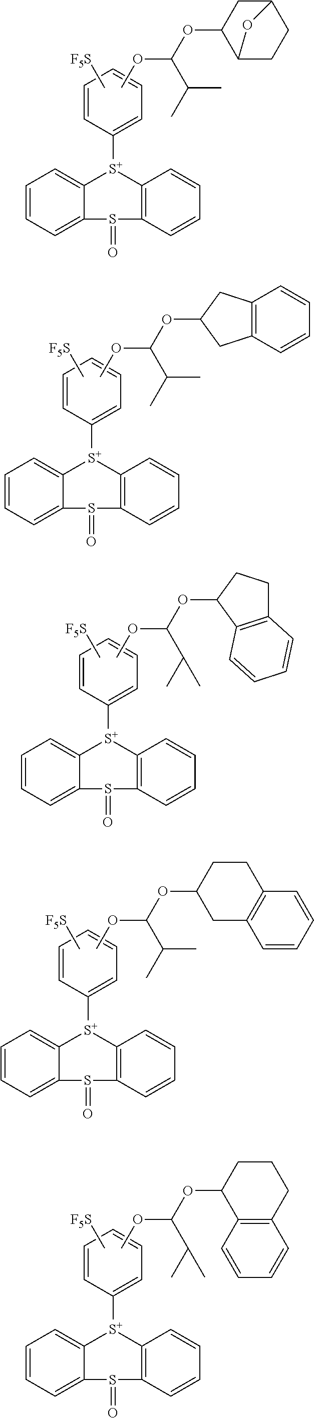

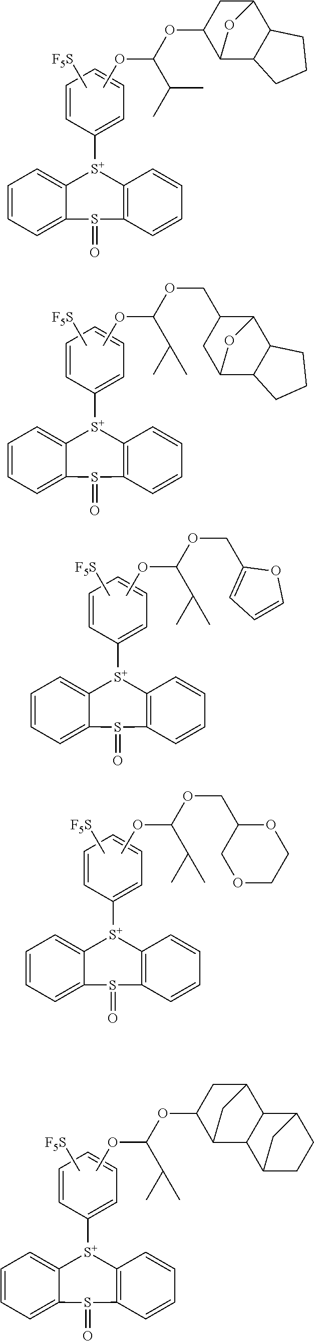

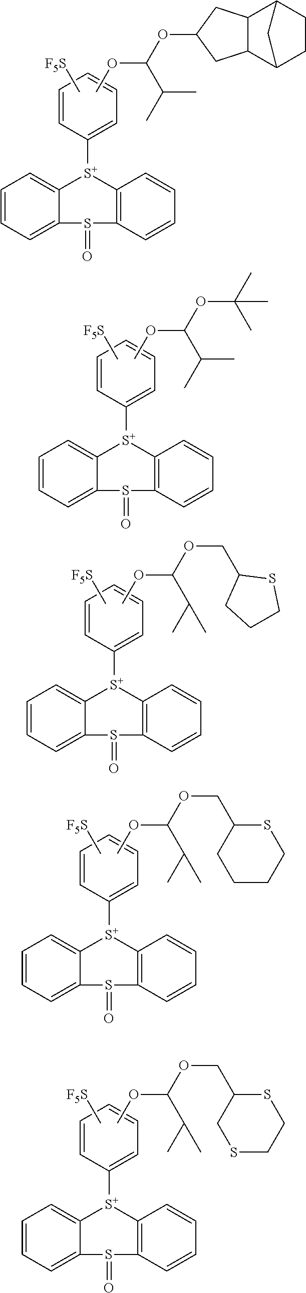

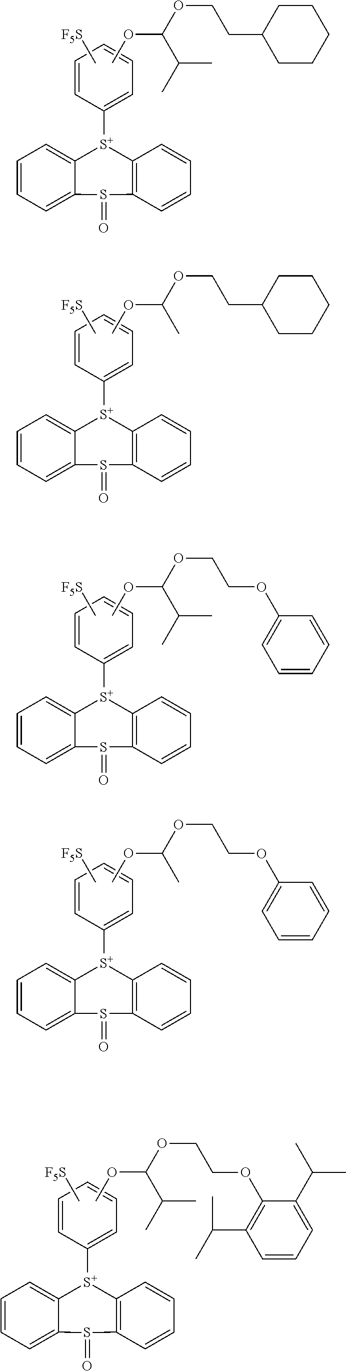

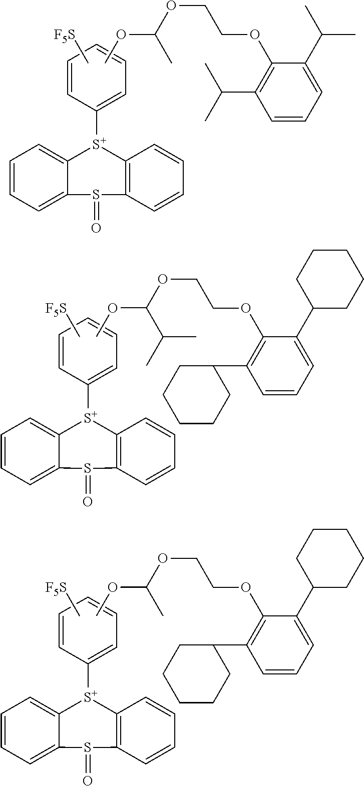

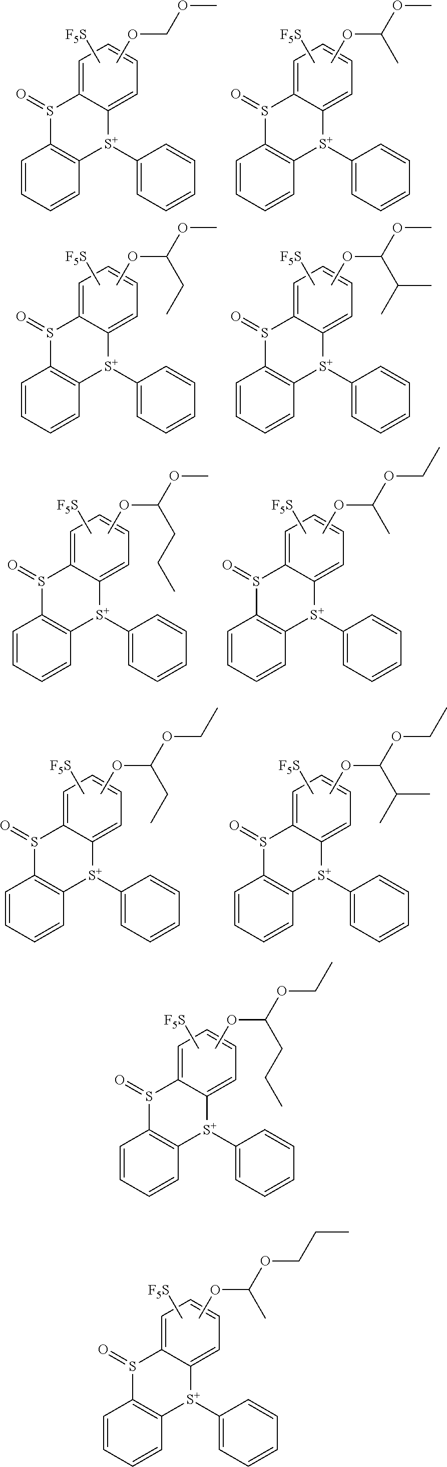

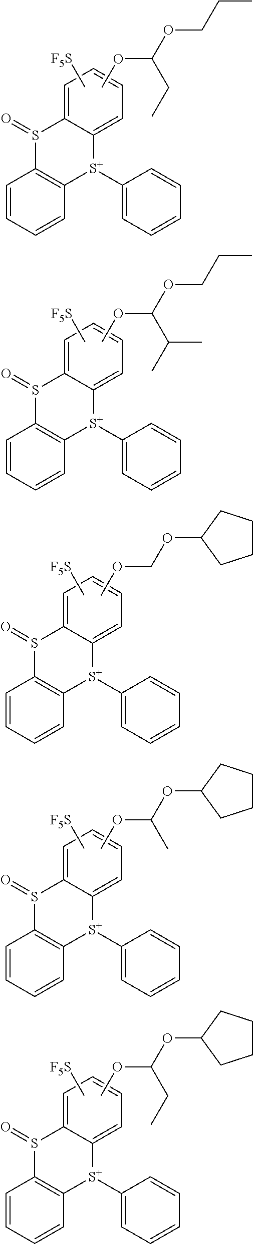

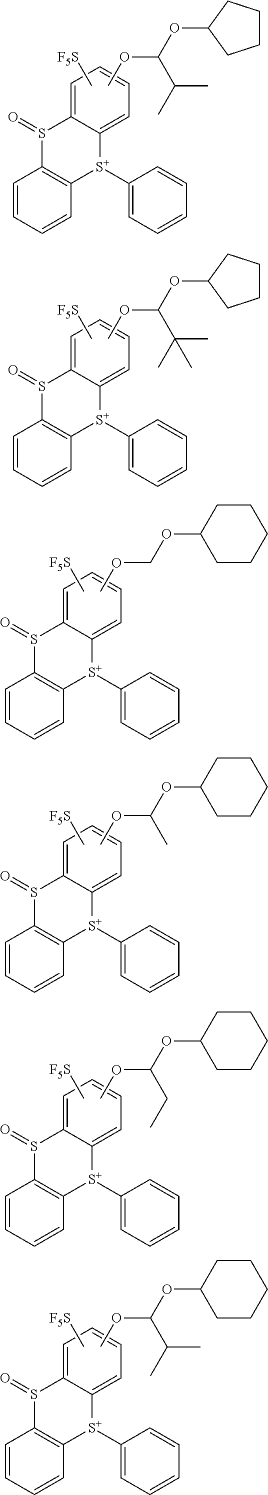

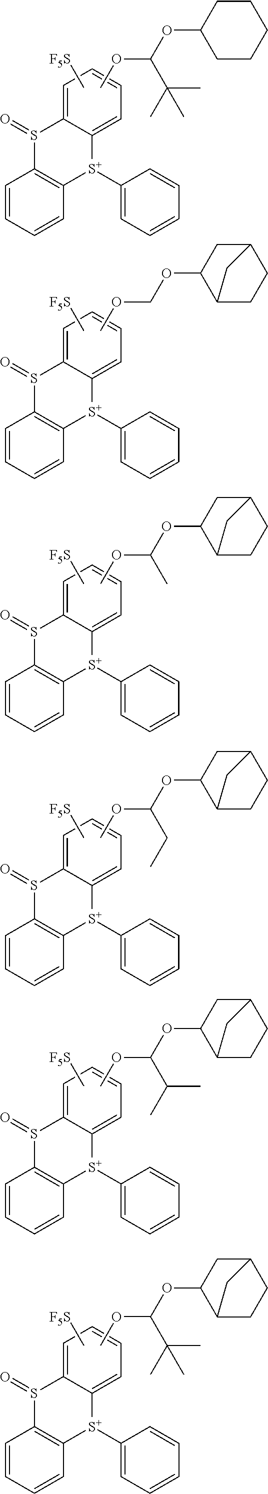

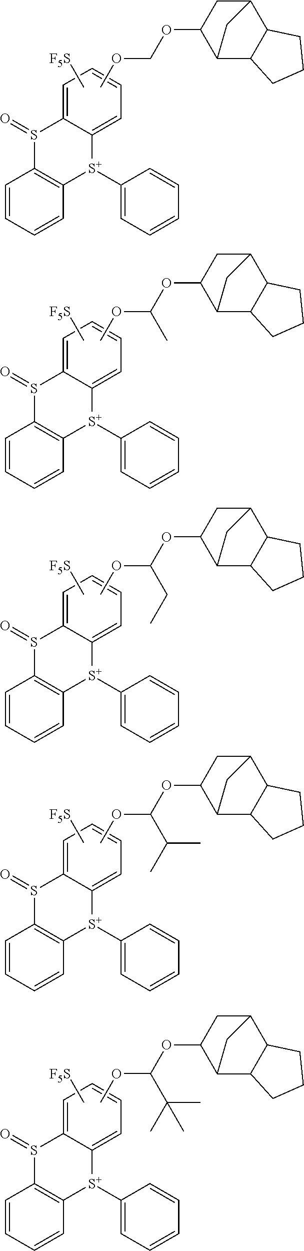

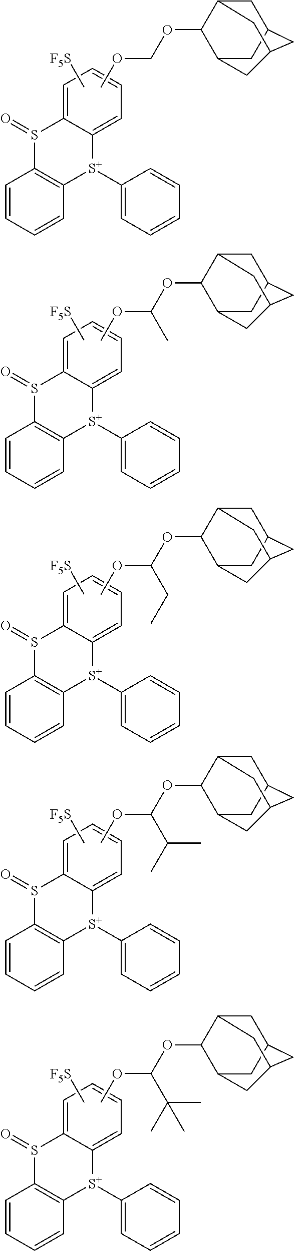

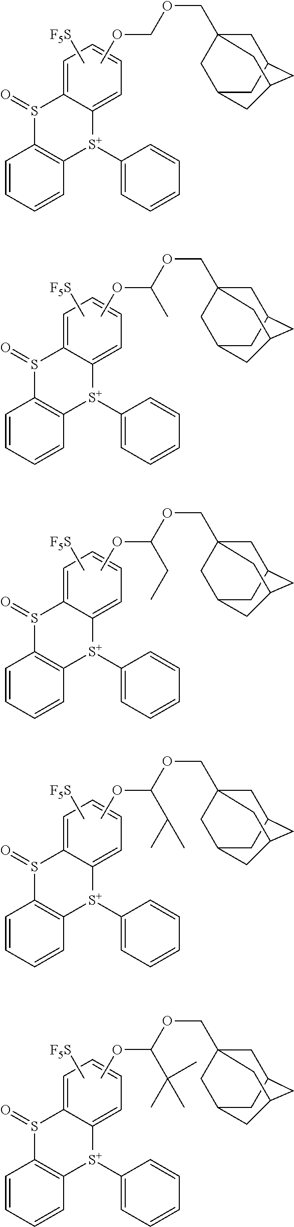

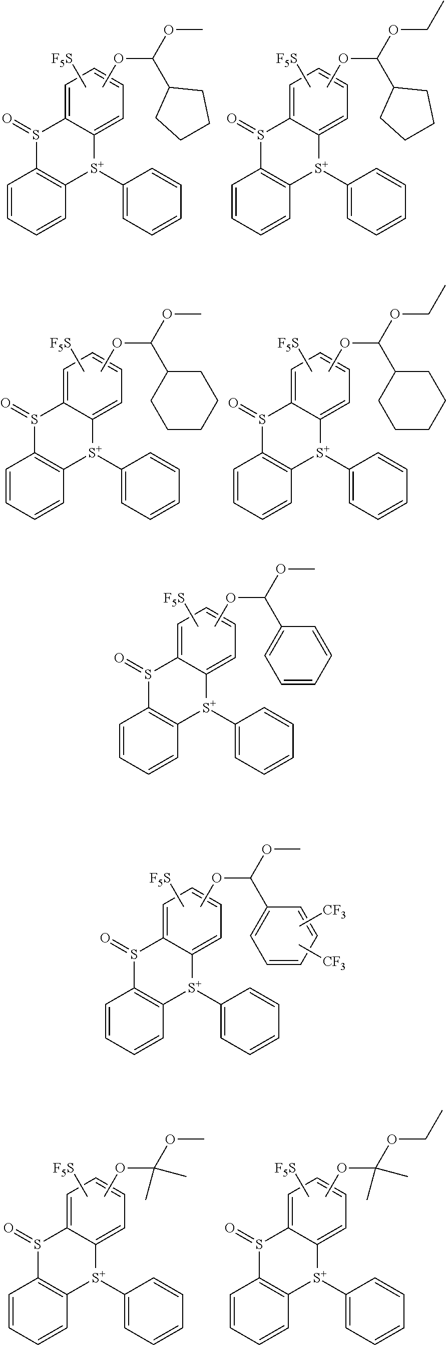

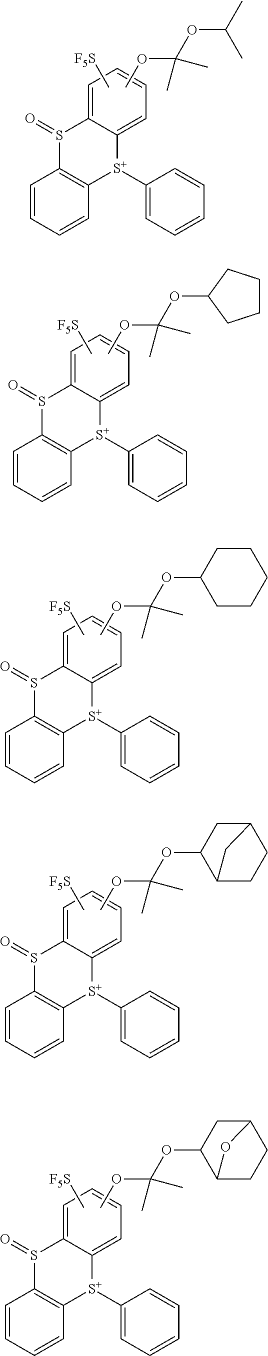

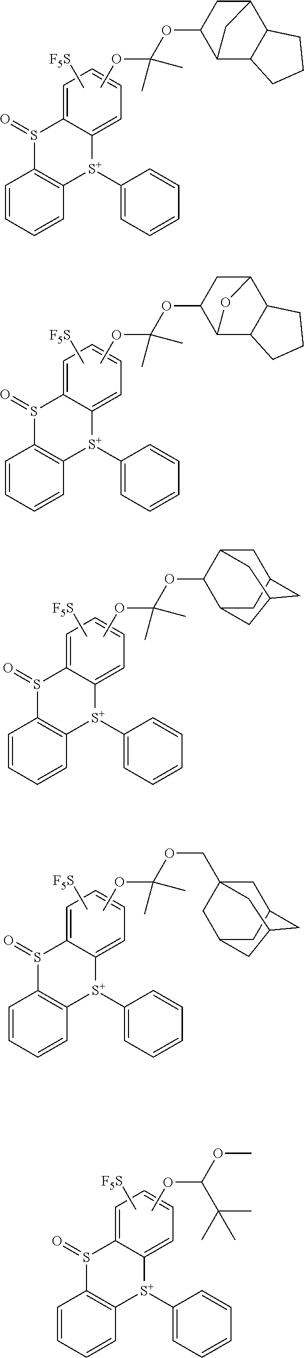

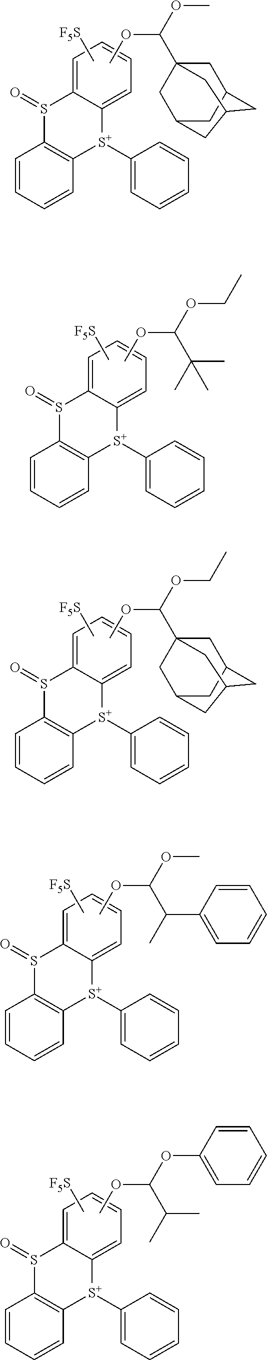

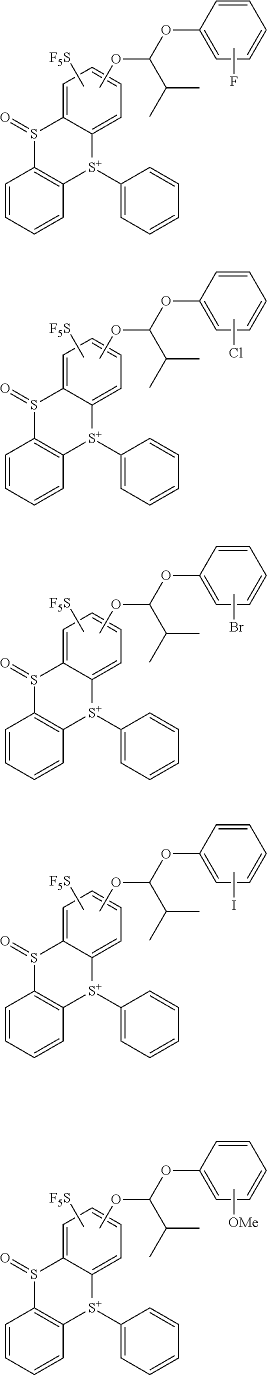

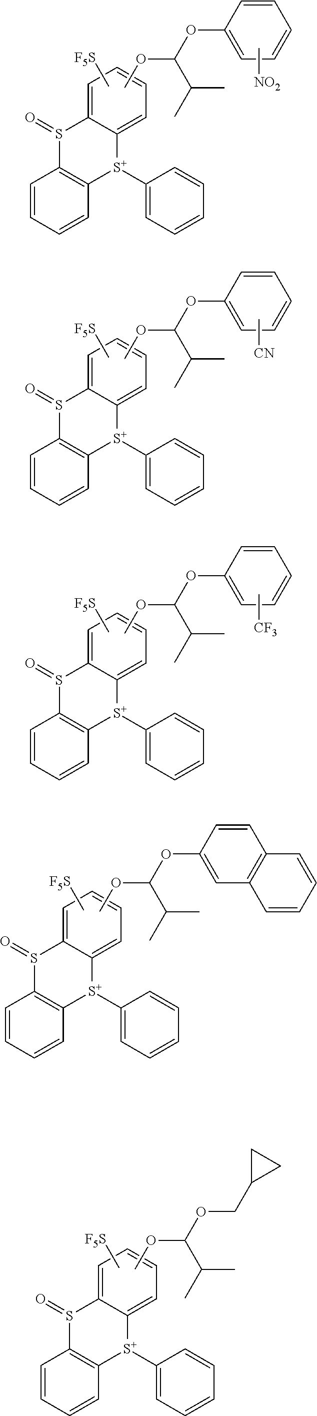

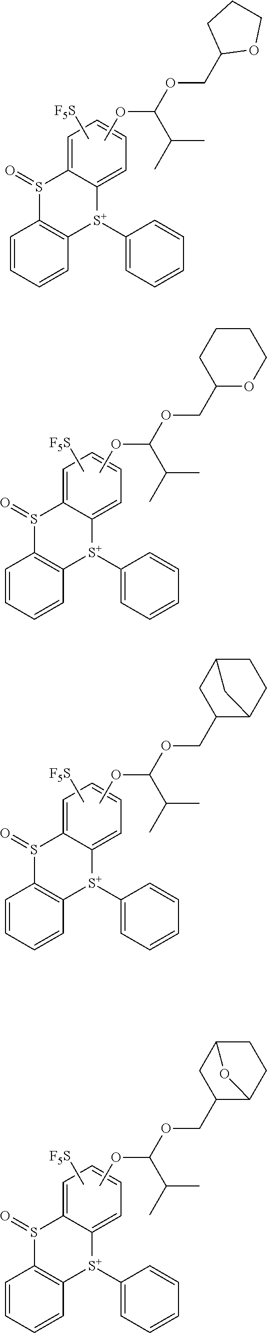

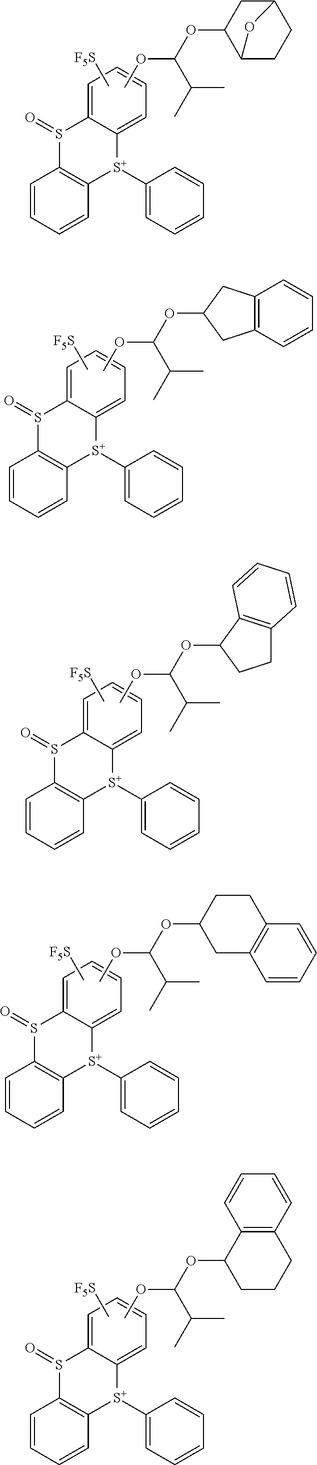

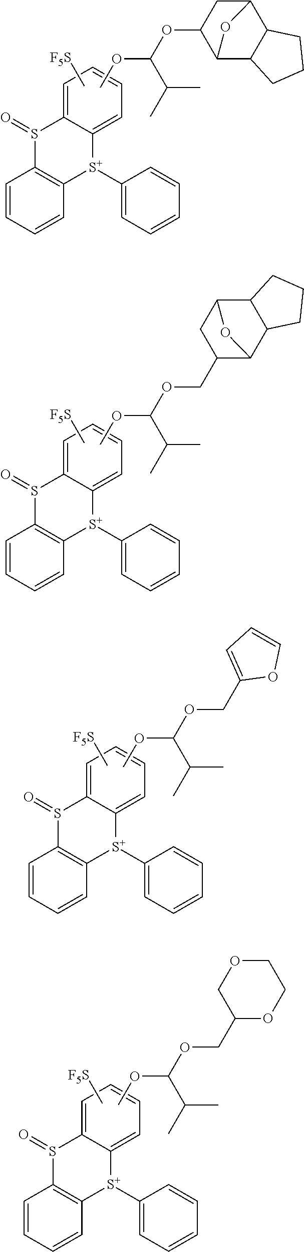

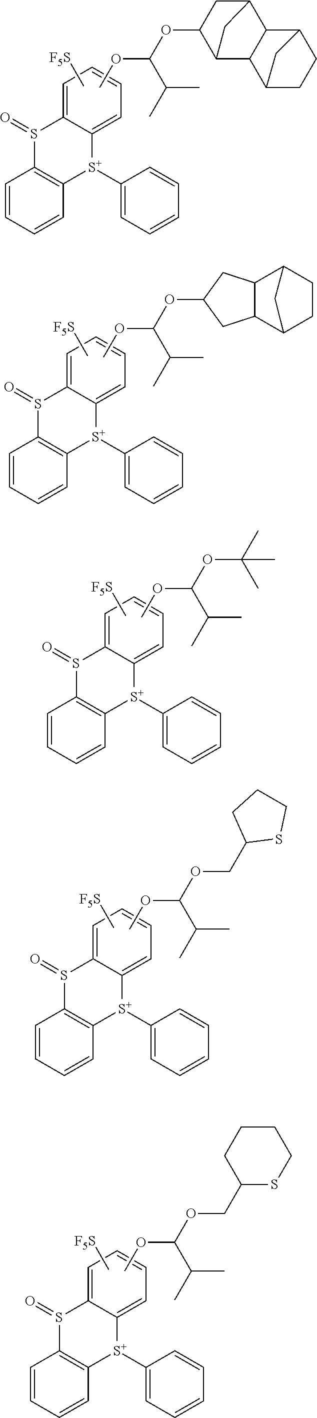

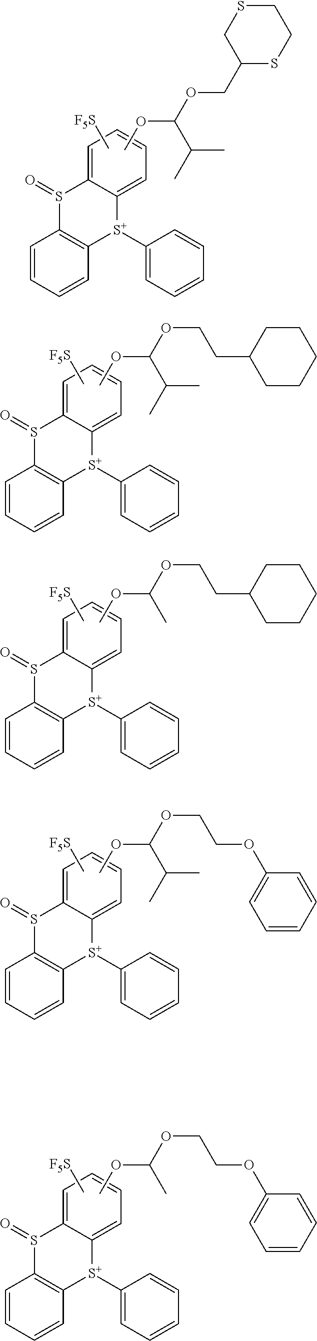

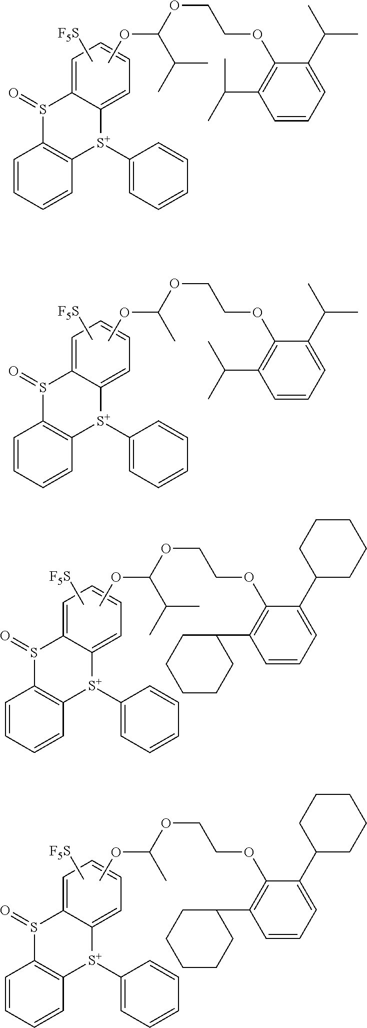

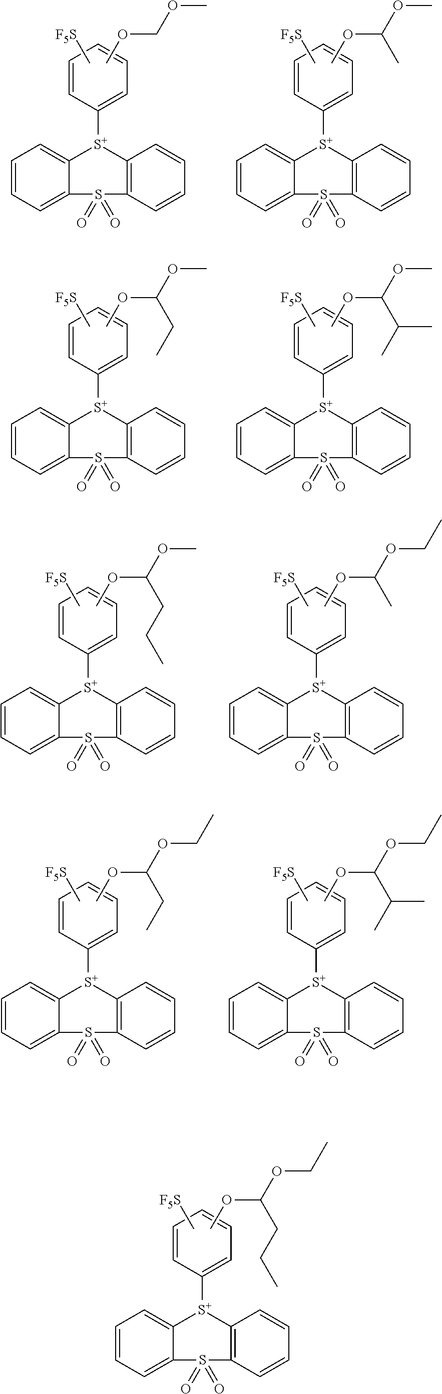

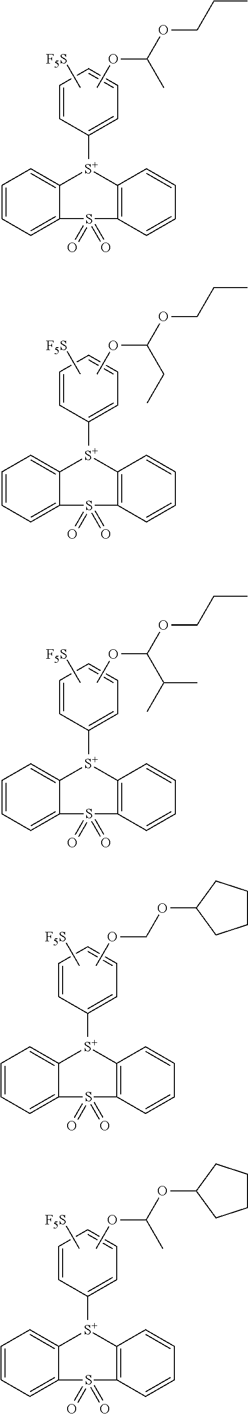

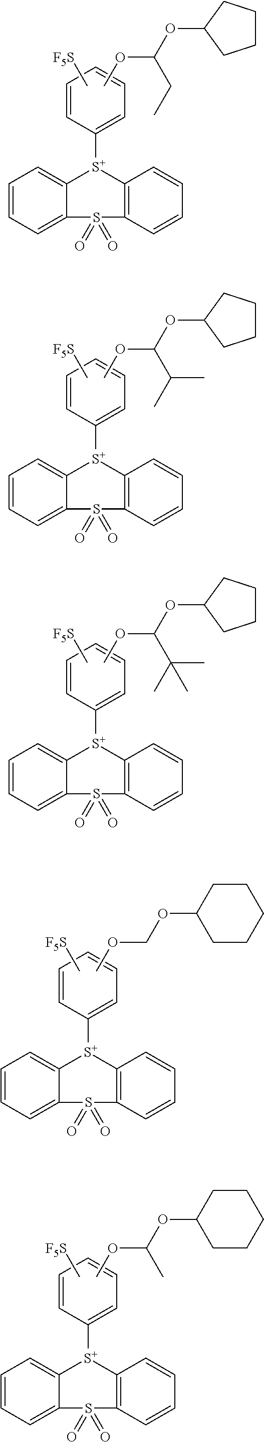

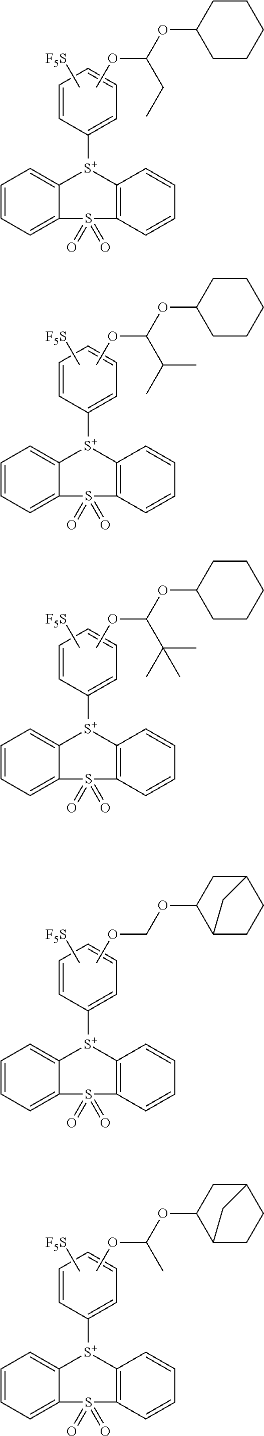

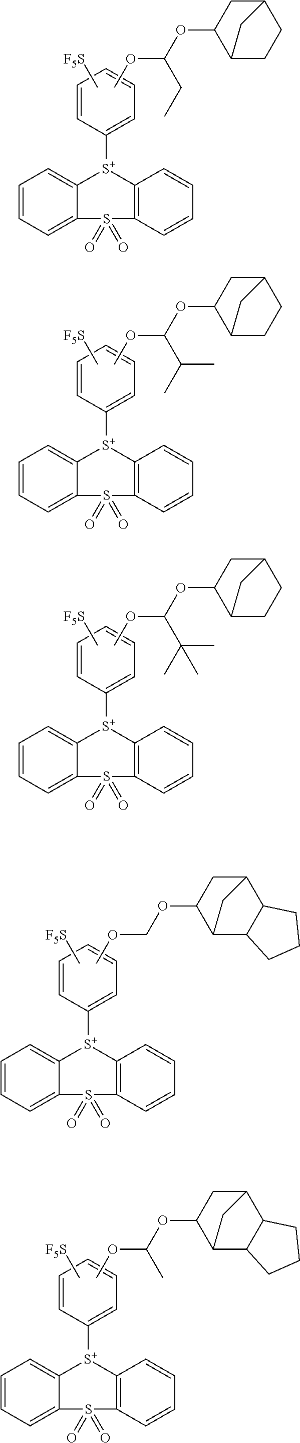

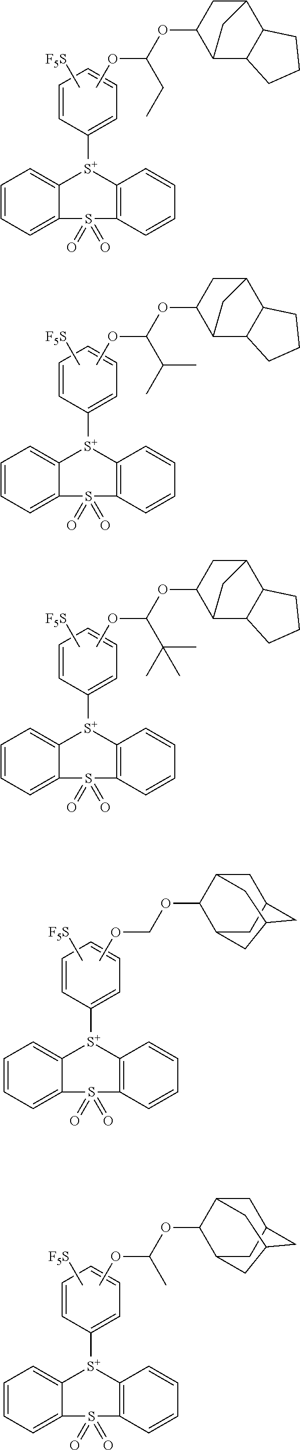

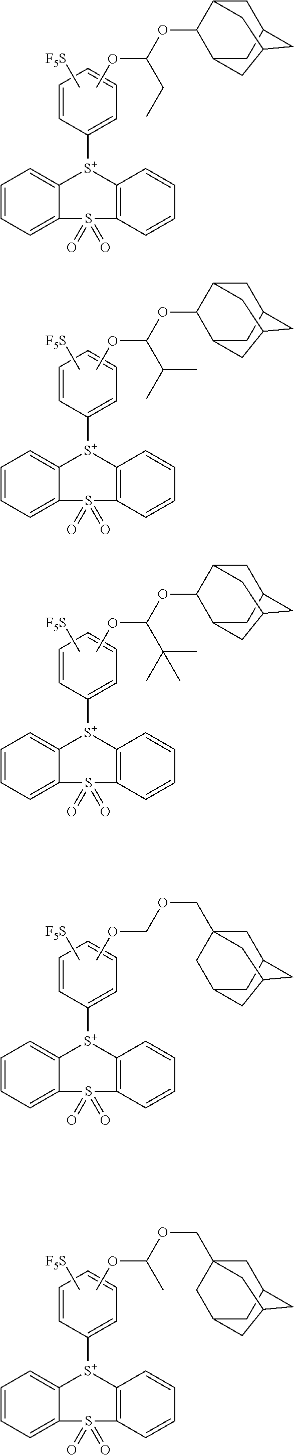

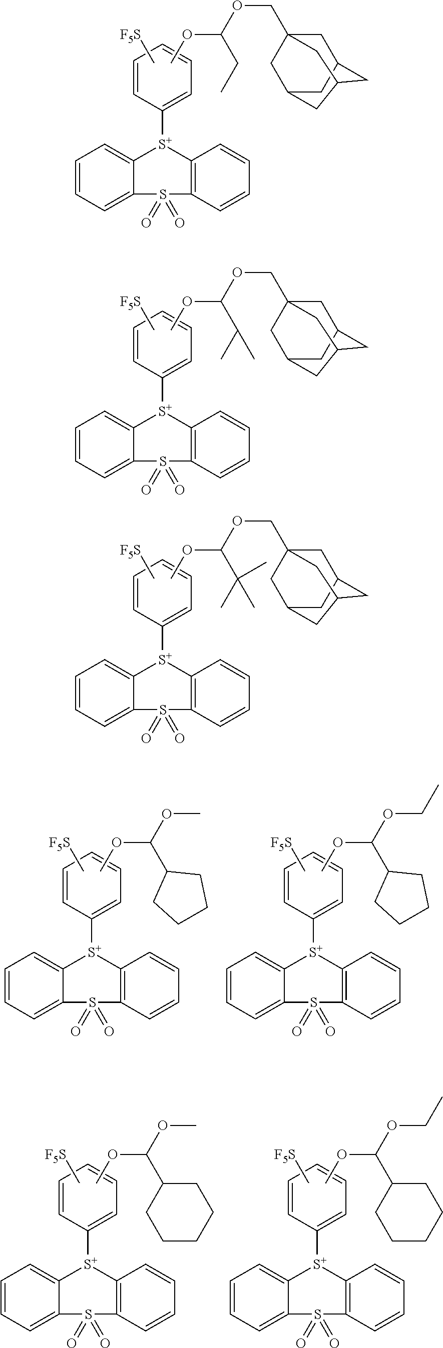

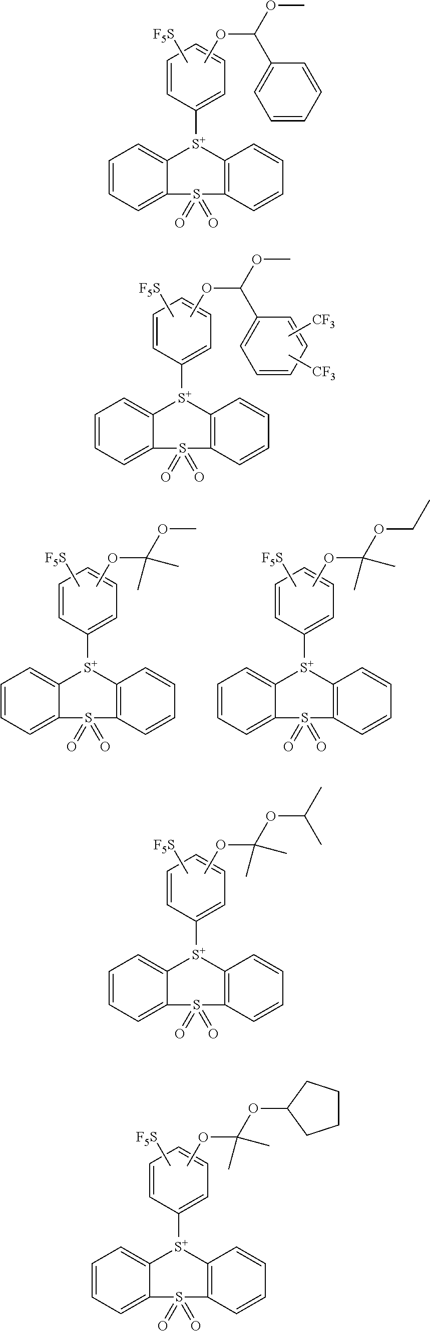

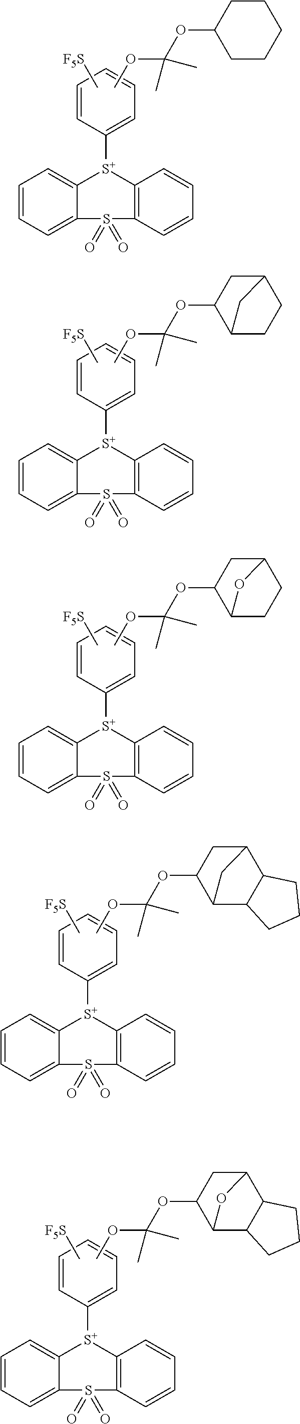

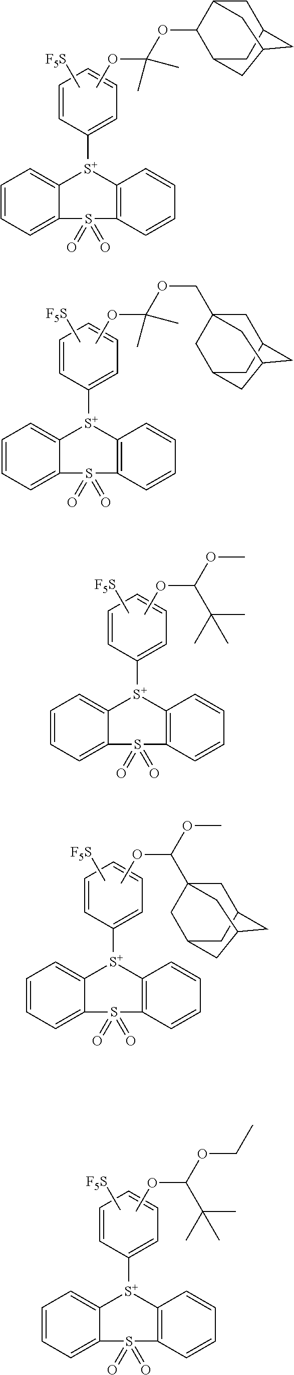

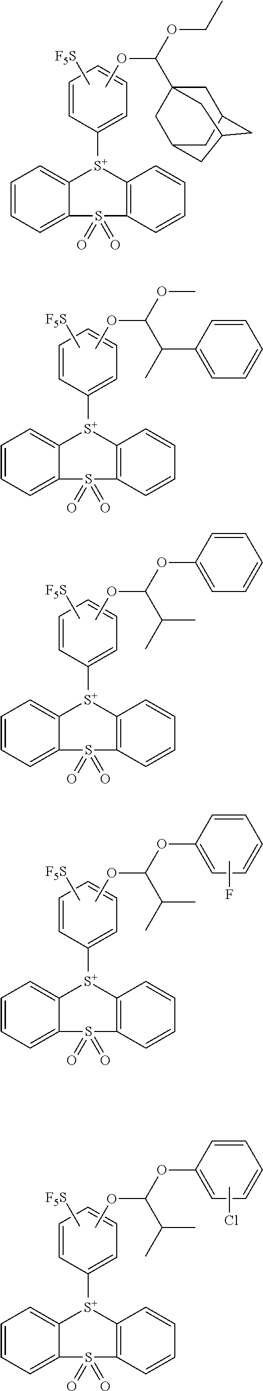

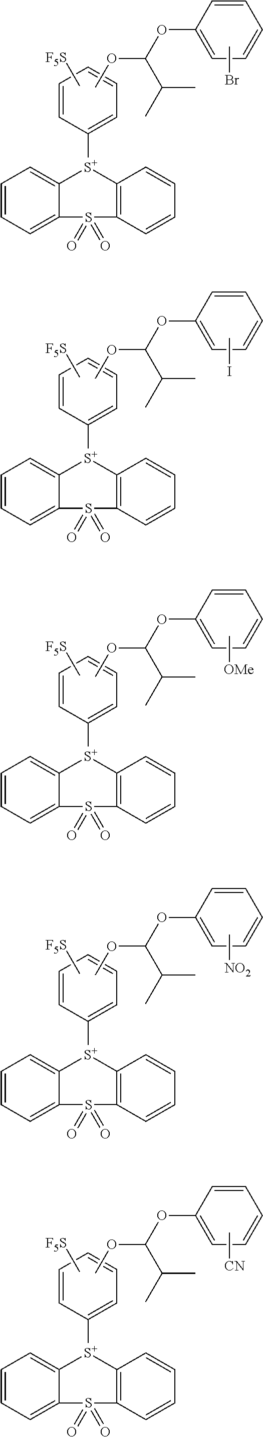

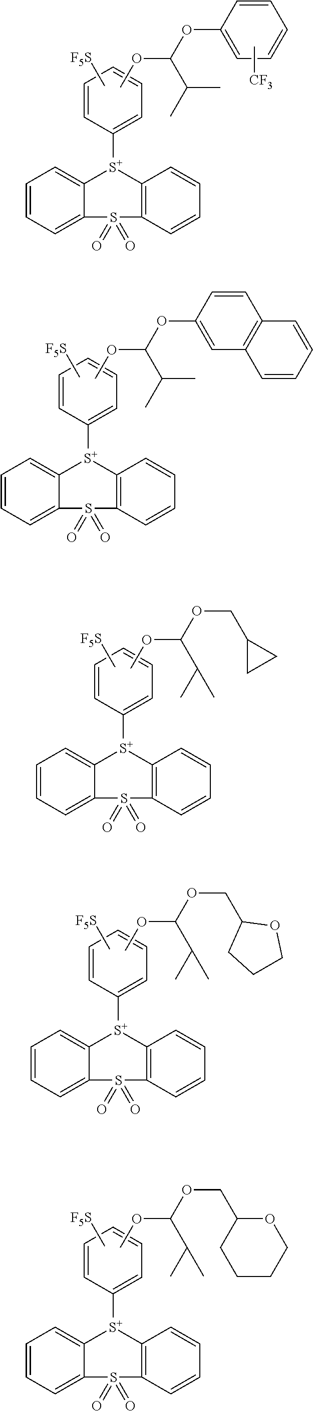

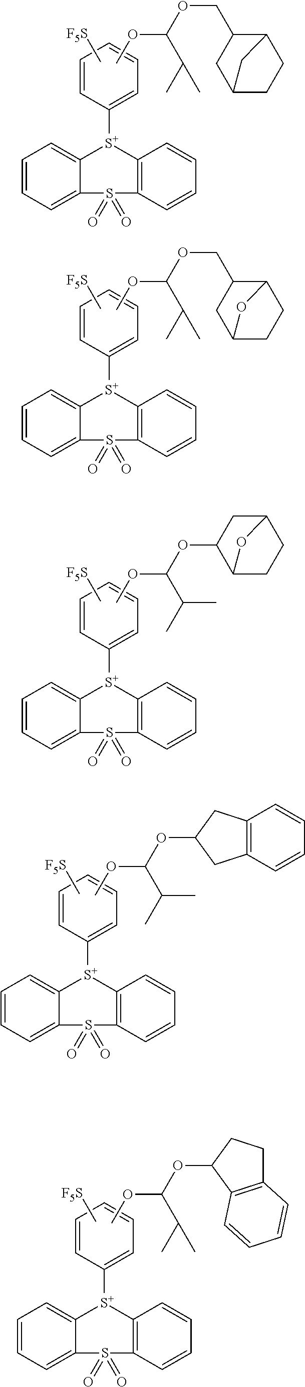

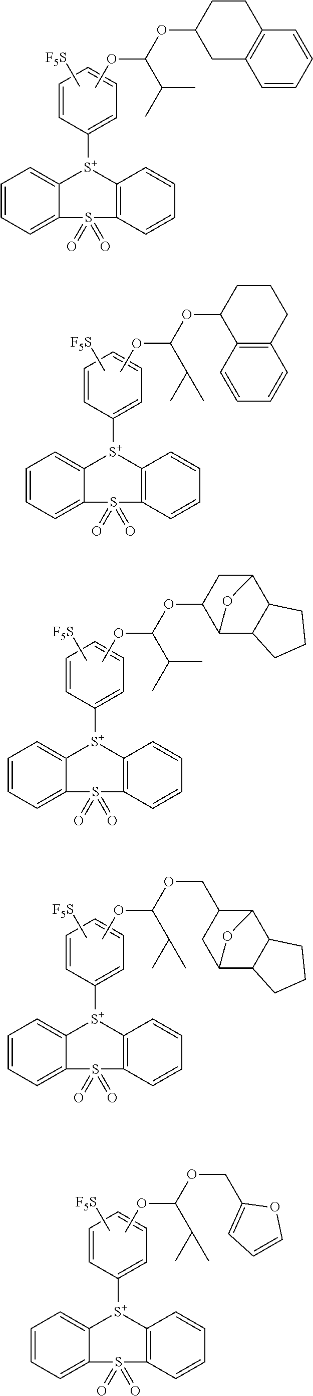

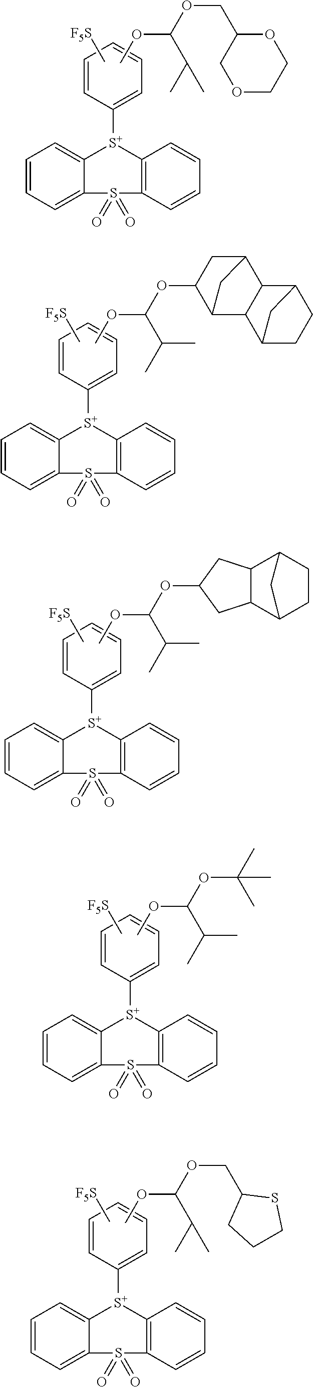

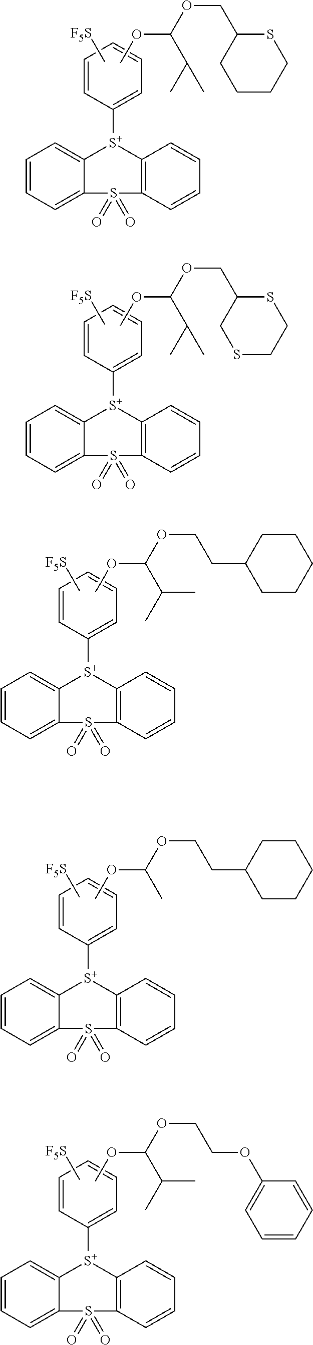

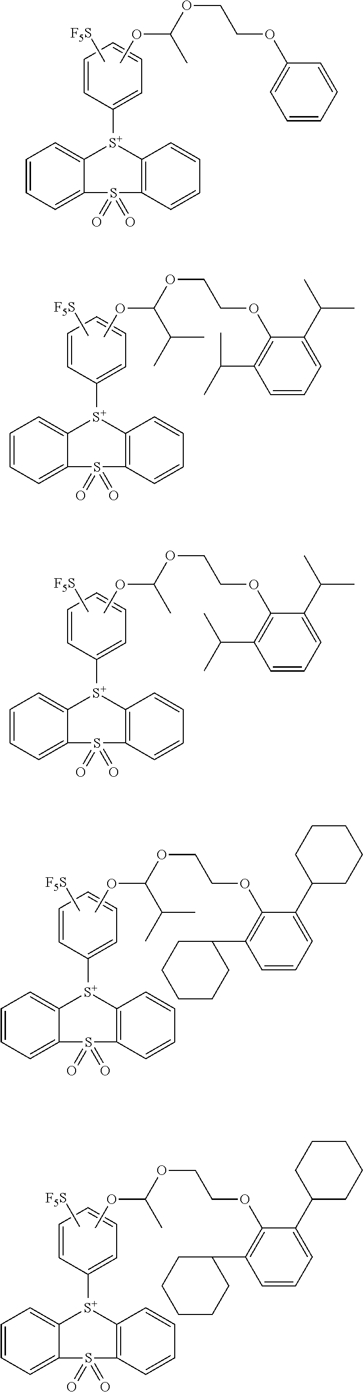

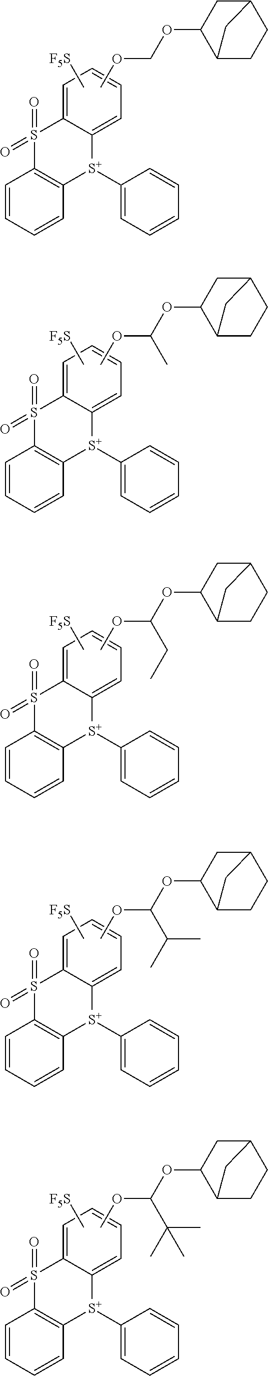

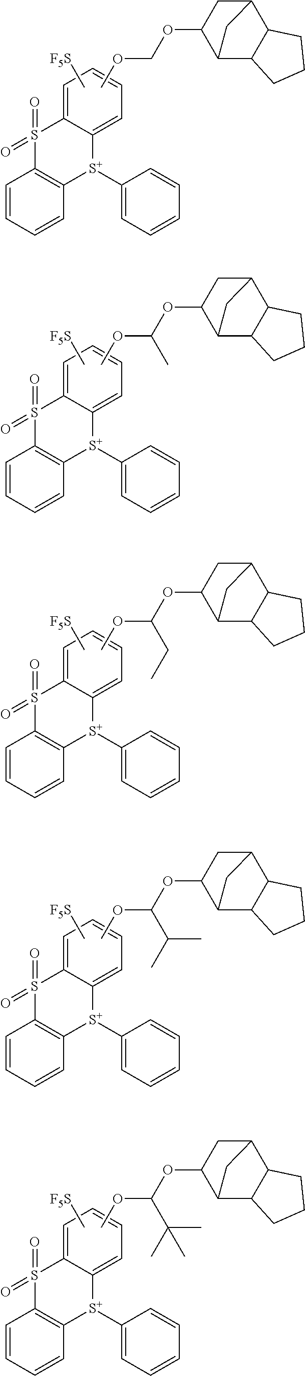

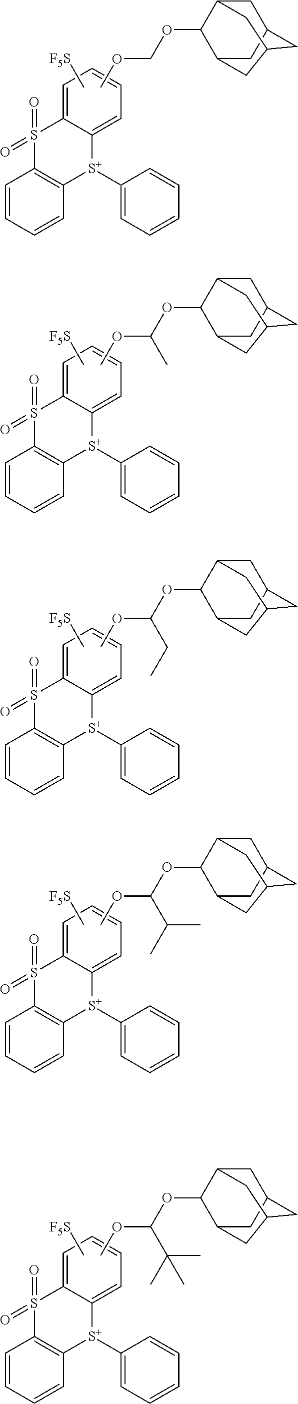

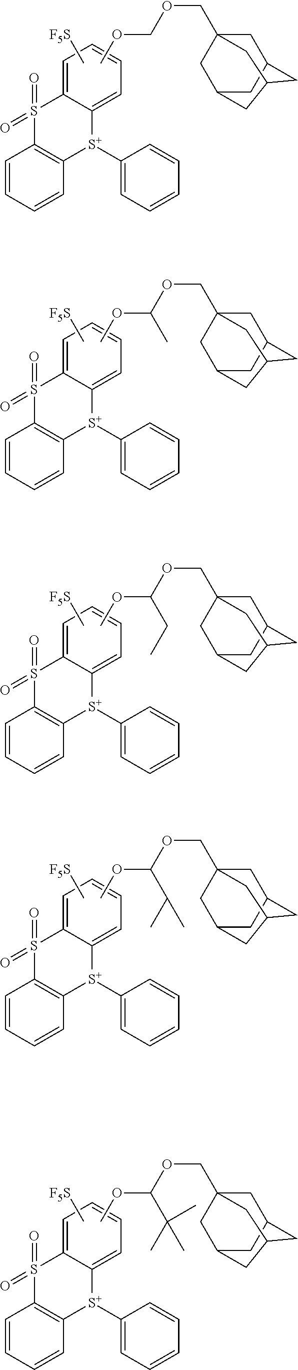

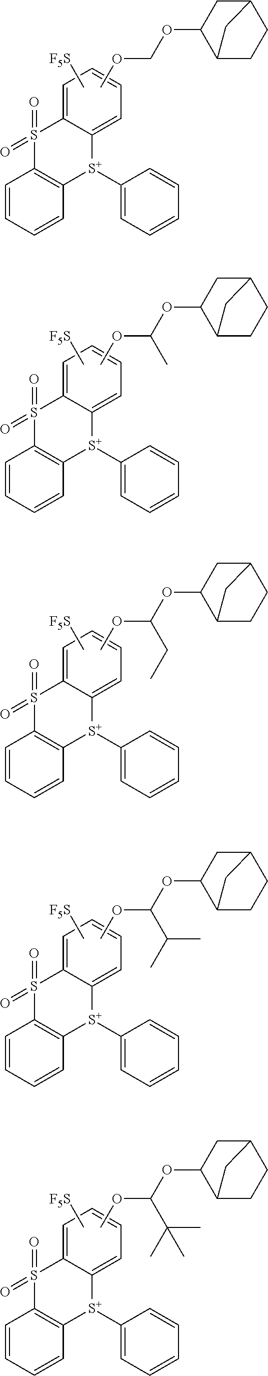

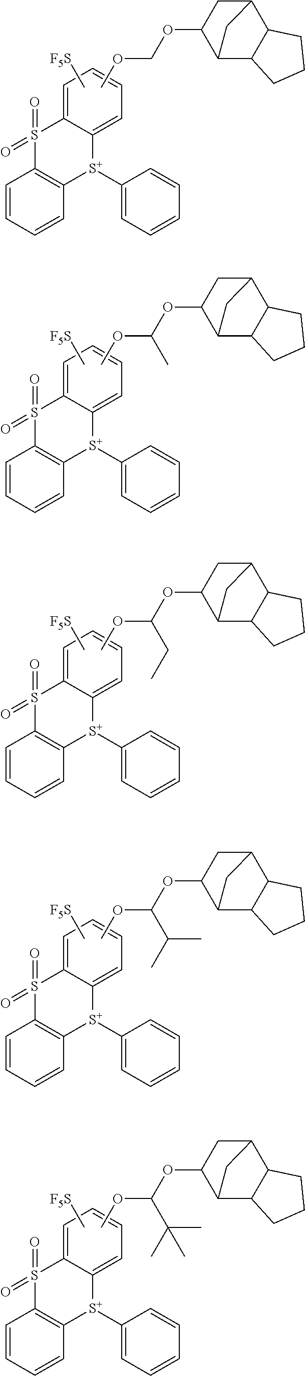

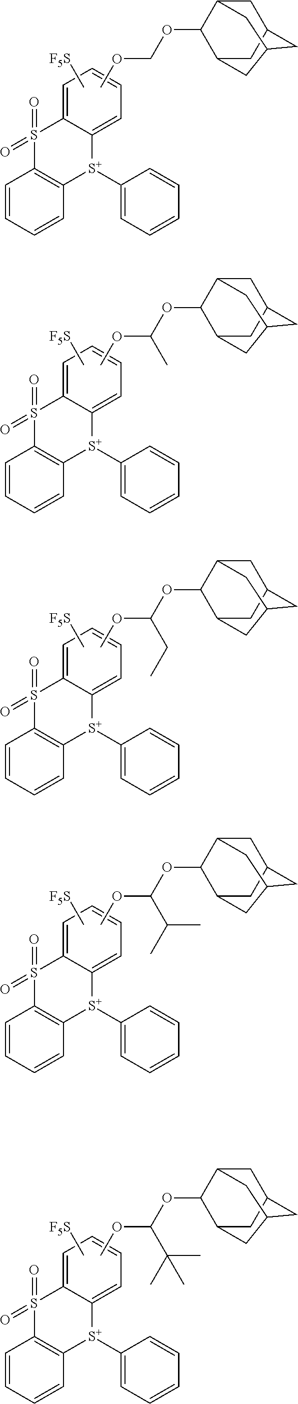

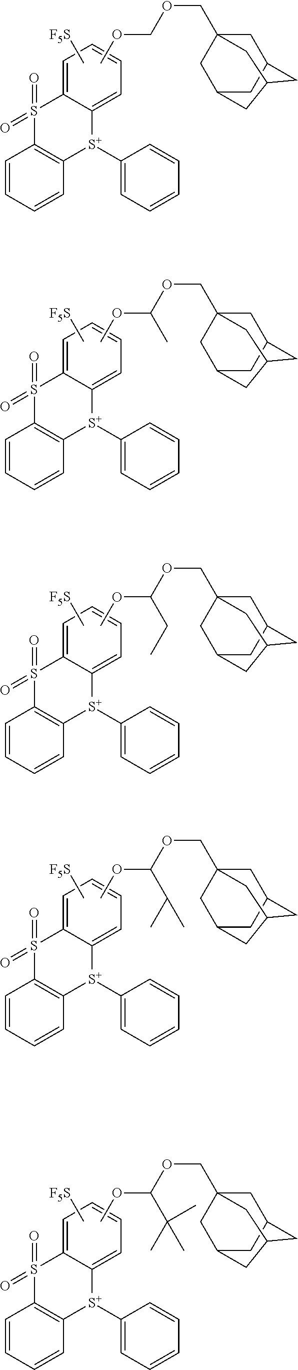

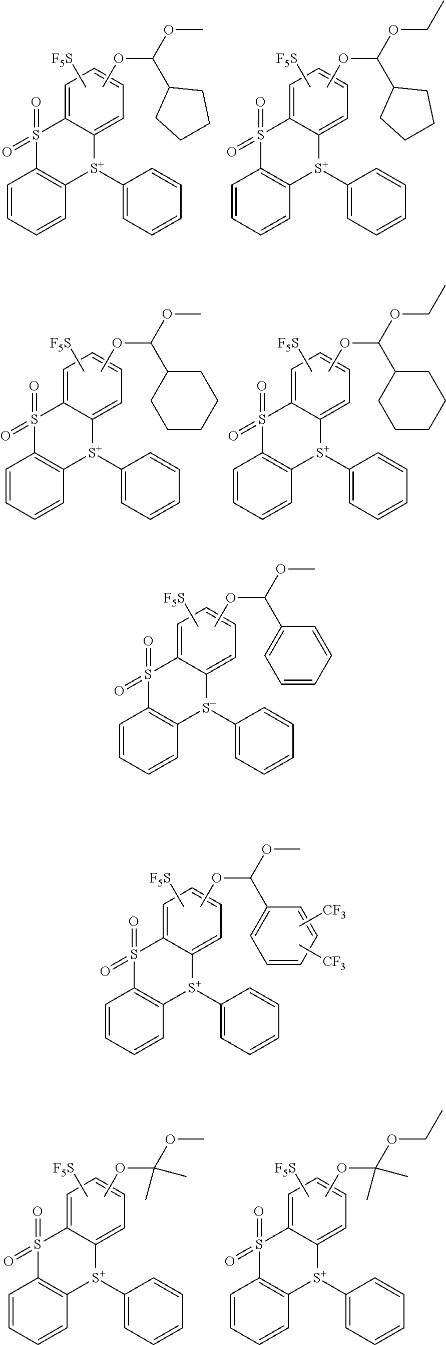

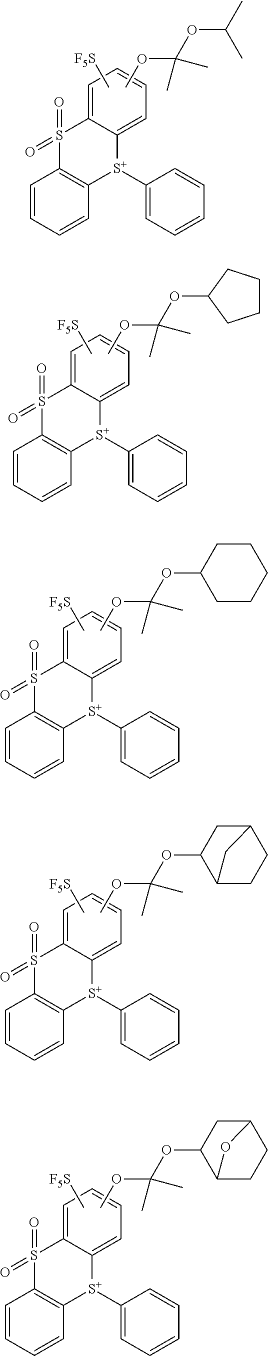

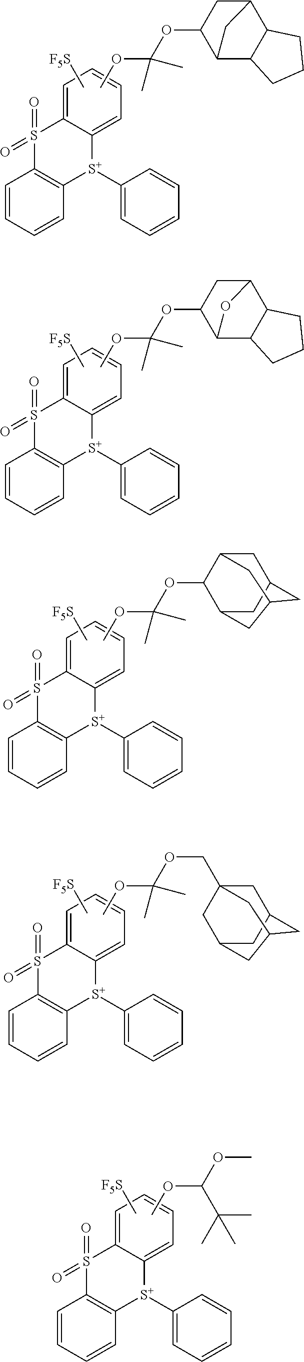

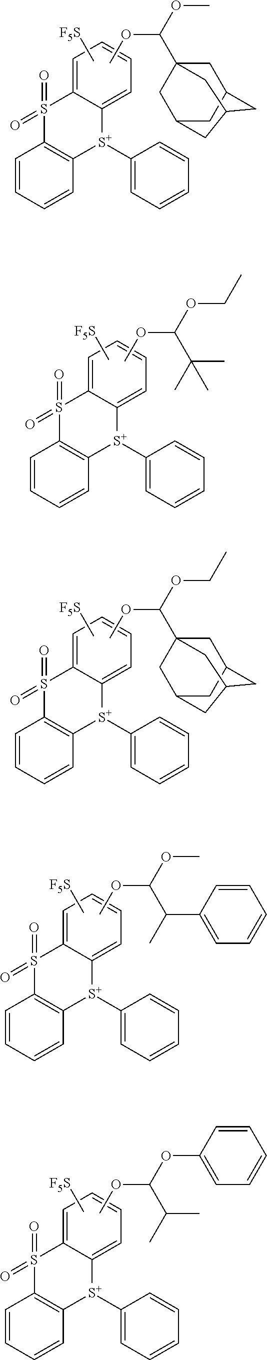

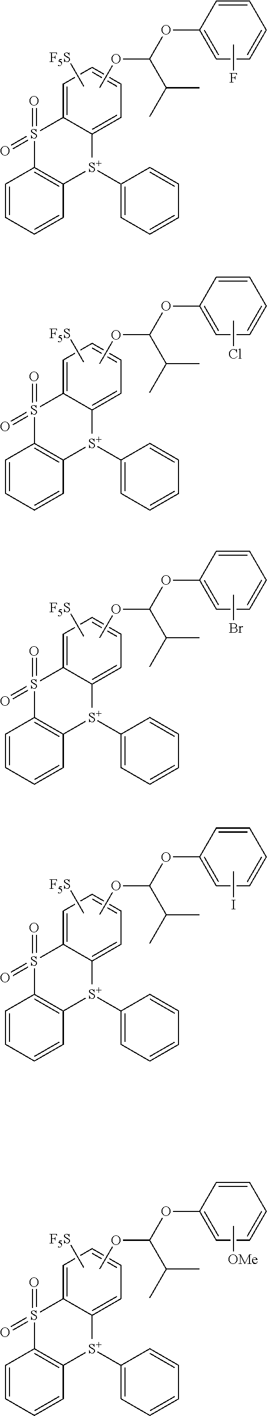

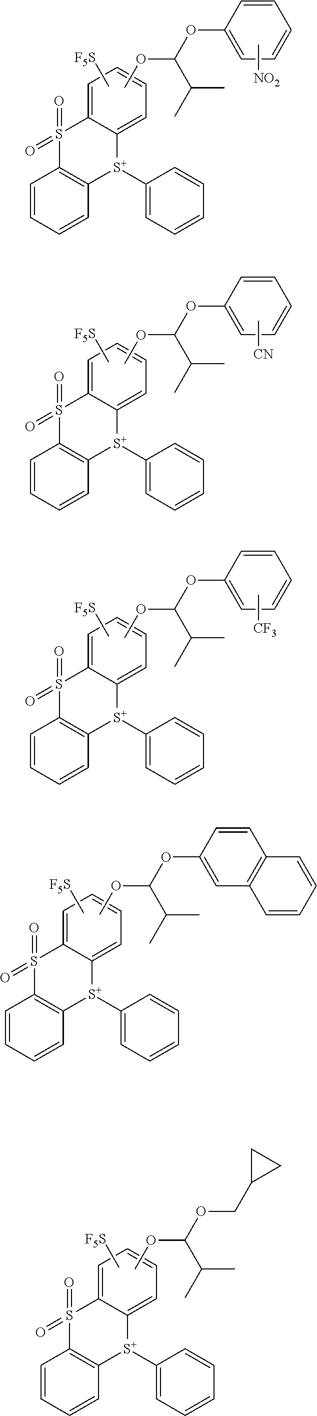

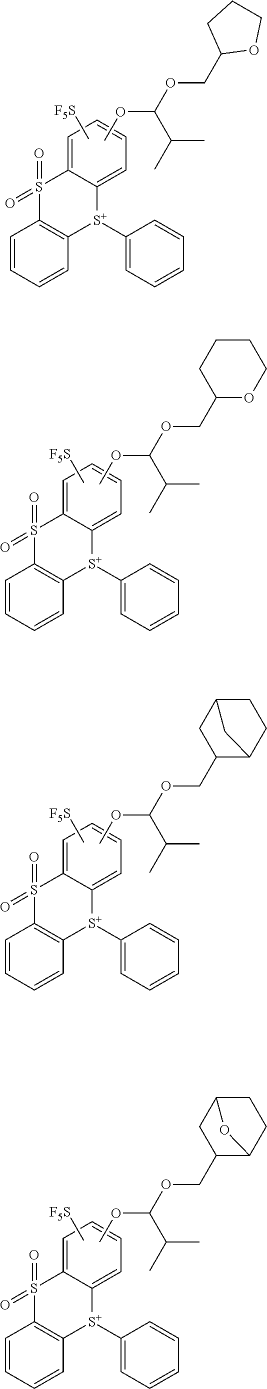

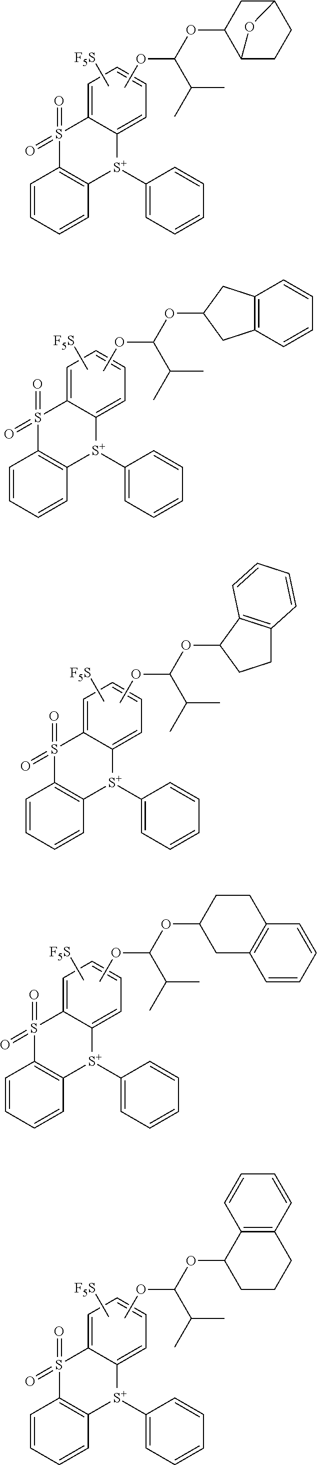

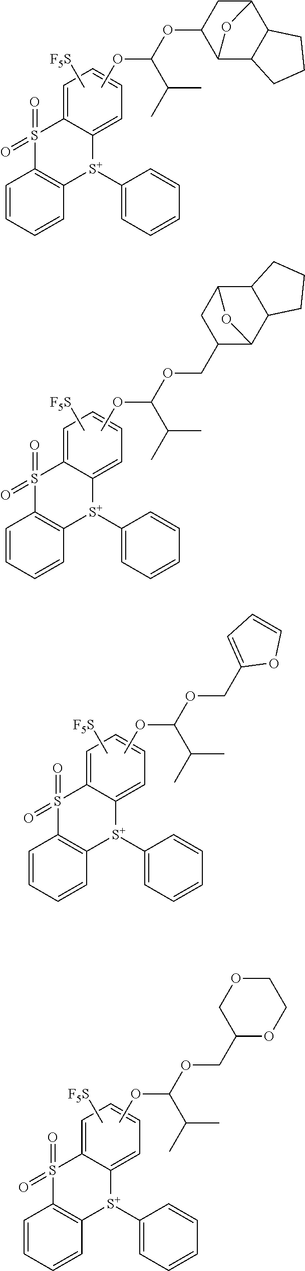

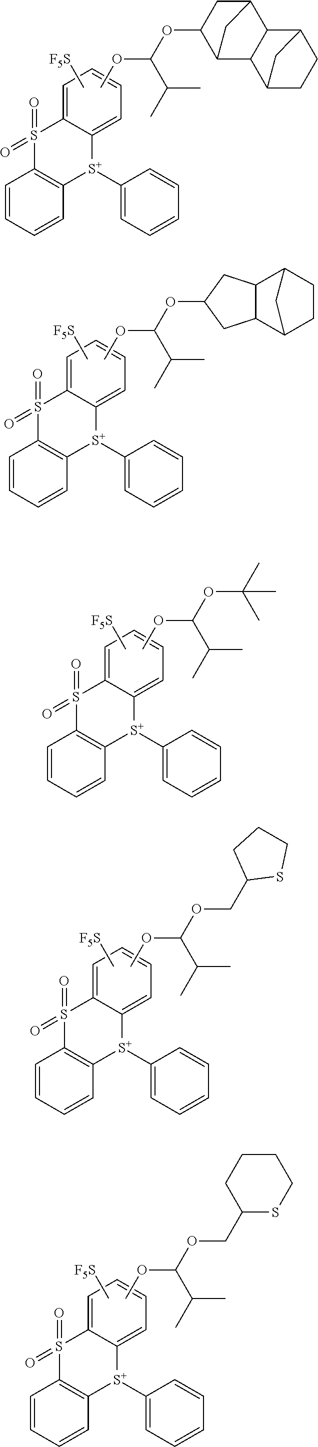

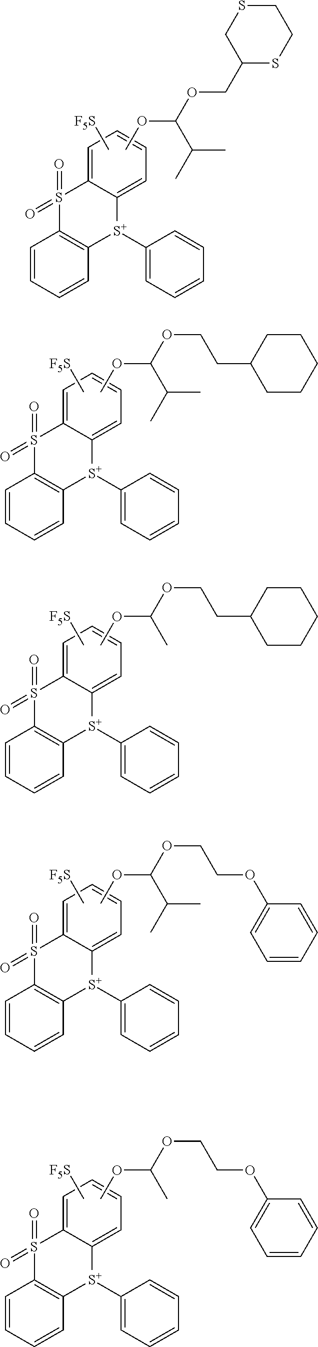

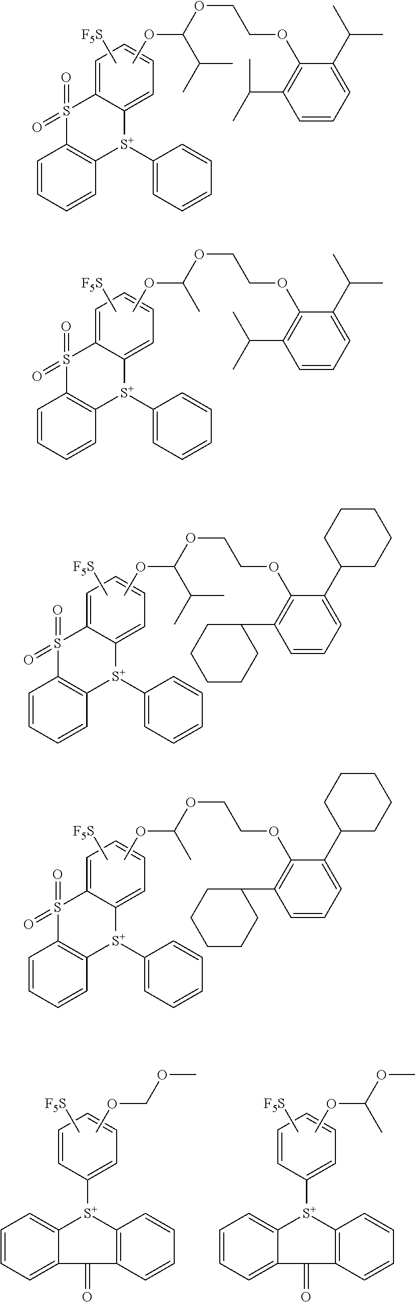

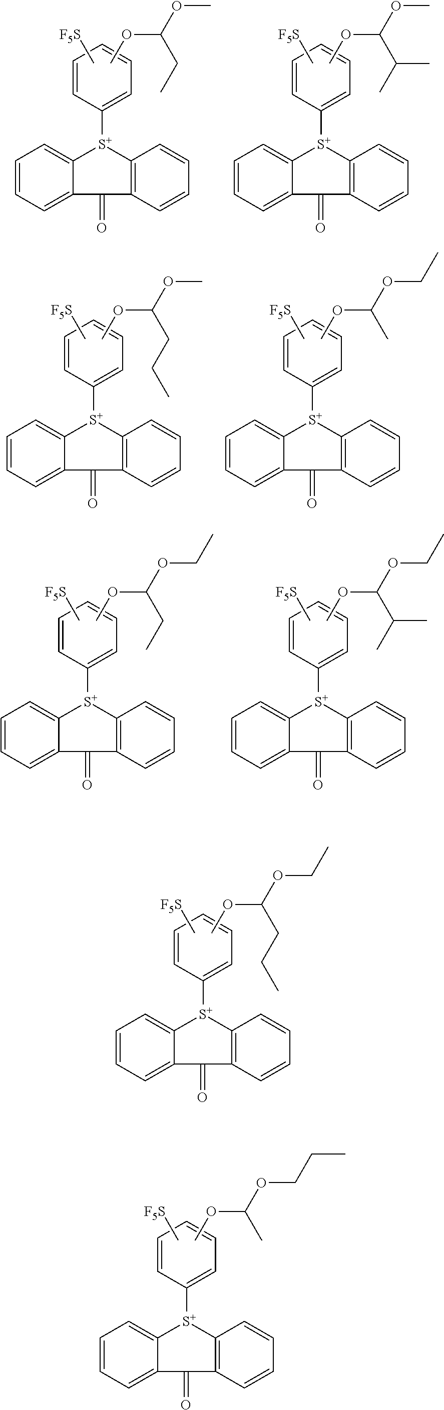

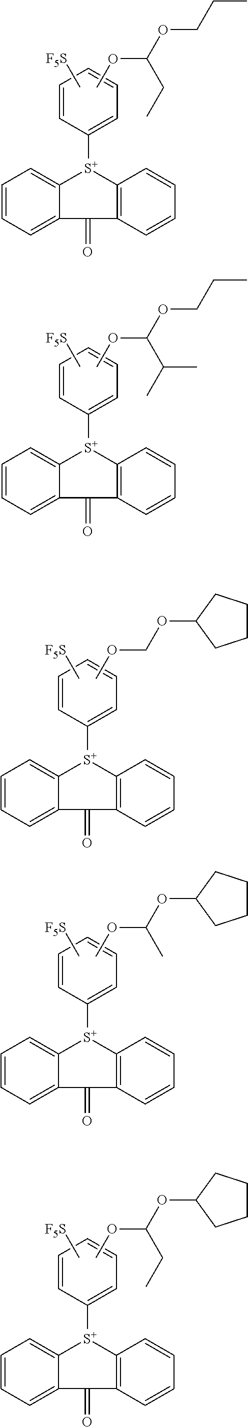

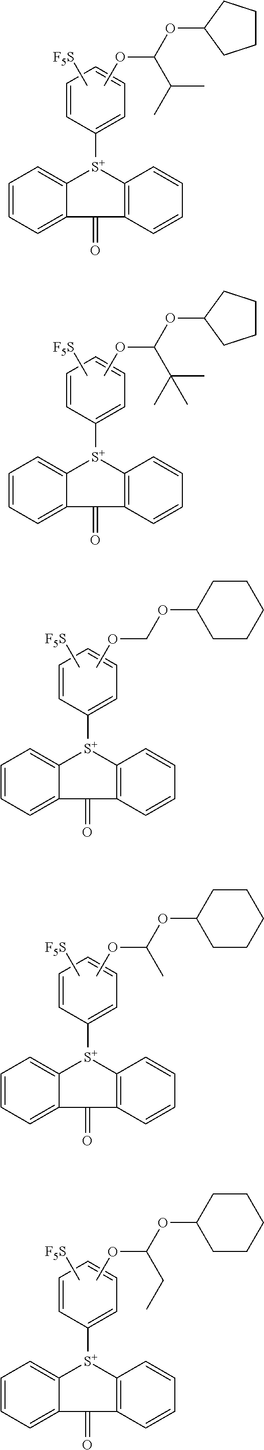

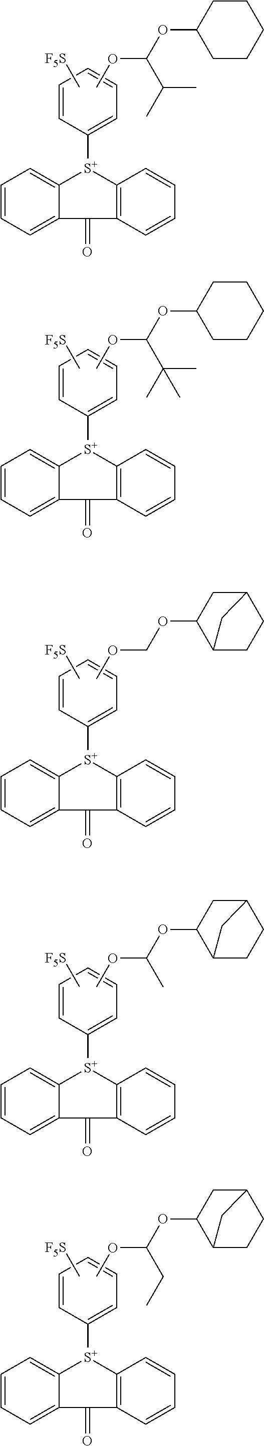

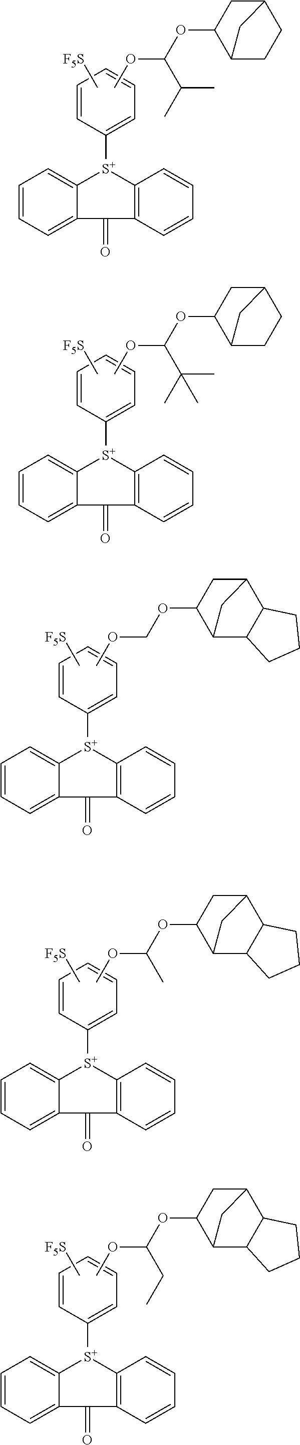

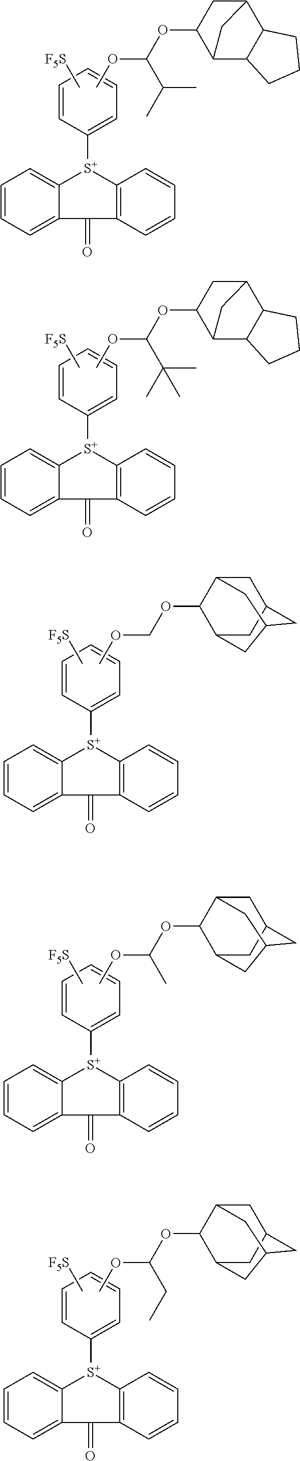

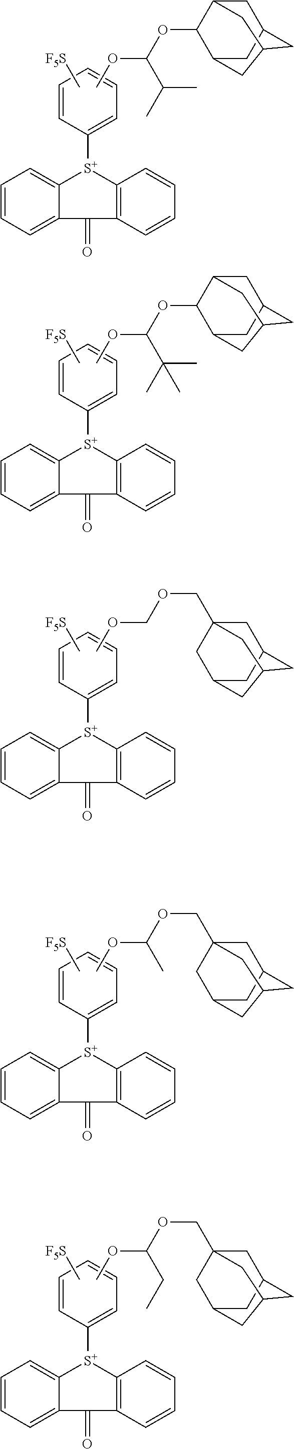

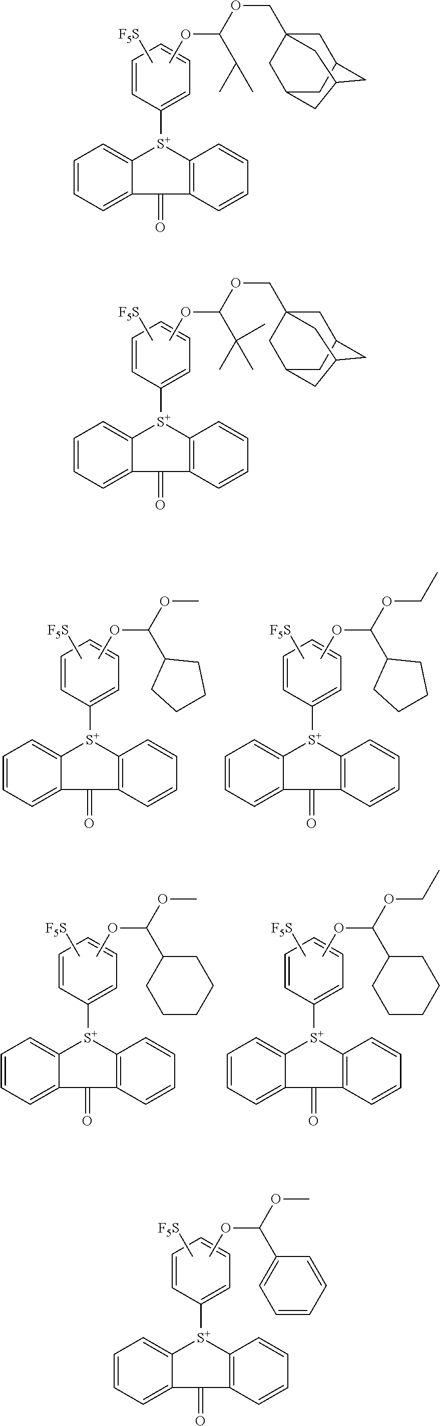

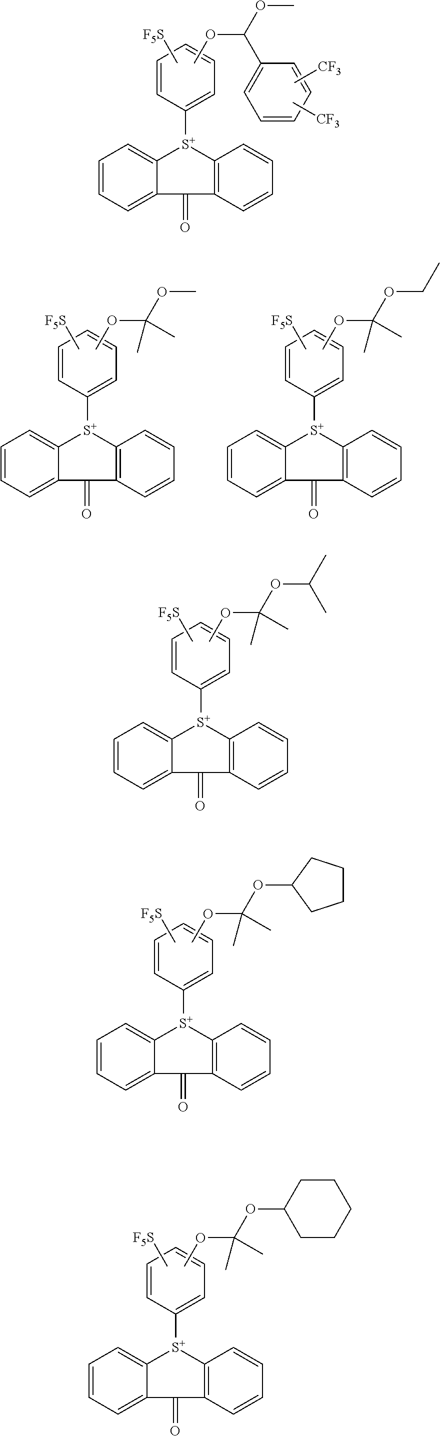

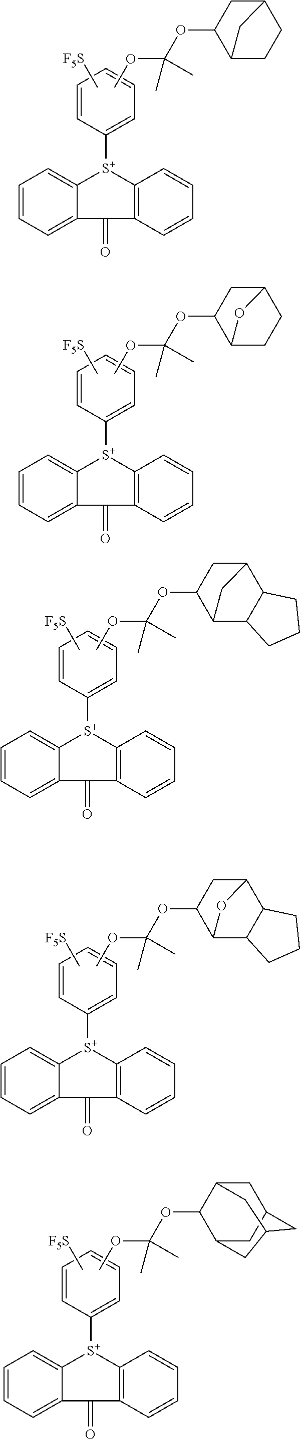

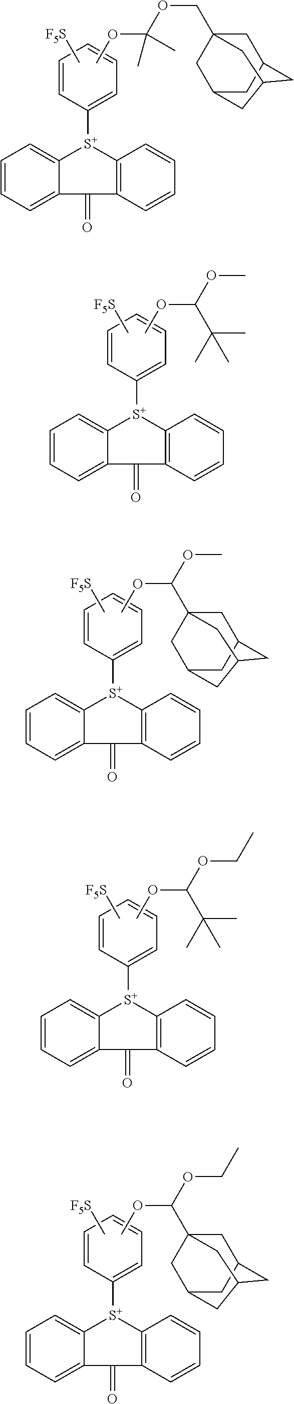

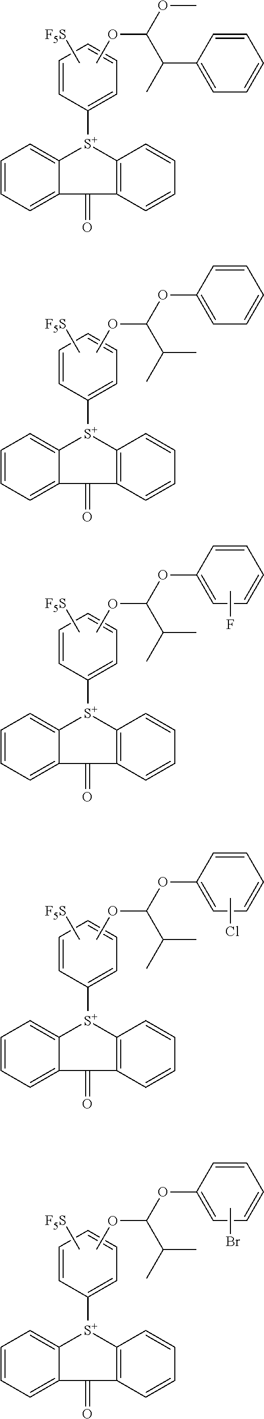

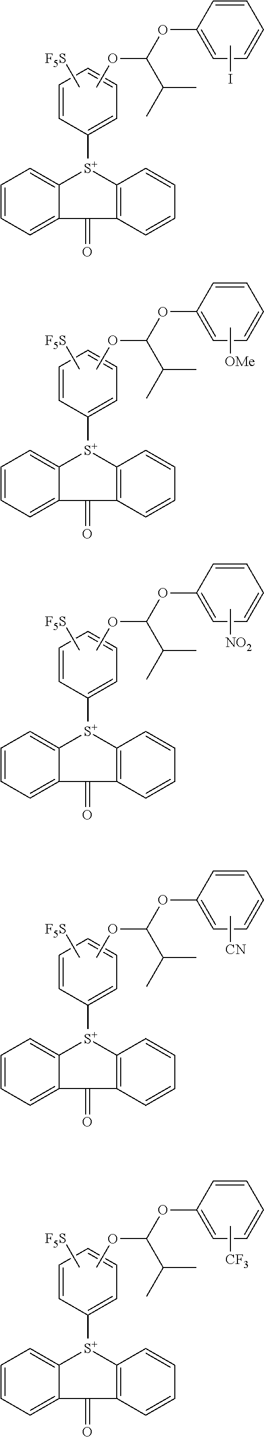

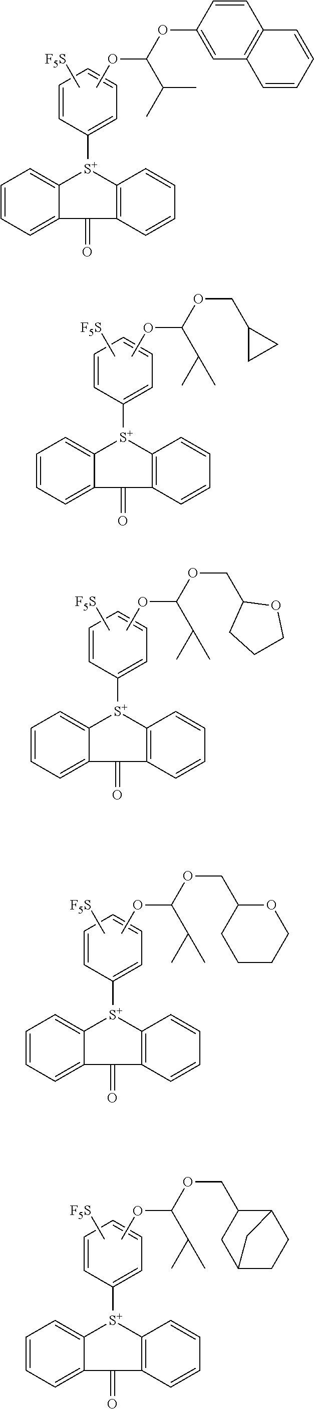

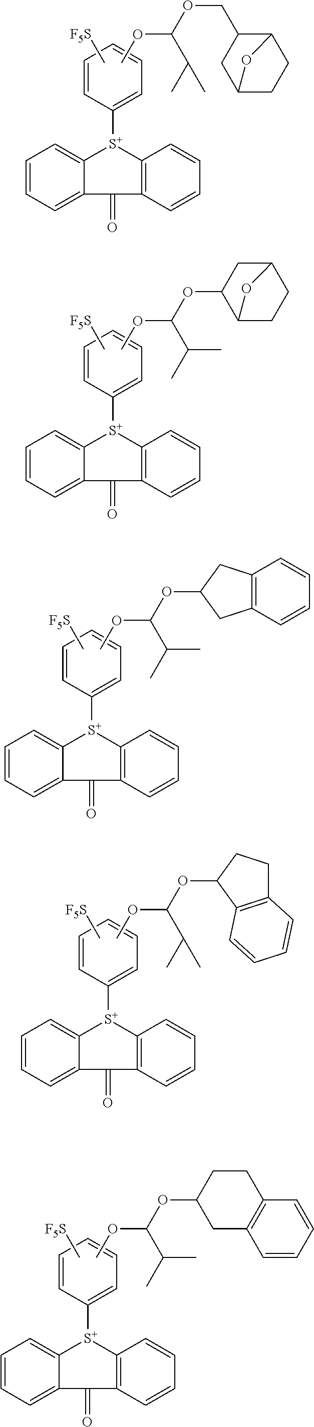

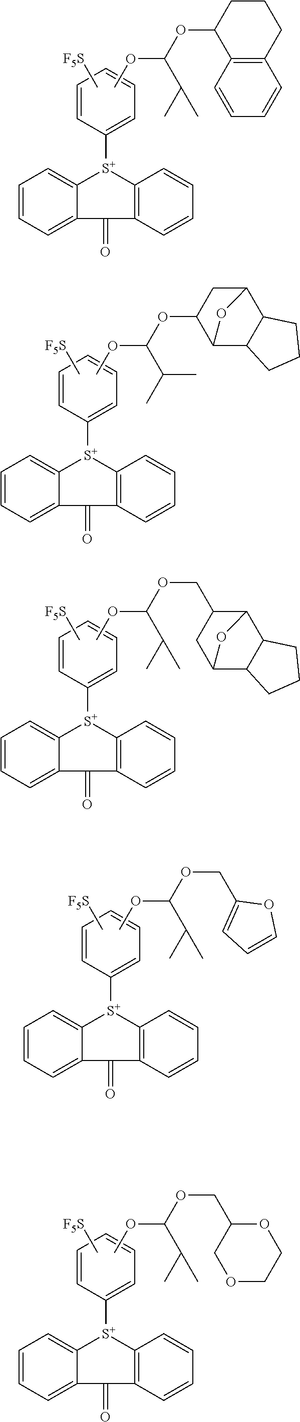

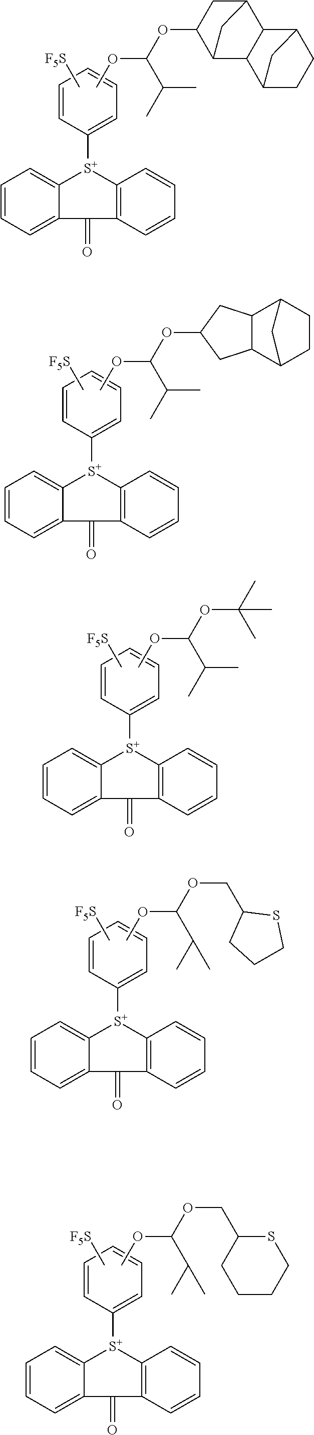

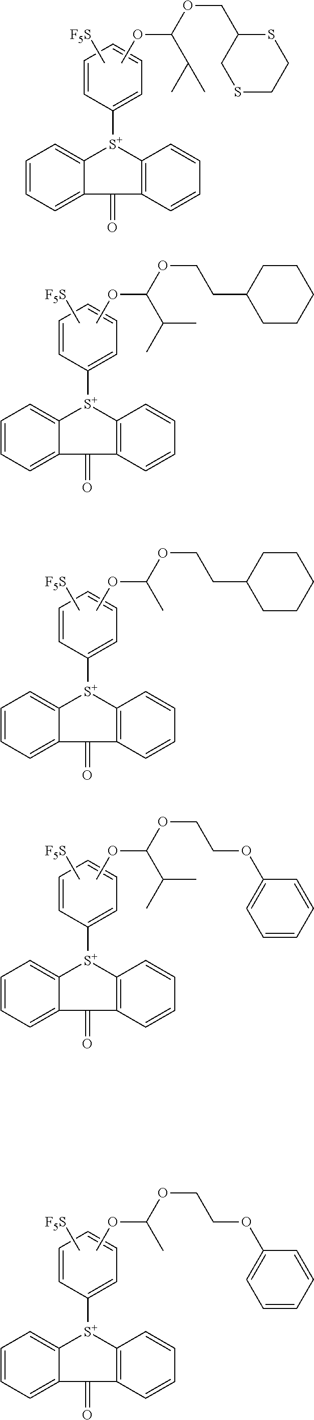

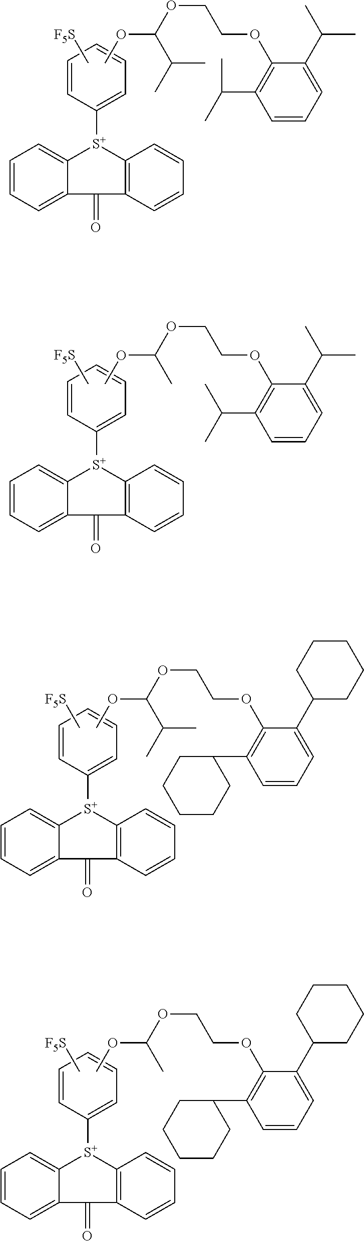

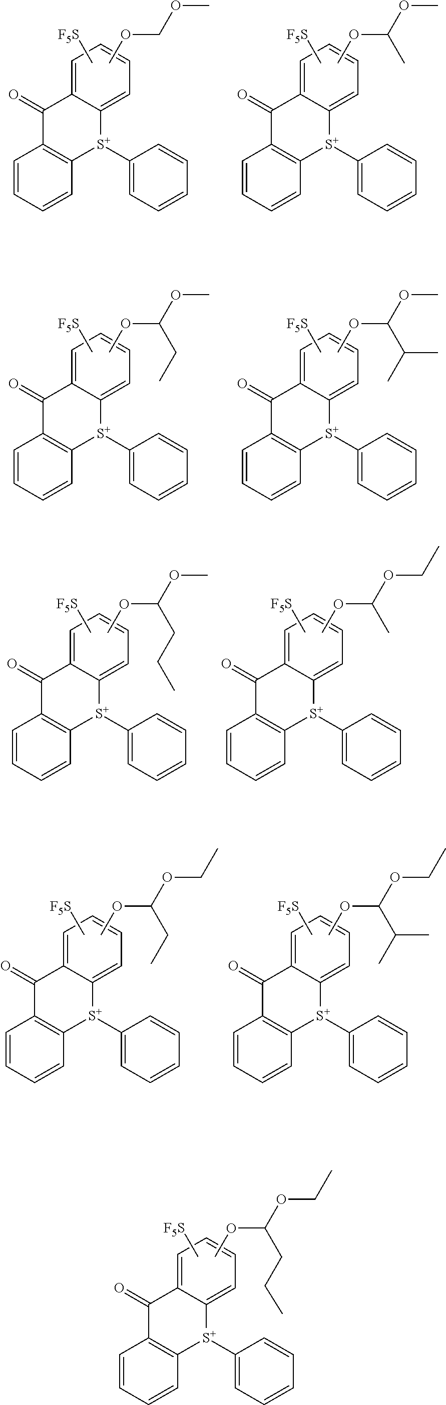

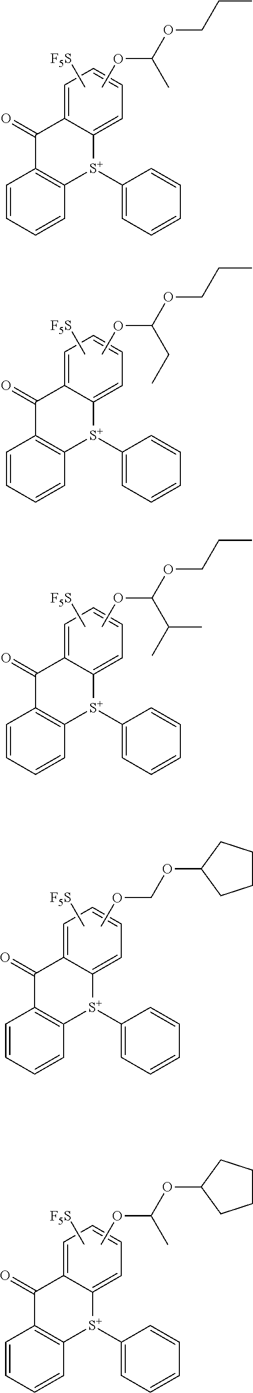

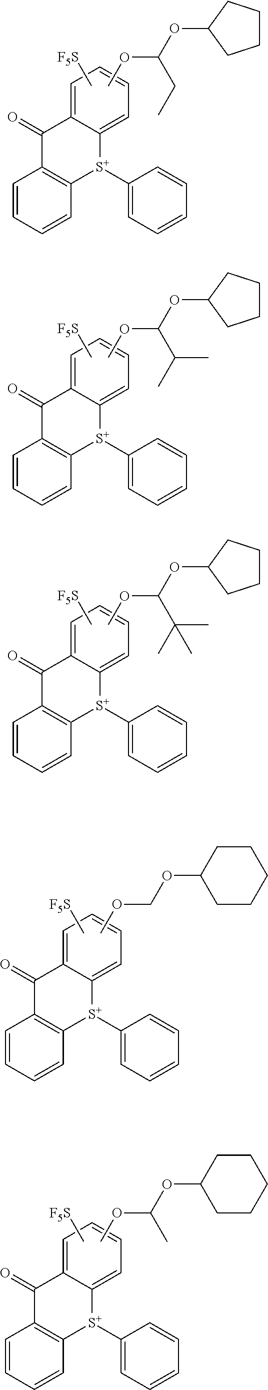

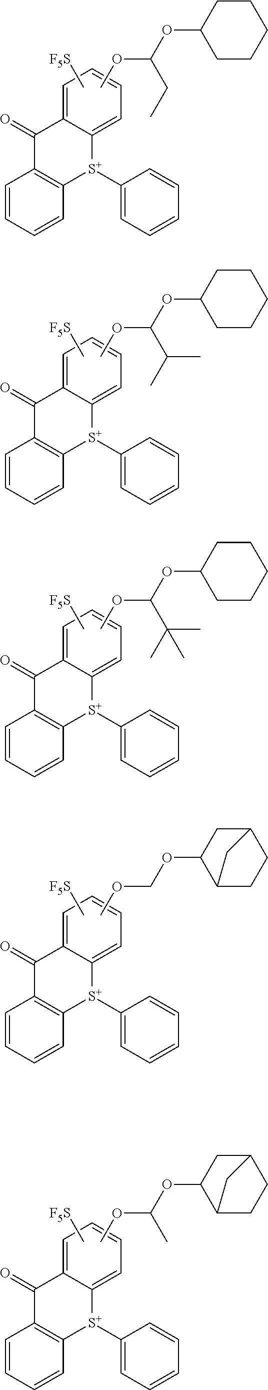

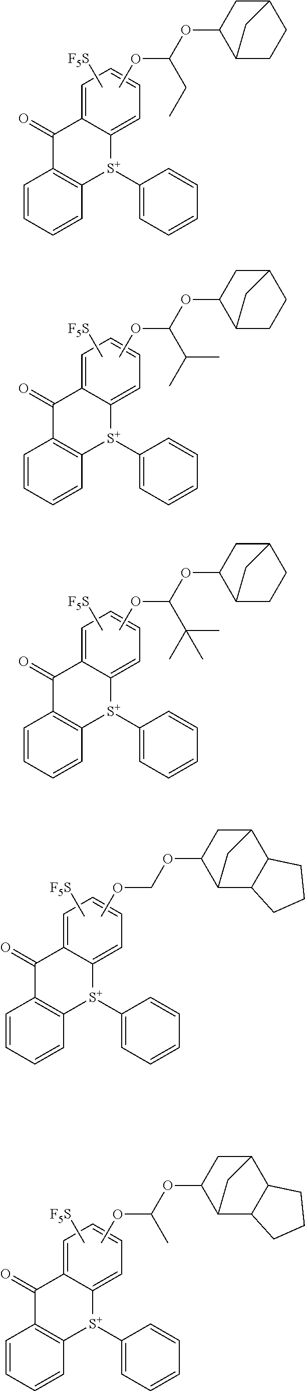

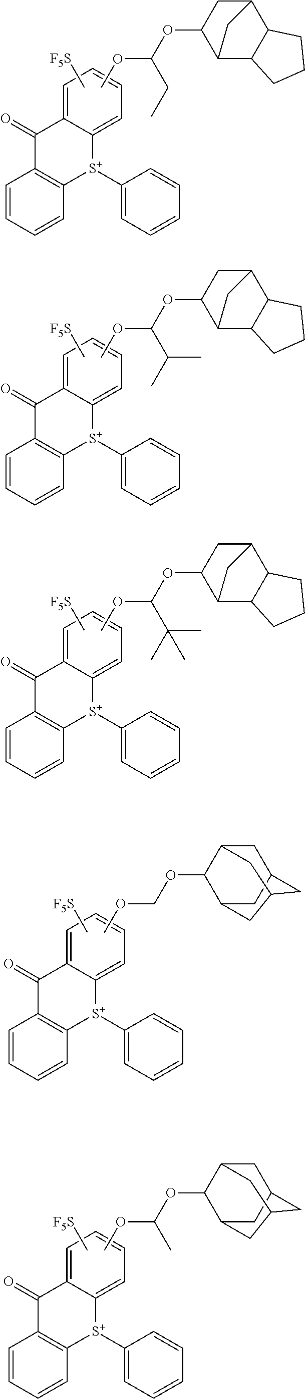

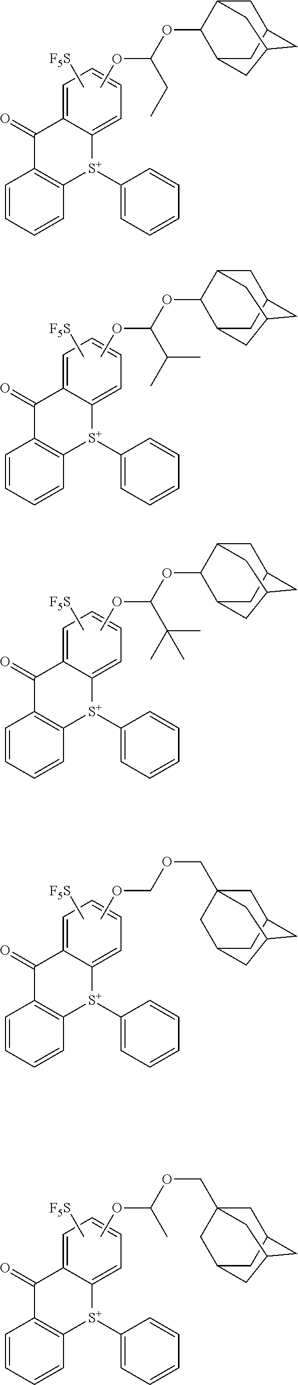

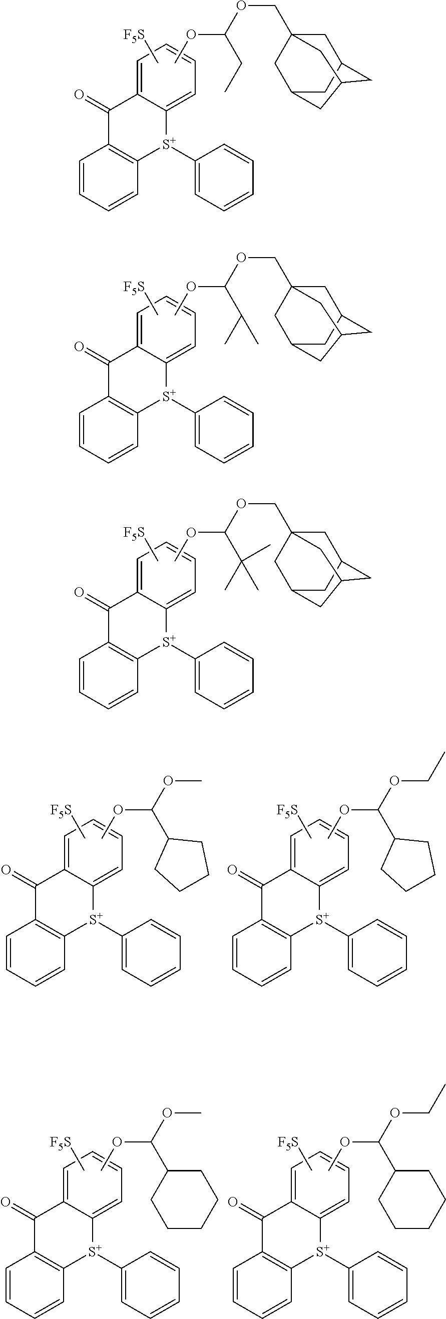

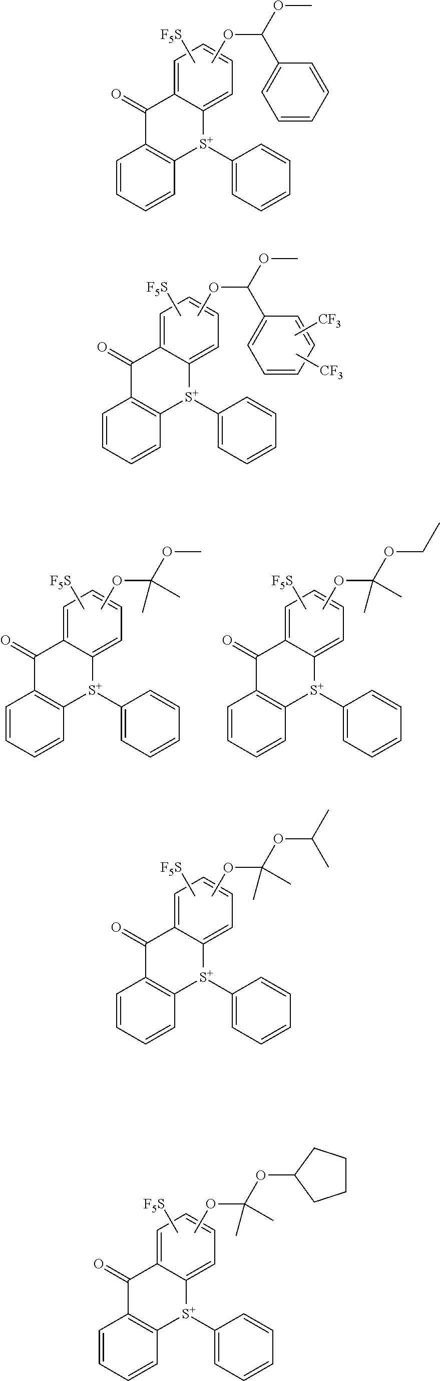

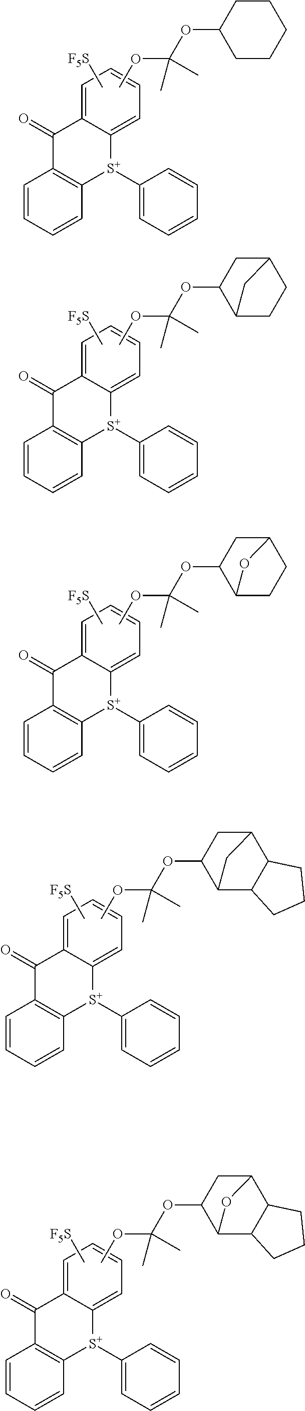

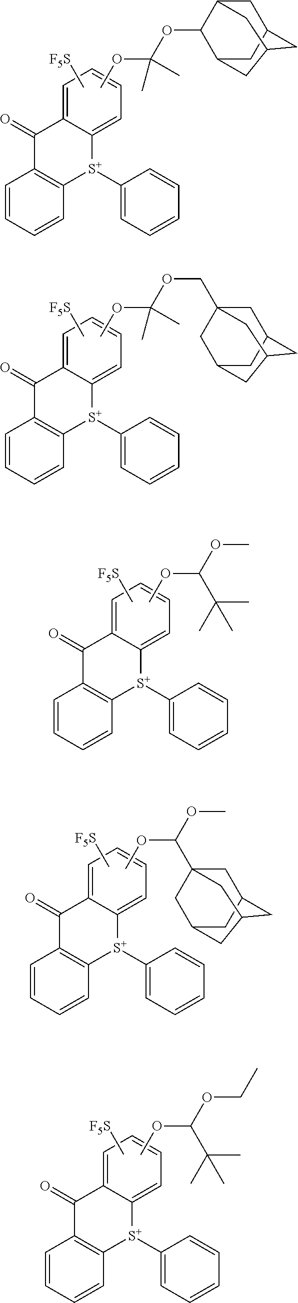

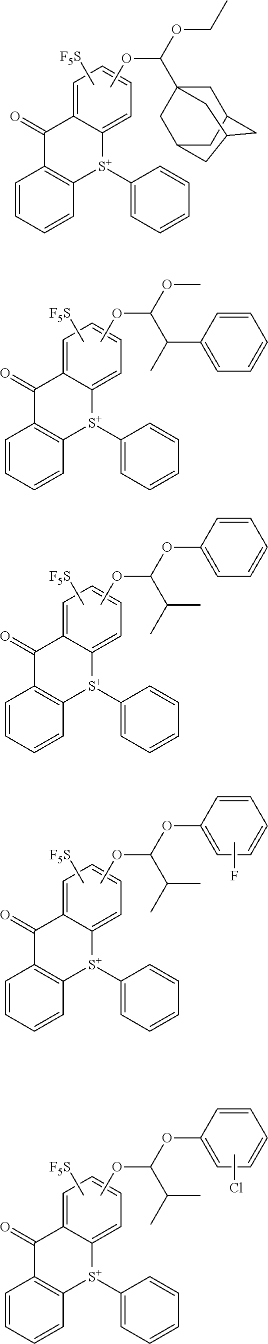

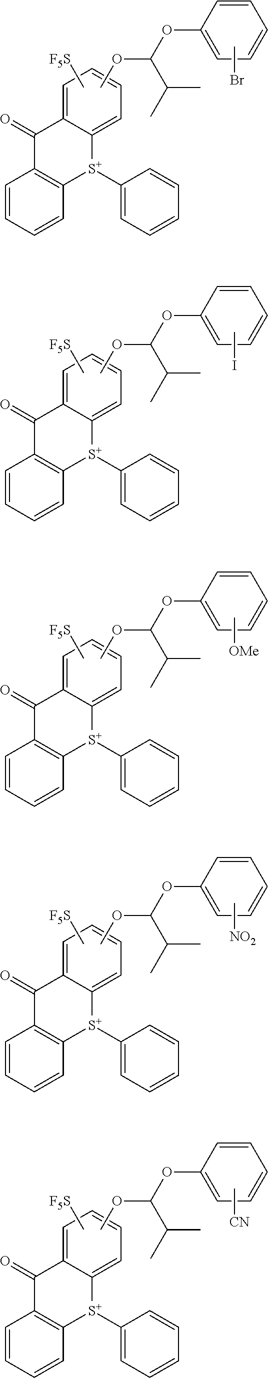

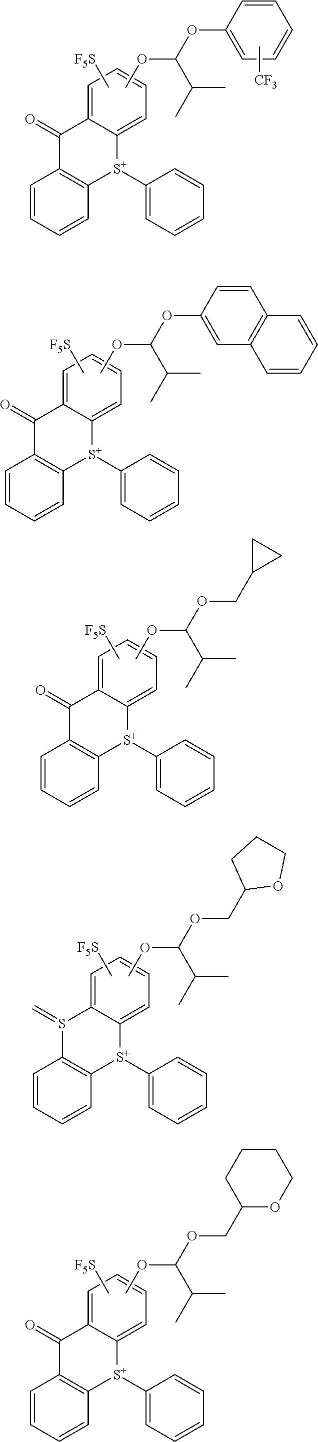

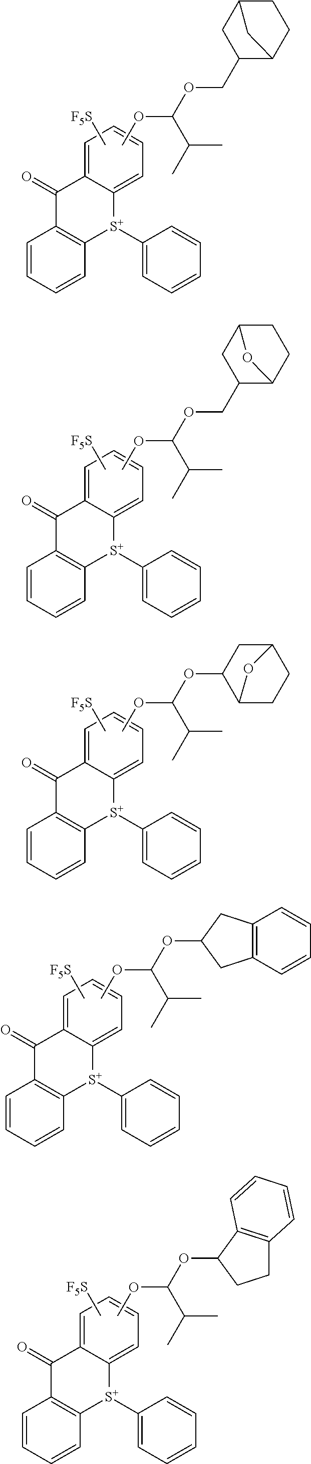

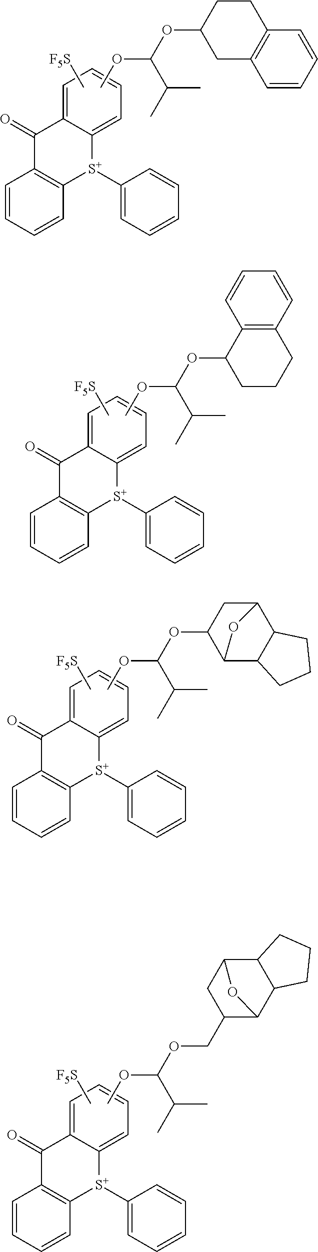

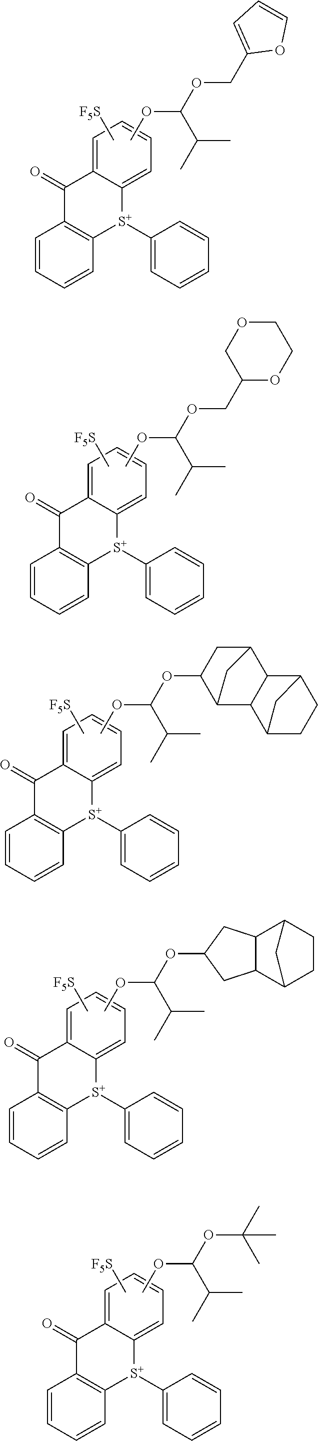

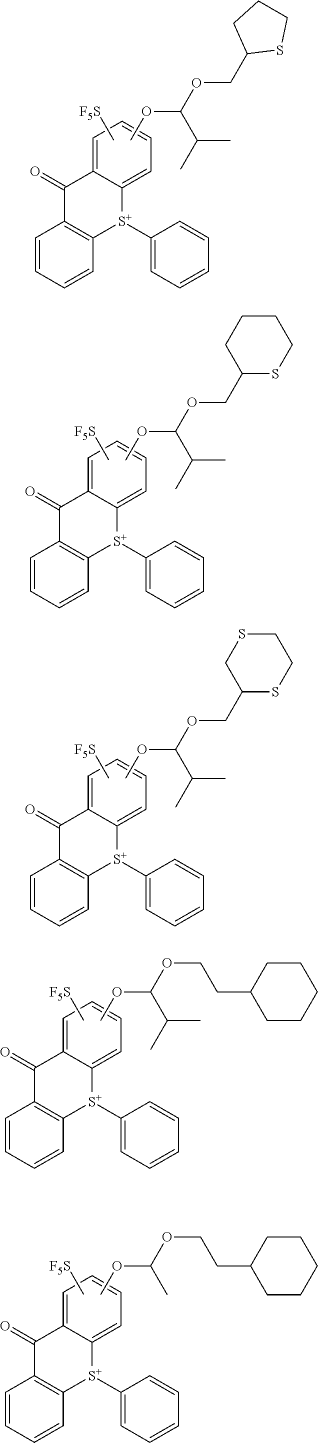

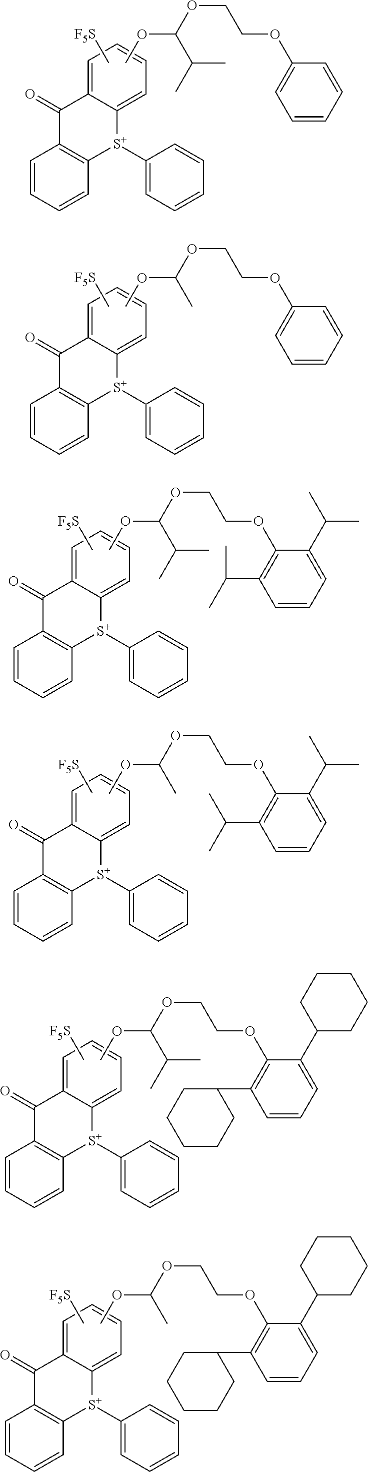

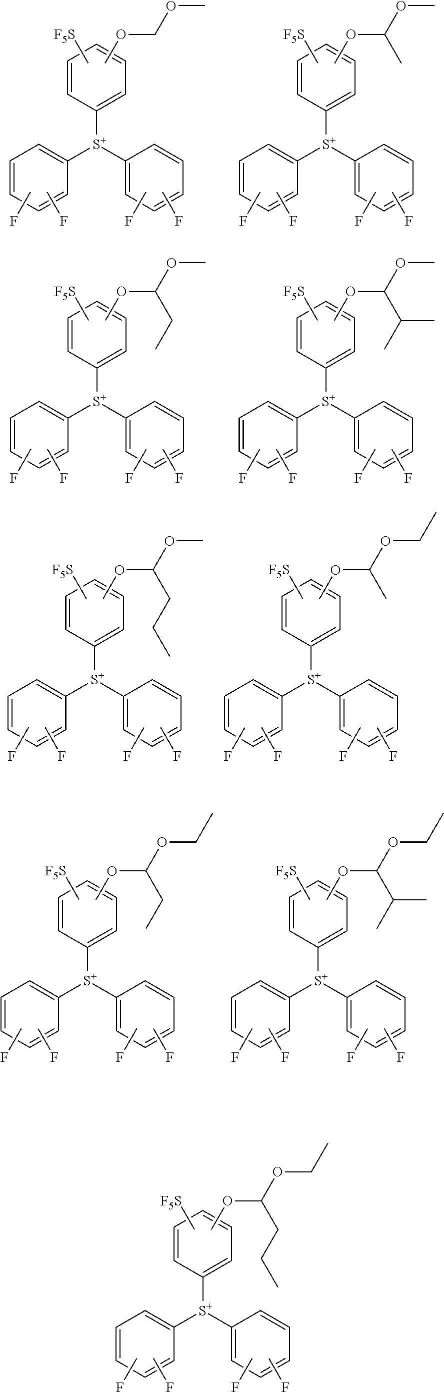

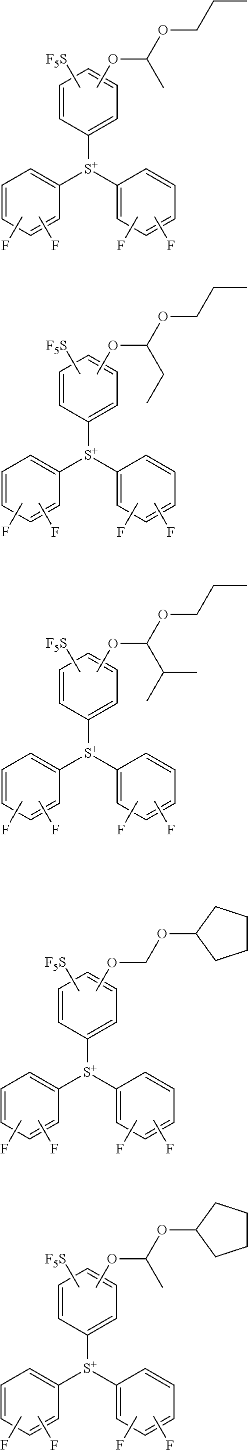

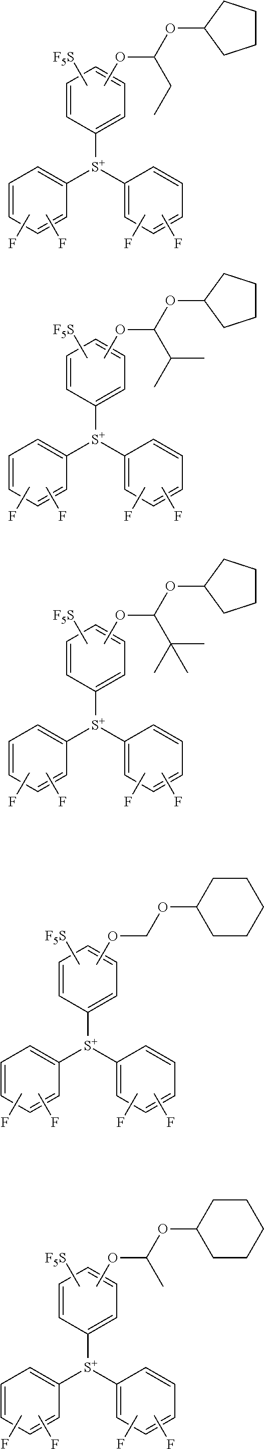

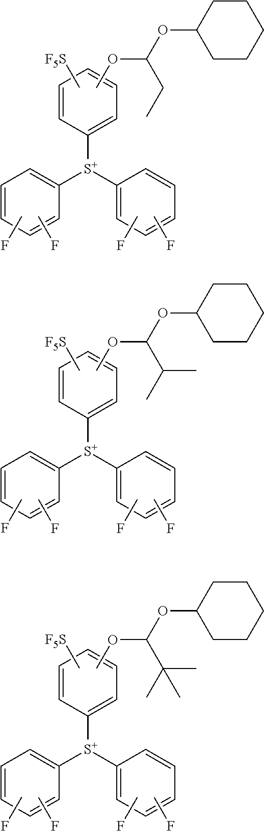

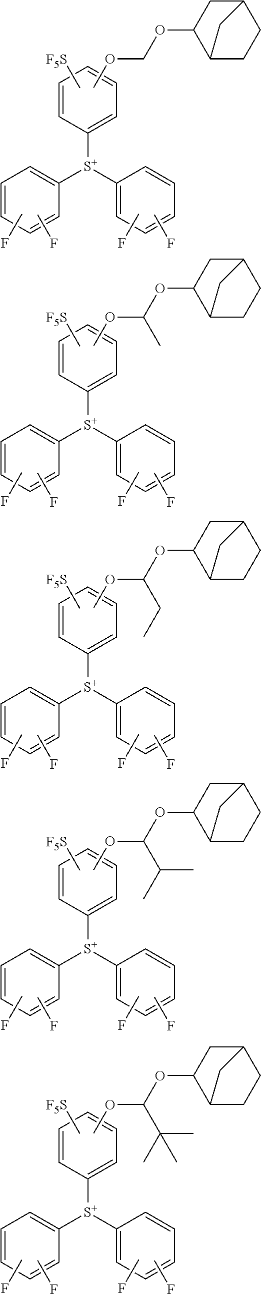

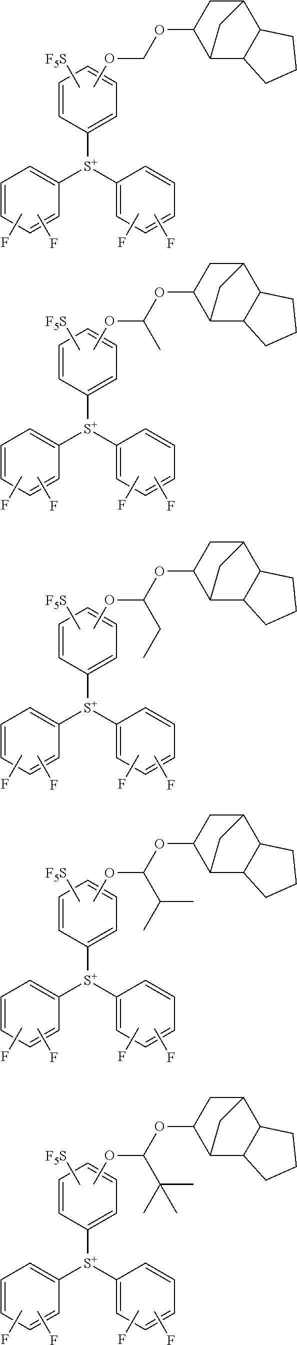

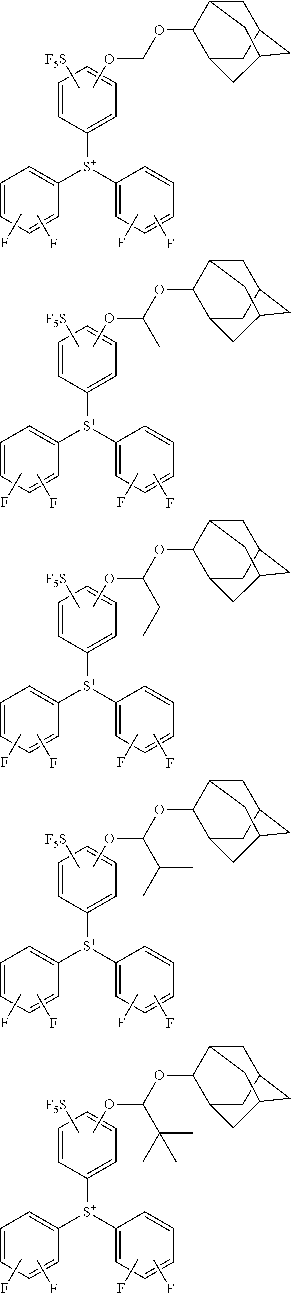

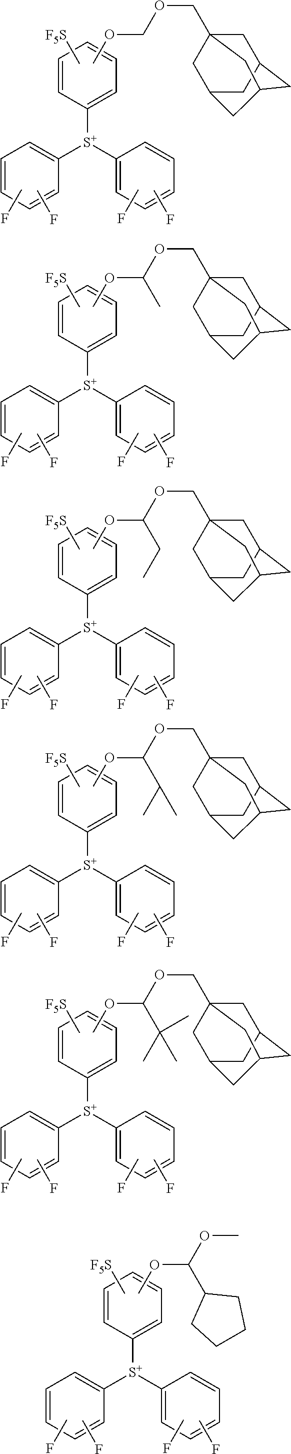

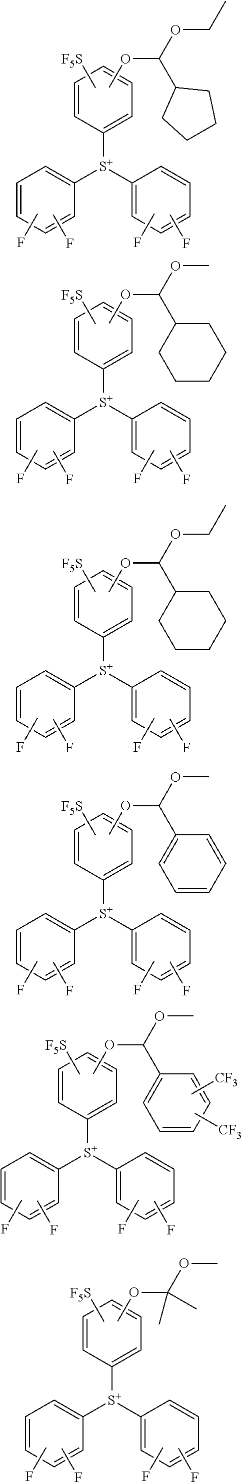

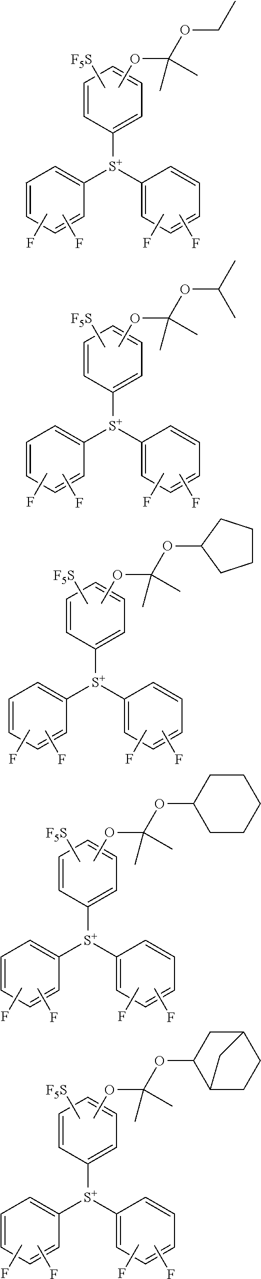

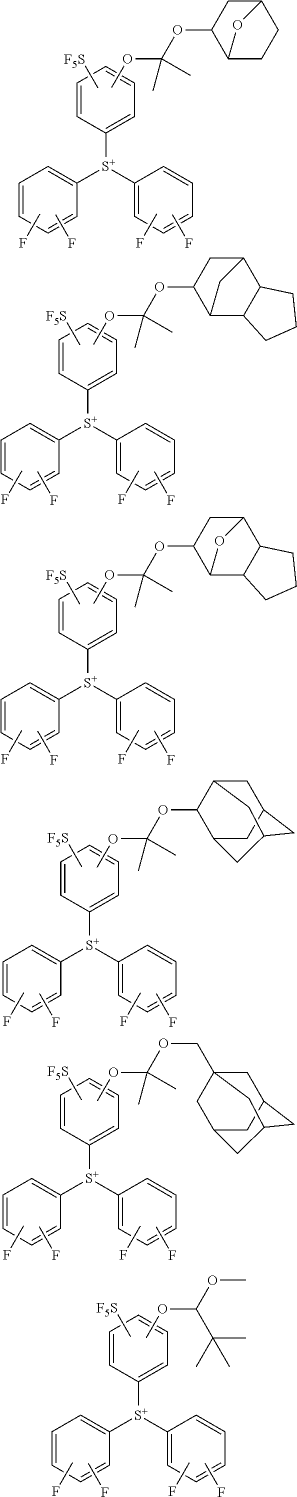

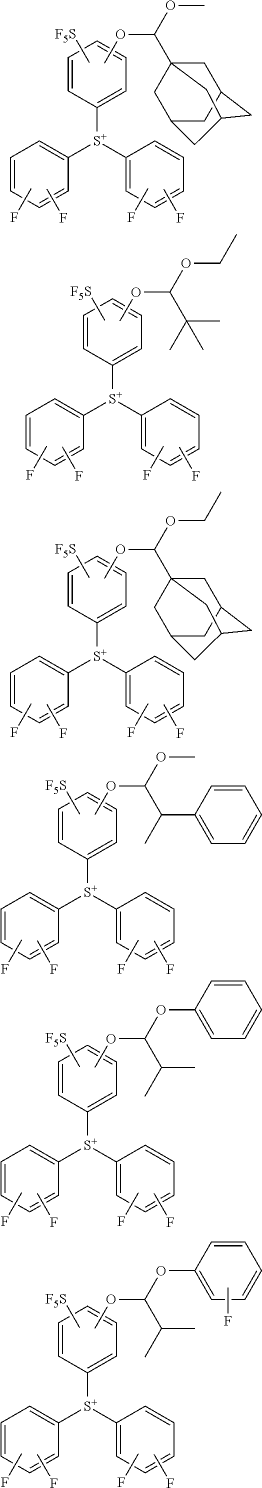

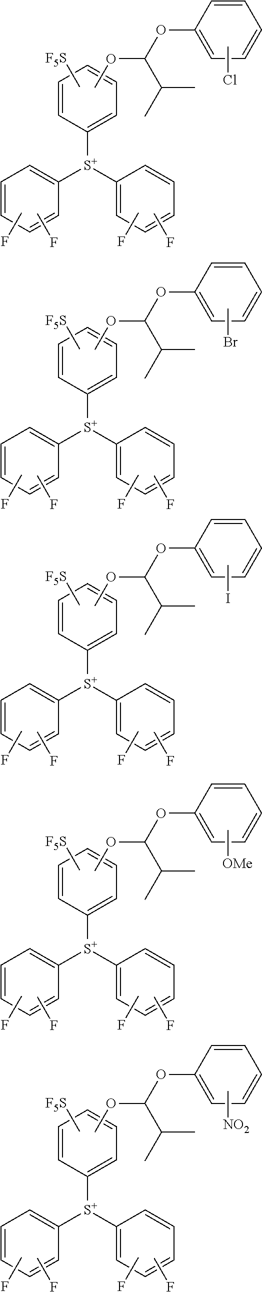

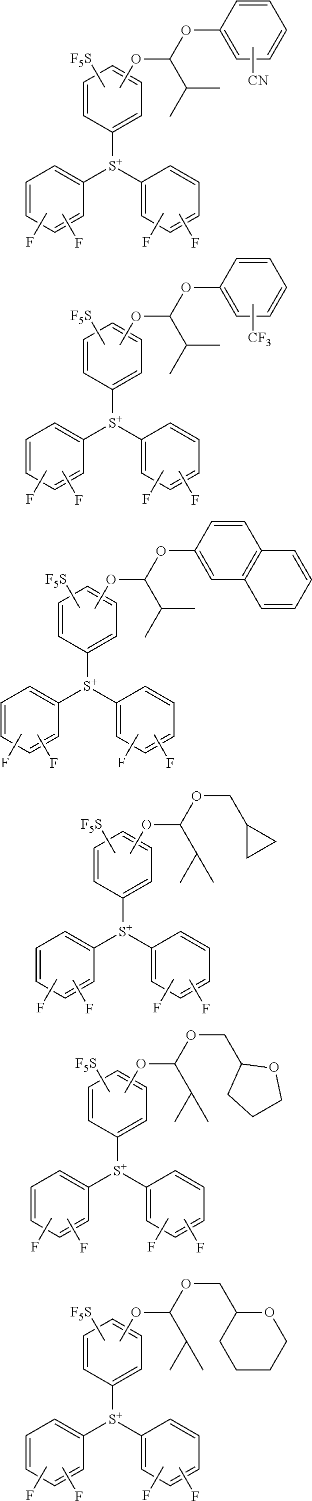

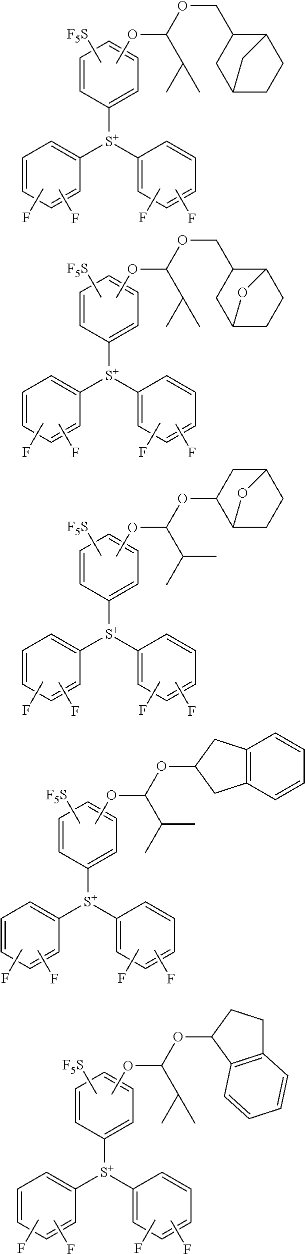

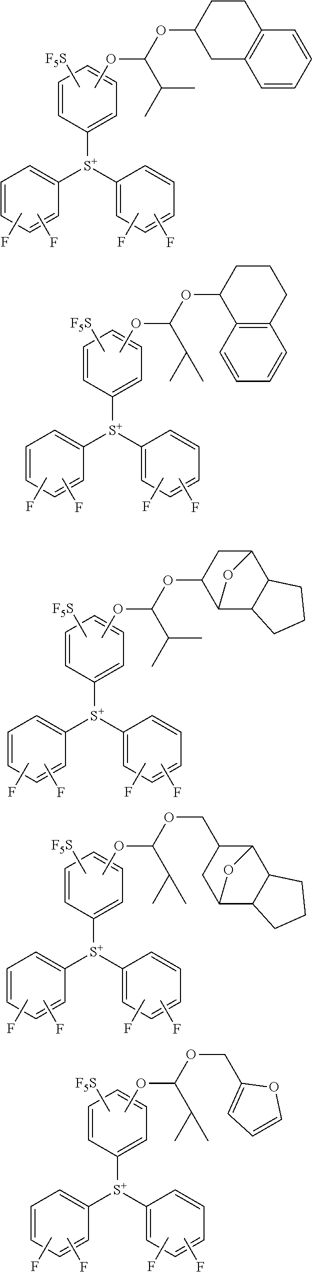

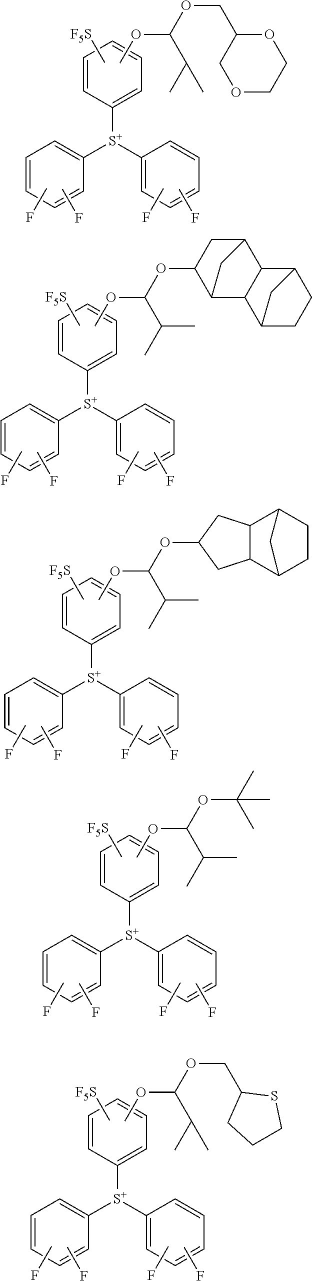

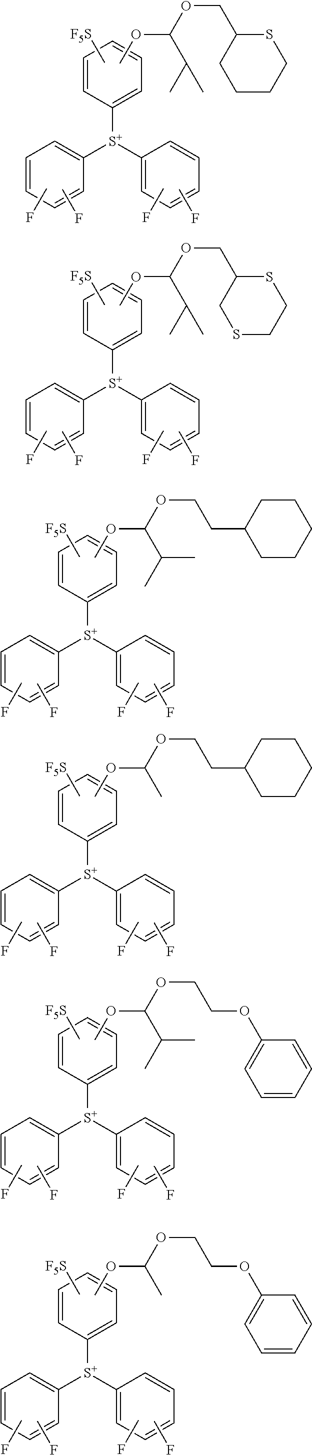

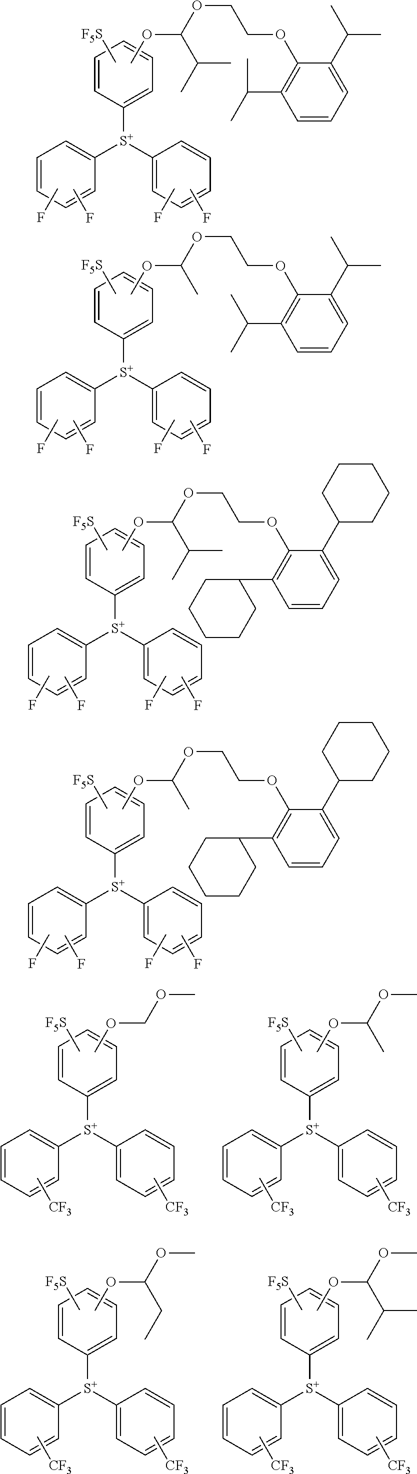

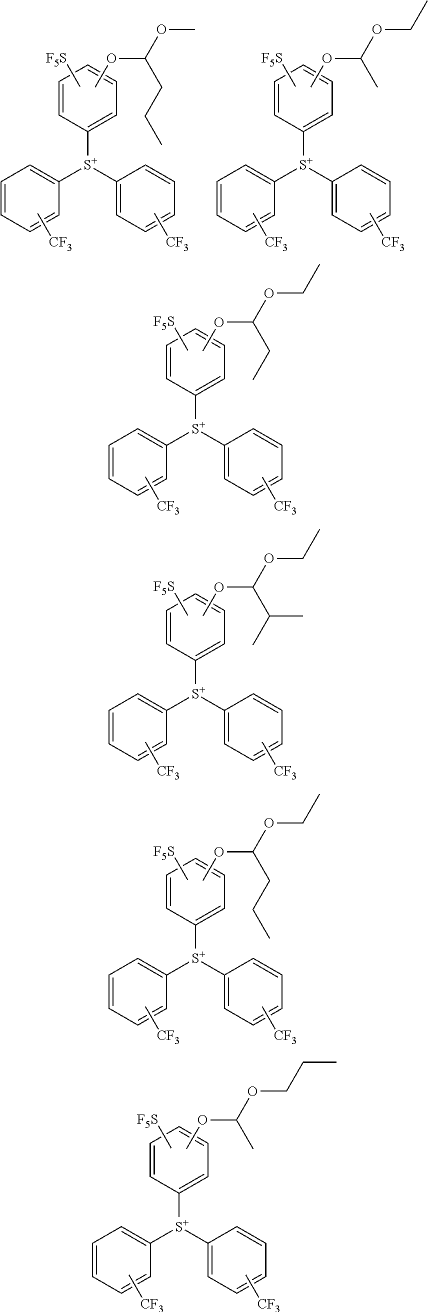

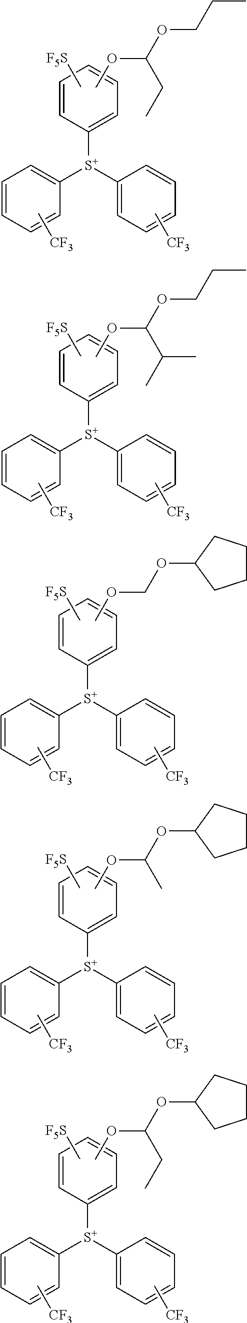

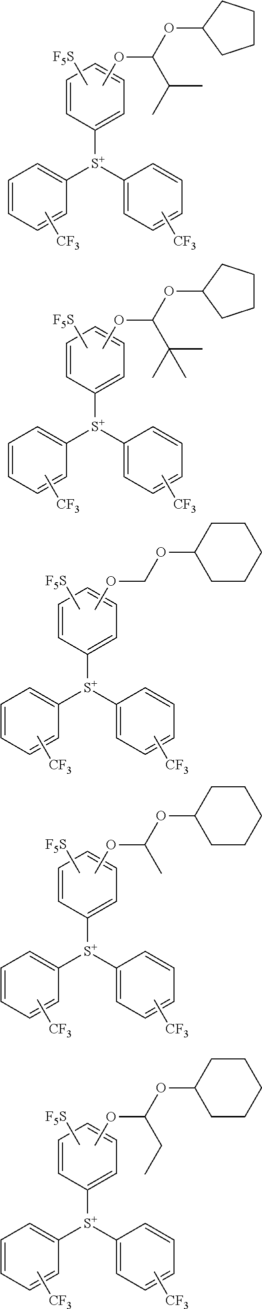

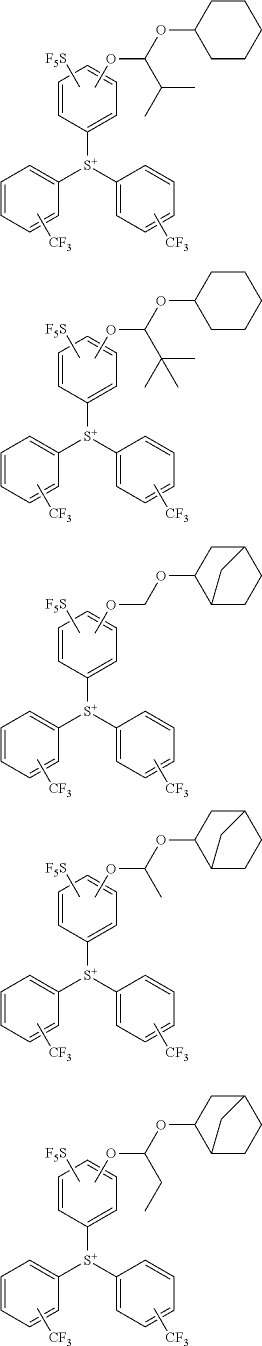

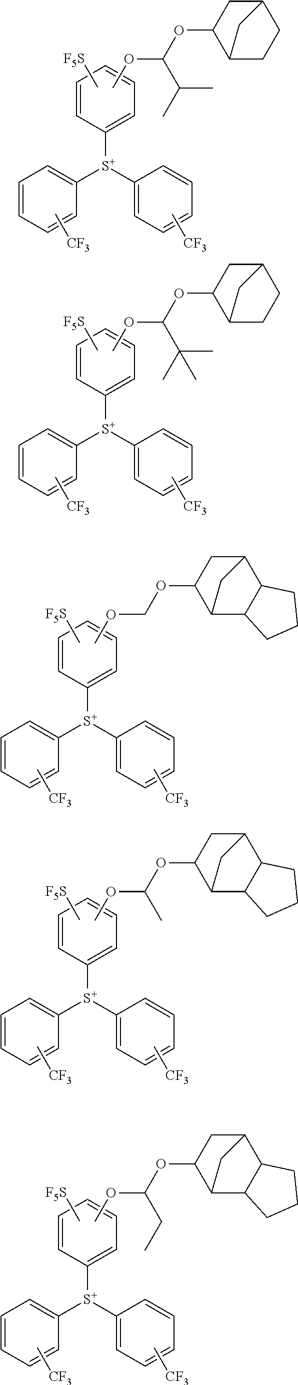

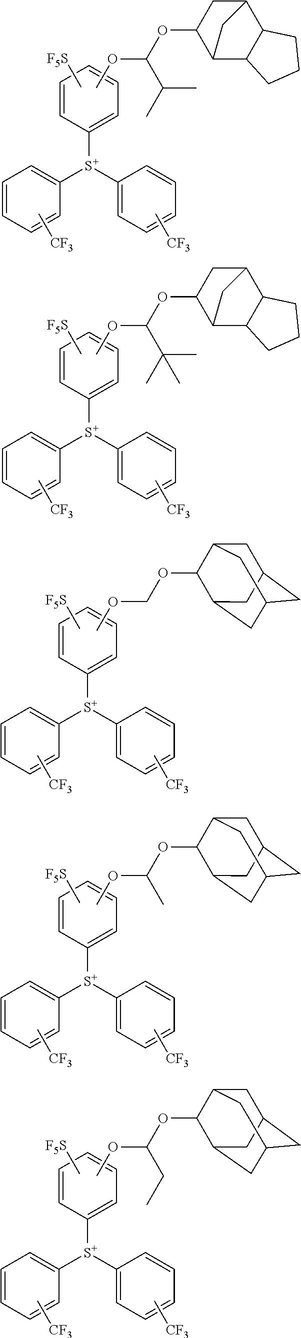

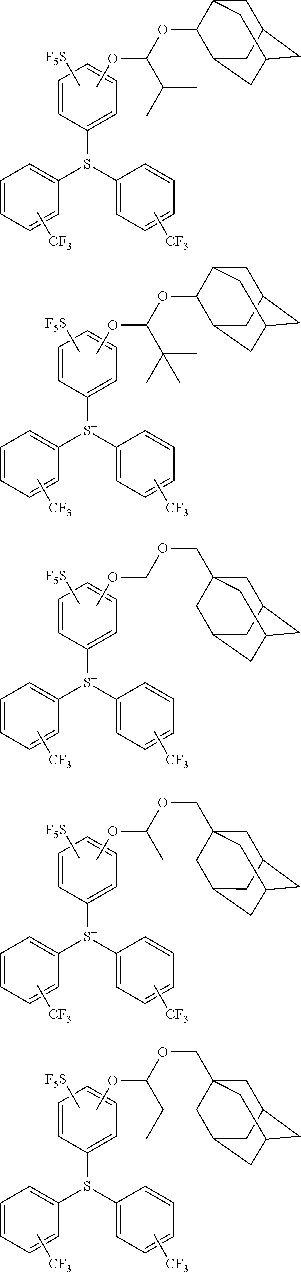

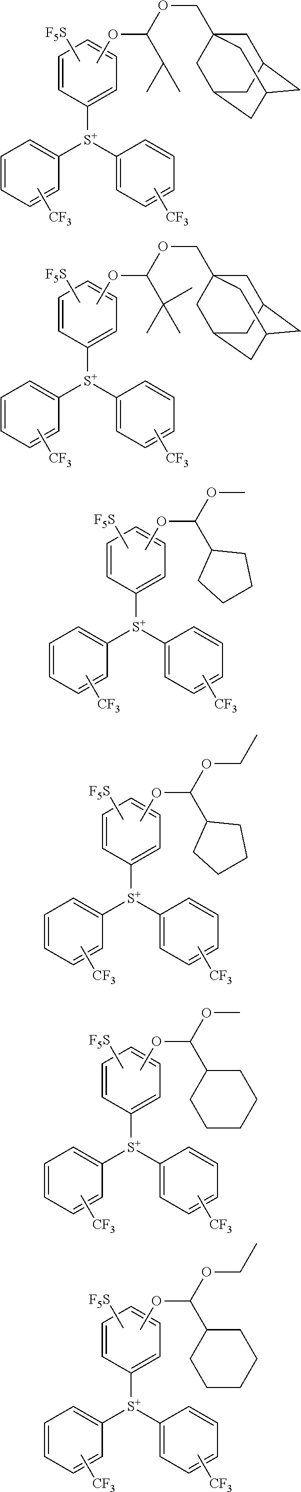

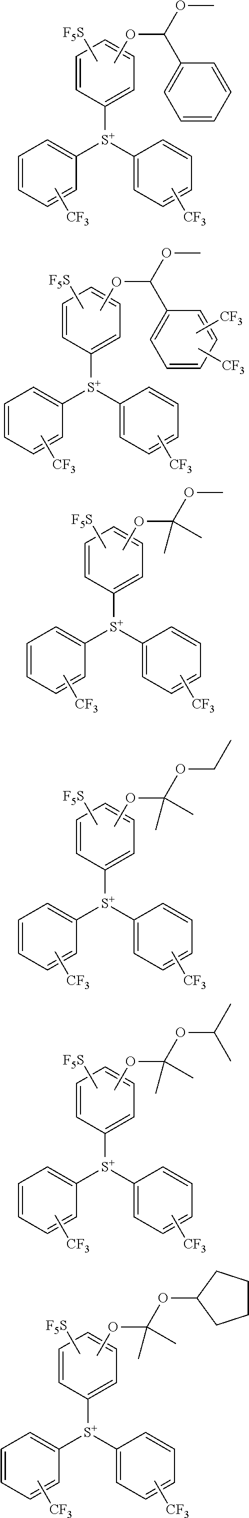

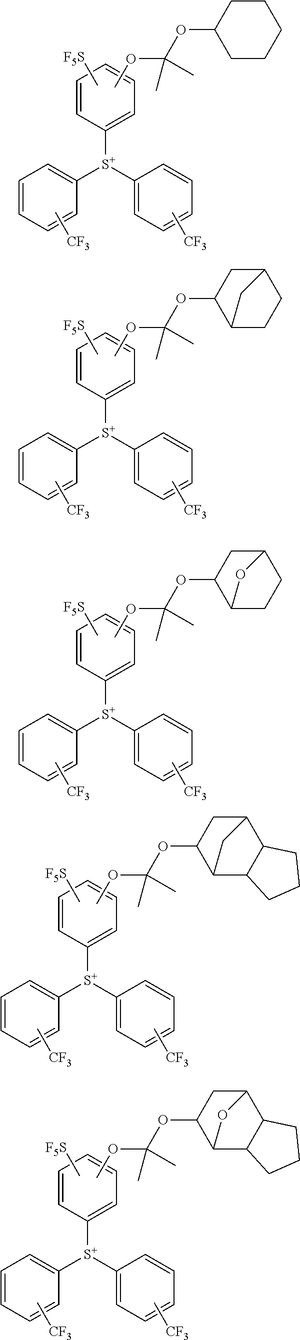

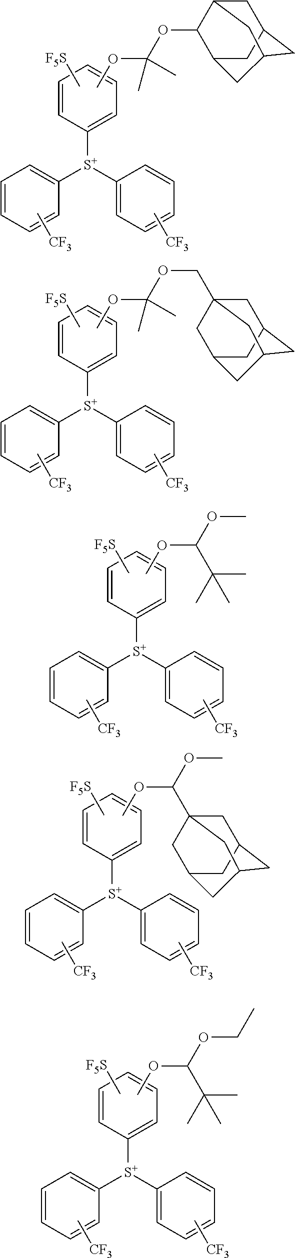

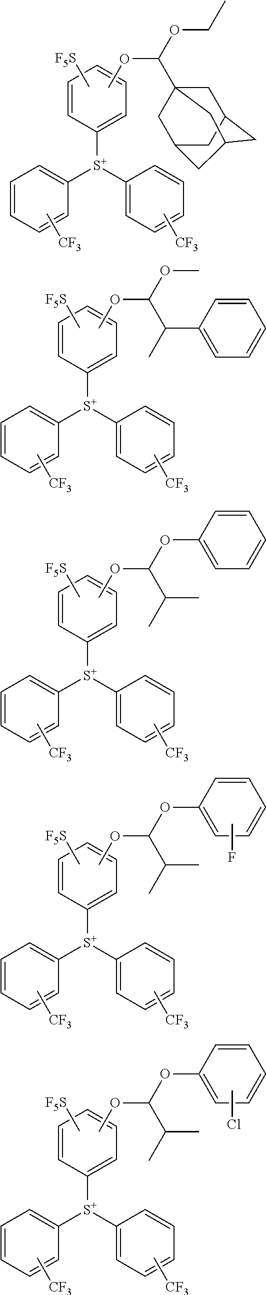

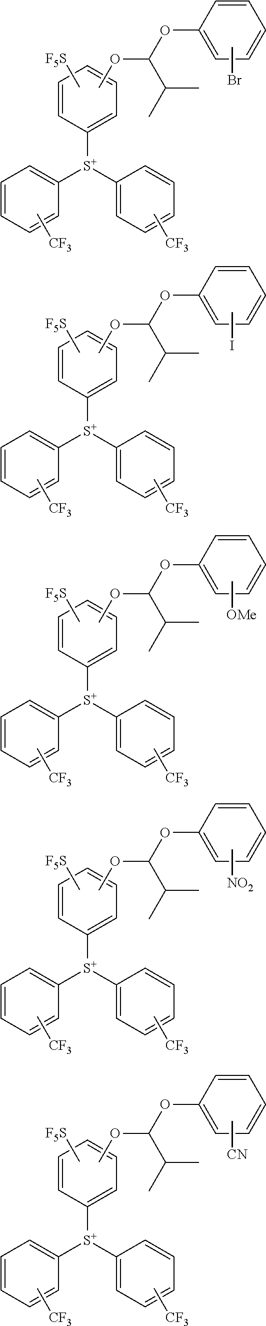

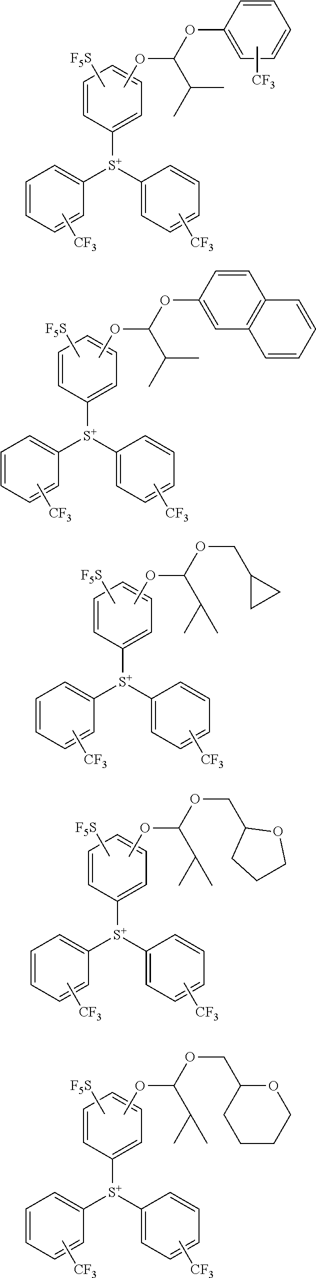

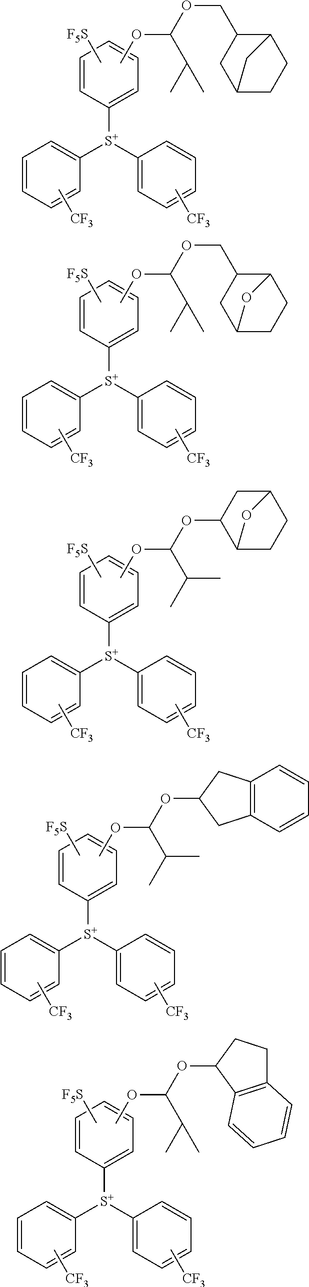

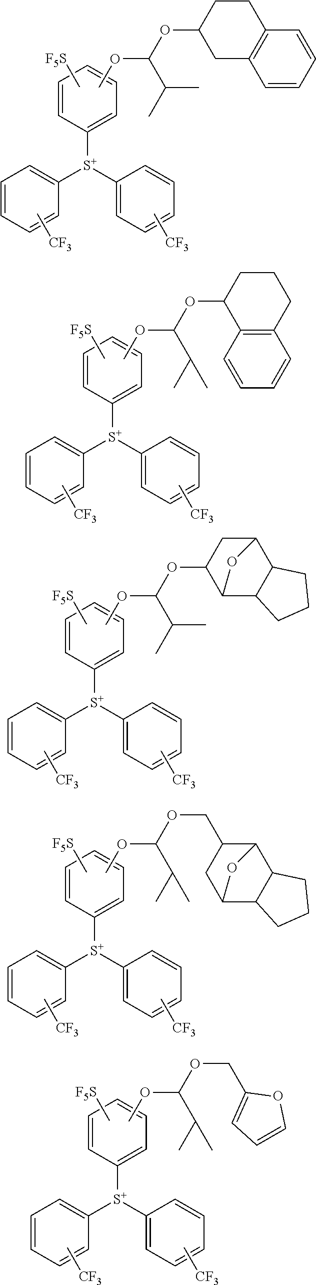

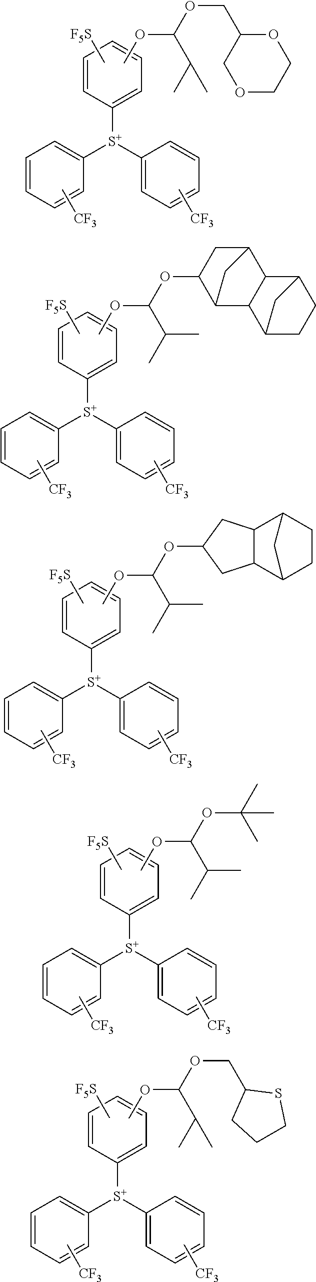

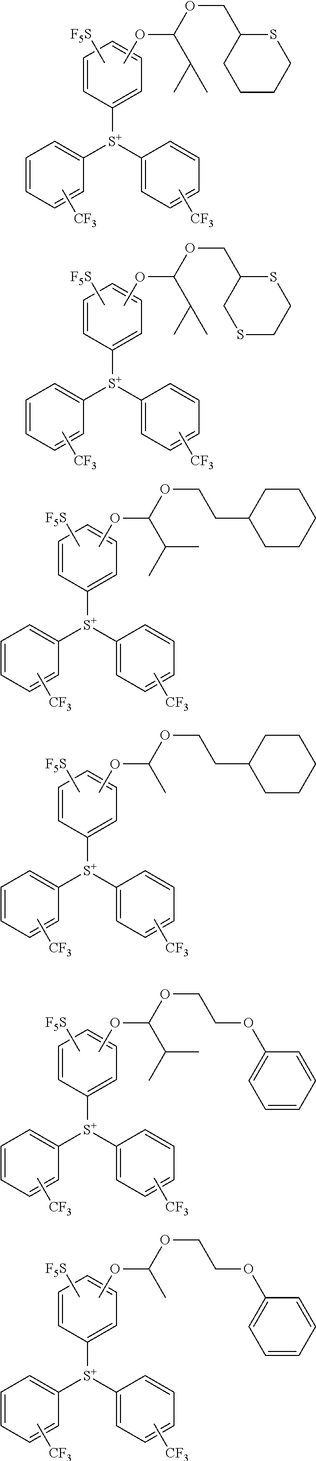

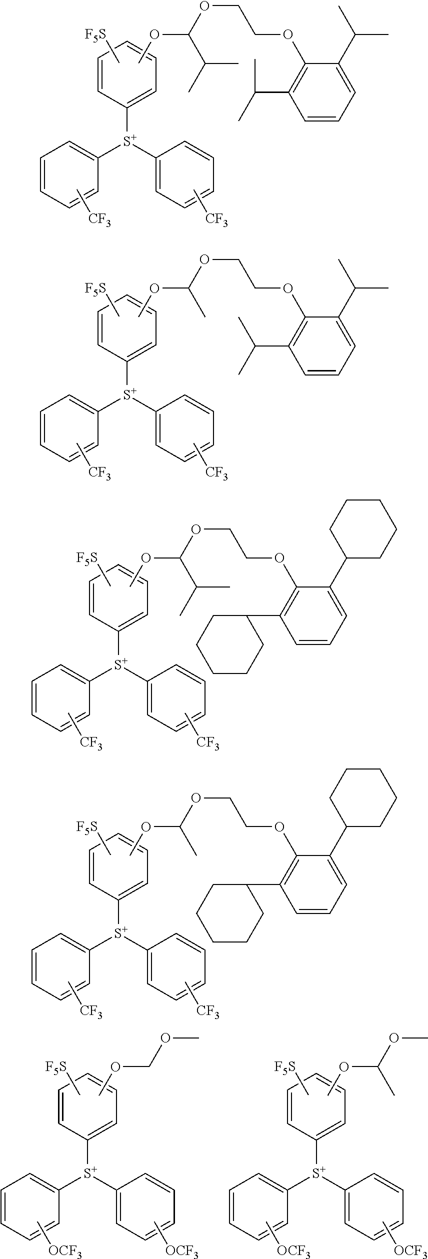

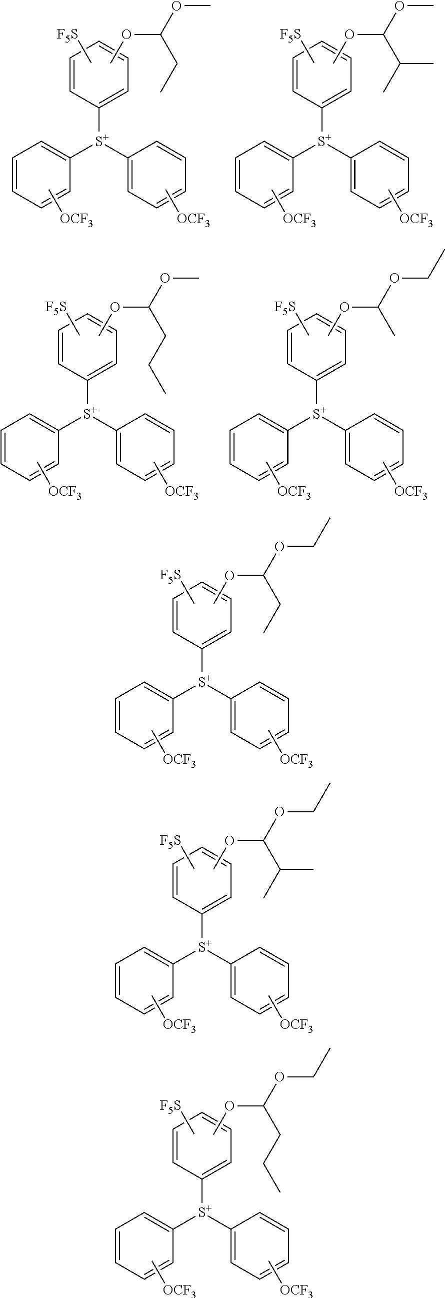

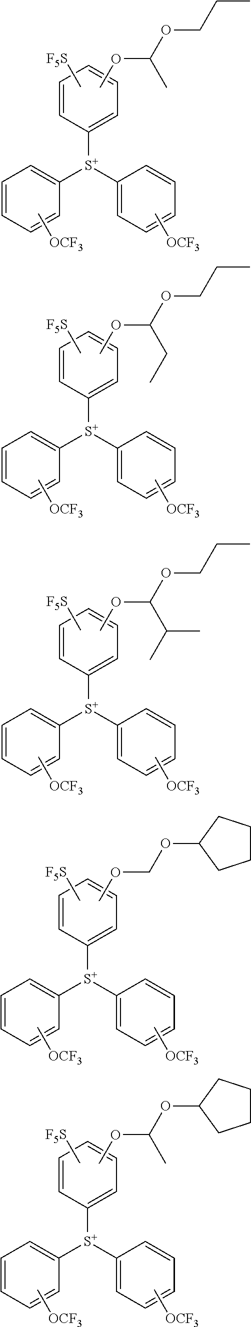

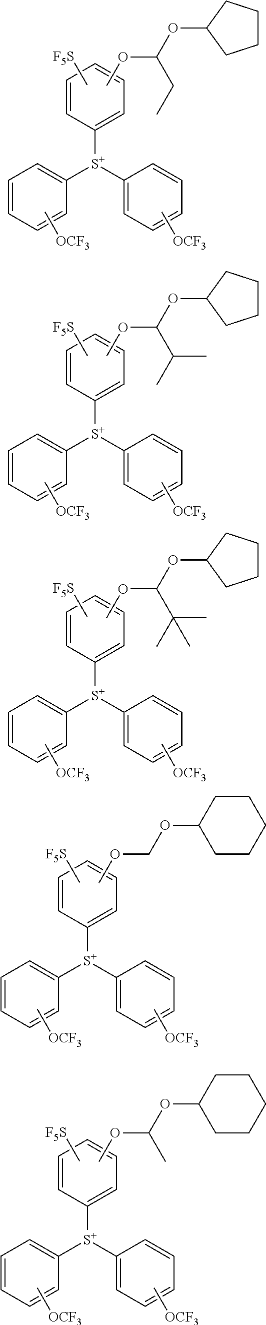

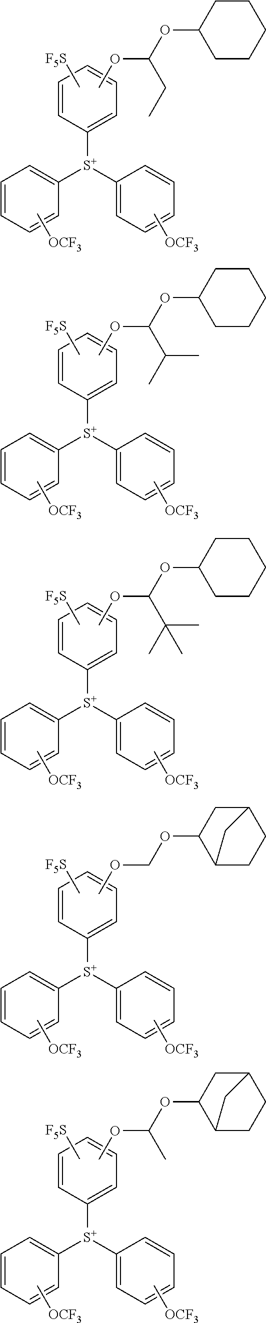

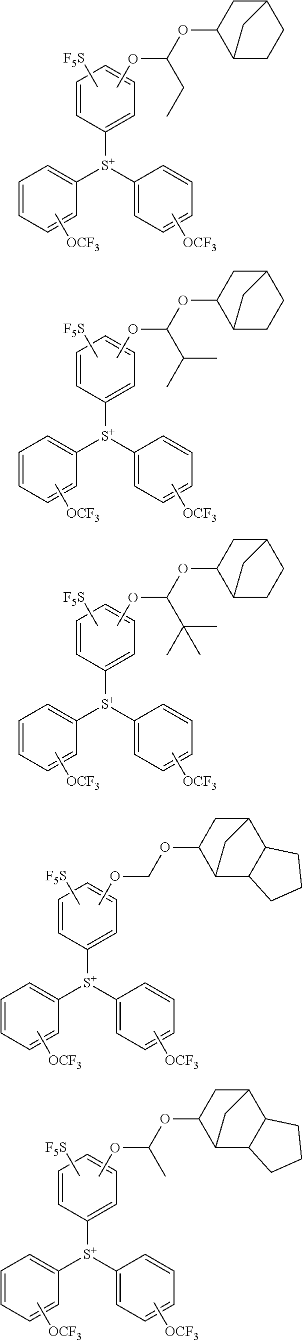

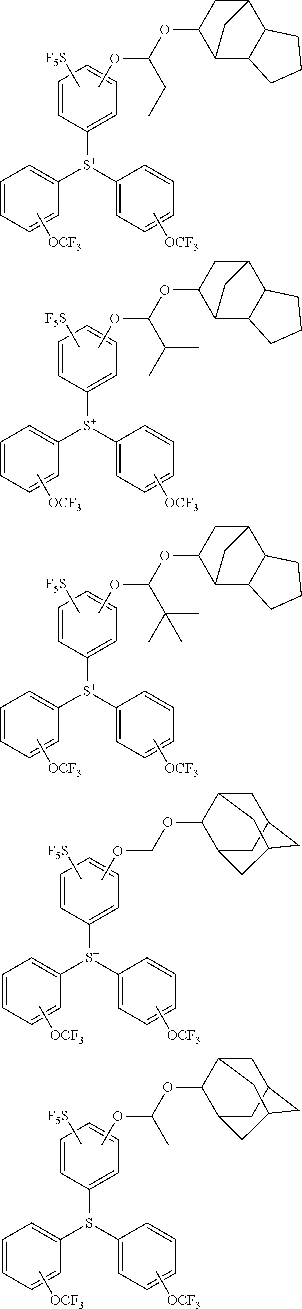

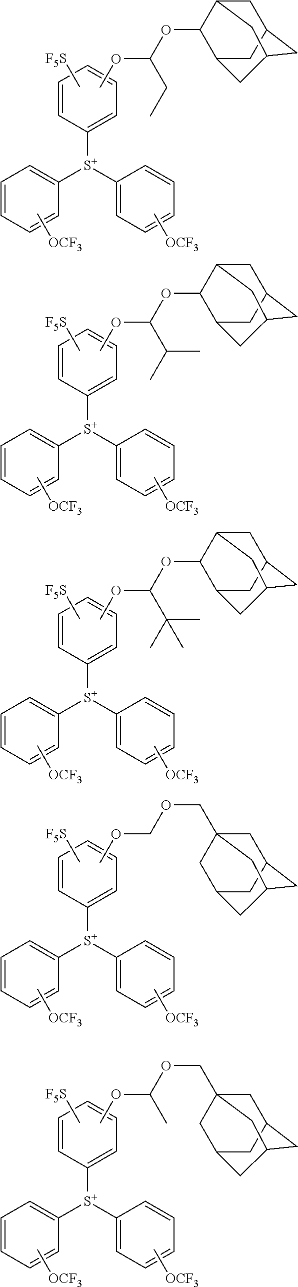

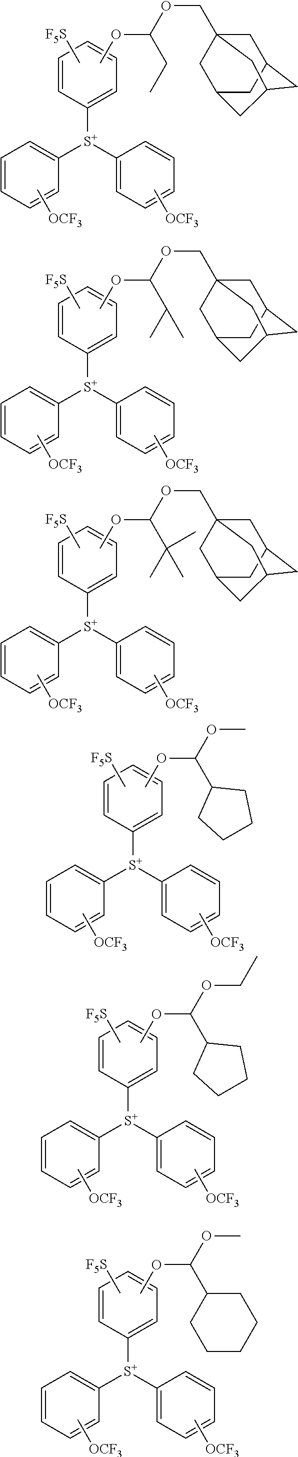

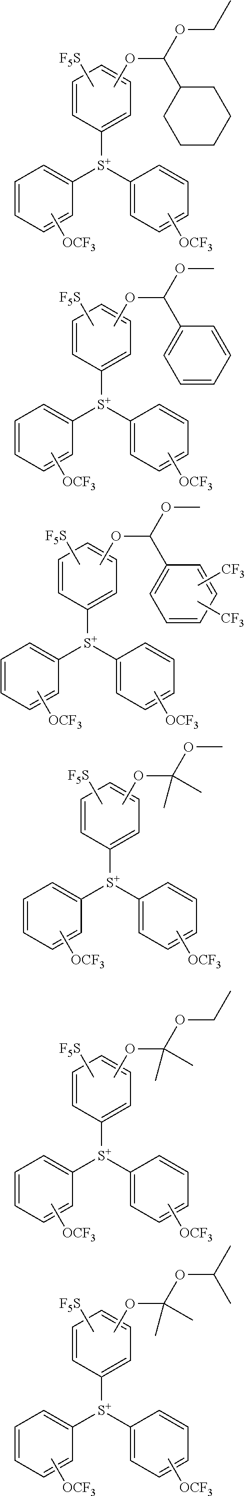

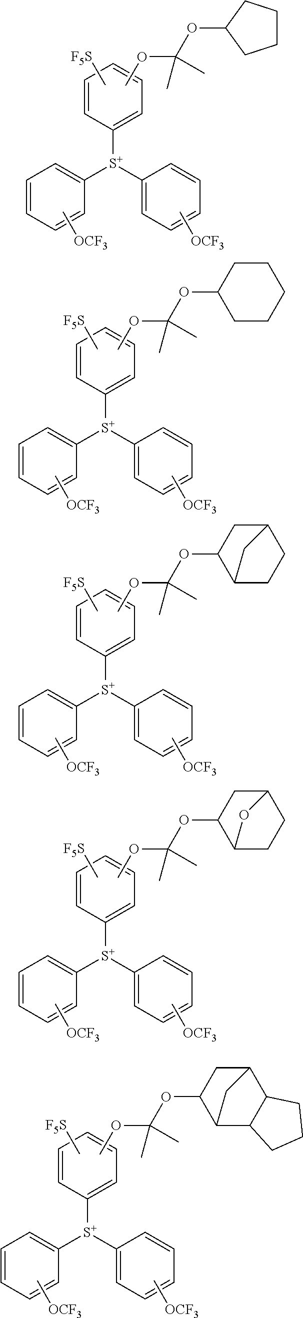

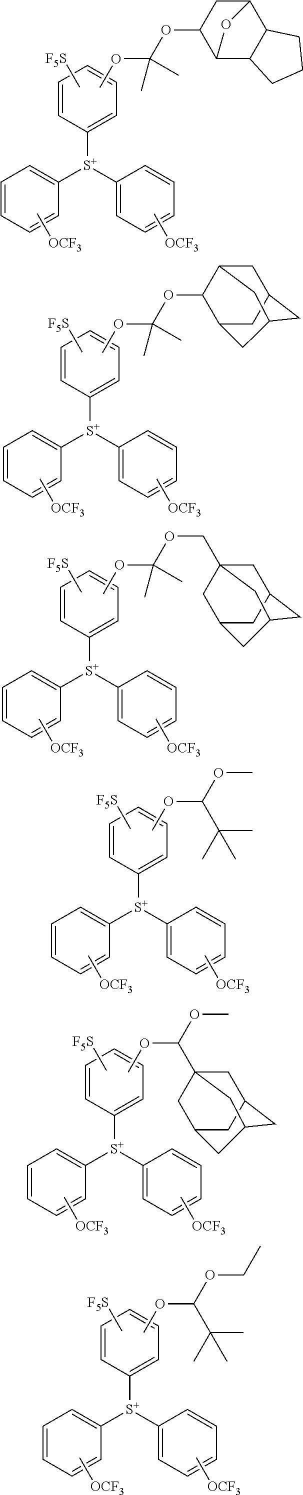

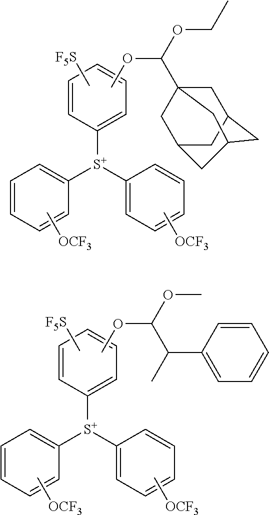

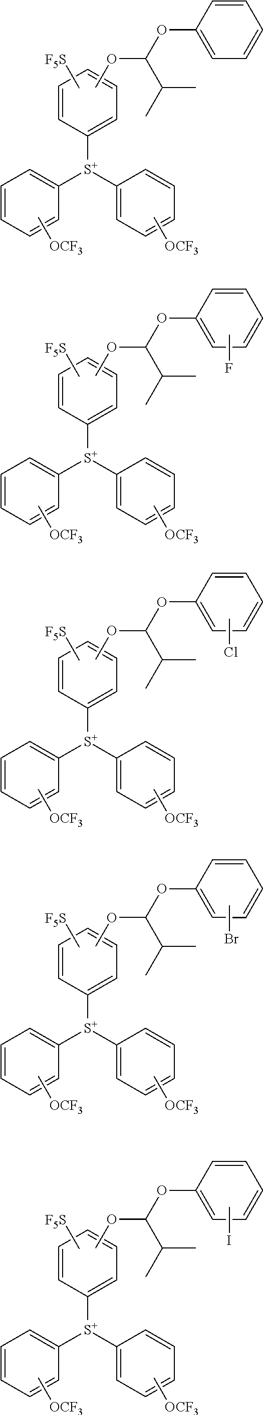

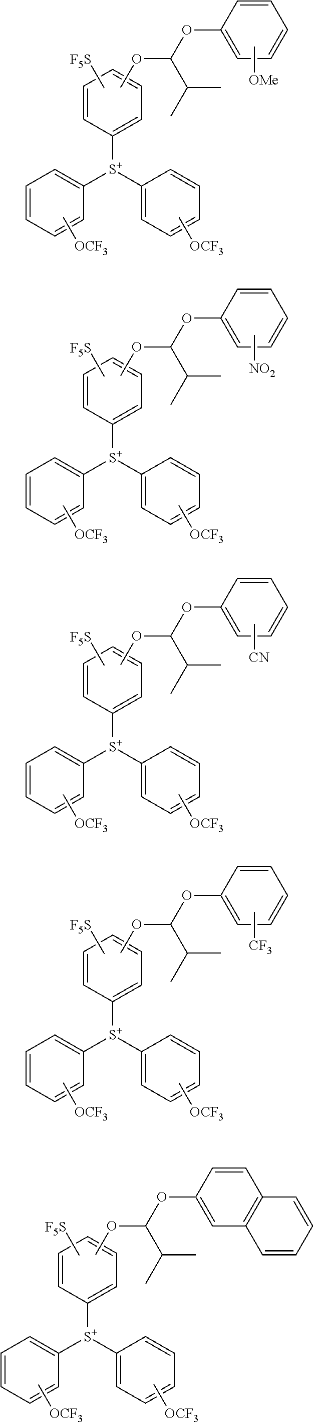

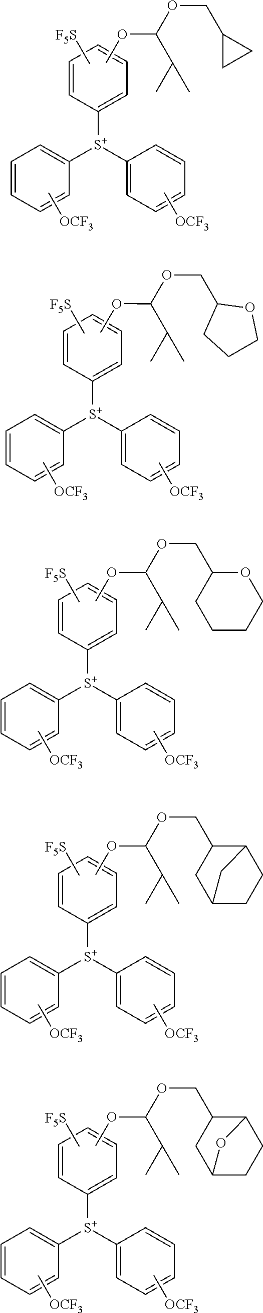

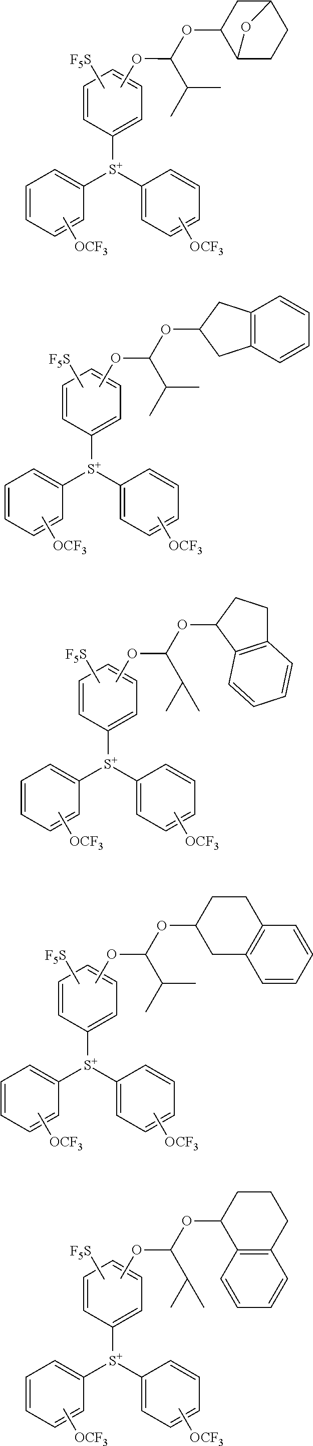

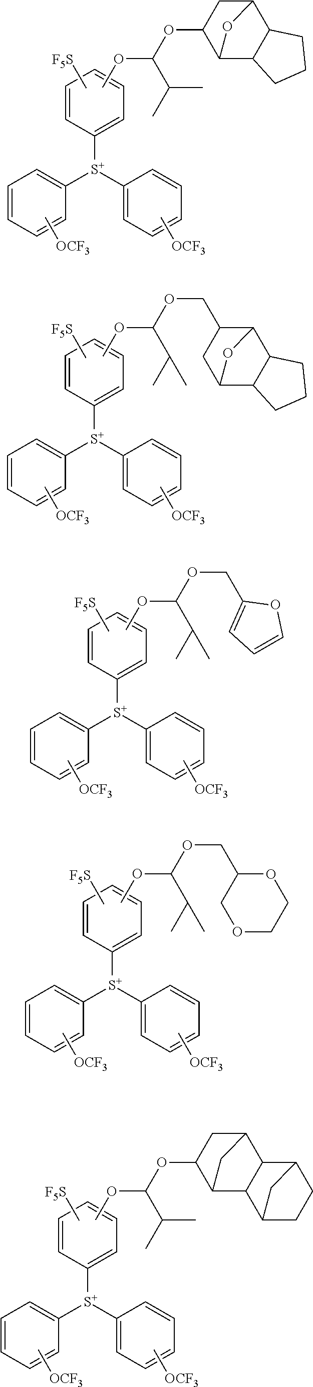

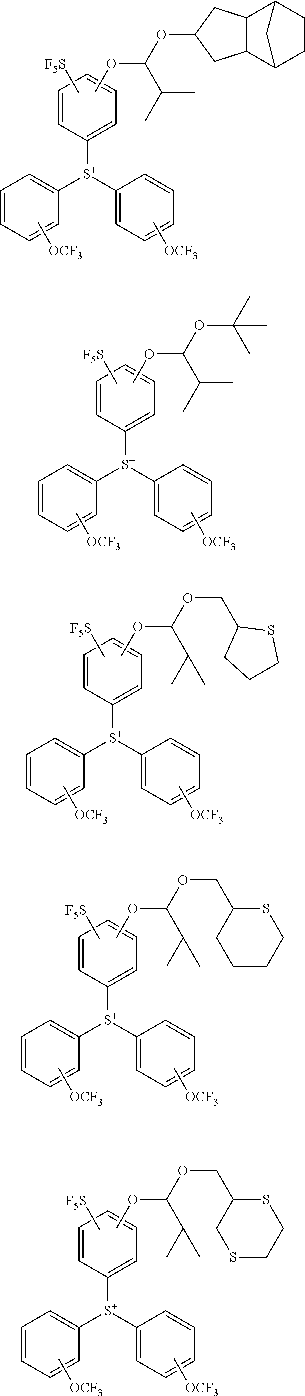

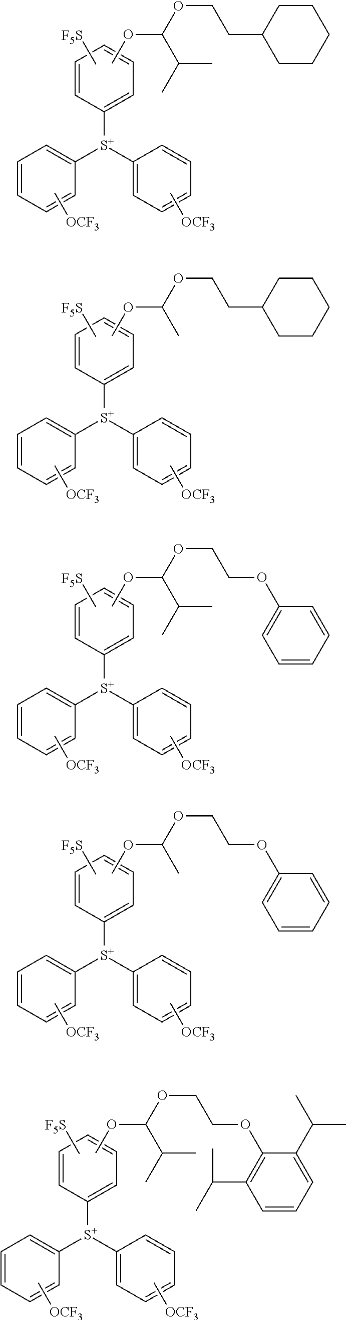

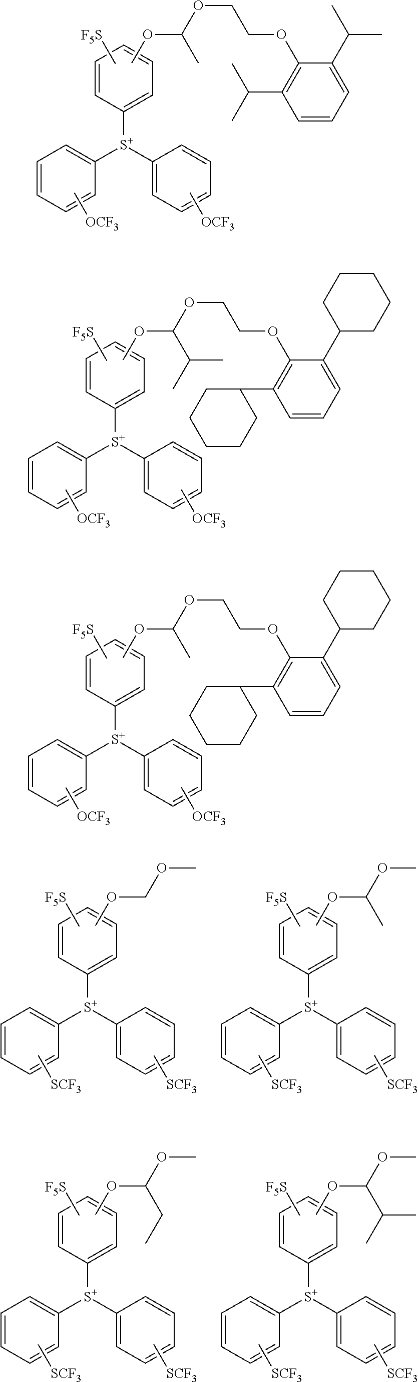

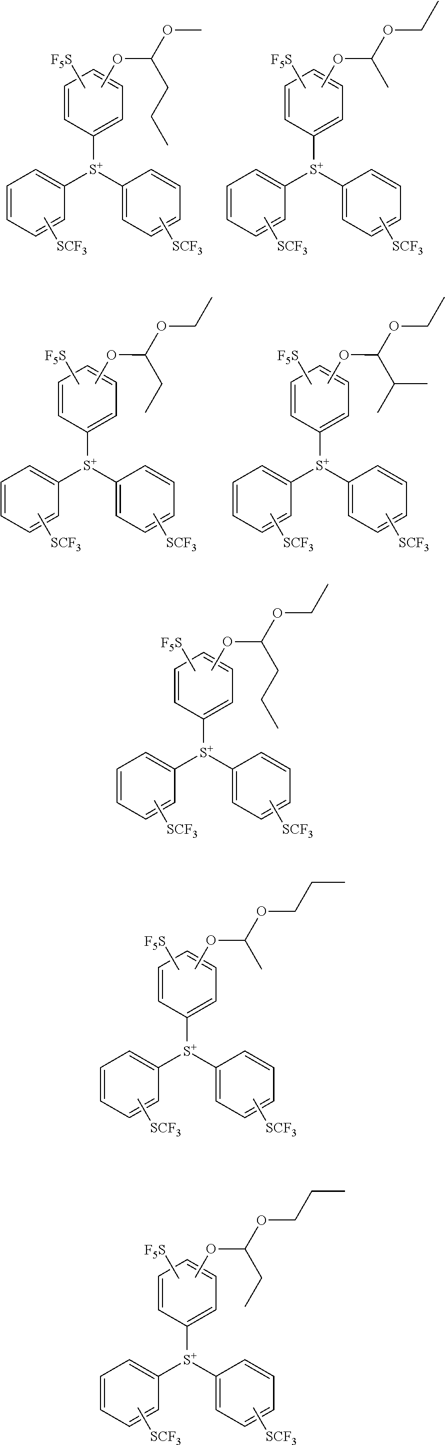

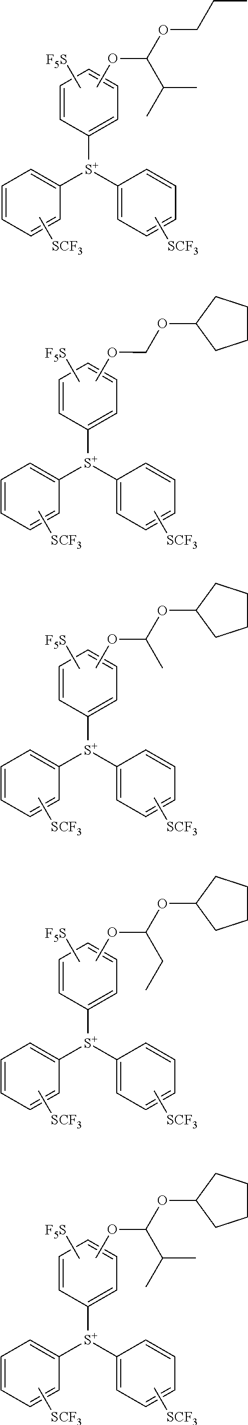

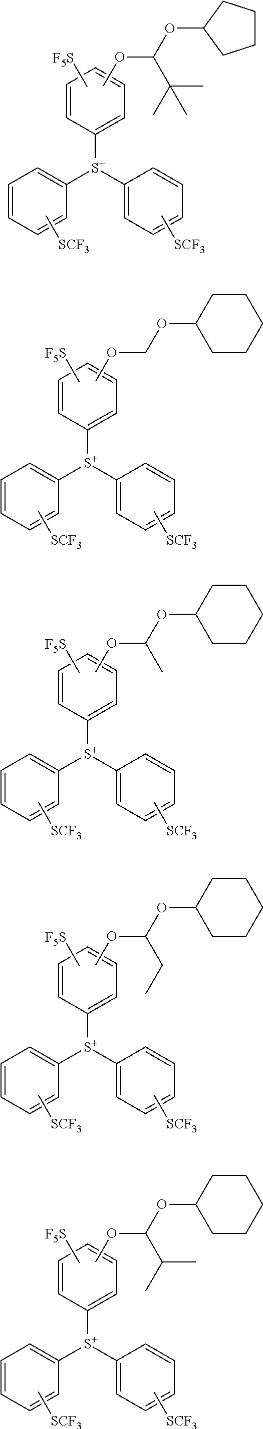

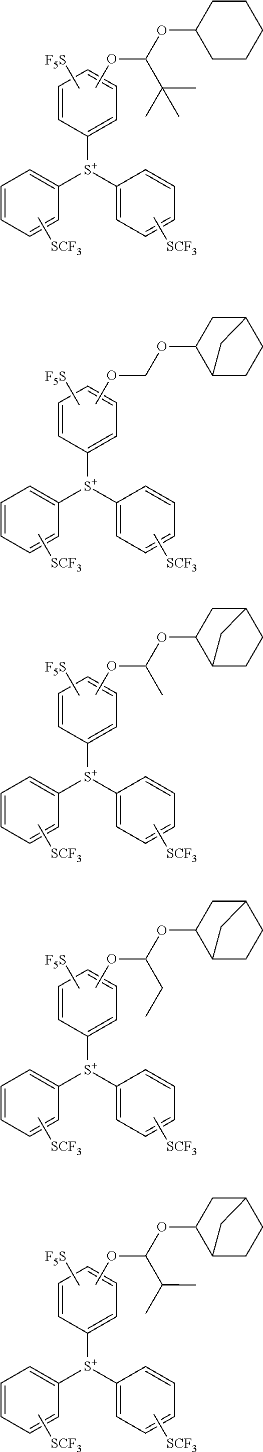

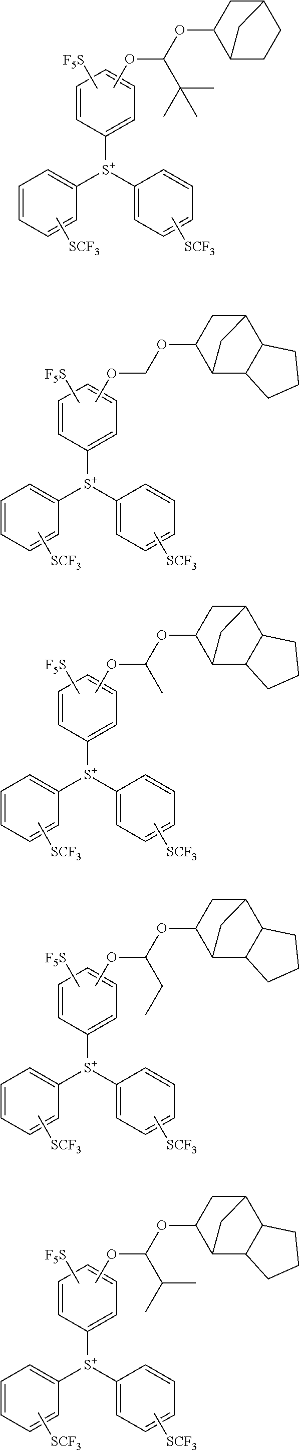

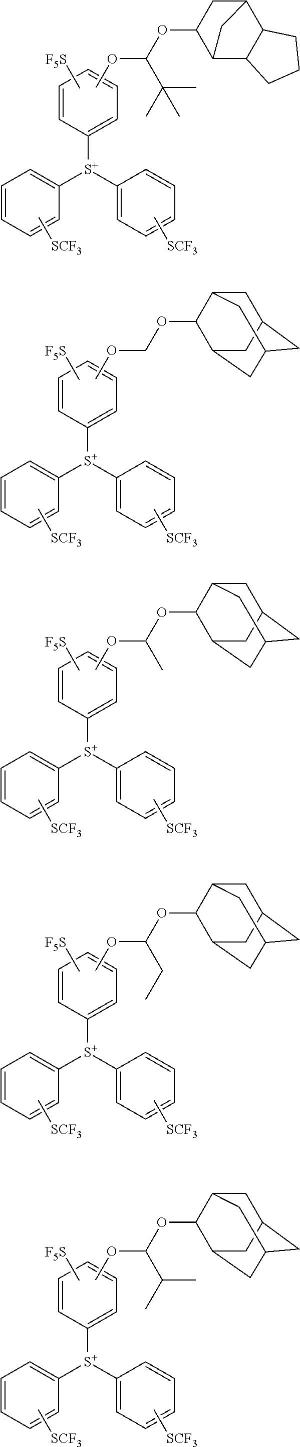

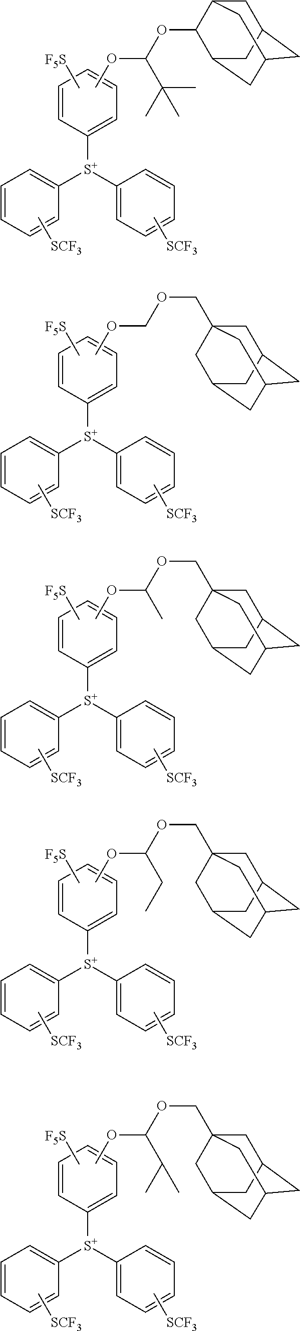

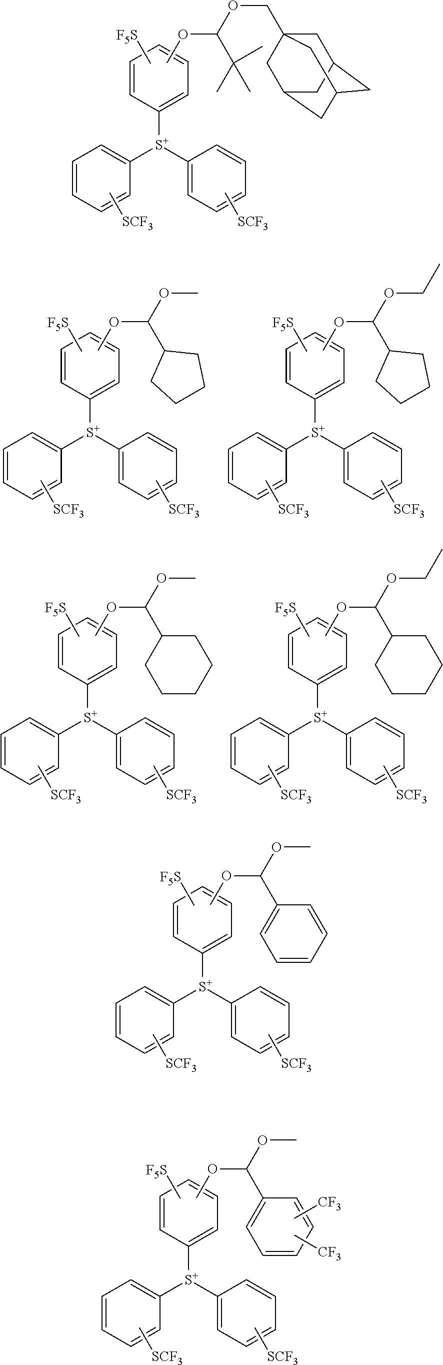

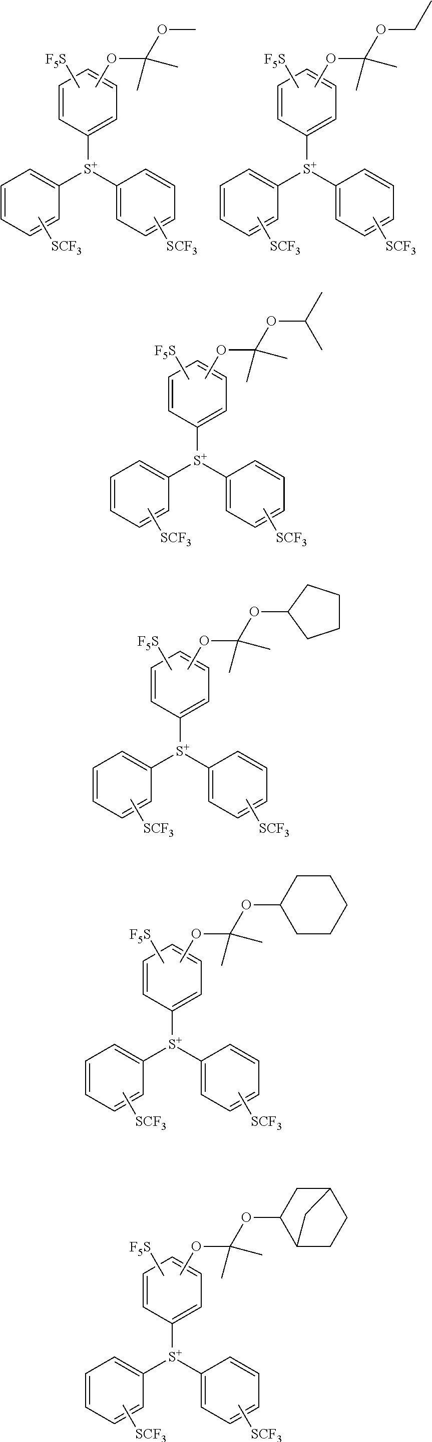

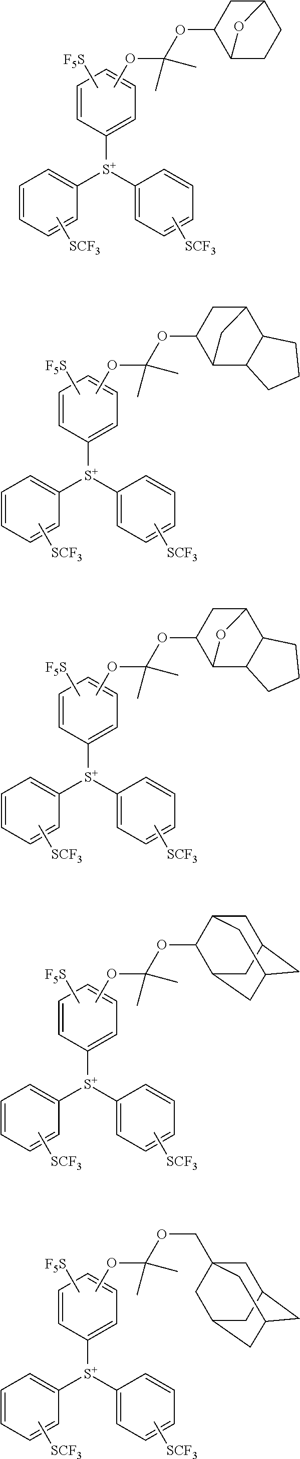

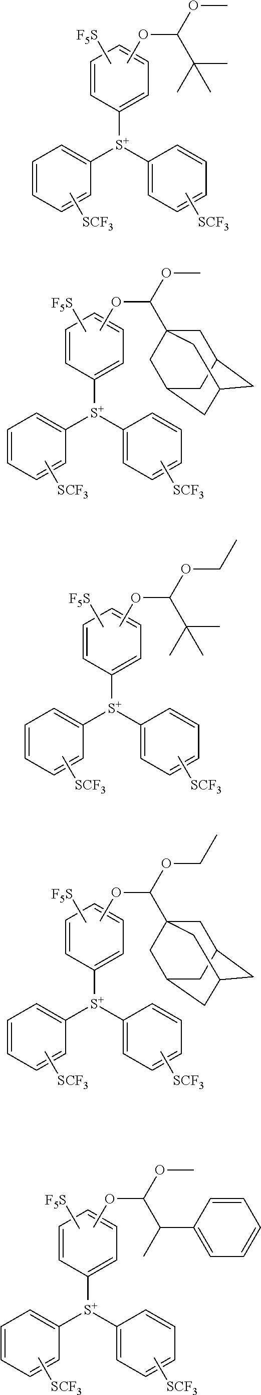

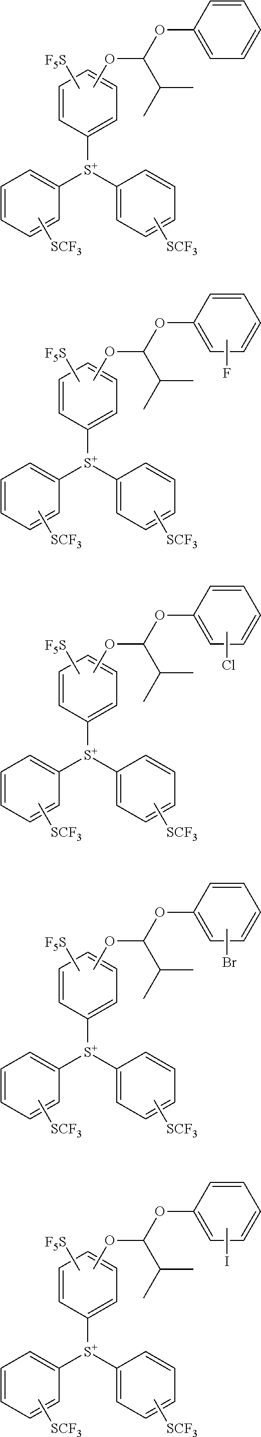

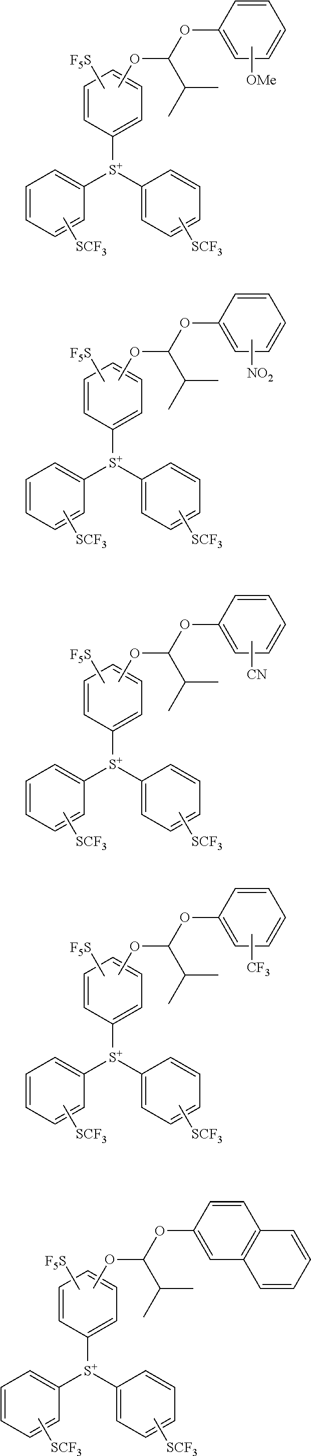

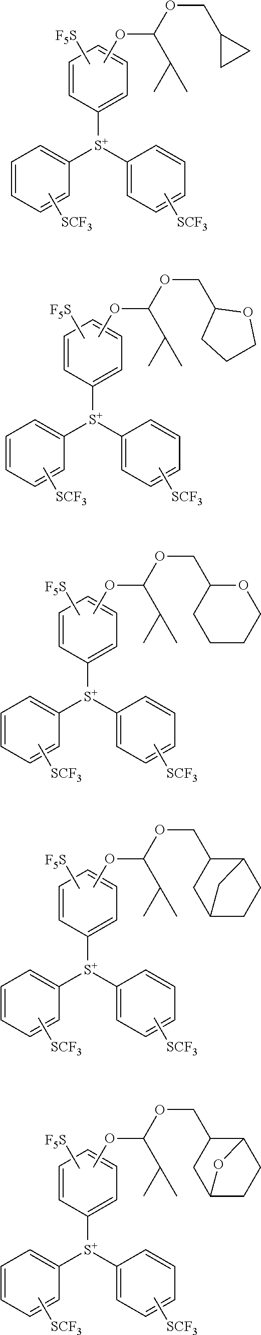

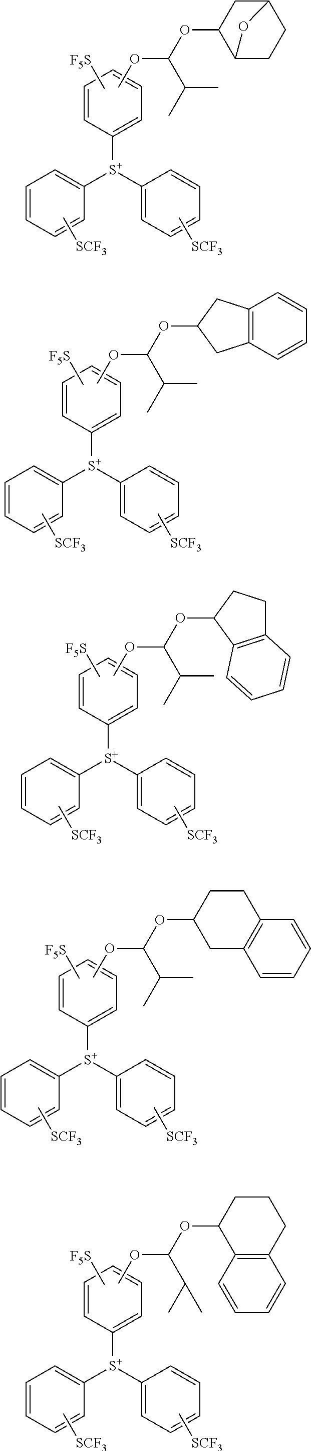

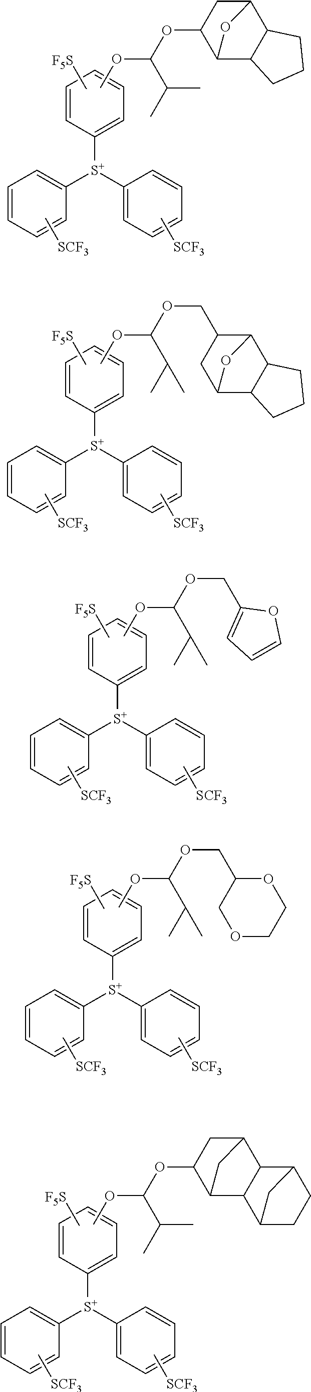

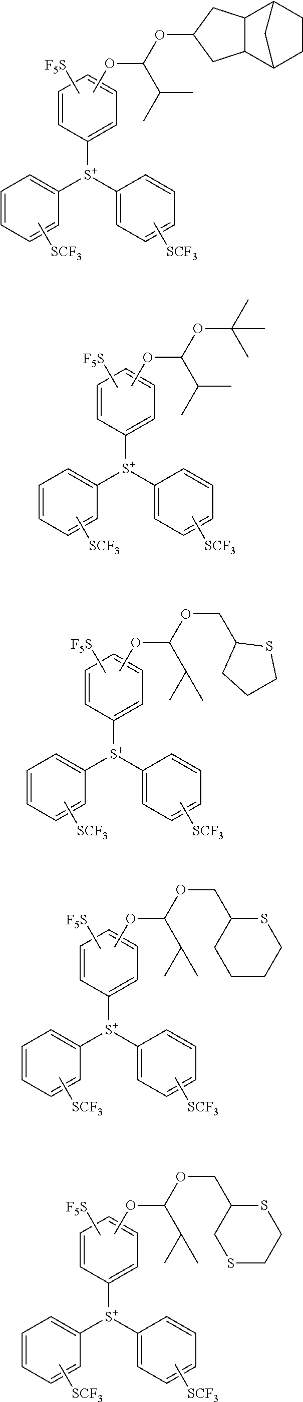

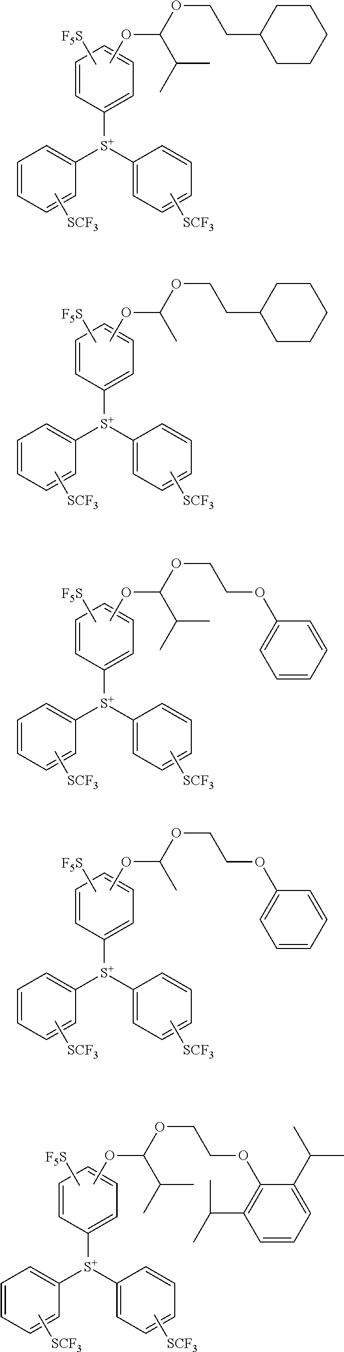

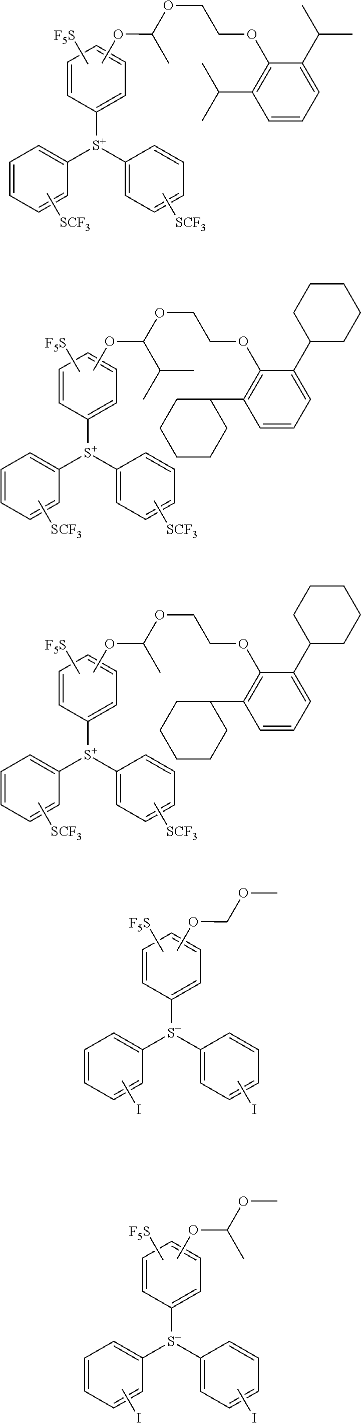

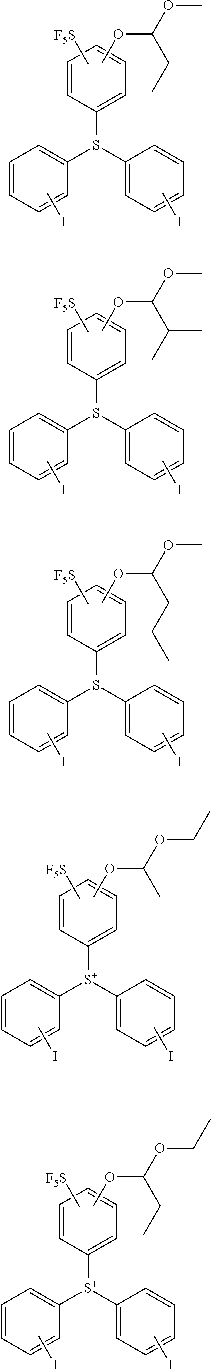

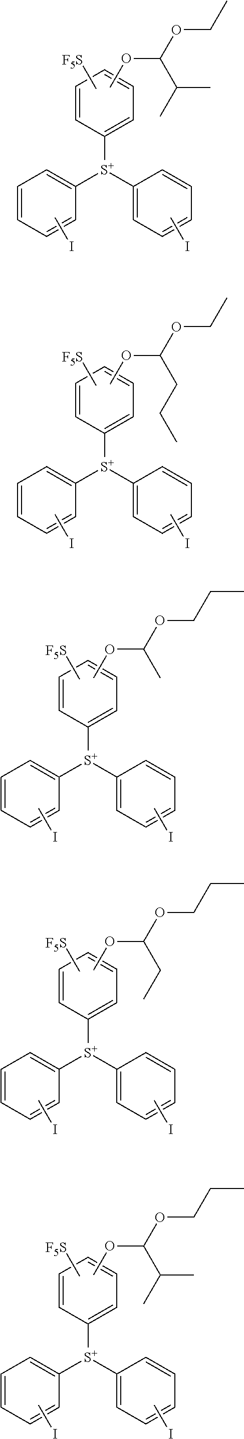

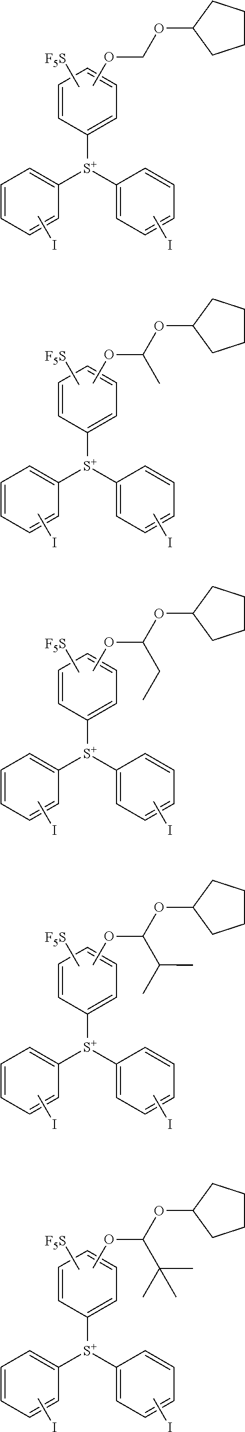

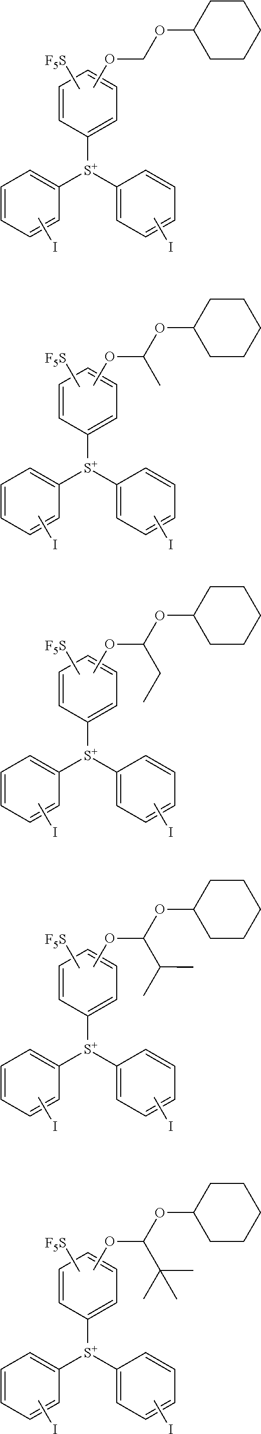

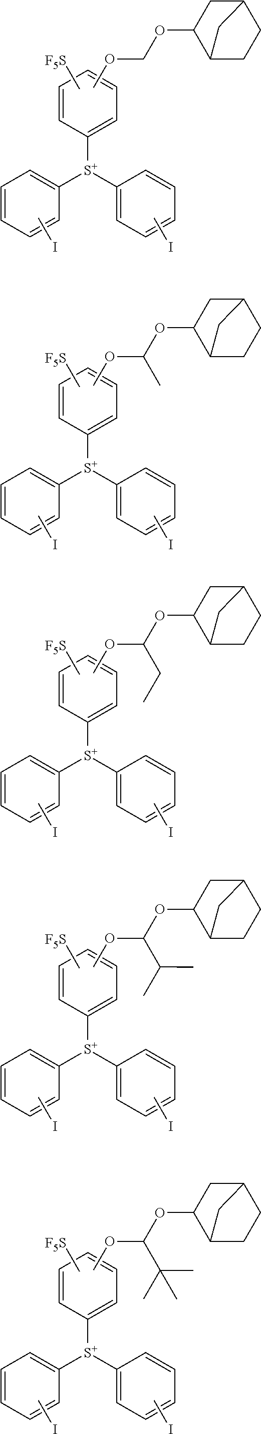

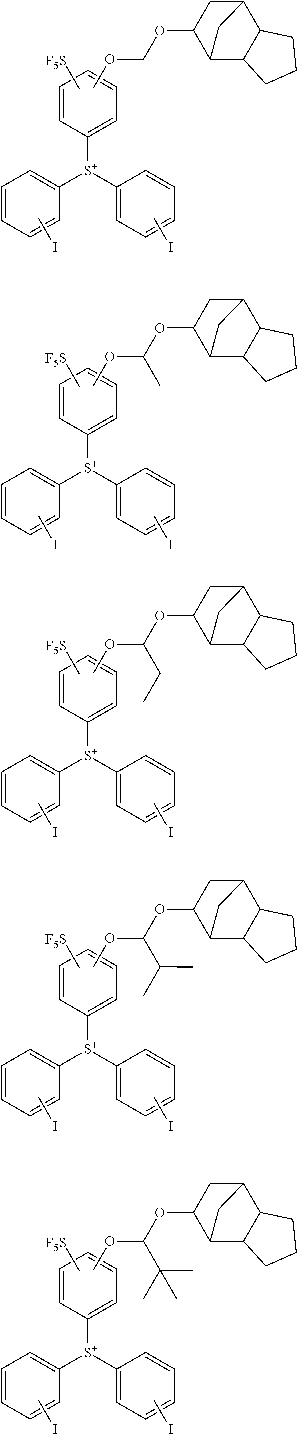

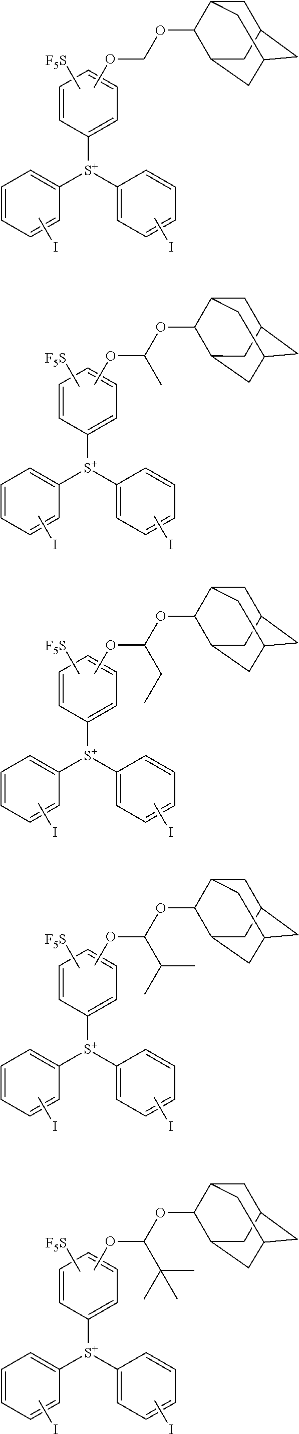

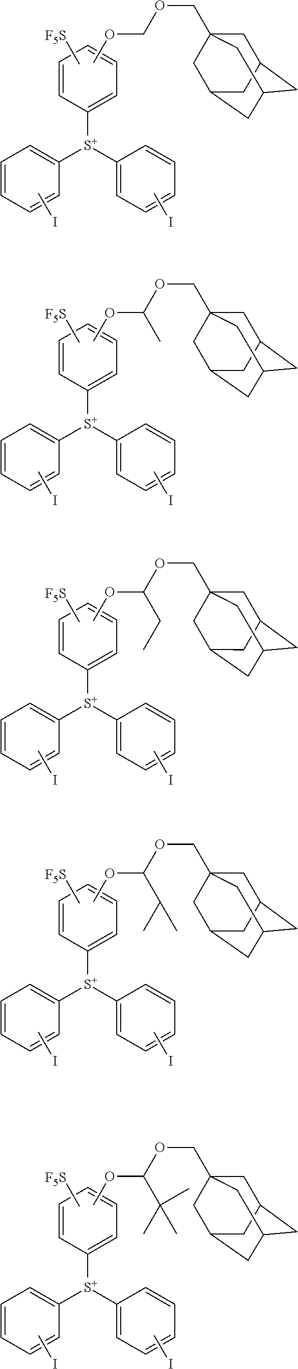

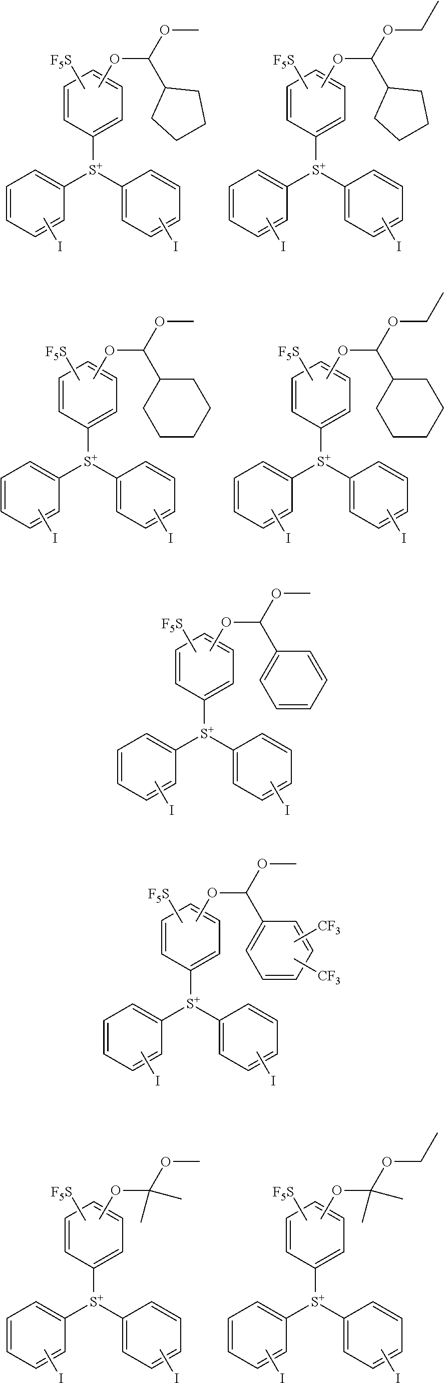

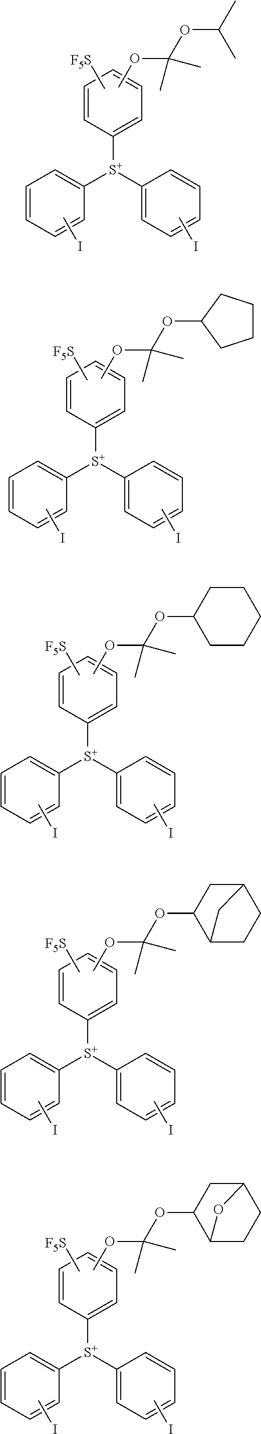

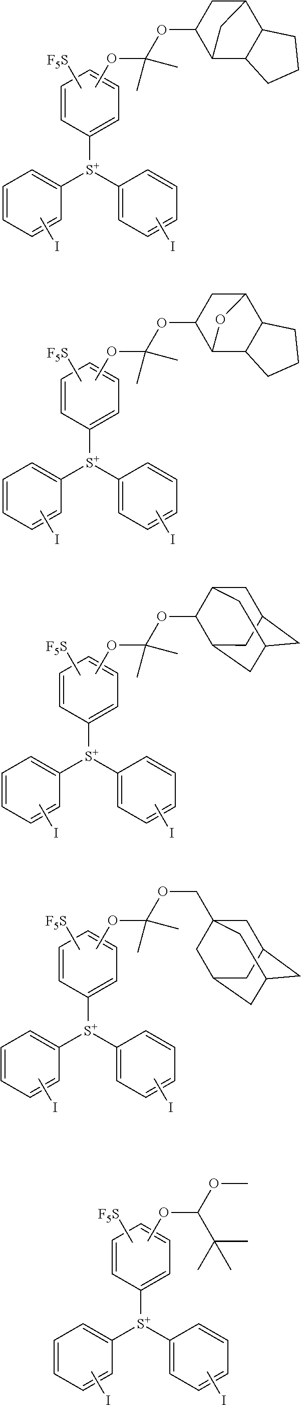

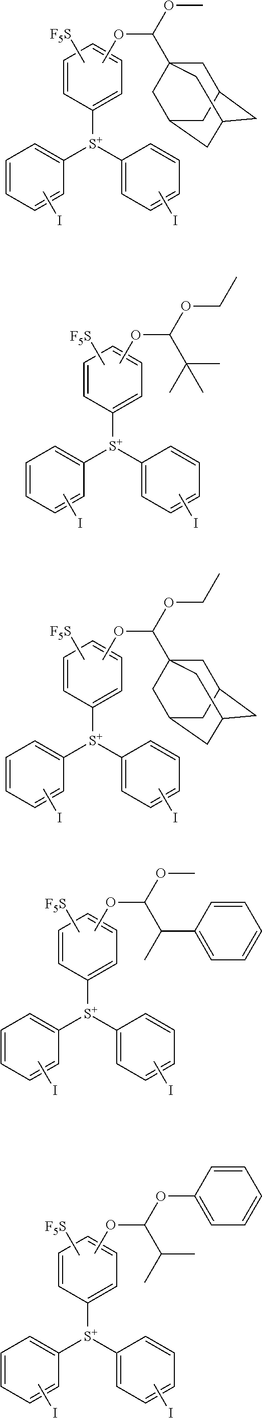

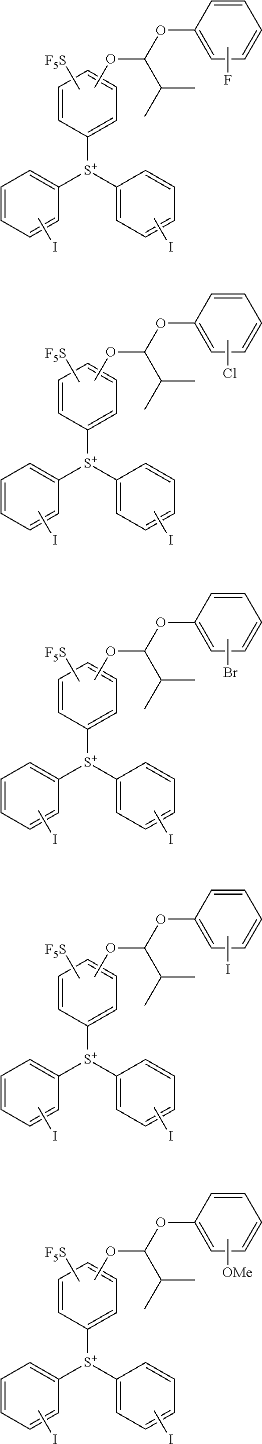

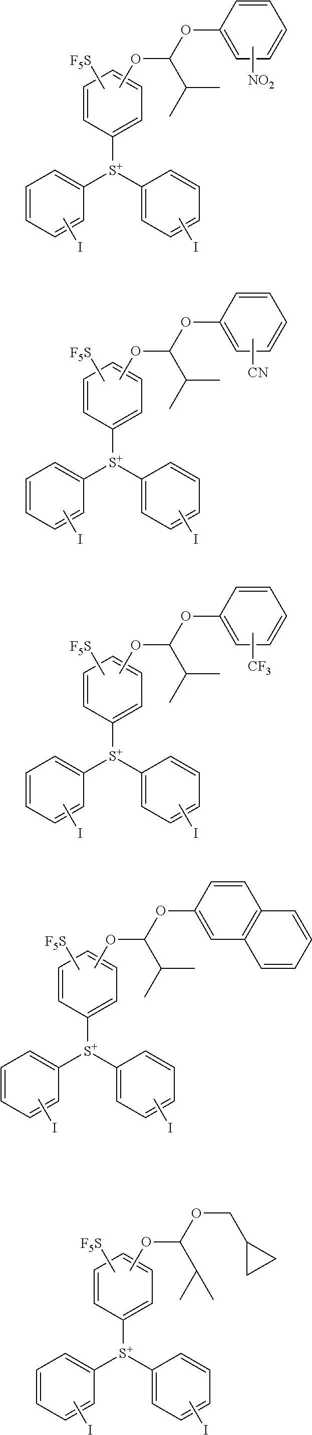

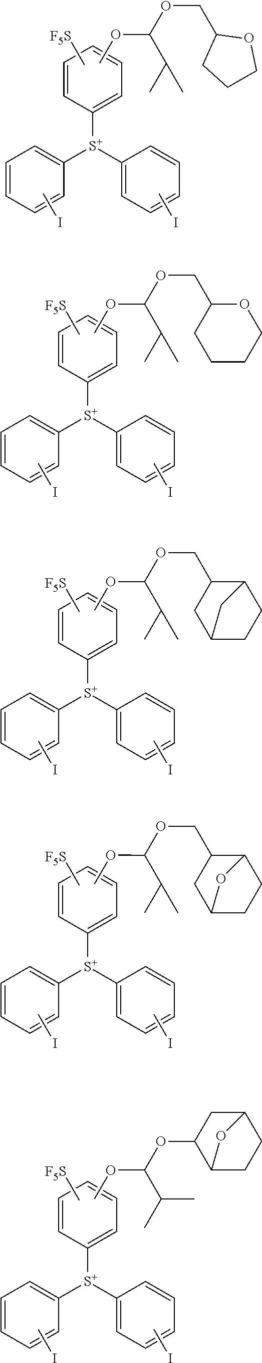

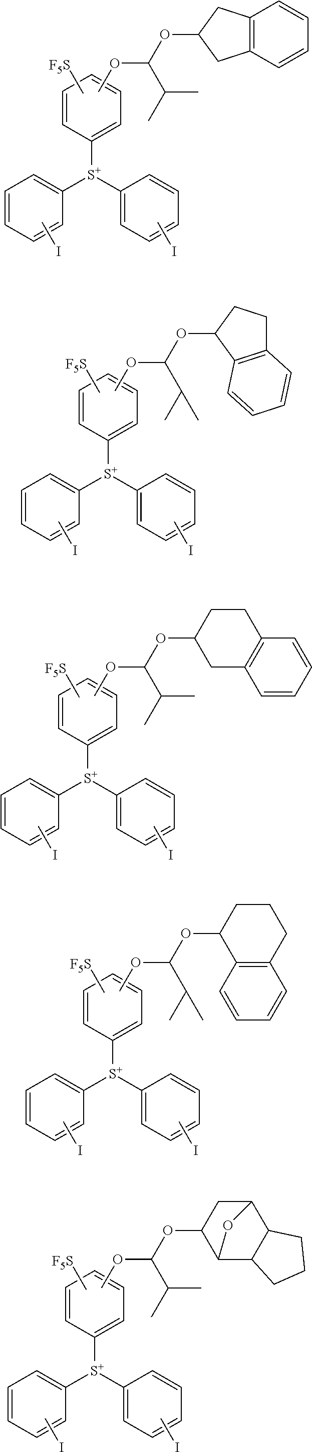

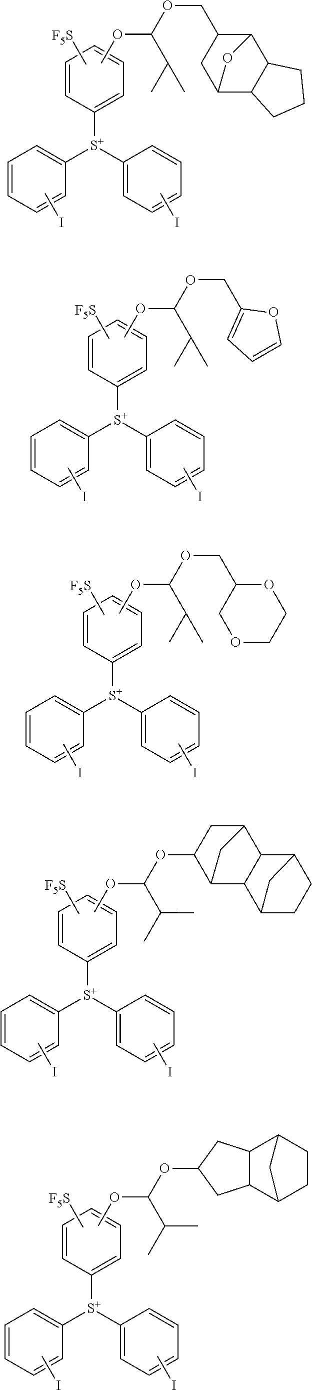

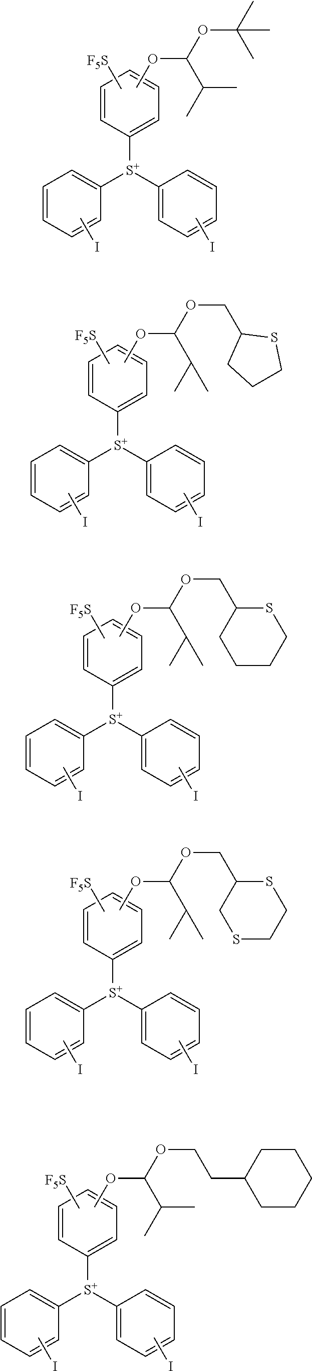

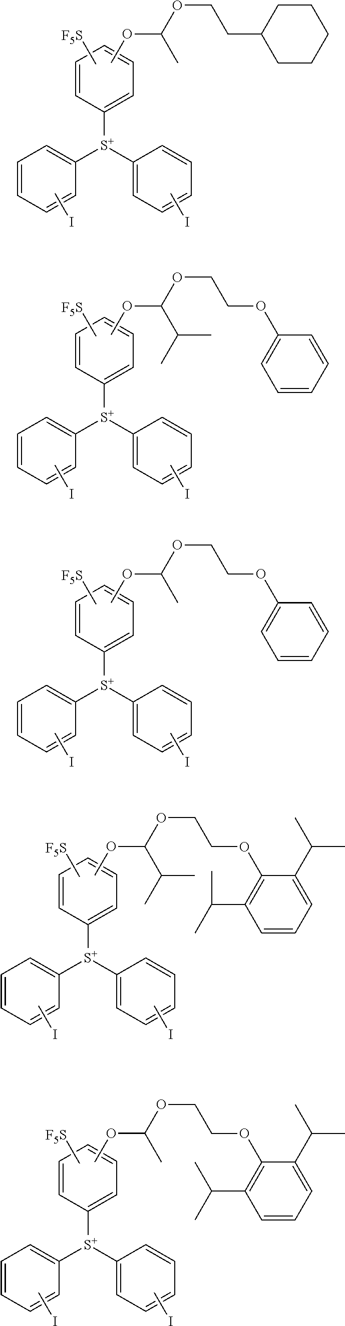

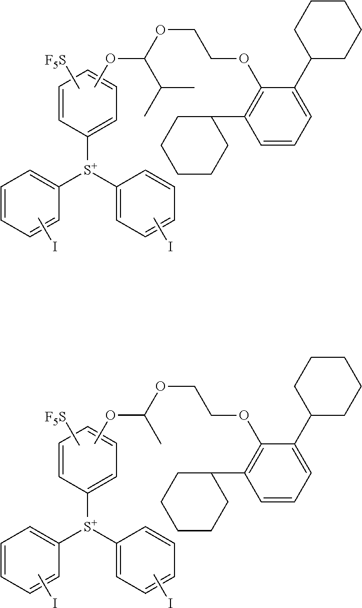

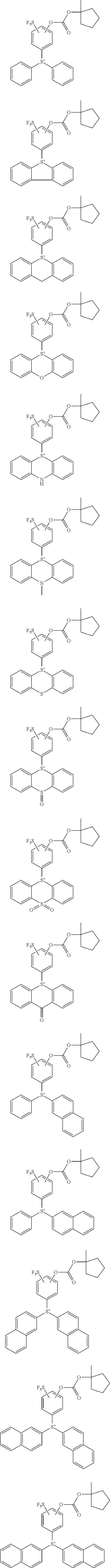

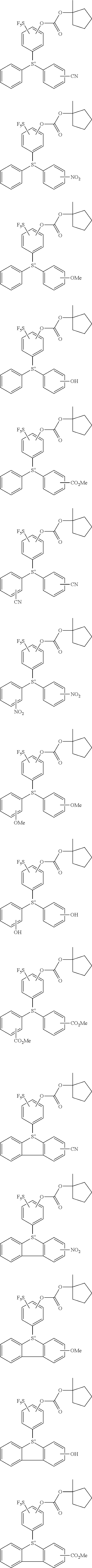

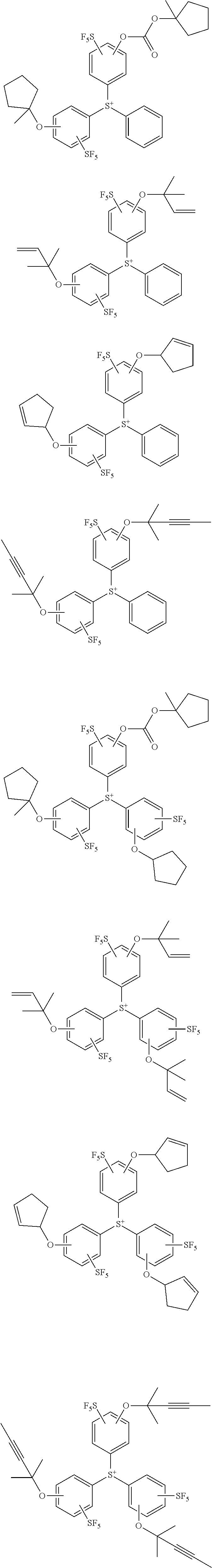

In the formulas (a) and (a1), Z⁻ is a carboxylic acid anion having an aromatic vinyl structure and an iodine atom. The carboxylic acid anion preferably has the following formula (Z):

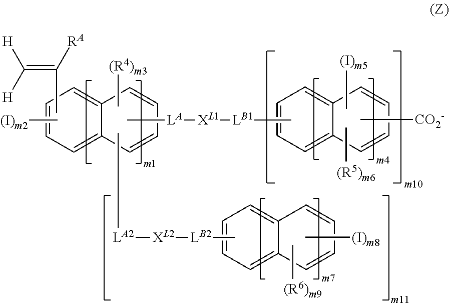

In the formula (Z), m1 is 0 or 1. When m1 is 0, it is a benzene ring, and when m1 is 1, it is a naphthalene ring, but a benzene ring in which m1 is 0 is preferable from a viewpoint of solvent solubility. m2 is 0, 1, 2, 3, or 4. m2 is preferably 0, 1, 2, or 3, more preferably 0, 1, or 2, and still more preferably 0 or 1 from a viewpoint of raw material procurement. m3 is 0, 1, 2, or 3.

In the formula (Z), m4 is 0 or 1. When m4 is 0, it is a benzene ring, and when m4 is 1, it is a naphthalene ring, but a benzene ring in which m4 is 0 is preferable from a viewpoint of solvent solubility. m5 is 0, 1, 2, 3, or 4. m5 is preferably 0, 1, 2, or 3, and more preferably 0, 1, or 2 from a viewpoint of raw material procurement. m6 is 0, 1, 2, or 3.

In the formula (Z), m7 is 0 or 1. When m7 is 0, it is a benzene ring, and when m7 is 1, it is a naphthalene ring, but a benzene ring in which m7 is 0 is preferable from a viewpoint of solvent solubility. m8 is 1, 2, 3, or 4. m8 is preferably 1, 2, or 3, and more preferably 1 or 2 from a viewpoint of raw material procurement. m9 is 0, 1, 2, or 3.

In the formula (Z), m10 is 0 or 1. m11 is 0 or 1.

Provided that 0≤m2+m3+m11≤4 when m1 is 0, and 0≤m2+m3+m11≤6 when m1 is 1. 0≤m5+m6≤4 when m4 is 0, and 0≤m5+m6≤6 when m4 is 1. 0≤m8+m9≤5 when m7 is 0, and 0≤m8+m9≤7 when m7 is 1. The larger the number of iodine atoms in the anion, the higher the absorption particularly for EUV, but since there is a concern that solvent solubility is poor and precipitation occurs in a resist composition, 1≤m2+m5+m8≤4 is preferable.

In the formula (Z), R^{A} is a hydrogen atom, a fluorine atom, a methyl group, or a trifluoromethyl group. Among these, R^{A} is preferably a hydrogen atom or a methyl group, and more preferably a hydrogen atom.

In the formula (Z), R⁴ is a halogen atom other than an iodine atom, a nitro group, a cyano group, a hydroxy group, a carboxy group, a pentafluorosulfanyl group, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₁-C₂₀ hydrocarbyloxycarbonyl group which may contain a heteroatom. The halogen atom other than an iodine atom is preferably a fluorine atom, a chlorine atom, or a bromine atom, and more preferably a fluorine atom. The hydrocarbyl group and hydrocarbyl moieties of the hydrocarbyloxy group and the hydrocarbylthio group may be saturated or unsaturated, and may be linear, branched, or cyclic. Specific examples thereof include: a C₁-C₂₀ alkyl group such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, or an icosyl group; a C₃-C₂₀ cyclic saturated hydrocarbyl group such as a cyclopropyl group, a cyclopentyl group, a cyclohexyl group, a cyclopropylmethyl group, a 4-methylcyclohexyl group, a cyclohexylmethyl group, a norbornyl group, or an adamantyl group; a C₂-C₂₀ alkenyl group such as a vinyl group, a 1-propenyl group, a 2-propenyl group, a butenyl group, or a hexenyl group; a C₃-C₂₀ cyclic unsaturated hydrocarbyl group such as a cyclohexenyl group; a C₆-C₂₀ aryl group such as a phenyl group or a naphthyl group; a C₇-C₂₀ aralkyl group such as a benzyl group, a 1-phenylethyl group, or a 2-phenylethyl group; and a group obtained by combining these groups. Some or all of hydrogen atoms of the hydrocarbyl group may be replaced with a group containing a heteroatom such as an oxygen atom, a sulfur atom, a nitrogen atom, or a halogen atom, some of (—CH₂—)s in the hydrocarbyl group may be replaced with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic acid anhydride (—C(—O)—O—C(—O)—), a haloalkyl group, or the like. When m3 is 2 or 3, R⁴s may be the same as or different from each other.

In addition, when m3 is 2 or 3, two R⁴s may be bonded to each other to form a ring together with aromatic ring carbon atoms to which they are bonded. Specific examples of the ring formed at this time include a cyclopropane ring, a cyclobutane ring, a cyclopentane ring, a cyclohexane ring, a norbornane ring, and an adamantane ring. Some or all of hydrogen atoms of the ring may be replaced with a group containing a heteroatom such as an oxygen atom, a sulfur atom, a nitrogen atom, or a halogen atom, some of (—CH₂—)s in the ring may be replaced with a group containing a heteroatom such as an oxygen atom, a sulfur atom, or a nitrogen atom, and as a result, the ring may contain a hydroxy group, a fluorine atom, a chlorine atom, a bromine atom, an iodine atom, a cyano group, a carbonyl group, an ether bond, an ester bond, a sulfonate bond, a carbonate bond, a lactone ring, a sultone ring, a carboxylic acid anhydride (—C(═O)—O—C(—O)—), a haloalkyl group, or the like.

In the formula (Z), R⁵ is a halogen atom other than an iodine atom, a nitro group, a cyano group, a hydroxy group, a carboxy group, a pentafluorosulfanyl group, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₁-C₂₀ hydrocarbyloxycarbonyl group which may contain a heteroatom. Specific examples of the halogen atom other than an iodine atom include a fluorine atom, a chlorine atom, a bromine atom. The hydrocarbyl group and hydrocarbyl moieties of the hydrocarbyloxy group and the hydrocarbylthio group may be saturated or unsaturated, and may be linear, branched, or cyclic. Specific examples thereof include, but are not limited to, those similar to those exemplified as the hydrocarbyl group of R⁴. Some or all of hydrogen atoms of the hydrocarbyl group may be replaced with a group containing a heteroatom such as an oxygen atom, a sulfur atom, a nitrogen atom, or a halogen atom, some of (—CH₂—)s in the hydrocarbyl group may be replaced with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like. When m6 is 2 or 3, R⁵s may be the same as or different from each other.

When m6 is 2, two R⁵s may be bonded to each other to form a ring together with carbon atoms to which they are bonded. The ring is preferably a 5- to 8-membered ring.

In the formula (Z), R⁶ is a halogen atom other than an iodine atom, a nitro group, a cyano group, a hydroxy group, a carboxy group, a pentafluorosulfanyl group, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₁-C₂₀ hydrocarbyloxycarbonyl group which may contain a heteroatom. Specific examples of the halogen atom other than an iodine atom include a fluorine atom, a chlorine atom, a bromine atom. The hydrocarbyl group and hydrocarbyl moieties of the hydrocarbyloxy group and the hydrocarbylthio group may be saturated or unsaturated, and may be linear, branched, or cyclic. Specific examples thereof include, but are not limited to, those similar to those exemplified as the hydrocarbyl group of R⁴. Some or all of hydrogen atoms of the hydrocarbyl group may be replaced with a group containing a heteroatom such as an oxygen atom, a sulfur atom, a nitrogen atom, or a halogen atom, some of (—CH₂—)s in the hydrocarbyl group may be replaced with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic acid anhydride (—C(═O)—O—C(—O)—), a haloalkyl group, or the like. When m9 is 2 or 3, R⁵s may be the same as or different from each other.

When m9 is 2, two R⁶s may be bonded to each other to form a ring together with carbon atoms to which they are bonded. The ring is preferably a 5- to 8-membered ring.

In the formula (Z), L^{A1}, L^{A2}, L^{B1}, and L^{B2} are each independently a single bond, an ether bond, a carbonyl group, an ester bond, a sulfonate bond, an amide bond, a sulfonamide bond, a carbonate bond, or a carbamate bond. Among these, L^{A1} is preferably a single bond, an ether bond, an ester bond, or a sulfonate bond, and more preferably an ether bond, an ester bond, or a sulfonate bond. L^{A2} is preferably a single bond, an ether bond, an ester bond, an amide bond, a sulfonamide bond, or a sulfonate bond, and more preferably an ester bond or a sulfonate bond. L^{B1} is preferably a single bond, an ether bond, an ester bond, an amide bond, or a sulfonate bond, and more preferably a single bond, an ether bond, or an ester bond. L^{B2} is preferably a single bond, an ether bond, an ester bond, an amide bond, or a sulfonate bond, and more preferably a single bond, an ether bond, or an ester bond.

When m11 is 1, L^{A1} and L^{A2} are preferably bonded to adjacent carbon atoms of the aromatic ring. At this time, since the substituent including a fluorosulfonic acid anion structure and the substituent including an aromatic ring substituted with an iodine atom are present at spatially closer positions, higher sensitivity is expected.

In the formula (Z), X^{L1} and X¹² are each independently a single bond or a C₁-C₄₀ hydrocarbylene group which may contain a heteroatom. The hydrocarbylene group may be linear, branched, or cyclic, and specific examples thereof include an alkanediyl group, a cyclic saturated hydrocarbylene group, and an arylene group. Specific examples of the heteroatom include an oxygen atom, a nitrogen atom, and a sulfur atom.

Specific examples of the C₁-C₄₀ hydrocarbylene group of X^{L1} and X^{L2} which may contain a heteroatom include, but are not limited to, those listed below. Note that, in the following formulas, * is a bond with L^{A1} and L^{B1}, or a bond with L^{A2} and L^{B2}.

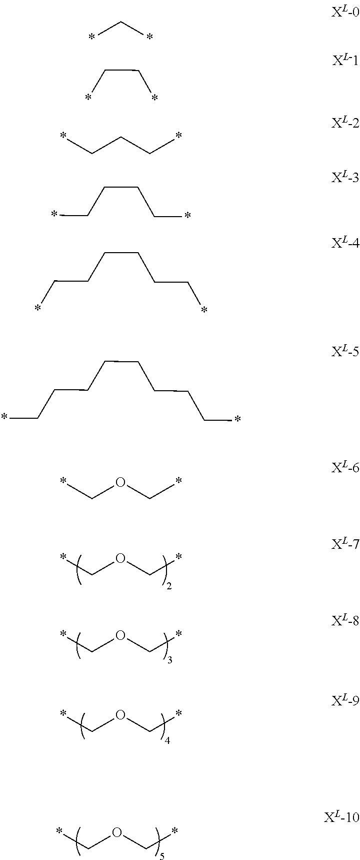

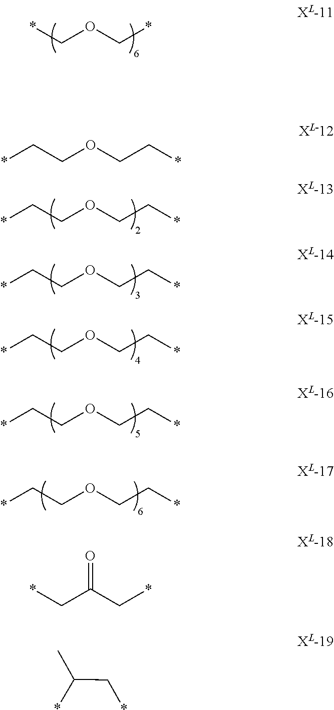

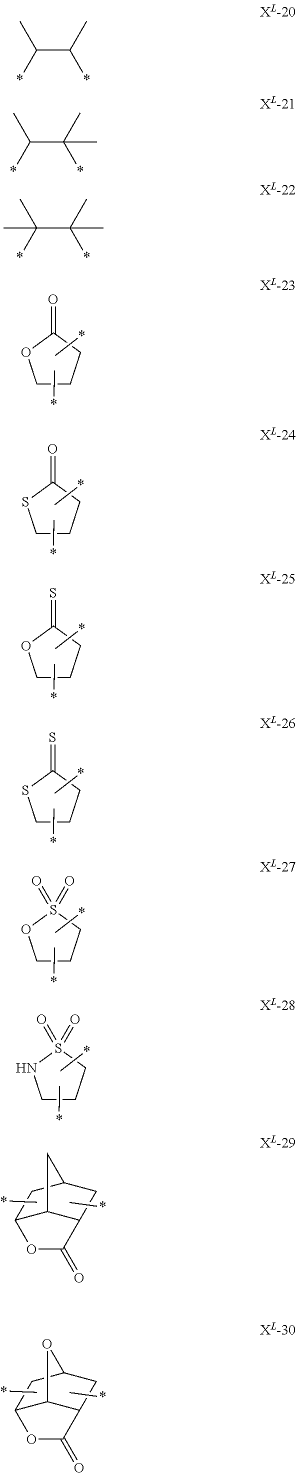

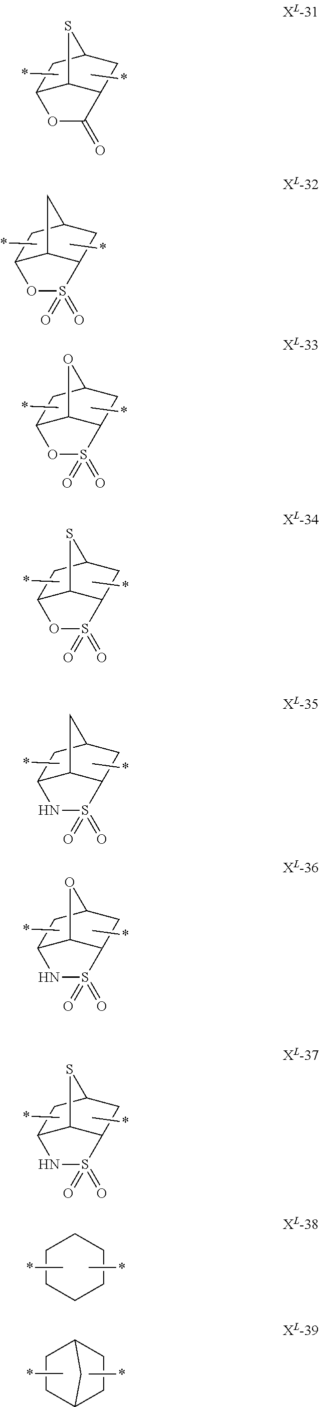

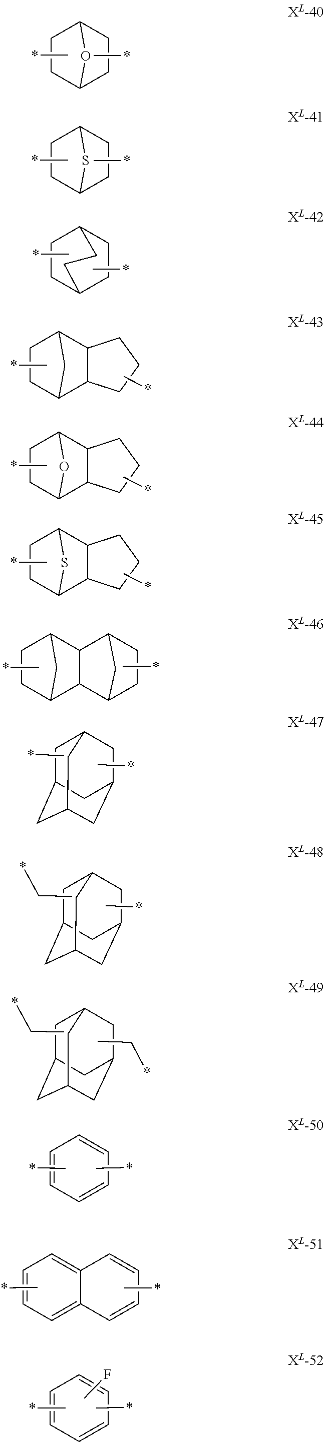

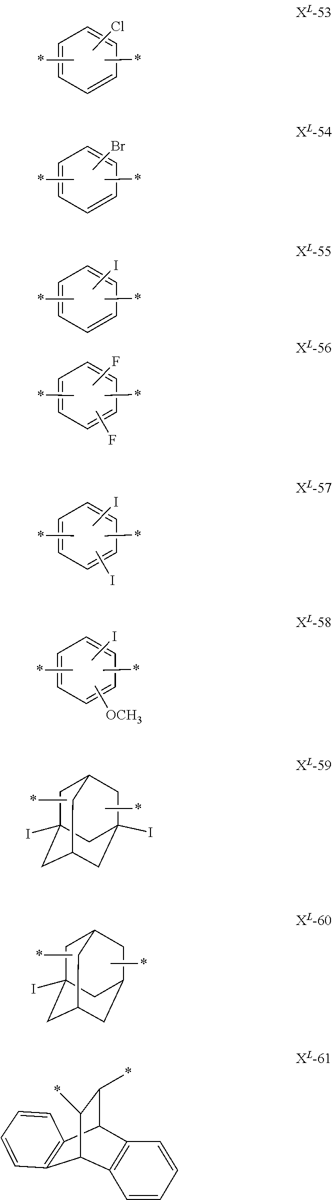

Among these, X^{L}-0 to X^{L}-22, X^{L}-29 to X^{L}-34, and X^{L}-47 to X¹-61 are preferable.

Specific examples of the anion of the onium salt monomer having the formula (a) include, but are not limited to, those listed below. Note that, in the following formulas, R^{A} is as defined above, and Me is a methyl group. Bonding positions of various substituents on an aromatic ring may be replaced with each other on the aromatic ring.

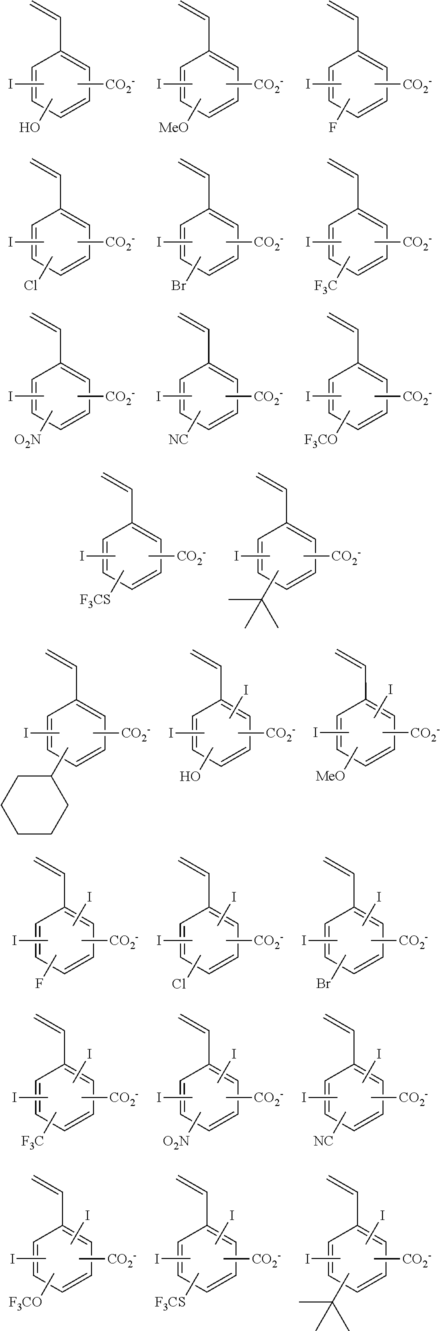

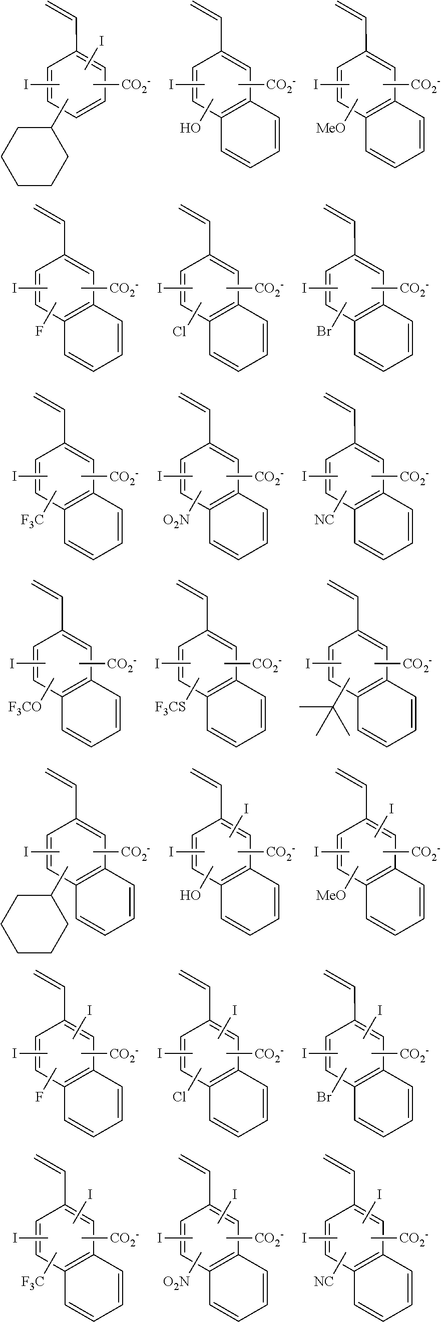

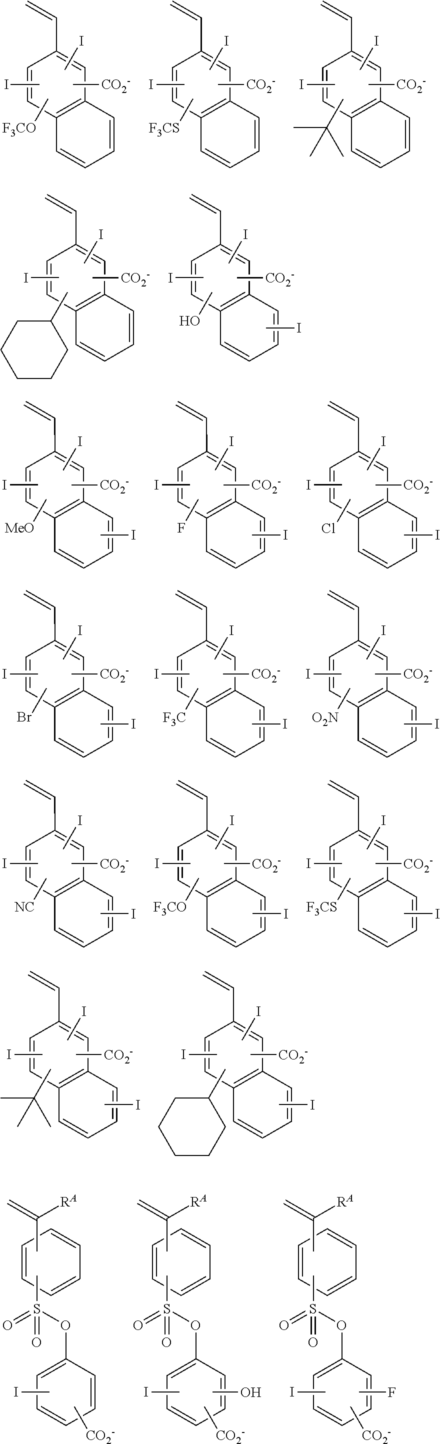

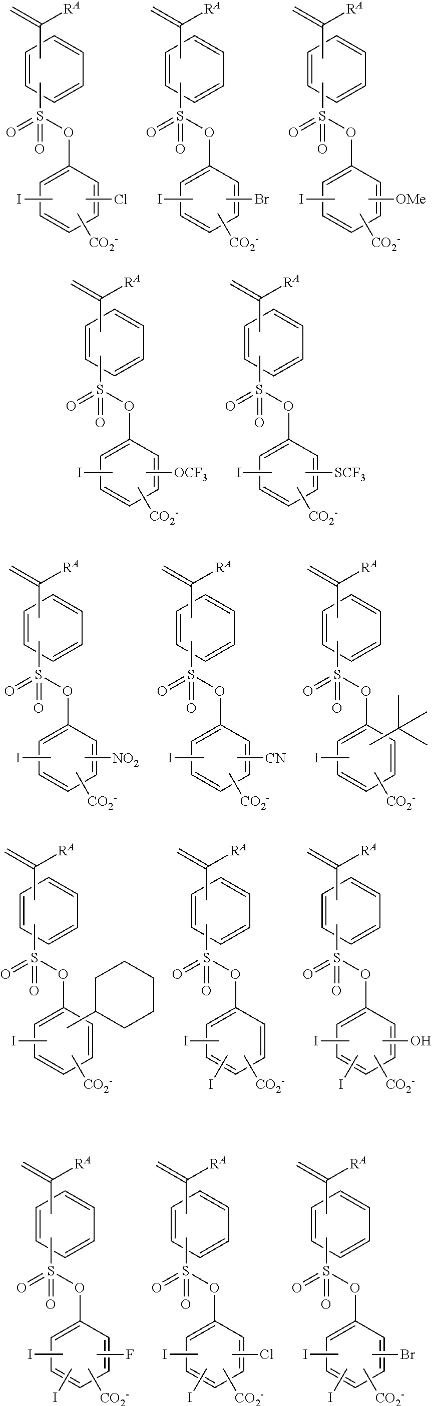

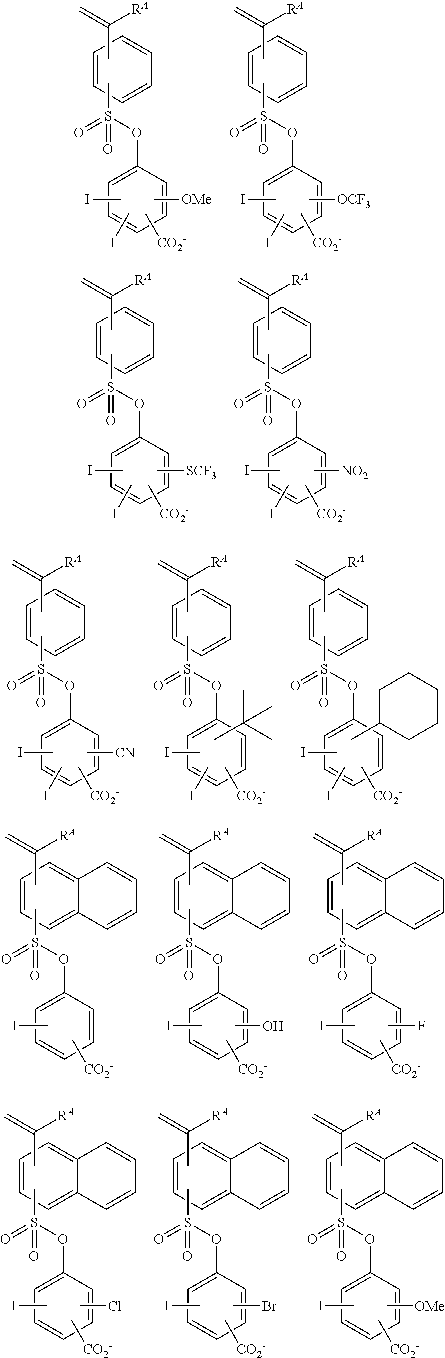

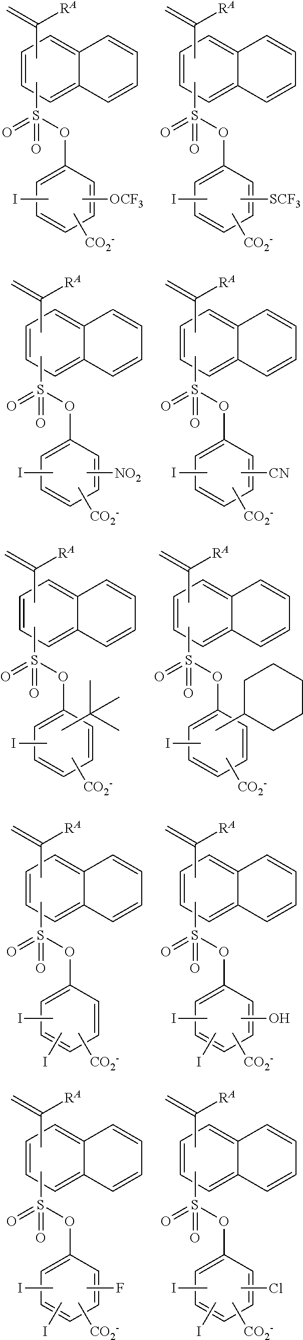

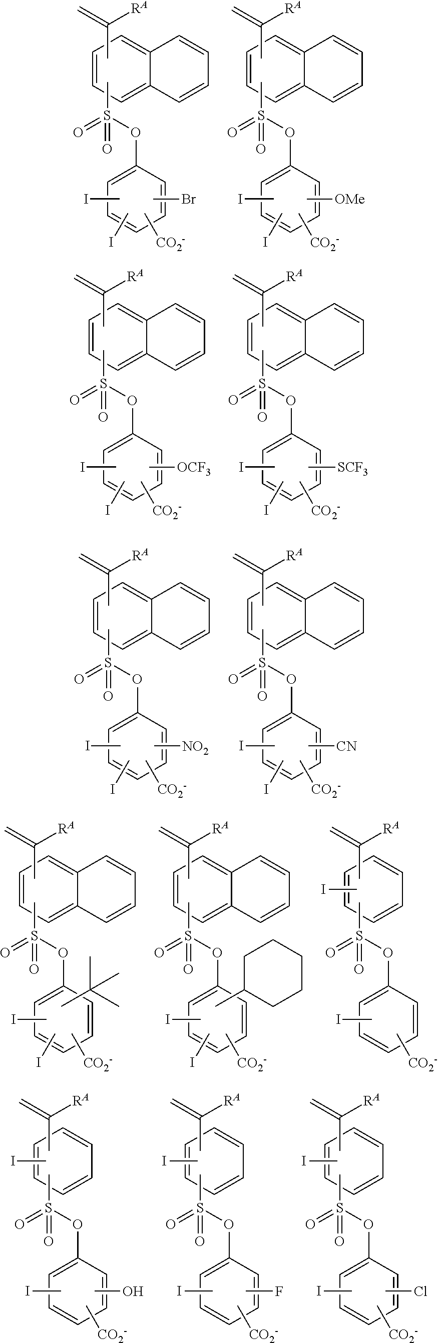

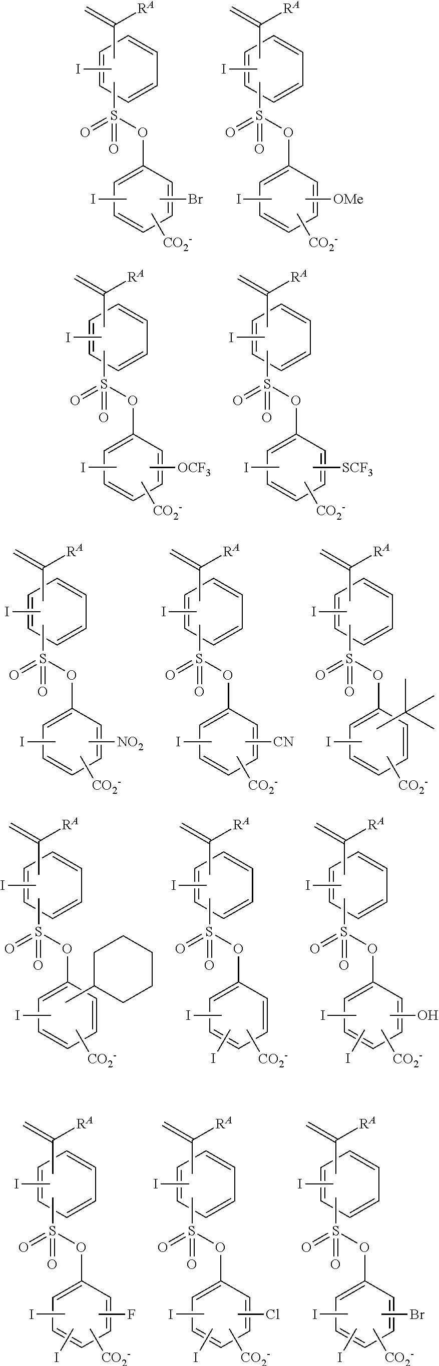

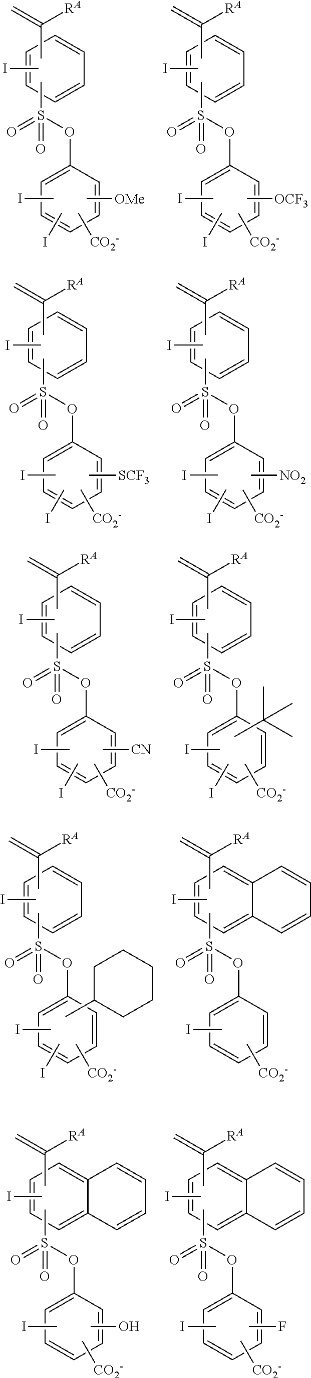

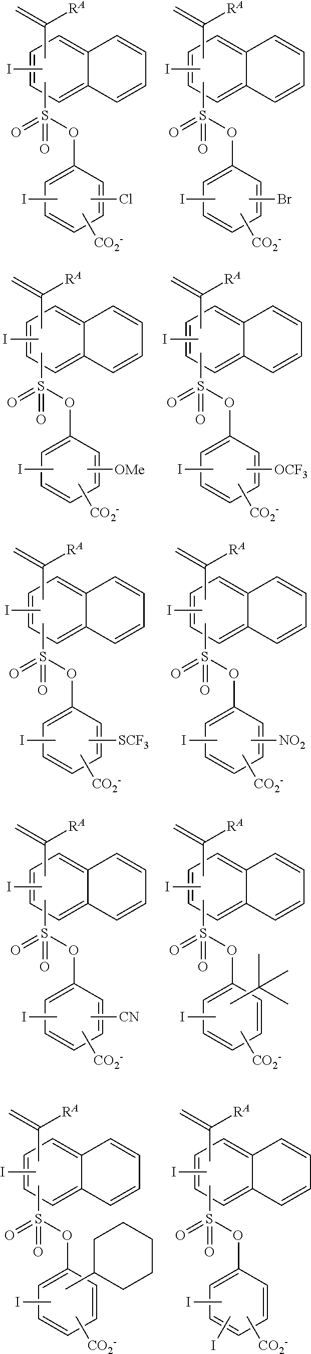

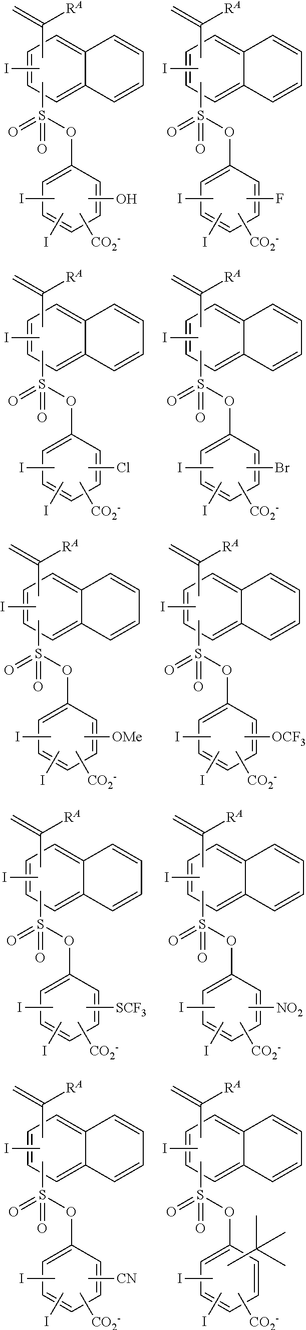

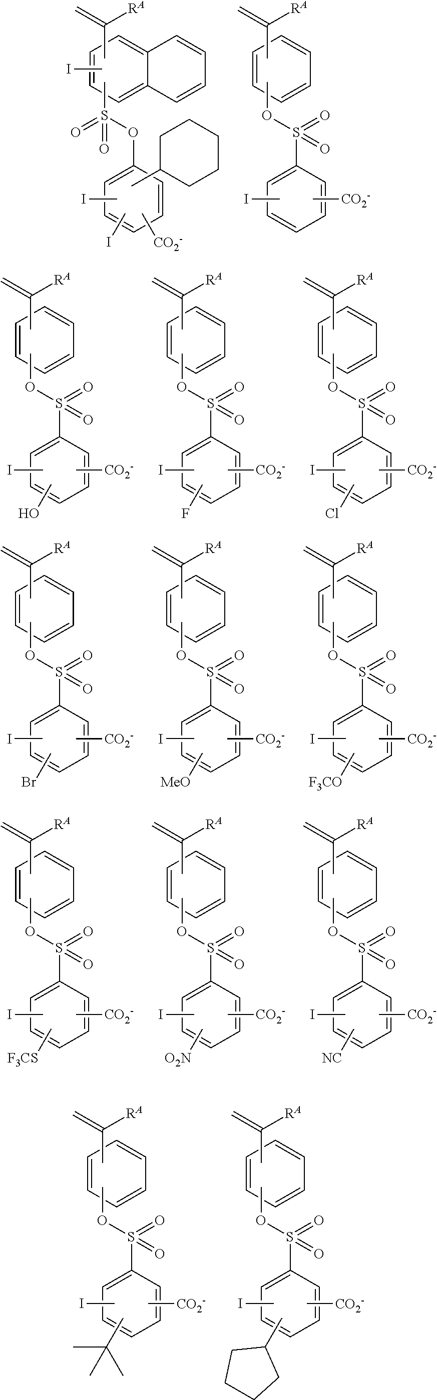

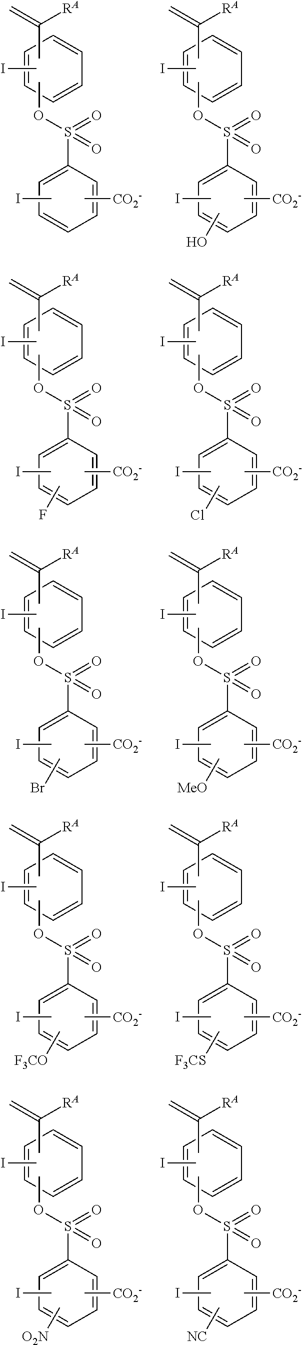

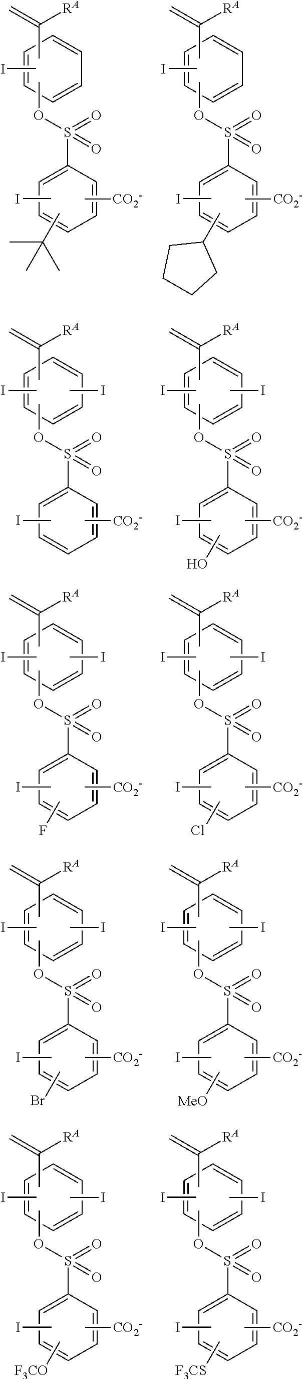

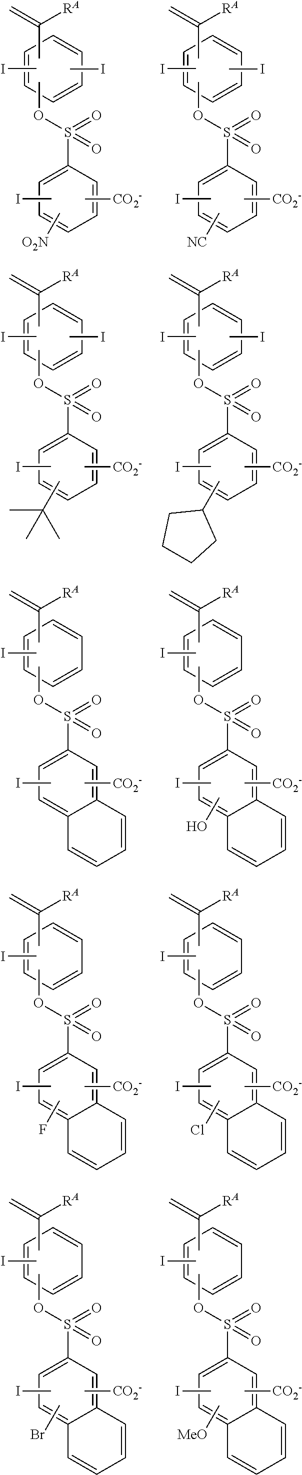

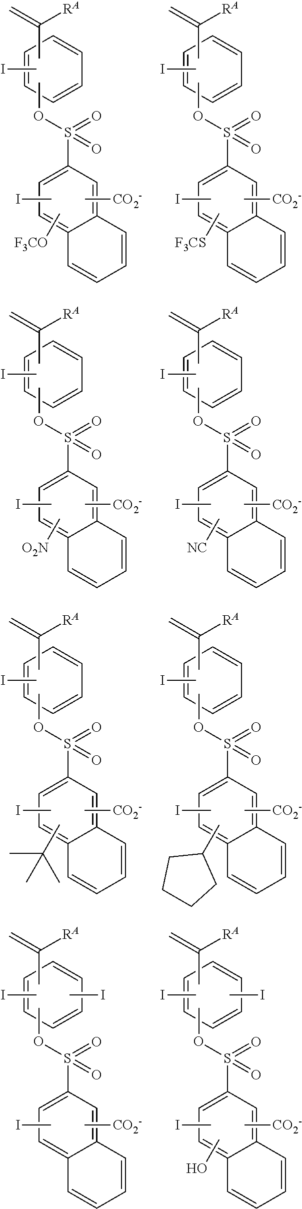

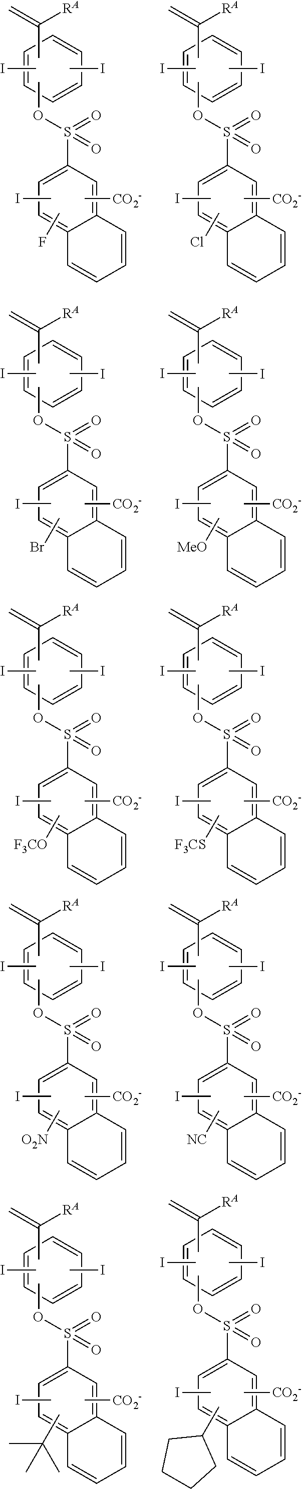

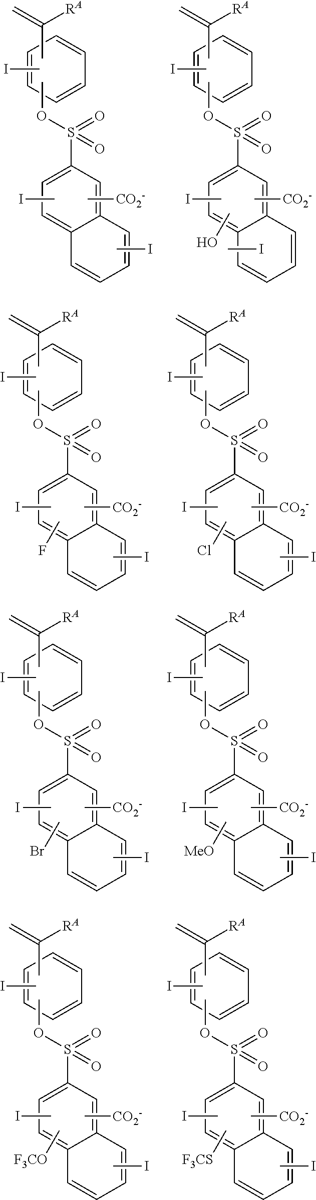

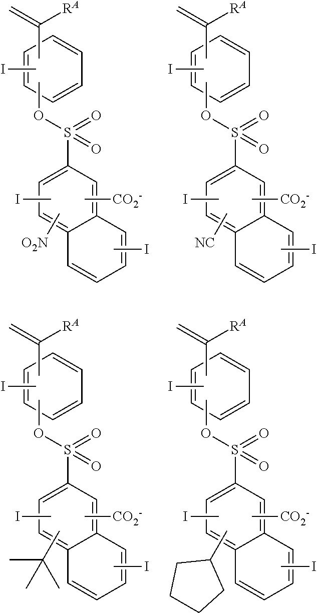

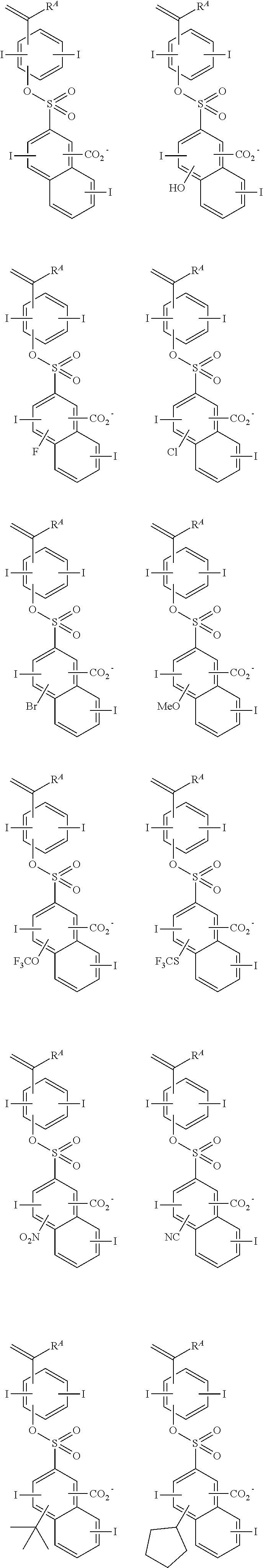

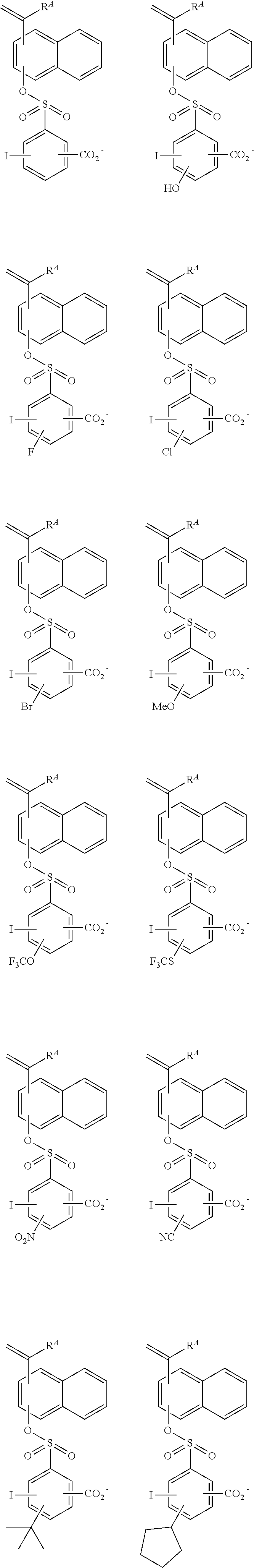

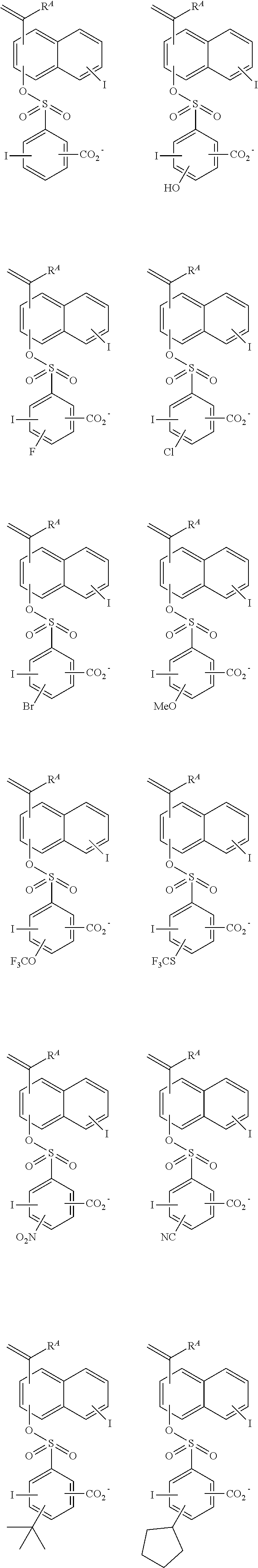

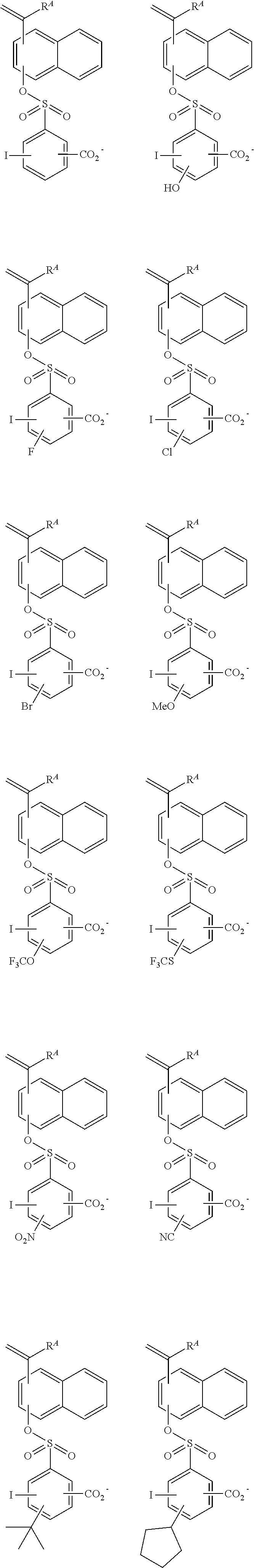

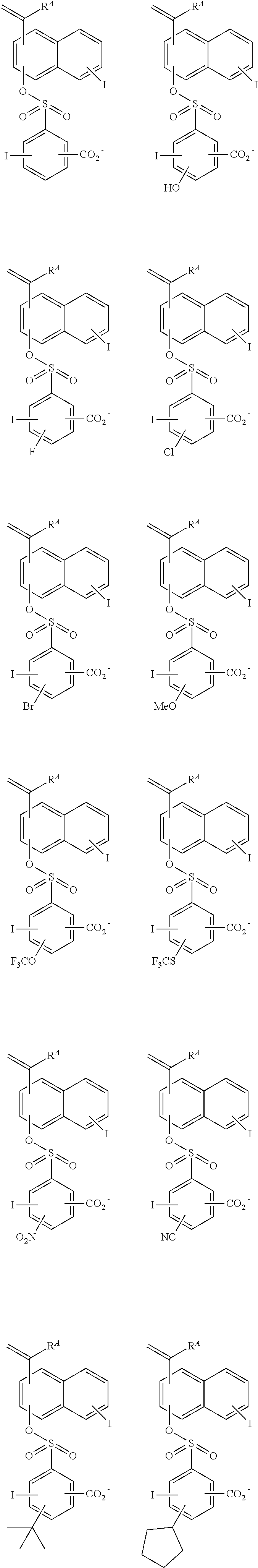

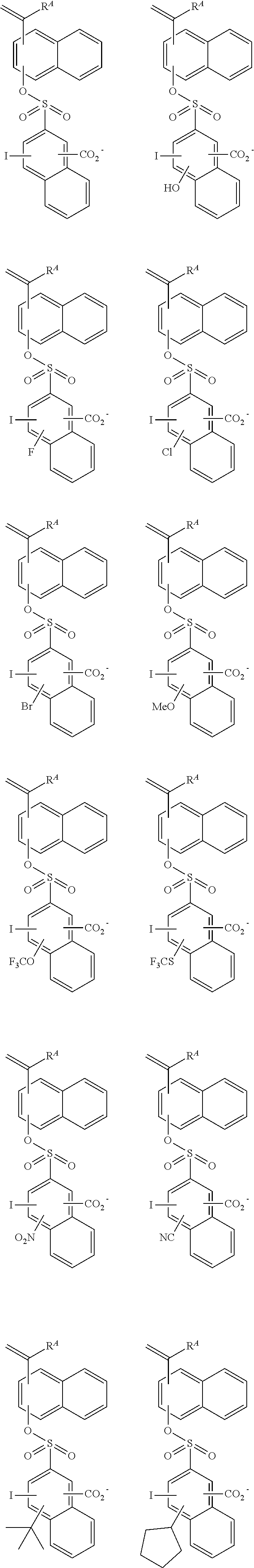

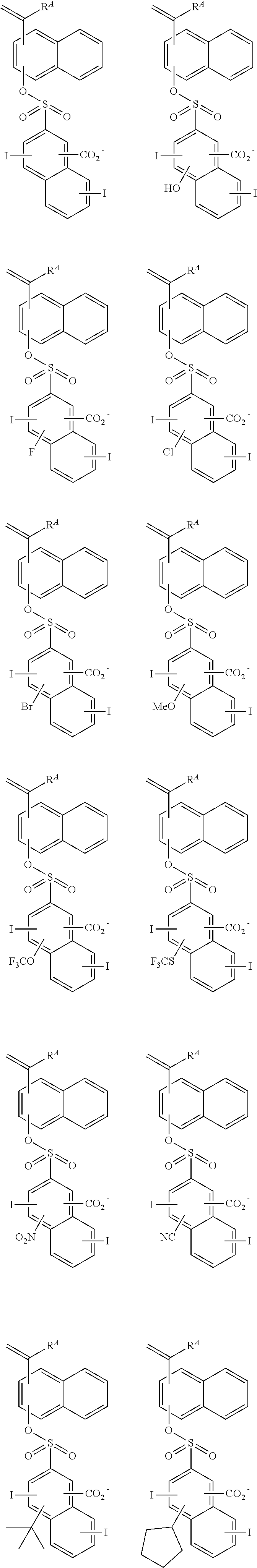

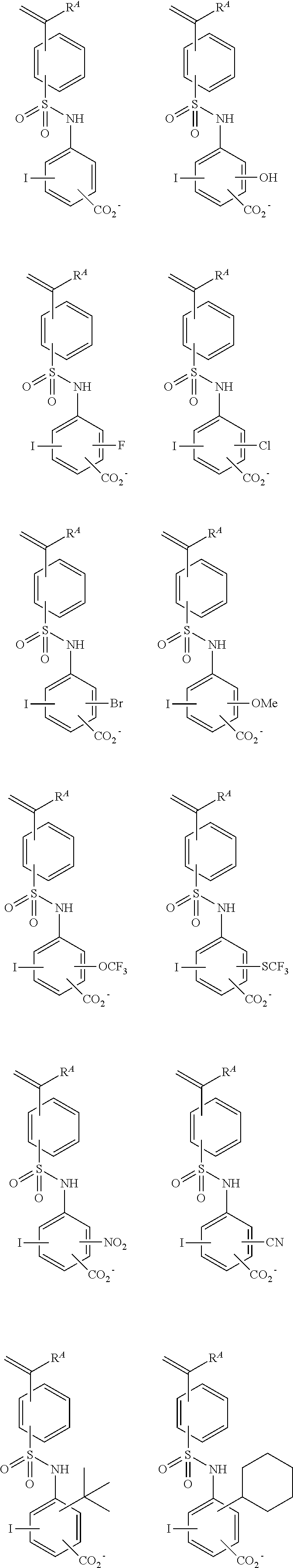

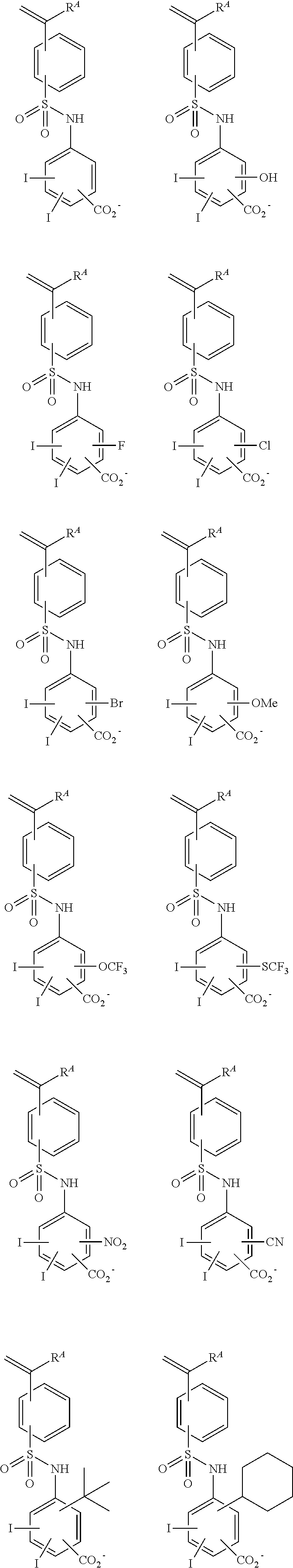

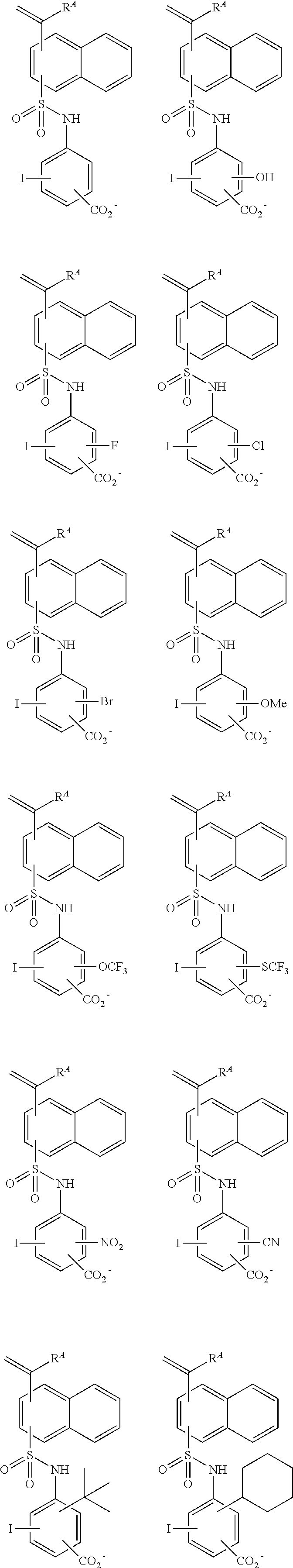

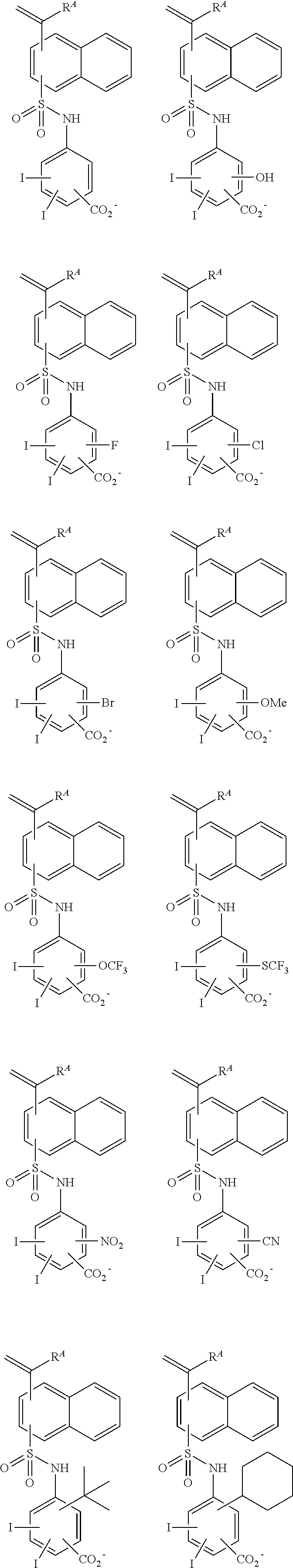

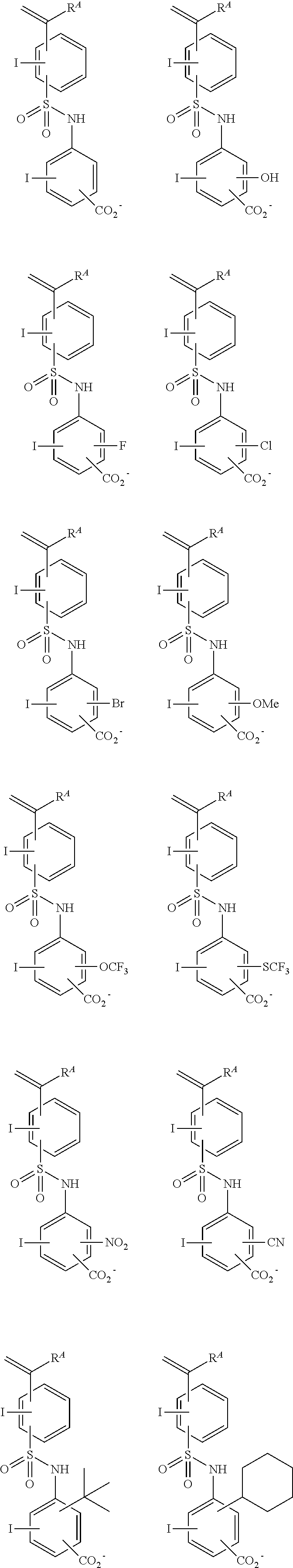

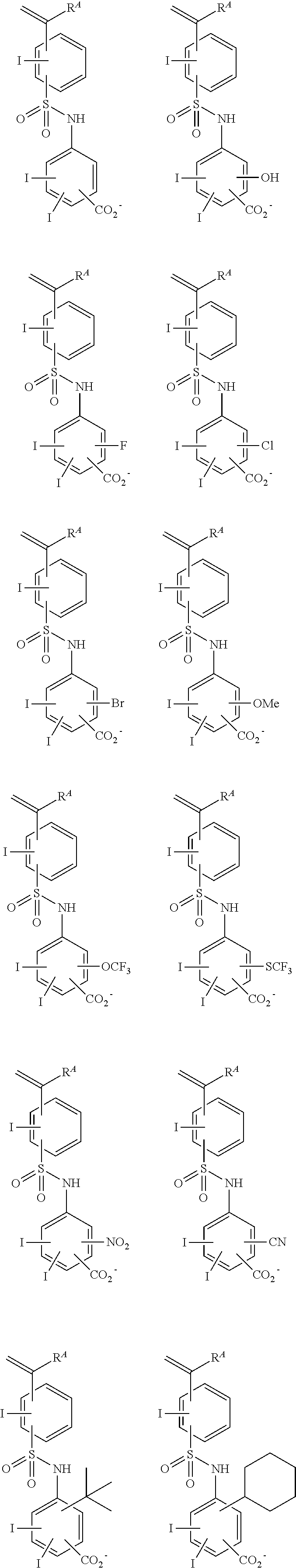

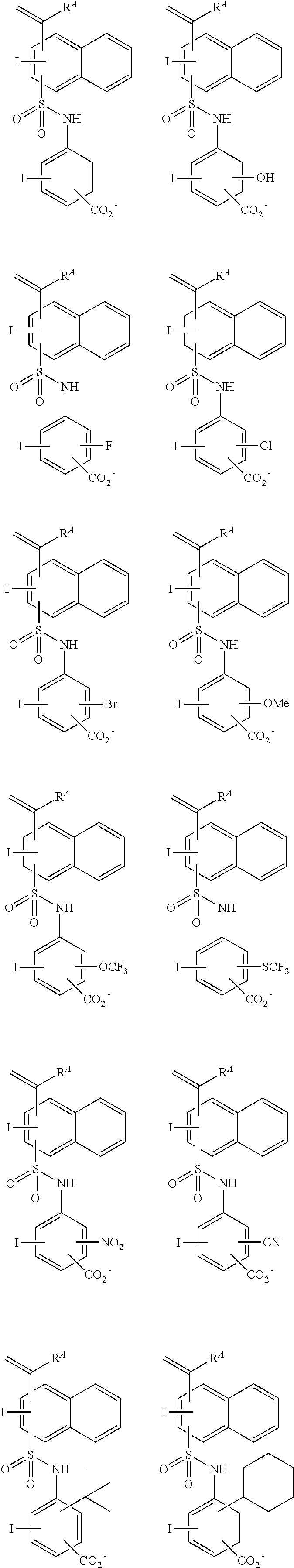

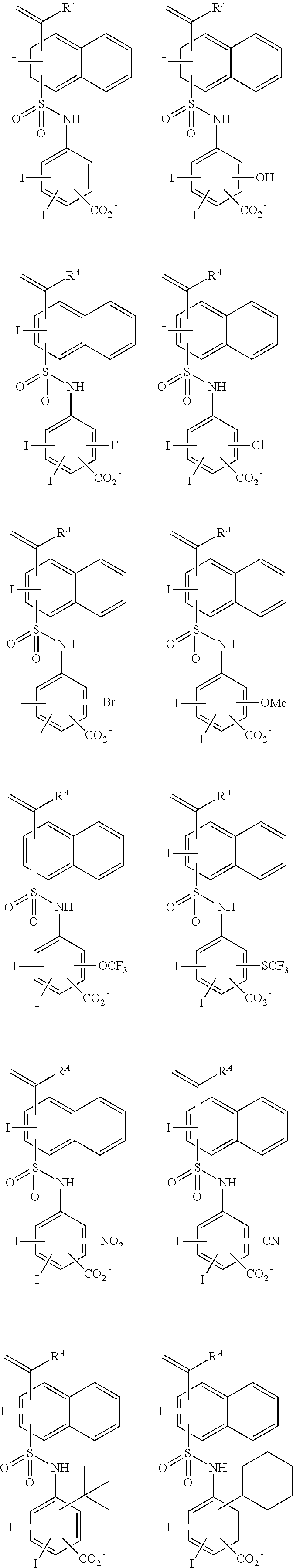

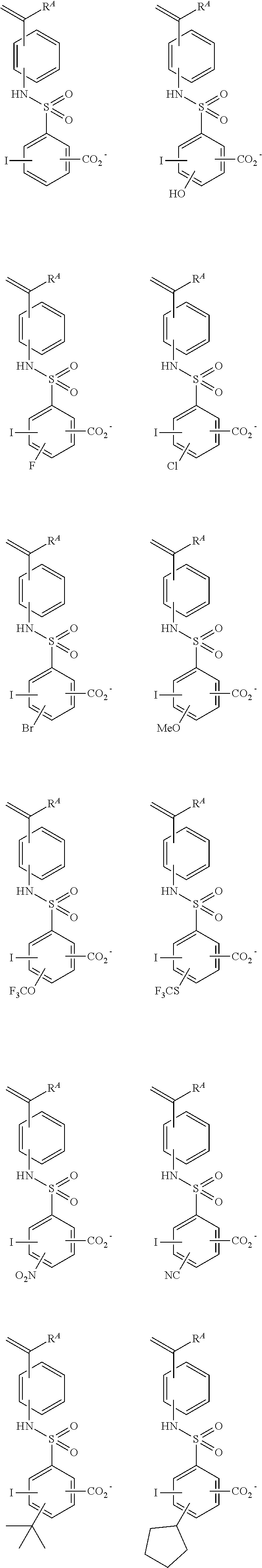

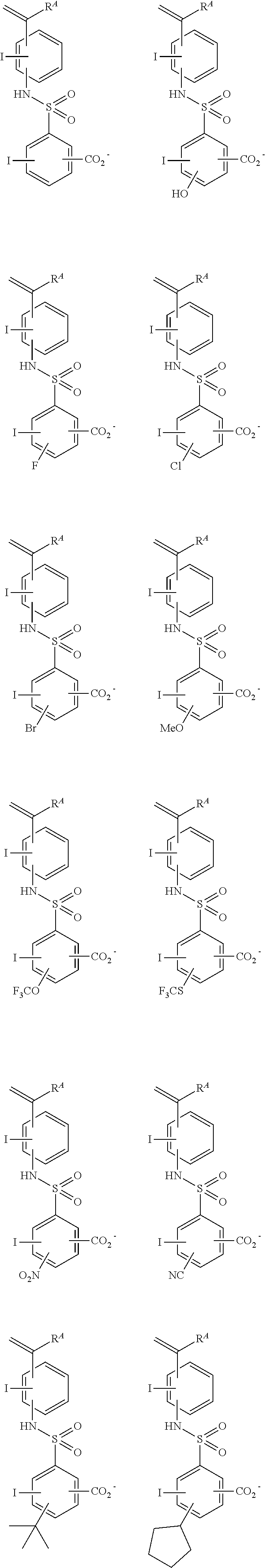

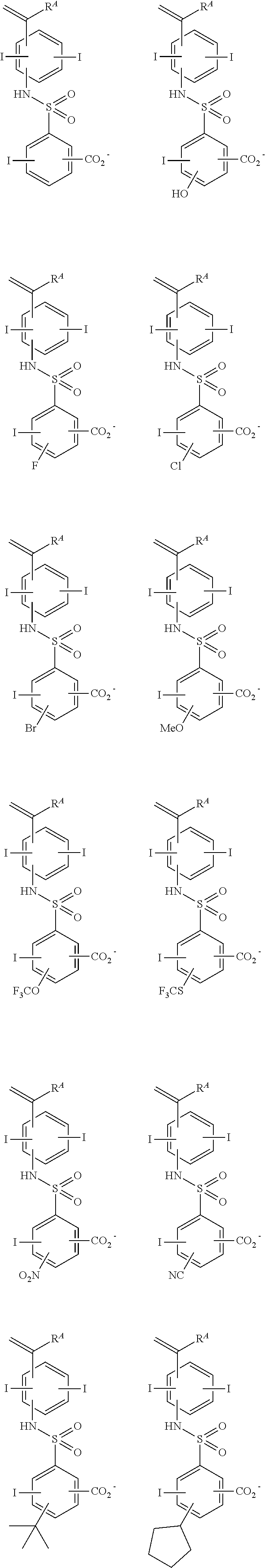

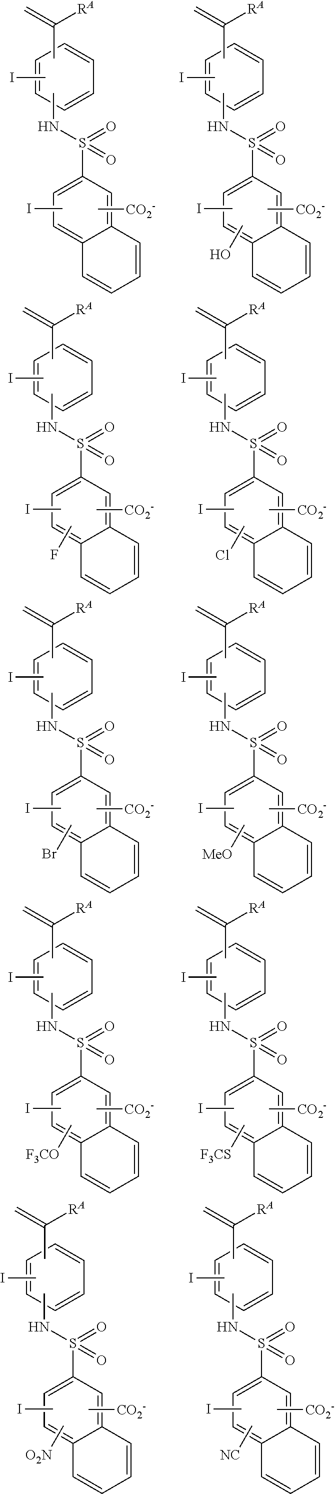

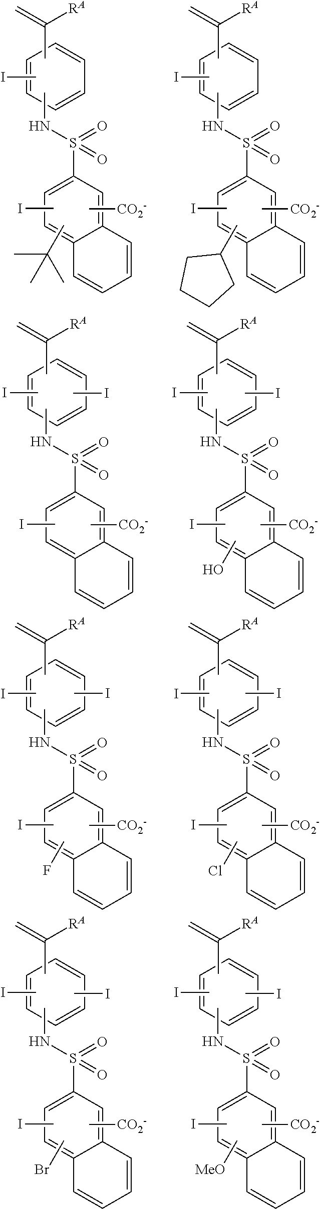

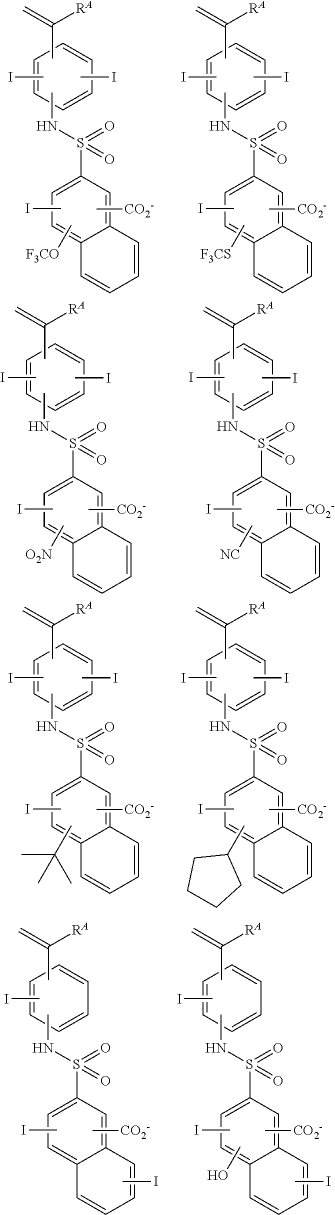

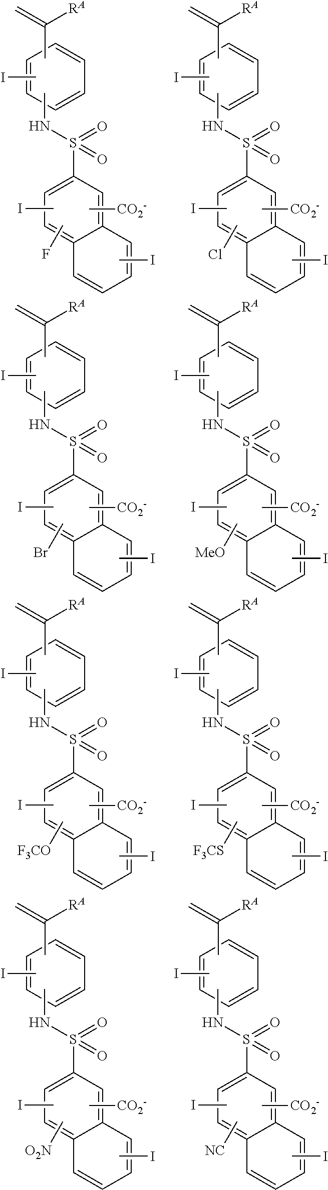

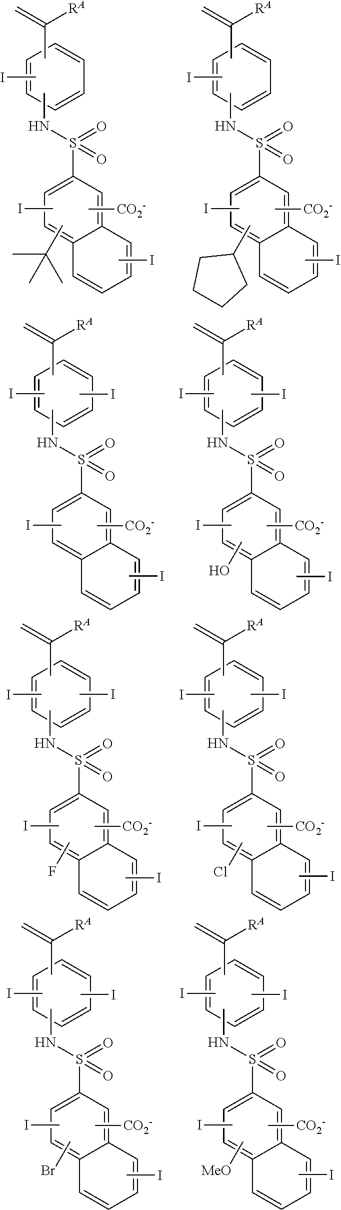

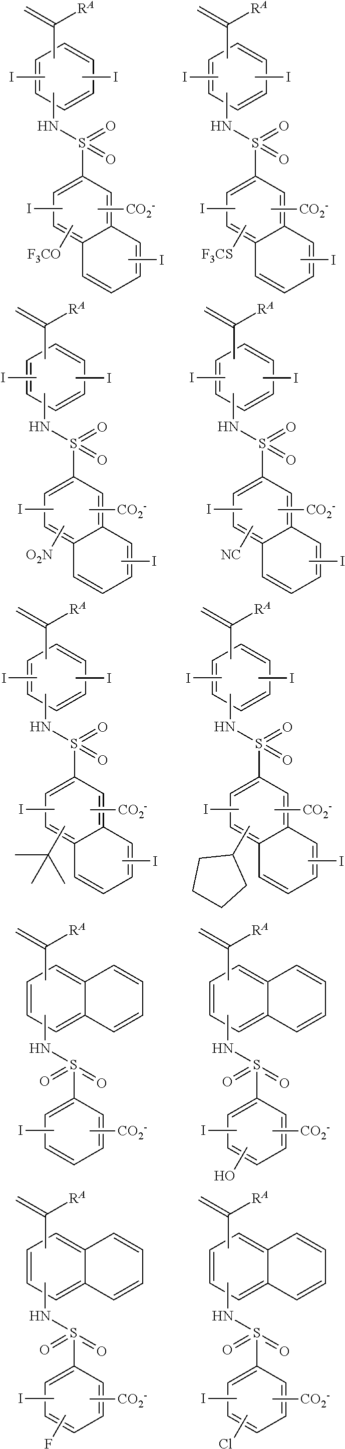

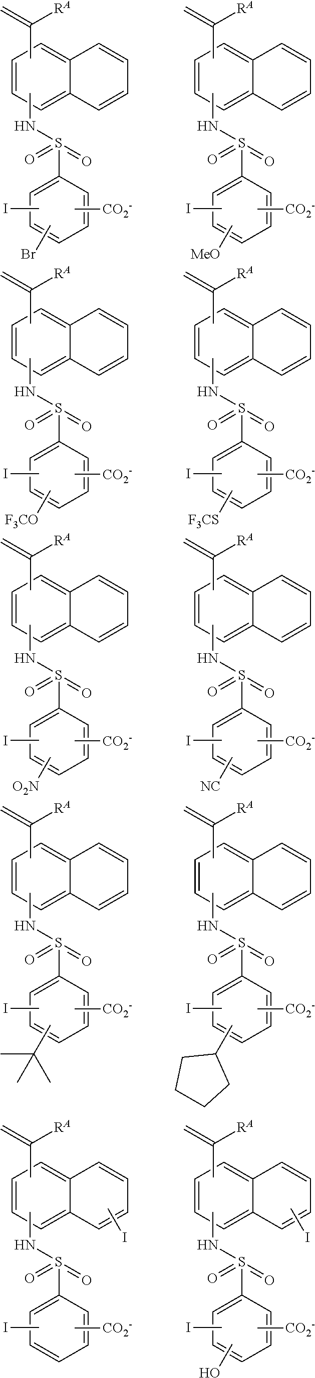

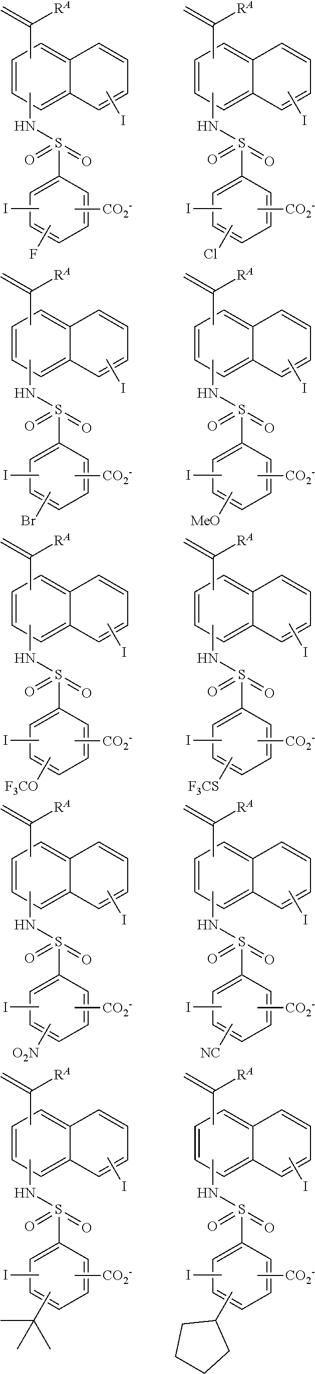

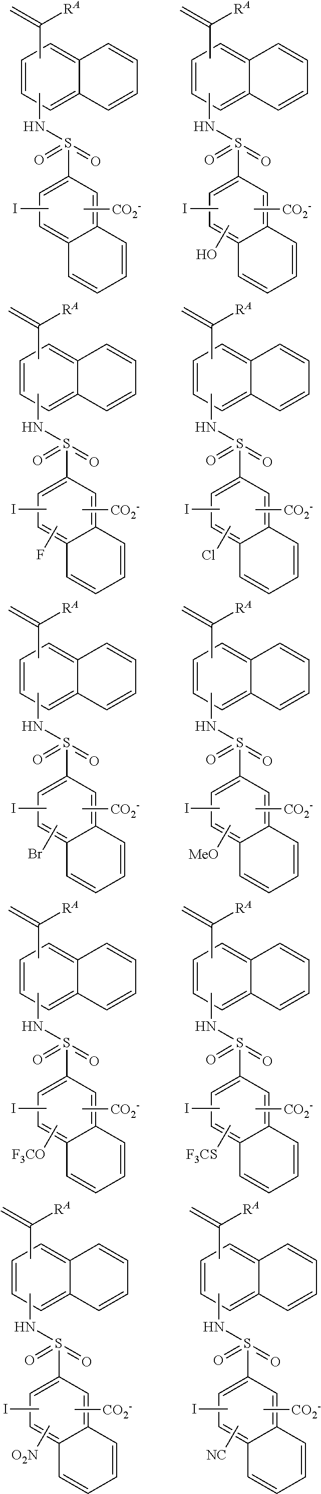

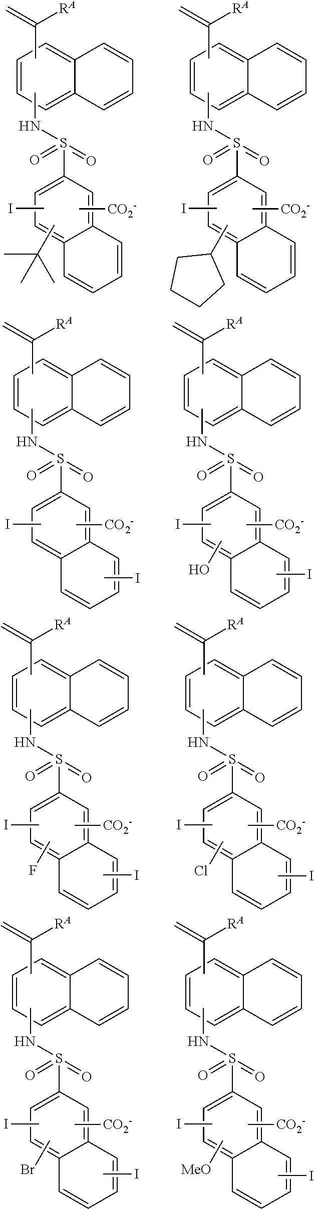

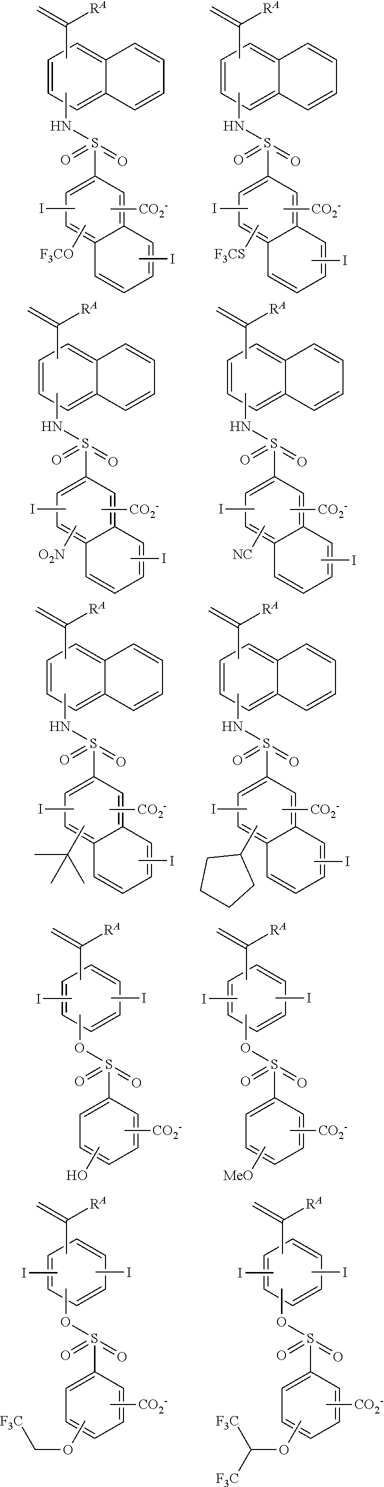

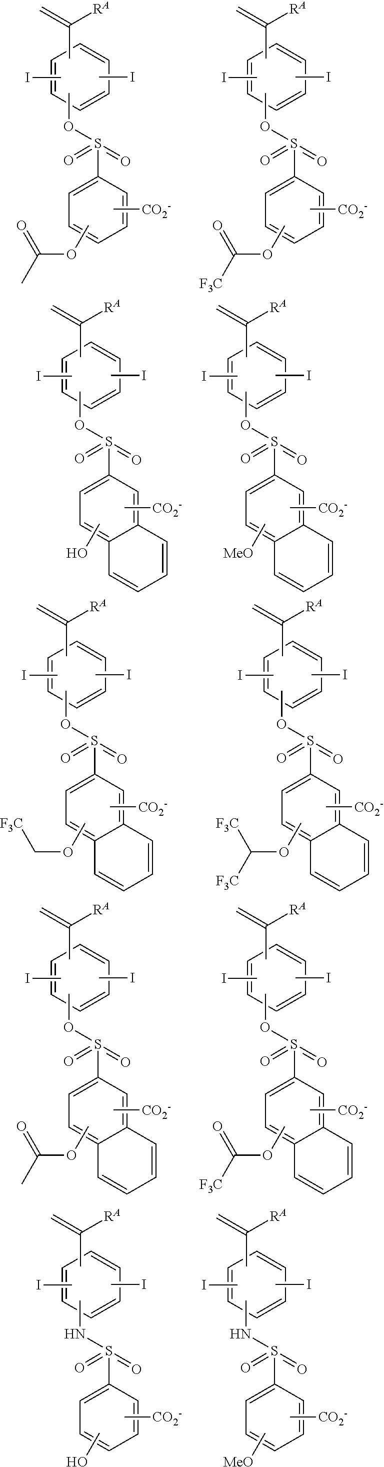

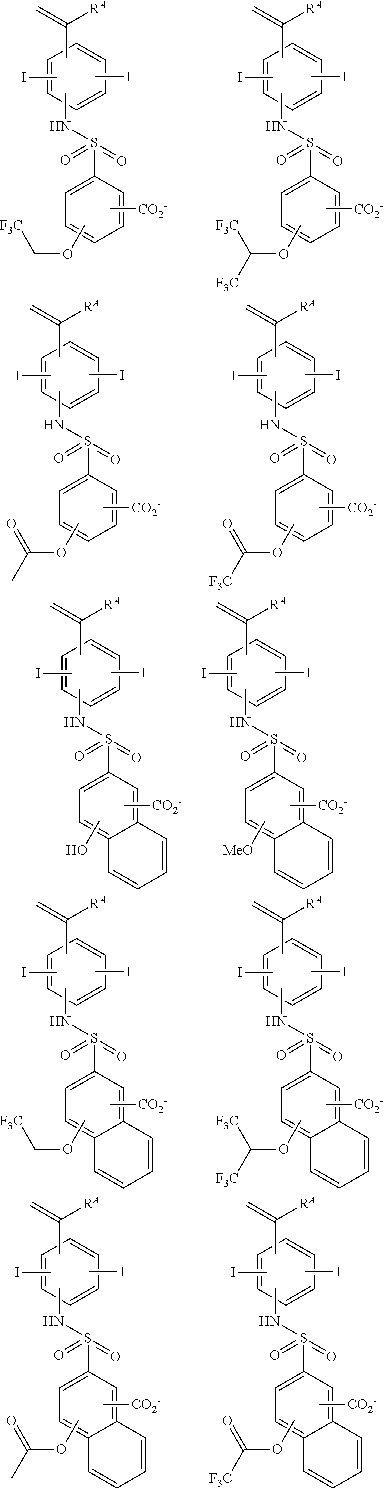

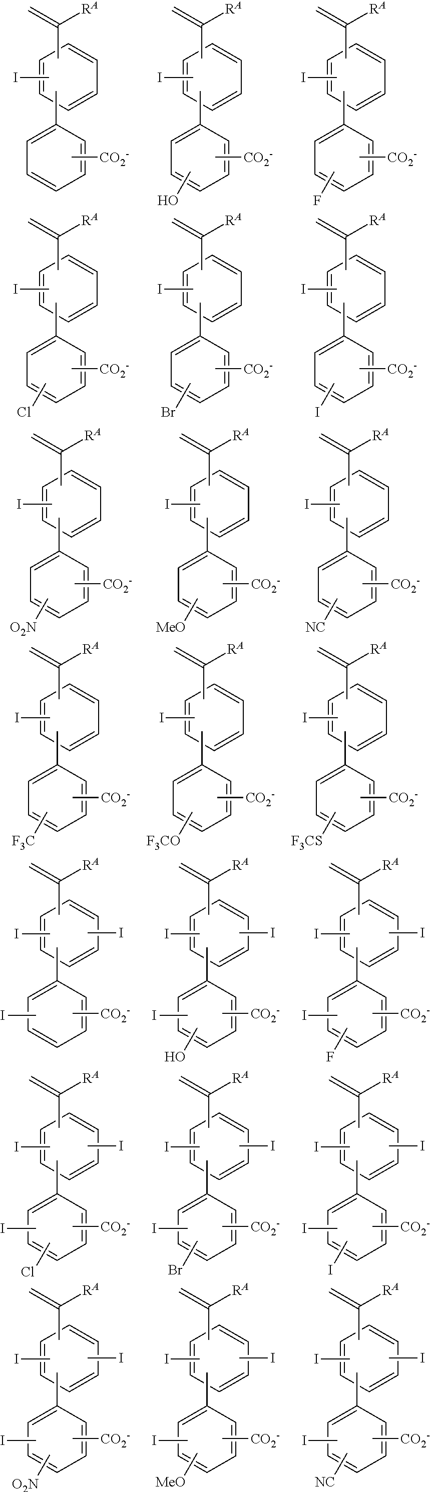

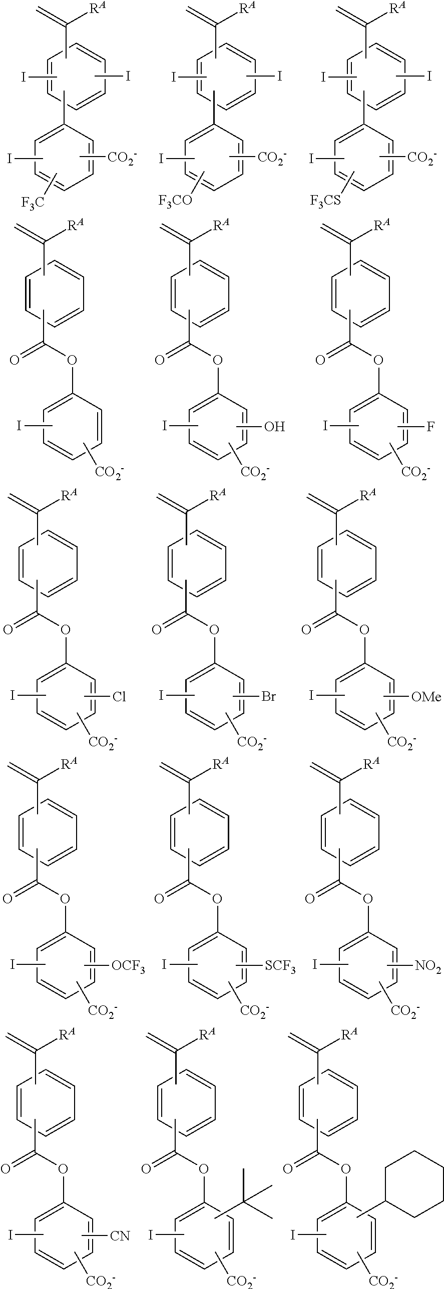

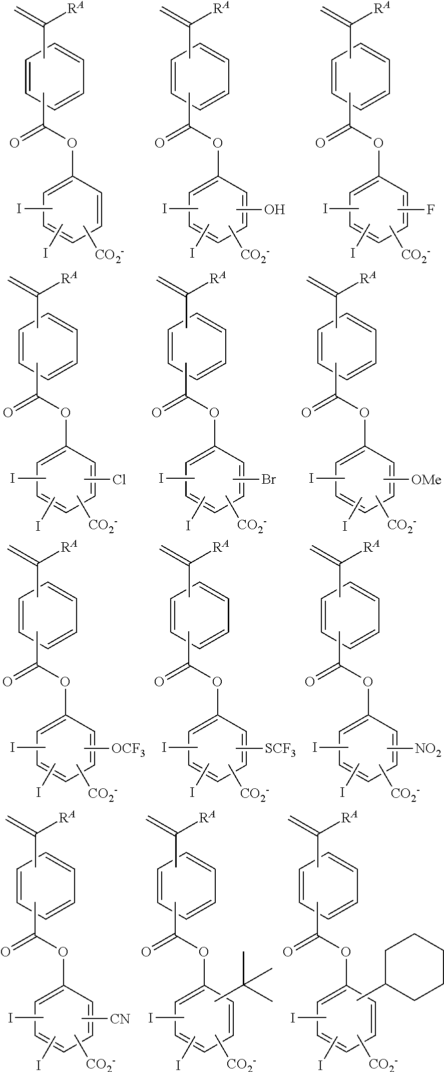

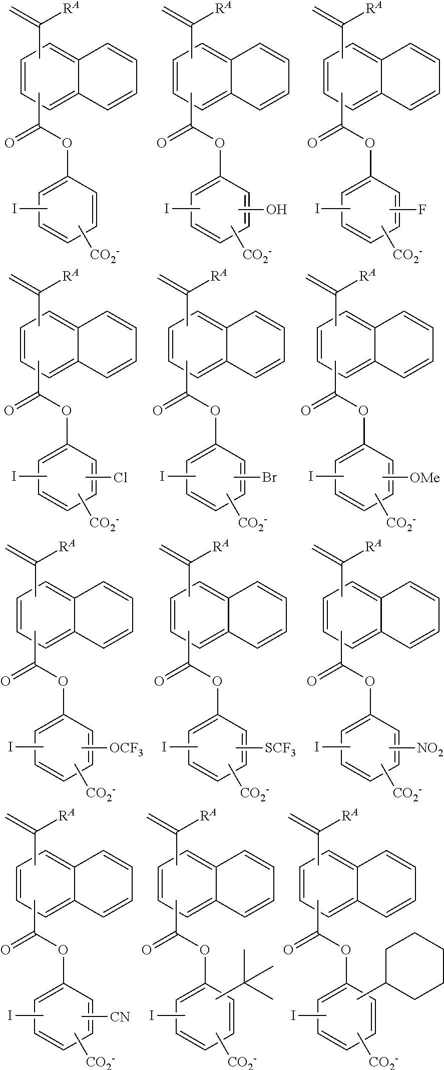

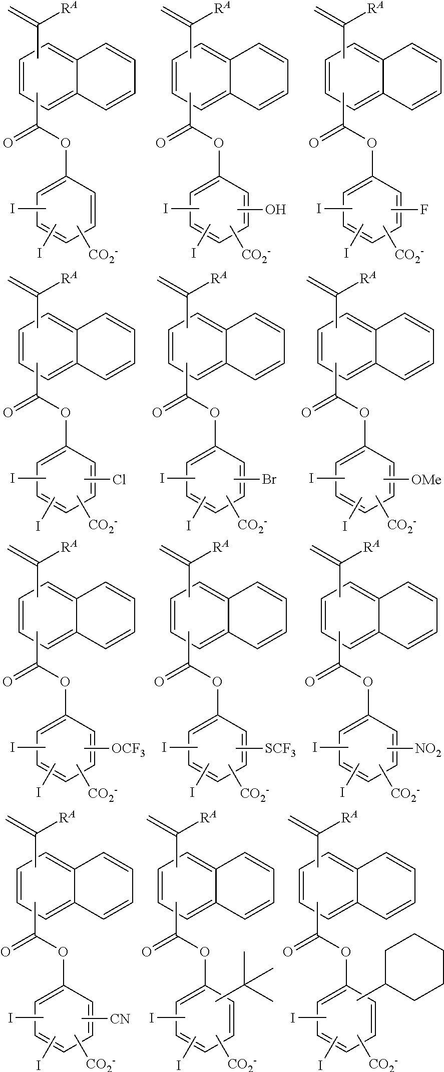

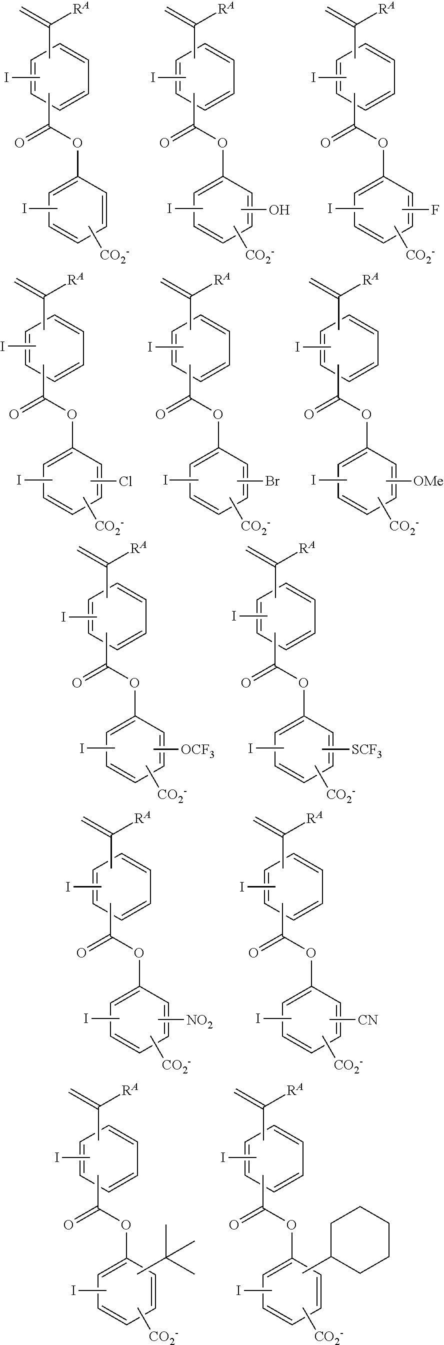

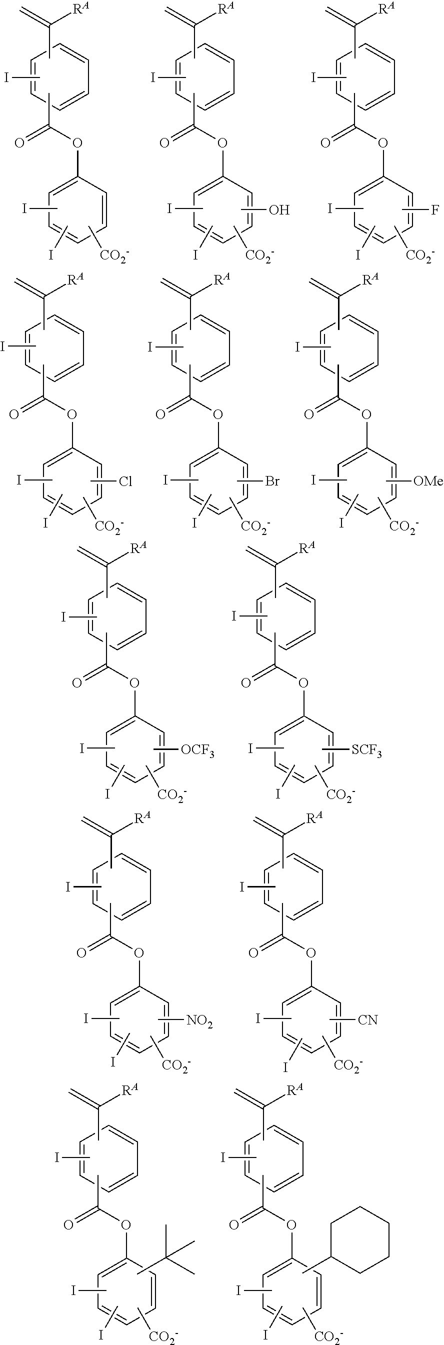

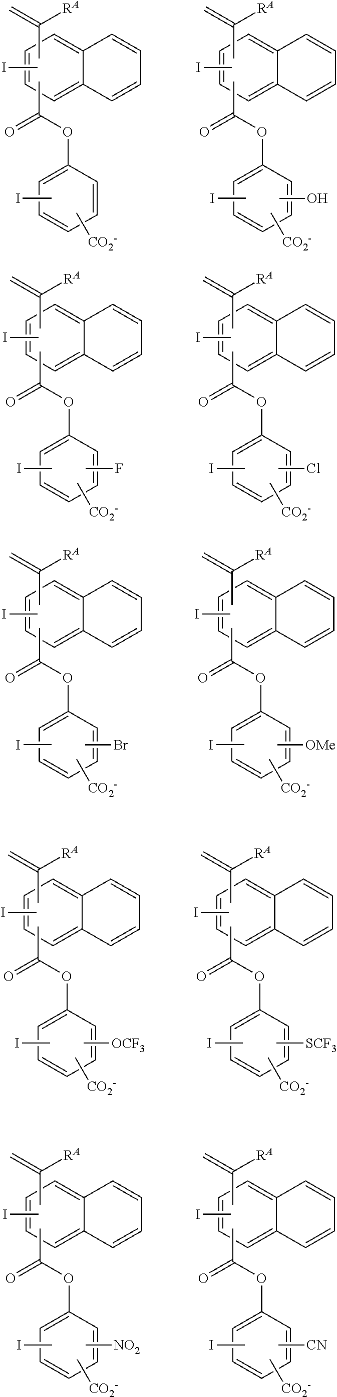

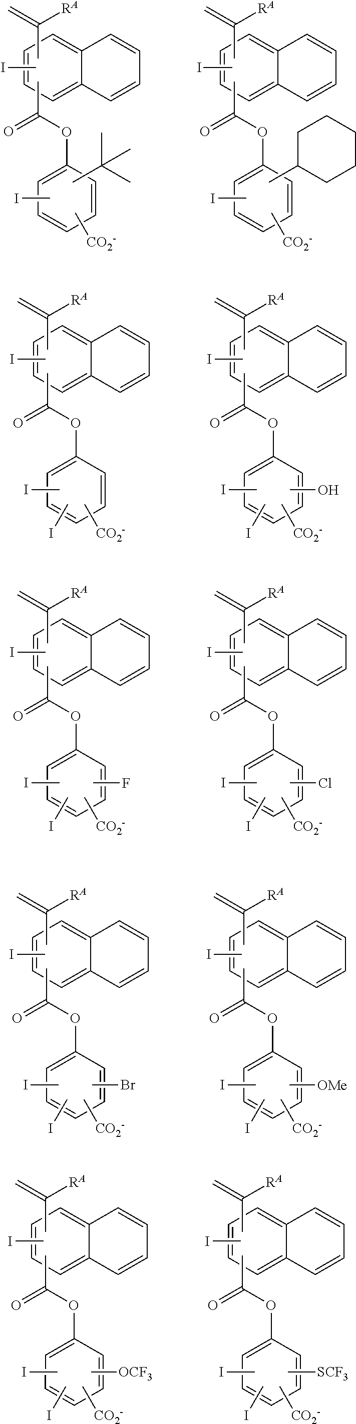

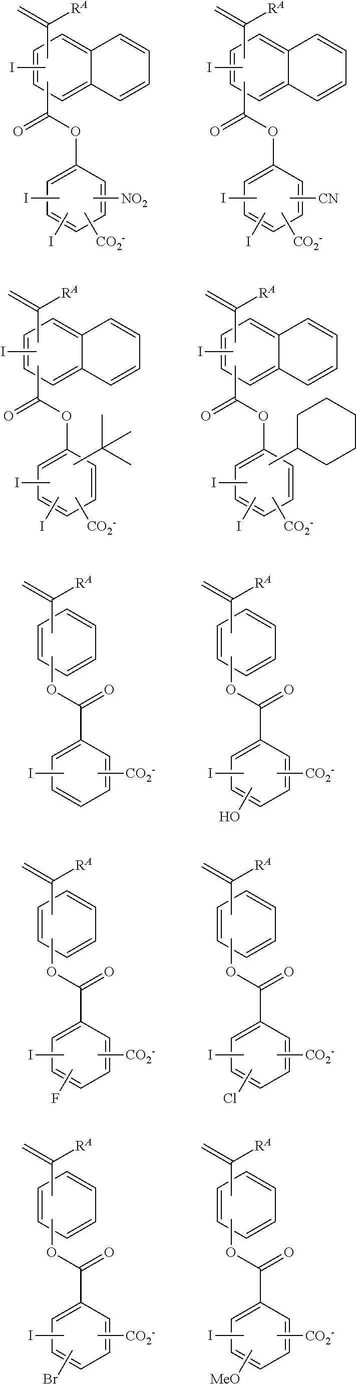

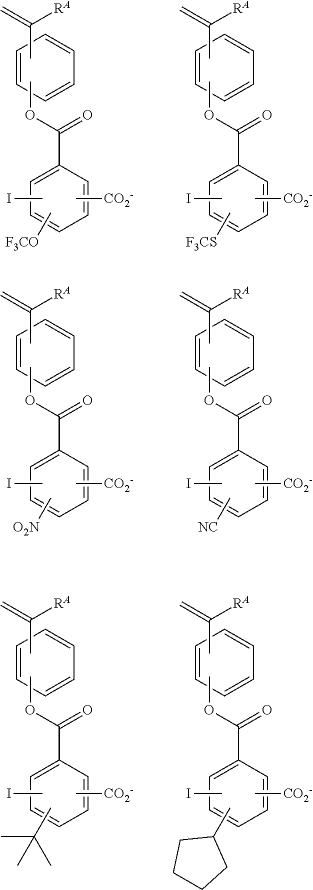

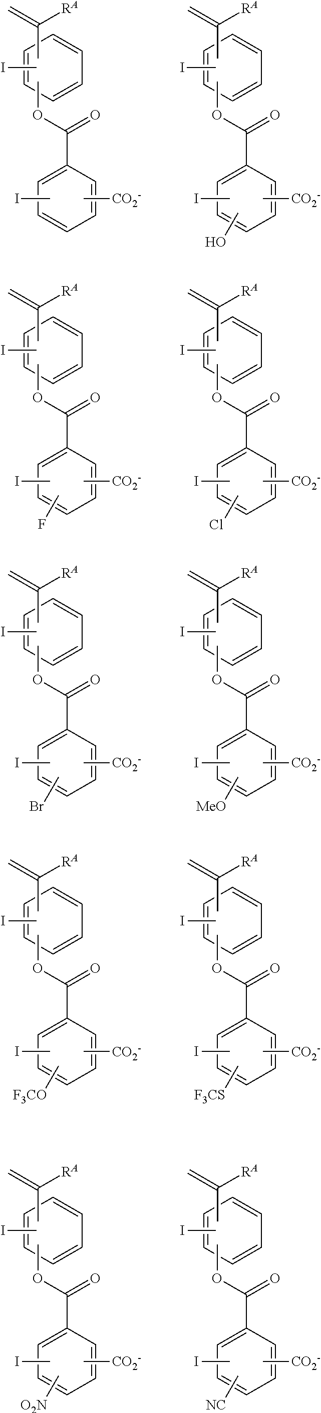

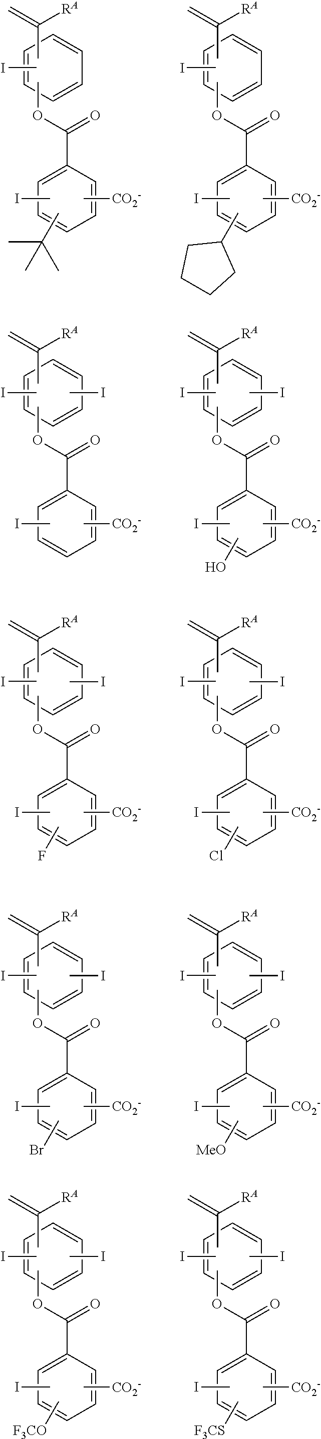

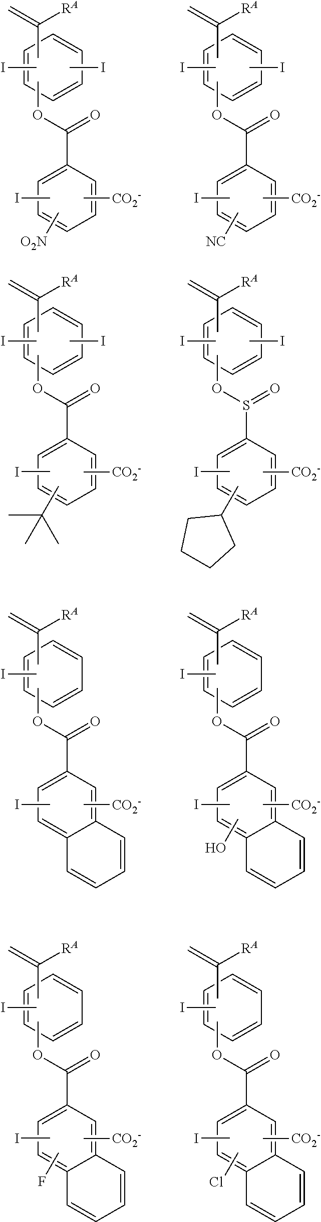

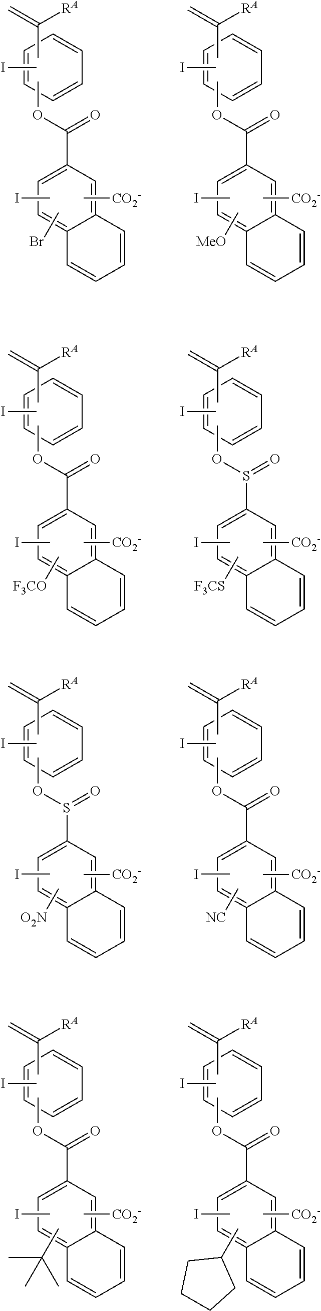

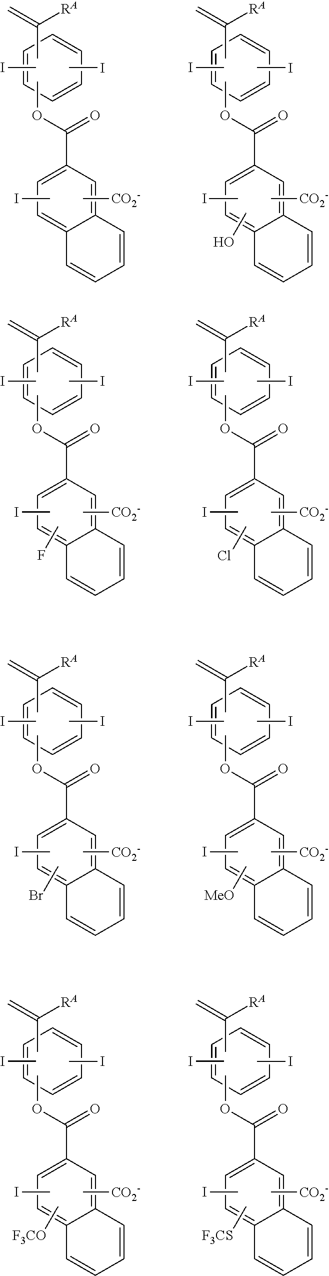

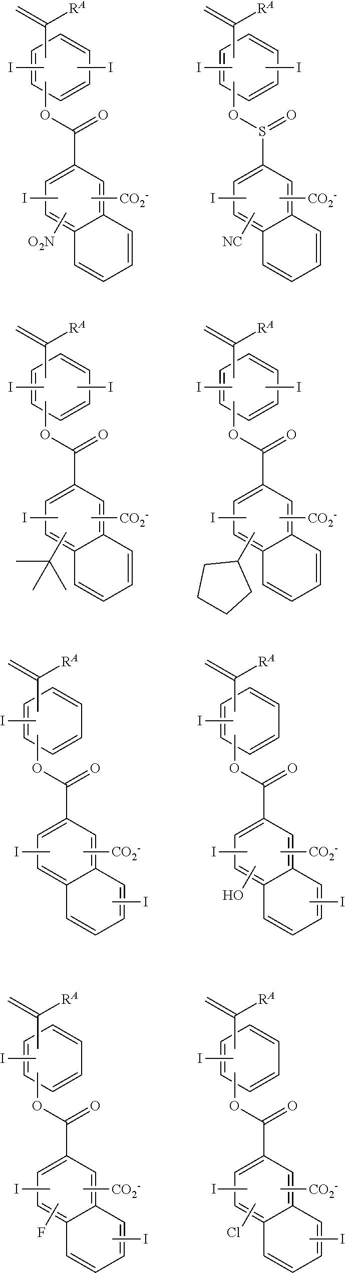

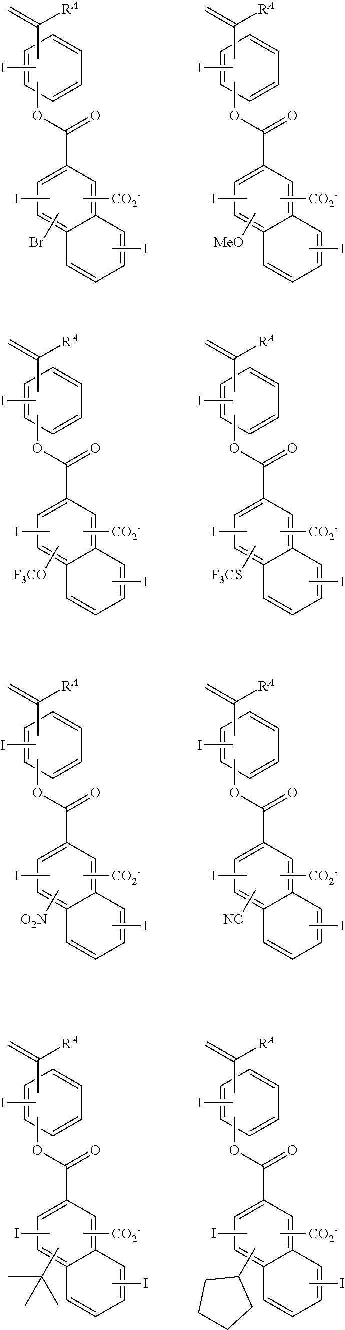

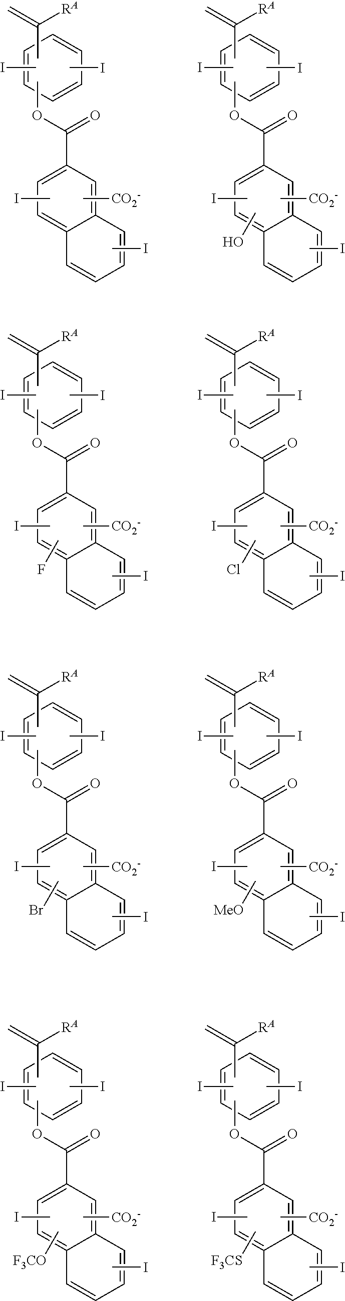

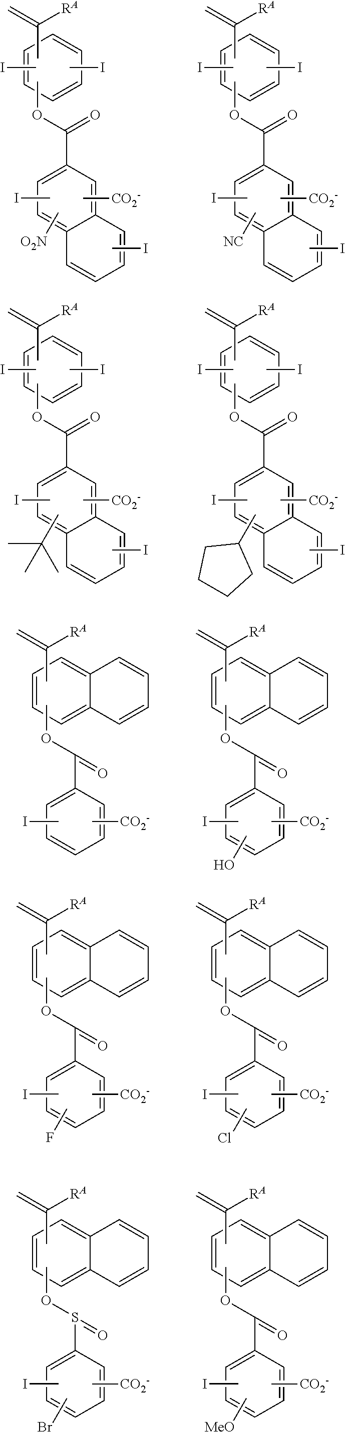

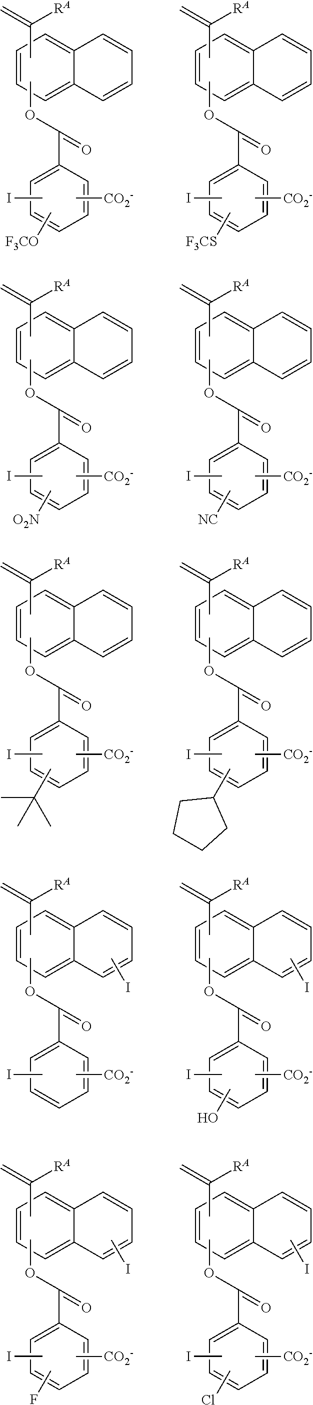

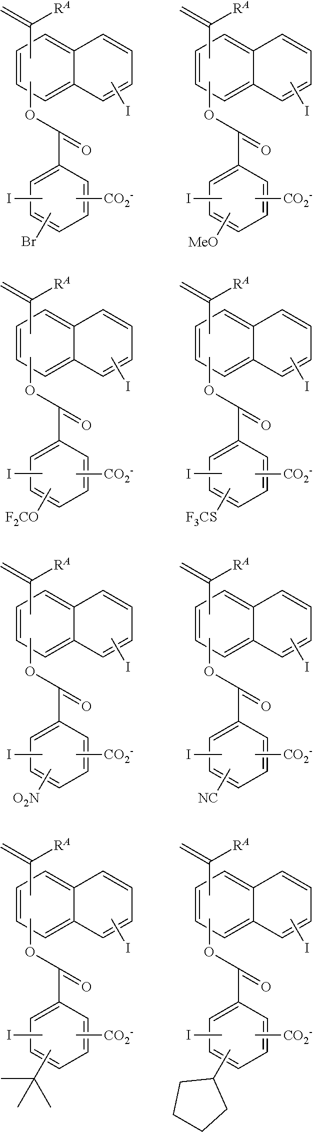

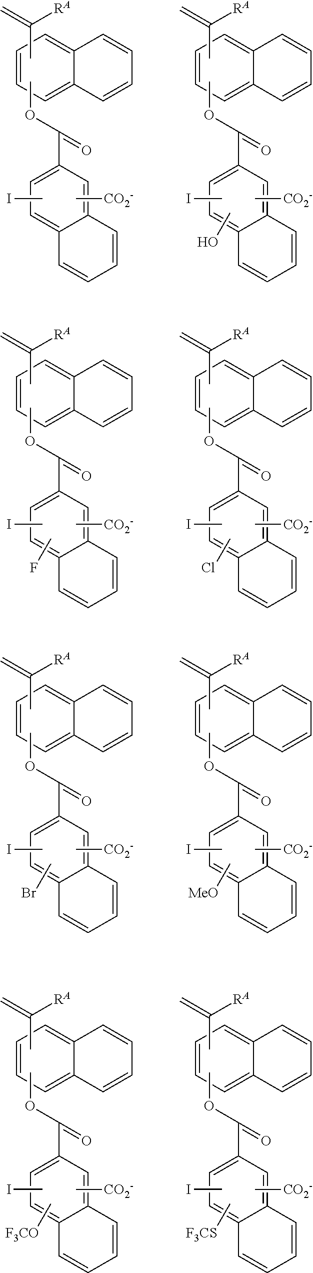

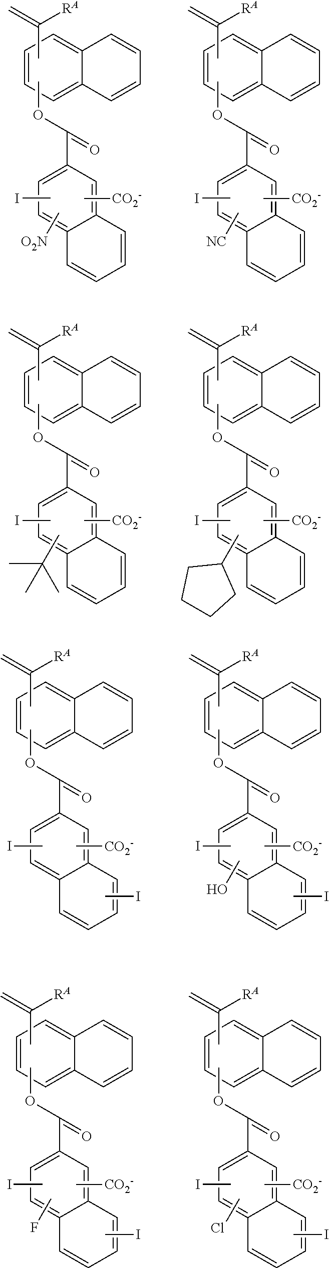

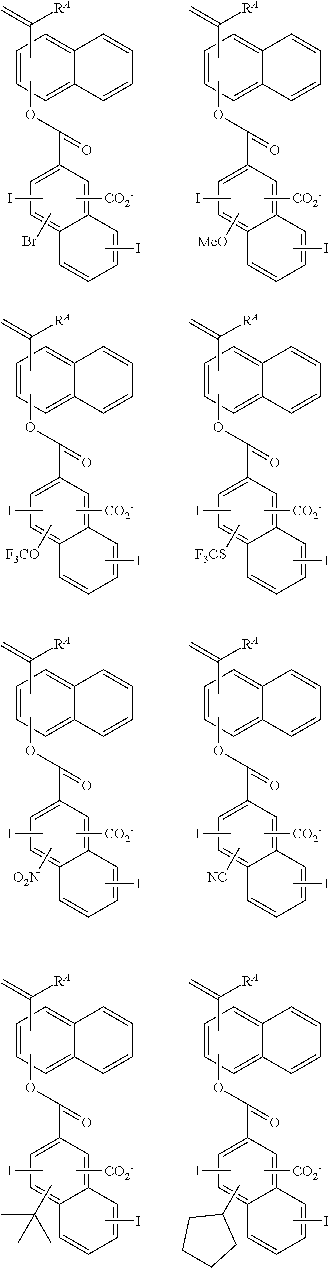

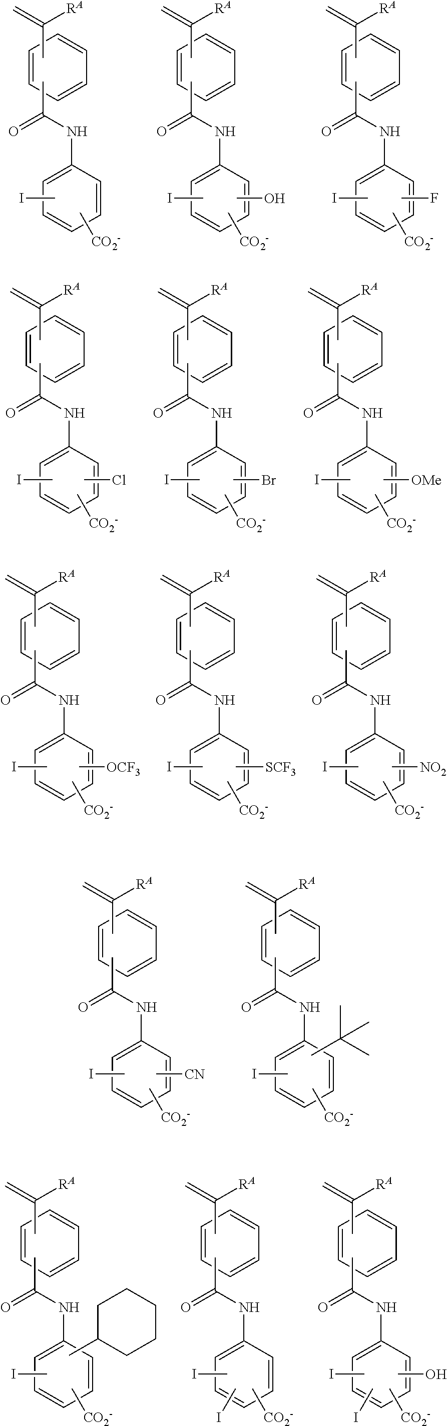

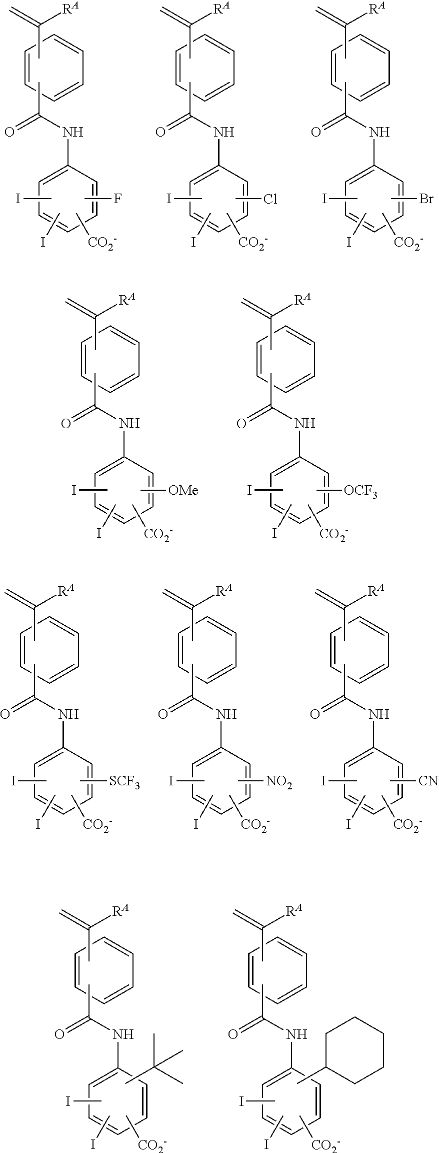

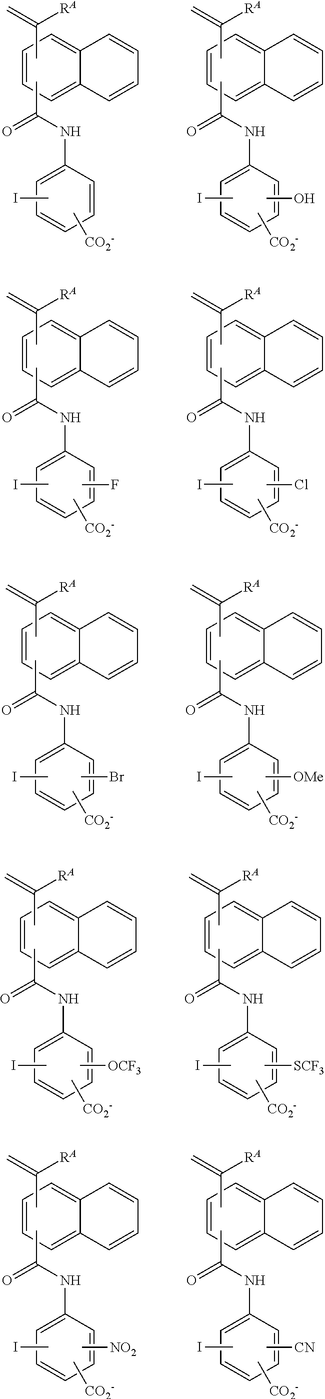

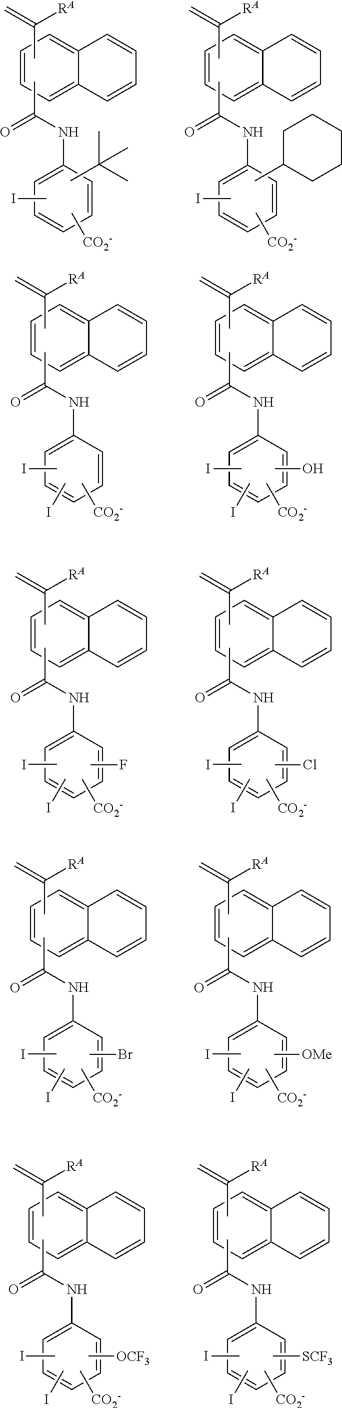

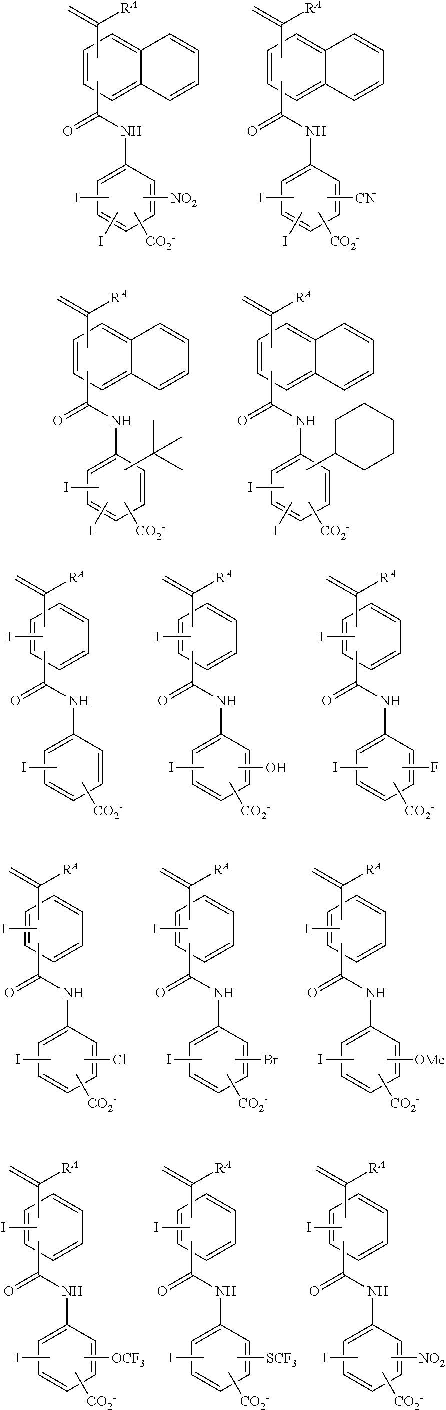

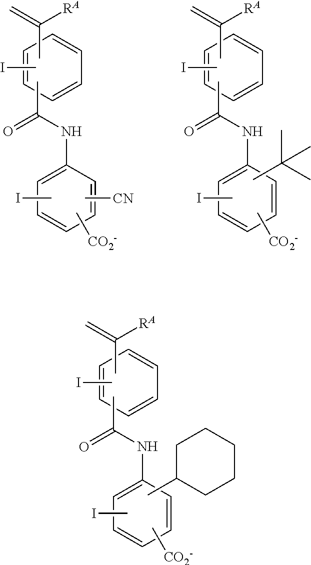

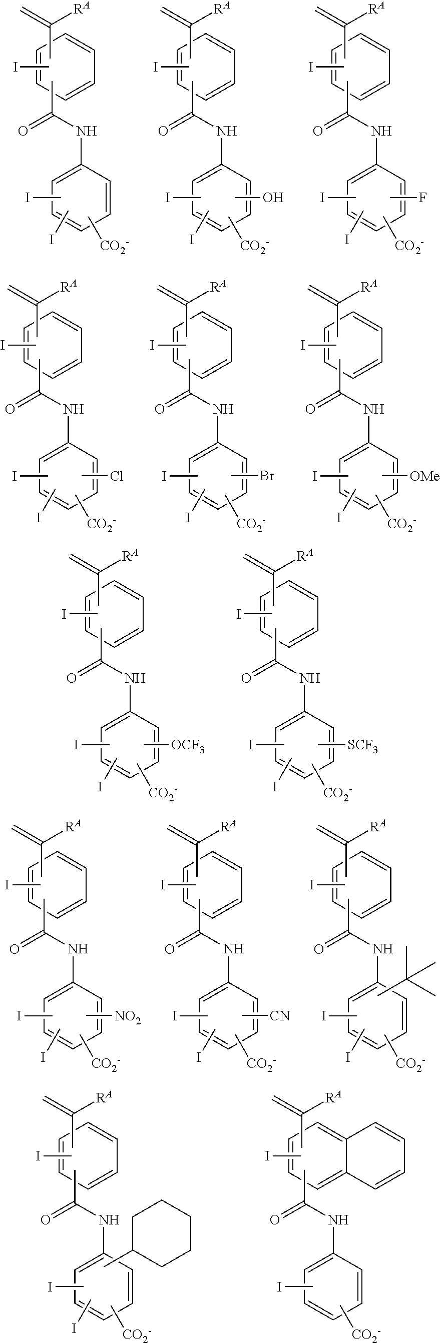

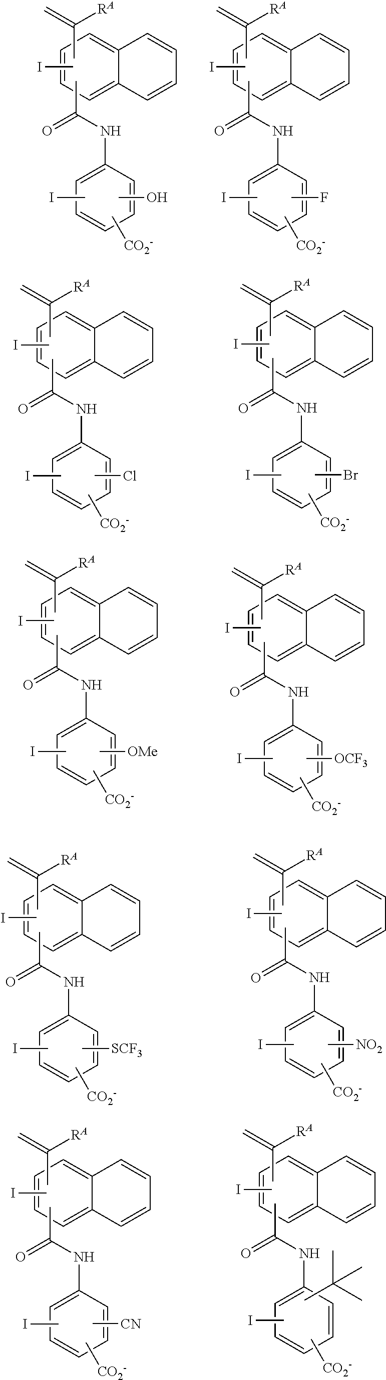

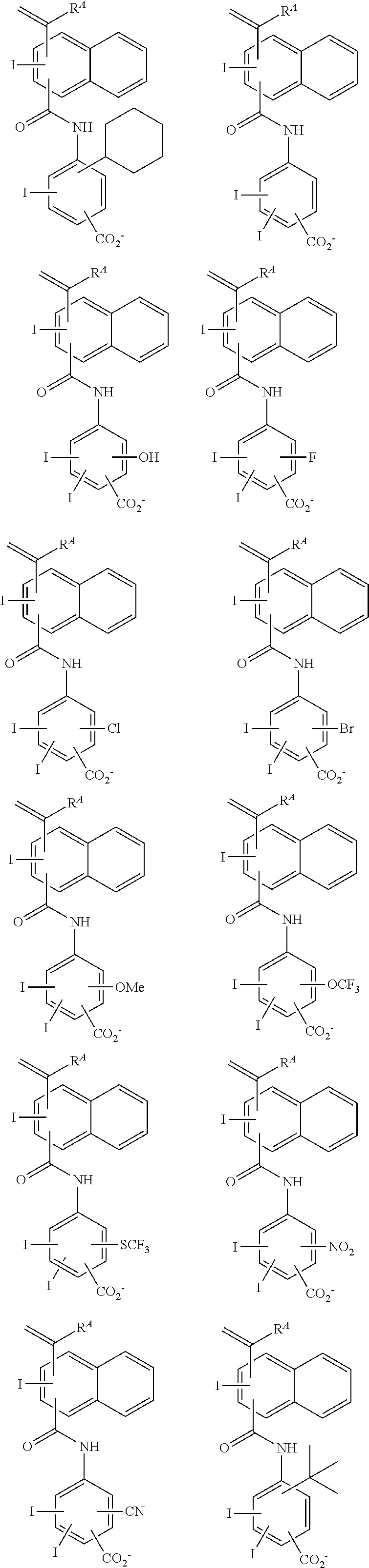

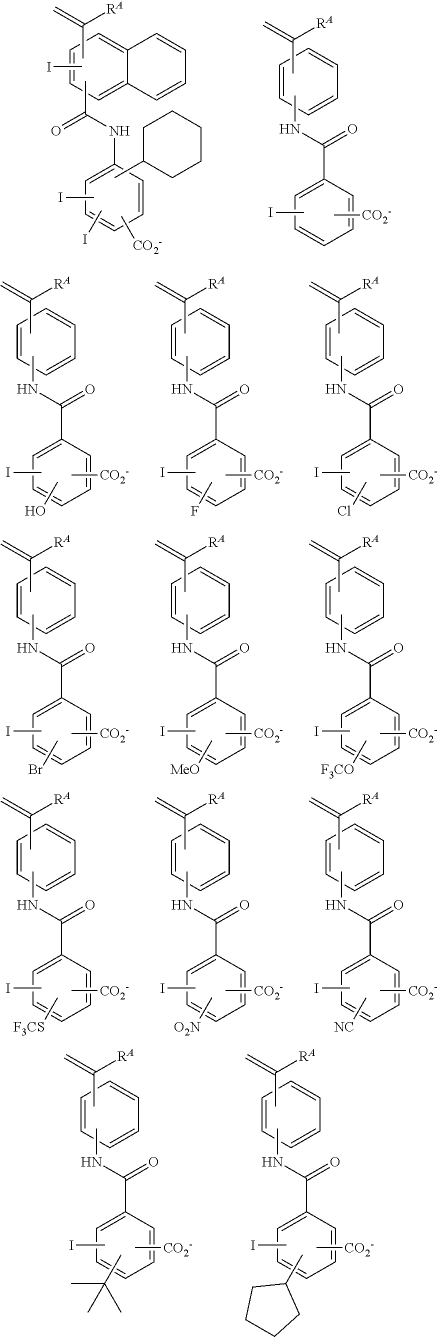

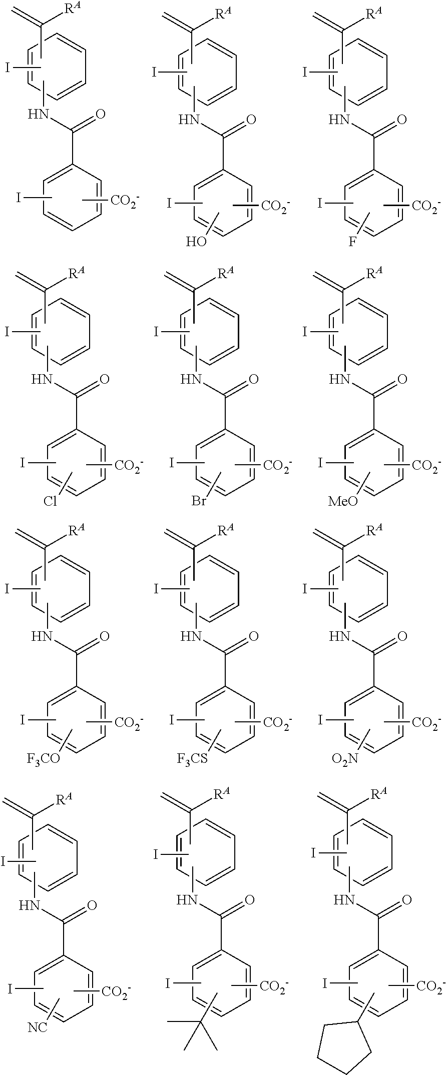

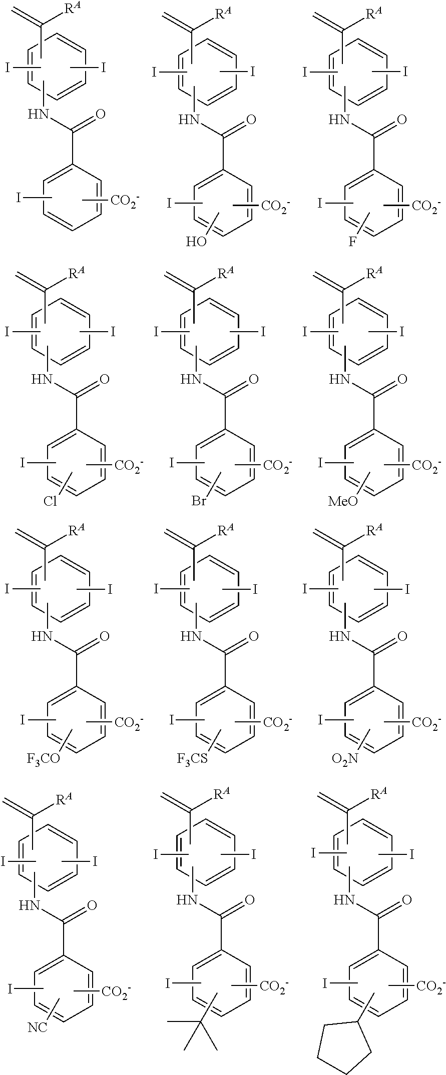

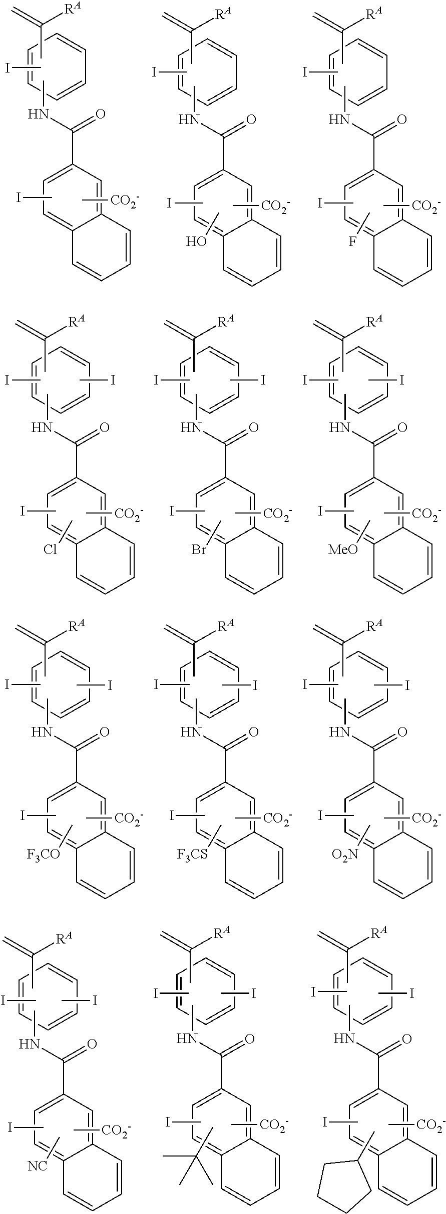

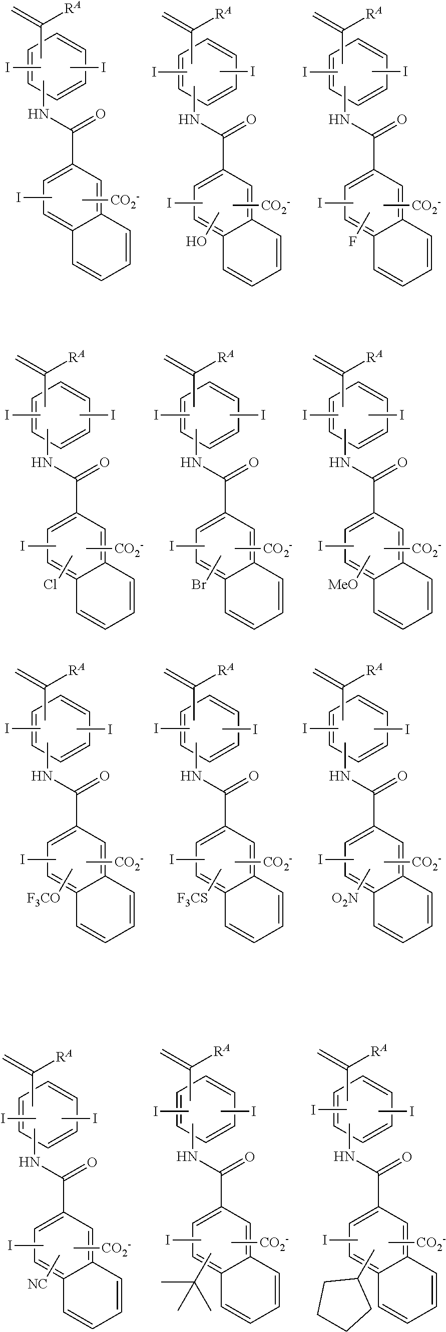

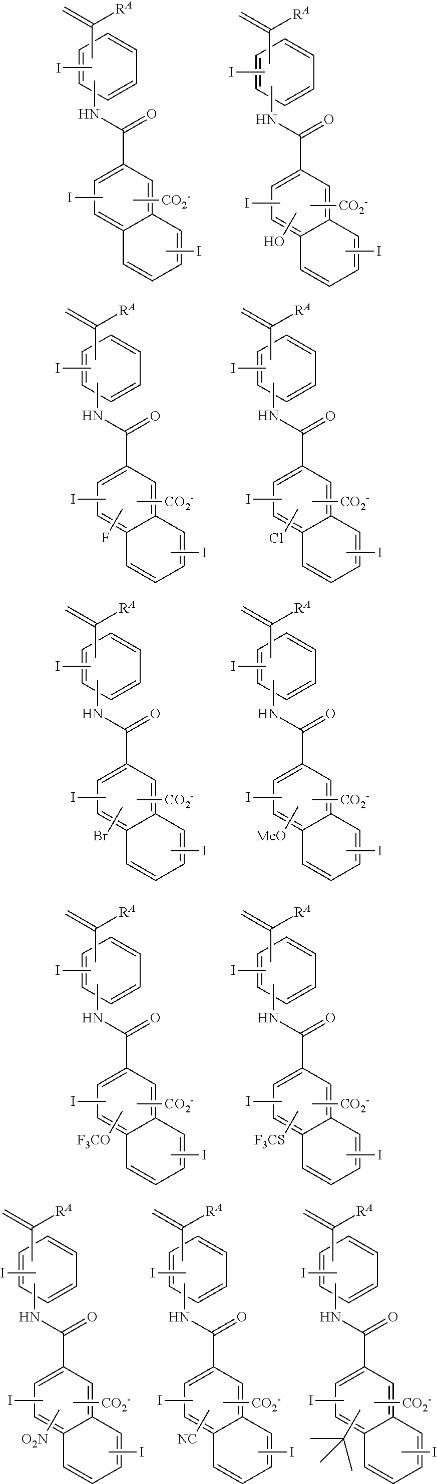

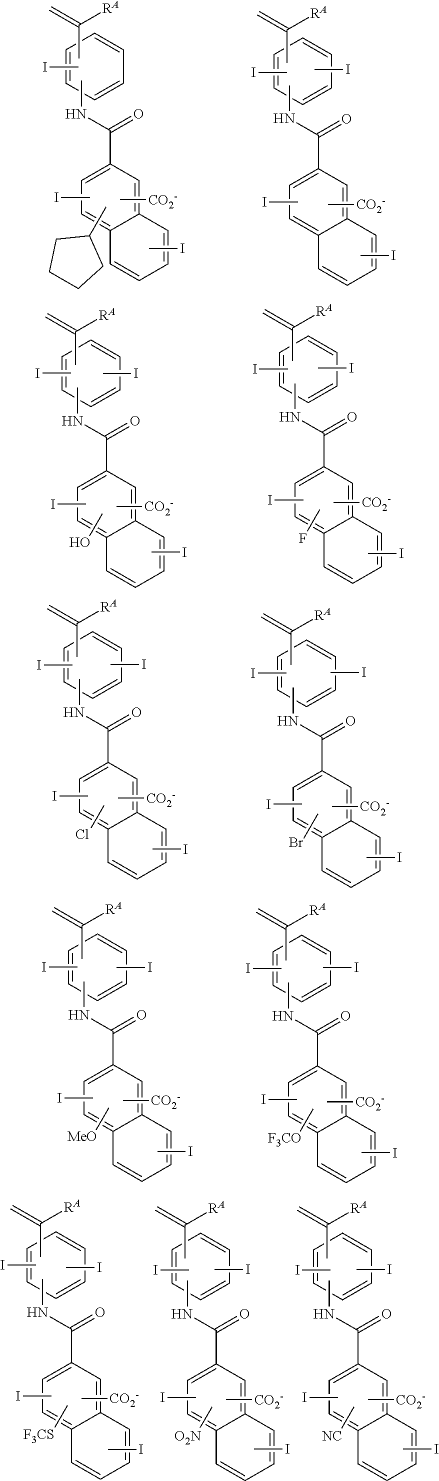

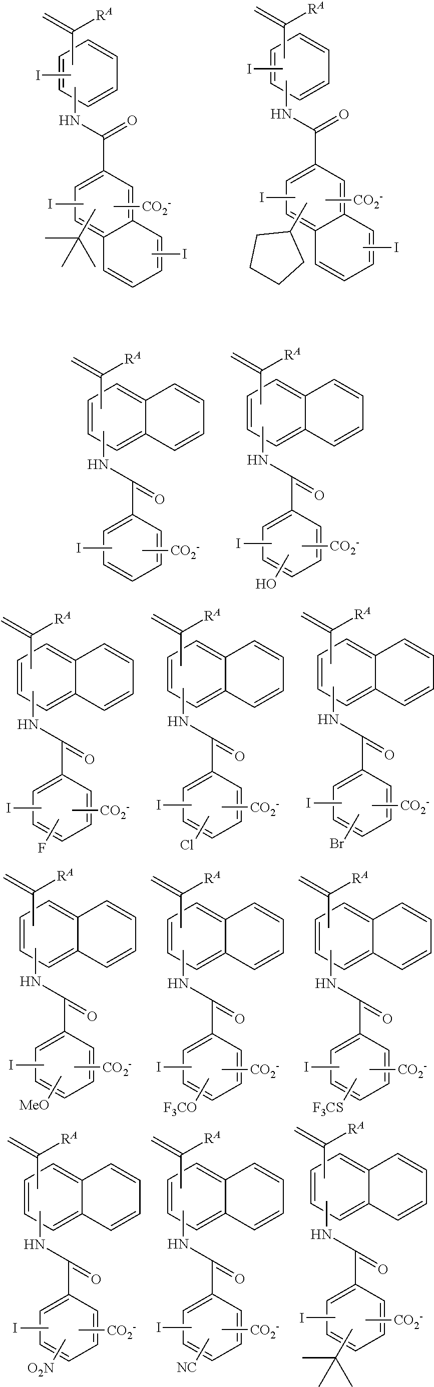

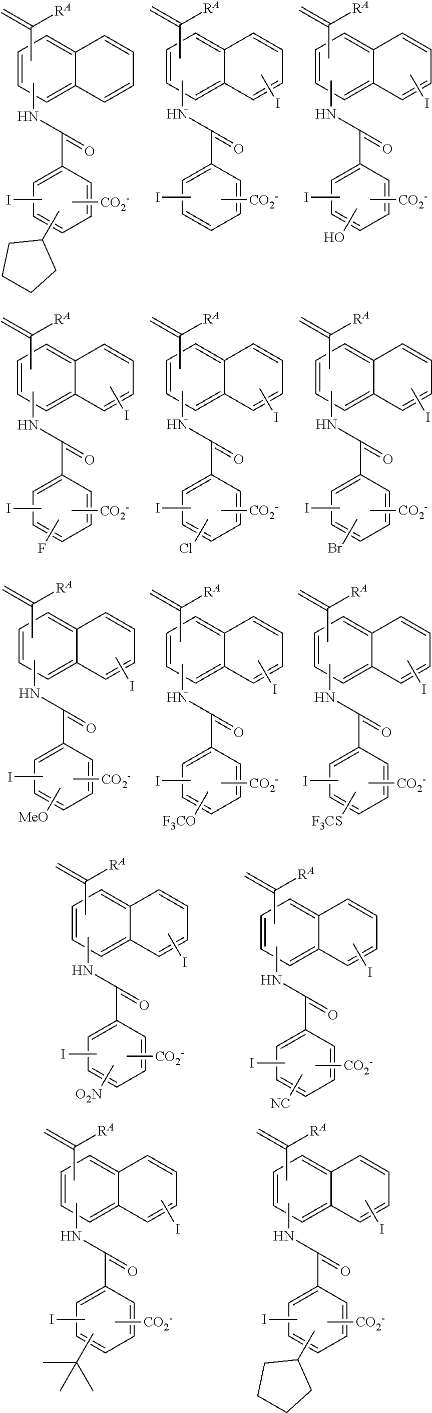

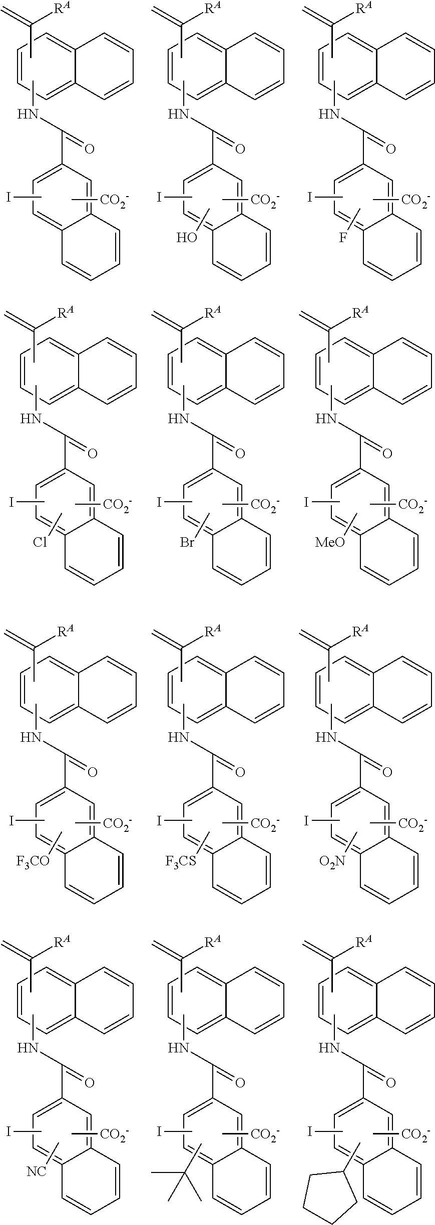

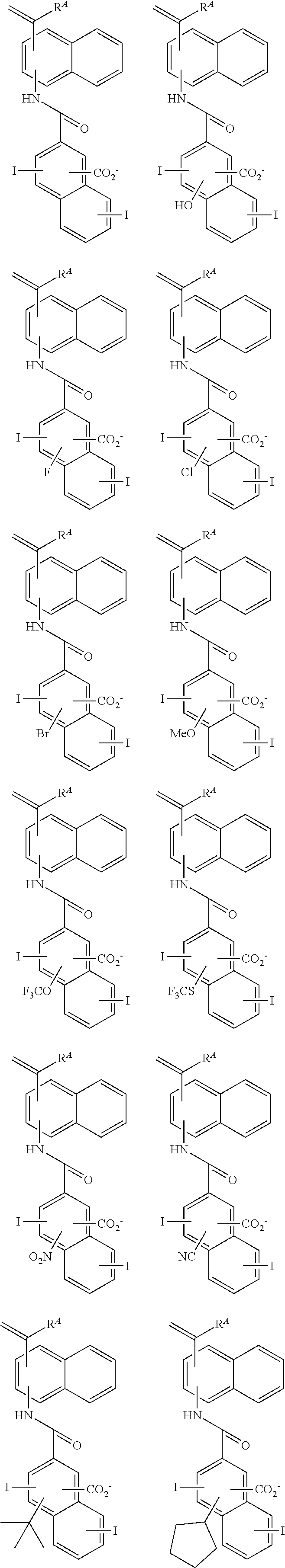

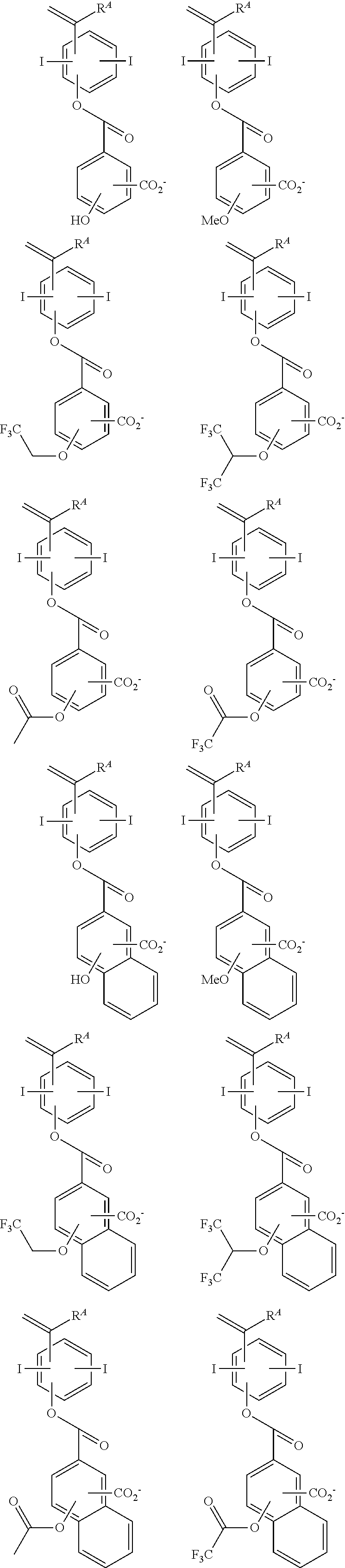

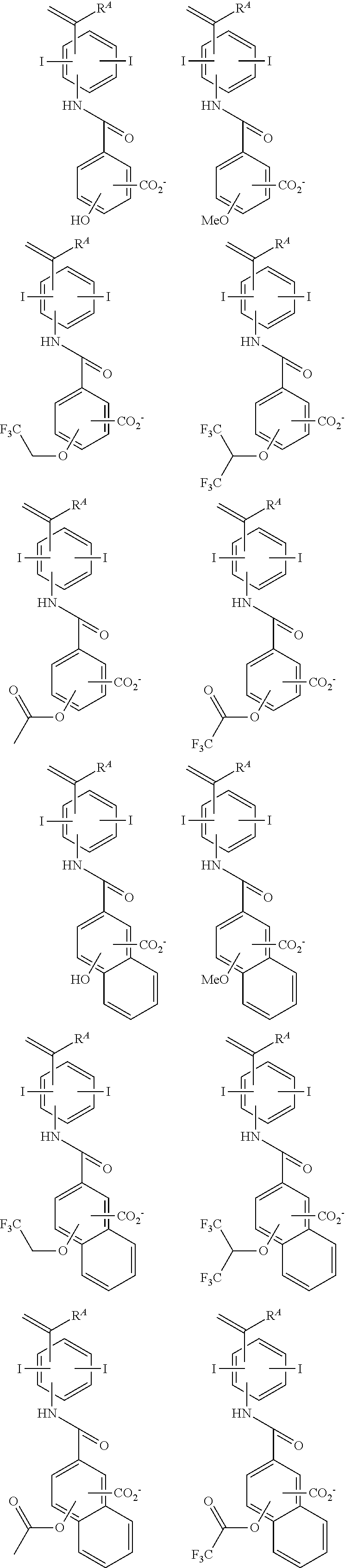

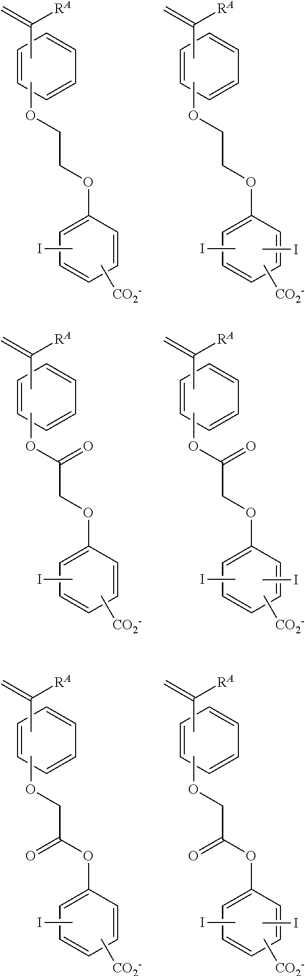

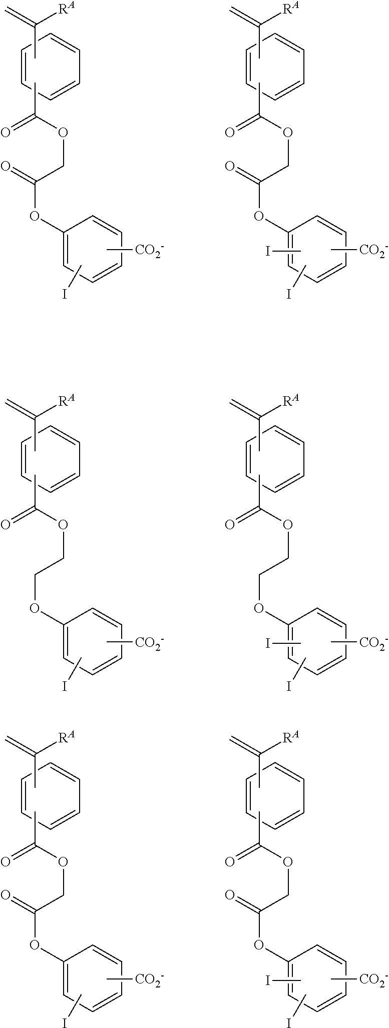

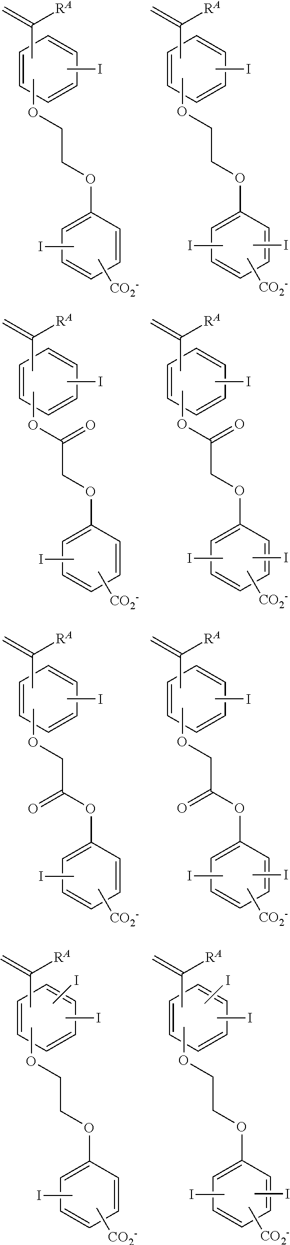

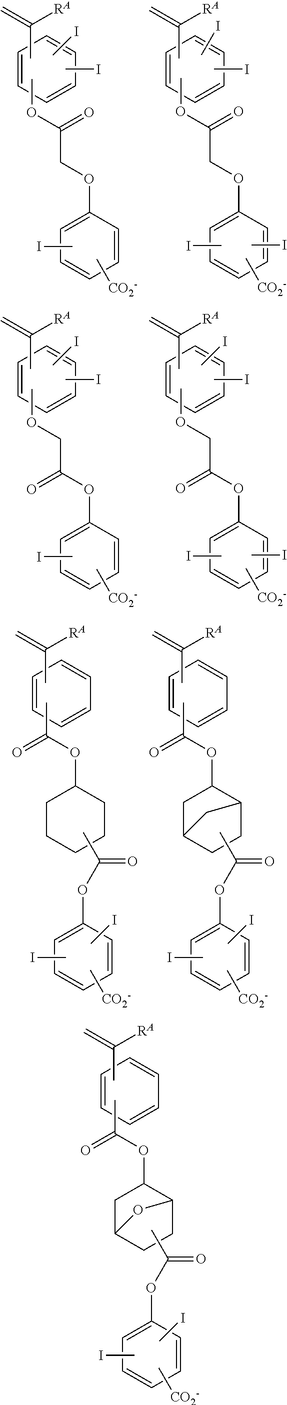

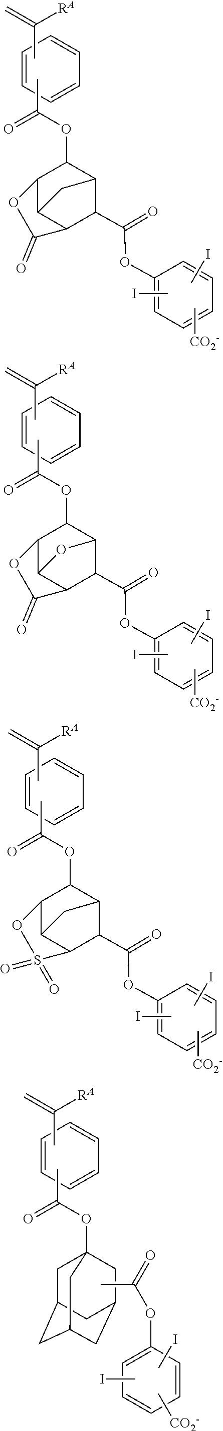

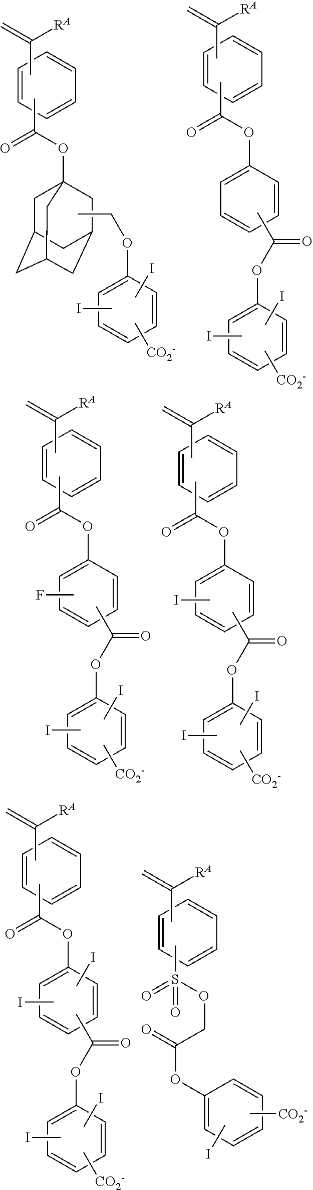

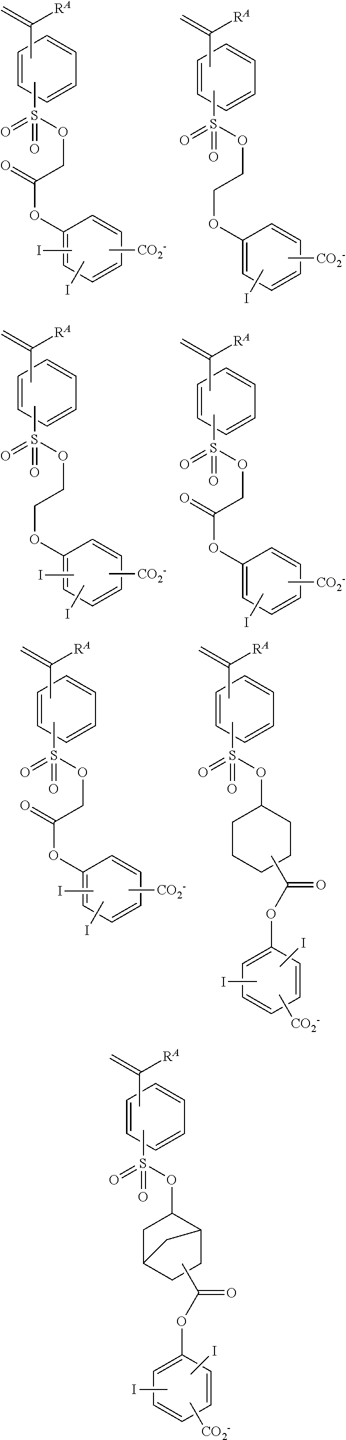

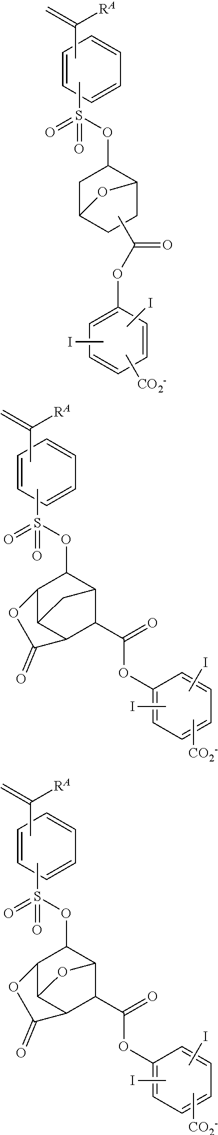

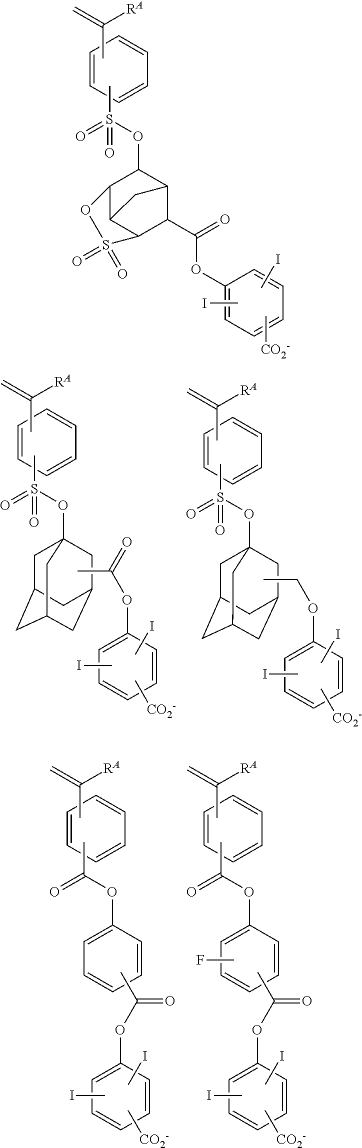

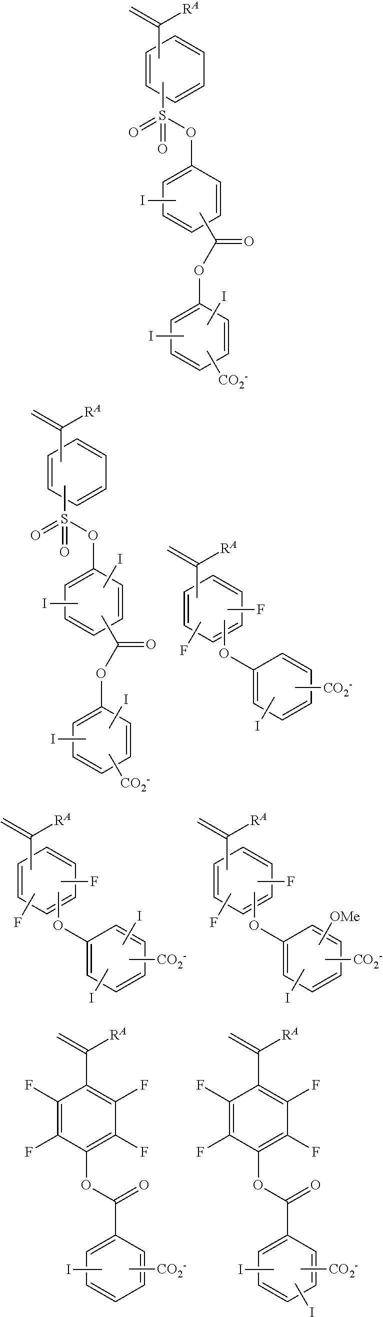

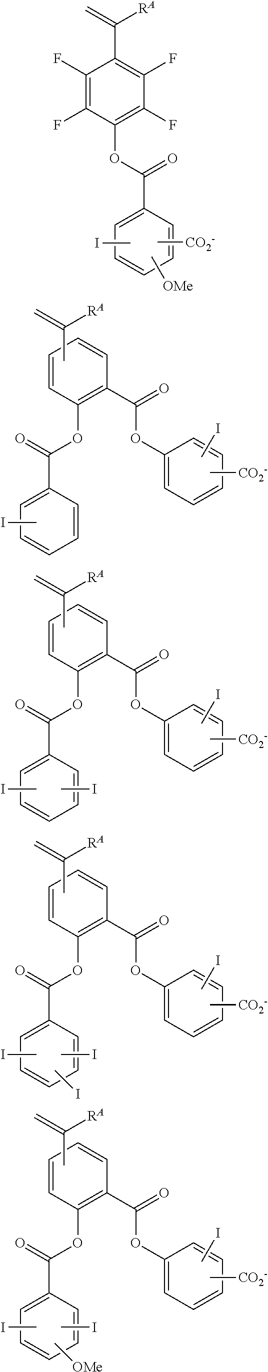

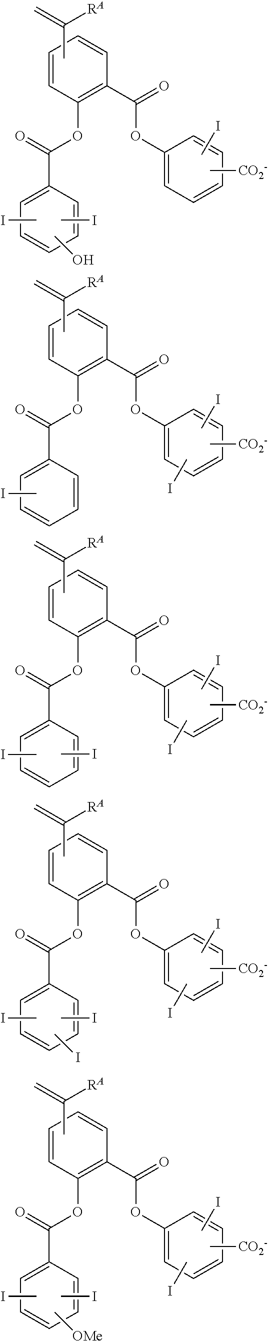

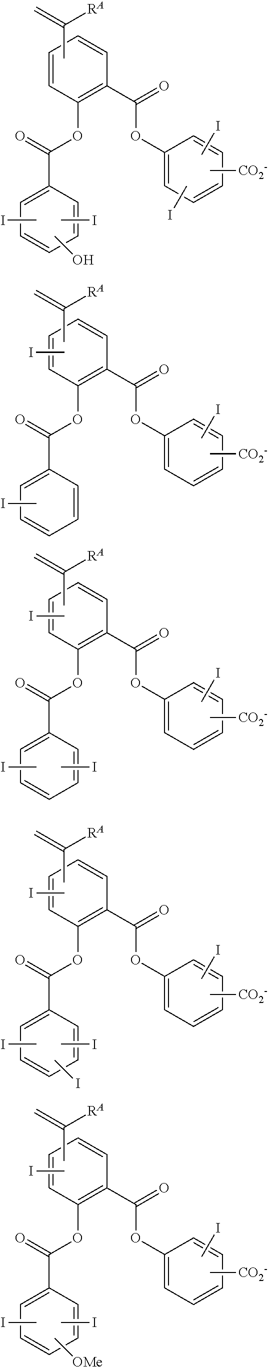

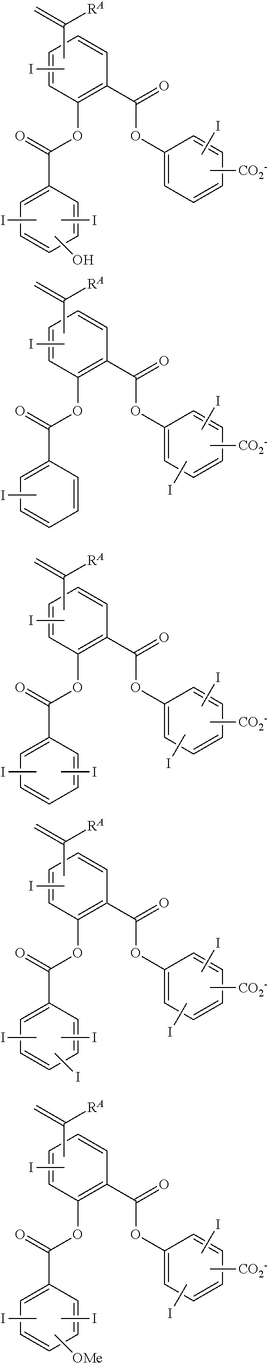

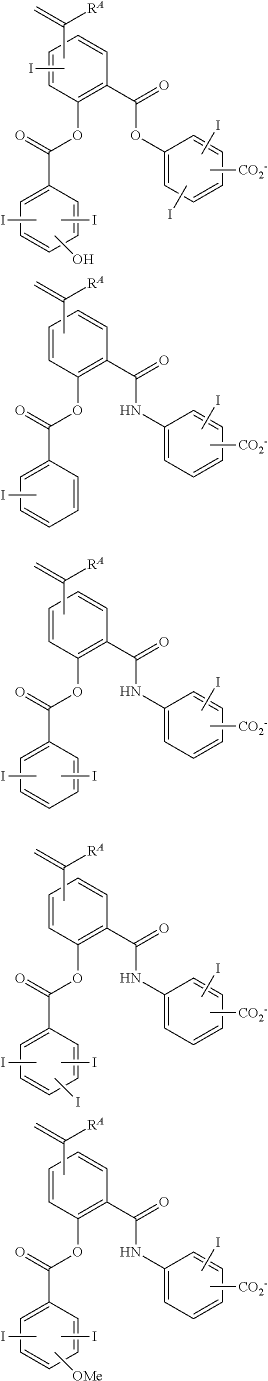

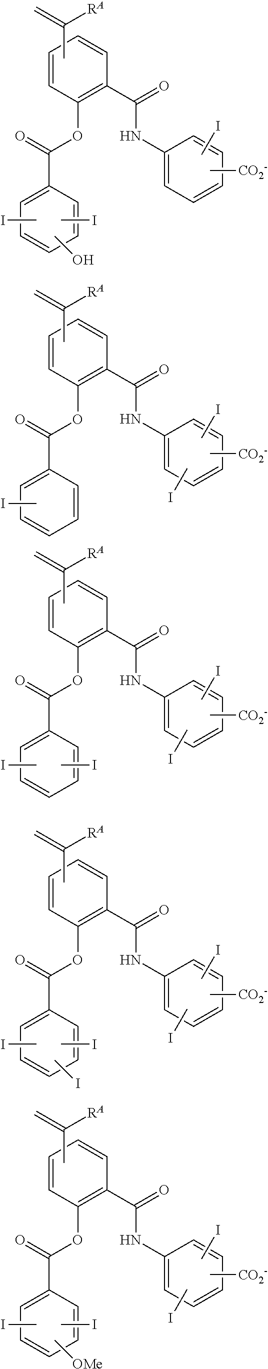

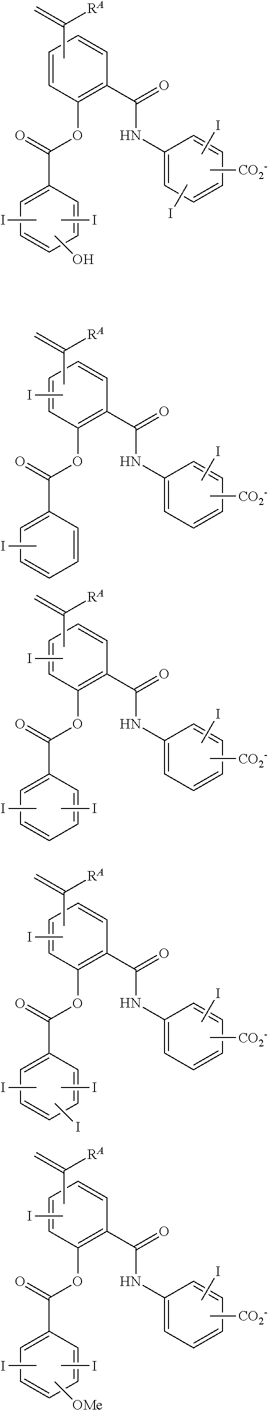

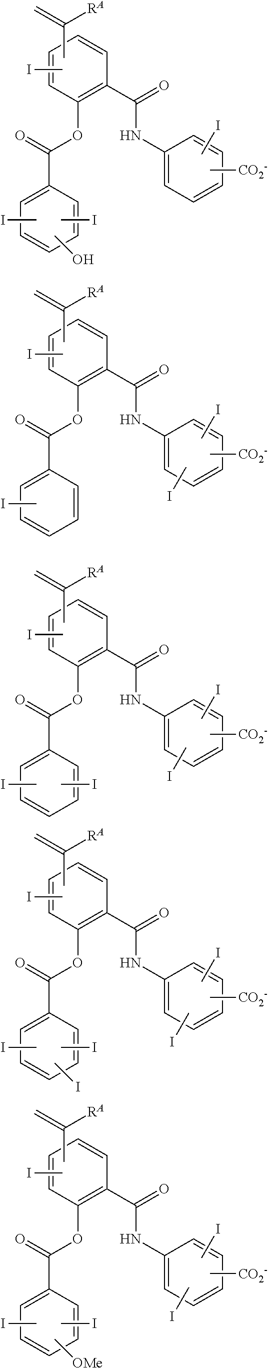

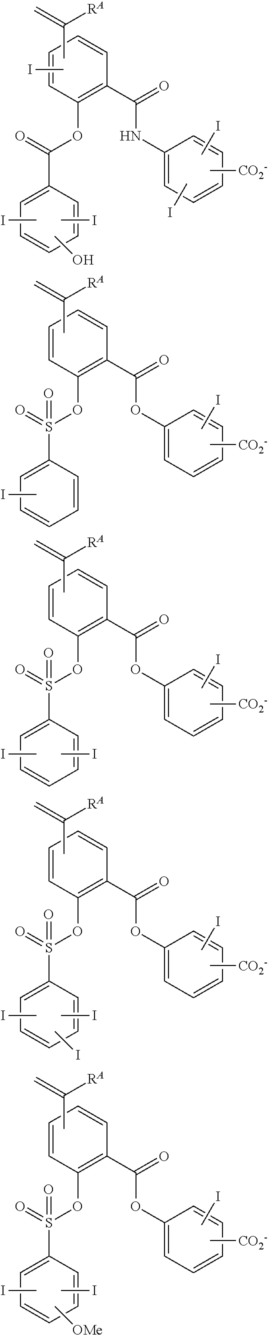

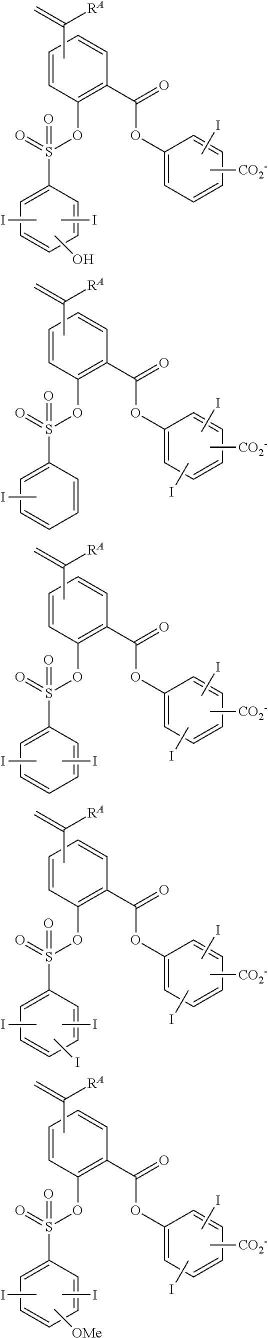

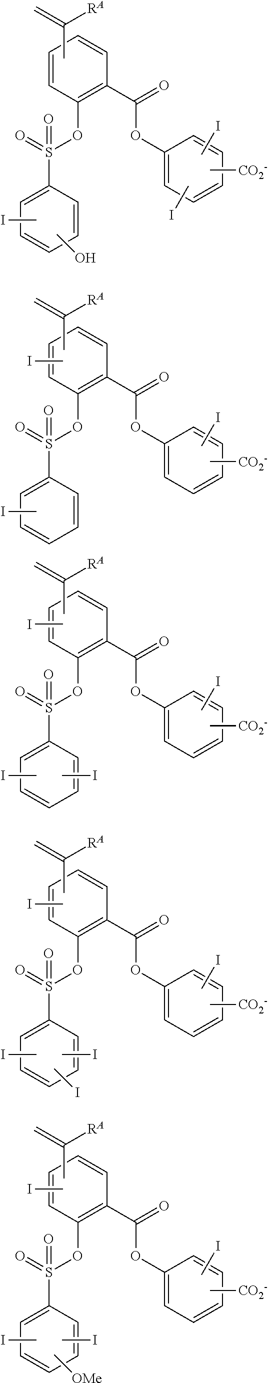

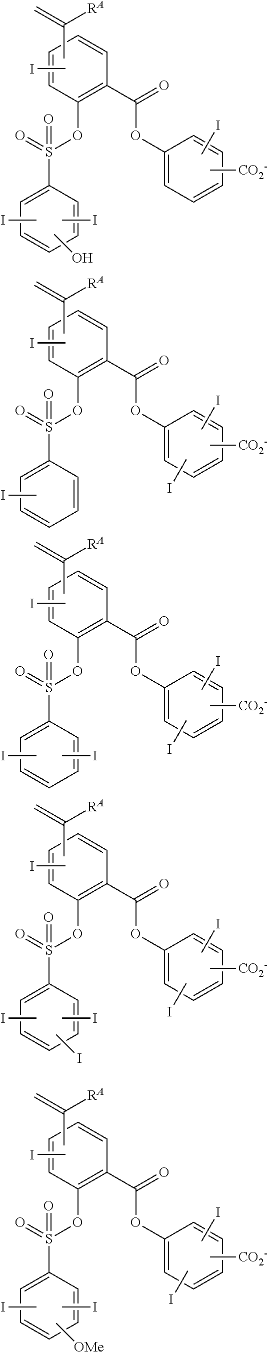

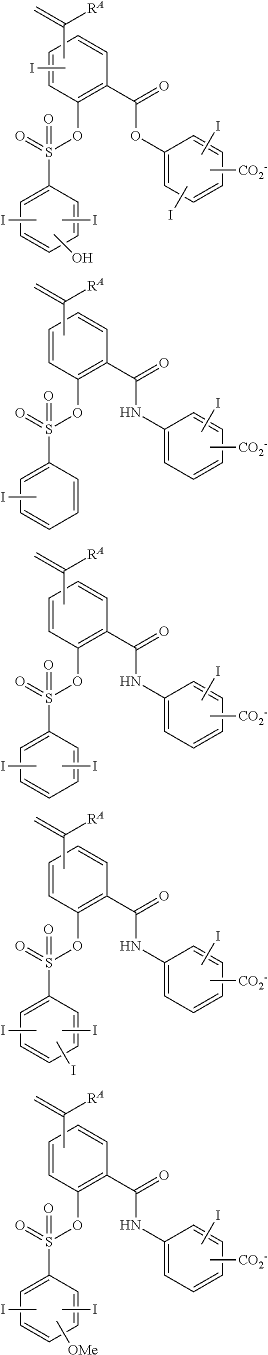

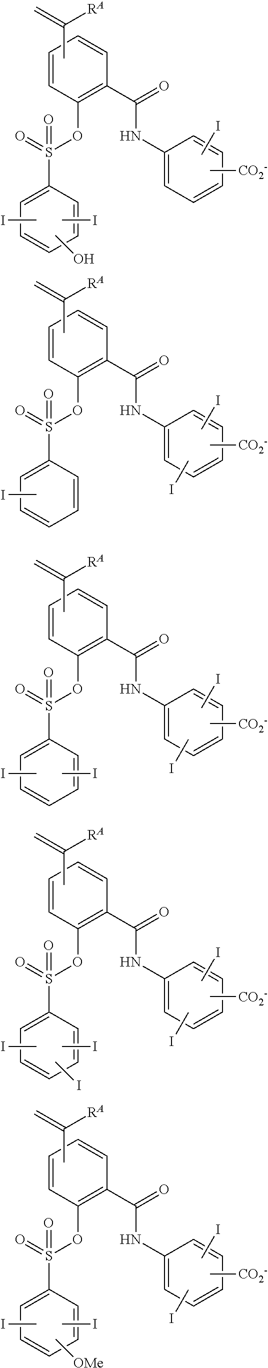

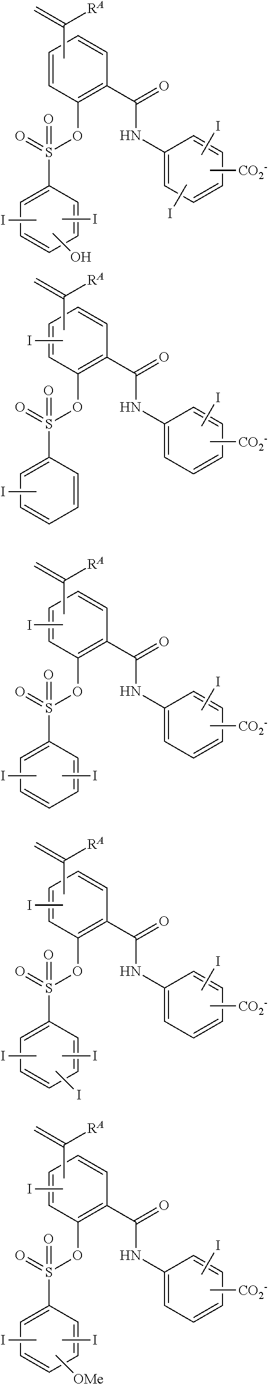

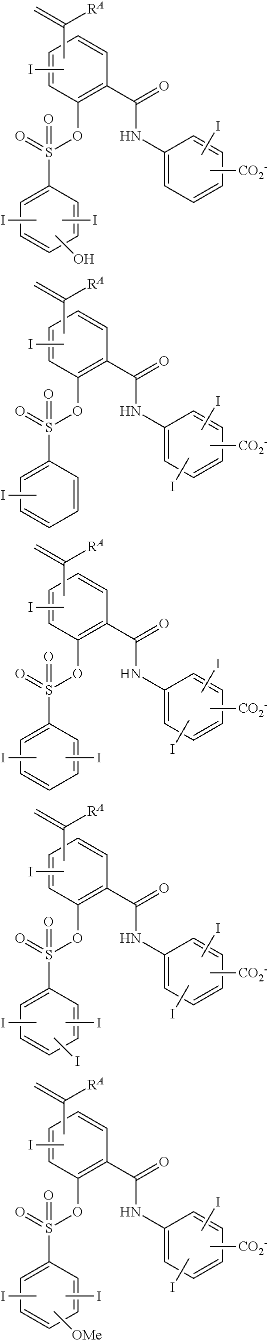

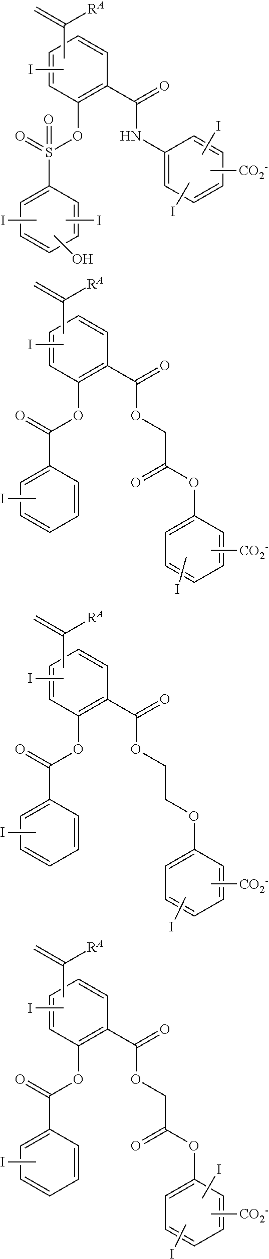

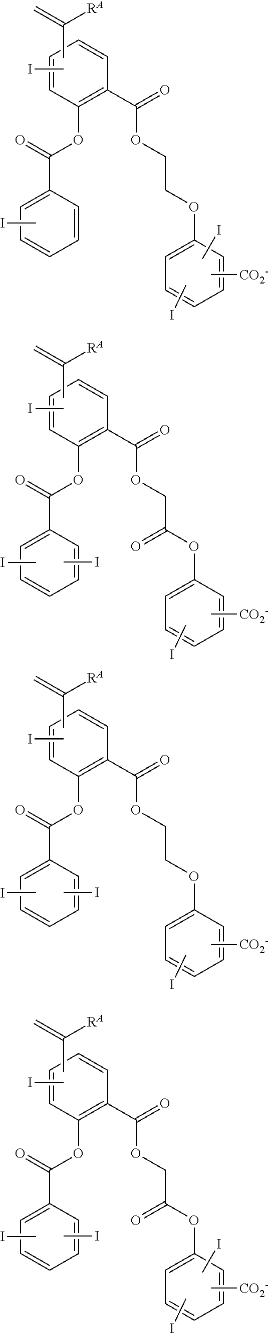

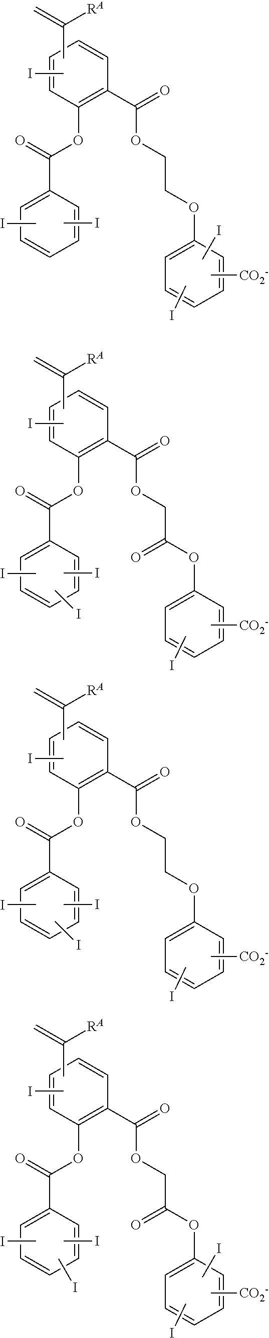

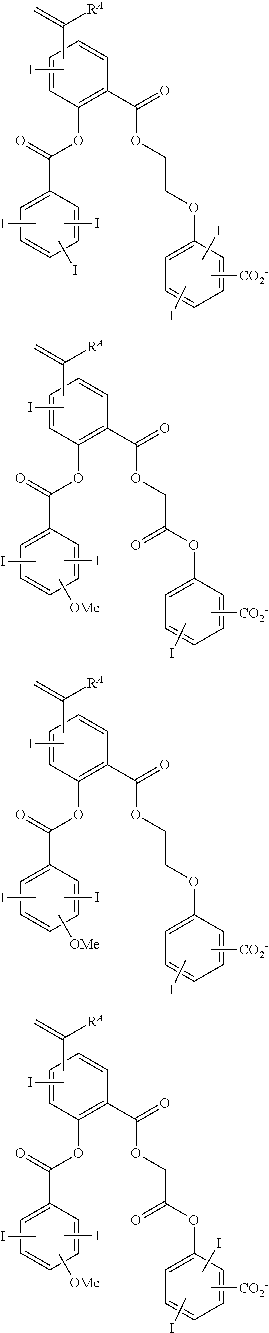

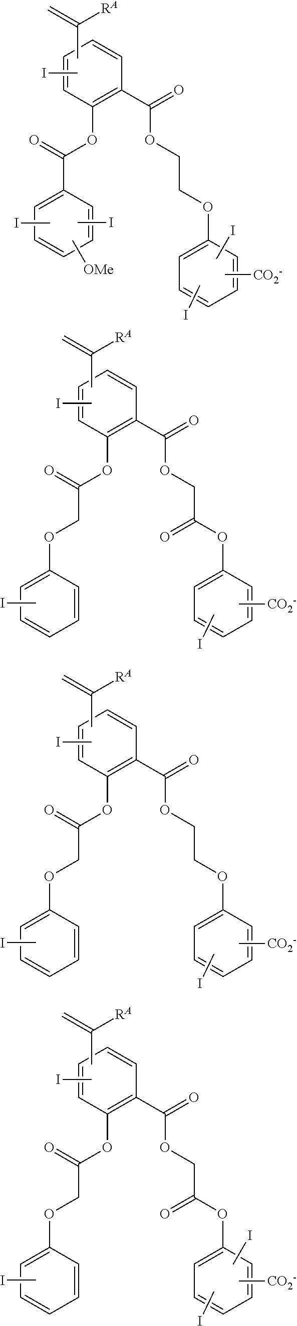

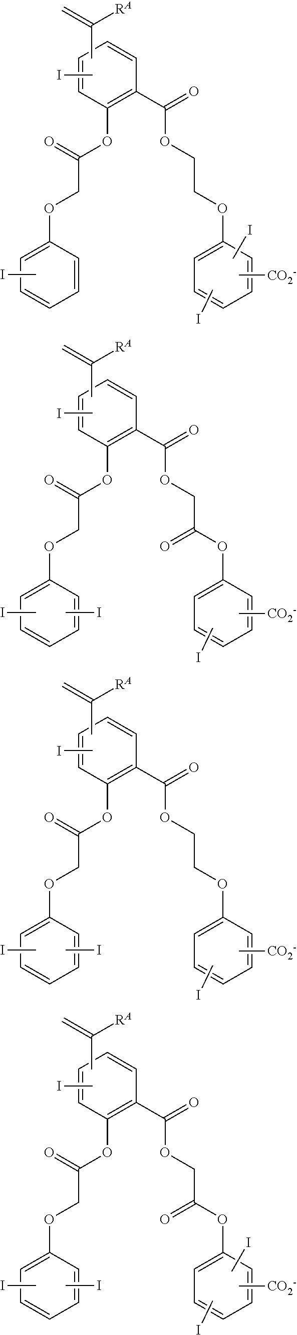

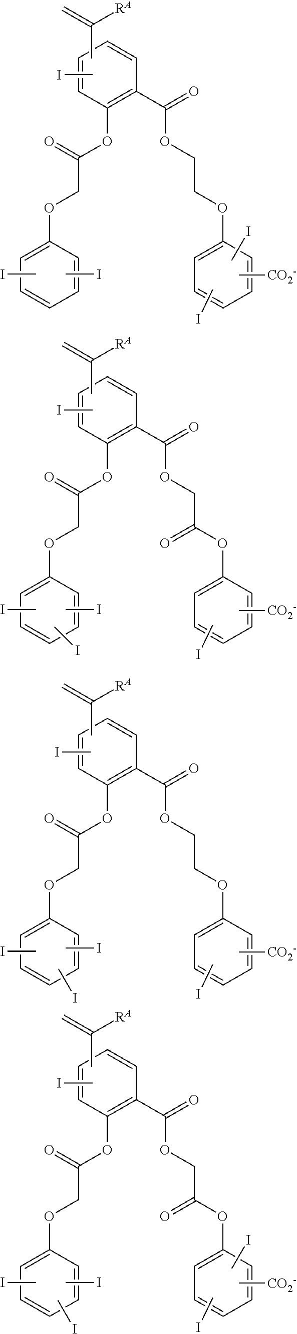

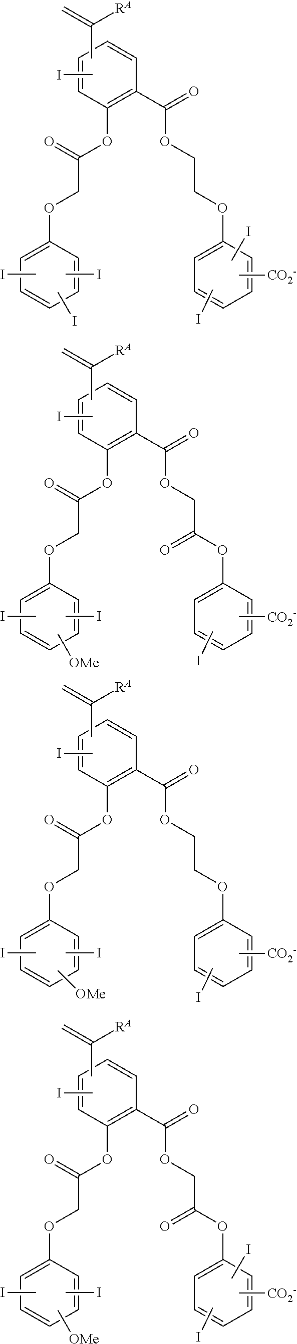

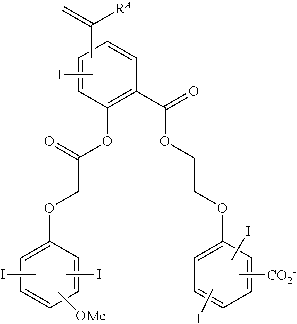

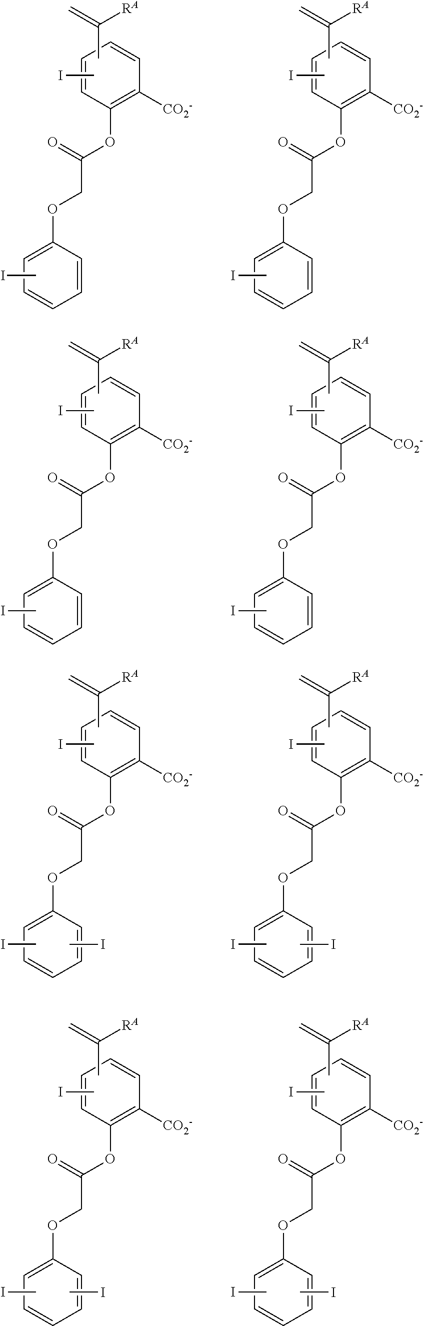

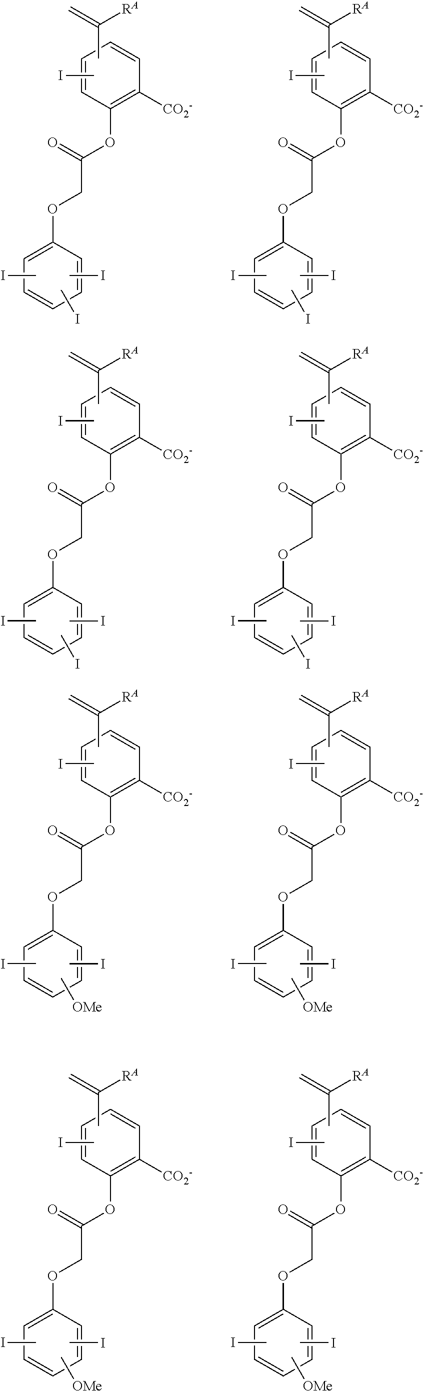

Other specific examples of the onium salt monomer having the formula (a) include, but are not limited to, those described in paragraphs [0049], to [0056], and [0062] to [0064] of JP 7433394 and paragraph [0116] of JP-A 2024-178693.

Specific examples of the sulfonium salt monomer of the present invention include any combination of the anion and the cation described above.

The sulfonium salt monomer of the present invention can be synthesized by a known method. Specific examples thereof include, but are not limited to, methods described in paragraphs [0402] to [0405] of JP 7433394.

### [Polymer]

A polymer of the present invention contains a repeat unit (hereinafter, also referred to as a repeat unit a) derived from the sulfonium salt monomer having the formula (a).

The polymer of the present invention is a polymer-bound quencher that functions as a quencher in a chemically amplified resist composition and also functions as a base polymer. The polymer of the present invention has a structural characteristic of containing a repeat unit having a salt structure containing a carboxylic acid anion having a benzene or naphthalene structure directly bonded to a main chain and having an iodine atom, and a sulfonium cation having a pentafluorosulfanyl group and a hydrocarbyloxycarbonyl group, the repeat unit being derived from the sulfonium salt monomer of the present invention. Since an iodine atom has extremely large absorption of EUV having a wavelength of 13.5 nm, secondary electrons are generated during exposure, and energy of the secondary electrons is transferred to an acid generator to promote decomposition, thereby increasing sensitivity. The polymerizable group having a styrene or vinylnaphthalene structure is more rigid than a polymerizable group such as a methacrylate and improves a glass transition temperature (Tg) of the polymer. It is considered that aromatic rings in the polymer or between the polymers interact with each other (π-π stacking effect), whereby the polymers are regularly arranged, and resistance to pattern collapse is exhibited against a developer even during fine pattern formation. Also in an etching step after fine pattern formation, excellent etching resistance is exhibited by having an aromatic ring directly connected to the main chain. In addition, a pentafluorosulfanyl group is a strong electron-withdrawing group, and is bonded to an aromatic ring of a triarylsulfonium cation as a substituent, whereby LUMO in the frontier molecule activation theory is lowered, and the sulfonium cation easily receives secondary electrons, which promotes decomposition of the sulfonium cation. The acid-unstable group having a tertiary/secondary ether structure, a tertiary/secondary carbonate structure, or an acetal structure, the acid-unstable group being bonded to an aromatic ring of the sulfonium cation as a substituent, undergoes a deprotection reaction with an acid generated by exposure to generate a corresponding phenol. By change of the structure from a fat-soluble structure to a hydrophilic structure, contrast between an exposed portion and an unexposed portion is improved. In addition, a pentafluorosulfanyl group is bonded to a carbon atom adjacent to a carbon atom to which a phenolic hydroxy group to be generated is bonded, whereby acidity of the phenol is improved due to an electron withdrawing property of the pentafluorosulfanyl group. When an exposed portion in which the deprotection reaction has progressed is developed with an alkaline developer, the phenol having an improved acidity has improved solubility in the alkaline developer, whereby development residues can be suppressed, and collapse of a resist pattern can be suppressed by suppression of swelling by the alkaline developer. On the other hand, the carboxylic acid anion has relatively high basicity, and can effectively trap an acid generated from a strong acid. By this synergistic effect, lithographic performance such as LWR or CDU is improved while the polymer has high sensitivity, and pattern formation resistant to pattern collapse is possible. Therefore, the polymer is suitable particularly as a material of a chemically amplified positive resist composition.

The polymer may contain a repeat unit having the following formula (b1) (hereinafter, also referred to as a repeat unit b1) or a repeat unit having the following formula (b2) (hereinafter, also referred to as a repeat unit b2).

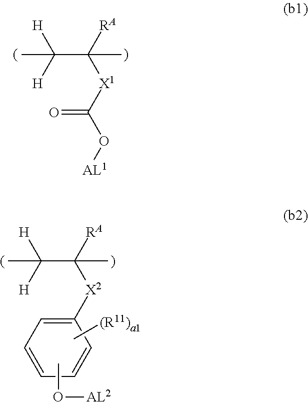

In the formulas (b1) and (b2), R^{A}s are each independently a hydrogen atom, a fluorine atom, a methyl group, or a trifluoromethyl group.

In the formula (b1), X¹ is a single bond, a phenylene group, a naphthylene group, *—C(—O)—O—X¹¹—, or *—C(═O)—N(H)—X¹¹—, and the phenylene group or the naphthylene group may be substituted with a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom. X¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring. * represents a bond with a carbon atom of a main chain.

In the formula (b2), X² is a single bond, *—C(═O)—O—, or *—C(═O)—N(H)—. * represents a bond with a carbon atom of a main chain. R¹¹ is a halogen atom, a cyano group, a hydroxy group, a nitro group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a1 is 0, 1, 2, 3, or 4, and preferably 0 or 1. When a1 is 2, 3, or 4, R¹¹s may be the same as or different from each other.

In the formulas (b1) and (b2), AL¹ and AL² are each independently an acid-unstable group. Specific examples of the acid-unstable group include those described in JP-A 2013-80033 and JP-A 2013-83821.

Typically, specific examples of the acid-unstable group include those having the following formulas (AL-3) to (AL-5):

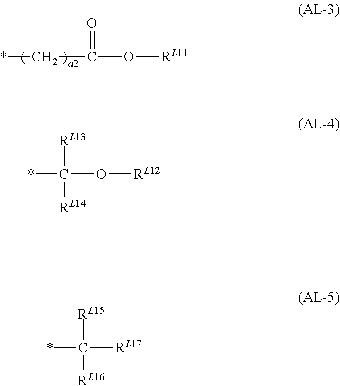

wherein * is a bond.

In the formulas (AL-3) and (AL-4), R^{L11} and R^{L12} are each independently a C₁-C₄₀ hydrocarbyl group, and may contain a heteroatom such as an oxygen atom, a sulfur atom, a nitrogen atom, a fluorine atom, or an iodine atom. The hydrocarbyl group may be saturated or unsaturated, and may be linear, branched, or cyclic. The hydrocarbyl group preferably has 1 to 20 carbon atoms.

In the formula (AL-3), a2 is an integer of 0 to 10, and preferably 1, 2, 3, 4 or 5. In the formula (AL-4), R^{L13} and R^{L14} are each independently a hydrogen atom or a C₁-C₂₀ hydrocarbyl group, and may contain a heteroatom such as an oxygen atom, a sulfur atom, a nitrogen atom, a fluorine atom, or an iodine atom. The hydrocarbyl group may be saturated or unsaturated, and may be linear, branched, or cyclic. Any two of R^{L12}, R^{L13}, and R^{L14} may be bonded to each other to form a C₃-C₂₀ ring together with a carbon atom to which they are bonded or a carbon atom and an oxygen atom. The ring is preferably a C₄-C₁₆ ring, and particularly preferably an alicyclic ring.

In the formula (AL-5), R^{L15}, RL16, and R^{L17} are each independently a C₁-C₂₀ hydrocarbyl group, and may contain a heteroatom such as an oxygen atom, a sulfur atom, a nitrogen atom, a fluorine atom, or an iodine atom. The hydrocarbyl group may be saturated or unsaturated, and may be linear, branched, or cyclic. Any two of R^{L15}, RL16, and R^{L17} may be bonded to each other to form a C₃-C₂₀ ring together with a carbon atom to which they are bonded. The ring is preferably a C₄-C₁₆ ring, and particularly preferably an alicyclic ring.

Other specific examples of the acid-unstable group include those described in paragraphs [0064] to [0068] of JP-A 2023-123222 and those described in paragraphs [0013] and [0014] of JP 7492842. In these, generation of a conjugated olefin or an acrylic acid ester derivative after an acid elimination reaction is used as a driving force for progress of the reaction.

Specific examples of the repeat unit b1 include, but are not limited to, those listed below. Note that, in the following formulas, R^{A} and AL¹ are as defined above.

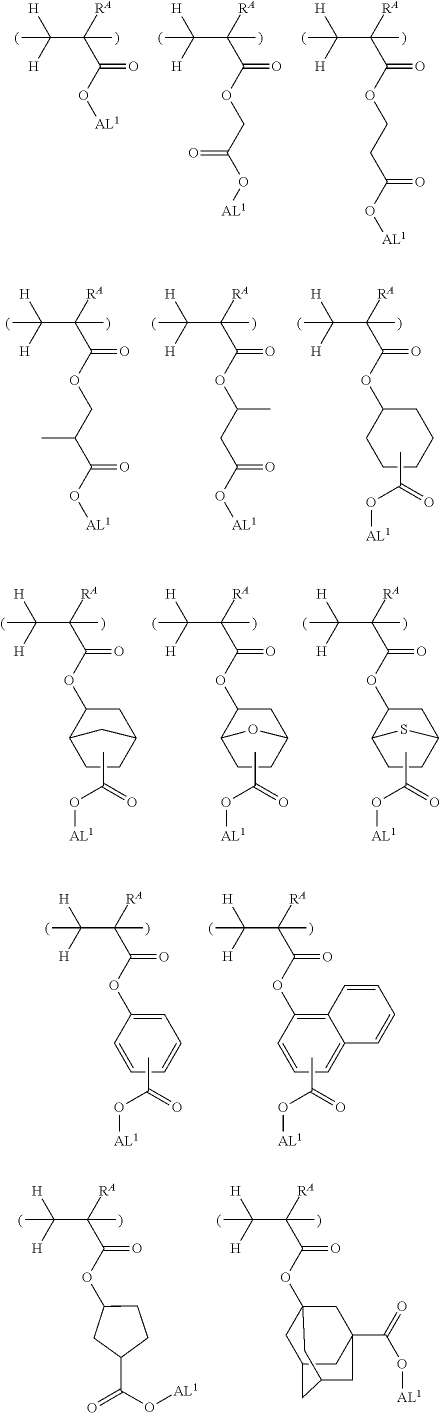

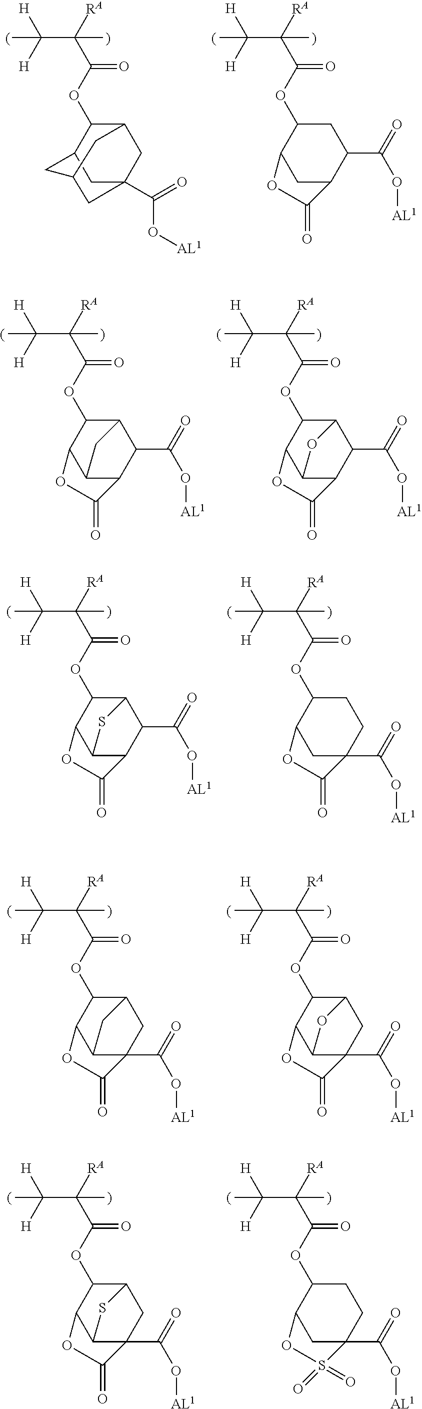

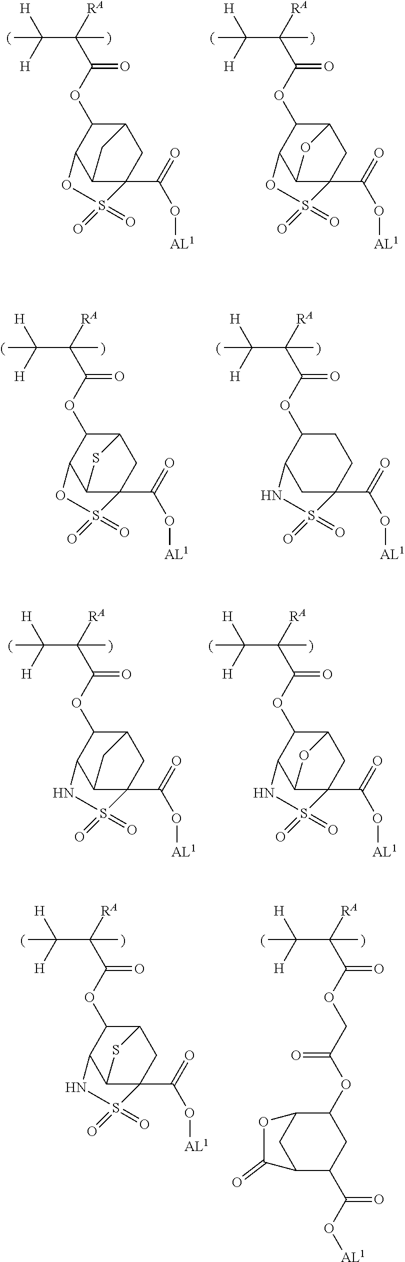

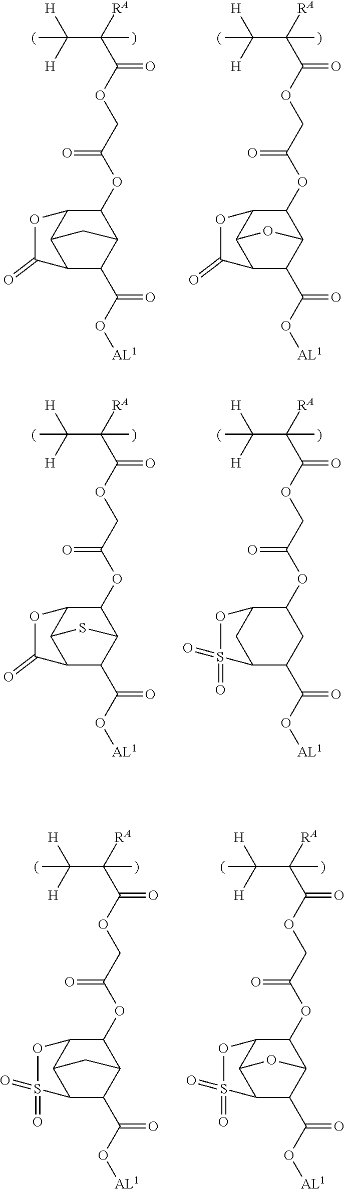

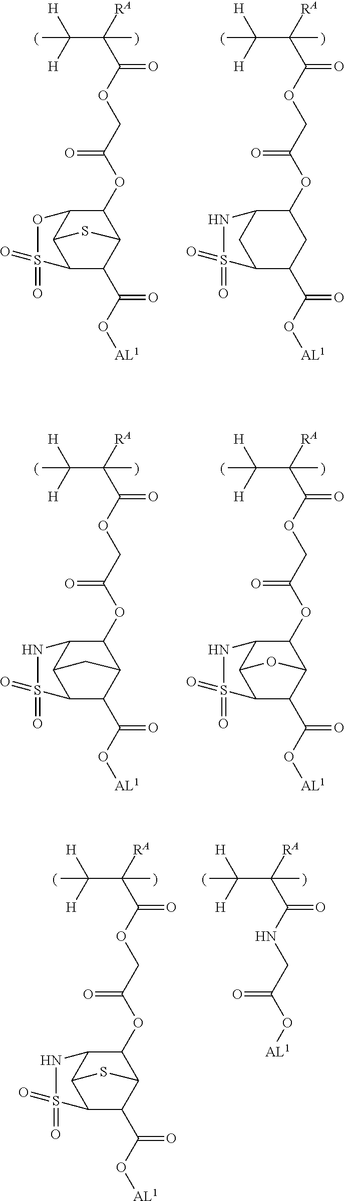

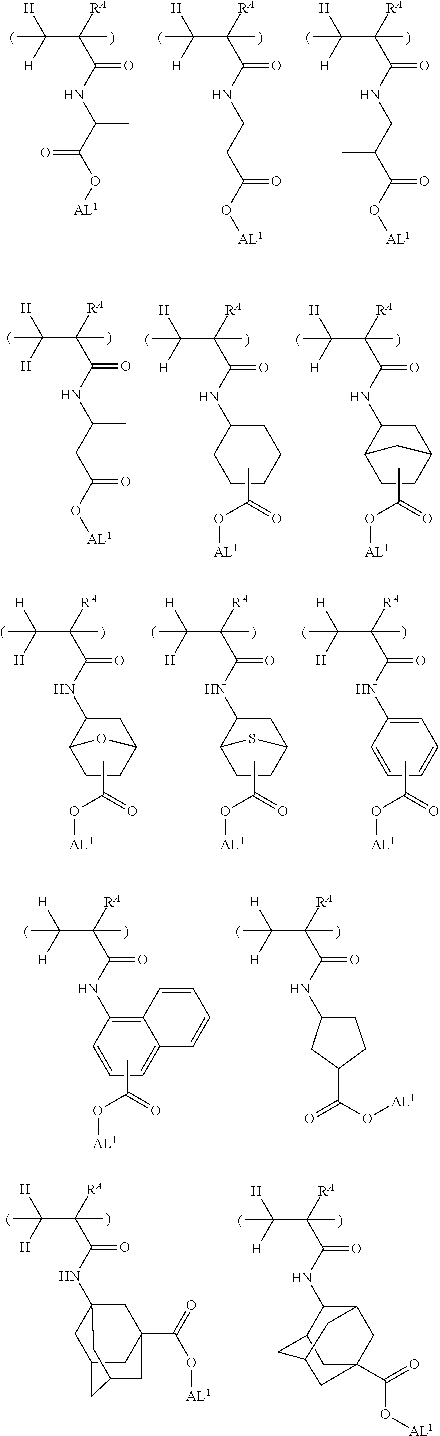

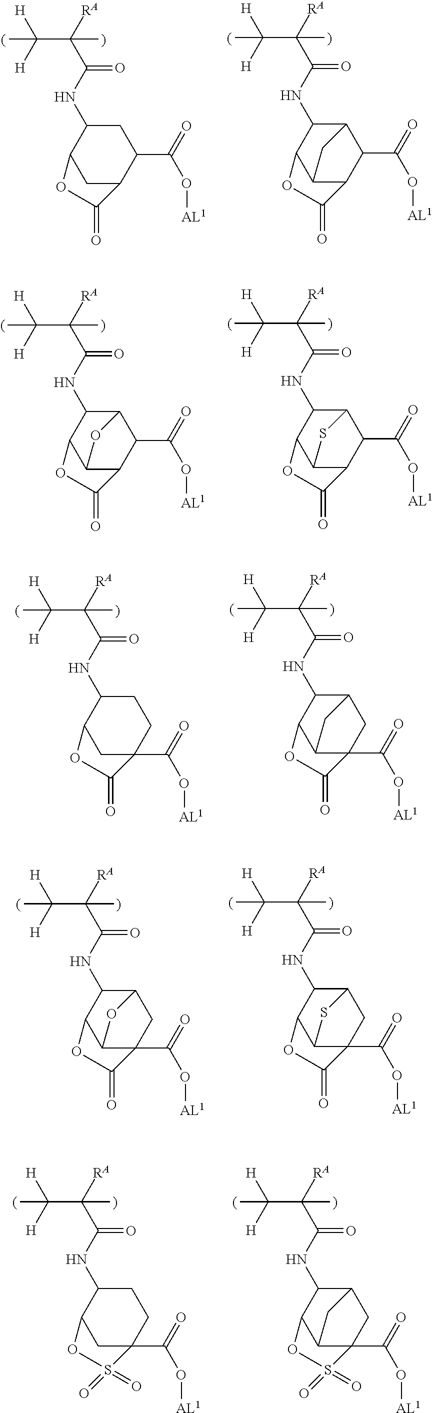

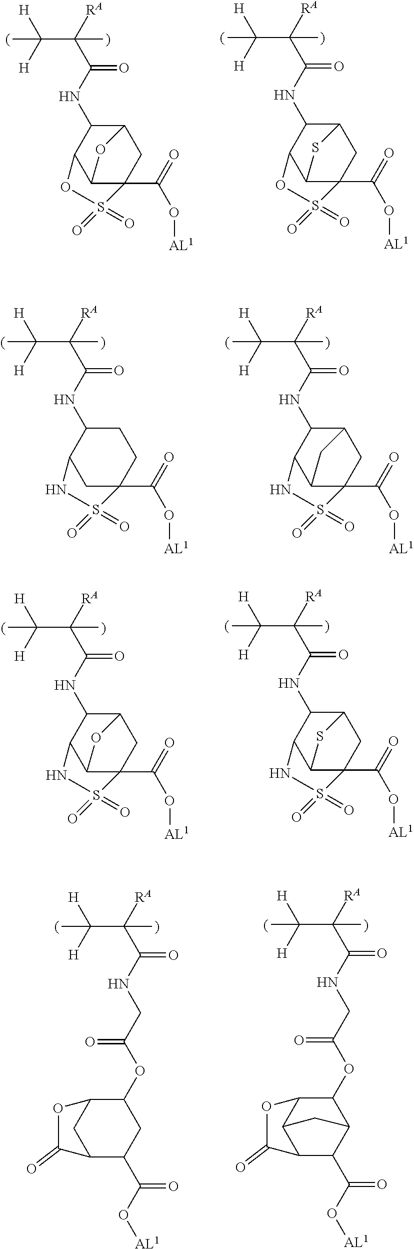

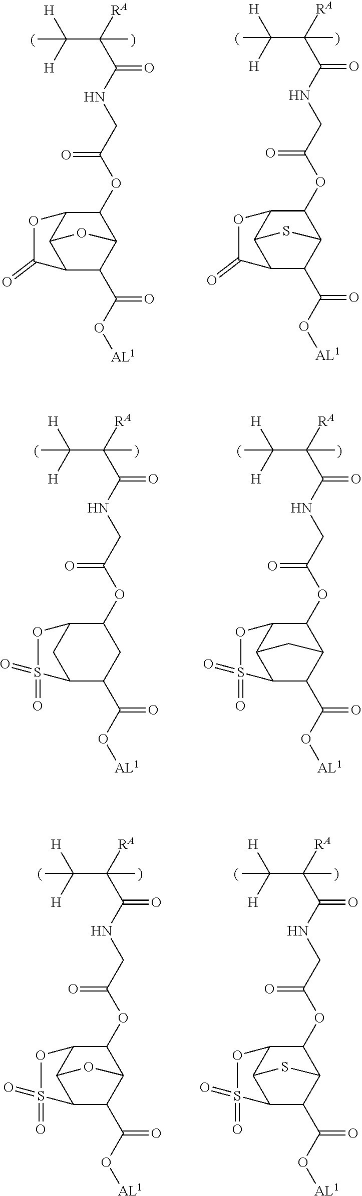

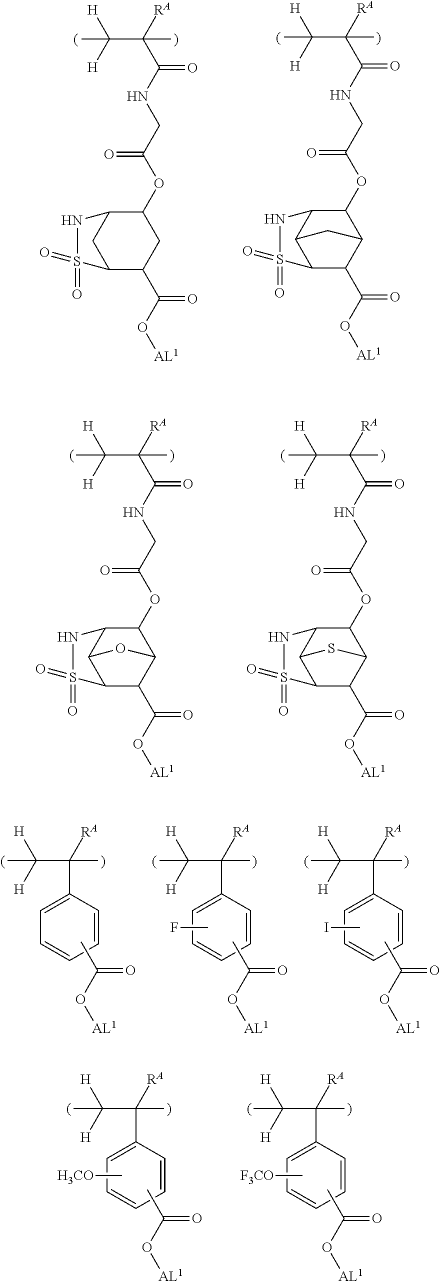

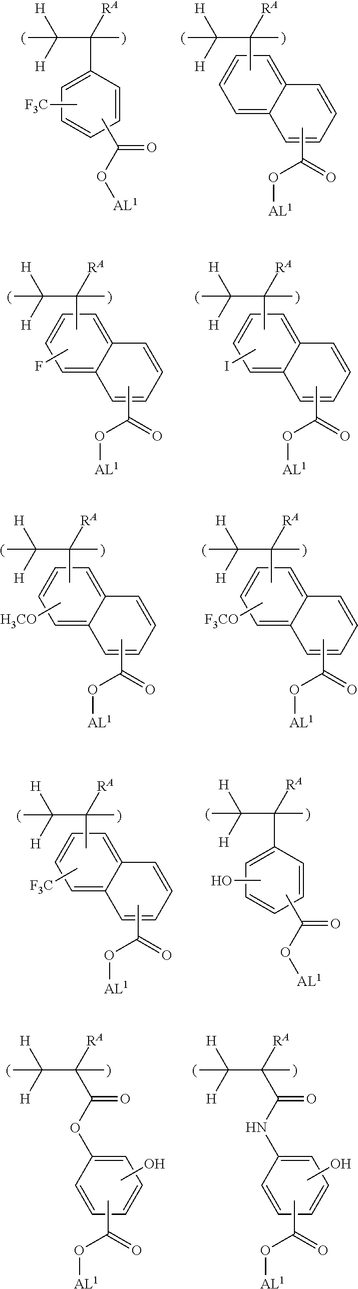

Specific examples of the repeat unit b2 include, but are not limited to, those listed below. Note that, in the following formulas, R^{A} and AL² are as defined above.

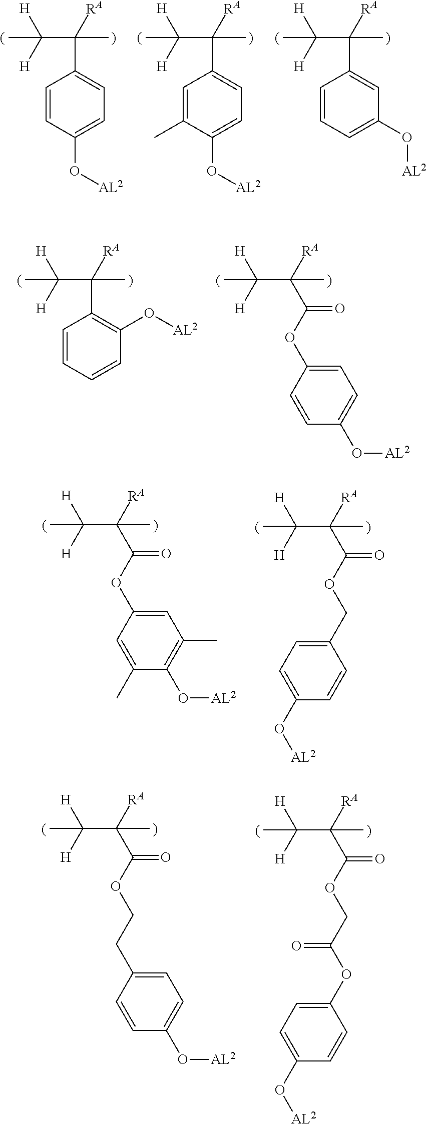

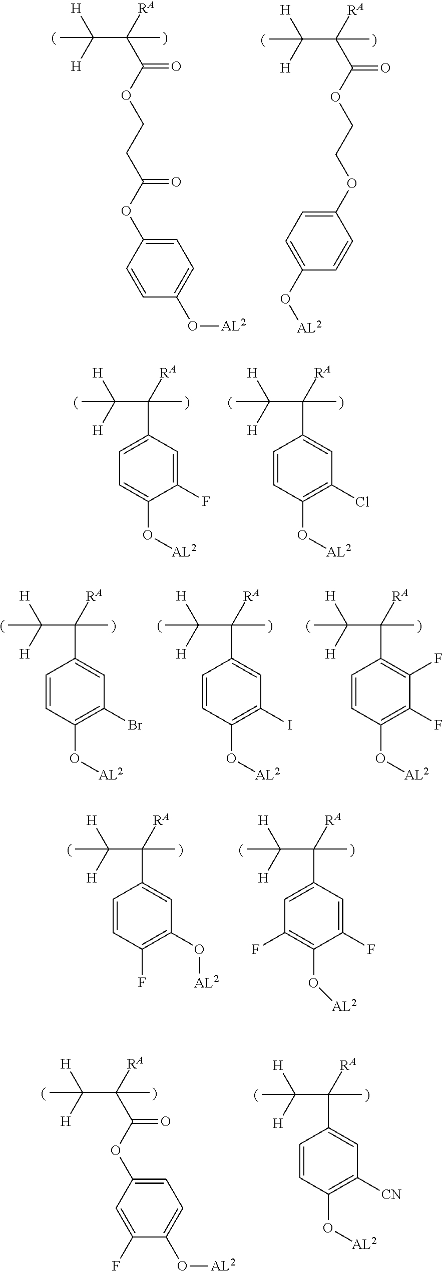

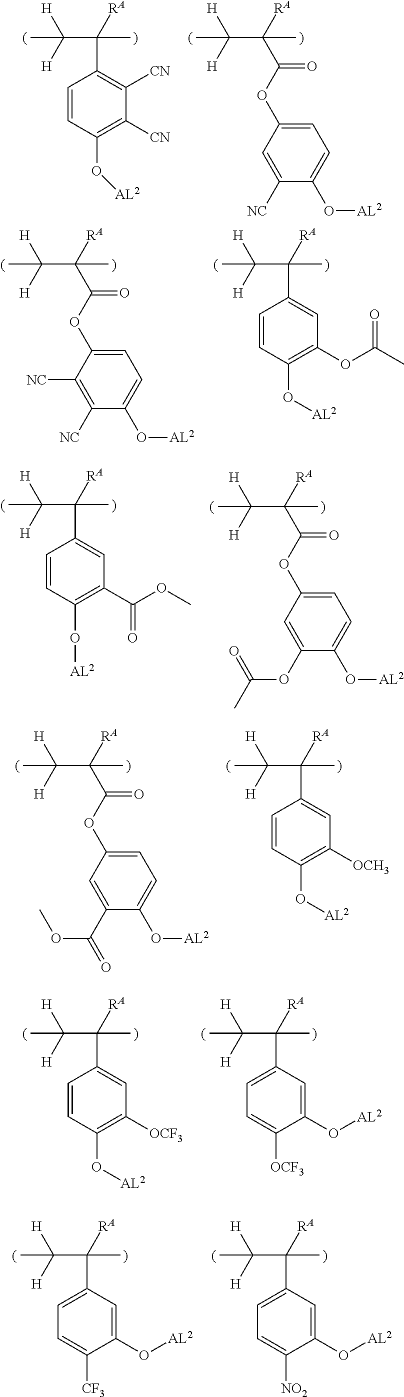

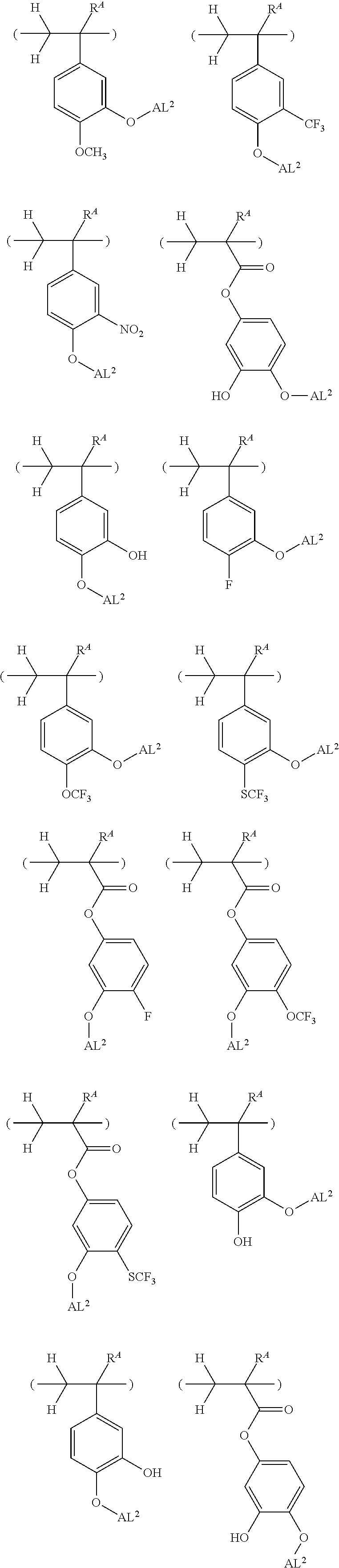

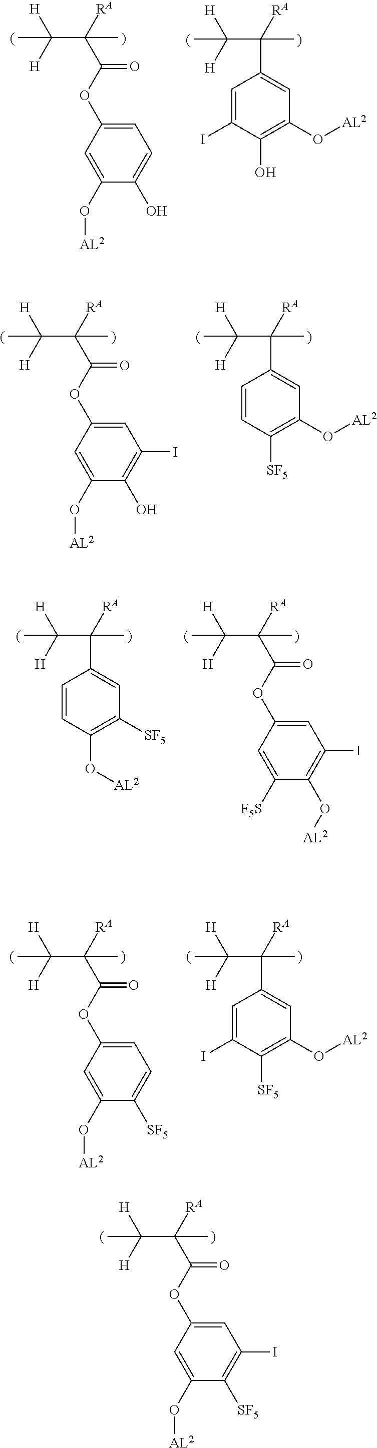

The polymer may contain a repeat unit having the following formula (b3) (hereinafter, also referred to as a repeat unit b3).

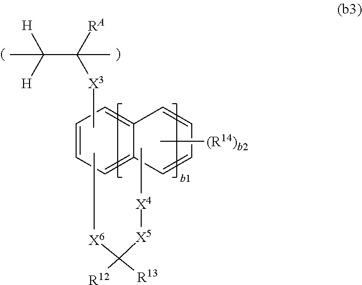

In the formula (b3), b1 is 0 or 1. When b1 is 0, it is a benzene ring, and when b1 is 1, it is a naphthalene ring, but a benzene ring in which b1 is 0 is preferable from a viewpoint of solvent solubility. When b1 is 0, b2 is 0, 1, 2, or 3, and when b1 is 1, b2 is 0, 1, 2, 3, 4, or 5. b2 is preferably 0, 1, 2, or 3, and more preferably 0, 1, or 2 from a viewpoint of raw material procurement.

In the formula (b3), R^{A} is a hydrogen atom, a fluorine atom, a methyl group, or a trifluoromethyl group. R^{A} is preferably a hydrogen atom or a methyl group, and more preferably a hydrogen atom.

In the formula (b3), X³ is a single bond, *—C(═O)—O—, or *—C(═O)—N(H)—. * represents a bond with a carbon atom of a main chain, Among these, X³ is preferably a single bond or *—C(═O)—O—, and more preferably a single bond.

In the formula (b3), X⁴ is a single bond, a C₁-C₄ aliphatic hydrocarbylene group, a carbonyl group, a sulfonyl group, or a group obtained by combining these groups. Among these, X⁴ is preferably a single bond, a carbonyl group, or a sulfonyl group from a viewpoint of raw material procurement, and is more preferably a single bond or a carbonyl group from a viewpoint of a polar group generated after reaction.

In the formula (b3), X⁵ and X⁶ are each independently an oxygen atom or a sulfur atom. Provided that X⁴ and X⁶ are bonded to adjacent aromatic ring carbon atoms. X⁵ and X⁶ may be the same as or different from each other, but X⁵ and X⁶ are both preferably oxygen atoms from a viewpoint of reactivity.

In the formula (b3), R¹² and R¹³ are each independently a hydrogen atom or a C₁-C₂₀ hydrocarbyl group which may contain a heteroatom. The hydrocarbyl group may be saturated or unsaturated, and may be linear, branched, or cyclic. Specific examples thereof include: a C₁-C₂₀ alkyl group such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, or an icosyl group; a C₃-C₂₀ cyclic saturated hydrocarbyl group such as a cyclopropyl group, a cyclopentyl group, a cyclohexyl group, a cyclopropylmethyl group, a 4-methylcyclohexyl group, a cyclohexylmethyl group, a norbornyl group, or an adamantyl group; a C₂-C₂₀ alkenyl group such as a vinyl group, a 1-propenyl group, a 2-propenyl group, a butenyl group, or a hexenyl group; a C₃-C₂₀ cyclic unsaturated hydrocarbyl group such as a cyclohexenyl group; a C₆-C₂₀ aryl group such as a phenyl group or a naphthyl group; a C₇-C₂₀ aralkyl group such as a benzyl group, a 1-phenylethyl group, or a 2-phenylethyl group; and a group obtained by combining these groups. Some or all of hydrogen atoms of the hydrocarbyl group may be replaced with a group containing a heteroatom such as an oxygen atom, a sulfur atom, a nitrogen atom, or a halogen atom, some of (—CH₂—)s in the hydrocarbyl group may be replaced with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like.

R¹² and R¹³ may be bonded to each other to form a ring together with a carbon atom to which they are bonded. Specific examples of the ring formed at this time include a cyclopropane ring, a cyclobutane ring, a cyclopentane ring, a cyclohexane ring, a norbornane ring, and an adamantane ring. Some or all of hydrogen atoms of the ring may be replaced with a group containing a heteroatom such as an oxygen atom, a sulfur atom, a nitrogen atom, or a halogen atom, some of (—CH₂—)s in the ring may be replaced with a group containing a heteroatom such as an oxygen atom, a sulfur atom, or a nitrogen atom, and as a result, the ring may contain a hydroxy group, a fluorine atom, a chlorine atom, a bromine atom, an iodine atom, a cyano group, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like.

In the formula (b3), R¹⁴ is a halogen atom, a hydroxy group, a cyano group, a nitro group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A}) (R^{14B}). R^{14A} and R^{14B} are each independently a hydrogen atom or a C₁-C₆ hydrocarbyl group. The halogen atom is preferably a fluorine atom, a chlorine atom, a bromine atom, or an iodine atom, and more preferably a fluorine atom or an iodine atom. The hydrocarbyl group and hydrocarbyl moieties of the hydrocarbyloxy group, the hydrocarbyloxycarbonyl group, and the hydrocarbylthio group may be saturated or unsaturated, and may be linear, branched, or cyclic. Specific examples thereof include those similar to those exemplified as the hydrocarbyl group of R¹² and R¹³. Some or all of hydrogen atoms of the hydrocarbyl group may be replaced with a group containing a heteroatom such as an oxygen atom, a sulfur atom, a nitrogen atom, or a halogen atom, some of (—CH₂—)s in the hydrocarbyl group may be replaced with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic acid anhydride (—C(═O)—O—C(—O)—), a haloalkyl group, or the like. When b2 is 2 or more, R¹⁴s may be the same as or different from each other.

When b2 is 2 or more, a plurality of R¹⁴s may be bonded to each other to form a ring together with aromatic ring carbon atoms to which they are bonded. Specific examples of the ring formed at this time include a cyclopropane ring, a cyclobutane ring, a cyclopentane ring, a cyclohexane ring, a norbornane ring, and an adamantane ring. Some or all of hydrogen atoms of the ring may be replaced with a group containing a heteroatom such as an oxygen atom, a sulfur atom, a nitrogen atom, or a halogen atom, some of (—CH₂—)s in the ring may be replaced with a group containing a heteroatom such as an oxygen atom, a sulfur atom, or a nitrogen atom, and as a result, the ring may contain a hydroxy group, a fluorine atom, a chlorine atom, a bromine atom, an iodine atom, a cyano group, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like.

Specific examples of the repeat unit b3 include, but are not limited to, those listed below. Note that, in the following formulas, R^{A} is as defined above, and Me is a methyl group. Bonding positions of various substituents on an aromatic ring may be replaced with each other.

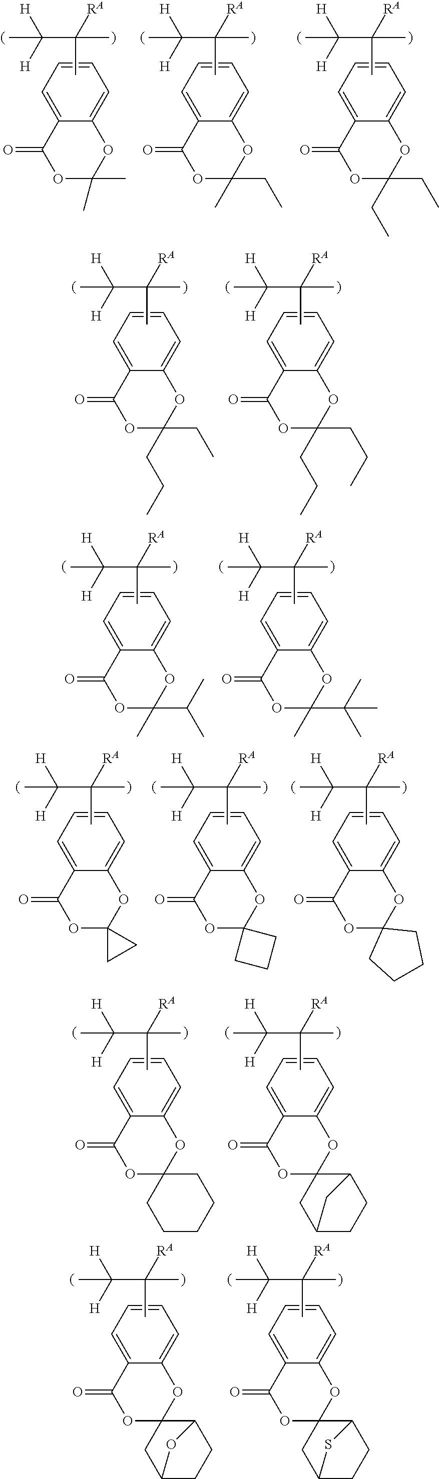

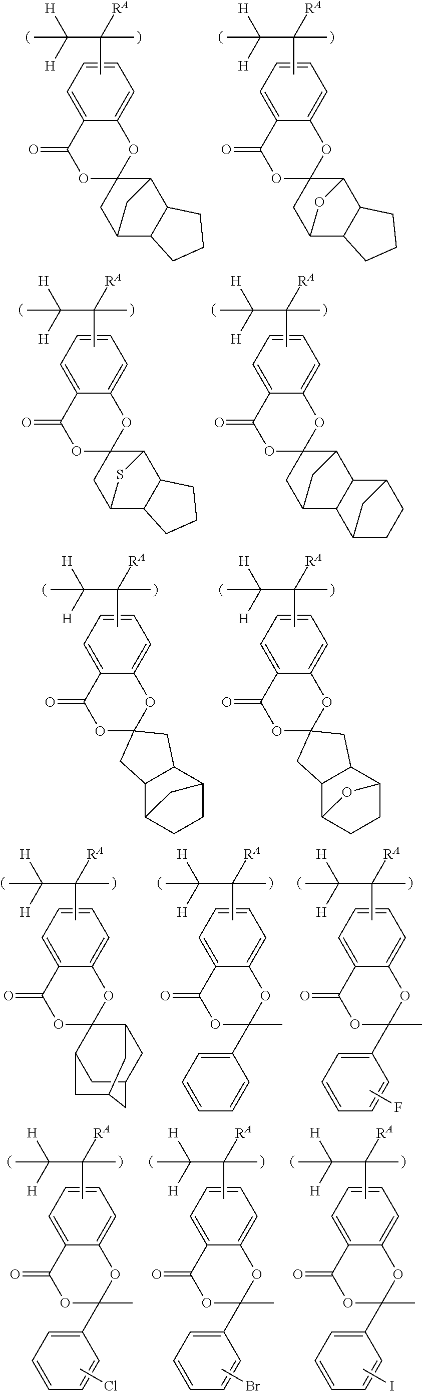

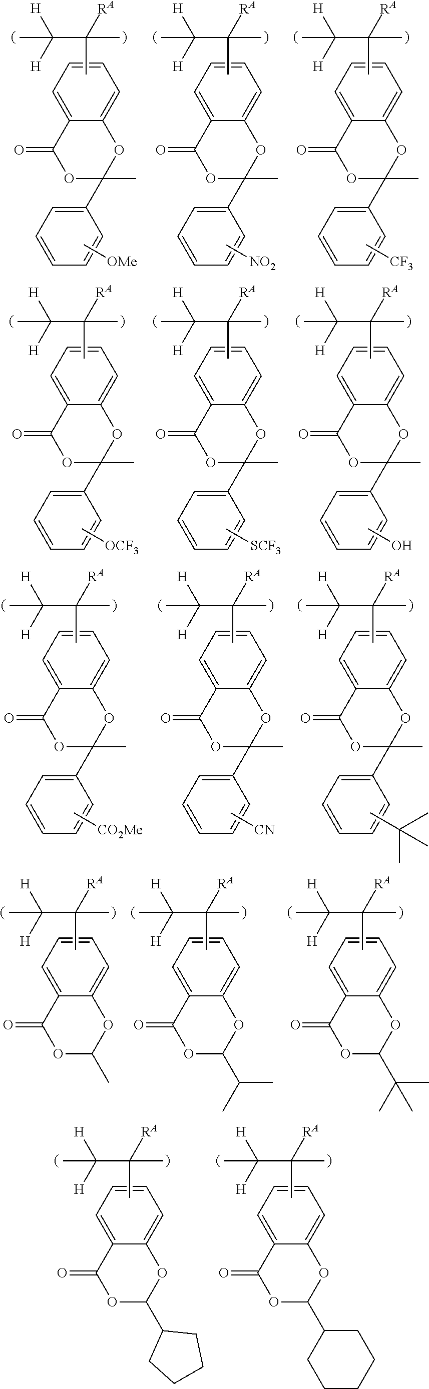

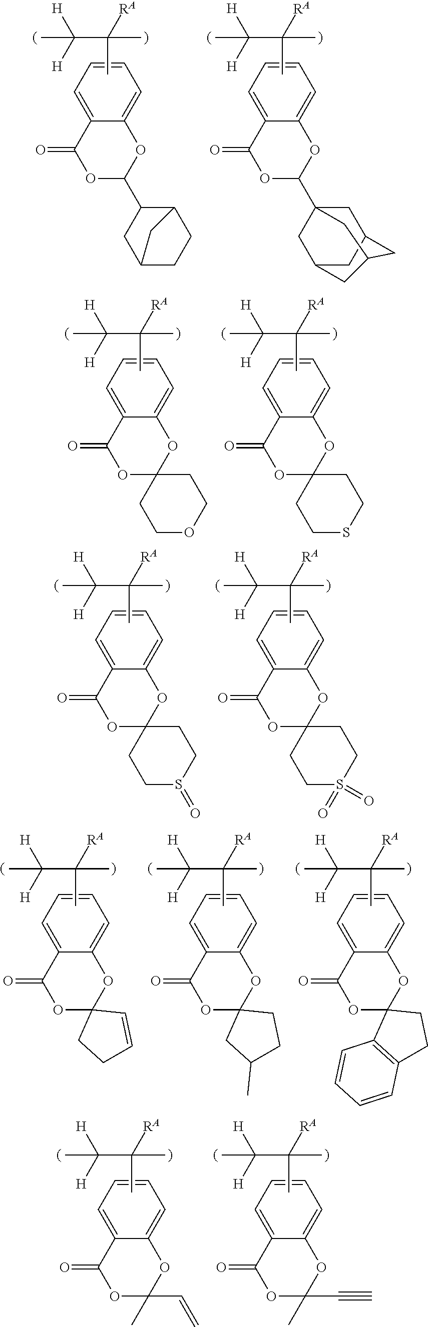

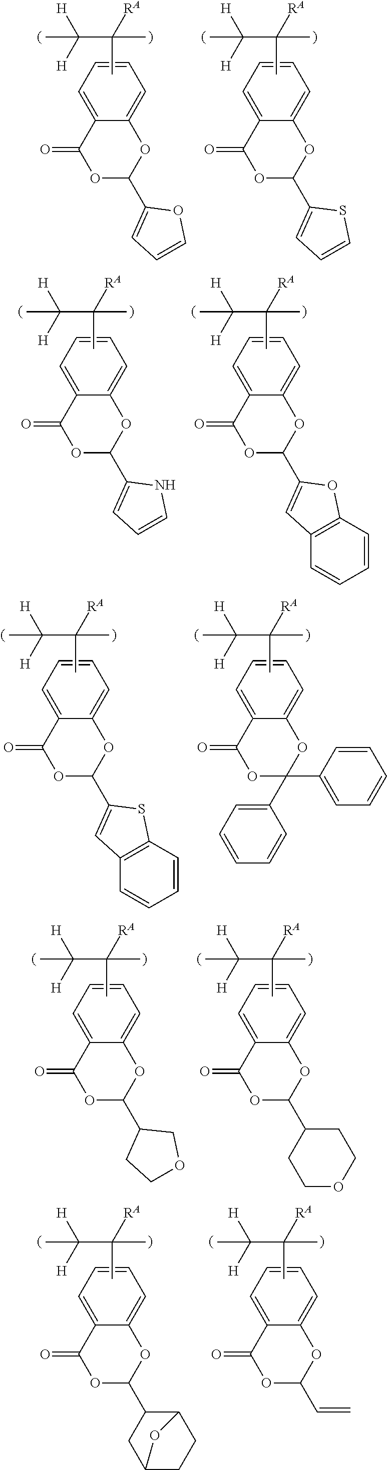

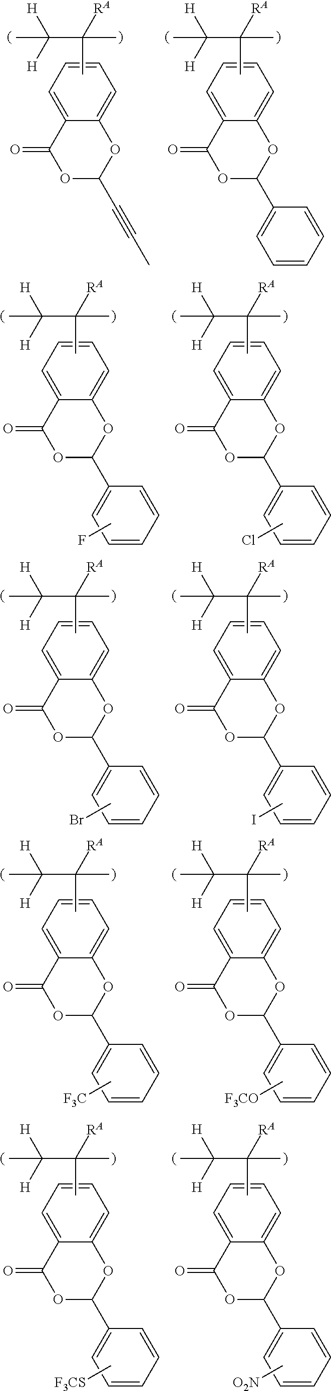

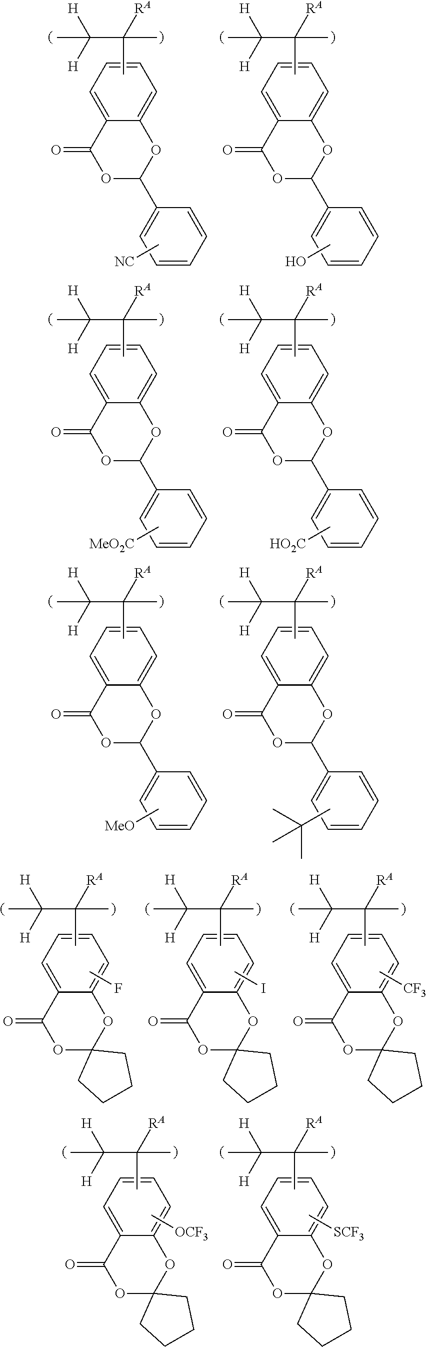

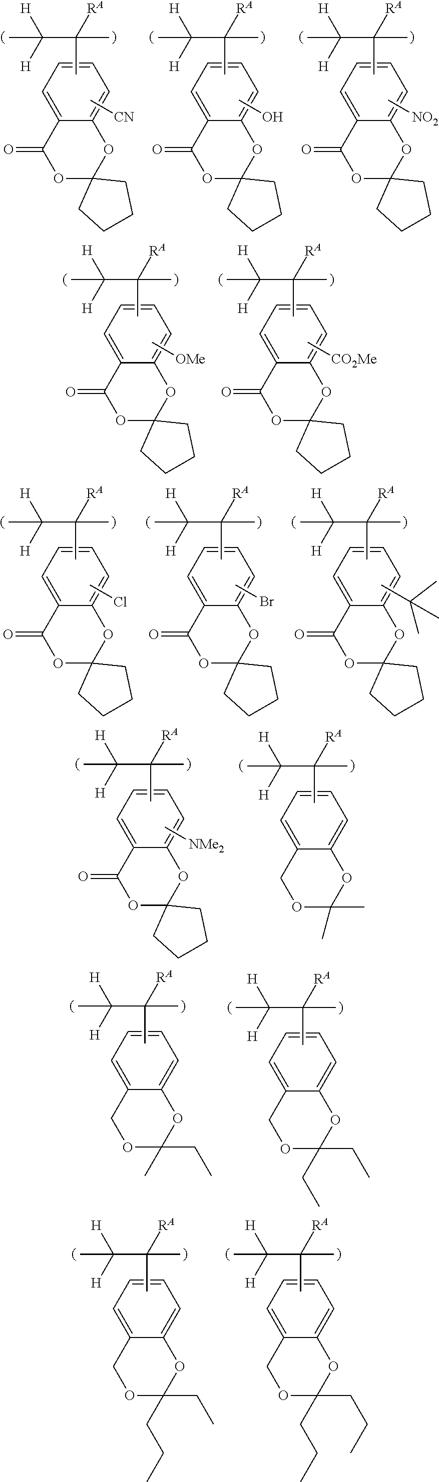

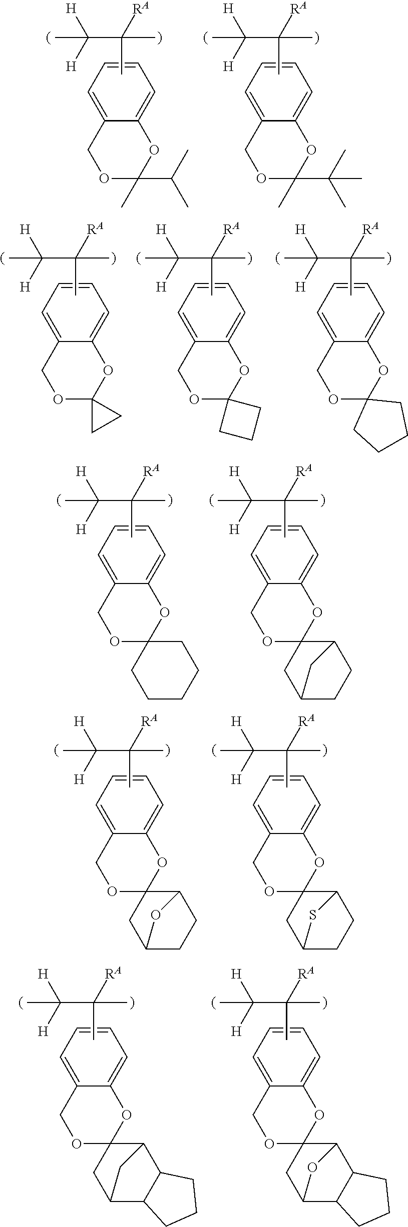

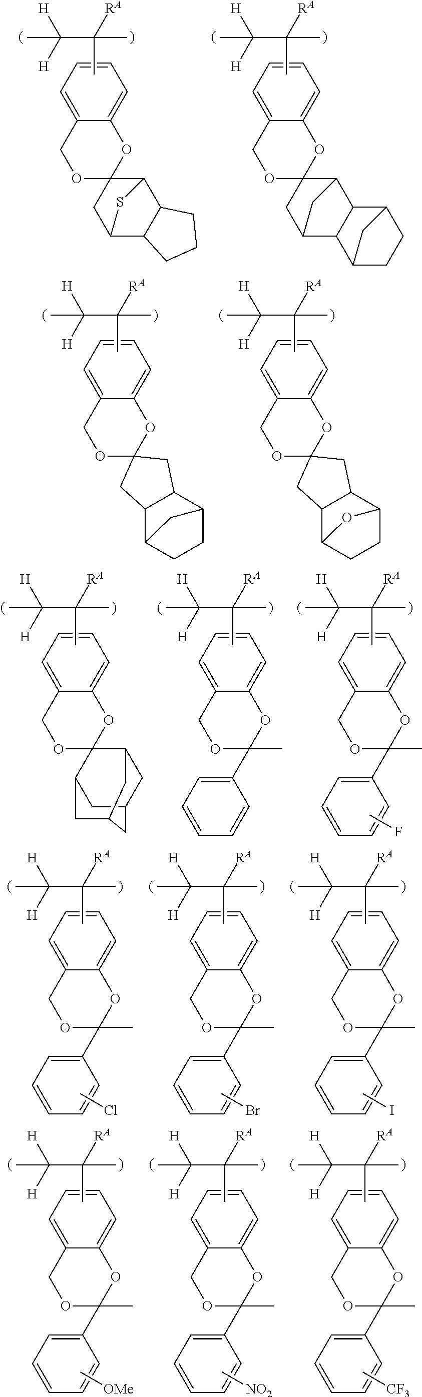

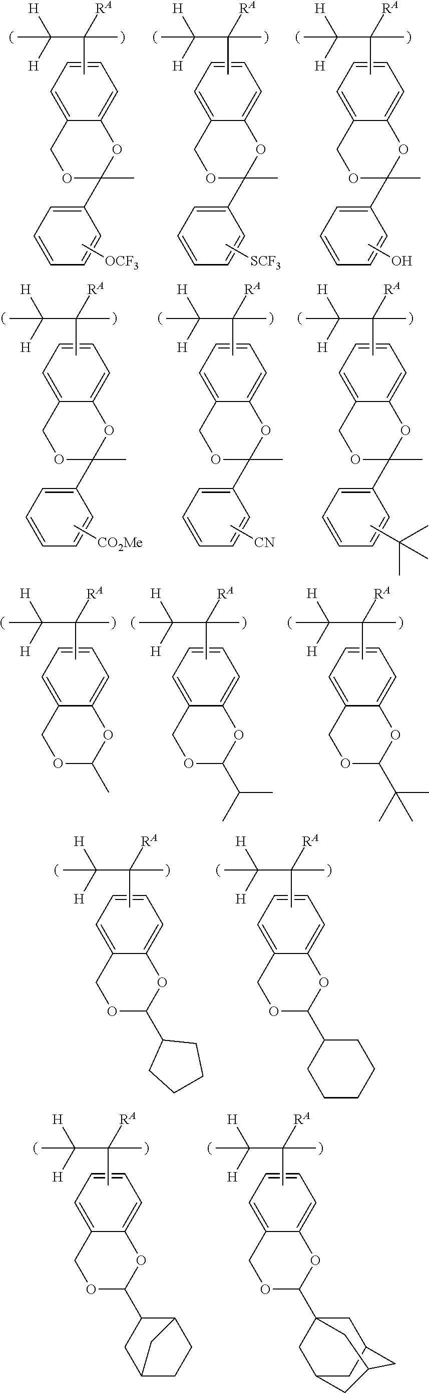

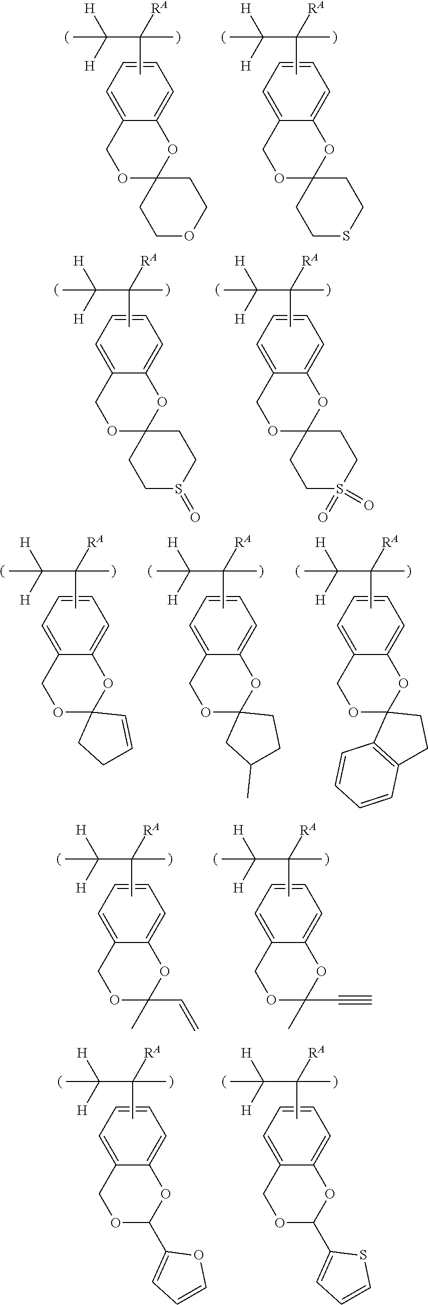

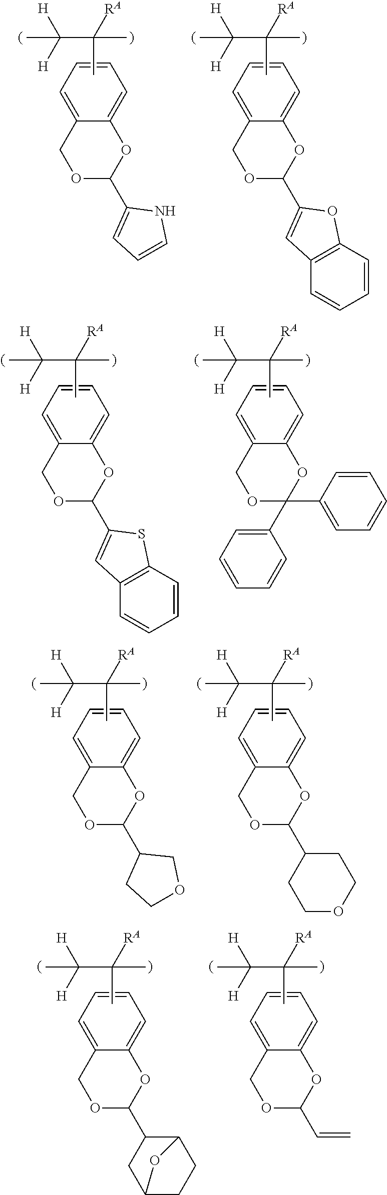

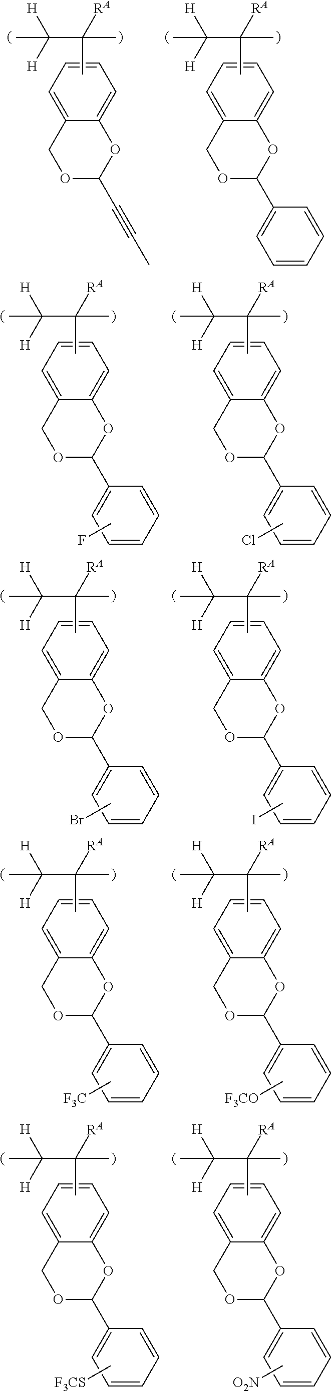

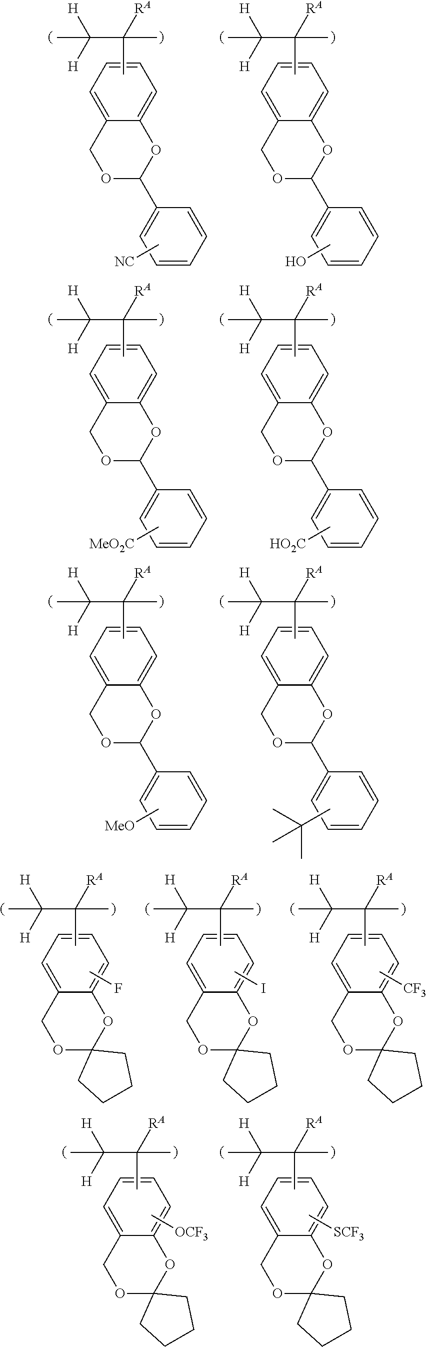

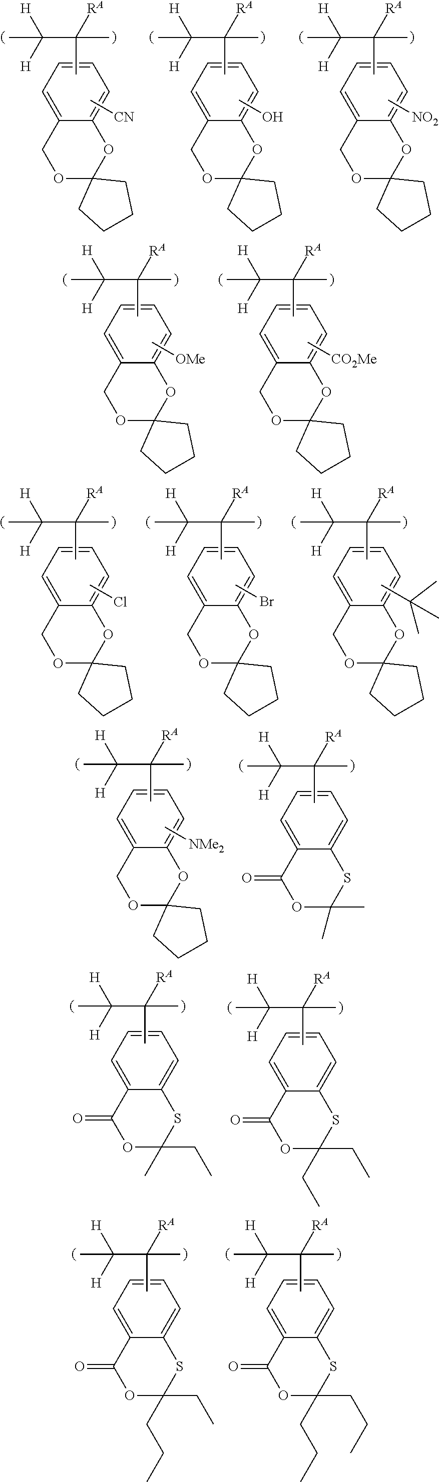

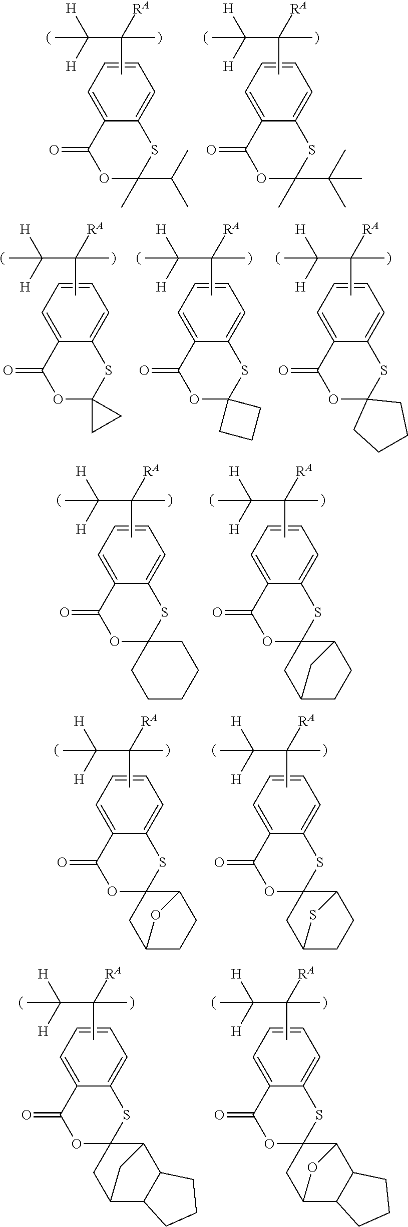

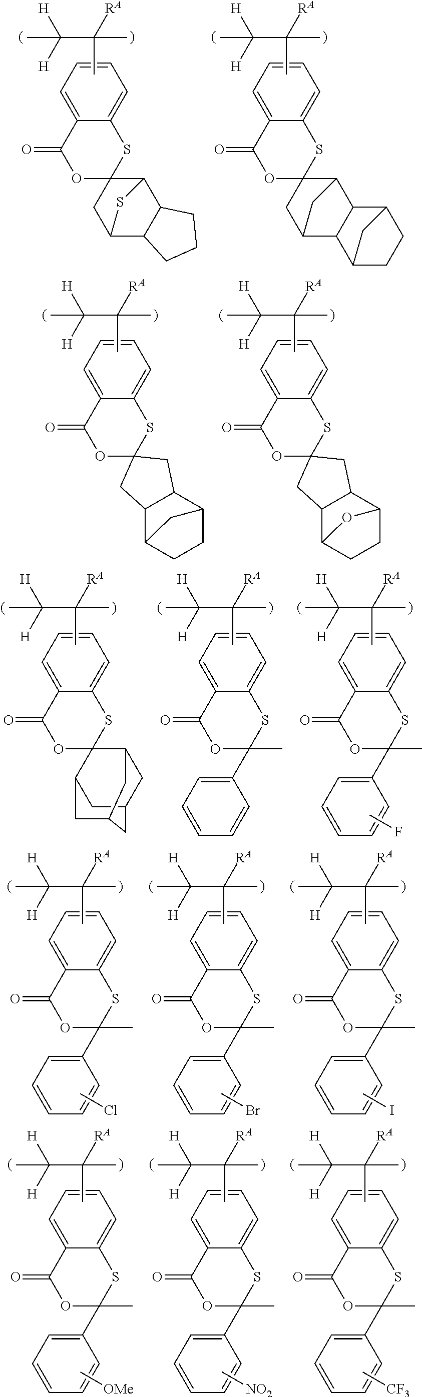

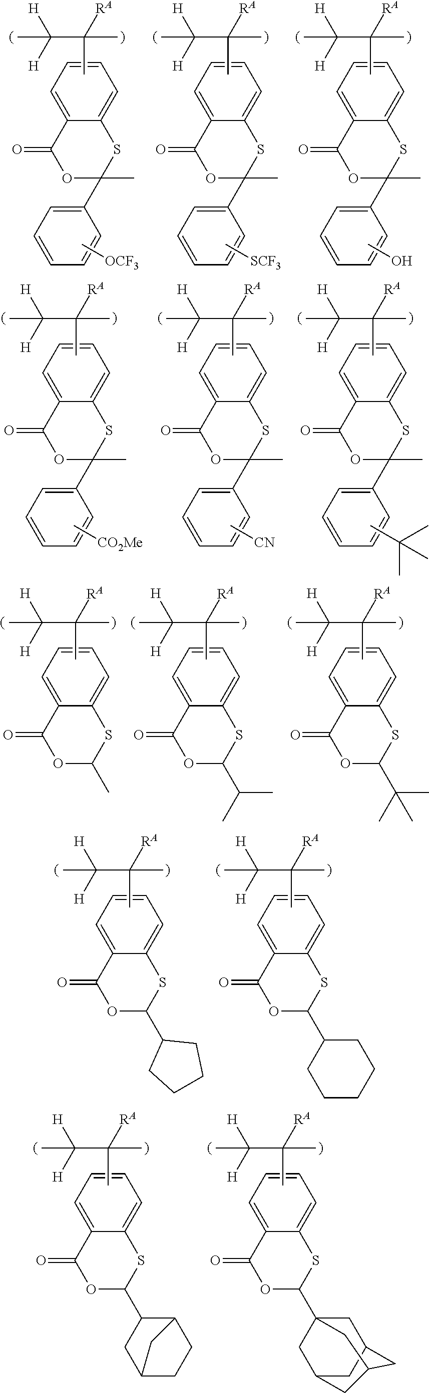

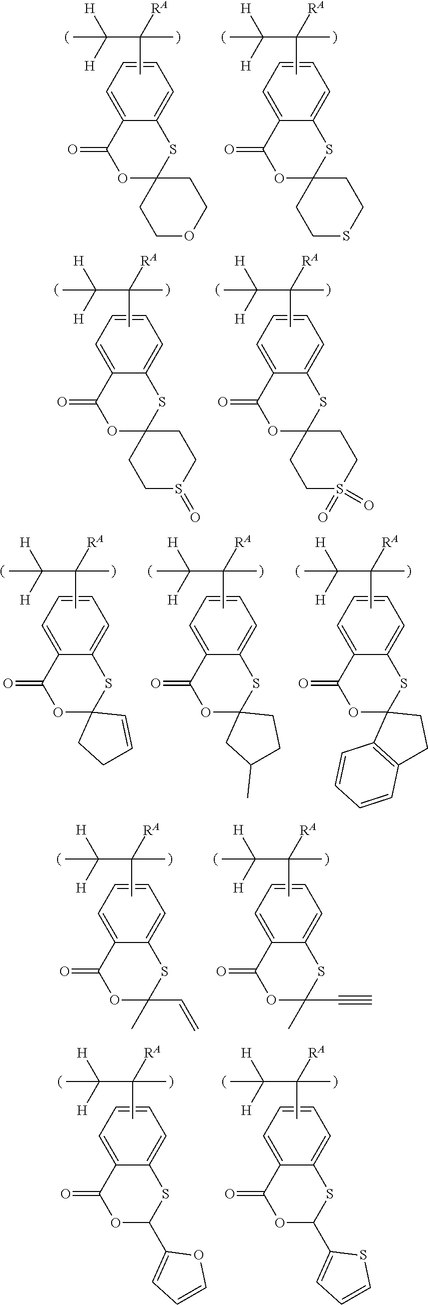

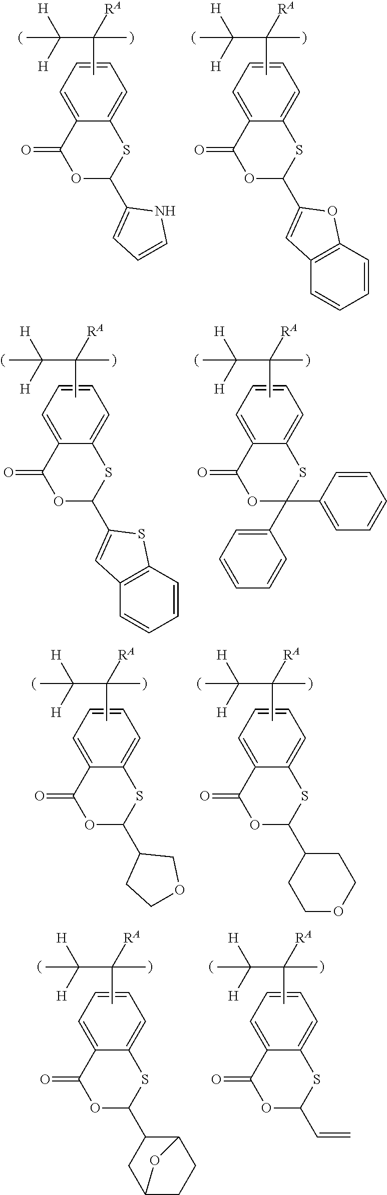

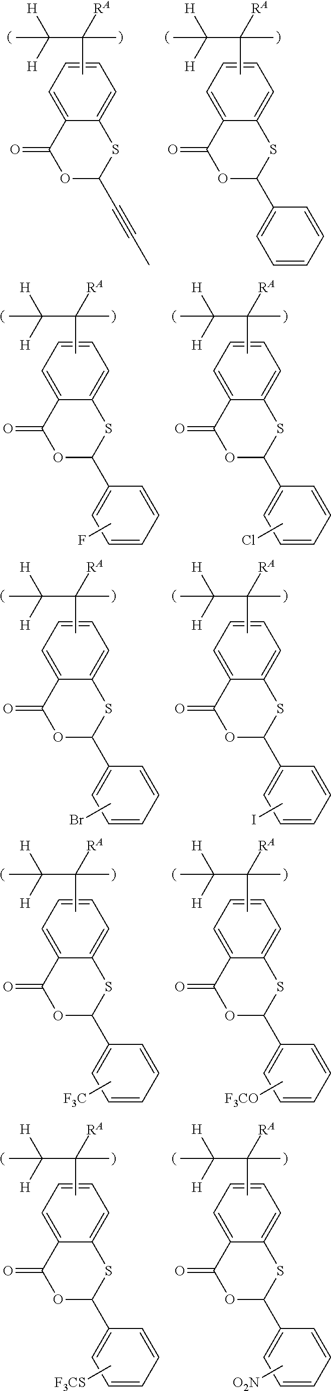

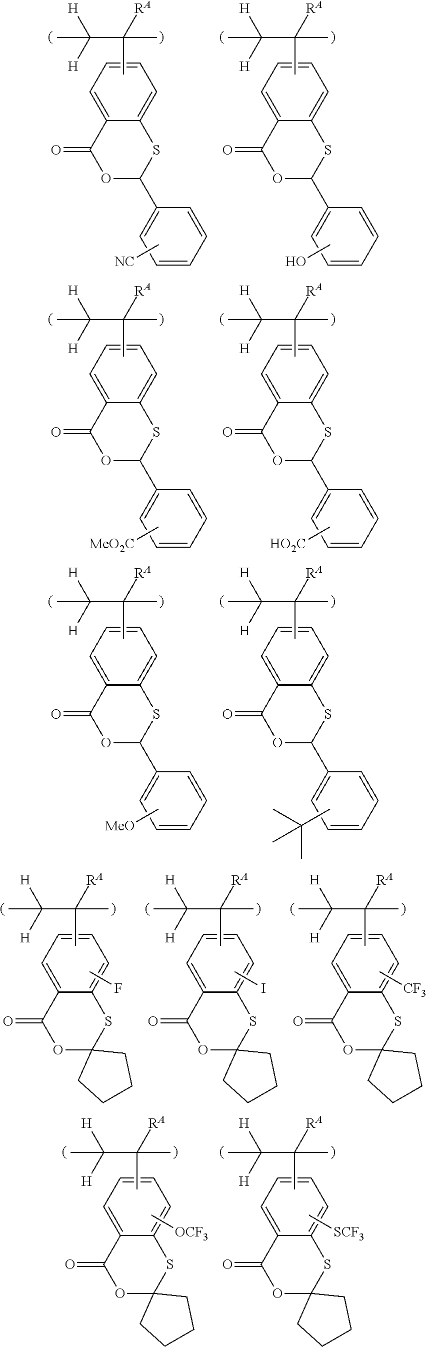

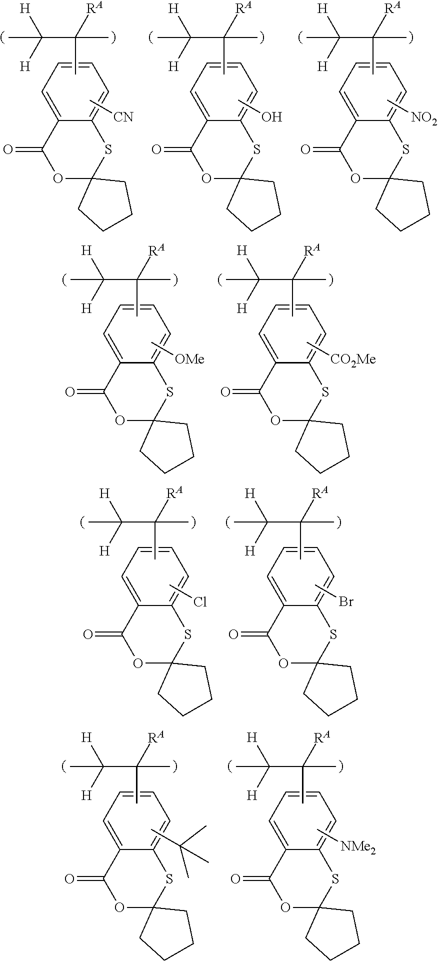

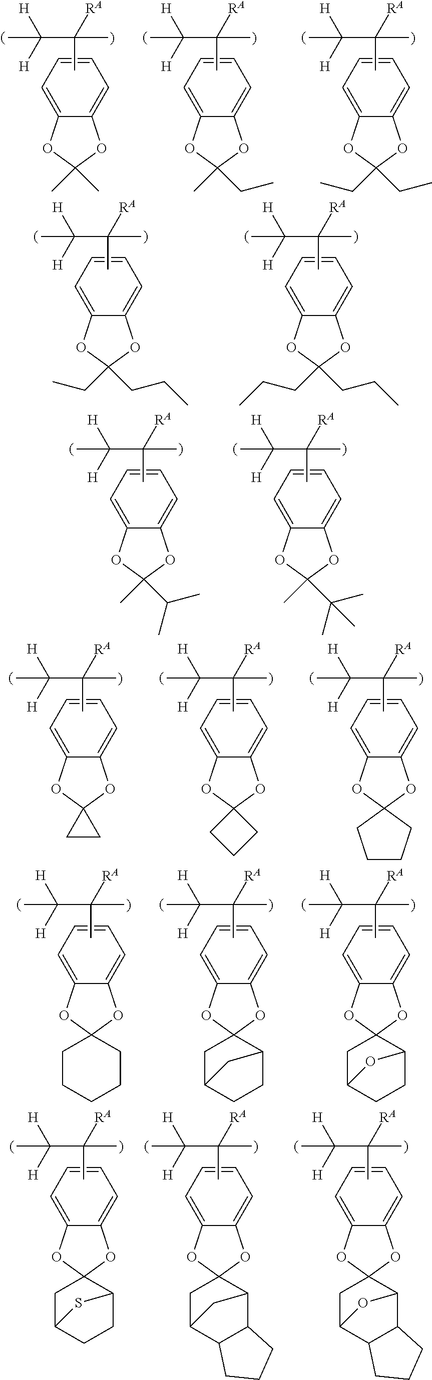

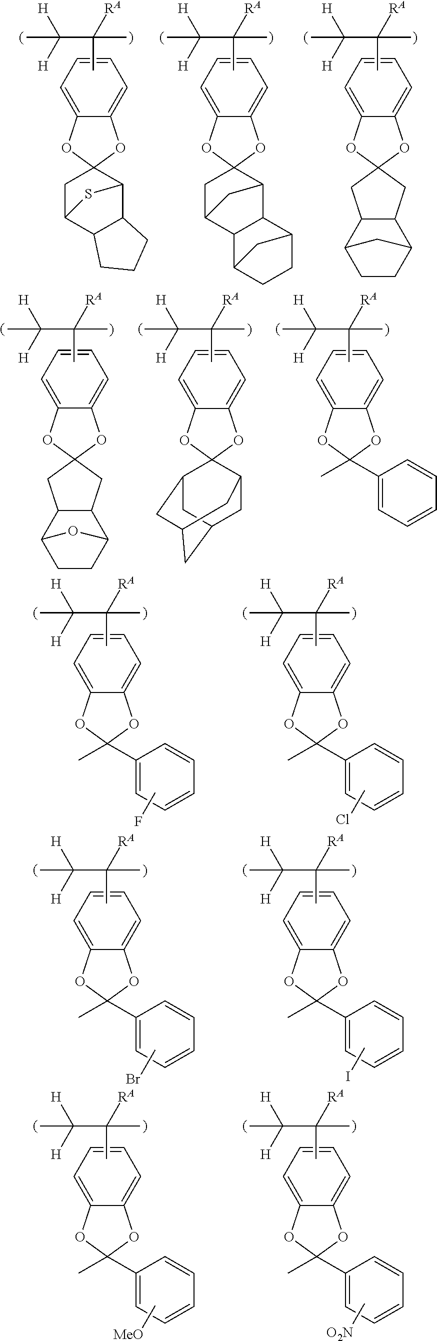

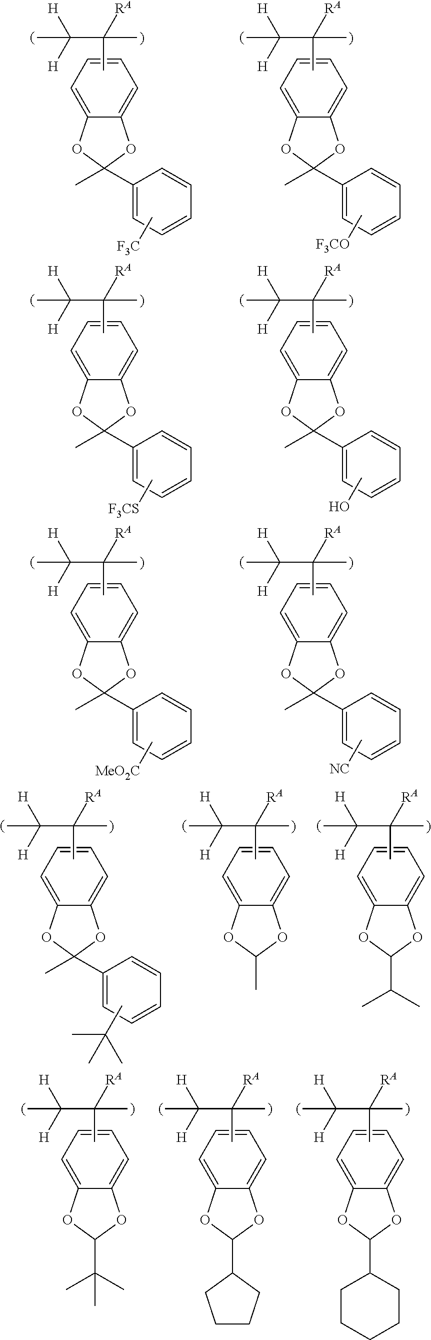

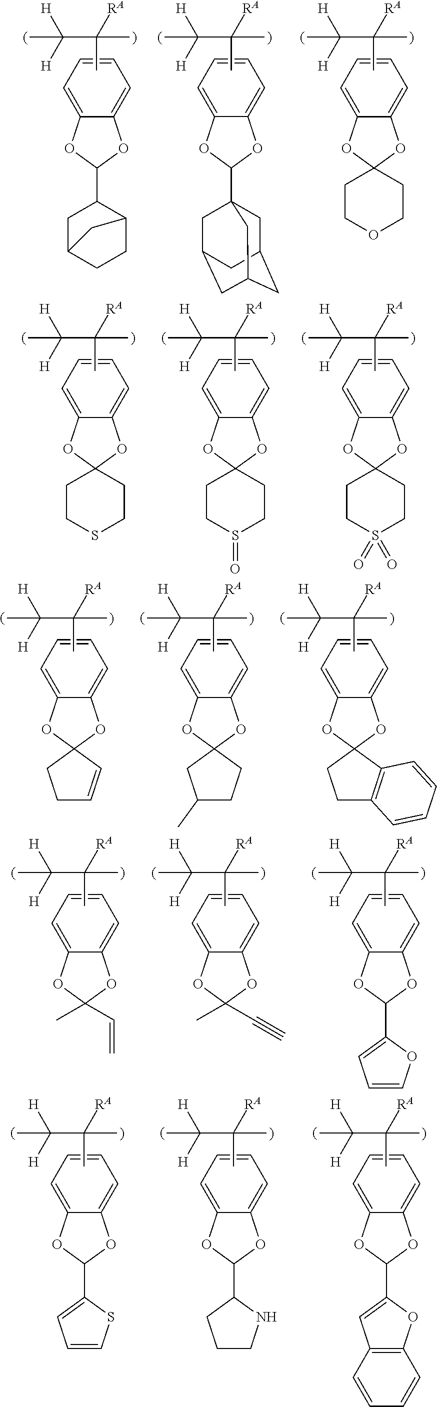

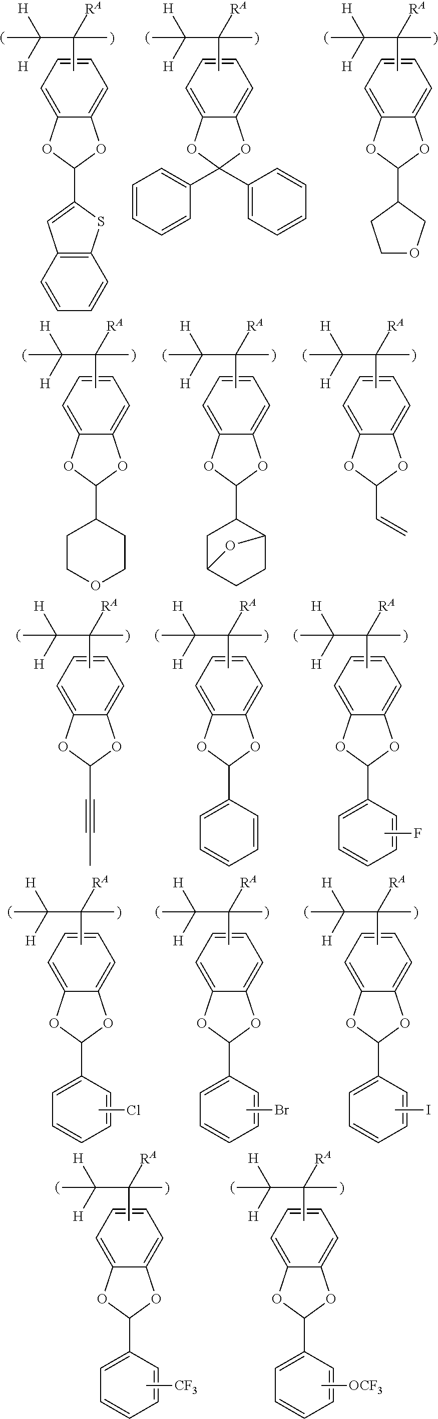

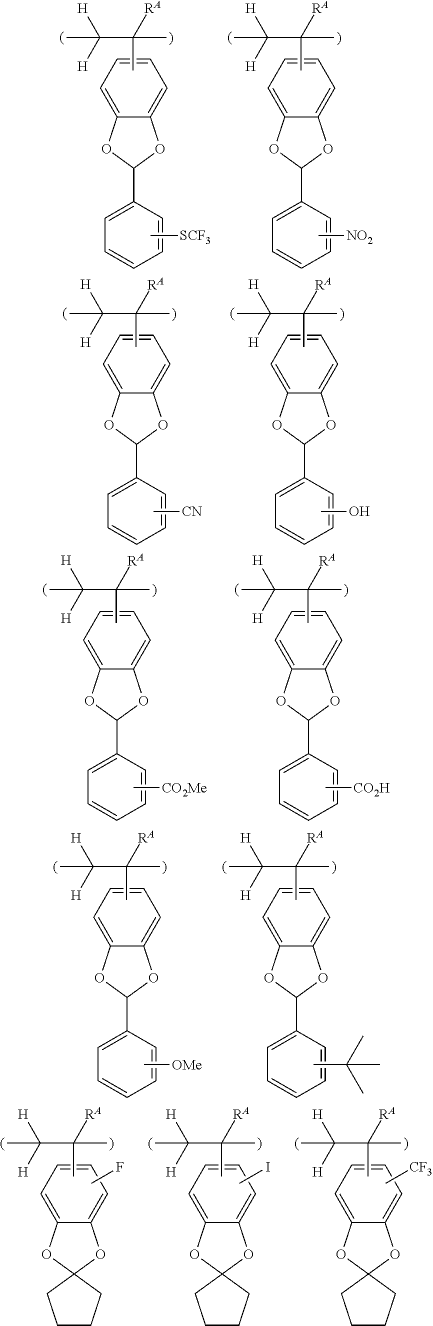

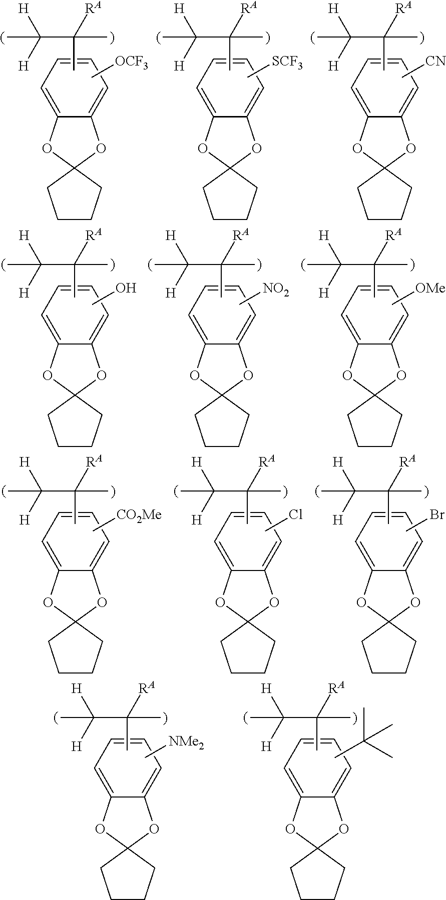

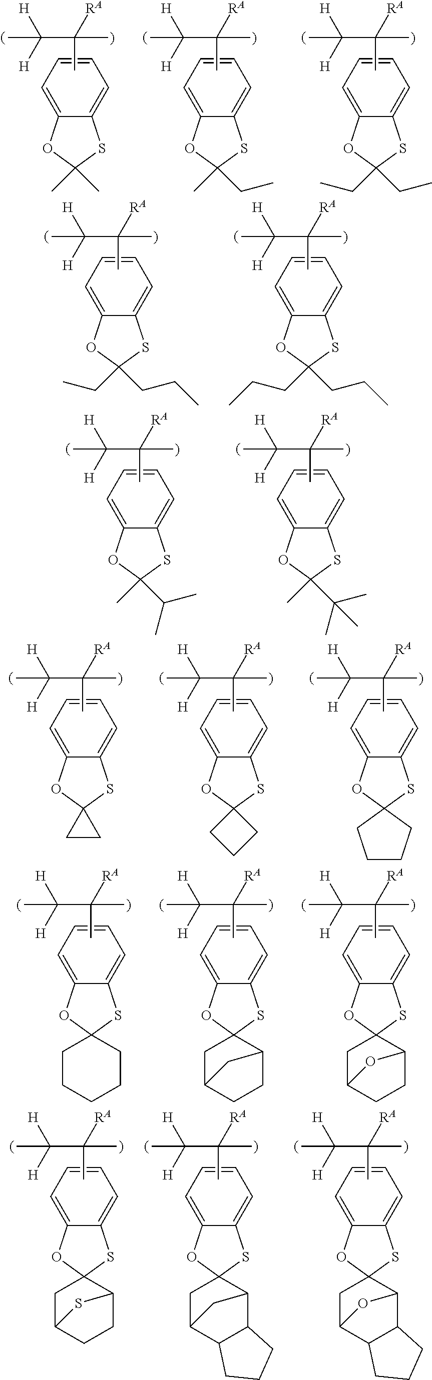

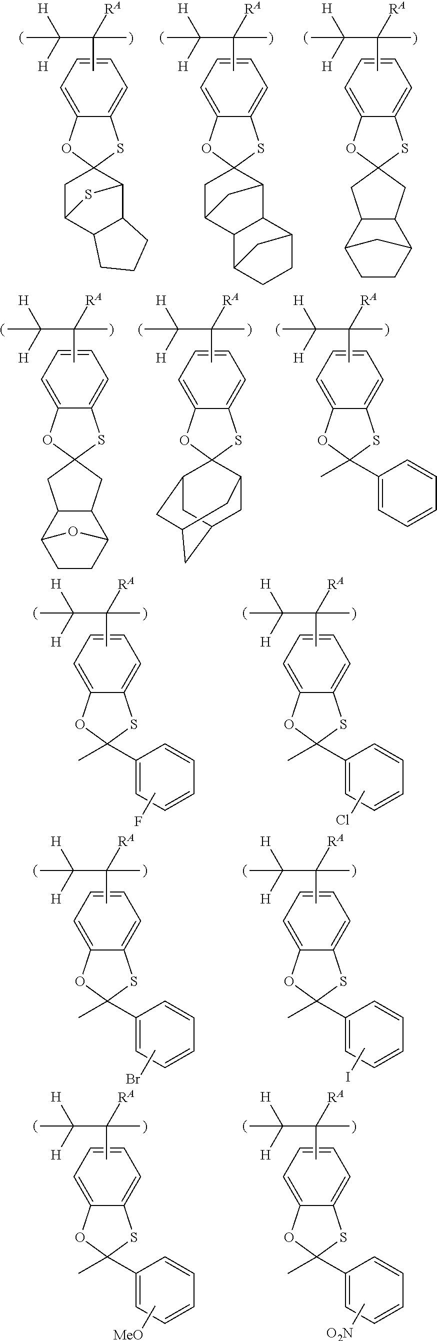

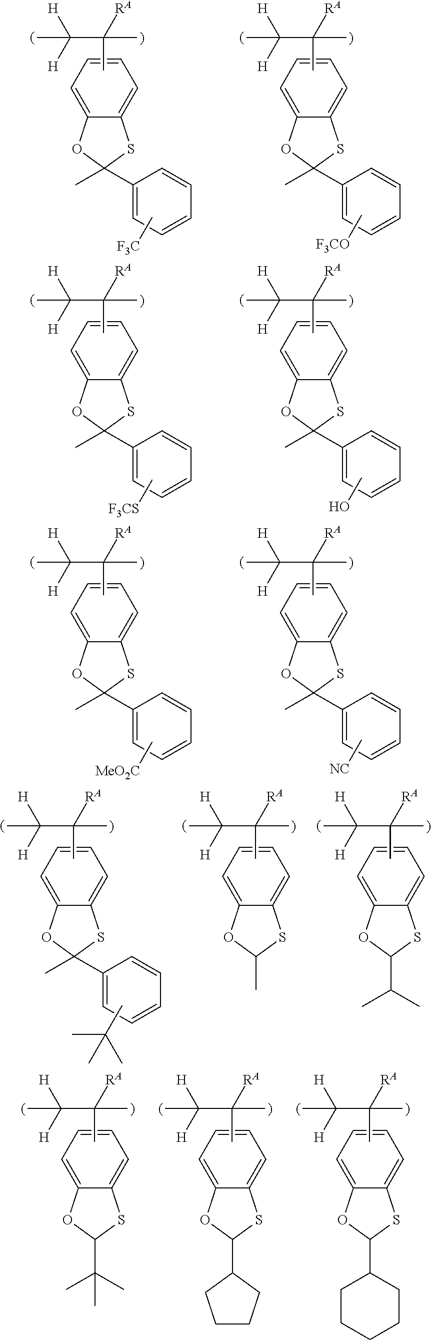

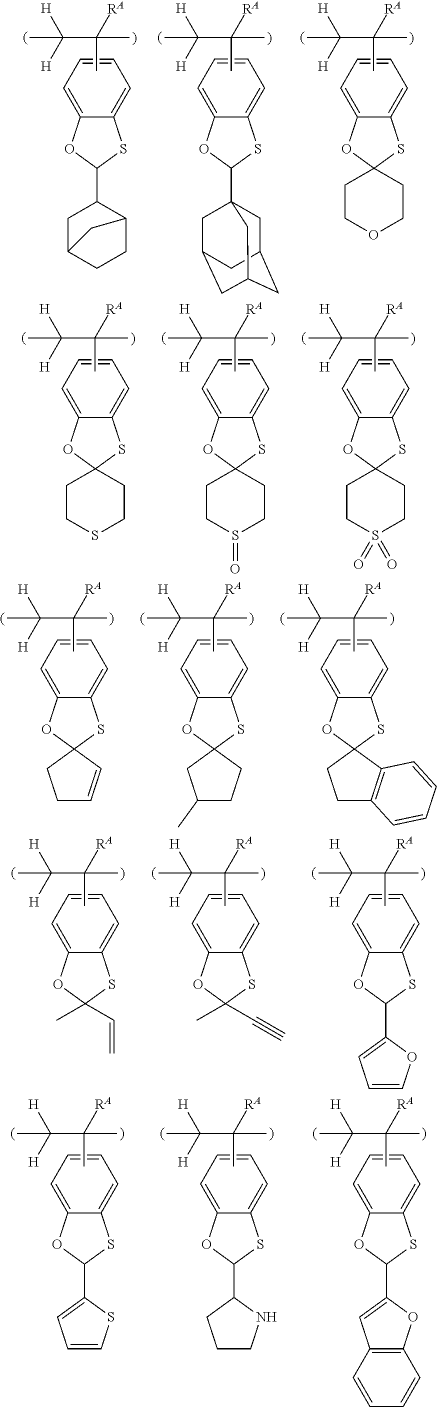

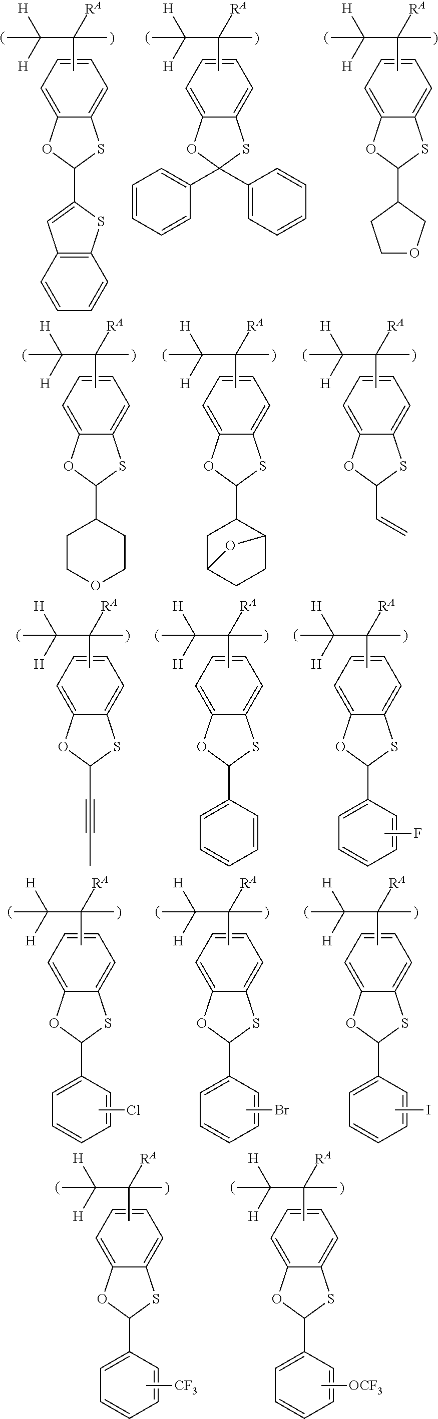

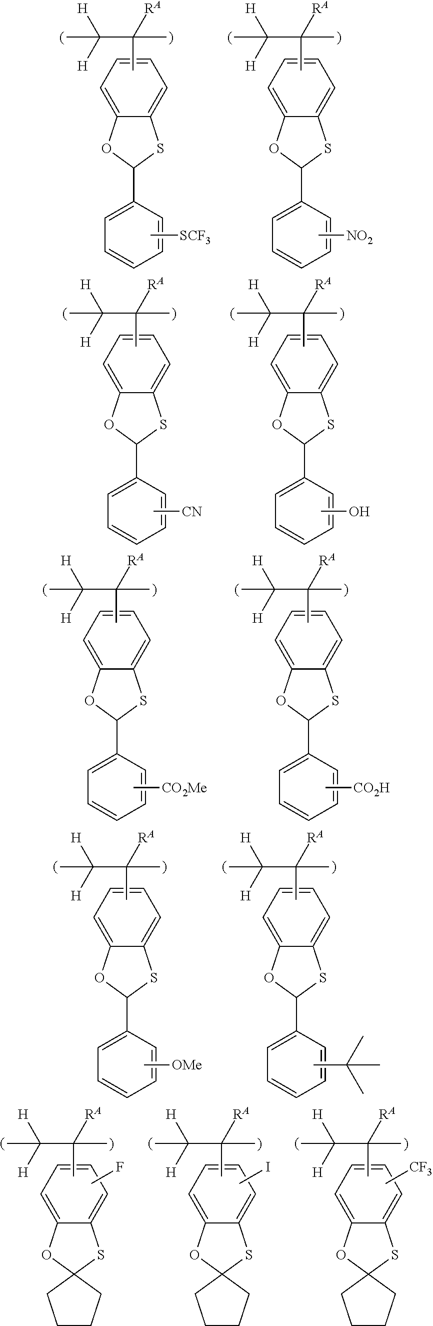

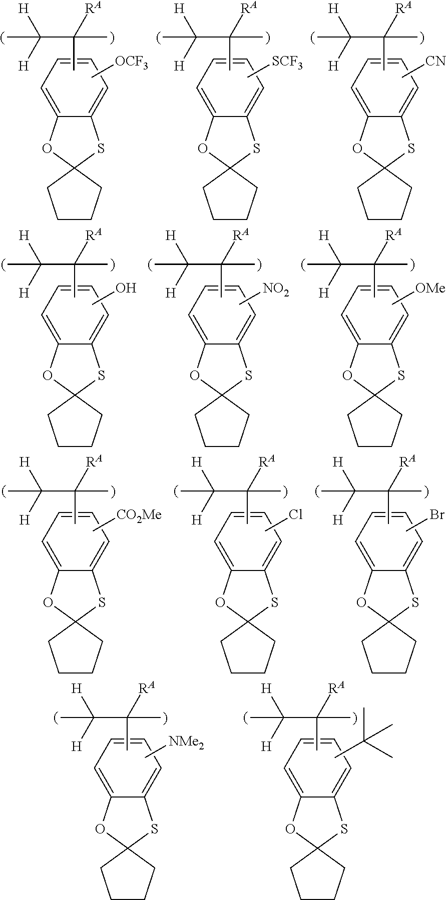

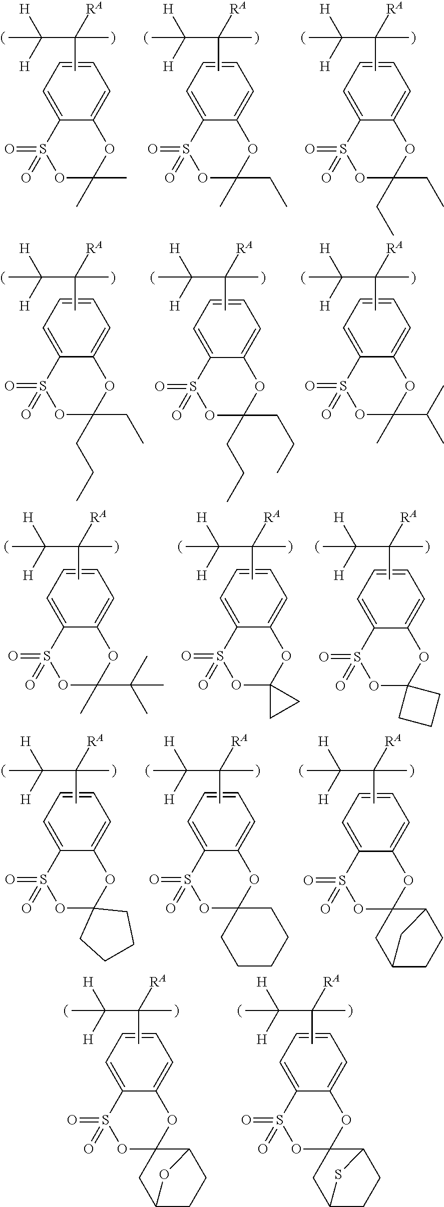

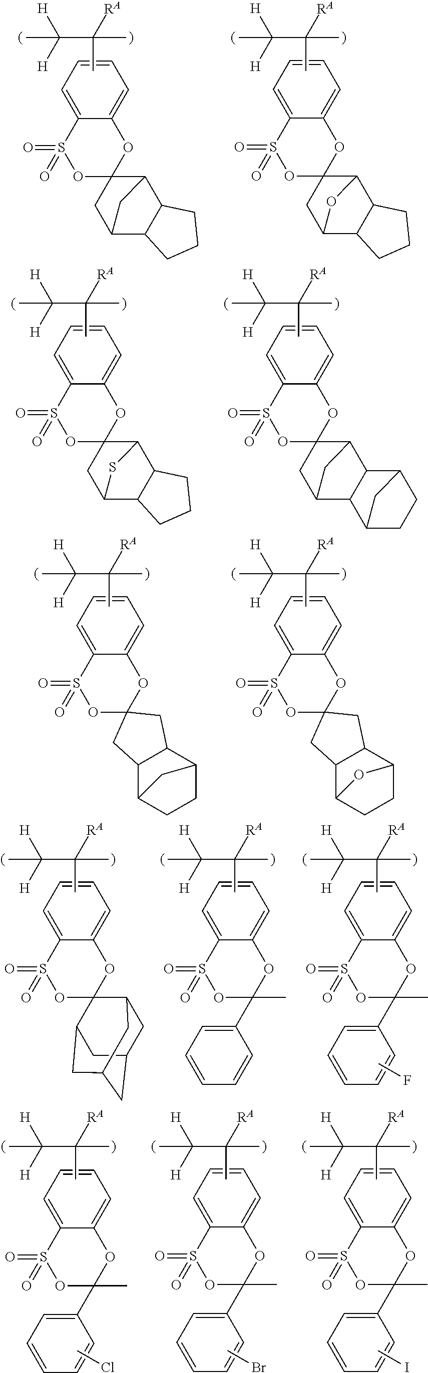

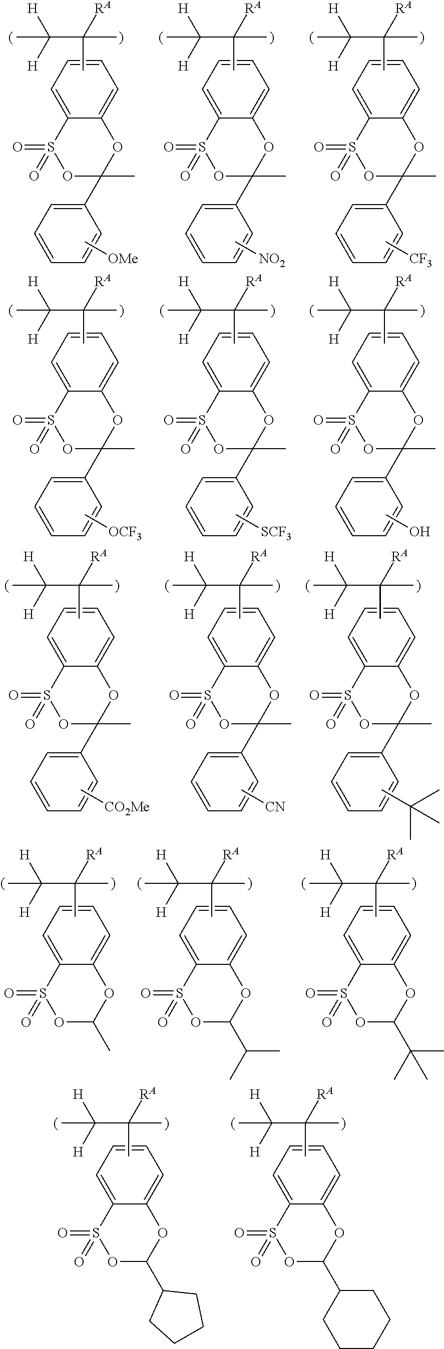

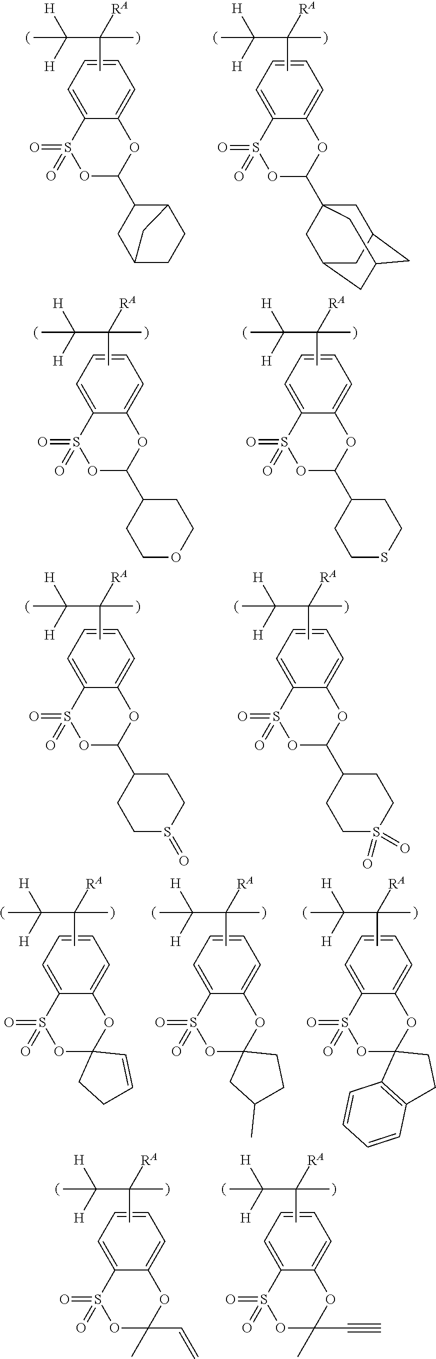

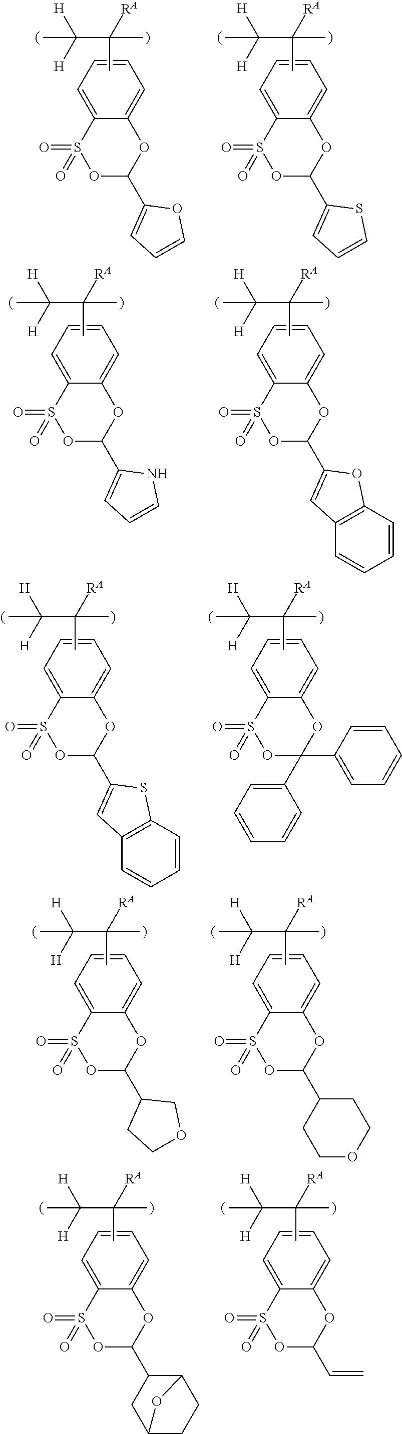

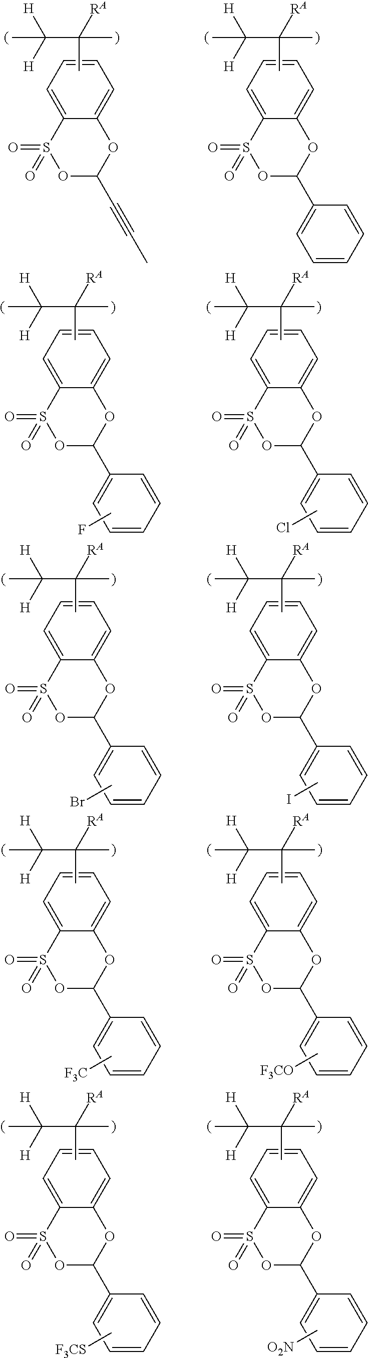

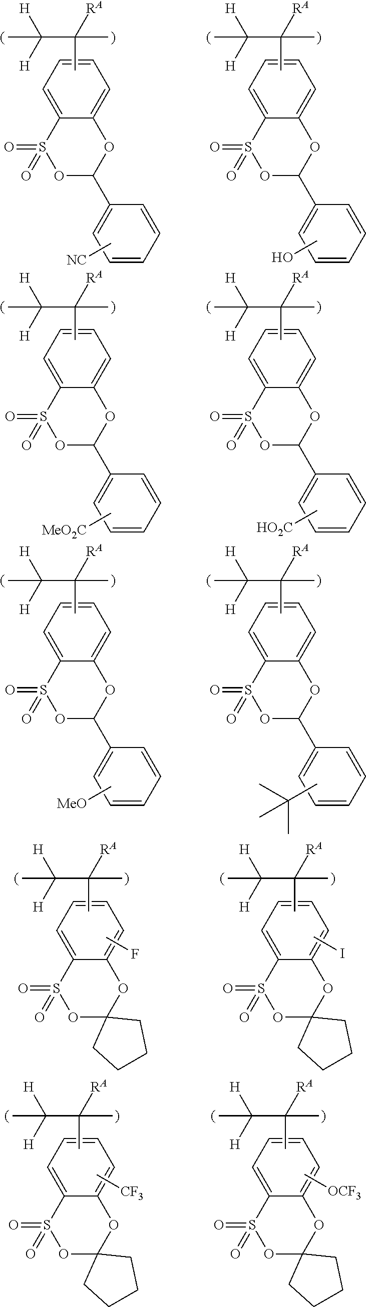

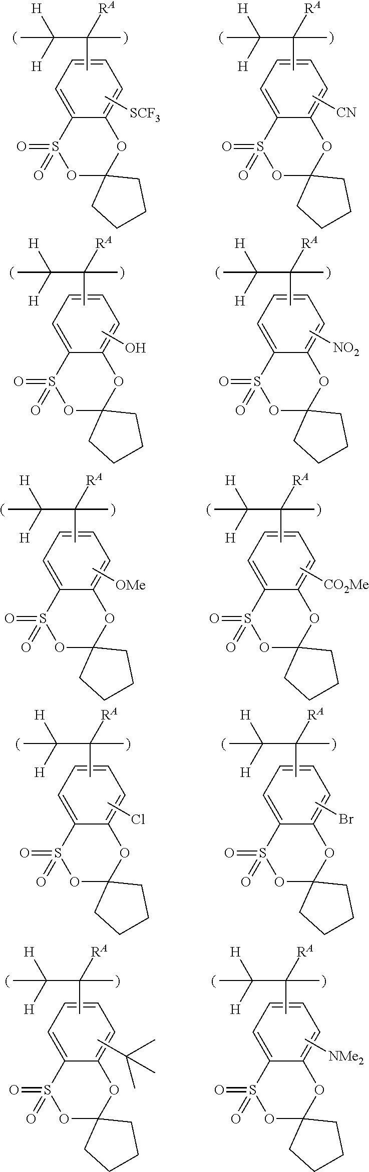

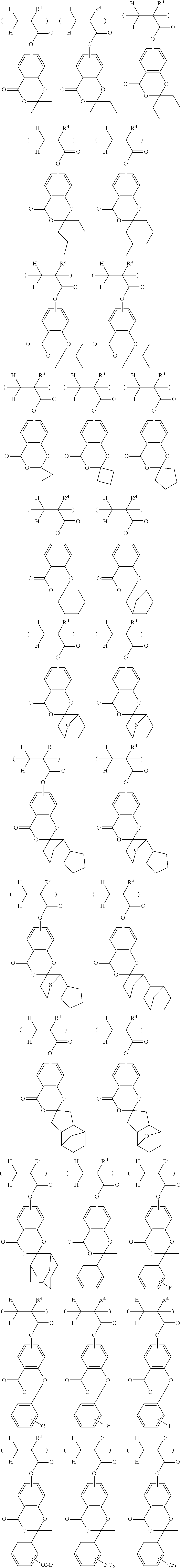

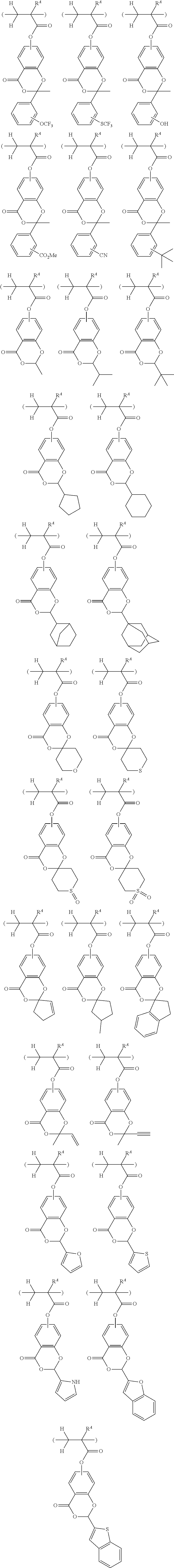

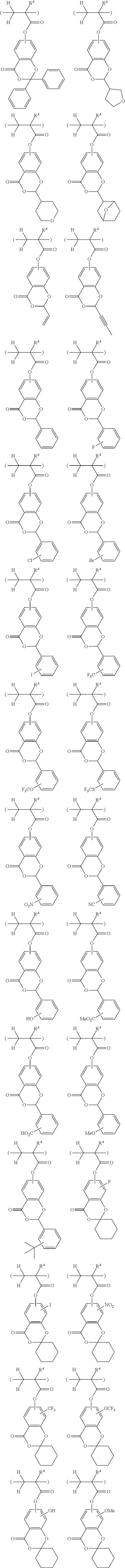

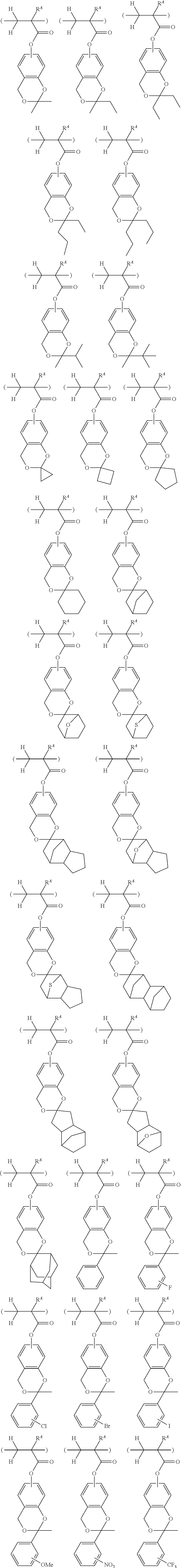

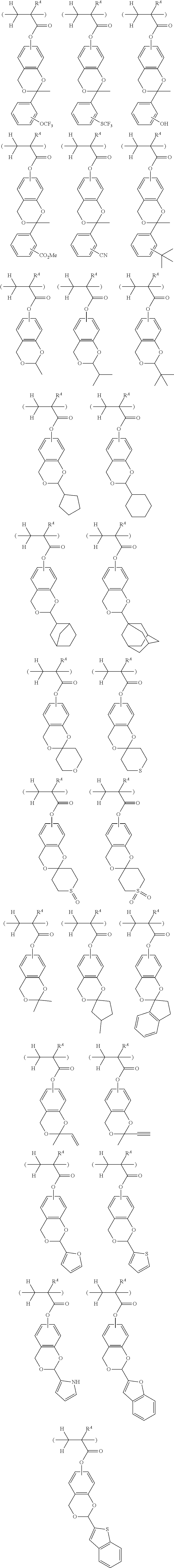

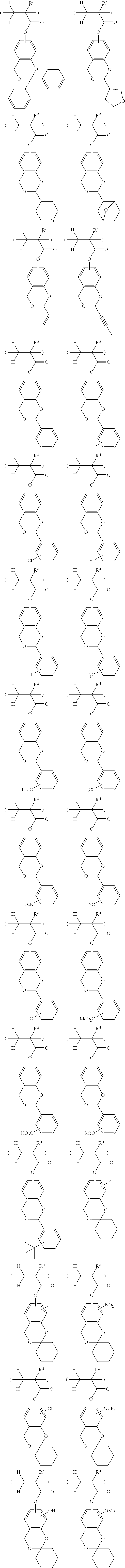

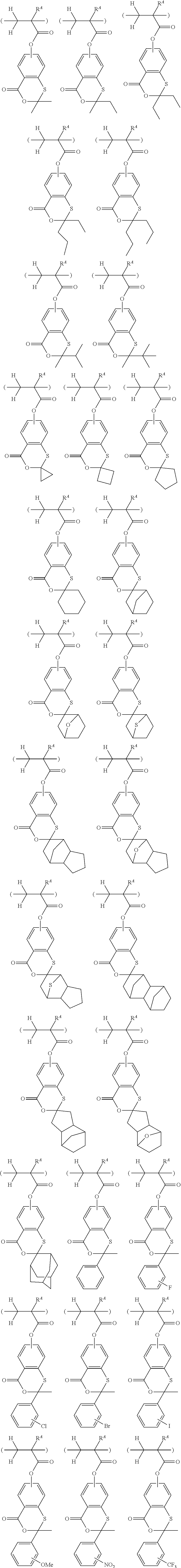

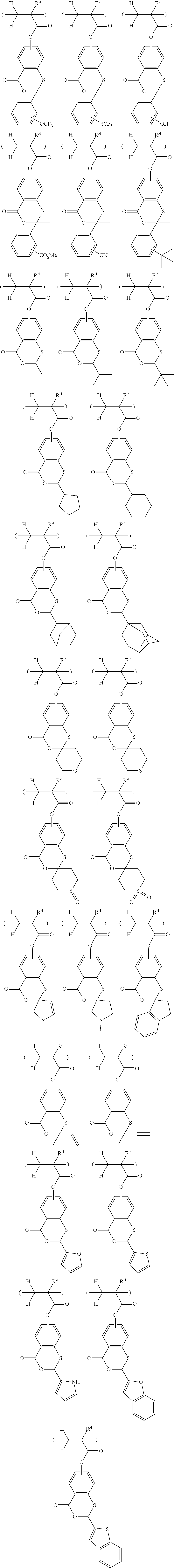

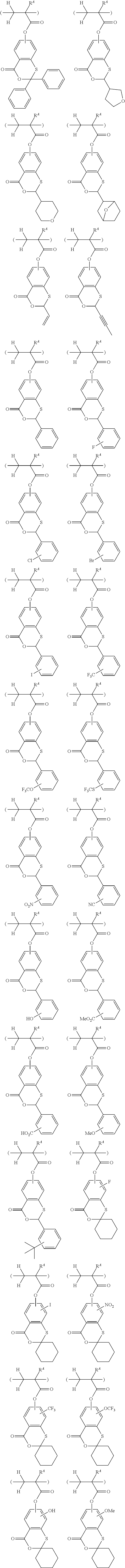

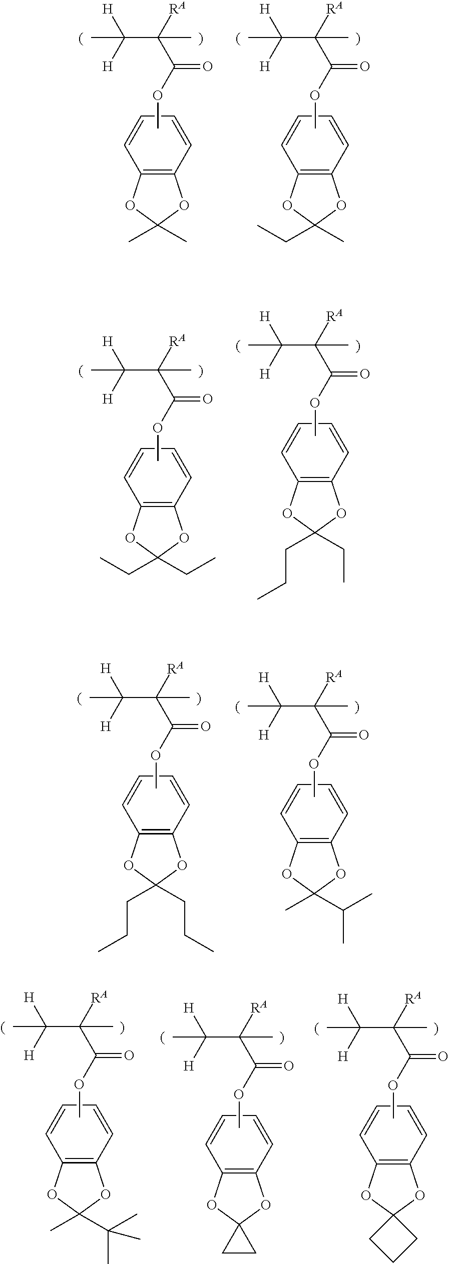

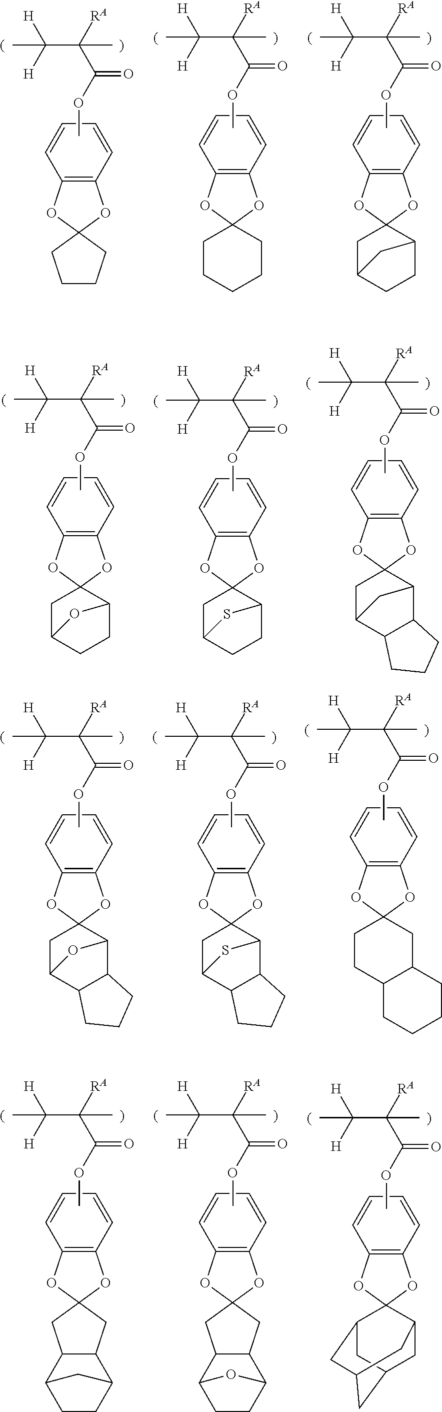

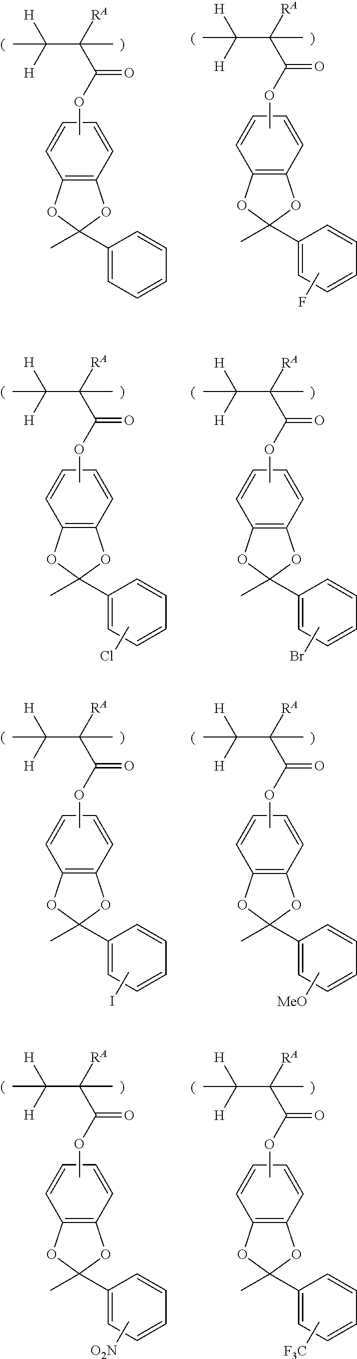

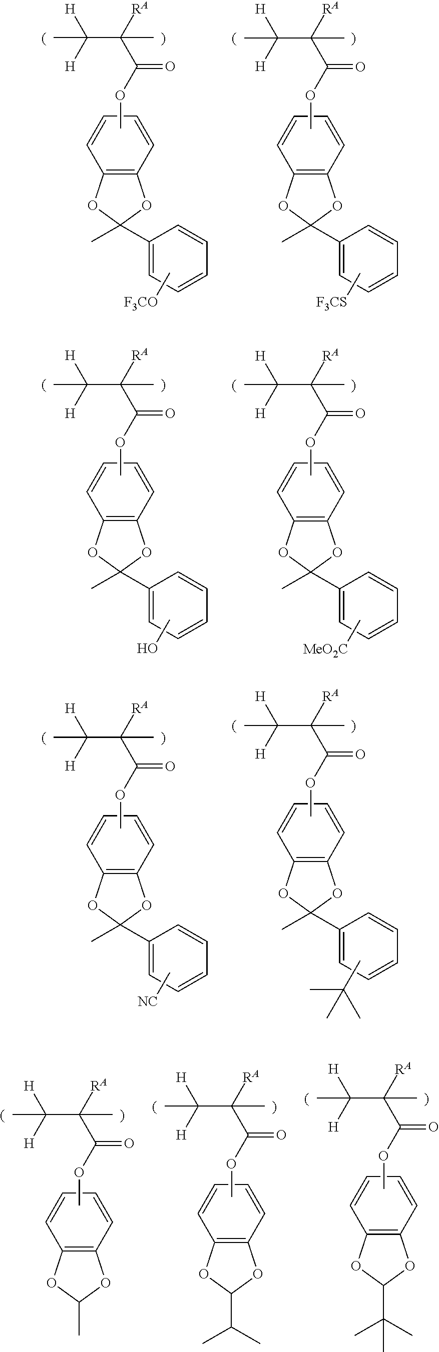

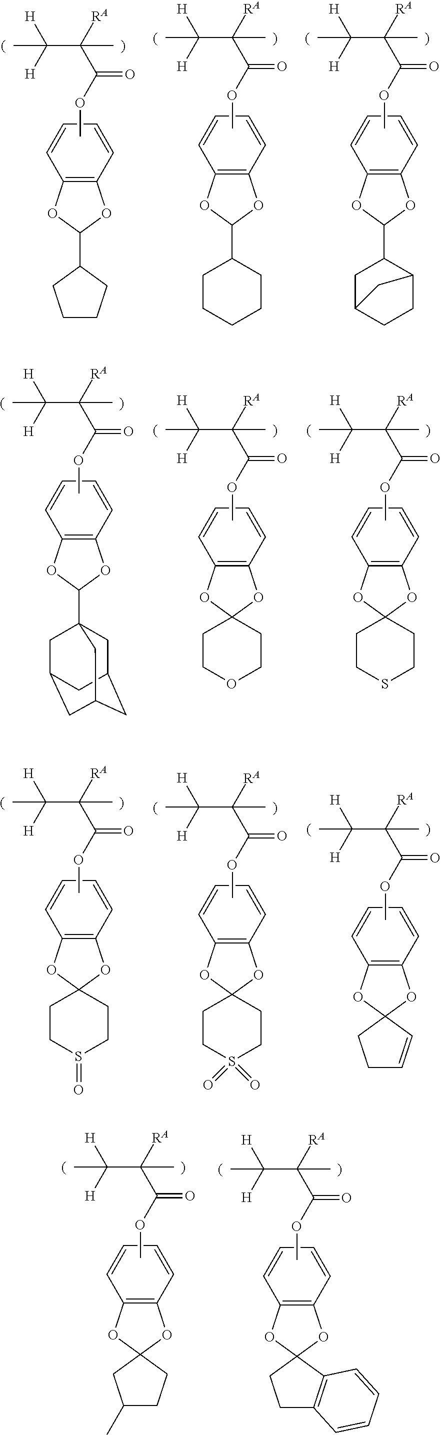

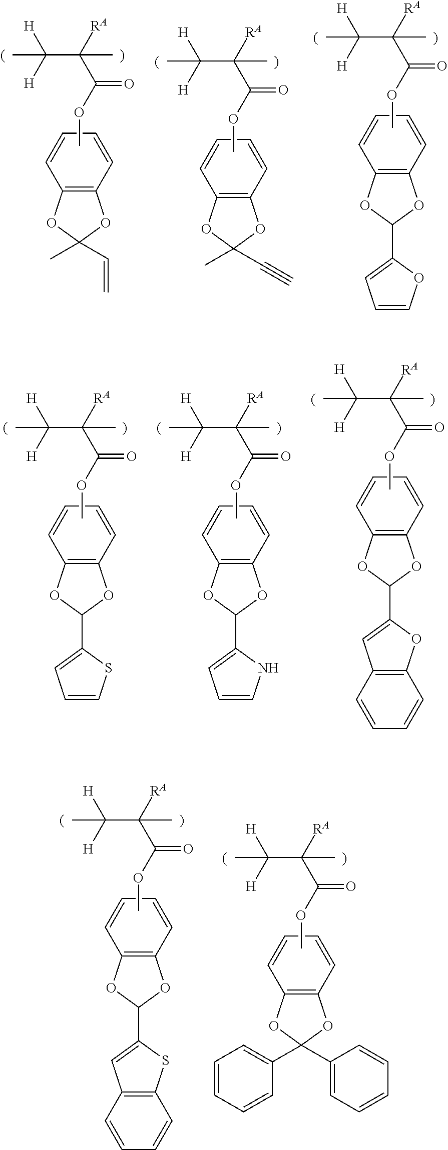

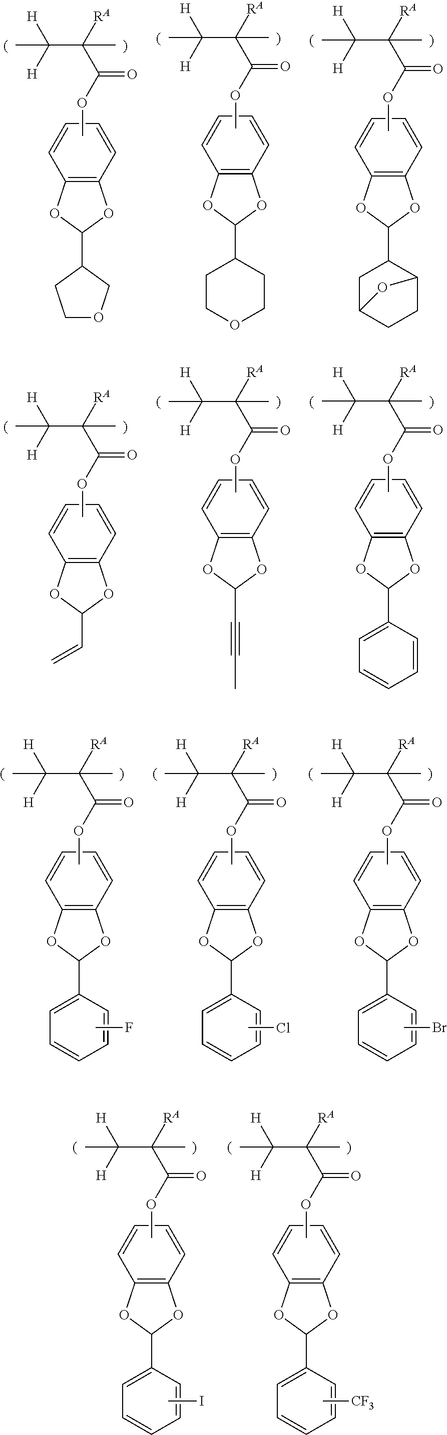

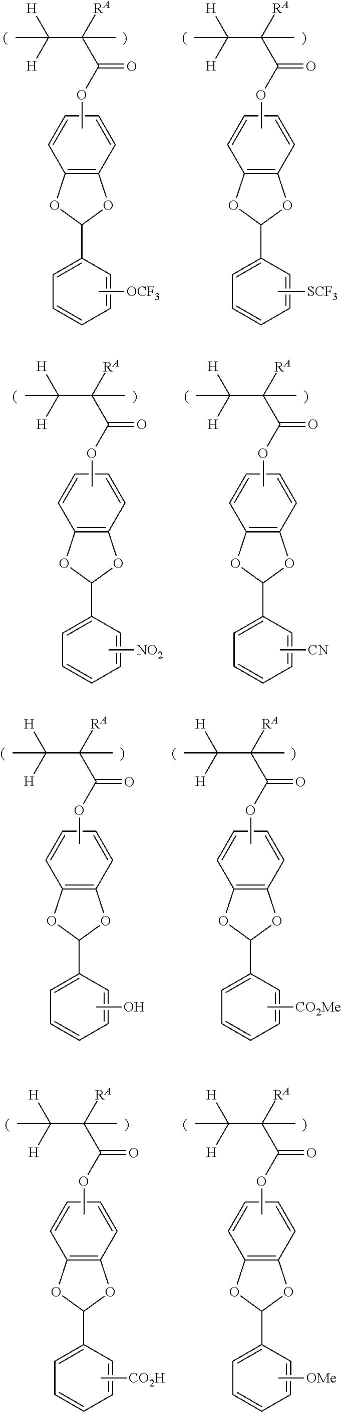

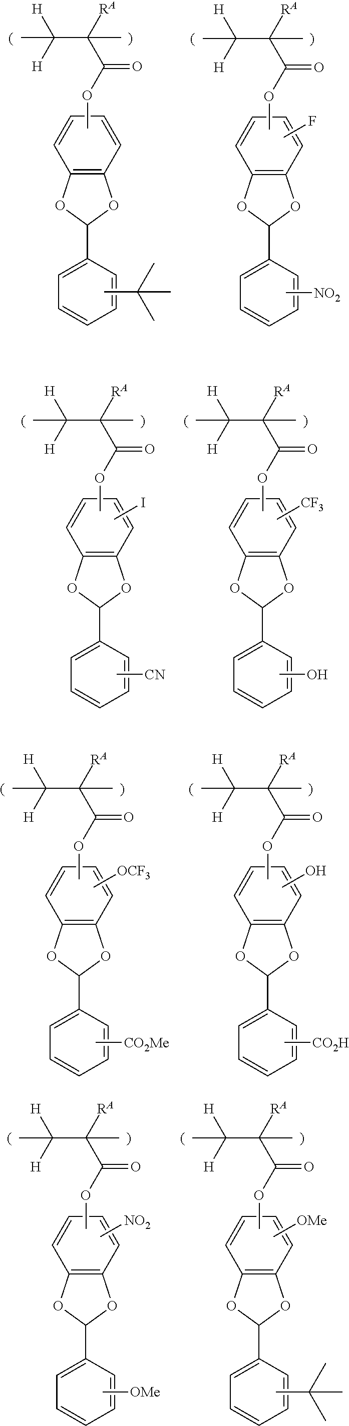

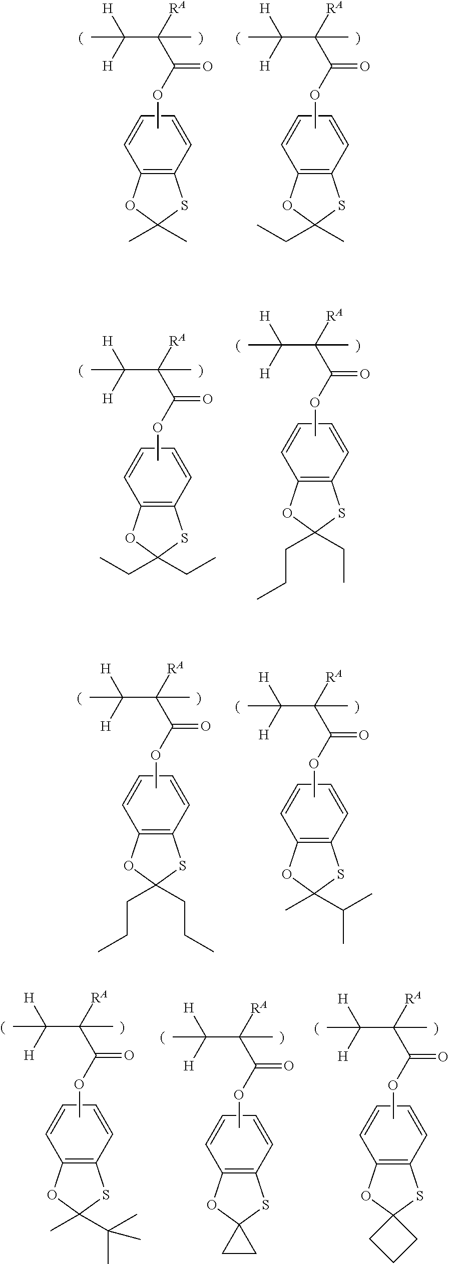

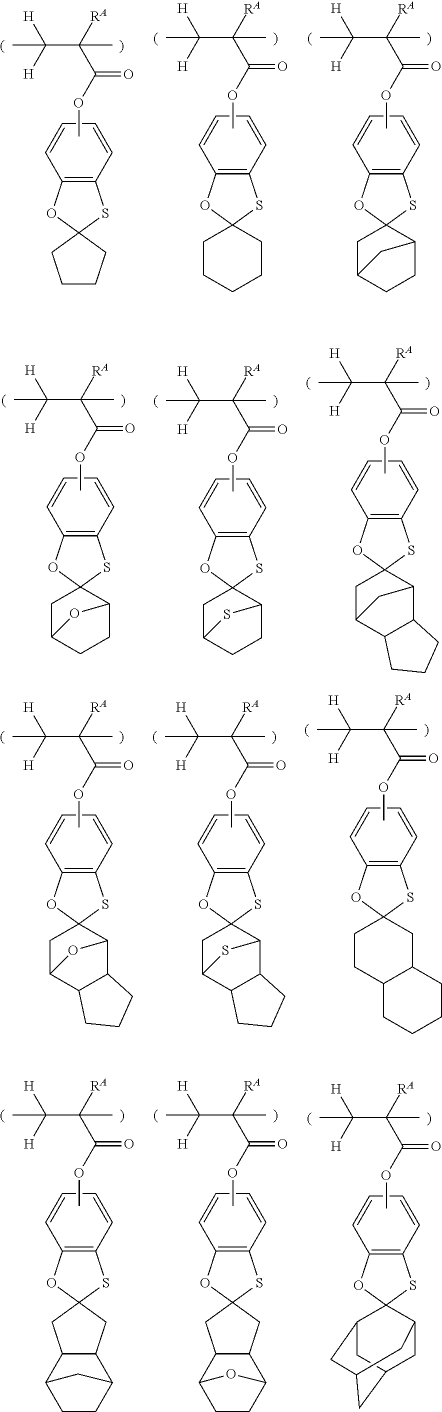

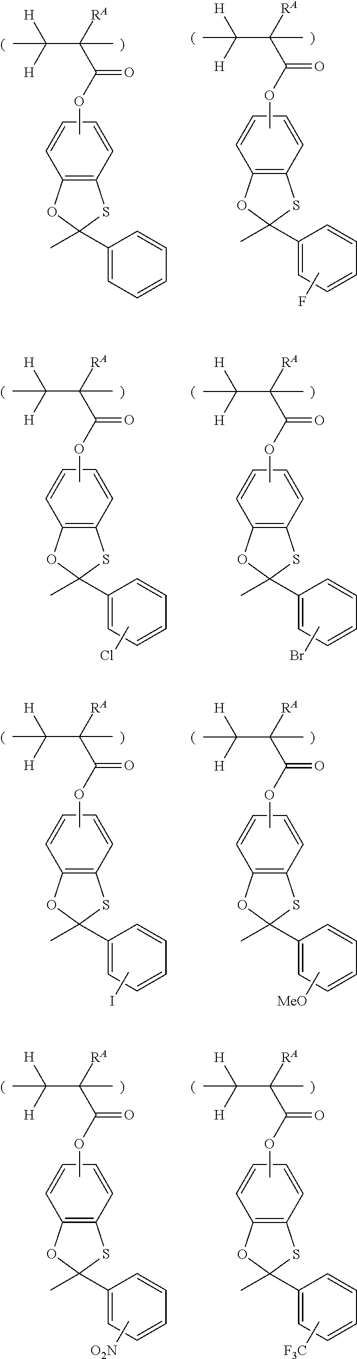

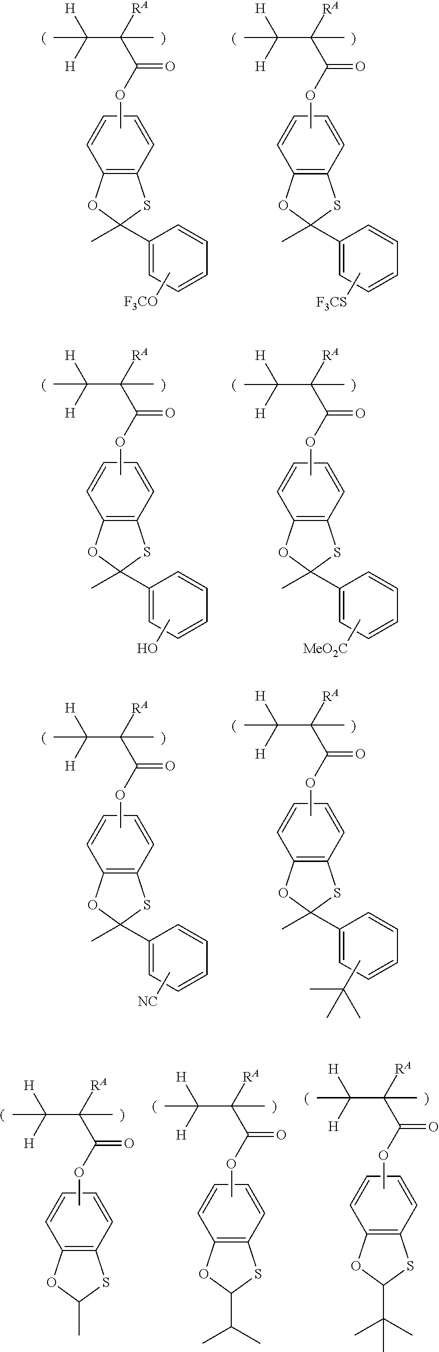

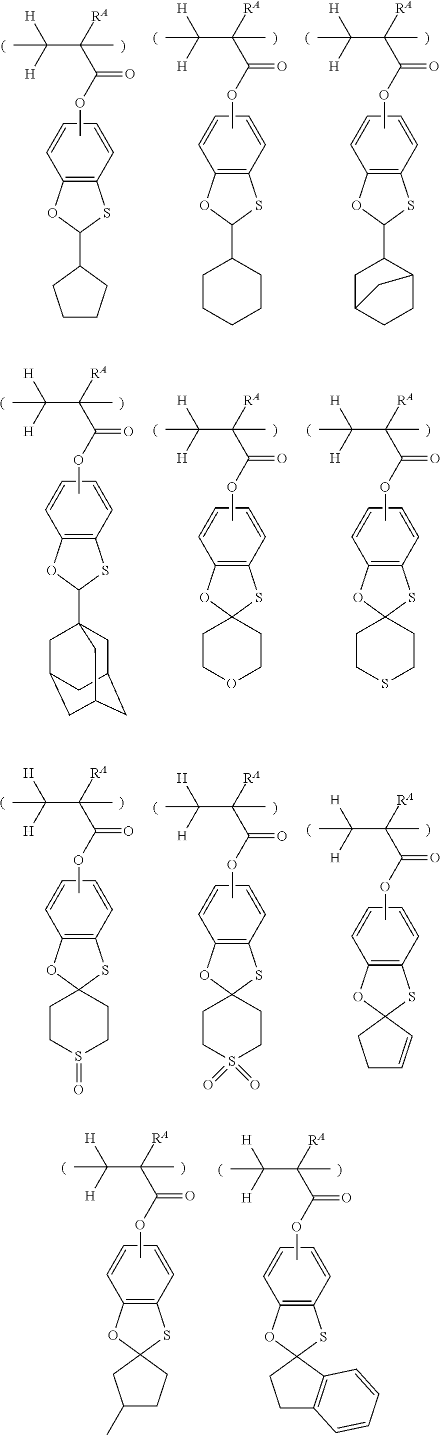

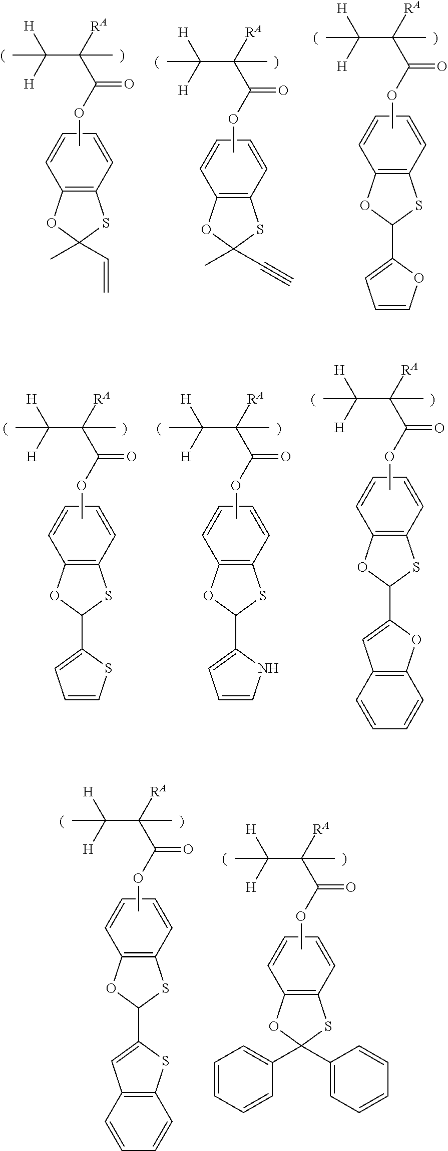

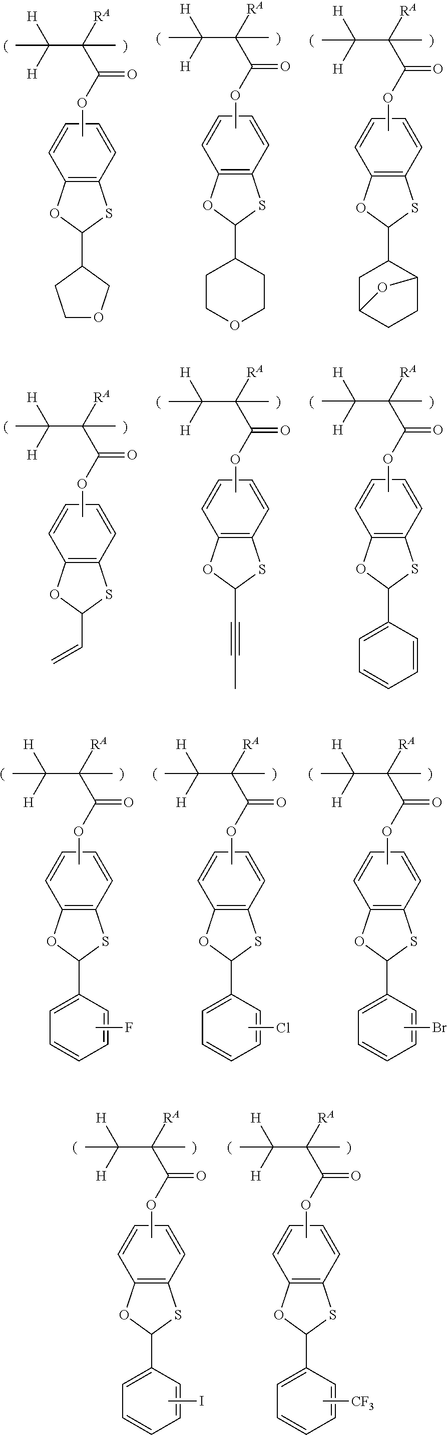

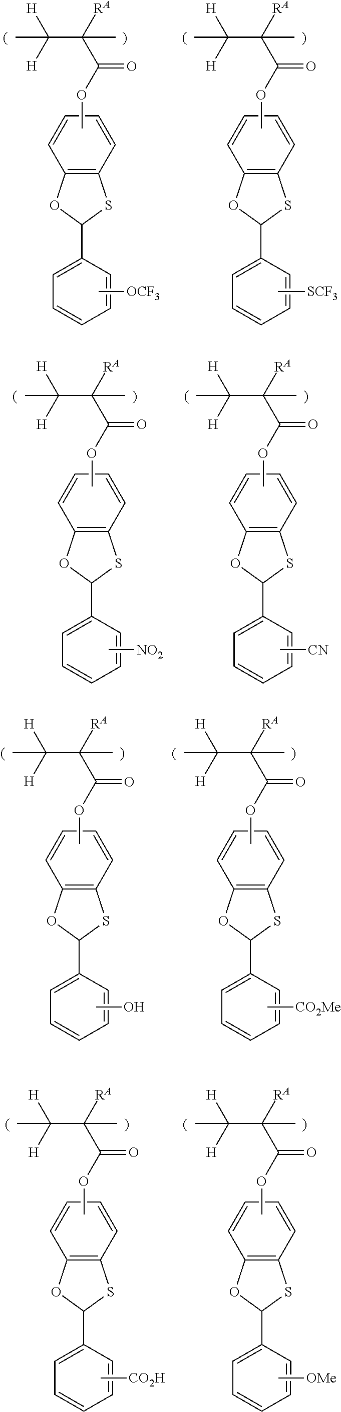

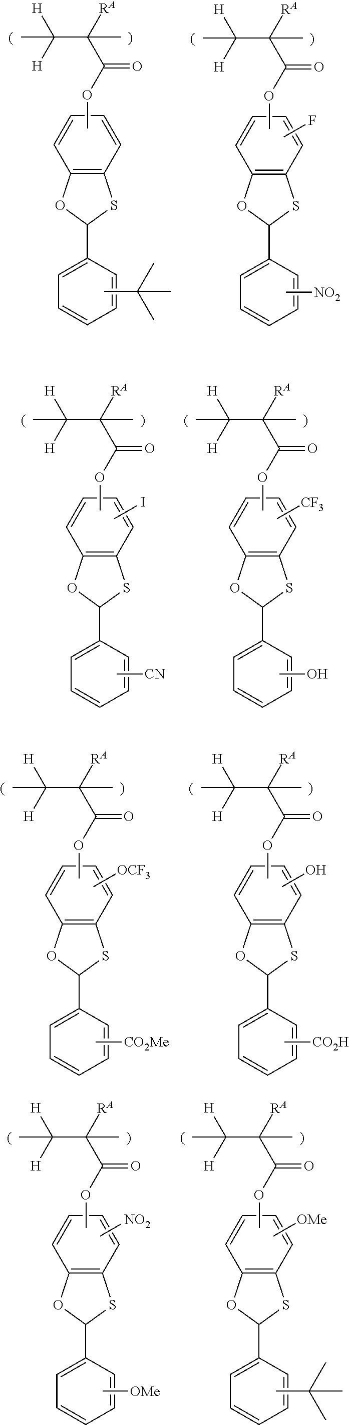

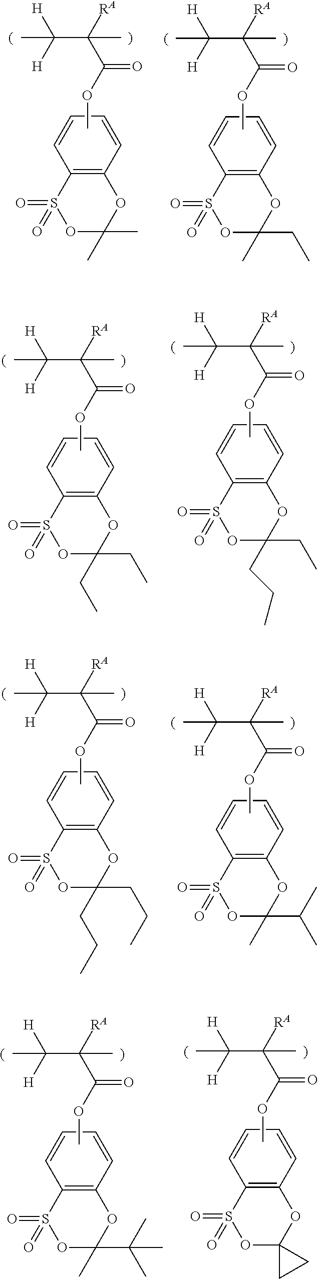

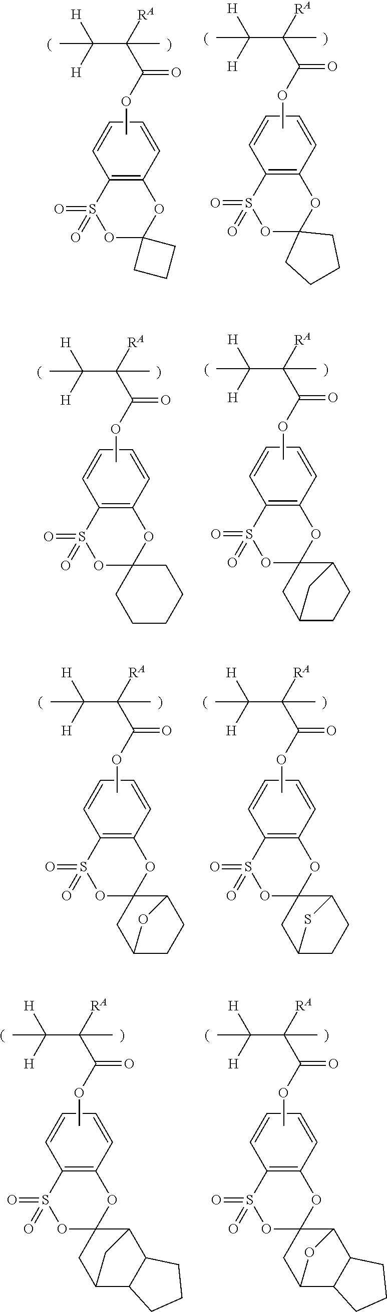

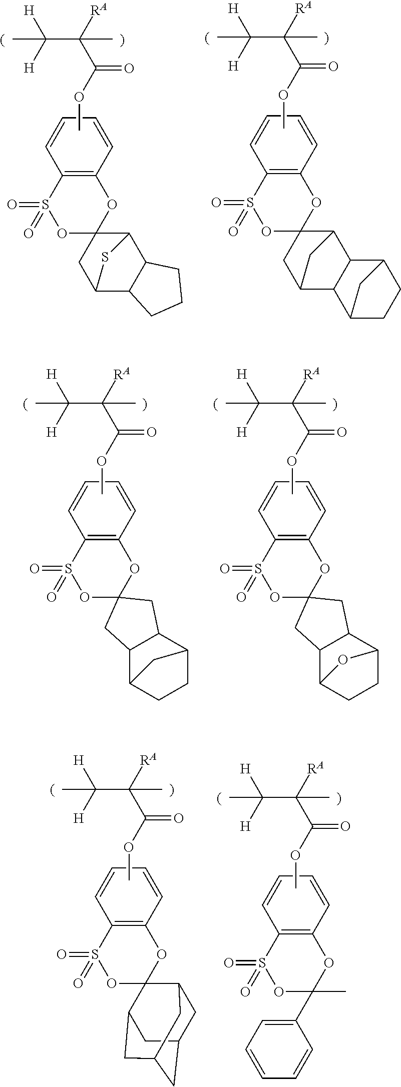

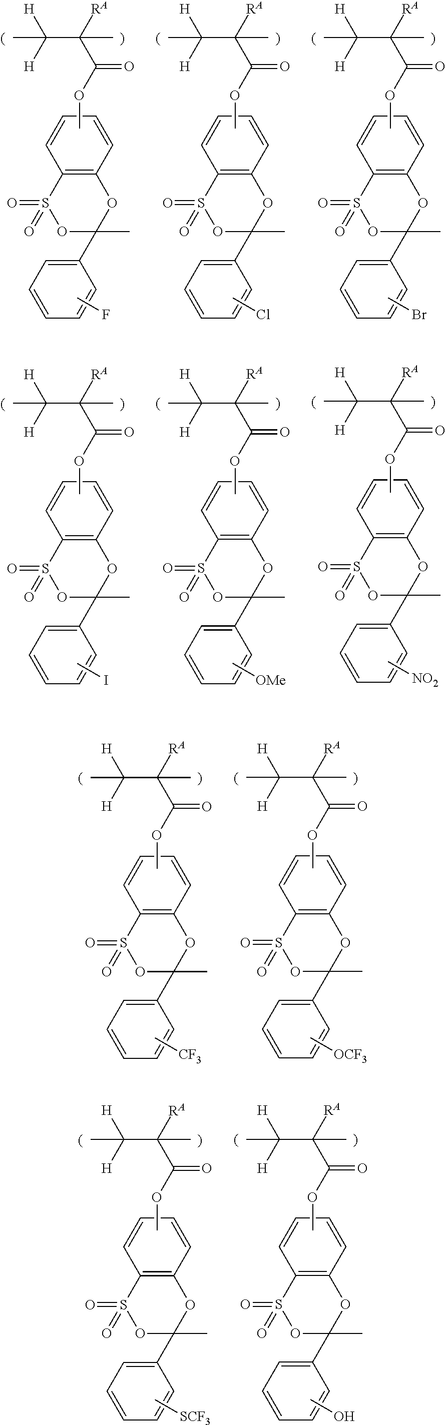

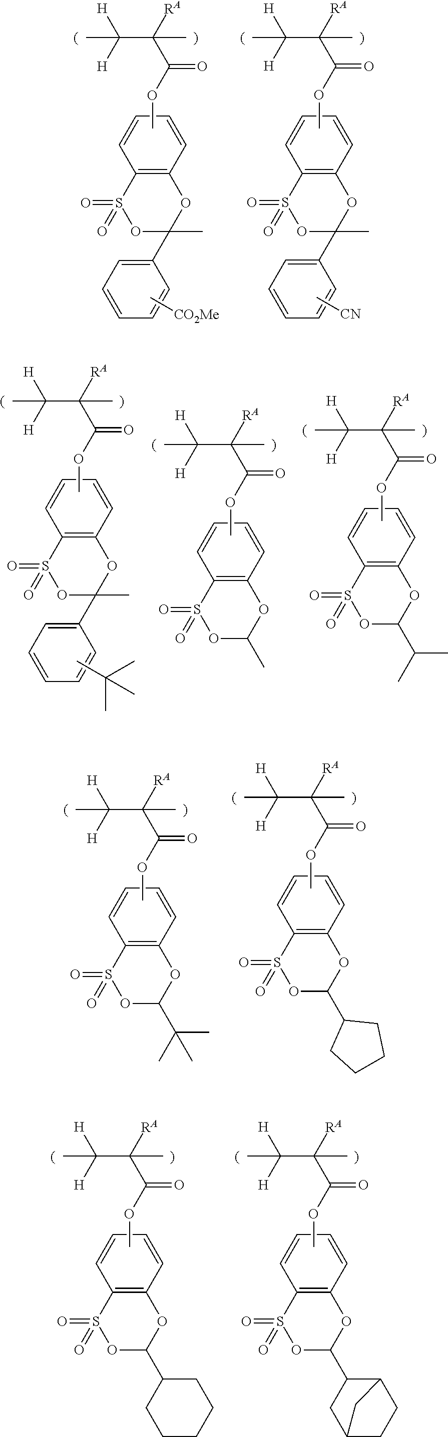

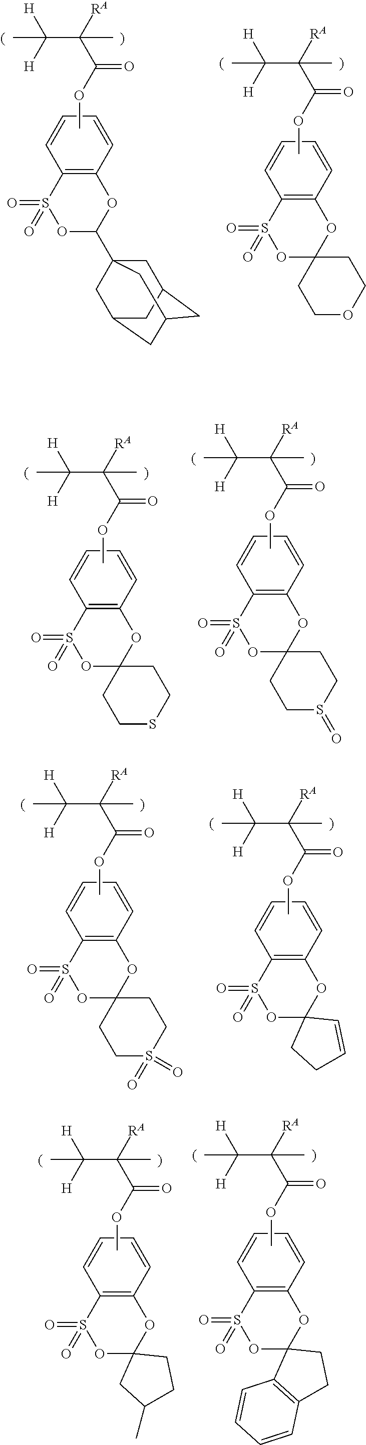

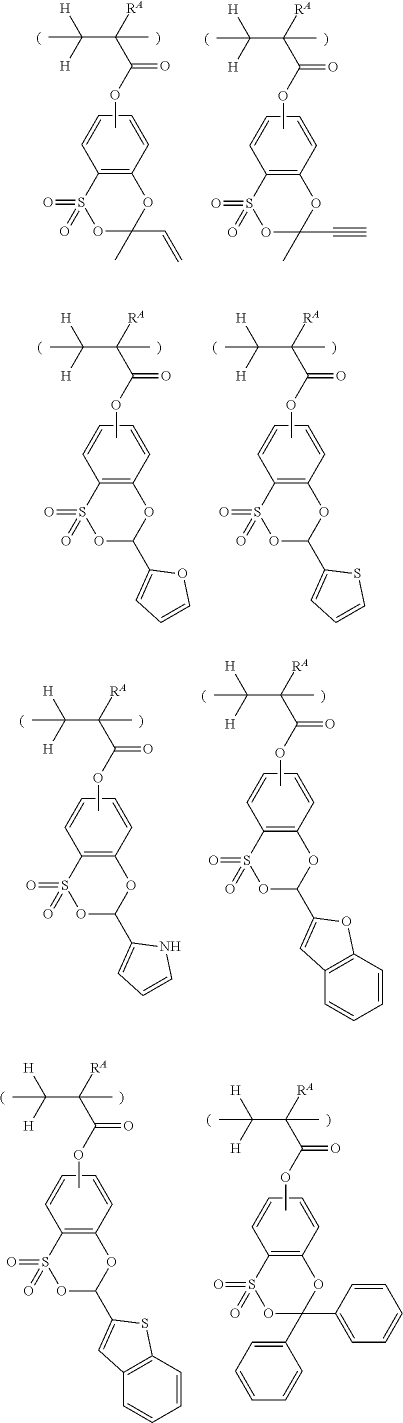

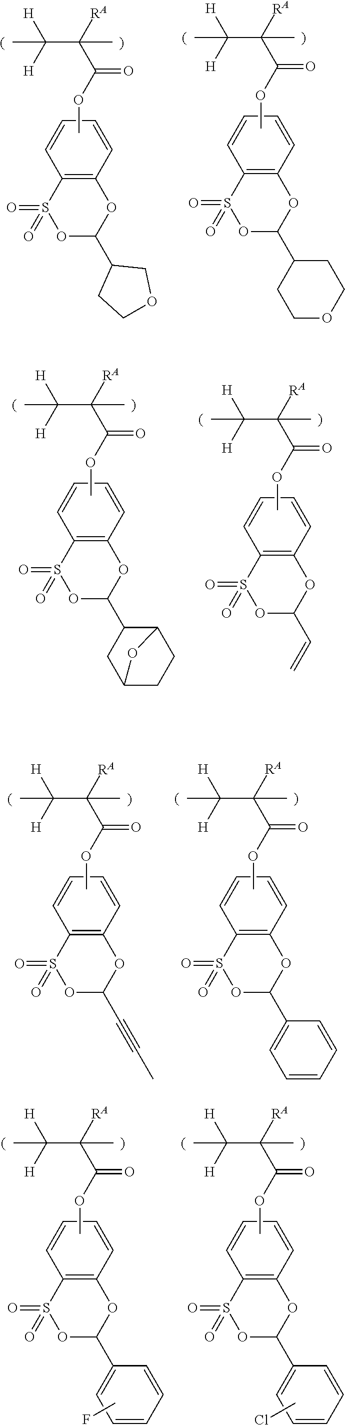

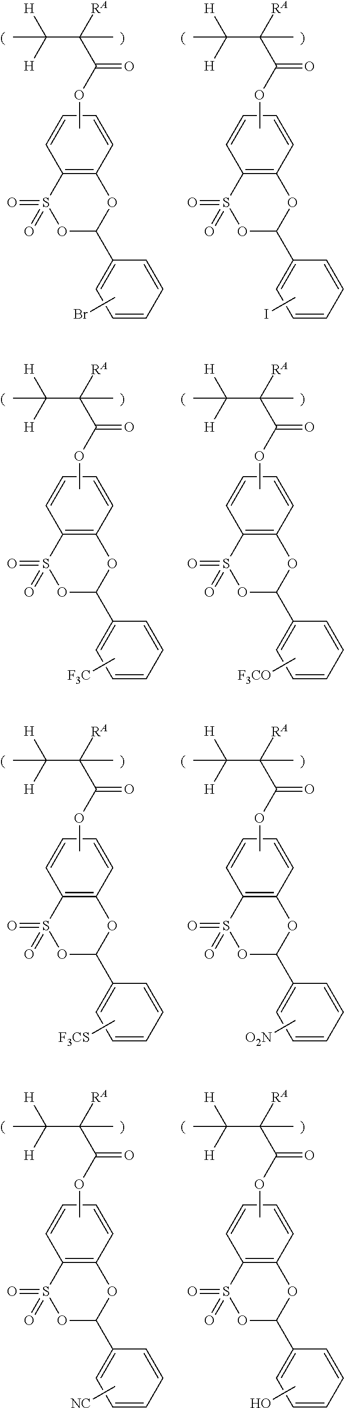

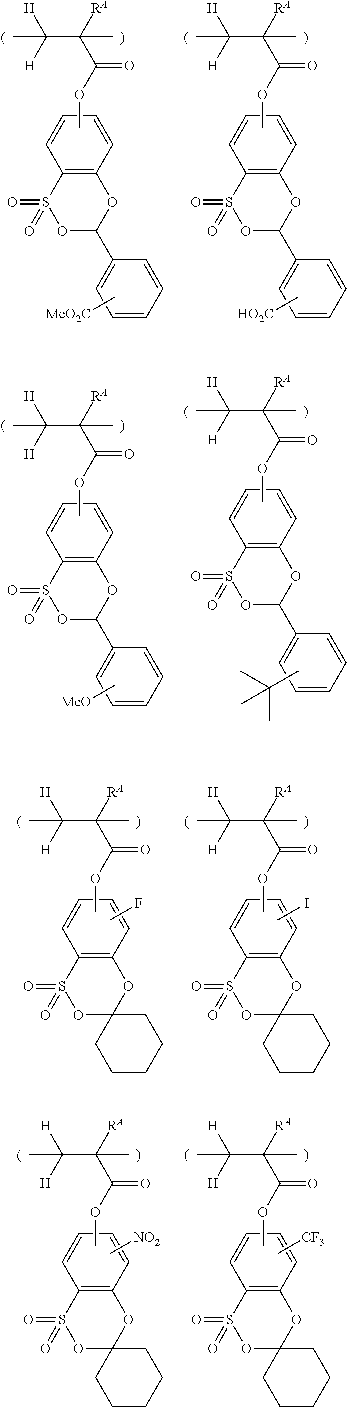

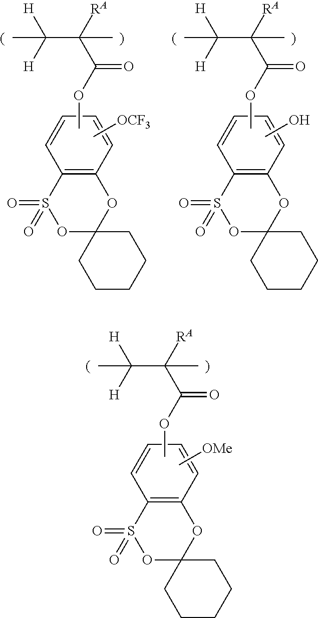

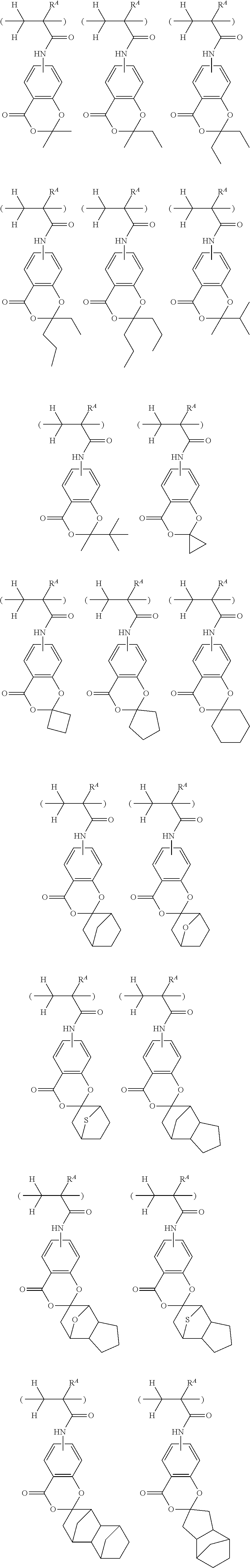

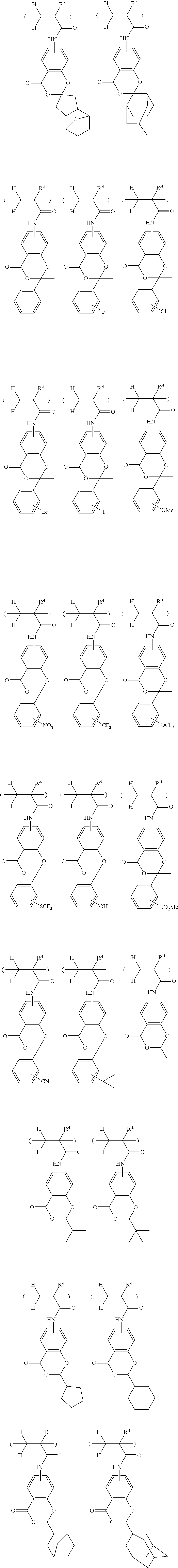

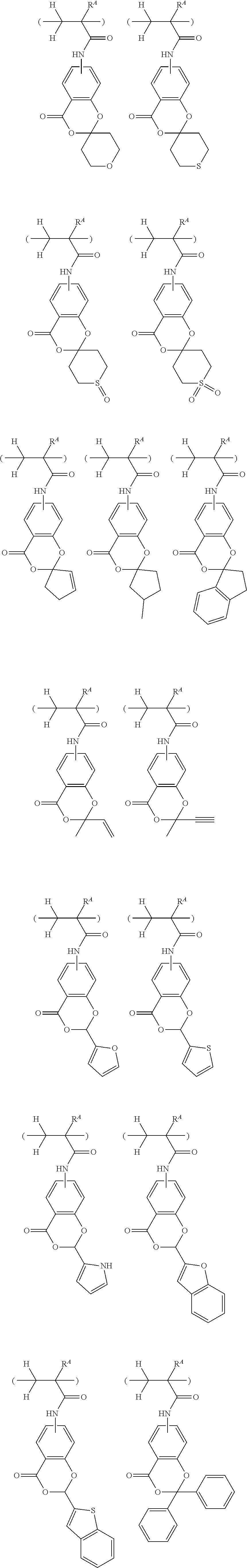

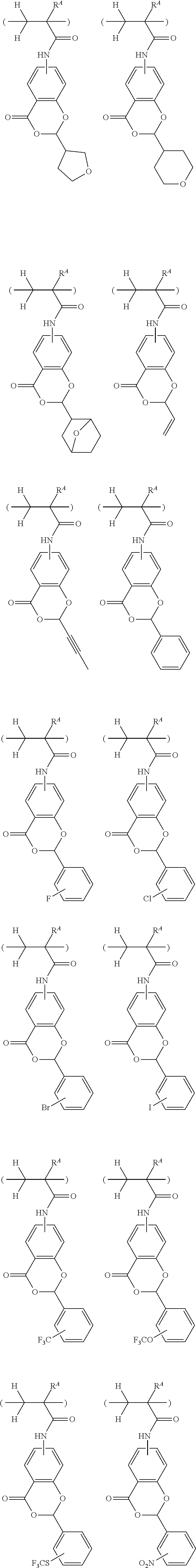

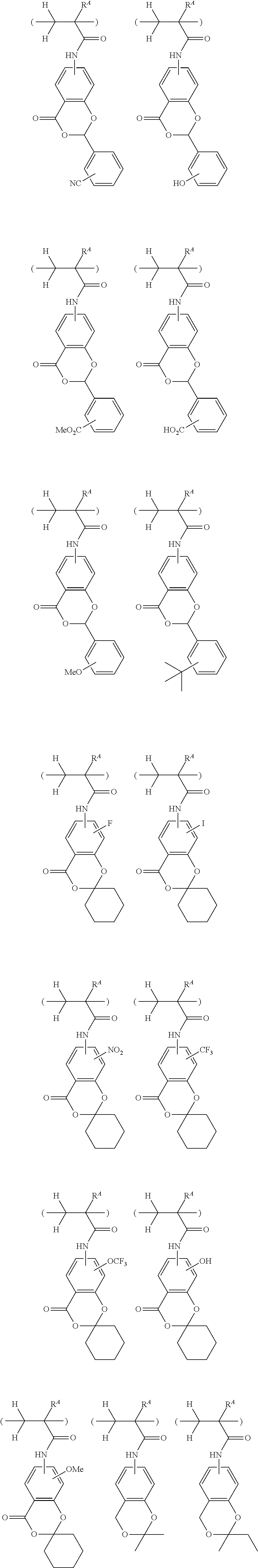

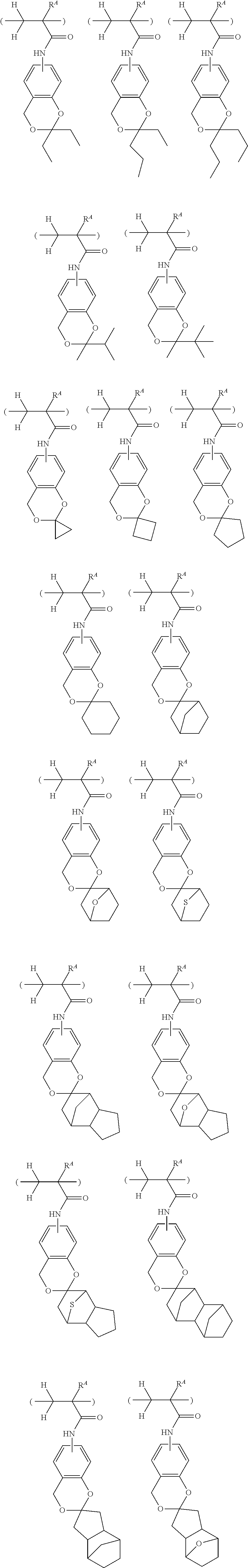

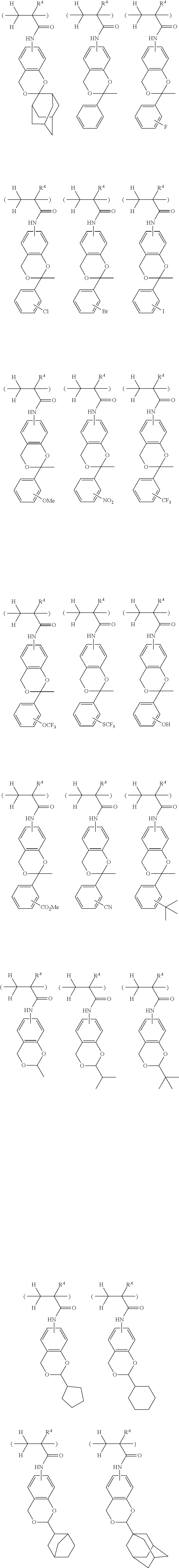

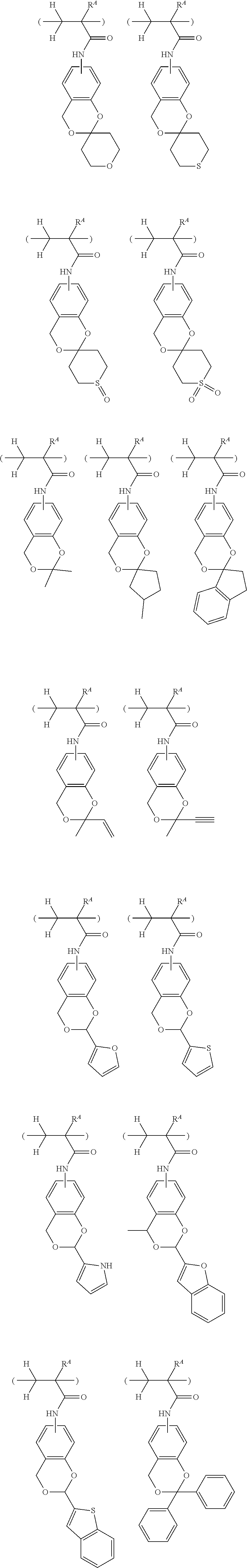

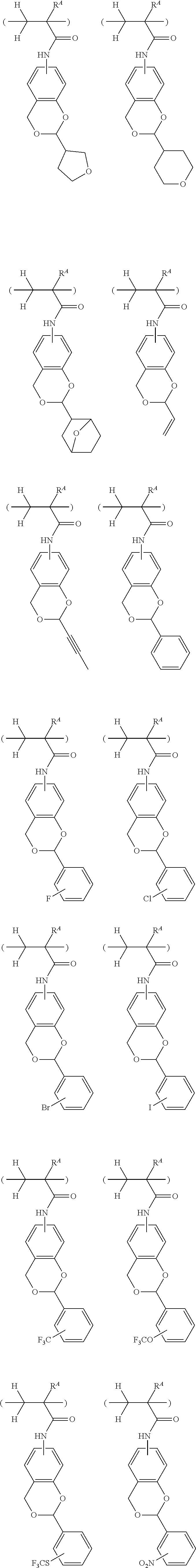

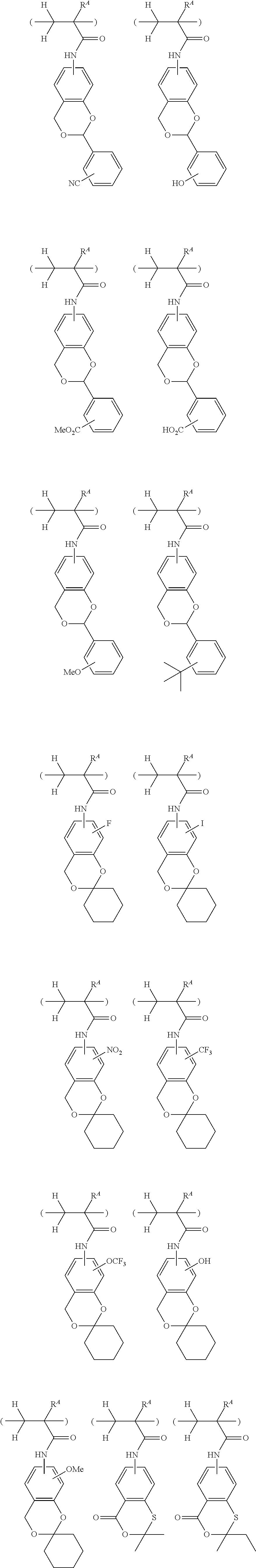

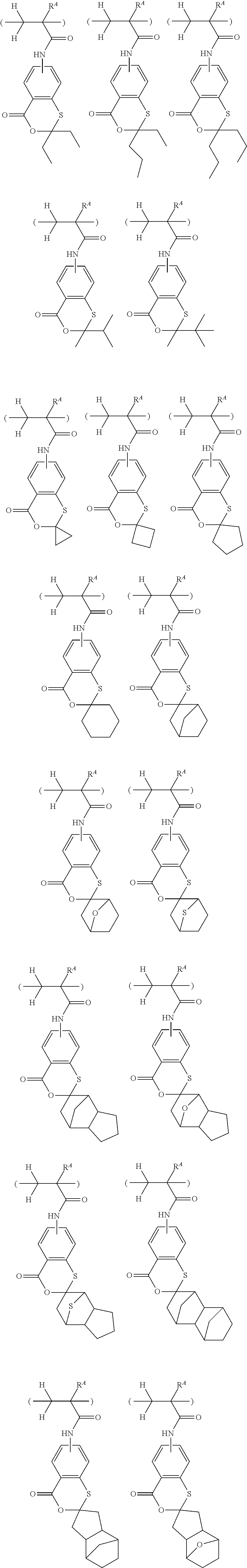

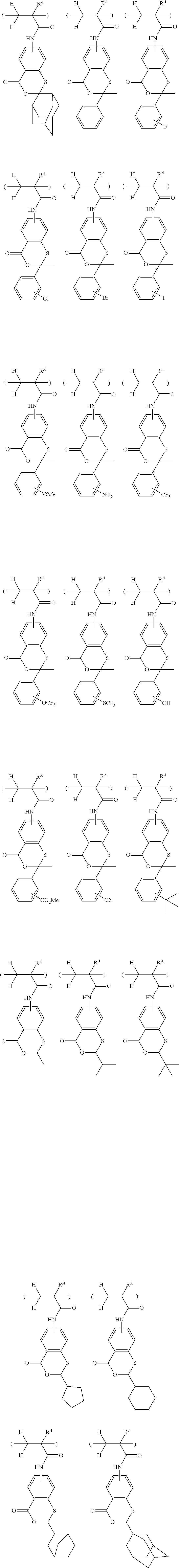

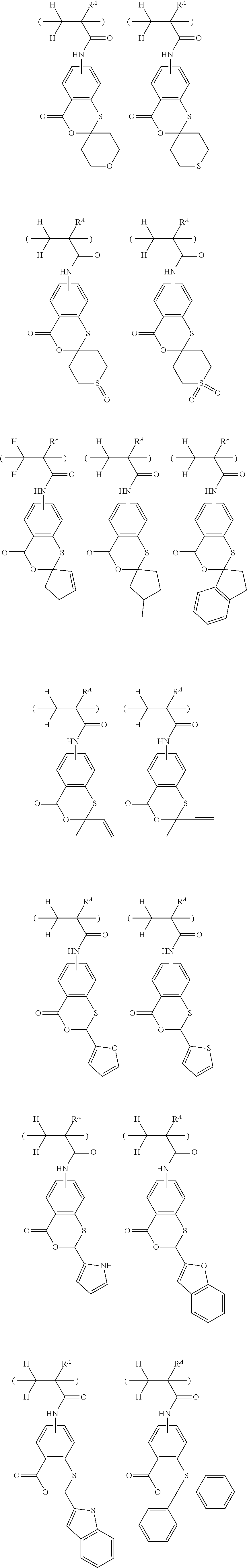

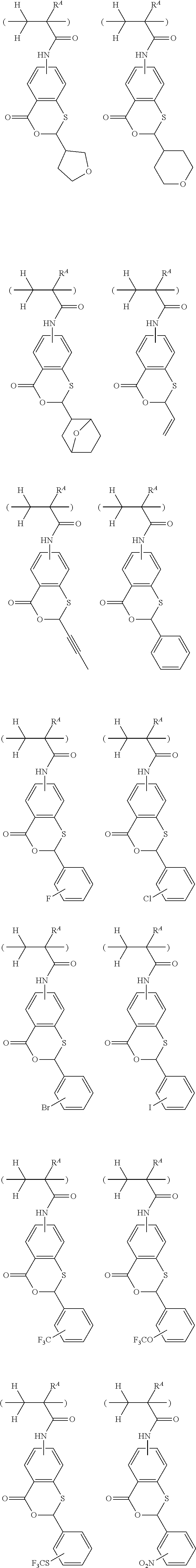

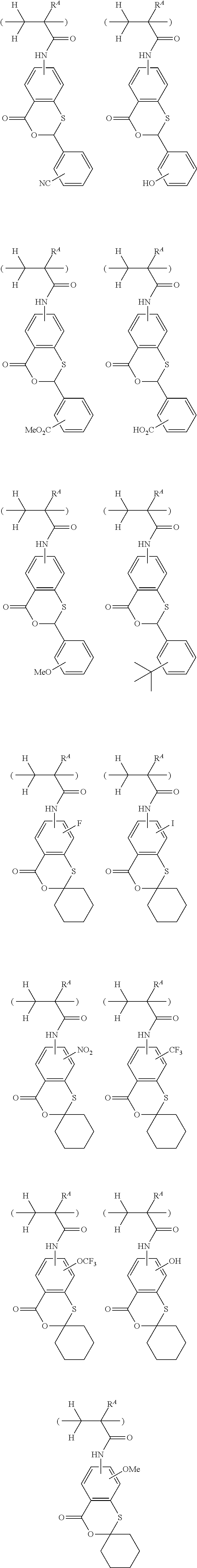

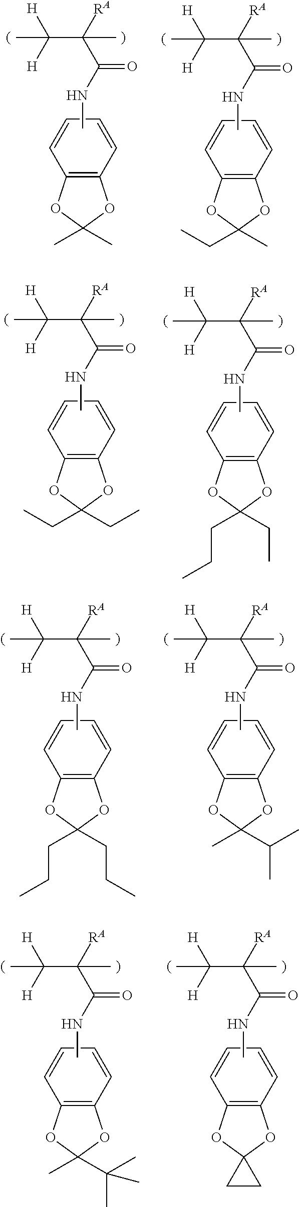

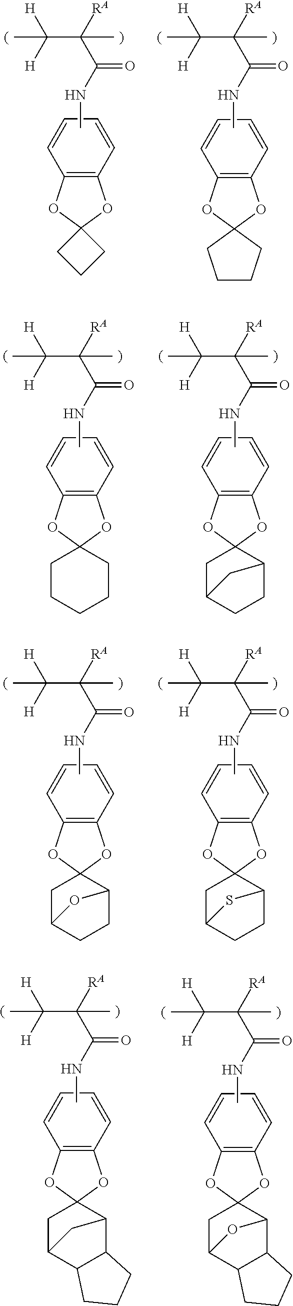

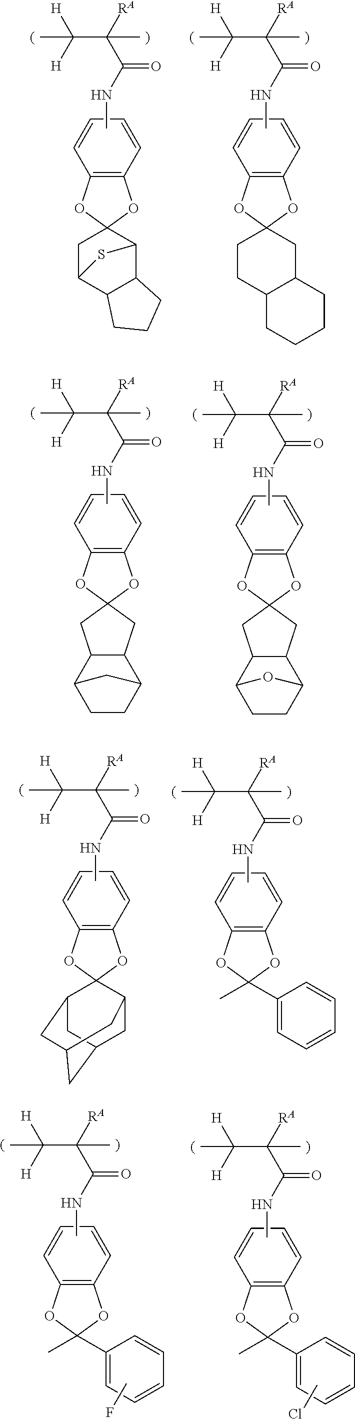

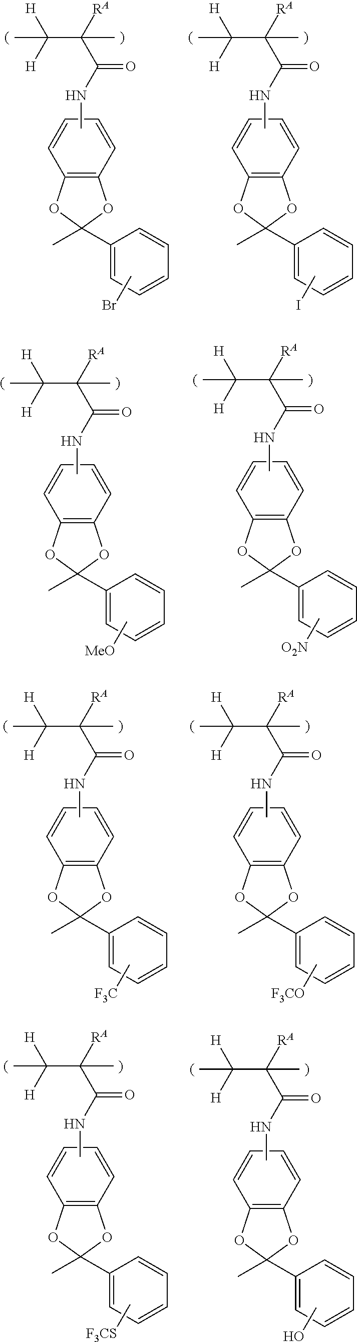

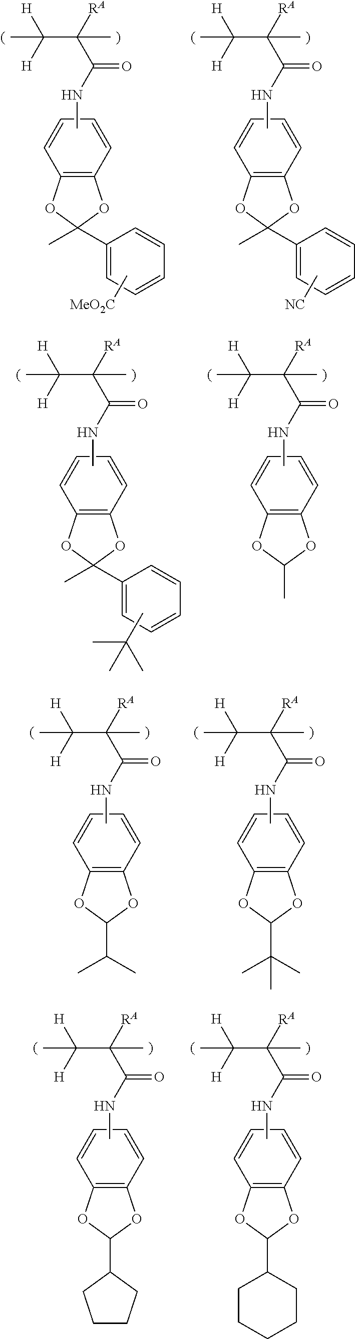

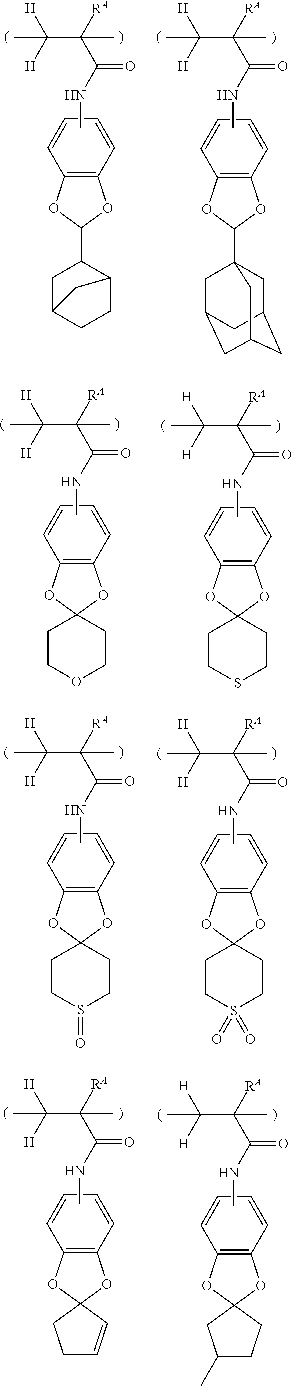

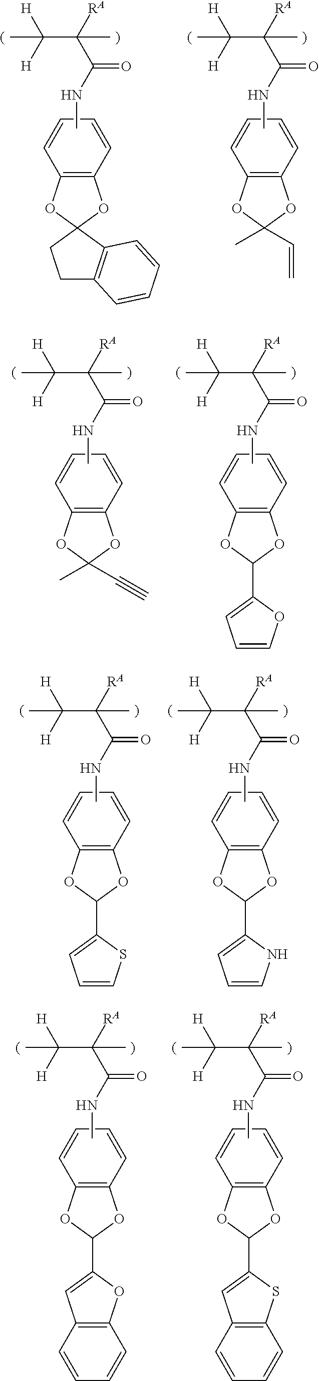

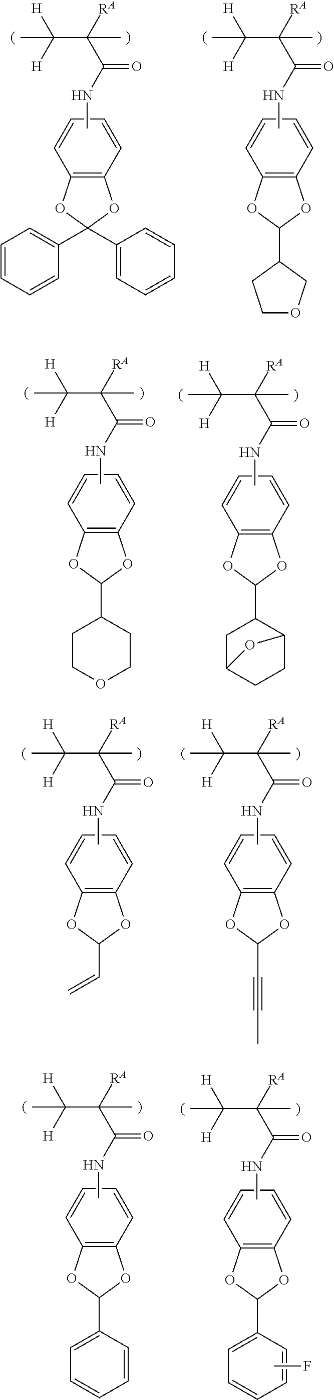

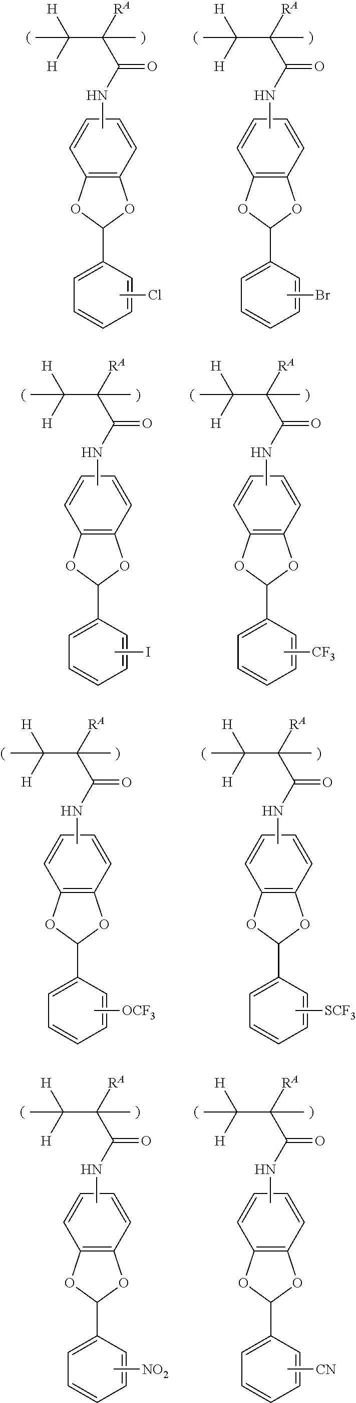

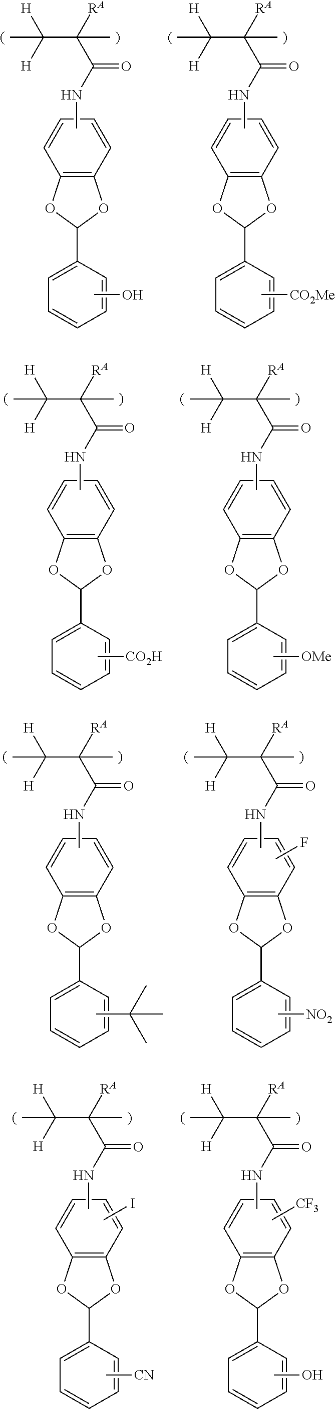

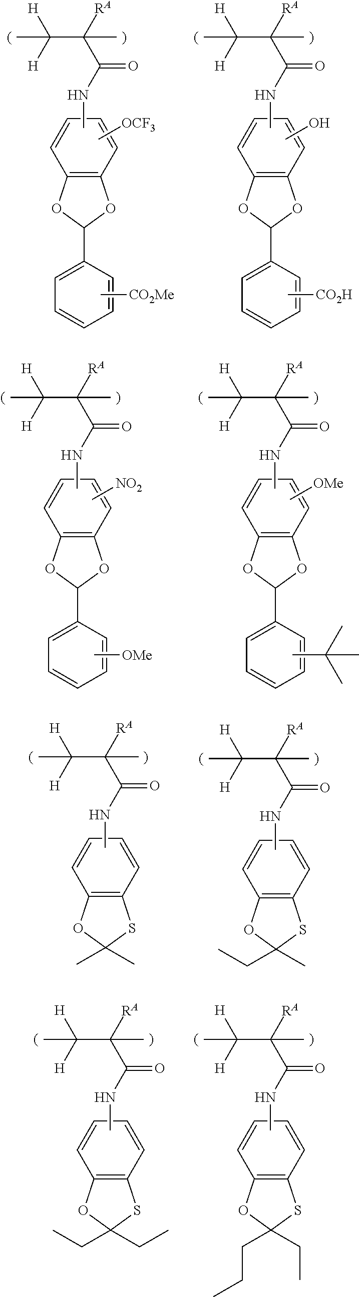

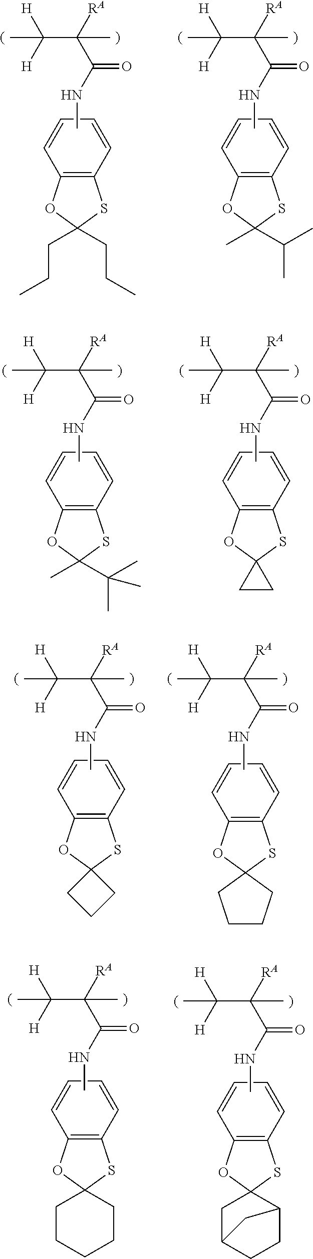

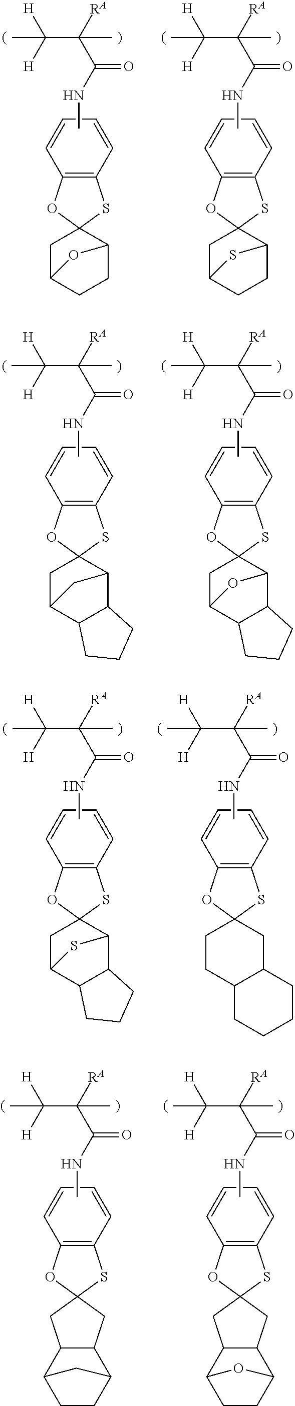

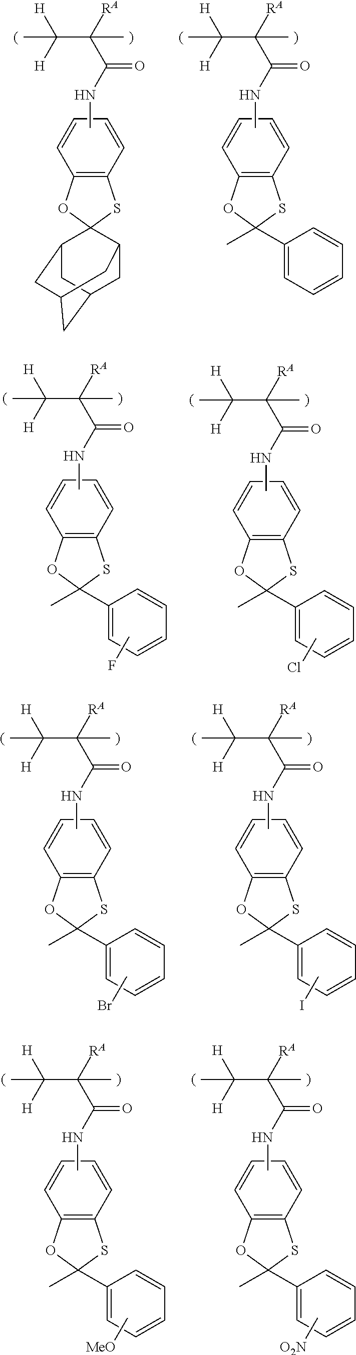

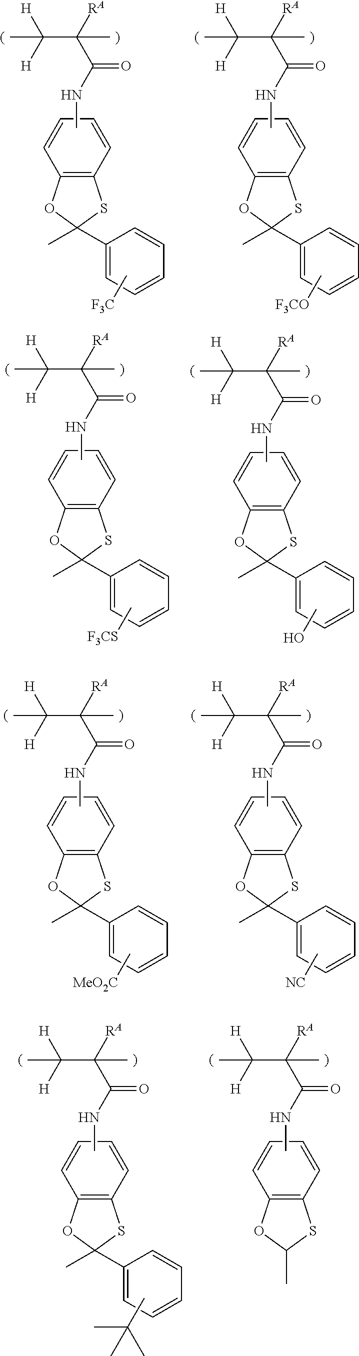

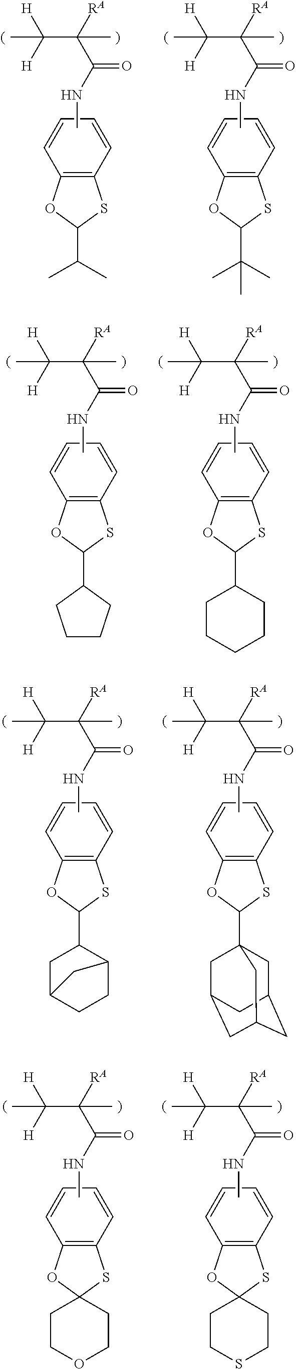

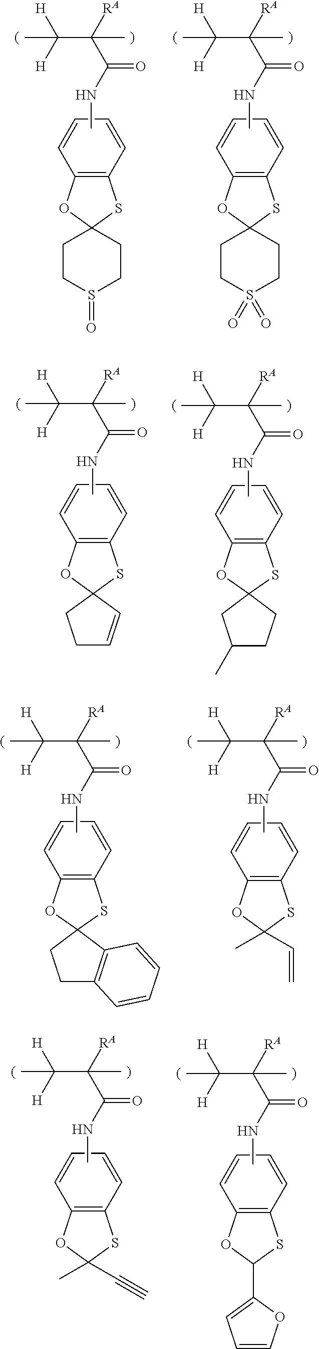

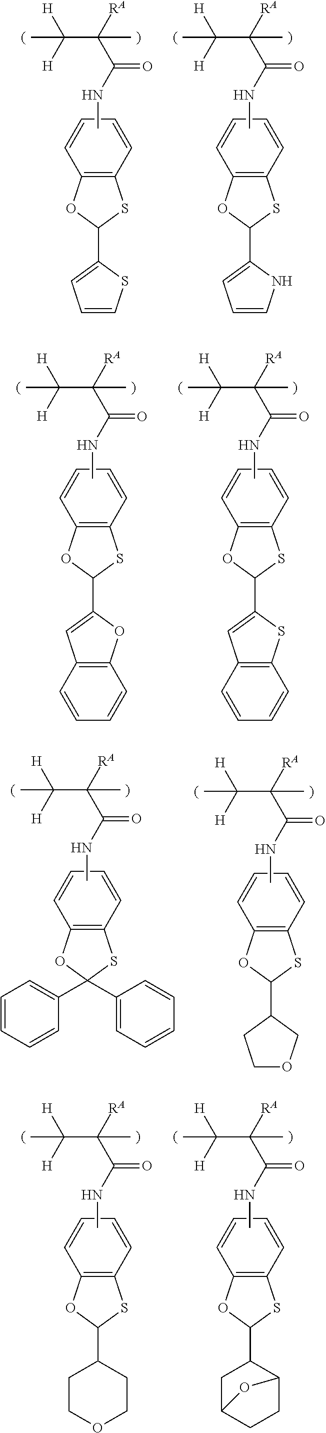

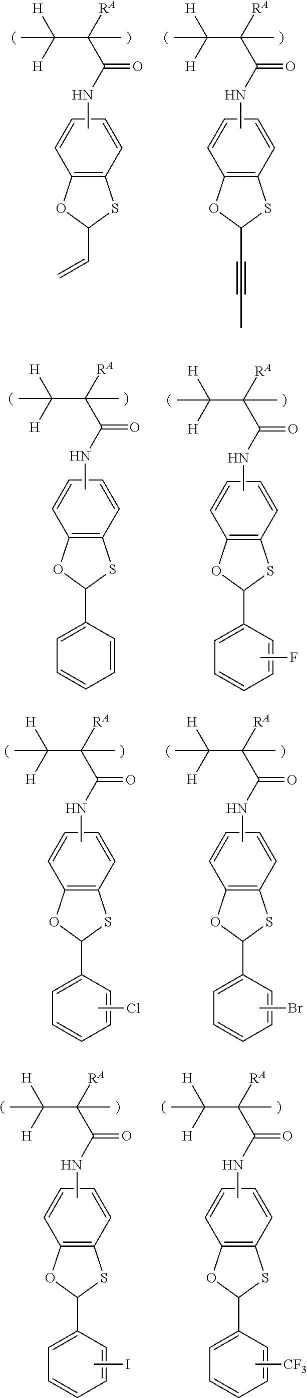

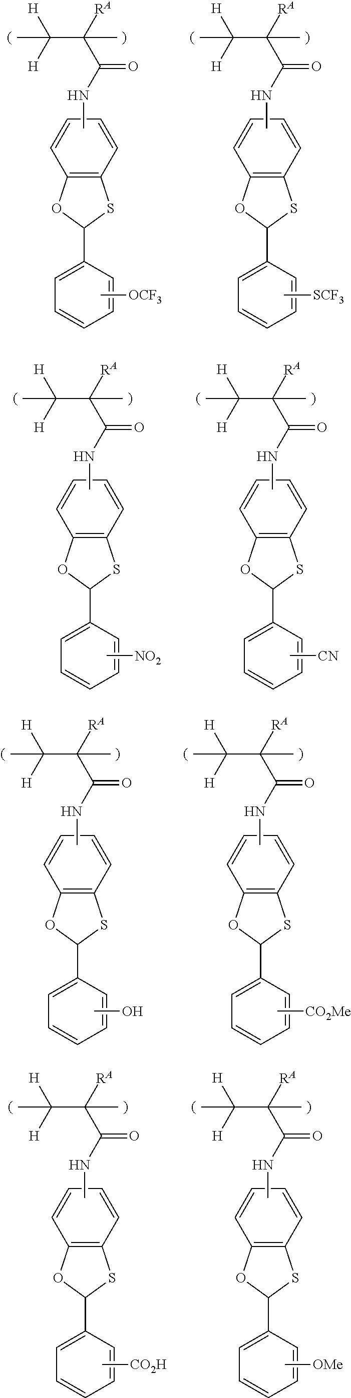

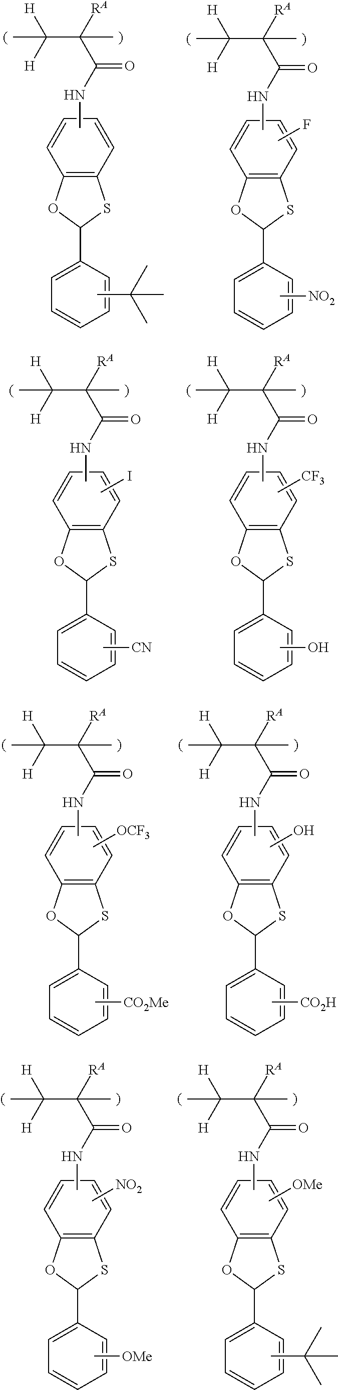

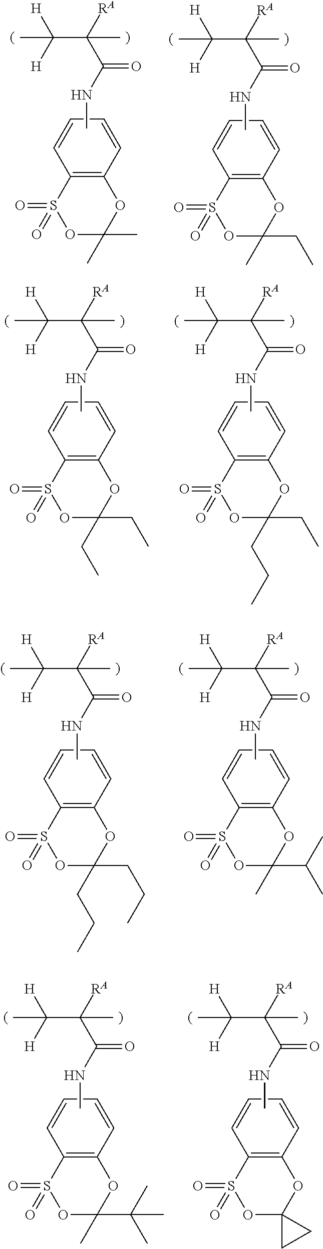

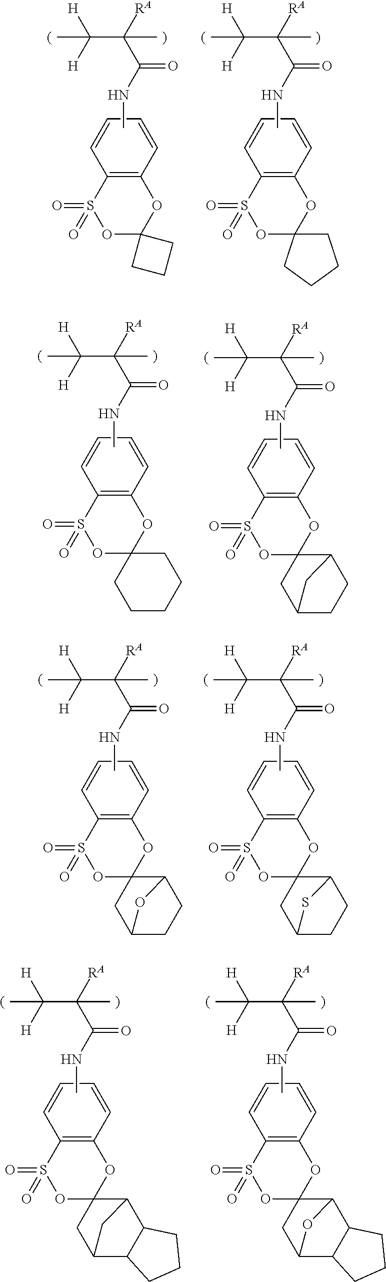

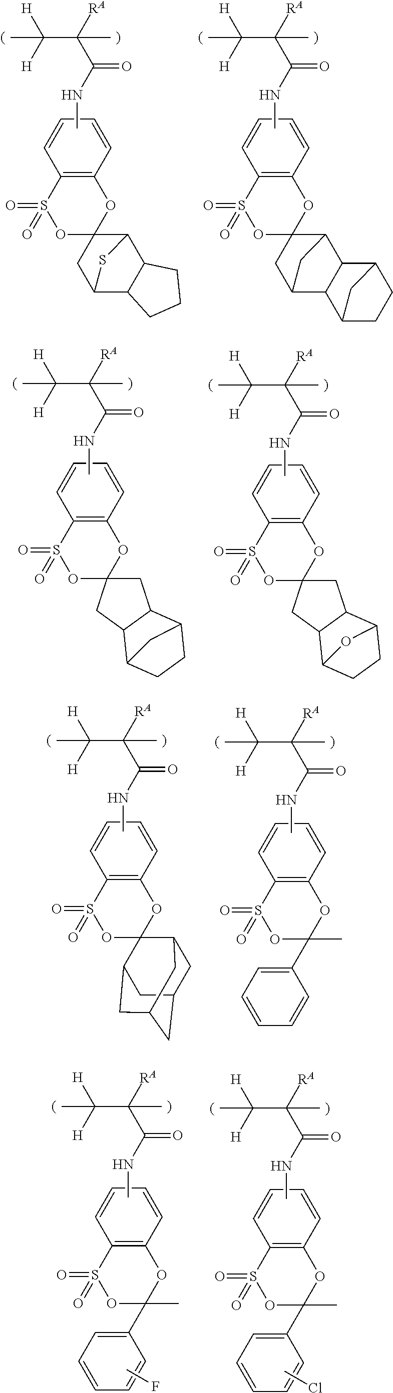

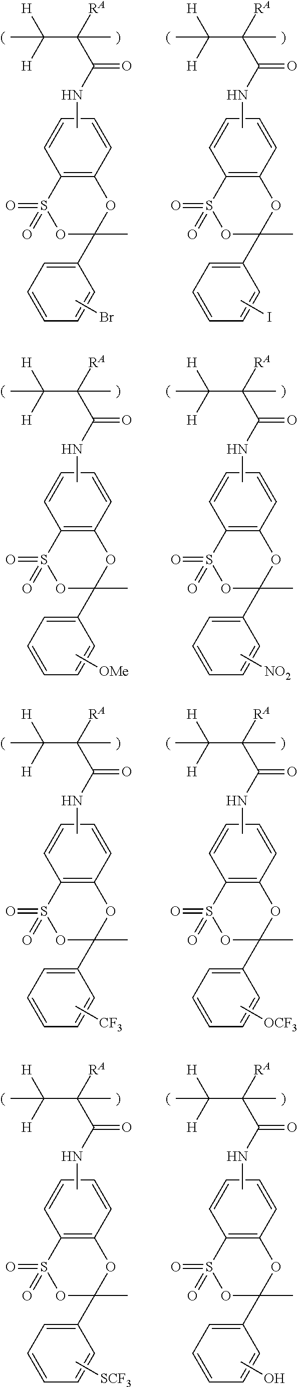

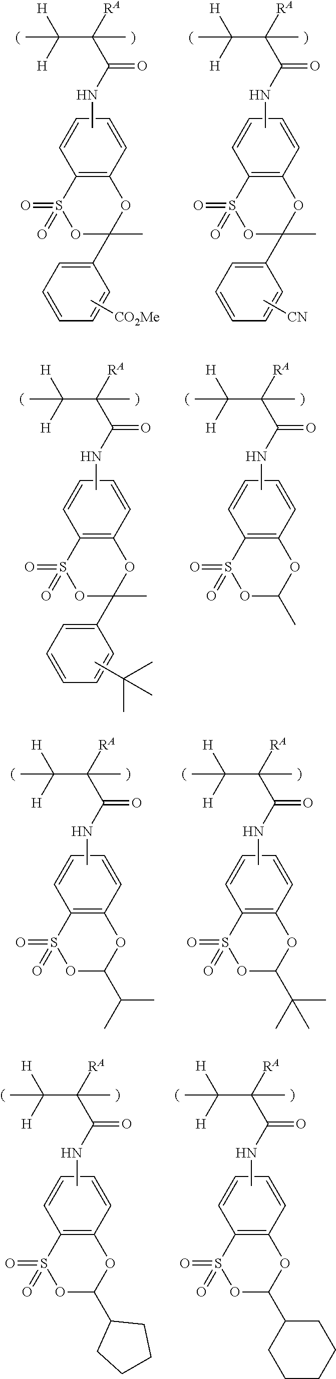

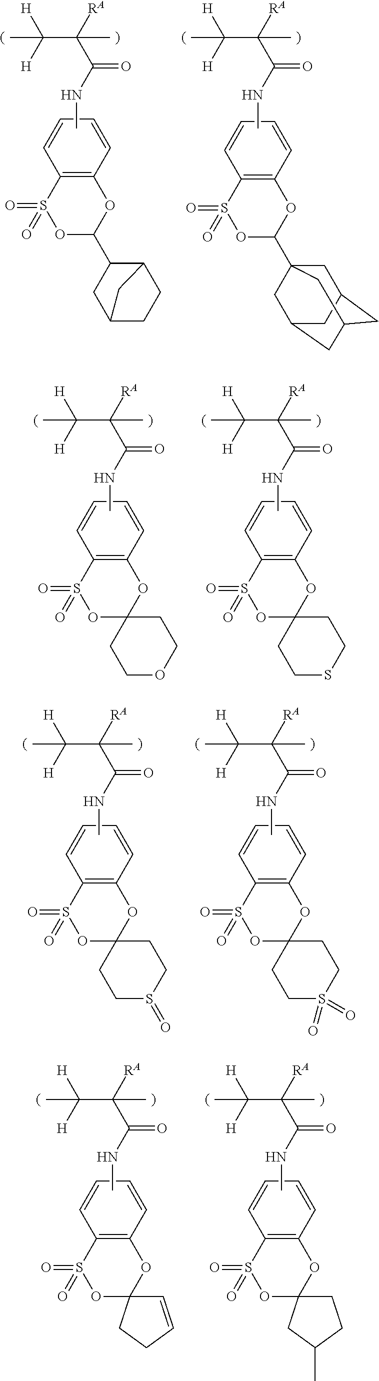

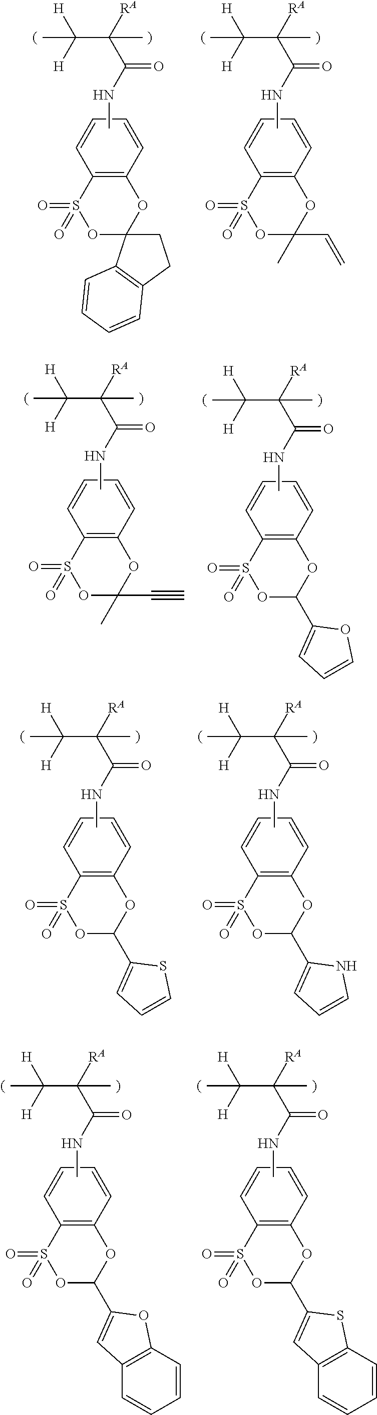

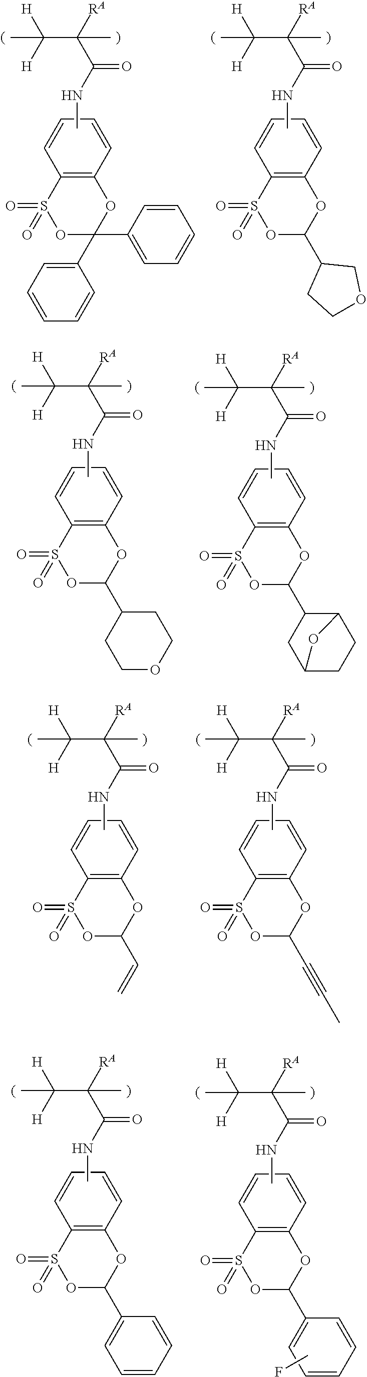

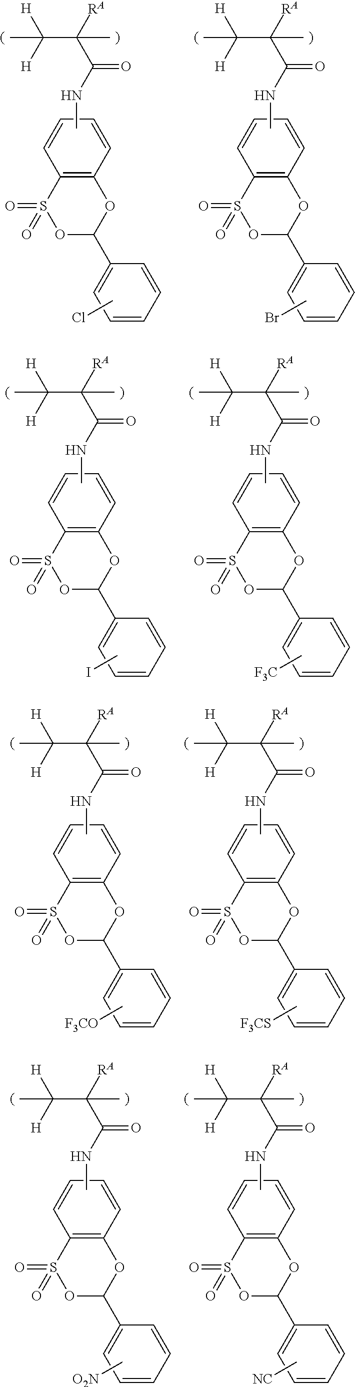

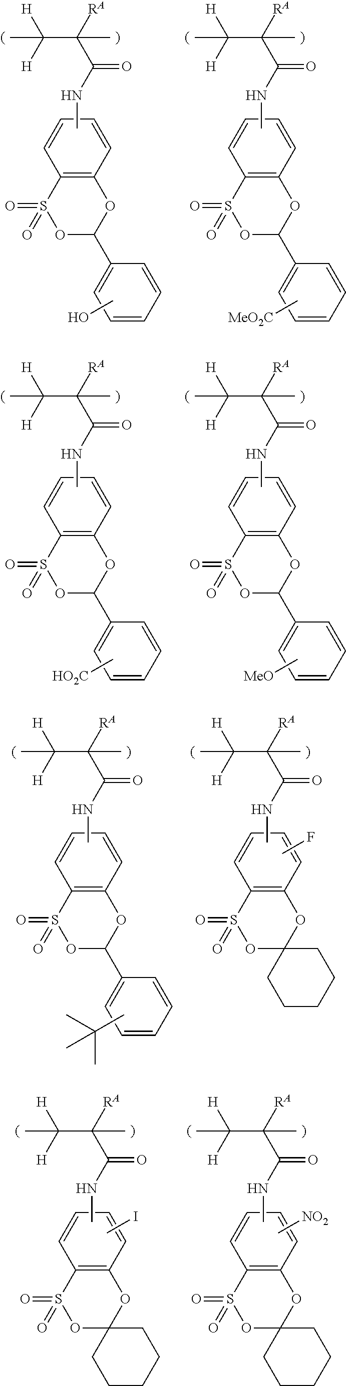

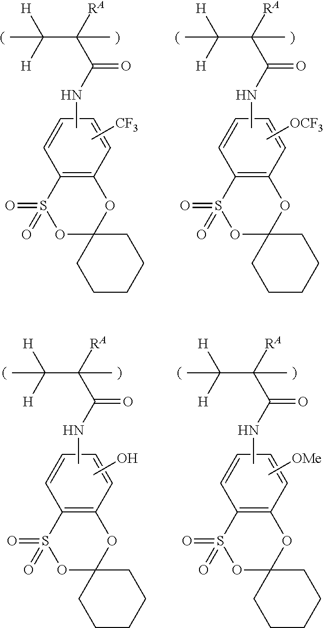

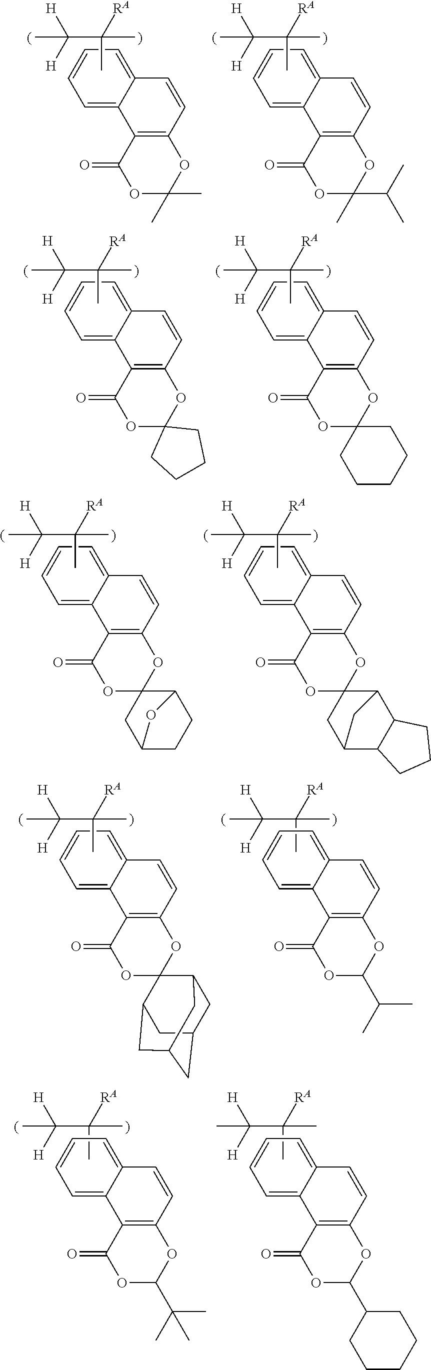

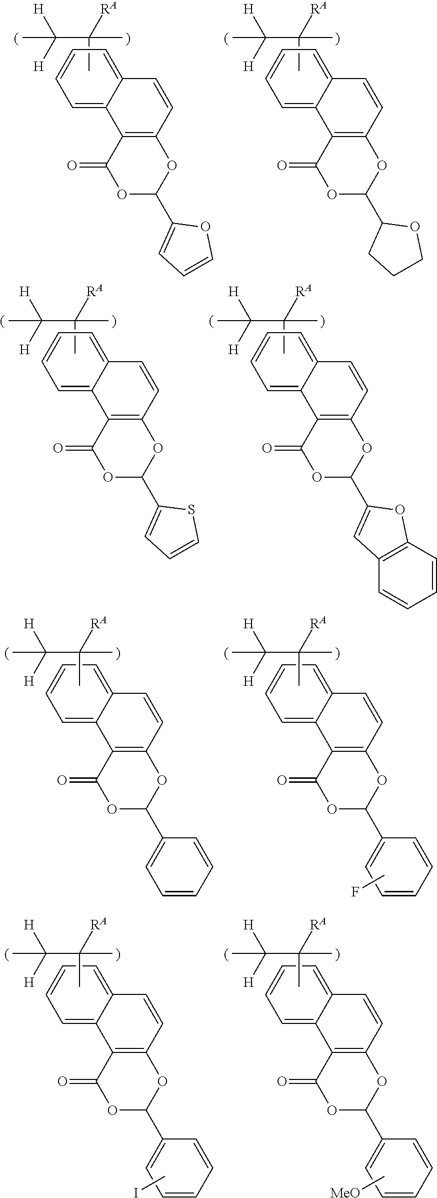

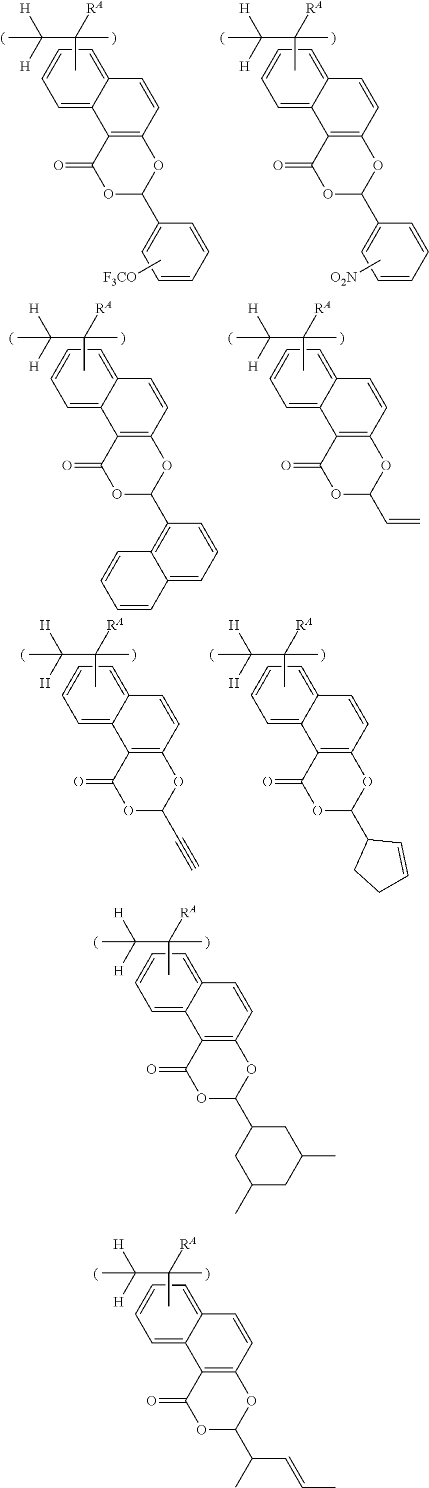

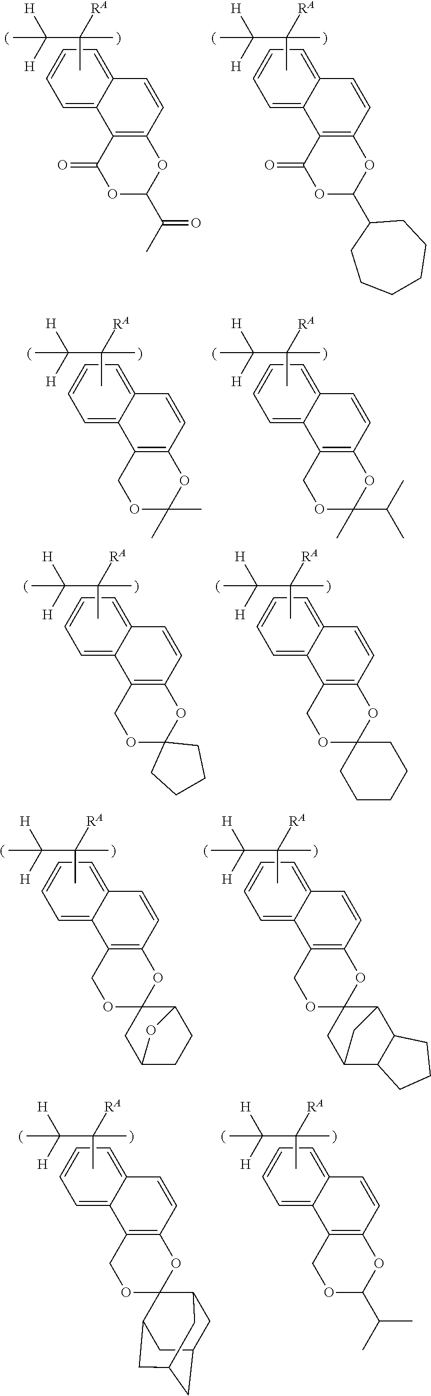

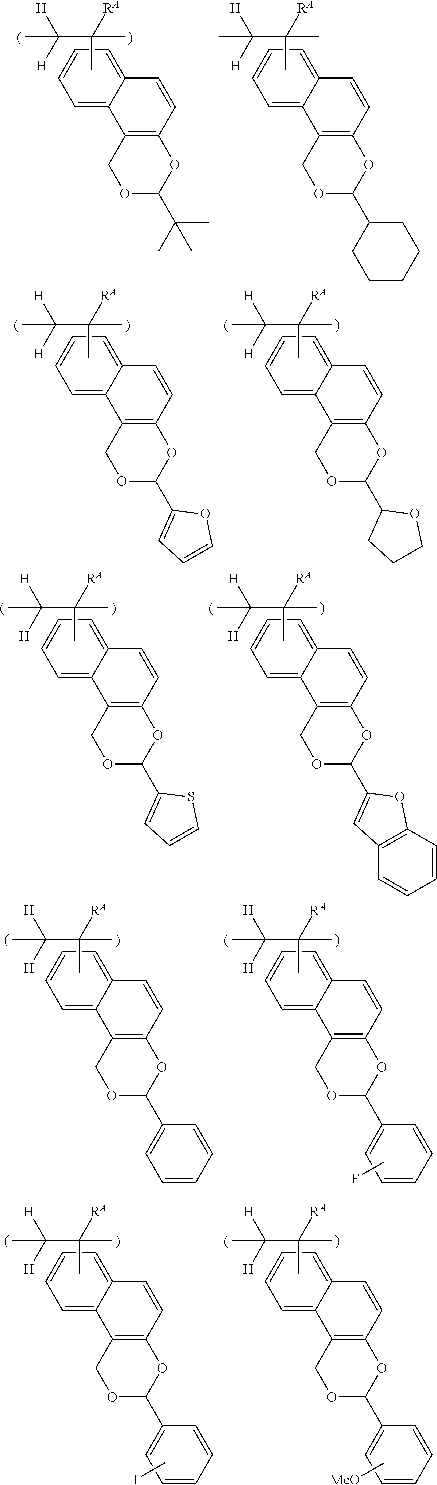

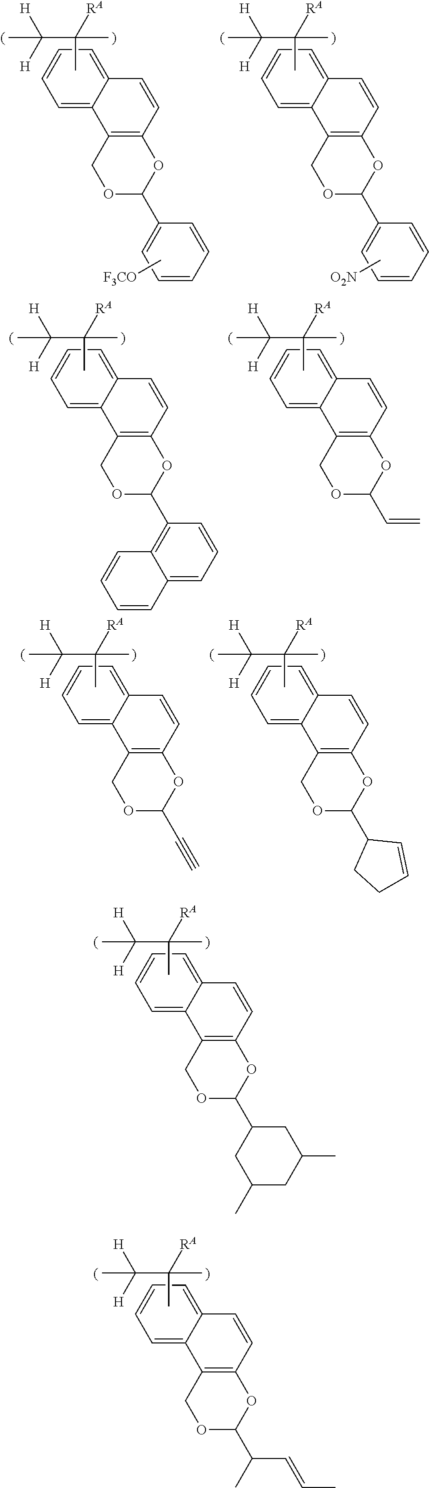

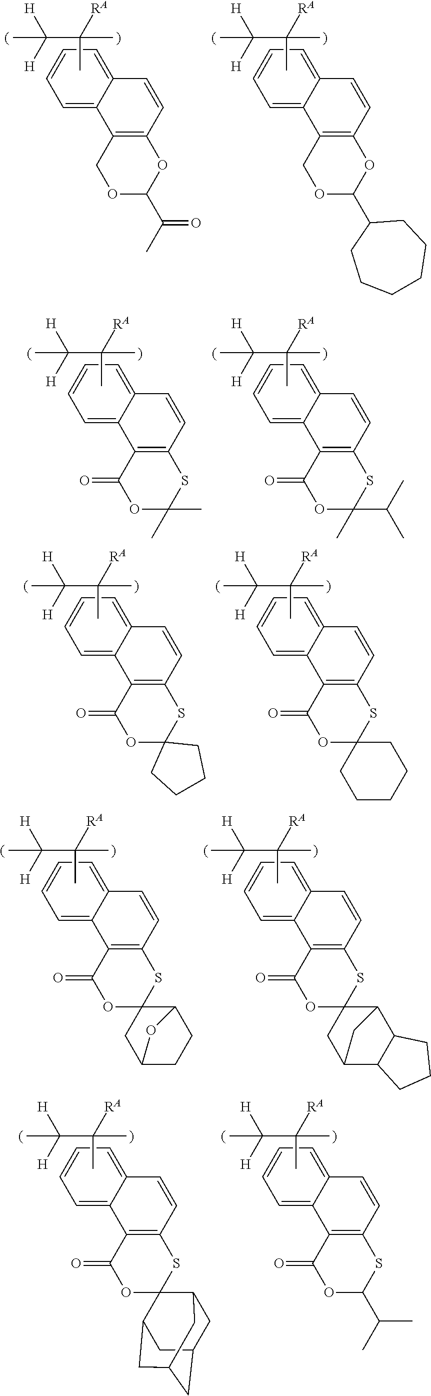

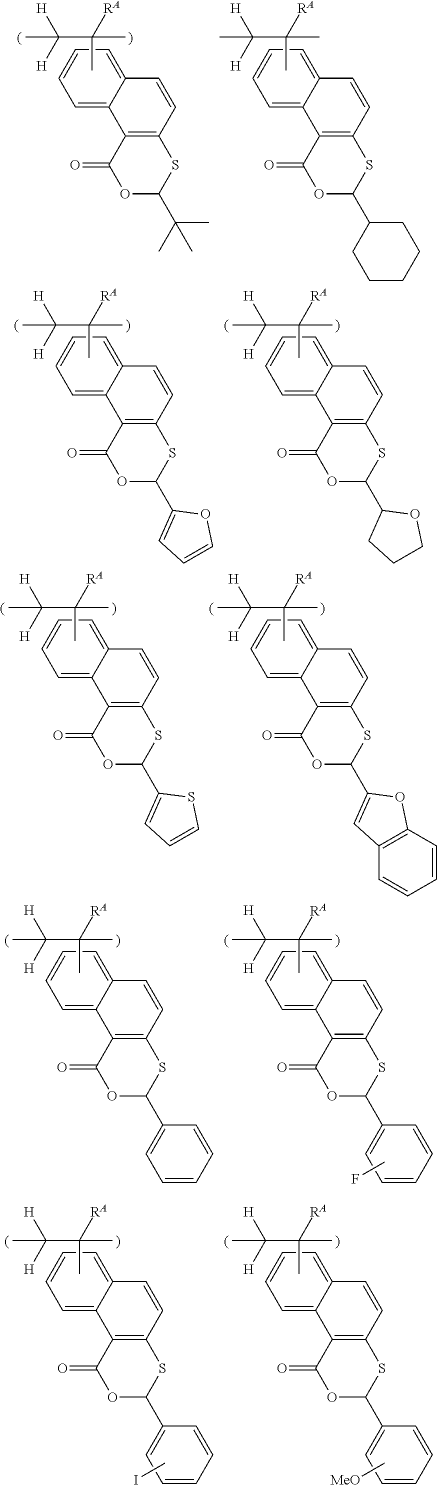

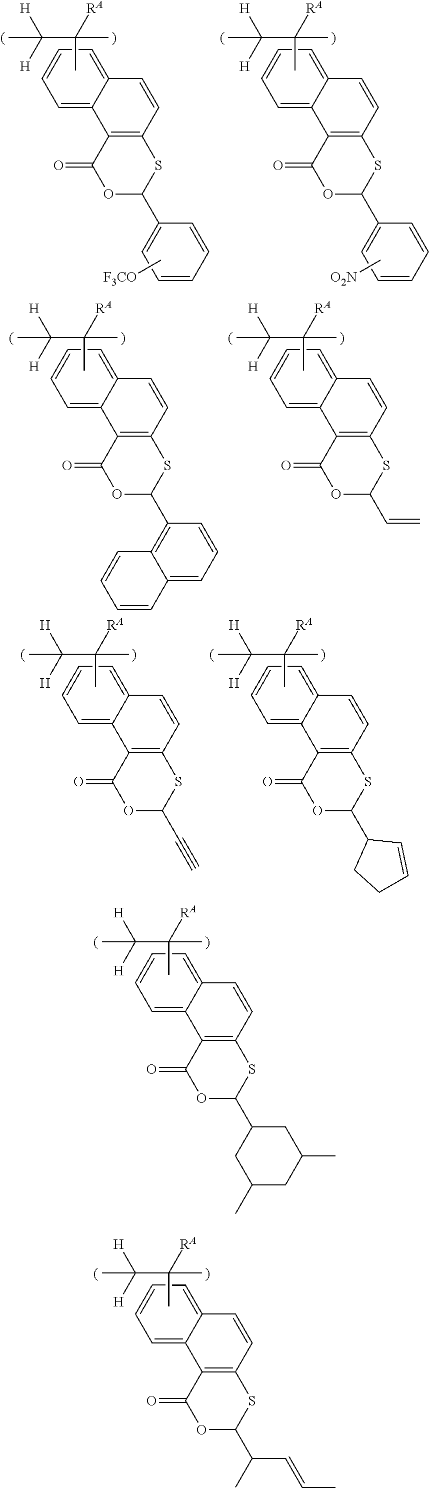

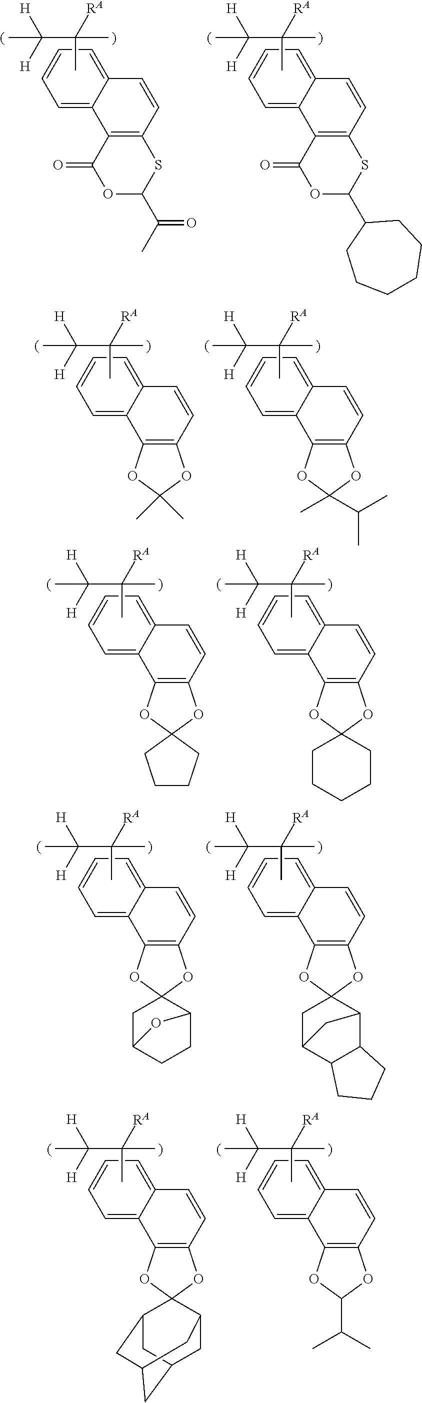

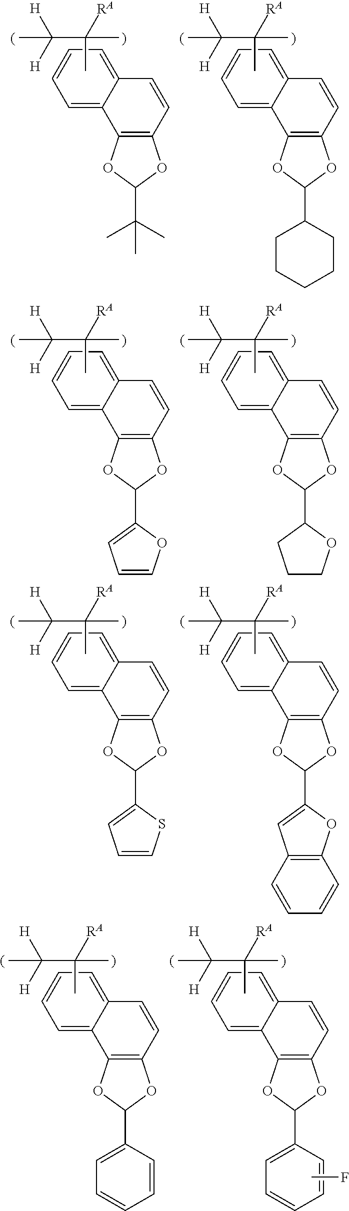

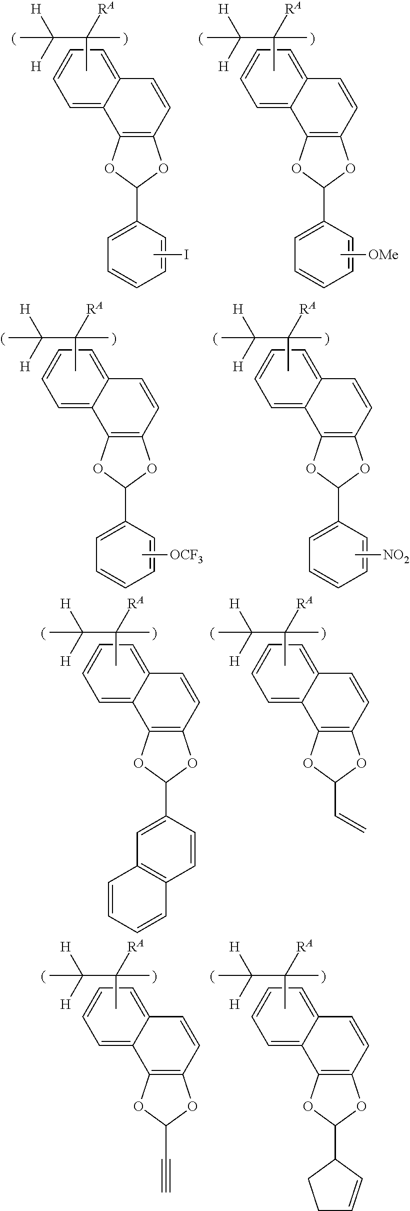

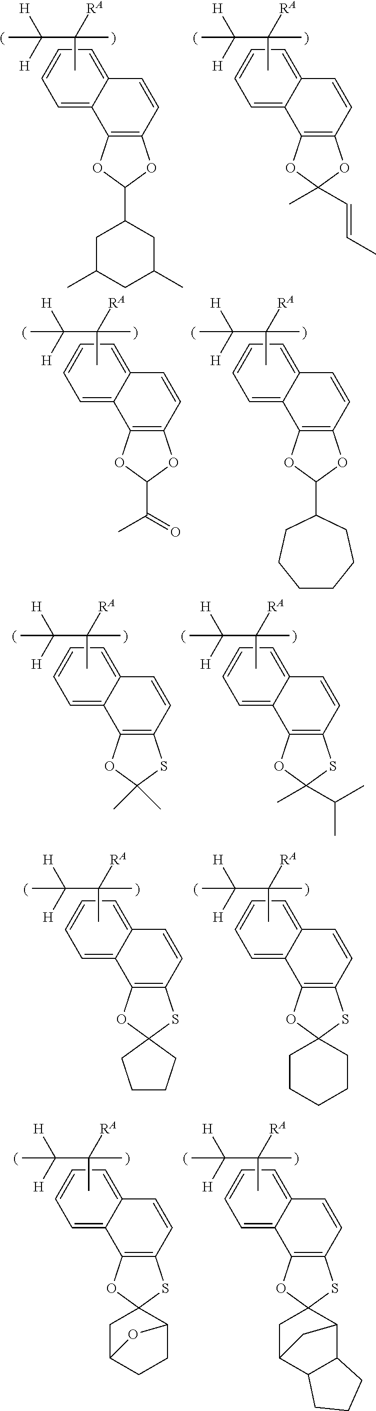

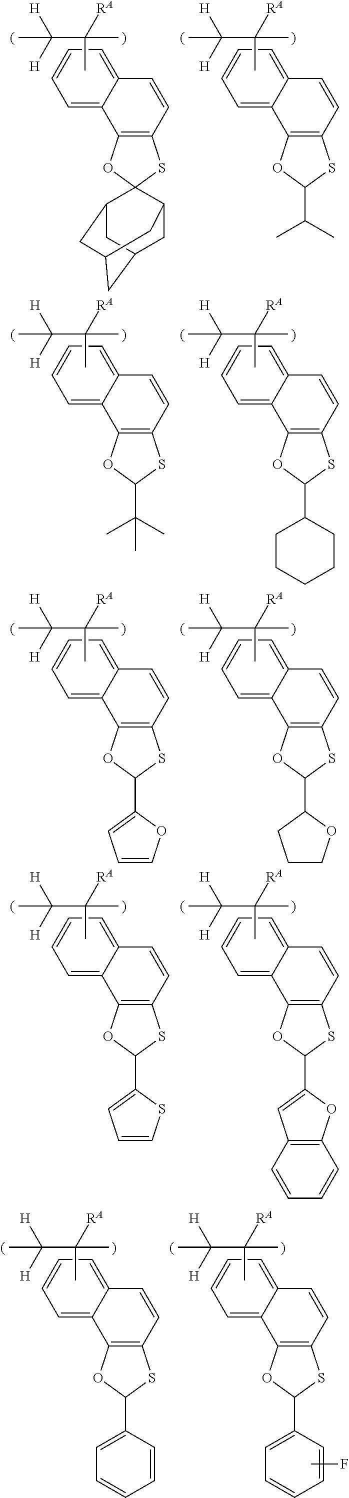

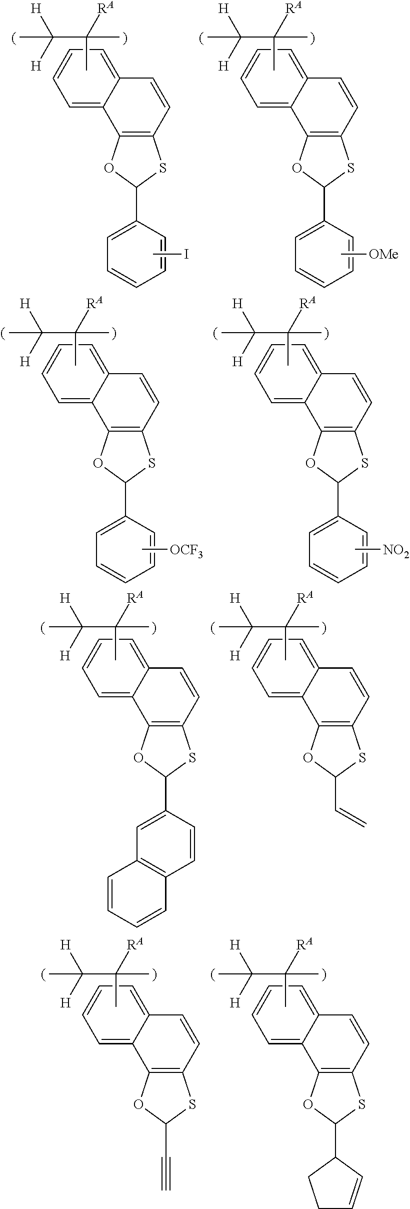

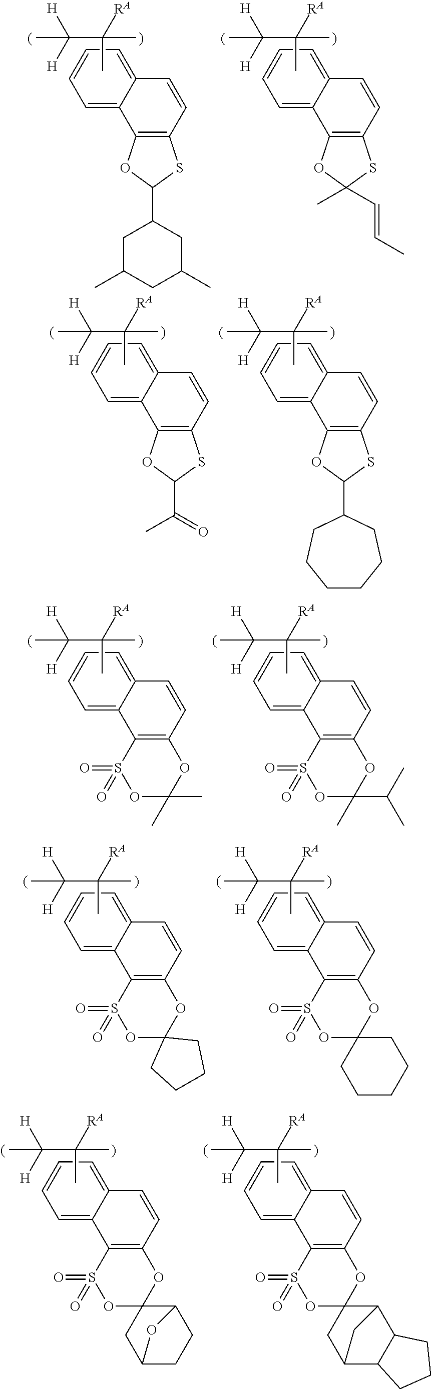

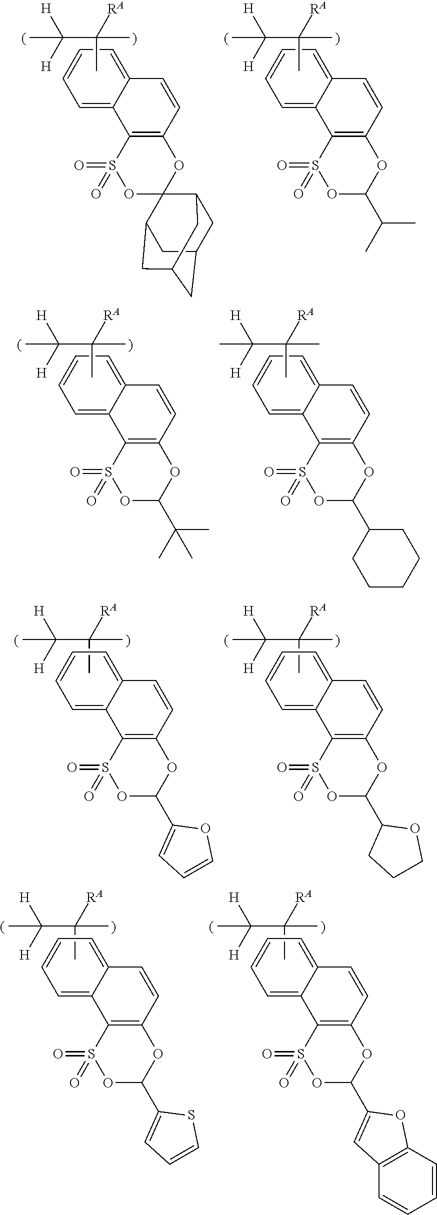

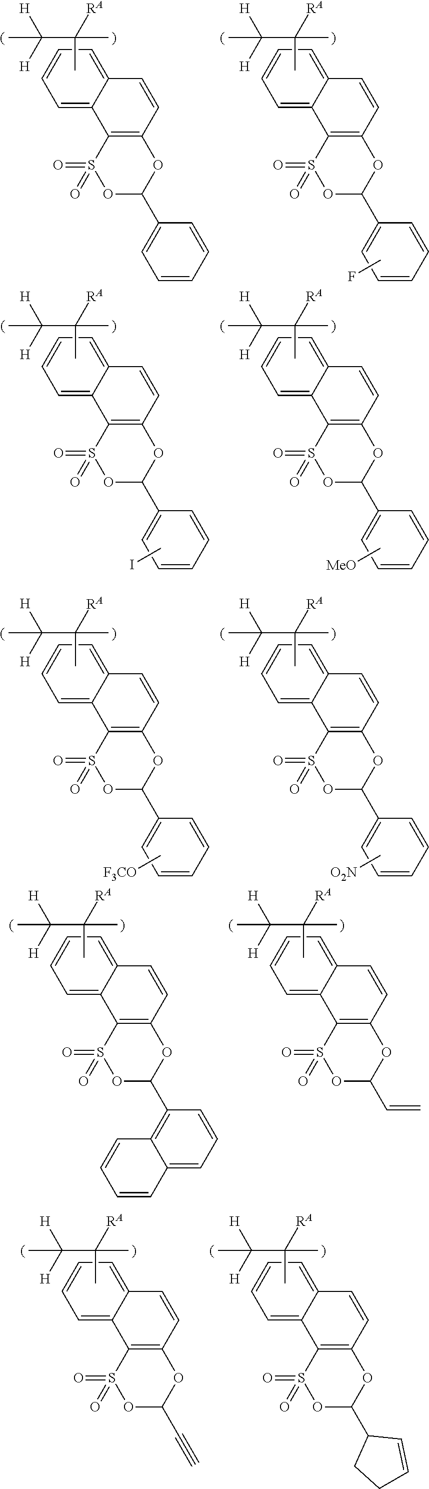

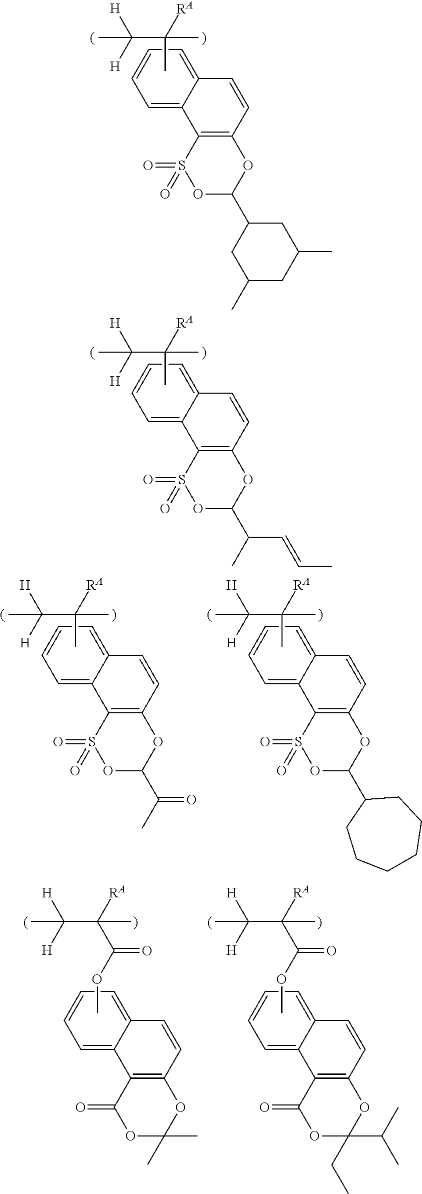

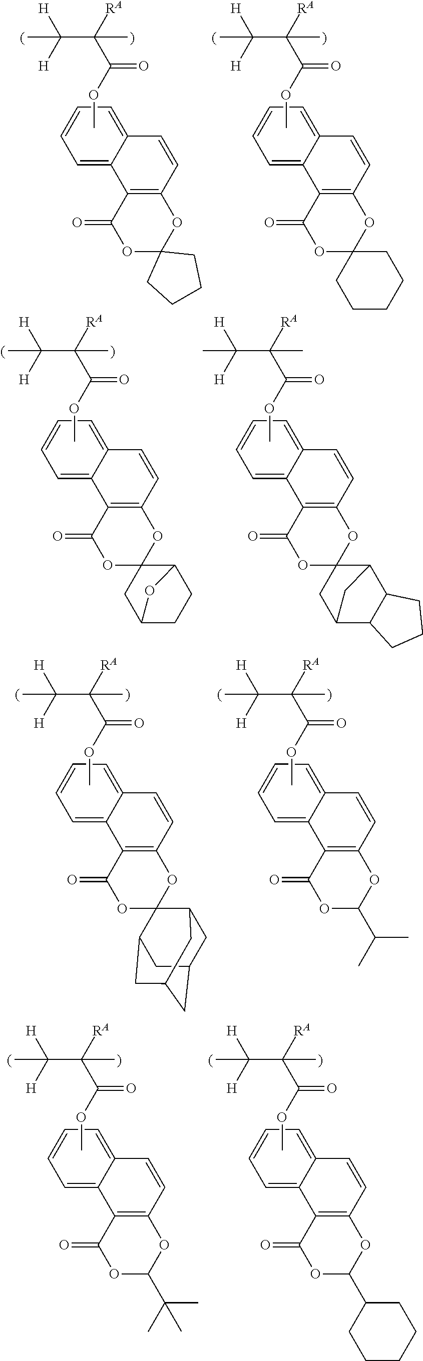

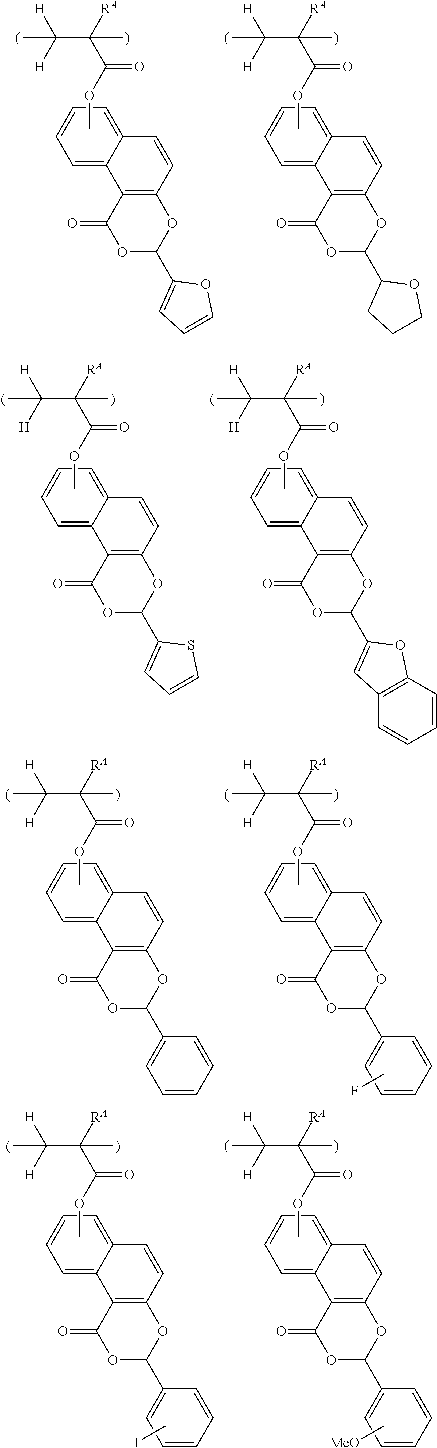

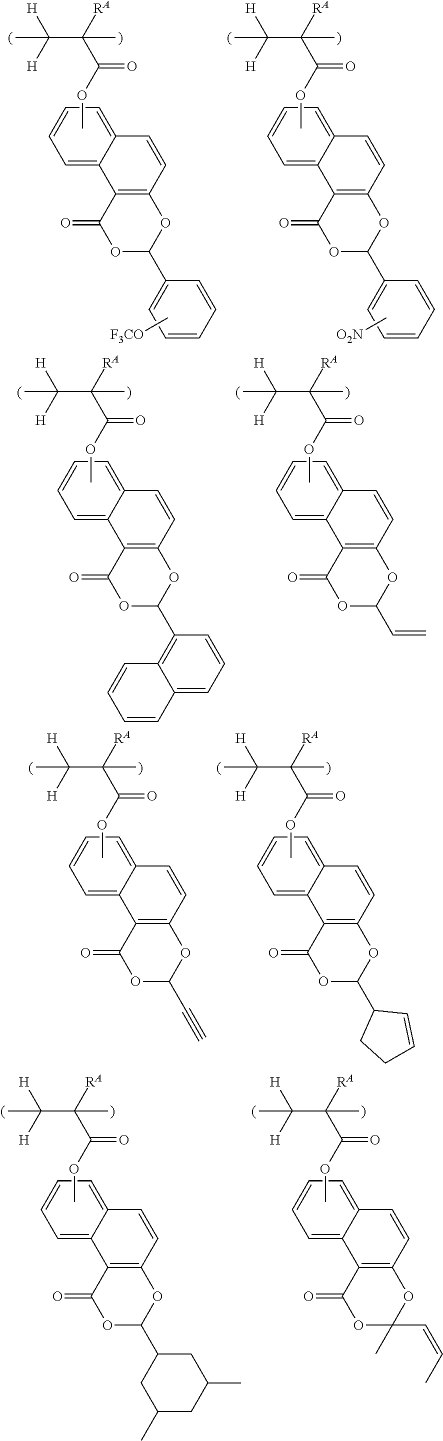

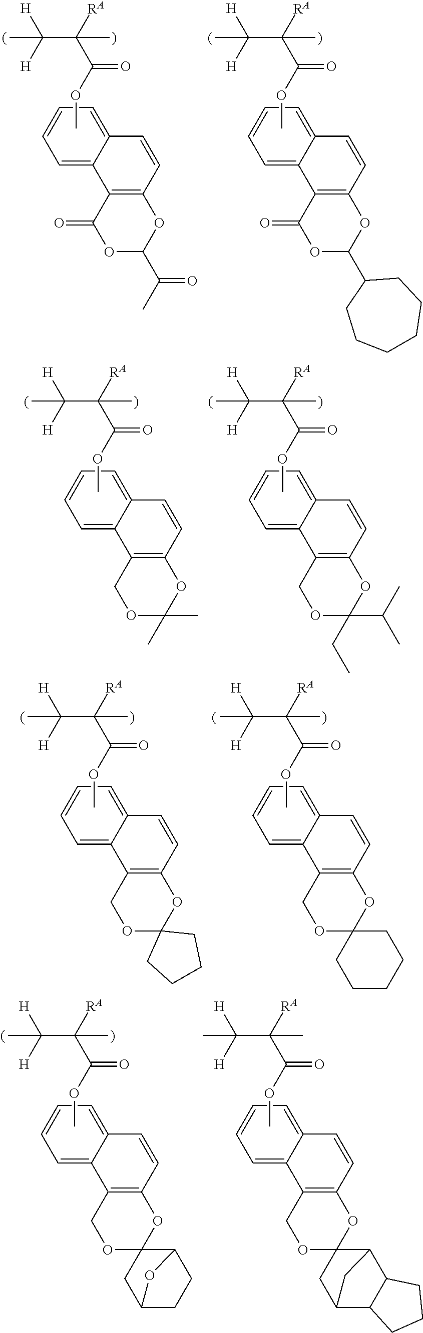

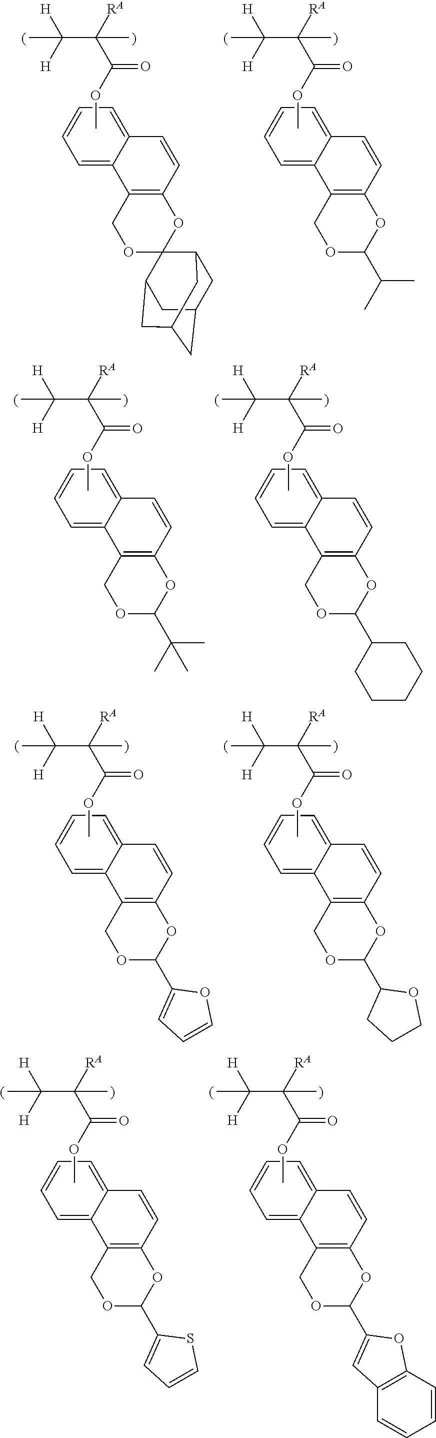

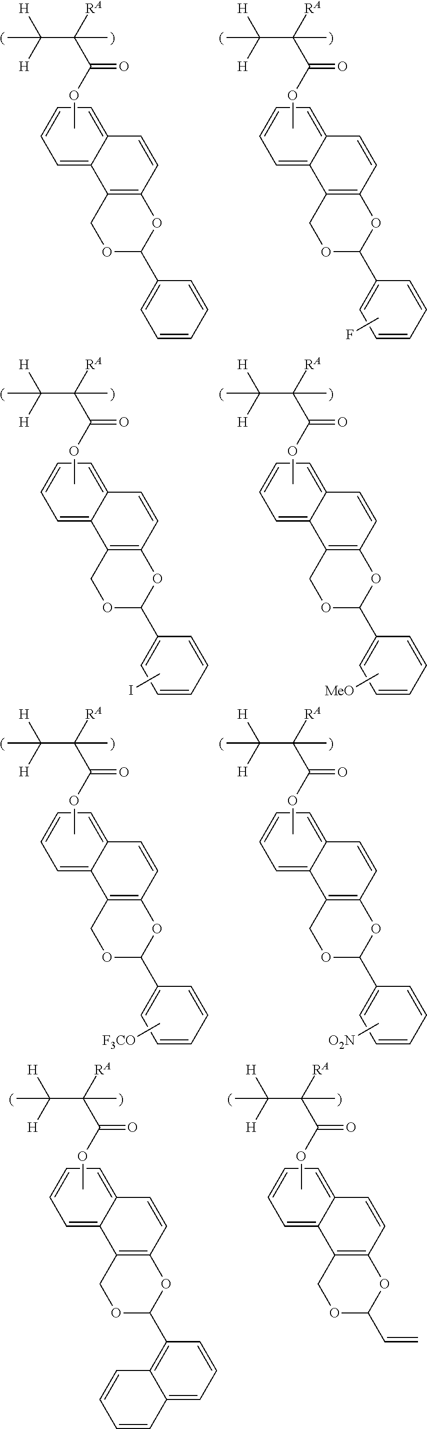

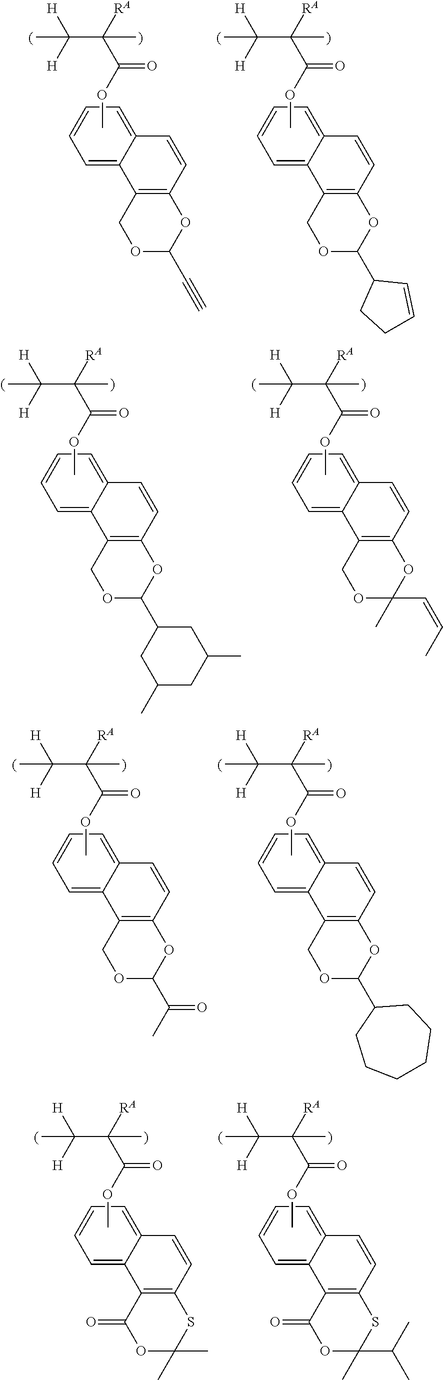

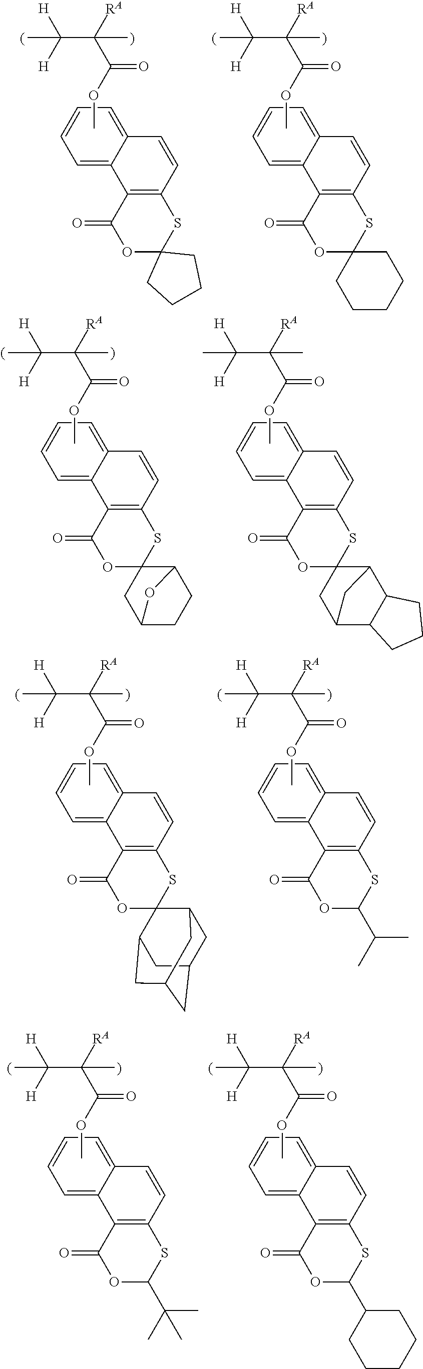

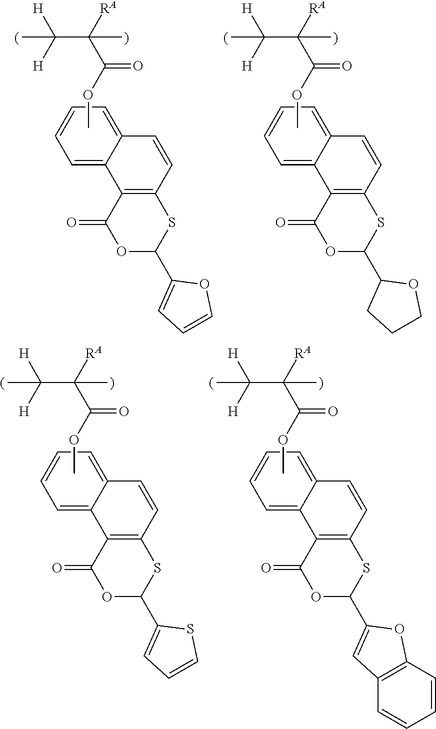

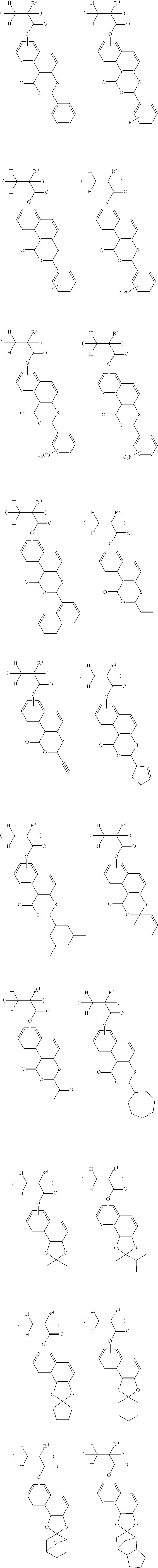

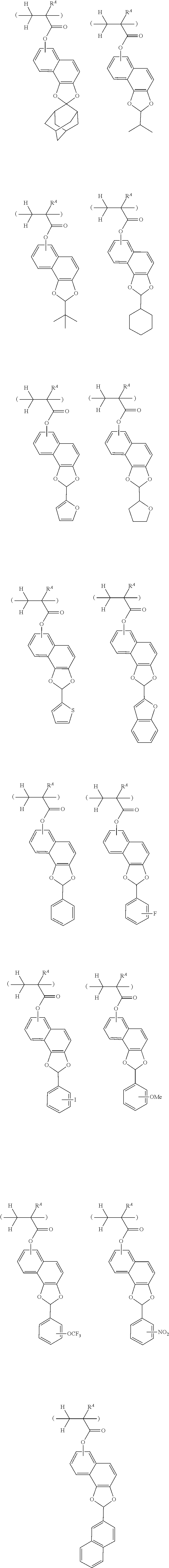

[chemical structure]

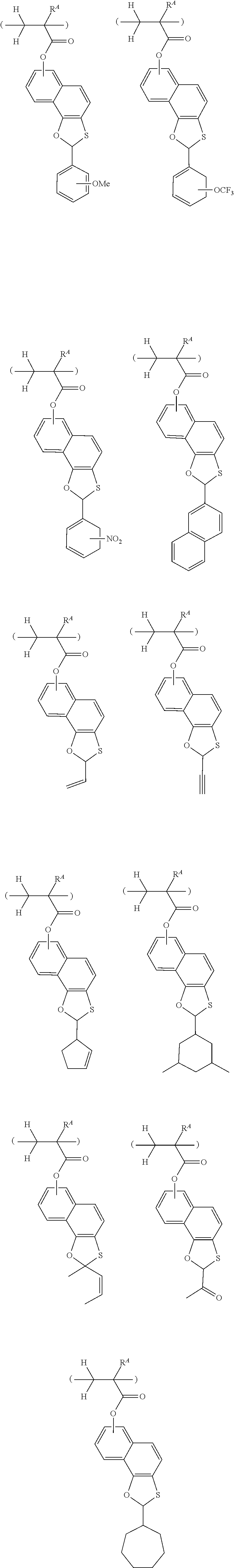

[chemical structure]

[chemical structure]

[chemical structure]

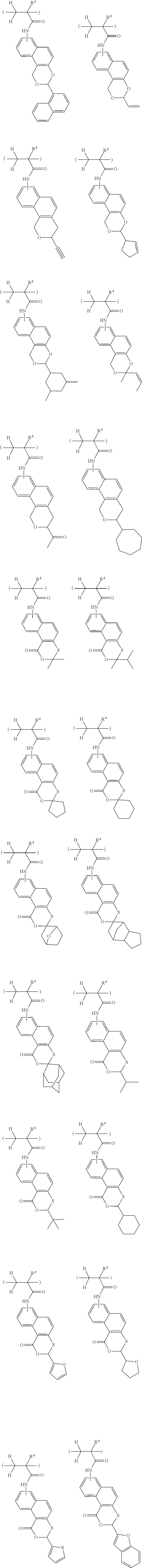

[chemical structure]

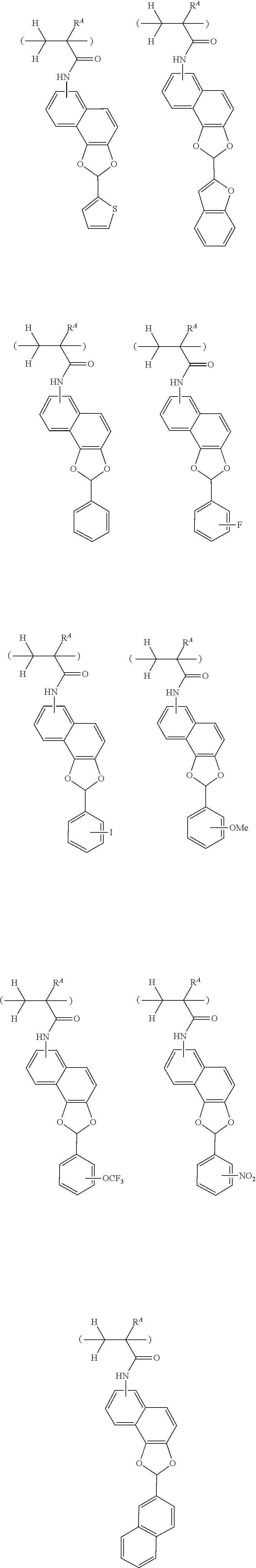

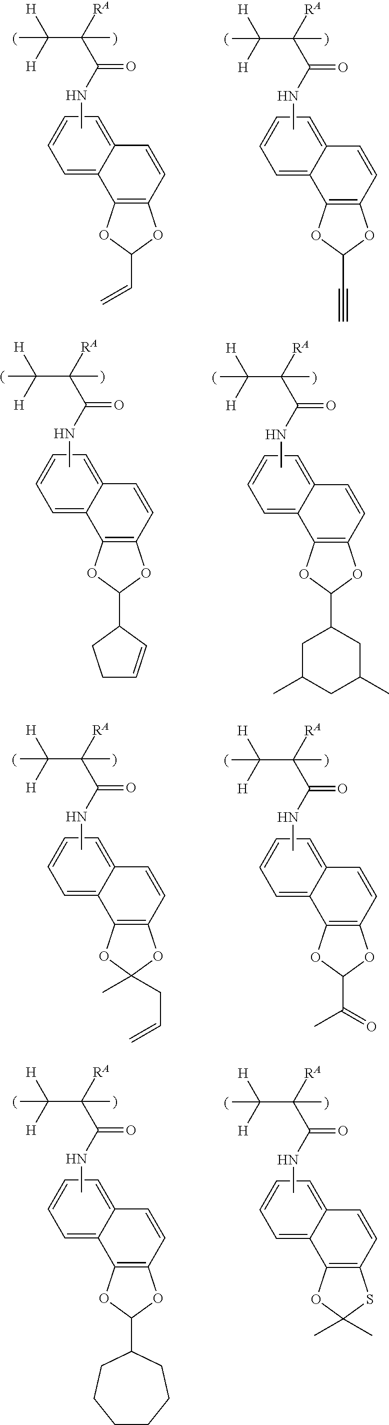

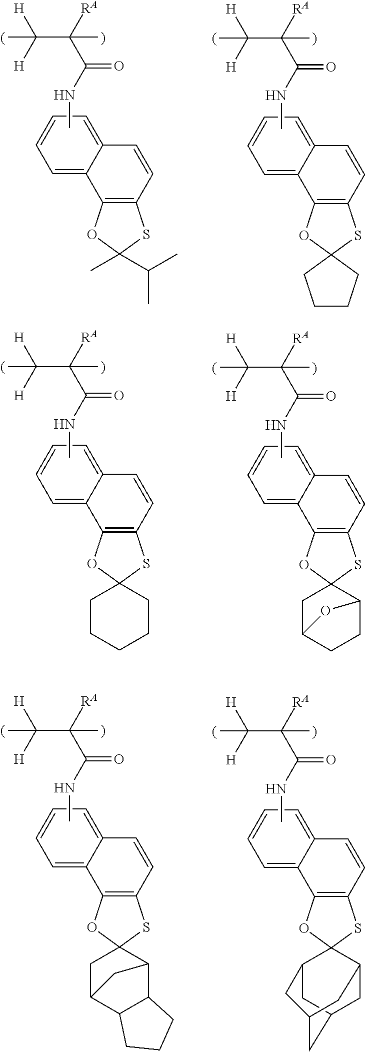

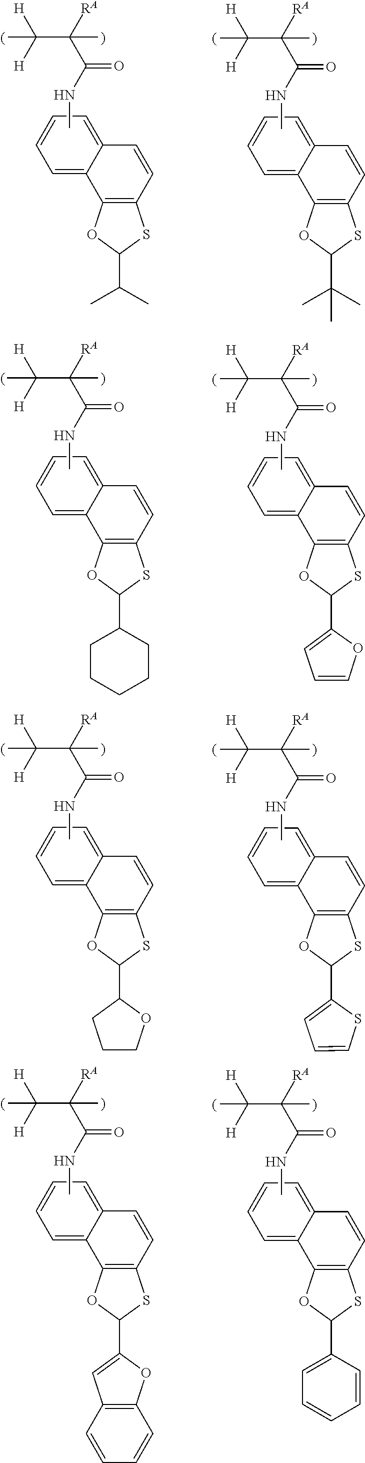

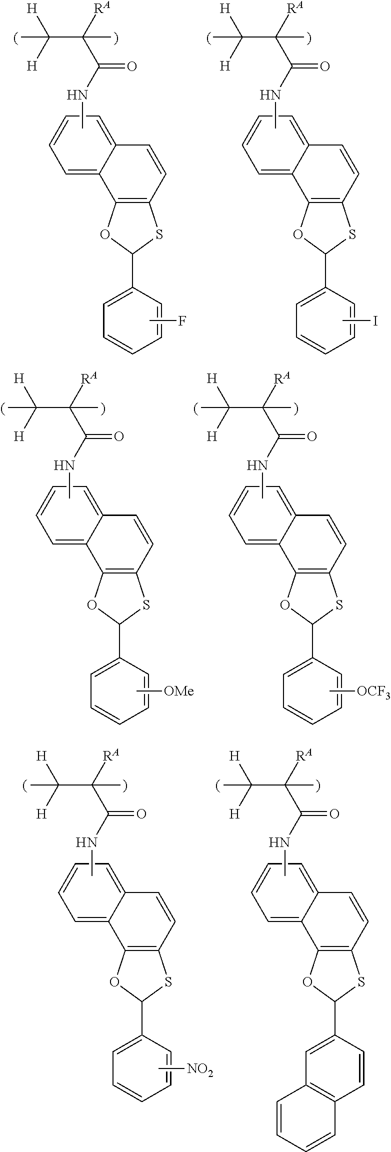

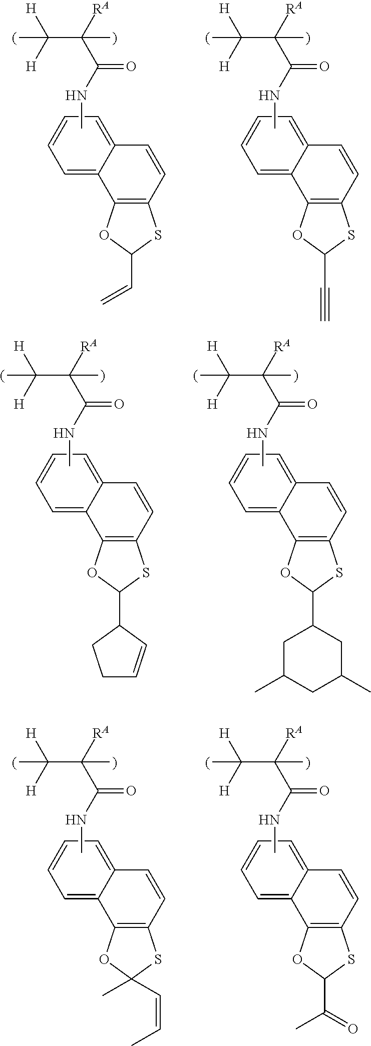

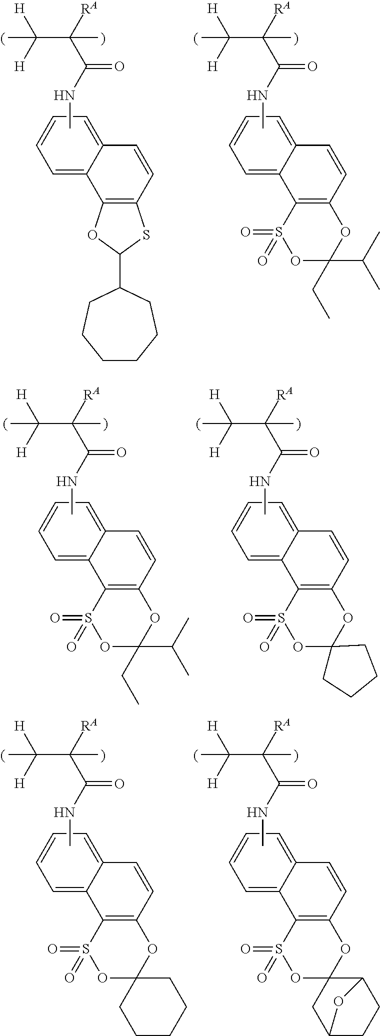

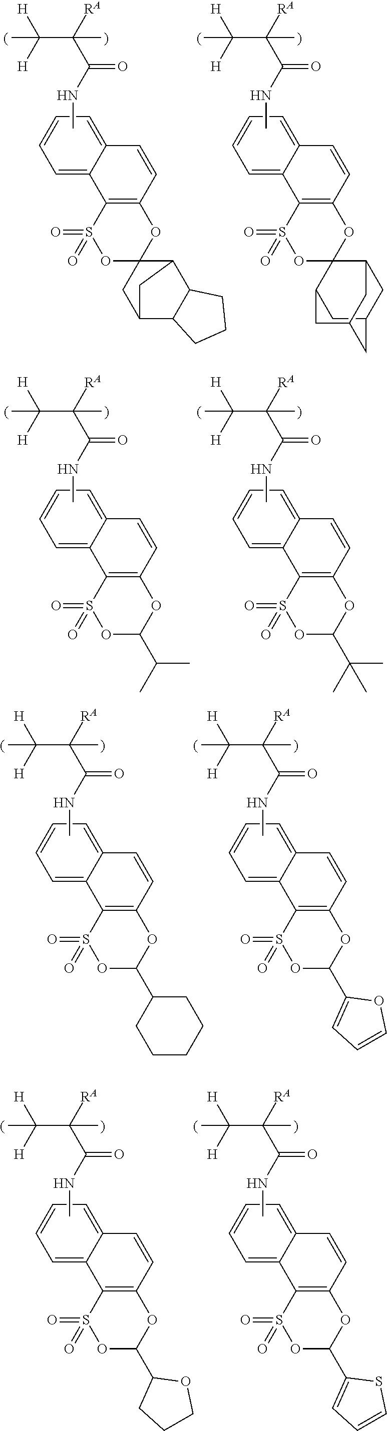

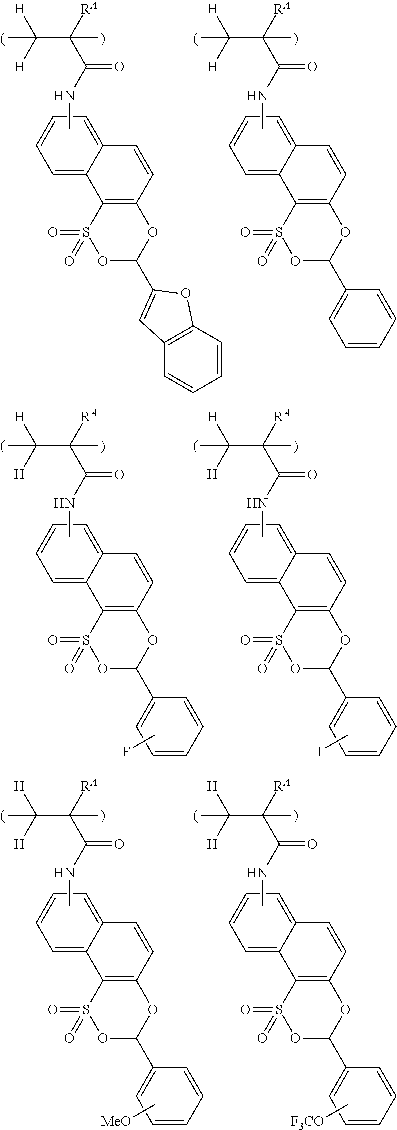

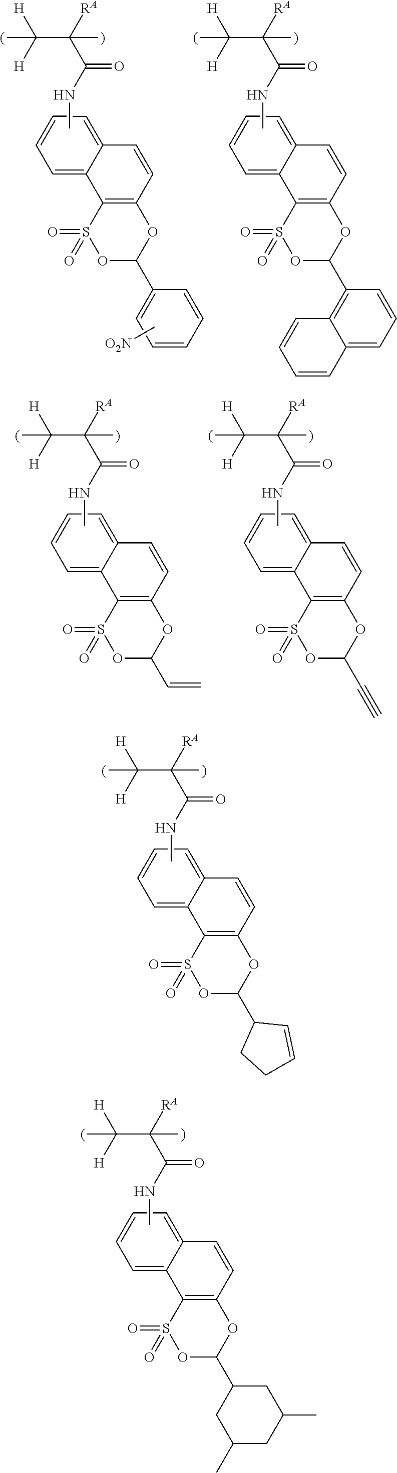

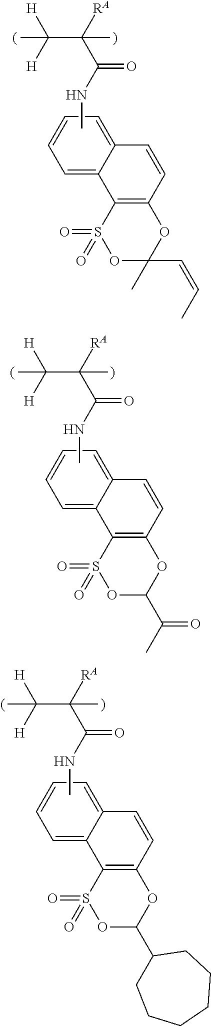

The polymer preferably further contains a repeat unit having the following formula (c) (hereinafter, also referred to as a repeat unit c).

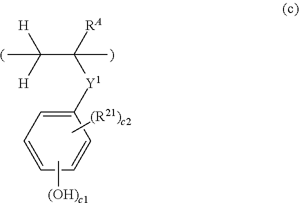

In the formula (c), R^{A} is a hydrogen atom, a fluorine atom, a methyl group, or a trifluoromethyl group. Y¹ is a single bond, *—C(═O)—O—, or *—C(═O)—N(H)—. * represents a bond with a carbon atom of a main chain. R²¹ is a halogen atom, a carboxy group, a nitro group, a cyano group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, c1 is 1, 2, 3, or 4. c2 is 0, 1, 2, or 3. Provided that 1≤c1+c2≤5.

Specific examples of the repeat unit c include, but are not limited to, those listed below. Note that, in the following formulas, R^{A} is as defined above.

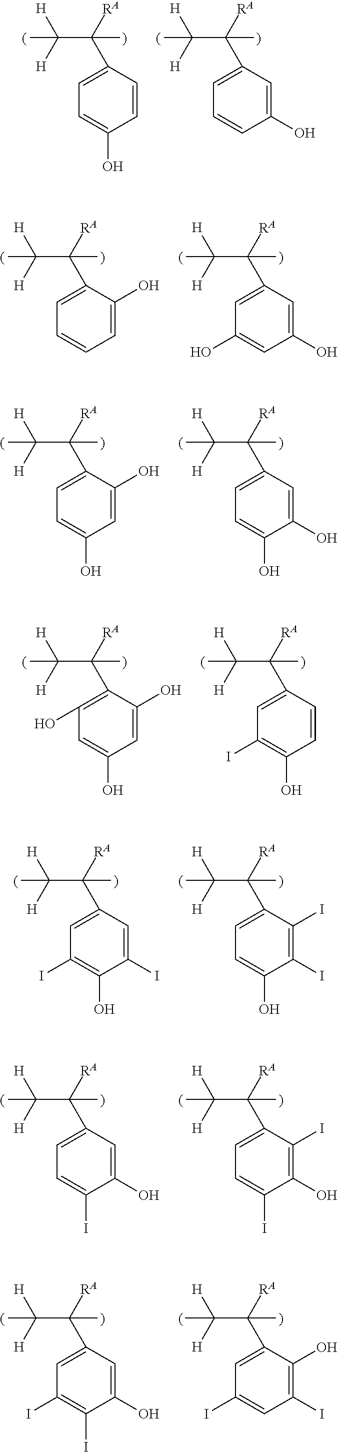

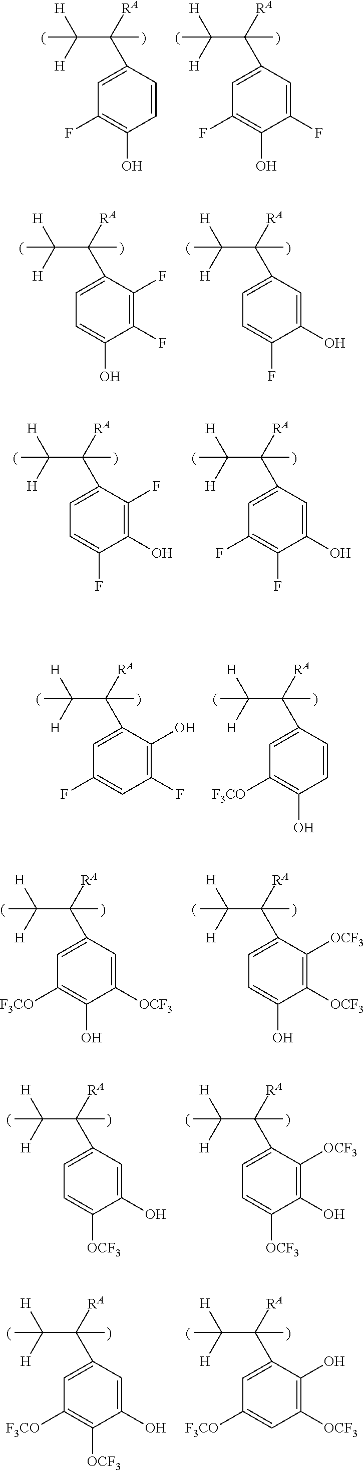

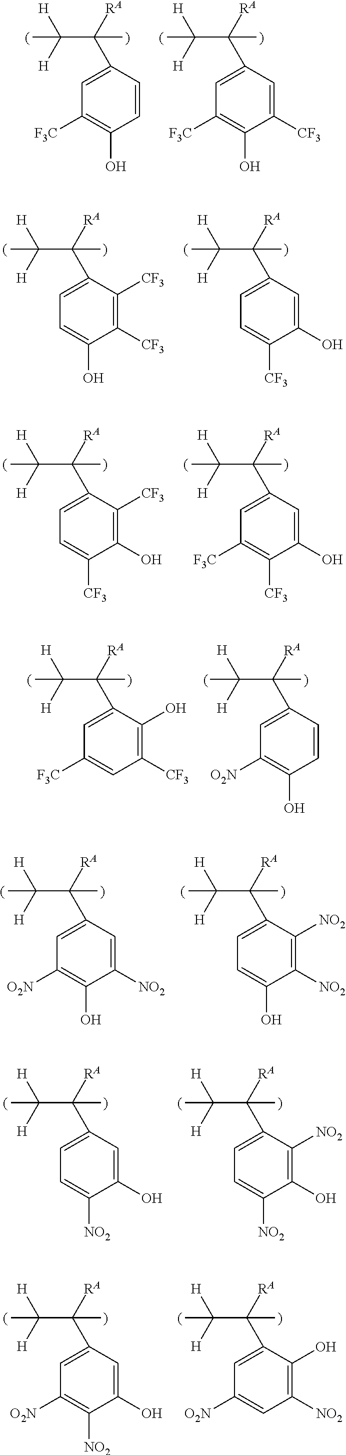

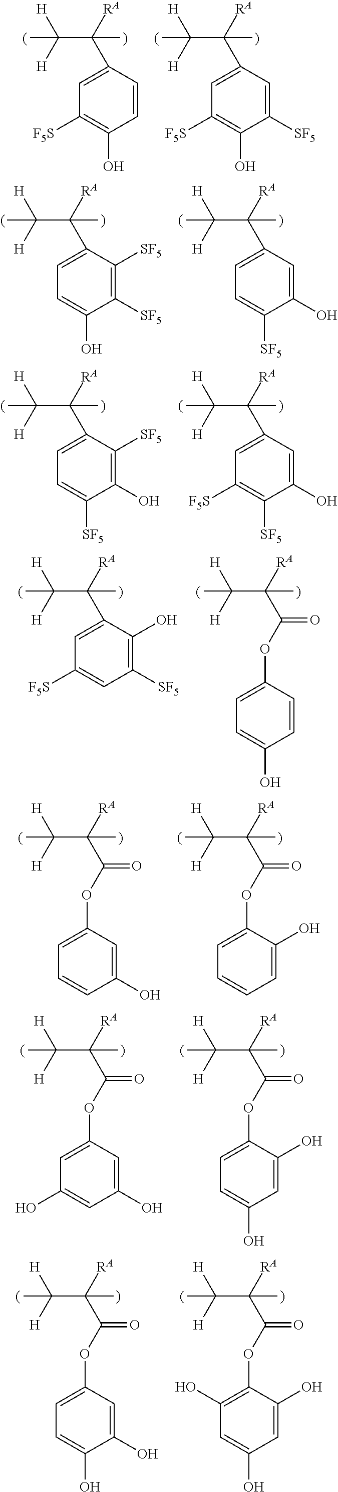

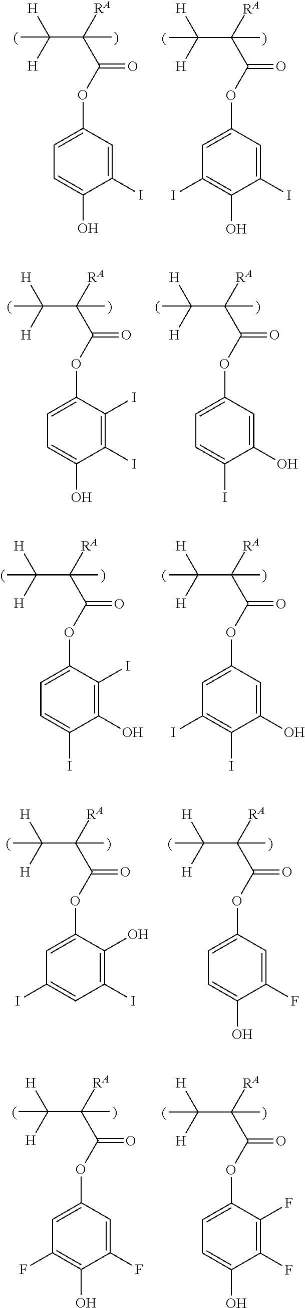

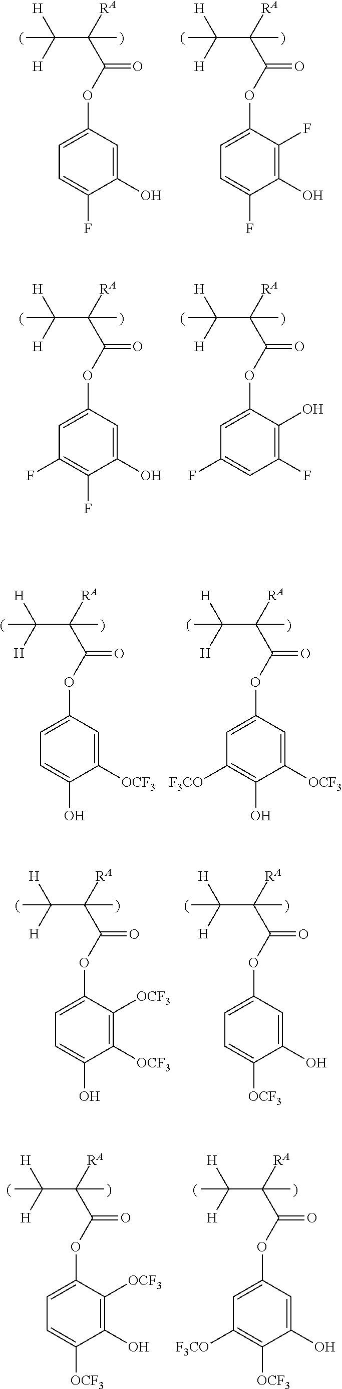

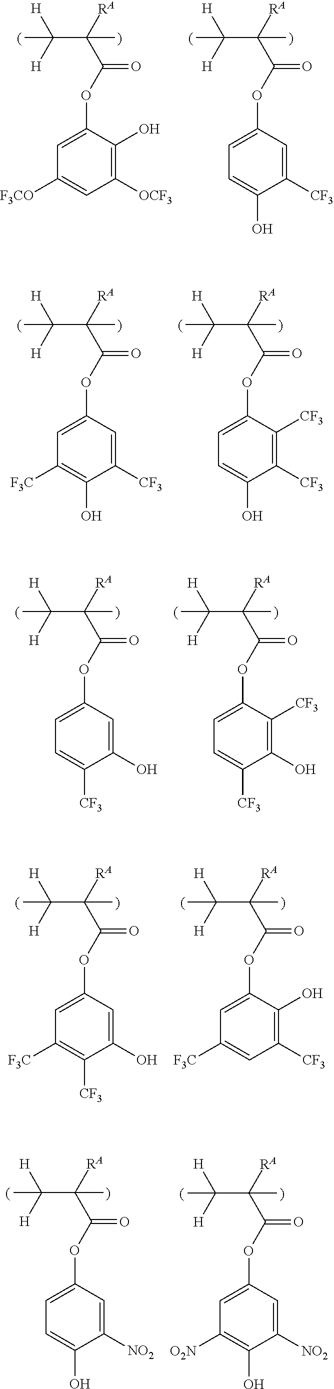

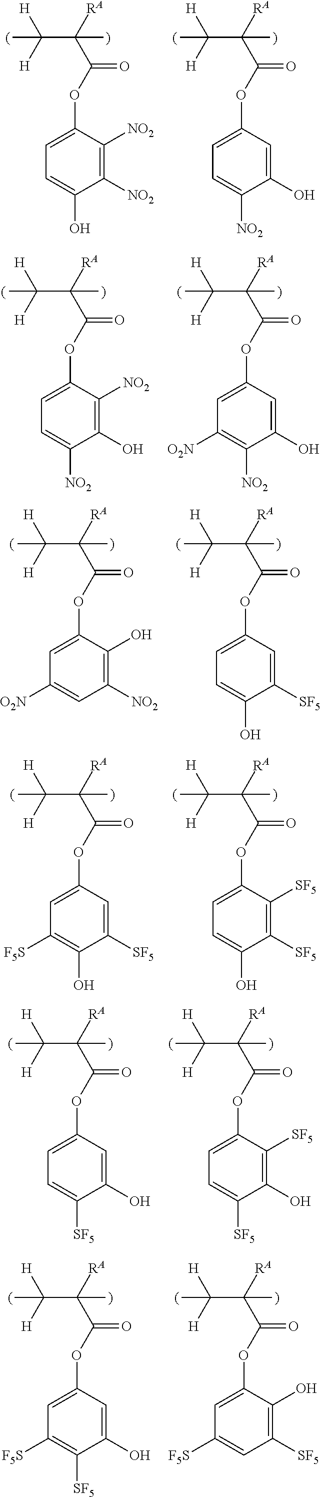

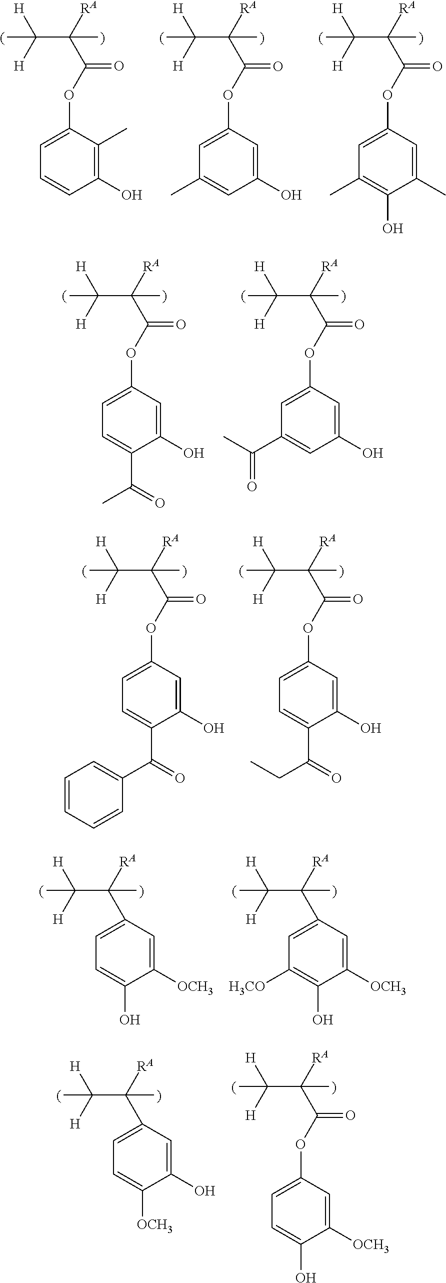

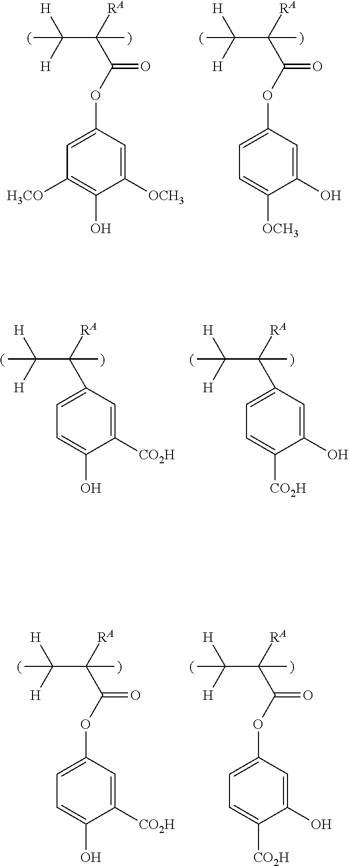

The polymer preferably further contains a repeat unit derived from an onium salt monomer containing a fluorosulfonic acid anion having a polymerizable group and at least one iodine atom and a sulfonium cation (hereinafter, also referred to as a repeat unit d).

Specific examples of the anion of the repeat unit d include structures described in paragraphs [0023] to [0029] of JP 6973274, paragraphs [0032] to [0038] of JP-A 2023-172928, and paragraphs [0032] to [0047] of JP-A 2024-043941.

Preferable specific examples of the anion of the repeat unit d include an anion having the following formula (d1).

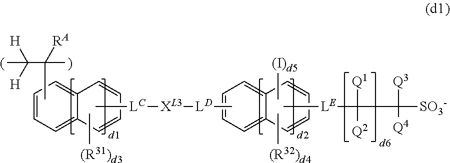

In the formula (d1), d1 is 0 or 1. When d1 is 0, it is a benzene ring, and when d1 is 1, it is a naphthalene ring, but a benzene ring in which d1 is 0 is preferable from a viewpoint of solvent solubility. d2 is 0 or 1. When d2 is 0, it is a benzene ring, and when d2 is 1, it is a naphthalene ring, but a benzene ring in which d2 is 0 is preferable from a viewpoint of solvent solubility. d3 is 0, 1, 2, 3, or 4. d3 is preferably 0, 1, or 2, and more preferably 0 or 1 from a viewpoint of raw material procurement. d4 is 0, 1, 2, 3, or 4, preferably 0, 1, 2, or 3, more preferably 0, 1, or 2, and still more preferably 0 or 1. d5 is 1, 2, 3, 4, 5, or 6. The larger the number of iodine atoms in the anion structure, the higher the absorption particularly for EUV, but since there is a concern that solvent solubility is poor and precipitation occurs in a resist composition, d5 is preferably 1, 2, or 3, and more preferably 1 or 2. Provided that 1≤d4+d5≤4 when d2 is 0, and 1≤d4+d5≤6 when d2 is 1. d6 is 0, 1, 2, 3, or 4, preferably 0, 1, 2, or 3, and more preferably 1.

In the formula (d1), R^{A} is a hydrogen atom, a fluorine atom, a methyl group, or a trifluoromethyl group. Among these, R^{A} is preferably a hydrogen atom or a methyl group, and more preferably a hydrogen atom.

In the formula (d1), an iodine atom in the aromatic ring of the anion is preferably bonded to an ortho position of a carbon atom to which L^{D} is bonded. Since the iodine atom is an element having a large atomic radius, rotation of a bonding axis between an aromatic ring to which the polymerizable group is bonded and an aromatic ring to which the iodine atom is bonded is suppressed, and rigidity of the polymer is improved.

In the formula (d1), R³¹ is a halogen atom or a C₁-C₂₀ hydrocarbyl group which may contain a heteroatom. The halogen atom is preferably a fluorine atom, a chlorine atom, a bromine atom, or an iodine atom, and more preferably a fluorine atom or an iodine atom. The hydrocarbyl group may be saturated or unsaturated, and may be linear, branched, or cyclic. Specific examples thereof include: a C₁-C₂₀ alkyl group such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, or an icosyl group; a C₃-C₂₀ cyclic saturated hydrocarbyl group such as a cyclopropyl group, a cyclopentyl group, a cyclohexyl group, a cyclopropylmethyl group, a 4-methylcyclohexyl group, a cyclohexylmethyl group, a norbornyl group, or an adamantyl group; a C₂-C₂₀ alkenyl group such as a vinyl group, a 1-propenyl group, a 2-propenyl group, a butenyl group, or a hexenyl group; a C₃-C₂₀ cyclic unsaturated hydrocarbyl group such as a cyclohexenyl group; a C₆-C₂₀ aryl group such as a phenyl group or a naphthyl group; a C₇-C₂₀ aralkyl group such as a benzyl group, a 1-phenylethyl group, or a 2-phenylethyl group; and a group obtained by combining these groups. Some or all of hydrogen atoms of the hydrocarbyl group may be replaced with a group containing a heteroatom such as an oxygen atom, a sulfur atom, a nitrogen atom, or a halogen atom, some of (—CH₂—)s in the hydrocarbyl group may be replaced with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like. When d3 is 2, 3, or 4, R³¹s may be the same as or different from each other.

When d3 is 2, 3, or 4, a plurality of R³¹s may be bonded to each other to form a ring together with aromatic ring carbon atoms to which they are bonded. Specific examples of the ring formed at this time include a cyclopropane ring, a cyclobutane ring, a cyclopentane ring, a cyclohexane ring, a norbornane ring, and an adamantane ring. Some or all of hydrogen atoms of the ring may be replaced with a group containing a heteroatom such as an oxygen atom, a sulfur atom, a nitrogen atom, or a halogen atom, some of (—CH₂—)s in the ring may be replaced with a group containing a heteroatom such as an oxygen atom, a sulfur atom, or a nitrogen atom, and as a result, the ring may contain a hydroxy group, a fluorine atom, a chlorine atom, a bromine atom, an iodine atom, a cyano group, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like.

In the formula (d1), R³² is a halogen atom other than an iodine atom, or a C₁-C₂₀ hydrocarbyl group which may contain a heteroatom. Specific examples of the halogen atom other than an iodine atom include a fluorine atom, a chlorine atom, a bromine atom. The hydrocarbyl group may be saturated or unsaturated, and may be linear, branched, or cyclic. Specific examples thereof include, but are not limited to, those similar to those exemplified as the hydrocarbyl group of R³¹. When d4 is 2, 3, or 4, R³²s may be the same as or different from each other.

When d4 is 2, 3, or 4, a plurality of R³²s may be bonded to each other to form a ring together with carbon atoms to which they are bonded. The ring is preferably a 5- to 8-membered ring.

In the formula (d1), L^{C}, L^{D}, and L^{E} are each independently a single bond, an ether bond, an ester bond, a sulfonate bond, a sulfonamide bond, a carbonate bond, or a carbamate bond. Among these, L^{C} is preferably a single bond, an ether bond, an ester bond, or a sulfonate bond, and more preferably an ester bond or a sulfonate bond. L^{D} is preferably a single bond, an ether bond, or an ester bond, and more preferably a single bond. L^{E} is preferably a single bond, an ether bond, an ester bond, or a sulfonate bond, and more preferably an ether bond or an ester bond.

In the formula (d1), X^{L3} is a single bond or a C₁-C₄₀ hydrocarbylene group which may contain a heteroatom. The hydrocarbylene group may be linear, branched, or cyclic, and specific examples thereof include an alkanediyl group, a cyclic saturated hydrocarbylene group, and an arylene group. Specific examples of the heteroatom include an oxygen atom, a nitrogen atom, and a sulfur atom. Specific examples of the C₁-C₄₀ hydrocarbylene group of X^{L3} which may contain a heteroatom include X^{L}-0 to X^{L}-61 exemplified as specific examples of the C₁-C₄₀ hydrocarbylene group of X^{L1} and X^{L2} which may contain a heteroatom in the description of the formula (Z). Among these, X^{L}-0 to X^{L}-22, X^{L}-29 to X^{L}-34, and X^{L}-47 to X^{L}-61 are preferable.

X^{L3} is preferably a single bond from a viewpoint of rigidity of a polymer to be obtained.

In the formula (d1), Q¹ and Q² are each independently a hydrogen atom, a fluorine atom, or a C₁-C₆ fluorinated saturated hydrocarbyl group. The C₁-C₆ fluorinated saturated hydrocarbyl group is preferably a trifluoromethyl group.

In the formula (d1), Q³ and Q⁴ are each independently a fluorine atom or a C₁-C₆ fluorinated saturated hydrocarbyl group. The C₁-C₆ fluorinated saturated hydrocarbyl group is preferably a trifluoromethyl group. Q³ and Q⁴ are each more preferably a fluorine atom.

Specific preferable examples of a partial structure of—[C(Q¹) (Q²)]_{d6}—C(Q³) (Q⁴)—SO₃⁻ in the formula (d1) include, but are not limited to, those listed below. Note that, in the following formulas, * represents a bond with L^{E}.

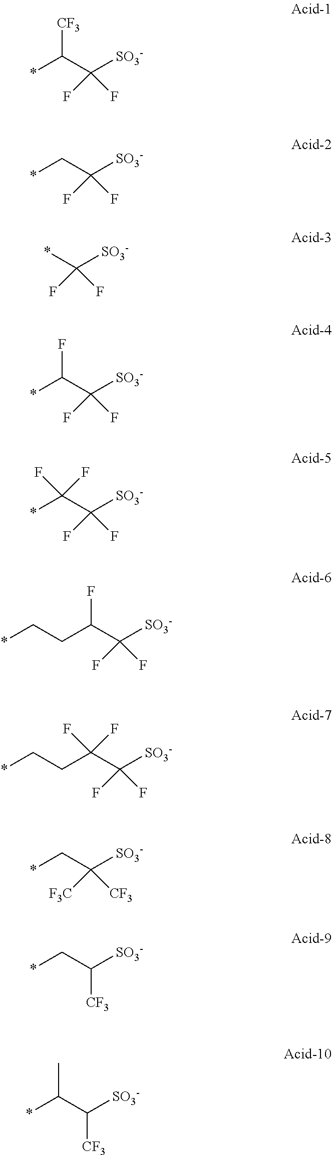

Among these, Acid-1 to Acid-7 are preferable, and Acid-1 to Acid-3, Acid-6, and Acid-7 are more preferable.

The anion having the formula (d1) preferably has the following formula (d1-1):

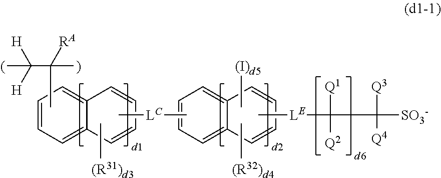

wherein d1 to d6, R^{A}, R³¹, R³², L^{C}, L^{E}, Q¹ to Q⁴, and Z⁺ are as defined above.

The anion having the formula (d1-1) preferably has the following formula (d1-2):

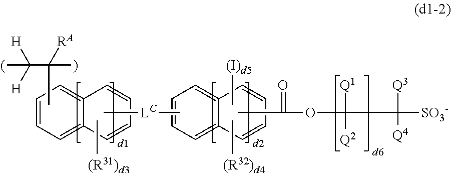

wherein d1 to d6, R^{A}, R³¹, R³², L^{C}, Q¹, Q², and Z⁺ are as defined above.

Specific examples of the anion having the formula (d1) include, but are not limited to, those listed below. Note that, in the following formulas, R^{A} and Q¹ are as defined above, and Me is a methyl group. Bonding positions of various substituents on an aromatic ring may be replaced with each other.

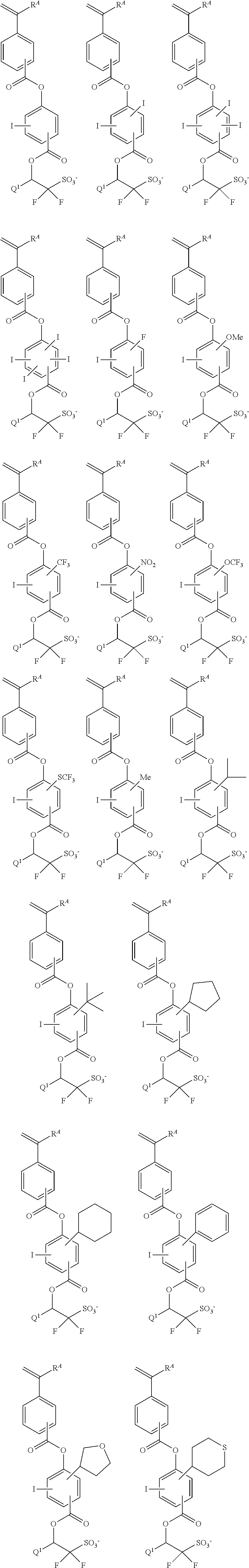

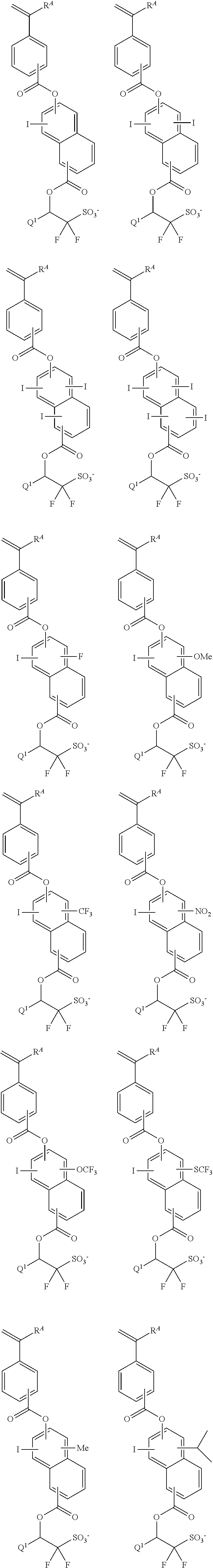

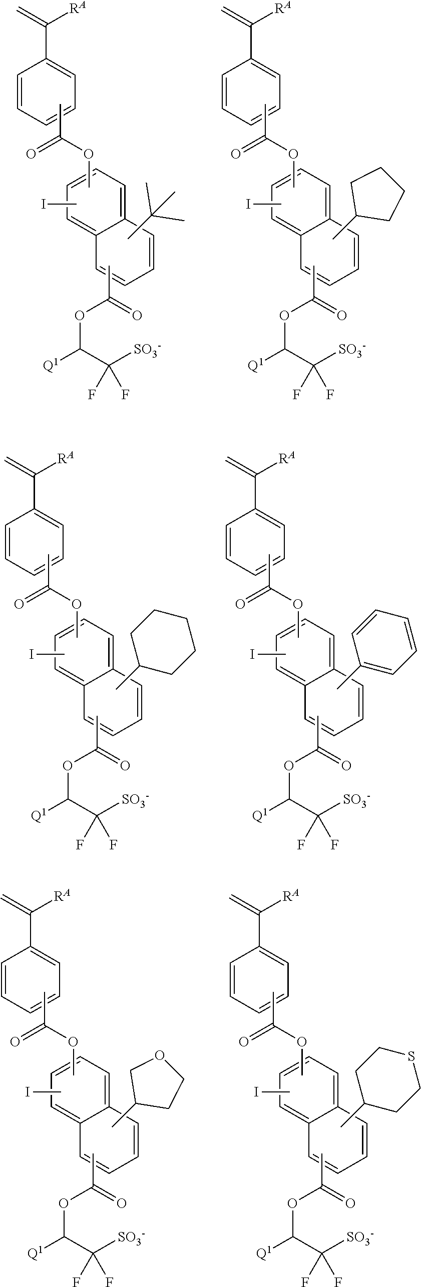

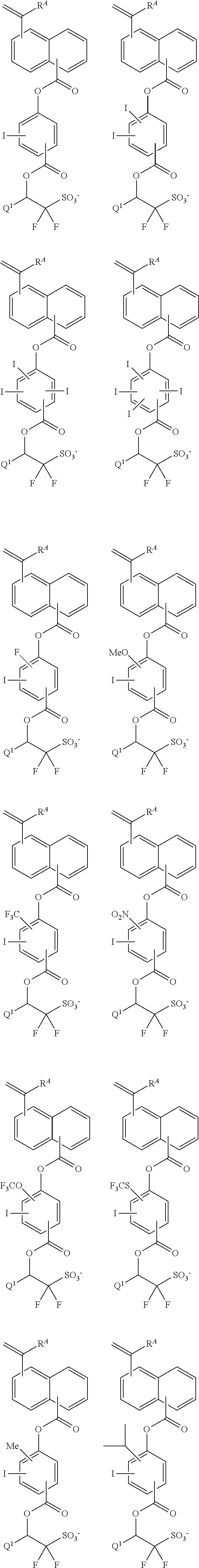

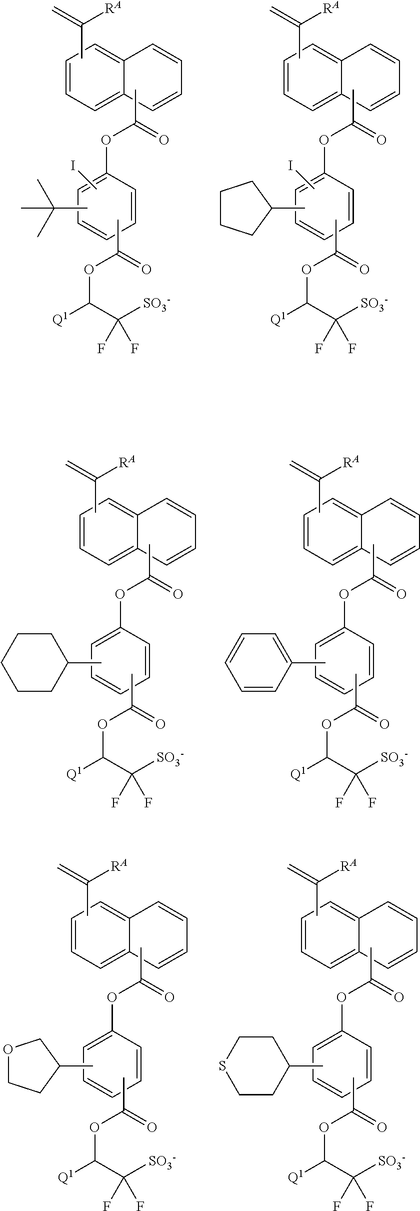

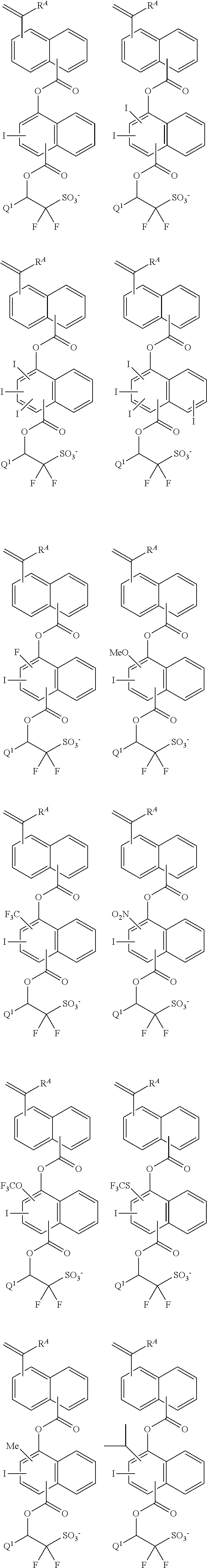

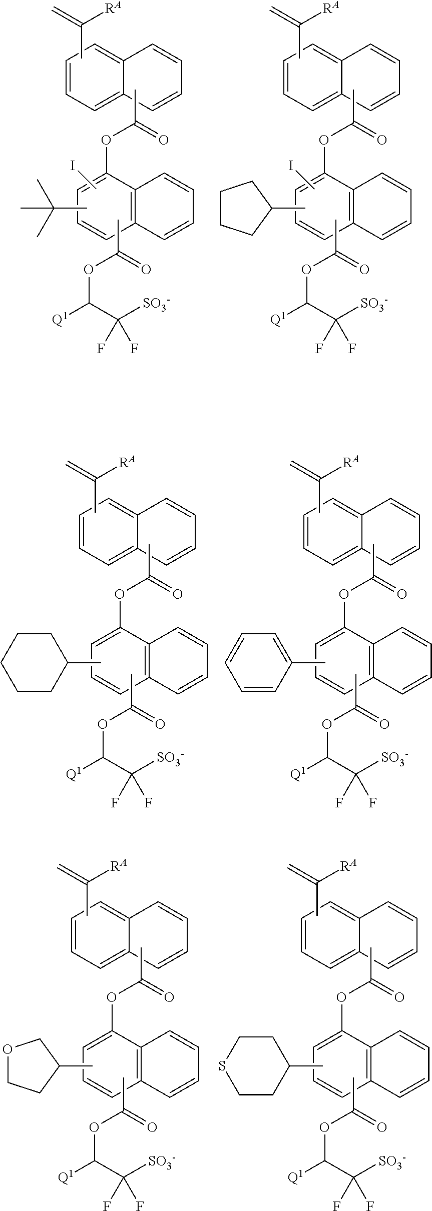

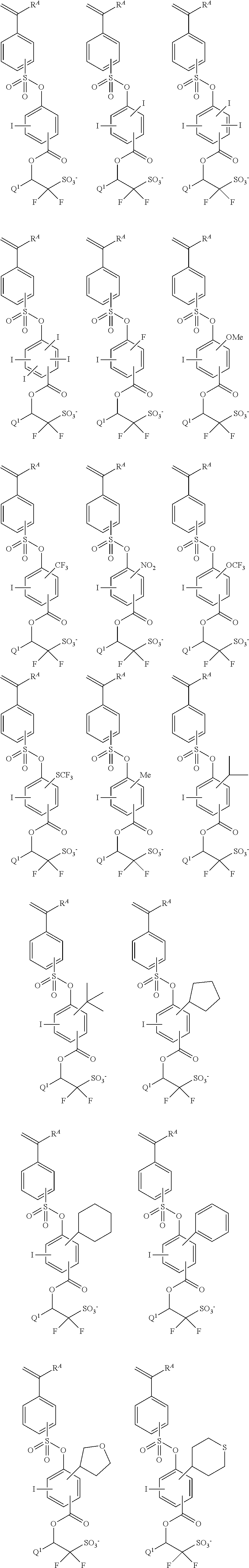

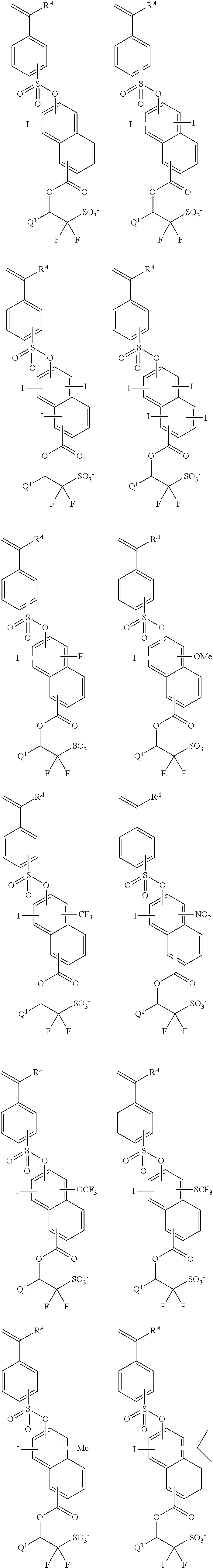

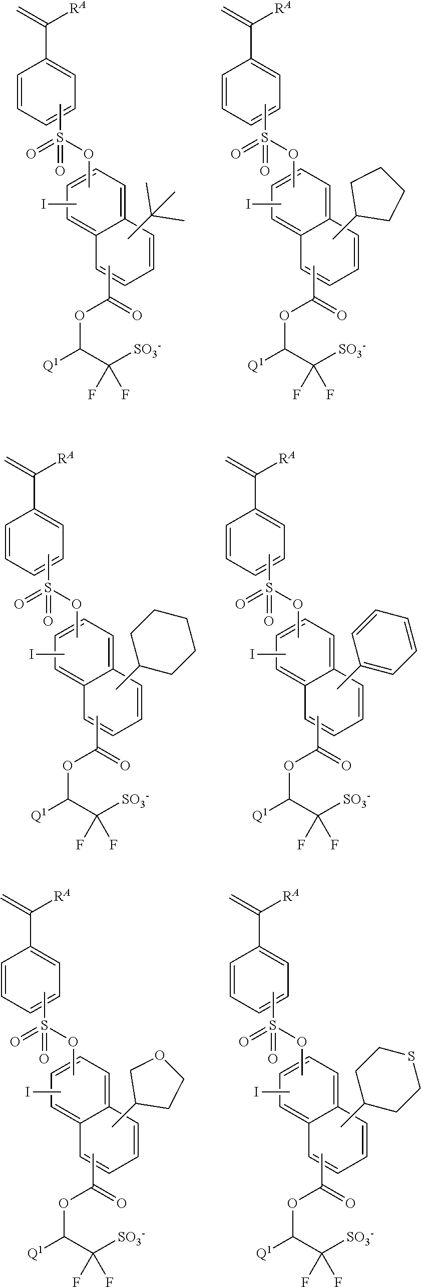

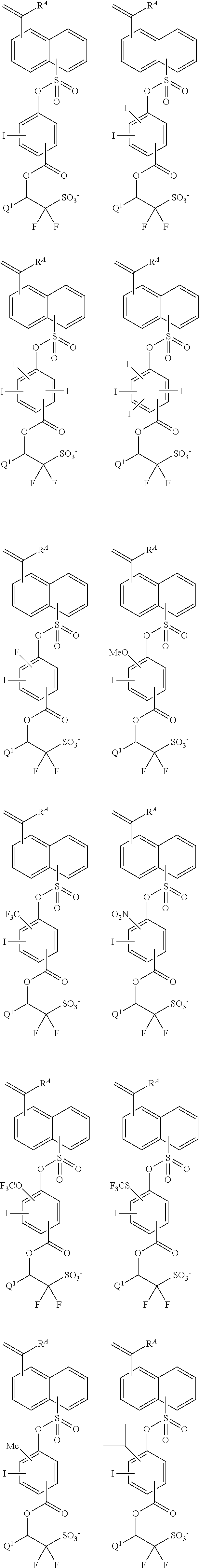

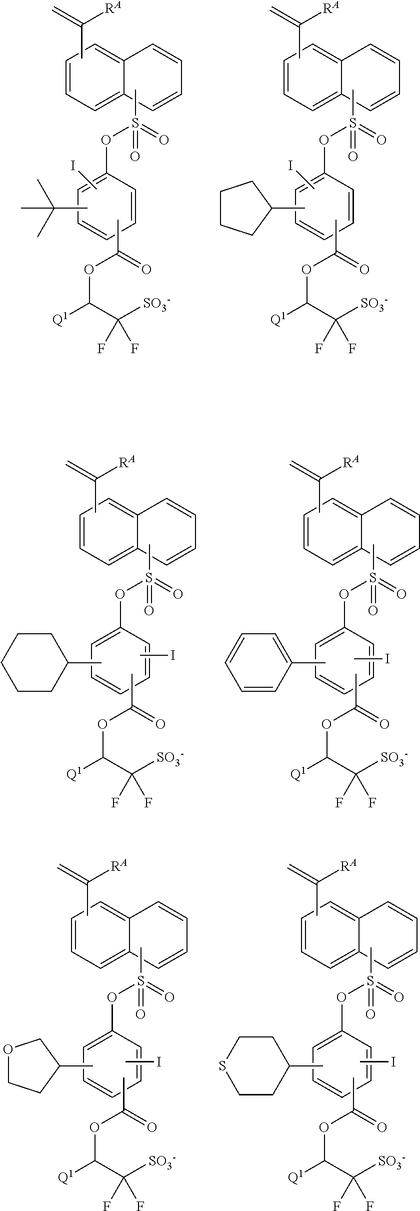

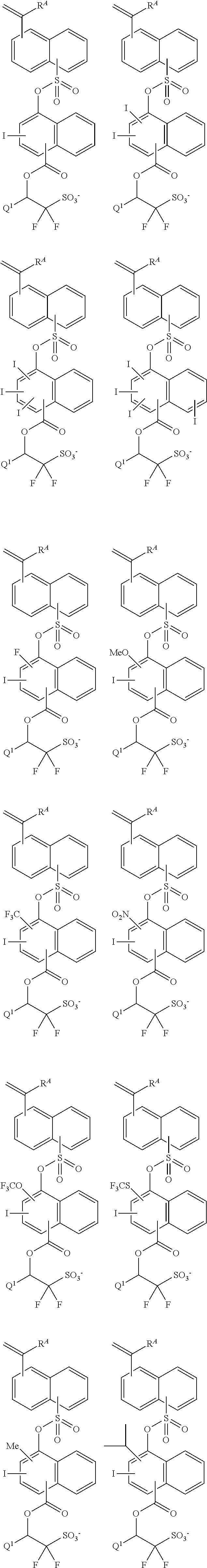

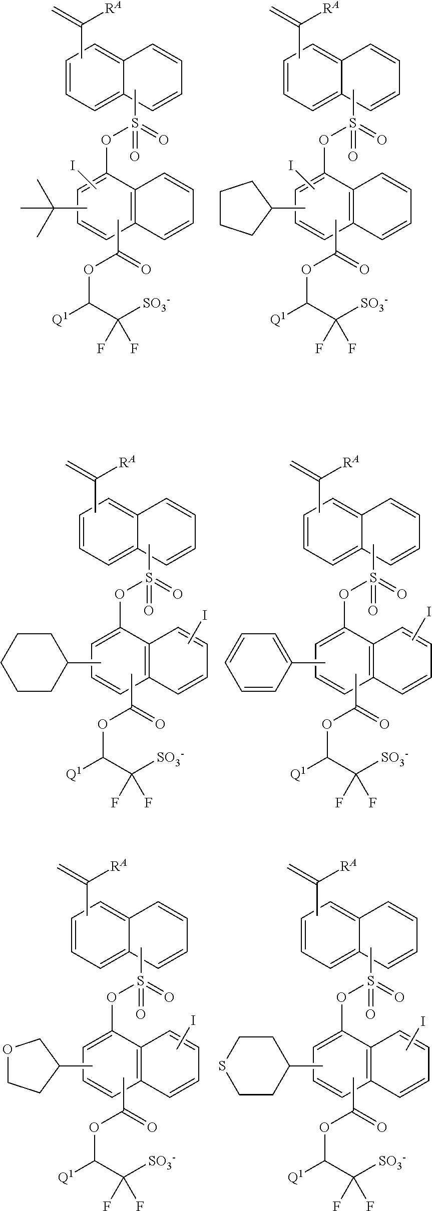

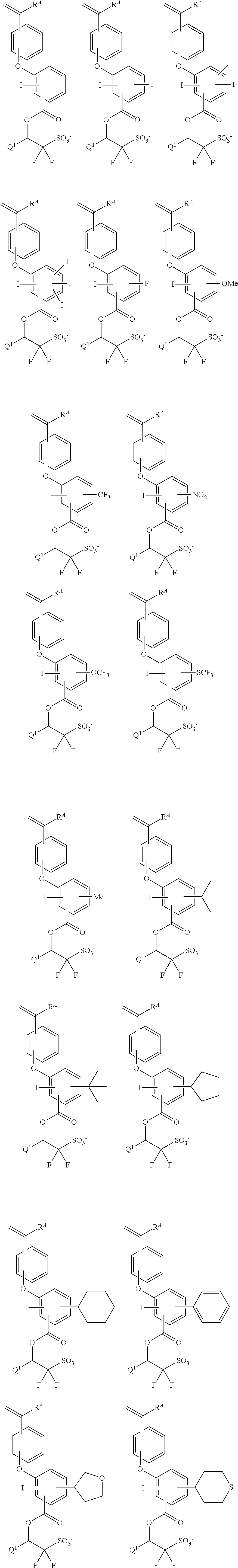

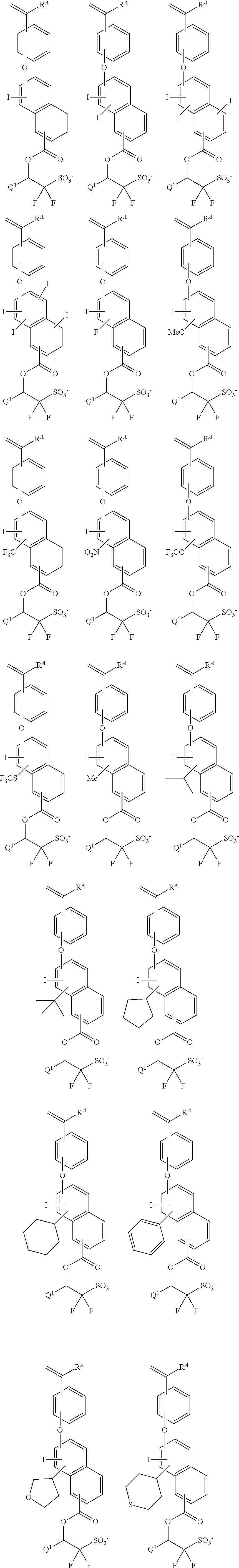

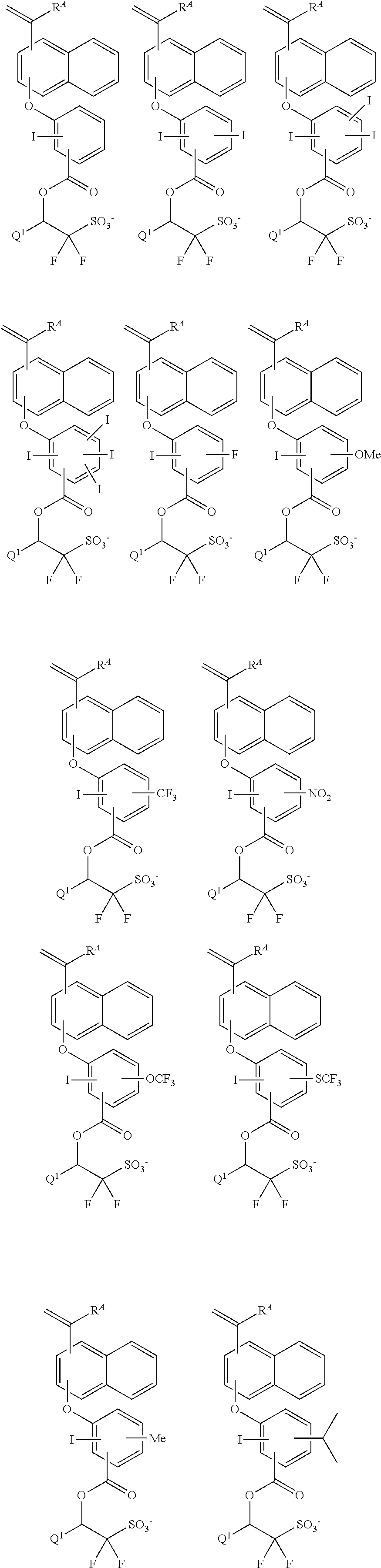

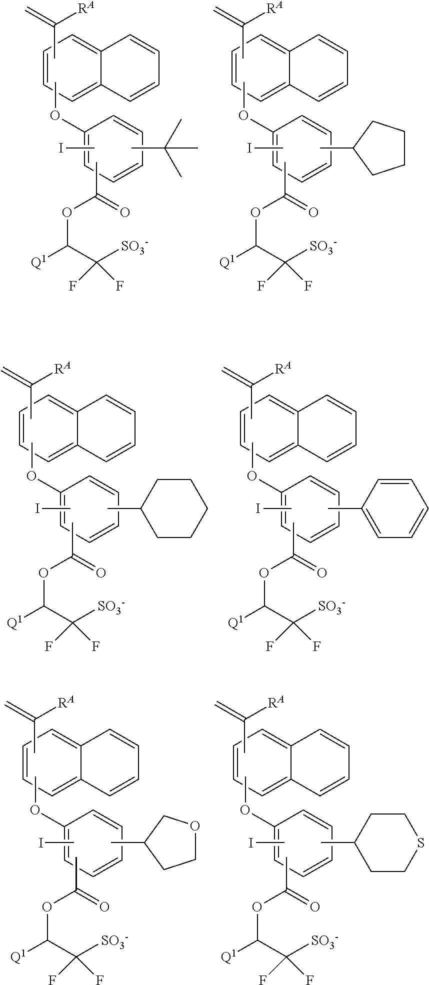

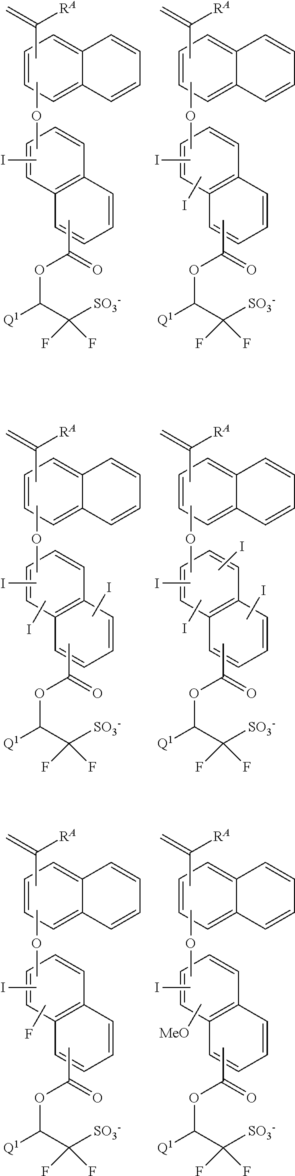

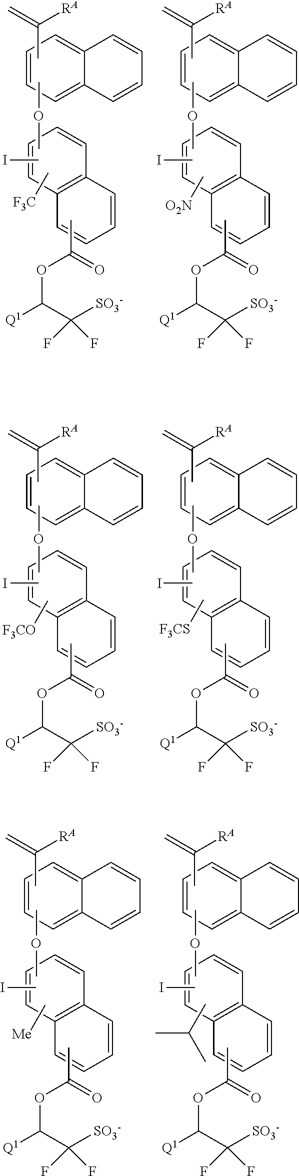

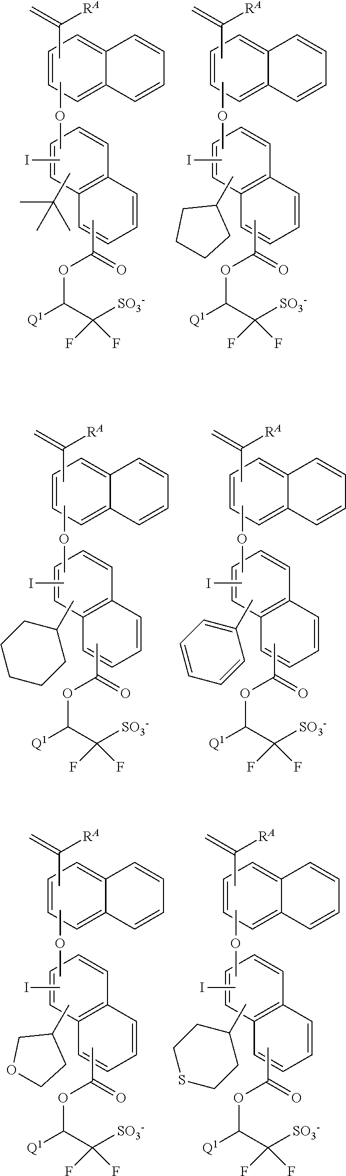

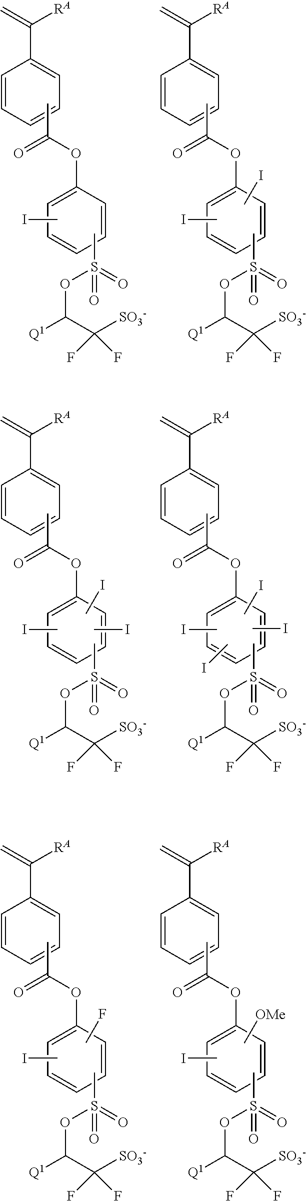

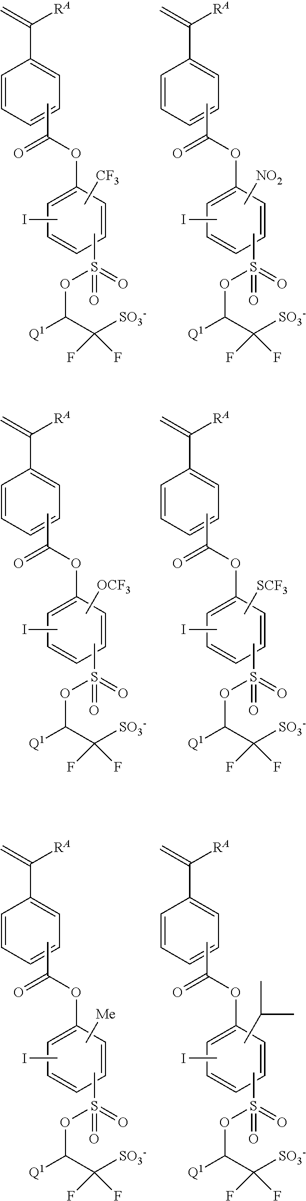

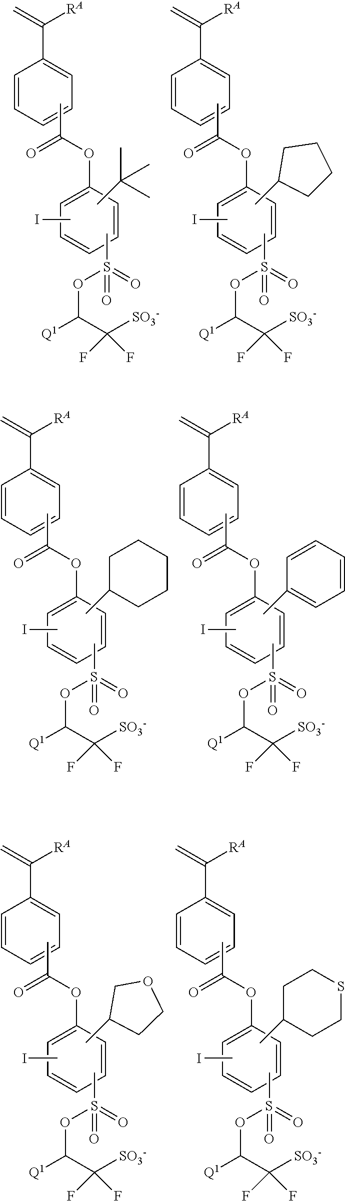

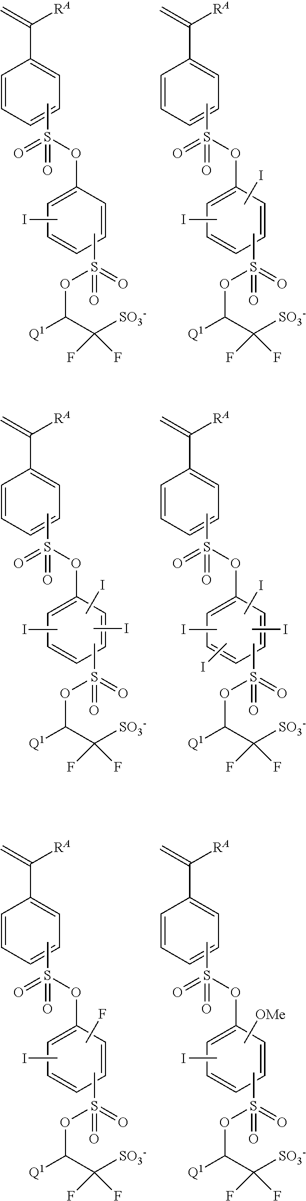

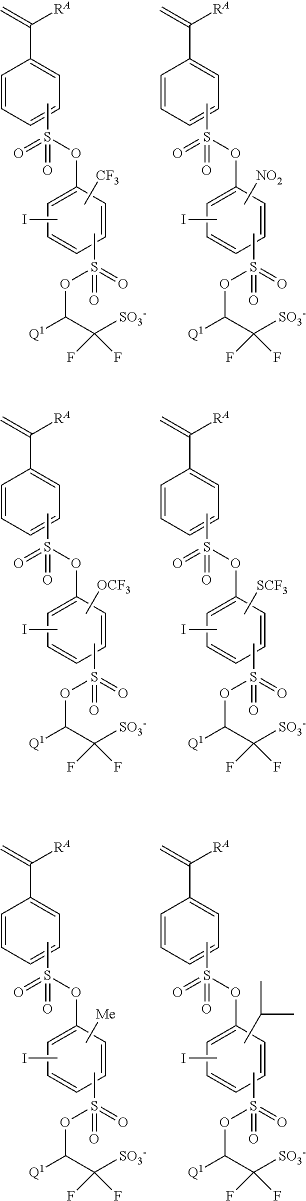

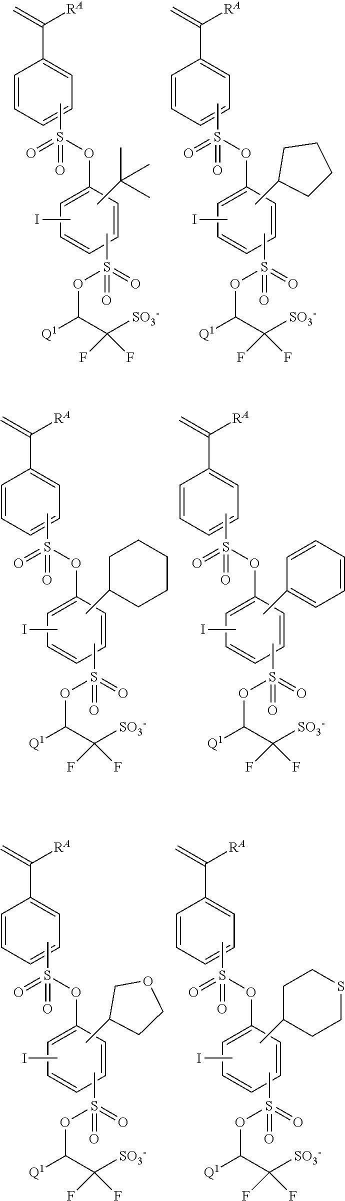

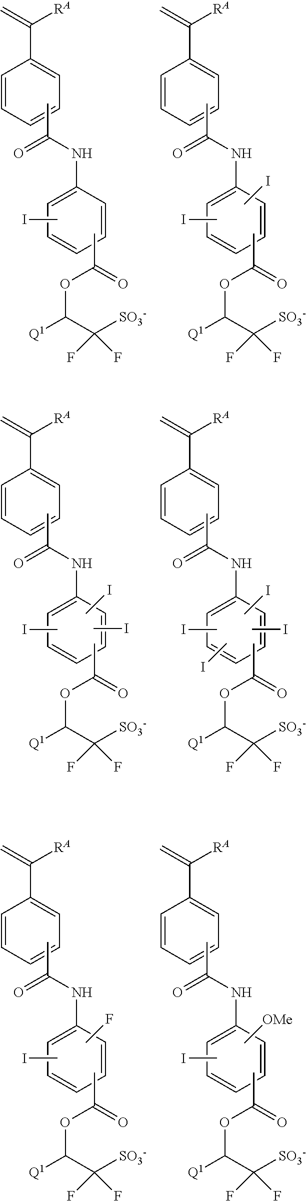

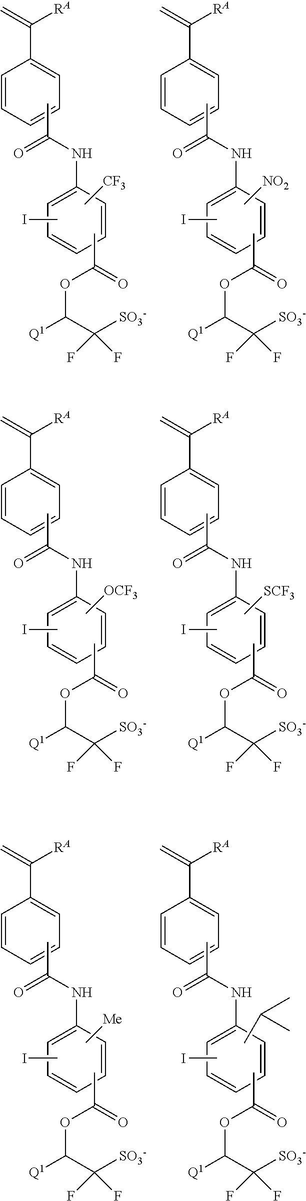

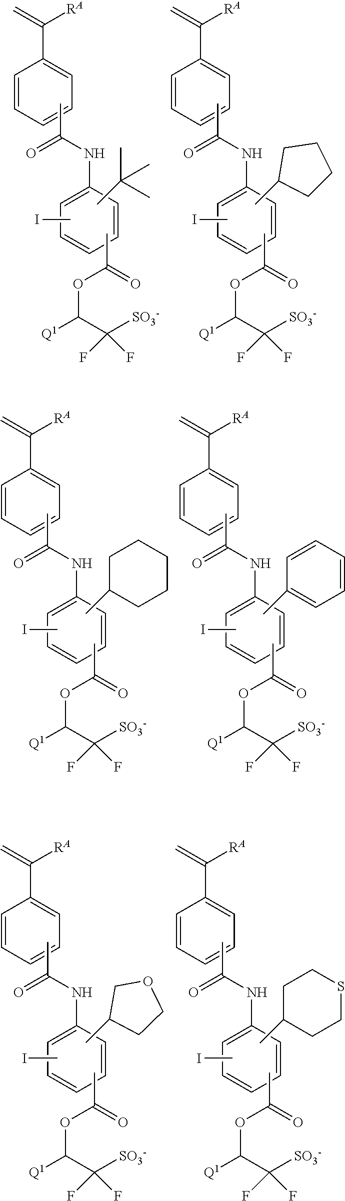

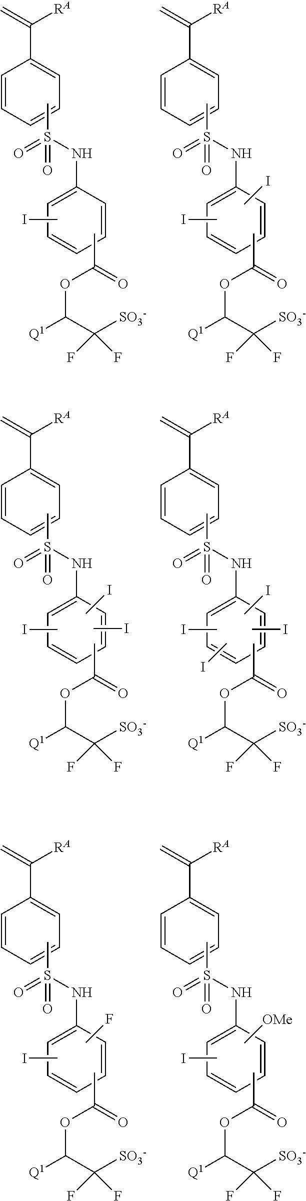

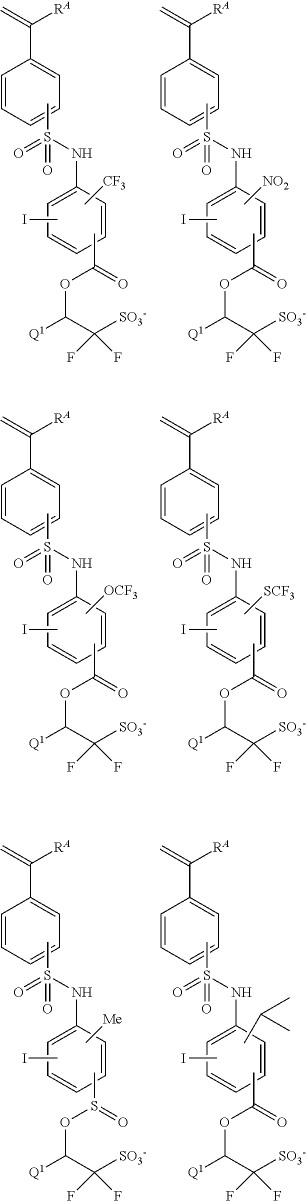

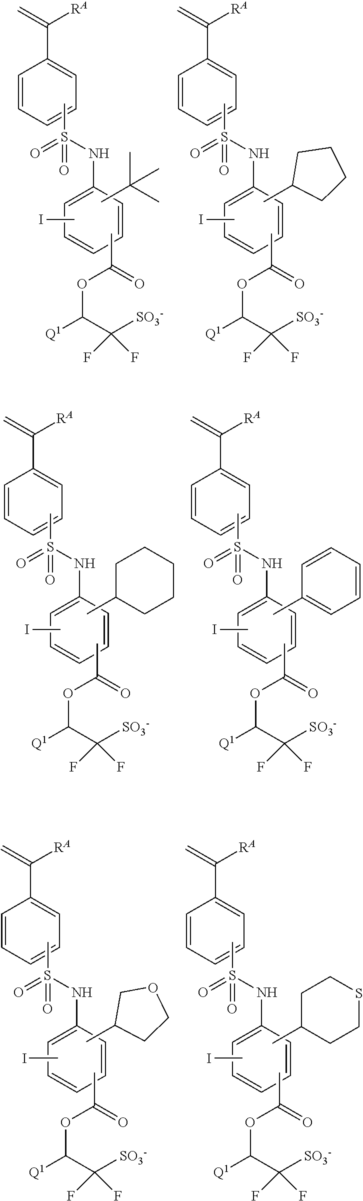

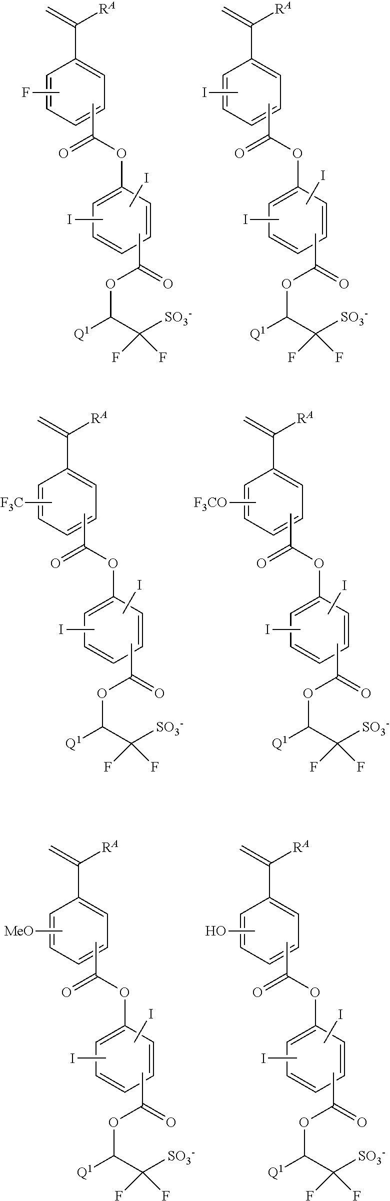

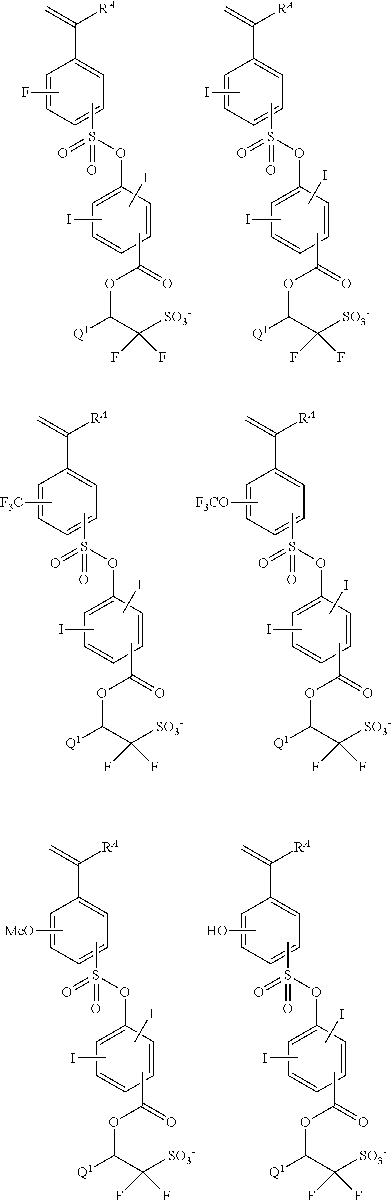

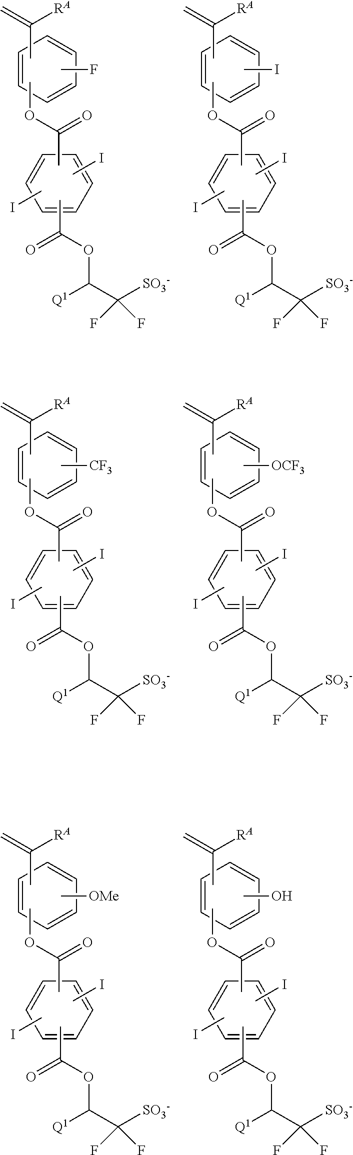

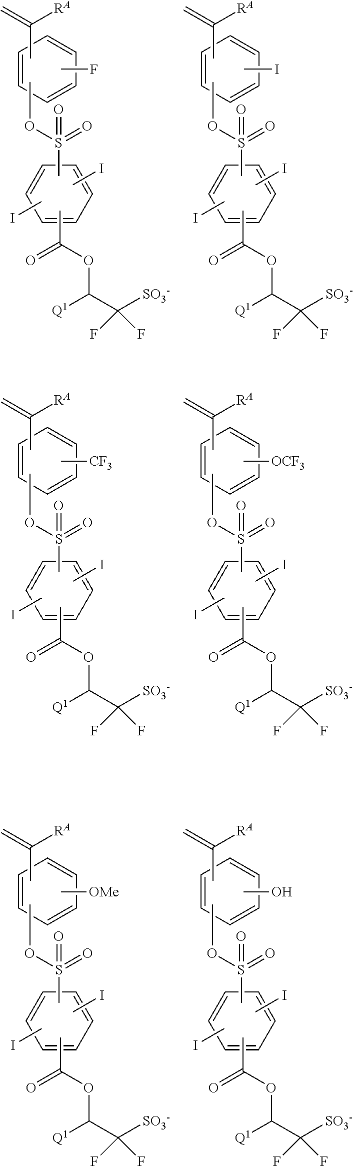

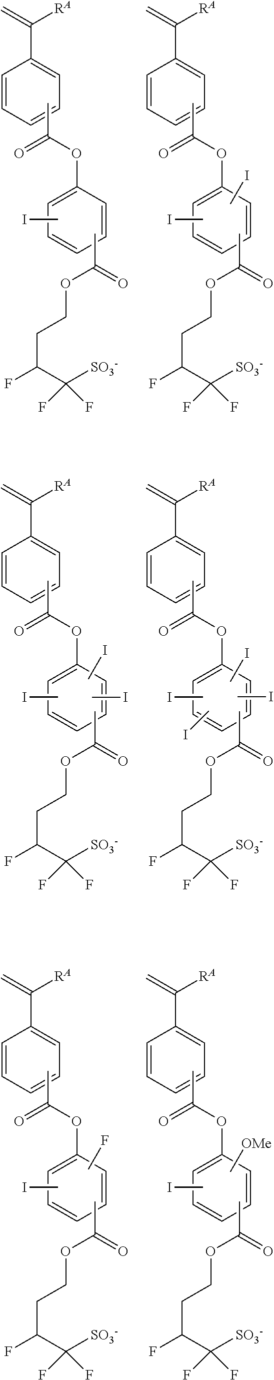

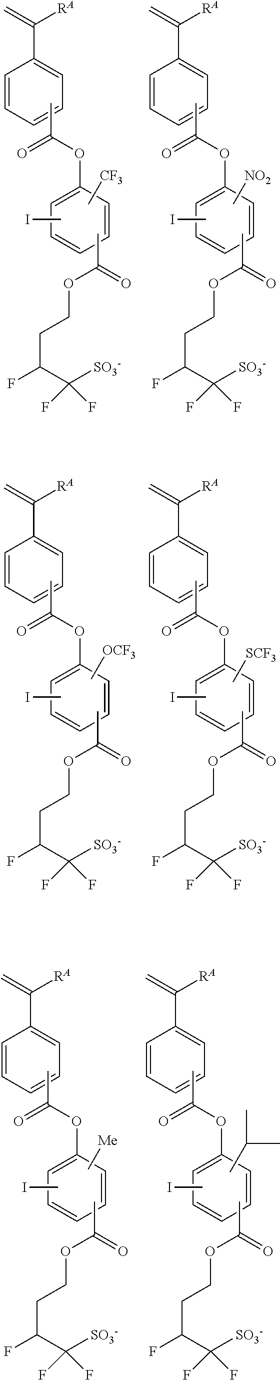

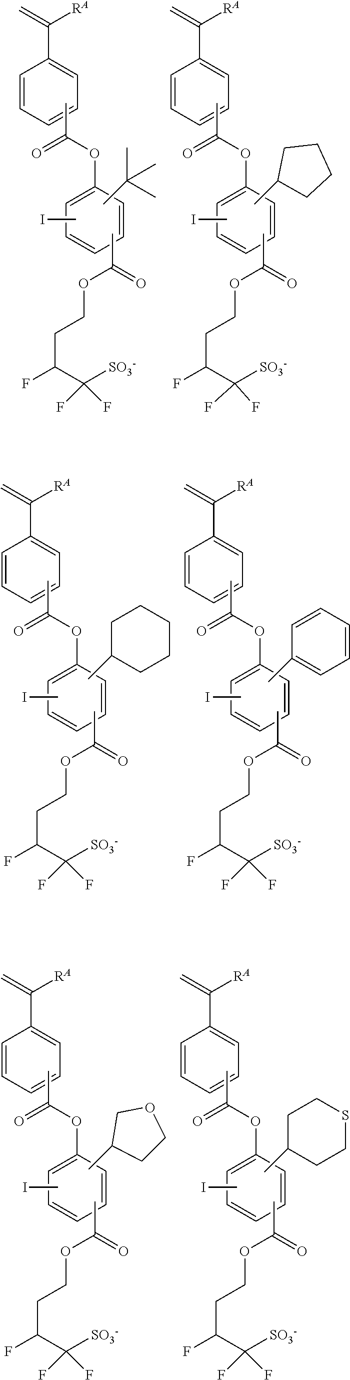

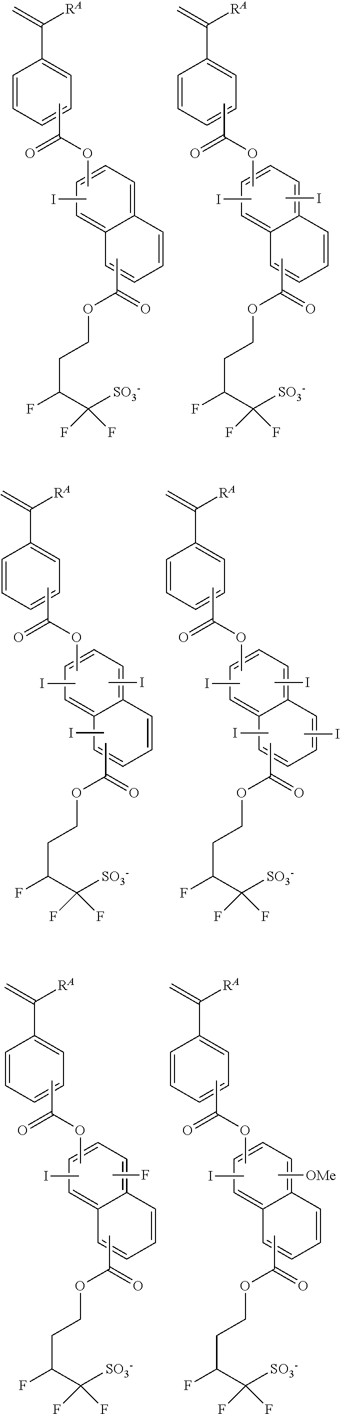

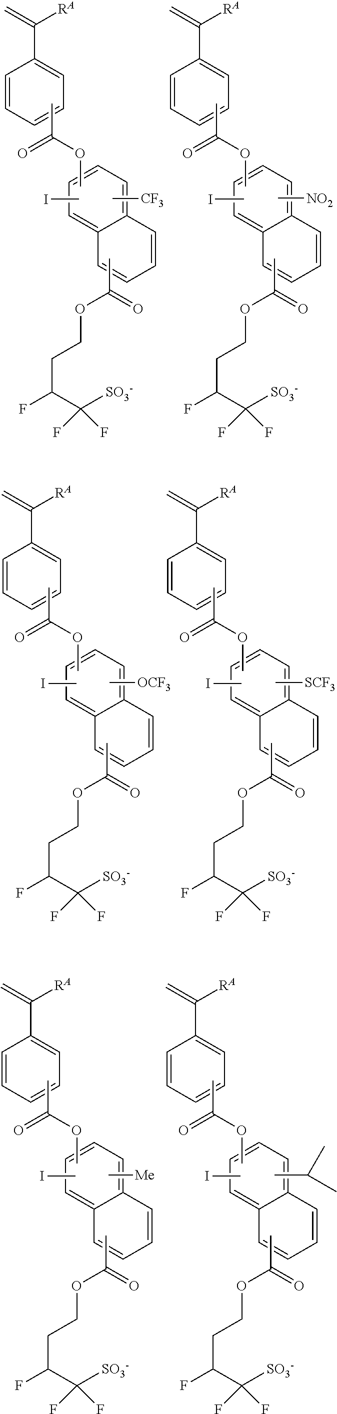

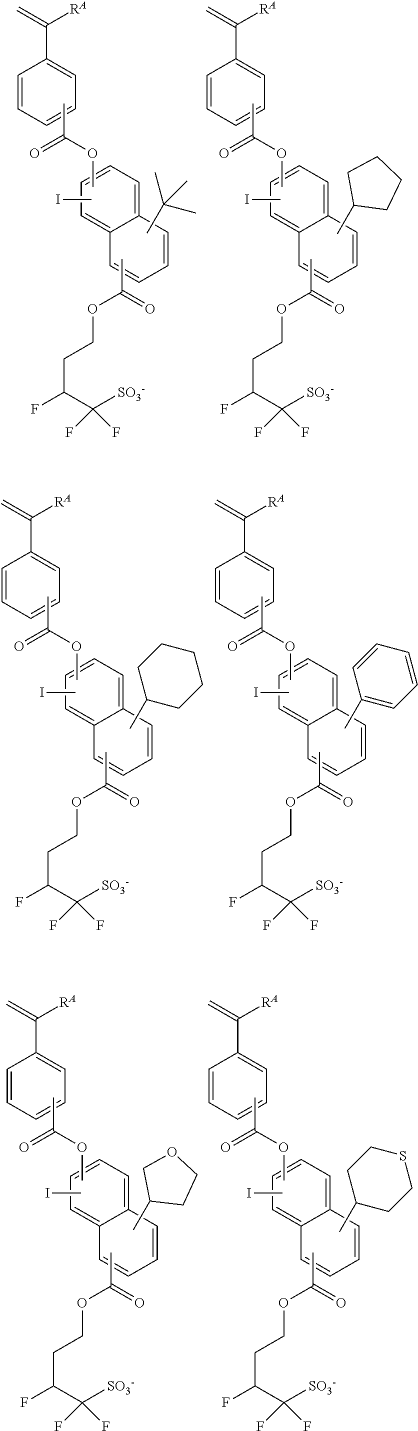

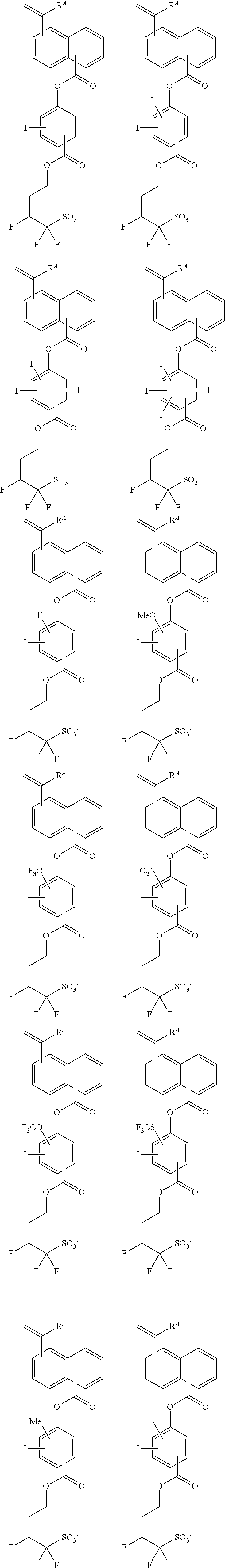

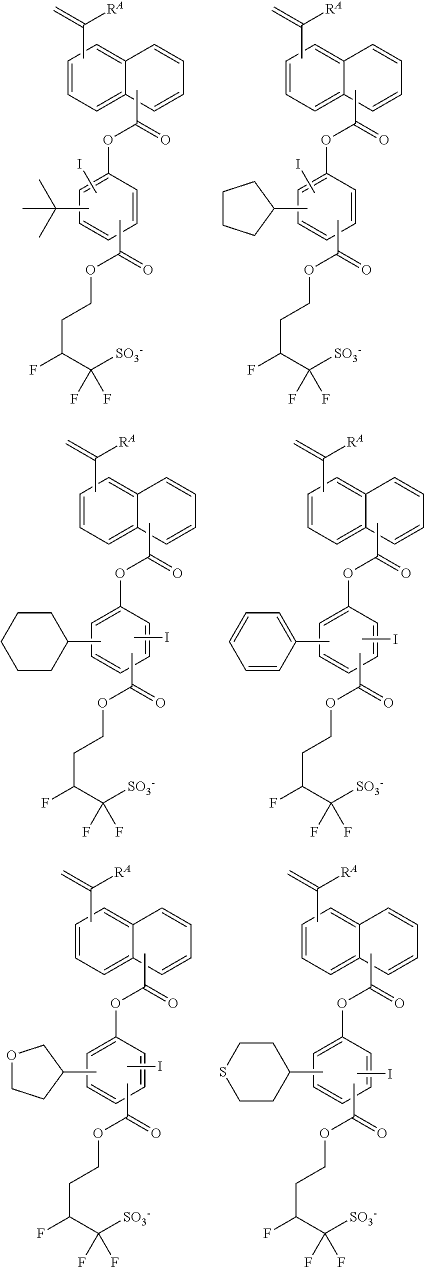

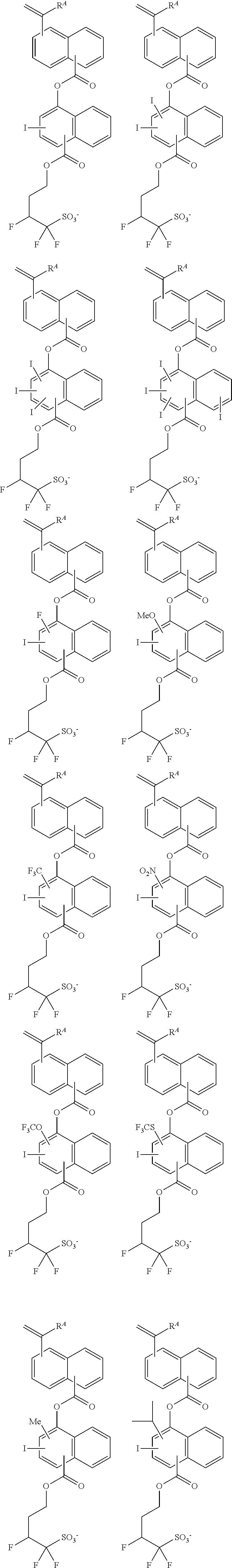

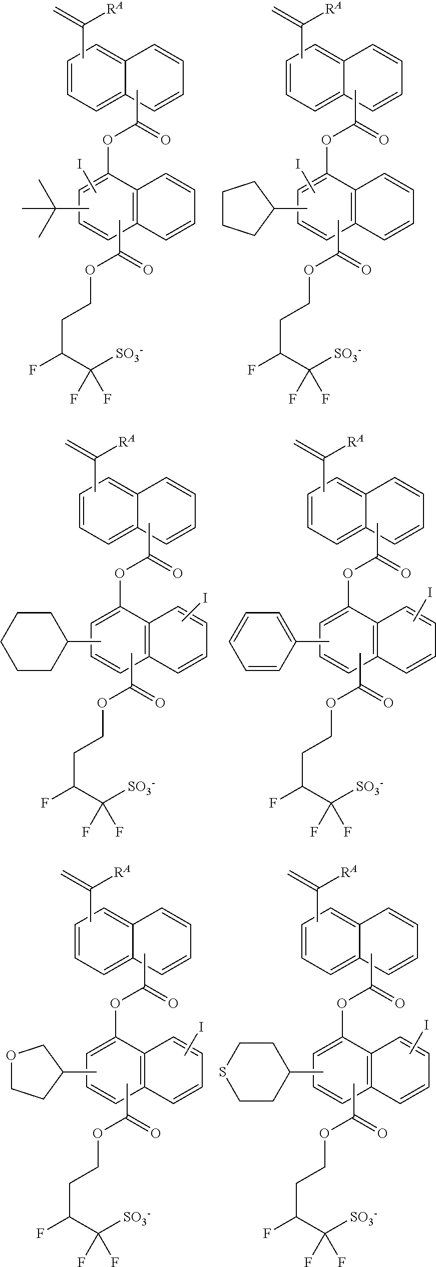

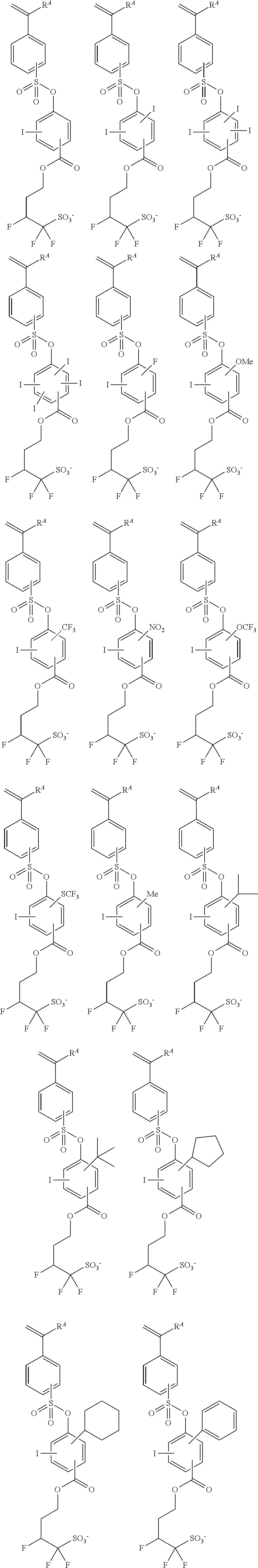

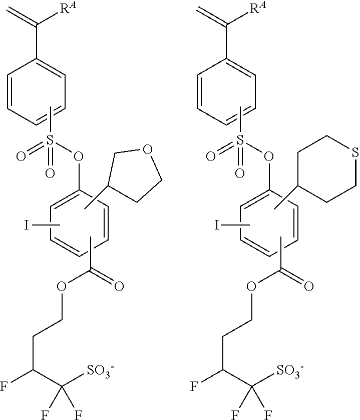

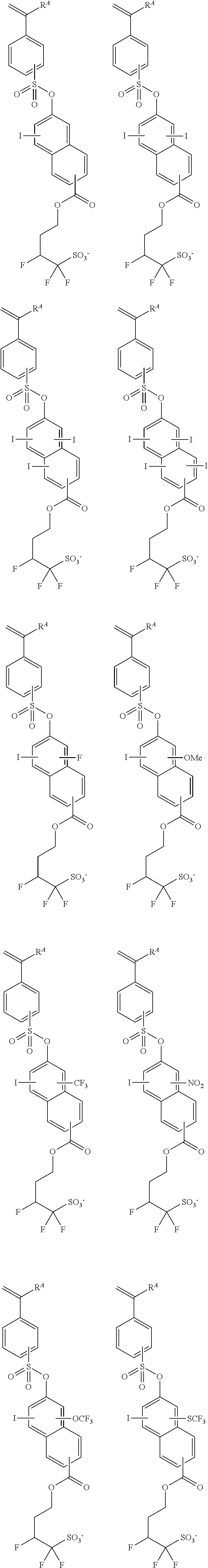

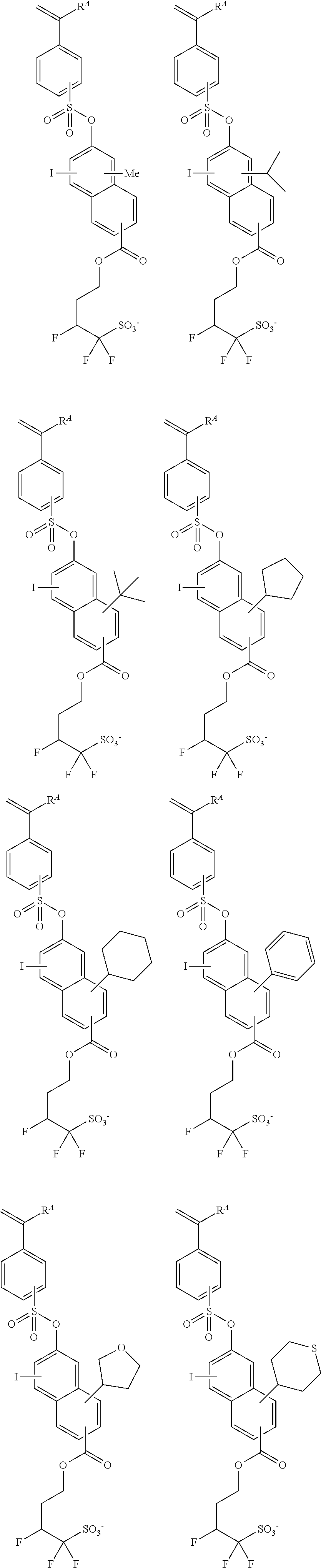

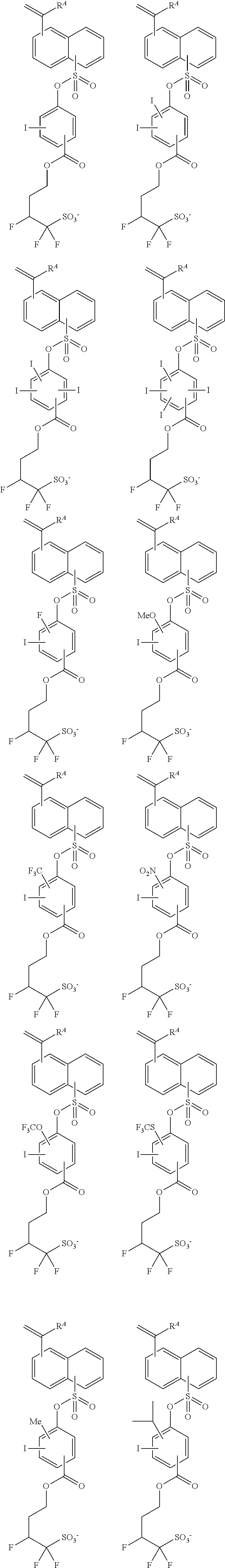

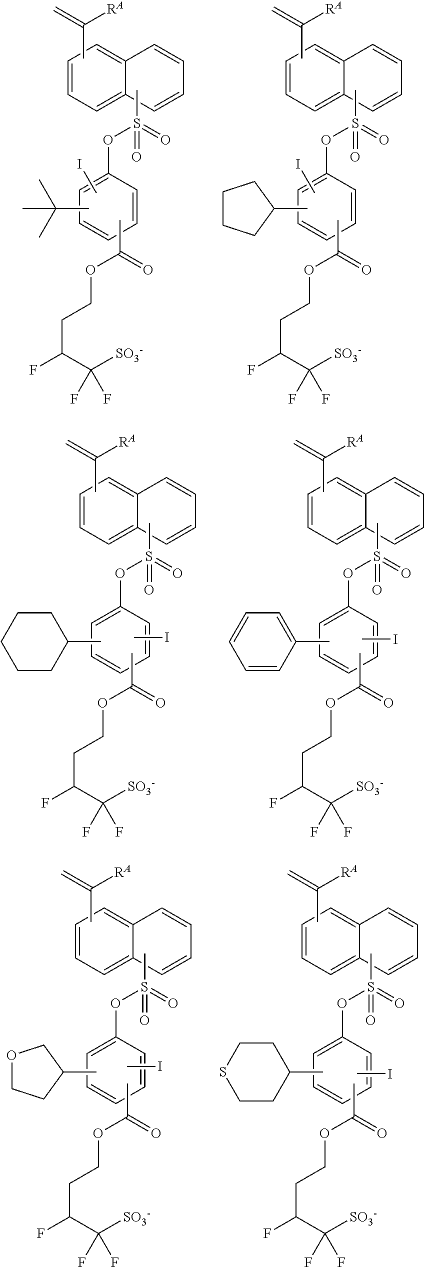

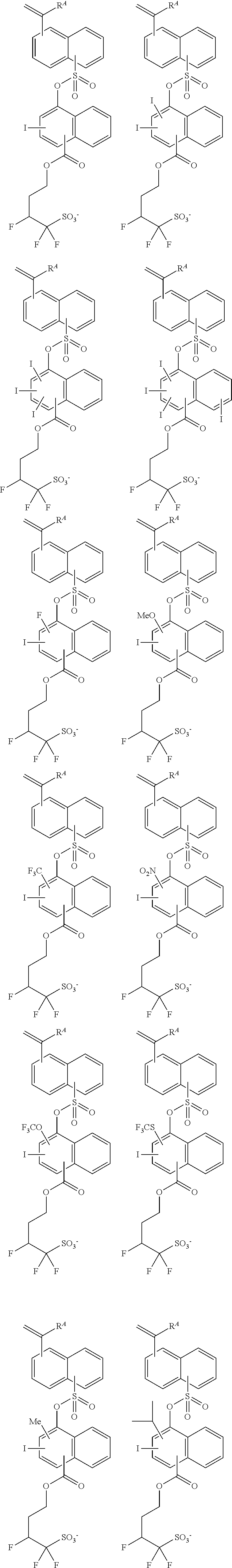

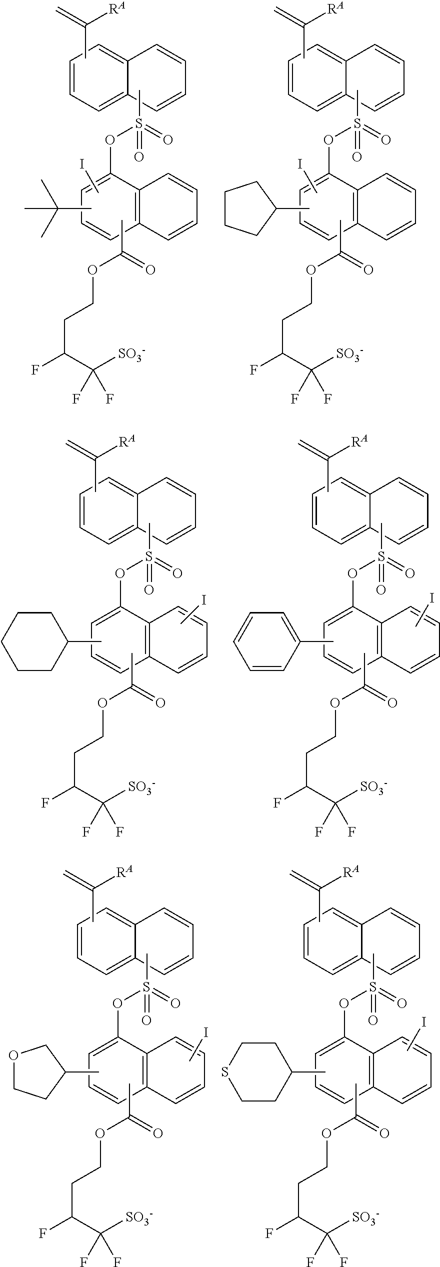

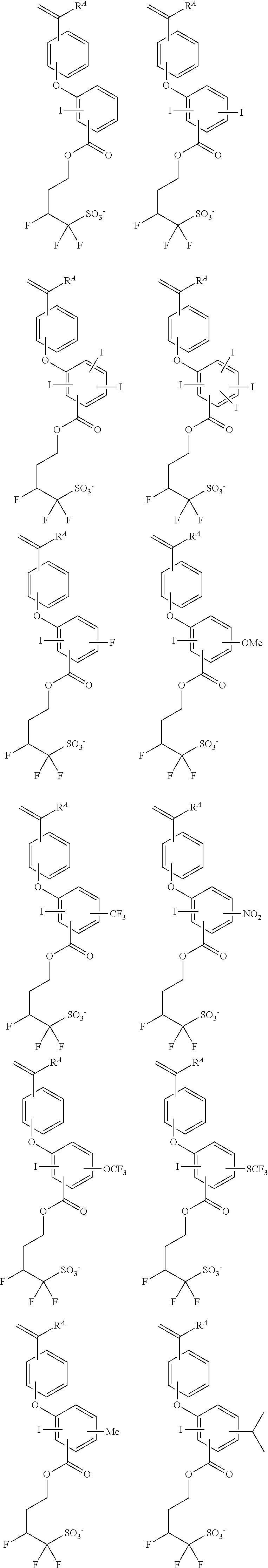

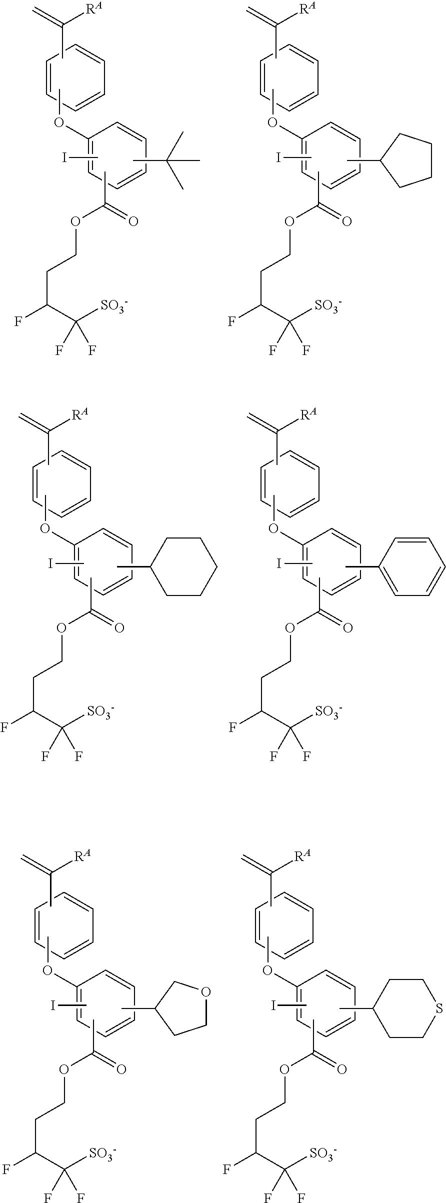

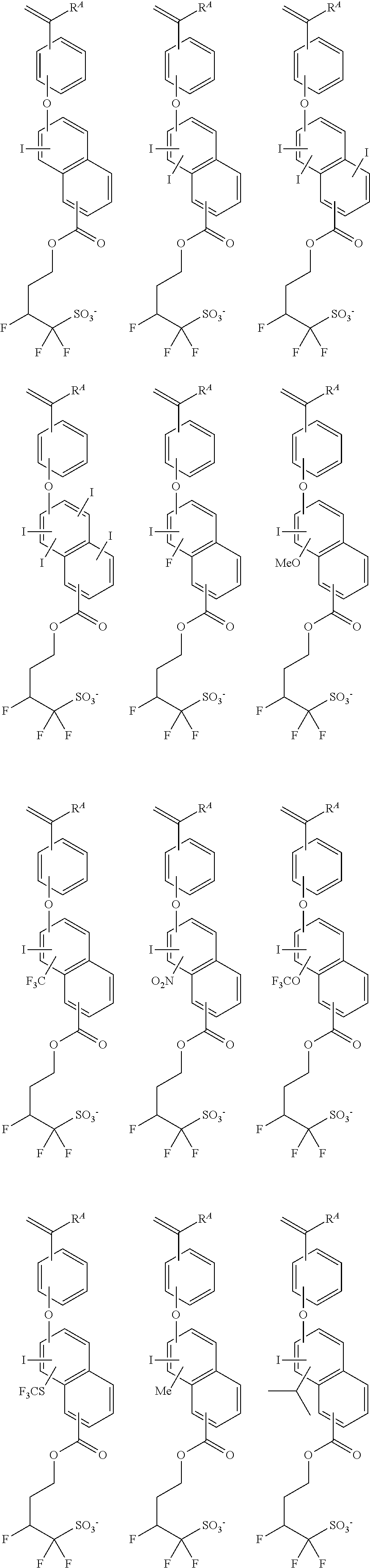

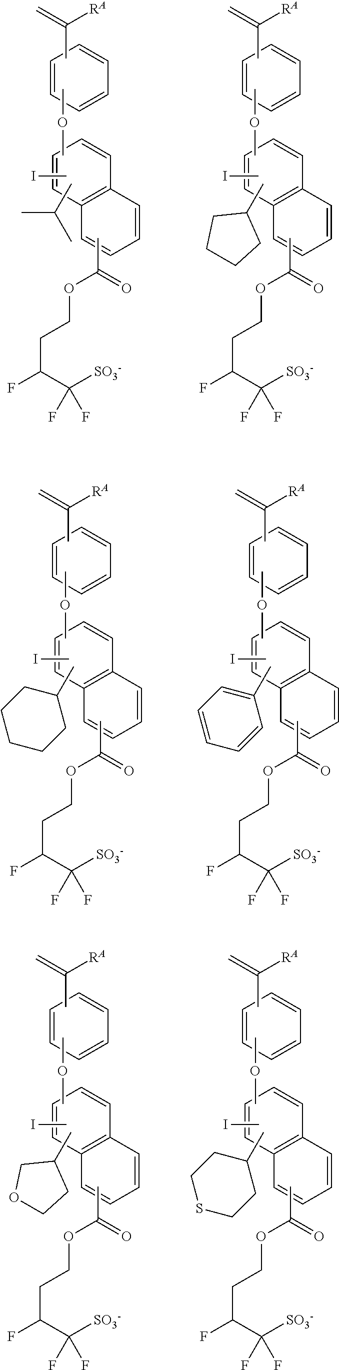

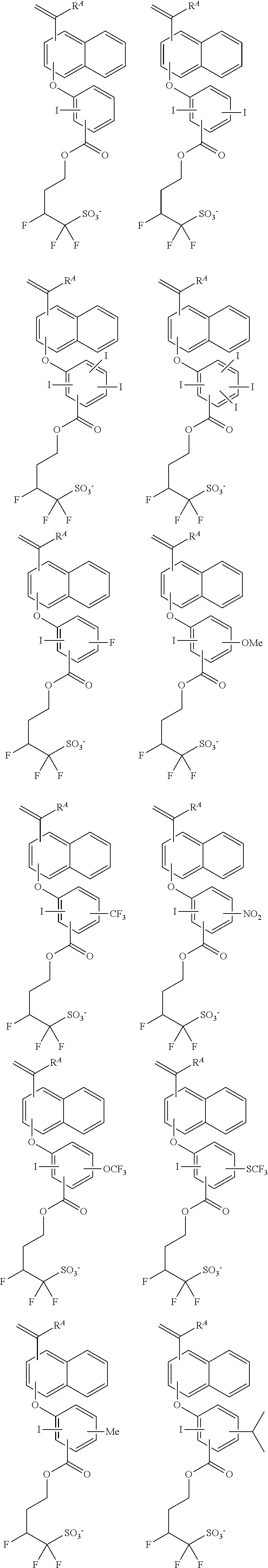

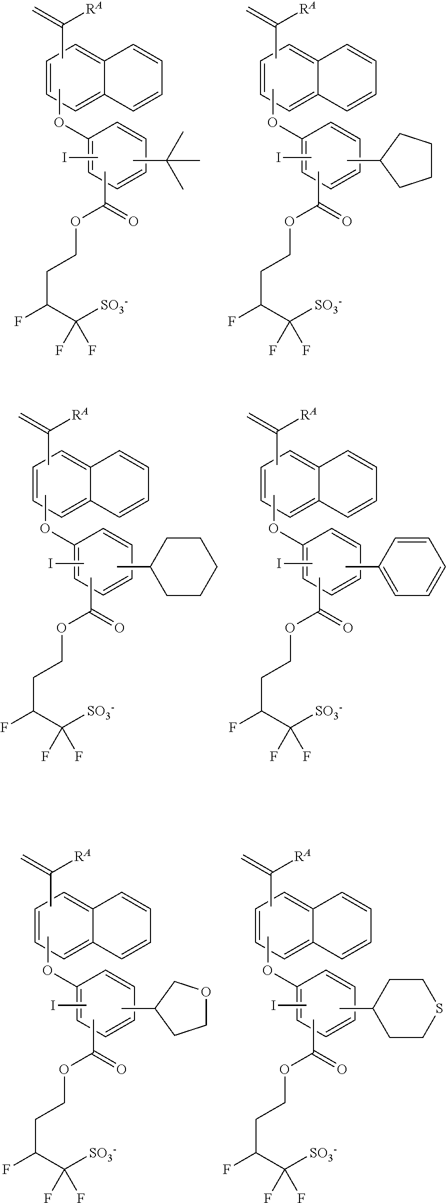

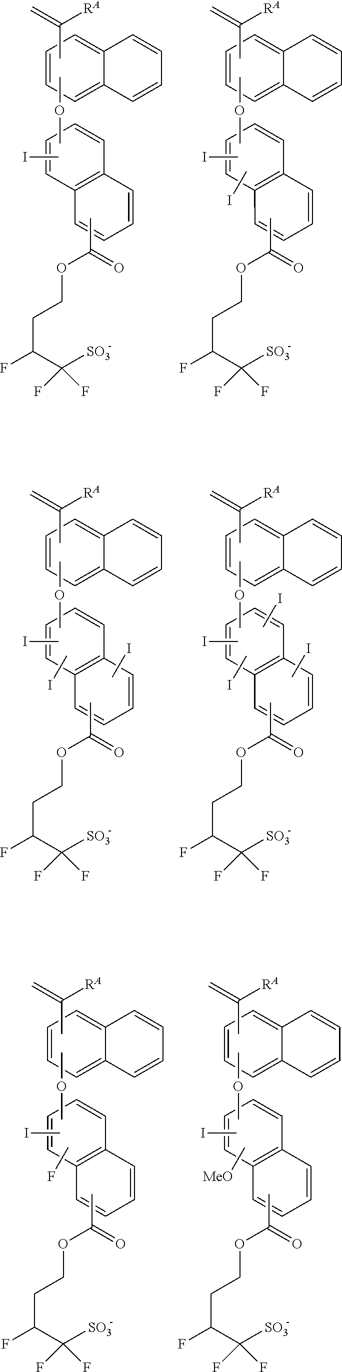

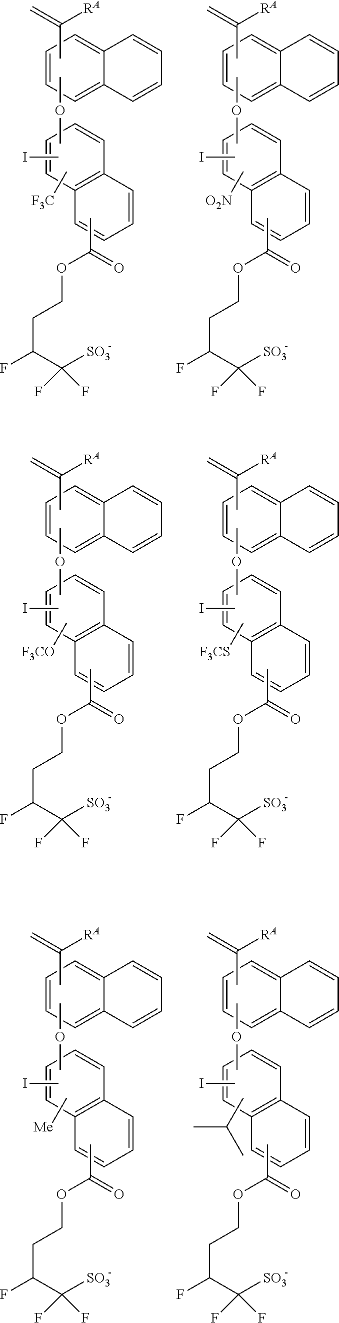

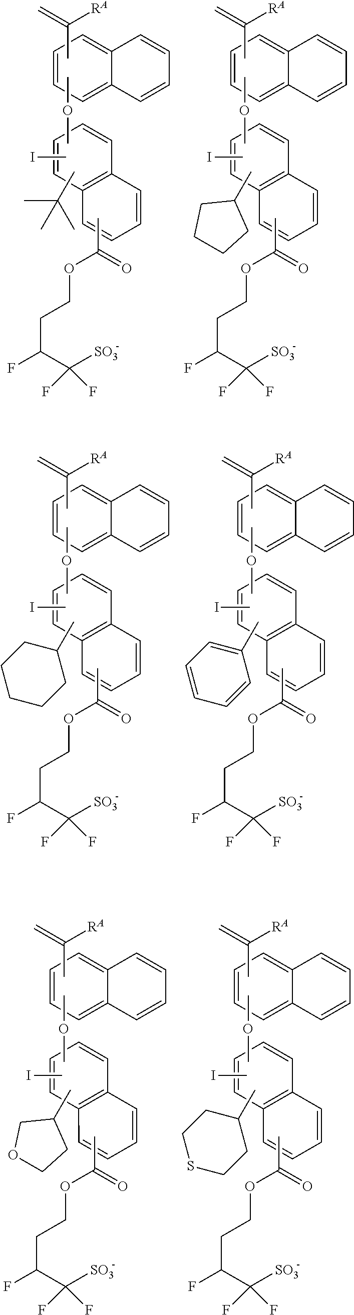

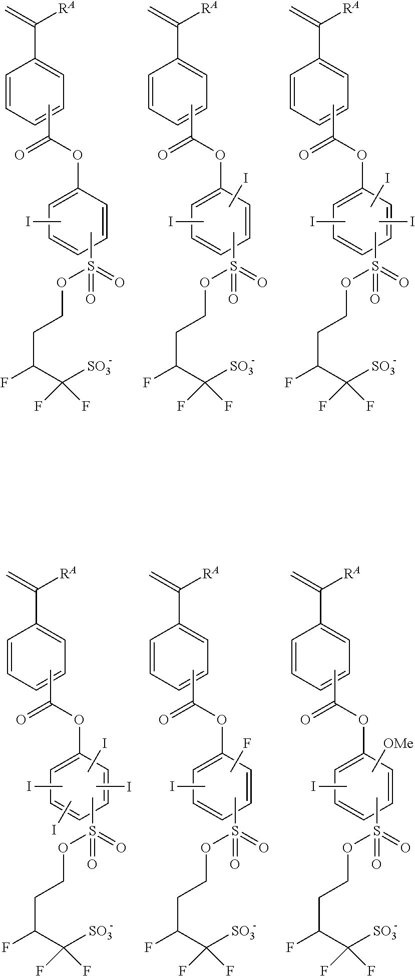

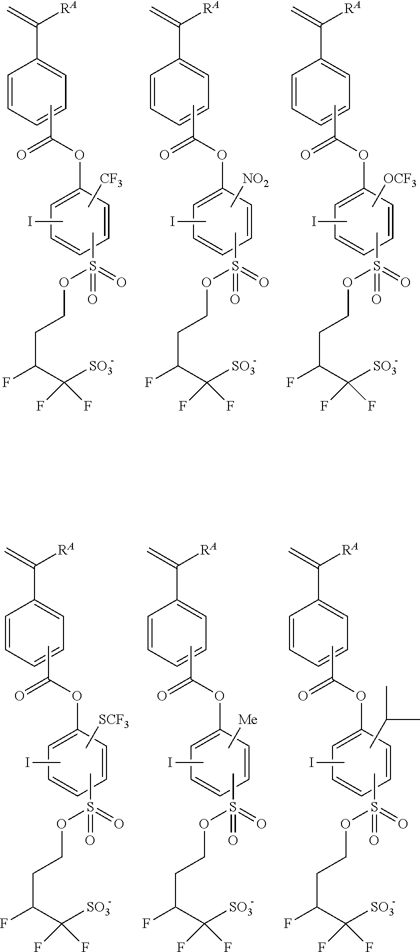

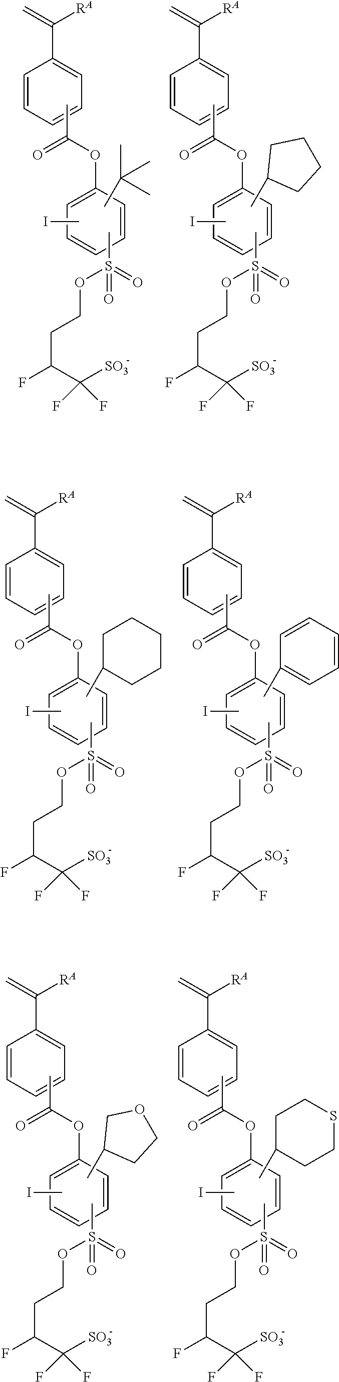

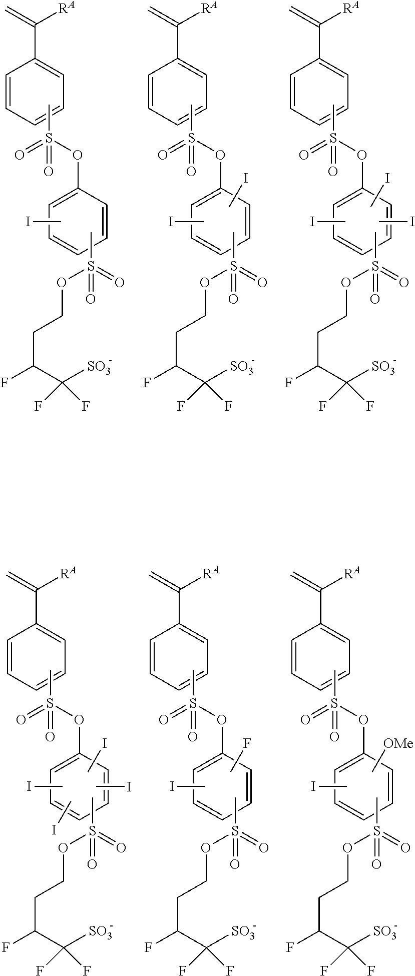

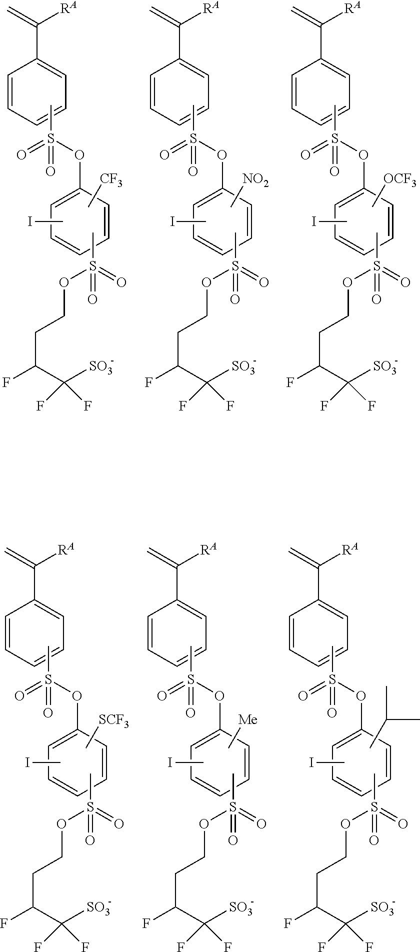

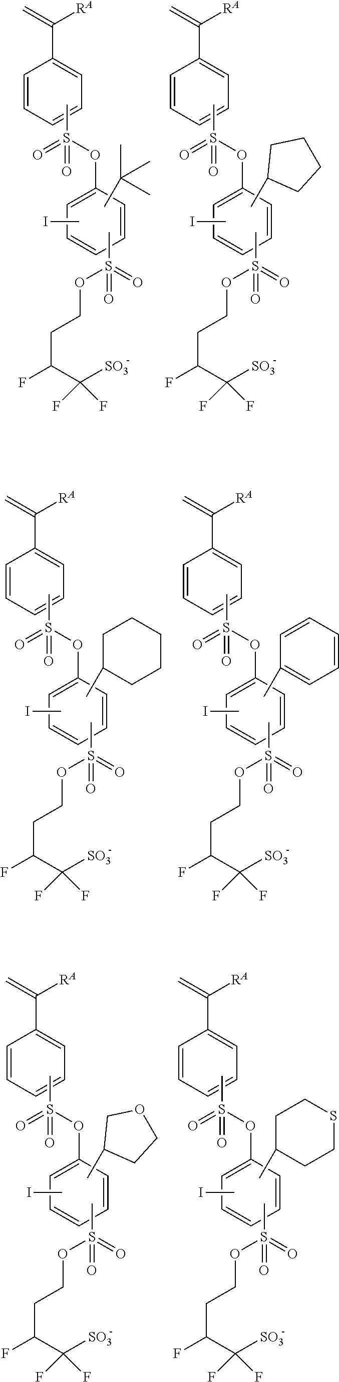

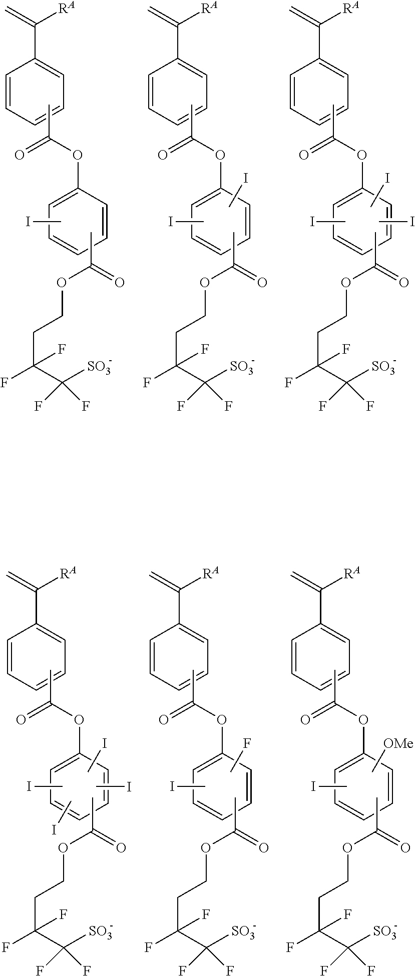

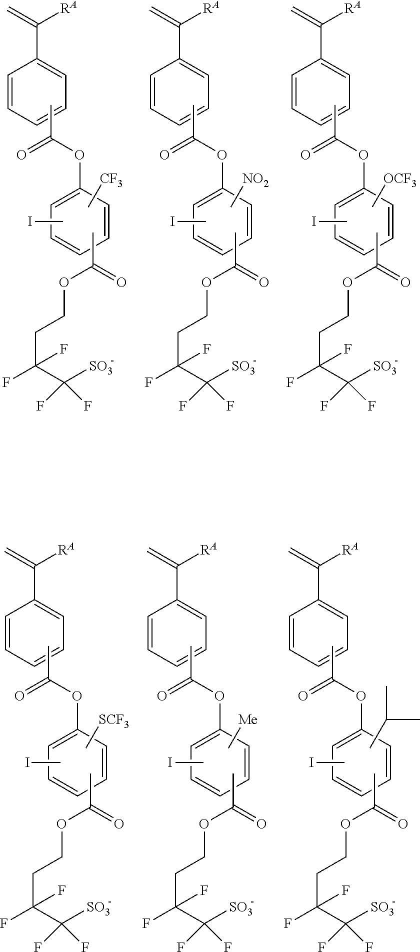

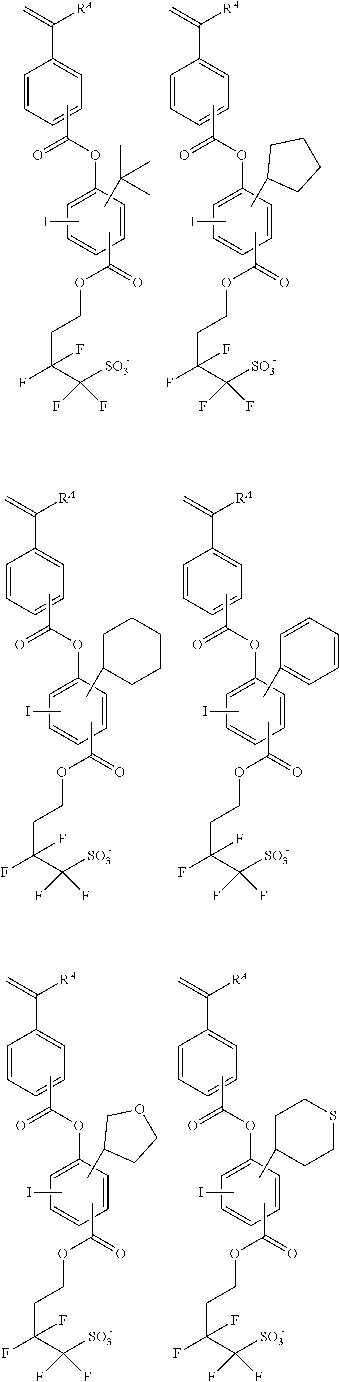

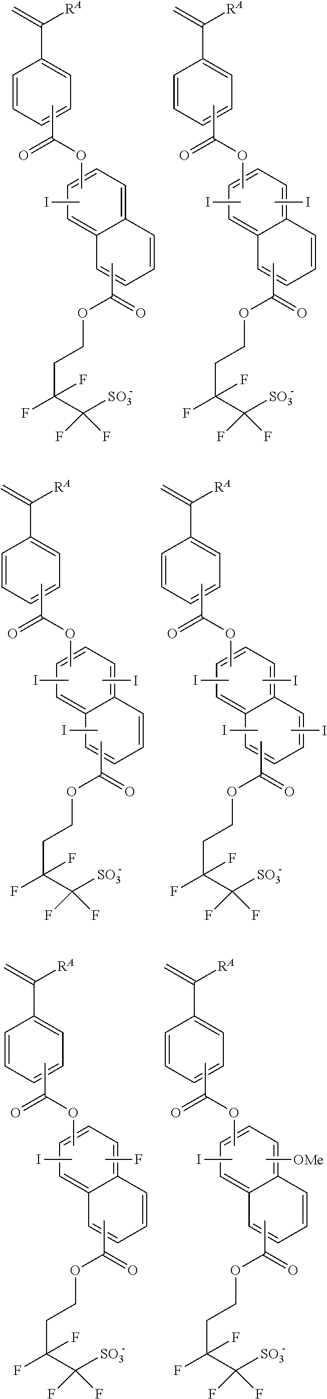

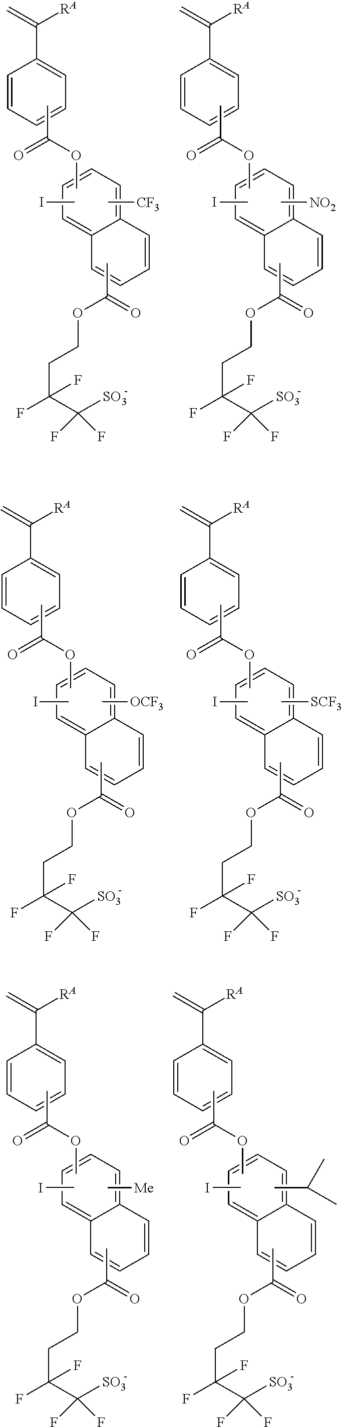

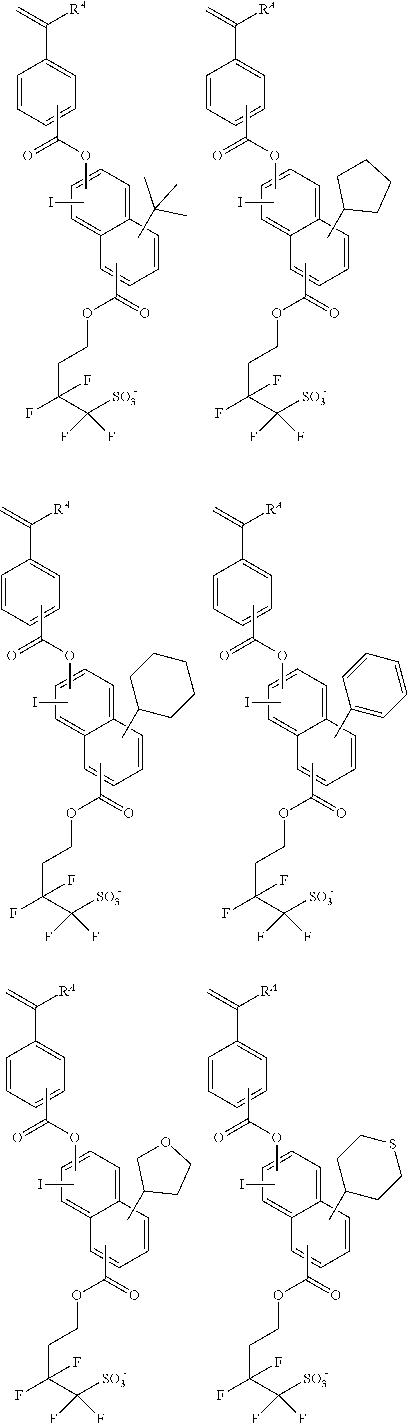

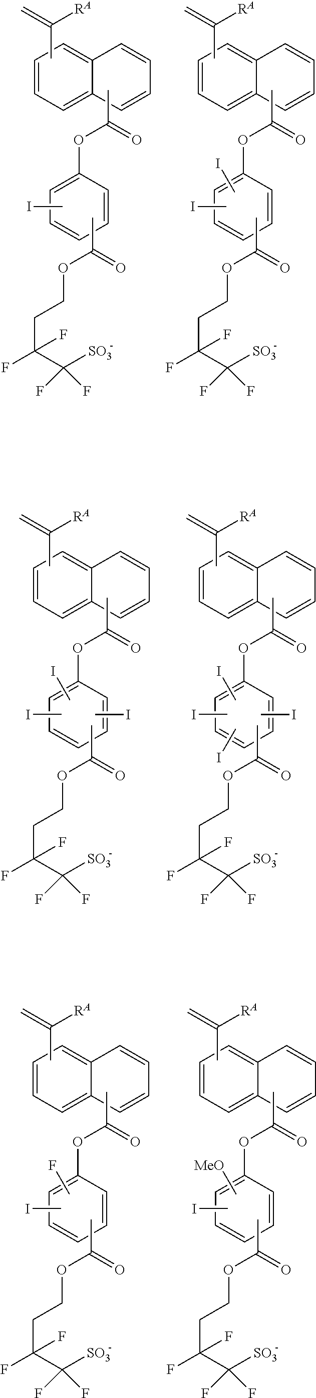

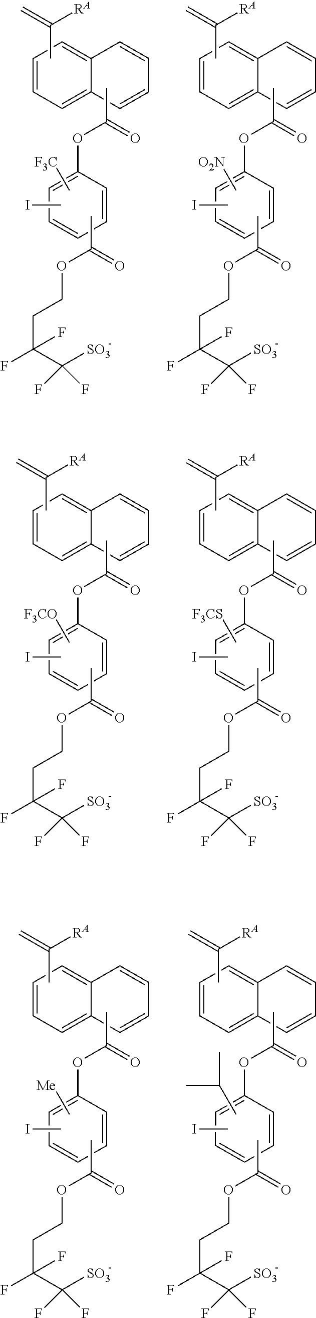

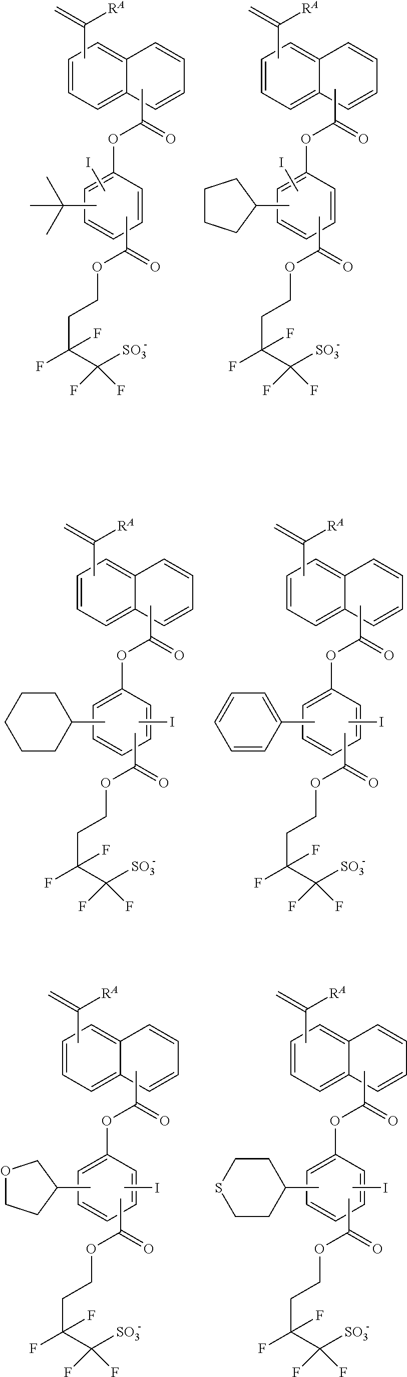

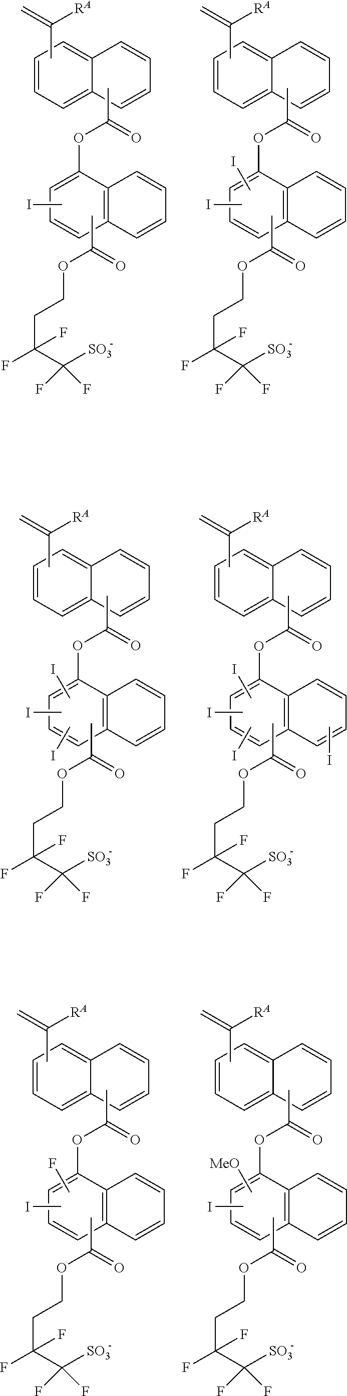

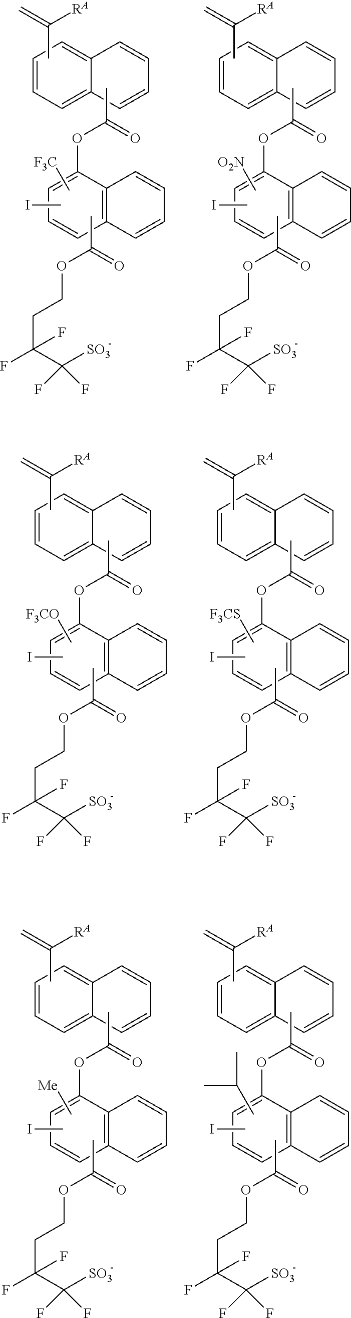

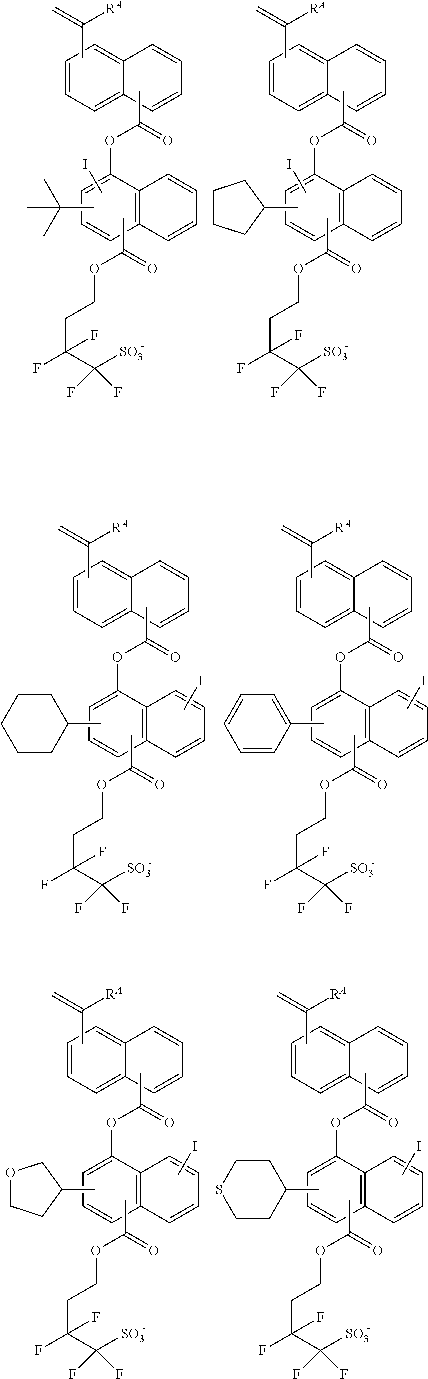

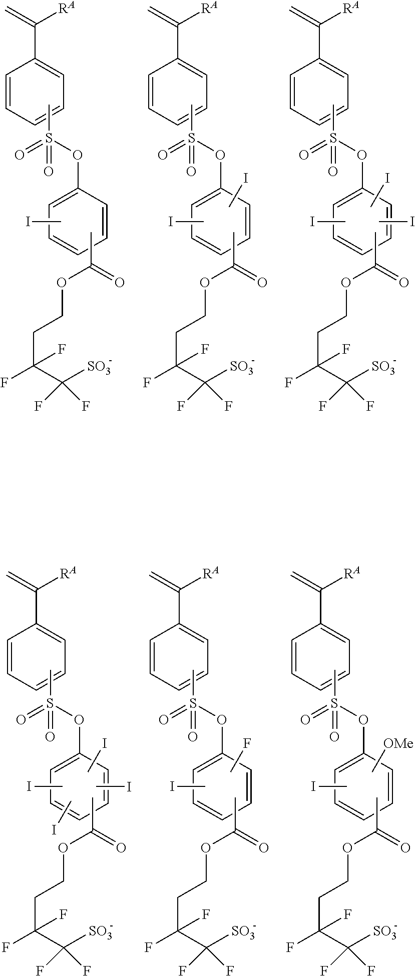

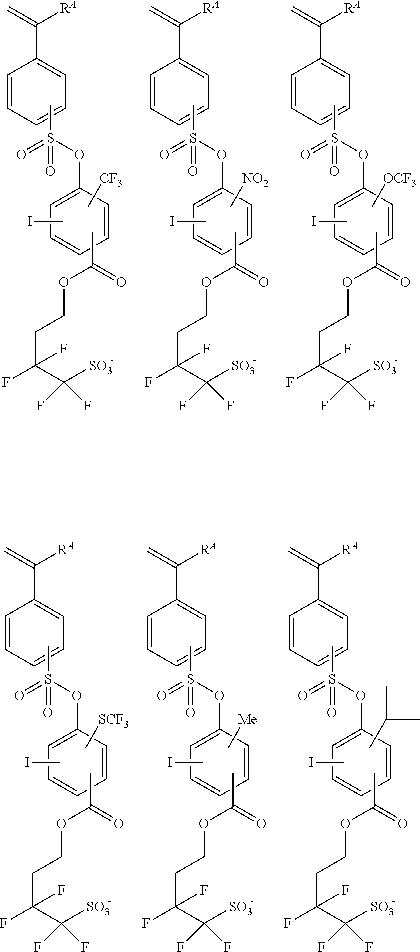

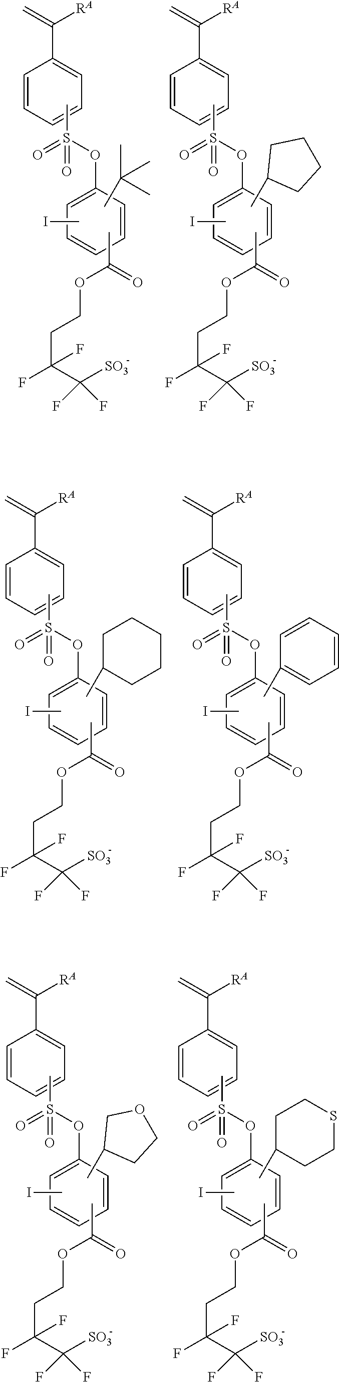

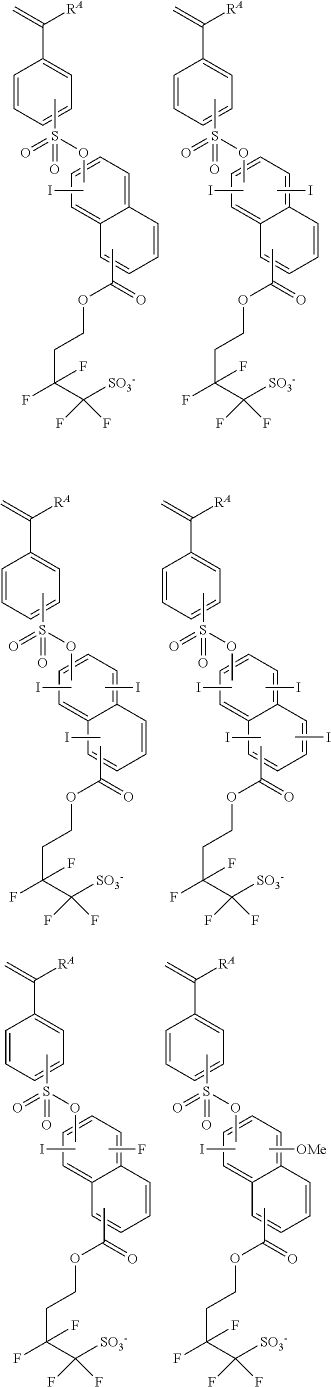

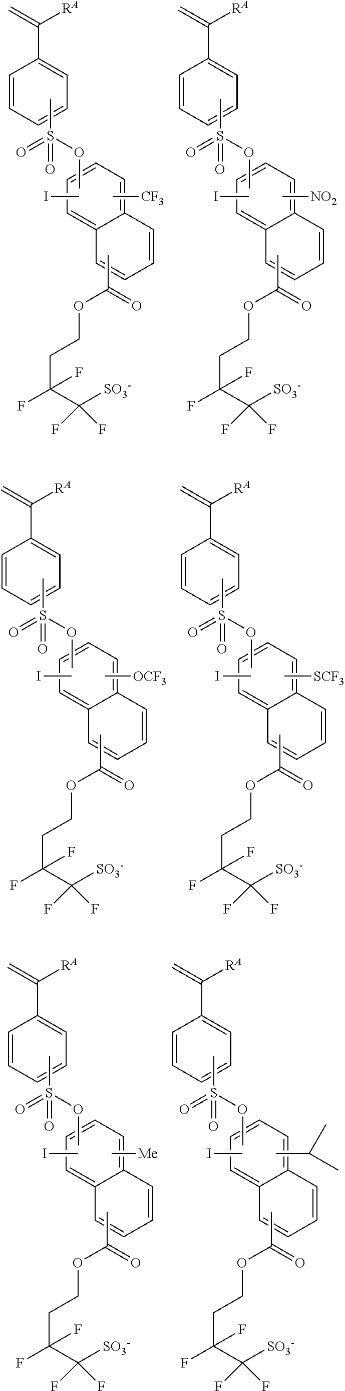

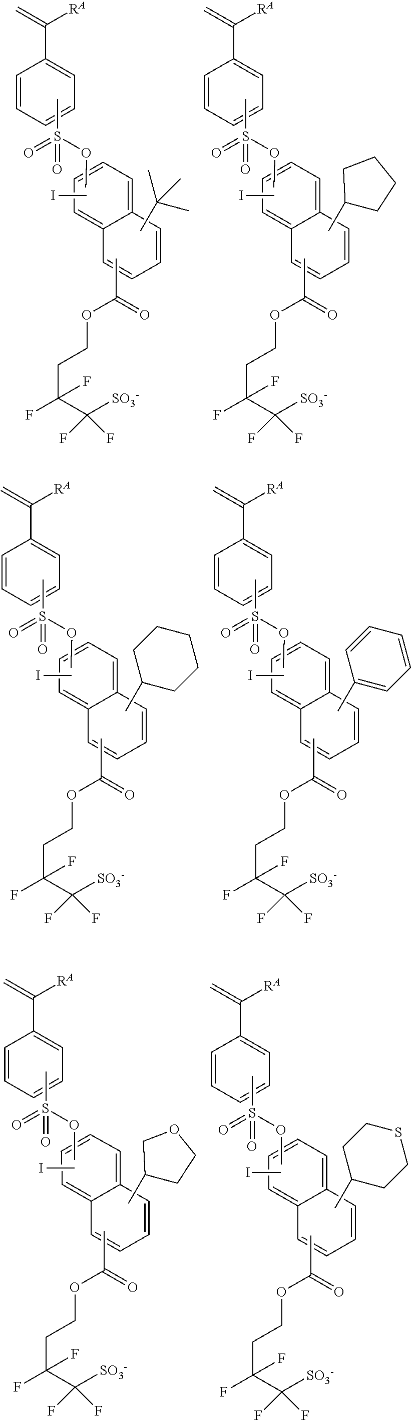

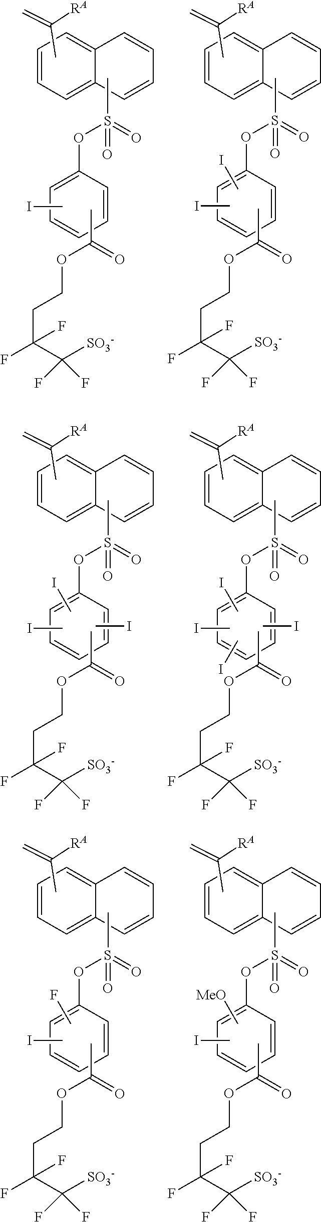

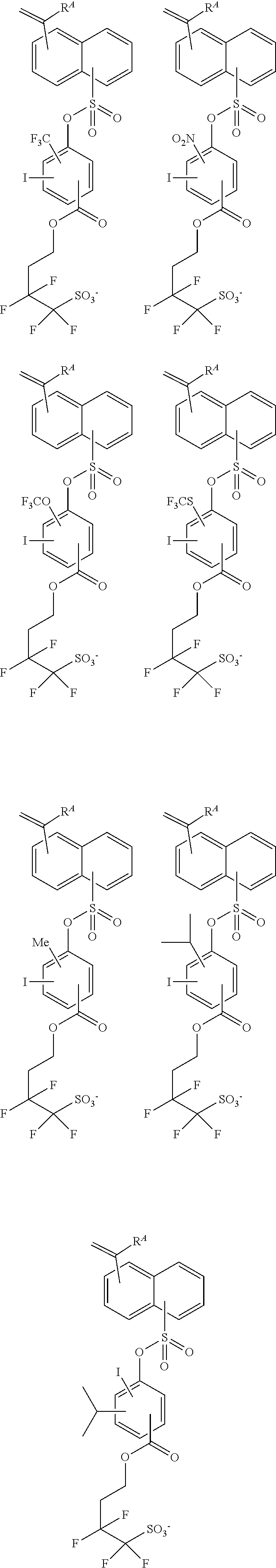

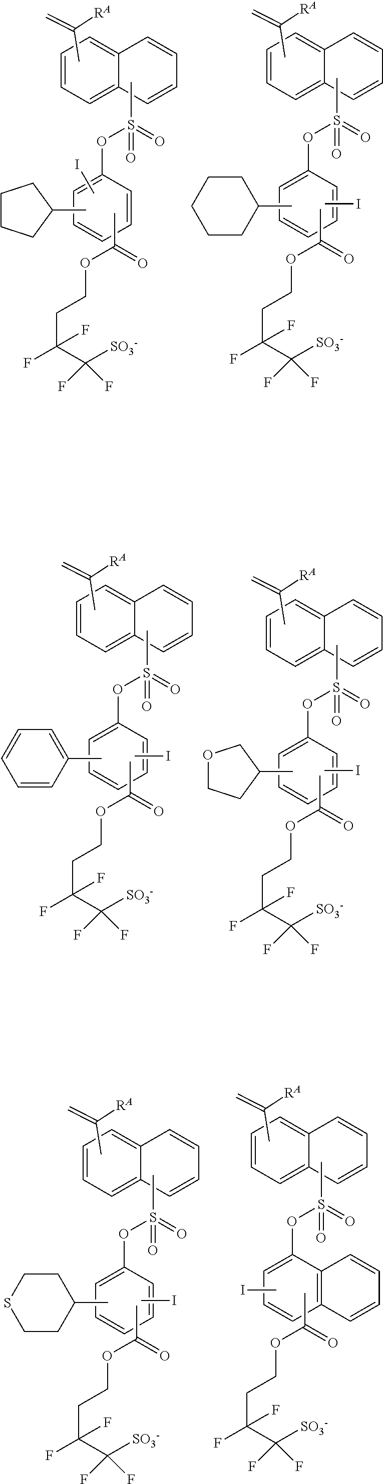

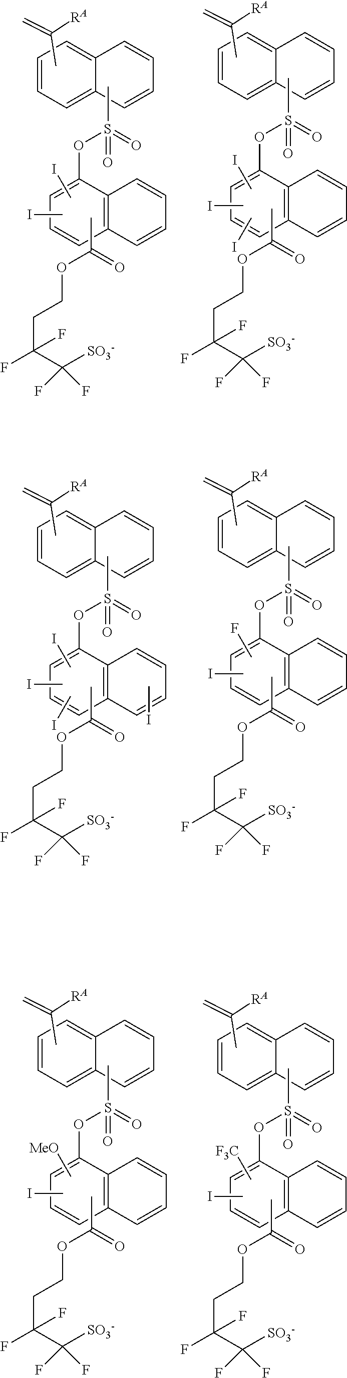

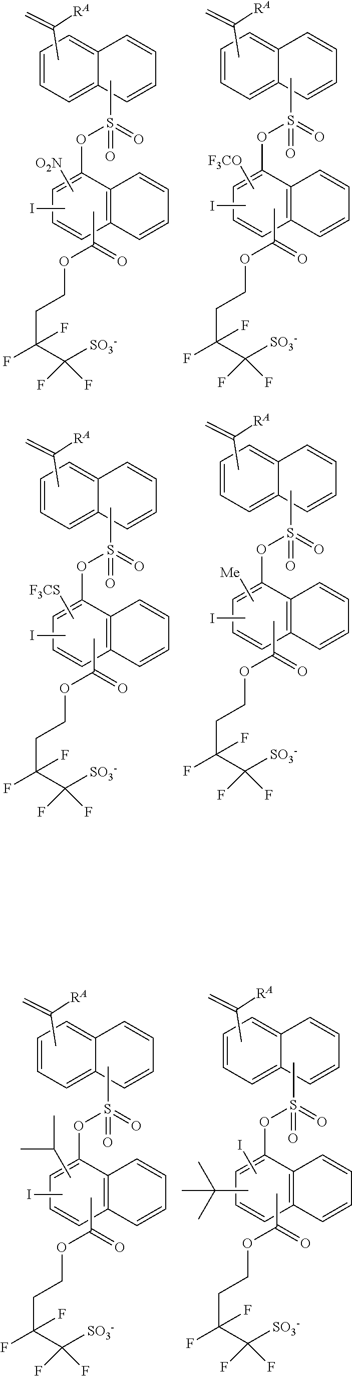

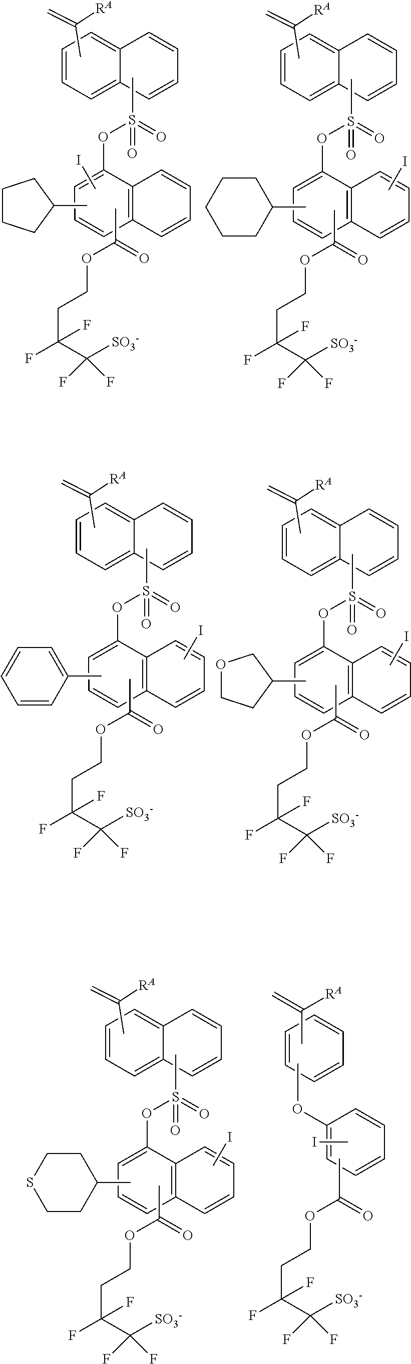

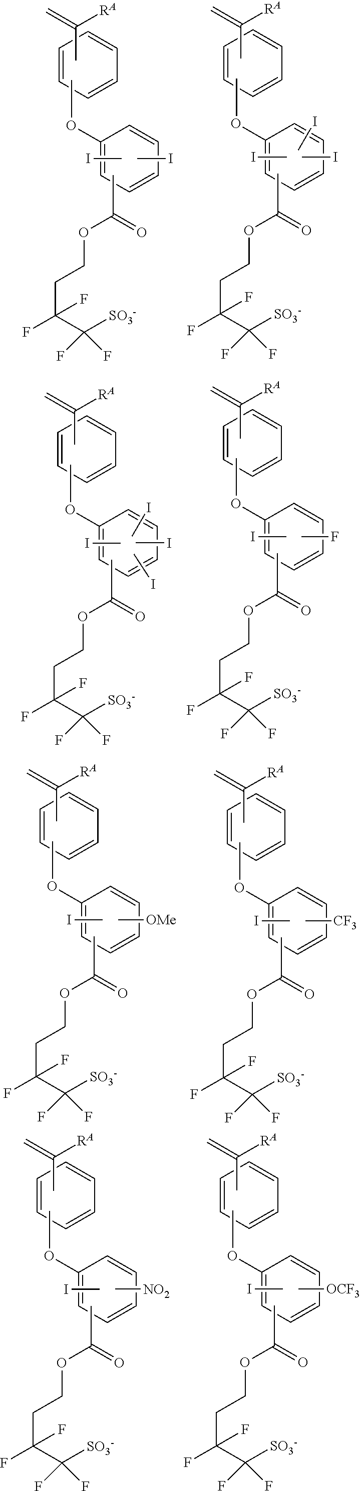

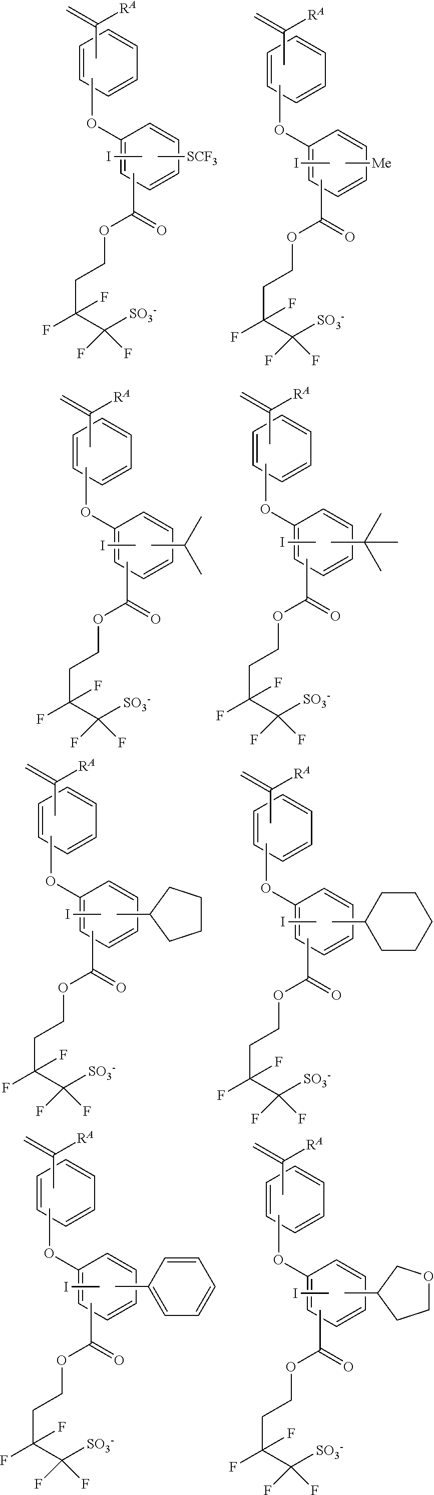

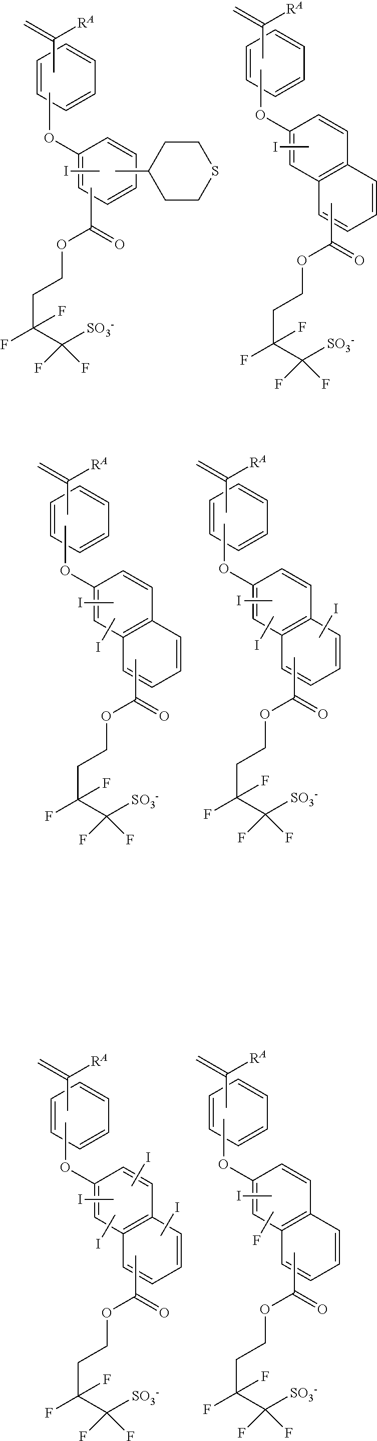

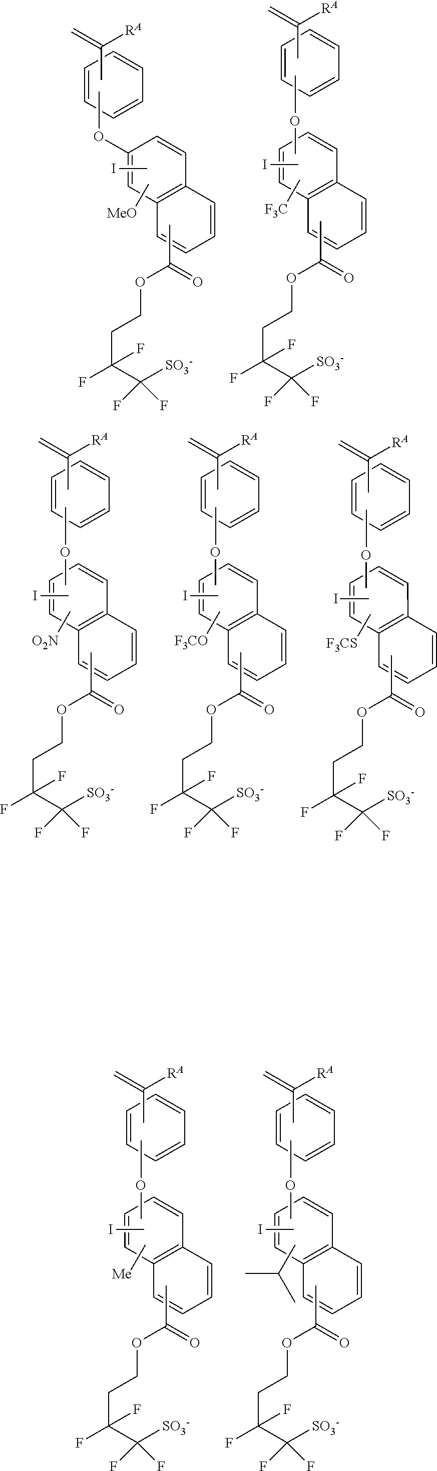

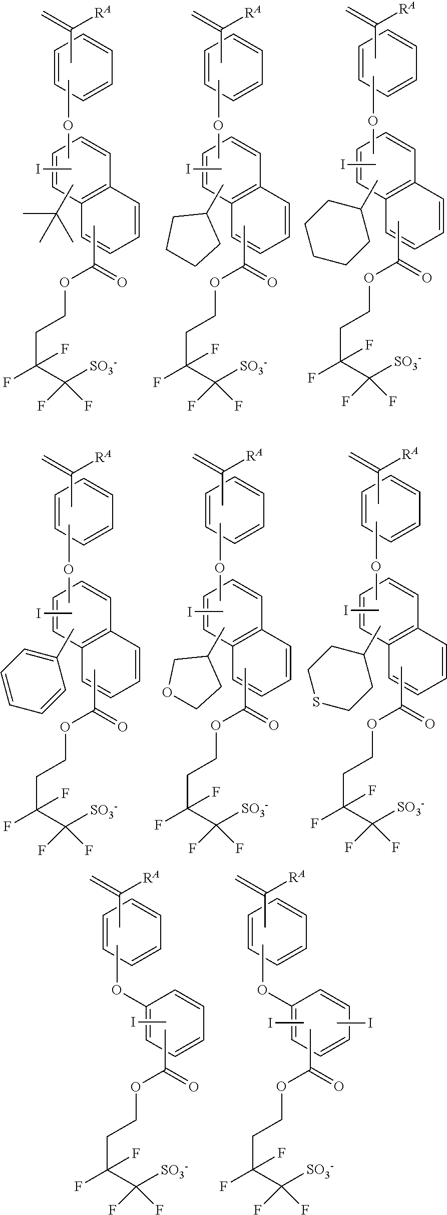

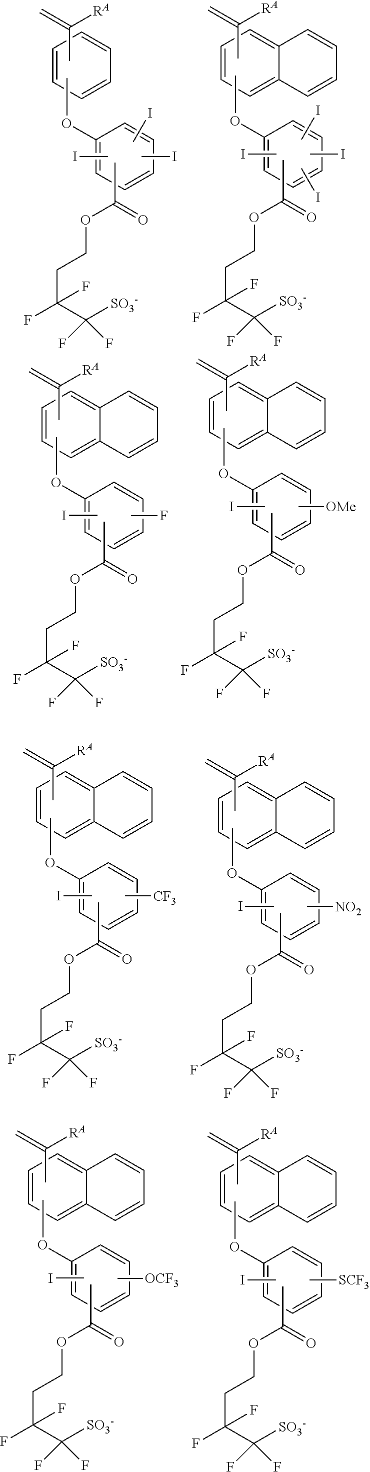

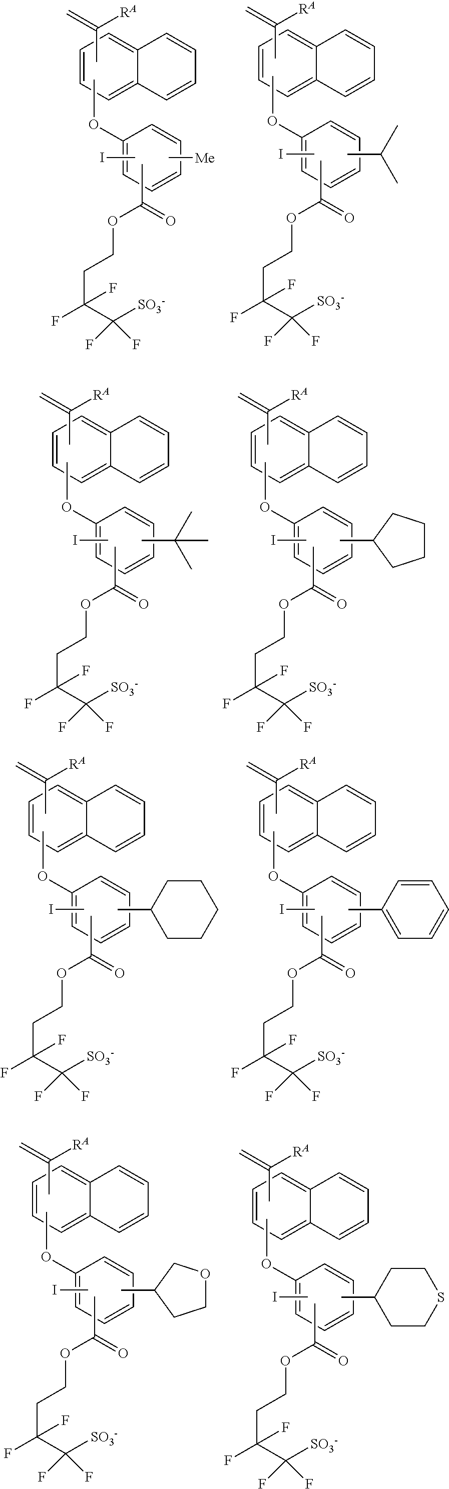

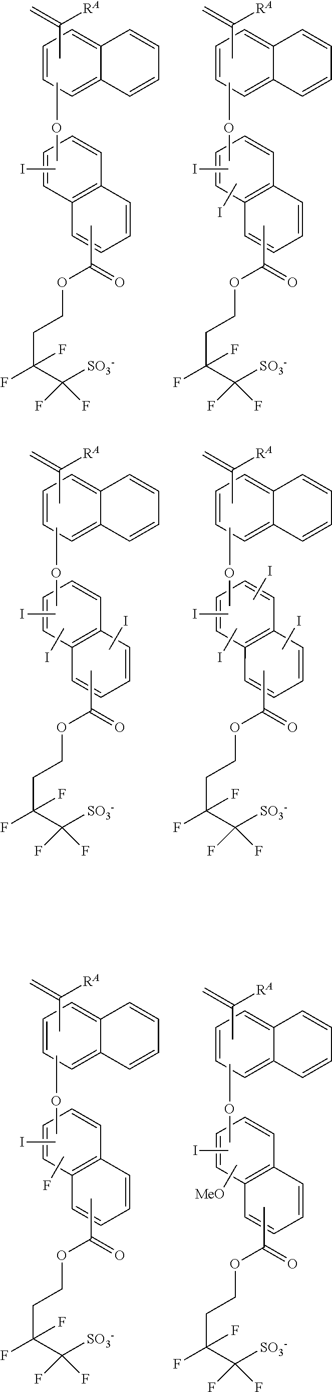

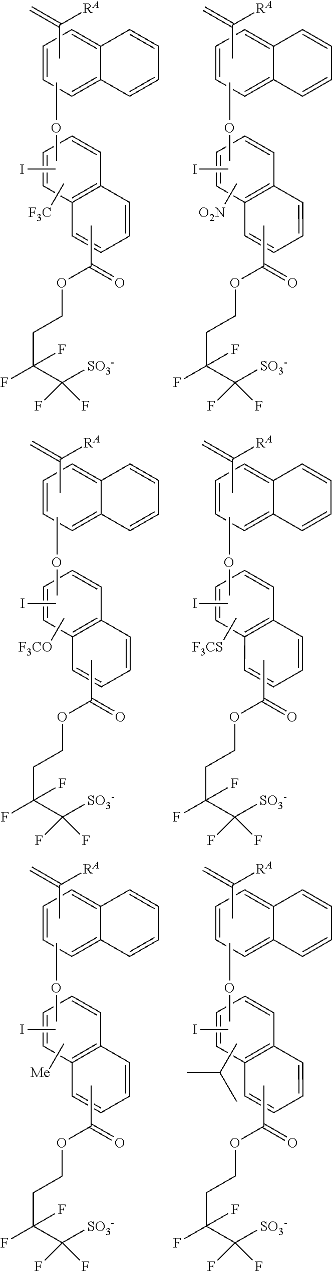

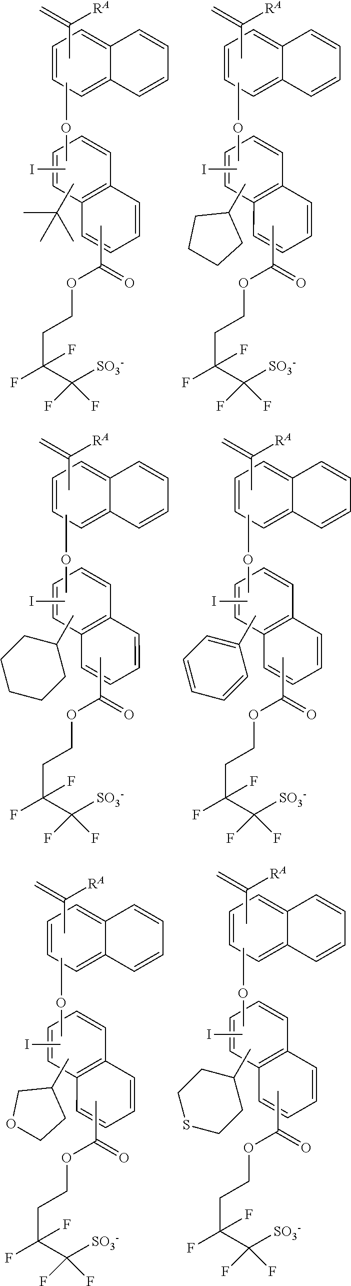

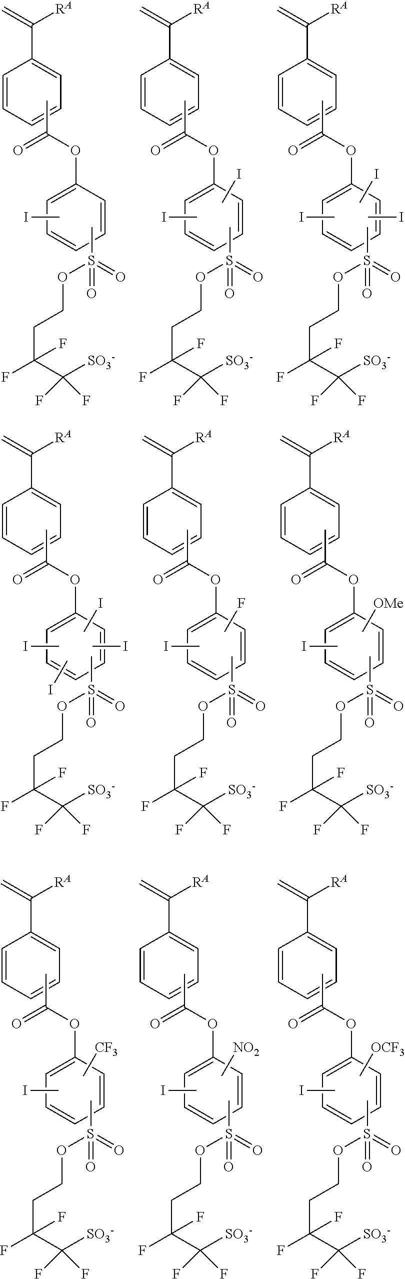

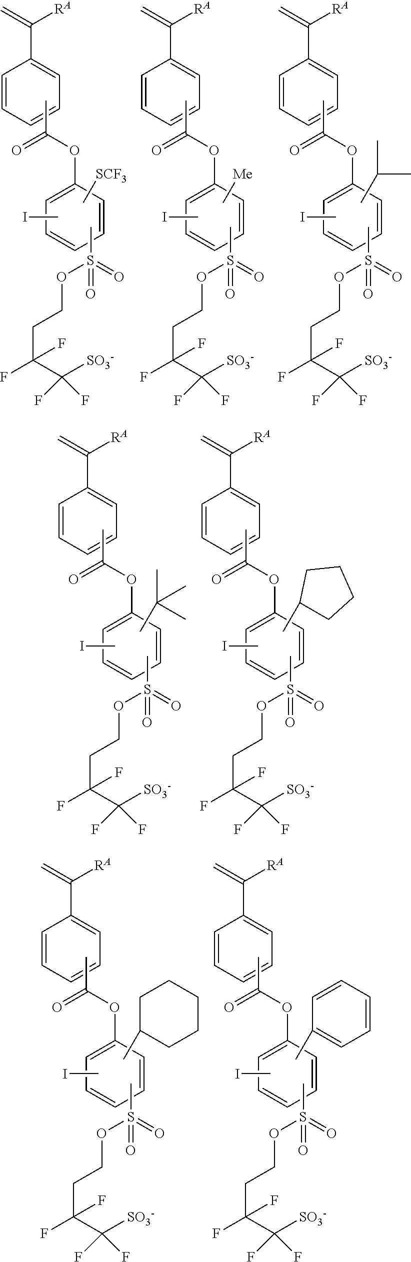

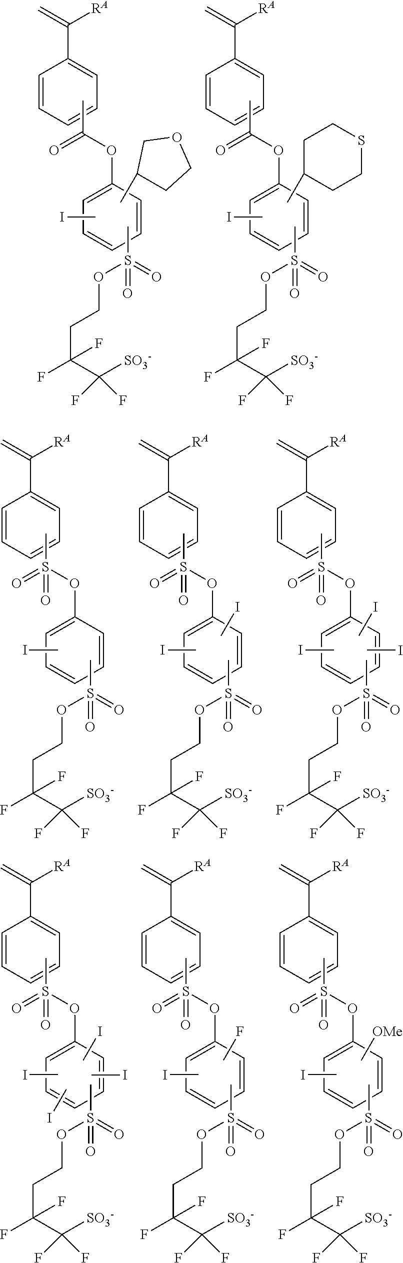

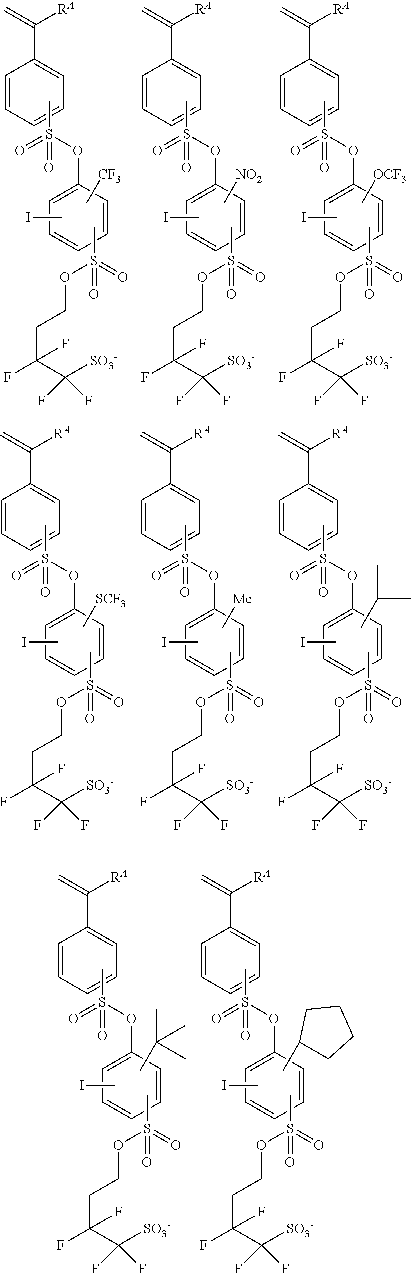

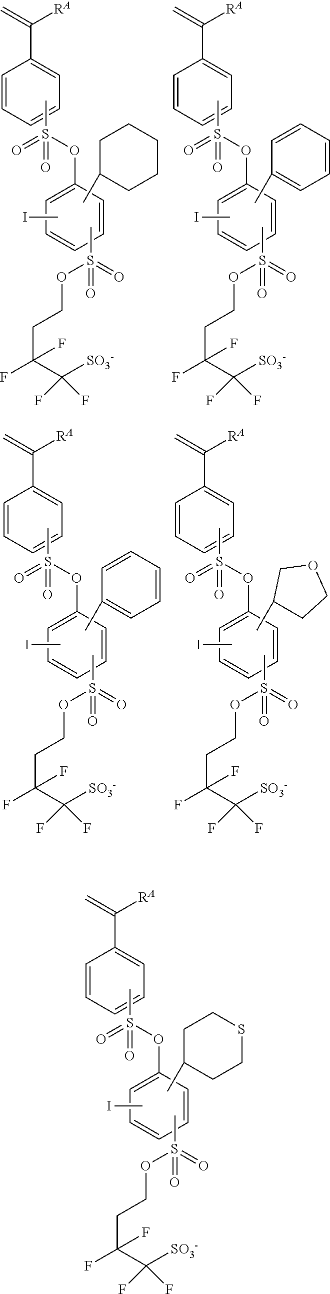

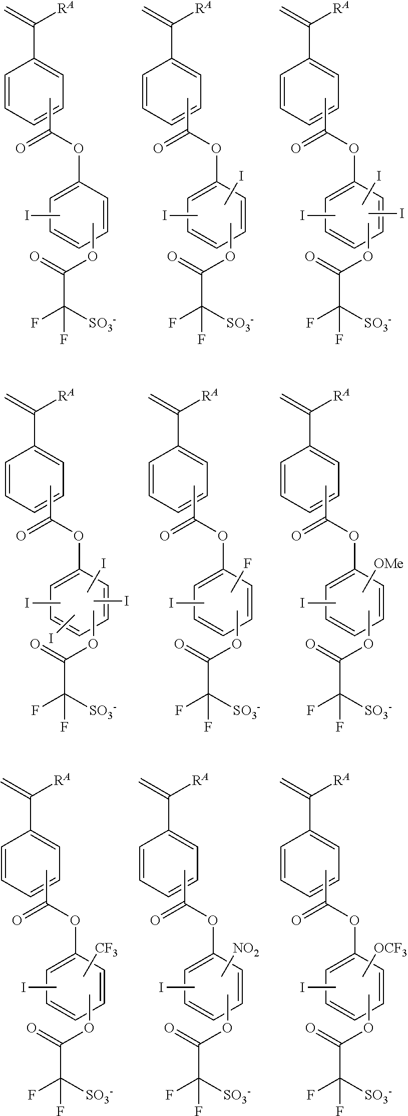

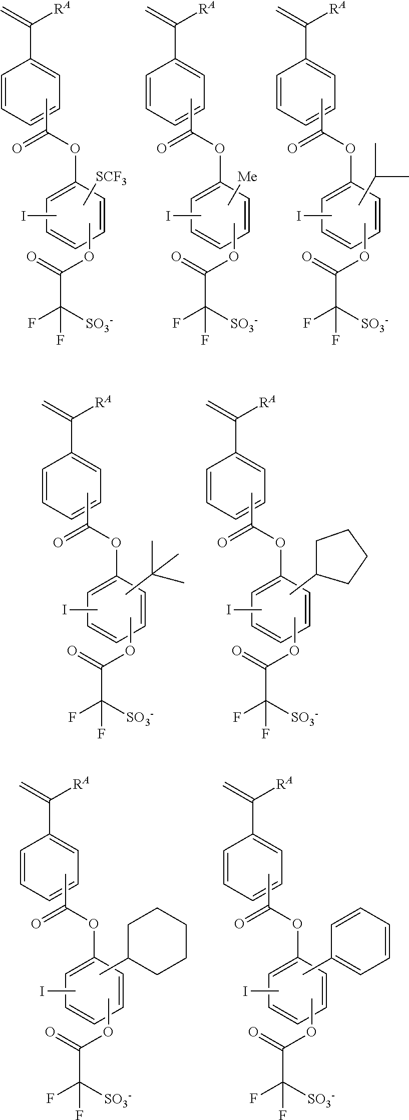

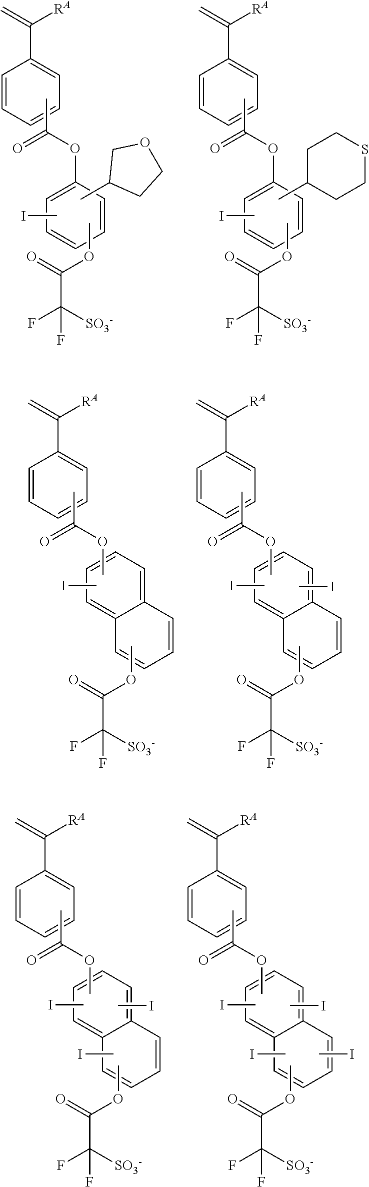

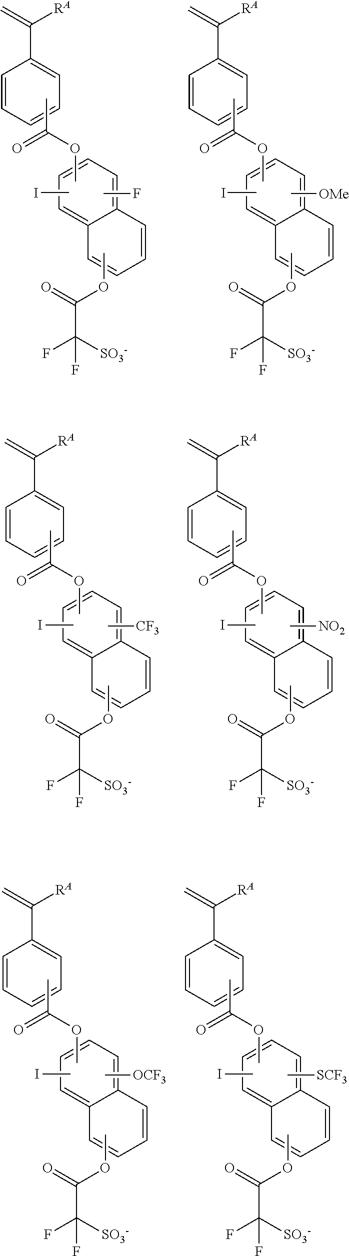

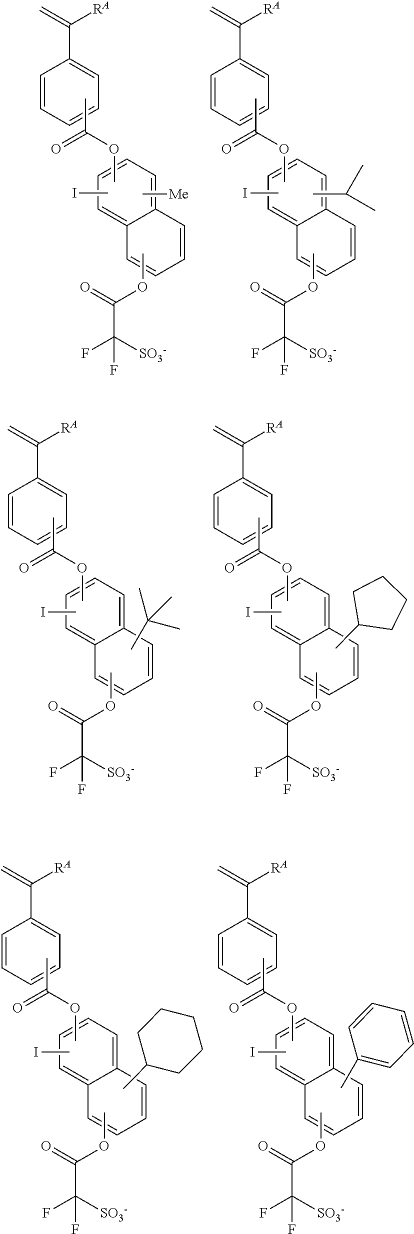

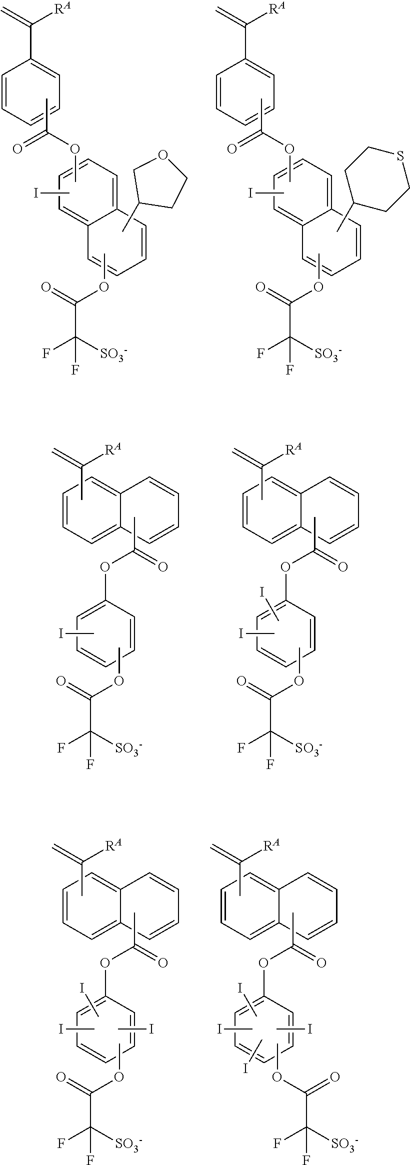

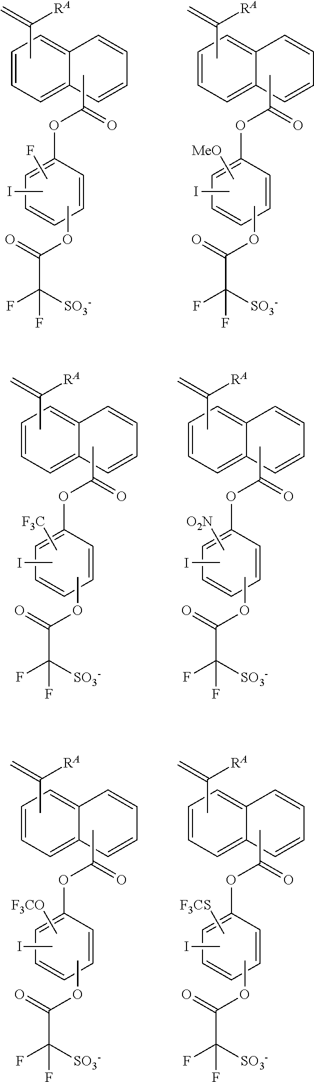

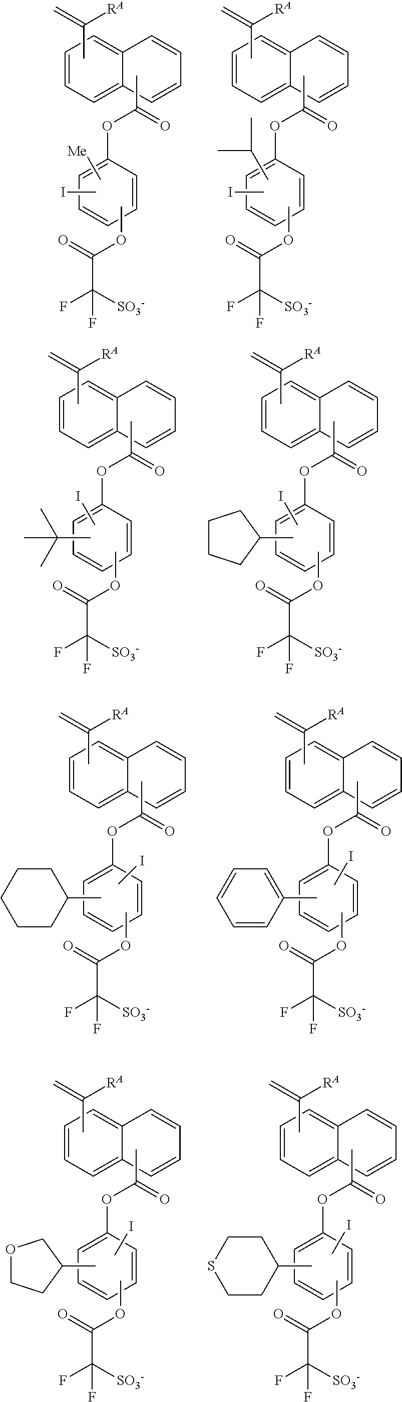

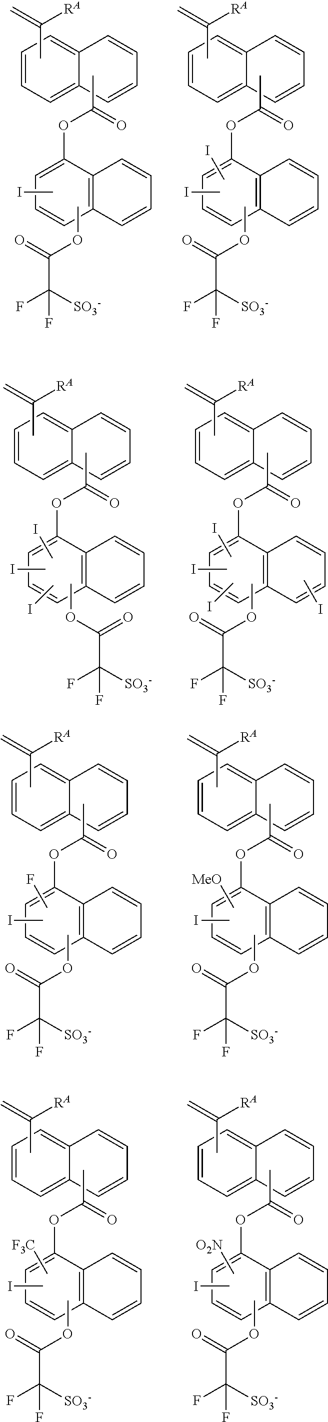

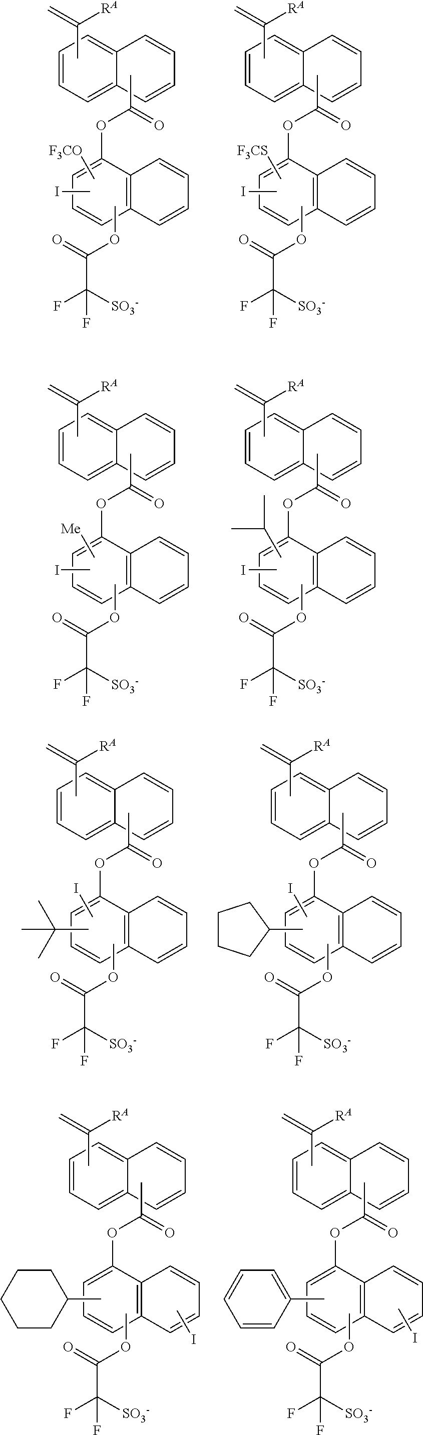

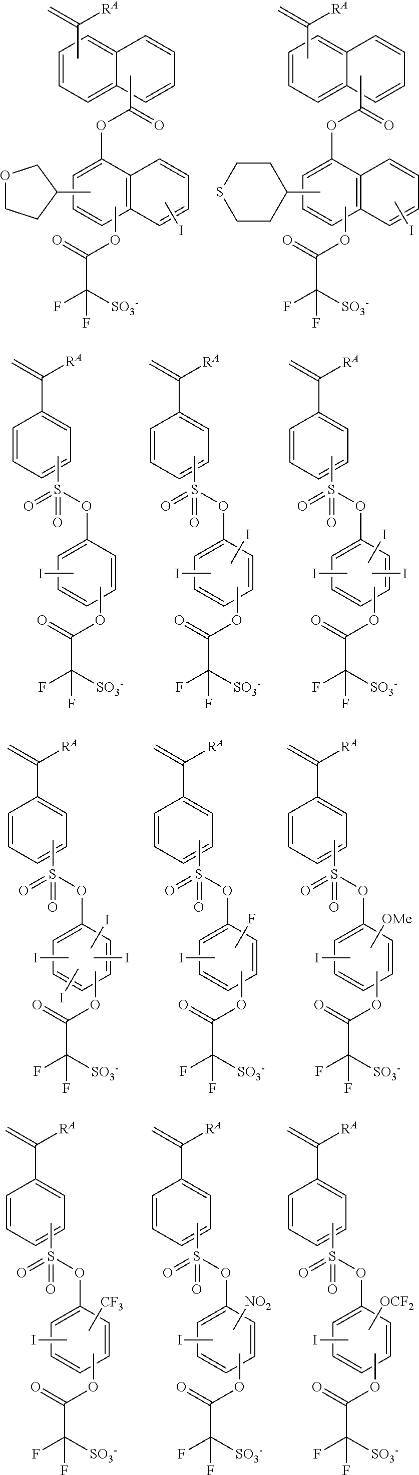

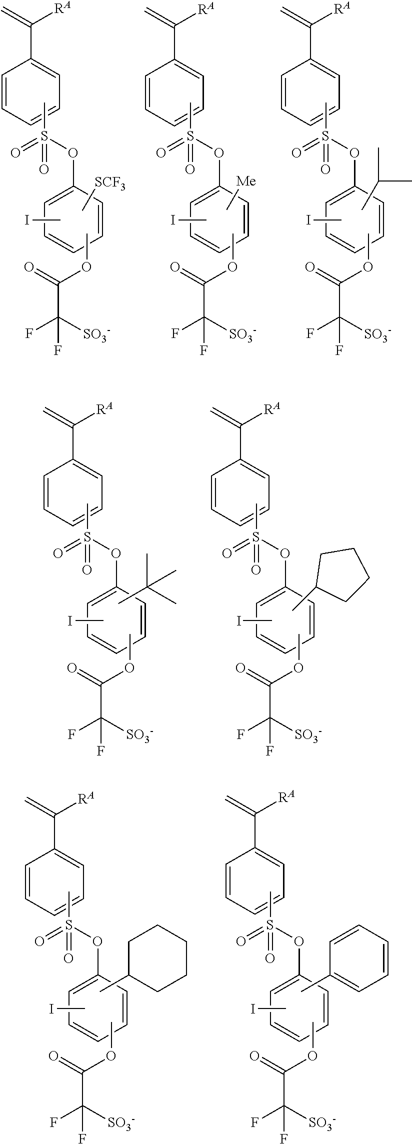

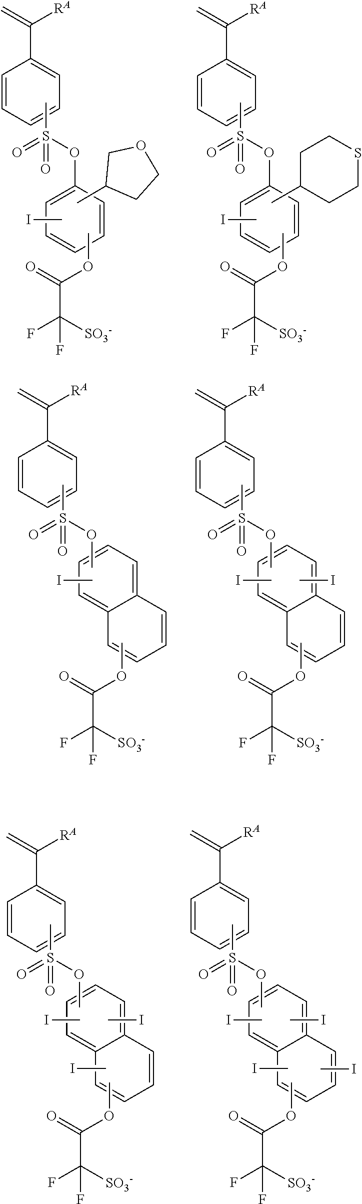

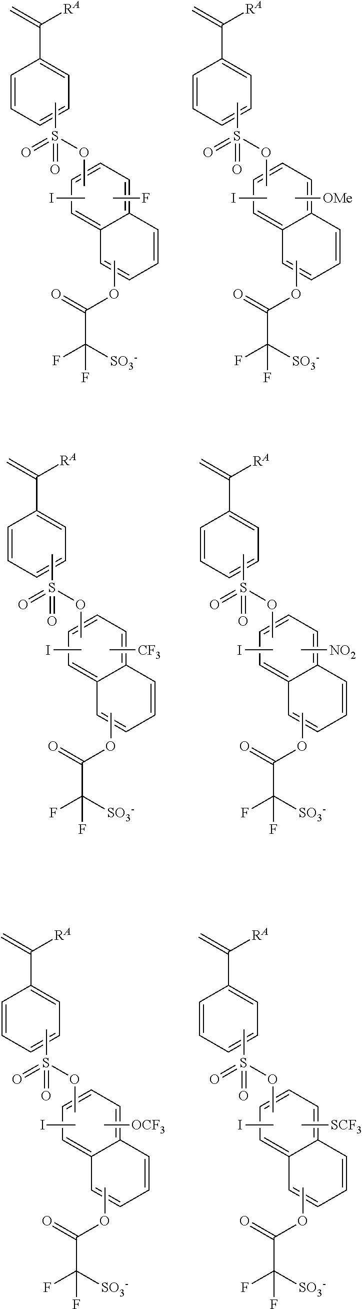

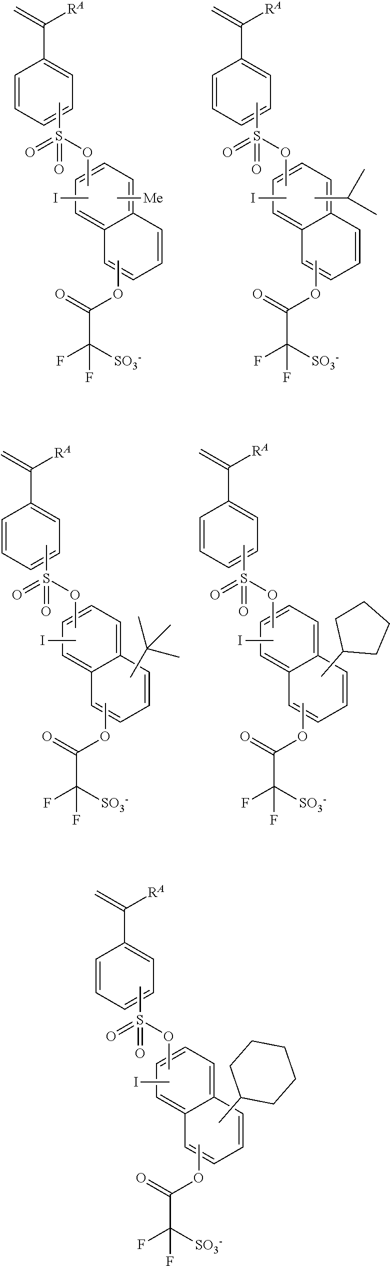

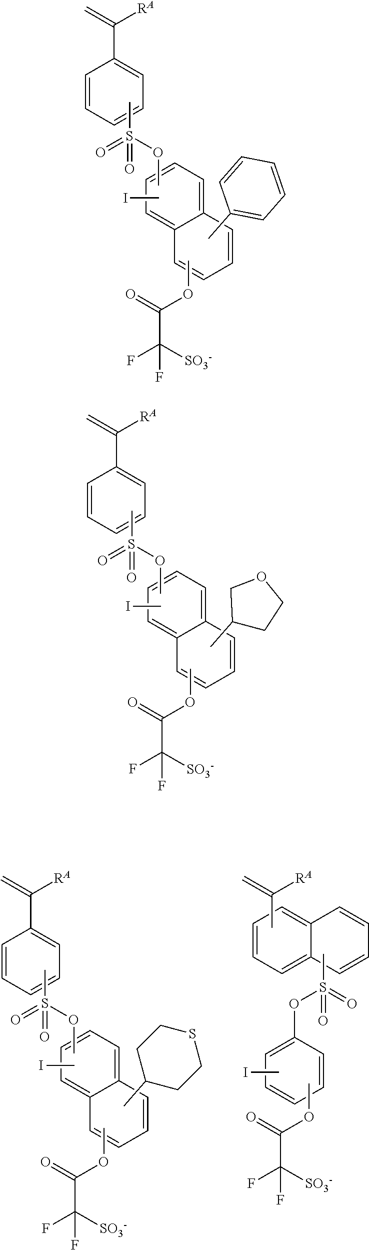

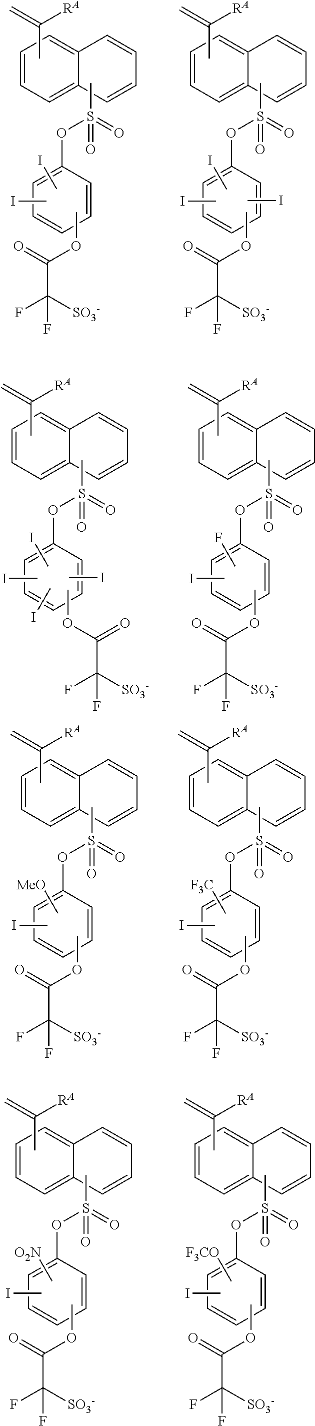

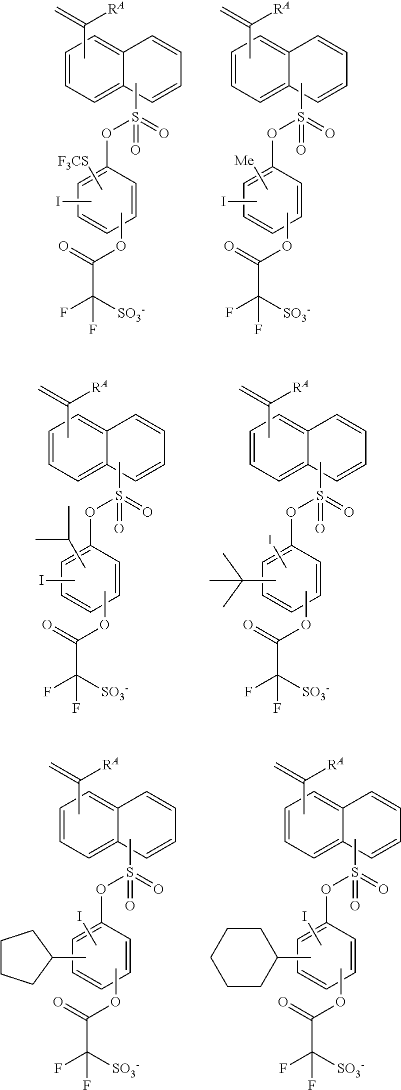

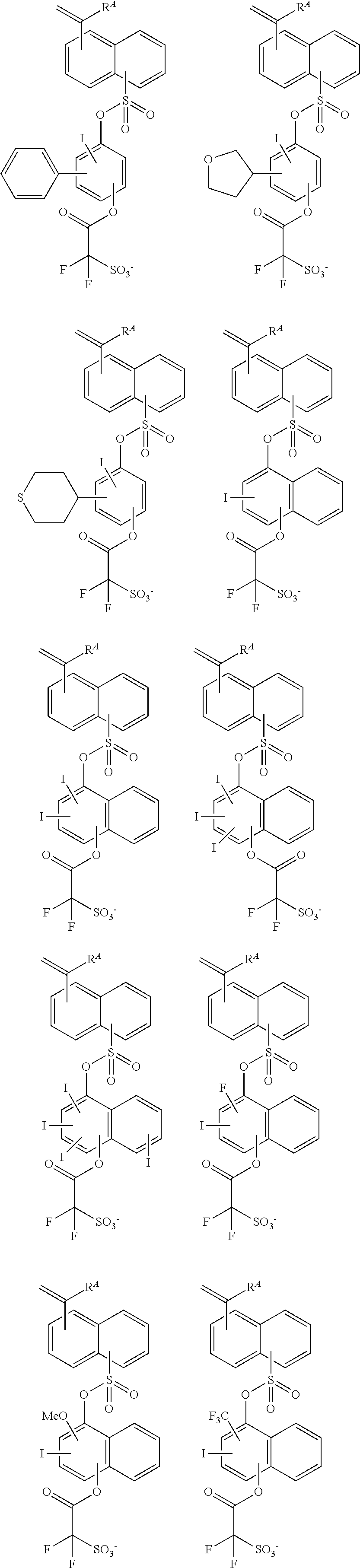

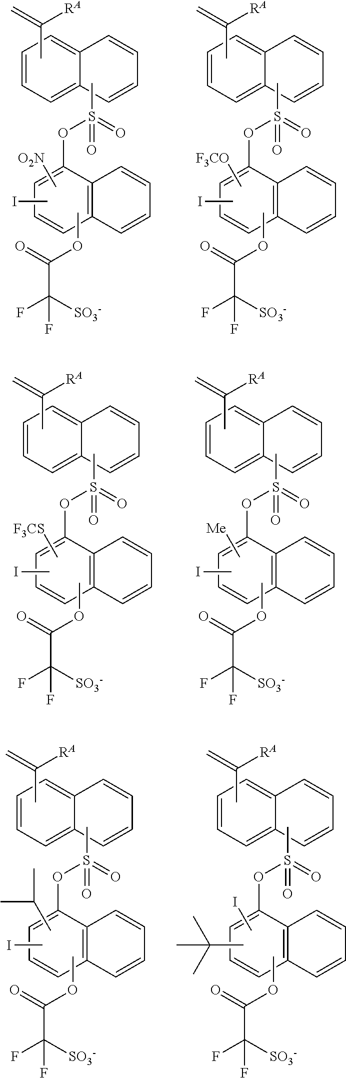

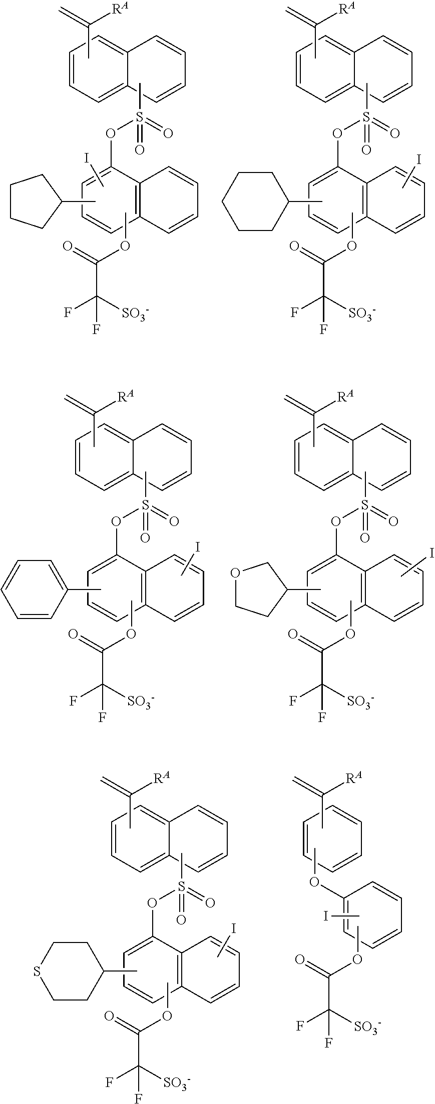

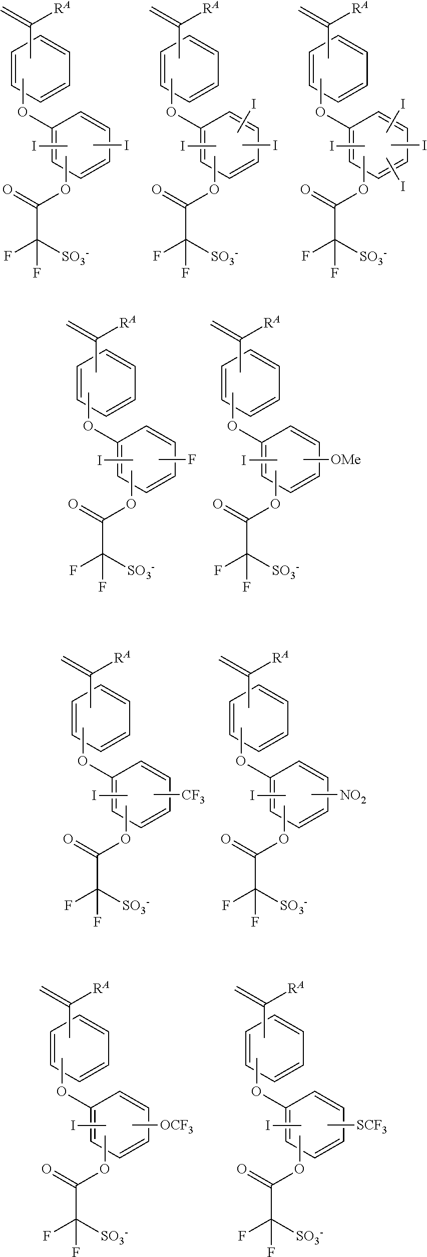

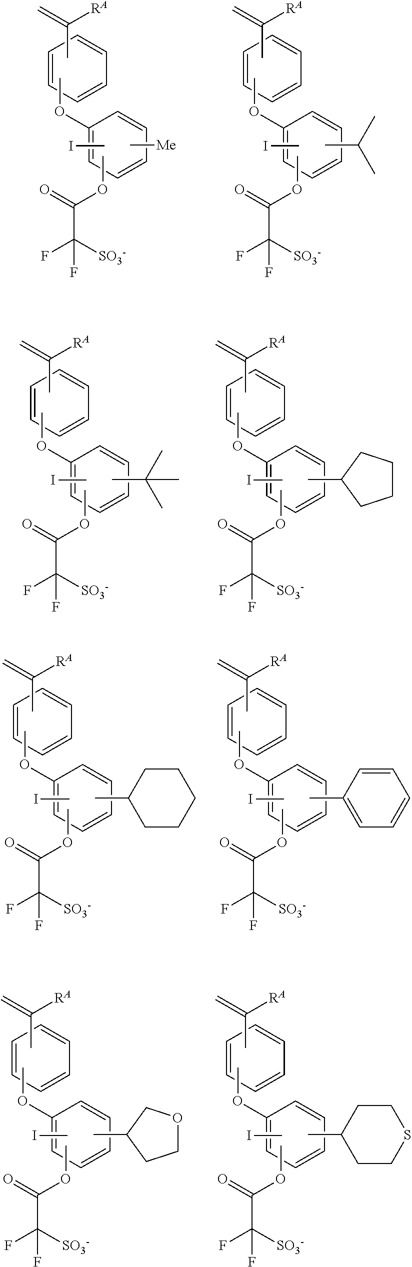

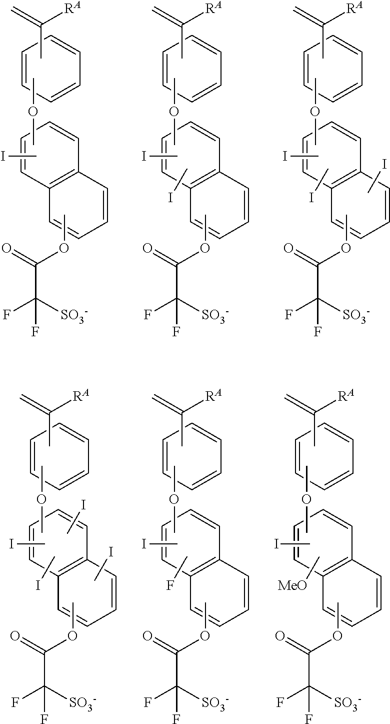

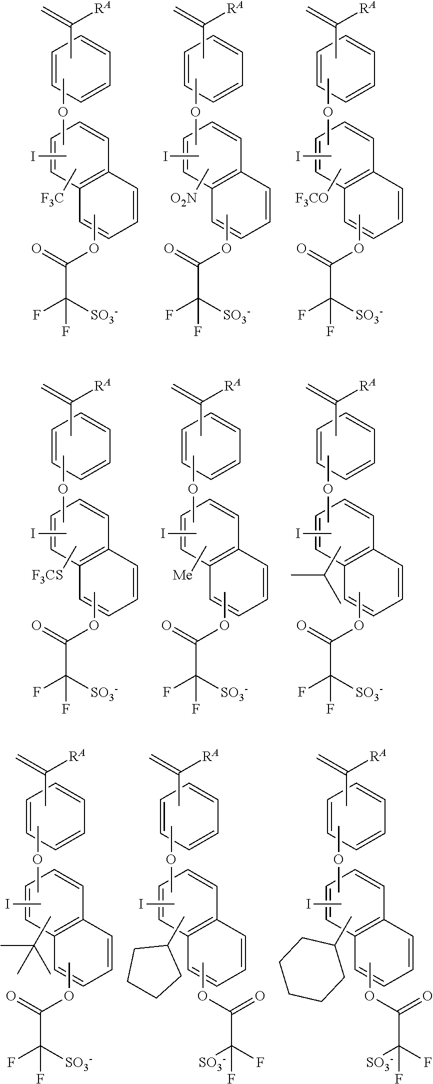

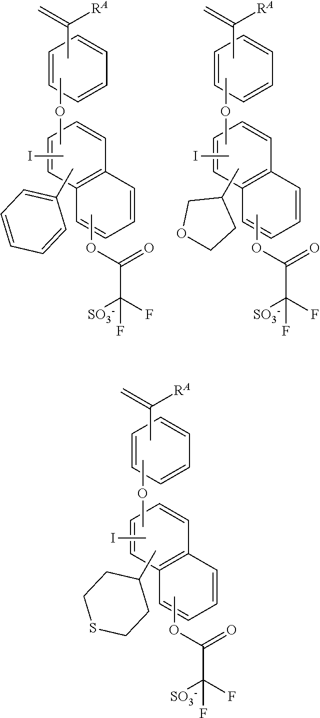

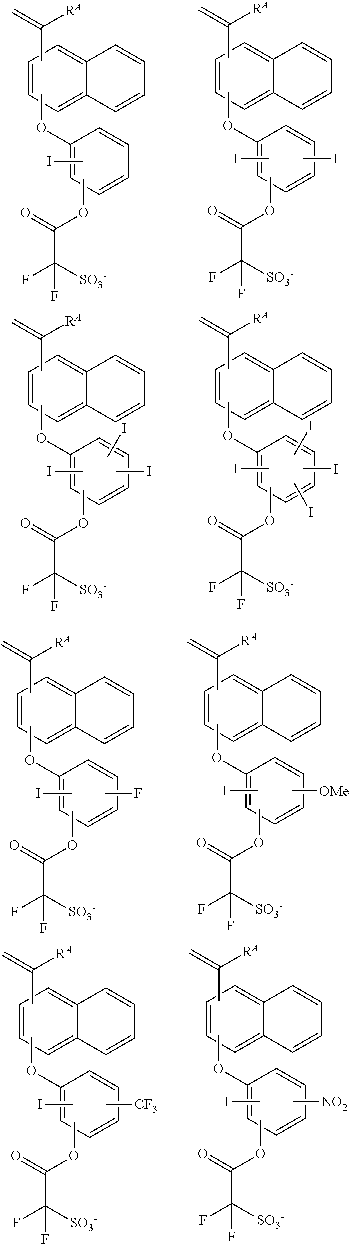

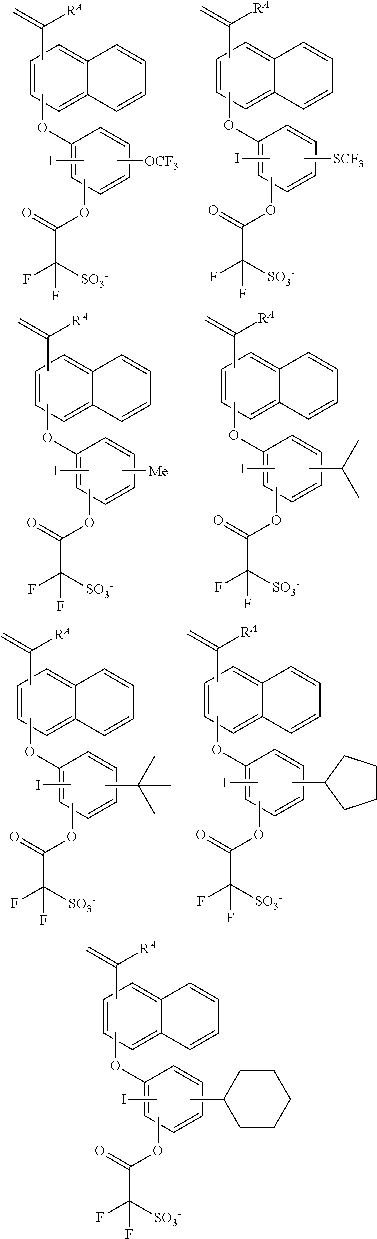

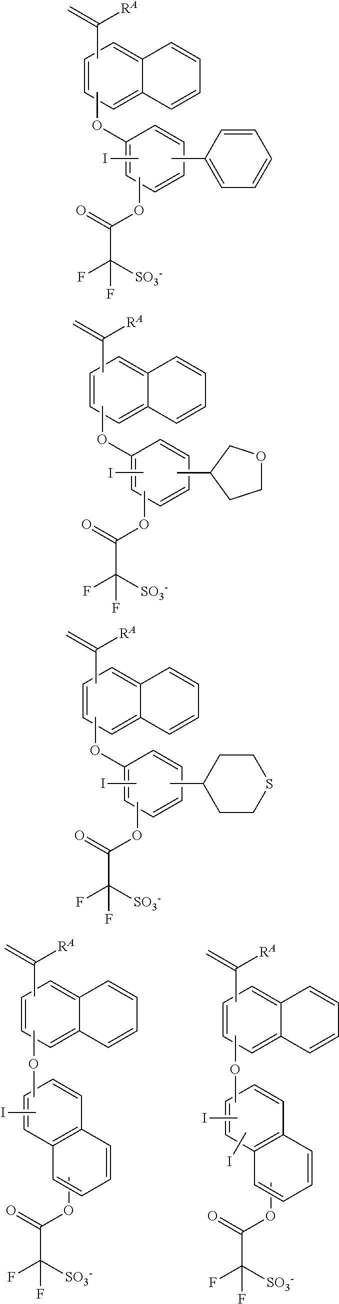

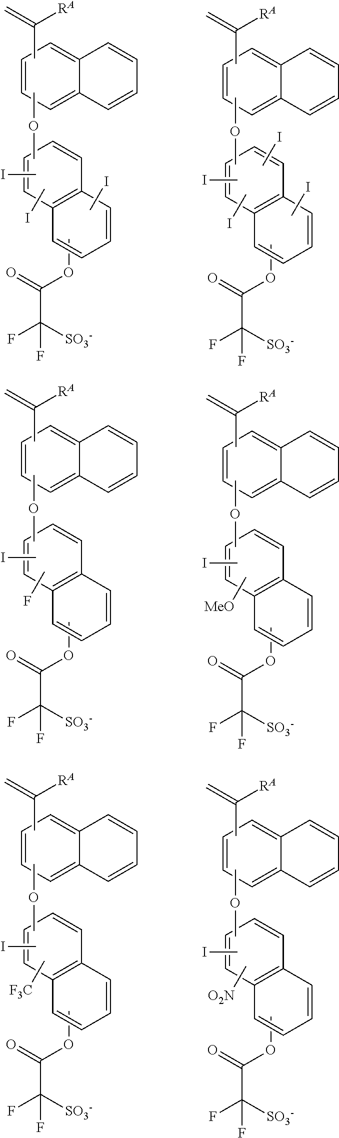

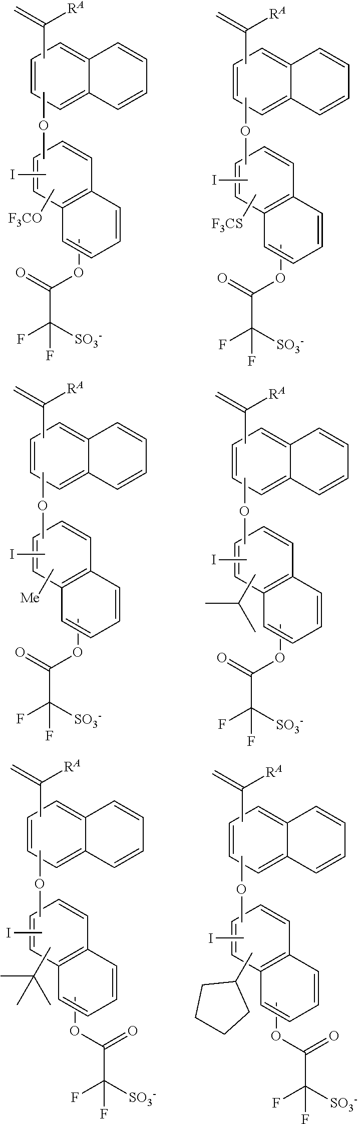

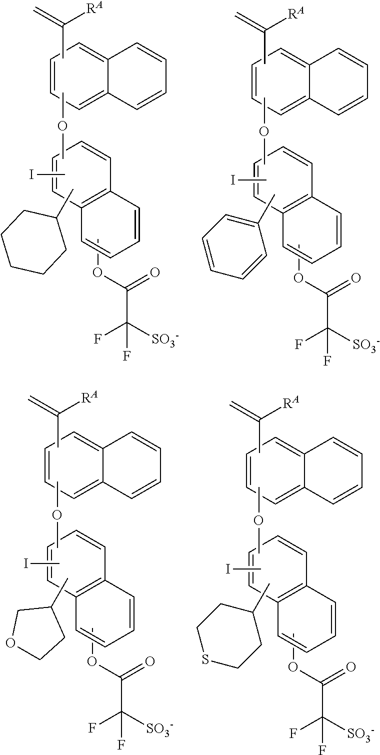

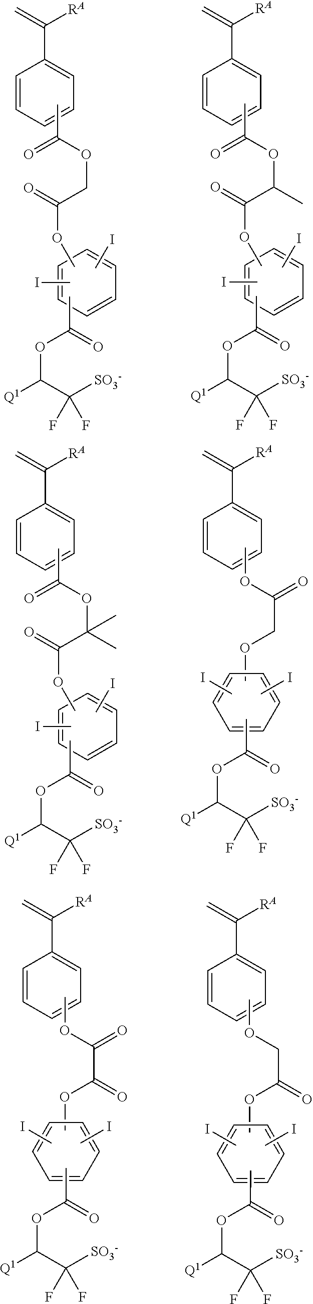

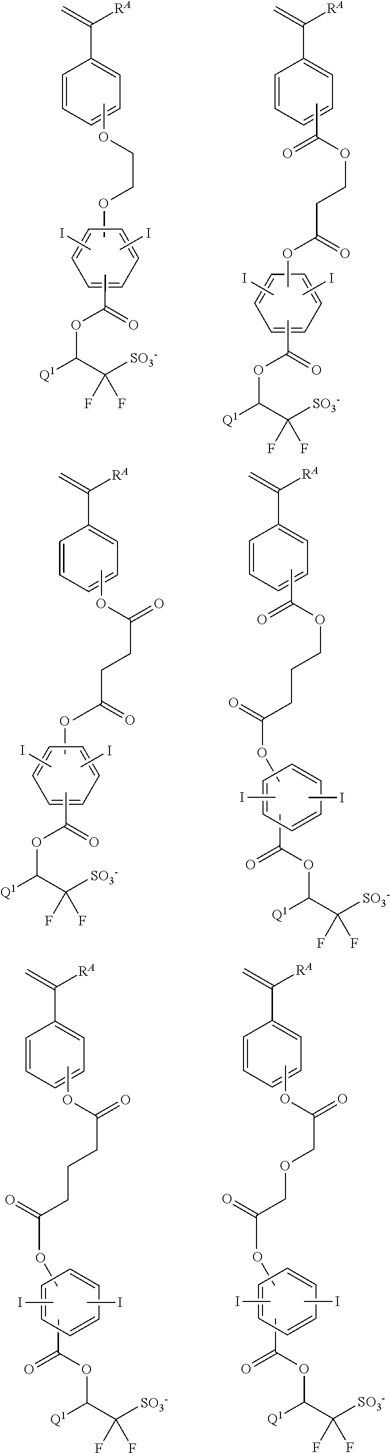

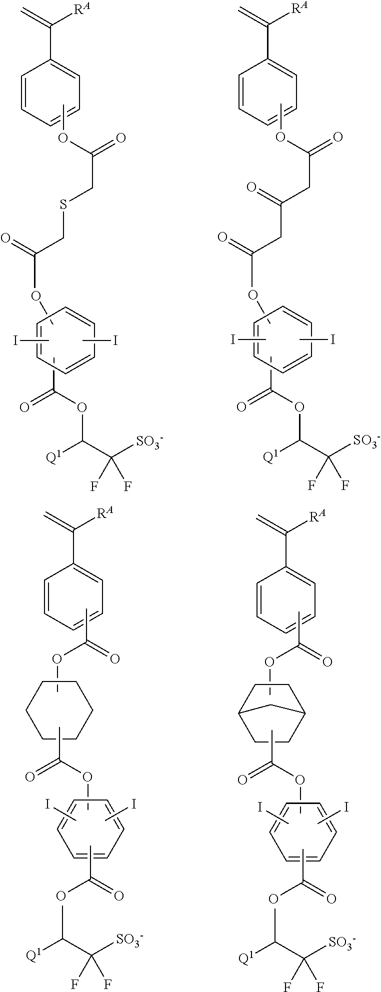

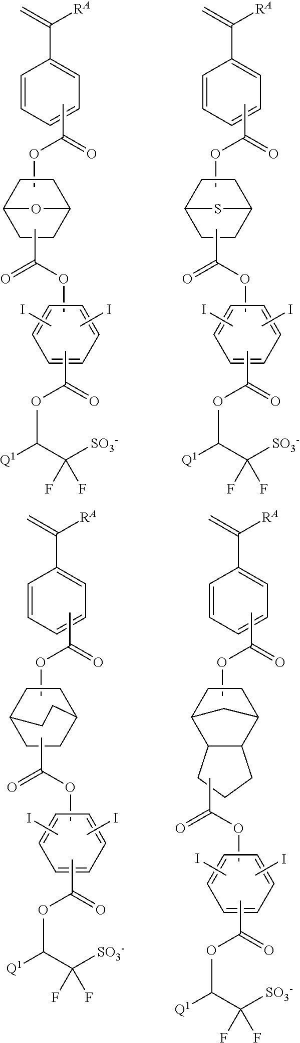

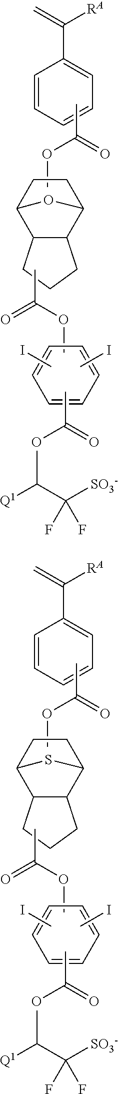

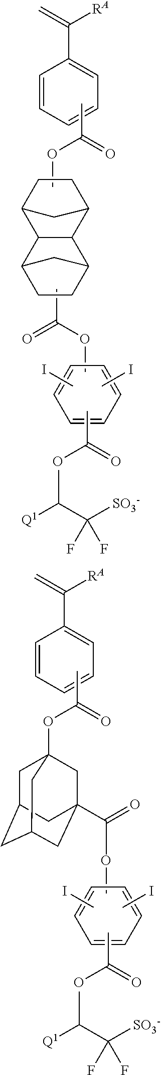

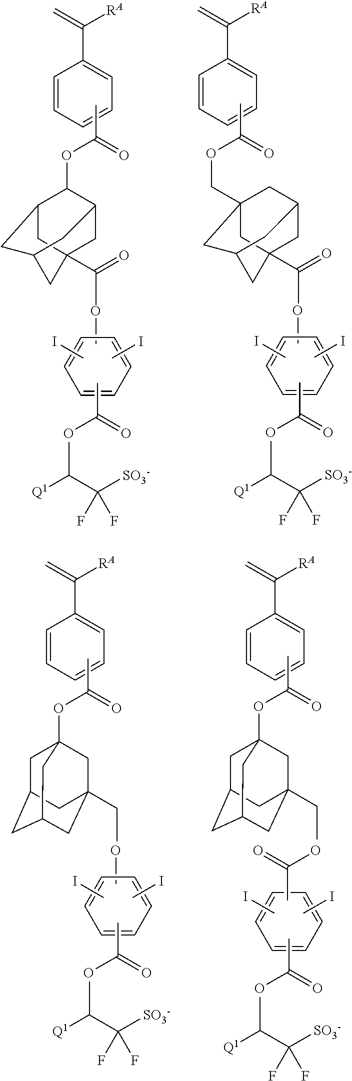

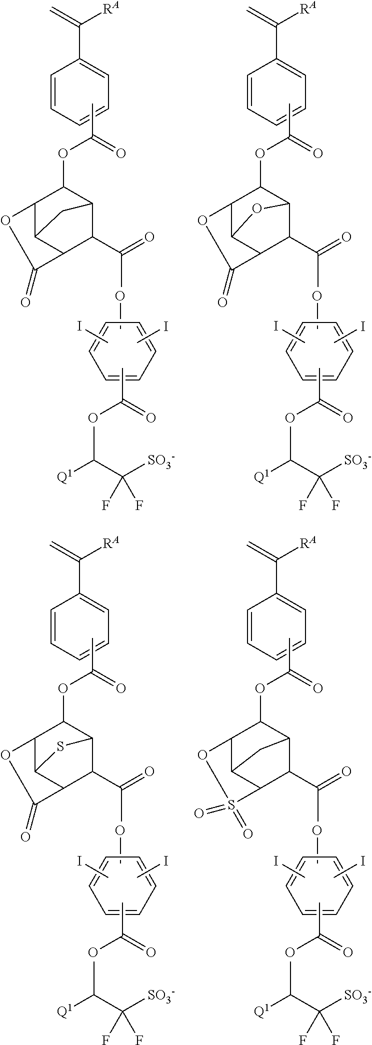

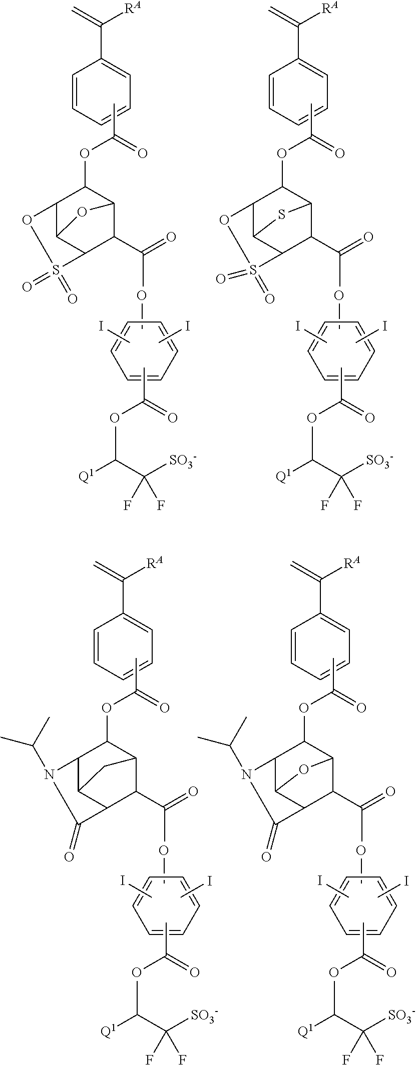

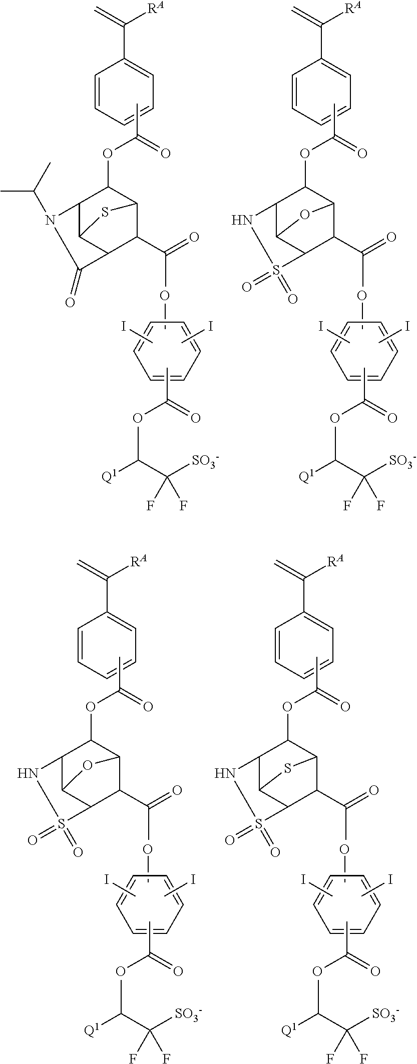

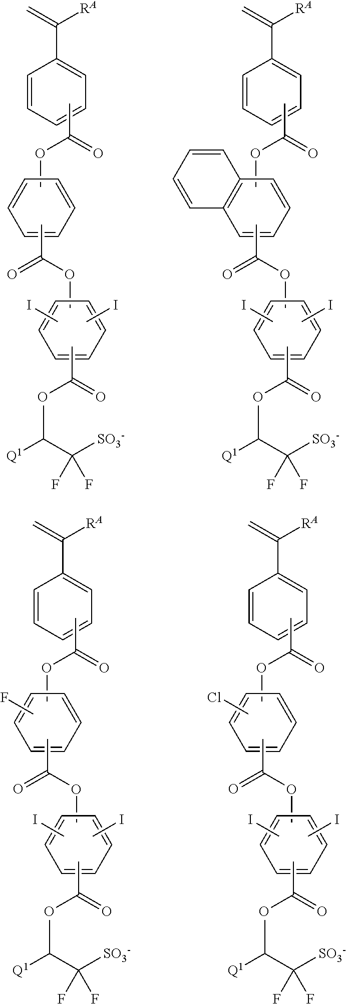

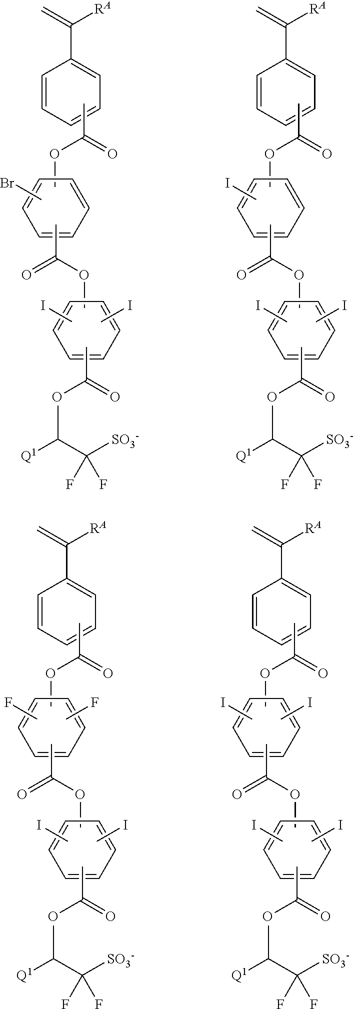

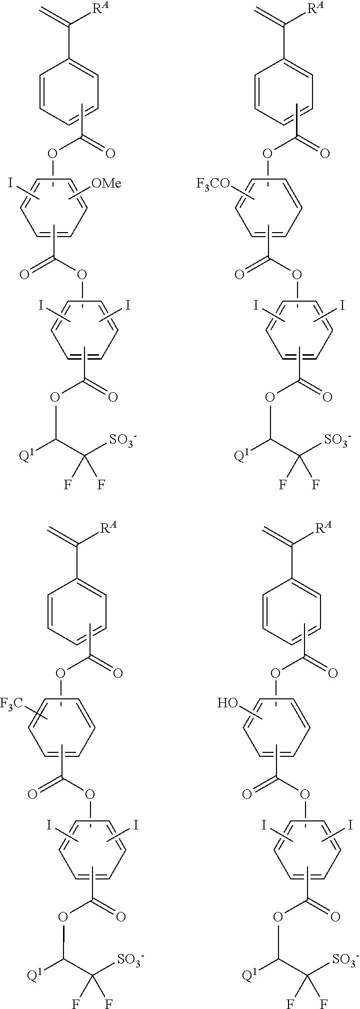

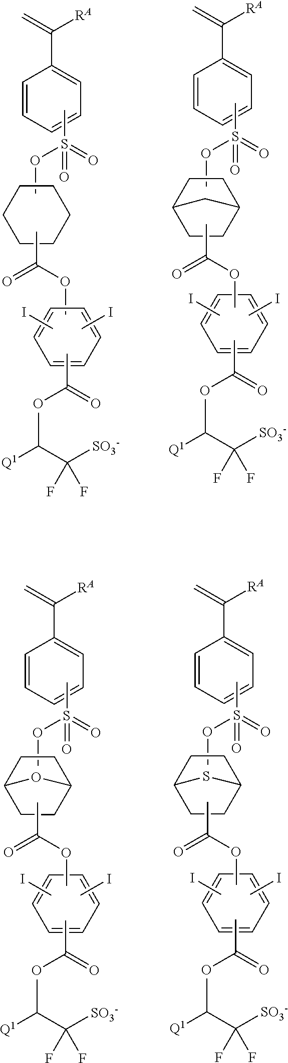

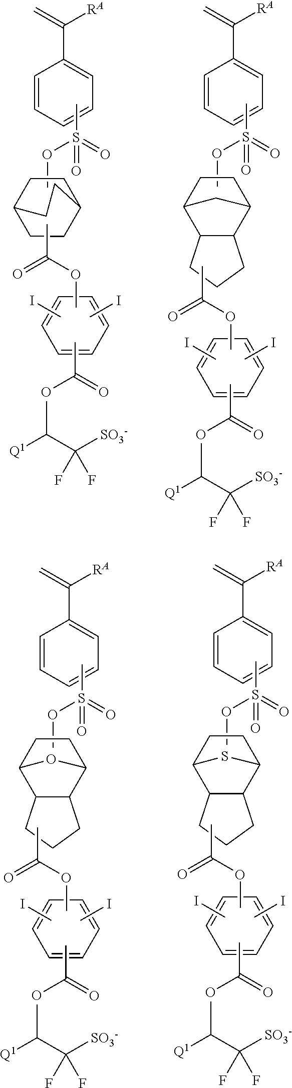

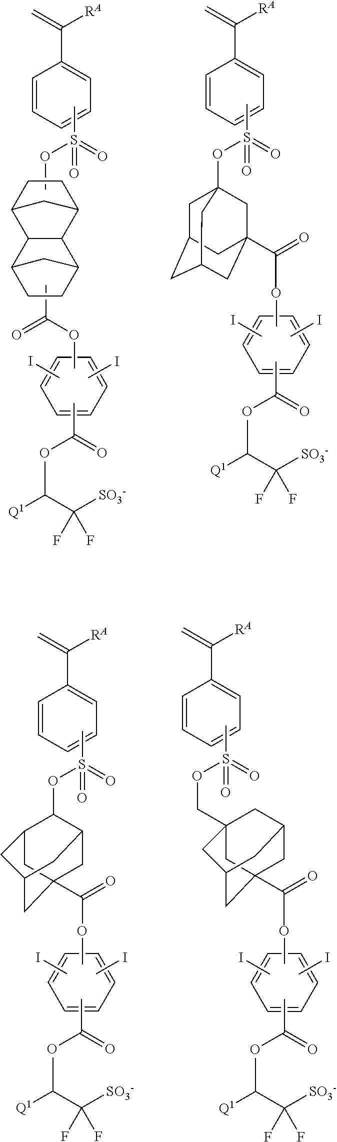

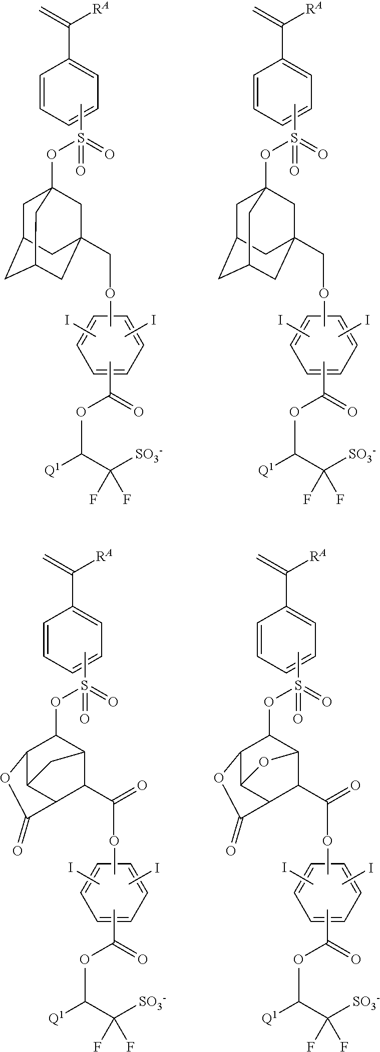

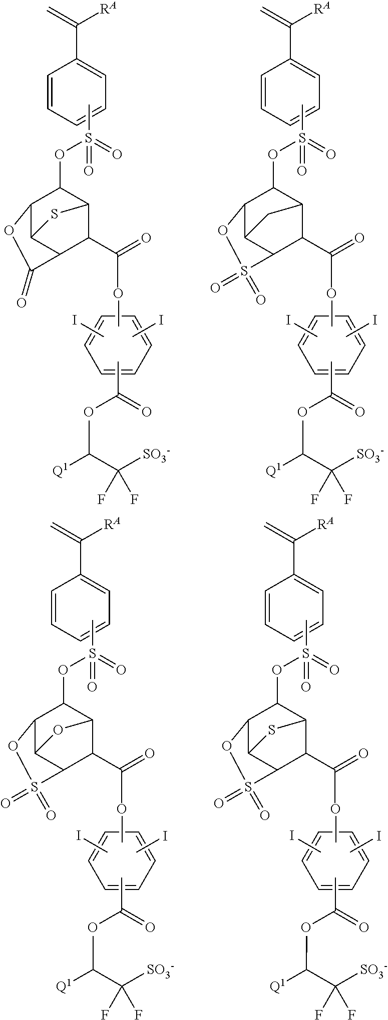

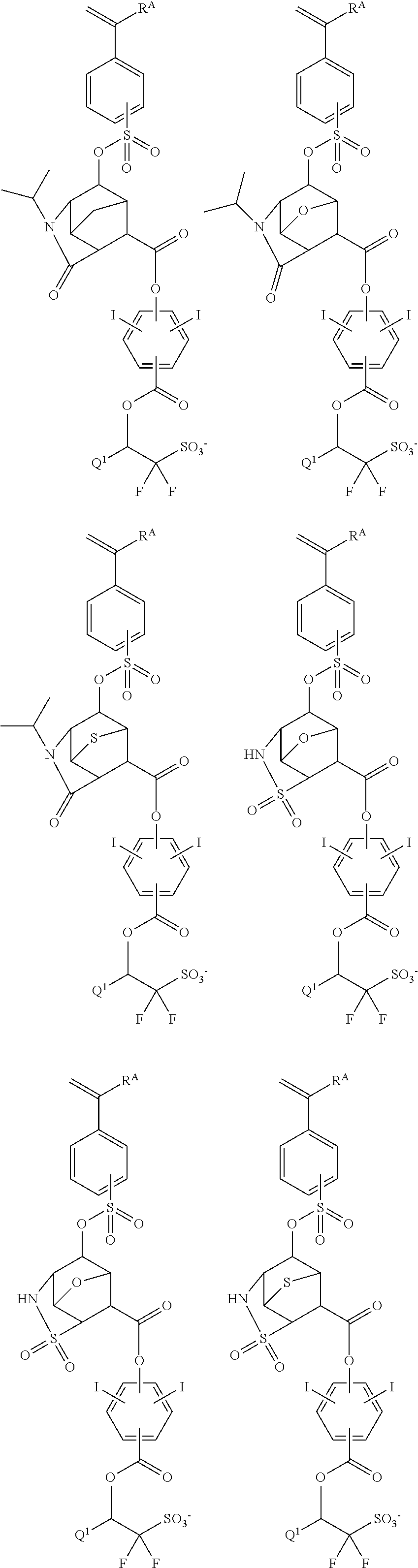

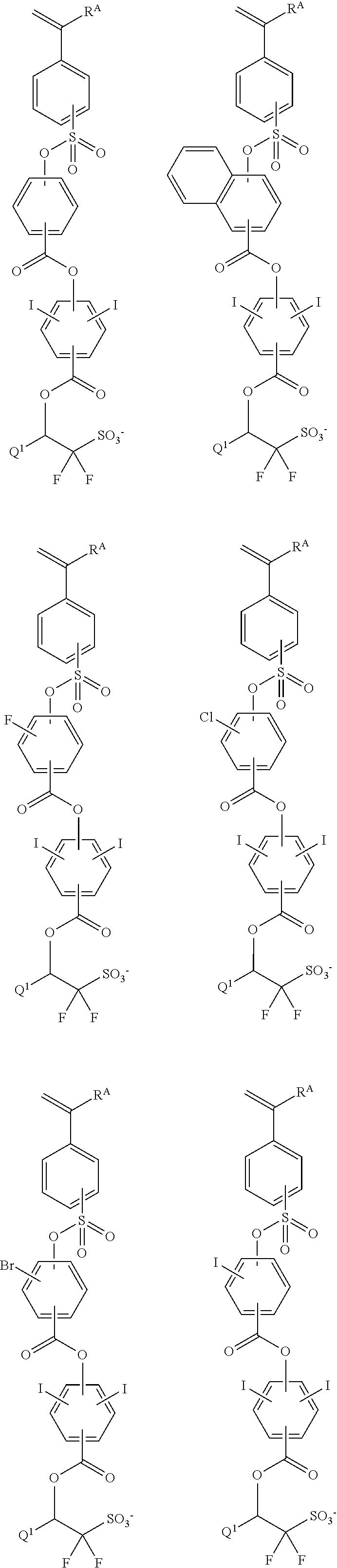

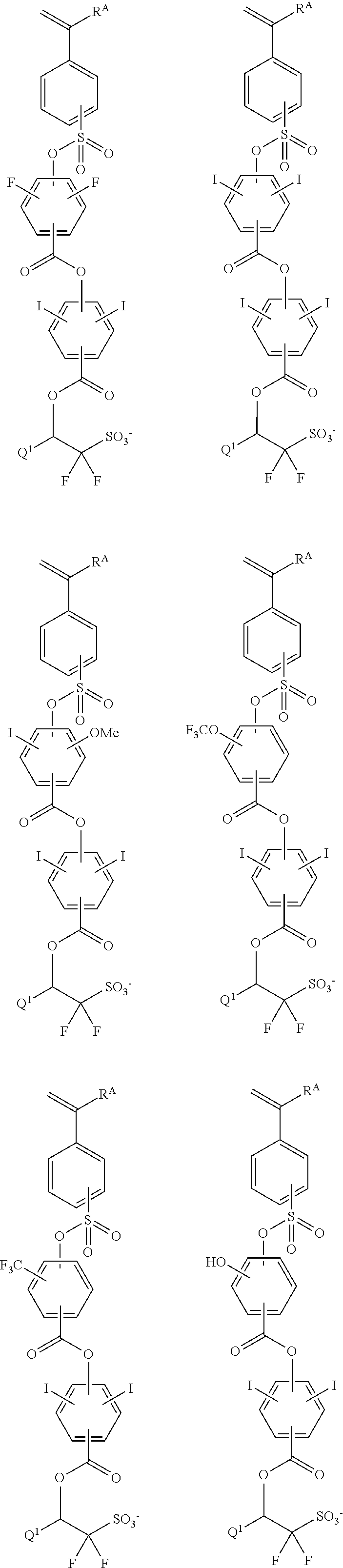

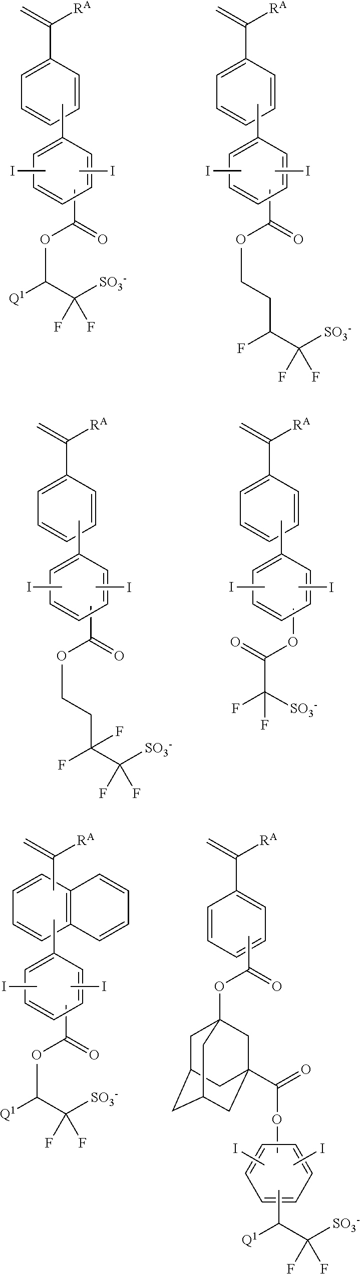

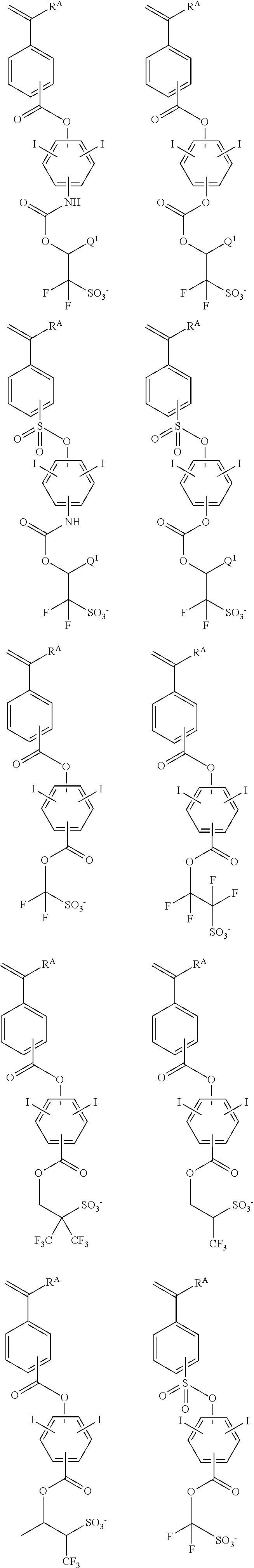

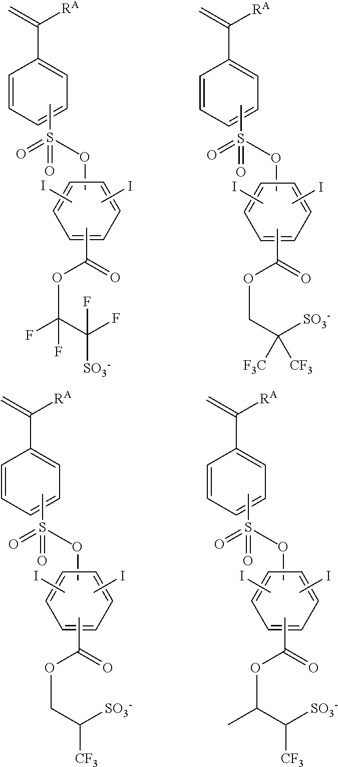

Preferable examples of the anion of the repeat unit d include an anion having the following formula (d2).

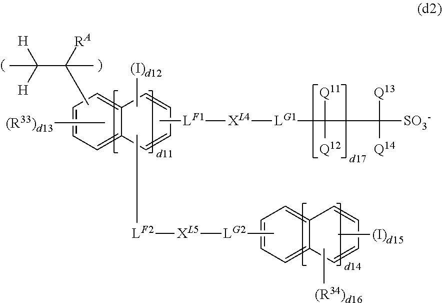

In the formula (d2), d11 is 0 or 1. When d11 is 0, it is a benzene ring, and when d11 is 1, it is a naphthalene ring, but a benzene ring in which d11 is 0 is preferable from a viewpoint of solvent solubility. d12 is 1, 2, 3, or 4. d12 is preferably 1, 2 or 3, more preferably 1 or 2, and still more preferably 1 from a viewpoint of raw material procurement. d13 is 0, 1, or 2. Provided that 1≤d12+d13≤4 when d11 is 0, and 1≤d12+d13≤6 when d11 is 1.

In the formula (d2), d14 is 0 or 1. When d14 is 0, it is a benzene ring, and when d14 is 1, it is a naphthalene ring, but a benzene ring in which d14 is 0 is preferable from a viewpoint of solvent solubility. d15 is 1, 2, 3, or 4, and preferably 1, 2, or 3. The larger the number of iodine atoms in the anion structure, the higher the absorption particularly for EUV, but since there is a concern that solvent solubility is poor and precipitation occurs in a resist composition, the number of iodine atoms in the anion is preferably 2, 3, 4, or 5, and more preferably 2, 3, or 4. d16 is 0, 1, or 2. Provided that 1≤d15+d16≤4 when d14 is 0, and 1≤d15+d16≤6 when d14 is 1.

In the formula (d2), d17 is 0, 1, 2, 3, or 4, preferably 0, 1, 2, or 3, more preferably 1, 2, or 3, and still more preferably 1.

In the formula (d2), R^{A} is a hydrogen atom, a fluorine atom, a methyl group, or a trifluoromethyl group. Among these, R^{A} is preferably a hydrogen atom or a methyl group, and more preferably a hydrogen atom.

In the formula (d2), R³³ is a halogen atom other than an iodine atom, a nitro group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. The halogen atom other than an iodine atom is preferably a fluorine atom, a chlorine atom, or a bromine atom, and more preferably a fluorine atom. The hydrocarbyl group and hydrocarbyl moieties of the hydrocarbyloxy group and the hydrocarbylthio group may be saturated or unsaturated, and may be linear, branched, or cyclic. Specific examples thereof include: a C₁-C₂₀ alkyl group such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, or an icosyl group; a C₃-C₂₀ cyclic saturated hydrocarbyl group such as a cyclopropyl group, a cyclopentyl group, a cyclohexyl group, a cyclopropylmethyl group, a 4-methylcyclohexyl group, a cyclohexylmethyl group, a norbornyl group, or an adamantyl group; a C₂-C₂₀ alkenyl group such as a vinyl group, a 1-propenyl group, a 2-propenyl group, a butenyl group, or a hexenyl group; a C₃-C₂₀ cyclic unsaturated hydrocarbyl group such as a cyclohexenyl group; a C₆-C₂₀ aryl group such as a phenyl group or a naphthyl group; a C₇-C₂₀ aralkyl group such as a benzyl group, a 1-phenylethyl group, or a 2-phenylethyl group; and a group obtained by combining these groups. Some or all of hydrogen atoms of the hydrocarbyl group may be replaced with a group containing a heteroatom such as an oxygen atom, a sulfur atom, a nitrogen atom, or a halogen atom, some of (—CH₂—)s in the hydrocarbyl group may be replaced with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like. When d13 is 2, R³³s may be the same as or different from each other.

In addition, when d13 is 2, two R³³s may be bonded to each other to form a ring together with aromatic ring carbon atoms to which they are bonded. Specific examples of the ring formed at this time include a cyclopropane ring, a cyclobutane ring, a cyclopentane ring, a cyclohexane ring, a norbornane ring, and an adamantane ring. Some or all of hydrogen atoms of the ring may be replaced with a group containing a heteroatom such as an oxygen atom, a sulfur atom, a nitrogen atom, or a halogen atom, some of (—CH₂—)s in the ring may be replaced with a group containing a heteroatom such as an oxygen atom, a sulfur atom, or a nitrogen atom, and as a result, the ring may contain a hydroxy group, a fluorine atom, a chlorine atom, a bromine atom, an iodine atom, a cyano group, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like.

In the formula (d2), R³⁴ is a halogen atom other than an iodine atom, a nitro group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. Specific examples of the halogen atom other than an iodine atom include a fluorine atom, a chlorine atom, a bromine atom. The hydrocarbyl group and hydrocarbyl moieties of the hydrocarbyloxy group and the hydrocarbylthio group may be saturated or unsaturated, and may be linear, branched, or cyclic. Specific examples thereof include, but are not limited to, those similar to those exemplified as the hydrocarbyl group of R³³. When d16 is 2, R³⁴s may be the same as or different from each other.

When d16 is 2, two R³⁴s may be bonded to each other to form a ring together with carbon atoms to which they are bonded. The ring is preferably a 5- to 8-membered ring.

In the formula (d2), L^{F1}, L^{F2}, L^{G1}, and L^{G2} are each independently a single bond, an ether bond, an ester bond, a sulfonate bond, an amide bond, a sulfonamide bond, a carbonate bond, or a carbamate bond. Among these, L^{F1} is preferably a single bond, an ether bond, an ester bond, or a sulfonate bond, and more preferably an ester bond or a sulfonate bond. L^{F2} is preferably a single bond, an ether bond, an ester bond, or a sulfonate bond, and more preferably an ester bond or a sulfonate bond. L^{G1} is preferably a single bond, an ether bond, an ester bond, or a sulfonate bond, and more preferably a single bond, an ether bond, or an ester bond. L^{G2} is preferably a single bond, an ether bond, an ester bond, or a sulfonate bond, and more preferably a single bond, an ether bond, or an ester bond.

L^{F1} and L^{F2} are preferably bonded to adjacent aromatic ring carbon atoms. At this time, since the substituent including a fluorosulfonic acid anion structure and the substituent including an aromatic ring substituted with an iodine atom are present at spatially closer positions, higher sensitivity is expected.

In the formula (d2), X^{L4} and X^{L5} are each independently a single bond or a C₁-C₄₀ hydrocarbylene group which may contain a heteroatom. The hydrocarbylene group may be linear, branched, or cyclic, and specific examples thereof include an alkanediyl group, a cyclic saturated hydrocarbylene group, and an arylene group. Specific examples of the heteroatom include an oxygen atom, a nitrogen atom, and a sulfur atom. Specific examples of the C₁-C₄₀ hydrocarbylene group of X^{L4} and X^{L5} which may contain a heteroatom include X^{L}-0 to X^{L}-61 exemplified as specific examples of the C₁-C₄₀ hydrocarbylene group of X^{L1} and X^{L2} which may contain a heteroatom in the description of the formula (Z). Among these, X^{L}-0 to X^{L}-22, X^{L}-29 to X^{L}-34, and X^{L}-47 to X^{L}-61 are preferable.

In the formula (d2), Q¹¹ and Q¹² are each independently a hydrogen atom, a fluorine atom, or a C₁-C₆ fluorinated saturated hydrocarbyl group. The C₁-C₆ fluorinated saturated hydrocarbyl group is preferably a trifluoromethyl group.

In the formula (d2), Q¹³ and Q¹⁴ are each independently a fluorine atom or a C₁-C₆ fluorinated saturated hydrocarbyl group. The C₁-C₆ fluorinated saturated hydrocarbyl group is preferably a trifluoromethyl group. Q¹³ and Q¹⁴ are each more preferably a fluorine atom. Specific preferable examples of a partial structure of—[C(Q¹¹) (Q¹²)]_{d17}—C(Q¹³) (Q¹⁴)—SO₃⁻ in the formula (d2) include, but are not limited to, those listed below. Note that, in the following formulas, * represents a bond with L^{G1}.

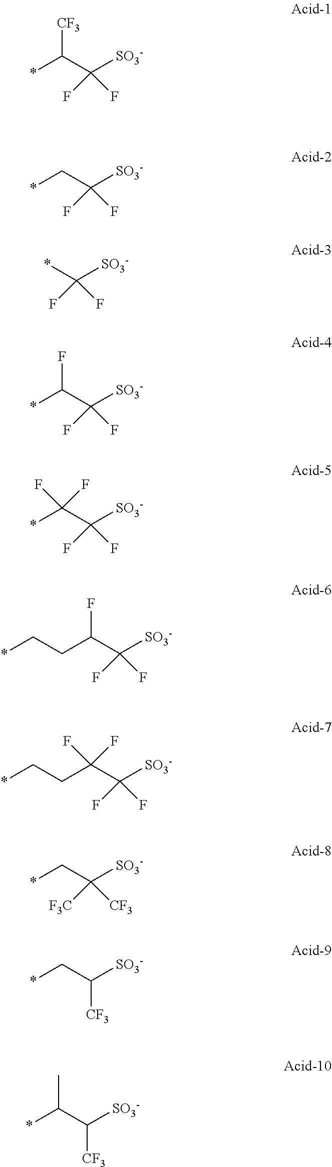

Among these, Acid-1 to Acid-7 are preferable, and Acid-1 to Acid-3, Acid-6, and Acid-7 are more preferable.

The anion having the formula (d2) preferably has the following formula (d2-1):

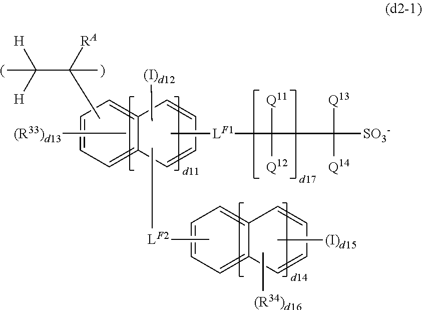

wherein R^{A}, R³³, R³⁴, L^{F1}, L^{F2}, Q¹¹ to Q¹⁴, and d11 to d17 are as defined above.

The anion having the formula (d2-1) preferably has the following formula (d2-2):

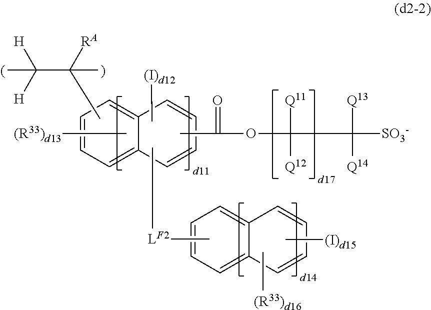

wherein R^{A}, R³³, R³⁴, L^{F2}, Q¹¹, Q¹², and d11 to d17 are as defined above.

Specific examples of the anion having the formula (d2) include, but are not limited to, those listed below. Note that, in the following formulas, R^{A} and Q¹¹ are as defined above, and Me is a methyl group. Bonding positions of various substituents on an aromatic ring may be replaced with each other.

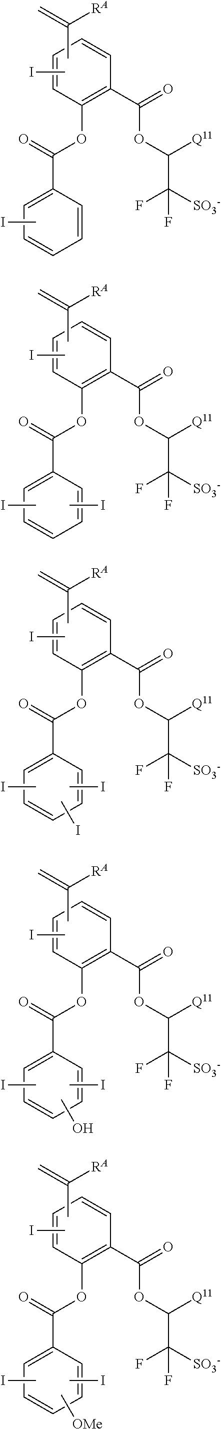

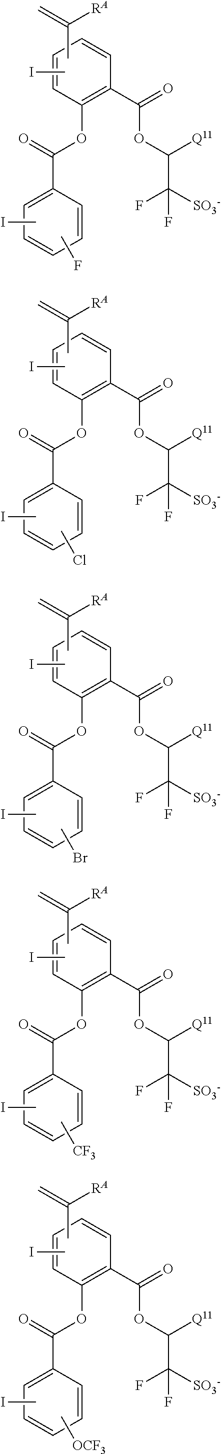

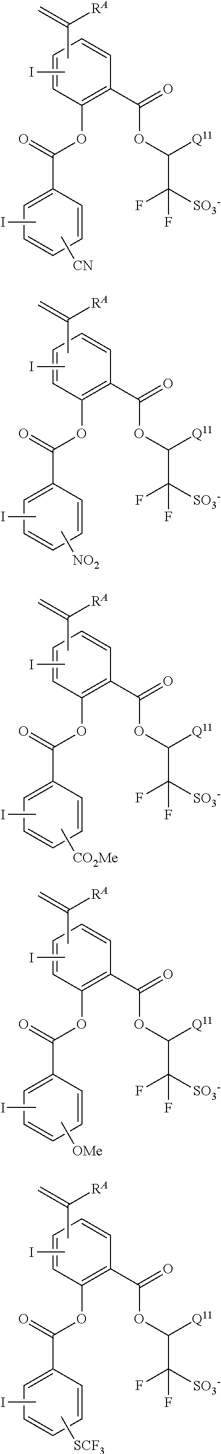

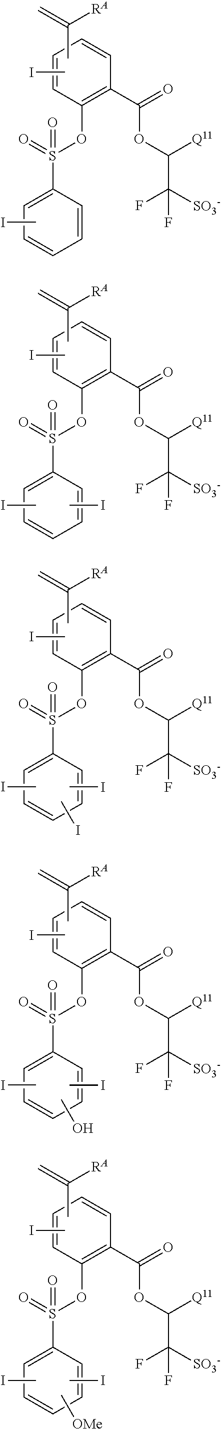

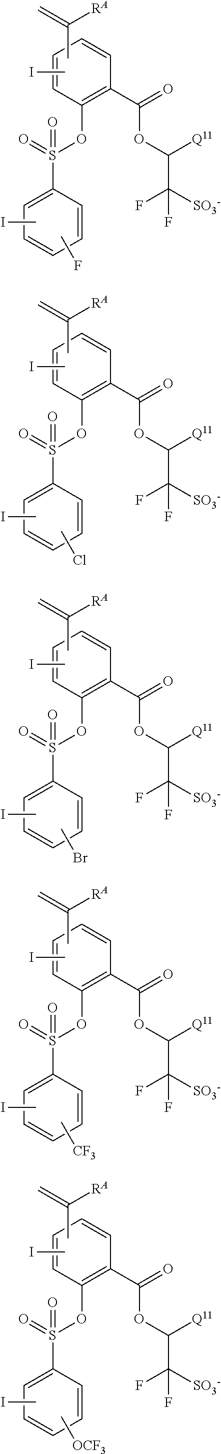

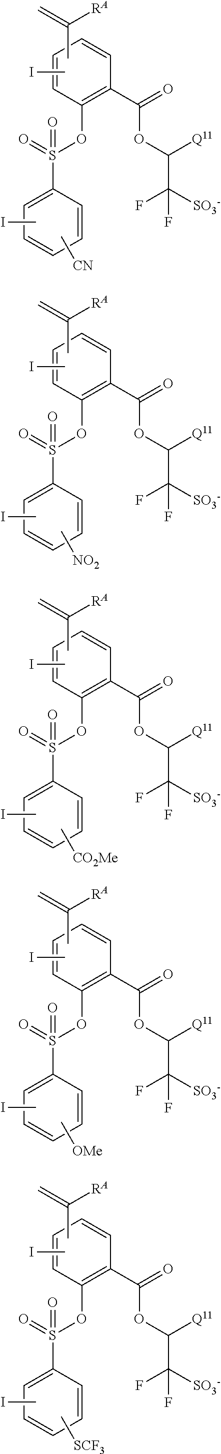

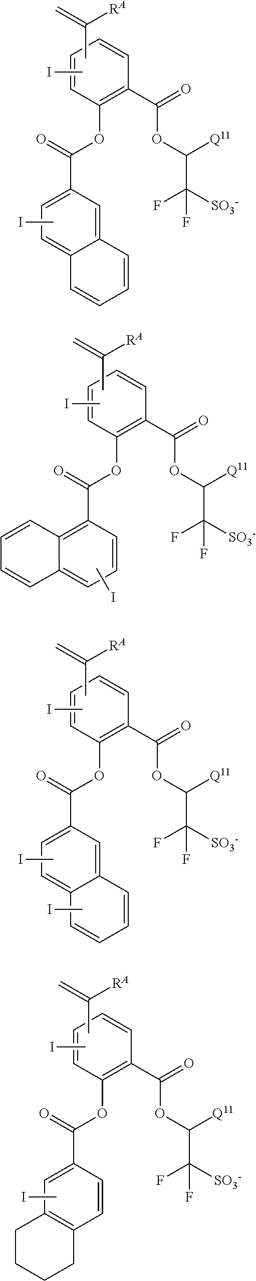

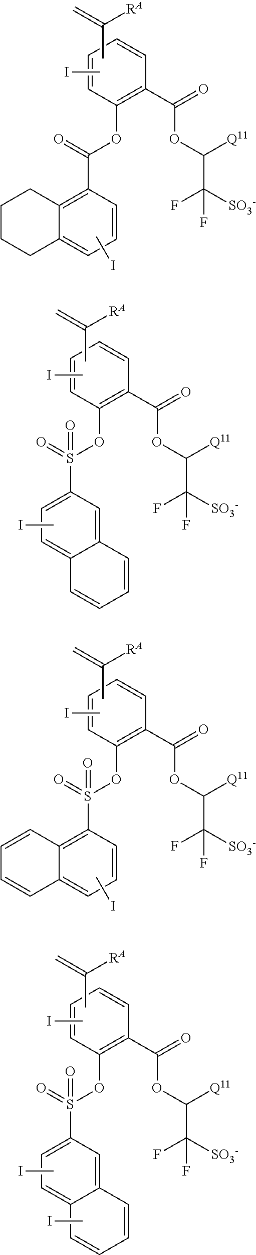

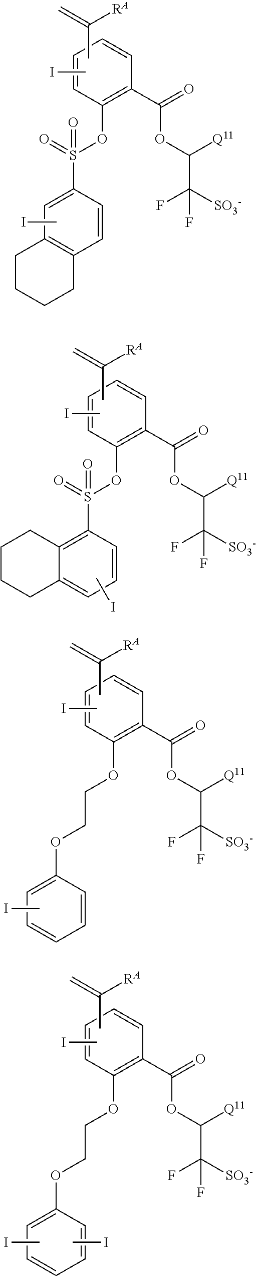

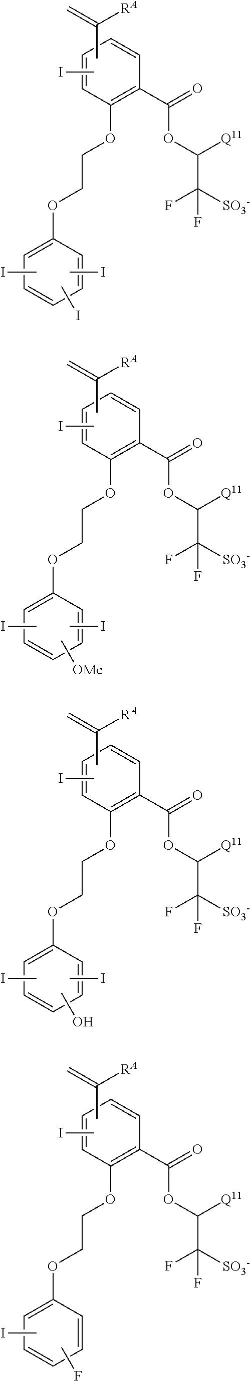

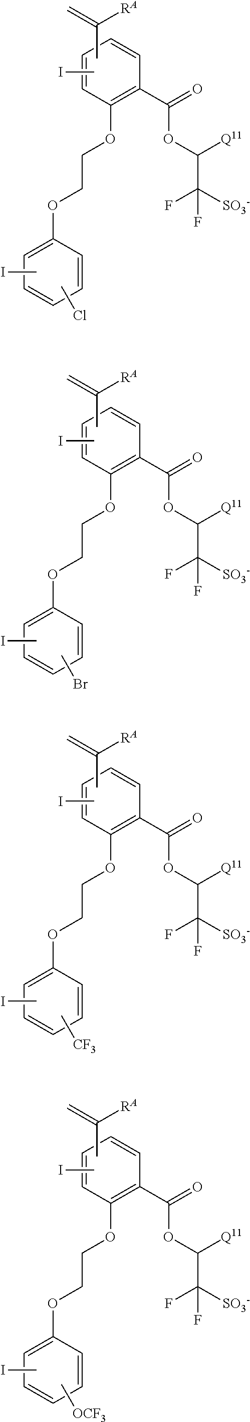

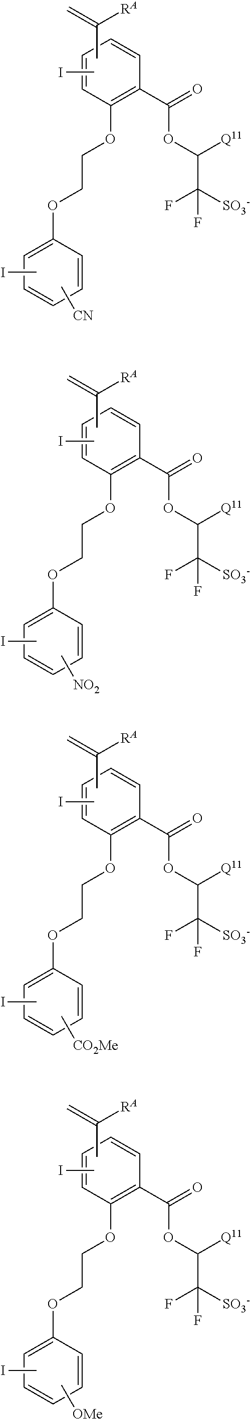

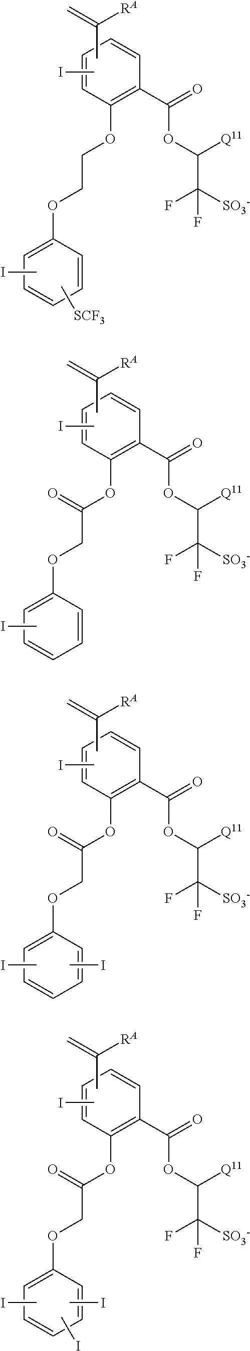

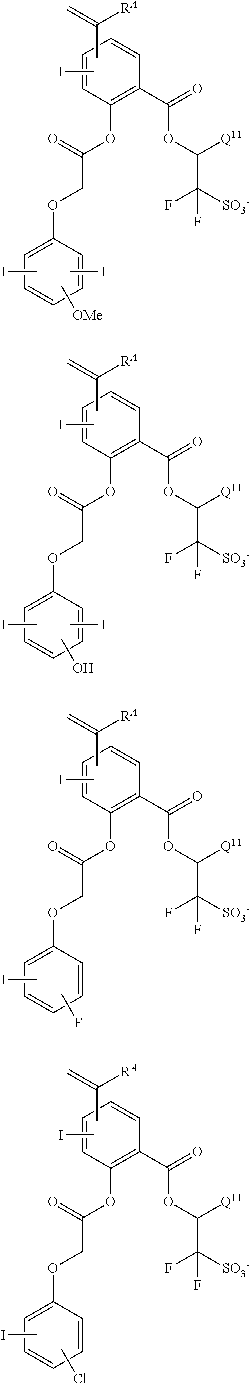

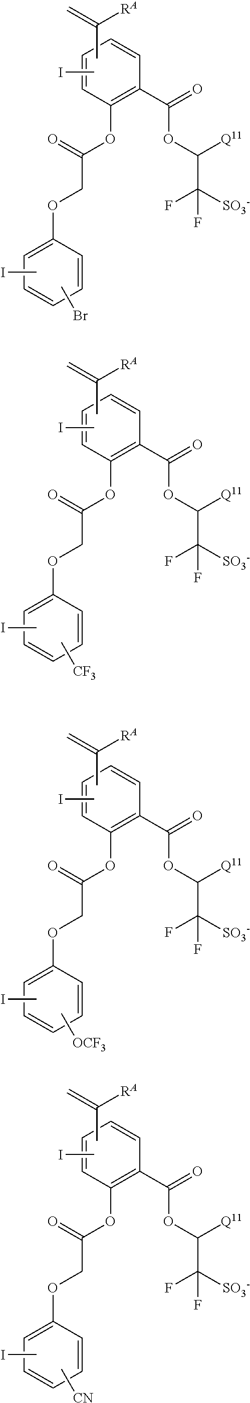

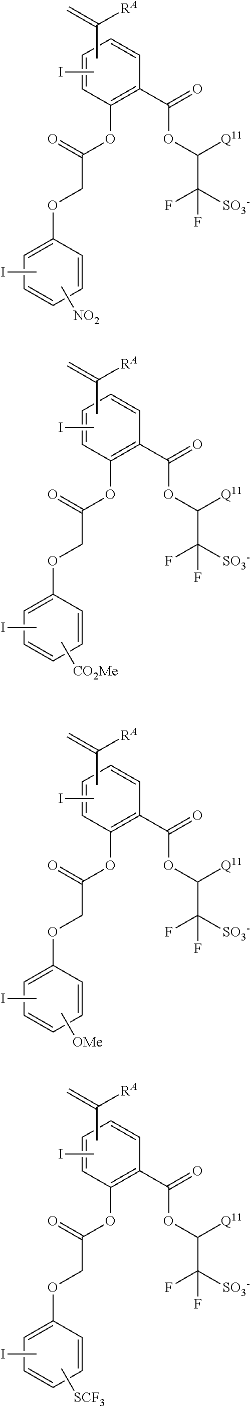

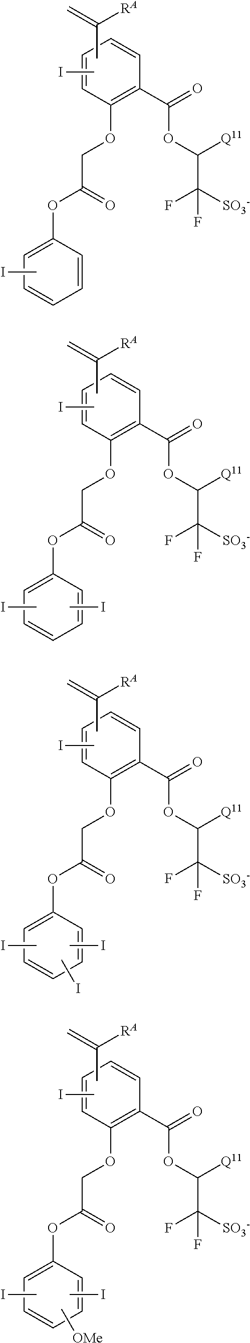

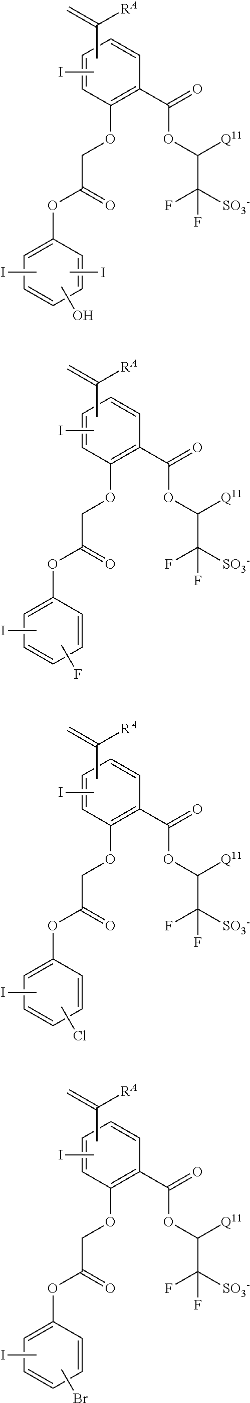

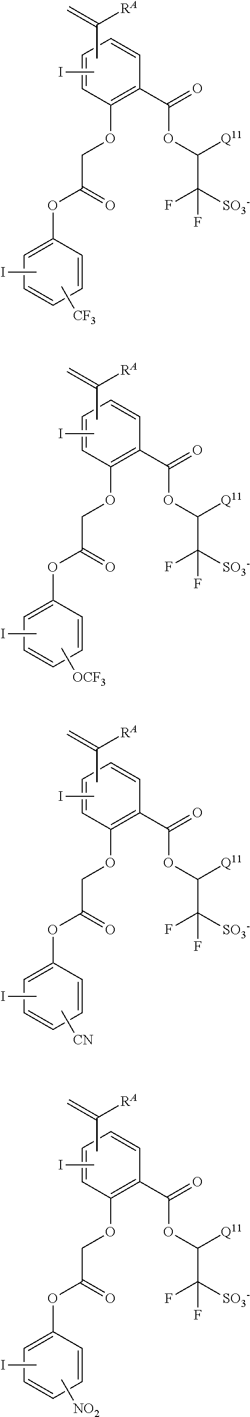

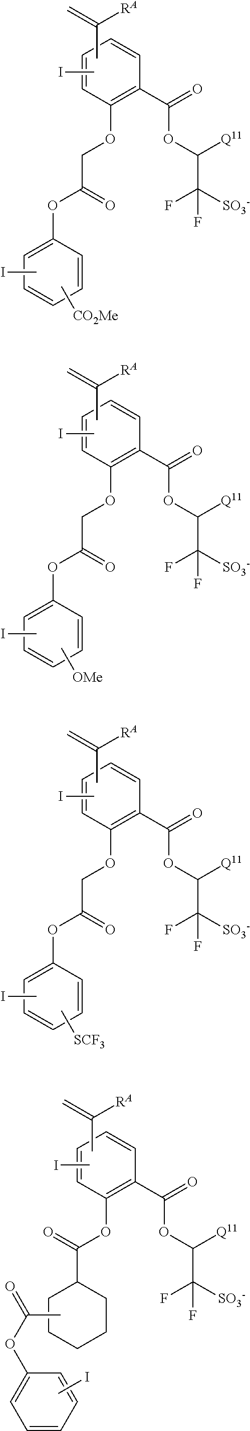

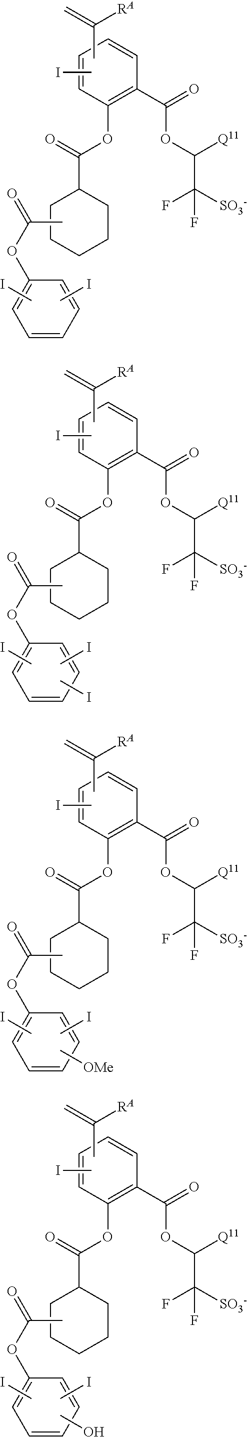

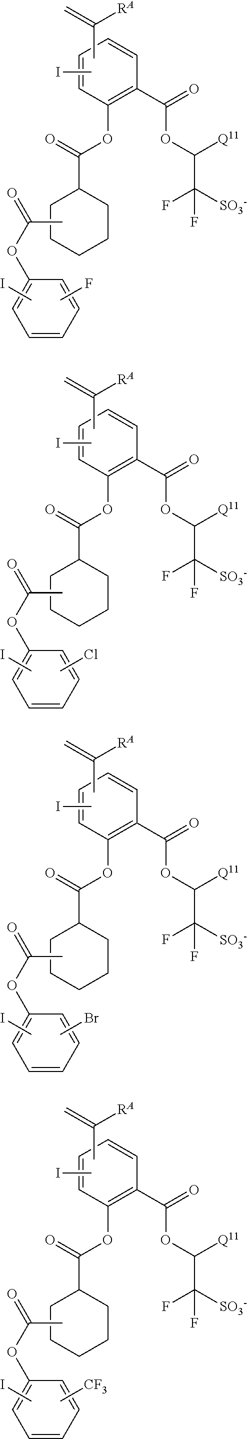

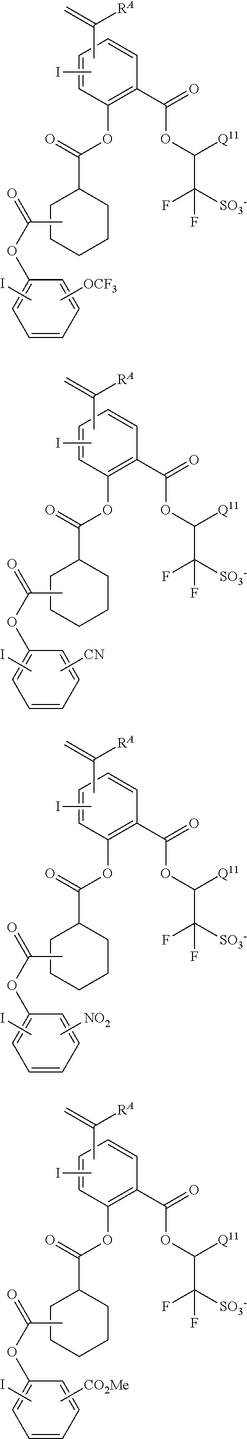

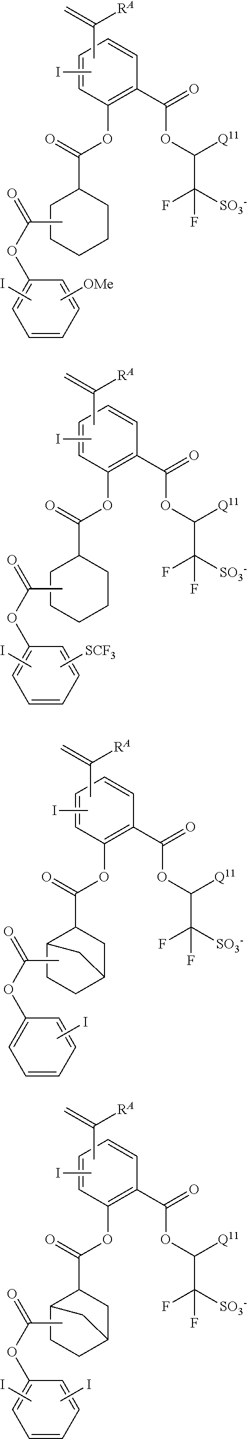

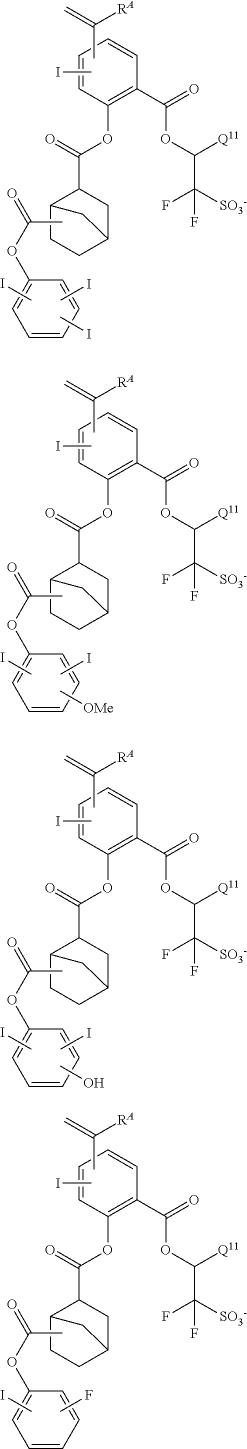

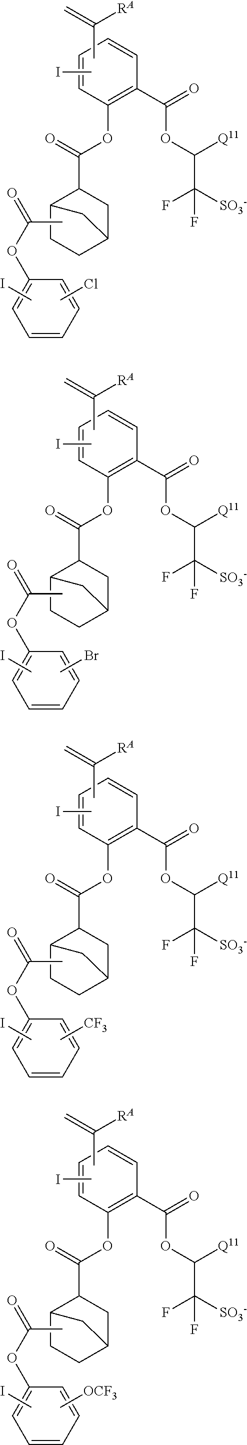

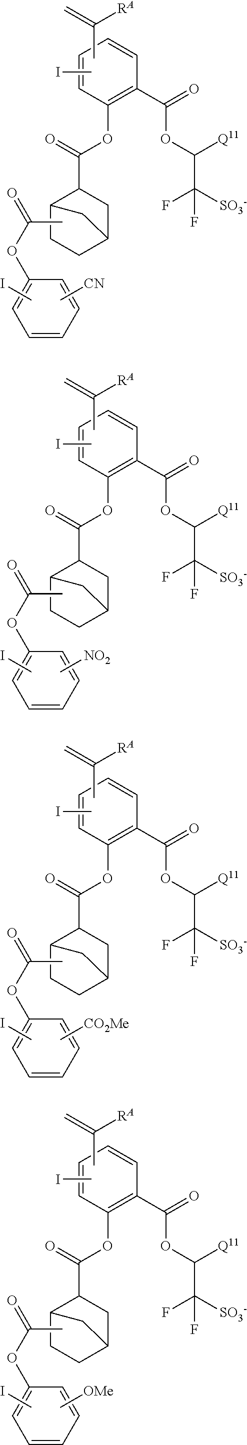

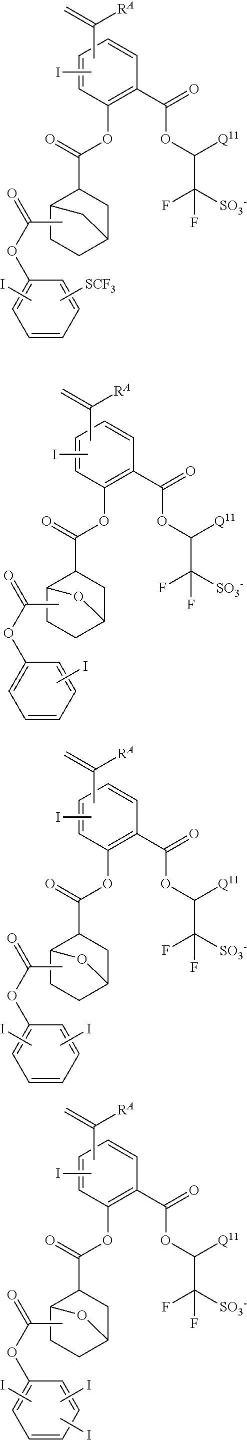

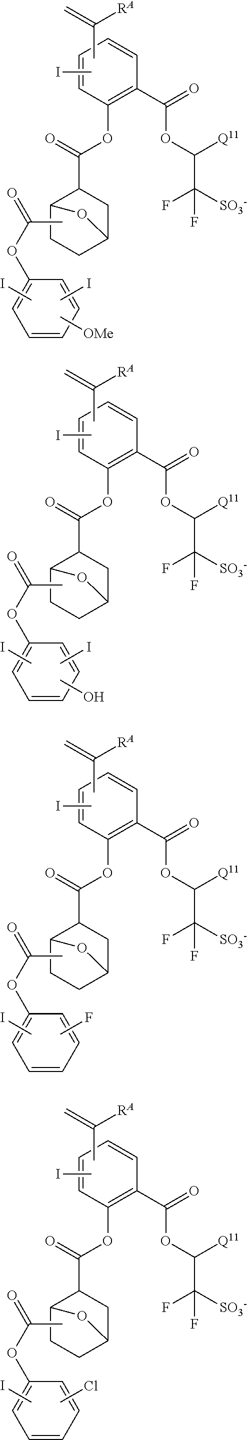

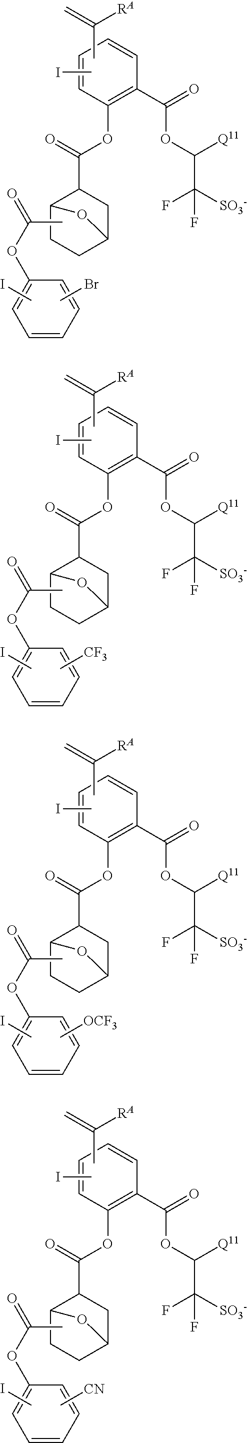

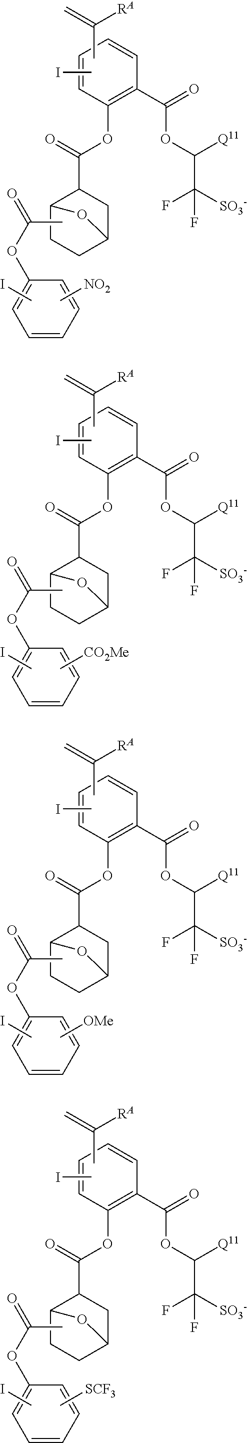

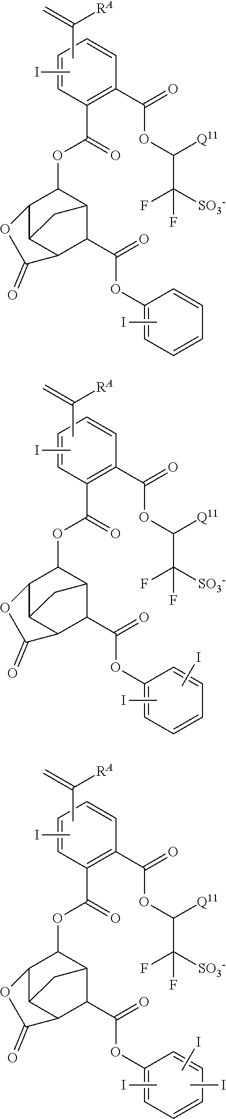

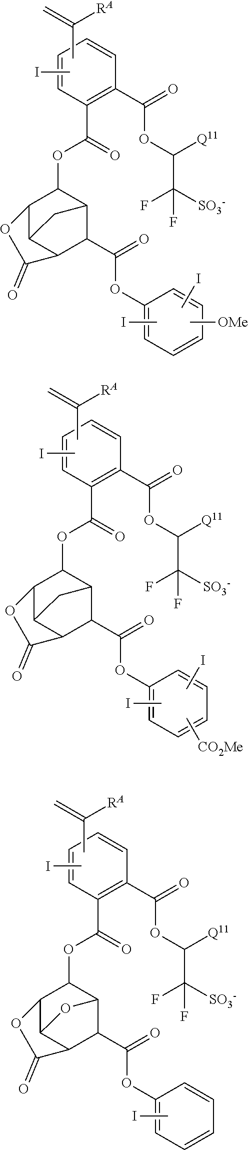

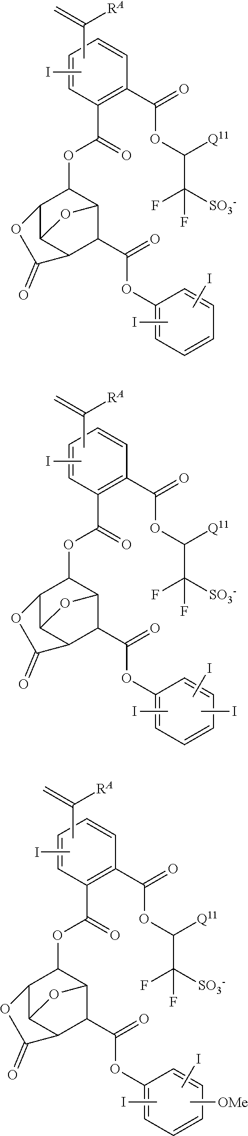

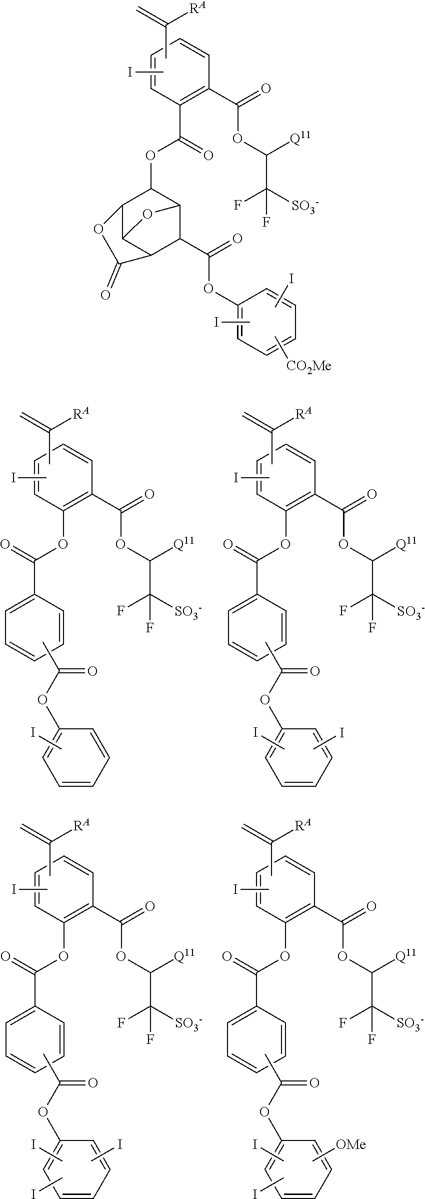

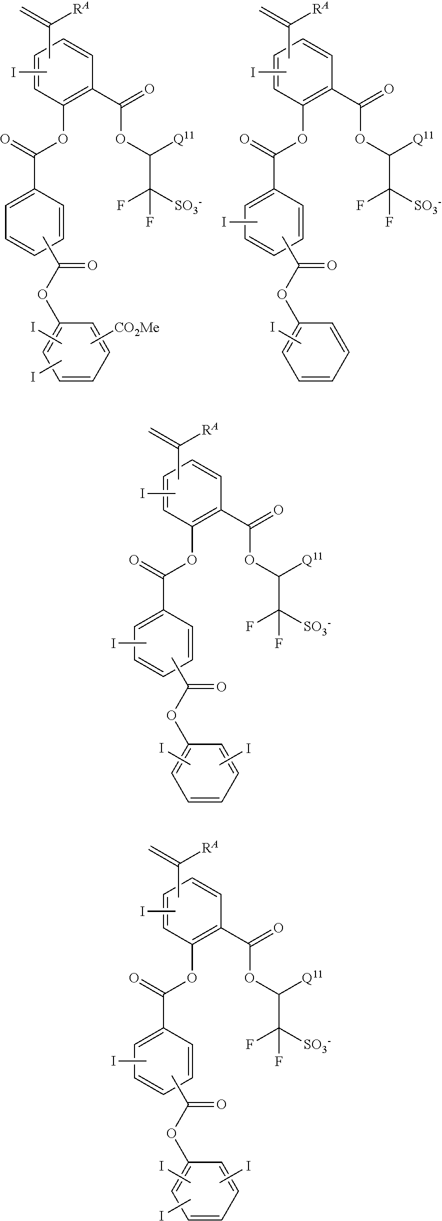

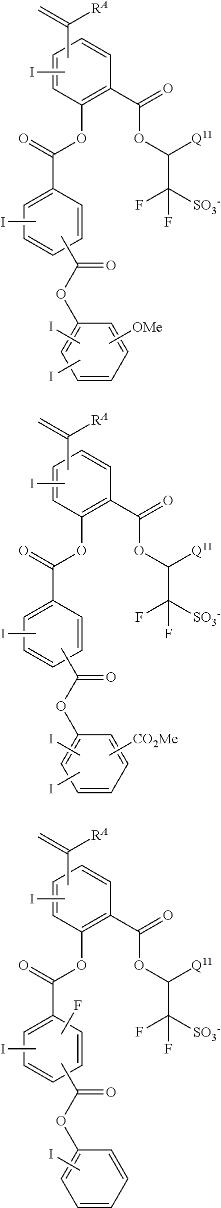

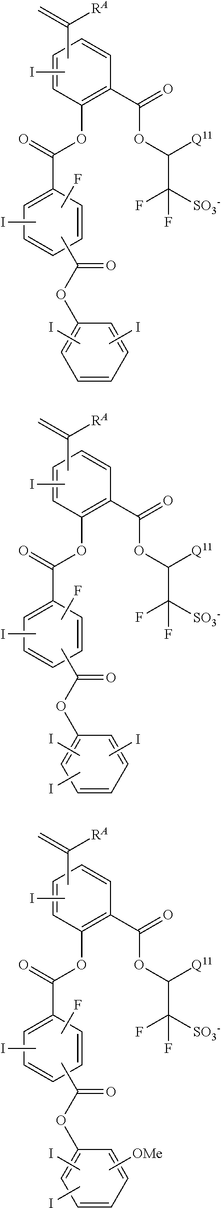

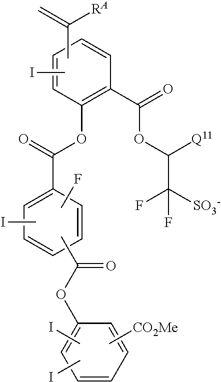

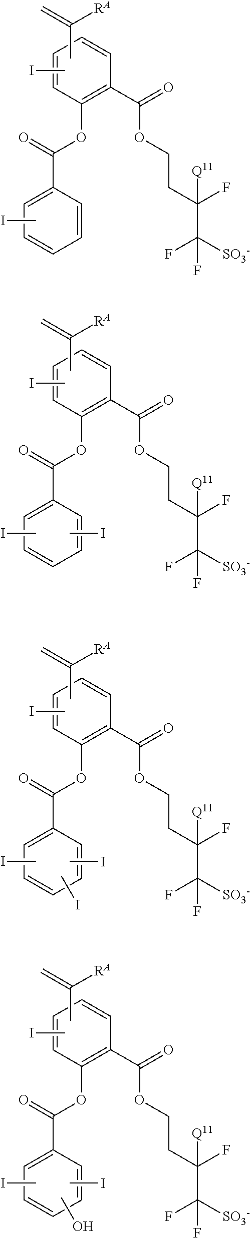

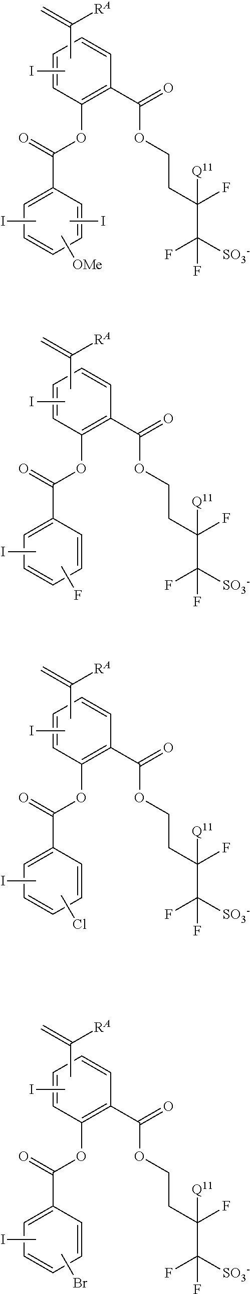

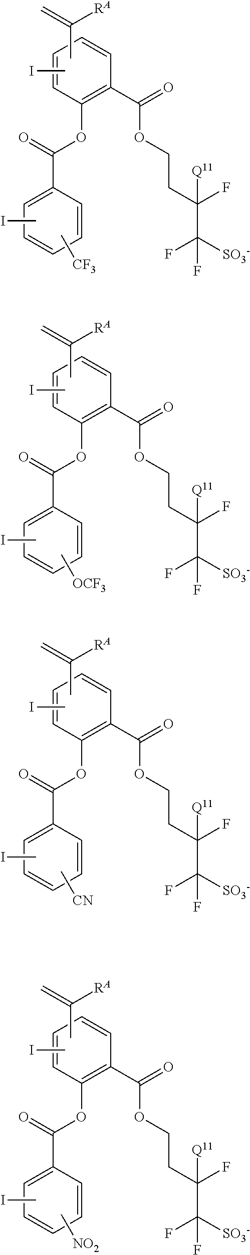

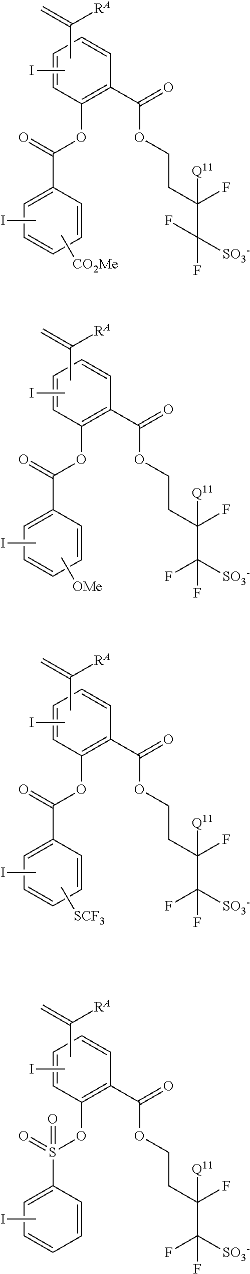

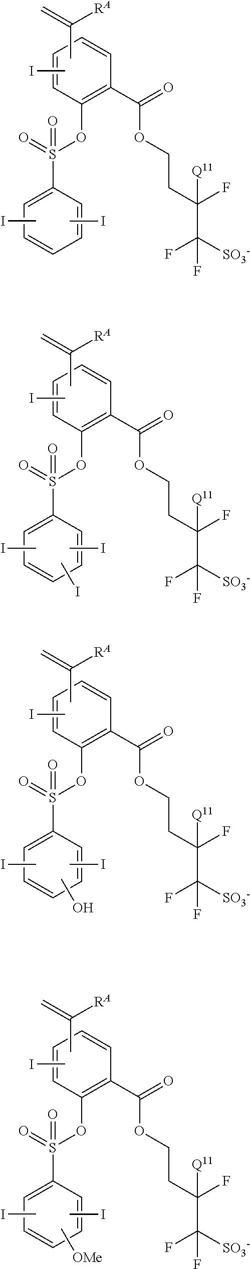

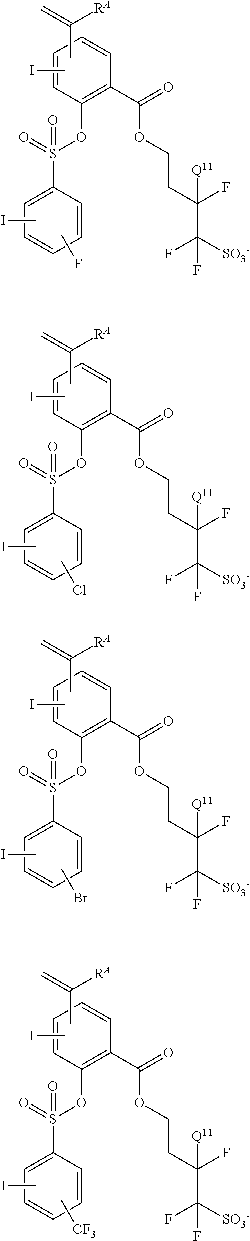

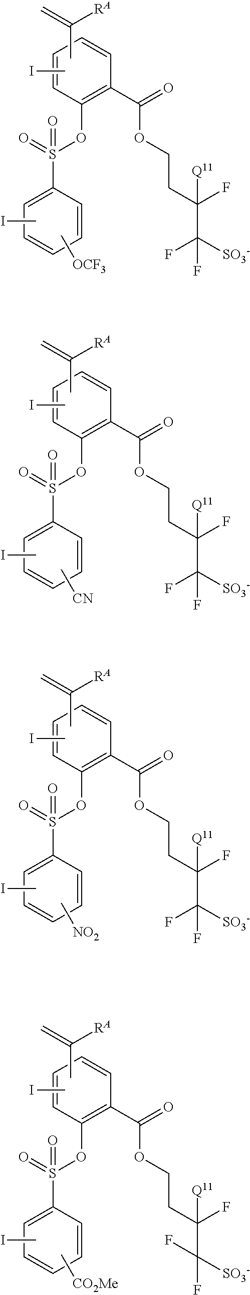

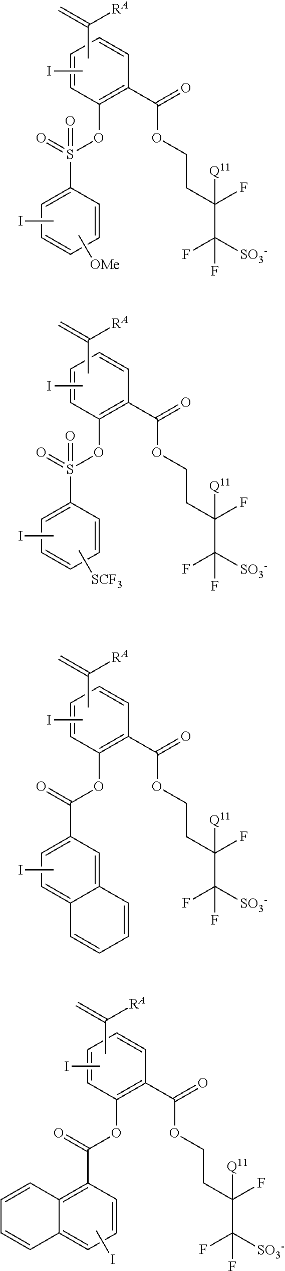

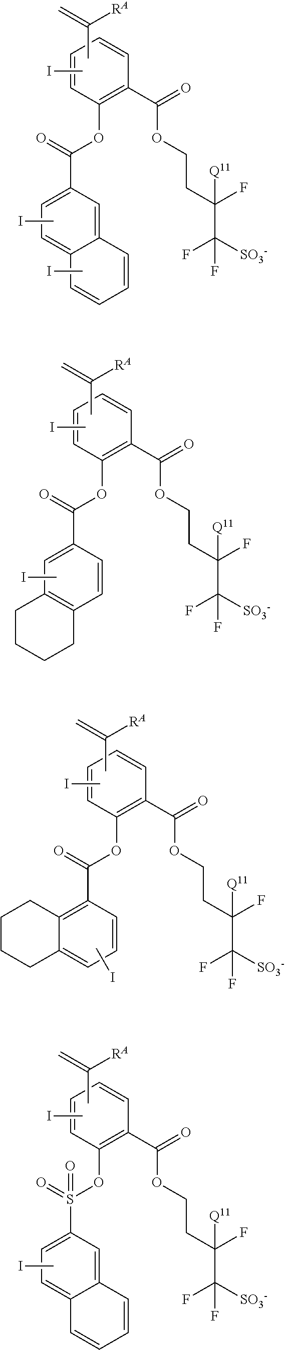

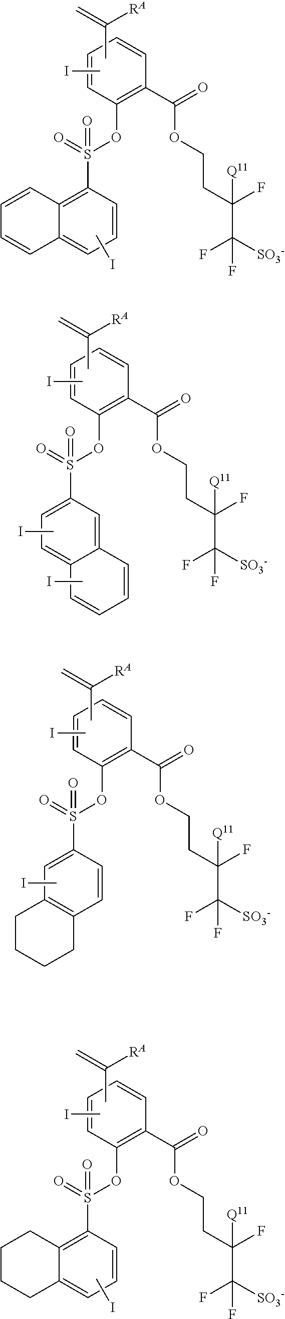

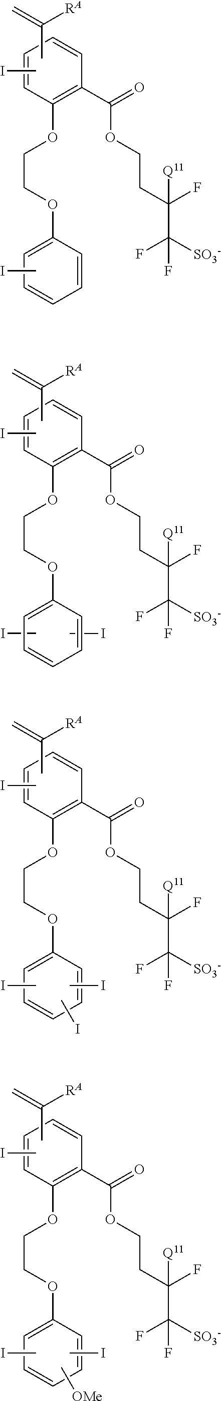

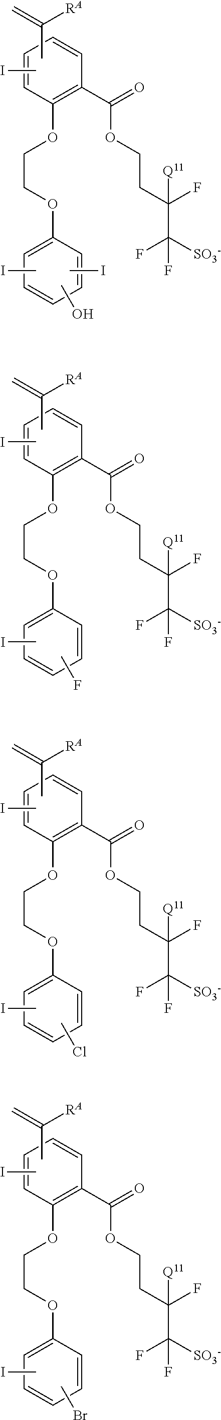

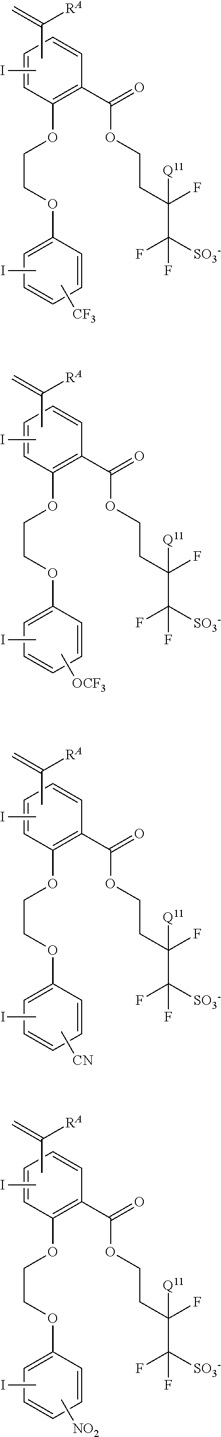

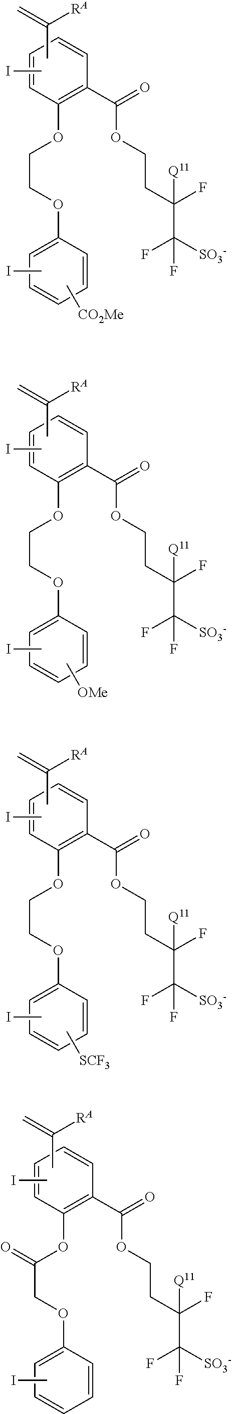

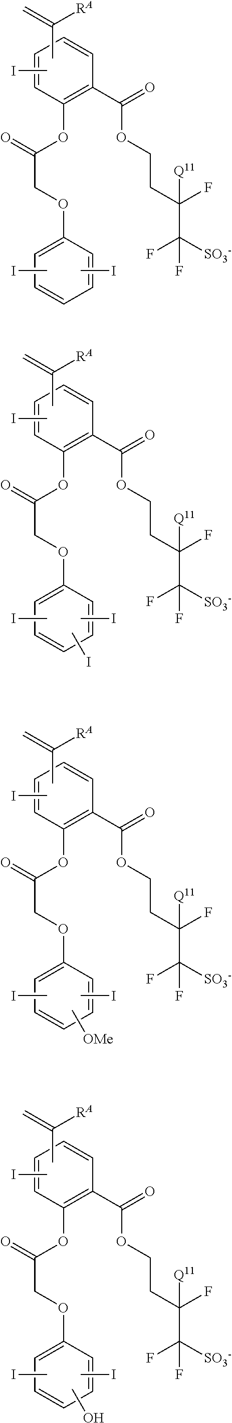

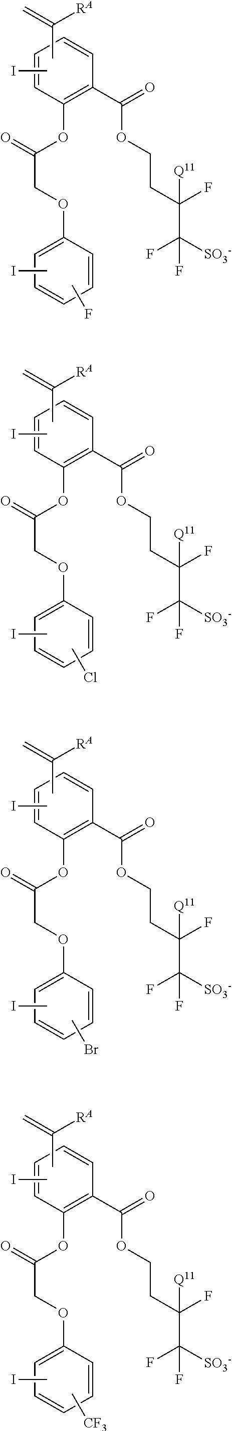

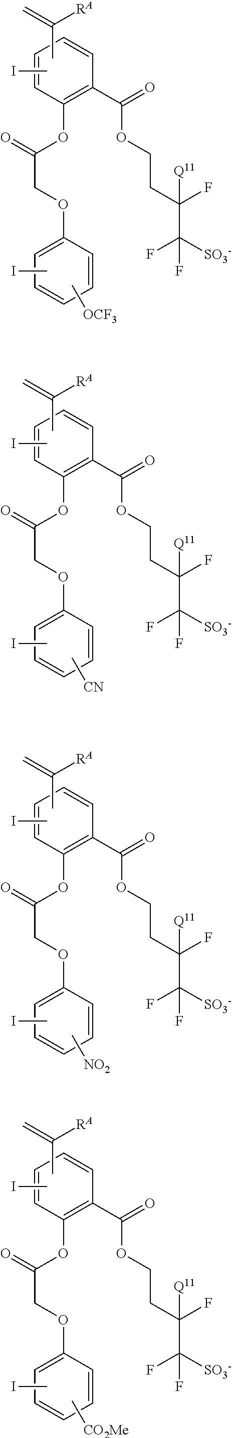

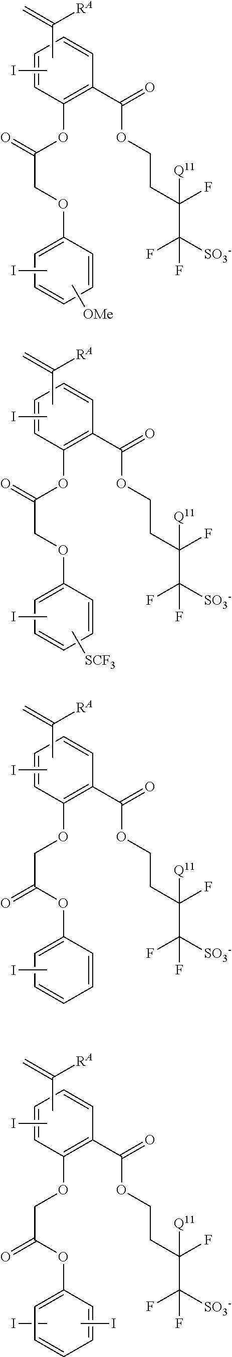

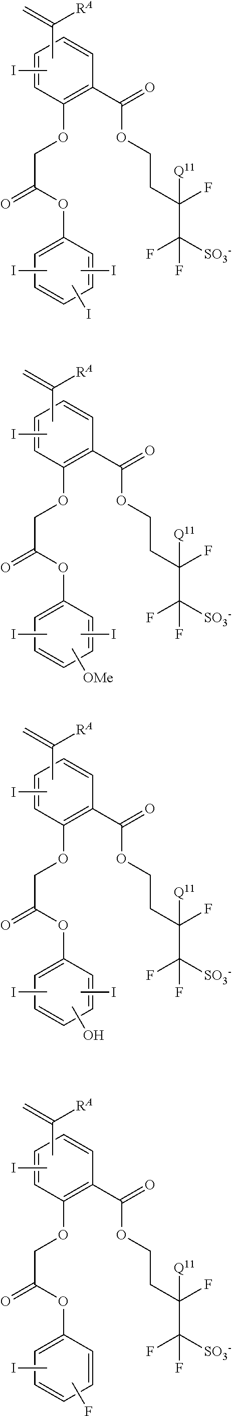

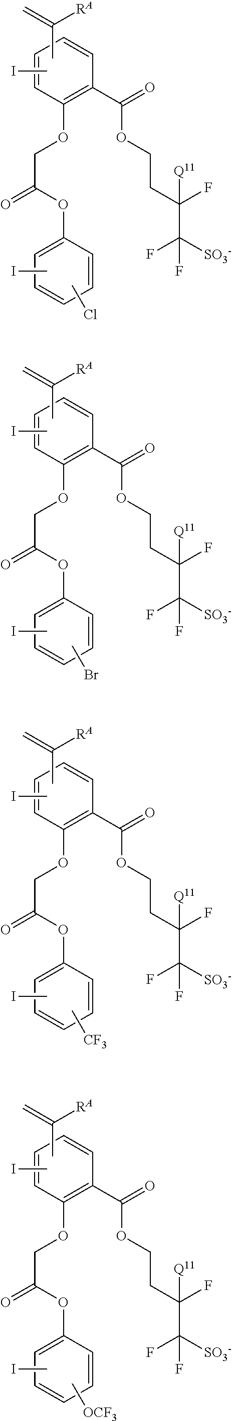

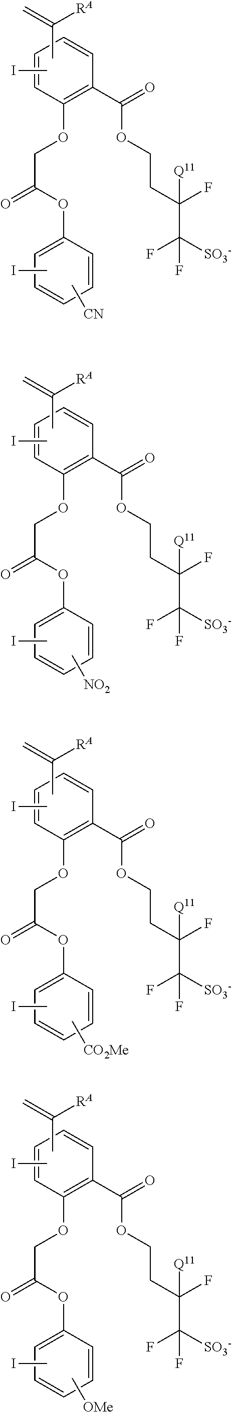

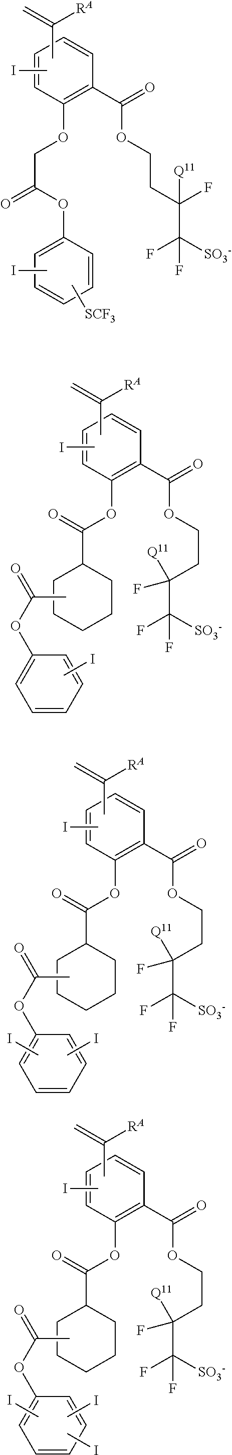

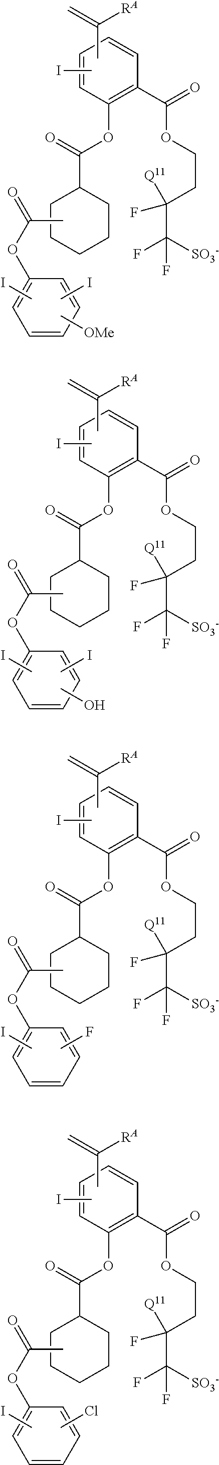

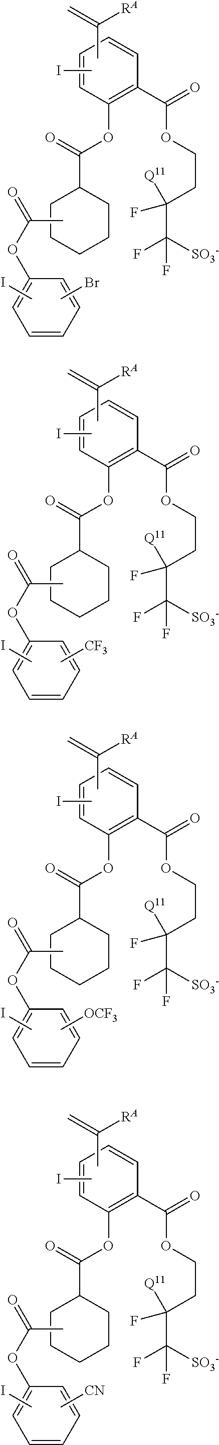

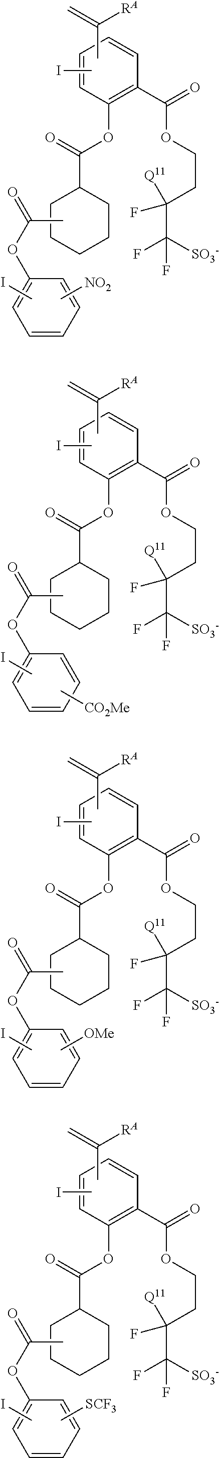

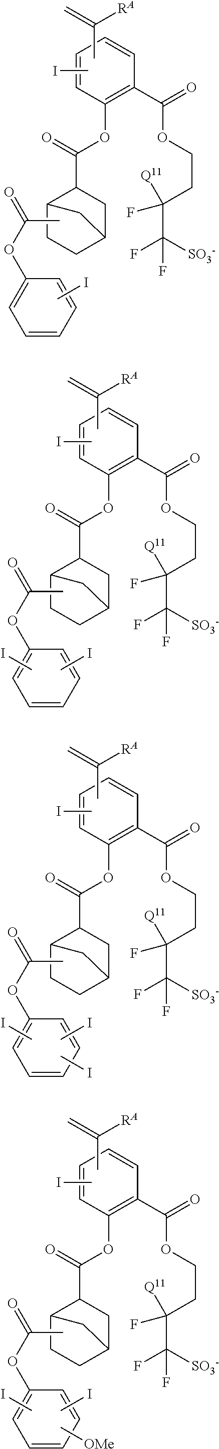

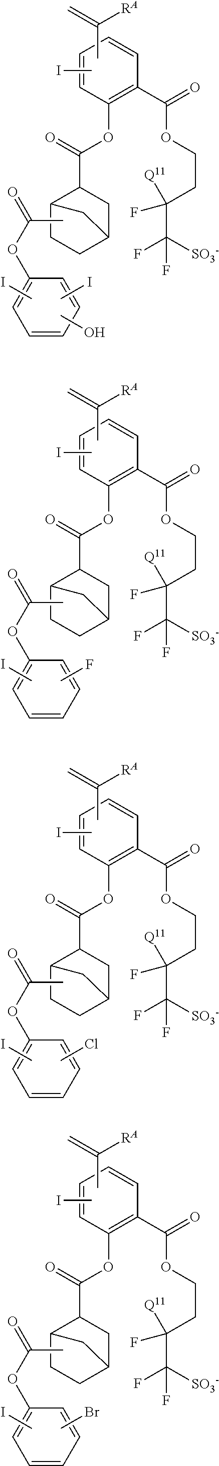

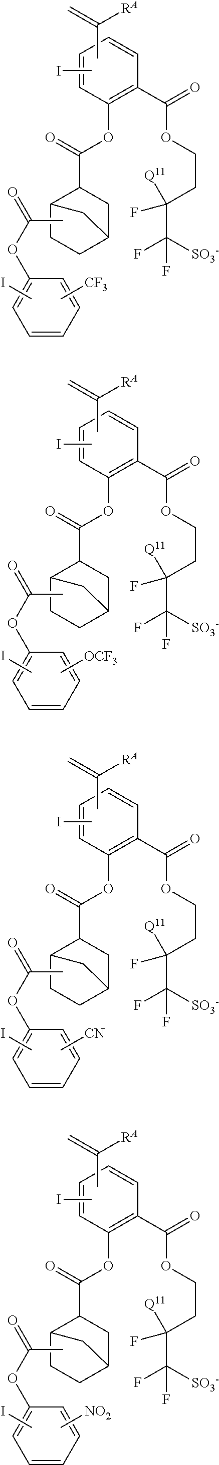

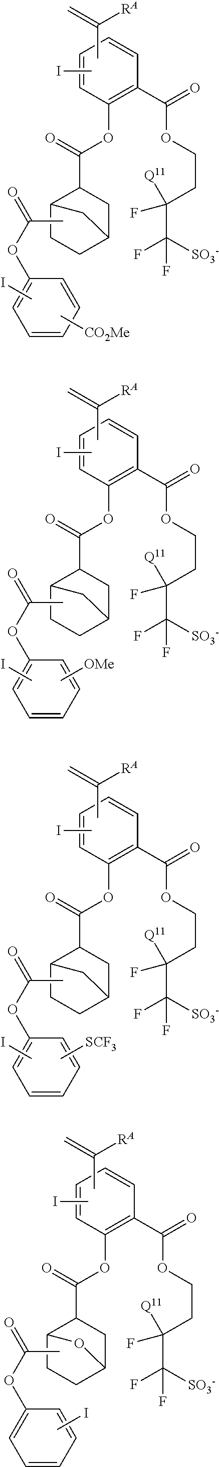

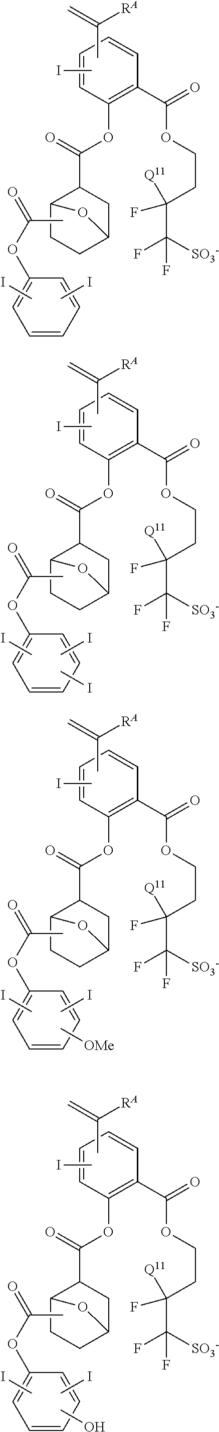

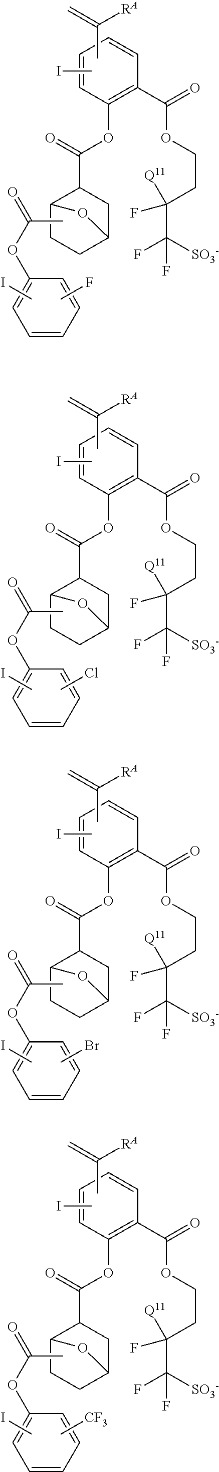

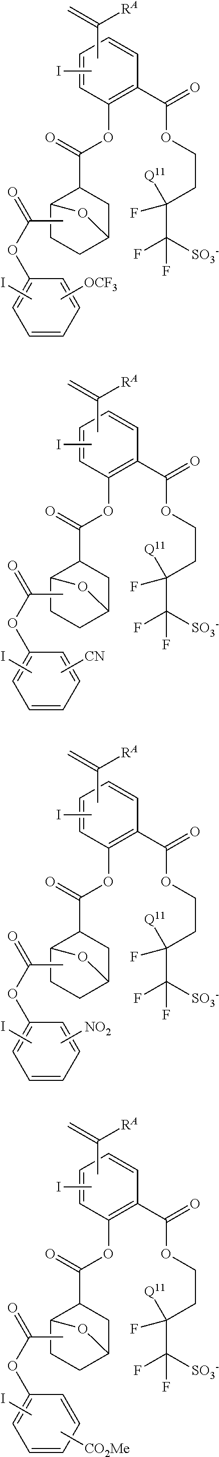

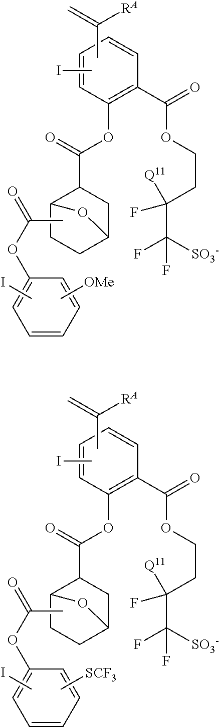

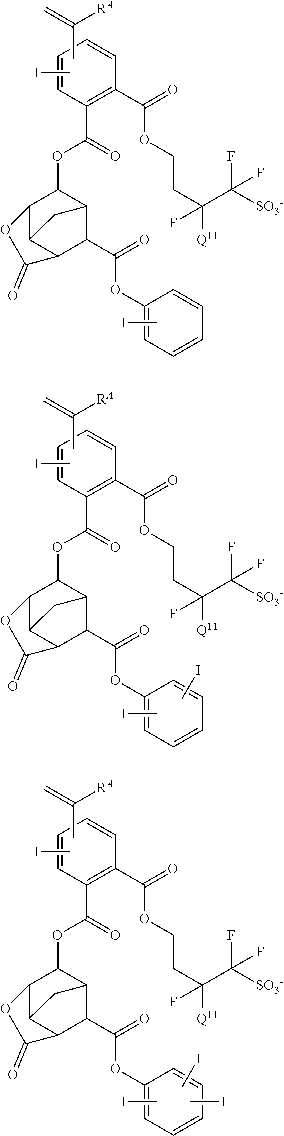

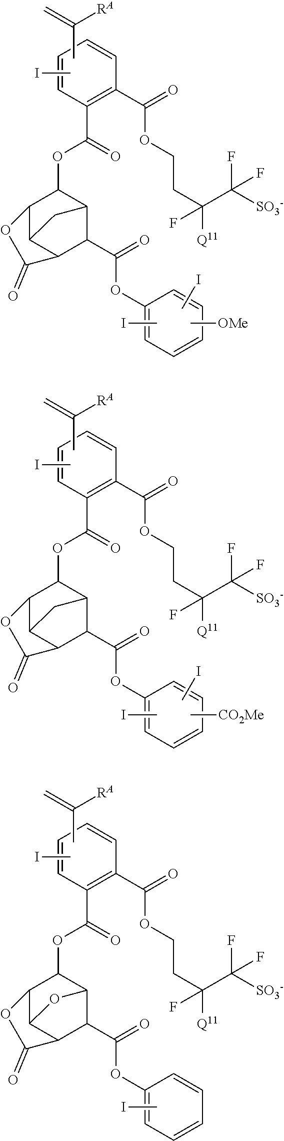

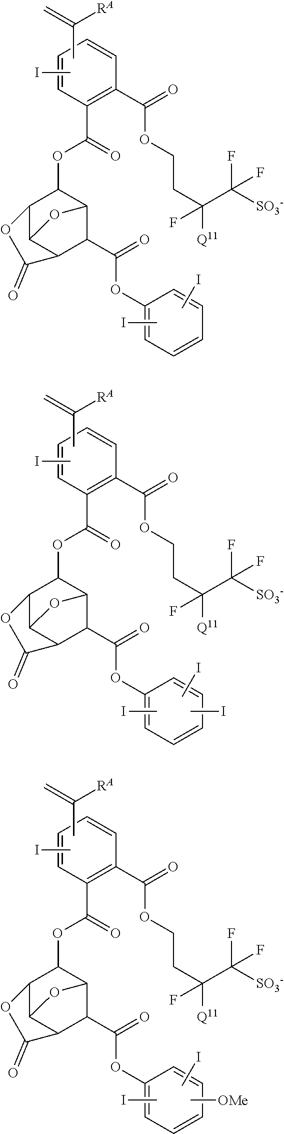

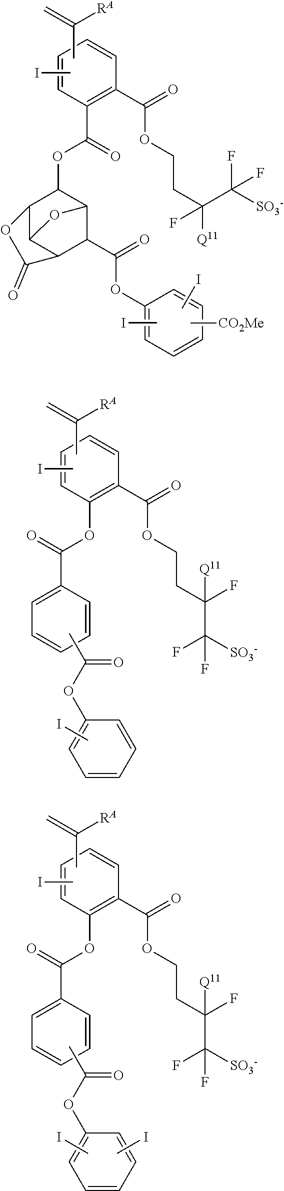

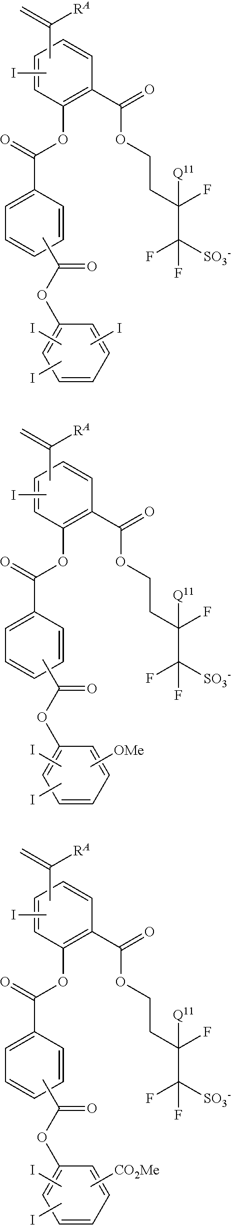

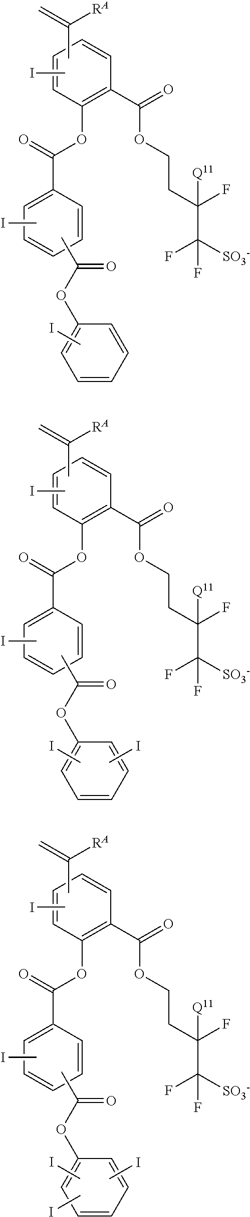

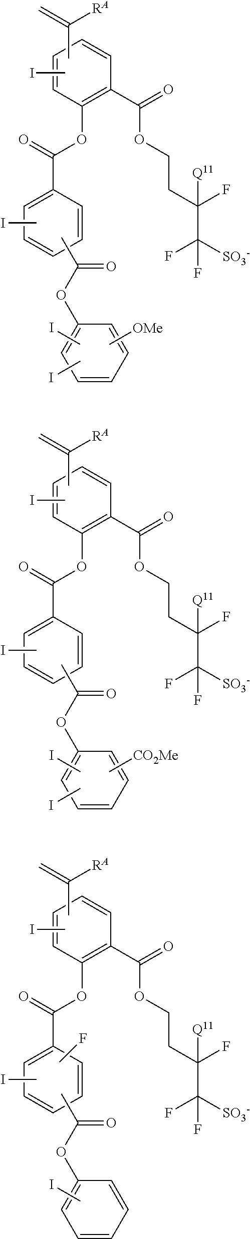

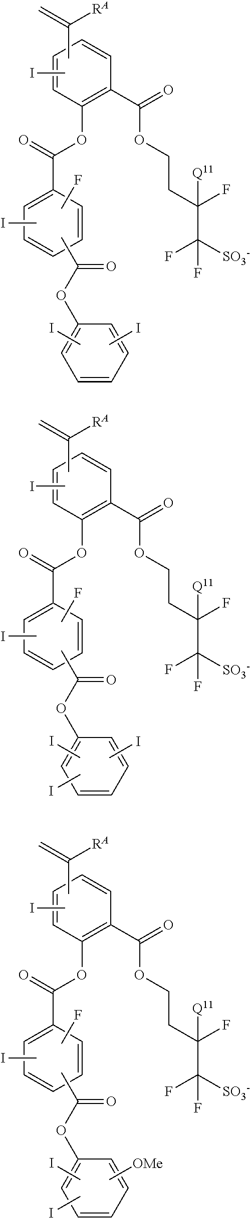

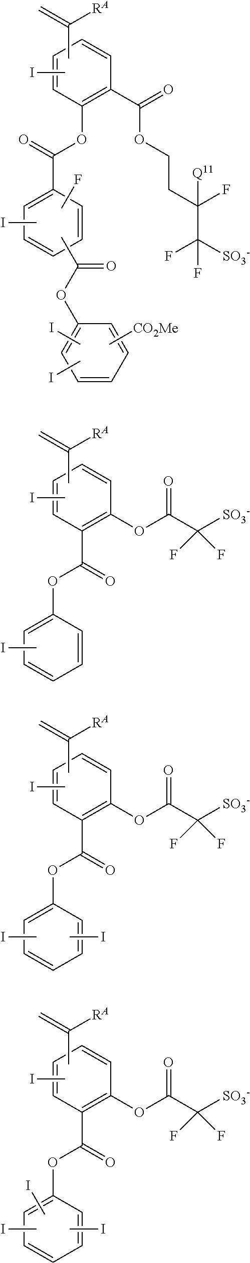

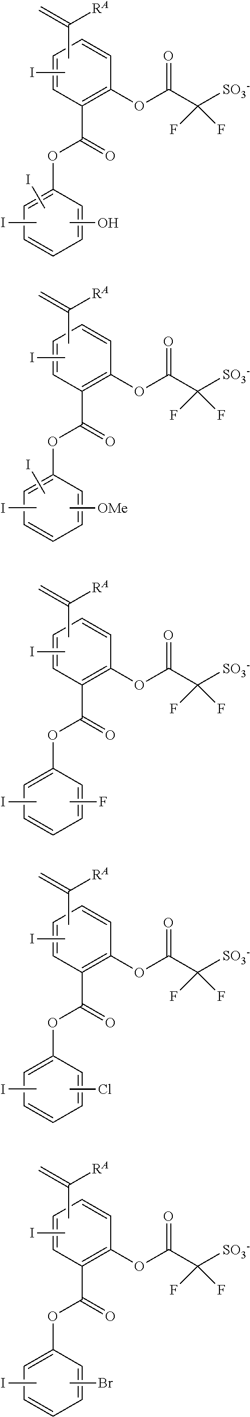

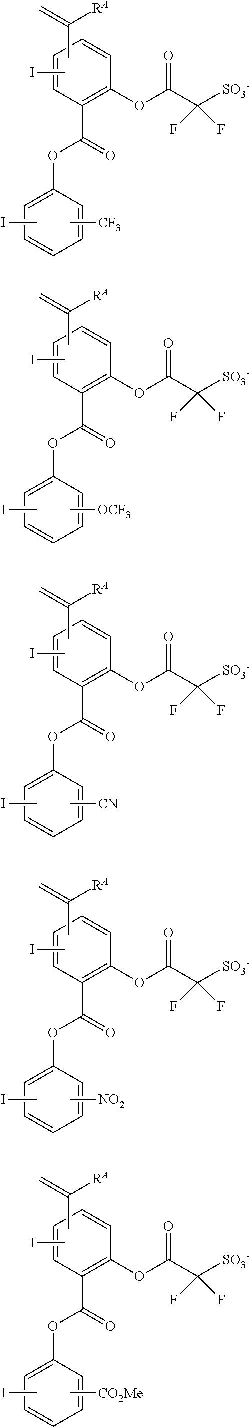

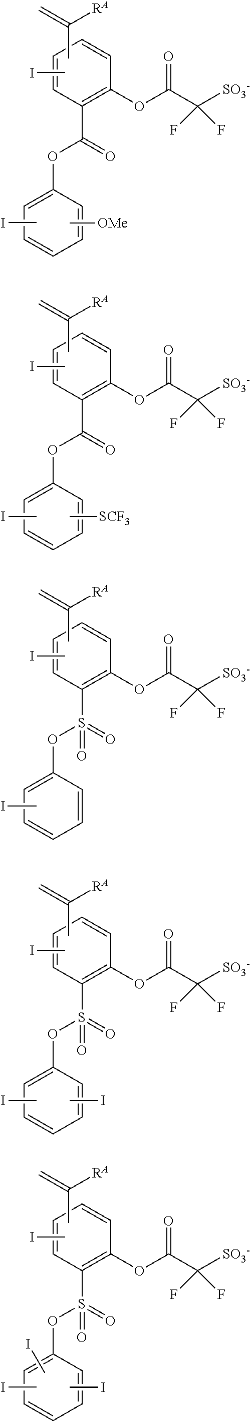

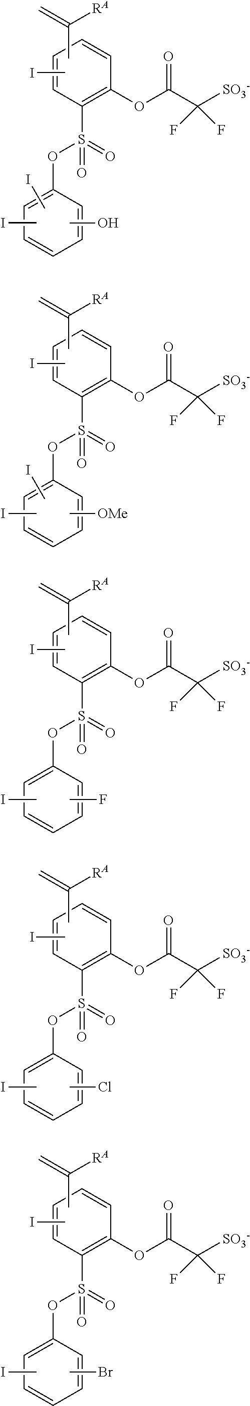

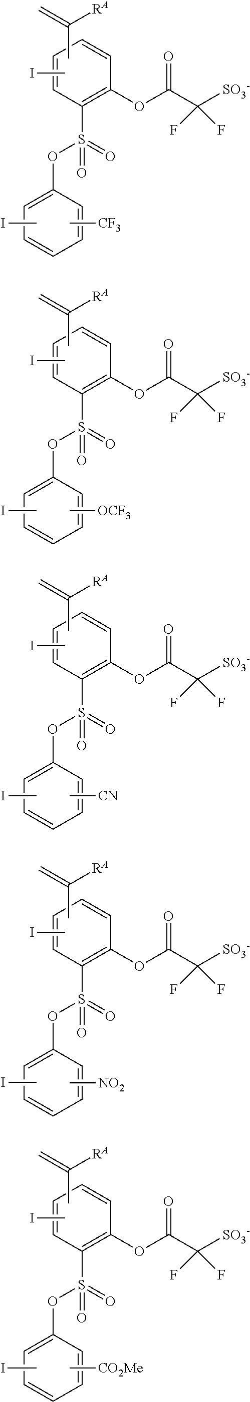

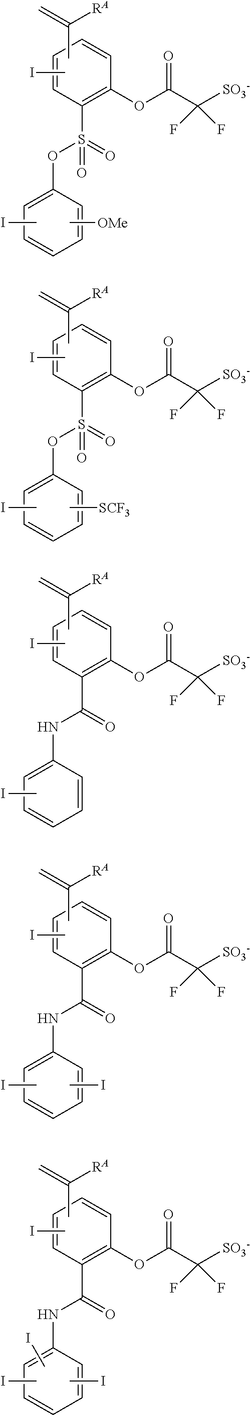

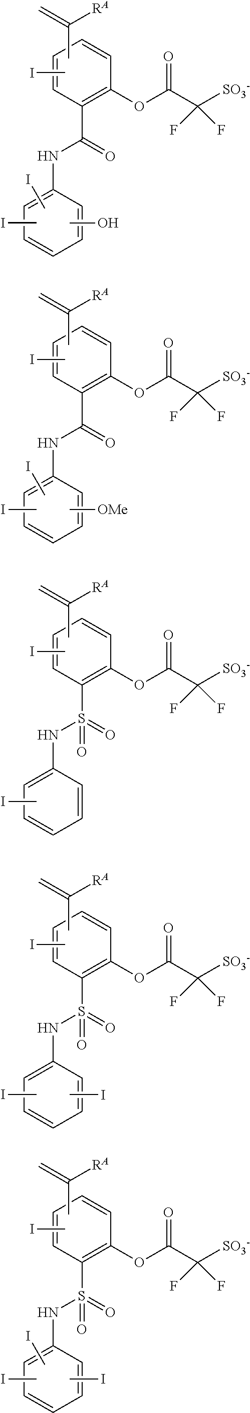

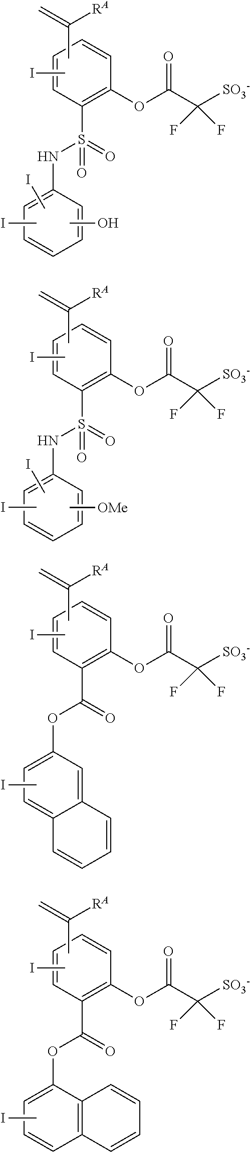

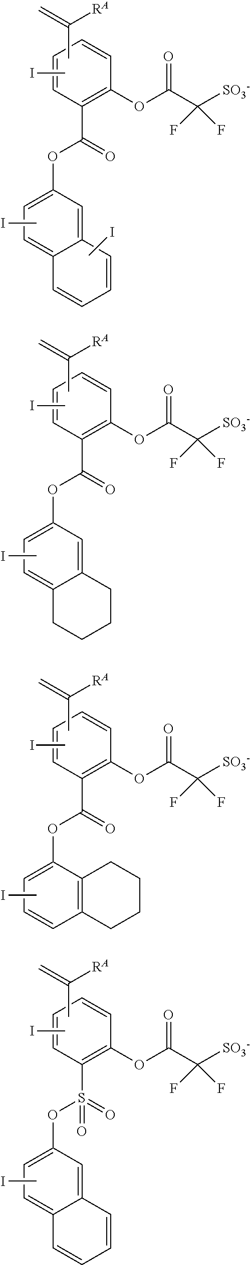

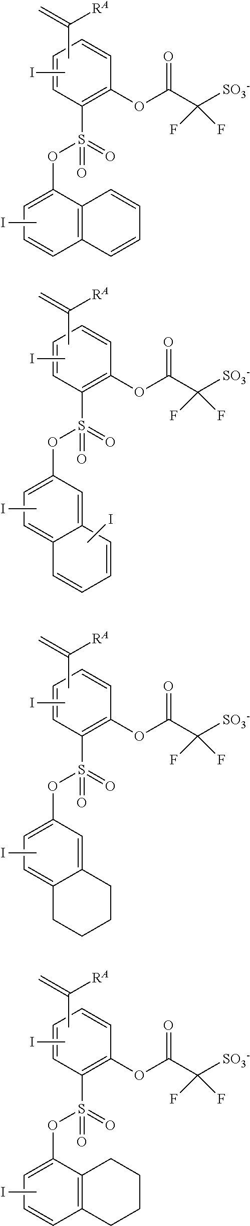

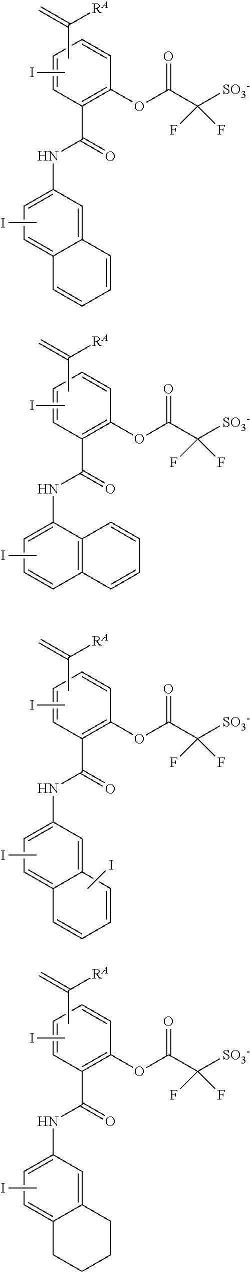

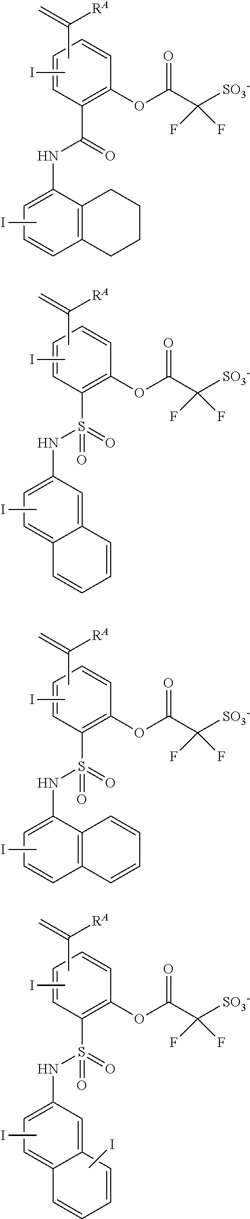

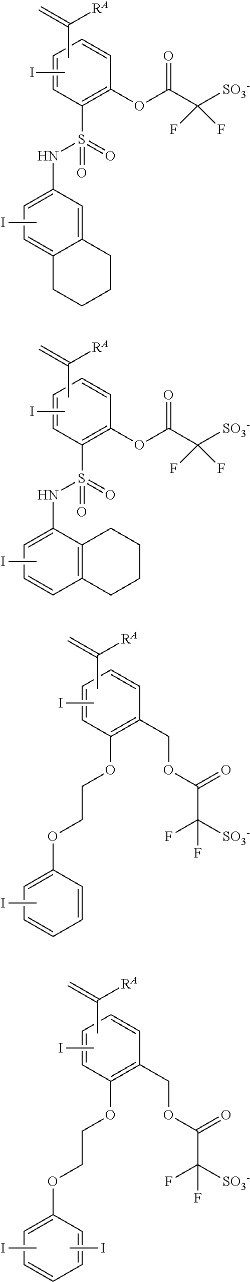

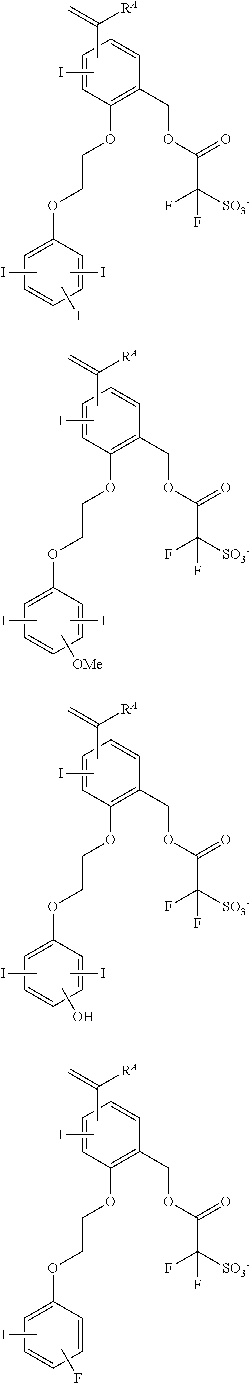

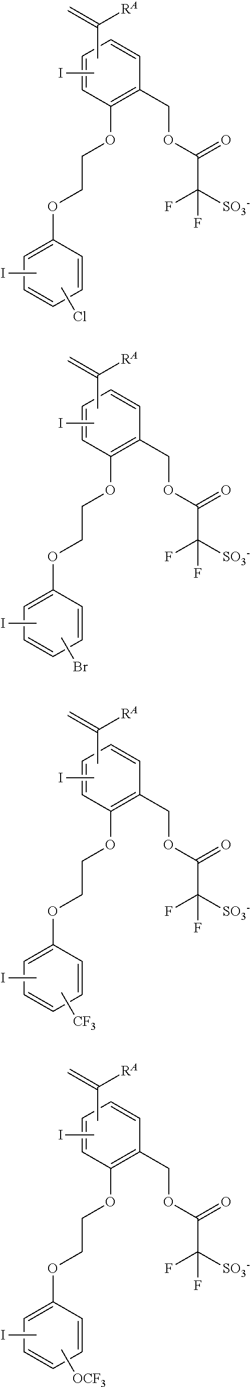

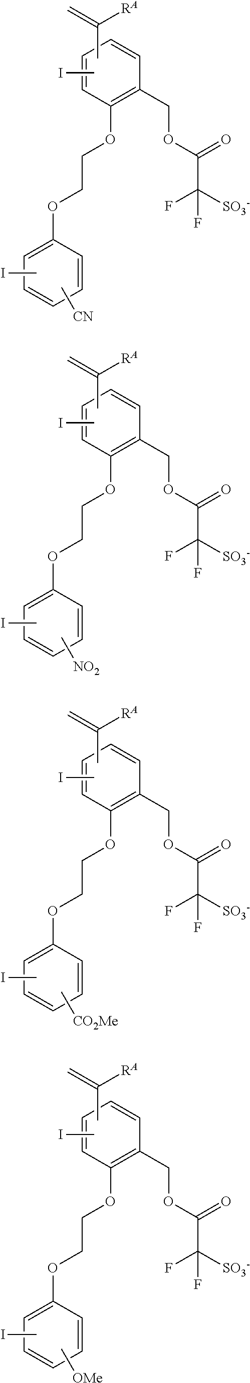

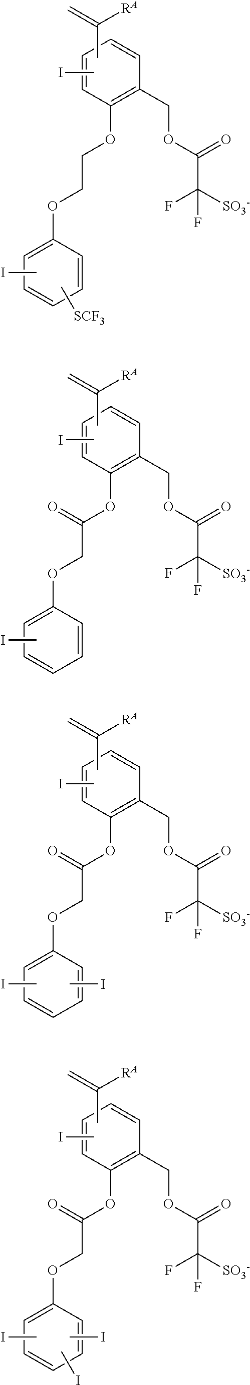

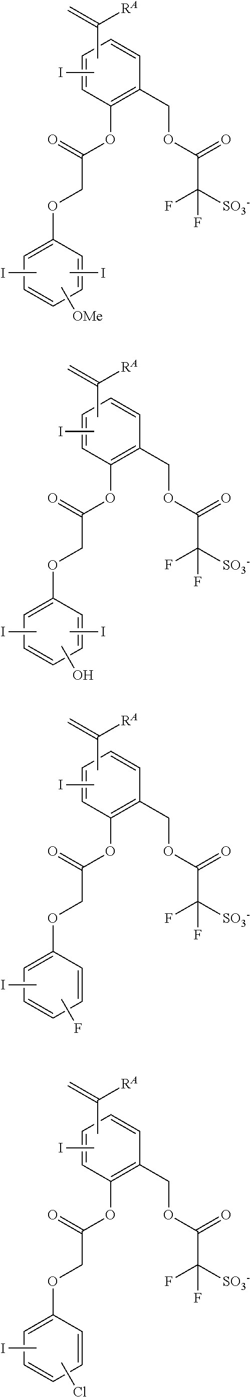

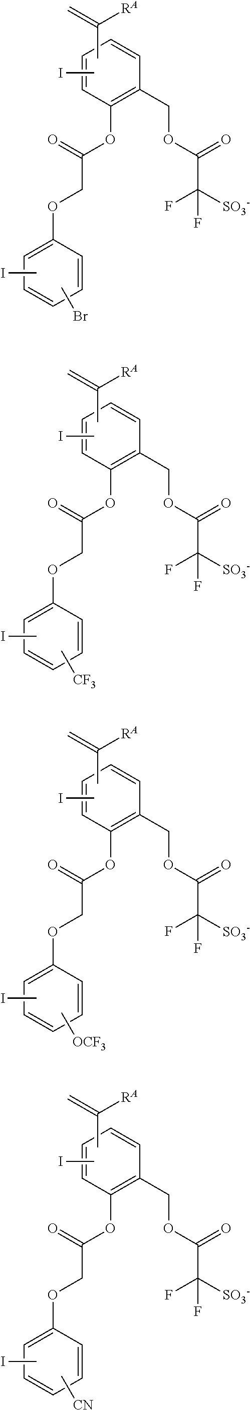

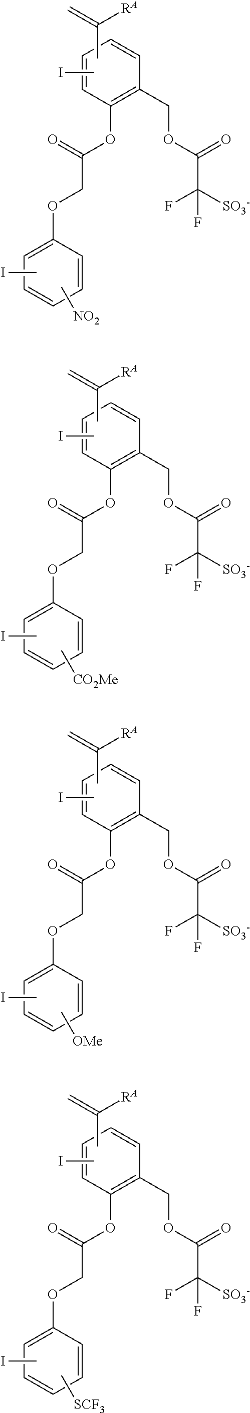

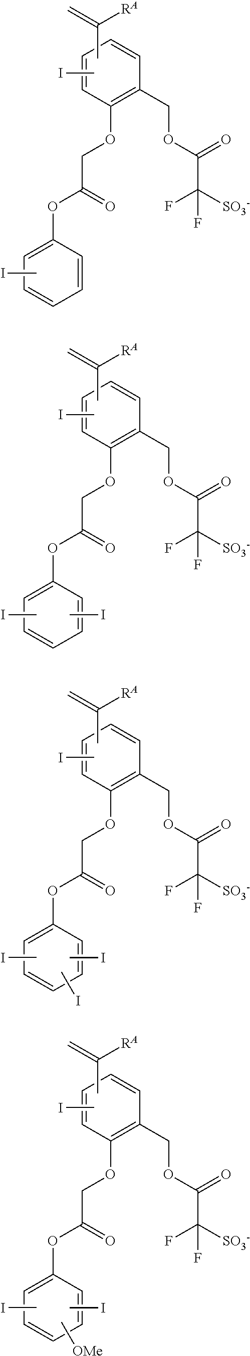

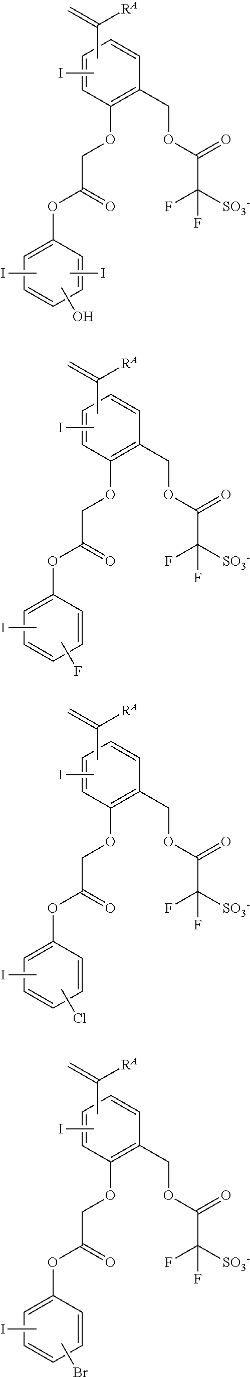

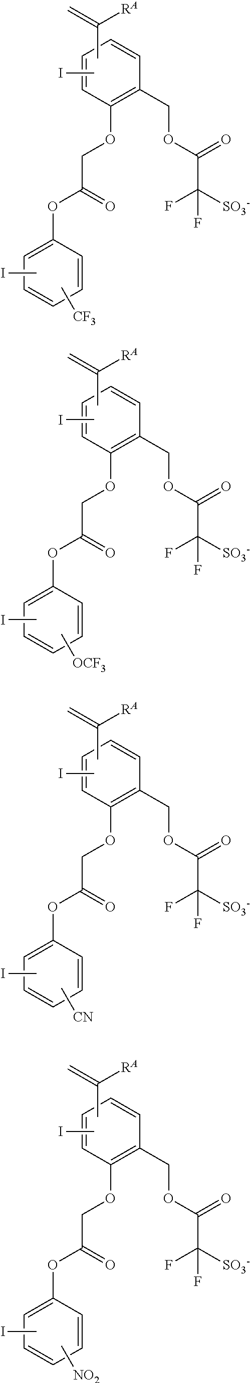

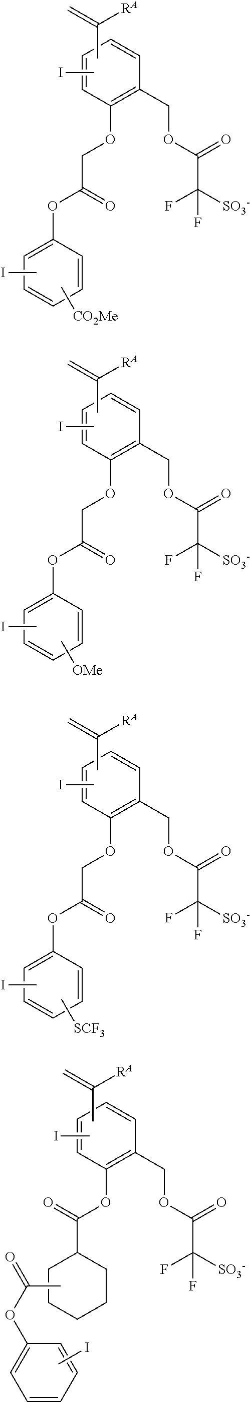

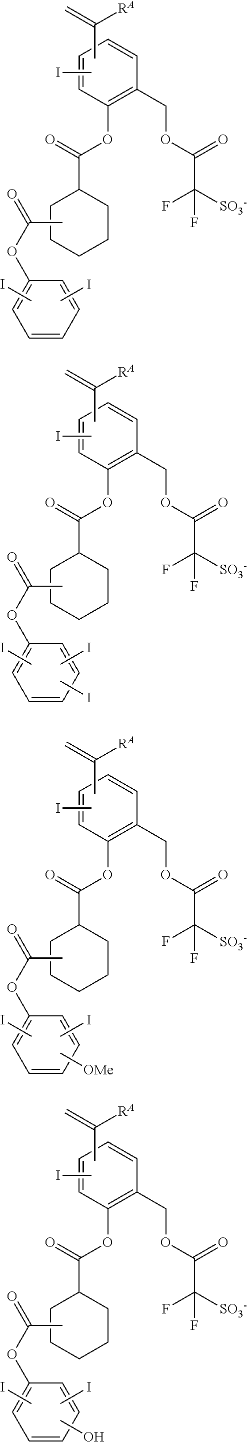

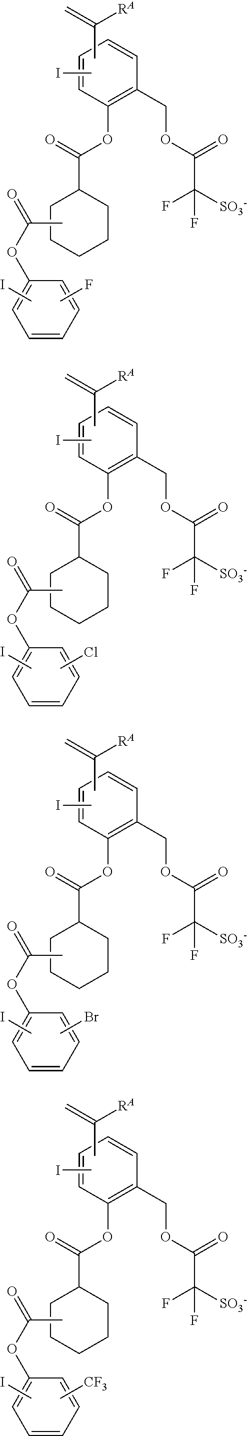

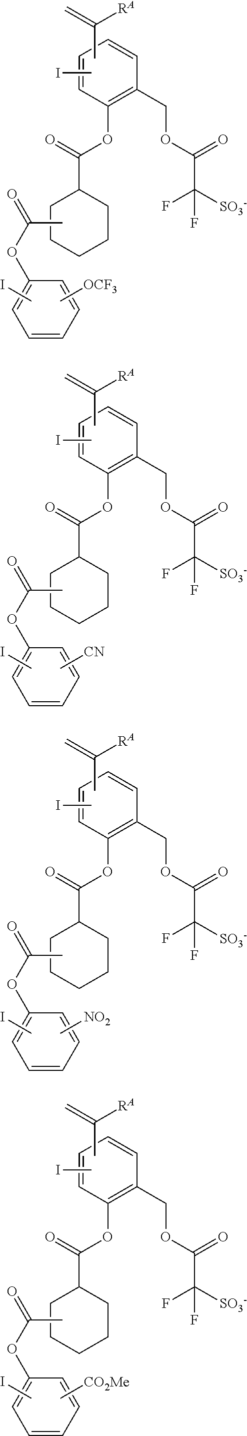

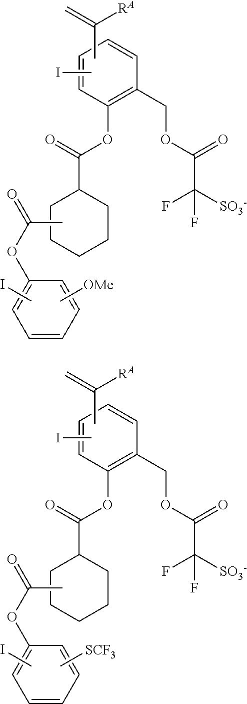

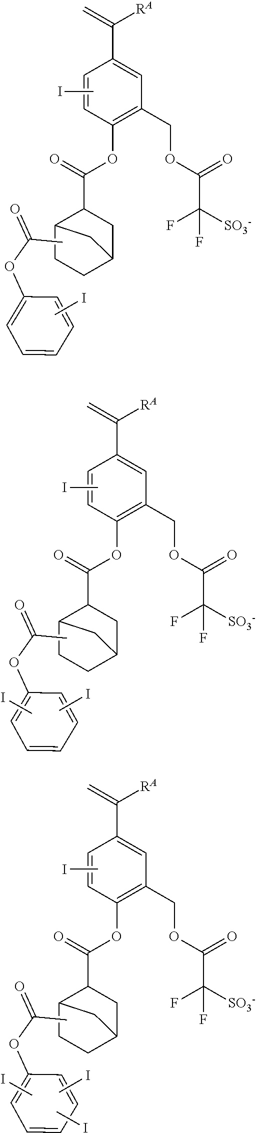

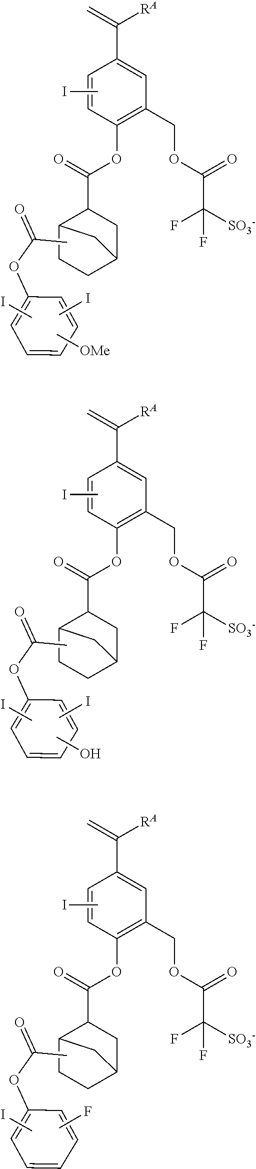

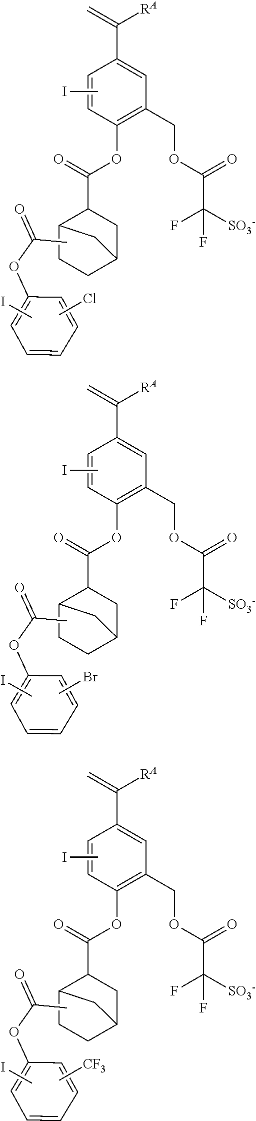

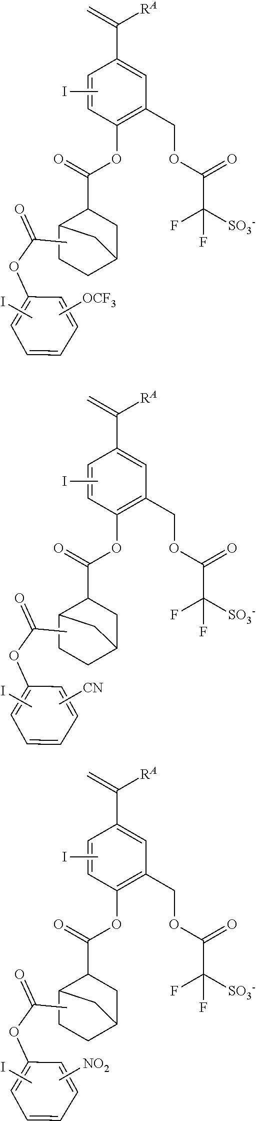

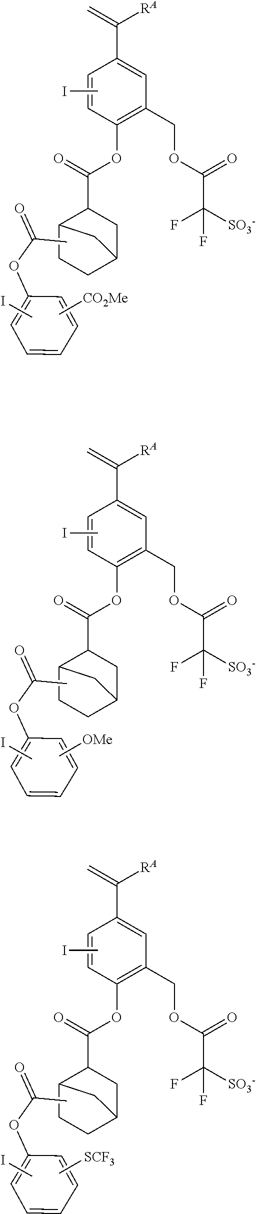

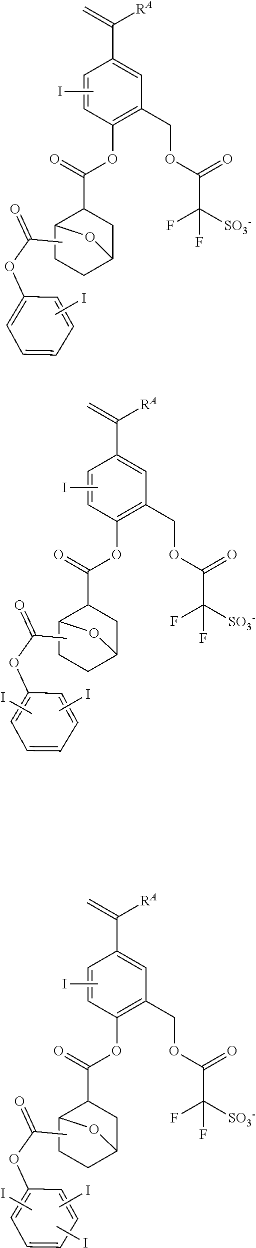

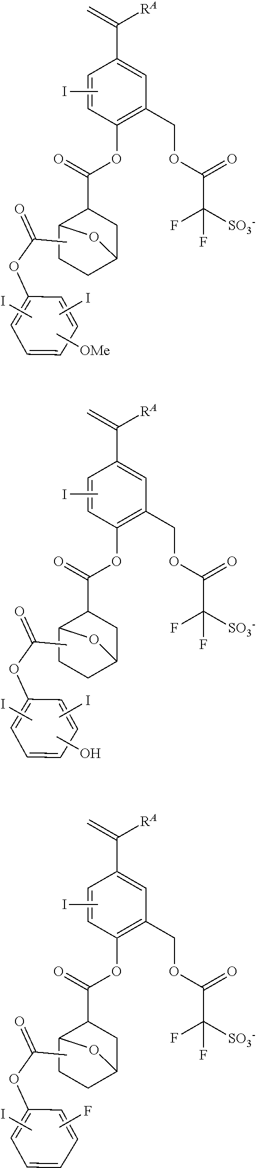

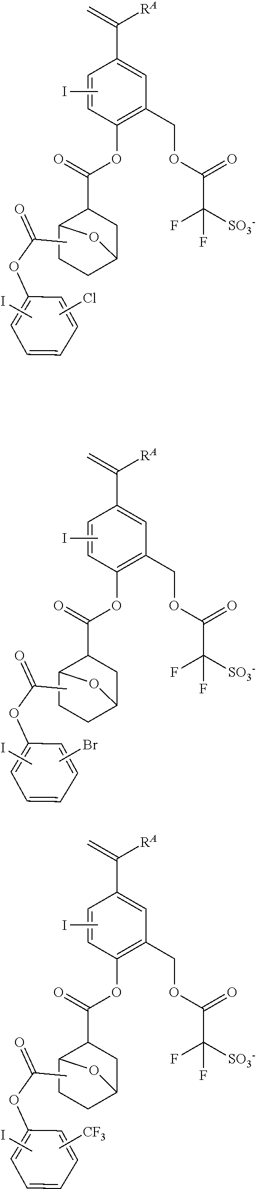

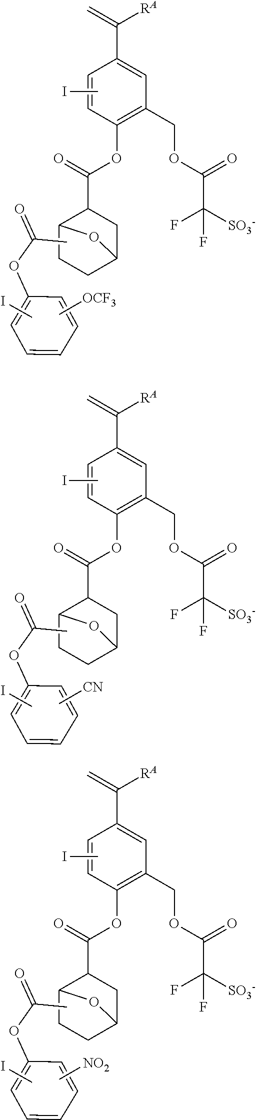

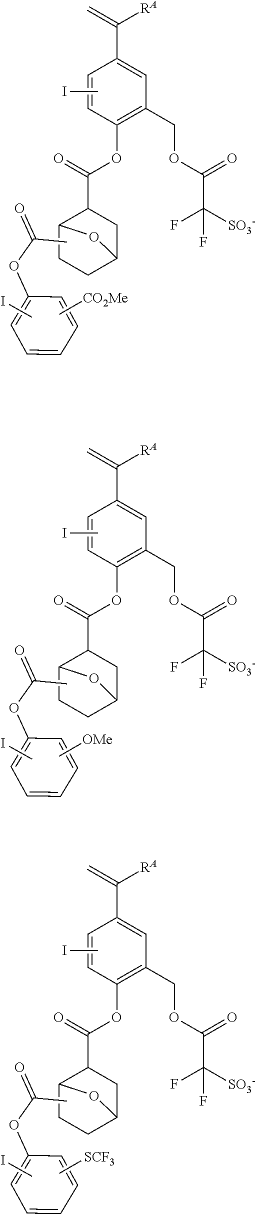

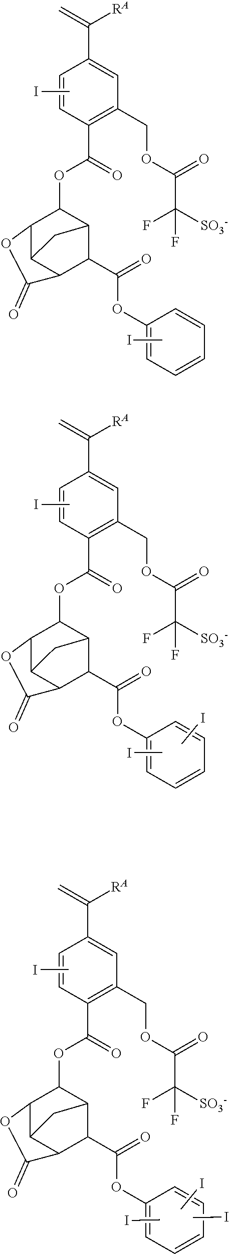

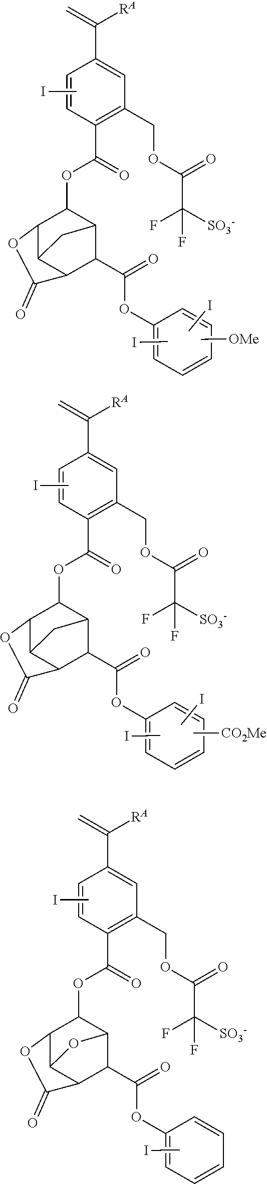

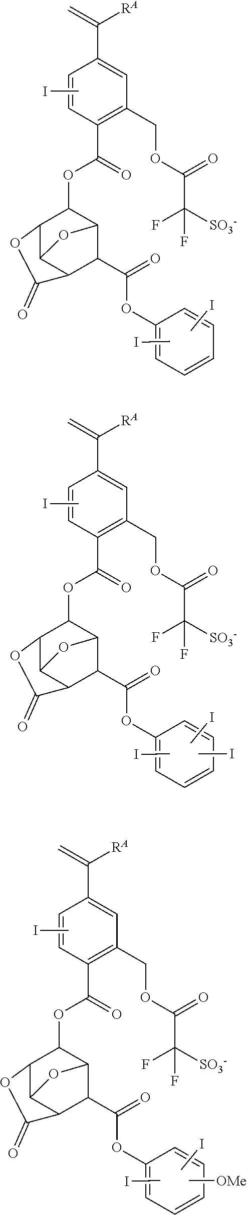

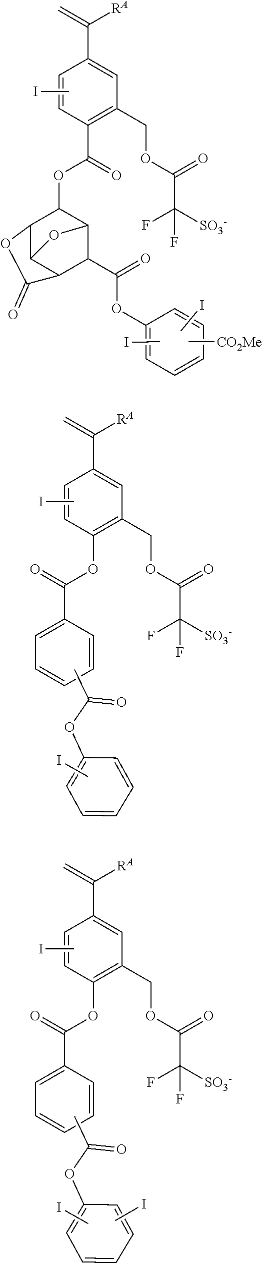

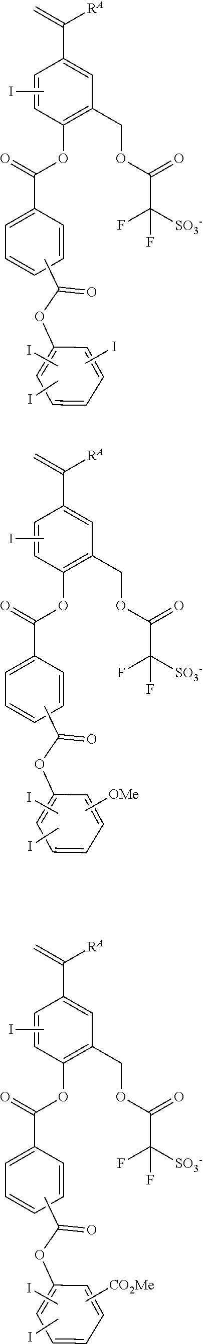

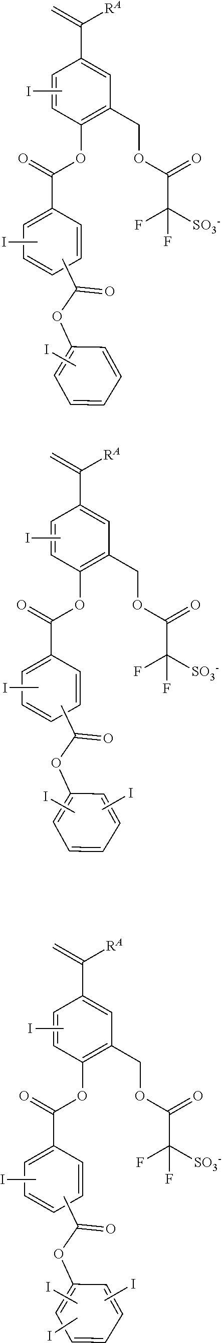

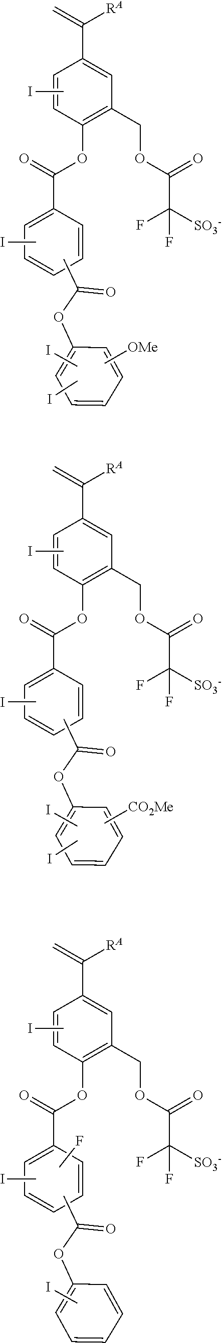

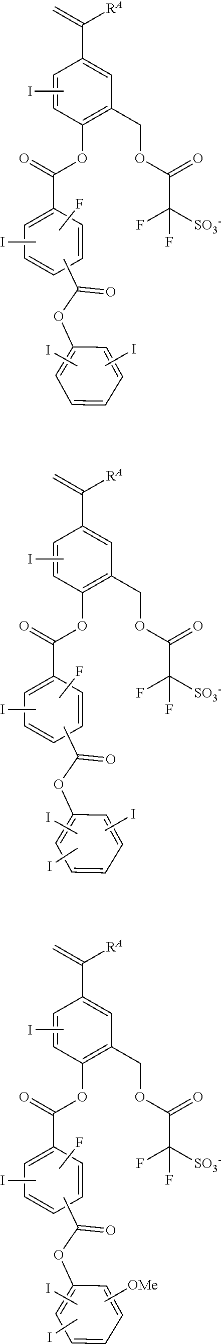

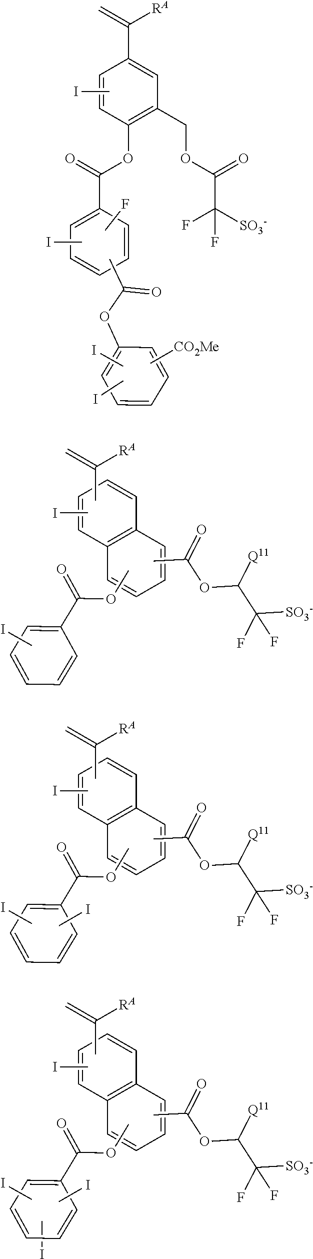

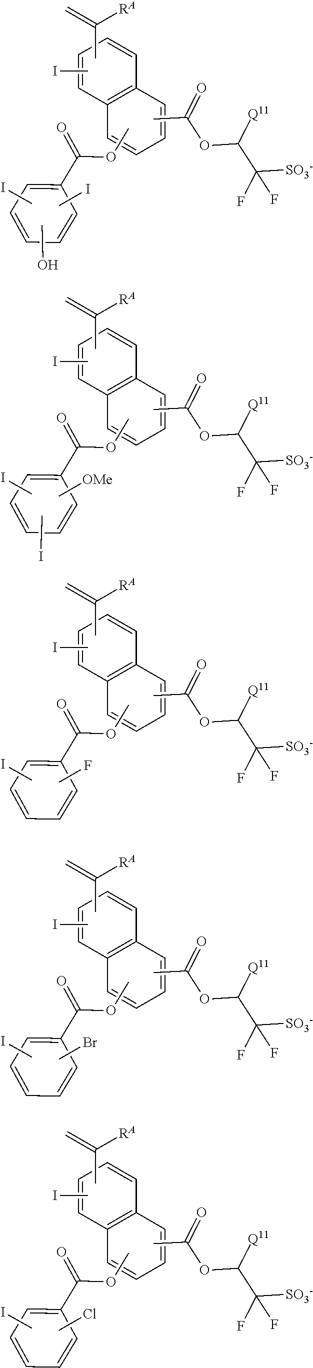

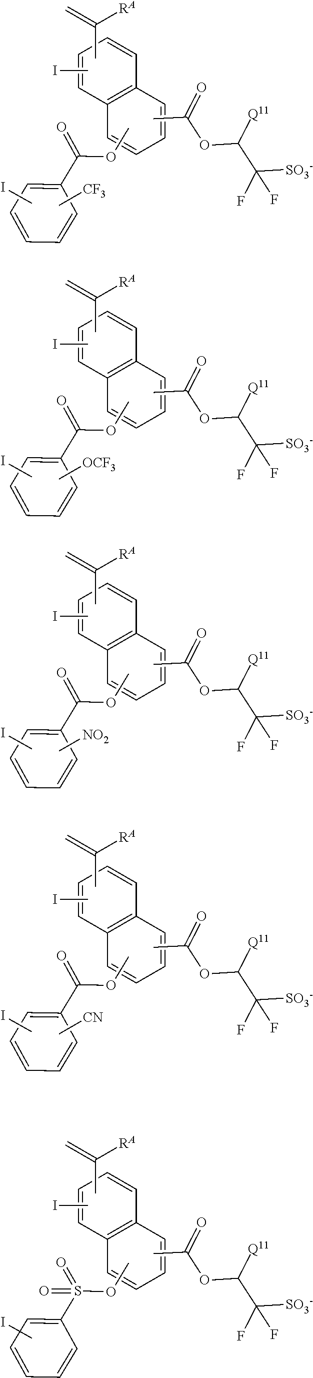

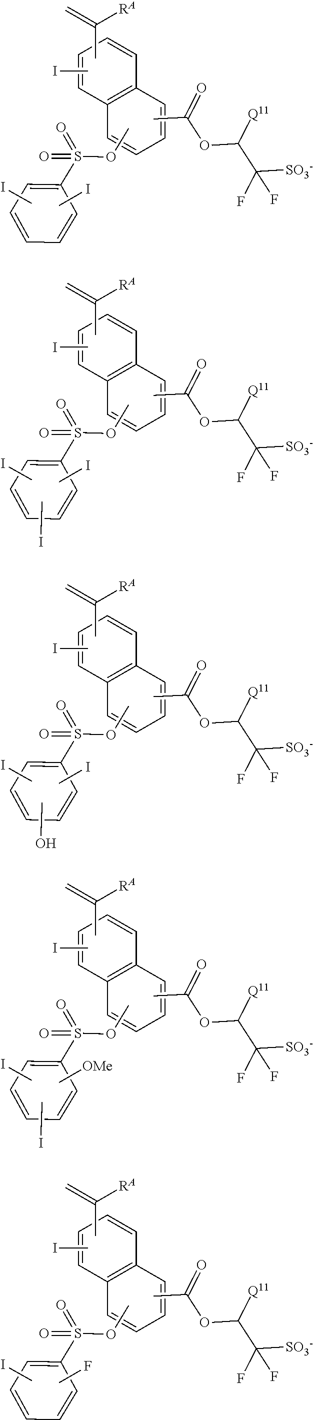

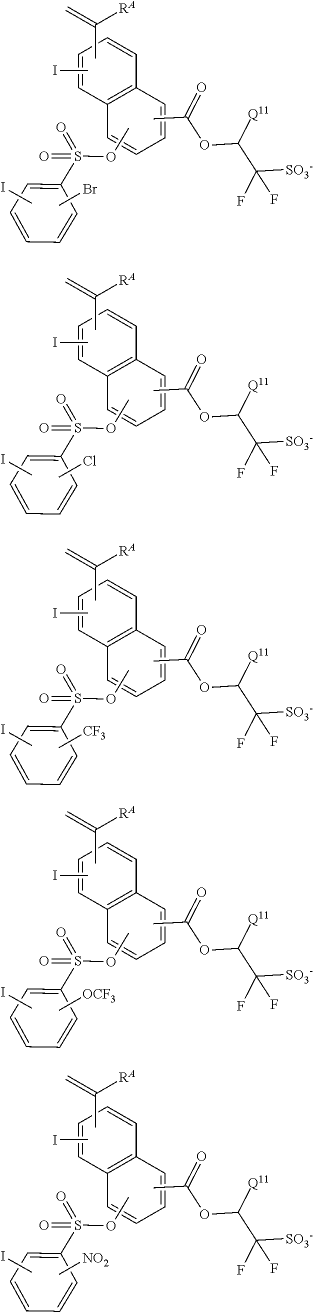

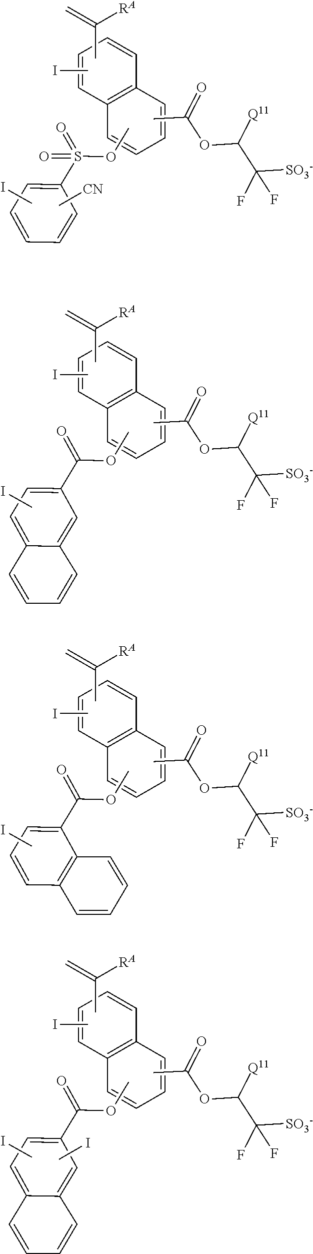

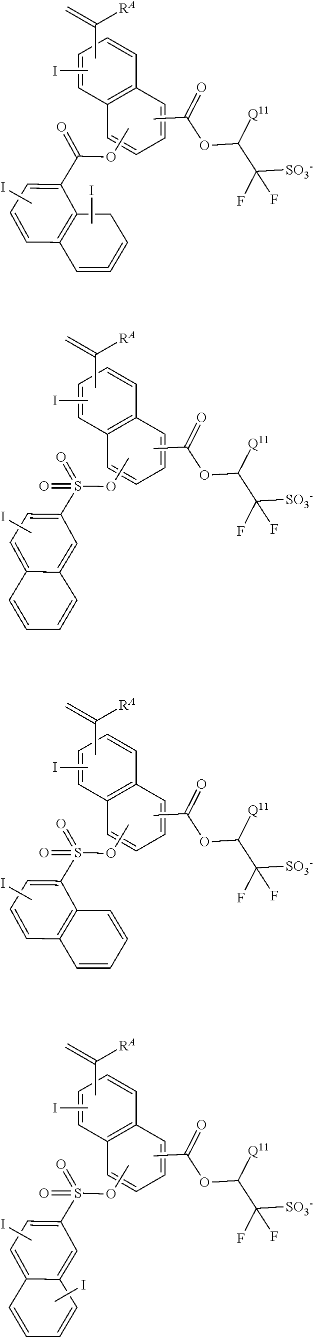

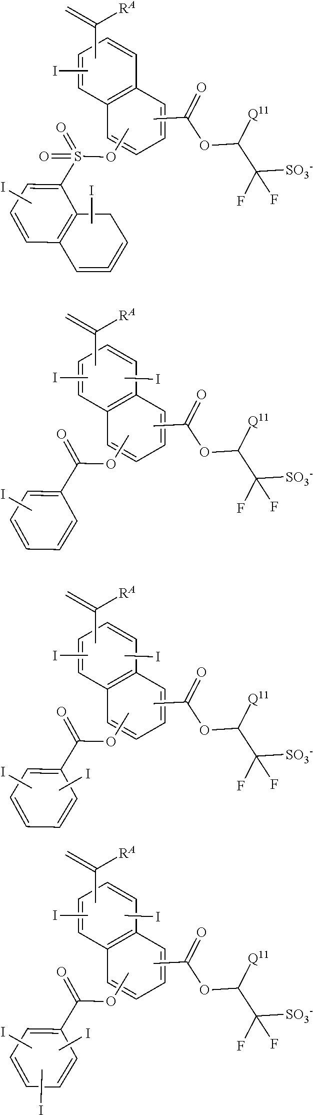

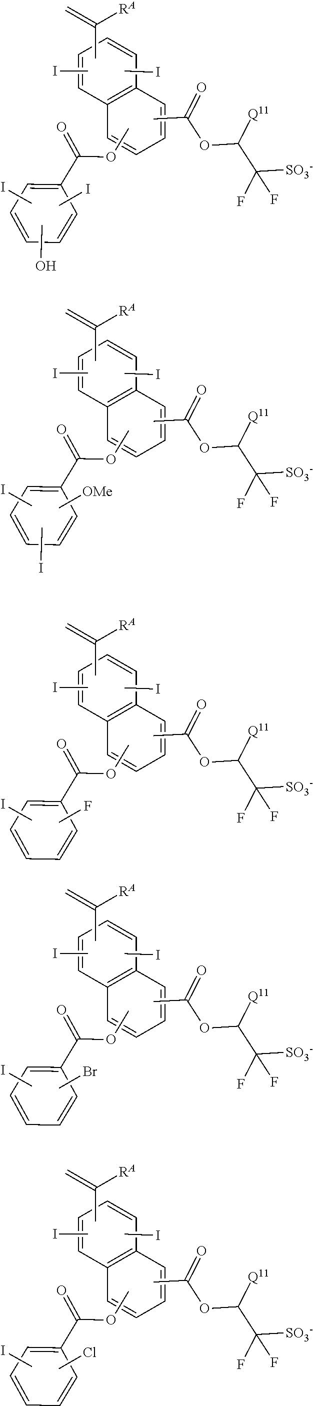

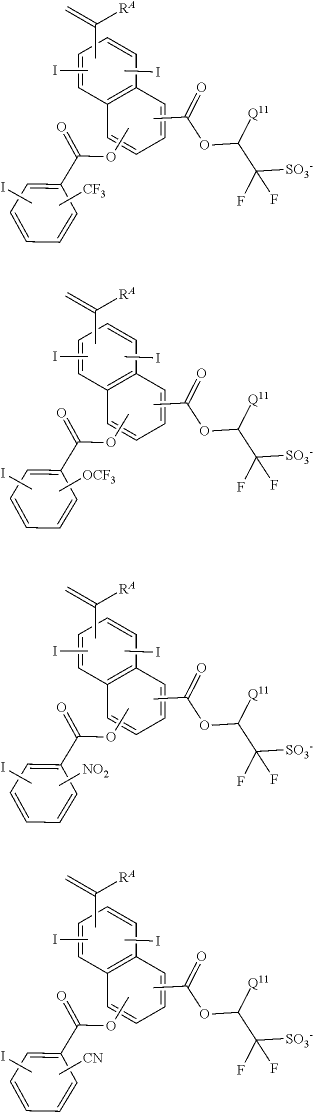

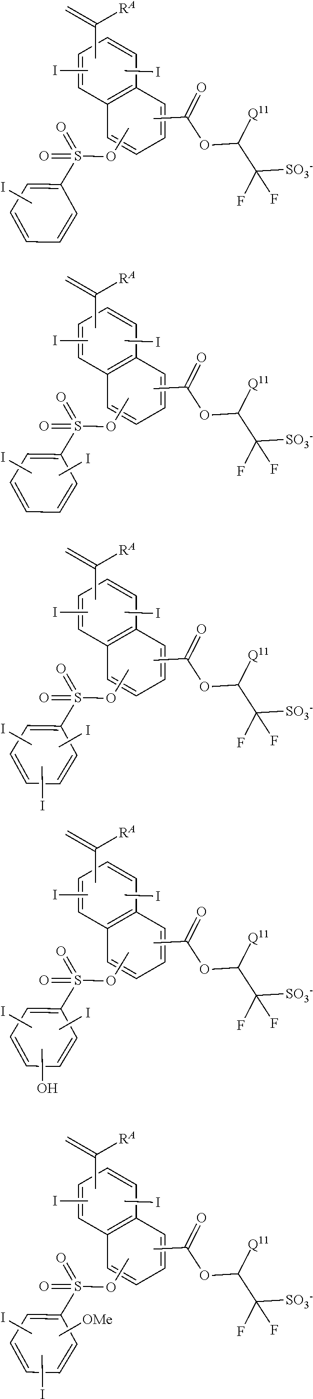

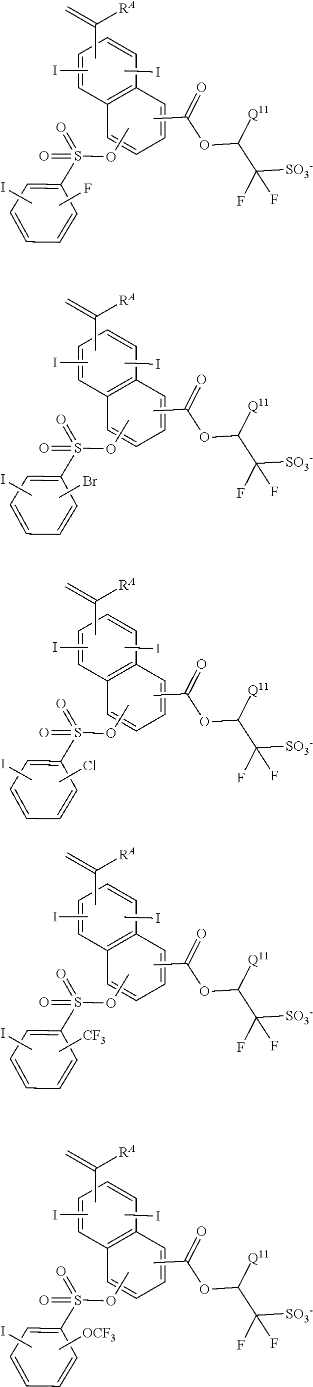

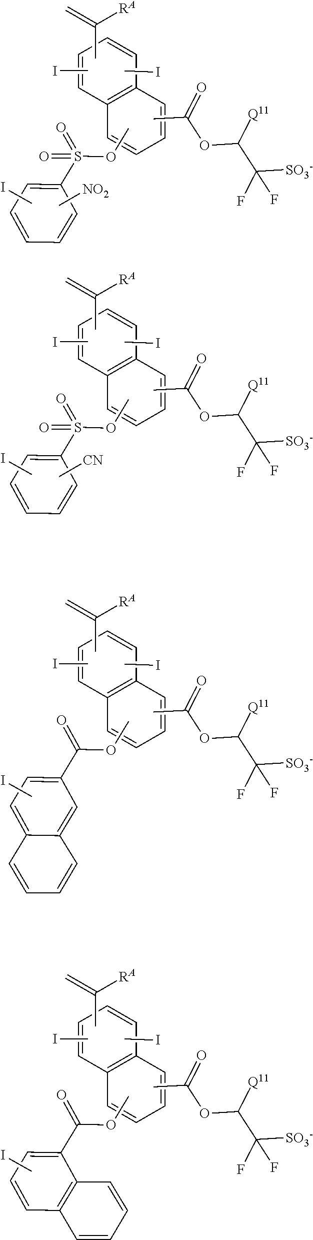

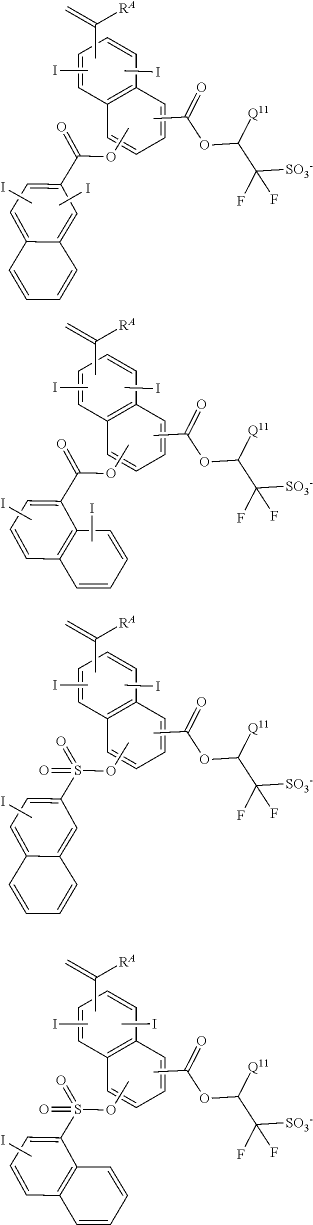

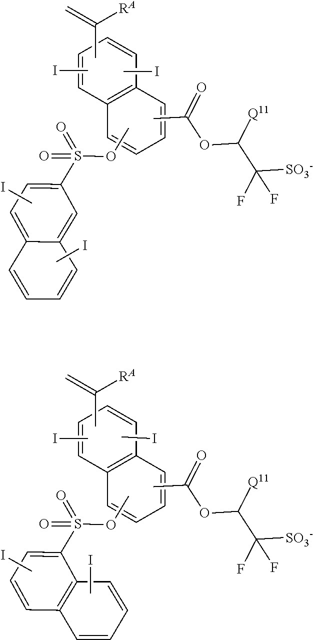

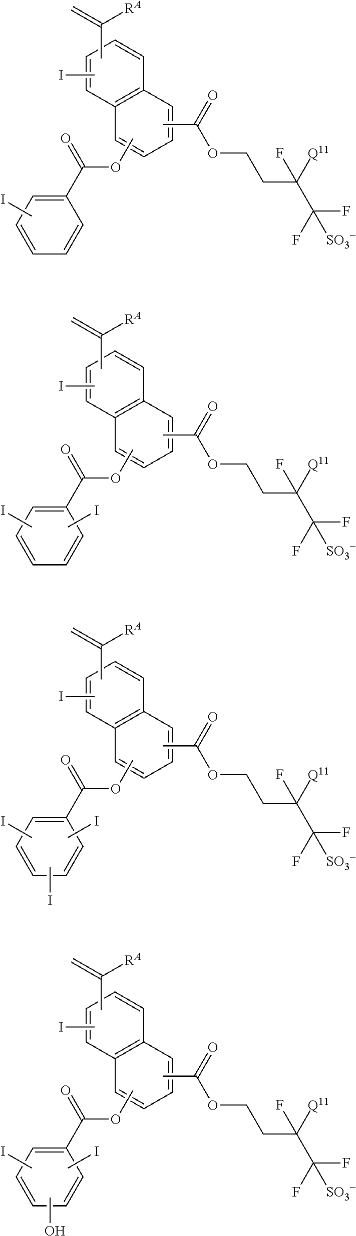

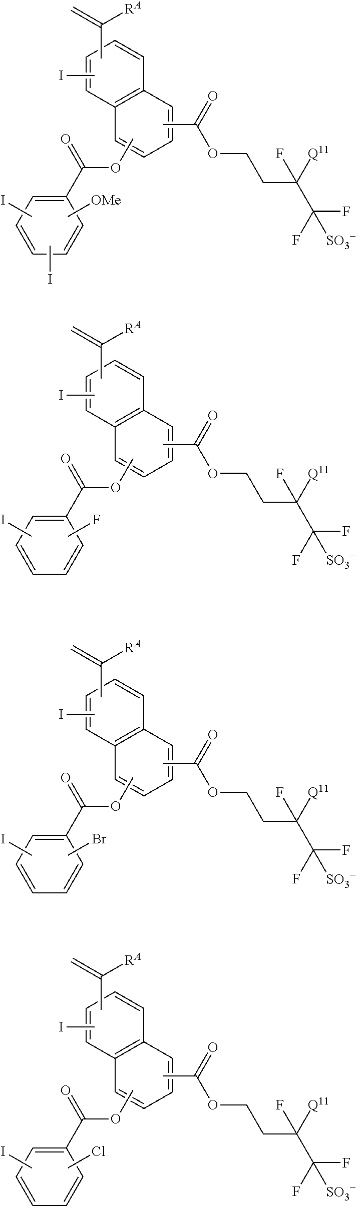

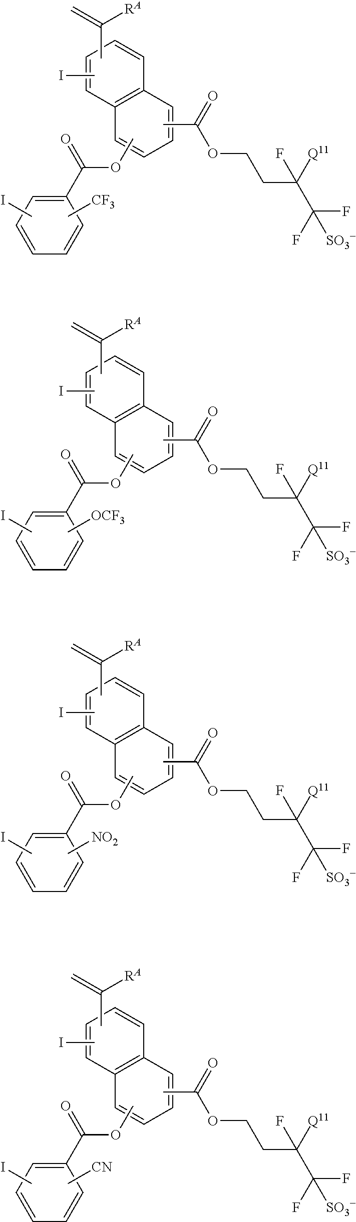

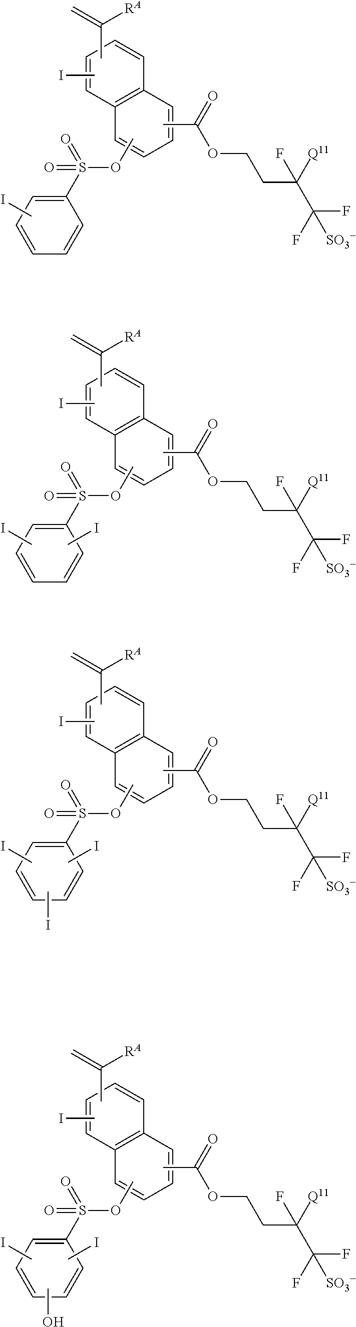

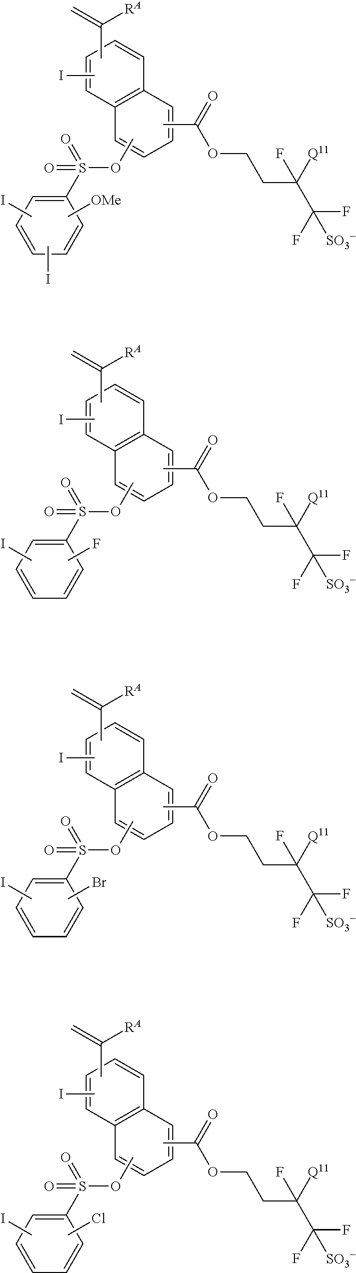

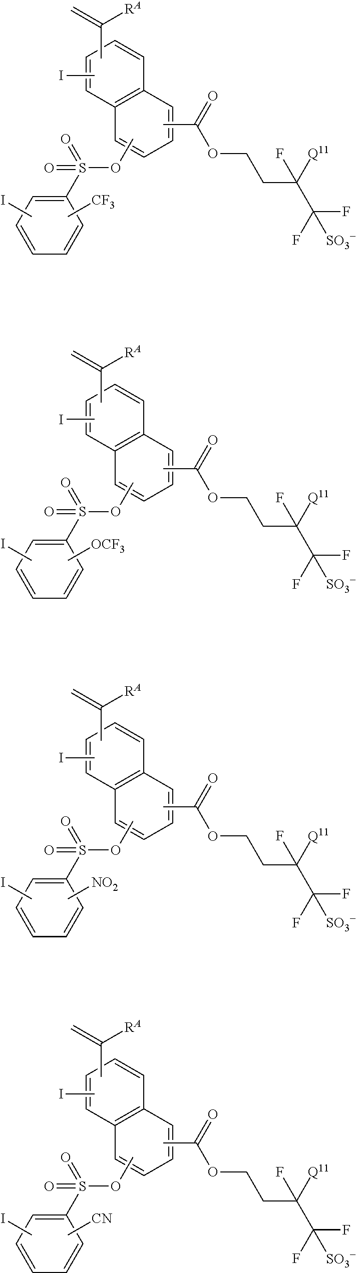

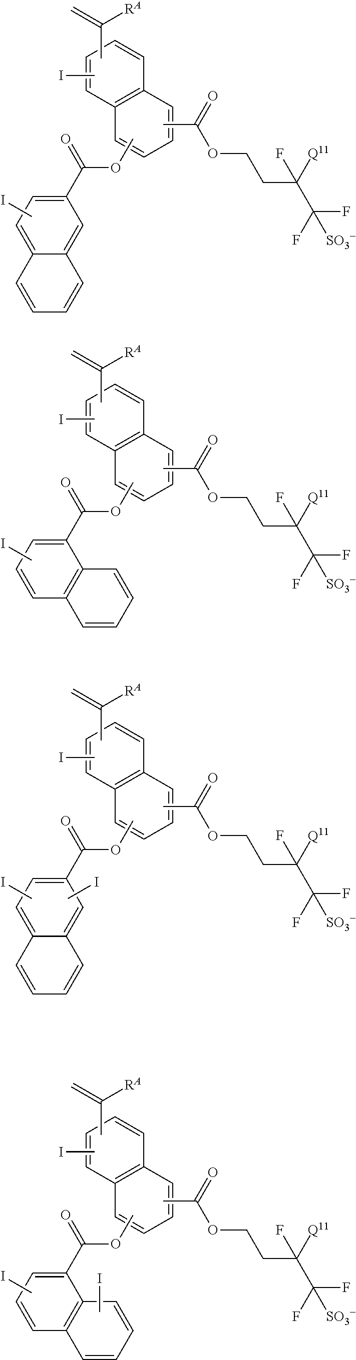

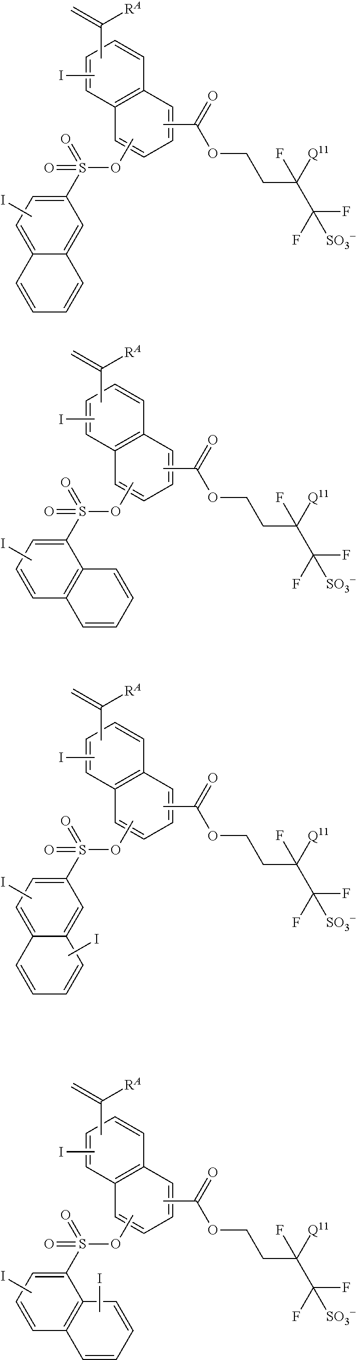

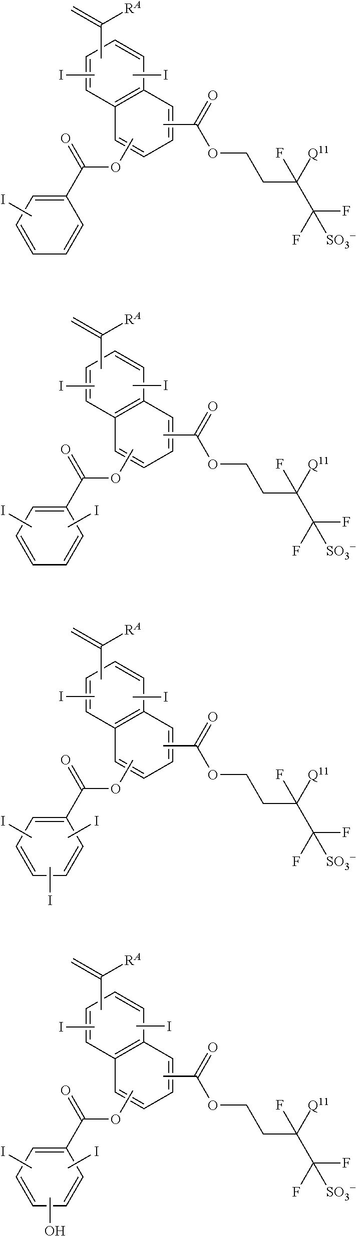

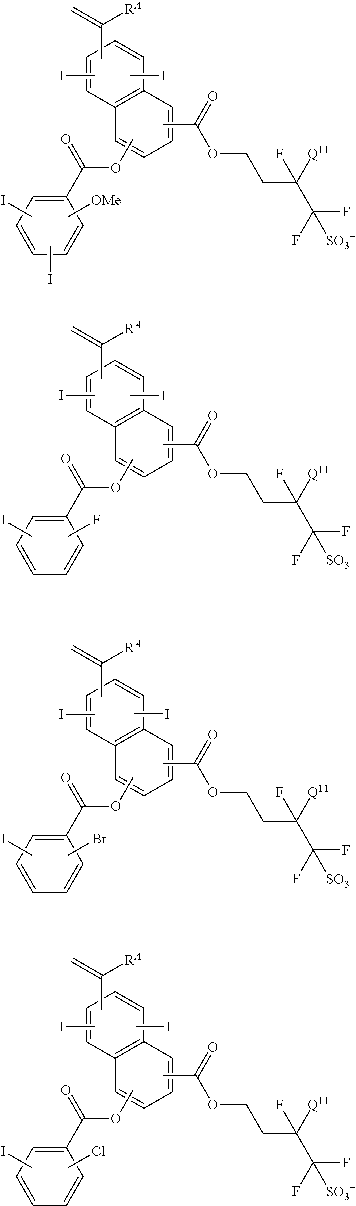

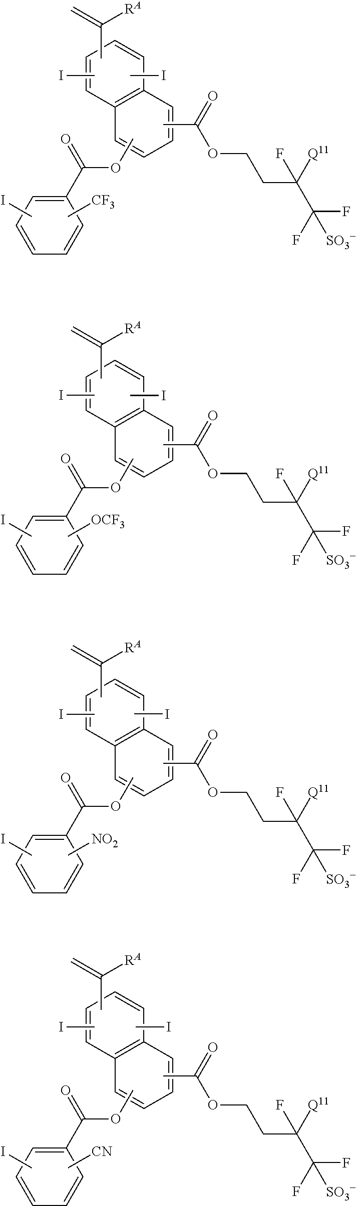

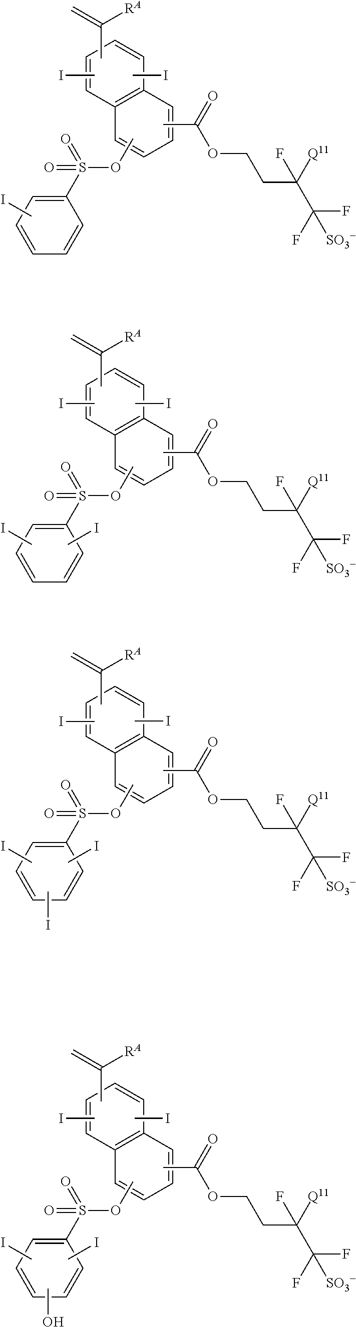

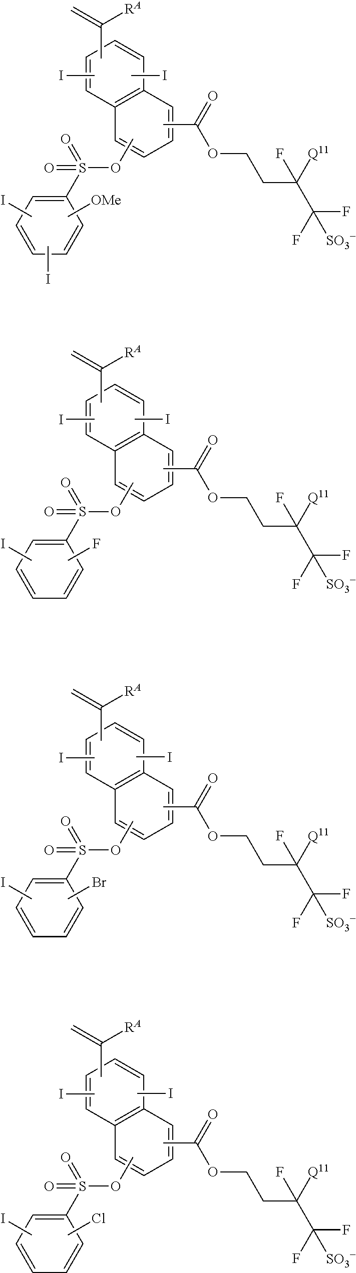

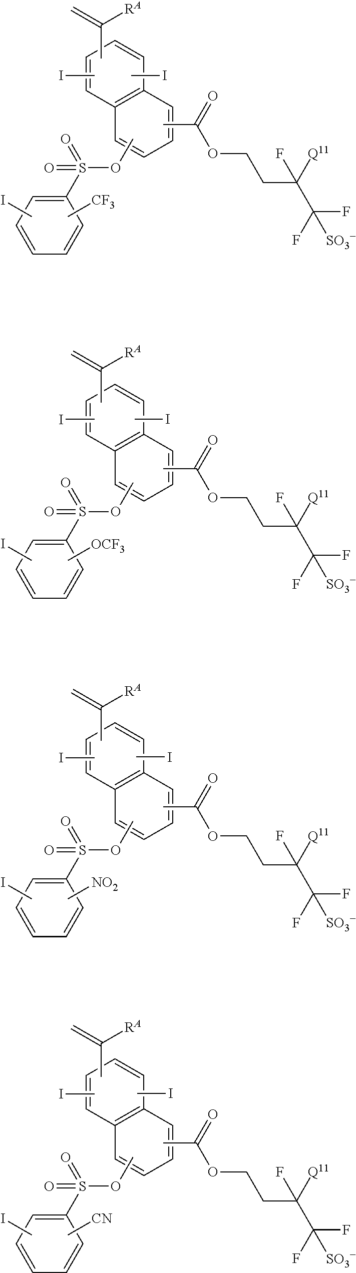

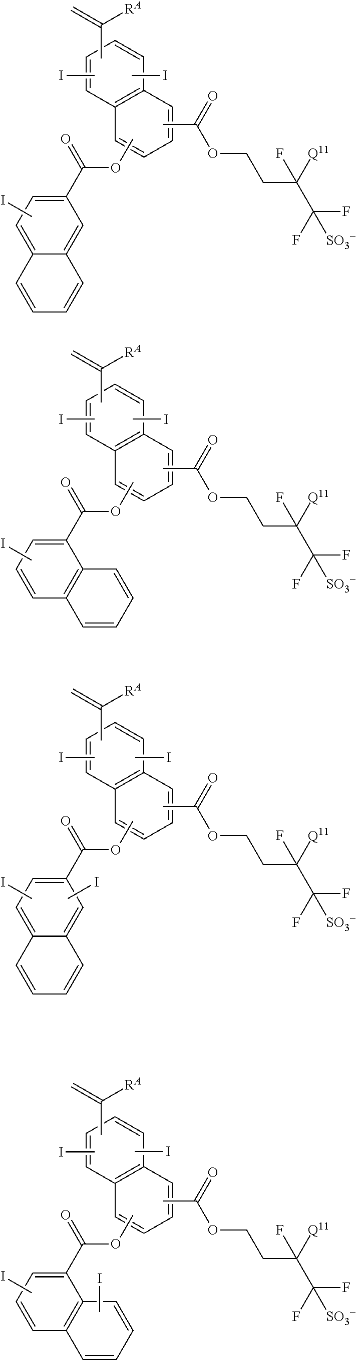

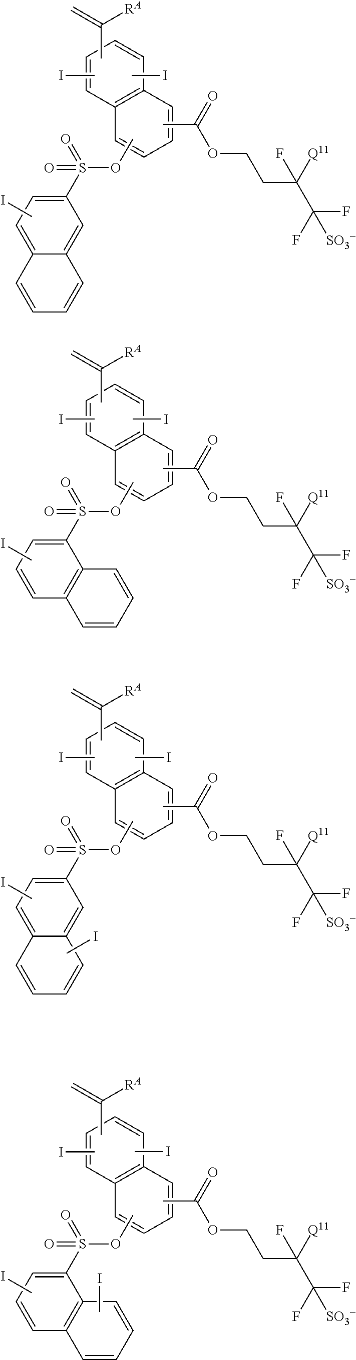

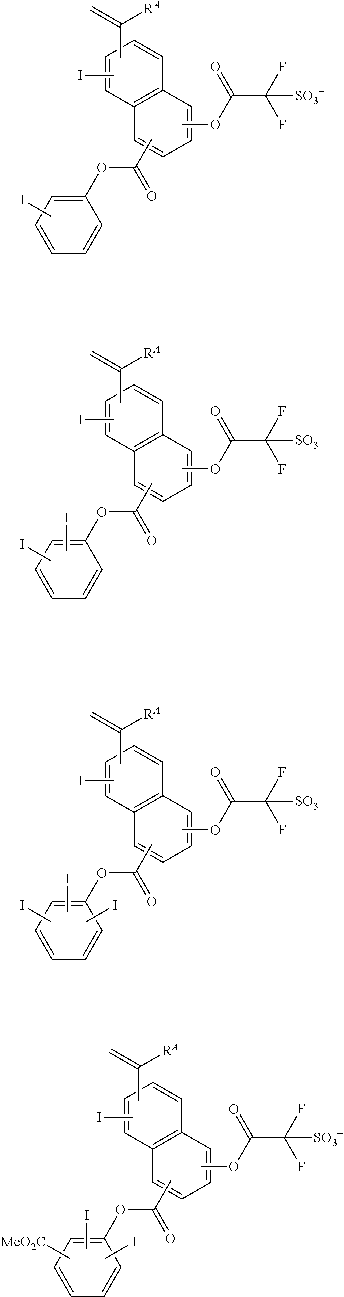

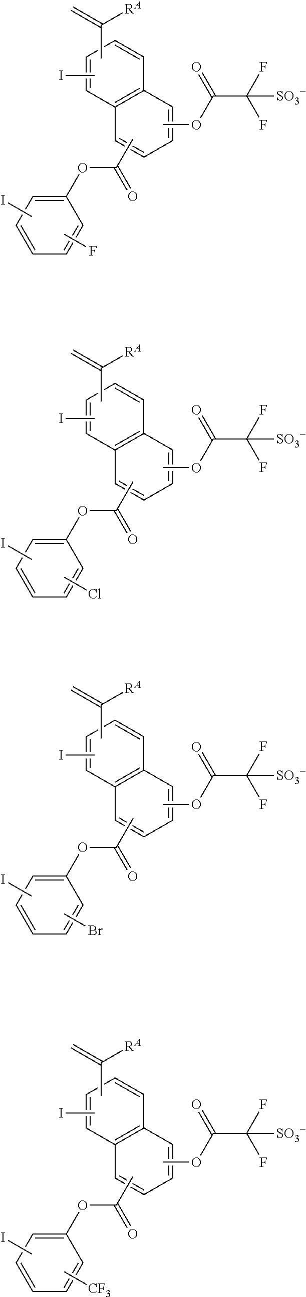

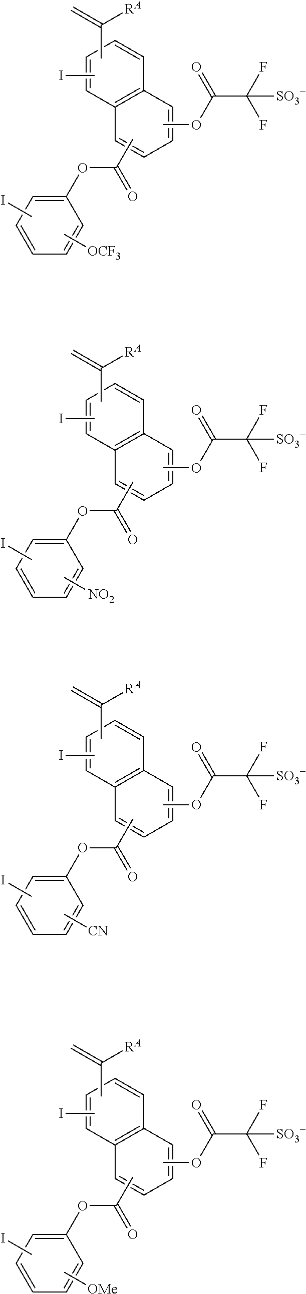

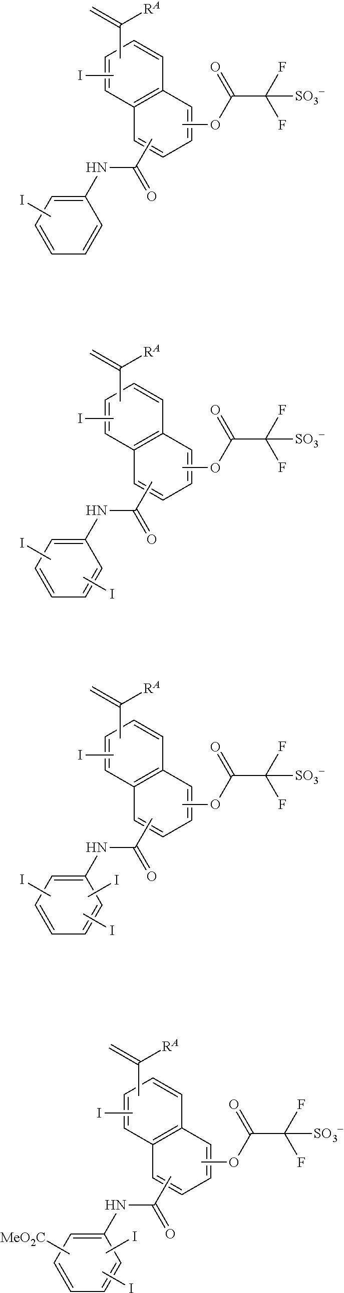

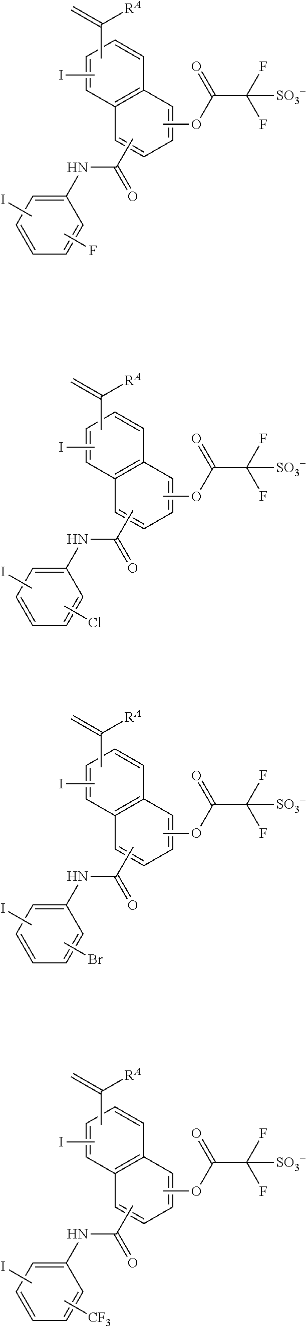

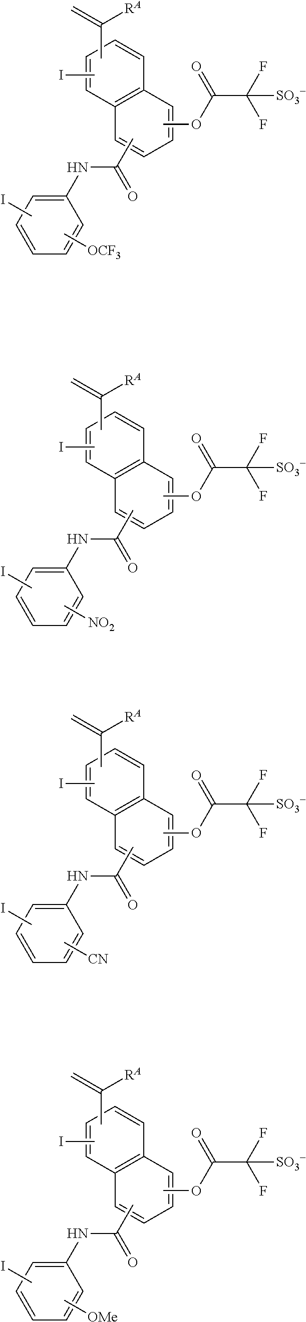

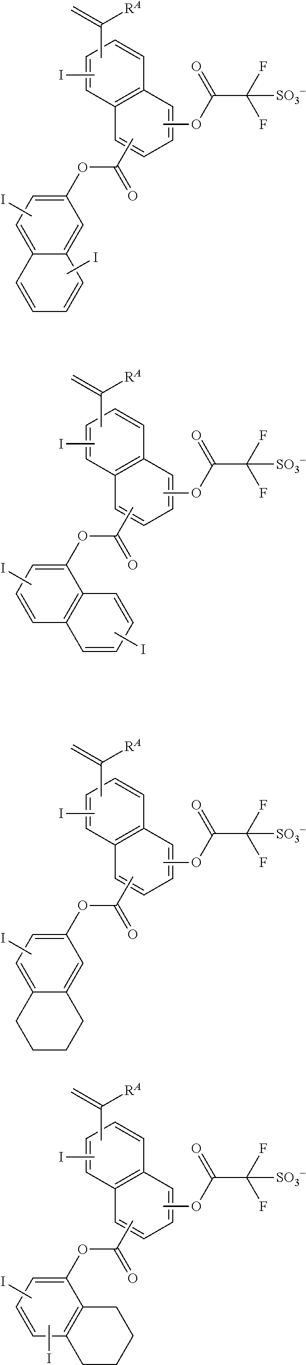

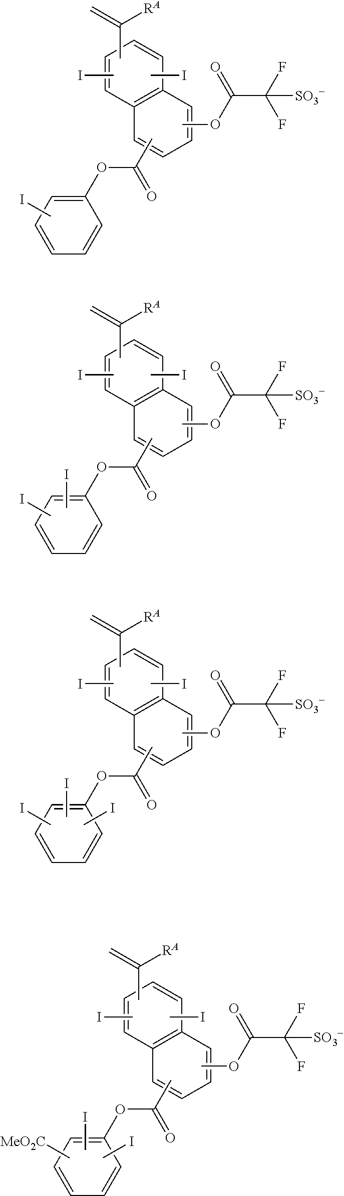

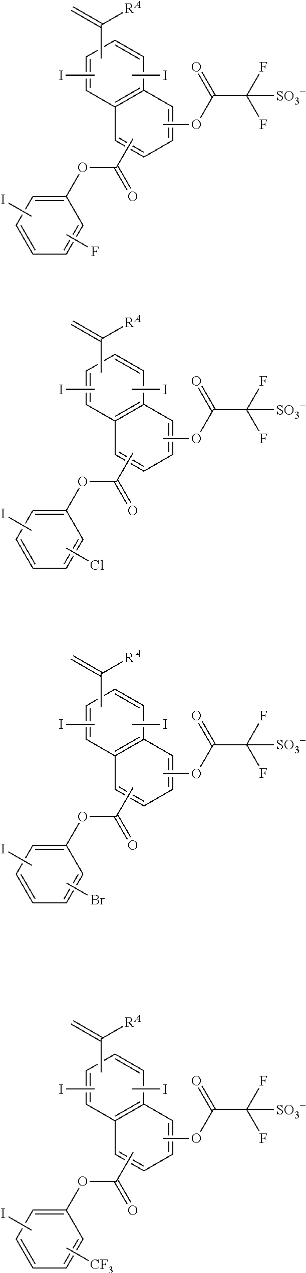

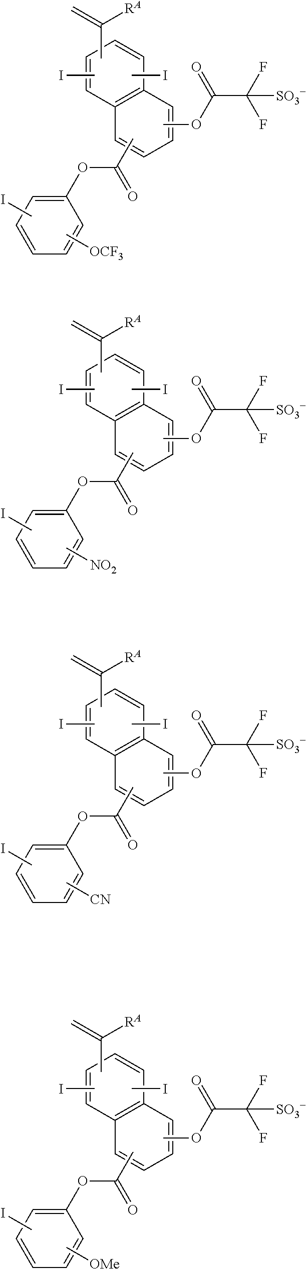

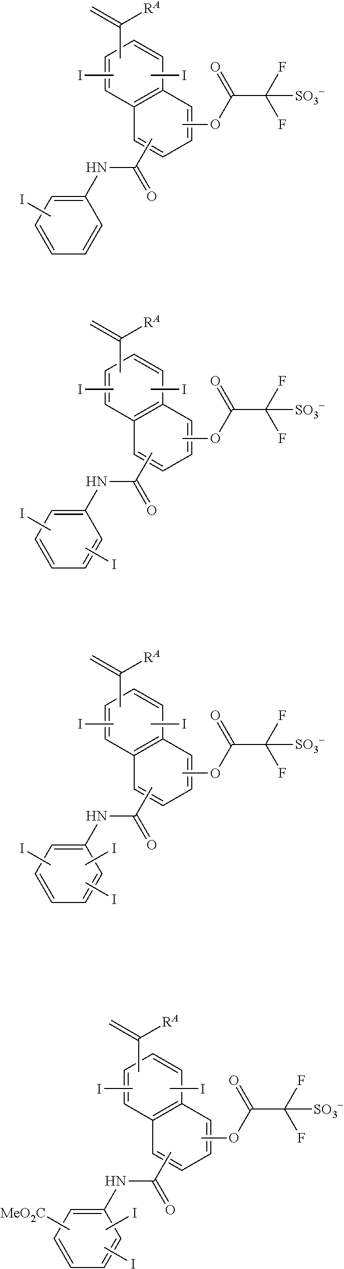

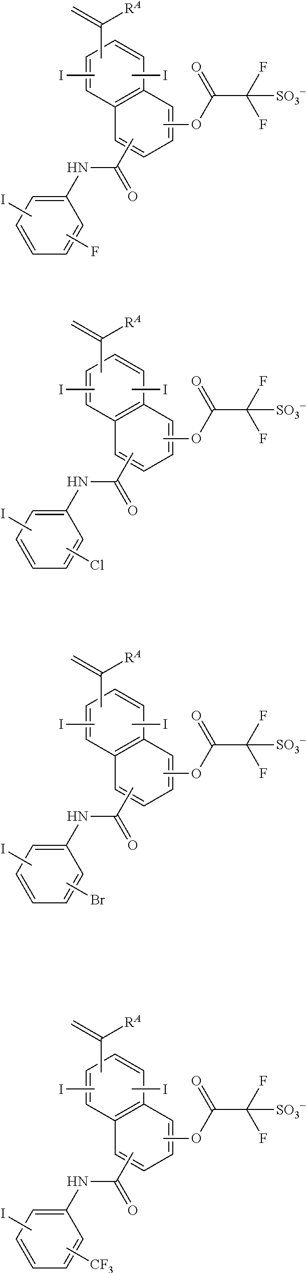

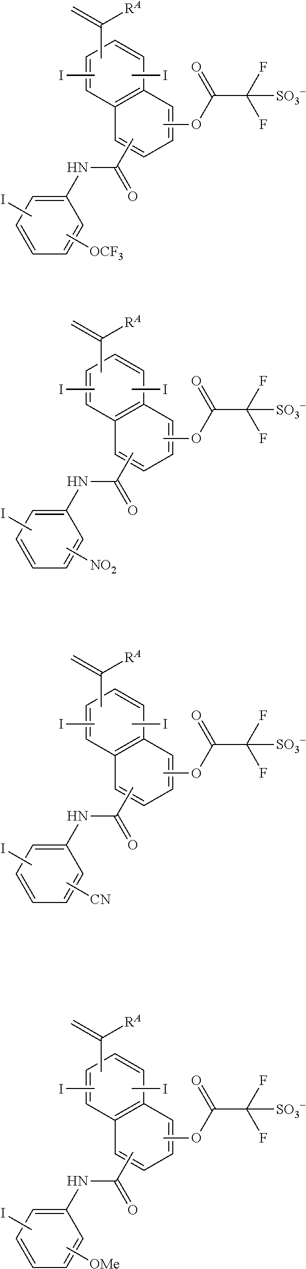

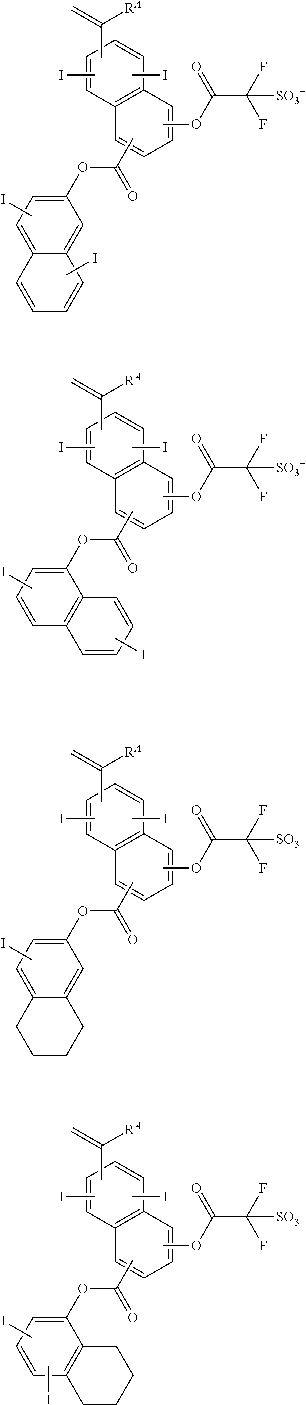

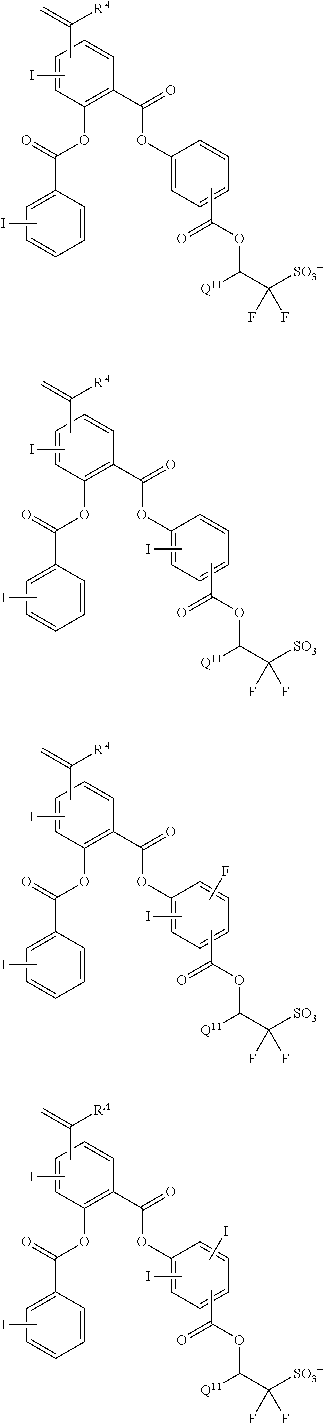

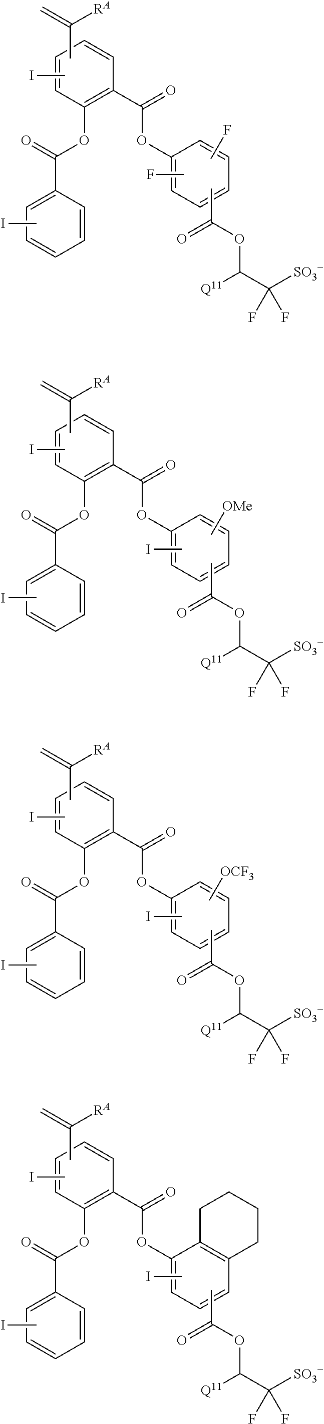

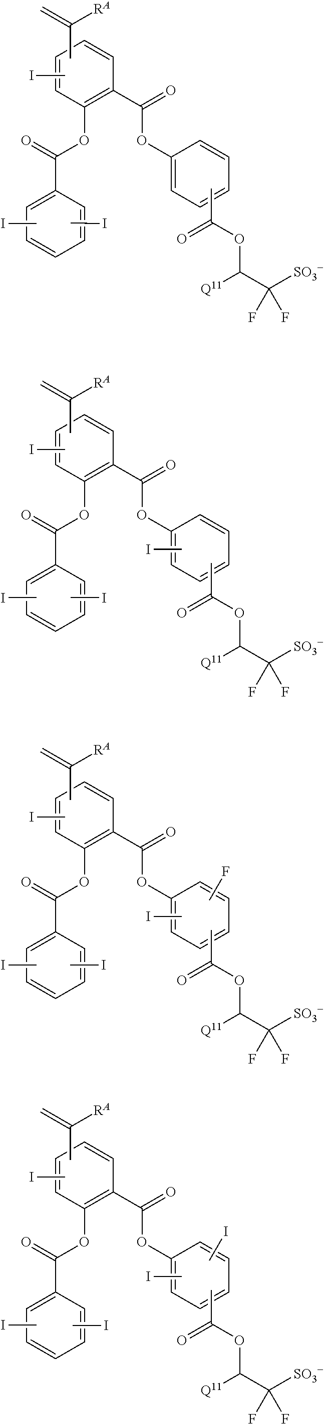

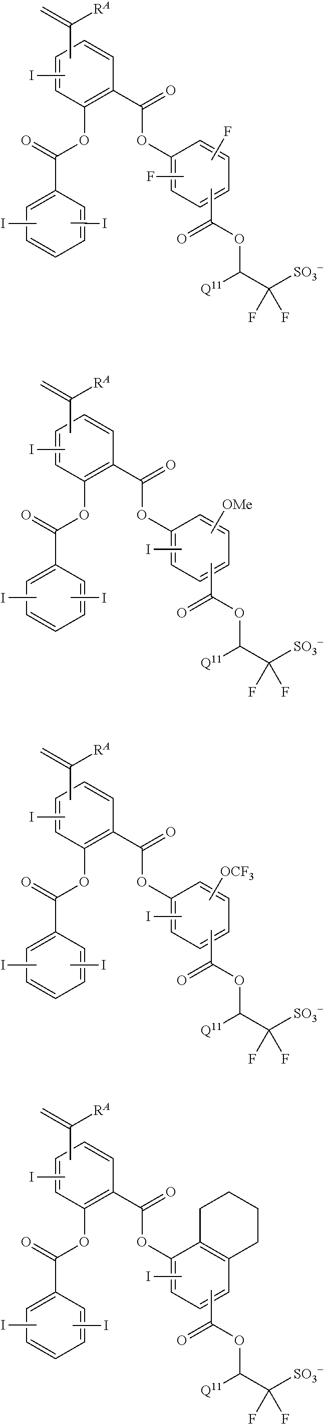

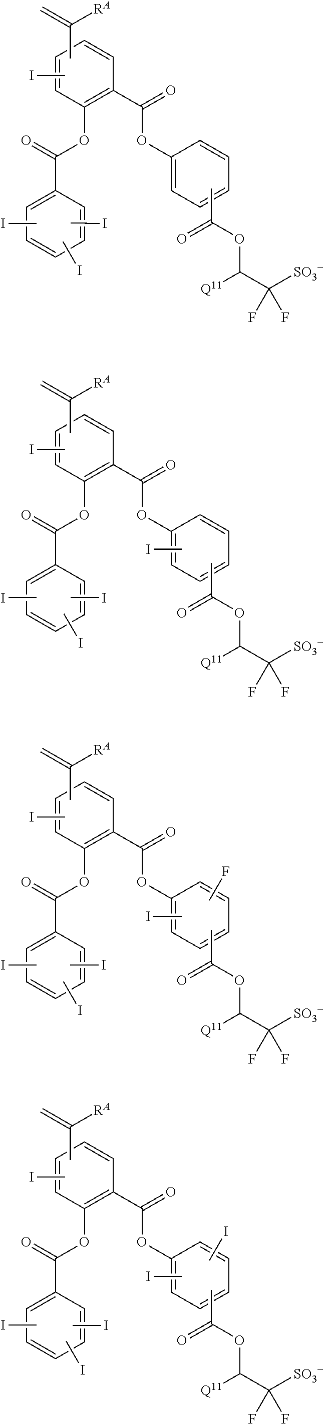

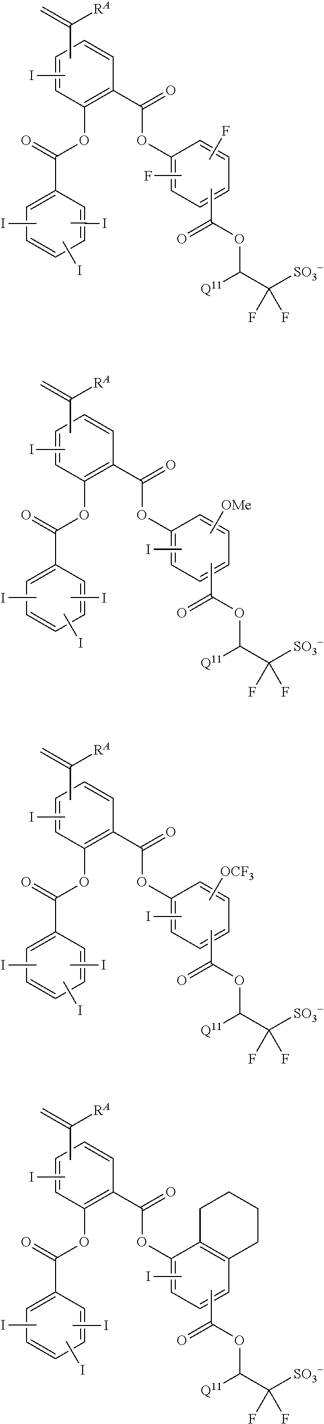

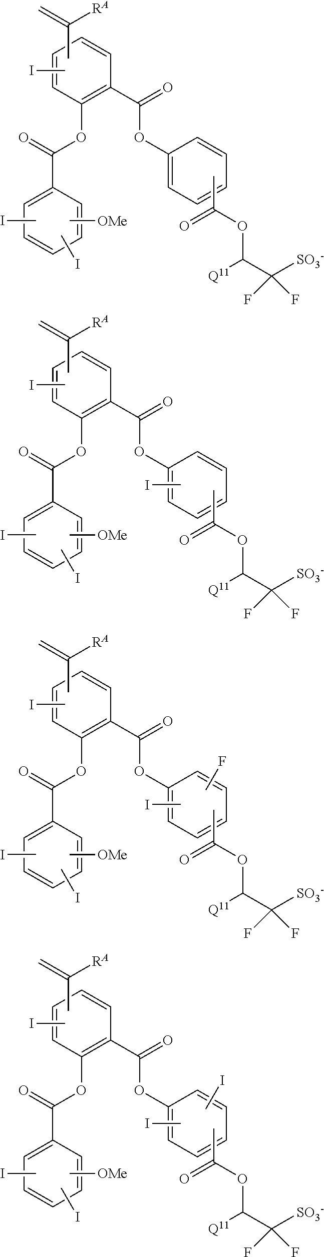

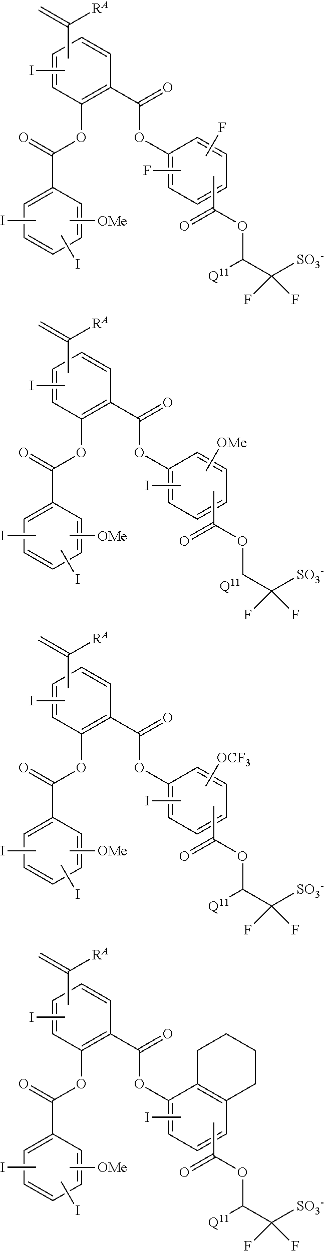

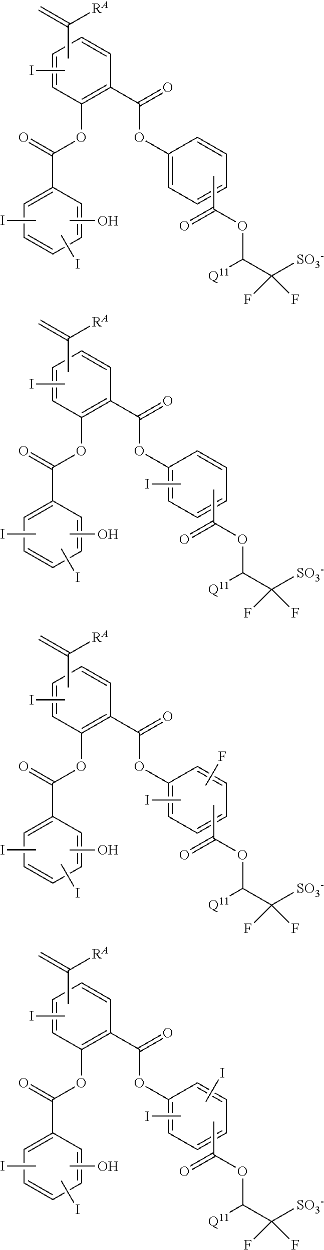

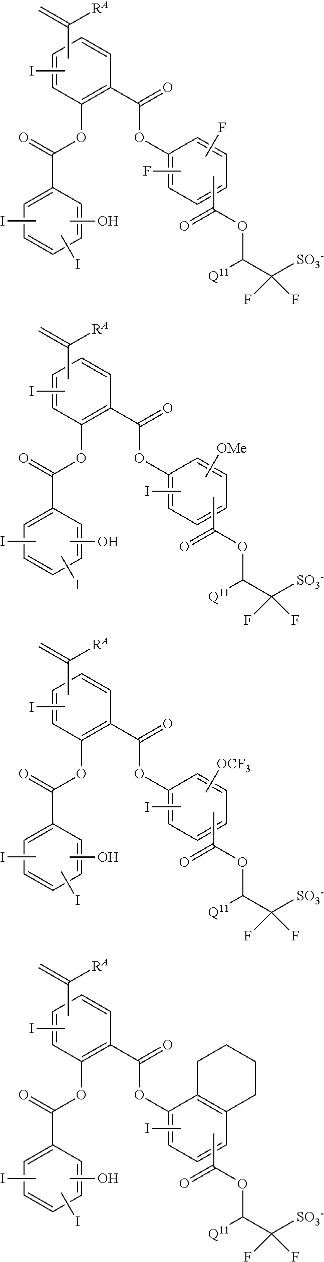

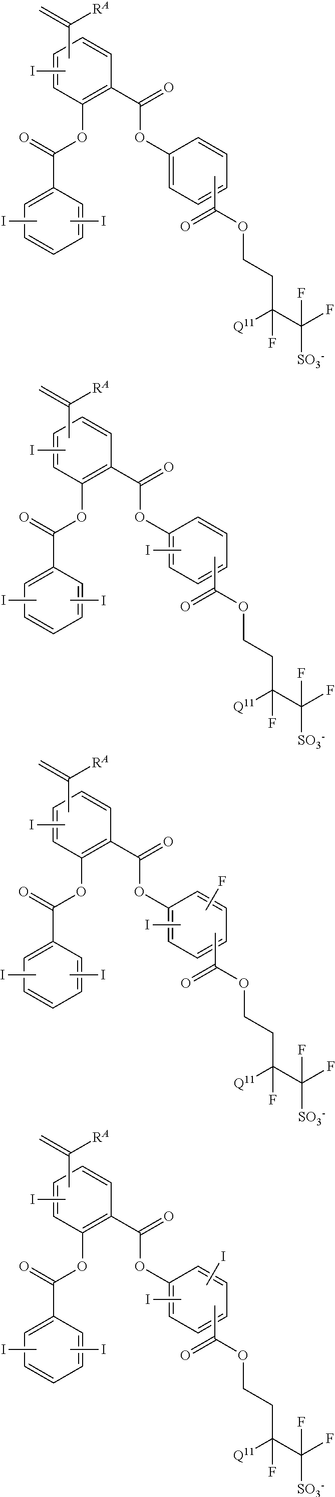

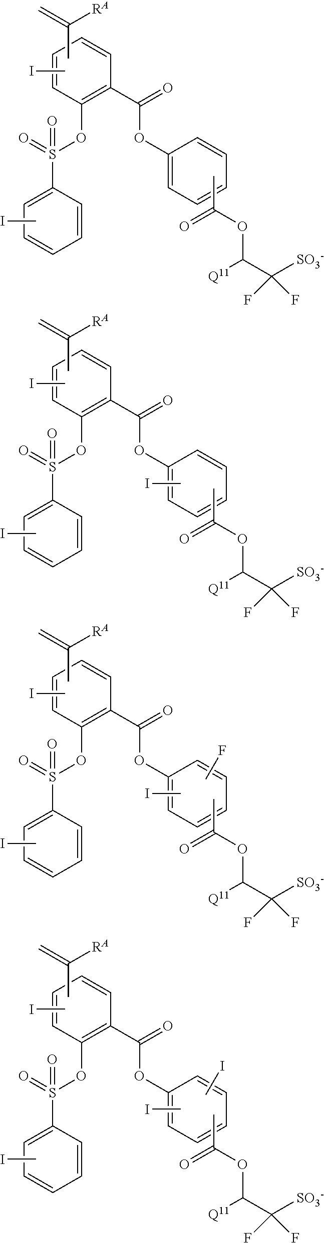

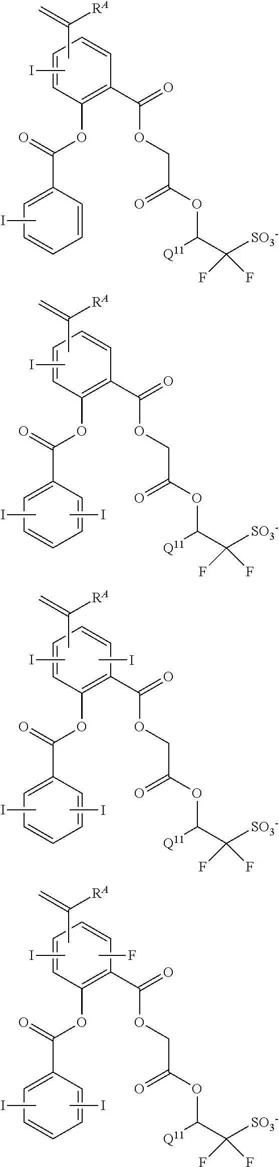

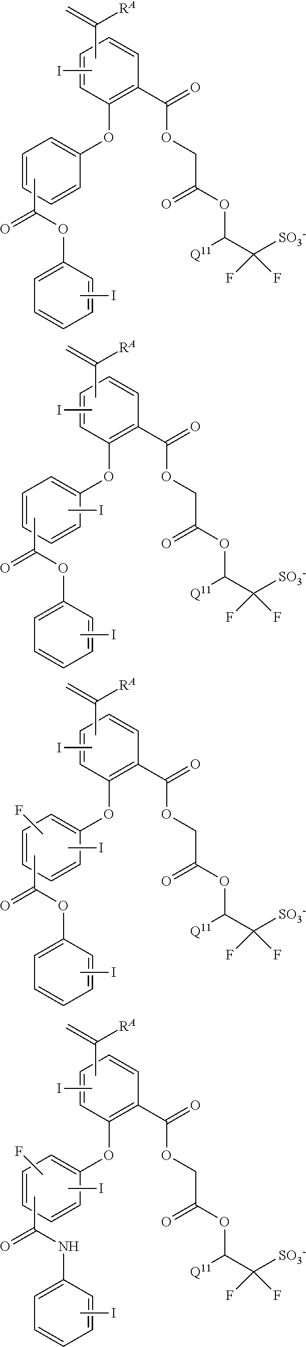

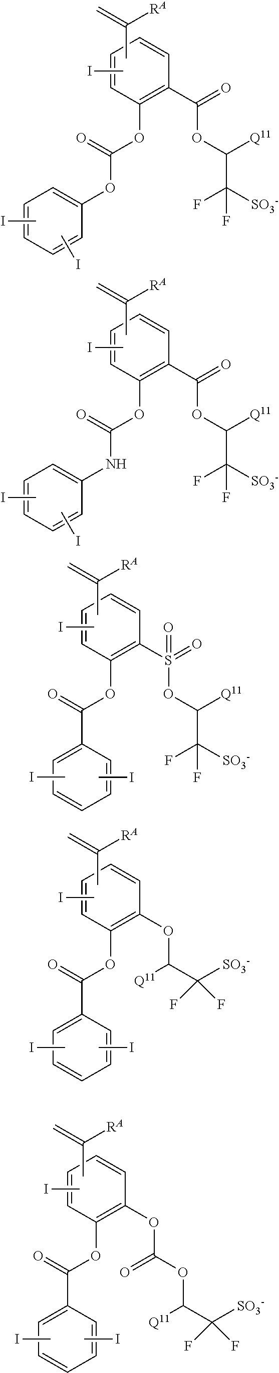

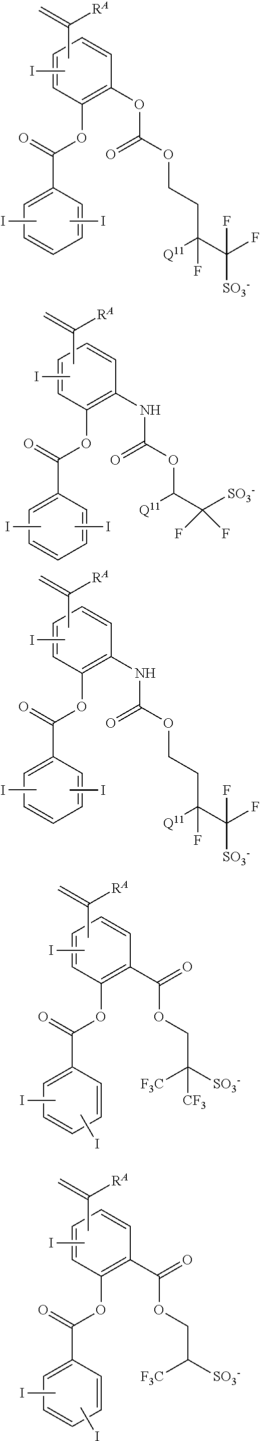

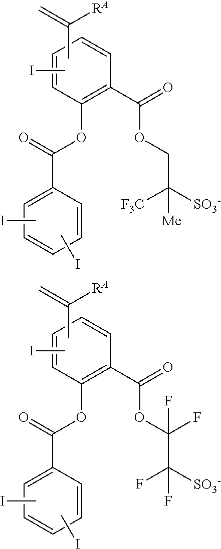

Specific examples of the sulfonium cation of the repeat unit d include, but are not limited to, those exemplified as a sulfonium cation of a sulfonium salt monomer having the formula (a), those described in paragraphs [0102] to [0125] of JP-A 2024-3744, those described in paragraphs [0044] to [0049] of WO 2024/128017, and those described in paragraphs [0035] to [0046] of JP 7491173.

The sulfonium cation of the repeat unit d is also preferably a sulfonium cation having the following formula (d3):

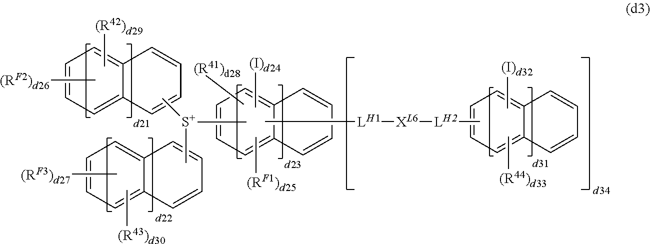

In the formula (d3), d21 is 0 or 1. When d21 is 0, it is a benzene ring, and when d21 is 1, it is a naphthalene ring, but a benzene ring in which d21 is 0 is preferable from a viewpoint of solvent solubility. d22 is 0 or 1. When d22 is 0, it is a benzene ring, and when d22 is 1, it is a naphthalene ring, but a benzene ring in which d22 is 0 is preferable from a viewpoint of solvent solubility. d23 is 0 or 1. When d23 is 0, it is a benzene ring, and when d23 is 1, it is a naphthalene ring, but a benzene ring in which d23 is 0 is preferable from a viewpoint of solvent solubility.

In the formula (d3), d24 is 0, 1, 2, 3, or 4. The larger the number of iodine atoms in the cation structure, the higher the absorption particularly for EUV, but since there is a concern that solvent solubility is poor and precipitation occurs in a resist composition, d24 is preferably 0, 1, 2, or 3, and more preferably 0, 1, or 2.

In the formula (d3), d25 is 0, 1, 2, 3, or 4. d25 is preferably 0, 1, 2, or 3, and more preferably 0, 1, or 2 from a viewpoint of raw material procurement. d26 is 0, 1, 2, 3, 4, 5, or 6. d26 is preferably 0, 1, 2, or 3, and more preferably 0, 1, or 2 from a viewpoint of raw material procurement. d27 is 0, 1, 2, 3, 4, 5, or 6. d27 is preferably 0, 1, 2, or 3, and more preferably 0, 1, or 2 from a viewpoint of raw material procurement.

In the formula (d3), d28 is 0, 1, or 2. d28 is preferably 0 or 1 from a viewpoint of raw material procurement. d29 is 0, 1, or 2. d29 is preferably 0 or 1 from a viewpoint of raw material procurement. d30 is 0, 1, or 2. d30 is preferably 0 or 1 from a viewpoint of raw material procurement.

In the formula (d3), d31 is 0 or 1. When d31 is 0, it is a benzene ring, and when d31 is 1, it is a naphthalene ring, but a benzene ring in which d31 is 0 is preferable from a viewpoint of solvent solubility.

In the formula (d3), d32 is 0, 1, 2, 3, or 4. The larger the number of iodine atoms in the cation structure, the higher the absorption particularly for EUV, but since there is a concern that solvent solubility is poor and precipitation occurs in a resist composition, d32 is preferably 0, 1, 2, or 3, and more preferably 0, 1, or 2.

In the formula (d3), d33 is 0, 1, or 2. d33 is preferably 0 or 1 from a viewpoint of raw material procurement. d34 is 0, 1, or 2. d34 is preferably 0 or 1 from a viewpoint of synthesis.

Provided that 0≤d26+d29≤4 when d21 is 0, and 0≤d26+d29≤6 when d21 is 1. Provided that 0≤d27+d30≤4 when d22 is 0, and 0≤d27+d30≤6 when d22 is 1. Provided that 1≤d24+d25+d28+d34≤4 when d23 is 0, and 1≤d24+d25+d28+d34≤6 when d23 is 1. Provided that 0≤d32+d33≤4 when d31 is 0, and 0≤d32+d33≤6 when d31 is 1. d24+d32≥1.

In the formula (d3), R^{F1} to R^{F3} are each independently a fluorine atom, a C₁-C₆ fluorinated saturated hydrocarbyl group, a C₁-C₆ fluorinated saturated hydrocarbyloxy group, or a C₁-C₆ fluorinated saturated hydrocarbylthio group. Among these, R^{F1} to R^{F3} are each preferably a trifluoromethyl group, a trifluoromethoxy group, or a trifluorothiomethoxy group. When d25 is 2 or more, R^{F1}s may be the same as or different from each other. When d26 is 2 or more, R^{F2}s may be the same as or different from each other. When d27 is 2 or more, R^{F3}s may be the same as or different from each other.

In the formula (d3), each of R⁴¹ to R⁴⁴ is a halogen atom other than an iodine atom and a fluorine atom, a nitro group, a cyano group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. The hydrocarbyl group and hydrocarbyl moieties of the hydrocarbyloxy group and the hydrocarbylthio group may be saturated or unsaturated, and may be linear, branched, or cyclic. Specific examples thereof include those similar to those exemplified as the hydrocarbyl group of R¹ to R³ in the description of the formula (a). Some or all of hydrogen atoms of the hydrocarbyl group and hydrocarbyl moieties of the hydrocarbyloxy group and the hydrocarbylthio group may be replaced with a group containing a heteroatom such as an oxygen atom, a sulfur atom, a nitrogen atom, or a halogen atom, some of (—CH₂—)s in the hydrocarbyl group may be replaced with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like.

When d28 is 2, two R⁴¹s may be the same as or different from each other, and the two R⁴¹s may be bonded to each other to form a ring together with carbon atoms to which they are bonded. When d29 is 2, two R⁴²s may be the same as or different from each other, and two R⁴²s may be bonded to each other to form a ring together with carbon atoms to which they are bonded. When d30 is 2, two R⁴³s may be the same as or different from each other, and two R⁴³s may be bonded to each other to form a ring together with carbon atoms to which they are bonded. When d33 is 2, two R⁴⁴s may be the same as or different from each other, and two R⁴⁴s may be bonded to each other to form a ring together with carbon atoms to which they are bonded. Specific examples of the ring formed at this time include a cyclopropane ring, a cyclobutane ring, a cyclopentane ring, a cyclohexane ring, a norbornane ring, and an adamantane ring. Some or all of hydrogen atoms of the ring may be replaced with a group containing a heteroatom such as an oxygen atom, a sulfur atom, a nitrogen atom, or a halogen atom, some of (—CH₂—)s in the ring may be replaced with a group containing a heteroatom such as an oxygen atom, a sulfur atom, or a nitrogen atom, and as a result, the ring may contain a hydroxy group, a fluorine atom, a chlorine atom, a bromine atom, an iodine atom, a cyano group, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like.

Aromatic rings directly bonded to S⁺ in the sulfonium cation having the formula (d3) may be bonded to each other to form a ring together with S⁺. At this time, specific examples of the structure of the ring include those having the following formulas:

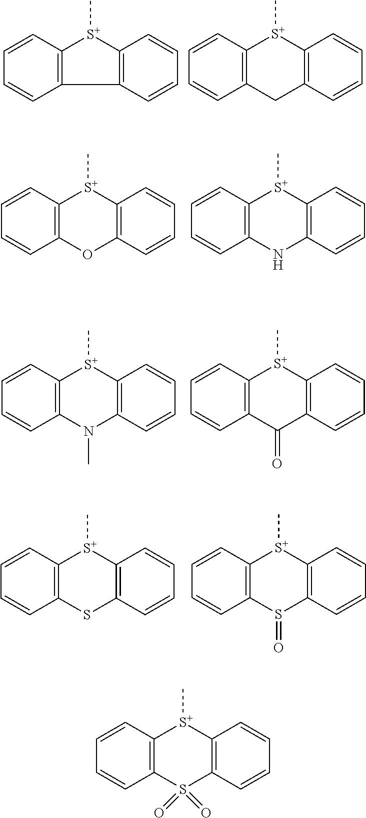

wherein the dashed line is a bond.

In the formula (d3), L^{H1} and L^{H2} are each independently a single bond, an ether bond, an ester bond, an amide bond, a sulfonate bond, a sulfonamide bond, a carbonate bond, or a carbamate bond. Among these, L^{H1} is preferably a single bond, an ether bond, an ester bond, or a sulfonate bond, and more preferably an ester bond or a sulfonate bond. L^{H2} is preferably a single bond, an ether bond, or an ester bond, and more preferably a single bond.

In the formula (d3), X^{L6} is a single bond or a C₁-C₄₀ hydrocarbylene group which may contain a heteroatom. The hydrocarbylene group may be linear, branched, or cyclic, and specific examples thereof include an alkanediyl group, a cyclic saturated hydrocarbylene group, and an arylene group. Specific examples of the heteroatom include an oxygen atom, a nitrogen atom, and a sulfur atom. Specific examples of the C₁-C₄₀ hydrocarbylene group of X^{L6} which may contain a heteroatom include X^{L}-0 to X^{L}-61 exemplified as specific examples of the C₁-C₄₀ hydrocarbylene group of X^{L1} and X^{L2} which may contain a heteroatom in the description of the formula (Z). Among these, X¹-0 to X^{L}-22, X^{L}-29 to X^{L}-34, and X^{L}-47 to X^{L}-61 are preferable.

The sulfonium cation having the formula (d3) preferably has the following formula (d3-1):

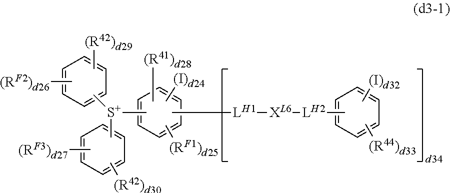

wherein d24 to d30, d32 to d34, R^{F1} to R^{F3}, R⁴¹ to R⁴⁴, L^{H1}, L^{H2}, and X^{L6} are as defined above.

The sulfonium cation having the formula (d3-1) preferably has the following formula (d3-2):

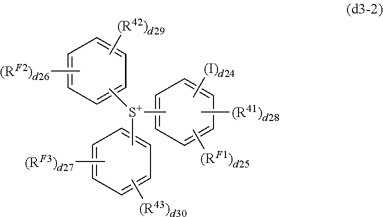

wherein d24 to d30, R^{F1} to R^{F3}, and R⁴¹ to R⁴³ are as defined above.

Specific examples of the sulfonium cation having the formula (d3) include, but are not limited to, those listed below. Note that, in the following formulas, Me is a methyl group.

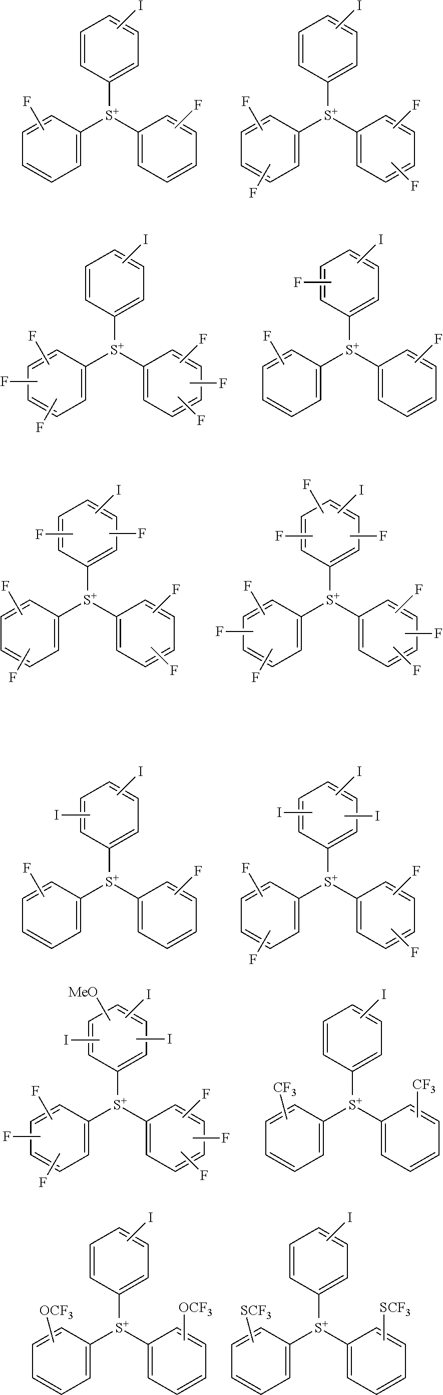

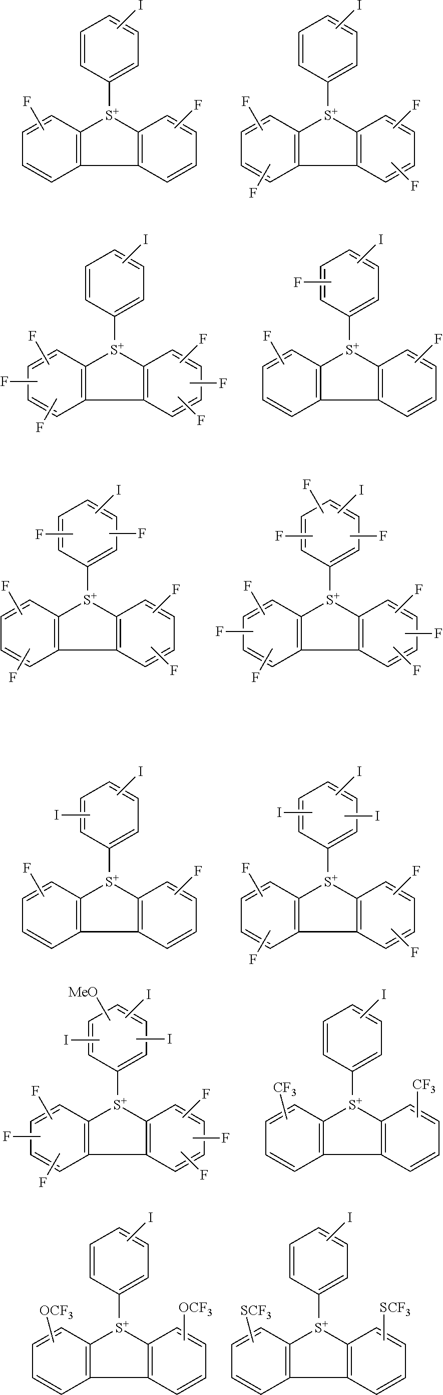

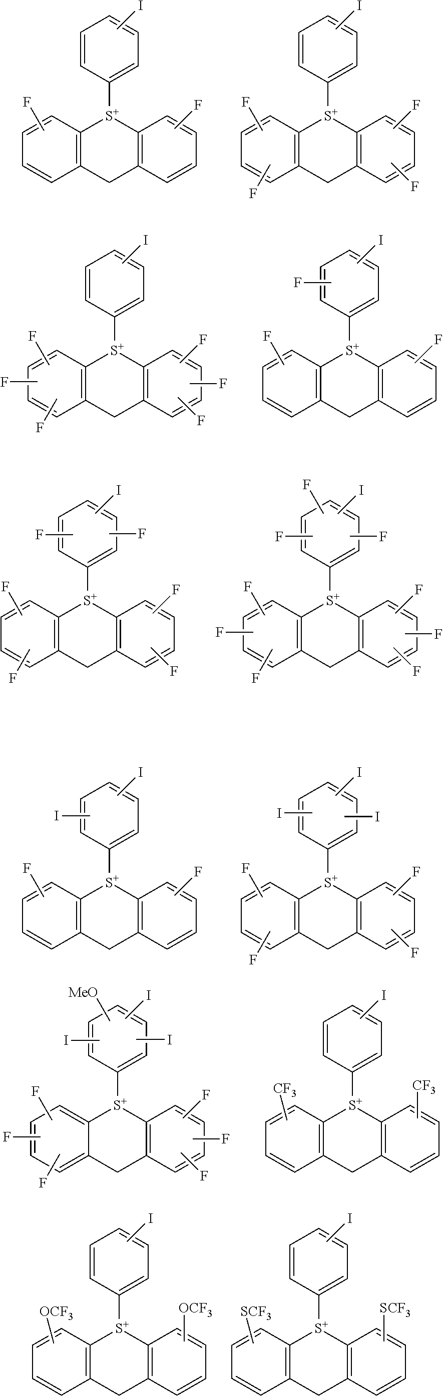

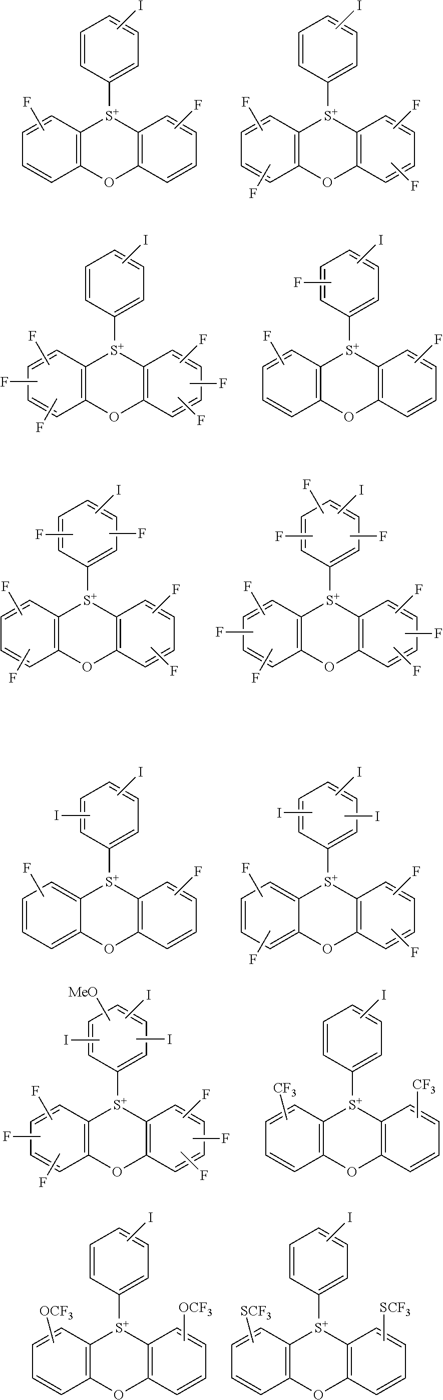

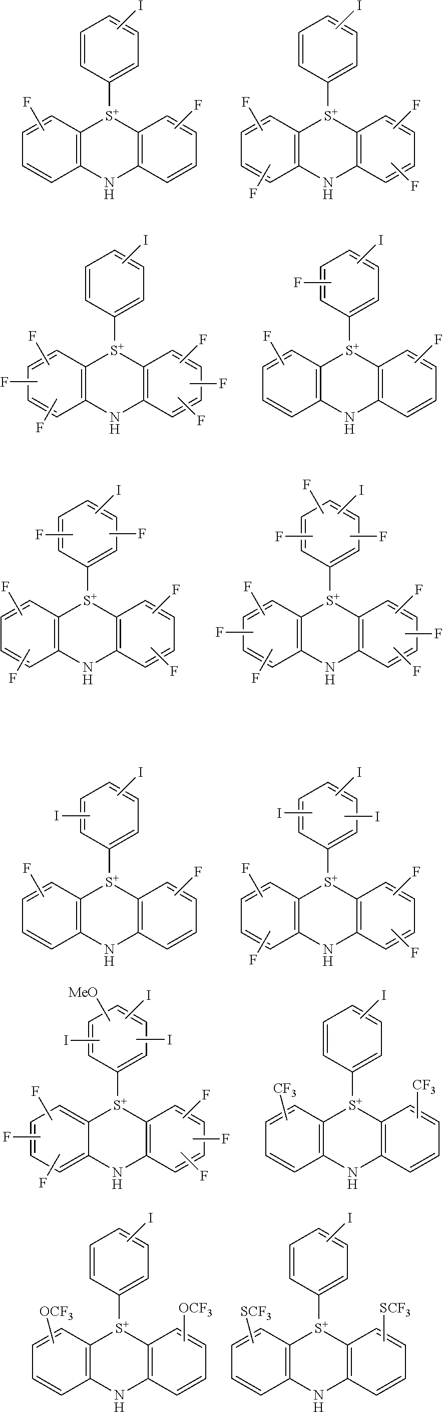

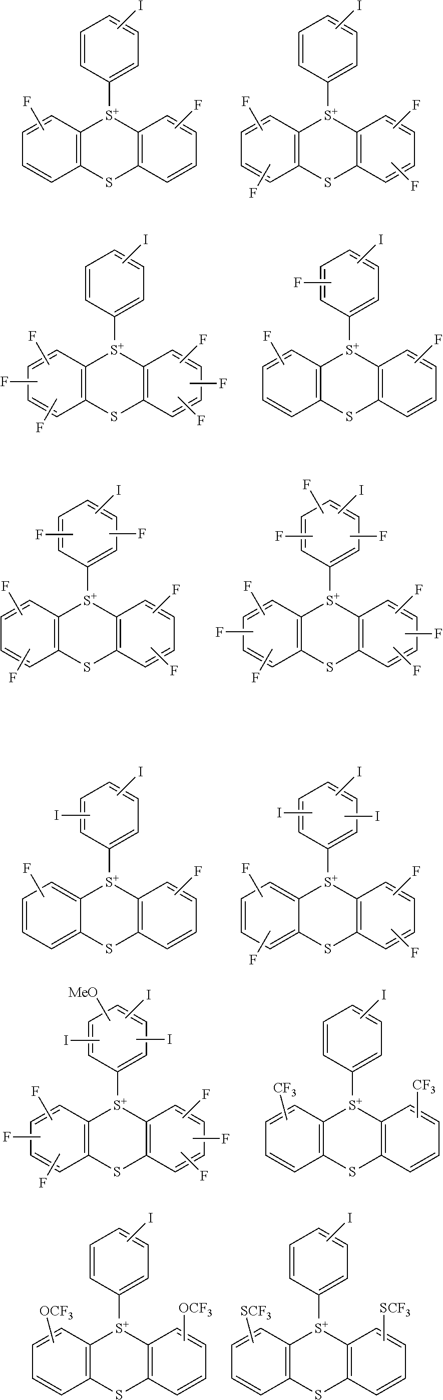

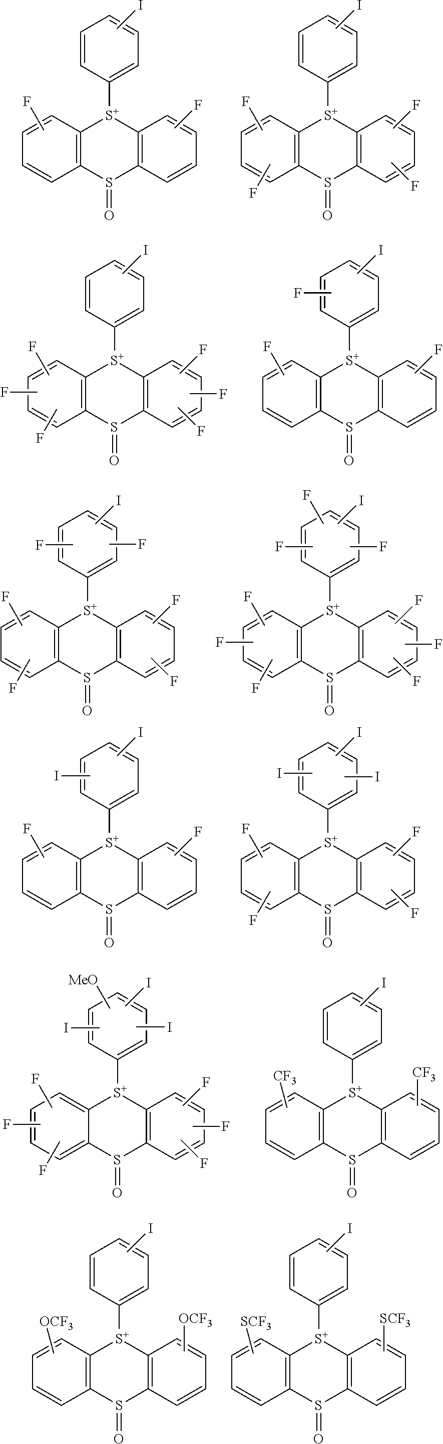

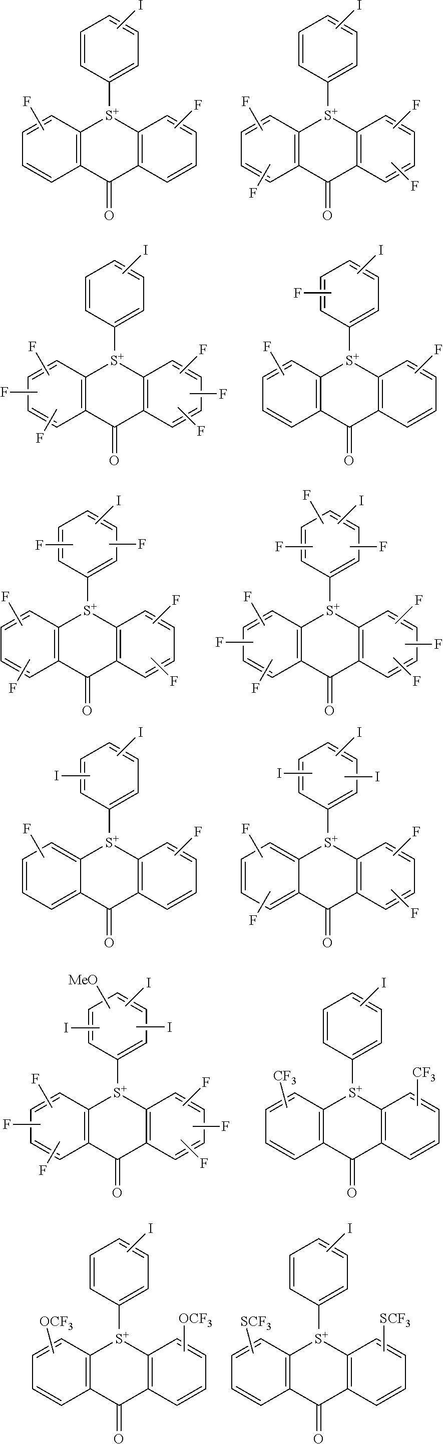

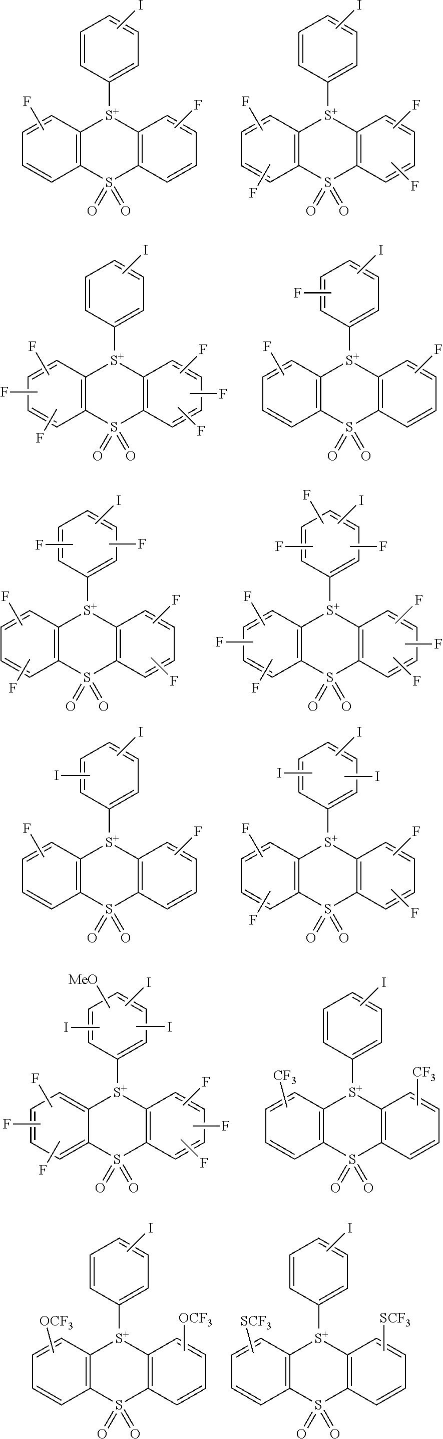

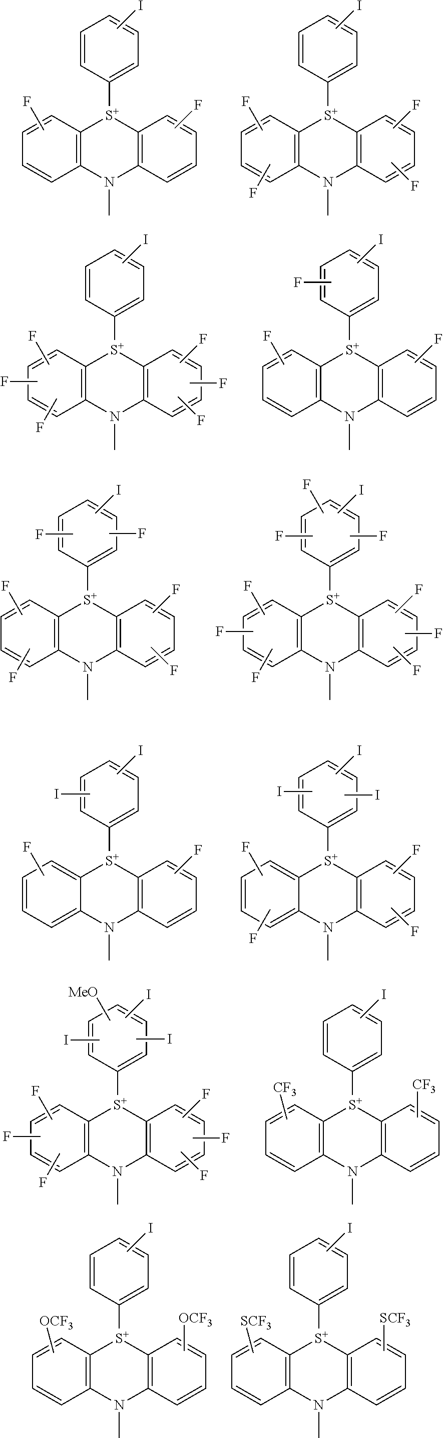

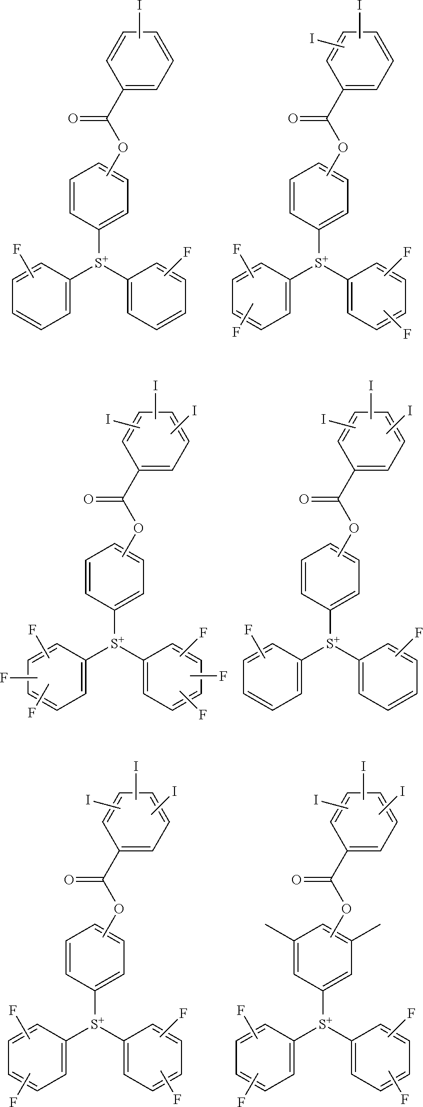

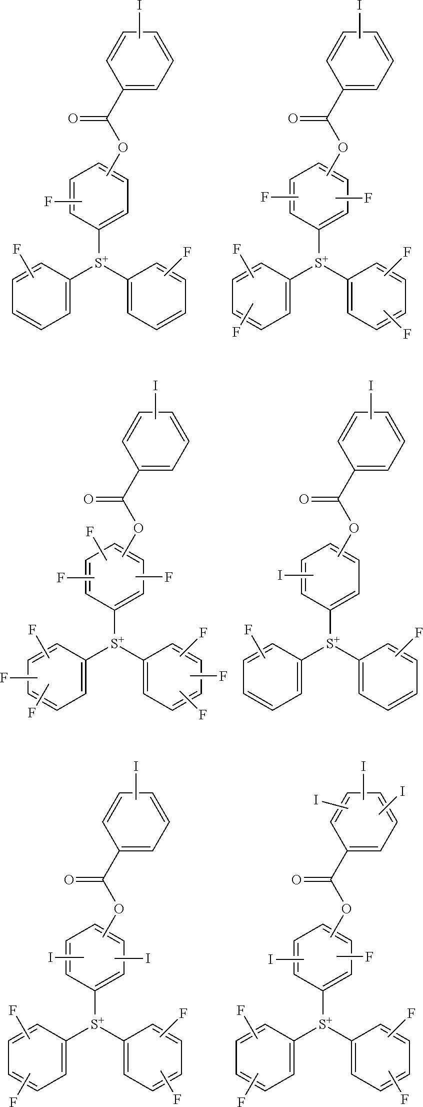

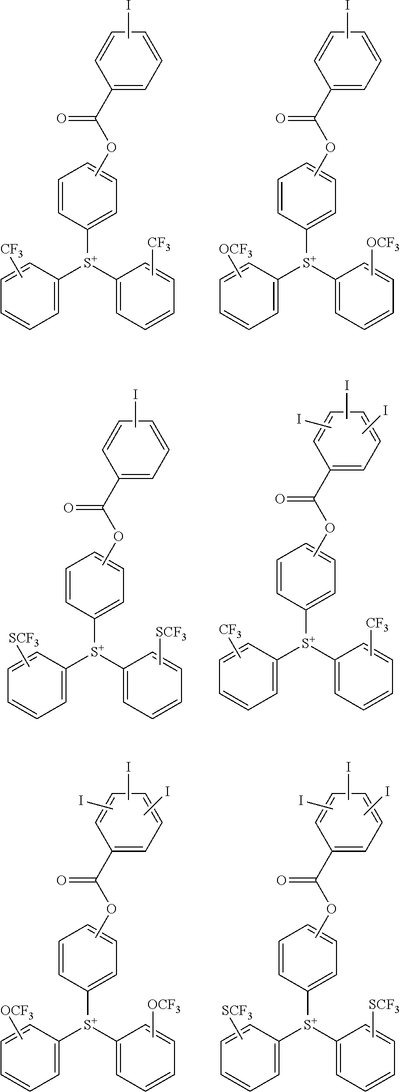

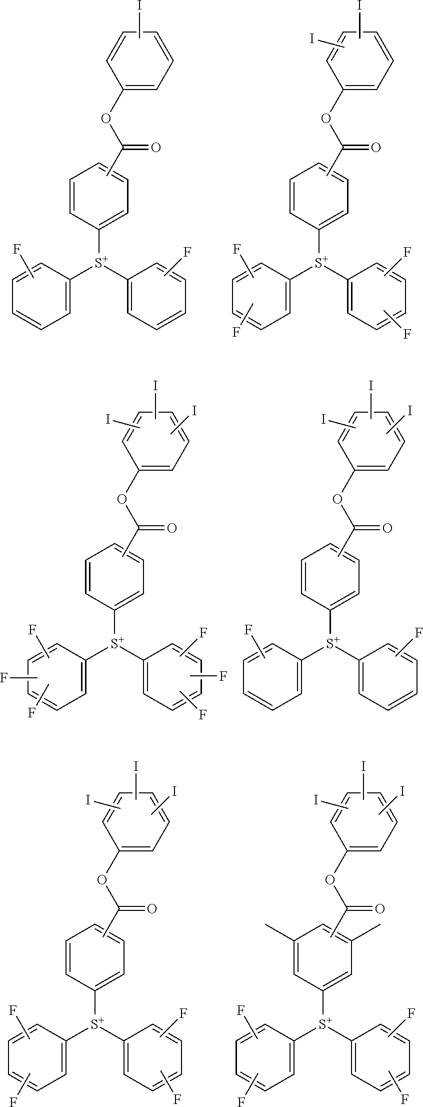

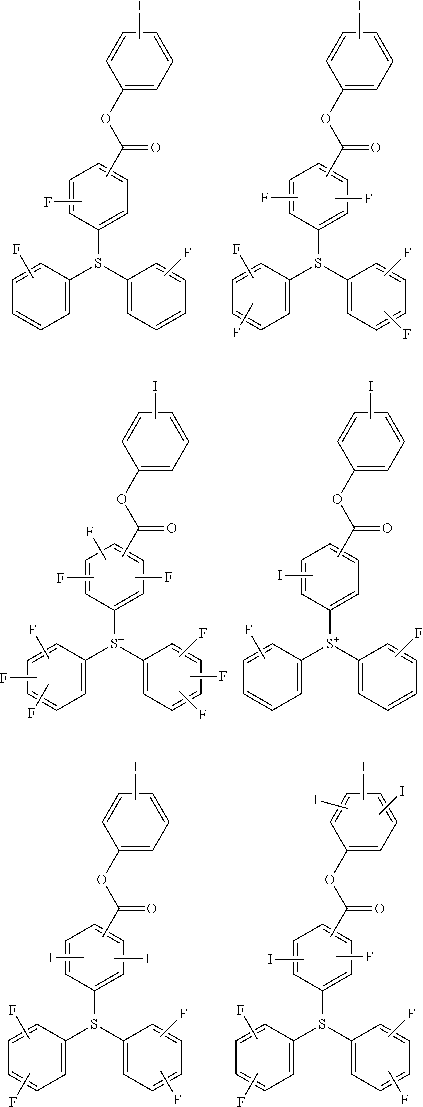

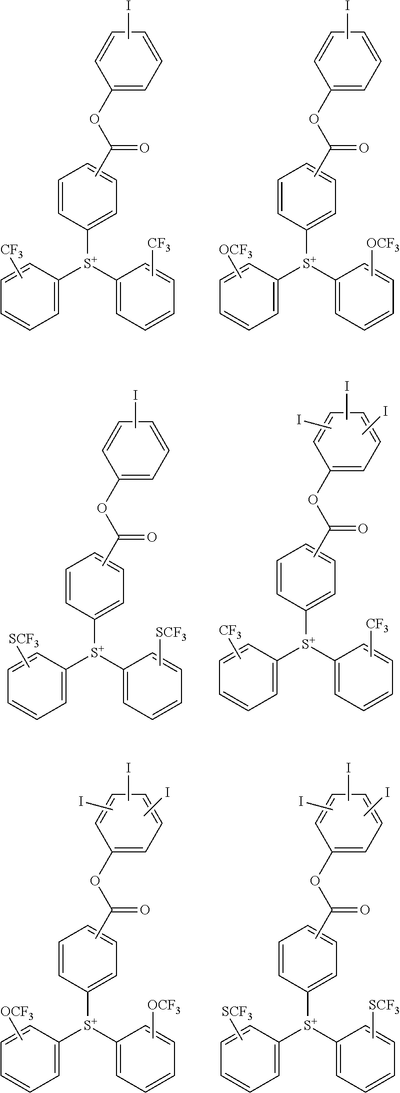

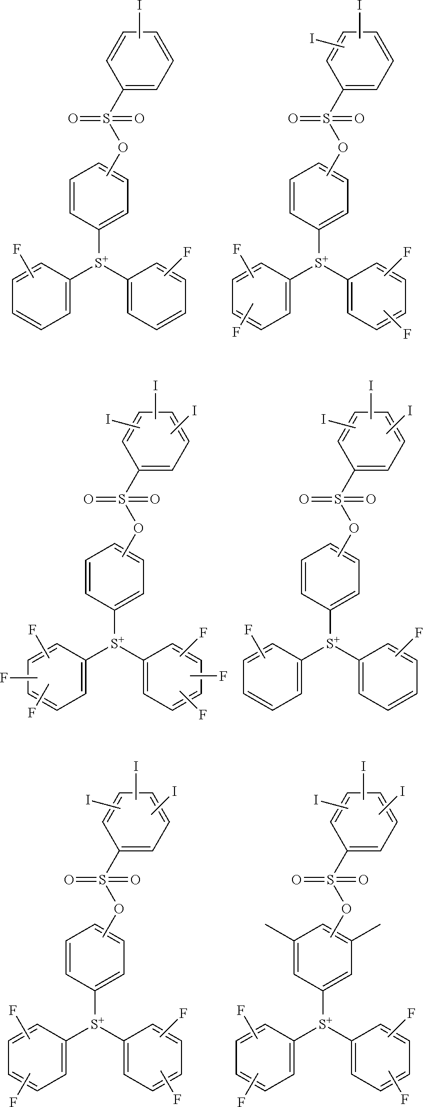

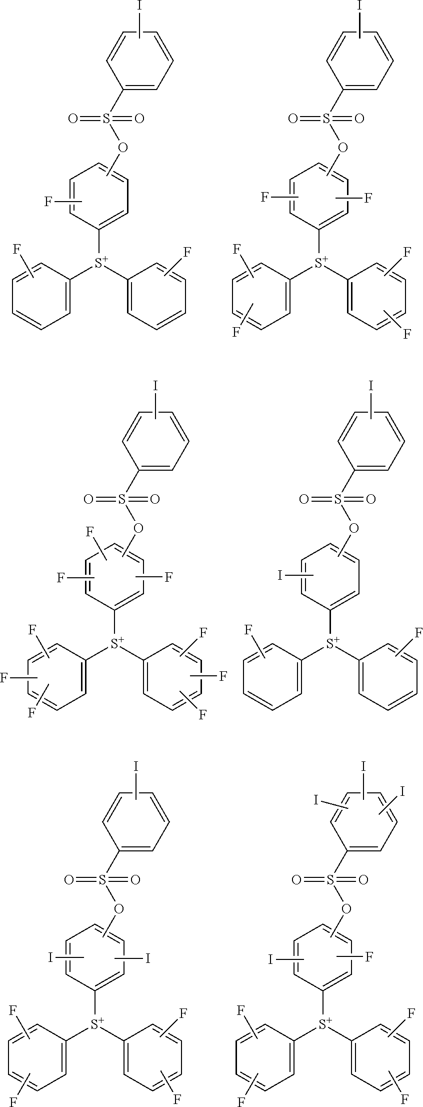

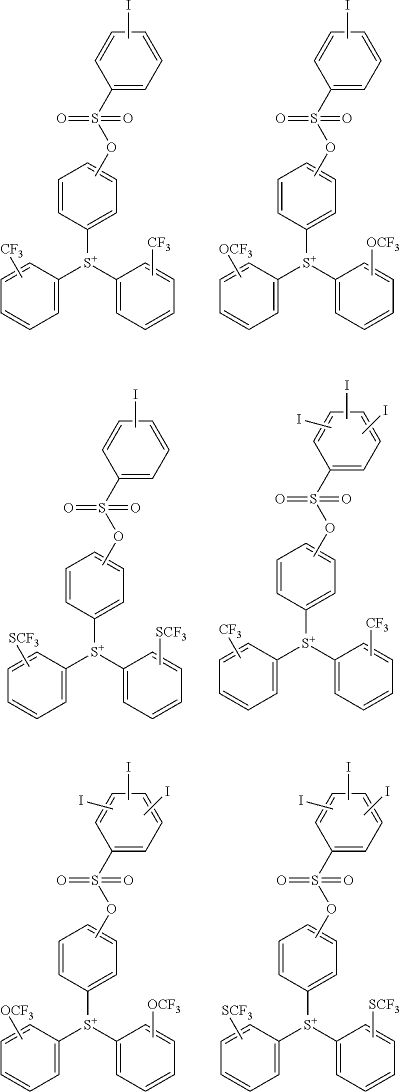

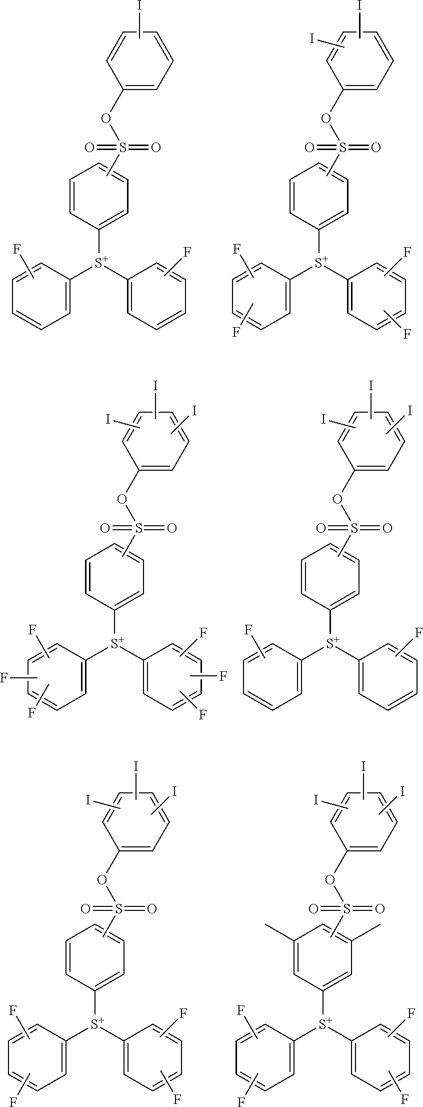

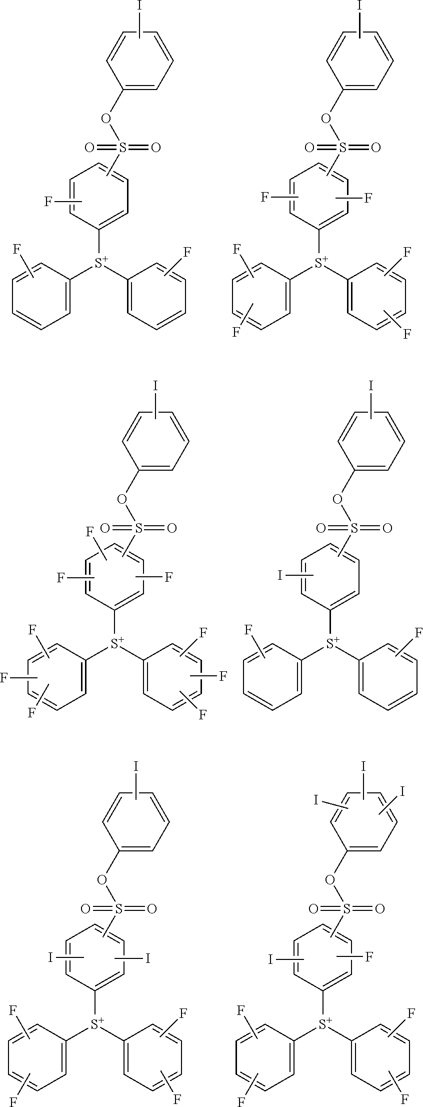

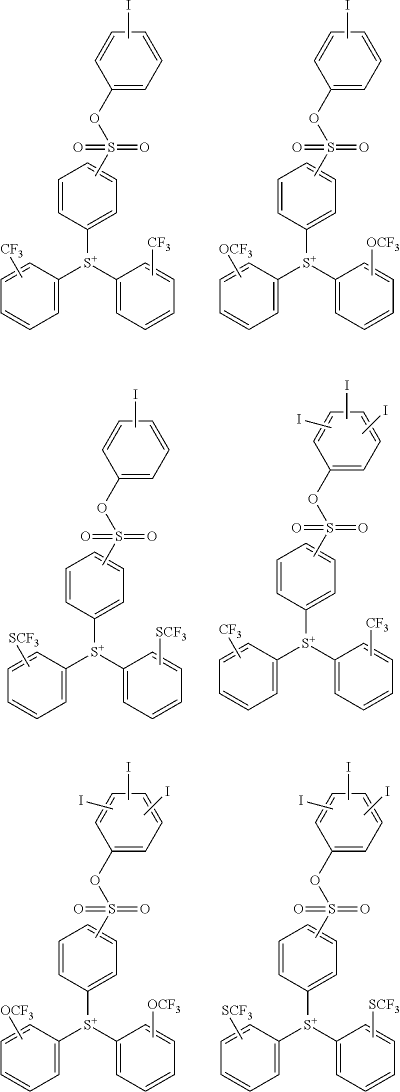

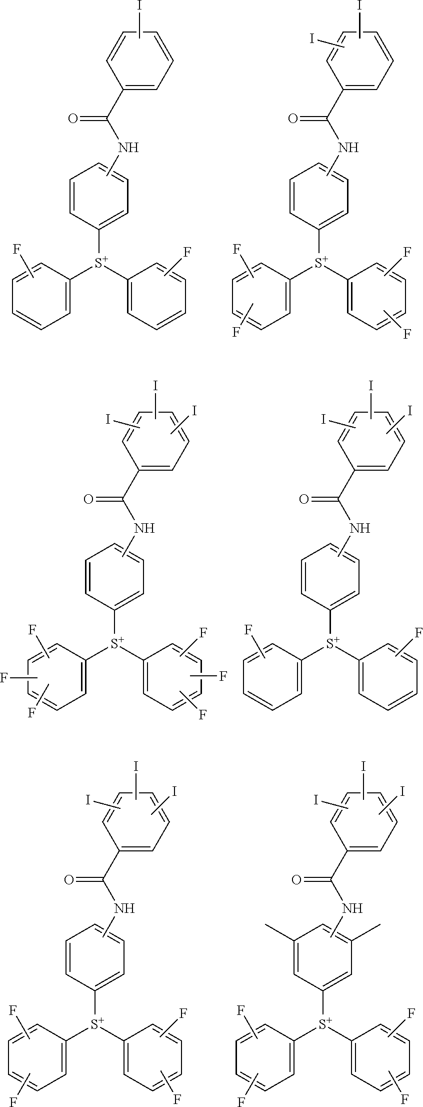

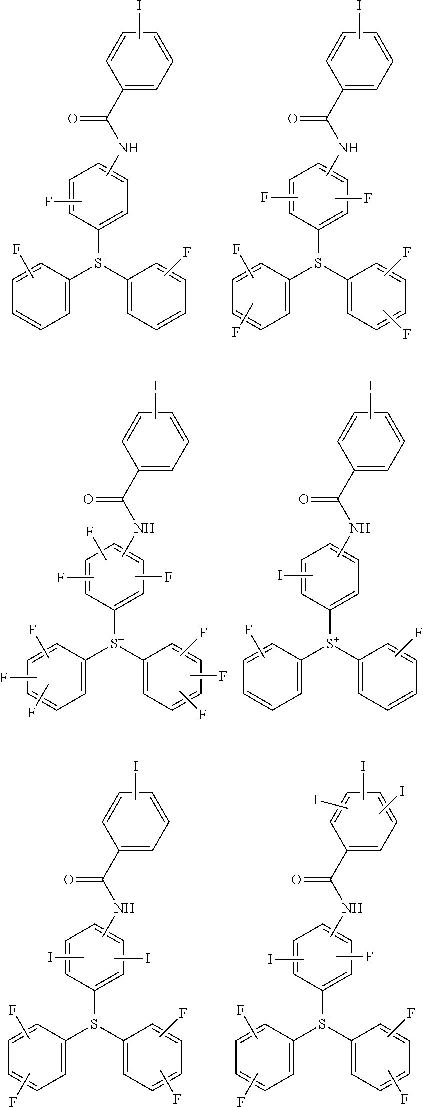

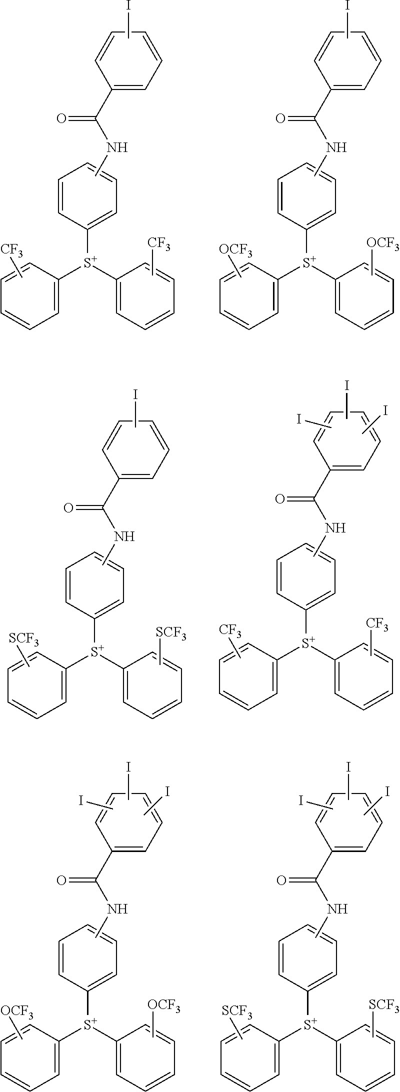

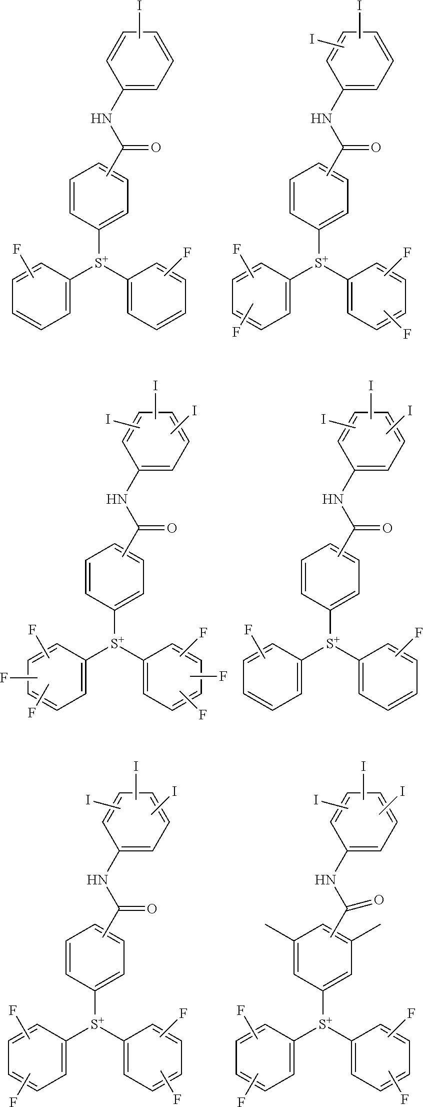

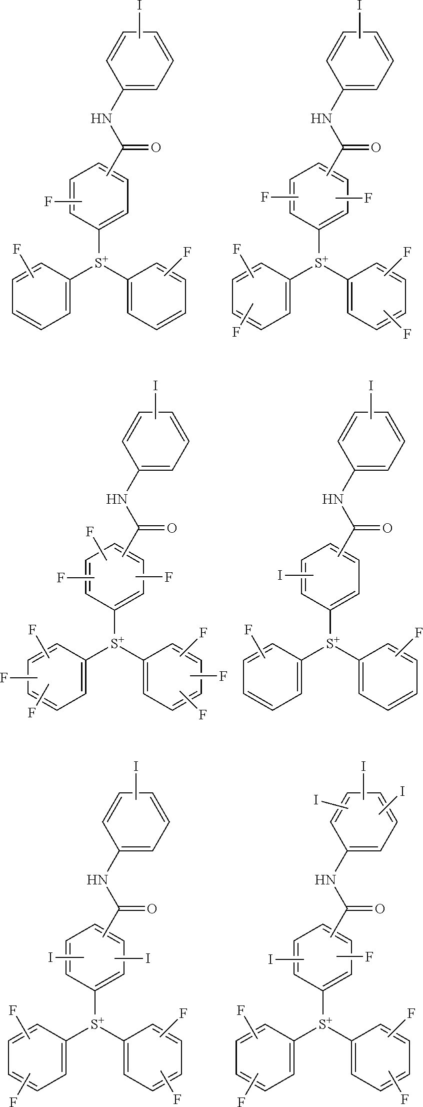

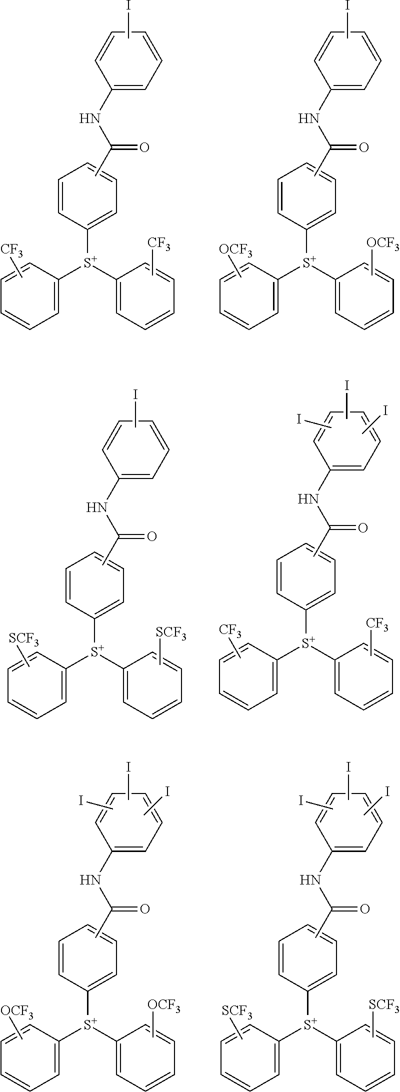

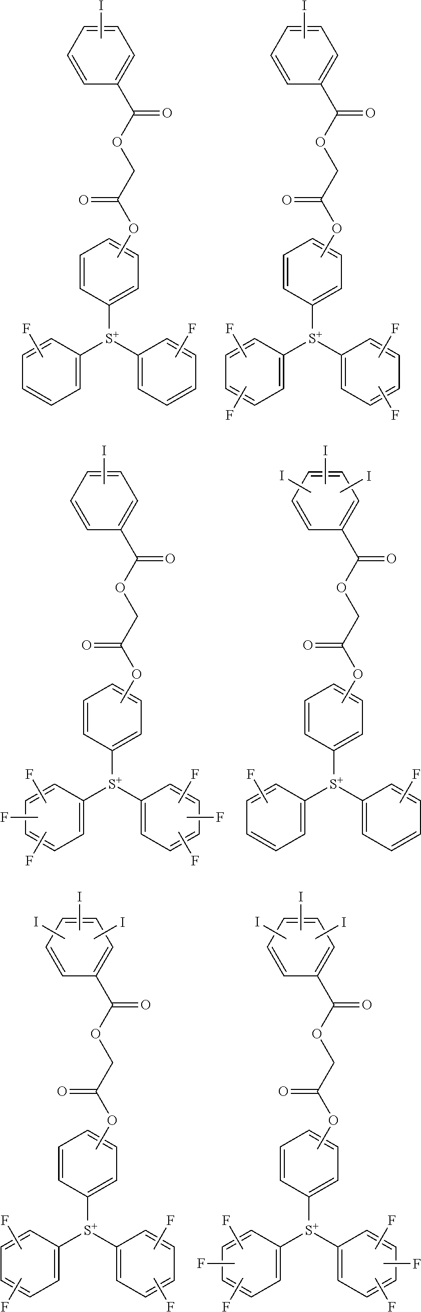

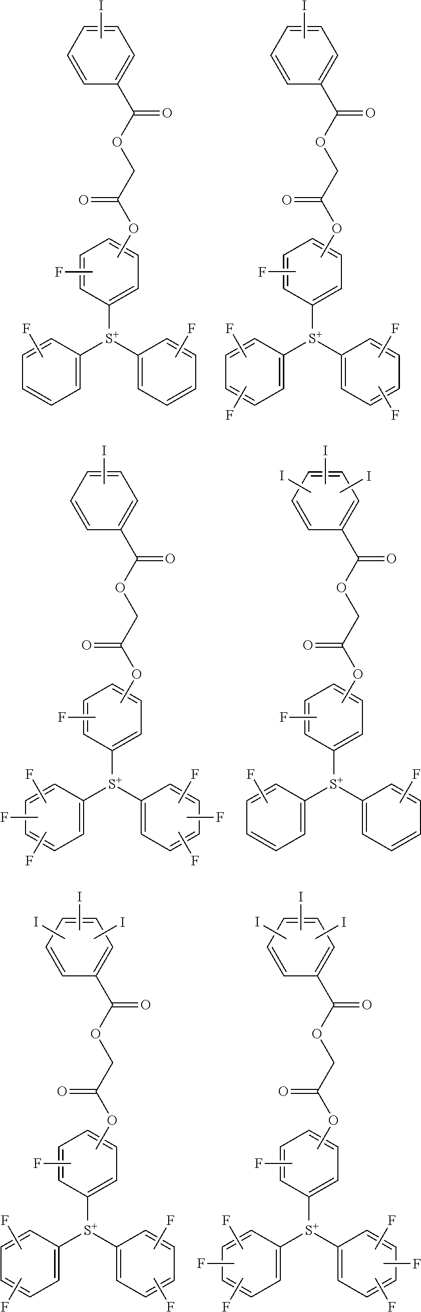

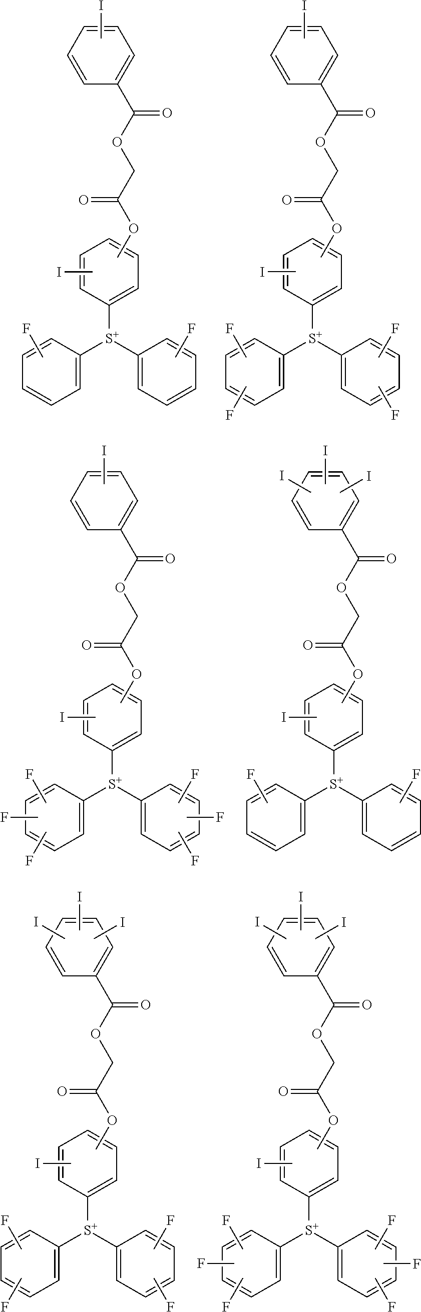

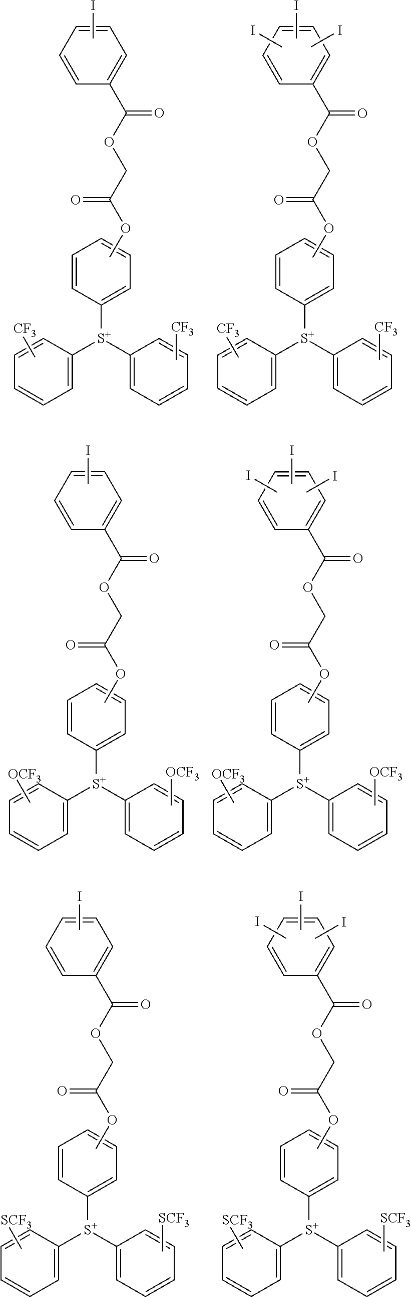

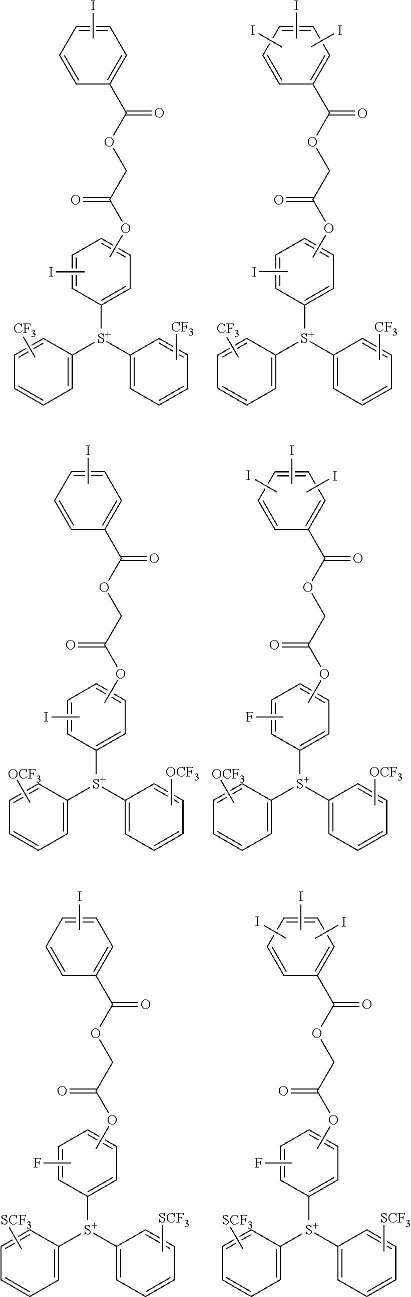

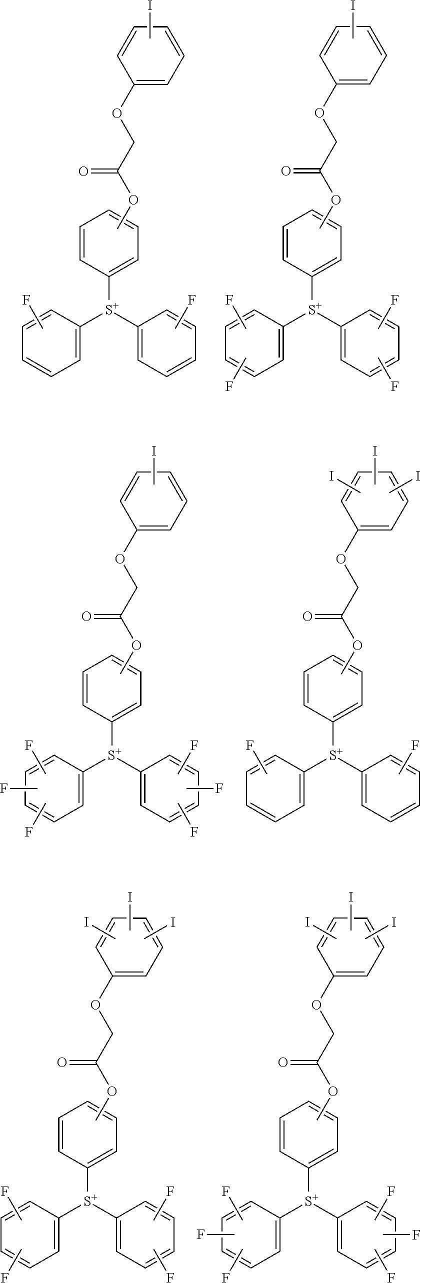

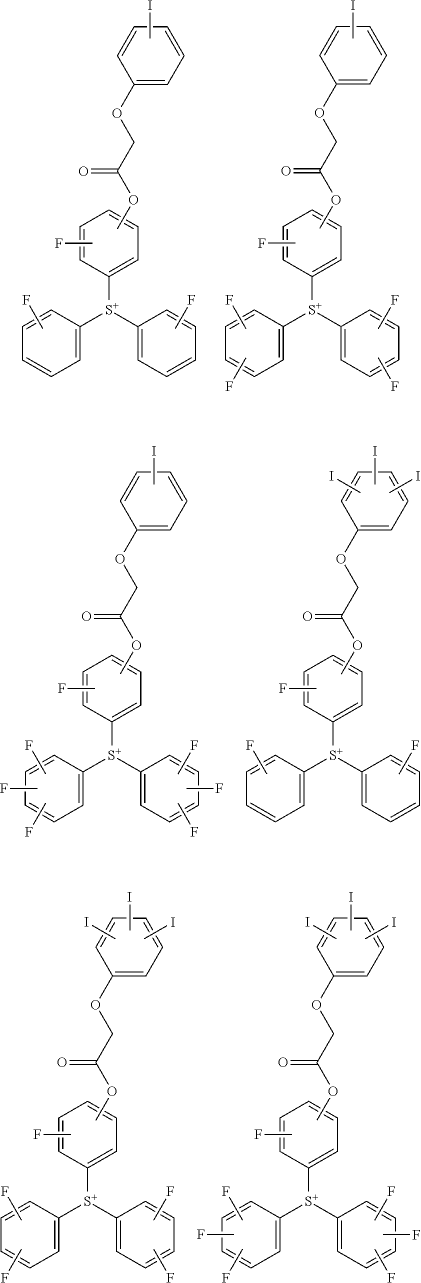

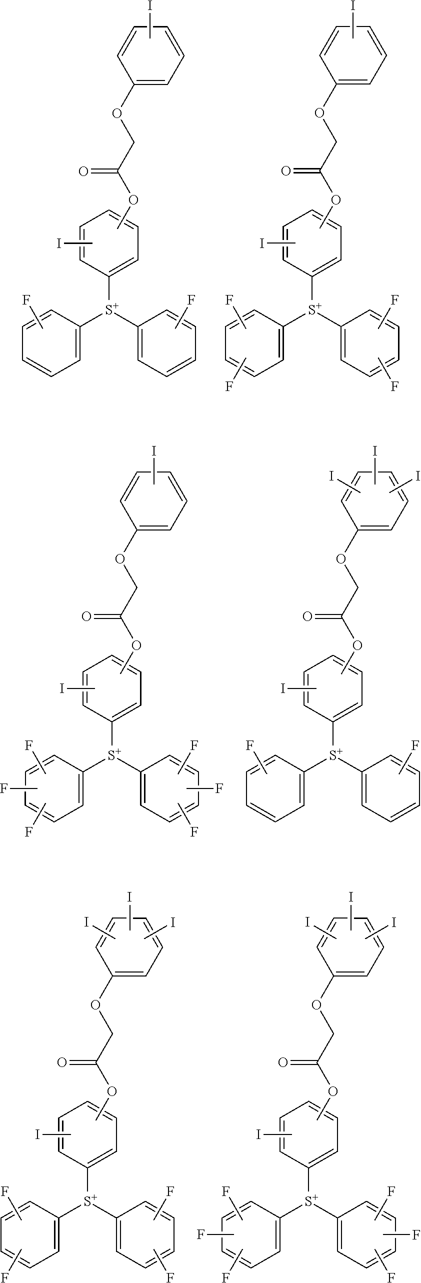

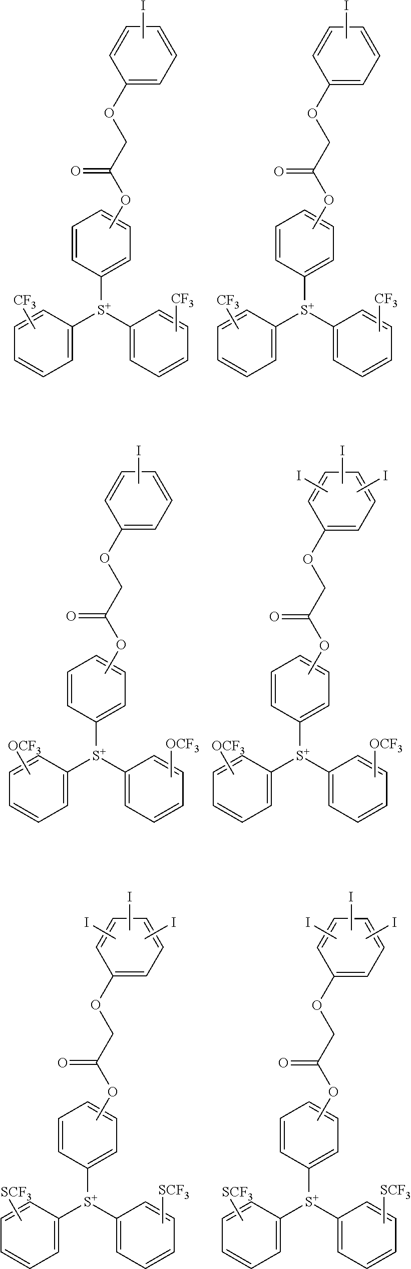

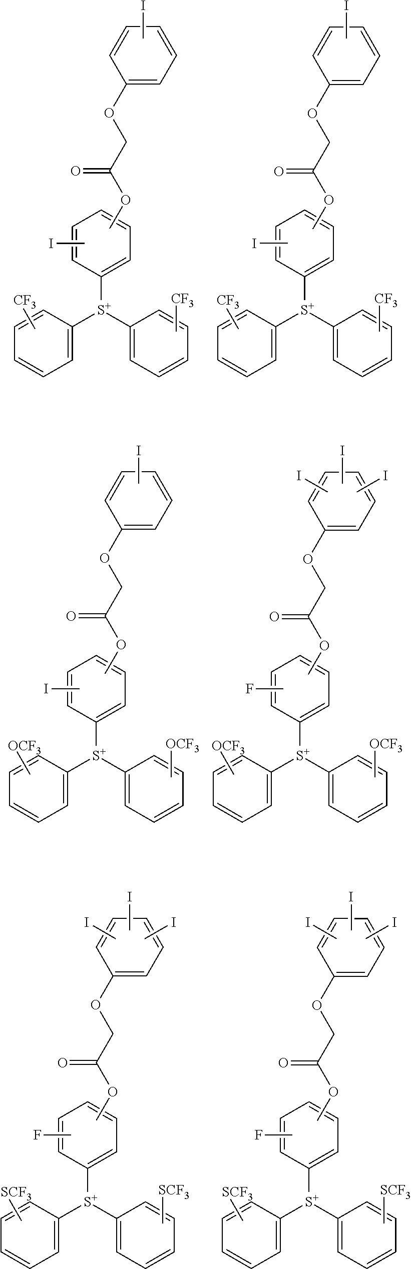

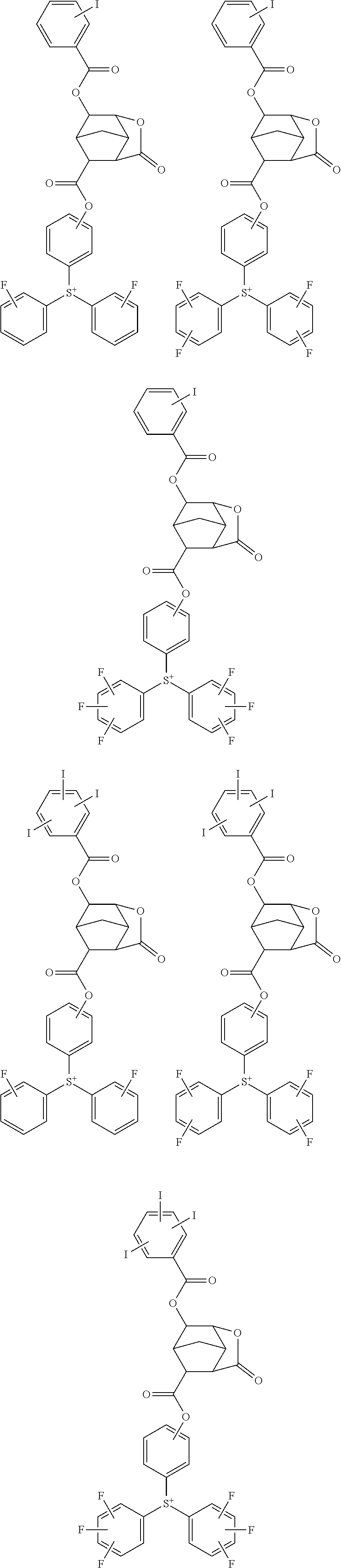

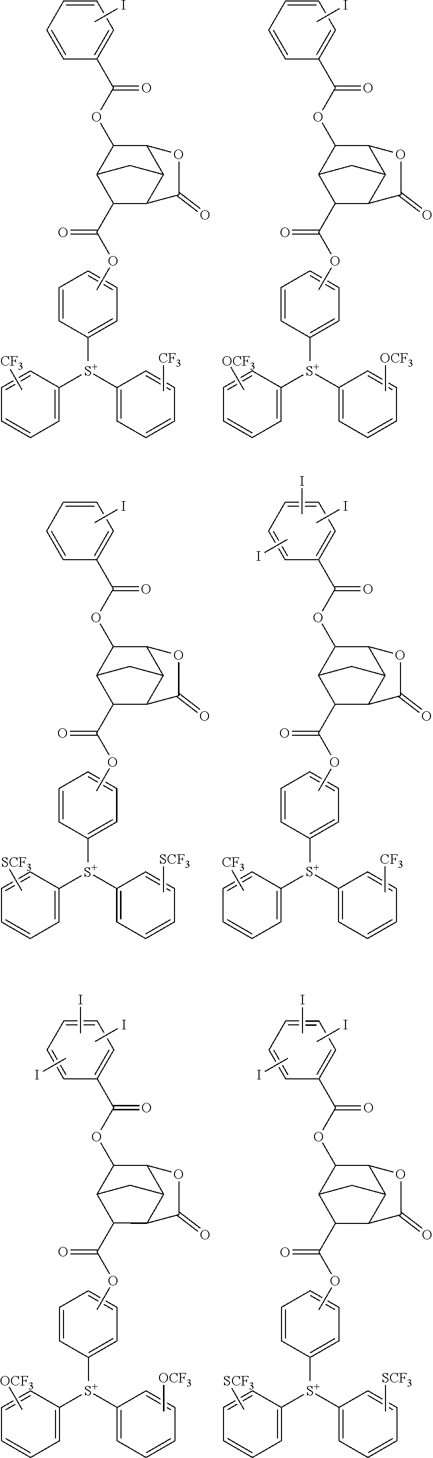

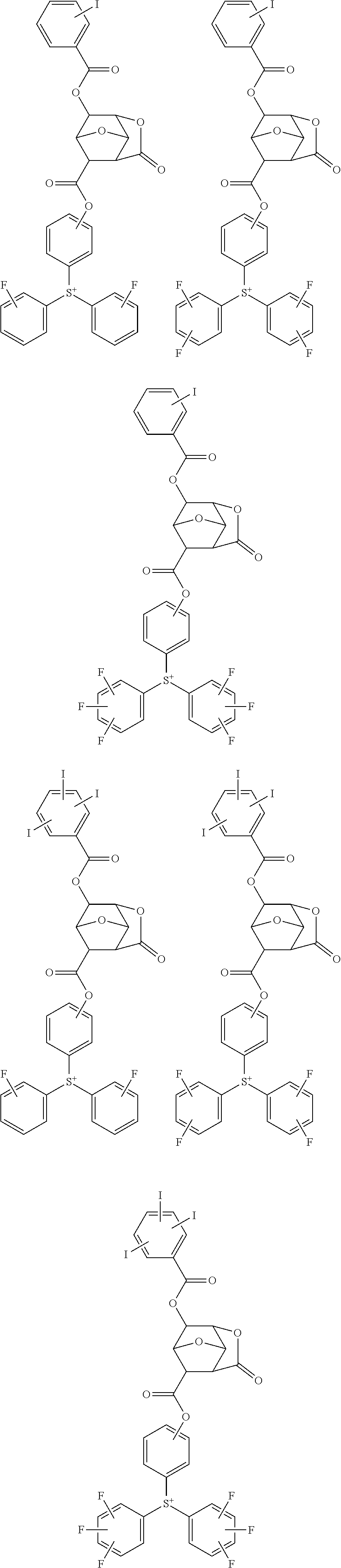

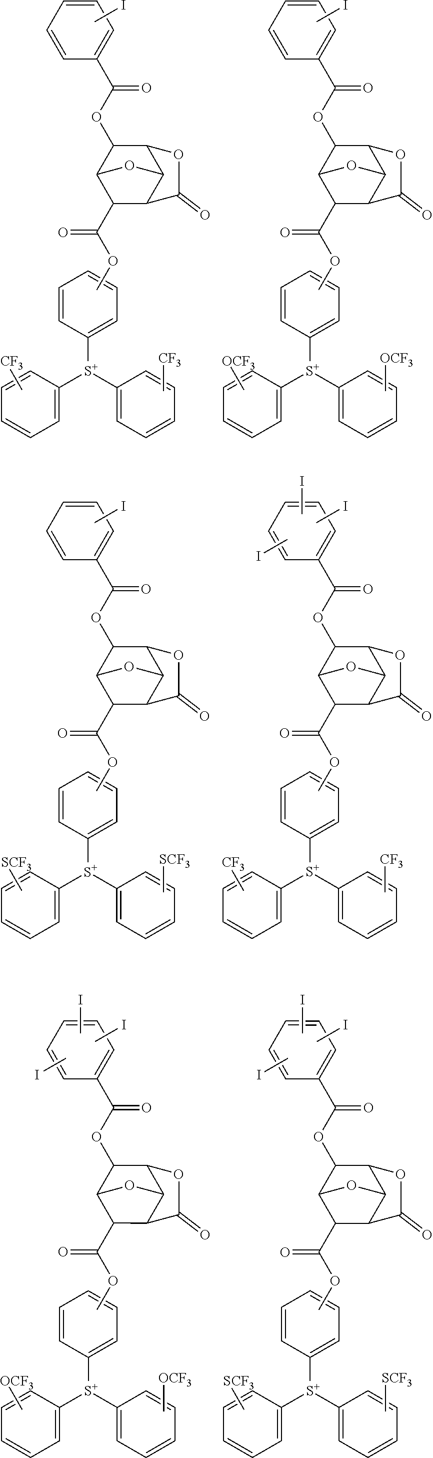

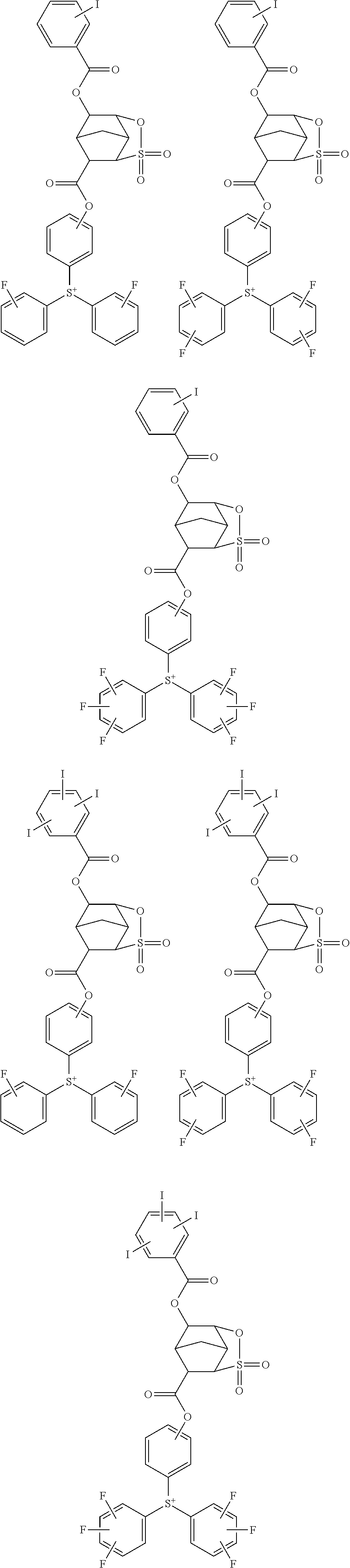

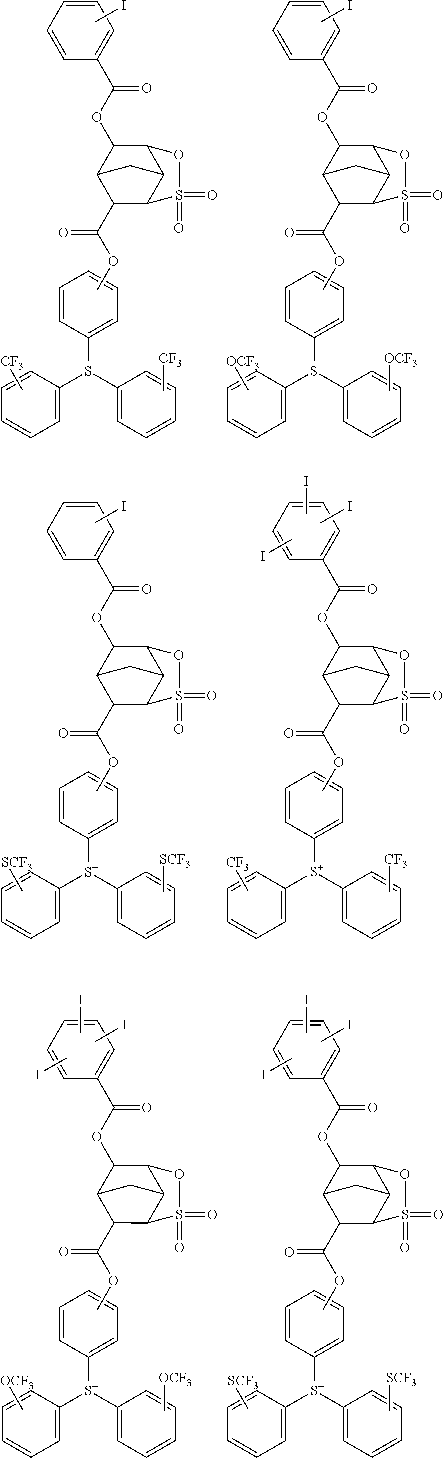

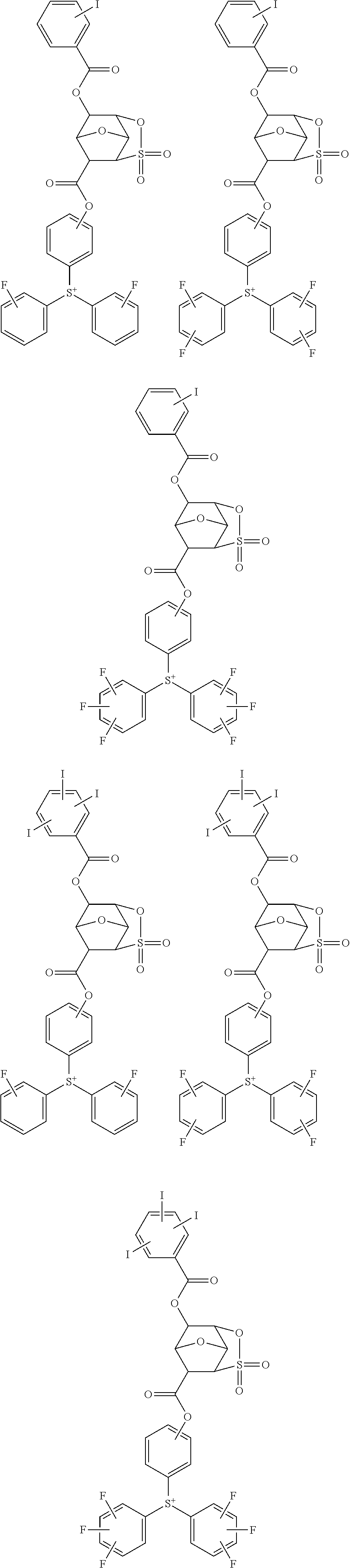

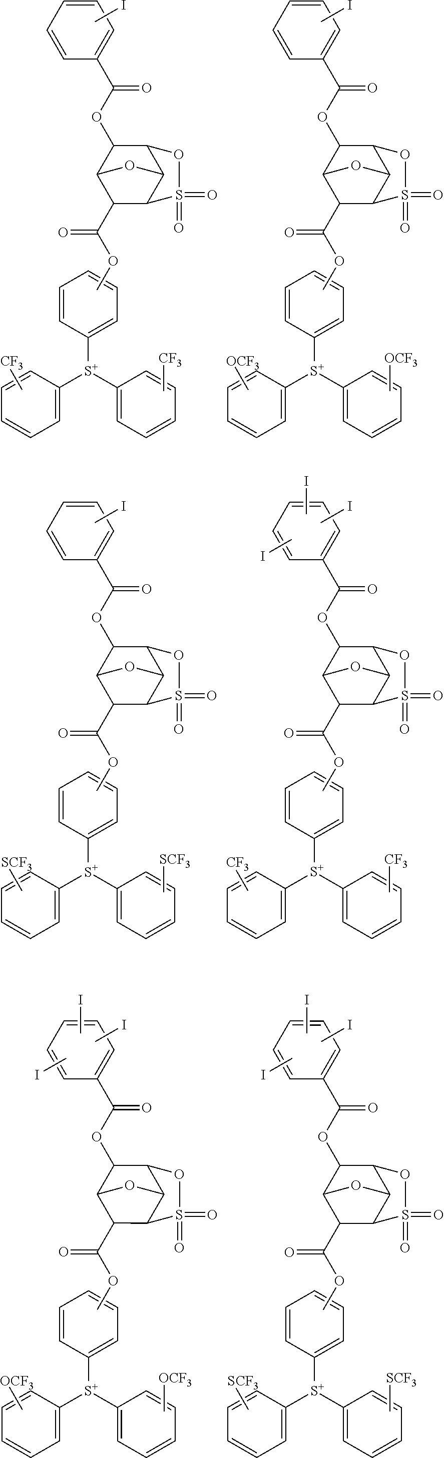

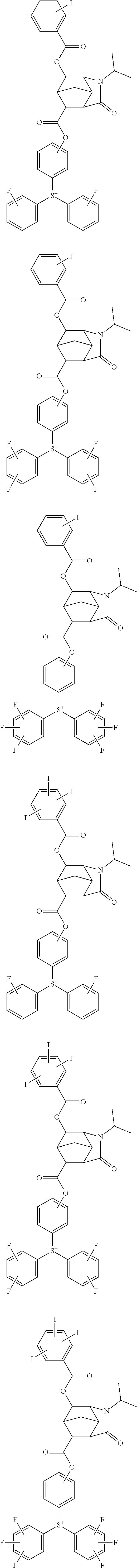

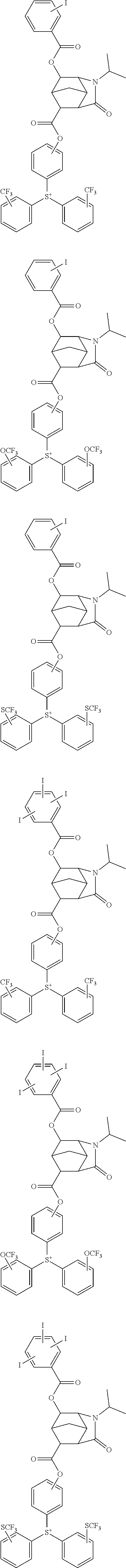

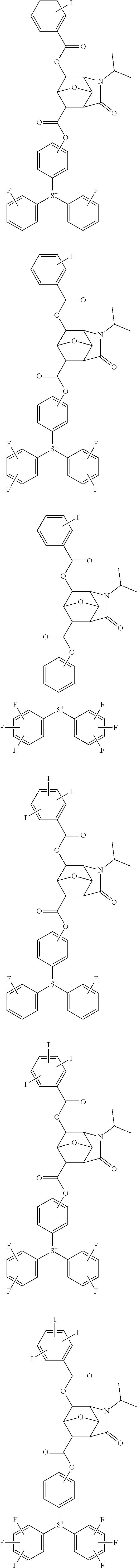

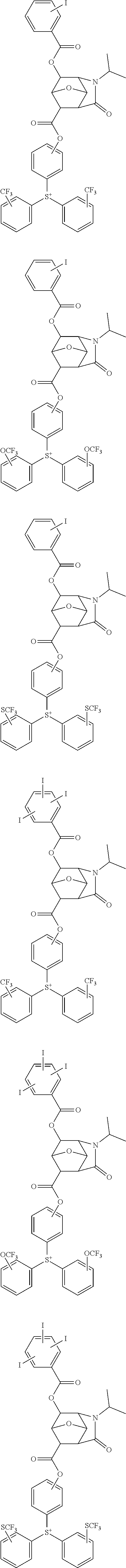

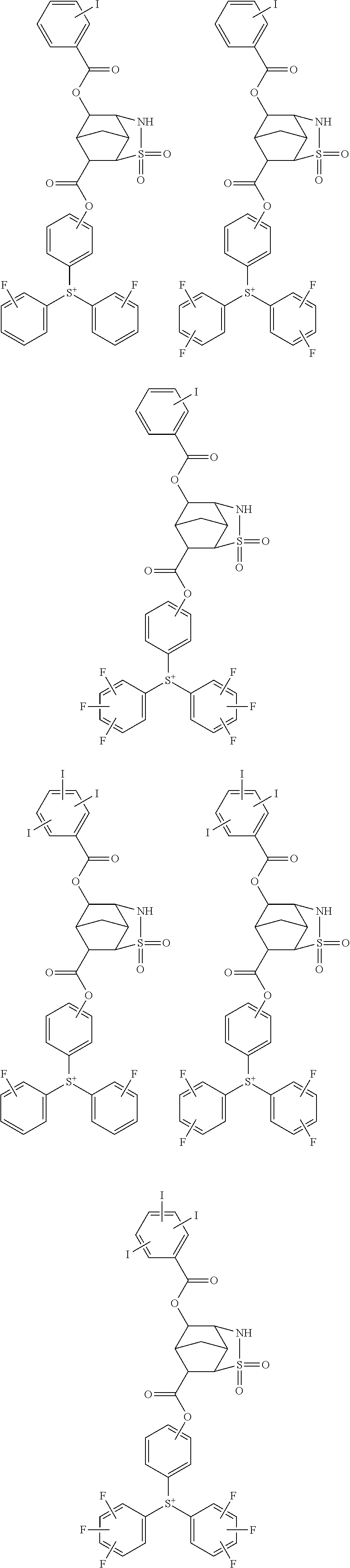

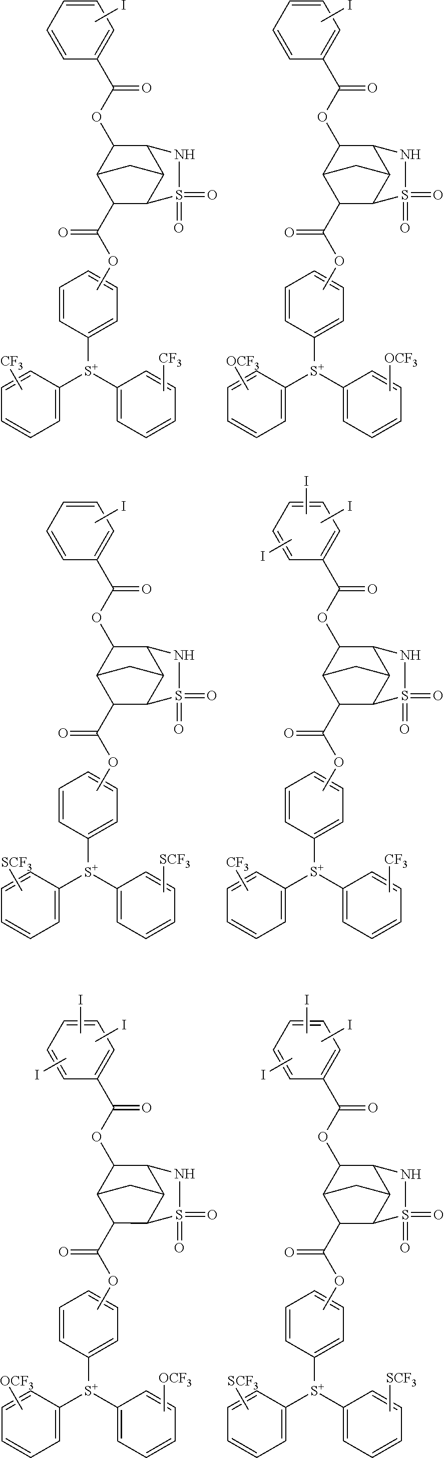

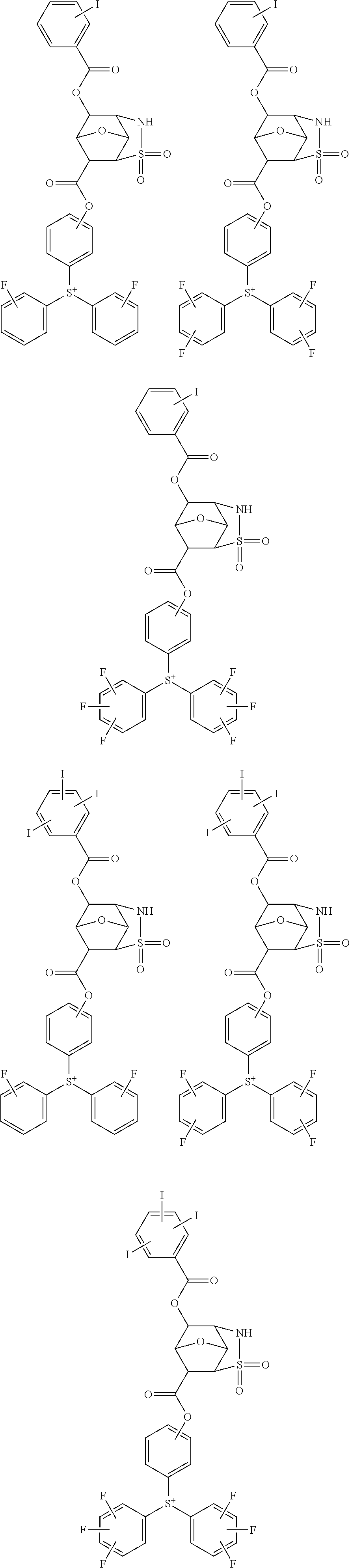

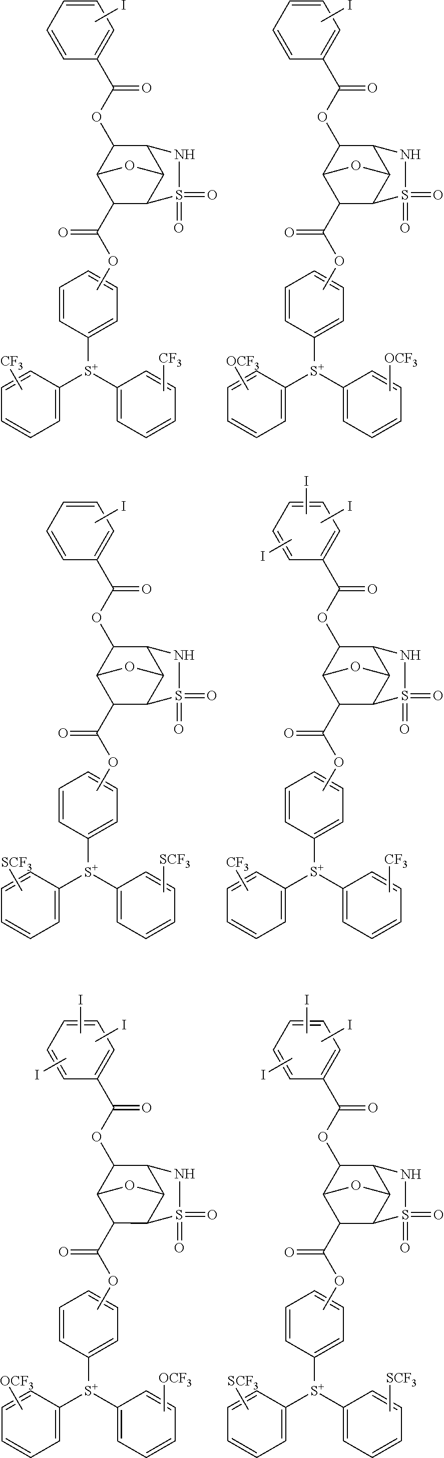

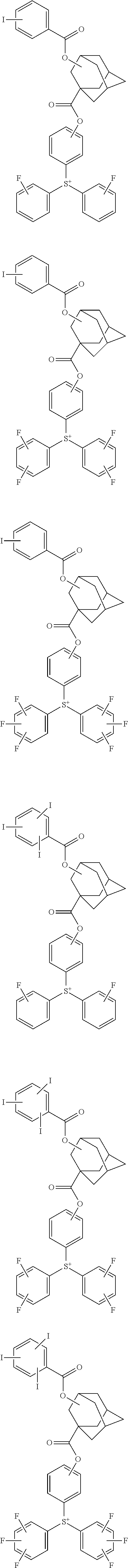

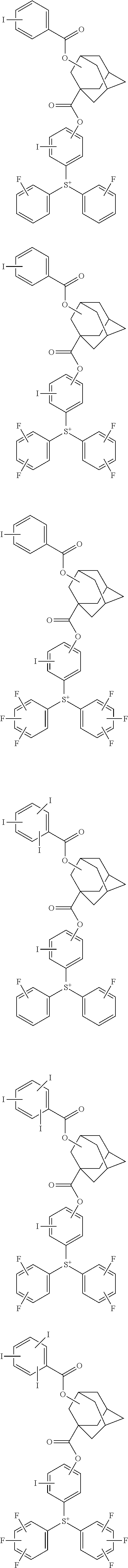

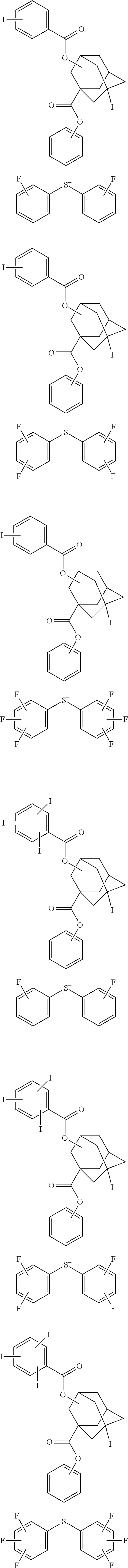

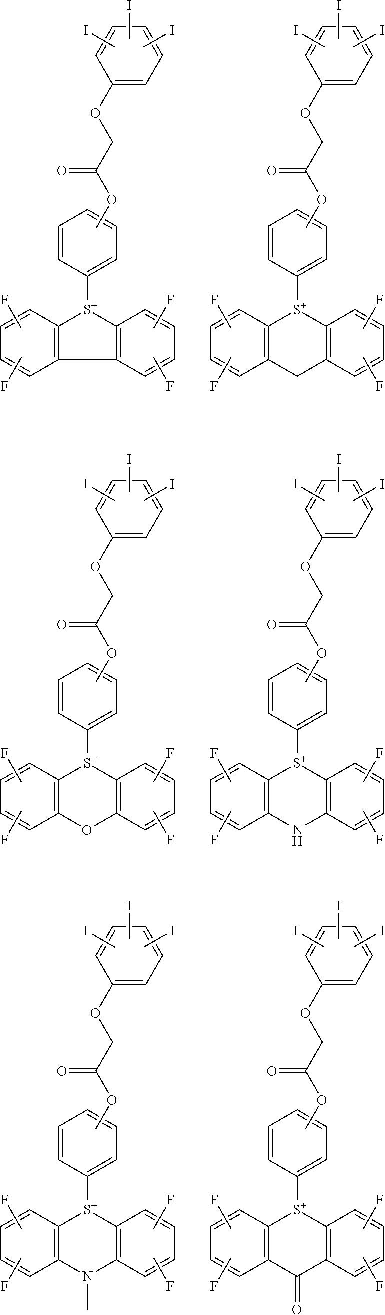

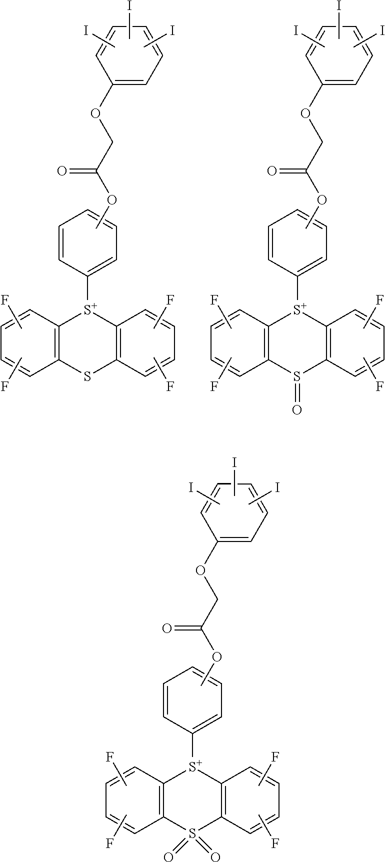

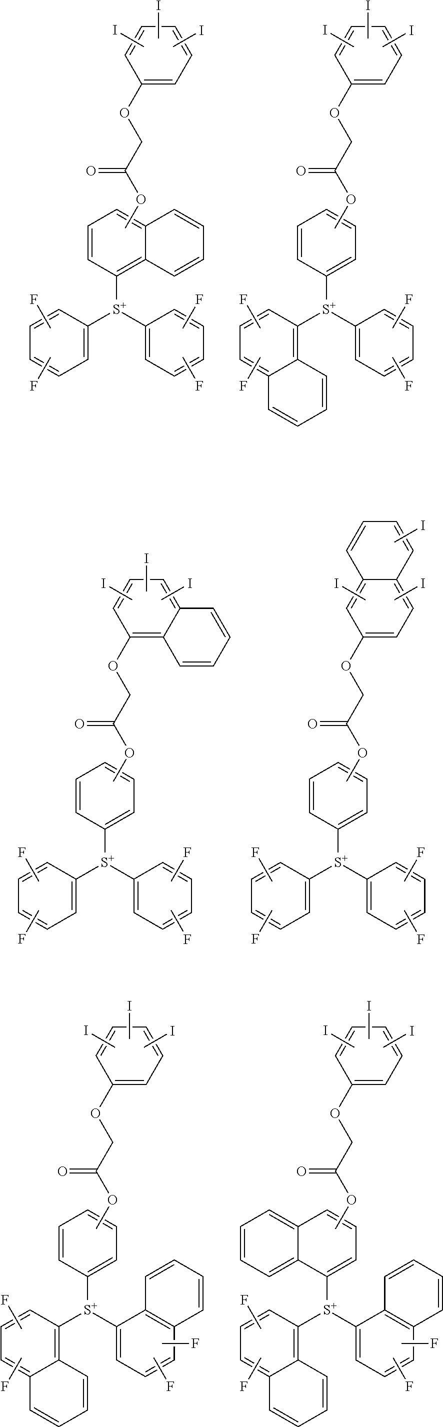

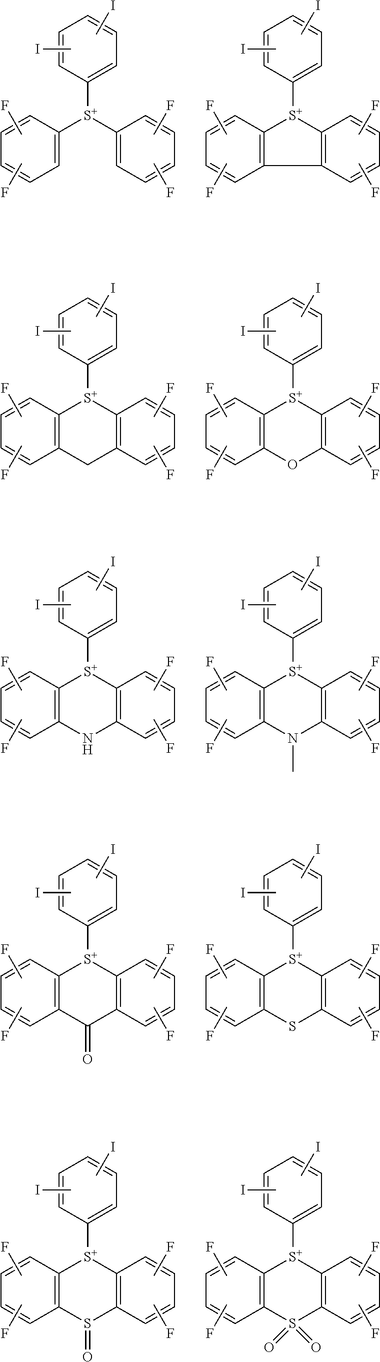

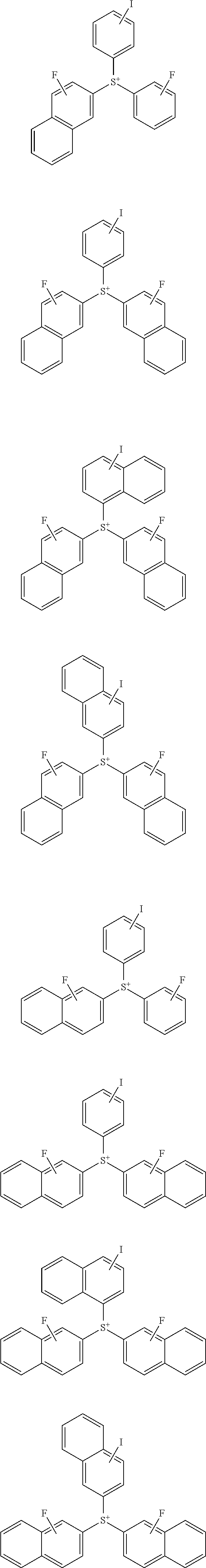

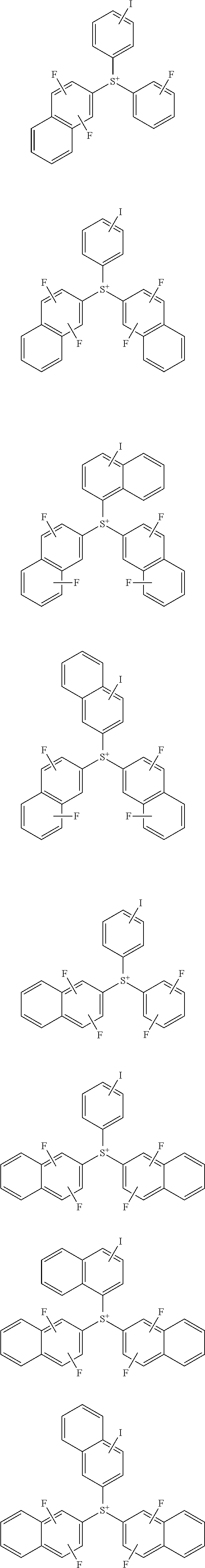

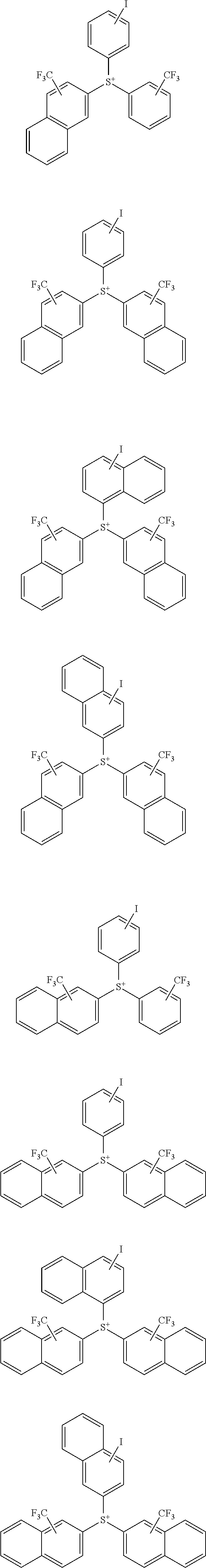

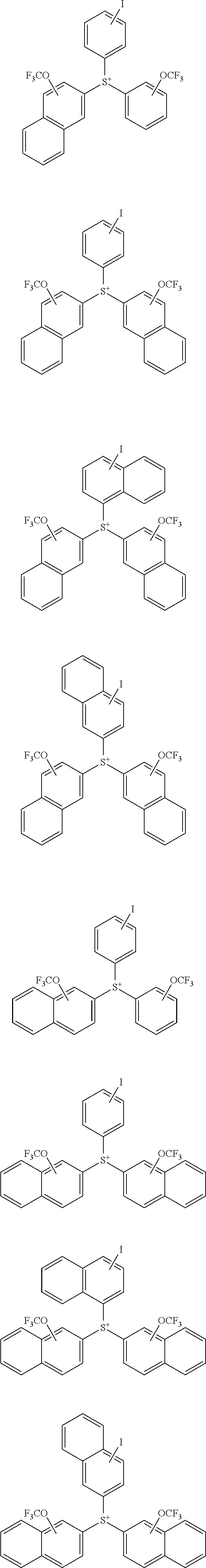

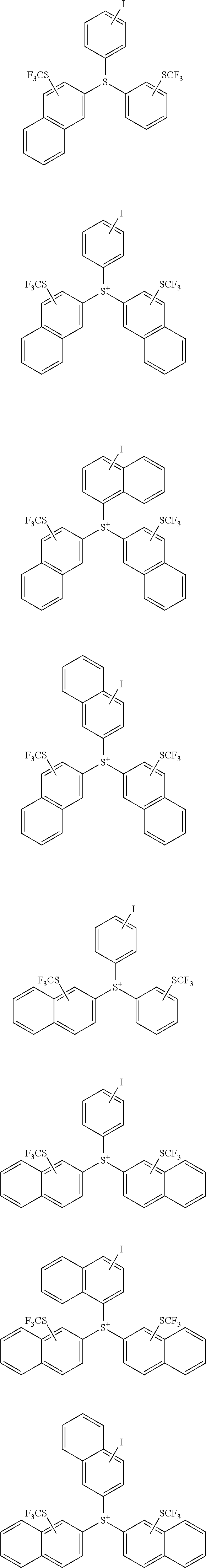

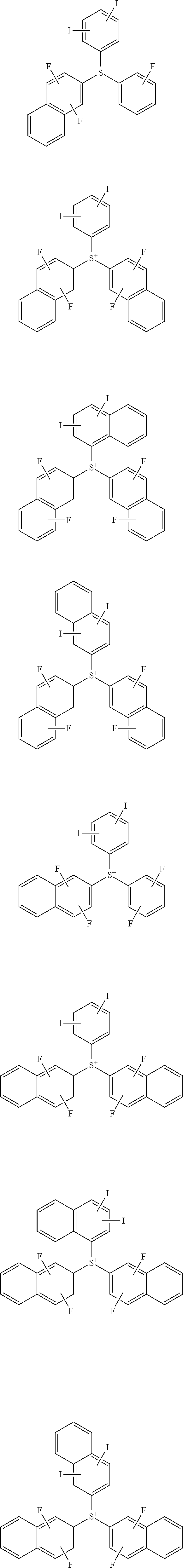

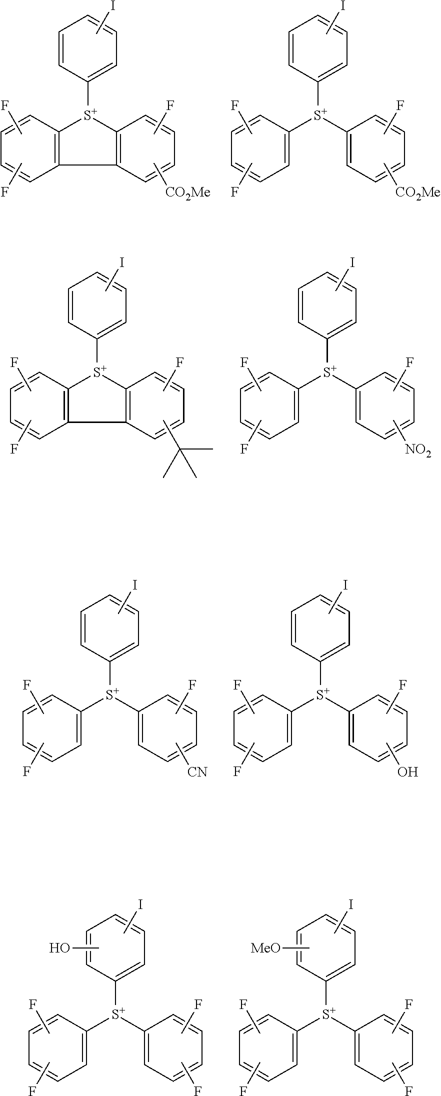

The polymer preferably further contains a repeat unit having the following formula (e) (hereinafter, also referred to as a repeat unit e).

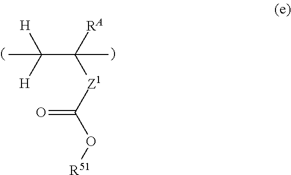

In the formula (e), R^{A} is a hydrogen atom, a fluorine atom, a methyl group, or a trifluoromethyl group. Z¹ is a single bond, a phenylene group, a naphthylene group, *—C(═O)—O—Z¹¹—, or *—C(═O)—N(H)—Z¹¹—, and the phenylene group or the naphthylene group may have a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom as a substituent. * represents a bond with a carbon atom of a main chain. Z¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring. R⁵¹ is a hydrogen atom or a C₁-C₂₀ group containing at least one structure selected from a hydroxy group other than a phenolic hydroxy group, a cyano group, a carbonyl group, a carboxy group, an ether bond, an ester bond, a sulfonate bond, a carbonate bond, a lactone ring, a sultone ring, and a carboxylic anhydride (—C(═O)—O—C(═O)—).

Specific examples of the repeat unit e include, but are not limited to, those listed below. Note that, in the following formulas, R^{A} is as defined above.

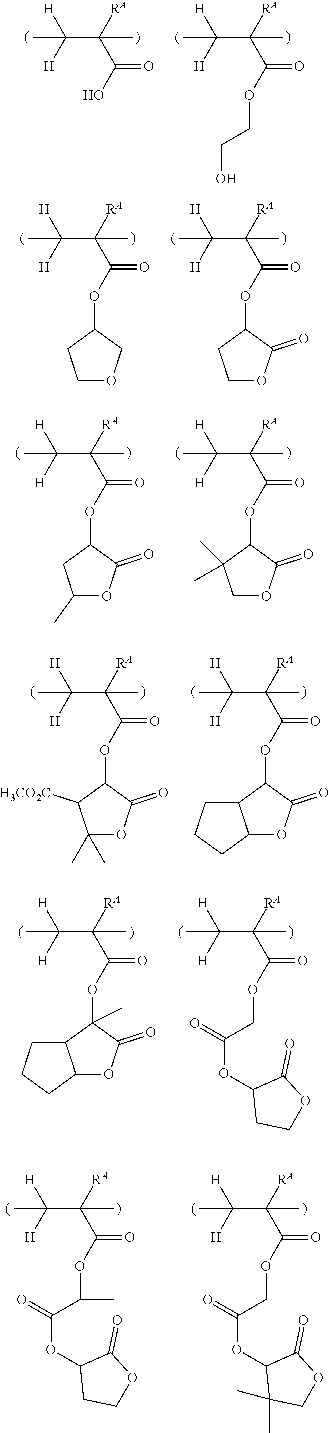

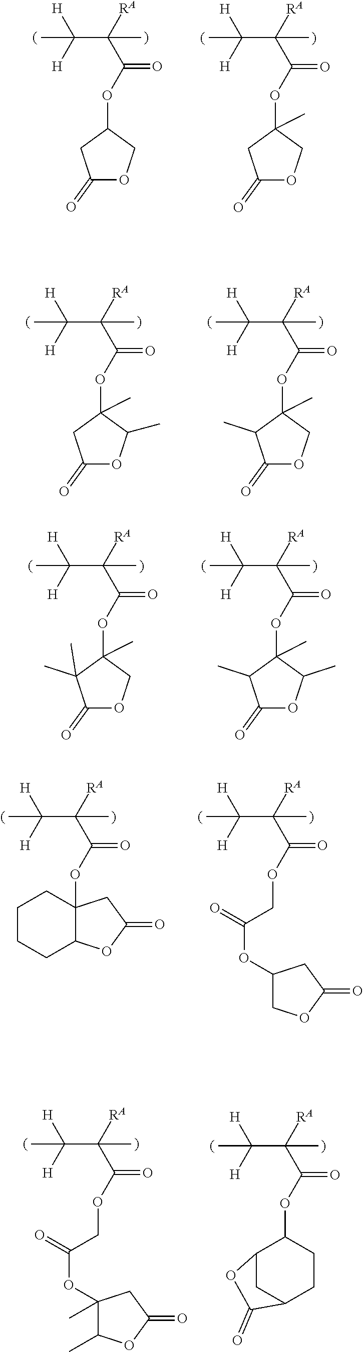

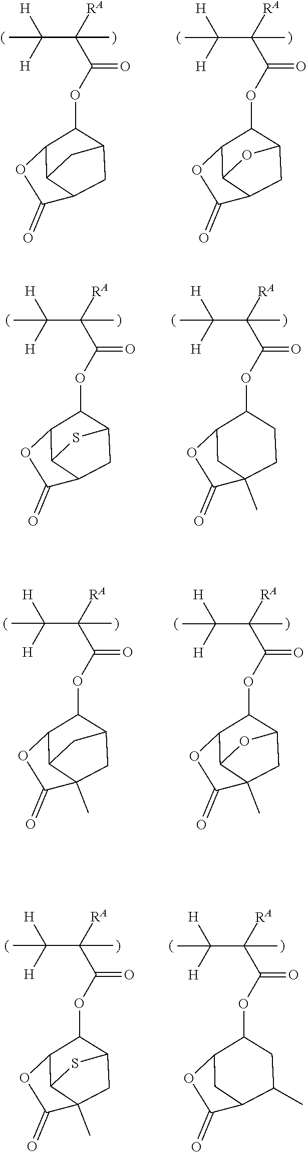

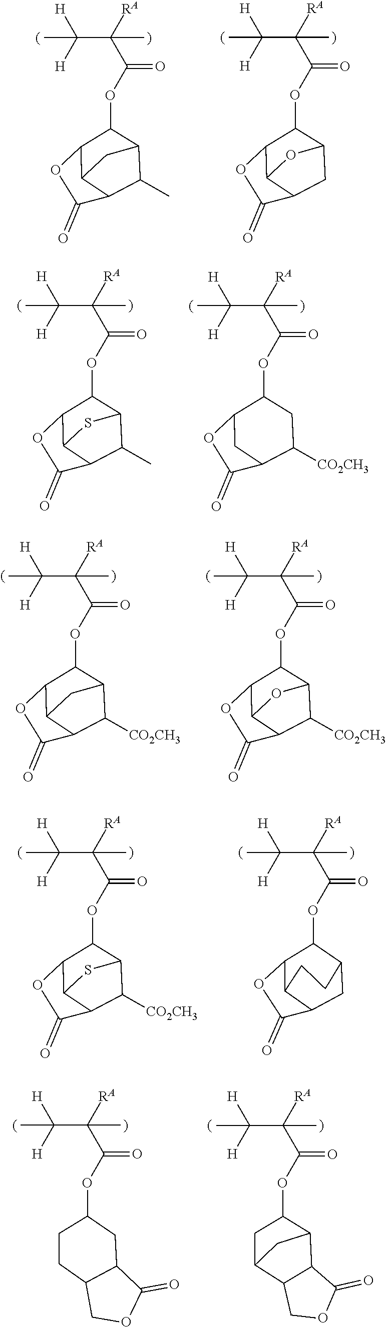

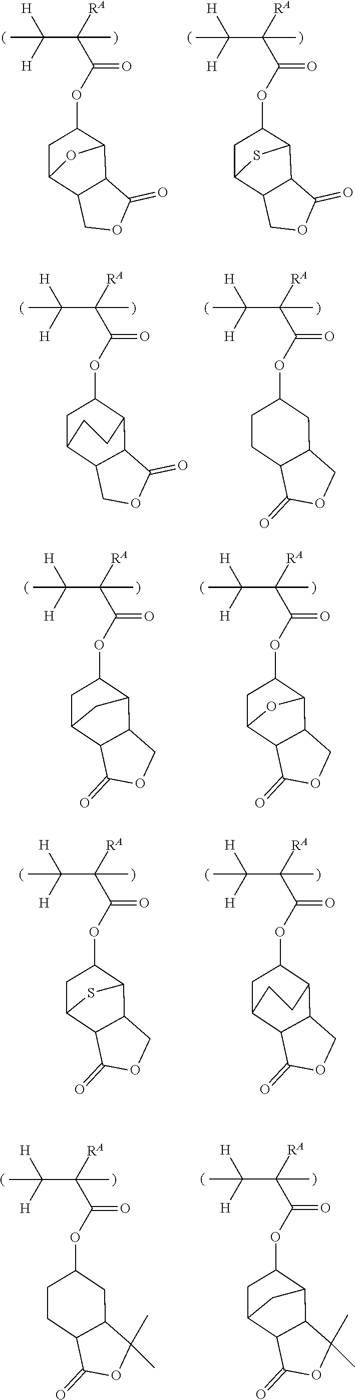

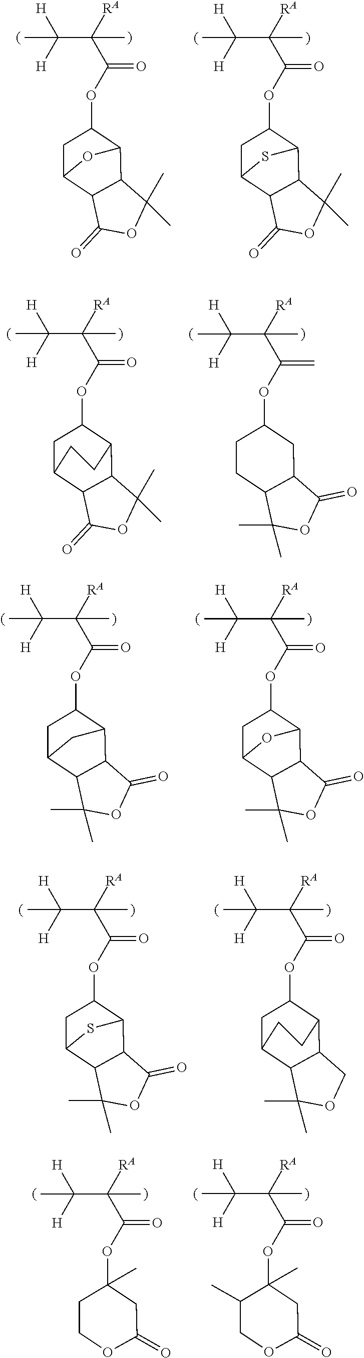

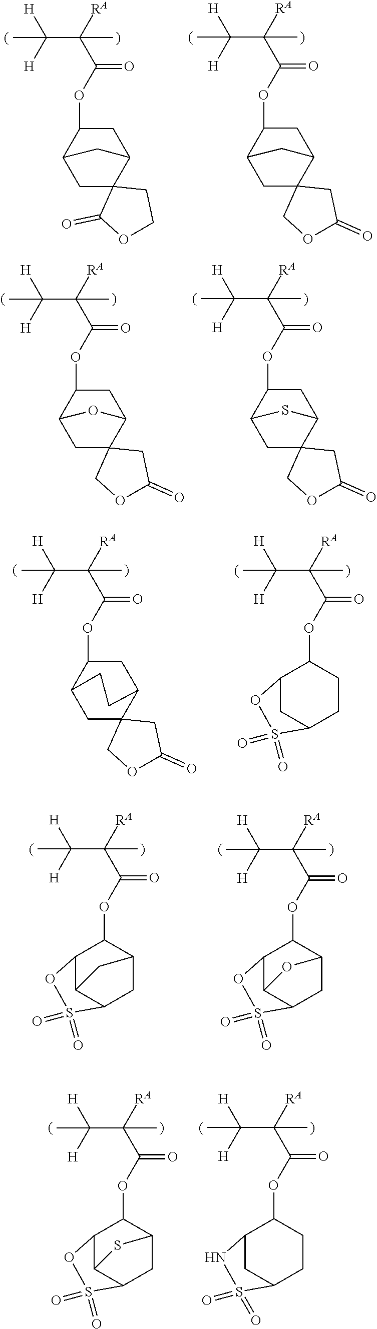

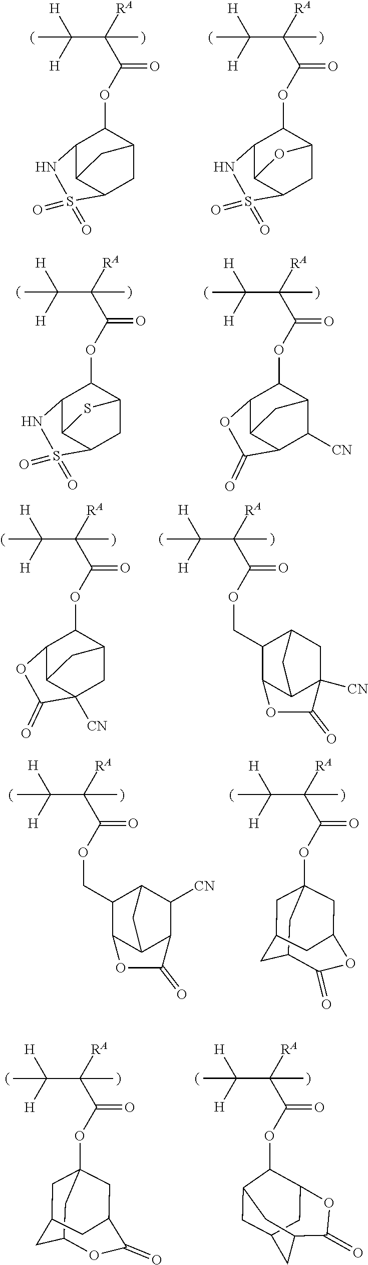

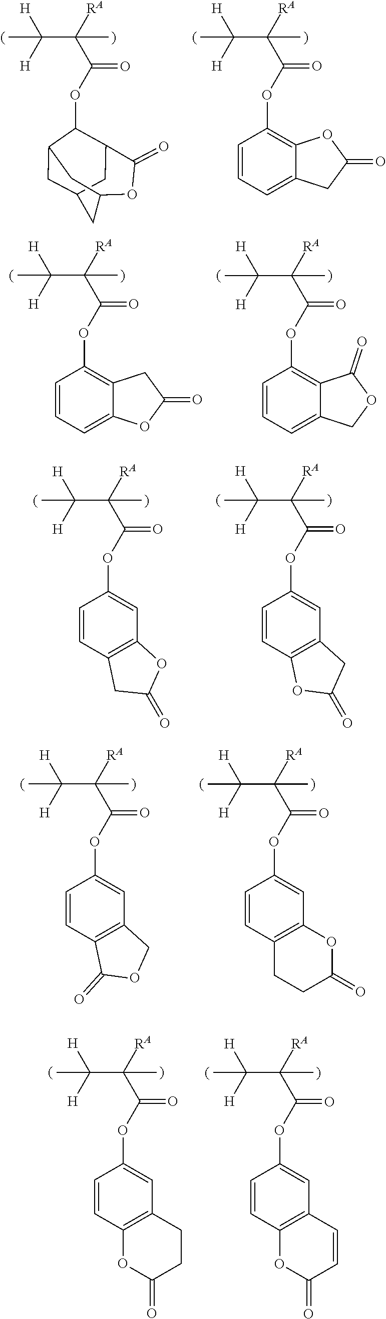

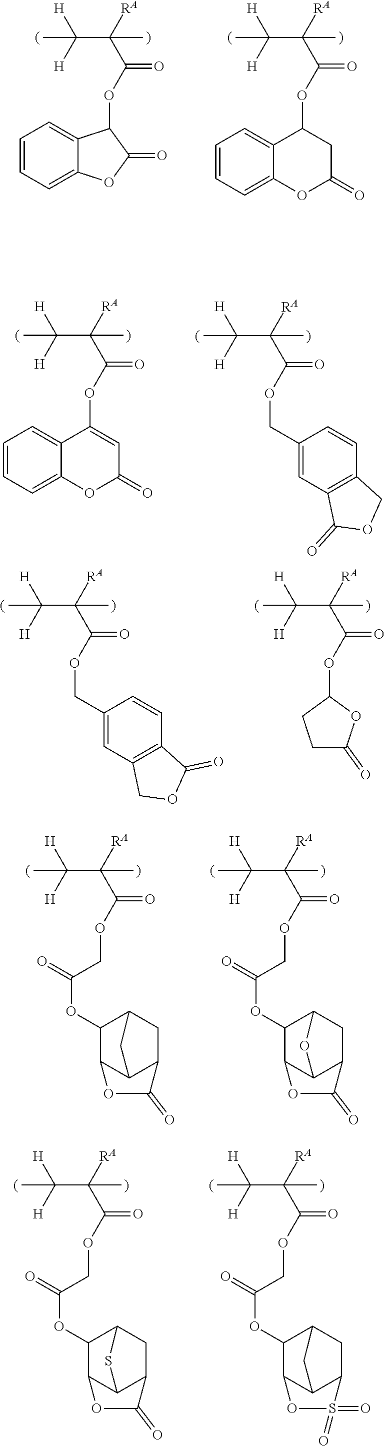

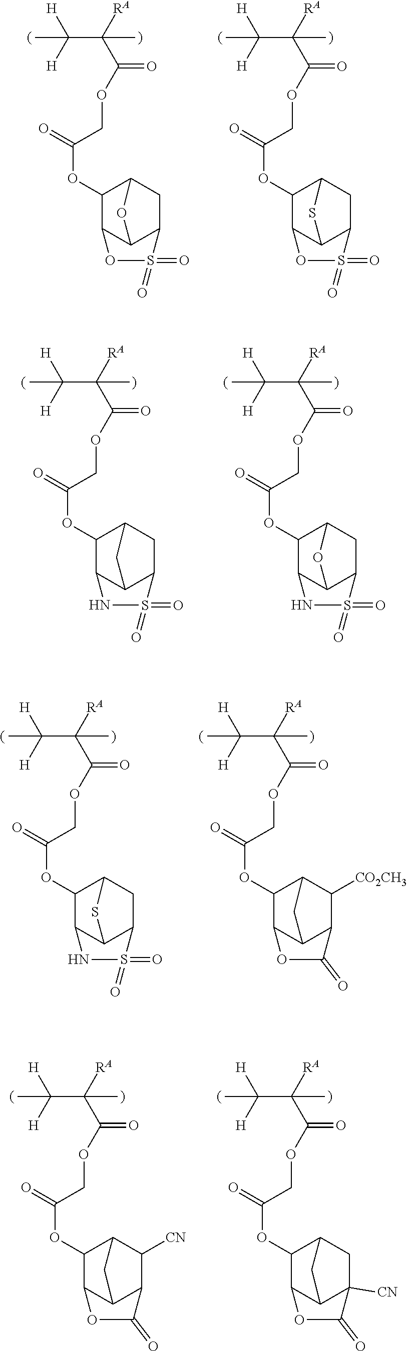

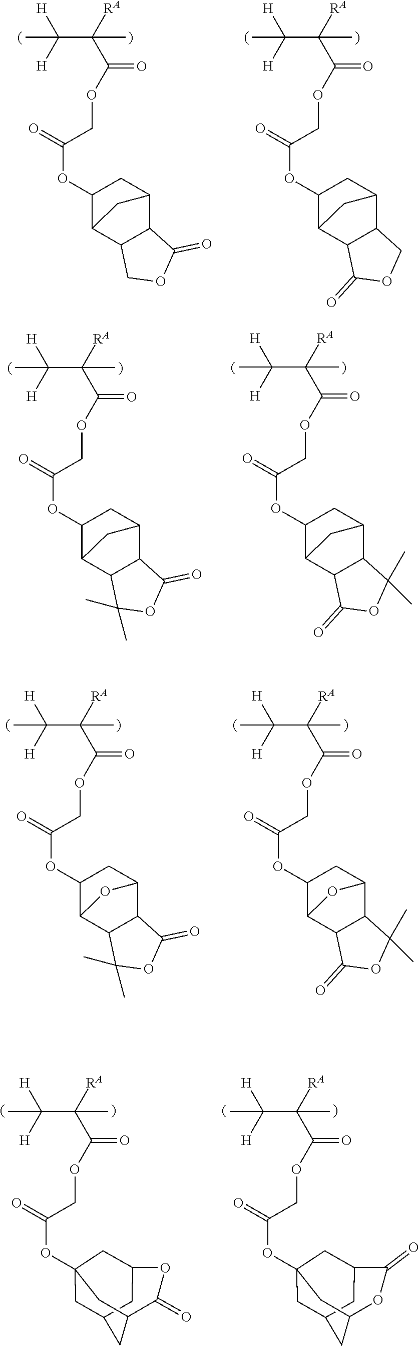

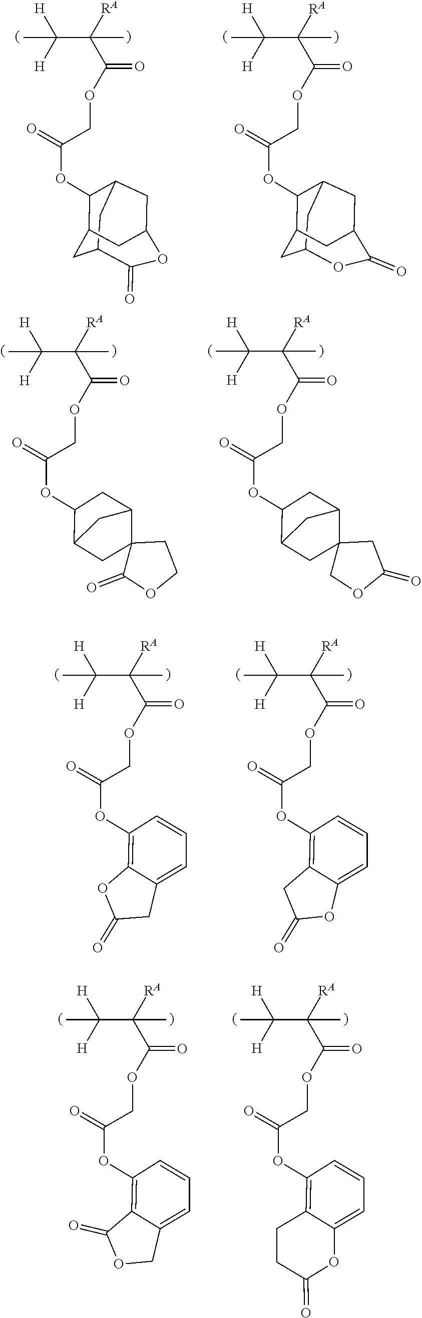

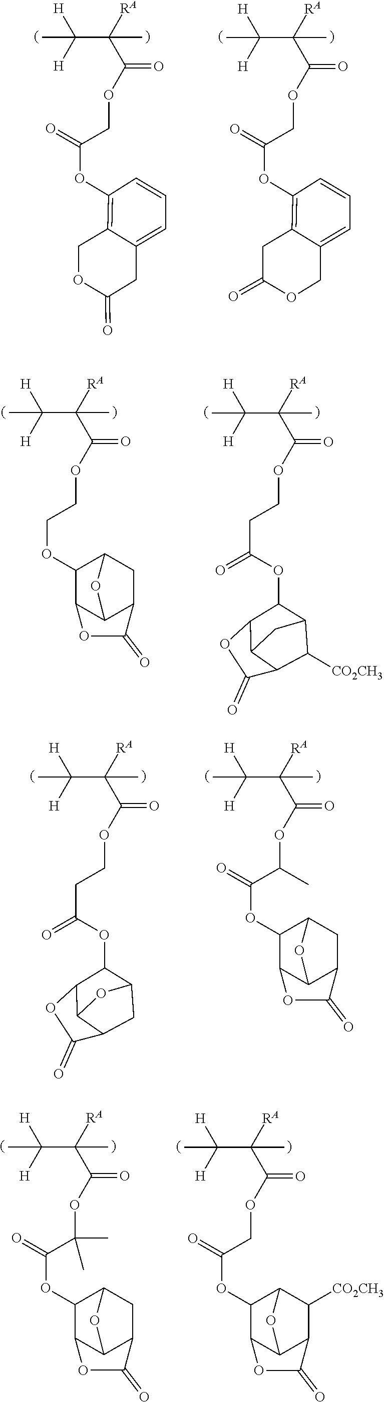

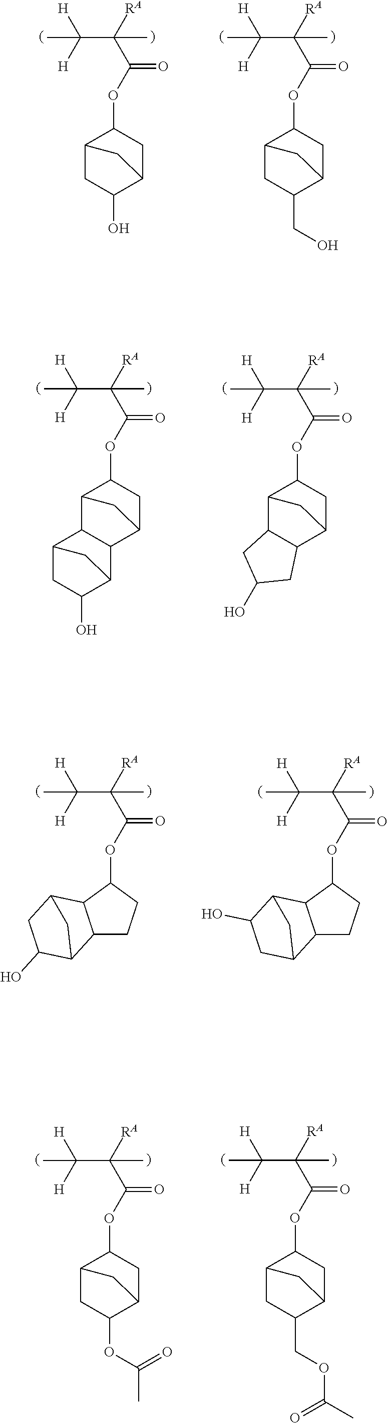

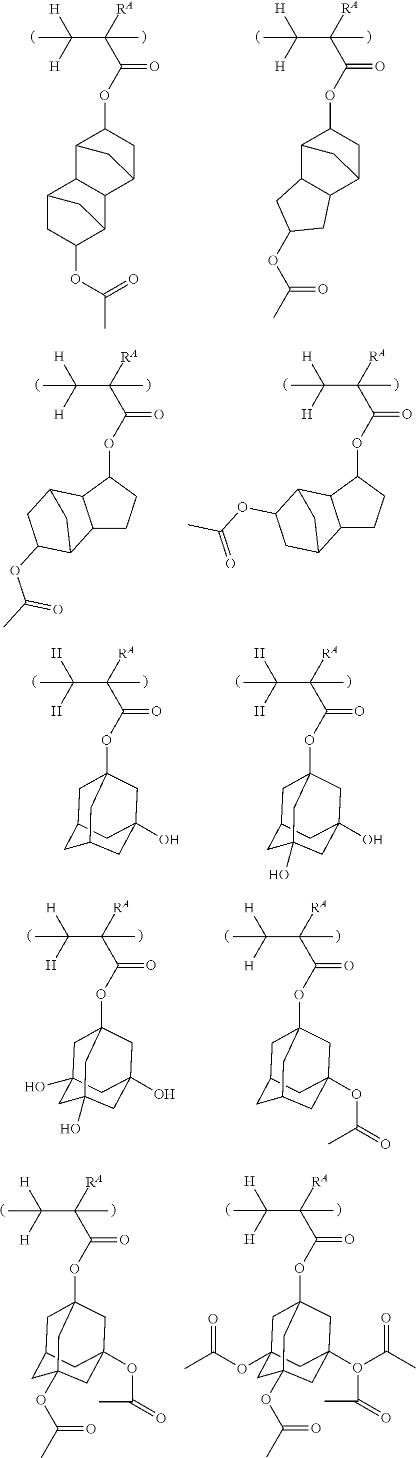

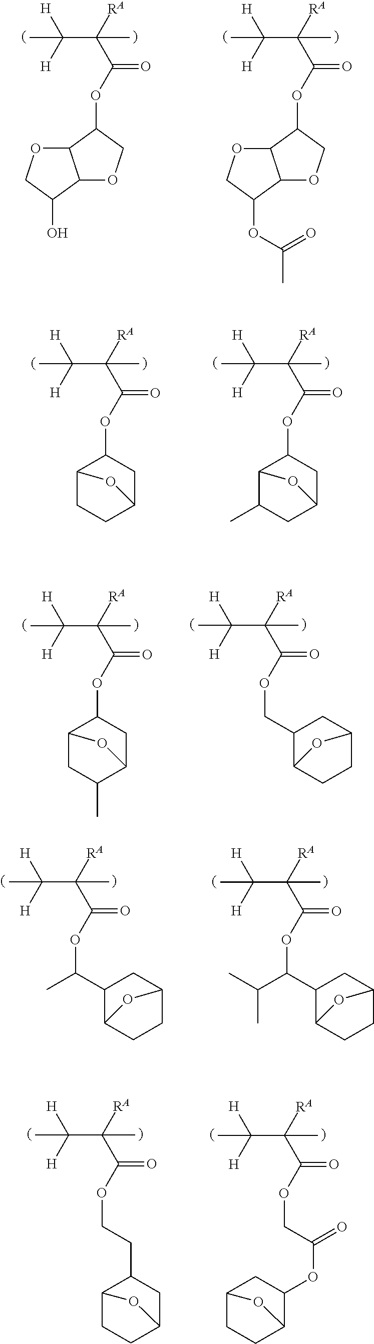

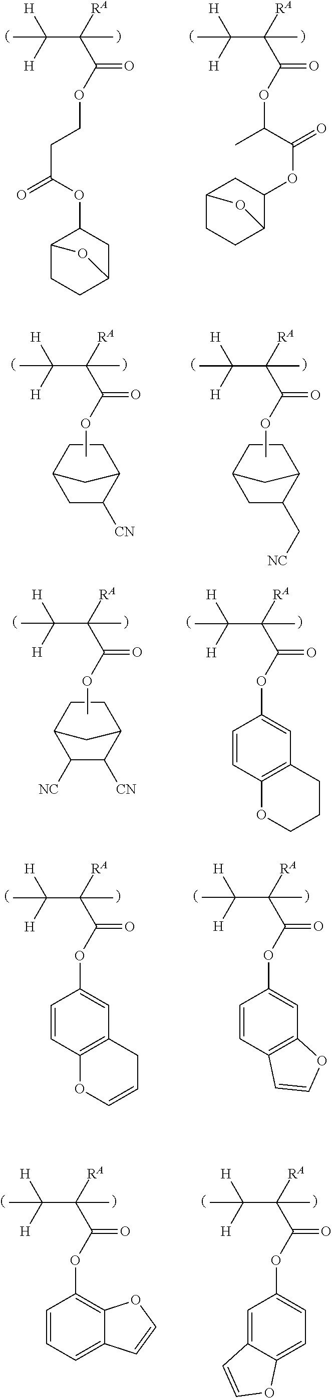

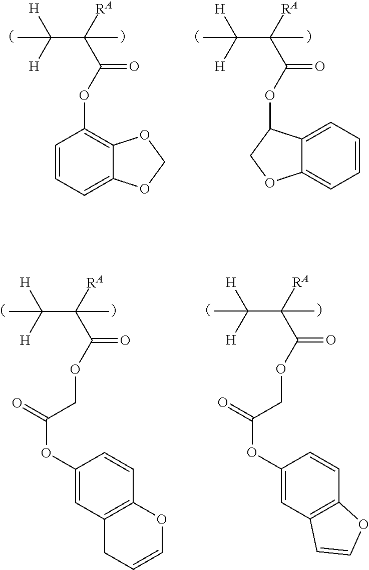

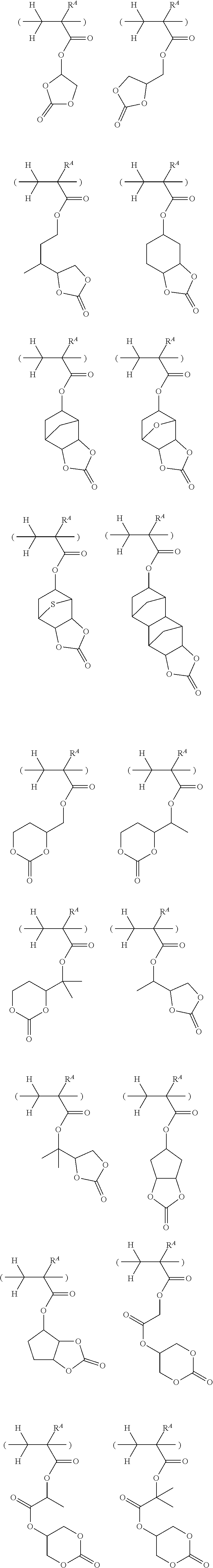

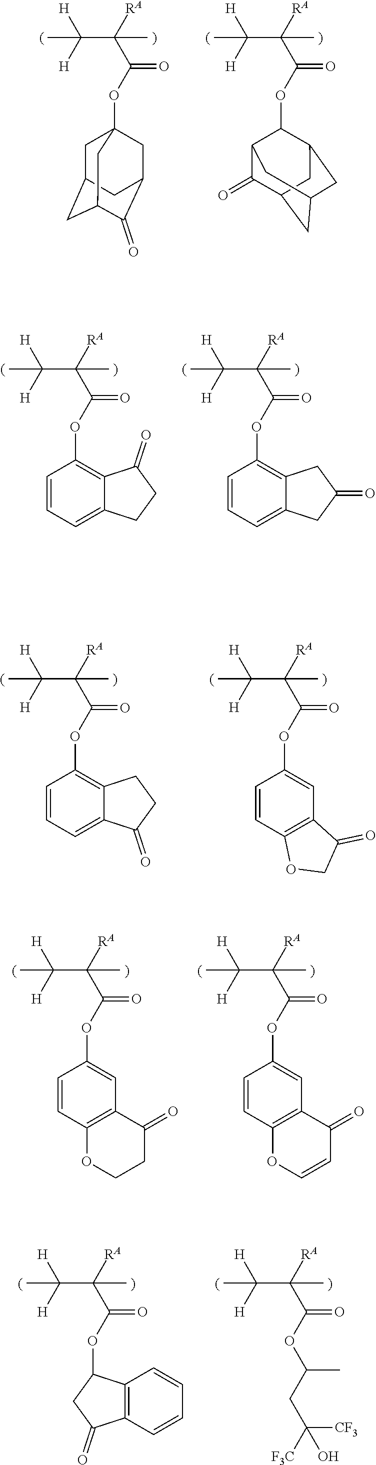

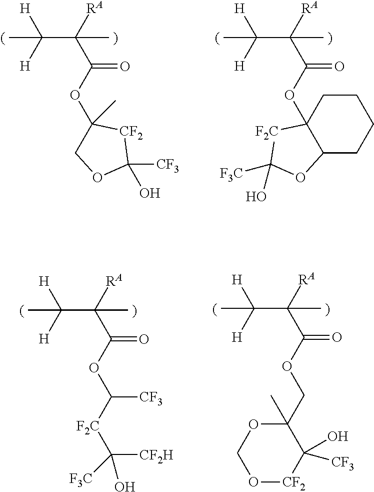

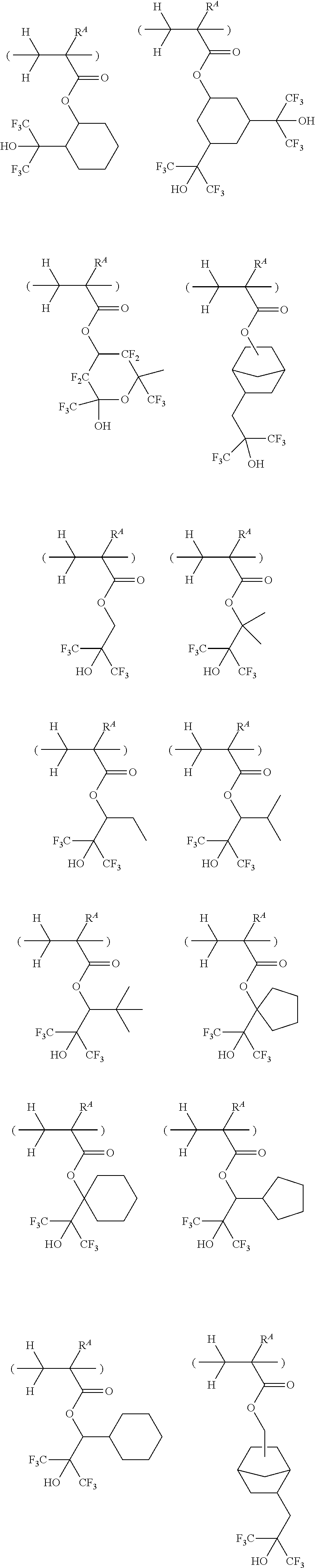

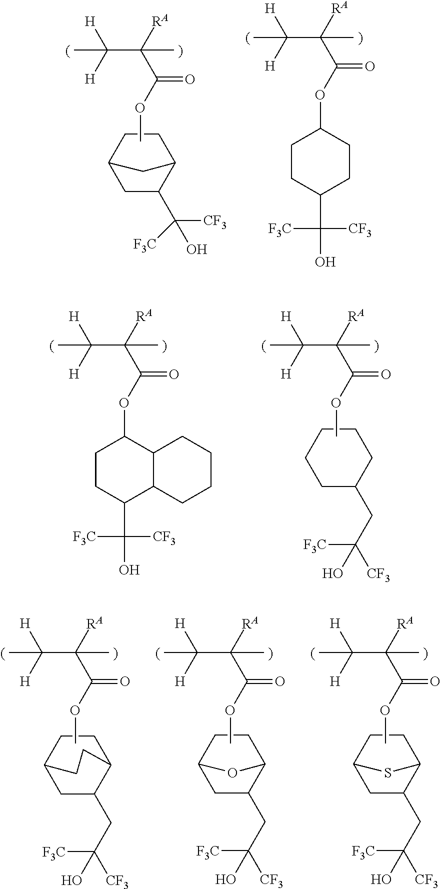

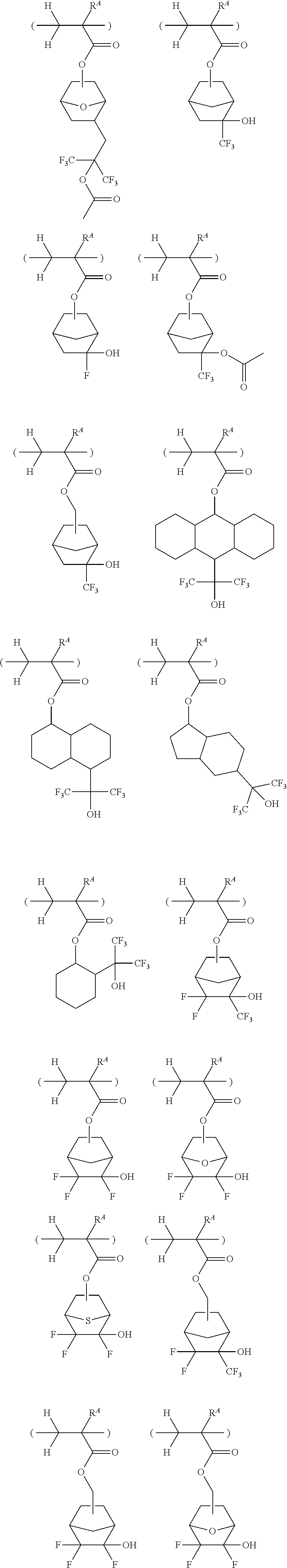

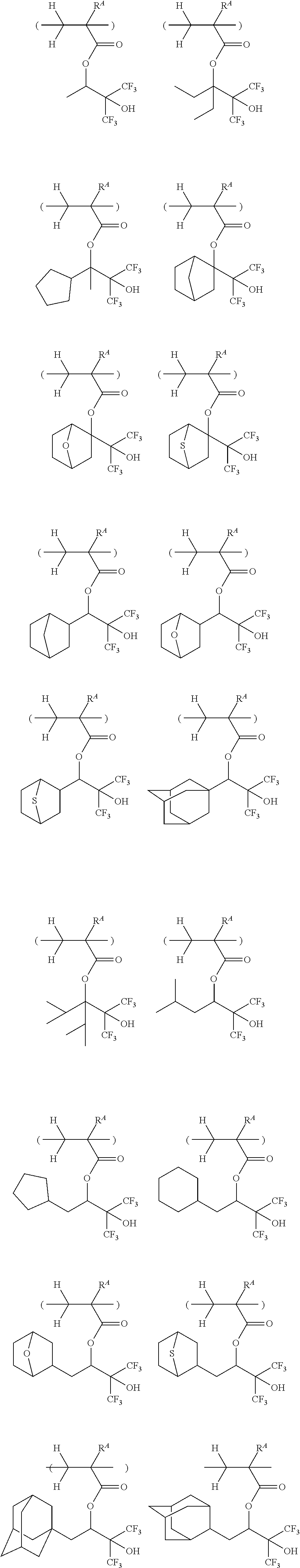

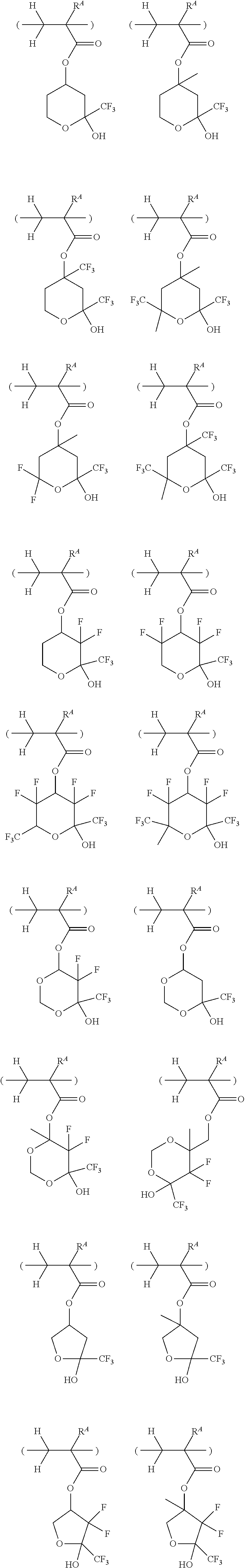

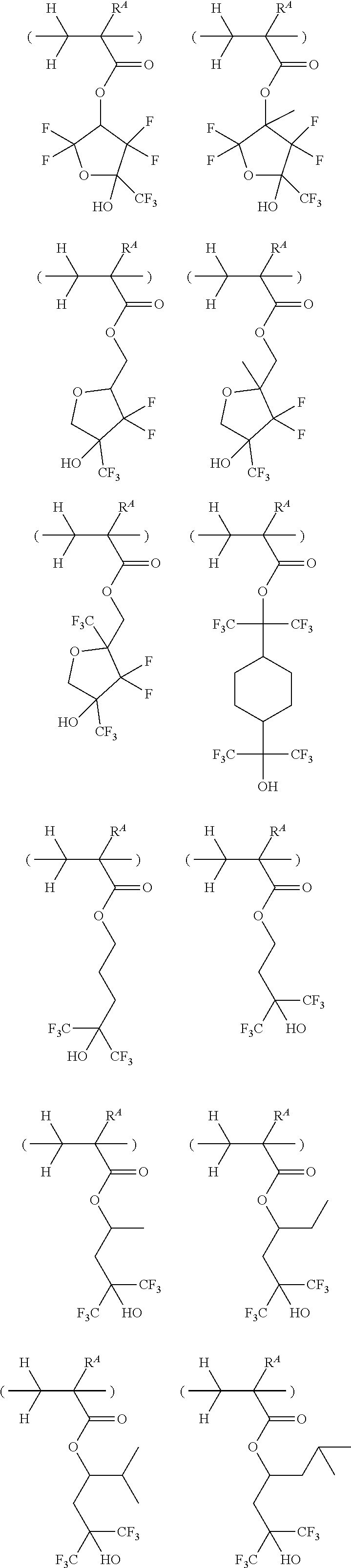

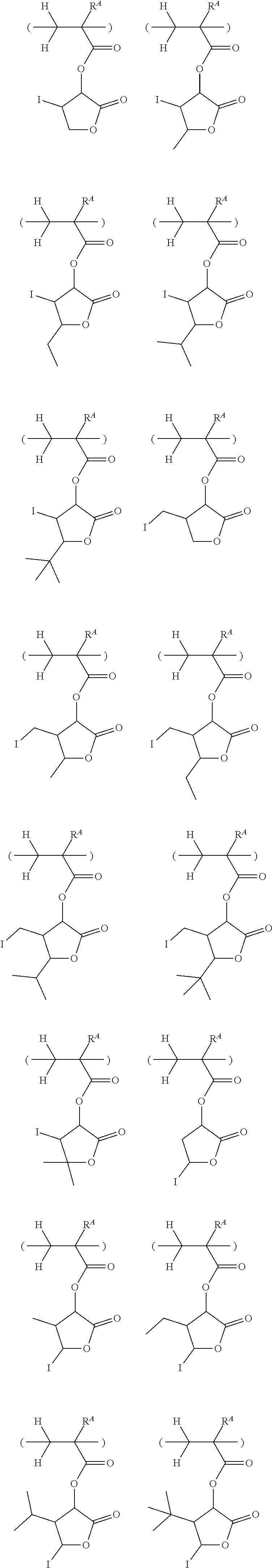

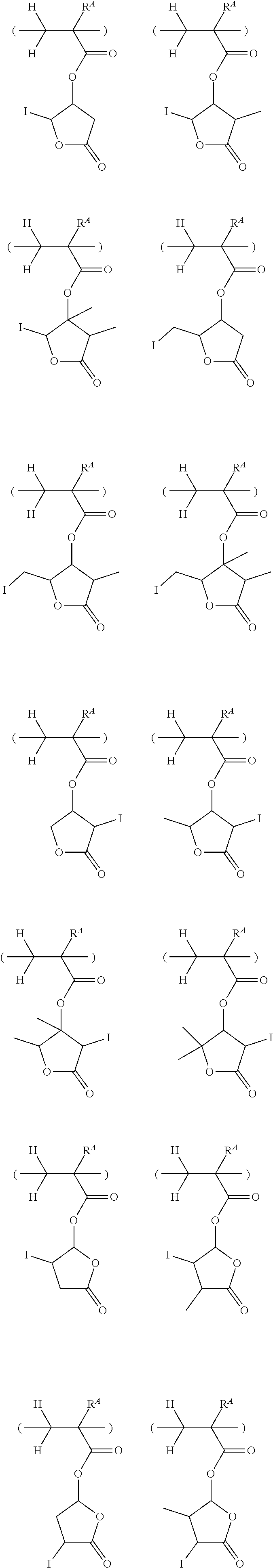

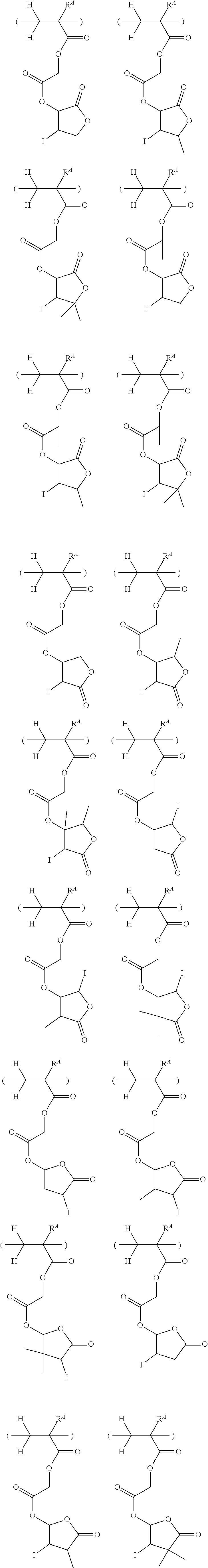

The repeat unit e is particularly preferably one having a lactone ring as a polar group in ArF lithography, and is particularly preferably one having a phenol moiety in KrF lithography, EB lithography, and EUV lithography.

The polymer may contain a repeat unit having a structure in which a hydroxy group is protected by an acid-unstable group (hereinafter, also referred to as a repeat unit f). The repeat unit e is not particularly limited as long as it has one or more structures in which a hydroxy group is protected, and a protecting group is decomposed by an action of an acid to generate a hydroxy group, but preferably has the following formula (f).

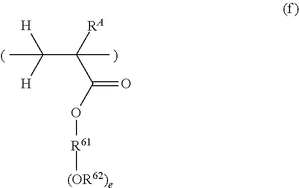

In the formula (f), R^{A} is a hydrogen atom, a fluorine atom, a methyl group, or a trifluoromethyl group. R⁶¹ is a (e+1)-valent C₁-C₃₀ hydrocarbon group which may contain a heteroatom. R⁶² is an acid-unstable group. e is 1, 2, 3, or 4.

In the formula (f), the acid-unstable group of R⁶² only needs to be a group that is deprotected by an action of an acid to generate a hydroxy group. A structure of R⁶² is not particularly limited, but is preferably an acetal structure, a ketal structure, a hydrocarbyloxycarbonyl group, a hydrocarbyloxymethyl group having the following formula (f1), or the like, and particularly preferably a hydrocarbyloxymethyl group having the following formula (f1).

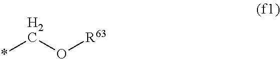

wherein * represents a bond, and R⁶³ is a C₁-C₁₅ hydrocarbyl group.

Specific examples of the acid-unstable group of R⁶², the hydrocarbyloxymethyl group of the formula (f1), and the repeat unit f include those similar to those exemplified in description of a repeat unit d described in JP-A 2020-111564.

The polymer may contain a repeat unit g derived from indene, benzofuran, benzothiophene, acenaphthylene, chromone, coumarin, norbornadiene, or a derivative thereof. Specific examples of a monomer that provides the repeat unit g include, but are not limited to, those listed below.

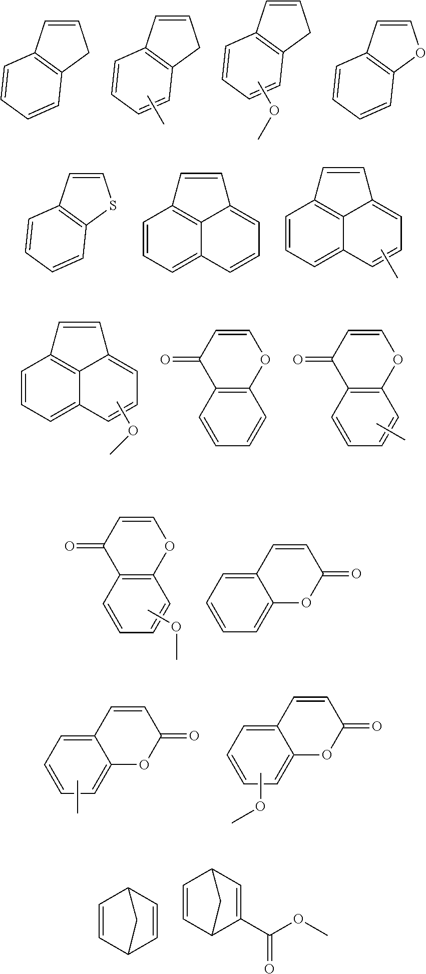

The polymer may contain a repeat unit h derived from styrene, indane, vinylpyridine, or vinylcarbazole.

In the polymer of the present invention, content ratios of the repeat units a, b1, b2, b3, c, d, e, f, g, and h are preferably 0<a≤0.4, 0<b1≤0.8, 0≤b2≤0.8, 0≤b3≤0.6, 0≤c≤0.6, 0≤d≤0.4, 0≤e≤0.6, 0≤f≤0.3, 0≤g≤0.3, and 0≤h≤0.3, and more preferably 0<a≤0.3, 0<b1≤0.7, 0≤b2≤0.7, 0≤b3≤0.5, 0≤c≤0.5, 0≤d≤0.3, 0≤e≤0.5, 0≤f≤0.2, 0≤g≤0.2, and 0≤h≤0.2. Provided that a+b1+b2+b3+c+d+e+f+g+h≤1.0.

A weight average molecular weight (Mw) of the polymer is preferably 1000 to 500,000, and more preferably 3000 to 100,000. When Mw is in this range, sufficient etching resistance is obtained, and there is no possibility of a decrease in resolution due to inability to ensure a difference in dissolution rate between before and after exposure. Note that, in the present invention, Mw is a value measured in terms of polystyrene by gel permeation chromatography (GPC) using tetrahydrofuran (THF) or N,N-dimethylformamide (DMF) as a solvent.

In a molecular weight distribution (Mw/Mn) of the polymer, an influence of Mw/Mn tends to increase as a pattern rule is finer. Therefore, in order to obtain a resist composition suitably used for fine pattern dimensions, Mw/Mn preferably has a narrow dispersion value of 1.0 to 2.0. Within the above range, the amount of a polymer having a low molecular weight or a high molecular weight is small, and there is no possibility that a foreign matter is observed on a pattern or the shape of the pattern is deteriorated after exposure.

Examples of a method for synthesizing the polymer include a method in which a monomer that provides the above-described repeat unit is added to an organic solvent with a radical polymerization initiator and heated to be polymerized.

Specific examples of the organic solvent used during polymerization include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL). Specific examples of the polymerization initiator include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The addition amount of these initiators is preferably 0.01 to 25 mol % based on the total moles of monomers to be polymerized. A reaction temperature is preferably 50 to 150° C., and more preferably 60 to 100° C. A reaction time is preferably 2 to 24 hours, and more preferably 2 to 12 hours from a viewpoint of production efficiency.

The polymerization initiator may be added to the monomer solution, and the mixture may be supplied to a reaction vessel, or an initiator solution may be prepared separately from the monomer solution, and the initiator solution and the monomer solution may be each independently supplied to the reaction vessel. The monomer solution and the initiator solution are preferably each independently prepared and dropwise added from a viewpoint of quality control because there is a possibility that a polymerization reaction proceeds by radicals generated from an initiator during a standby time to generate an ultrahigh polymer. An acid-unstable group introduced into the monomer may be used as it is, or protection or partial protection may be performed after polymerization. In order to adjust a molecular weight, a known chain transfer agent such as dodecyl mercaptan or 2-mercaptoethanol may be used in combination. In this case, the addition amount of these chain transfer agents is preferably 0.01 to 20 mol % based on the total moles of monomers to be polymerized.

In a case of a monomer containing a hydroxy group, the hydroxy group may be replaced with an acetal group such as ethoxyethoxy, which can be easily deprotected by an acid during polymerization, and then deprotected by a weak acid and water after polymerization, or may be replaced with an acetyl group, a formyl group, a pivaloyl group, or the like, and then subjected to alkali hydrolysis after polymerization.

When hydroxystyrene or hydroxyvinylnaphthalene is copolymerized, hydroxystyrene or hydroxyvinylnaphthalene and another monomer may be heated to be polymerized by adding a radical polymerization initiator in an organic solvent, but acetoxystyrene or acetoxyvinylnaphthalene may be used, and an acetoxy group may be deprotected by alkali hydrolysis after polymerization to obtain polyhydroxystyrene or hydroxypolyvinylnaphthalene.

Specific examples of a base during alkali hydrolysis include ammonia water and triethylamine. A reaction temperature is preferably-20 to 100° C., and more preferably 0 to 60° C. A reaction time is preferably 0.2 to 100 hours, and more preferably 0.5 to 20 hours.

Note that the amount of each monomer in the monomer solution only needs to be appropriately set, for example, so as to have the above-described preferable content ratios of the repeat units.

Regarding a polymer obtained by the manufacturing method, a reaction solution obtained by the polymerization reaction may be used as a final product, or a powder obtained through a purification step such as a reprecipitation method in which a polymerization liquid is added to a poor solvent to obtain a powder may be used as a final product. However, it is preferable to use a polymer solution obtained by dissolving a powder obtained by the purification step in a solvent as a final product from a viewpoint of operation efficiency and quality stabilization.

Specific examples of the solvent used at this time include: ketones such as cyclohexanone and methyl-2-n-pentyl ketone; alcohols such as 3-methoxybutanol, 3-methyl-3-methoxybutanol, 1-methoxy-2-propanol, and 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono tert-butyl ether acetate; lactones such as GBL; alcohols such as diacetone alcohol (DAA); high boiling point alcohol solvents such as diethylene glycol, propylene glycol, glycerin, 1,4-butanediol, and 1,3-butanediol; and a mixed solvent thereof, described in paragraphs [0144] to [0145] of JP-A 2008-111103.

A concentration of the polymer in the polymer solution is preferably 0.01 to 30 wt %, and more preferably 0.1 to 20 wt %.

The reaction solution and the polymer solution are preferably subjected to filter filtration. By performing filter filtration, foreign matters and gels that may cause defects can be removed, and it is effective in terms of quality stabilization.

Examples of a material of the filter used for the filter filtration include a fluorocarbon-based material, a cellulose-based material, a nylon-based material, a polyester-based material, and a hydrocarbon-based material. In a step of filtering a resist composition, a filter formed of a fluorocarbon-based material called Teflon®, a hydrocarbon-based material such as polyethylene or polypropylene, or nylon is preferable. A pore size of the filter can be appropriately selected according to a target cleanliness, but is preferably 100 nm or less, and more preferably 20 nm or less. These filters may be used singly or in combination of two or more types thereof. In a filtration method, a solution may be caused to pass only once, but it is more preferable to perform filtration a plurality of times by circulating the solution. The filtration step can be performed in any order and any number of times in the polymer manufacturing process, but it is preferable to filter the reaction solution after a polymerization reaction, the polymer solution, or both.

### [Chemically Amplified Resist Composition]

### (A) Base Polymer

A chemically amplified resist composition of the present invention contains a base polymer containing the above-described polymer as a component (A).

The polymer may be used singly, or two or more polymers having different composition ratios and Mw and/or Mw/Mn may be used in combination. In addition, the base polymer (A) may contain a hydrogenated ring-opened metathesis polymer in addition to the polymer, and as the hydrogenated ring-opened metathesis polymer, those described in JP-A 2003-66612 can be used.

### (B) Organic Solvent

The chemically amplified resist composition of the present invention may contain an organic solvent as a component (B). The organic solvent (B) is not particularly limited as long as it can dissolve the components described above and components described below. Specific examples of such an organic solvent include: ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol; ketoalcohols such as DAA; ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono tert-butyl ether acetate; lactones such as GBL; and a mixed solvent thereof.

Among these organic solvents, 1-ethoxy-2-propanol, PGMEA, cyclohexanone, GBL, ethyl lactate, DAA, and a mixed solvent thereof, in which solubility of the base polymer as the component (A) is particularly excellent, are preferable.

The content of the organic solvent (B) in the chemically amplified resist composition of the present invention is preferably 200 to 5000 parts by weight, and more preferably 400 to 3500 parts by weight per 80 parts by weight of the base polymer (A). The organic solvents (B) may be used singly or in combination of two or more types thereof.

### (C) Quencher

The chemically amplified resist composition of the present invention may contain a quencher as a component (C). Note that, in the present invention, the quencher is a material for forming a desired pattern by trapping a strong acid generated from a photoacid generator in a chemically amplified resist composition to prevent diffusion to an unexposed portion. Note that the strong acid means an acid having an acidity sufficient to cause a deprotection reaction of an acid-unstable group.

Specific examples of the quencher (C) include an onium salt having the following formula (1) or (2):

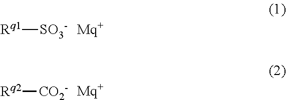

In the formula (1), R^{q1} is a hydrogen atom or a C₁-C₄₀ hydrocarbyl group which may contain a heteroatom, except a hydrocarbyl group in which a hydrogen atom bonded to a carbon atom at an α-position of a sulfo group is replaced with a fluorine atom or a fluoroalkyl group. In the formula (2), R^{q2} is a hydrogen atom or a C₁-C₄₀ hydrocarbyl group which may contain a heteroatom.

Specific examples of the C₁-C₄₀ hydrocarbyl group of R⁹¹ include: a C₁-C₄₀ alkyl group such as a methyl group, an ethyl group, a n-propyl group, an isopropyl group, a n-butyl group, an isobutyl group, a sec-butyl group, a tert-butyl group, a n-pentyl group, a tert-pentyl group, a n-hexyl group, a n-octyl group, a 2-ethylhexyl group, a n-nonyl group, or a n-decyl group; a C₃-C₄₀ cyclic saturated hydrocarbyl group such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}] decyl group, or an adamantyl group; and a C₆-C₄₀ aryl group such as a phenyl group, a naphthyl group, or an anthracenyl group. Some or all of hydrogen atoms of the hydrocarbyl group may be replaced with a group containing a heteroatom such as an oxygen atom, a sulfur atom, a nitrogen atom, or a halogen atom, some of (—CH₂—)s in the hydrocarbyl group may be replaced with a group containing a heteroatom such as an oxygen atom, a sulfur atom, or a nitrogen atom, and as a result, the hydrocarbyl group may contain a hydroxy group, a fluorine atom, a chlorine atom, a bromine atom, an iodine atom, a cyano group, a carbonyl group, an ether bond, an ester bond, a sulfonate bond, a carbonate bond, a lactone ring, a sultone ring, a carboxylic acid anhydride (—C(═O)—O—C(—O)—), a haloalkyl group, or the like.

Specific examples of the hydrocarbyl group of R^{q2} include, in addition to the substituents exemplified as specific examples of R^{q1}, a fluorinated saturated hydrocarbyl group such as a trifluoromethyl group or a trifluoroethyl group, and a fluorinated aryl group such as a pentafluorophenyl group or a 4-trifluoromethylphenyl group.

Specific examples of an anion of the onium salt having the formula (1) include, but are not limited to, those listed below.

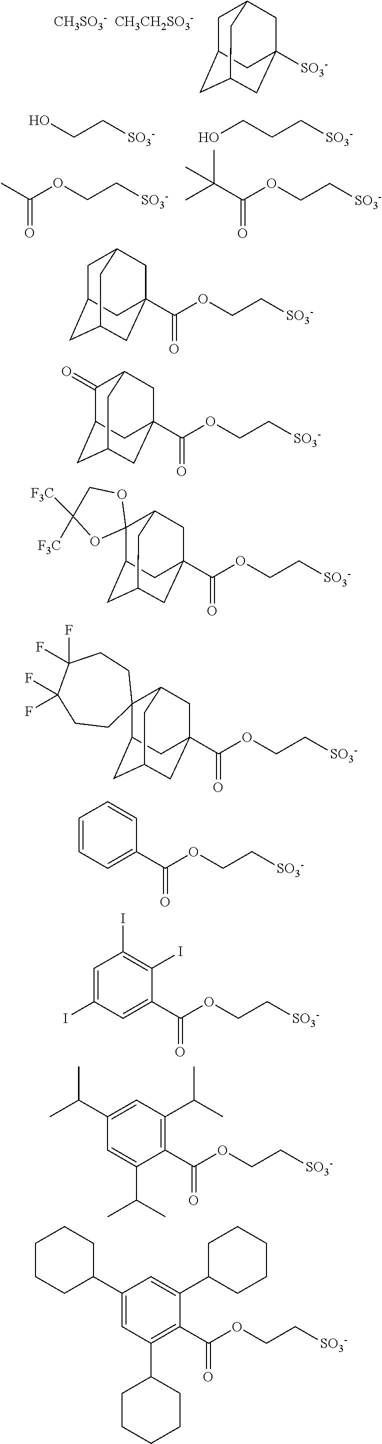

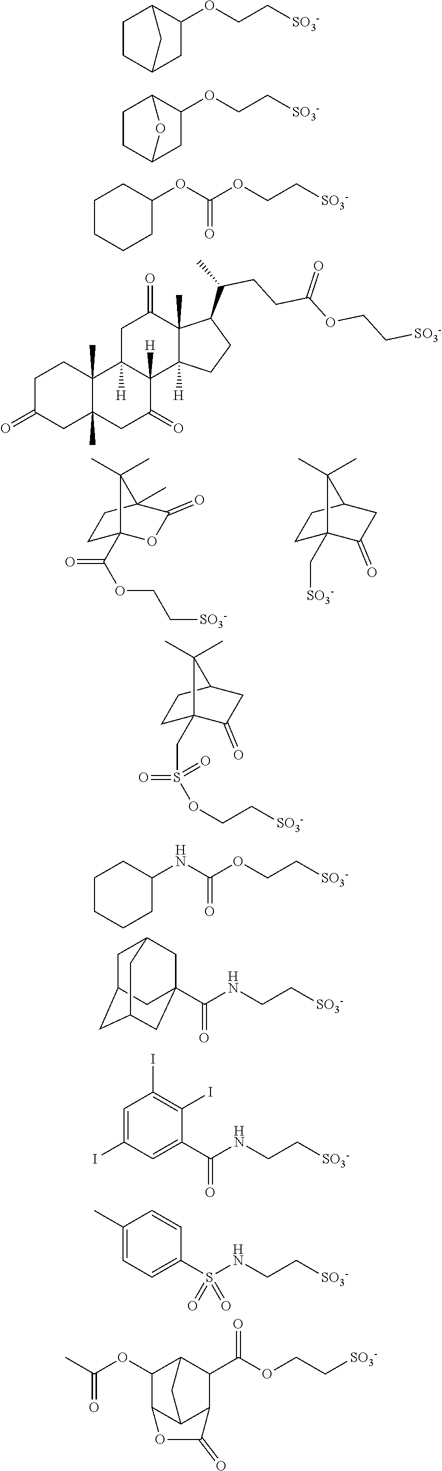

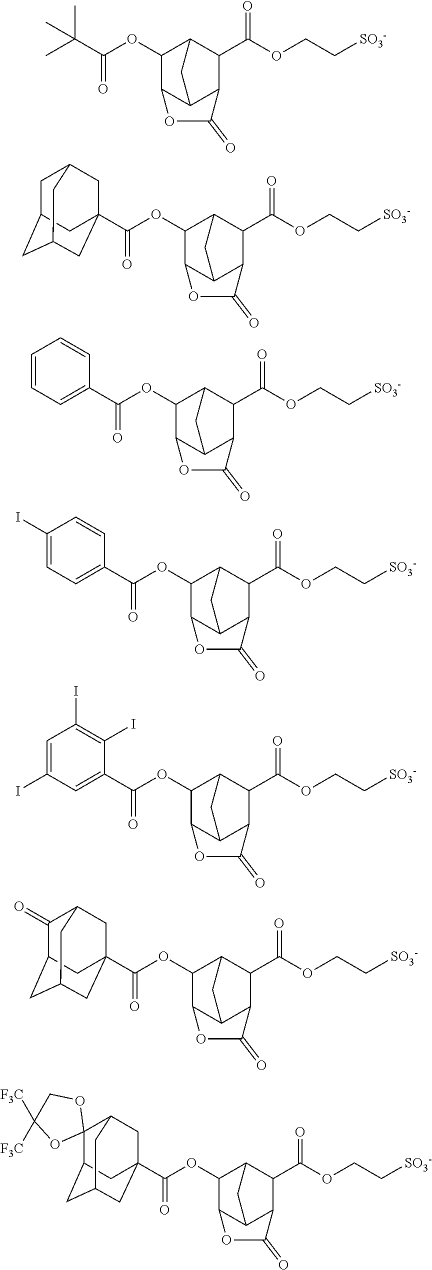

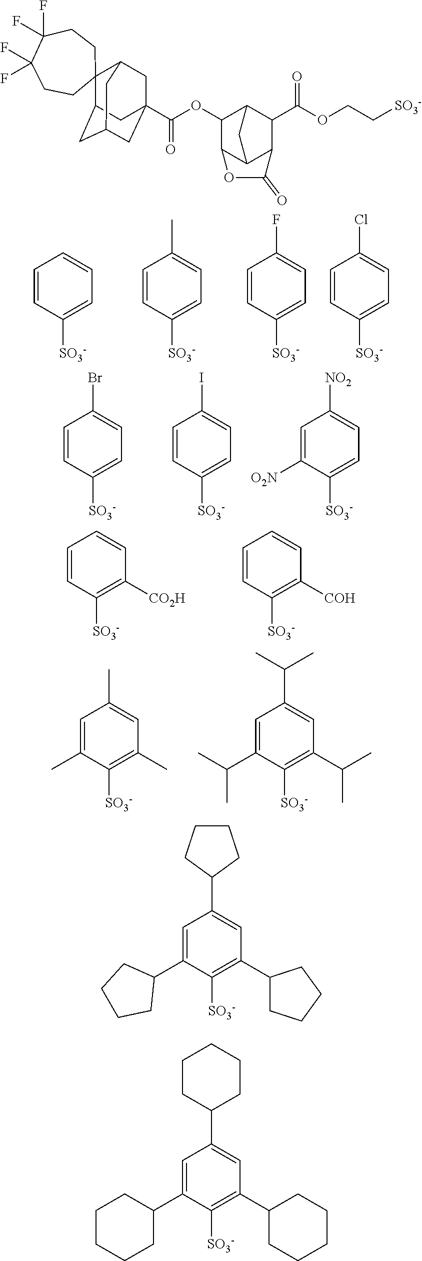

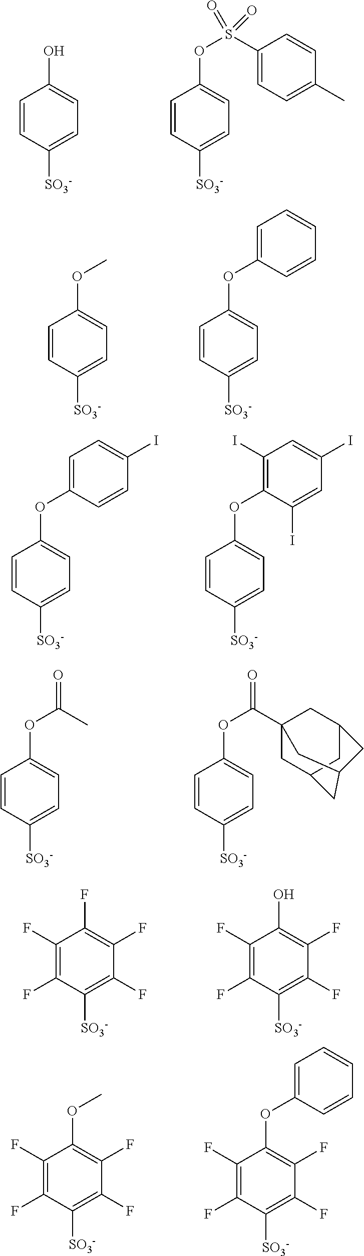

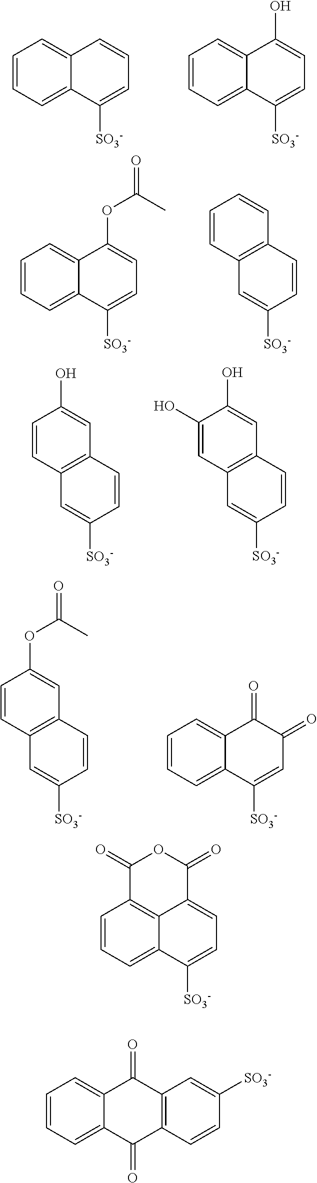

Specific examples of an anion of the onium salt having the formula (2) include, but are not limited to, those listed below.

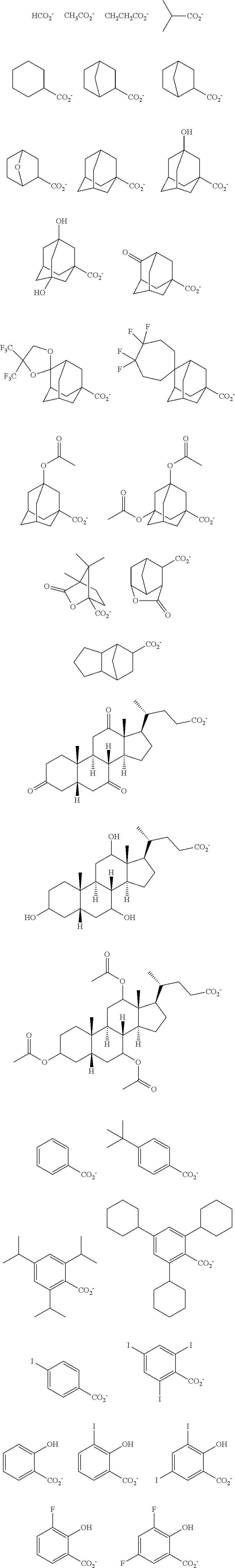

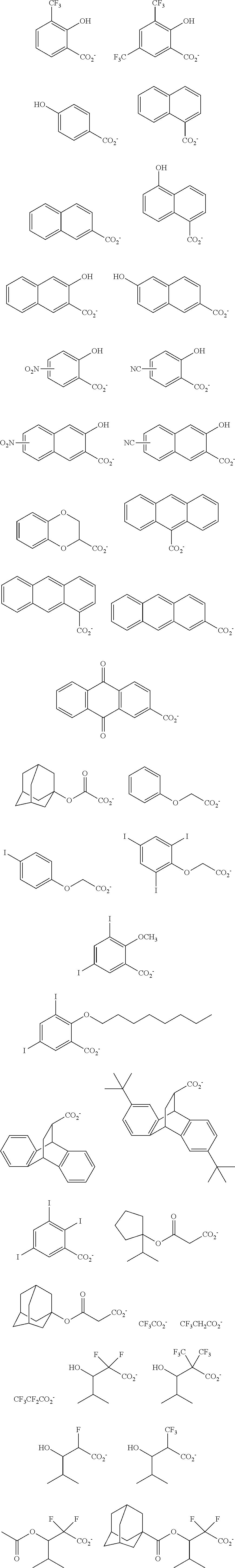

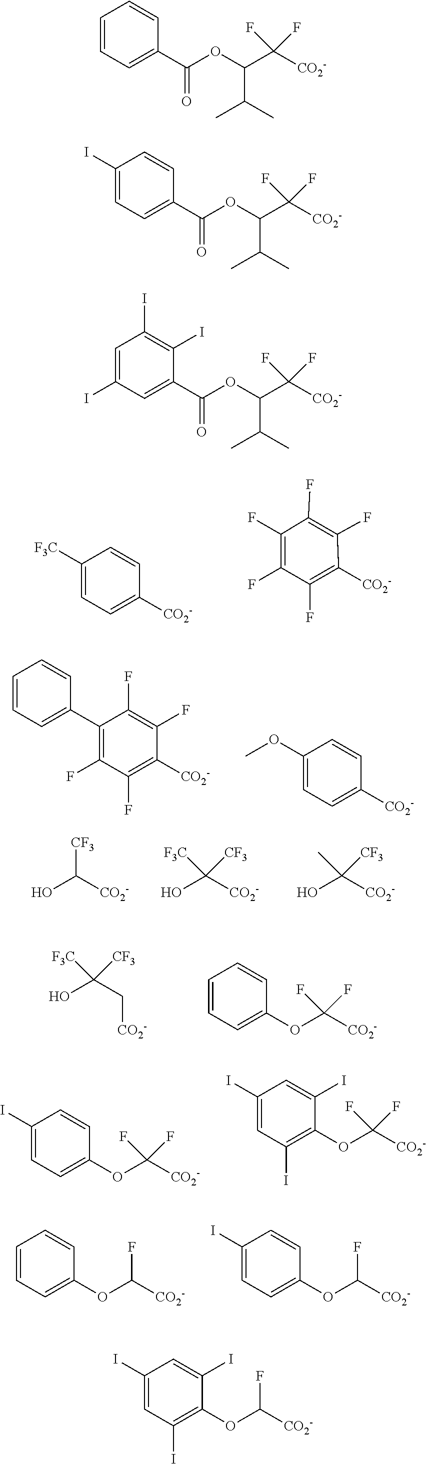

In the formulas (1) and (2), Mq⁺ is an onium cation. Preferable examples of the onium cation include a sulfonium cation, an iodonium cation, and an ammonium cation. Specific examples of the sulfonium cation include, but are not limited to, those similar to those exemplified as specific examples of the sulfonium salt monomer having the formula (a), those described in paragraphs [0102] to [0125] of JP-A 2024-3744, those described in paragraphs [0044] to [0049] of WO 2024/128017, those described in paragraphs [0035] to [0046] of JP 7491173, and those having the formula (d3).

Specific examples of the iodonium cation include, but are not limited to, those described in paragraph [0181] of JP-A 2024-000259.

Specific examples of the ammonium cation include those having the following formula (am-1).

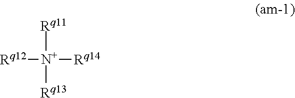

In the formula (am-1), R^{q11} to R^{q14} are each independently a C₁-C₄₀ hydrocarbyl group which may contain a heteroatom. R^{q11} and R^{q12} may be bonded to each other to form a ring together with a nitrogen atom to which they are bonded. Specific examples of the hydrocarbyl group include those similar to those exemplified as the hydrocarbyl group of R¹ to R³ in the description of the formula (a).

Specific examples of the ammonium cation having the formula (am-1) include, but are not limited to, those listed below.

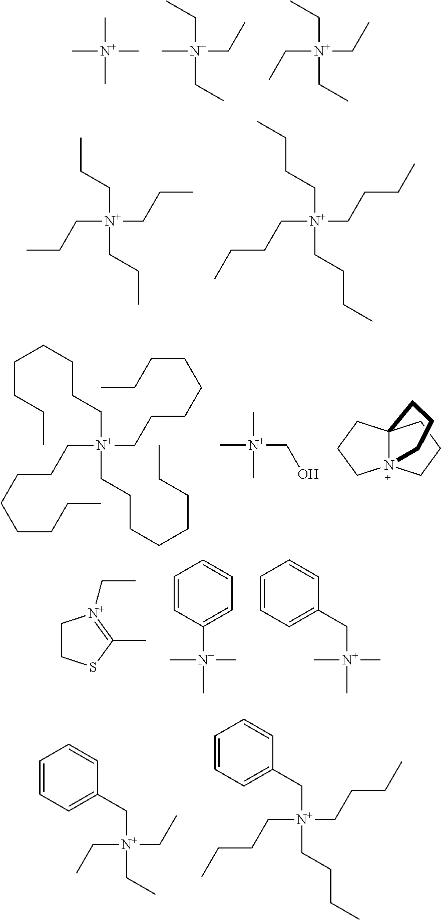

Specific examples of the onium salt having the formula (1) or (2) include any combination of the anion and the cation described above. Note that these onium salts are easily prepared by an ion exchange reaction using a known organic chemical method. For the ion exchange reaction, for example, JP-A 2007-145797 can be referred to.

The onium salt having the formula (1) or (2) acts as a quencher in the chemically amplified resist composition of the present invention. This is because each counter anion of the onium salt is a conjugate base of a weak acid. The weak acid as used herein means an acid having an acidity at which an acid-unstable group of an acid-unstable group-containing unit used for the base polymer cannot be deprotected. The onium salt having the formula (1) or (2) functions as a quencher when used in combination with an onium salt photoacid generator having a conjugate base of a strong acid such as a sulfonic acid whose α-position is fluorinated as a counter anion. That is, in a case where an onium salt that generates a strong acid such as a sulfonic acid in which an α-position is fluorinated and an onium salt salt that generates a weak acid such as a sulfonic acid that is not fluorinated or a carboxylic acid are mixed and used, when a strong acid generated from a photoacid generator by irradiation with a high energy ray collides with an unreacted onium salt having a weak acid anion, the weak acid is released by salt exchange to generate an onium salt salt having a strong acid anion. In this process, the strong acid is exchanged with a weak acid having lower catalytic ability, and therefore the acid is apparently deactivated and acid diffusion can be controlled.

As the quencher (C), an onium salt having a sulfonium cation and a phenoxide anion moiety in the same molecule described in JP 6848776, an onium salt having a sulfonium cation and a carboxylate anion moiety in the same molecule described in JP 6583136 and JP-A 2020-200311, and an onium salt having an iodonium cation and a carboxylate anion moiety in the same molecule described in JP 6274755 can also be used.

Here, when the photoacid generator that generates a strong acid is an onium salt, as described above, a strong acid generated by irradiation with a high energy ray can be exchanged with a weak acid, but on the other hand, it is considered that it is difficult for a weak acid generated by irradiation with a high energy ray to perform salt exchange by colliding with an unreacted onium salt that generates a strong acid. This is due to a phenomenon that the onium cation easily forms an ion pair with an anion of a stronger acid.

When the chemically amplified resist composition of the present invention contains the onium salt having the formula (1) or (2) as the quencher (C), the content thereof is preferably 0.1 to 20 parts by weight, and more preferably 0.1 to 10 parts by weight per 80 parts by weight of the base polymer (A). If the content of the onium salt quencher is in the above range, resolution is good, and sensitivity is not significantly lowered, which is preferable. The onium salt having the formula (1) or (2) may be used singly or in combination of two or more types thereof.

The chemically amplified resist composition of the present invention may contain a nitrogen-containing compound as the quencher (C). Specific examples of the nitrogen-containing compound include primary, secondary, and tertiary amine compounds described in paragraphs [0146] to [0164] of JP-A 2008-111103, particularly an amine compound having a hydroxy group, an ether bond, an ester bond, a lactone ring, a cyano group, or a sulfonate bond. Examples thereof further include a compound obtained by protecting a primary or secondary amine with a carbamate group, described in JP 3790649.

In addition, a sulfonic acid sulfonium salt having a nitrogen-containing substituent may be used as the nitrogen-containing compound. Such a compound functions as a quencher in an unexposed portion, and functions as a so-called photodegradable base that loses quencher ability by neutralization with an acid generated by the compound itself in an exposed portion. By using the photodegradable base, a contrast between the exposed portion and the unexposed portion can be further enhanced. As the photodegradable base, for example, JP-A 2009-109595 and JP-A 2012-46501 can be referred to.

When the chemically amplified resist composition of the present invention contains a nitrogen-containing compound as the quencher (C), the content thereof is preferably 0.001 to 12 parts by weight, and more preferably 0.01 to 8 parts by weight per 80 parts by weight of the base polymer (A). The nitrogen-containing compound may be used singly or in combination of two or more types thereof.

### (D) Acid Generator

The chemically amplified resist composition of the present invention may contain an acid generator as long as the effect of the present invention is not impaired. Examples of the acid generator include a compound (photoacid generator) that generates an acid in response to an actinic ray or a radiation. The photoacid generator is not particularly limited as long as it is a compound that generates an acid by irradiation with a high energy ray, but a photoacid generator that generates a sulfonic acid, an imide acid, or a methide acid is preferable. Suitable examples of the photoacid generator include a sulfonium salt, an iodonium salt, sulfonyldiazomethane, N-sulfonyloxyimide, and an oxime-O-sulfonate acid generator. Specific examples of the acid generator include those described in paragraphs to of JP-A 2008-111103.

Examples of a suitable photoacid generator include those having the following formula (3) or (4).

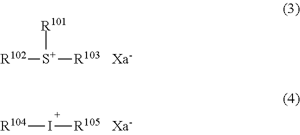

In the formula (3), R¹⁰¹ to R¹⁰⁵ are each independently a halogen atom or a C₁-C₂₀ hydrocarbyl group which may contain a heteroatom. Any two of R¹⁰¹, R¹⁰², and R¹⁰³ may be bonded to each other to form a ring together with a sulfur atom to which they are bonded. Specific examples of the hydrocarbyl group include those similar to those exemplified as the hydrocarbyl group of R¹ to R³ in the description of the formula (a).

Specific examples of the cation of the sulfonium salt having the formula (3) include, but are not limited to, those exemplified as specific examples of the cation of the sulfonium salt monomer having the formula (a), those described in paragraphs [0102] to [0125] of JP-A 2024-3744, those described in paragraphs [0044] to [0049] of WO 2024/128017, those described in paragraphs [0035] to [0046] of JP 7491173, and those exemplified as specific examples of the sulfonium cation having the formula (d3). Specific examples of the cation of the iodonium salt having the formula (4) include, but are not limited to, those described in paragraph [0181] of JP-A 2024-259.

In the formulas (3) and (4), Xa⁻ is an anion of a strong acid. Examples of the anion of a strong acid include those having any of the following formulas (Xa-1) to (Xa-4).

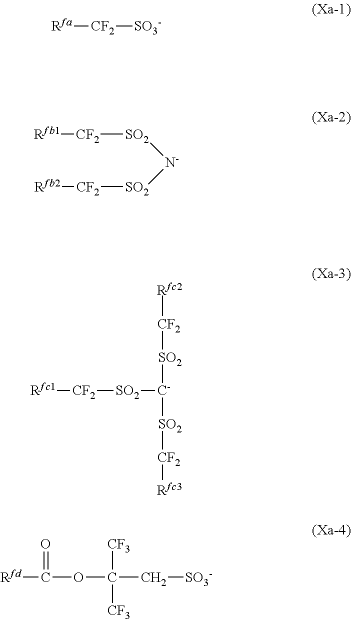

In the formula (Xa-1), R^{fa} is a fluorine atom or a C₁-C₆₀ hydrocarbyl group which may contain a heteroatom. The hydrocarbyl group may be saturated or unsaturated, and may be linear, branched, or cyclic. Specific examples thereof include those similar to those exemplified as the hydrocarbyl group of R^{fa1} in the formula (Xa-1-1) described below.

The anion having the formula (Xa-1) preferably has the following formula (Xa-1-1):

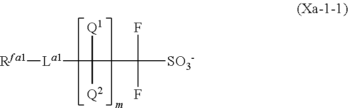

In the formula (Xa-1-1), Q¹ and Q² are each independently a hydrogen atom, a fluorine atom, or a C₁-C₆ fluorinated saturated hydrocarbyl group, but at least one of Q¹ and Q² is preferably a trifluoromethyl group for improving solvent solubility. m is 0, 1, 2, 3, or 4, and particularly preferably 1. R^{fa1} is a C₁-C₄₀ hydrocarbyl group which may contain a heteroatom. The heteroatom is preferably an oxygen atom, a nitrogen atom, a sulfur atom, a halogen atom, or the like, and more preferably an oxygen atom. The hydrocarbyl group particularly preferably has 6 to 30 carbon atoms from a viewpoint of obtaining high resolution in fine pattern formation.

In the formula (Xa-1-1), the C₁-C₄₀ hydrocarbyl group of R^{fa1} may be saturated or unsaturated, and may be linear, branched, or cyclic. Specific examples thereof include: a C₁-C₄₀ alkyl group such as a methyl group, an ethyl group, a n-propyl group, an isopropyl group, a n-butyl group, an isobutyl group, a sec-butyl group, a tert-butyl group, a pentyl group, a neopentyl group, a hexyl group, a heptyl group, a 2-ethylhexyl group, a nonyl group, an undecyl group, a tridecyl group, a pentadecyl group, a heptadecyl group, or an icosyl group; a C₃-C₄₀ cyclic saturated hydrocarbyl group such as a cyclopentyl group, a cyclohexyl group, a 1-adamantyl group, a 2-adamantyl group, a 1-adamantylmethyl group, a norbornyl group, a norbornylmethyl group, a tricyclodecyl group, a tetracyclododecyl group, a tetracyclododecylmethyl group, or a dicyclohexylmethyl group; a C₂-C₄₀ unsaturated aliphatic hydrocarbyl group such as a 2-propenyl group or a 3-cyclohexenyl group; a C₆-C₄₀ aryl group such as a phenyl group, a 1-naphthyl group, a 2-naphthyl group, or a 9-fluorenyl group; a C₇-C₄₀ aralkyl group such as a benzyl group or a diphenylmethyl group; a C₇-C₄₀ aromatic ring-containing polycyclic hydrocarbyl group such as a 9,10-ethano-9,10-dihydroanthryl group or a 6,13-ethano-6,13-dihydropentacenyl group; a C₁₇-C₄₀ hydrocarbyl group having a steroid skeleton; and a group obtained by combining these groups.

Some or all of hydrogen atoms of the hydrocarbyl group may be replaced with a group containing a heteroatom such as an oxygen atom, a sulfur atom, a nitrogen atom, or a halogen atom, some of (—CH₂—)s in the hydrocarbyl group may be replaced with a group containing a heteroatom such as an oxygen atom, a sulfur atom, or a nitrogen atom, and as a result, the hydrocarbyl group may contain a hydroxy group, a fluorine atom, a chlorine atom, a bromine atom, an iodine atom, a cyano group, a nitro group, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like. Specific examples of the hydrocarbyl group containing a heteroatom include a tetrahydrofuryl group, a methoxymethyl group, an ethoxymethyl group, a methylthiomethyl group, an acetamidomethyl group, a trifluoroethyl group, a (2-methoxyethoxy) methyl group, an acetoxymethyl group, a 2-carboxy-1-cyclohexyl group, a 2-oxopropyl group, a 4-oxo-1-adamantyl group, and a 3-oxocyclohexyl group.

In the formula (Xa-1-1), Lal is a single bond, an ether bond, an ester bond, a sulfonate bond, a carbonate bond, or a carbamate bond, but is preferably an ether bond or an ester bond, and more preferably an ester bond from a viewpoint of synthesis.

Specific examples of the anion having the formula (Xa-1) include, but are not limited to, those listed below. Note that, in the following formulas, Q¹ is as defined above, and Ac is an acetyl group.

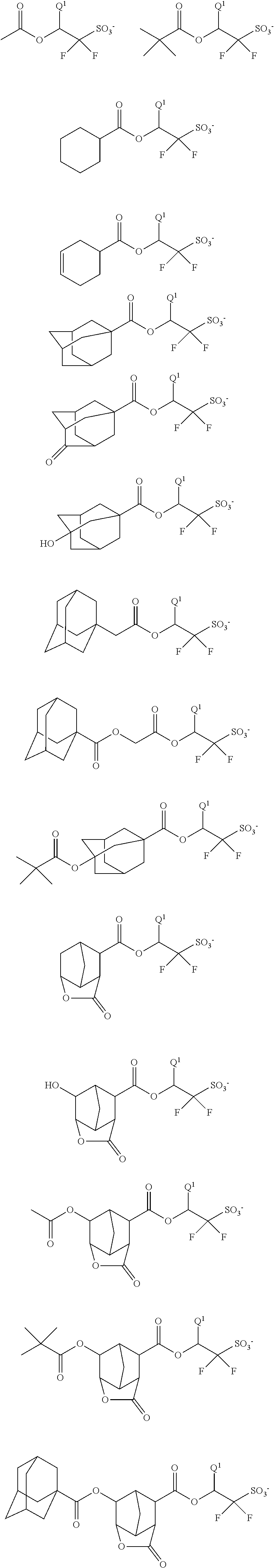

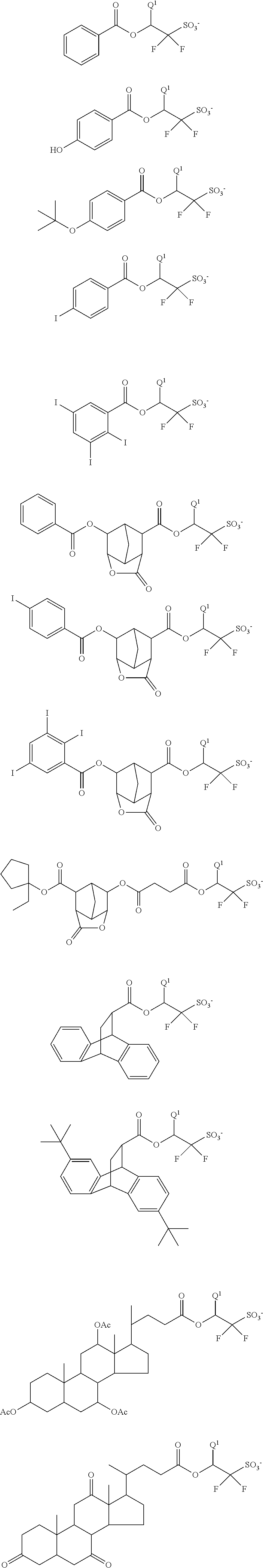

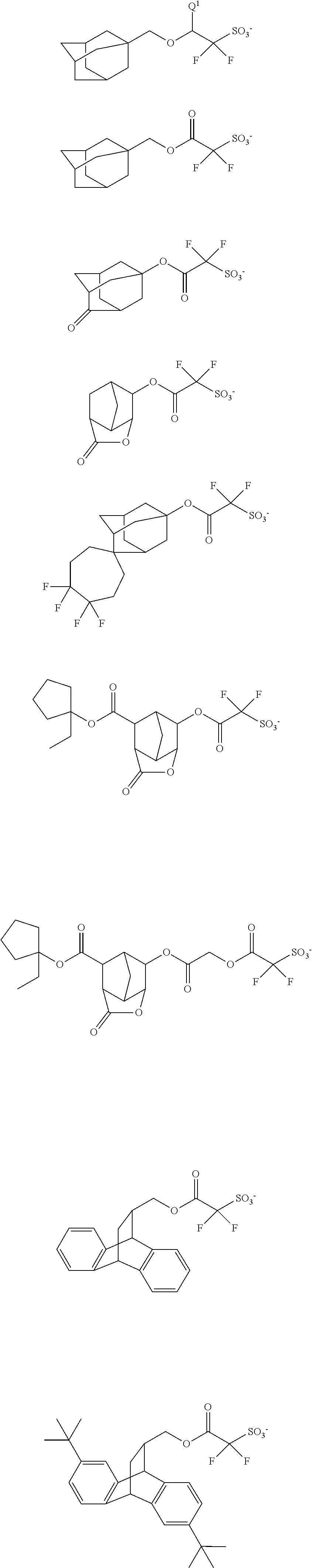

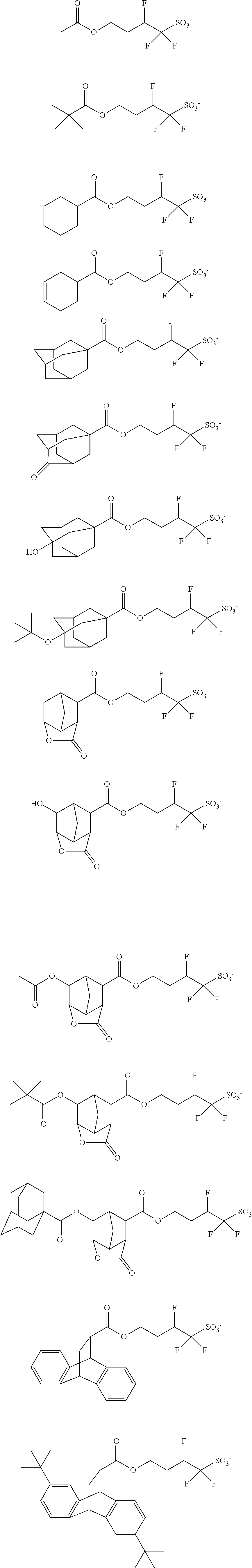

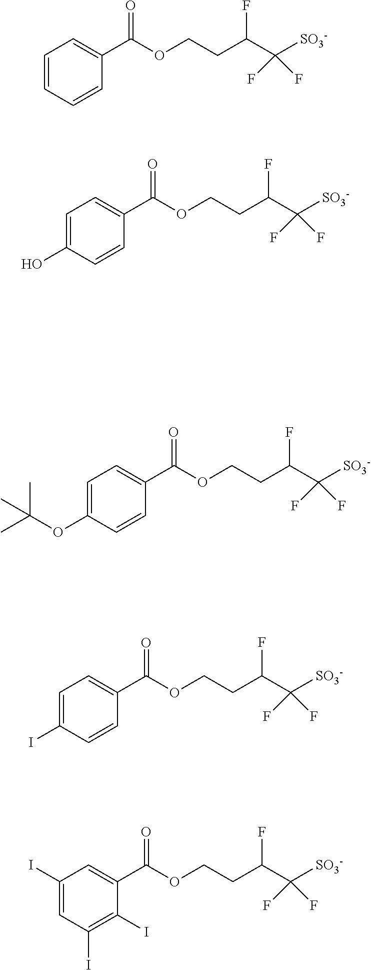

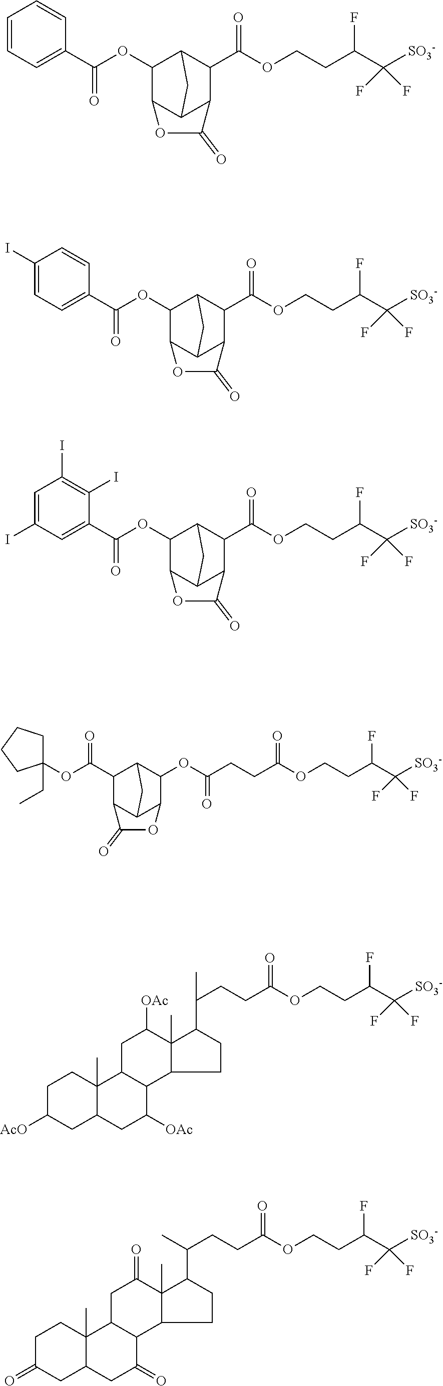

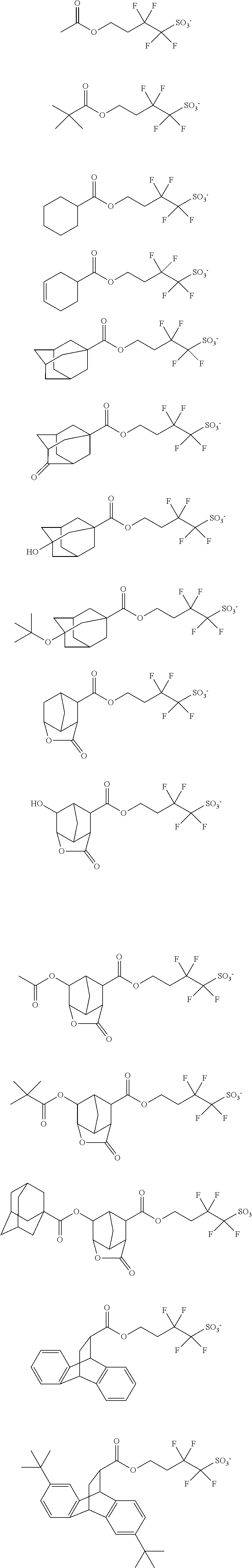

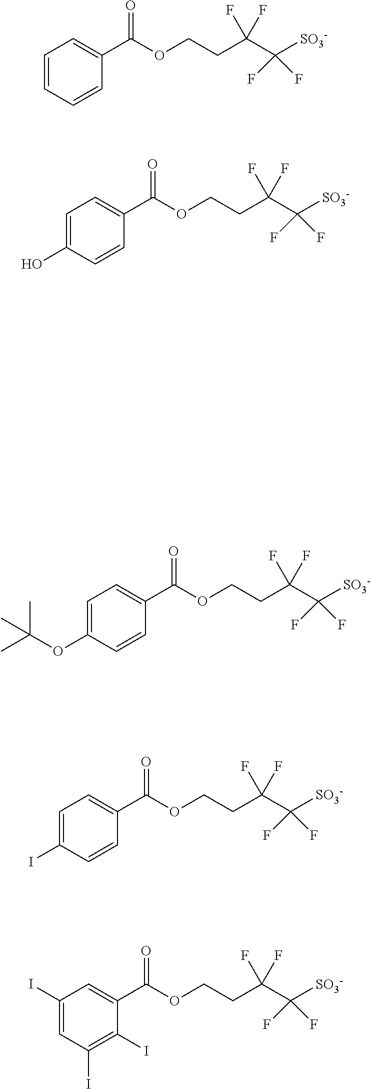

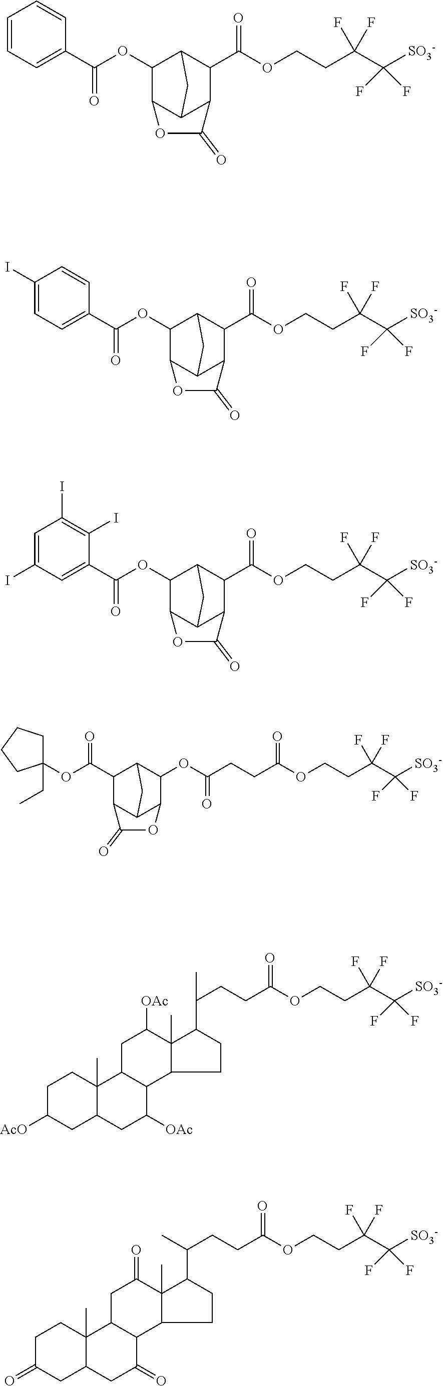

In the formula (Xa-2), R^{fb1} and R^{fb2} are each independently a fluorine atom or a C₁-C₄₀ hydrocarbyl group which may contain a heteroatom. The hydrocarbyl group may be saturated or unsaturated, and may be linear, branched, or cyclic. Specific examples thereof include those similar to those exemplified as the hydrocarbyl group of R^{fa1} in the formula (Xa-1-1). R^{fb1} and R^{fb2} are each preferably a fluorine atom or a linear C₁-C₄ fluorinated alkyl group. R^{fb1} and R^{fb2} may be bonded to each other to form a ring together with a group to which they are bonded (—CF₂—SO₂—N⁻—SO₂—CF₂—), and in this case, a group obtained by bonding of R^{fb1} and R^{fb2} is preferably a fluorinated ethylene group or a fluorinated propylene group.

In the formula (Xa-3), R^{fc1}, R^{fc2}, and R^{fc3} are each independently a fluorine atom or a C₁-C₄₀ hydrocarbyl group which may contain a heteroatom. The hydrocarbyl group may be saturated or unsaturated, and may be linear, branched, or cyclic. Specific examples thereof include those similar to those exemplified as the hydrocarbyl group of R^{fa1} in the formula (Xa-1-1). R^{fc1}, R^{fc2}, and R_{fe3} are each preferably a fluorine atom or a linear C₁-C₄ fluorinated alkyl group. R^{fc1} and R^{fc2} may be bonded to each other to form a ring together with a group to which they are bonded (—CF₂—SO₂—C⁻—SO₂—CF₂—), and in this case, a group obtained by bonding of R^{fc1} and R^{fc2} is preferably a fluorinated ethylene group or a fluorinated propylene group.

In the formula (Xa-4), R^{fd} is a C₁-C₄₀ hydrocarbyl group which may contain a heteroatom. The hydrocarbyl group may be saturated or unsaturated, and may be linear, branched, or cyclic. Specific examples thereof include those similar to those exemplified as the hydrocarbyl group of R^{fa1} in the formula (Xa-1-1). Some or all of hydrogen atoms of the hydrocarbyl group may be replaced with a group containing a heteroatom such as an oxygen atom, a sulfur atom, a nitrogen atom, or a halogen atom, some of (—CH₂—)s in the hydrocarbyl group may be replaced with a group containing a heteroatom such as an oxygen atom, a sulfur atom, or a nitrogen atom, and as a result, the hydrocarbyl group may contain a hydroxy group, a fluorine atom, a chlorine atom, a bromine atom, an iodine atom, a cyano group, a nitro group, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like.

Specific examples of the anion having the formula (Xa-4) include, but are not limited to, those listed below.

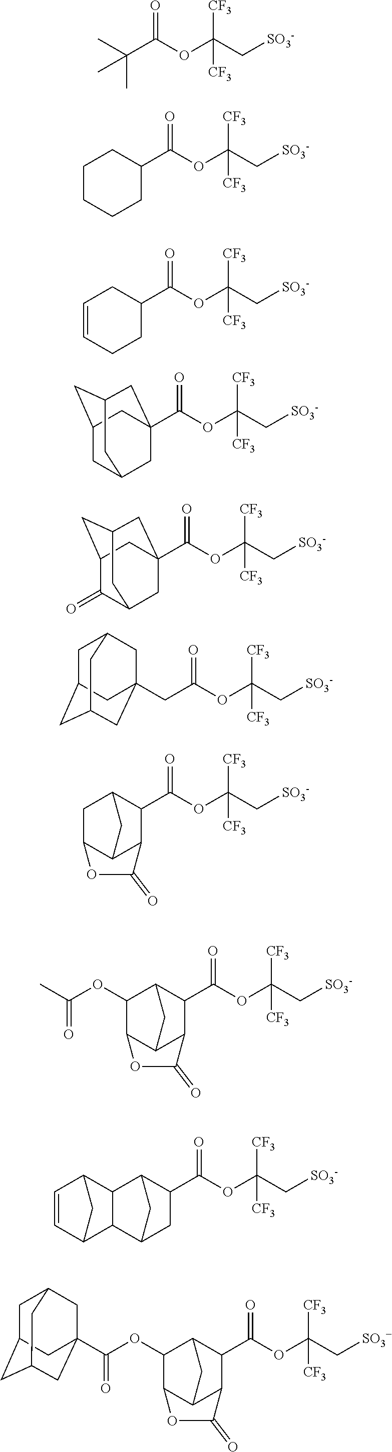

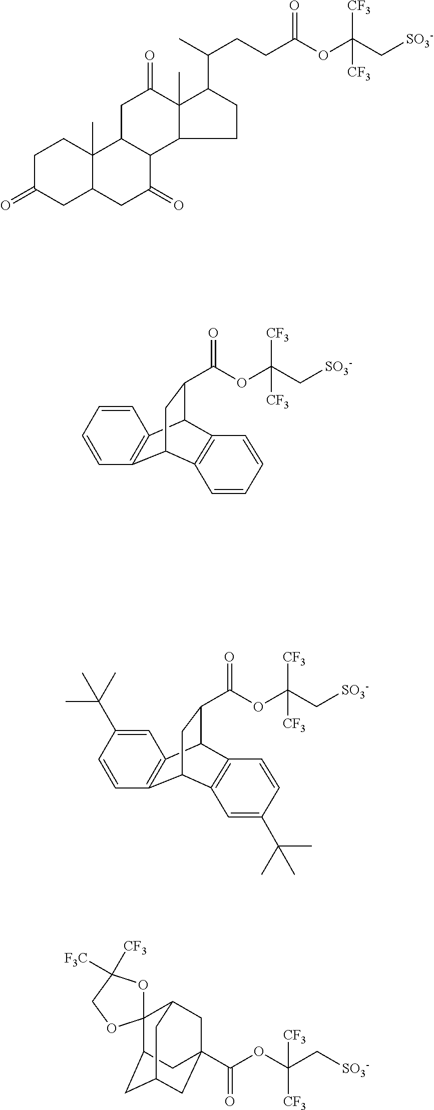

Examples of the non-nucleophilic counter ion further include an anion having an aromatic ring with an iodine atom or a bromine atom as a substituent. Specific examples of such an anion include those having the following formula (Xa-5).

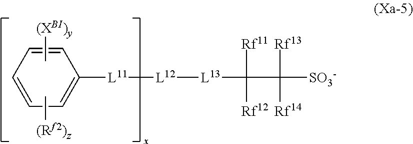

In the formula (Xa-5), x is 1, 2, or 3. y is 1, 2, 3, 4, or 5.
z is 0, 1, 2, or 3. Provided that 1≤y+z≤5. y is preferably 1, 2, or 3, and more preferably 2 or 3. z is preferably 0, 1, or 2.

In the formula (Xa-5), X^{B1} is an iodine atom or a bromine atom. When x and/or y is 2 or more, X^{B1}s may be the same as or different from each other.

In the formula (Xa-5), L¹¹ is a single bond, an ether bond, an ester bond, a sulfonate bond, a carbonate bond, a carbamate bond, or a C₁-C₆ saturated hydrocarbylene group, and some of (—CH₂—)s of the hydrocarbylene group may be replaced with an ether bond or an ester bond. The saturated hydrocarbylene group may be linear, branched, or cyclic.

In the formula (Xa-5), L¹² is a single bond or a C₁-C₂₀ hydrocarbylene group which may contain a heteroatom when x is 1, and is a C₁-C₂₀ (x+1)-valent hydrocarbon group which may contain a heteroatom when x is 2 or 3.

The C₁-C₂₀ hydrocarbylene group of L¹² may be saturated or unsaturated, and may be linear, branched, or cyclic. Specific examples thereof include: a C₁-C₂₀ alkanediyl group such as a methanediyl group, an ethane-1,1-diyl group, an ethane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, or a dodecane-1,12-diyl group; a C₃-C₂₀ cyclic saturated hydrocarbylene group such as a cyclopentanediyl group, a cyclohexanediyl group, a norbornanediyl group, an adamantanediyl group, or a tricyclo[5.2.1.0^{2,6}]decanediyl group; a C₂-C₂₀ unsaturated aliphatic hydrocarbylene such as vinylene group or a propene-1,3-diyl group; a C₆-C₂₀ arylene group such as a phenylene group, a naphthylene group, or an anthracenediyl group; a C₇-C₂₀ aromatic ring-containing polycyclic hydrocarbylene group such as a 9,10-ethano-9,10-dihydroanthracenediyl group or a 6,13-ethano-6,13-dihydropentacene group; and a group obtained by combining these groups. The C₁-C₂₀ (x+1)-valent hydrocarbon group of L¹² may be saturated or unsaturated, and may be linear, branched, or cyclic. Specific examples thereof include groups obtained by further removing one or two hydrogen atoms from the above-described specific examples of the C₁-C₂₀ hydrocarbylene group.

Some or all of hydrogen atoms of the hydrocarbylene group and the (x+1)-valent hydrocarbon group may be replaced with a group containing a heteroatom such as an oxygen atom, a sulfur atom, a nitrogen atom, or a halogen atom, some of (—CH₂—)s in the hydrocarbylene group and the (x+1)-valent hydrocarbon group may be replaced with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic acid anhydride (—C(—O)—O—C(—O)—), a haloalkyl group, or the like.

In the formula (Xa-5), L¹³ is a single bond, an ether bond, an ester bond, a sulfonate bond, a carbonate bond, or a carbamate bond.

In the formula (Xa-5), R^{fe} is a hydroxy group, a carboxy group, a fluorine atom, a chlorine atom, a bromine atom, an amino group, a C₁-C₂₀ hydrocarbyl group, a C₁-C₂₀ hydrocarbyloxy group, a C₁-C₂₀ hydrocarbylthio group, a C₂-C₂₀ hydrocarbylcarbonyl group, a C₂-C₂₀ hydrocarbyloxycarbonyl group, a C₂-C₂₀ hydrocarbylcarbonyloxy group, a C₁-C₂₀ hydrocarbylsulfonyloxy group, —N(R^{feA}) (R^{feB}), —N(R^{feC})—C(═O)—R^{feD} or—N(R^{feC})—C(═O)—O—R^{feD}, the hydrocarbyl group, the hydrocarbyloxy group, the hydrocarbylthio group, the hydrocarbylcarbonyl group, the hydrocarbyloxycarbonyl group, the hydrocarbylcarbonyloxy group, and the hydrocarbylsulfonyloxy group may contain at least one selected from a fluorine atom, a chlorine atom, a bromine atom, an iodine atom, a hydroxy group, an amino group, an ester bond, and an ether bond. R^{feA} and R^{feB} are each independently a hydrogen atom or a C₁-C₆ saturated hydrocarbyl group. R^{feC} is a hydrogen atom or a C₁-C₆ saturated hydrocarbyl group, and may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. R^{feD} is a C₁-C₁₆ aliphatic hydrocarbyl group, a C₆-C₁₂ aryl group, or a C₇-C₁₅ aralkyl group, and may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. The aliphatic hydrocarbyl group may be saturated or unsaturated, and may be linear, branched, or cyclic. The hydrocarbyl group, hydrocarbyloxy group, hydrocarbylcarbonyl group, hydrocarbyloxycarbonyl group, hydrocarbylcarbonyloxy group, and hydrocarbylsulfonyloxy group may be linear, branched, or cyclic. When x and/or z are 2 or more, R^{fe}s may be the same as or different from each other.

Among these, R^{fe} is preferably a hydroxy group, —N(R^{feC})—C(═O)—R^{feD}, —N(R^{feC})—C(═O)—O—R^{feD}, a fluorine atom, a chlorine atom, a bromine atom, a methyl group, a methoxy group, or the like.

In the formula (Xa-5), Rf¹¹ to Rf¹⁴ are each independently a hydrogen atom, a fluorine atom, or a trifluoromethyl group, and at least one of these is a fluorine atom or a trifluoromethyl group. Rf¹¹ and Rf¹² may be combined to form a carbonyl group. In particular, both Rf¹³ and Rf¹⁴ are preferably fluorine atoms.

Specific examples of the anion having the formula (Xa-5) include, but are not limited to, those listed below. Note that, in the following formulas, X^{B1} is as defined above.

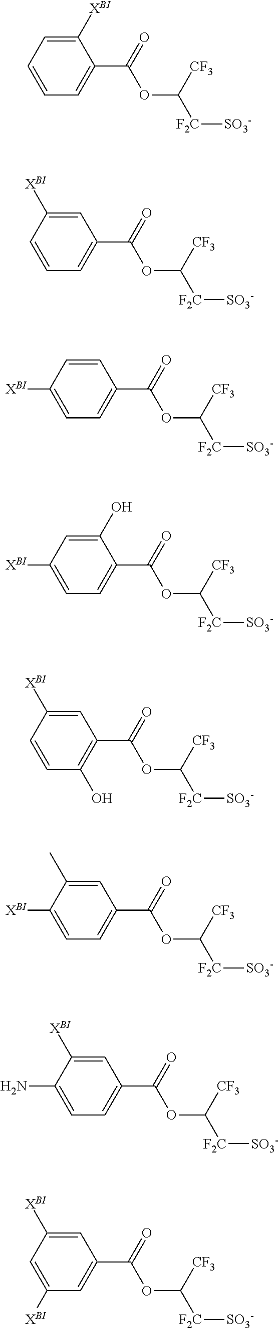

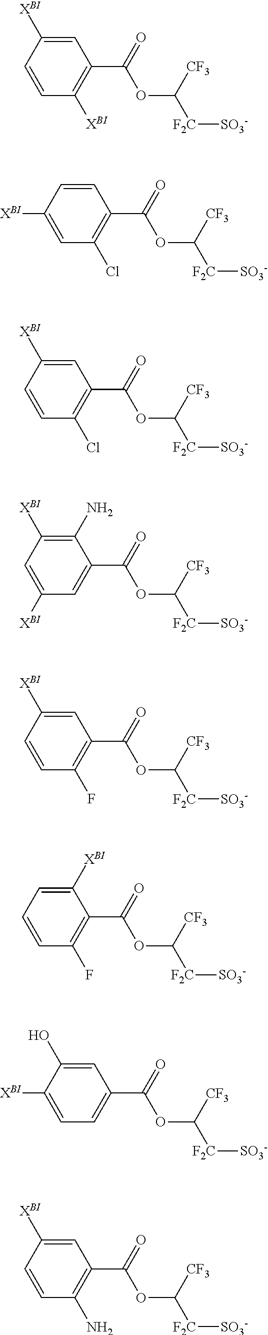

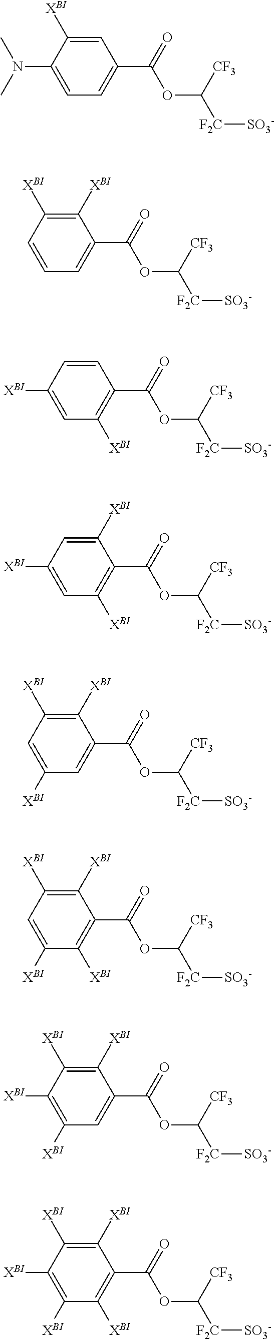

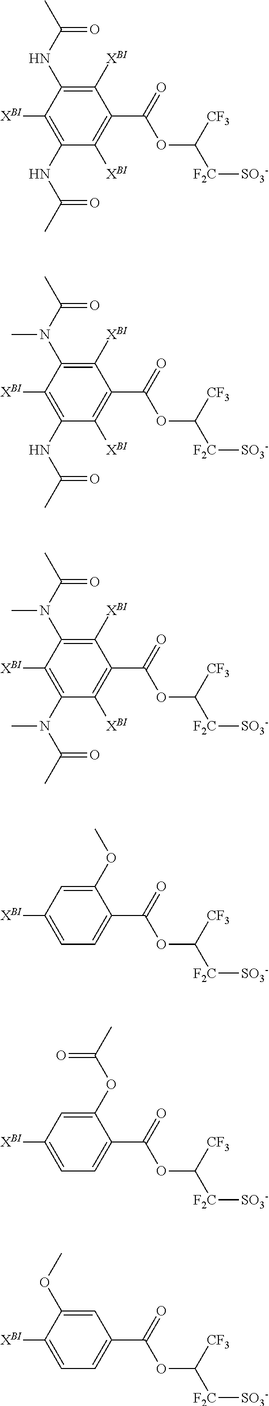

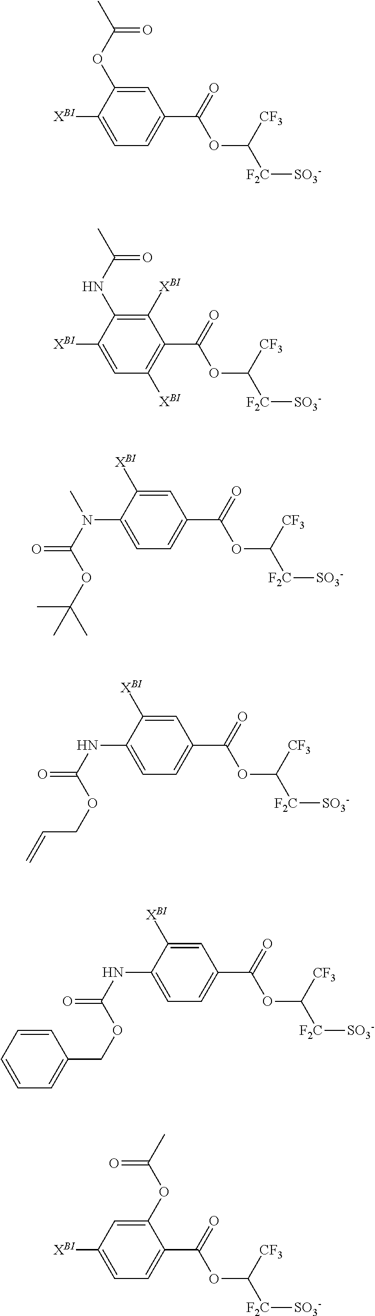

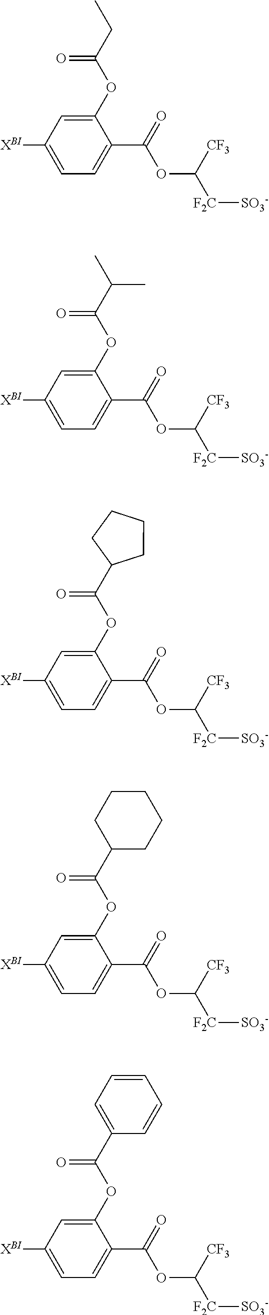

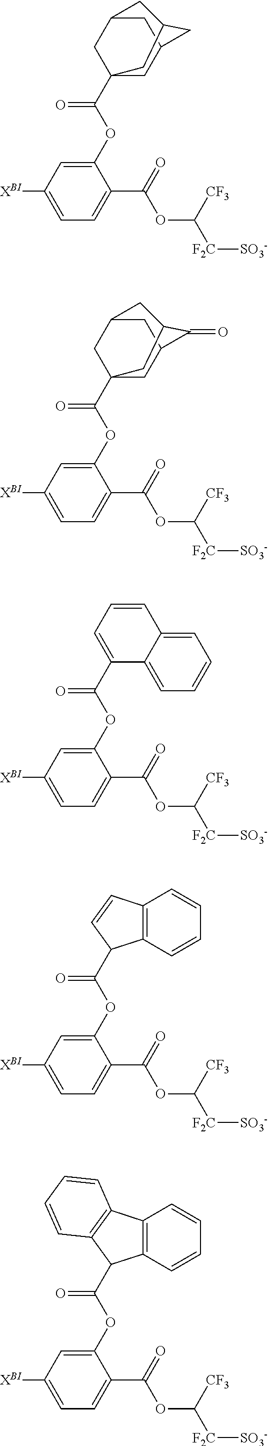

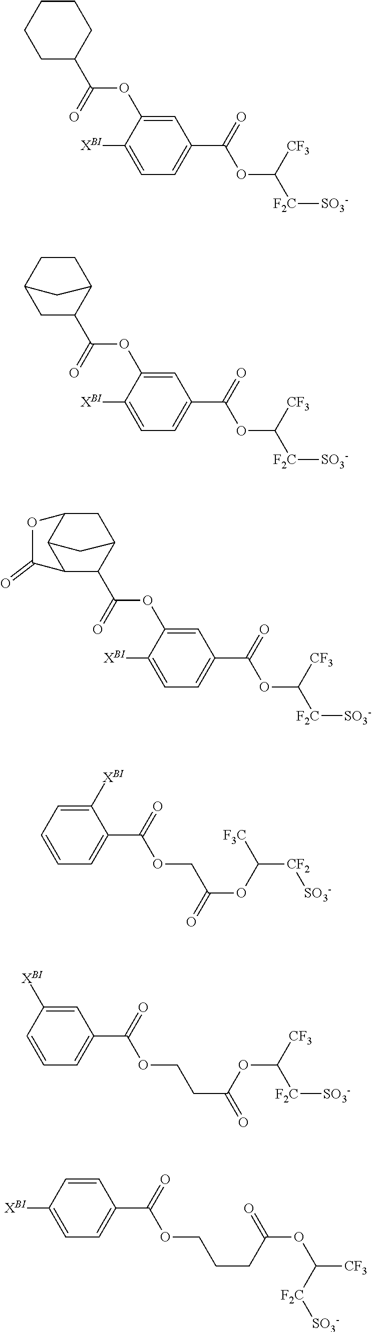

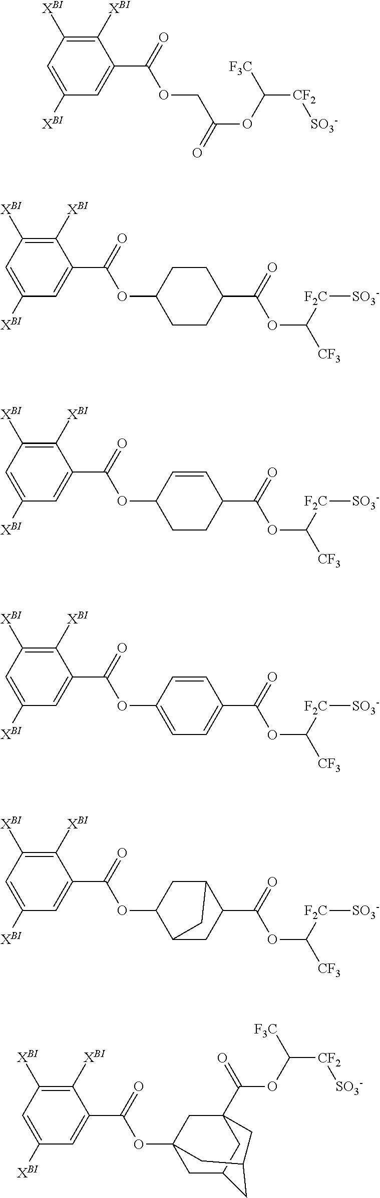

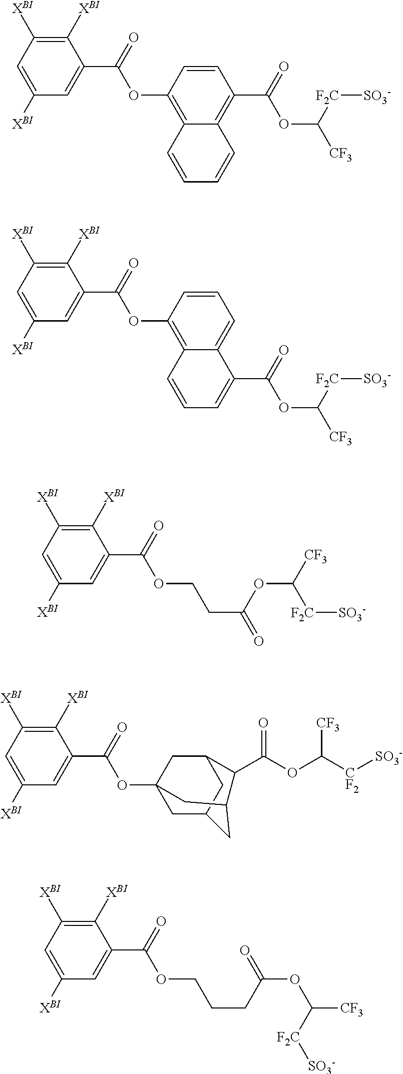

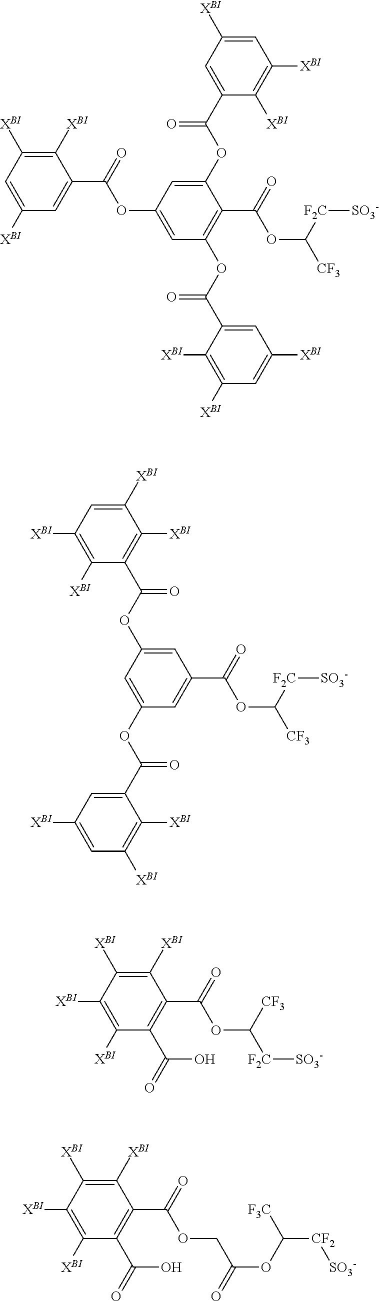

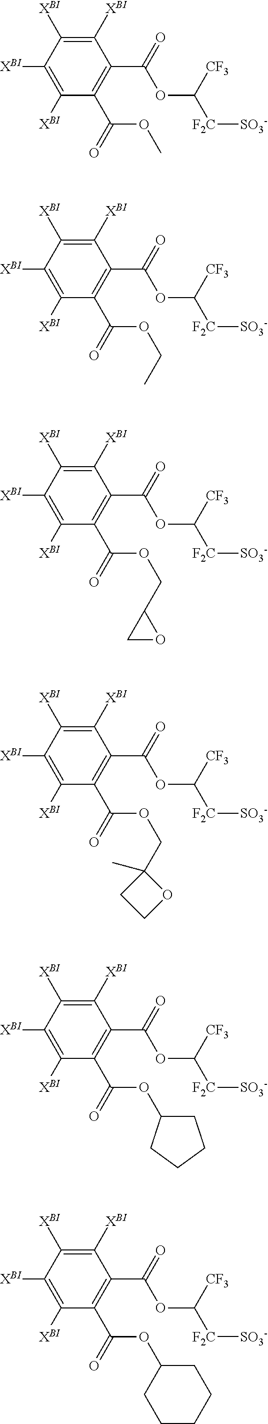

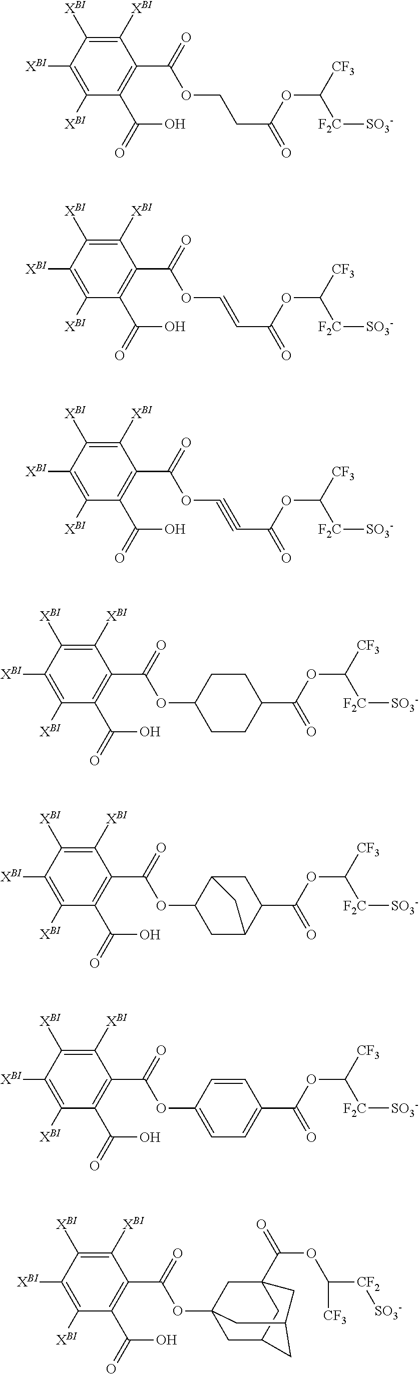

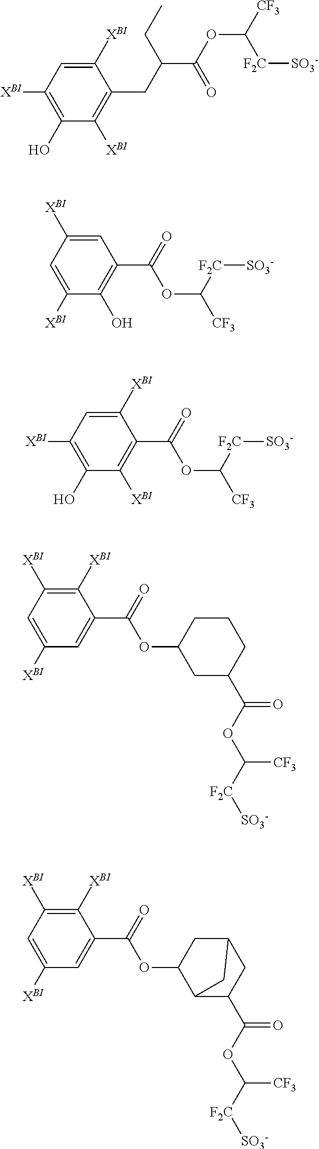

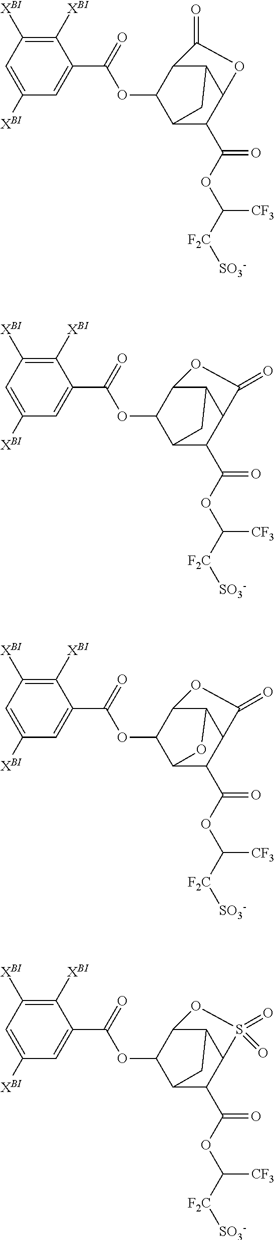

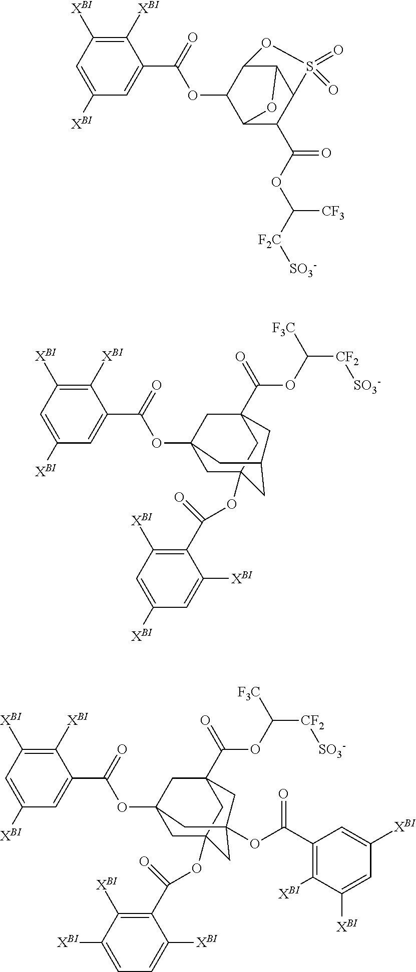

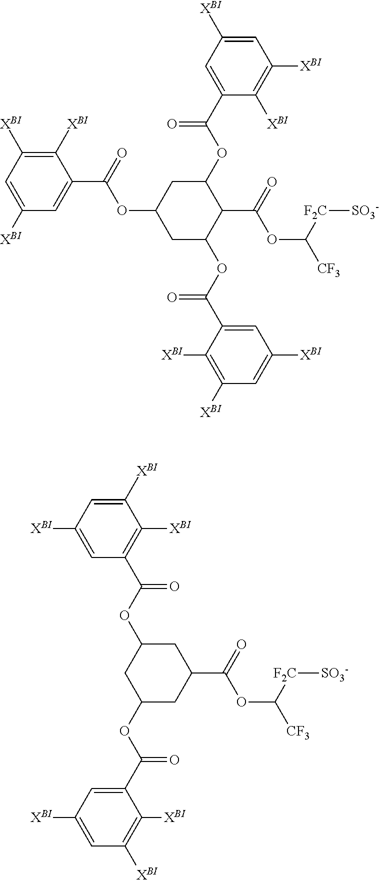

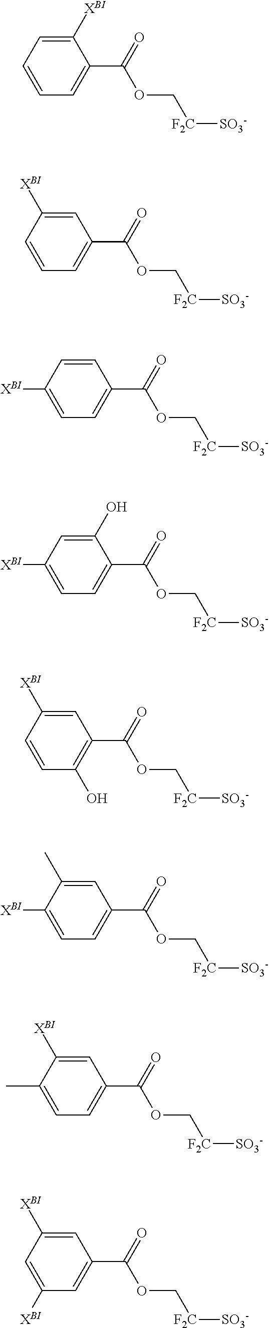

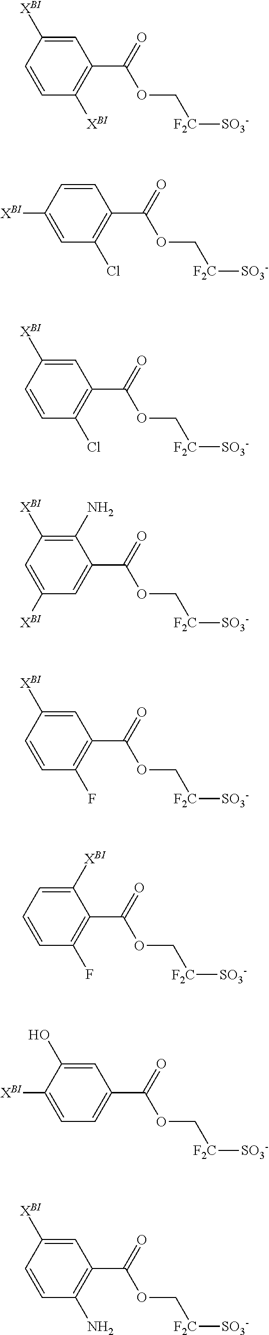

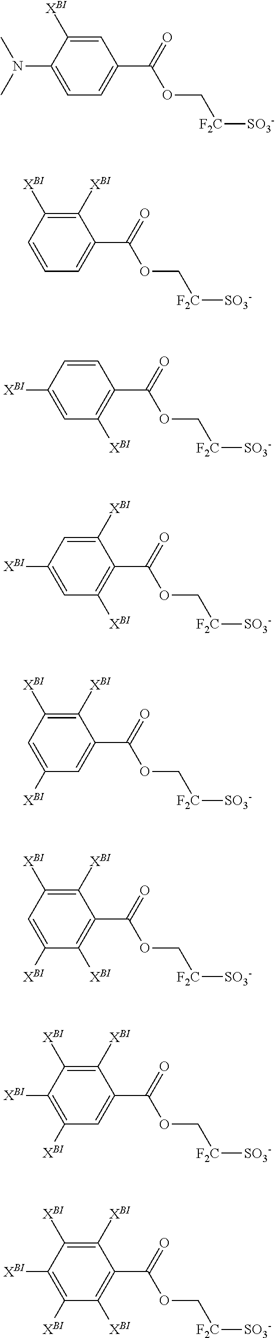

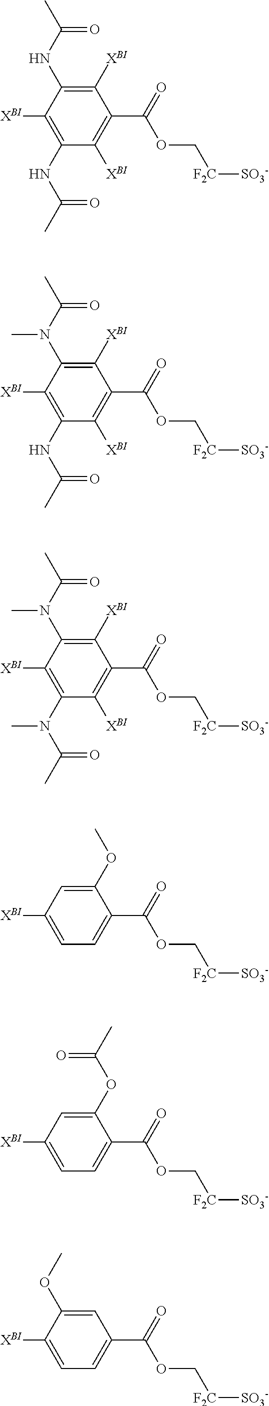

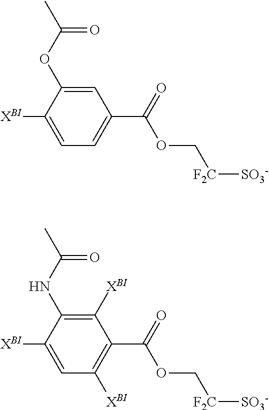

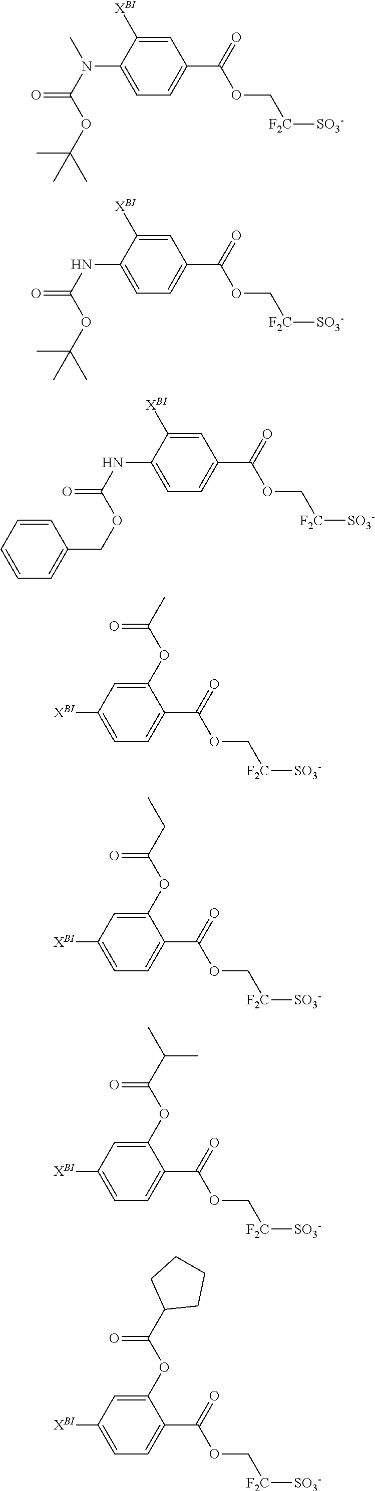

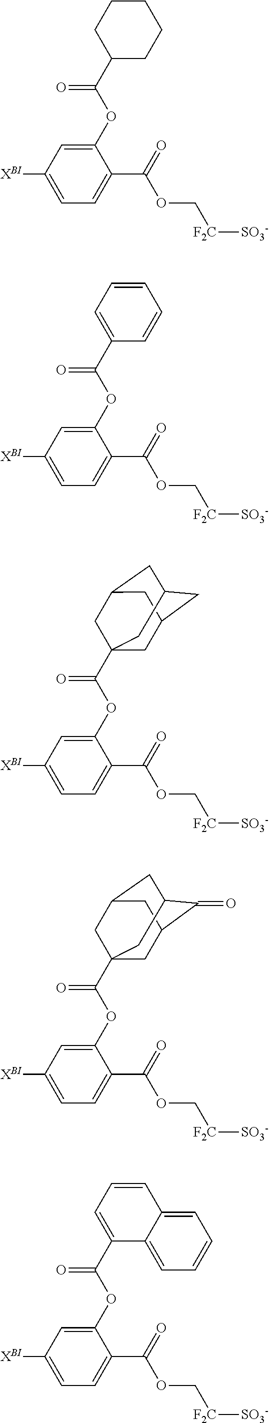

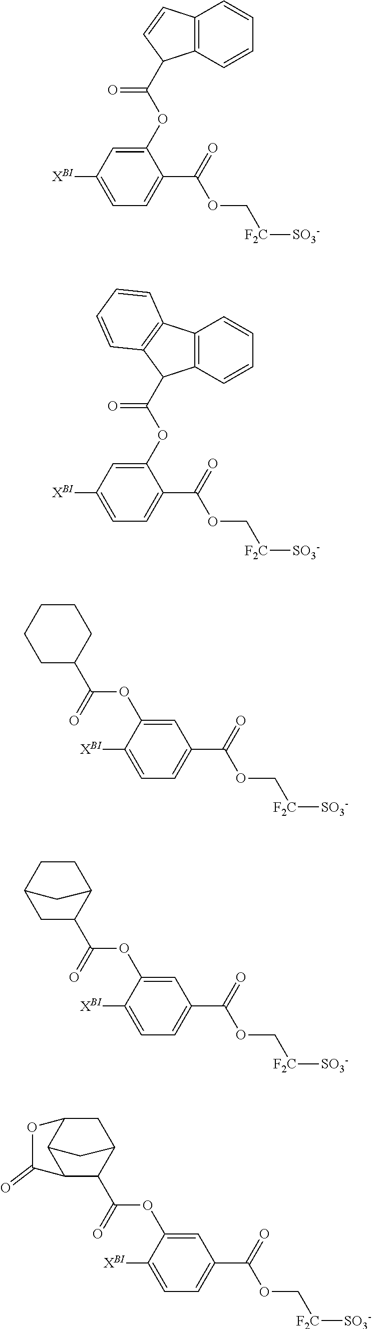

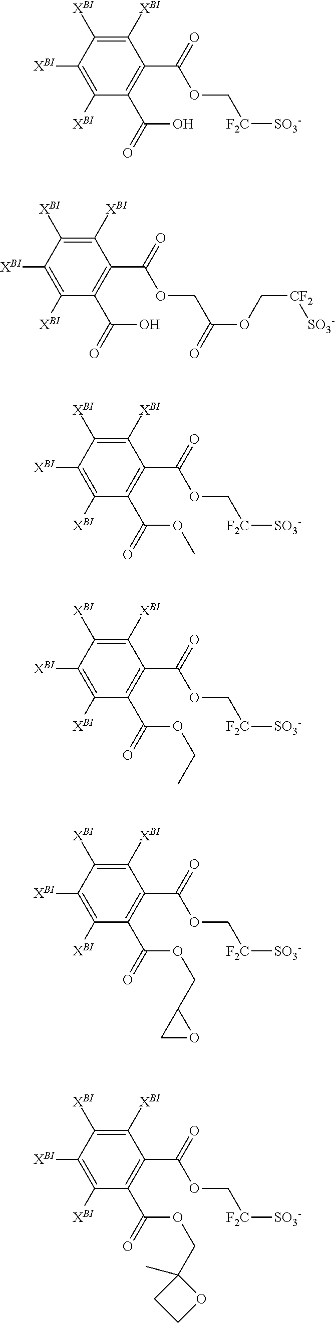

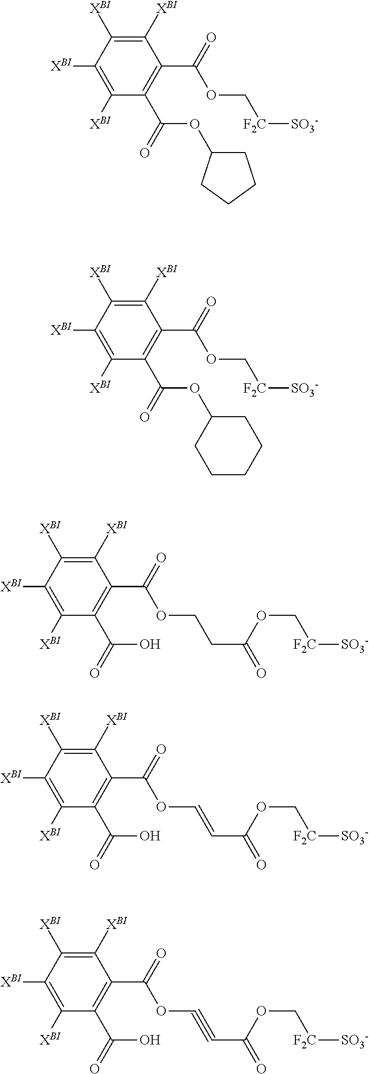

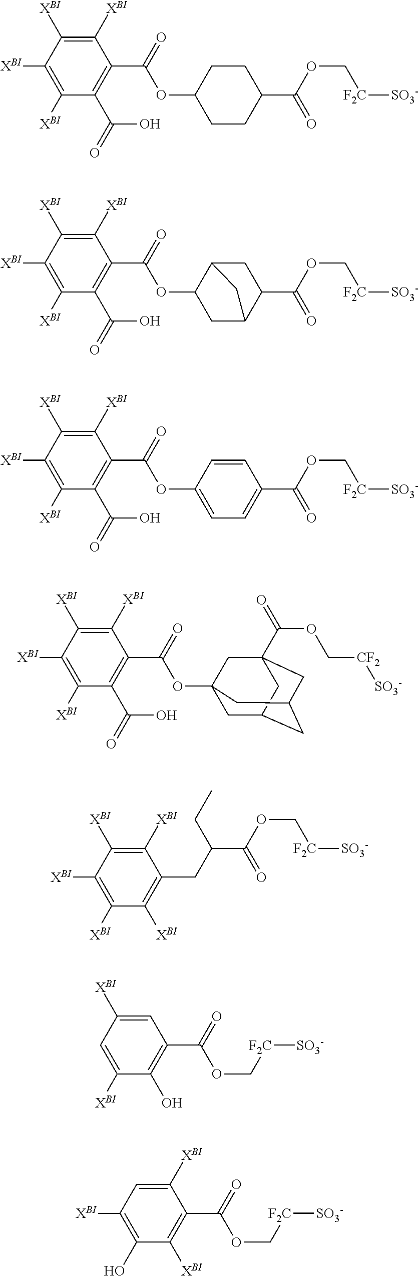

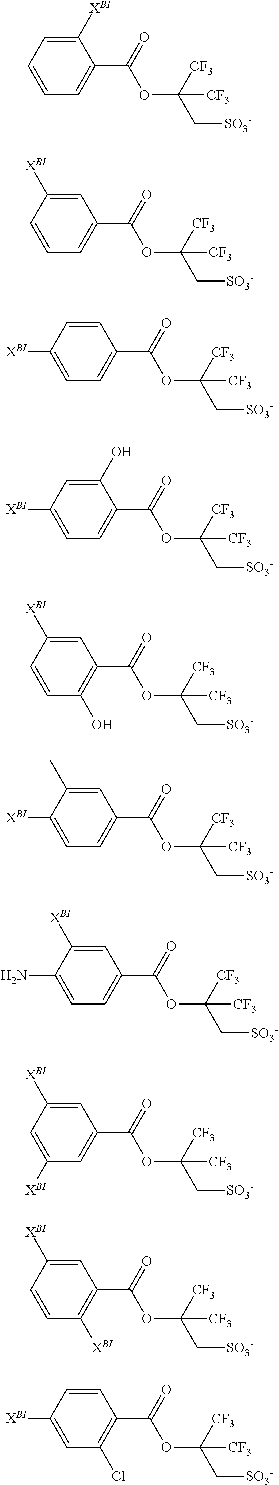

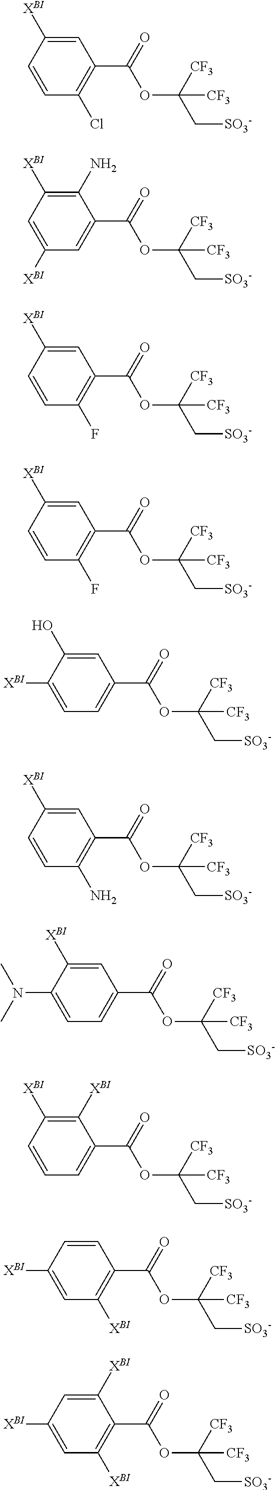

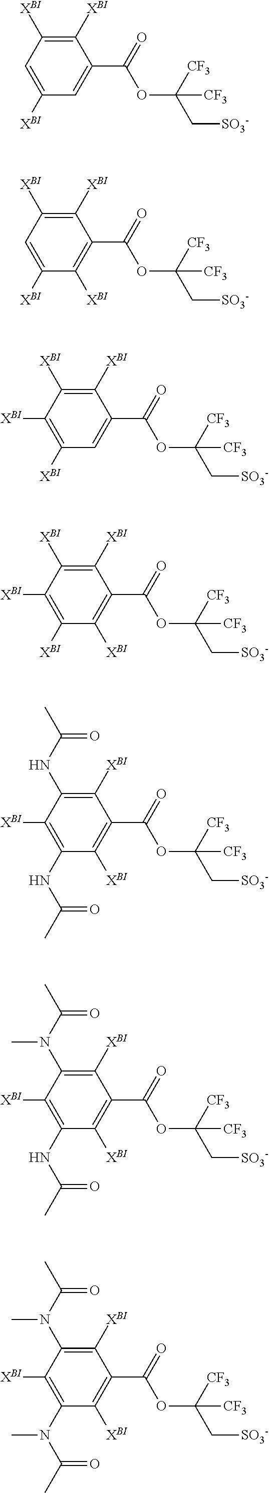

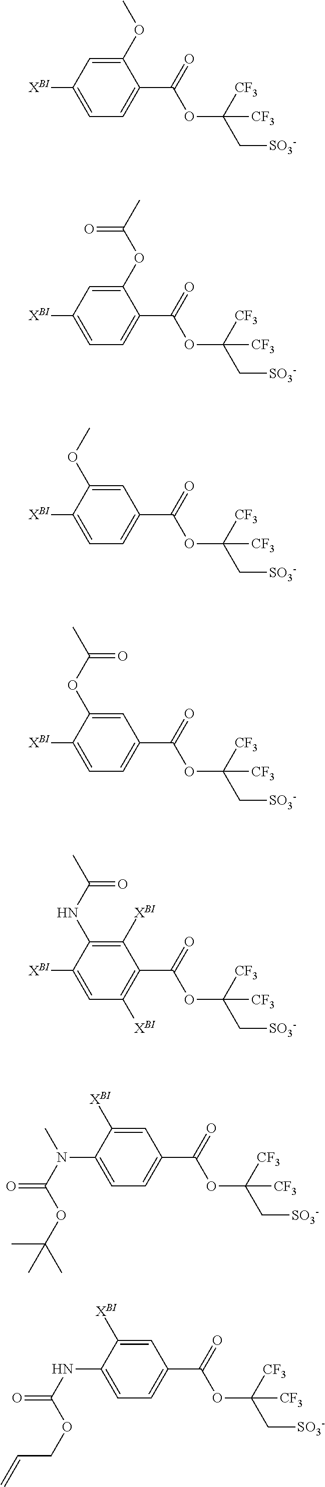

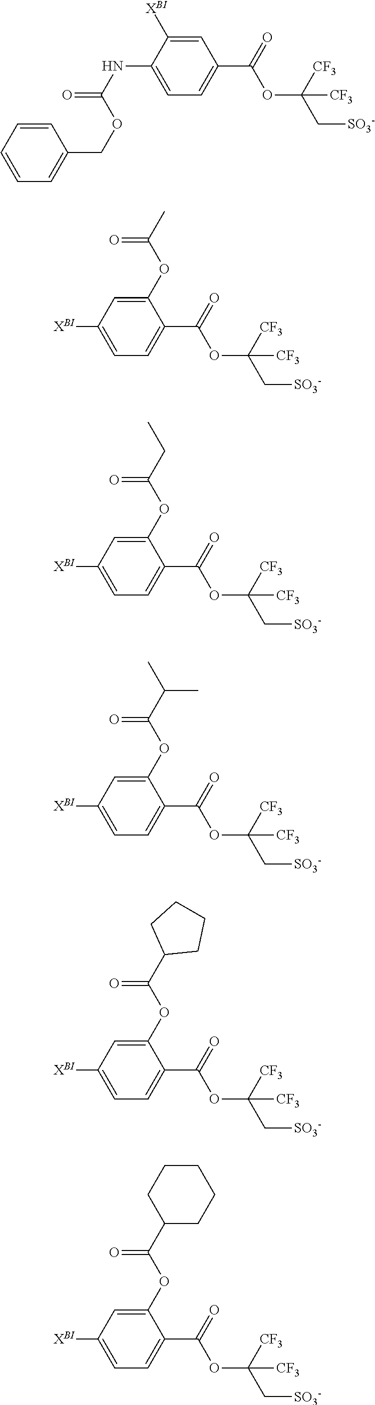

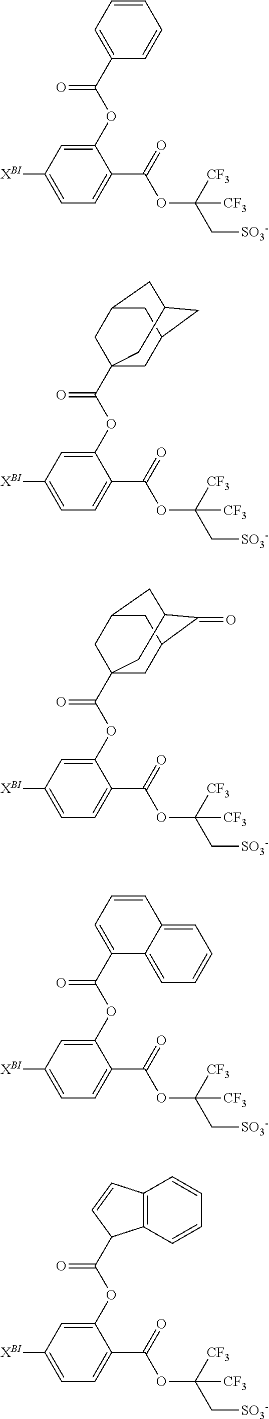

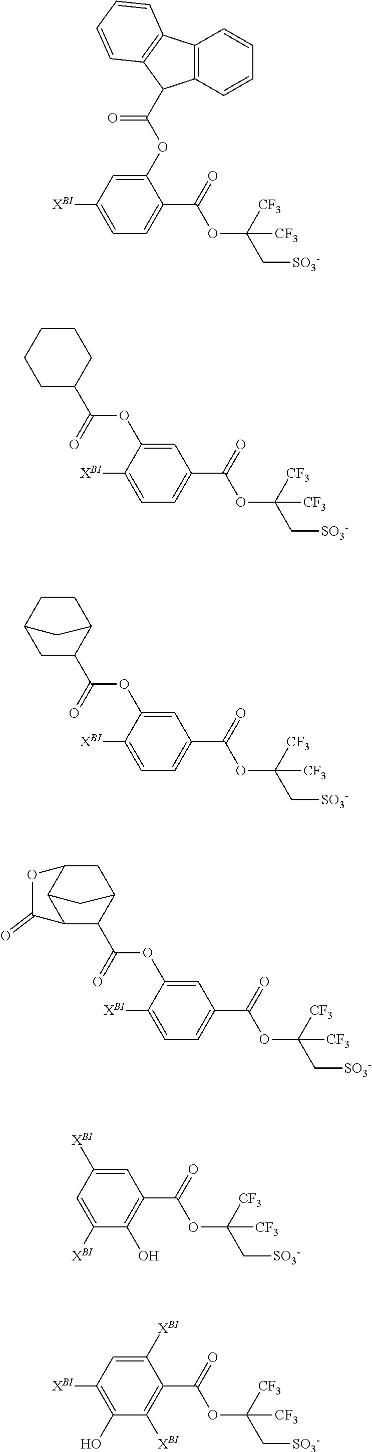

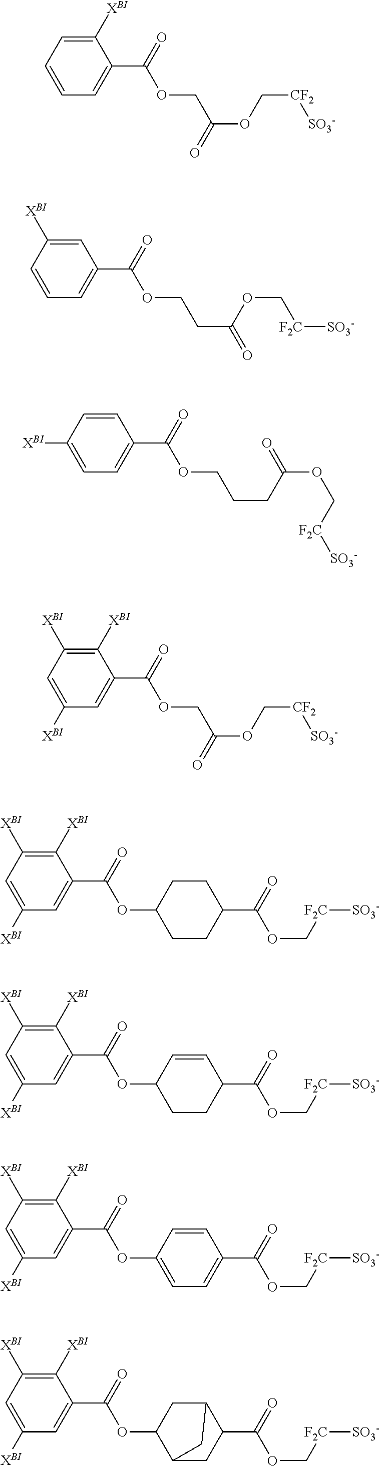

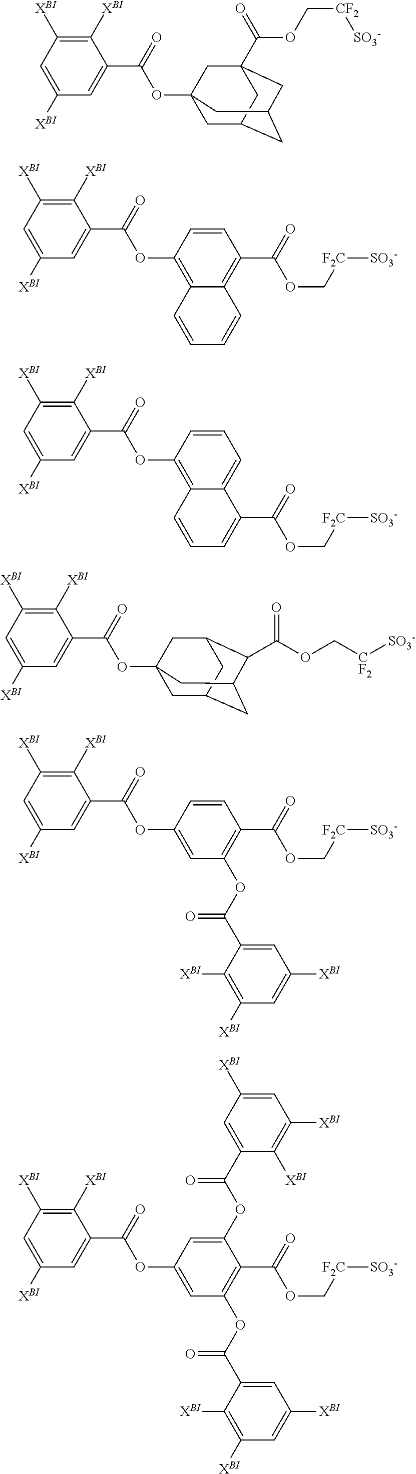

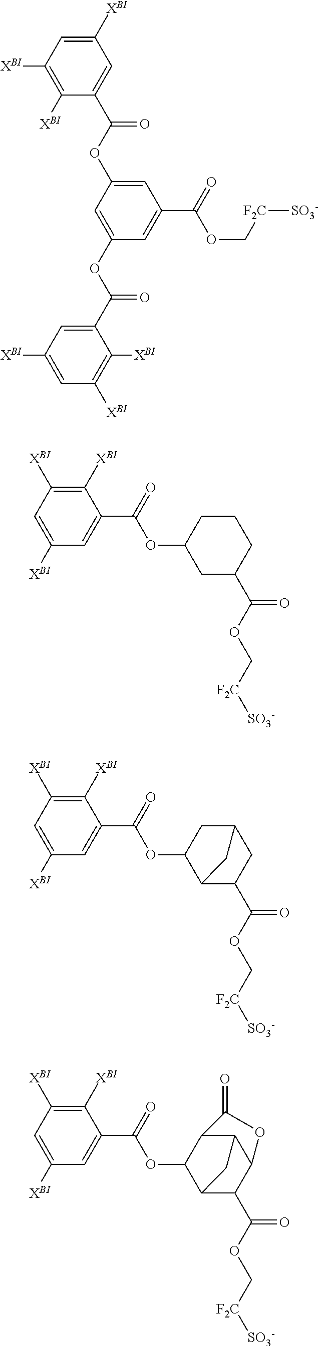

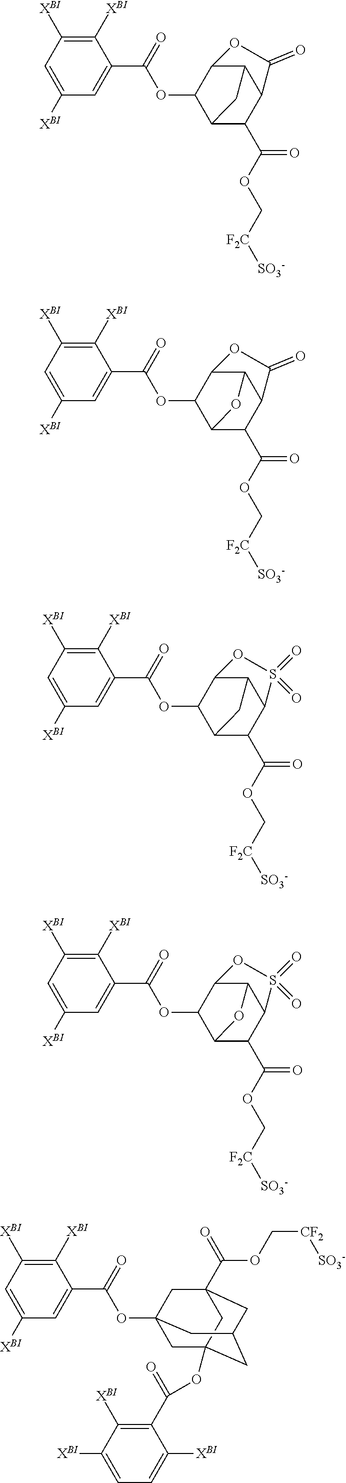

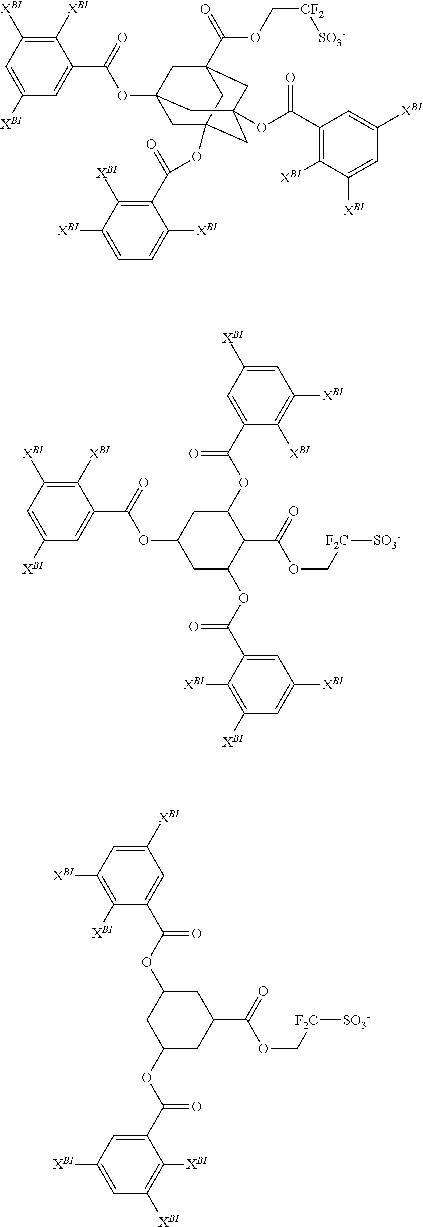

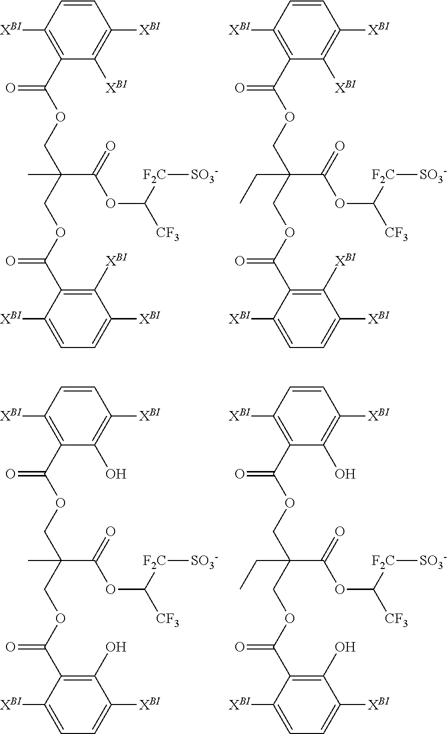

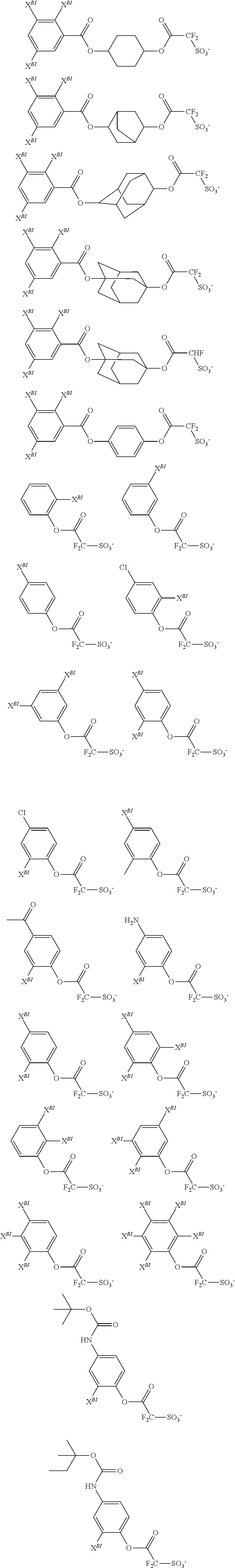

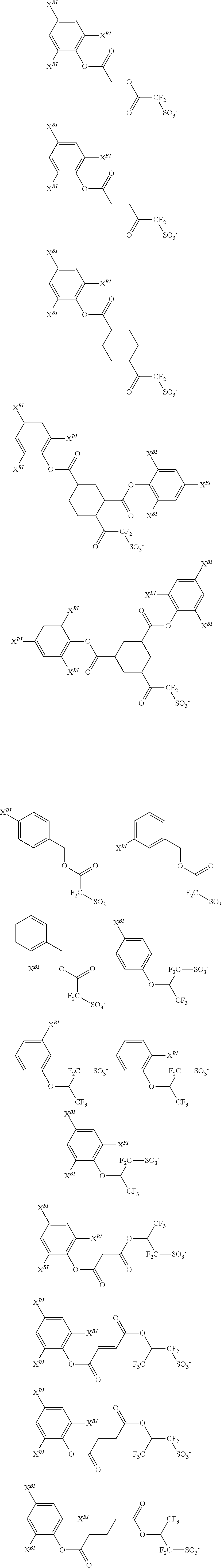

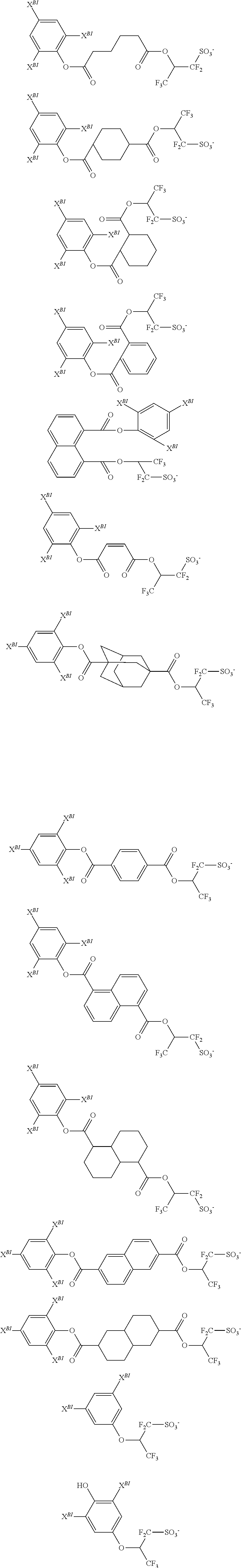

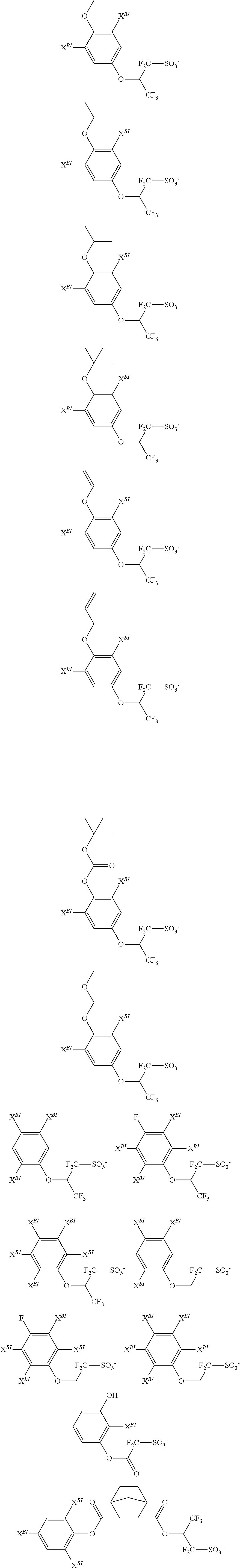

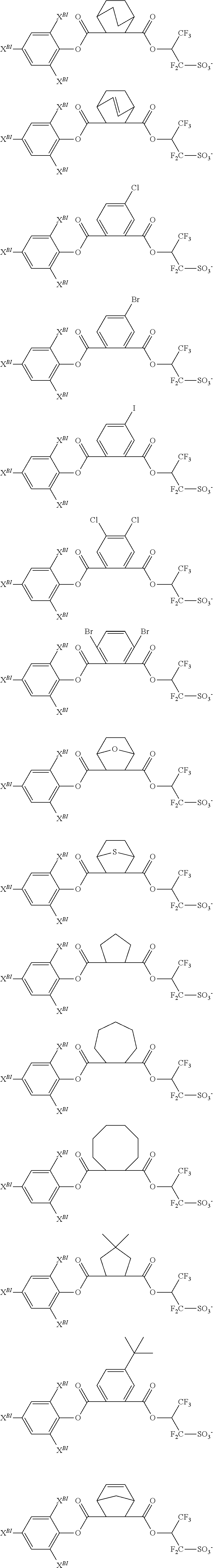

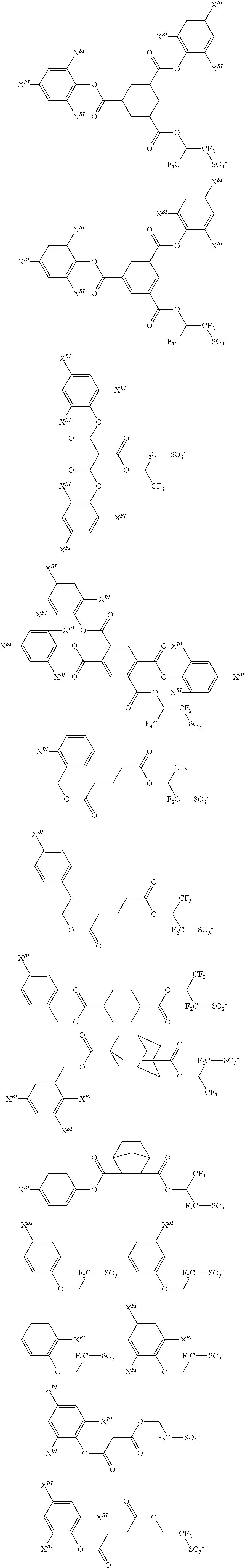

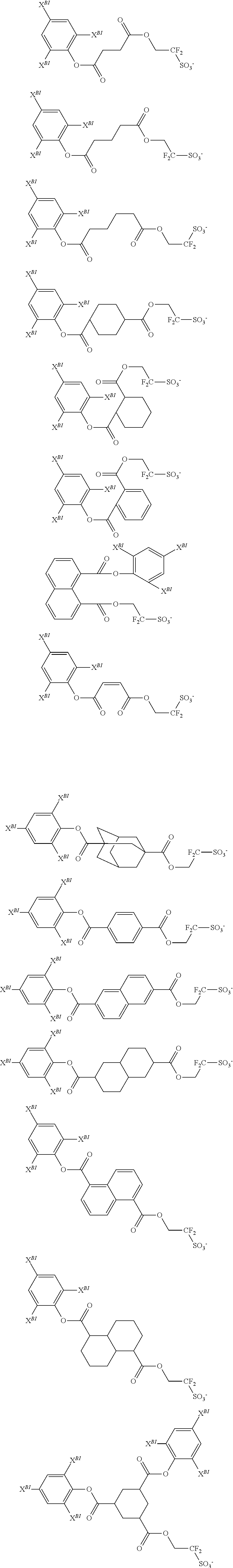

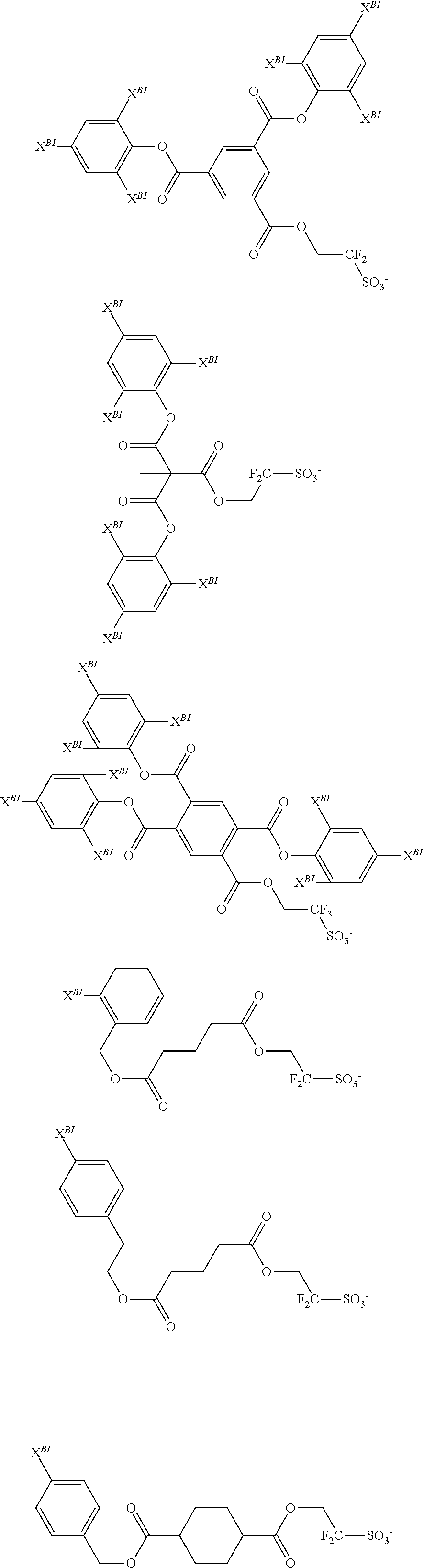

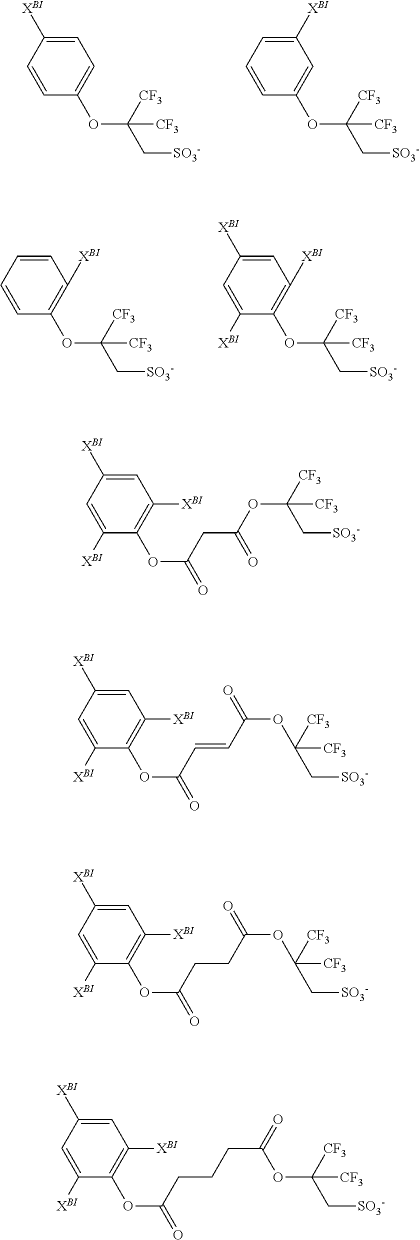

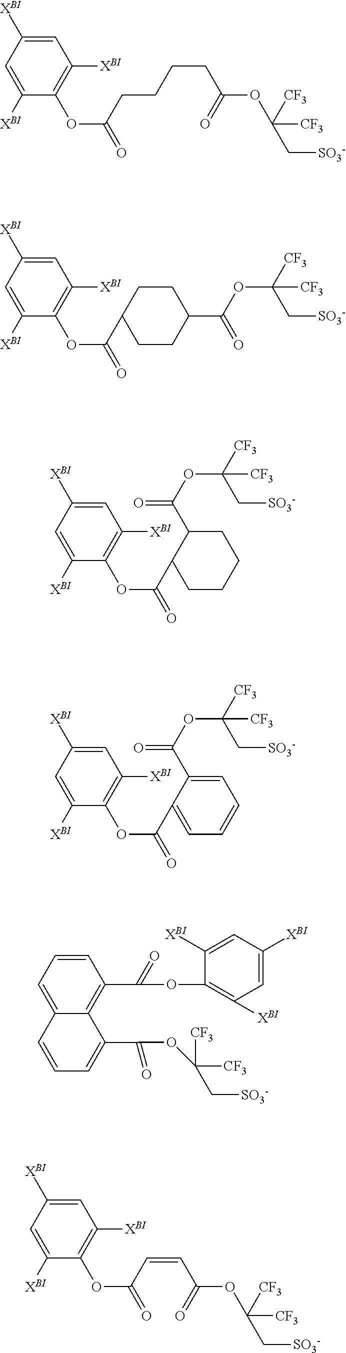

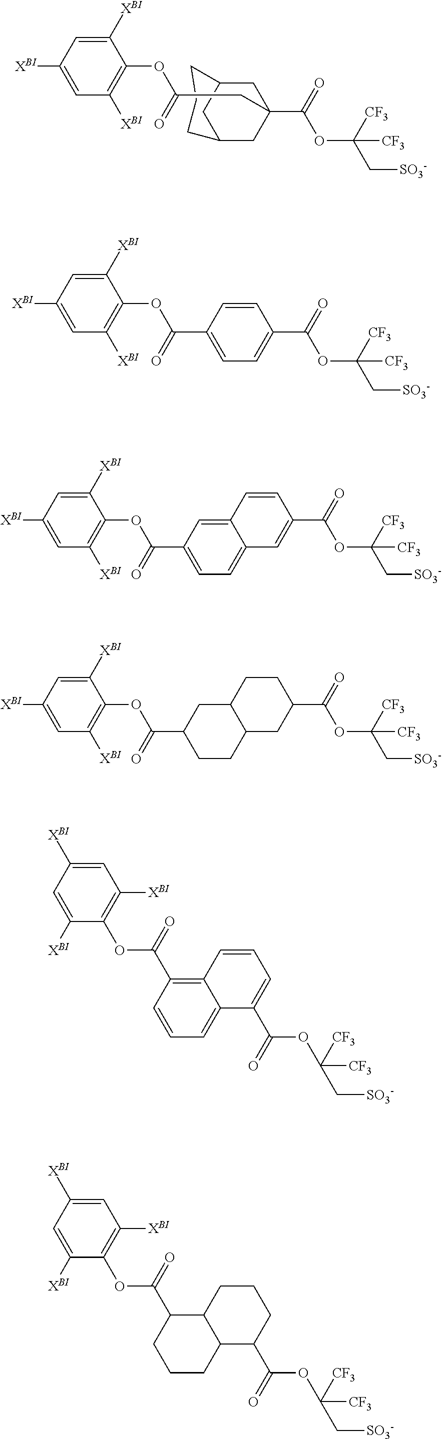

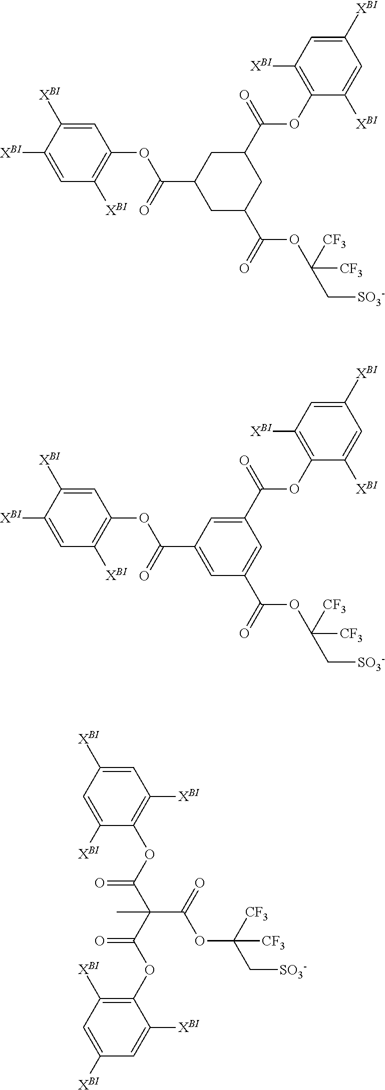

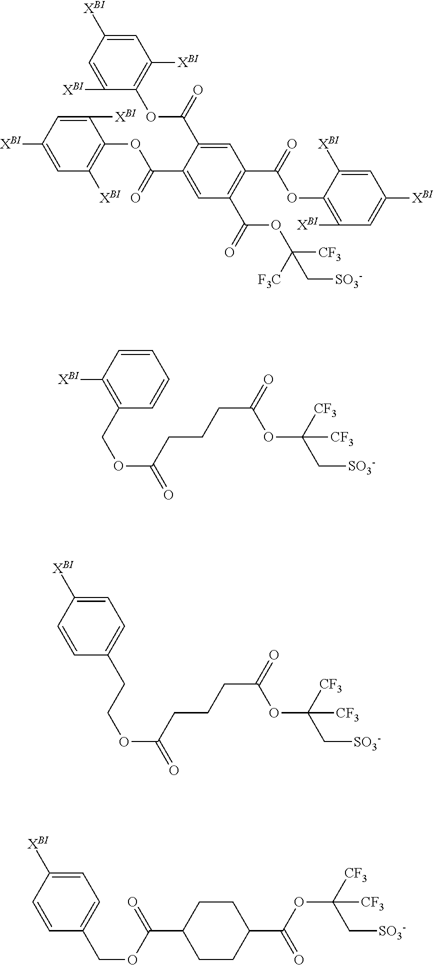

Other examples of the anion of Xa⁻ include those described in paragraphs [0076] and [0106] of WO 2023/157455, paragraph [0111] of WO 2024/24801, paragraphs [0253] to [0256] of WO 2024/43121, paragraphs [0044] and [0045] of WO 2024/57751, paragraphs [0205] to [0220] of WO 2024/122423, paragraphs [0170] to [0178] of JP-A 2023-123183, paragraphs [0026] to [0028] of JP-A 2024-62406, paragraphs [0022] to [0025] of JP-A 2024-62407, paragraphs [0026] to [0028] of JP-A 2024-62408, paragraphs [0028] to [0030] of JP-A 2024-68156, paragraphs [0026] to [0028] of JP-A 2024-68157, paragraphs [0028] to [0030] of JP-A 2024-68158, paragraphs [0028] to [0030] of JP-A 2024-68159, paragraphs [0031] to [0033] of JP-A 2024-72280, paragraphs [0023] to [0025] of JP-A 2024-72281, paragraphs [0026] to [0029] of JP-A 2024-77618, paragraphs [0020] and [0021] of JP-A 2024-77619, paragraphs [0140] to [0143] of JP-A 2024-80672, paragraphs [0023] to [0025] of JP-A 2024-83303, paragraphs [0028] to [0031] of JP-A 2024-83304, paragraphs [0030] to [0033] of JP-A 2024-99500, paragraphs [0028] to [0030] of JP-A 2024-99502, paragraphs [0030] to [0032] of JP-A 2024-101557, paragraphs [0025] to [0027] of JP-A 2024-102842, paragraphs [0033] to [0035] of JP-A 2024-102843, paragraphs [0021] and [0022] of JP-A 2024-127832, paragraphs [0169] to [0172] of JP-A 2024-144354, paragraphs [0178] to [0181] of JP-A 2024-144356, paragraphs [0040] to [0143] of JP-A 2024-160436, paragraphs [0157] and [0158] of JP 7247732, paragraphs [0227] to [0238] of JP 7446352, paragraphs [0253] to [0256] of JP 7466597, and paragraphs [0309] to [0312] of JP 7466782.

As the non-nucleophilic counter ion, a fluorobenzenesulfonic acid anion bonded to an aromatic group containing an iodine atom described in JP 6648726, an anion having a mechanism of decomposition by an acid described in WO 2021/200056 or JP-A 2021-70692, an anion having a cyclic ether group described in JP-A 2018-180525 or JP-A 2021-35935, and an anion described in JP-A 2018-92159 can also be used.

As the non-nucleophilic counter ion, a bulky benzenesulfonic acid derivative anion free of a fluorine atom described in JP 2006-A-276759, JP-A 2015-117200, JP-A 2016-65016, JP-A 2019-202974, or JP-A 2024-104830, and a benzenesulfonic acid anion or an alkylsulfonic acid anion free of a fluorine atom bonded to an aromatic group containing an iodine atom described in JP 6645464 can also be used. Anions described in paragraphs [0229] to [0231] of JP-A 2024-77330 and paragraphs [0033] to [0093] of JP-A 2024-140135 can also be used.

As the non-nucleophilic counter ion, an anion of a bissulfonic acid described in JP-A 2015-206932, an anion having a sulfonic acid on one side and having a sulfonamide or a sulfonimide different from the sulfonic acid on the other side, described in WO 2020/158366, and an anion having a sulfonic acid on one side and having a carboxylic acid on the other side, described in JP-A 2015-24989 can be further used.

As the photoacid generator as the component (D), those having the following formula (5) are also preferable.

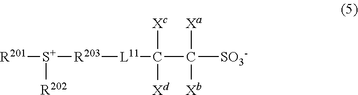

In the formula (5), R²⁰¹ and R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹, R²⁰², and R²⁰³ may be bonded to each other to form a ring together with a sulfur atom to which they are bonded.

The C₁-C₃₀ hydrocarbyl group of R²⁰¹ and R²⁰² may be saturated or unsaturated, and may be linear, branched, or cyclic. Specific examples thereof include: a C₁-C₃₀ alkyl group such as a methyl group, an ethyl group, a n-propyl group, an isopropyl group, a n-butyl group, an isobutyl group, a sec-butyl group, a tert-butyl group, a tert-pentyl group, a n-pentyl group, a n-hexyl group, a n-octyl group, a 2-ethylhexyl group, a n-nonyl group, or a n-decyl group; a C₃-C₃₀ cyclic saturated hydrocarbyl group such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, an oxanorbornyl group, a tricyclo[5.2.1.0^{2,6}] decyl group, or an adamantyl group; a C₆-C₃₀ aryl group such as a phenyl group, a methylphenyl group, an ethylphenyl group, a n-propylphenyl group, an isopropylphenyl group, a n-butylphenyl group, an isobutylphenyl group, a sec-butylphenyl group, a tert-butylphenyl group, a naphthyl group, a methylnaphthyl group, an ethylnaphthyl group, a n-propylnaphthyl group, an isopropylnaphthyl group, a n-butylnaphthyl group, an isobutylnaphthyl group, a sec-butylnaphthyl group, a tert-butylnaphthyl group, or an anthracenyl group; and a group obtained by combining these groups. Some or all of hydrogen atoms of the hydrocarbyl group may be replaced with a group containing a heteroatom such as an oxygen atom, a sulfur atom, a nitrogen atom, or a halogen atom, some of (—CH₂—)s in the hydrocarbyl group may be replaced with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic acid anhydride (—C(—O)—O—C(═O)—), a haloalkyl group, or the like.

The C₁-C₃₀ hydrocarbylene group of R²⁰³ may be saturated or unsaturated, and may be linear, branched, or cyclic. Specific examples thereof include: a C₁-C₃₀ alkanediyl group such as a methanediyl group, an ethane-1,1-diyl group, an ethane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, or a heptadecane-1,17-diyl group; a C₃-C₃₀ cyclic saturated hydrocarbylene group such as a cyclopentanediyl group, a cyclohexanediyl group, a norbornanediyl group, or an adamantanediyl group; and an arylene group such as a phenylene group, a methylphenylene group, an ethylphenylene group, a n-propylphenylene group, an isopropylphenylene group, a n-butylphenylene group, an isobutylphenylene group, a sec-butylphenylene group, a tert-butylphenylene group, a naphthylene group, a methylnaphthylene group, an ethylnaphthylene group, a n-propylnaphthylene group, an isopropylnaphthylene group, a n-butylnaphthylene group, an isobutylnaphthylene group, a sec-butylnaphthylene group, or a tert-butylnaphthylene group. Some or all of hydrogen atoms of the hydrocarbylene group may be replaced with a group containing a heteroatom such as an oxygen atom, a sulfur atom, a nitrogen atom, or a halogen atom, some of (—CH₂—)s in the hydrocarbylene group may be replaced with a group containing a heteroatom such as an oxygen atom, a sulfur atom, or a nitrogen atom, and as a result, the hydrocarbylene group may contain a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like. The heteroatom is preferably an oxygen atom.

In the formula (5), L²¹ is a single bond, an ether bond, or a C₁-C₂₀ hydrocarbylene group which may contain a heteroatom. The hydrocarbylene group may be saturated or unsaturated, and may be linear, branched, or cyclic. Specific examples thereof include those similar to those exemplified as the hydrocarbylene group of R²⁰³.

In the formula (5), X^{a}, X^{b}, X^{c}, and X^{d} are each independently a hydrogen atom, a fluorine atom, or a trifluoromethyl group. Note that at least one of X^{a}, X^{b}, X^{c}, and X^{d} is a fluorine atom or a trifluoromethyl group.

The photoacid generator having the formula (5) preferably has the following formula (5′):

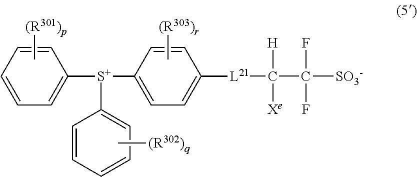

In the formula (5′), L²¹ is as defined above. Xe is a hydrogen atom or a trifluoromethyl group, and preferably a trifluoromethyl group. R³⁰¹, R³⁰², and R³⁰³ are each independently a hydrogen atom or a C₁-C₂₀ hydrocarbyl group which may contain a heteroatom. The hydrocarbyl group may be saturated or unsaturated, and may be linear, branched, or cyclic. Specific examples thereof include those similar to those exemplified as the hydrocarbyl group of R^{fa1} in the formula (Xa-1-1). p and q are each independently 0, 1, 2, 3, 4, or 5, and r is 0, 1, 2, 3, or 4.

Specific examples of the photoacid generator having the formula (5) include those similar to those exemplified as the photoacid generator having the formula (2) in JP-A 2017-26980.

Among the photoacid generators, those containing an anion having the formula (Xa-1-1) or (Xa-4) are particularly preferable because they have small acid diffusion and excellent solubility in a solvent. In addition, those having the formula (5′) have extremely small acid diffusion, and are particularly preferable.

When the chemically amplified resist composition of the present invention contains the photoacid generator (D), the content thereof is preferably 0.1 to 40 parts by weight, and more preferably 0.5 to 20 parts by weight per 80 parts by weight of the base polymer (A). When the addition amount of the photoacid generator (D) is in the above range, resolution is good, and there is no possibility that a problem of foreign matters occurs after development of a resist film or during peeling of the resist film, which is preferable. The photoacid generator (D) may be used singly or in combination of two or more types thereof.

### (E) Surfactant

The chemically amplified resist composition of the present invention may further contain a surfactant as a component (E). The surfactant (E) is preferably a surfactant insoluble or hardly soluble in water and soluble in an alkaline developer, or a surfactant insoluble or hardly soluble in water and an alkaline developer. As such a surfactant, those described in JP-A 2010-215608 and JP-A 2011-16746 can be referred to.

As the surfactant insoluble or hardly soluble in water and an alkaline developer, among the surfactants described in the above publications, FC-4430 (manufactured by 3M), SURFLON® S-381 (manufactured by AGC Seimi Chemical Co., Ltd.), OLFIN® E1004 (manufactured by Nissin Chemical Industry Co., Ltd.), KH-20 and KH-30 (manufactured by AGC Seimi Chemical Co., Ltd.), an oxetane ring-opened polymer having the following formula (surf-1), and the like are preferable.

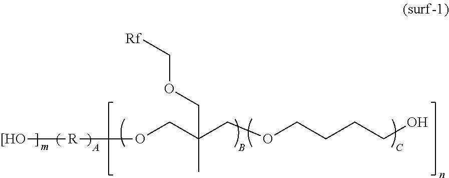

Here, R, Rf, A, B, C, m, n apply only to formula (surf-1) regardless of the above description. R is a di- to tetra-valent C₂-C₅ aliphatic group. Examples of the divalent aliphatic group include an ethylene group, a 1,4-butylene group, a 1,2-propylene group, a 2,2-dimethyl-1,3-propylene group, and a 1,5-pentylene group, and examples of the trivalent or tetravalent aliphatic group include the following groups:

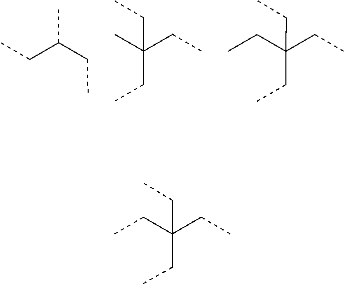

wherein broken lines are bonds, and are partial structures derived from glycerol, trimethylolethane, trimethylolpropane, and pentaerythritol, respectively.

Among these, a 1,4-butylene group, a 2,2-dimethyl-1,3-propylene group, and the like are preferable.

Rf is a trifluoromethyl group or a pentafluoroethyl group, and preferably a trifluoromethyl group. m is an integer of 0 to 3, n is an integer of 1 to 4, and a sum of n and m is a valence of R and an integer of 2 to 4. A is 1. B is an integer of 2 to 25, and preferably an integer of 4 to 20. C is an integer of 0 to 10, and preferably 0 or 1. Constituent units in the formula (surf-1) do not define an arrangement thereof, and may be bonded in a block manner or randomly. Manufacture of a partially fluorinated oxetane ring-opened polymer-based surfactant is described in detail in U.S. Pat. No. 5,650,483 and the like.

When a resist protective film is not used in ArF immersion lithography, a surfactant insoluble or hardly soluble in water and soluble in an alkaline developer has a function of reducing water penetration and leaching by being oriented on a surface of a resist film. Therefore, the surfactant insoluble or hardly soluble in water and soluble in an alkaline developer is useful for suppressing elution of a water-soluble component from the resist film to reduce damage to an exposure apparatus, and is also useful because it is solubilized during alkali aqueous solution development after exposure or after post exposure bake (PEB), and hardly becomes a foreign matter that causes defects. Such a surfactant is a polymeric surfactant having a property of being insoluble or hardly soluble in water and being soluble in an alkaline developer, is also called a hydrophobic resin, and is particularly preferably a surfactant having high water repellency and improving water slippability.

Specific examples of such a polymeric surfactant include those containing at least one selected from a repeat unit having the following formula (6A) (hereinafter, also referred to as a repeat unit 6A), a repeat unit having the following formula (6B) (hereinafter, also referred to as a repeat unit 6B), a repeat unit having the following formula (6C) (hereinafter, also referred to as a repeat unit 6C), a repeat unit having the following formula (6D) (hereinafter, also referred to as a repeat unit 6D), and a repeat unit having the following formula (6E) (hereinafter, also referred to as a repeat unit 6E).

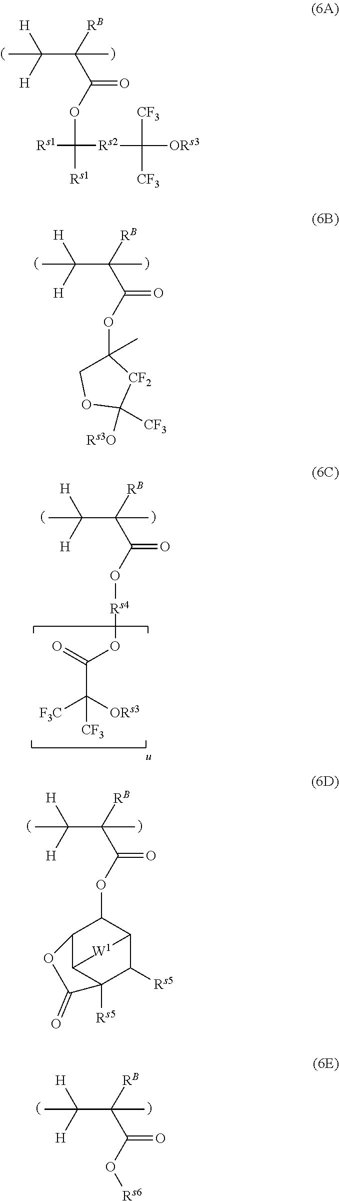

In the formulas (6A) to (6E), R^{B} is a hydrogen atom, a fluorine atom, a methyl group, or a trifluoromethyl group. W¹ is-CH₂—, —CH₂CH₂—, —O—, or two (—H) s separated from each other. R^{s1}s are each independently a hydrogen atom or a C₁-C₁₀ hydrocarbyl group. R^{s2} is a single bond or a linear or branched C₁-C₈ hydrocarbylene group. R^{s3}s are each independently a hydrogen atom, a C₁-C₁₅ hydrocarbyl group or fluorinated hydrocarbyl group, or an acid-unstable group. When R^{s3} is a hydrocarbyl group or a fluorinated hydrocarbyl group, an ether bond or a carbonyl group may be interposed between a carbon-carbon bond. R^{s4} is a (u+1)-valent C₁-C₂₀ hydrocarbon group or fluorinated hydrocarbon group. u is 1, 2, or 3. R^{s5}s are each independently a hydrogen atom or a group of—C(═O)—O—R^{sa}. R^{sa} is a C₁-C₂₀ fluorinated hydrocarbyl group. R^{s6} is a C₁-C₁₅ hydrocarbyl group or fluorinated hydrocarbyl group, and an ether bond or a carbonyl group may be interposed between a carbon-carbon bond thereof.

The C₁-C₁₀ hydrocarbyl group of R^{s1} is preferably a saturated hydrocarbyl group, and may be linear, branched, or cyclic. Specific examples thereof include: a C₁-C₁₀ alkyl group such as a methyl group, an ethyl group, a n-propyl group, an isopropyl group, a n-butyl group, an isobutyl group, a sec-butyl group, a tert-butyl group, a n-pentyl group, a n-hexyl group, a n-heptyl group, a n-octyl group, a n-nonyl group, or a n-decyl group; and a C₃-C₁₀ cyclic saturated hydrocarbyl group such as a cyclopropyl group, a cyclobutyl group, a cyclopentyl group, a cyclohexyl group, an adamantyl group, or a norbornyl group. Among these, groups having 1 to 6 carbon atoms are preferable.

The hydrocarbylene group of R^{s2} is preferably a saturated hydrocarbylene group, and may be linear, branched, or cyclic. Specific examples thereof include a methylene group, an ethylene group, a propylene group, a butylene group, and a pentylene group.

The hydrocarbyl group of R^{s3} or R^{s6} may be saturated or unsaturated, and may be linear, branched, or cyclic. Specific examples thereof include a saturated hydrocarbyl group, an aliphatic unsaturated hydrocarbyl group such as an alkenyl group or an alkynyl group, and a saturated hydrocarbyl group is preferable. Specific examples of the saturated hydrocarbyl group include an undecyl group, a dodecyl group, a tridecyl group, a tetradecyl group, and a pentadecyl group in addition to those exemplified as the hydrocarbyl group of R^{s1}. Specific examples of the fluorinated hydrocarbyl group of R^{s3} or R^{s6} include groups in which some or all of hydrogen atoms bonded to carbon atoms of the above-described hydrocarbyl group are replaced with fluorine atoms. As described above, an ether bond or a carbonyl group may be interposed between a carbon-carbon bond of these groups.

Specific examples of the acid-unstable group of R^{s3} include groups having the formulas (AL-3) to (AL-5) described above, a trialkylsilyl group in which each alkyl group is a C₁-C₆ alkyl group, and a C₄-C₂₀ oxo group-containing alkyl group.

The (u+1)-valent hydrocarbon group or fluorinated hydrocarbon group of R^{s4} may be linear, branched, or cyclic, and specific examples thereof include a group obtained by further eliminating u hydrogen atoms from the above-described hydrocarbyl group, fluorinated hydrocarbyl group, or the like.

The fluorinated hydrocarbyl group of R^{sa} is preferably saturated, and may be linear, branched, or cyclic. Specific examples thereof include groups in which some or all of hydrogen atoms of the hydrocarbyl group are replaced with fluorine atoms, and specific examples thereof include a trifluoromethyl group, a 2,2,2-trifluoroethyl group, a 3,3,3-trifluoro-1-propyl group, a 3,3,3-trifluoro-2-propyl group, a 2,2,3,3-tetrafluoropropyl group, a 1,1,1,3,3,3-hexafluoroisopropyl group, a 2,2,3,3,4,4,4-heptafluorobutyl group, a 2,2,3,3,4,4,5,5-octafluoropentyl group, a 2,2,3,3,4,4,5,5,6,6,7,7-dodecafluoroheptyl group, a 2-(perfluorobutyl) ethyl group, a 2-(perfluorohexyl) ethyl group, a 2-(perfluorooctyl) ethyl group, and a 2-(perfluorodecyl) ethyl group.

Specific examples of the repeat units 6A to 6E include, but are not limited to, those listed below. Note that, in the following formulas, R^{B} is as defined above.

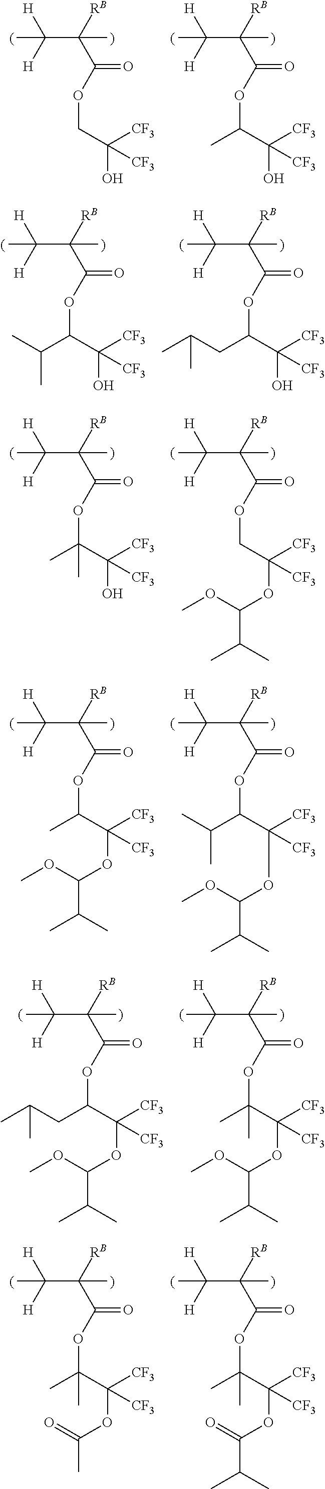

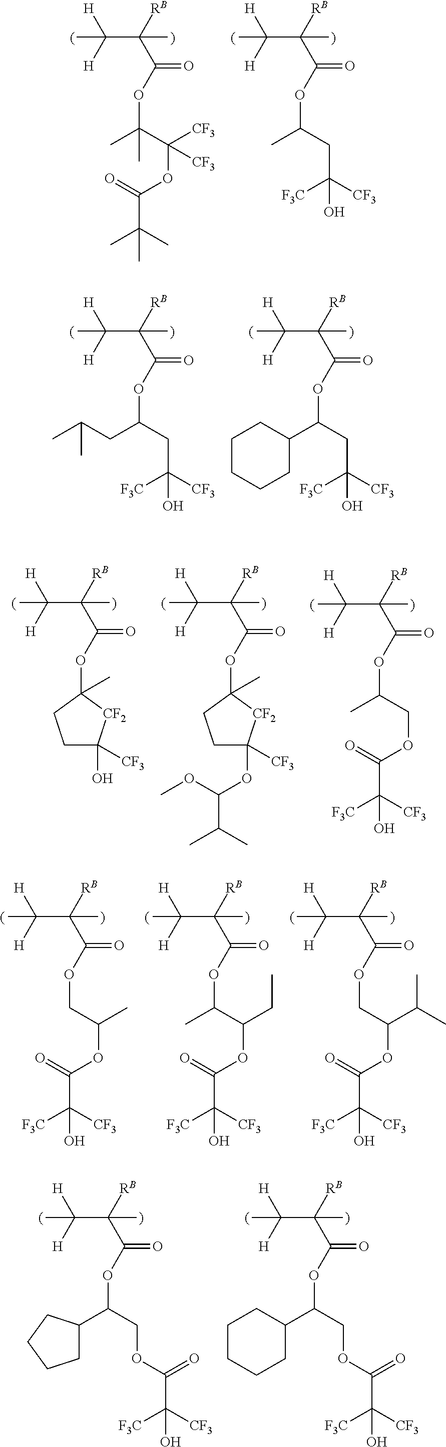

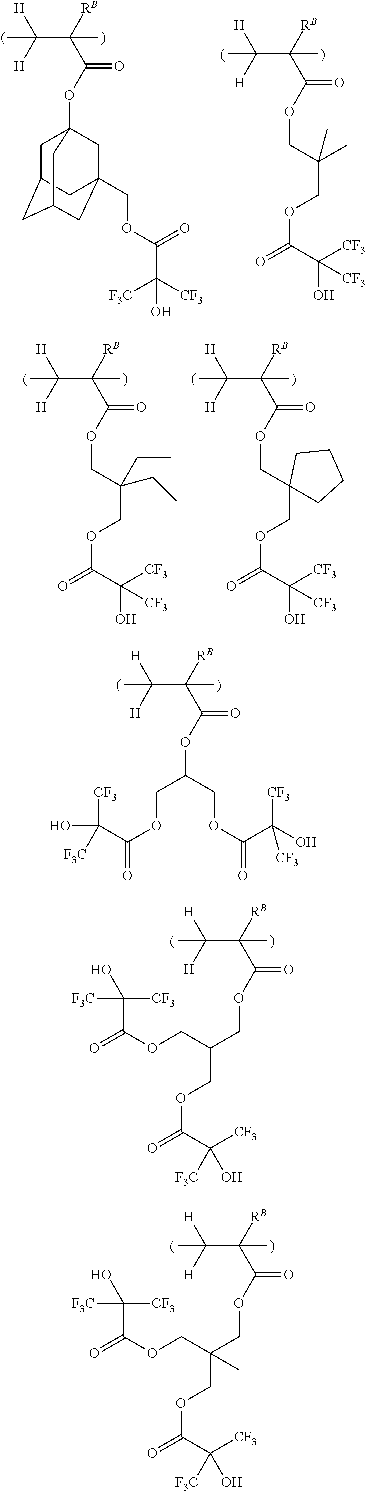

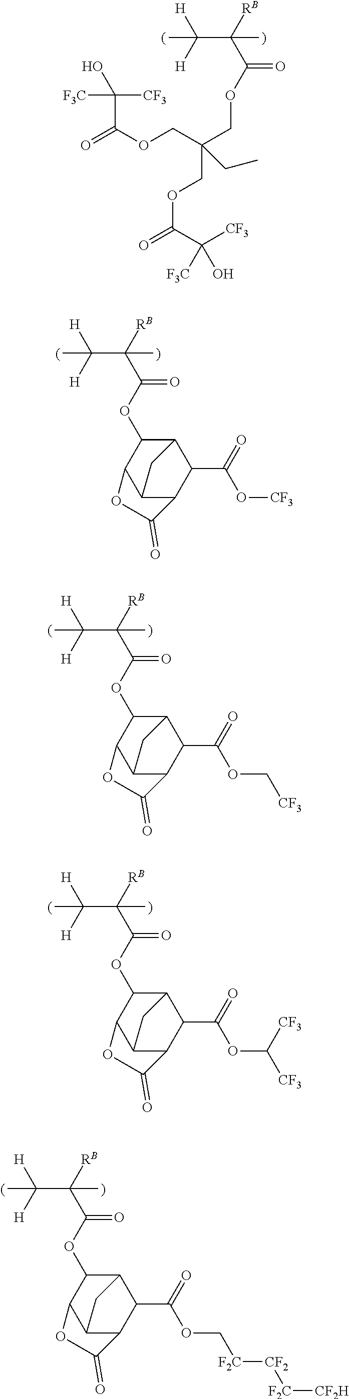

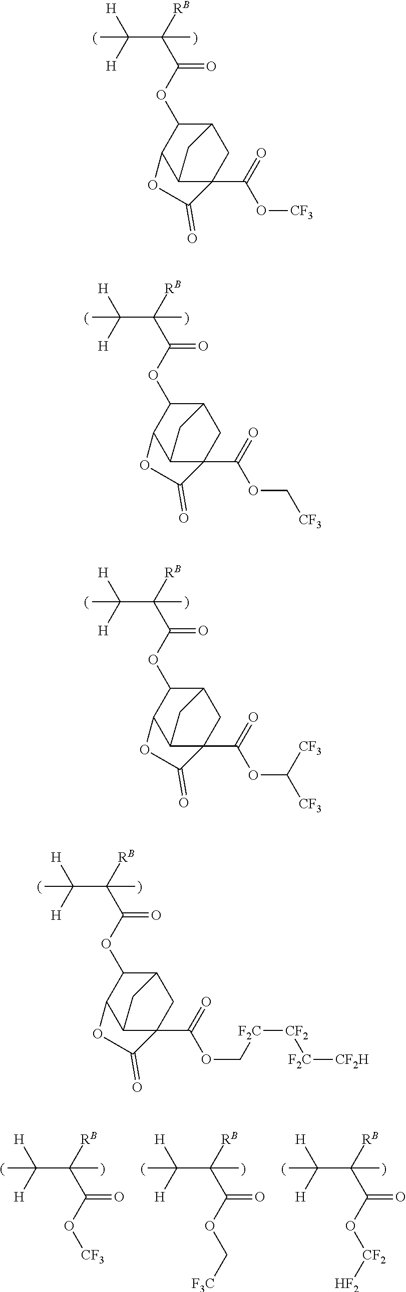

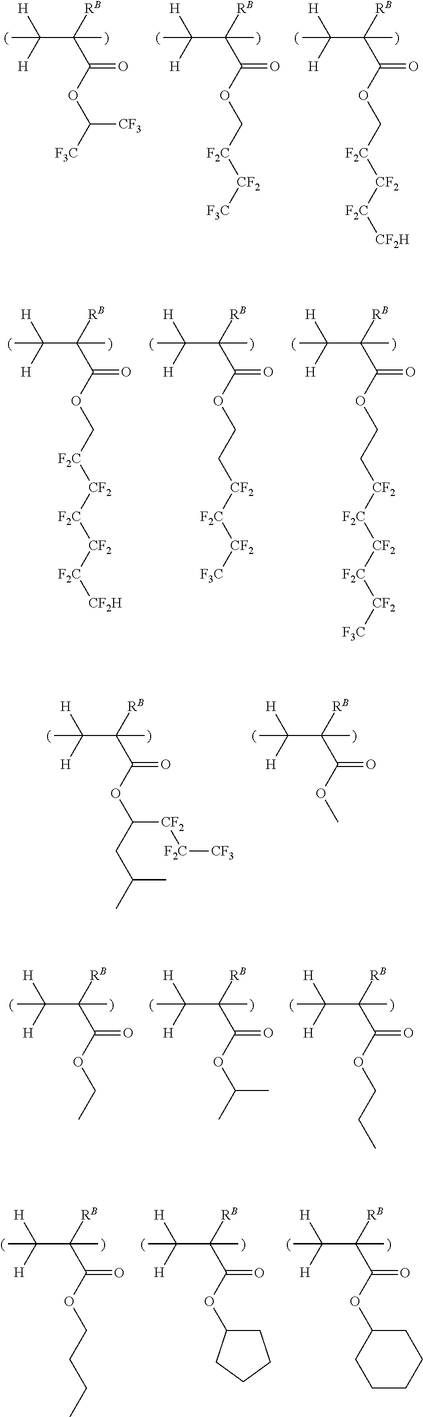

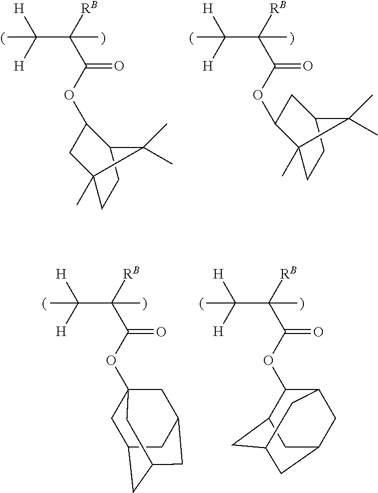

The polymeric surfactant may further contain a repeat unit other than the repeat units 6A to 6E. Specific examples of the other repeat unit include a repeat unit obtained from methacrylic acid, an α-trifluoromethylacrylic acid derivative, or the like. The content of the repeat units 6A to 6E in the polymeric surfactant is preferably 20 mol % or more, more preferably 60 mol % or more, and still more preferably 100 mol % in all repeat units.

Mw of the polymeric surfactant is preferably 1000 to 500,000, and more preferably 3000 to 100,000. Mw/Mn is preferably 1.0 to 2.0, and more preferably 1.0 to 1.6.

Examples of a method for synthesizing the polymeric surfactant include a method in which a monomer containing at least one selected from the repeat units 6A to 6E and if necessary, an unsaturated bond that provides another repeat unit is heated in an organic solvent together with a radical initiator to be polymerized. Specific examples of an organic solvent used during the polymerization include toluene, benzene, THF, diethyl ether, and dioxane. Specific examples of a polymerization initiator include AIBN, 2,2′-azobis(2,4-dimethylvaleronitrile), dimethyl 2,2-azobis(2-methylpropionate), benzoyl peroxide, and lauroyl peroxide. A reaction temperature is preferably 50 to 100° C. A reaction time is preferably 4 to 24 hours. An acid-unstable group introduced into the monomer may be used as it is, or protection or partial protection may be performed after polymerization.

When the polymeric surfactant is synthesized, a known chain transfer agent such as dodecyl mercaptan or 2-mercaptoethanol may be used in order to adjust a molecular weight. In this case, the addition amount of these chain transfer agents is preferably 0.01 to 10 mol % based on the total moles of monomers to be polymerized.

When the chemically amplified resist composition of the present invention contains the surfactant (E), the content thereof is preferably 0.1 to 50 parts by weight, and more preferably 0.5 to 10 parts by weight per 80 parts by weight of the base polymer (A). When the content of the surfactant (E) is 0.1 parts by weight or more, a receding contact angle between a surface of a resist film and water is sufficiently improved. When the content of the surfactant (E) is 50 parts by weight or less, a dissolution rate of the surface of the resist film in a developer is low, and the height of a formed fine pattern is sufficiently maintained. The surfactant (E) may be used singly or in combination of two or more types thereof.

### (F) Dissolution Inhibitor

The chemically amplified resist composition of the present invention may further contain a dissolution inhibitor as a component (F). When the chemically amplified resist composition of the present invention is a positive type, by blending a dissolution inhibitor, a difference in dissolution rate between an exposed portion and an unexposed portion can be further increased, and resolution can be further improved.

Specific examples of the dissolution inhibitor include a compound having a molecular weight of preferably 100 to 1000, more preferably 150 to 800, and containing two or more phenolic hydroxy groups in a molecule thereof, in which hydrogen atoms of the phenolic hydroxy groups are replaced with acid-unstable groups in a ratio of 0 to 100 mol % as a whole, and a compound containing a carboxy group in a molecule thereof, in which hydrogen atoms of the carboxy groups are replaced with acid-unstable groups in a ratio of 50 to 100 mol % on average as a whole. Specific examples thereof include bisphenol A, trisphenol, phenolphthalein, cresol novolac, naphthalenecarboxylic acid, adamantanecarboxylic acid, and a compound in which a hydroxy group of cholic acid and a hydrogen atom of a carboxy group are replaced with acid-unstable groups, and examples thereof include those described in paragraphs [0155] to [0178] of JP-A 2008-122932.

When the chemically amplified resist composition of the present invention contains a dissolution inhibitor (F), the content thereof is preferably 0 to 50 parts by weight, and more preferably 5 to 40 parts by weight per 80 parts by weight of the base polymer (A). The dissolution inhibitor (F) may be used singly or in combination of two or more types thereof.

### (G) Other Components

The chemically amplified resist composition of the present invention may contain, as other components (G), a compound that is decomposed by an acid to generate an acid (acid generation compound), an organic acid derivative, a fluorine-substituted alcohol, a water repellency improver, and the like. As the acid generation compound, a compound described in JP-A 2009-269953 or JP-A 2010-215608 can be referred to. When the chemically amplified resist composition of the present invention contains the acid generation compound, the content thereof is preferably 0 to 5 parts by weight, and more preferably 0 to 3 parts by weight per 80 parts by weight of the base polymer (A). When the content is too large, it is difficult to control acid diffusion, and resolution and a pattern shape may be deteriorated. As the organic acid derivative and the fluorine-substituted alcohol, a compound described in JP-A 2009-269953 or JP-A 2010-215608 can be referred to.

The water repellency improver can be used for immersion lithography without using a topcoat. As the water repellency improver, a polymer containing a fluorinated alkyl group, a polymer containing a 1,1,1,3,3,3-hexafluoro-2-propanol residue having a specific structure, or the like is preferable, and those exemplified in JP-A 2007-297590, JP-A 2008-111103, and the like are more preferable. The water repellency improver needs to be dissolved in an alkaline developer or an organic solvent developer. The above-described water repellency improver having a 1,1,1,3,3,3-hexafluoro-2-propanol residue having a specific structure has good solubility in a developer. As the water repellency improver, a polymer containing a repeat unit containing an amino group or an amine salt has a high effect of preventing evaporation of an acid in PEB and preventing defective opening of a hole pattern after development. When the chemically amplified resist composition of the present invention contains the water repellency improver, the content thereof is preferably 0 to 20 parts by weight, and more preferably 0.5 to 10 parts by weight per 80 parts by weight of the base polymer (A).

### [Pattern Forming Method]

When the chemically amplified resist composition of the present invention is used for manufacturing various integrated circuits, a known lithography technique can be applied. Examples of a pattern forming method include a method including: a step of forming a resist film on a substrate using the above-described chemically amplified resist composition; a step of exposing the resist film to a high energy ray; and a step of developing the exposed resist film using a developer.

First, the chemically amplified resist composition of the present invention is applied onto a substrate for manufacturing an integrated circuit (Si, SiO₂, SIN, SiON, TiN, WSi, BPSG, SOG, an organic antireflection film, or the like) or a substrate for manufacturing a mask circuit (Cr, CrO, CrON, MoSi₂, SiO₂, or the like) by an appropriate coating method such as spin coating, roll coating, flow coating, dip coating, spray coating, or doctor coating so as to have a coating film thickness of 0.01 to 2.0 μm. This product is prebaked on a hot plate preferably at 60 to 150° C. for 10 seconds to 30 minutes, more preferably at 80 to 120° C. for 30 seconds to 20 minutes to form a resist film.

Next, the resist film is exposed to a high energy ray. Examples of the high energy ray include an ultraviolet ray, a far ultraviolet ray, EB, EUV having a wavelength of 3 to 15 nm, an X-ray, a soft X-ray, excimer laser light, a γ-ray, and a synchrotron radiation. When an ultraviolet ray, a far ultraviolet ray, EUV, an X-ray, a soft X-ray, excimer laser light, a γ-ray, a synchrotron radiation, or the like is used as the high energy ray, irradiation is performed directly or using a mask for forming a target pattern such that an exposure dose is preferably about 1 to 200 mJ/cm², and more preferably about 10 to 100 mJ/cm². When EB is used as the high energy ray, drawing is performed directly or using a mask for forming a target pattern such that an exposure dose is preferably about 0.1 to 100 μC/cm², and more preferably about 0.5 to 50 μC/cm². The chemically amplified resist composition of the present invention is particularly suitable for fine patterning with ArF excimer laser having a wavelength of 193 nm, KrF excimer laser having a wavelength of 248 nm, EB, EUV having a wavelength of 3 to 15 nm, an X-ray, a soft X-ray, a γ-ray, or a synchrotron radiation among high energy rays.

After exposure, PEB may be performed on a hot plate preferably at 60 to 150° C. for 10 seconds to 30 minutes, more preferably at 80 to 120° C. for 30 seconds to 20 minutes.

After exposure or PEB, development is performed by a conventional method such as a dip method, a puddle method, or a spray method for three seconds to three minutes, preferably five seconds to two minutes, using a 0.1 to 10 wt %, preferably 2 to 5 wt % of alkali aqueous solution of tetramethylammonium hydroxide (TMAH), tetraethylammonium hydroxide, tetrapropylammonium hydroxide, tetrabutylammonium hydroxide, or the like, whereby a portion irradiated with light is dissolved in the developer, an unexposed portion is not dissolved therein, and a target positive pattern is formed on the substrate.

A negative pattern can also be obtained using an organic solvent developer instead of the alkali aqueous solution. Specific examples of the developer used at this time include 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate. These organic solvents may be used singly or in combination of two or more types thereof.

At an end of development, rinsing may be performed. A rinse liquid is preferably a solvent that is mixed with the developer and does not dissolve the resist film. As such a solvent, a C₃-C₁₀ alcohol, a C₈-C₁₂ ether compound, a C₆-C₁₂ alkane, a C₆-C₁₂ alkene, a C₆-C₁₂ alkyne, and an aromatic solvent are preferably used.

Specific examples of the C₃-C₁₀ alcohol include n-propyl alcohol, isopropyl alcohol, 1-butyl alcohol, 2-butyl alcohol, isobutyl alcohol, tert-butyl alcohol, 1-pentanol, 2-pentanol, 3-pentanol, tert-pentyl alcohol, neopentyl alcohol, 2-methyl-1-butanol, 3-methyl-1-butanol, 3-methyl-3-pentanol, cyclopentanol, 1-hexanol, 2-hexanol, 3-hexanol, 2,3-dimethyl-2-butanol, 3,3-dimethyl-1-butanol, 3,3-dimethyl-2-butanol, 2-ethyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 4-methyl-3-pentanol, cyclohexanol, and 1-octanol.

Specific examples of the C₈-C₁₂ ether compound include di-n-butyl ether, diisobutyl ether, di-sec-butyl ether, di-n-pentyl ether, diisopentyl ether, di-sec-pentyl ether, di-tert-pentyl ether, and di-n-hexyl ether.

Specific examples of the C₆-C₁₂ alkane include hexane, heptane, octane, nonane, decane, undecane, dodecane, methylcyclopentane, dimethylcyclopentane, cyclohexane, methylcyclohexane, dimethylcyclohexane, cycloheptane, cyclooctane, and cyclononane.

Specific examples of the C₆-C₁₂ alkene include hexene, heptene, octene, cyclohexene, methylcyclohexene, dimethylcyclohexene, cycloheptene, and cyclooctene. Specific examples of the C₆-C₁₂ alkyne include hexyne, heptyne, and octyne.

Specific examples of the aromatic solvent include toluene, xylene, ethylbenzene, isopropylbenzene, tert-butylbenzene, and mesitylene.

By performing rinsing, it is possible to reduce occurrence of collapse and defects of a resist pattern. Rinsing is not necessarily required, and the amount of solvent used can be reduced by not performing rinsing.

A hole pattern and a trench pattern after development can also be shrunk by thermal flow, a RELACS technique, or a DSA technique. A shrink agent is applied onto the hole pattern, crosslinking of the shrink agent occurs on a surface of the resist film due to diffusion of an acid catalyst from the resist film during baking, and the shrink agent adheres to a side wall of the hole pattern. A baking temperature is preferably 70 to 180° C., and more preferably 80 to 170° C., and a baking time is preferably 10 to 300 seconds, whereby an excessive amount of the shrink agent is removed to reduce the hole pattern.

### EXAMPLES

Hereinafter, the present invention is described specifically with reference to Synthesis Examples, Examples, and Comparative Examples, but the present invention is not limited to the following Examples. Note that a device used is as follows.

MALDI TOF-MS: S3000 manufactured by JEOL Ltd.

### [1] Synthesis of Sulfonium Salt Monomer

### Example 1-1

### Synthesis of Sulfonium Salt Monomer a-1

### (1) Synthesis of Intermediate in-1

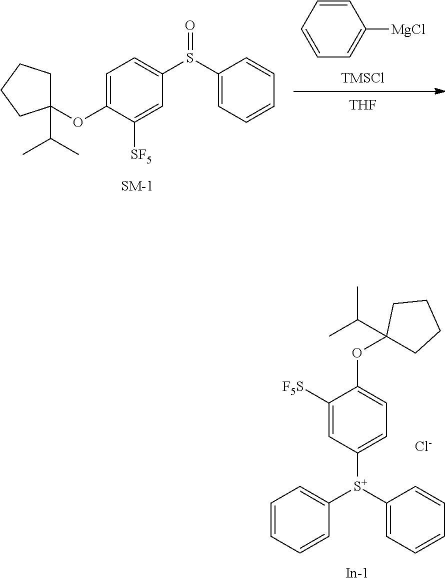

In a nitrogen atmosphere, a Grignard reagent was prepared by a conventional method using chlorobenzene (67.5 g), metallic magnesium (14.6 g), and THF (400 g). Thereafter, the reaction liquid was cooled to 10° C. or lower, and a solution containing a compound SM-1 (90.9 g) and THF (90 g) was added thereto. Trimethylsilyl chloride (65.2 g) was further added dropwise thereto at an internal temperature of 20° C. or lower. After the dropwise addition, the mixture was aged at an internal temperature of 20° C. or lower for three hours. After aging, a saturated ammonium chloride aqueous solution (500 g) was added dropwise thereto to stop the reaction. Thereafter, methylene chloride (500 g) was added thereto, and the organic layer was separated, washed with water, and then concentrated under reduced pressure to distill off the solvent. The residue was purified by silica gel column chromatography to obtain 89.3 g of an intermediate In-1 as a colorless oily product (yield: 81%).

### (2) Synthesis of Sulfonium Salt Monomer a-1

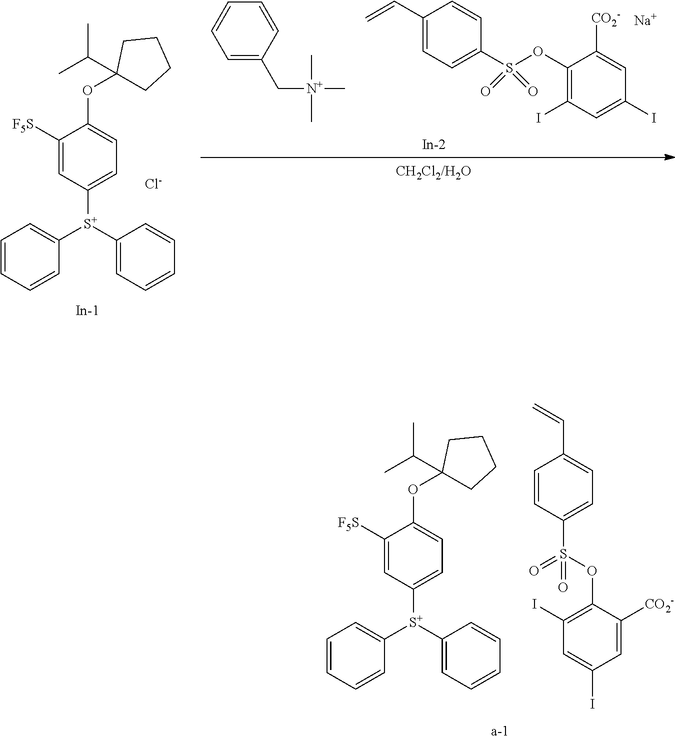

In a nitrogen atmosphere, the intermediate In-1 (55.1 g), an intermediate In-2 (57.8 g), methyl isobutyl ketone (300 g), and water (150 g) were put, and the mixture was stirred at room temperature for 30 minutes. The organic layer was separated, washed with water, and then concentrated under reduced pressure. The residue was purified by silica gel column chromatography to obtain 98.4 g of a monomer a-1 as an oily product (yield: 92%).

Results of TOF-MS of the sulfonium salt monomer a-1 are described below.

### MALDI TOF-MS:

POSITIVE M⁺515 (corresponding to C₂₆H₂₈F₅OS₂⁺)
NEGATIVE M⁻555 (corresponding to C₁₅H₉I₂O₅S⁻)

### Examples 1-2 to 1-7

### Synthesis of Sulfonium Salt Monomers a-2 to a-7

Sulfonium salt monomers a-2 to a-7 having the following formulas were synthesized using corresponding raw materials and known organic synthesis reactions.

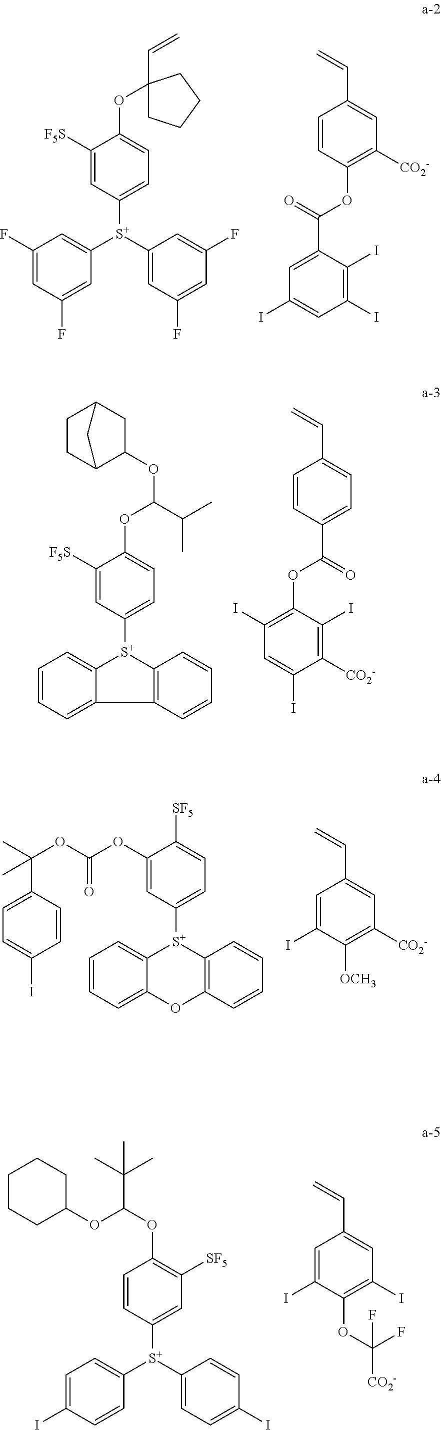

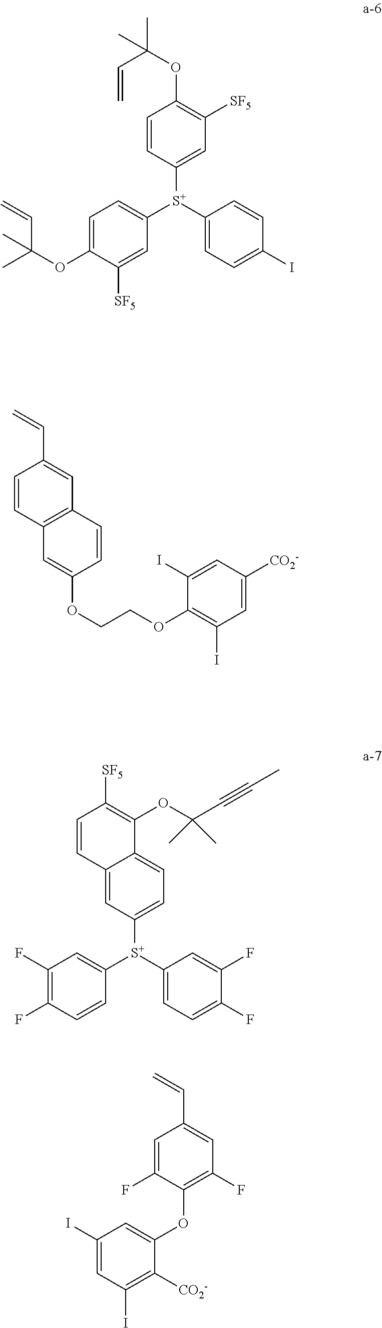

### Comparative Examples 1-1 to 1-5

### Synthesis of Comparative Onium Salt Monomers Ca-1 to Ca-5

Comparative onium salt monomers ca-1 to ca-5 having the following formulas were synthesized using corresponding raw materials and known organic synthesis reactions.

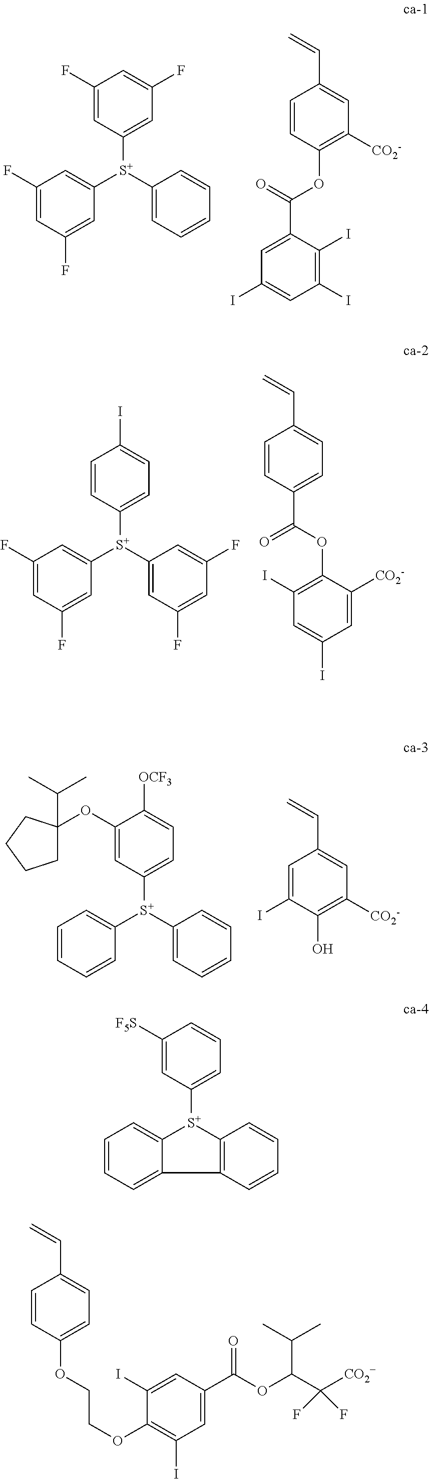

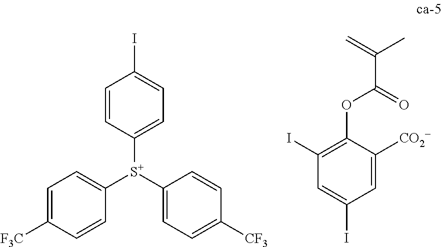

### Synthesis of Base Polymer

Among monomers used for synthesis of a base polymer, monomers other than the monomers a-1 to a-7 and the comparative monomers ca-1 to ca-5 are as follows. 10

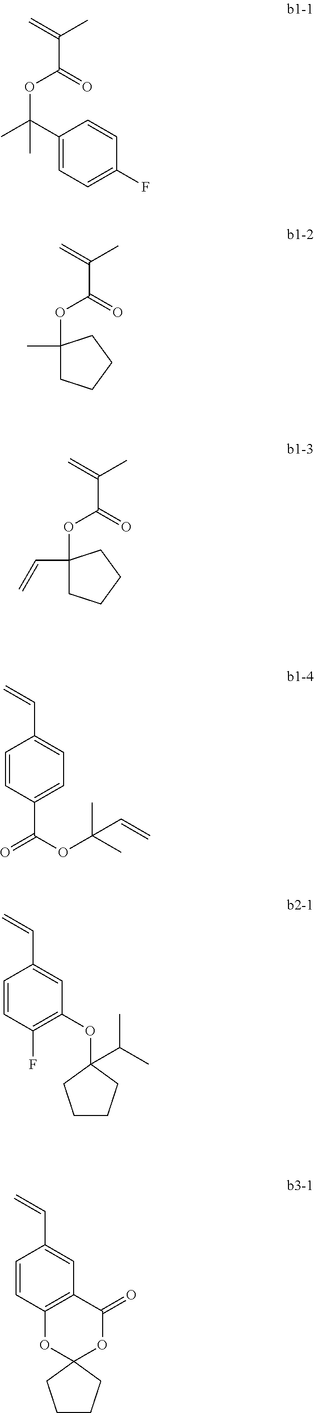

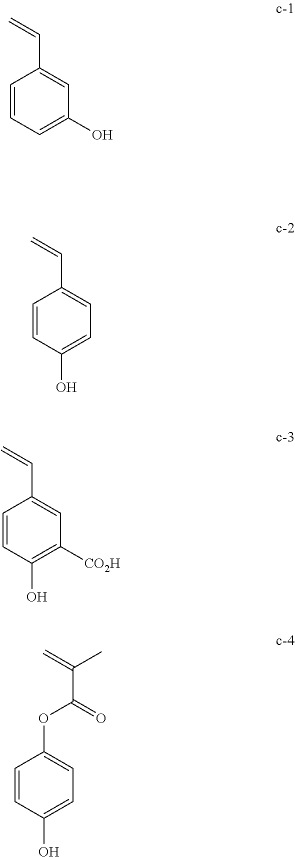

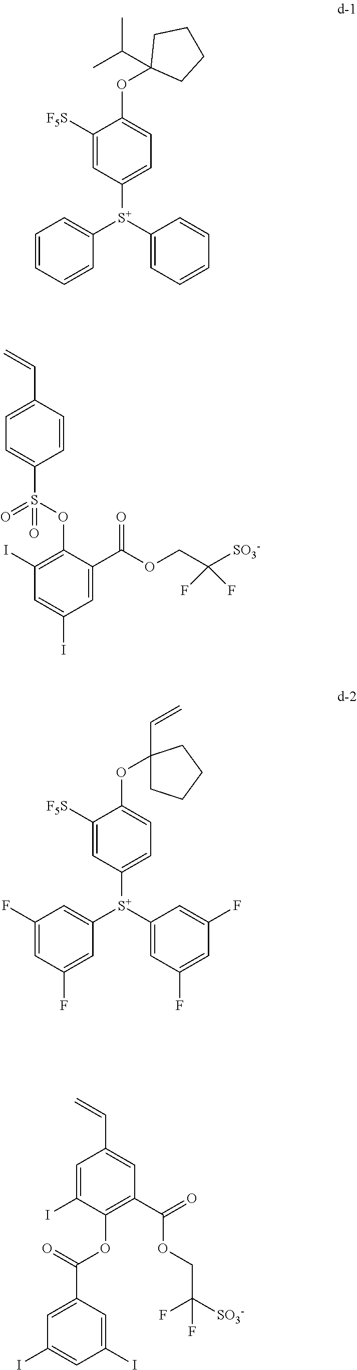

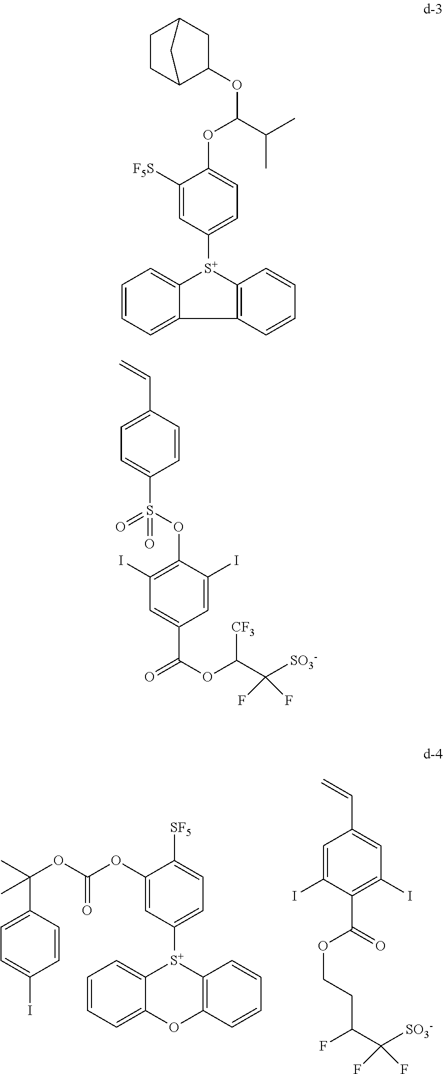

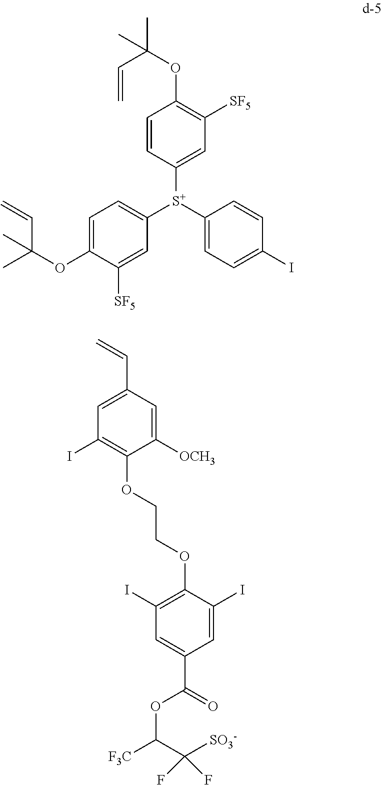

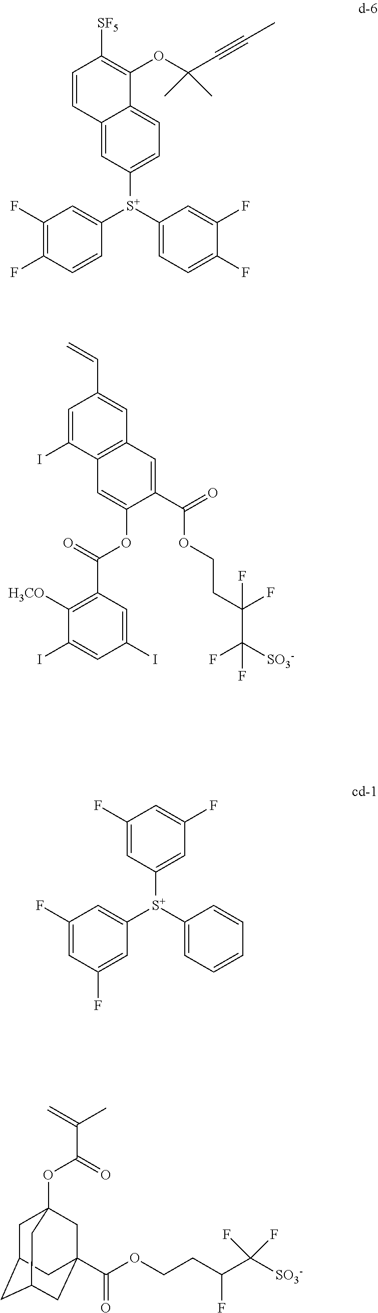

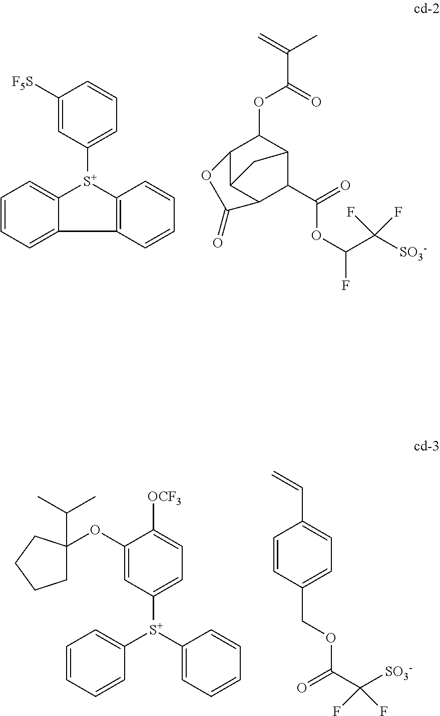

### Example 2-1

### Synthesis of Polymer P-1

In a nitrogen atmosphere, the monomer a-1 (14.0 g), the monomer b1-1 (29.2 g), the monomer c-1 (9.4 g), the monomer d-1 (47.6 g), 3.01 g of V-601 (manufactured by FUJIFILM Wako Pure Chemical Corporation), and 140 g of MEK were put in a flask to prepare a monomer-polymerization initiator solution. In another flask in a nitrogen atmosphere, 46 g of MEK was put, heated to 80° C. while being stirred, and then the monomer-polymerization initiator solution was dropwise added thereto over four hours. After completion of the dropwise addition, stirring was continued for two hours while the temperature of the polymerization liquid was maintained at 80° C., and then the polymerization liquid was cooled to room temperature. The obtained polymerization liquid was dropwise added to 3000 g of vigorously stirred hexane, and a precipitated polymer was separated by filtration. The obtained polymer was washed with 600 g of hexane twice, and then vacuum-dried at 50° C. for 20 hours to obtain a white powder polymer P-1 (amount 97.1 g, yield 97%). The polymer P-1 had Mw of 10100, and Mw/Mn of 1.55. Note that Mw is a value measured in terms of polystyrene by GPC using DMF as a solvent.

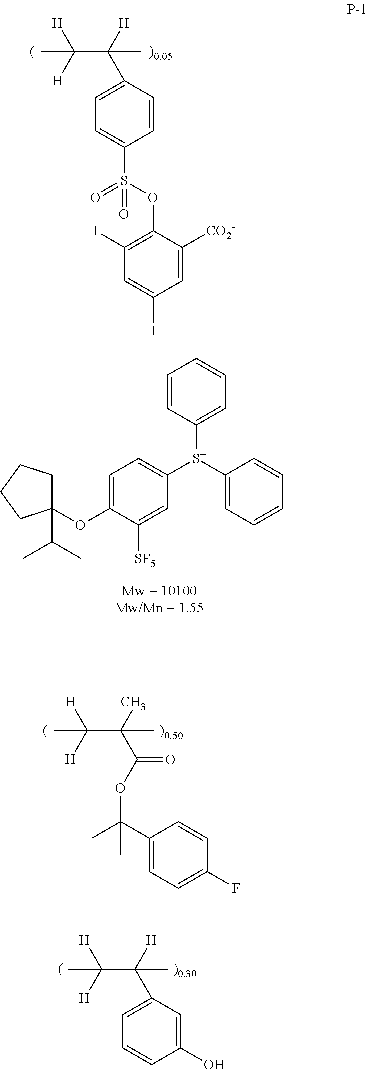

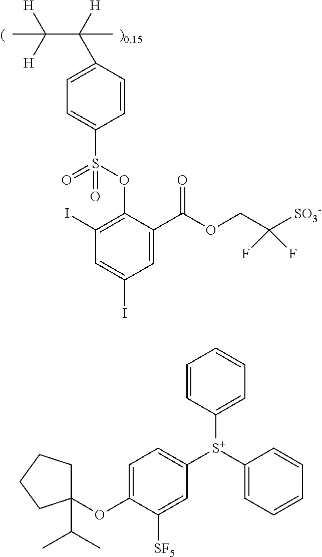

### Examples 2-2 to 2-50 and Comparative Examples 2-1 to 2-38

### Synthesis of Polymers P-2 to P-50 and Comparative Polymers CP-1 to CP-38

Polymers presented in Tables 1 to 3 were manufactured by a similar method to that in Synthesis Example 2-1 except that the type and blending ratio of each monomer were changed.

TABLE 1

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| | | ratio | | ratio | | ratio | | ratio | | ratio | | |
| Polymer | Unit 1 | (mol %) | Unit 2 | (mol %) | Unit 3 | (mol %) | Unit 4 | (mol %) | Unit 5 | (mol %) | Mw | Mw/Mn |
| P-1 | a-1 | 5 | b1-1 | 50 | c-1 | 30 | d-1 | 15 | — | — | 10,100 | 1.55 |
| P-2 | a-2 | 5 | b1-1 | 50 | c-1 | 30 | d-1 | 15 | — | — | 10,200 | 1.54 |
| P-3 | a-3 | 5 | b1-1 | 50 | c-1 | 30 | d-1 | 15 | — | — | 10,400 | 1.52 |
| P-4 | a-4 | 5 | b1-1 | 50 | c-1 | 30 | d-1 | 15 | — | — | 10,200 | 1.53 |
| P-5 | a-5 | 5 | b1-1 | 50 | c-1 | 30 | d-1 | 15 | — | — | 10,400 | 1.54 |
| P-6 | a-6 | 5 | b1-1 | 50 | c-1 | 30 | d-1 | 15 | — | — | 10,200 | 1.54 |
| P-7 | a-7 | 5 | b1-1 | 50 | c-1 | 30 | d-1 | 15 | — | — | 10,300 | 1.54 |
| P-8 | a-1 | 5 | b1-2 | 50 | c-1 | 30 | d-1 | 15 | — | — | 10,300 | 1.55 |
| P-9 | a-2 | 5 | b1-2 | 50 | c-1 | 30 | d-1 | 15 | — | — | 10,200 | 1.52 |
| P-10 | a-3 | 5 | b1-2 | 50 | c-1 | 30 | d-1 | 15 | — | — | 10,100 | 1.53 |
| P-11 | a-4 | 5 | b1-2 | 50 | c-1 | 30 | d-1 | 15 | — | — | 10,500 | 1.53 |
| P-12 | a-5 | 5 | b1-2 | 50 | c-1 | 30 | d-1 | 15 | — | — | 10,100 | 1.54 |
| P-13 | a-6 | 5 | b1-2 | 50 | c-1 | 30 | d-1 | 15 | — | — | 10,200 | 1.56 |
| P-14 | a-7 | 5 | b1-2 | 50 | c-1 | 30 | d-1 | 15 | — | — | 10,300 | 1.54 |
| P-15 | a-1 | 5 | b1-3 | 50 | c-1 | 30 | d-1 | 15 | — | — | 10,000 | 1.53 |
| P-16 | a-2 | 5 | b1-3 | 50 | c-1 | 30 | d-1 | 15 | — | — | 10,200 | 1.53 |
| P-17 | a-3 | 5 | b1-3 | 50 | c-1 | 30 | d-1 | 15 | — | — | 10,100 | 1.52 |
| P-18 | a-4 | 5 | b1-3 | 50 | c-1 | 30 | d-1 | 15 | — | — | 10,400 | 1.53 |
| P-19 | a-5 | 5 | b1-3 | 50 | c-1 | 30 | d-1 | 15 | — | — | 10,200 | 1.54 |
| P-20 | a-6 | 5 | b1-3 | 50 | c-1 | 30 | d-1 | 15 | — | — | 10,300 | 1.55 |
| P-21 | a-7 | 5 | b1-3 | 50 | c-1 | 30 | d-1 | 15 | — | — | 10,300 | 1.54 |
| P-22 | a-1 | 5 | b1-4 | 50 | c-1 | 30 | d-1 | 15 | — | — | 10,400 | 1.54 |
| P-23 | a-2 | 5 | b1-4 | 50 | c-1 | 30 | d-1 | 15 | — | — | 10,200 | 1.52 |
| P-24 | a-3 | 5 | b1-4 | 50 | c-1 | 30 | d-1 | 15 | — | — | 10,300 | 1.53 |
| P-25 | a-4 | 5 | b1-4 | 50 | c-1 | 30 | d-1 | 15 | — | — | 10,200 | 1.54 |
| P-26 | a-5 | 5 | b1-4 | 50 | c-1 | 30 | d-1 | 15 | — | — | 10,200 | 1.54 |
| P-27 | a-6 | 5 | b1-4 | 50 | c-1 | 30 | d-1 | 15 | — | — | 10,300 | 1.56 |
| P-28 | a-7 | 5 | b1-4 | 50 | c-1 | 30 | d-1 | 15 | — | — | 10,400 | 1.53 |
| P-29 | a-1 | 5 | b1-1 | 25 | b2-1 | 25 | c-2 | 30 | d-1 | 15 | 10,300 | 1.54 |
| P-30 | a-1 | 5 | b1-2 | 25 | b2-1 | 25 | c-2 | 30 | d-1 | 15 | 10,500 | 1.52 |

TABLE 2

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| | | ratio | | ratio | | ratio | | ratio | | ratio | | |
| Polymer | Unit 1 | (mol %) | Unit 2 | (mol %) | Unit 3 | (mol %) | Unit 4 | (mol %) | Unit 5 | (mol %) | Mw | Mw/Mn |
| P-31 | a-1 | 5 | b1-3 | 25 | b2-1 | 25 | c-2 | 30 | d-1 | 8 | 10,300 | 1.55 |
| P-32 | a-1 | 5 | b1-3 | 30 | b2-1 | 25 | c-3 | 25 | d-1 | 15 | 10,200 | 1.53 |
| P-33 | a-1 | 5 | b1-1 | 25 | b2-1 | 25 | c-4 | 30 | d-2 | 15 | 10,200 | 1.54 |
| P-34 | a-2 | 5 | b1-1 | 30 | b3-1 | 15 | c-1 | 30 | d-2 | 15 | 10,100 | 1.55 |
| P-35 | a-2 | 5 | b1-1 | 25 | b1-4 | 25 | c-2 | 30 | d-1 | 15 | 10,300 | 1.56 |
| P-36 | a-1 | 5 | b1-1 | 50 | c-1 | 30 | d-2 | 15 | — | — | 10,500 | 1.53 |
| P-37 | a-1 | 5 | b1-1 | 50 | c-1 | 30 | d-3 | 15 | — | — | 10,300 | 1.52 |
| P-38 | a-2 | 5 | b1-1 | 50 | c-1 | 30 | d-4 | 15 | — | — | 10,200 | 1.54 |
| P-39 | a-3 | 5 | b1-1 | 50 | c-1 | 30 | d-5 | 15 | — | — | 10,100 | 1.55 |
| P-40 | a-4 | 5 | b1-1 | 50 | c-1 | 30 | d-6 | 15 | — | — | 10,200 | 1.52 |
| P-41 | a-2 | 5 | b1-1 | 45 | c-2 | 25 | d-1 | 15 | e-1 | 10 | 10,100 | 1.53 |
| P-42 | a-3 | 5 | b1-3 | 45 | c-4 | 25 | d-2 | 15 | e-2 | 10 | 10,000 | 1.54 |
| P-43 | a-4 | 5 | b1-3 | 45 | c-2 | 25 | d-3 | 10 | e-3 | 5 | 10,200 | 1.56 |
| P-44 | a-1 | 7 | b1-1 | 58 | c-1 | 35 | — | — | — | — | 10,100 | 1.53 |
| P-45 | a-2 | 7 | b1-1 | 58 | c-1 | 35 | — | — | — | — | 10,300 | 1.54 |
| P-46 | a-3 | 7 | b1-1 | 58 | c-2 | 35 | — | — | — | — | 10,200 | 1.55 |
| P-47 | a-4 | 7 | b1-3 | 58 | c-3 | 35 | — | — | — | — | 10,400 | 1.52 |
| P-48 | a-5 | 7 | b1-4 | 58 | c-4 | 35 | — | — | — | — | 10,200 | 1.53 |
| P-49 | a-6 | 7 | b1-1 | 29 | b2-1 | 29 | c-1 | 35 | — | — | 10,100 | 1.54 |
| P-50 | a-7 | 7 | b1-1 | 29 | b3-1 | 29 | c-2 | 35 | — | — | 10,000 | 1.53 |

TABLE 3

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| | | ratio | | ratio | | ratio | | ratio | | ratio | | |
| Polymer | Unit 1 | (mol %) | Unit 2 | (mol %) | Unit 3 | (mol %) | Unit 4 | (mol %) | Unit 5 | (mol %) | Mw | Mw/Mn |
| CP-1 | ca-1 | 5 | b1-1 | 50 | c-1 | 30 | d-1 | 15 | — | — | 10,400 | 1.55 |
| CP-2 | ca-2 | 5 | b1-1 | 50 | c-1 | 30 | d-1 | 15 | — | — | 10,200 | 1.55 |
| CP-3 | ca-3 | 5 | b1-1 | 50 | c-1 | 30 | d-1 | 15 | — | — | 10,500 | 1.54 |
| CP-4 | ca-4 | 5 | b1-1 | 50 | c-1 | 30 | d-1 | 15 | — | — | 10,200 | 1.52 |
| CP-5 | ca-5 | 5 | b1-1 | 50 | c-1 | 30 | d-1 | 15 | — | — | 10,400 | 1.53 |
| CP-6 | ca-1 | 5 | b1-2 | 50 | c-1 | 30 | d-1 | 15 | — | — | 10,500 | 1.54 |
| CP-7 | ca-2 | 5 | b1-2 | 50 | c-1 | 30 | d-1 | 15 | — | — | 10,200 | 1.54 |
| CP-8 | ca-3 | 5 | b1-2 | 50 | c-1 | 30 | d-1 | 15 | — | — | 10,600 | 1.56 |
| CP-9 | ca-4 | 5 | b1-2 | 50 | c-1 | 30 | d-1 | 15 | — | — | 10,500 | 1.53 |
| CP-10 | ca-5 | 5 | b1-2 | 50 | c-1 | 30 | d-1 | 15 | — | — | 10,200 | 1.54 |
| CP-11 | ca-1 | 5 | b1-3 | 50 | c-1 | 30 | d-1 | 15 | — | — | 10,400 | 1.52 |
| CP-12 | ca-2 | 5 | b1-3 | 50 | c-1 | 30 | d-1 | 15 | — | — | 10,400 | 1.53 |
| CP-13 | ca-3 | 5 | b1-3 | 50 | c-1 | 30 | d-1 | 15 | — | — | 10,600 | 1.54 |
| CP-14 | ca-4 | 5 | b1-3 | 50 | c-1 | 30 | d-1 | 15 | — | — | 10,600 | 1.55 |
| CP-15 | ca-5 | 5 | b1-3 | 50 | c-1 | 30 | d-1 | 15 | — | — | 10,200 | 1.54 |
| CP-16 | ca-1 | 5 | b1-4 | 50 | c-1 | 30 | d-1 | 15 | — | — | 10,300 | 1.53 |
| CP-17 | ca-2 | 5 | b1-4 | 50 | c-1 | 30 | d-1 | 15 | — | — | 10,300 | 1.56 |
| CP-18 | ca-3 | 5 | b1-4 | 50 | c-1 | 30 | d-1 | 15 | — | — | 10,500 | 1.53 |
| CP-19 | ca-4 | 5 | b1-4 | 50 | c-1 | 30 | d-1 | 15 | — | — | 10,600 | 1.52 |
| CP-20 | ca-5 | 5 | b1-4 | 50 | c-1 | 30 | d-1 | 15 | — | — | 10,400 | 1.54 |
| CP-21 | ca-1 | 5 | b1-1 | 25 | b2-1 | 25 | c-2 | 30 | d-1 | 15 | 10,200 | 1.53 |
| CP-22 | ca-2 | 5 | b1-2 | 25 | b2-1 | 25 | c-2 | 30 | d-2 | 15 | 10,200 | 1.54 |
| CP-23 | ca-4 | 5 | b1-3 | 25 | b2-1 | 25 | c-2 | 30 | d-4 | 8 | 10,100 | 1.54 |
| CP-24 | ca-1 | 5 | b1-1 | 25 | b2-1 | 25 | c-2 | 30 | cd-1 | 15 | 10,200 | 1.53 |
| CP-25 | ca-2 | 5 | b1-2 | 25 | b2-1 | 25 | c-2 | 30 | cd-2 | 15 | 10,700 | 1.54 |
| CP-26 | ca-5 | 5 | b1-3 | 25 | b2-1 | 25 | c-2 | 30 | cd-3 | 15 | 10,300 | 1.55 |
| CP-27 | ca-1 | 5 | b1-1 | 50 | c-1 | 30 | cd-1 | 15 | — | — | 10,300 | 1.52 |
| CP-28 | ca-1 | 5 | b1-1 | 50 | c-1 | 30 | cd-2 | 15 | — | — | 10,200 | 1.55 |
| CP-29 | ca-2 | 5 | b1-1 | 50 | c-1 | 30 | cd-3 | 15 | — | — | 10,200 | 1.56 |
| CP-30 | ca-1 | 5 | b1-1 | 45 | c-2 | 25 | d-1 | 15 | e-1 | 10 | 10,300 | 1.52 |
| CP-31 | ca-2 | 5 | b1-3 | 45 | c-4 | 25 | cd-1 | 15 | e-2 | 10 | 10,500 | 1.55 |
| CP-32 | ca-4 | 5 | b1-3 | 45 | c-2 | 25 | cd-2 | 10 | e-3 | 5 | 10,600 | 1.53 |
| CP-33 | ca-1 | 7 | b1-1 | 58 | c-1 | 35 | — | — | — | — | 10,200 | 1.53 |
| CP-34 | ca-2 | 7 | b1-1 | 58 | c-2 | 35 | — | — | — | — | 10,400 | 1.54 |
| CP-35 | ca-4 | 7 | b1-2 | 58 | c-2 | 35 | — | — | — | — | 10,200 | 1.52 |
| CP-36 | ca-1 | 7 | b1-1 | 29 | b2-1 | 29 | c-1 | 35 | — | — | 10,600 | 1.55 |
| CP-37 | b1-1 | 55 | c-1 | 30 | cd-1 | 15 | — | — | — | — | 10,400 | 1.53 |
| CP-38 | b1-2 | 50 | c-2 | 50 | — | — | — | — | — | — | 10,200 | 1.53 |

### Preparation of Chemically Amplified Resist Composition

### Examples 3-1 to 3-53 and Comparative Examples 3-1 to 3-40

Predetermined components selected from the base polymer of the present invention (P-1 to P-50), the base polymer for comparison (CP-1 to CP-38), the acid generator (PAG-1 and PAG-2), and the quencher (SQ-1 and AQ-1) were dissolved in a solvent containing 0.01 wt % of FC-4430 manufactured by 3M as a surfactant with a composition presented in Tables 4 to 6 below to prepare a solution, and the solution was filtered through a 0.2 μm Teflon® filter to prepare a chemically amplified resist composition (R-1 to R-53 and CR-1 to CR-40).

TABLE 4

| | | Base | | Acid | | | |
|---|---|---|---|---|---|---|---|
| | Resist | polymer | Quencher | generator | Solvent 1 | Solvent 2 | Solvent 3 |
| Example | composition | (pbw) | (pbw) | (pbw) | (pbw) | (pbw) | (pbw) |
| 3-1 | R-1 | P-1 (80) | — | — | PGMEA (2250) | EL (2800) | DAA (550) |
| 3-2 | R-2 | P-2 (80) | — | — | PGMEA (2250) | EL (2800) | DAA (550) |
| 3-3 | R-3 | P-3 (80) | — | — | PGMEA (2250) | EL (2800) | DAA (550) |
| 3-4 | R-4 | P-4 (80) | — | — | PGMEA (2250) | EL (2800) | DAA (550) |
| 3-5 | R-5 | P-5 (80) | — | — | PGMEA (2250) | EL (2800) | DAA (550) |
| 3-6 | R-6 | P-6 (80) | — | — | PGMEA (2250) | EL (2800) | DAA (550) |
| 3-7 | R-7 | P-7 (80) | — | — | PGMEA (2250) | EL (2800) | DAA (550) |
| 3-8 | R-8 | P-8 (80) | — | — | PGMEA (2250) | EL (2800) | DAA (550) |
| 3-9 | R-9 | P-9 (80) | — | — | PGMEA (2250) | EL (2800) | DAA (550) |
| 3-10 | R-10 | P-10 (80) | — | — | PGMEA (2250) | EL (2800) | DAA (550) |
| 3-11 | R-11 | P-11 (80) | — | — | PGMEA (2250) | EL (2800) | DAA (550) |
| 3-12 | R-12 | P-12 (80) | — | — | PGMEA (2250) | EL (2800) | DAA (550) |
| 3-13 | R-13 | P-13 (80) | — | — | PGMEA (2250) | EL (2800) | DAA (550) |
| 3-14 | R-14 | P-14 (80) | — | — | PGMEA (2250) | EL (2800) | DAA (550) |
| 3-15 | R-15 | P-15 (80) | — | — | PGMEA (2250) | EL (2800) | DAA (550) |
| 3-16 | R-16 | P-16 (80) | — | — | PGMEA (2250) | EL (2800) | DAA (550) |
| 3-17 | R-17 | P-17 (80) | — | — | PGMEA (2250) | EL (2800) | DAA (550) |
| 3-18 | R-18 | P-18 (80) | — | — | PGMEA (2250) | EL (2800) | DAA (550) |
| 3-19 | R-19 | P-19 (80) | — | — | PGMEA (2250) | EL (2800) | DAA (550) |
| 3-20 | R-20 | P-20 (80) | — | — | PGMEA (2250) | EL (2800) | DAA (550) |
| 3-21 | R-21 | P-21 (80) | — | — | PGMEA (2250) | EL (2800) | DAA (550) |
| 3-22 | R-22 | P-22 (80) | — | — | PGMEA (2250) | EL (2800) | DAA (550) |
| 3-23 | R-23 | P-23 (80) | — | — | PGMEA (2250) | EL (2800) | DAA (550) |
| 3-24 | R-24 | P-24 (80) | — | — | PGMEA (2250) | EL (2800) | DAA (550) |
| 3-25 | R-25 | P-25 (80) | — | — | PGMEA (2250) | EL (2800) | DAA (550) |
| 3-26 | R-26 | P-26 (80) | — | — | PGMEA (2250) | EL (2800) | DAA (550) |
| 3-27 | R-27 | P-27 (80) | — | — | PGMEA (2250) | EL (2800) | DAA (550) |
| 3-28 | R-28 | P-28 (80) | — | — | PGMEA (2250) | EL (2800) | DAA (550) |
| 3-29 | R-29 | P-29 (80) | — | — | PGMEA (2250) | EL (2800) | DAA (550) |
| 3-30 | R-30 | P-30 (80) | — | — | PGMEA (2250) | EL (2800) | DAA (550) |

TABLE 5

| | | Base | | Acid | | | |
|---|---|---|---|---|---|---|---|
| | Resist | polymer | Quencher | generator | Solvent 1 | Solvent 2 | Solvent 3 |
| Example | composition | (pbw) | (pbw) | (pbw) | (pbw) | (pbw) | (pbw) |
| 3-31 | R-31 | P-31 (80) | — | PAG-1 (10) | PGMEA (2250) | EL (2800) | DAA (550) |
| 3-32 | R-32 | P-32 (80) | — | — | PGMEA (2250) | EL (2800) | DAA (550) |
| 3-33 | R-33 | P-33 (80) | — | — | PGMEA (2250) | EL (2800) | DAA (550) |
| 3-34 | R-34 | P-34 (80) | — | — | PGMEA (2250) | EL (2800) | DAA (550) |
| 3-35 | R-35 | P-35 (80) | — | — | PGMEA (2250) | EL (2800) | DAA (550) |
| 3-36 | R-36 | P-36 (80) | — | — | PGMEA (2250) | EL (2800) | DAA (550) |
| 3-37 | R-37 | P-37 (80) | — | — | PGMEA (2250) | EL (2800) | DAA (550) |
| 3-38 | R-38 | P-38 (80) | — | — | PGMEA (2250) | EL (2800) | DAA (550) |
| 3-39 | R-39 | P-39 (80) | — | — | PGMEA (2250) | EL (2800) | DAA (550) |
| 3-40 | R-40 | P-40 (80) | — | — | PGMEA (2250) | EL (2800) | DAA (550) |
| 3-41 | R-41 | P-41 (80) | — | — | PGMEA (2250) | EL (2800) | DAA (550) |
| 3-42 | R-42 | P-42 (80) | — | — | PGMEA (2250) | EL (2800) | DAA (550) |
| 3-43 | R-43 | P-43 (80) | — | PAG-2 (10) | PGMEA (2250) | EL (2800) | DAA (550) |
| 3-44 | R-44 | P-44 (80) | — | PAG-1 (20) | PGMEA (2250) | EL (2800) | DAA (550) |
| 3-45 | R-45 | P-45 (80) | — | PAG-1 (20) | PGMEA (2250) | EL (2800) | DAA (550) |
| 3-46 | R-46 | P-46 (80) | — | PAG-1 (15) | PGMEA (2250) | EL (2800) | DAA (550) |
| | | | | PAG-3 (5) | | | |
| 3-47 | R-47 | P-47 (80) | — | PAG-2 (20) | PGMEA (2250) | EL (2800) | DAA (550) |
| 3-48 | R-48 | P-48 (80) | — | PAG-1 (20) | PGMEA (2250) | EL (2800) | DAA (550) |
| 3-49 | R-49 | P-49 (80) | — | PAG-2 (15) | PGMEA (2250) | EL (2800) | DAA (550) |
| | | | | PAG-3 (5) | | | |
| 3-50 | R-50 | P-50 (80) | — | PAG-1 (20) | PGMEA (2250) | EL (2800) | DAA (550) |
| 3-51 | R-51 | P-1 (80) | SQ-1 (4) | — | PGMEA (2250) | EL (2800) | DAA (550) |
| 3-52 | R-52 | P-1 (80) | AQ-1 (4) | — | PGMEA (2250) | EL (2800) | DAA (550) |
| 3-53 | R-53 | P-1 (80) | SQ-1 (2) | — | PGMEA (2250) | EL (2800) | DAA (550) |
| | | | AQ-1 (2) | | | | |

TABLE 6

| | Resist | Base | | Acid | | | |
|---|---|---|---|---|---|---|---|
| Comparative | com- | polymer | Quencher | generator | Solvent 1 | Solvent 2 | Solvent 3 |
| Example | position | (pbw) | (pbw) | (pbw) | (pbw) | (pbw) | (pbw) |
| 3-1 | CR-1 | CP-1(80) | — | — | PGMEA (2250) | EL (2800) | DAA (550) |
| 3-2 | CR-2 | CP-2 (80) | — | — | PGMEA (2250) | EL (2800) | DAA (550) |
| 3-3 | CR-3 | CP-3 (80) | — | — | PGMEA (2250) | EL (2800) | DAA (550) |
| 3-4 | CR-4 | CP-4 (80) | — | — | PGMEA (2250) | EL (2800) | DAA (550) |
| 3-5 | CR-5 | CP-5 (80) | — | — | PGMEA (2250) | EL (2800) | DAA (550) |
| 3-6 | CR-6 | CP-6 (80) | — | — | PGMEA (2250) | EL (2800) | DAA (550) |
| 3-7 | CR-7 | CP-7(80) | — | — | PGMEA (2250) | EL (2800) | DAA (550) |
| 3-8 | CR-8 | CP-8 (80) | — | — | PGMEA (2250) | EL (2800) | DAA (550) |
| 3-9 | CR-9 | CP-9 (80) | — | — | PGMEA (2250) | EL (2800) | DAA (550) |
| 3-10 | CR-10 | CP-10 (80) | — | — | PGMEA (2250) | EL (2800) | DAA (550) |
| 3-11 | CR-11 | CP-11 (80) | — | — | PGMEA (2250) | EL (2800) | DAA (550) |
| 3-12 | CR-12 | CP-12 (80) | — | — | PGMEA (2250) | EL (2800) | DAA (550) |
| 3-13 | CR-13 | CP-13 (80) | — | — | PGMEA (2250) | EL (2800) | DAA (550) |
| 3-14 | CR-14 | CP-14 (80) | — | — | PGMEA (2250) | EL (2800) | DAA (550) |
| 3-15 | CR-15 | CP-15 (80) | — | — | PGMEA (2250) | EL (2800) | DAA (550) |
| 3-16 | CR-16 | CP-16 (80) | — | — | PGMEA (2250) | EL (2800) | DAA (550) |
| 3-17 | CR-17 | CP-17(80) | — | — | PGMEA (2250) | EL (2800) | DAA (550) |
| 3-18 | CR-18 | CP-18 (80) | — | — | PGMEA (2250) | EL (2800) | DAA (550) |
| 3-19 | CR-19 | CP-19 (80) | — | — | PGMEA (2250) | EL (2800) | DAA (550) |
| 3-20 | CR-20 | CP-20 (80) | — | — | PGMEA (2250) | EL (2800) | DAA (550) |
| 3-21 | CR-21 | CP-21 (80) | — | — | PGMEA (2250) | EL (2800) | DAA (550) |
| 3-22 | CR-22 | CP-22 (80) | — | — | PGMEA (2250) | EL (2800) | DAA (550) |
| 3-23 | CR-23 | CP-23 (80) | — | PAG-1 (10) | PGMEA (2250) | EL (2800) | DAA (550) |
| 3-24 | CR-24 | CP-24 (80) | — | — | PGMEA (2250) | EL (2800) | DAA (550) |
| 3-25 | CR-25 | CP-25 (80) | — | — | PGMEA (2250) | EL (2800) | DAA (550) |
| 3-26 | CR-26 | CP-26 (80) | — | — | PGMEA (2250) | EL (2800) | DAA (550) |
| 3-27 | CR-27 | CP-27 (80) | — | — | PGMEA (2250) | EL (2800) | DAA (550) |
| 3-28 | CR-28 | CP-28 (80) | — | — | PGMEA (2250) | EL (2800) | DAA (550) |
| 3-29 | CR-29 | CP-29 (80) | — | — | PGMEA (2250) | EL (2800) | DAA (550) |
| 3-30 | CR-30 | CP-30 (80) | — | — | PGMEA (2250) | EL (2800) | DAA (550) |
| 3-31 | CR-31 | CP-31 (80) | — | — | PGMEA (2250) | EL (2800) | DAA (550) |
| 3-32 | CR-32 | CP-32 (80) | — | PAG-2 (10) | PGMEA (2250) | EL (2800) | DAA (550) |
| 3-33 | CR-33 | CP-33 (80) | — | PAG-1 (20) | PGMEA (2250) | EL (2800) | DAA (550) |
| 3-34 | CR-34 | CP-34 (80) | — | PAG-1 (15) | PGMEA (2250) | EL (2800) | DAA (550) |
| | | | | PAG-3 (5) | | | |
| 3-35 | CR-35 | CP-35 (80) | — | PAG-2 (15) | PGMEA (2250) | EL (2800) | DAA (550) |
| | | | | PAG-3 (5) | | | |
| 3-36 | CR-36 | CP-36 (80) | — | PAG-1 (20) | PGMEA (2250) | EL (2800) | DAA (550) |
| 3-37 | CR-37 | CP-37 (80) | SQ-1(8) | — | PGMEA (2250) | EL (2800) | DAA (550) |
| 3-38 | CR-38 | CP-38 (80) | SQ-1 (8) | PAG-1 (20) | PGMEA (2250) | EL (2800) | DAA (550) |
| 3-39 | CR-39 | CP-38 (80) | AQ-1 (8) | PAG-2 (20) | PGMEA (2250) | EL (2800) | DAA (550) |
| 3-40 | CR-40 | CP-38 (80) | SQ-1 (4) | PAG-1 (15) | PGMEA (2250) | EL (2800) | DAA (550) |
| | | | AQ-1 (4) | PAG-3 (5) | | | |

In Tables 4 to 6, the solvent, the quencher (SQ-1 and AQ-1), and the acid generator (PAG-1, PAG-2, and PAG-3) are as follows.

### Solvent:

PGMEA (propylene glycol monomethyl ether acetate)

EL (ethyl lactate)

DAA (diacetone alcohol)

### Quenchers: SQ-1 and AQ-1

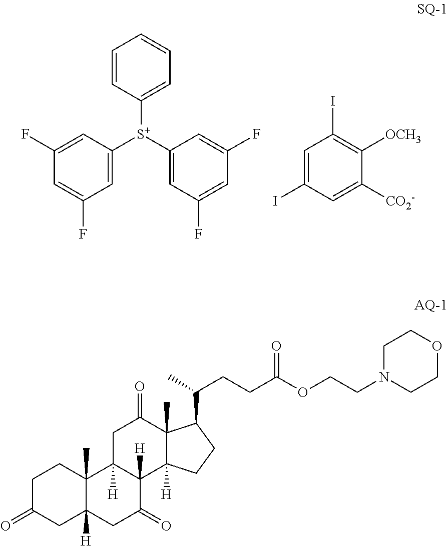

Acid generators: PAG-1, PAG-2, and PAG-3

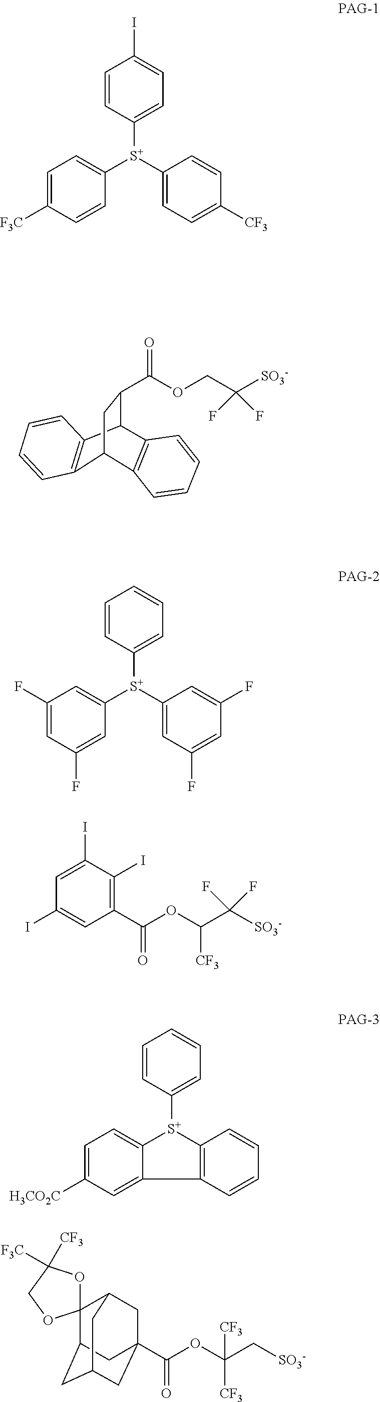

### [4] EUV Lithography Evaluation (1)

### Examples 4-1 to 4-53 and Comparative Examples 4-1 to 4-40

Each of the chemically amplified resist compositions (R-1 to R-53 and CR-1 to CR-40) presented in Tables 4 to 6 was spin-coated on a Si substrate formed with a silicon-containing spin-on hard mask SHB-A940 (silicon content: 43 wt %) manufactured by Shin-Etsu Chemical Co., Ltd. in a film thickness of 20 nm, and prebaked at 100° C. for 60 seconds using a hot plate to prepare a resist film having a film thickness of 50 nm. The resist film was exposed to light so as to provide an LS pattern having an on-wafer dimension of 18 nm and a pitch of 36 nm with an EUV scanner NXE3300 (NA: 0.33, σ: 0.9/0.6, dipole illumination) manufactured by ASML while an exposure dose and a focus were changed (exposure dose pitch: 1 mJ/cm², focus pitch: 0.020 μm), and after the exposure, PEB was performed at temperatures presented in Tables 7 to 9 for 60 seconds. Thereafter, puddle development was performed with a 2.38 wt % TMAH aqueous solution for 30 seconds, rinsing was performed with a surfactant-containing rinse material, and spin-drying was performed to obtain a positive pattern.

The obtained LS pattern was observed with a length measuring SEM (CG6300) manufactured by Hitachi High-Tech Corporation, and sensitivity, EL, LWR, DOF, and a collapse limit were evaluated according to the following methods. In addition, development defect evaluation of the obtained LS pattern was performed. Results thereof are presented in Tables 7 to 9.

### [Sensitivity Evaluation]

An optimum exposure dose Eop (mJ/cm²) at which an LS pattern having a line width of 18 nm and a pitch of 36 nm could be obtained was determined and taken as sensitivity. The smaller this value is, the higher the sensitivity is.

### [EL Evaluation]

EL (unit: %) was determined from an exposure dose formed within ±10% (16.2 to 19.8 nm) of the space width of 18 nm in the LS pattern by the following formula. The larger this value is, the better the performance is.

$$EL ( \% ) = ( \vert {E}_{1} - {E}_{2} \vert / Eop ) \times 100$$

E₁: Optimum exposure dose that provides an LS pattern having a line width of 16.2 nm and a pitch of 36 nm
E₂: Optimum exposure dose that provides an LS pattern having a line width of 19.8 nm and a pitch of 36 nm
Eop: Optimum exposure dose that provides an LS pattern having a line width of 18 nm and a pitch of 36 nm

### [LWR Evaluation]

In the LS pattern obtained by irradiation with Eop, the dimensions of ten points in a longitudinal direction of the line were measured, and from the result, a value (3σ) three times a standard deviation (σ) was obtained as LWR. As this value is smaller, a pattern having a smaller roughness and a more uniform line width can be obtained.

### [DOF Evaluation]

As focus depth evaluation, a focus range formed in a range of ±10% (16.2 to 19.8 nm) of a dimension of 18 nm in the LS pattern was obtained. The larger this value is, the wider focus depth is.

### [Evaluation of Line Pattern Collapse Limit]

The line dimensions of ten points in a longitudinal direction were measured at each exposure dose in an optimal focus of the LS pattern. The thinnest line dimension obtained without collapse was defined as a collapse limit dimension. The smaller this value is, the better a collapse limit is.

### [Development Defect Evaluation]

In the LS pattern with a line width of 18 nm and a pitch of 36 nm formed with the optimum exposure dose, by using a defect inspection apparatus KLA2360 (trade name) manufactured by KLA Corporation and setting a pixel size of the defect inspection apparatus to 0.16 μm and a threshold thereof to 20, the number of defects (number/cm²) extracted from a difference caused by superimposition on a comparative image in units of pixels was detected, and the number of defects (number/cm²) per unit area was calculated. Thereafter, development defects were classified and extracted from all defects by defect review, and the number of development defects (defects/cm²) per unit area was calculated. A sample is rated “A” for a count of less than 0.5, “B” for a count of 0.5 to less than 1.0, “C” for a count of 1.0 to less than 5.0, and “D” for a count of 5.0 or more. A smaller value indicates better performance.

TABLE 7

| | | | Optimum | | | | | |
|---|---|---|---|---|---|---|---|---|
| Ex- | Resist | PEB | exposure | | | | Collapse | Develop- |
| am- | com- | temp. | dose | EL | LWR | DOF | limit | ment |
| ple | position | (° C.) | (mJ/cm²) | (%) | (nm) | (nm) | (nm) | defects |
| 4-1 | R-1 | 95 | 32 | 19 | 2.2 | 110 | 11.0 | A |
| 4-2 | R-2 | 100 | 32 | 18 | 2.3 | 120 | 10.8 | A |
| 4-3 | R-3 | 100 | 31 | 17 | 2.2 | 100 | 10.9 | A |
| 4-4 | R-4 | 95 | 32 | 18 | 2.4 | 120 | 11.2 | A |
| 4-5 | R-5 | 105 | 33 | 17 | 2.4 | 110 | 11.1 | A |
| 4-6 | R-6 | 100 | 34 | 17 | 2.3 | 110 | 10.9 | A |
| 4-7 | R-7 | 95 | 33 | 18 | 2.1 | 110 | 11.1 | A |
| 4-8 | R-8 | 95 | 32 | 17 | 2.4 | 100 | 11.2 | A |
| 4-9 | R-9 | 100 | 31 | 19 | 2.2 | 120 | 11.0 | A |
| 4-10 | R-10 | 100 | 33 | 16 | 2.2 | 110 | 11.1 | A |
| 4-11 | R-11 | 100 | 33 | 18 | 2.3 | 100 | 10.8 | A |
| 4-12 | R-12 | 95 | 34 | 18 | 2.3 | 110 | 11.2 | A |
| 4-13 | R-13 | 105 | 32 | 19 | 2.5 | 100 | 11.3 | A |
| 4-14 | R-14 | 100 | 32 | 17 | 2.3 | 110 | 11.2 | A |
| 4-15 | R-15 | 95 | 33 | 18 | 2.3 | 120 | 11.2 | A |
| 4-16 | R-16 | 95 | 32 | 18 | 2.4 | 110 | 10.9 | A |
| 4-17 | R-17 | 100 | 33 | 19 | 2.4 | 110 | 10.8 | A |
| 4-18 | R-18 | 95 | 33 | 16 | 2.2 | 110 | 11.0 | A |
| 4-19 | R-19 | 95 | 31 | 17 | 2.4 | 120 | 11.3 | A |
| 4-20 | R-20 | 100 | 32 | 19 | 2.3 | 120 | 11.2 | A |
| 4-21 | R-21 | 100 | 33 | 18 | 2.3 | 110 | 11.0 | A |
| 4-22 | R-22 | 95 | 34 | 17 | 2.3 | 110 | 11.1 | A |
| 4-23 | R-23 | 100 | 32 | 17 | 2.2 | 120 | 11.2 | A |
| 4-24 | R-24 | 95 | 35 | 18 | 2.2 | 110 | 11.1 | A |
| 4-25 | R-25 | 100 | 32 | 19 | 2.4 | 110 | 11.2 | A |
| 4-26 | R-26 | 100 | 32 | 17 | 2.3 | 120 | 11.0 | A |
| 4-27 | R-27 | 95 | 32 | 18 | 2.5 | 120 | 11.2 | A |
| 4-28 | R-28 | 100 | 32 | 18 | 2.4 | 110 | 11.1 | A |
| 4-29 | R-29 | 100 | 34 | 18 | 2.2 | 120 | 11.2 | A |
| 4-30 | R-30 | 95 | 33 | 17 | 2.3 | 110 | 11.2 | A |

TABLE 8

| | | | Optimum | | | | | |
|---|---|---|---|---|---|---|---|---|
| Ex- | Resist | PEB | exposure | | | | Collapse | Develop- |
| am- | com- | temp. | dose | EL | LWR | DOF | limit | ment |
| ple | position | (° C.) | (mJ/cm²) | (%) | (nm) | (nm) | (nm) | defects |
| 4-31 | R-31 | 100 | 32 | 19 | 2.4 | 120 | 11.2 | A |
| 4-32 | R-32 | 100 | 32 | 18 | 2.3 | 120 | 11.1 | A |
| 4-33 | R-33 | 100 | 33 | 17 | 2.2 | 110 | 10.9 | A |
| 4-34 | R-34 | 95 | 35 | 19 | 2.3 | 120 | 11.2 | A |
| 4-35 | R-35 | 100 | 32 | 17 | 2.2 | 100 | 11.3 | A |
| 4-36 | R-36 | 95 | 31 | 18 | 2.4 | 120 | 11.1 | A |
| 4-37 | R-37 | 100 | 33 | 18 | 2.2 | 110 | 11.1 | A |
| 4-38 | R-38 | 100 | 33 | 17 | 2.1 | 120 | 11.0 | A |
| 4-39 | R-39 | 95 | 34 | 18 | 2.3 | 120 | 11.4 | A |
| 4-40 | R-40 | 100 | 32 | 16 | 2.3 | 100 | 11.2 | A |
| 4-41 | R-41 | 95 | 33 | 16 | 2.2 | 100 | 11.1 | A |
| 4-42 | R-42 | 95 | 33 | 18 | 2.2 | 110 | 11.3 | A |
| 4-43 | R-43 | 100 | 32 | 19 | 2.3 | 120 | 10.7 | A |
| 4-44 | R-44 | 100 | 33 | 17 | 2.3 | 110 | 11.1 | A |
| 4-45 | R-45 | 95 | 33 | 18 | 2.2 | 100 | 11.2 | A |
| 4-46 | R-46 | 95 | 32 | 18 | 2.1 | 110 | 11.1 | A |
| 4-47 | R-47 | 100 | 34 | 17 | 2.3 | 120 | 10.9 | A |
| 4-48 | R-48 | 95 | 32 | 19 | 2.2 | 110 | 11.1 | A |
| 4-49 | R-49 | 95 | 31 | 18 | 2.3 | 100 | 10.7 | A |
| 4-50 | R-50 | 100 | 33 | 18 | 2.4 | 100 | 11.3 | A |
| 4-51 | R-51 | 95 | 33 | 18 | 2.2 | 110 | 11.2 | A |
| 4-52 | R-52 | 95 | 34 | 17 | 2.3 | 120 | 11.0 | A |
| 4-53 | R-53 | 100 | 33 | 18 | 2.4 | 110 | 11.1 | A |

TABLE 9

| Compar- | | | Optimum | | | | Col- | |
|---|---|---|---|---|---|---|---|---|
| ative | Resist | PEB | exposure | | | | lapse | Develop- |
| Exam- | com- | temp. | dose | EL | LWR | DOF | limit | ment |
| ple | position | (° C.) | (mJ/cm²) | (%) | (nm) | (nm) | (nm) | defects |
| 4-1 | CR-1 | 100 | 38 | 13 | 3 | 90 | 12.5 | B |
| 4-2 | CR-2 | 95 | 39 | 12 | 3.3 | 90 | 12.3 | C |
| 4-3 | CR-3 | 100 | 37 | 14 | 3.2 | 80 | 12.3 | C |
| 4-4 | CR-4 | 105 | 37 | 14 | 3.1 | 90 | 12.4 | C |
| 4-5 | CR-5 | 100 | 37 | 14 | 3.0 | 80 | 12.2 | B |
| 4-6 | CR-6 | 95 | 42 | 12 | 2.8 | 90 | 12.5 | C |
| 4-7 | CR-7 | 100 | 39 | 12 | 3.0 | 90 | 13.2 | B |
| 4-8 | CR-8 | 105 | 39 | 14 | 3.1 | 90 | 12.9 | C |
| 4-9 | CR-9 | 95 | 41 | 14 | 3.1 | 80 | 13.3 | B |
| 4-10 | CR-10 | 100 | 39 | 12 | 2.9 | 90 | 13.2 | C |
| 4-11 | CR-11 | 95 | 39 | 13 | 3.1 | 90 | 12.7 | B |
| 4-12 | CR-12 | 100 | 37 | 14 | 3.1 | 90 | 12.8 | B |
| 4-13 | CR-13 | 100 | 40 | 12 | 3.1 | 90 | 13.2 | C |
| 4-14 | CR-14 | 95 | 38 | 13 | 3.0 | 90 | 12.6 | B |
| 4-15 | CR-15 | 105 | 40 | 12 | 3.2 | 90 | 12.5 | C |
| 4-16 | CR-16 | 100 | 38 | 12 | 2.9 | 90 | 12.4 | C |
| 4-17 | CR-17 | 100 | 38 | 11 | 3.1 | 80 | 13.0 | C |
| 4-18 | CR-18 | 95 | 38 | 13 | 2.8 | 80 | 13.4 | C |
| 4-19 | CR-19 | 100 | 39 | 12 | 3.0 | 90 | 12.5 | B |
| 4-20 | CR-20 | 95 | 37 | 14 | 3.1 | 80 | 13.5 | C |
| 4-21 | CR-21 | 100 | 40 | 13 | 2.8 | 90 | 12.8 | B |
| 4-22 | CR-22 | 100 | 40 | 15 | 2.7 | 90 | 13.2 | C |
| 4-23 | CR-23 | 100 | 39 | 13 | 3.1 | 80 | 12.6 | C |
| 4-24 | CR-24 | 95 | 42 | 15 | 3.3 | 90 | 12.6 | C |
| 4-25 | CR-25 | 100 | 39 | 12 | 3.3 | 90 | 12.6 | B |
| 4-26 | CR-26 | 105 | 39 | 14 | 2.7 | 80 | 13.2 | C |
| 4-27 | CR-27 | 100 | 38 | 13 | 3.0 | 90 | 12.6 | B |
| 4-28 | CR-28 | 95 | 39 | 15 | 3.2 | 90 | 12.7 | C |
| 4-29 | CR-29 | 100 | 42 | 14 | 3.2 | 90 | 12.6 | C |
| 4-30 | CR-30 | 95 | 38 | 13 | 3.1 | 90 | 12.4 | B |
| 4-31 | CR-31 | 100 | 38 | 12 | 2.7 | 90 | 12.6 | C |
| 4-32 | CR-32 | 95 | 40 | 13 | 3.1 | 80 | 13.2 | C |
| 4-33 | CR-33 | 95 | 39 | 14 | 3.4 | 90 | 12.7 | C |
| 4-34 | CR-34 | 100 | 38 | 13 | 3.3 | 80 | 13.1 | B |
| 4-35 | CR-35 | 100 | 39 | 15 | 3.4 | 90 | 12.8 | C |
| 4-36 | CR-36 | 105 | 37 | 13 | 2.7 | 70 | 13.2 | C |
| 4-37 | CR-37 | 95 | 38 | 14 | 3.7 | 70 | 13.4 | B |
| 4-38 | CR-38 | 100 | 40 | 13 | 2.9 | 70 | 13.1 | C |
| 4-39 | CR-39 | 95 | 38 | 13 | 3.5 | 70 | 13.2 | C |
| 4-40 | CR-40 | 100 | 38 | 14 | 3.4 | 70 | 14.2 | B |

From the results presented in Tables 7 to 9, it has been found that a chemically amplified resist composition using a base polymer containing a repeat unit derived from the onium salt monomer of the present invention has good sensitivity and is excellent in EL, LWR, and DOF. In addition, it has been confirmed that the chemically amplified resist composition has a small value of collapse limit and is resistant to pattern collapse even in fine pattern formation. Furthermore, it has been confirmed that development defects are also suppressed. Therefore, it has been indicated that the chemically amplified resist composition of the present invention is suitable as a material for EUV lithography.

### EUV Lithography Evaluation (2)

### Examples 5-1 to 5-53 and Comparative Examples 5-1 to 5-40

Each of the chemically amplified resist compositions (R-1 to R-53 and CR-1 to CR-10) presented in Tables 4 to 6 was spin-coated on a Si substrate formed with a silicon-containing spin-on hard mask SHB-A940 (silicon content: 43 wt %) manufactured by Shin-Etsu Chemical Co., Ltd. in a film thickness of 20 nm, and prebaked at 105° C. for 60 seconds using a hot plate to prepare a resist film having a film thickness of 50 nm. The resist film was exposed to light using an EUV scanner NXE3400 (NA: 0.33, σ: 0.9/0.6, quadrupole illumination, mask of hole pattern having an on-wafer dimension of 46 nm as a pitch and +20% bias) manufactured by ASML, PEB was performed at temperatures described in Tables 7 and 8 for 60 seconds using a hot plate, and development was performed with a 2.38 wt % TMAH aqueous solution for 30 seconds to form a hole pattern having a dimension of 23 nm.

Using a length measuring SEM (CG6300) manufactured by Hitachi High-Tech Corporation, an exposure dose when a hole was formed with a dimension of 23 nm was measured and taken as sensitivity, the dimensions of 50 holes at this time were measured, and a value (3σ) three times a standard deviation (σ) calculated from the result was taken as CDU. Results thereof are presented in Tables 10 to 12.

TABLE 10

| | | | | Optimum | |
|---|---|---|---|---|---|
| | | | PEB | exposure | |
| | | Resist | temp. | dose | CDU |
| | | composition | (° C.) | (mJ/cm²) | (nm) |
| Example | 5-1 | R-1 | 95 | 22 | 2.3 |
| | 5-2 | R-2 | 95 | 23 | 2.4 |
| | 5-3 | R-3 | 90 | 24 | 2.3 |
| | 5-4 | R-4 | 90 | 25 | 2.2 |
| | 5-5 | R-5 | 90 | 23 | 2.2 |
| | 5-6 | R-6 | 95 | 22 | 2.3 |
| | 5-7 | R-7 | 95 | 23 | 2.4 |
| | 5-8 | R-8 | 90 | 22 | 2.3 |
| | 5-9 | R-9 | 95 | 22 | 2.3 |
| | 5-10 | R-10 | 95 | 23 | 2.5 |
| | 5-11 | R-11 | 95 | 24 | 2.3 |
| | 5-12 | R-12 | 90 | 22 | 2.2 |
| | 5-13 | R-13 | 90 | 23 | 2.5 |
| | 5-14 | R-14 | 90 | 22 | 2.3 |
| | 5-15 | R-15 | 90 | 23 | 2.2 |
| | 5-16 | R-16 | 85 | 22 | 2.3 |
| | 5-17 | R-17 | 95 | 22 | 2.2 |
| | 5-18 | R-18 | 95 | 23 | 2.4 |
| | 5-19 | R-19 | 90 | 24 | 2.2 |
| | 5-20 | R-20 | 95 | 22 | 2.3 |
| | 5-21 | R-21 | 95 | 25 | 2.3 |
| | 5-22 | R-22 | 95 | 24 | 2.5 |
| | 5-23 | R-23 | 95 | 25 | 2.4 |
| | 5-24 | R-24 | 95 | 22 | 2.3 |
| | 5-25 | R-25 | 95 | 22 | 2.5 |
| | 5-26 | R-26 | 95 | 23 | 2.4 |
| | 5-27 | R-27 | 95 | 22 | 2.2 |
| | 5-28 | R-28 | 95 | 24 | 2.3 |
| | 5-29 | R-29 | 90 | 22 | 2.2 |
| | 5-30 | R-30 | 95 | 22 | 2.3 |

TABLE 11

| | | | | Optimum | |
|---|---|---|---|---|---|
| | | | PEB | exposure | |
| | | Resist | temp. | dose | CDU |
| | | composition | (° C.) | (mJ/cm²) | (nm) |
| Example | 5-31 | R-31 | 95 | 23 | 2.4 |
| | 5-32 | R-32 | 95 | 23 | 2.2 |
| | 5-33 | R-33 | 95 | 22 | 2.2 |
| | 5-34 | R-34 | 90 | 23 | 2.3 |
| | 5-35 | R-35 | 95 | 23 | 2.2 |
| | 5-36 | R-36 | 95 | 25 | 2.4 |
| | 5-37 | R-37 | 90 | 24 | 2.3 |
| | 5-38 | R-38 | 90 | 23 | 2.3 |
| | 5-39 | R-39 | 95 | 22 | 2.5 |
| | 5-40 | R-40 | 95 | 23 | 2.3 |
| | 5-41 | R-41 | 95 | 22 | 2.4 |
| | 5-42 | R-42 | 90 | 24 | 2.4 |
| | 5-43 | R-43 | 95 | 22 | 2.2 |
| | 5-44 | R-44 | 95 | 25 | 2.4 |
| | 5-45 | R-45 | 90 | 24 | 2.4 |
| | 5-46 | R-46 | 95 | 23 | 2.2 |
| | 5-47 | R-47 | 90 | 22 | 2.3 |
| | 5-48 | R-48 | 90 | 22 | 2.4 |
| | 5-49 | R-49 | 95 | 24 | 2.5 |
| | 5-50 | R-50 | 90 | 25 | 2.3 |
| | 5-51 | R-51 | 95 | 23 | 2.4 |
| | 5-52 | R-52 | 95 | 23 | 2.2 |
| | 5-53 | R-53 | 95 | 24 | 2.2 |

TABLE 12

| | | | | Optimum | |
|---|---|---|---|---|---|
| | | | PEB | exposure | |
| | | Resist | temp. | dose | CDU |
| | | composition | (° C.) | (mJ/cm²) | (nm) |
| Comparative | 5-1 | CR-1 | 95 | 31 | 2.8 |
| Example | 5-2 | CR-2 | 90 | 29 | 2.9 |
| | 5-3 | CR-3 | 90 | 28 | 3.0 |
| | 5-4 | CR-4 | 95 | 28 | 2.8 |
| | 5-5 | CR-5 | 90 | 29 | 2.9 |
| | 5-6 | CR-6 | 95 | 27 | 2.8 |
| | 5-7 | CR-7 | 95 | 27 | 2.8 |
| | 5-8 | CR-8 | 95 | 29 | 2.8 |
| | 5-9 | CR-9 | 90 | 28 | 2.8 |
| | 5-10 | CR-10 | 95 | 30 | 2.9 |
| | 5-11 | CR-11 | 95 | 29 | 2.9 |
| | 5-12 | CR-12 | 90 | 29 | 2.7 |
| | 5-13 | CR-13 | 90 | 28 | 2.8 |
| | 5-14 | CR-14 | 90 | 28 | 2.8 |
| | 5-15 | CR-15 | 95 | 27 | 3.0 |
| | 5-16 | CR-16 | 85 | 32 | 2.8 |
| | 5-17 | CR-17 | 95 | 28 | 2.9 |
| | 5-18 | CR-18 | 95 | 28 | 2.7 |
| | 5-19 | CR-19 | 90 | 29 | 2.8 |
| | 5-20 | CR-20 | 95 | 27 | 2.8 |
| | 5-21 | CR-21 | 95 | 27 | 2.8 |
| | 5-22 | CR-22 | 95 | 28 | 2.9 |
| | 5-23 | CR-23 | 95 | 28 | 2.9 |
| | 5-24 | CR-24 | 95 | 29 | 2.7 |
| | 5-25 | CR-25 | 95 | 27 | 2.8 |
| | 5-26 | CR-26 | 95 | 28 | 2.7 |
| | 5-27 | CR-27 | 95 | 27 | 2.8 |
| | 5-28 | CR-28 | 95 | 28 | 2.9 |
| | 5-29 | CR-29 | 90 | 28 | 2.9 |
| | 5-30 | CR-30 | 90 | 29 | 2.8 |
| | 5-31 | CR-31 | 95 | 27 | 2.8 |
| | 5-32 | CR-32 | 90 | 30 | 2.7 |
| | 5-33 | CR-33 | 95 | 29 | 2.8 |
| | 5-34 | CR-34 | 95 | 29 | 2.7 |
| | 5-35 | CR-35 | 95 | 27 | 2.8 |
| | 5-36 | CR-36 | 95 | 29 | 2.9 |
| | 5-37 | CR-37 | 90 | 31 | 3.1 |
| | 5-38 | CR-38 | 90 | 33 | 3.2 |
| | 5-39 | CR-39 | 90 | 33 | 3.2 |
| | 5-40 | CR-40 | 90 | 32 | 3.3 |

From the results presented in Tables 10 to 12, it has been confirmed that the chemically amplified resist composition of the present invention has good sensitivity and is excellent in CDU.

### [6] Evaluation of Dry Etching Resistance

### Examples 6-1 to 6-50 and Comparative Examples 6-1 to 6-38

A polymer solution obtained by dissolving 2 g of each of the polymers (polymers P-1 to P-50 and comparative polymers CP-1 to CP-10) presented in Tables 1 and 2 in 10 g of cyclohexanone and filtering the solution through a 0.2 μm-sized filter was deposited on a Si substrate by spin coating to form a film having a thickness of 300 nm, and evaluation was performed under the following conditions.

Etching test with CHF₃/CF₄-based gas:

A film thickness difference of the polymer film before and after etching was determined using a dry etching apparatus TE-8500P manufactured by Tokyo Electron Limited.

Etching conditions are as follows.

| | | |
|---|---|---|
| Chamber pressure: | 40 | Pa |
| RF power: | 1000 | W |
| Gap: | 9 | mm |
| CHF₃ gas flow rate: | 30 | mL/min |
| CF₄ gas flow rate: | 30 | mL/min |
| Ar gas flow rate: | 100 | mL/min |
| Time: | 60 | sec |

This evaluation indicates that a film having a small film thickness difference, that is, a film having a small reduction amount has high etching resistance.

Results of dry etching resistance are presented in Tables 13 to 15.

TABLE 13

| | | | CHF₃/CF₄-based |
|---|---|---|---|
| | | | gas etching rate |
| | | Polymer | (nm/min) |
| Example | 6-1 | P-1 | 94 |
| | 6-2 | P-2 | 92 |
| | 6-3 | P-3 | 94 |
| | 6-4 | P-4 | 96 |
| | 6-5 | P-5 | 94 |
| | 6-6 | P-6 | 95 |
| | 6-7 | P-7 | 96 |
| | 6-8 | P-8 | 93 |
| | 6-9 | P-9 | 96 |
| | 6-10 | P-10 | 97 |
| | 6-11 | P-11 | 94 |
| | 6-12 | P-12 | 96 |
| | 6-13 | P-13 | 94 |
| | 6-14 | P-14 | 96 |
| | 6-15 | P-15 | 95 |
| | 6-16 | P-16 | 96 |
| | 6-17 | P-17 | 94 |
| | 6-18 | P-18 | 93 |
| | 6-19 | P-19 | 95 |
| | 6-20 | P-20 | 95 |
| | 6-21 | P-21 | 95 |
| | 6-22 | P-22 | 96 |
| | 6-23 | P-23 | 94 |
| | 6-24 | P-24 | 95 |
| | 6-25 | P-25 | 94 |
| | 6-26 | P-26 | 94 |
| | 6-27 | P-27 | 95 |
| | 6-28 | P-28 | 96 |
| | 6-29 | P-29 | 94 |
| | 6-30 | P-30 | 95 |

TABLE 14

| | | | CHF₃/CF₄-based |
|---|---|---|---|
| | | | gas etching rate |
| | | Polymer | (nm/min) |
| Example | 6-31 | P-31 | 94 |
| | 6-32 | P-32 | 95 |
| | 6-33 | P-33 | 96 |
| | 6-34 | P-34 | 97 |
| | 6-35 | P-35 | 95 |
| | 6-36 | P-36 | 96 |
| | 6-37 | P-37 | 95 |
| | 6-38 | P-38 | 96 |
| | 6-39 | P-39 | 94 |
| | 6-40 | P-40 | 95 |
| | 6-41 | P-41 | 94 |
| | 6-42 | P-42 | 95 |
| | 6-43 | P-43 | 97 |
| | 6-44 | P-44 | 96 |
| | 6-45 | P-45 | 95 |
| | 6-46 | P-46 | 94 |
| | 6-47 | P-47 | 95 |
| | 6-48 | P-48 | 95 |
| | 6-49 | P-49 | 94 |
| | 6-50 | P-50 | 95 |

TABLE 15

| | | | CHF₃/CF₄-based |
|---|---|---|---|
| | | | gas etching rate |
| | | Polymer | (nm/min) |
| Comparative | 6-1 | CP-1 | 104 |
| Example | 6-2 | CP-2 | 102 |
| | 6-3 | CP-3 | 102 |
| | 6-4 | CP-4 | 103 |
| | 6-5 | CP-5 | 101 |
| | 6-6 | CP-6 | 101 |
| | 6-7 | CP-7 | 104 |
| | 6-8 | CP-8 | 103 |
| | 6-9 | CP-9 | 102 |
| | 6-10 | CP-10 | 101 |
| | 6-11 | CP-11 | 104 |
| | 6-12 | CP-12 | 106 |
| | 6-13 | CP-13 | 104 |
| | 6-14 | CP-14 | 108 |
| | 6-15 | CP-15 | 105 |
| | 6-16 | CP-16 | 107 |
| | 6-17 | CP-17 | 106 |
| | 6-18 | CP-18 | 106 |
| | 6-19 | CP-19 | 104 |
| | 6-20 | CP-20 | 102 |
| | 6-21 | CP-21 | 104 |
| | 6-22 | CP-22 | 102 |
| | 6-23 | CP-23 | 104 |
| | 6-24 | CP-24 | 104 |
| | 6-25 | CP-25 | 105 |
| | 6-26 | CP-26 | 103 |
| | 6-27 | CP-27 | 107 |
| | 6-28 | CP-28 | 105 |
| | 6-29 | CP-29 | 104 |
| | 6-30 | CP-30 | 102 |
| | 6-31 | CP-31 | 108 |
| | 6-32 | CP-32 | 107 |
| | 6-33 | CP-33 | 105 |
| | 6-34 | CP-34 | 104 |
| | 6-35 | CP-35 | 105 |
| | 6-36 | CP-36 | 106 |
| | 6-37 | CP-37 | 103 |
| | 6-38 | CP-38 | 114 |

From the results presented in Tables 13 to 15, it has been confirmed that the polymer of the present invention has excellent dry etching resistance to a CHF₃/CF₄-based gas.

Japanese Patent Application No. 2025-010463 is incorporated herein by reference.

Although some preferred embodiments have been described, many modifications and variations may be made thereto in light of the above teachings. It is therefore to be understood that the invention may be practiced otherwise than as specifically described without departing from the scope of the appended claims.

1. A sulfonium salt monomer having the following formula (a): 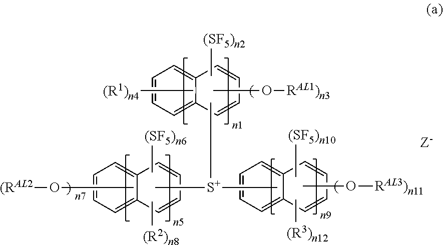
wherein n1 is 0 or 1, n2 is 0, 1, or 2, n3 is 0, 1, or 2, n4 is 0, 1, or 2, provided that 0≤n2+n3+n4≤5 when n1 is 0, and 0≤n2+n3+n4≤7 when n1 is 1, n5 is 0 or 1, n6 is 0, 1, or 2, n7 is 0, 1, or 2, n8 is 0, 1, or 2, provided that 0≤n6+n7+n8≤5 when n5 is 0, and 0≤n6+n7+n8≤7 when n5 is 1, n9 is 0 or 1, n10 is 0, 1, or 2, n11 is 0, 1, or 2, n12 is 0, 1, or 2, provided that 0≤n10+n11+n12≤5 when n9 is 0, and 0≤n10+n11+n12≤7 when n9 is 1, 1≤n2+n6+n10≤6, 1≤n3+n7+n11≤6,
R¹, R², and R³ are each independently a halogen atom, a nitro group, a cyano group, a hydroxy group, a carboxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n4 is 2, R¹s may be the same as or different from each other, and two R¹s may be bonded to each other to form a ring together with carbon atoms to which they are bonded, when n8 is 2, R²s may be the same as or different from each other, and two R²s may be bonded to each other to form a ring together with carbon atoms to which they are bonded, when n12 is 2, R³s may be the same as or different from each other, and two R³s may be bonded to each other to form a ring together with carbon atoms to which they are bonded,
R^{AL1}, R^{AL2}, and R^{AL3} are each independently an acid-unstable group, when n3 is 2, R^{AL1}s may be the same as or different from each other, when n7 is 2, R^{AL2}s may be the same as or different from each other, when n11 is 2, R^{AL3}s may be the same as or different from each other,
at least one-SFs group is bonded to a carbon atom adjacent to a carbon atom to which-O—R^{AL1}, —O—R^{AL2}, or—O—R^{AL3} is bonded,
two of the three aromatic rings bonded to S⁺ may be bonded to each other to form a ring together with the sulfur atom to which they are bonded, and
Z⁻ is a carboxylic acid anion having an aromatic vinyl structure and an iodine atom.

2. The sulfonium salt monomer according to claim 1, having the following formula (a1): 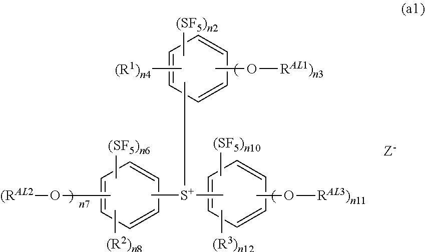
wherein n2 to n4, n6 to n8, n10 to n12, R^{AL1} to R^{AL3}, R¹ to R³, and Z are as defined above.

3. The sulfonium salt monomer according to claim 1, wherein the acid-unstable group has the following formula (AL-1) or (AL-2): 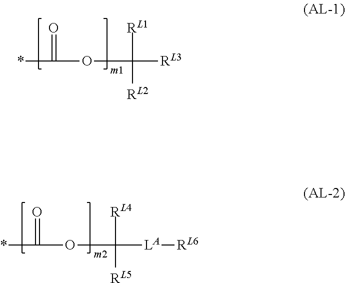
wherein m1 and m2 are each independently 0 or 1,
R^{L1} and R¹² are each independently a C₁-C₁₂ hydrocarbyl group, R^{L3} is a hydrogen atom or a C₁-C₁₂ hydrocarbyl group, some of (—CH₂—)s of the hydrocarbyl group of R^{L1}, R^{L2}, and R^{L3} may be replaced with —O— or —S—, when the hydrocarbyl group contains an aromatic ring, some or all of hydrogen atoms of the aromatic ring may be replaced with a halogen atom, a cyano group, a nitro group, a C₁-C₄ alkyl group which may contain a halogen atom, or a C₁-C₄ alkoxy group which may contain a halogen atom, R^{L1} and R^{L2} may be bonded to each other to form a ring together with a carbon atom to which they are bonded, some of (—CH₂—)s of the ring may be replaced with —O— or —S—, provided that when R^{L3} is a hydrogen atom, R^{L1} and R¹² are bonded to each other to form an alicyclic ring containing a multiple bond together with a carbon atom to which they are bonded,
R^{L4} and R^{L5} are each independently a hydrogen atom or a C₁-C₁₀ hydrocarbyl group, R^{L6} is a C₁-C₂₀ hydrocarbyl group, some of (—CH₂—)s of the hydrocarbyl group may be replaced with —O— or —S—, R^{L5} and R^{L6} may be bonded to each other to form a C₃-C₂₀ heterocyclic group together with a carbon atom to which they are bonded and L^{A}, some of (—CH₂—)s of the heterocyclic group may be replaced with —O— or —S—,
L^{A} is —O— or —S—, and
* represents a bond with adjacent-O—.

4. The sulfonium salt monomer according to claim 1, wherein Z⁻ is an anion having the following formula (Z): 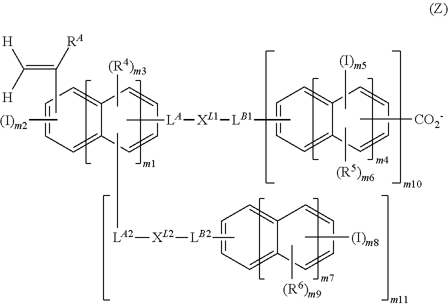
wherein m1 is 0 or 1, m2 is 0, 1, 2, 3, or 4, m3 is 0, 1, 2, or 3, m4 is 0 or 1, m5 is 0, 1, 2, 3, or 4, m6 is 0, 1, 2, or 3, m7 is 0 or 1, m8 is 1, 2, 3, or 4, m9 is 0, 1, 2, or 3, m10 is 0 or 1, m11 is 0 or 1, provided that 0≤m2+m3+m11≤4 when m1 is 0, and 0≤m2+m3+m11≤6 when m1 is 1, 0≤m5+m6≤4 when m4 is 0, and 0≤m5+m6≤6 when m4 is 1, 0≤m8+m9≤5 when m7 is 0, and 0≤m8+m9≤7 when m7 is 1, 1≤m2+m5+m8≤4,
R^{A} is a hydrogen atom, a fluorine atom, a methyl group, or a trifluoromethyl group,
R⁴, R⁵, and R⁶ are each independently a halogen atom other than an iodine atom, a nitro group, a cyano group, a hydroxy group, a carboxy group, a pentafluorosulfanyl group, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₁-C₂₀ hydrocarbyloxycarbonyl group which may contain a heteroatom, when m3 is 2 or 3, R⁴s may be the same as or different from each other, and two R⁴s may be bonded to each other to form a ring together with carbon atoms to which they are bonded, when m6 is 2 or 3, R⁵s may be the same as or different from each other, and two R⁵s may be bonded to each other to form a ring together with carbon atoms to which they are bonded, when m9 is 2 or 3, R⁶s may be the same as or different from each other, and two R⁶s may be bonded to each other to form a ring together with carbon atoms to which they are bonded,
L^{A1}, L^{A2}, L^{B1}, and L^{B2} are each independently a single bond, an ether bond, a carbonyl group, an ester bond, a sulfonate bond, an amide bond, a sulfonamide bond, a carbonate bond, or a carbamate bond, and
X^{L1} and X¹² are each independently a single bond or a C₁-C₄₀ hydrocarbylene group which may contain a heteroatom.

5. A sulfonium salt quencher comprising the sulfonium salt monomer according to claim 1.

6. A polymer comprising a repeat unit derived from the sulfonium salt quencher according to claim 5.

7. The polymer according to claim 6, further comprising at least one selected from a repeat unit having the following formula (b1), a repeat unit having the following formula (b2), and a repeat unit having the following formula (b3): 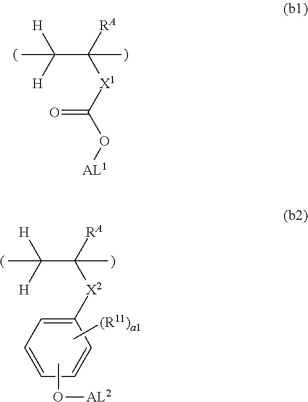
wherein R^{A}s are each independently a hydrogen atom, a fluorine atom, a methyl group, or a trifluoromethyl group,
X¹ is a single bond, a phenylene group, a naphthylene group, *—C(═O)—O—X¹¹—, or *—C(═O)—N(H)—X¹¹—, the phenylene group or the naphthylene group may be replaced with a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom, X¹¹ is a C₁-C₁₀ saturated hydrocarbylene group, a phenylene group, or a naphthylene group, the saturated hydrocarbylene group may contain a hydroxy group, an ether bond, an ester bond, or a lactone ring,
X² is a single bond, *—C(═O)—O—, or *—C(═O)—N(H)—,
* represents a bond with a carbon atom of a main chain,
R¹¹ is a halogen atom, a cyano group, a hydroxy group, a nitro group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, when a1 is 2, 3, or 4, R¹¹s may be the same as or different from each other,
AL¹ and AL² are each independently an acid-unstable group, and
a1 is 0, 1, 2, 3, or 4. 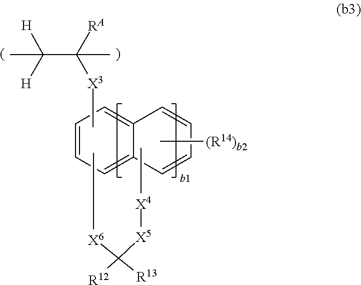
wherein b1 is 0 or 1, when b1 is 0, b2 is 0, 1, 2, or 3, and when b1 is 1, b2 is 0, 1, 2, 3, 4, or 5,
R^{A} is a hydrogen atom, a fluorine atom, a methyl group, or a trifluoromethyl group,
X³ is a single bond, *—C(═O)—O—, or *—C(═O)—N(H)—, * represents a bond with a carbon atom of a main chain,
X⁴ is a single bond, a C₁-C₄ aliphatic hydrocarbylene group, a carbonyl group, a sulfonyl group, or a group obtained by combining these groups,
X⁵ and X⁶ are each independently an oxygen atom or a sulfur atom, provided that X⁴ and X⁶ are bonded to adjacent aromatic ring carbon atoms,
R¹² and R¹³ are each independently a hydrogen atom or a C₁-C₂₀ hydrocarbyl group which may contain a heteroatom, R¹² and R¹³ may be bonded to each other to form a ring together with a carbon atom to which they are bonded,
R¹⁴ is a halogen atom, a hydroxy group, a cyano group, a nitro group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A}) (R^{14B}), R^{14A} and R^{14B} are each independently a hydrogen atom or a C₁-C₆ hydrocarbyl group, and when b2 is 2 or more, R¹⁴s may be the same as or different from each other, and a plurality of R¹⁴s may be bonded to each other to form a ring together with aromatic ring carbon atoms to which they are bonded.

8. The polymer according to claim 6, further comprising a repeat unit having the following formula (c): 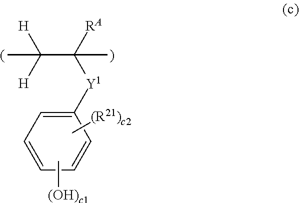
wherein R^{A} is a hydrogen atom, a fluorine atom, a methyl group, or a trifluoromethyl group,
Y¹ is a single bond, *—C(═O)—O—, or *—C(═O)—N(H)—, * represents a bond with a carbon atom of a main chain,
R²¹ is a halogen atom, a carboxy group, a nitro group, a cyano group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom,
c1 is 1, 2, 3, or 4, and c2 is 0, 1, 2, or 3, provided that 1≤c1+c2≤5.

9. The polymer according to claim 6, further comprising a repeat unit derived from an onium salt monomer containing a fluorosulfonic acid anion having a polymerizable group and at least one iodine atom and a sulfonium cation.

10. The polymer according to claim 6, further comprising a repeat unit having the following formula (e): 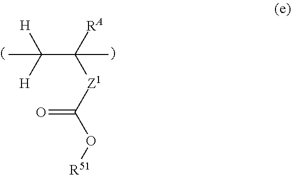
wherein R^{A} is a hydrogen atom, a fluorine atom, a methyl group, or a trifluoromethyl group,
z1 is a single bond, a phenylene group, a naphthylene group, *—C(═O)—O—Z¹¹—, or *—C(═O)—N(H)—Z¹¹—, the phenylene group or the naphthylene group may be substituted with a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom, * represents a bond with a carbon atom of a main chain, Z¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring, and
R⁵¹ is a hydrogen atom or a C₁-C₂₀ group containing at least one structure selected from a hydroxy group other than a phenolic hydroxy group, a cyano group, a carbonyl group, a carboxy group, an ether bond, an ester bond, a sulfonate bond, a carbonate bond, a lactone ring, a sultone ring, and a carboxylic anhydride (—C(═O)—O—C(═O)—).

11. A chemically amplified resist composition comprising (A) a base polymer containing the polymer according to claim 6.

12. The chemically amplified resist composition according to claim 11, further comprising (B) an organic solvent.

13. The chemically amplified resist composition according to claim 11, further comprising (C) a quencher.

14. The chemically amplified resist composition according to claim 11, further comprising (D) an acid generator.

15. The chemically amplified resist composition according to claim 11, further comprising (E) a surfactant.

16. A pattern forming method comprising: a step of forming a resist film on a substrate using the chemically amplified resist composition according to claim 11; a step of exposing the resist film to a high energy ray; and a step of developing the exposed resist film using a developer.

17. The pattern forming method according to claim 16, wherein the high energy ray is ArF excimer laser having a wavelength of 193 nm, KrF excimer laser having a wavelength of 248 nm, an electron beam, or an extreme ultraviolet ray having a wavelength of 3 to 15 nm.
